(19) United States
(12) Patent Application Publication
(10) Pub. No.: US 2004/0053385 A1
(43) Pub. Date: Mar. 18, 2004

(54) Crystal structure

(76) Inventors: Tom L. Blundell, Royston (GB); Christopher Abell, Cambridge (GB); Tsuyoshi Inoue, Cambridge (GB); Frank Von Delft, San Diego, CA (US)

Correspondence Address: NIXON & VANDERHYE P.C., 1100 North Glebe Road, 8th Floor, Arlington, VA, 22201-4714, US

(21) Appl. No.: 09/820,745

(22) Filed: Mar. 30, 2001

(51) Int. Cl. C12N 9/10; G06G 7/48; G06G 7/58; G06F 19/00

(52) U.S. Cl. 435/193; 702/19; 703/11

(57) ABSTRACT

The invention provides a crystal of ketopantoate hydroxymethyl-transferase (KPHMT) having a monoclinic space group P2₁, and unit cell dimensions of a=86.1±0.2 Å, b=157.2±0.2 Å, c=100.2±0.2 Å and β=97.4±0.2°. The crystal is further characterised by the three dimensional atomic coordinates of Table 1. The invention also provides the use of such crystals and the Table of coordinates in computer modelling applications, for example in drug discovery.

### FIELD OF THE INVENTION

The present invention relates to the enzyme ketopantoate hydroxymethyltransferase (KPHMT), and in particular its crystal structure and the use of this structure in drug discovery.

### BACKGROUND OF THE INVENTION

Pantothenic acid (vitamin B₅) is found in coenzyme A (CoA) and the acyl carrier protein (ACP), both of which are involved in fatty acid metabolism.

Pantothenic acid can be synthesised by plants and microorganisms but animals are apparently unable to make the vitamin, and require it in their diet. However, all organisms are able to convert pantothenic acid to its metabolically active form, coenzyme A.

The pathway for the synthesis of pantothenic acid is shown in FIG. 1. It provides a potential target for the treatment of infectious disease, since inhibitors of the pathway should be damaging to bacteria and fungi but not to human or animal subjects infected by such microorganisms.

Of specific interest is ketopantoate hydroxymethyl-transferase (KPHMT; 5,10-methylenetetrahydrofolate: α-ketoisovalerate hydroxymethyl transferase, EC 2.1.2.11). Powers et al. (1) showed that KPHMT is a class II aldolase that utilizes 5,10—CH₂—H₄folate (mTHF) to transfer a hydroxymethyl group to α-ketoisovalerate (α-KIVA) and thereby form ketopantoate, as shown in FIG. 2. This is the first step in pantothenic acid biosynthesis. Inhibitors (whether competitive, non-competitive, uncompetitive or irreversible) of KPHMT would be of significant technical and commercial interest.

KPHMT from Escherichia coli has been cloned and over-expressed in E. coli., and was the first sequence of a pantothenate enzyme to be determined (2). The recombinant protein has 264 amino acids, corresponding to a molecular weight of 28,237 Da. The oligomeric state of the enzyme appears to be organism specific. The homologue from the lower eukaryote, Aspergillus nidulans, has been expressed in an active form in E. coli and shown to be an octamer by gel filtration chromatography (3). However, the E. coli enzyme, was found to be a decamer by sedimentation equilibrium experiments, gel filtration chromatography and polyacrylamide gel electrophoresis under native conditions (1).

Very little is known about the mode of action of KPHMT, except that the addition of the hydroxymethyl group proceeds with retention of configuration (4). Mg²⁺ is essential for activity, whilst metal reconstitution experiments with Mn²⁺, Co²⁺ and Zn²⁺ give enzyme with progressively less activity (1). To date, five ketopantoate auxotrophs, from E. coli., A. nidulans, Daturia innoxia and two from Salmonella typhymurium, have been identified (5)(6). Four of these (from E. coli, A. nidulans, and the two from Salmonella typhymurium) have been shown to have defects in the panB gene which encodes KPHMT. The fifth (from the plant, D. innoxia) is suspected to have a panb defect (6). The A. nidulans auxotroph is caused by a deletion of Gly 168 (corresponding to Gly 205 in E. coli).

Until now no one has successfully determined the structure of KPHMT. This has prevented KPHMT inhibitors being developed via structure-based drug design methodologies. Therefore, knowledge of the structure of KPHMT would significantly assist the rational design of novel therapeutics based on KPHMT inhibitors.

Definitions

In the following by “binding site” we mean a site (such as an atom, a functional group of an amino acid residue or a plurality of such atoms and/or groups) in a KPHMT binding cavity which may bind to an agent compound such as a candidate inhibitor. Depending on the particular molecule in the cavity, sites may exhibit attractive or repulsive binding interactions, brought about by charge, steric considerations and the like.

By “fitting”, is meant determining by automatic, or semi-automatic means, interactions between one or more atoms of an agent molecule and one or more atoms or binding sites of the KPHMT, and calculating the extent to which such interactions are stable. Various computer-based methods for fitting are described further herein.

By “root mean square deviation” we mean the square root of the arithmetic mean of the squares of the deviations from the mean.

By a “computer system” we mean the hardware means, software means and data storage means used to analyse atomic coordinate data. The minimum hardware means of the computer-based systems of the present invention comprises a central processing unit (CPU), input means, output means and data storage means. Desirably a monitor is provided to visualise structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” we mean any media which can be read and accessed directly by a computer e.g. so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

### SUMMARY OF THE INVENTION

The present invention is at least partly based on overcoming several technical hurdles: we have (i) produced KPHMT crystals of suitable quality, including crystals of selenium atom KPHMT derivatives, for performing X-ray diffraction analyses, (ii) collected X-ray diffraction data from the crystals, (iii) determined the three-dimensional structure of KPHMT, and (iv) identified binding sites on the enzyme which are likely to be involved in the enzymatic reaction.

In general aspects, the present invention is concerned with identifying or obtaining agent compounds (especially inhibitors of KPHMT) for modulating KPHMT activity, and in preferred embodiments identifying or obtaining actual agent compounds/inhibitors. Crystal structure information presented herein is useful in designing potential inhibitors and modelling them or their potential interaction with the KPHMT binding cavity. Potential inhibitors may be brought into contact with KPHMT to test for ability to interact with the KPHMT binding cavity. Actual inhibitors may be identified from among potential inhibitors synthesized following design and model work performed in silico. An inhibitor identified using the present invention may be formulated into a composition, for instance a composition comprising a pharmaceutically acceptable excipient, and may be used in the manufacture of a medicament for use in a method of treatment. These and other aspects and embodiments of the present invention are discussed below.

In a first aspect, the present invention provides a crystal of KPHMT having a monoclinic space group P2₁, and unit cell dimensions of a=86.1 Å, b=157.2 Å, c=100.2 Å and β=97.4°, or more generally a=86.1±0.2 Å, b=157.2±0.2 Å, c=100.2±0.2 Å and β=97.4±0.2°.

We have found that the asymmetric unit of such a crystal corresponds to a KPHMT decamer which may be thought of as a pentamer of KPHMT dimers, the dimers being related by a non-crystallographic five-fold axis

Alternatively, or additionally, the crystal may have the three dimensional atomic coordinates of Table 1. An advantageous feature of the structural data according to Table 1 are that they have a high resolution of about 1.8 Å.

The coordinates of Table 1 provide a measure of atomic location in Angstroms, to a first decimal place. The coordinates are a relative set of positions that define a shape in three dimensions, so it is possible that an entirely different set of coordinates having a different origin and/or axes could define a similar or identical shape. Furthermore, varying the relative atomic positions of the atoms of the structure so that the root mean square deviation of the residue backbone atoms (i.e. the nitrogen-carbon-carbon backbone atoms of the protein amino acid residues) is less than 1.5 Å (preferably less than 1.0 Å and more preferably less than 0.5 Å) when superimposed on the coordinates provided in Table 1 for the residue backbone atoms, will generally result in a structure which is substantially the same as the structure of Table 1 in terms of both its structural characteristics and potency for structure-based design of KPHMT inhibitors. Likewise changing the number and/or positions of the water molecules and/or substrate molecules of Table 1 will not generally affect the potency of the structure for structure-based design of KPHMT inhibitors. Thus for the purposes described herein as being aspects of the present invention, it is within the scope of the invention if: the Table 1 coordinates are transposed to a different origin and/or axes; the relative atomic positions of the atoms of the structure are varied so that the root mean square deviation of residue backbone atoms is less than 1.5 Å (preferably less than 1.0 Å and more preferably less than 0.5 Å) when superimposed on the coordinates provided in Table 1 for the residue backbone atoms; and/or the number and/or positions of water molecules and/or substrate molecules is varied. Reference herein to the coordinate data of Table 1 thus includes the coordinate data in which one or more individual values of the Table are varied in this way.

Also, modifications in the KPHMT crystal structure due to e.g. mutations, additions, substitutions, and/or deletions of amino acid residues (including the deletion of one or more KPHMT protomers) could account for variations in the KPHMT atomic coordinates. However, atomic coordinate data of KPHMT modified so that a ligand that bound to one or more binding sites of KPHMT would be expected to bind to the corresponding binding sites of the modified KPHMT are, for the purposes described herein as being aspects of the present invention, also within the scope of the invention. Reference herein to the coordinates of Table 1 thus includes the coordinates modified in this way. Preferably, the modified coordinate data define at least one KPHMT binding cavity.

In a further aspect, the invention provides a method for crystallizing a selenomethionine KPHMT derivative which comprises producing KPHMT by recombinant production in a bacterial host (e.g. E. coli) in the presence of seleno-methionine, recovering a selenomethionine KPHMT derivative from the host and growing crystals from the recovered seleno-methionine KPHMT derivative.

Thus, the selenium atom KPHMT derivative and KPHMT produced by crystallising native KPHMT (see the detailed description below) are provided as crystallised proteins suitable for X-ray diffraction analysis.

The crystals may be grown by any suitable method, e.g. the hanging drop method.

In another aspect, the invention provides a method of analysing a KPHMT-ligand complex comprising the step of employing (i) X-ray crystallographic diffraction data from the KPHMT-ligand complex and (ii) a three-dimensional structure of KPHMT to generate a difference Fourier electron density map of the complex, the three-dimensional structure being defined by atomic coordinate data according to Table 1.

Therefore, KPHMT-ligand complexes can be crystallised and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallised KPHMT and the solved structure of un-complexed KPHMT. These maps can then be used to determine whether and where a particular ligand binds to KPHMT and/or changes the conformation of KPHMT.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualisation and model building programs such as 0 (Jones et al., Acta Crystallograhy, A47, (1991), 110-119) can be used.

In another aspect, the present invention provides a method for identifying an agent compound (e.g. an inhibitor) which modulates KPHMT activity, comprising the steps of:

(a) employing three-dimensional atomic coordinate data according to Table 1 to characterise at least a plurality of KPHMT binding sites;

(b) providing the structure of a candidate agent compound;

(c) fitting the candidate agent compound to the binding sites; and

(d) selecting the candidate agent compound.

Preferably sufficient binding sites are characterised to define a KPHMT binding cavity.

A plurality (for example two, three or four) of spaced KPHMT binding sites may be characterised and a plurality of respective compounds designed or selected. The agent compound may then be formed by linking the respective compounds into a larger compound which maintains the relative positions and orientations of the respective compounds at the binding sites. The larger compound may be formed as a real molecule or by computer modelling.

In any event, the determination of the three-dimensional structure of KPHMT provides a basis for the identification of new and specific ligands for KPHMT e.g. by computer modelling.

More specifically, a potential modulator of KPHMT activity can be examined through the use of computer modelling using a docking program such as GRAM, DOCK, or AUTODOCK (see Walters et al., Drug Discovery Today, Vol.3, No.4, (1998), 160-178, and Dunbrack et al., Folding and Design, 2, (1997), 27-42). This procedure can include computer fitting of candidate inhibitors to KPHMT to ascertain how well the shape and the chemical structure of the candidate inhibitor will bind to the enzyme.

Also computer-assisted, manual examination of the binding cavity structure of KPHMT may be performed. The use of programs such as GRID (Goodford, J. Med. Chem., 28, (1985), 849-857)—a program that determines probable interaction sites between molecules with various functional groups and the enzyme surface—may also be used to analyse the binding cavity to predict partial structures of inhibiting compounds.

Computer programs can be employed to estimate the attraction, repulsion, and steric hindrance of the two binding partners (e.g. the KPHMT and a candidate inhibitor). Generally the tighter the fit, the fewer the steric hindrances, and the greater the attractive forces, the more potent the potential modulator since these properties are consistent with a tighter binding constant. Furthermore, the more specificity in the design of a potential drug, the more likely it is that the drug will not interact with other proteins as well. This will tend to minimise potential side-effects due to unwanted interactions with other proteins.

In one embodiment a plurality of candidate agent compounds are screened or interrogated for interaction with the binding sites. In one example, step (b) involves providing the structures of the candidate agent compounds, each of which is then fitted in step (c) to computationally screen a database of compounds (such as the Cambridge Structural Database) for interaction with the binding sites. In another example, a 3-D descriptor for the agent compound is derived, the descriptor including e.g. geometric and functional constraints derived from the architecture and chemical nature of the binding cavity. The descriptor may then be used to interrogate the compound database, the identified agent compound being the compound which matches with the features of the descriptor. In effect, the descriptor is a type of virtual pharmacophore.

Having designed or selected possible binding partners, these can then be screened for activity. Consequently, the method preferably comprises the further steps of:

(e) obtaining or synthesising the candidate agent compound; and

(f) contacting the candidate agent compound with KPHMT to determine the ability of the candidate agent compound to interact with KPHMT.

In step (e) the candidate agent compound may be contacted with KPHMT in the presence of a substrate, and typically a buffer, to determine the ability of the candidate agent compound to inhibit KPHMT. The substrate may be e.g., one or both of 5,10—CH₂—H₄folate, α-ketoisovalerate, or salts thereof. So, for example, an assay mixture for KPHMT may be produced which comprises the candidate inhibitor, substrate and buffer.

Instead of, or in addition to, performing e.g. a chemical assay, the method may comprise the further steps of:

(e) obtaining or synthesising the candidate agent compound;

(f) forming a complex of KPHMT and the candidate agent compound; and

(g) analysing (e.g. by the method of an earlier aspect of the invention) said complex by X-ray crystallography or NMR spectroscopy to determine the ability of the candidate agent compound to interact with KPHMT.

Detailed structural information can then be obtained about the binding of the agent compound to KPHMT, and in the light of this information adjustments can be made to the structure or functionality of the compound, e.g. to improve binding to the binding cavity. Steps (e) to (g) may be repeated and re-repeated as necessary. For X-ray crystallographic analysis, the complex may be formed by crystal soaking or co-crystallisation.

In another aspect, the invention includes a compound which is identified as a modulator of KPHMT activity by the method of the fourth aspect.

Following identification of an inhibitor compound, it may be manufactured and/or used in the preparation, i.e. manufacture or formulation, of a composition such as a medicament, pharmaceutical composition or drug. These may be administered to individuals.

Thus, the present invention extends in various aspects not only to an inhibitor as provided by the invention, but also a pharmaceutical composition, medicament, drug or other composition comprising such an inhibitor e.g. for treatment (which may include preventative treatment) of disease such as microbial infection; a method comprising administration of such a composition to a patient, e.g. for treatment of disease such as microbial infection; use of such an inhibitor in the manufacture of a composition for administration, e.g. for treatment of disease such as microbial infection; and a method of making a pharmaceutical composition comprising admixing such an inhibitor with a pharmaceutically acceptable excipient, vehicle or carrier, and optionally other ingredients.

In another aspect, the invention relates to a method of determining three dimensional structures of KPHMT homologues of unknown structure by utilising the structural coordinates of Table 1.

For example, if X-ray crystallographic or NMR spectroscopic data is provided for a KPHMT homologue of unknown structure, the structure of KPHMT as defined by Table 1 may be used to interpret that data to provide a likely structure for the KPHMT homologue by techniques which are well known in the art, e.g. phase modelling in the case of X-ray crystallography.

One embodiment of the method comprises the steps of:

(a) aligning a representation of an amino acid sequence of a KPHMT homologue of unknown structure with the amino acid sequence of KPHMT to match homologous regions of the amino acid sequences;

(b) modelling the structure of the matched homologous regions of the KPHMT of unknown structure on the structure as defined by Table 1 of the corresponding regions of KPHMT; and

(c) determining a conformation (e.g. so that favourable interactions are formed within the KPHMT of unknown structure and/or so that a low energy conformation is formed) for the KPHMT of unknown structure which substantially preserves the structure of said matched homologous regions.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

Preferably one or all of steps (a) to (c) are performed by computer modelling.

Homology modelling is a technique that is well known to those skilled in the art (see e.g. Greer, Science, Vol. 228, (1985), 1055, and Blundell et al., Eur. J. Biochem, Vol. 172, (1988), 513).

In general, comparison of amino acid sequences is accomplished by aligning the amino acid sequence of a polypeptide of a known structure with the amino acid sequence of the polypeptide of unknown structure. Amino acids in the sequences are then compared and groups of amino acids that are homologous are grouped together. This method detects conserved regions of the polypeptides and accounts for amino acid insertions or deletions.

Homology between amino acid sequences can be determined using commercially available algorithms. The programs BLAST, gapped BLAST, BLASTN and PSI-BLAST (provided by the National Center for Biotechnology Information) are widely used in the art for this purpose, and can align homologous regions of two amino acid sequences.

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide lgeometries or by molecular simulation techniques, such as molecular dynamics (7). The final step in the process is accomplished by refining the entire structure using molecular dynamics and/or energy minimization.

In another aspect, the present invention provides systems, particularly a computer systems, intended to generate structures and/or perform rational drug design for KPHMT, KPHMT-ligand complexes or KPHMT homologues, the systems containing either (a) atomic coordinate data according to Table 1, said data defining the three-dimensional structure of KPHMT, or (b) structure factor data for KPHMT, said structure factor data being derivable from the atomic coordinate data of Table 1.

In another aspect, the present invention provides computer readable media with either (a) atomic coordinate data according to Table 1 recorded thereon, said data defining the three-dimensional structure of KPHMT, or (b) structure factor data for KPHMT recorded thereon, the structure factor data being derivable from the atomic coordinate data of Table 1.

By providing such computer readable media, the atomic coordinate data can be routinely accessed to model KPHMT. For example, RASMOL (Sayle et al., TIBS, Vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976) ), are particularly useful for calculating e.g. difference Fourier electron density maps.

### BRIEF DESCRIPTION OF THE DRAWINGS

FIG. 1 shows the pathway for the synthesis of pantothenic acid;

FIG. 2 shows the chemical reaction between α-KIVA and 5,10—CH₂—H₄folate which is catalysed by KPHMT;

FIGS. 3a and b show ribbon representations of the decameric structure as viewed respectively along and from the side of the non-crystallographic five-fold axis;

FIGS. 4a and b show ribbon representations of respectively top and side views of a protomer;

FIG. 5 shows a sequence alignment between primary structure among five members of the KPHMT family and the secondary structure of the E. coli enzyme;

FIG. 6 shows a stereo pair wire-frame electron density map of the substrate binding site with a ketopantoate product molecule (KPL) and a metal ion believed to be Mg²⁺ on which the enzyme is dependent for its activity;

FIG. 7 shows an electrostatic potential map for a protomer viewed looking towards the opening mouth of the binding cavity;

FIG. 8 shows a stereo pair ribbon representation of the KPHMT binding cavity;

FIG. 9 shows a schematic representation of the distorted octahedral binding site for Mg²⁺ in the KPHMT binding cavity;

FIGS. 10 and b show respectively side and top view stereo pair ribbon representations of the mouth of the KPHMT binding cavity; and

FIGS. 11a and b show stereo pair ribbon representations of respectively (a) the binding cavities of a KPHMT dimer, and (b) the interface between adjacent KPHMT dimers.

### DETAILED DESCRIPTION OF THE INVENTION

The present invention is founded on the determination of the three dimensional atomic structure of KPHMT.

Solving the Crystal Structure

1. Preparation of Recombinant KPHMT Protein

Cell Growth

3×15 mL starting culture of E-coli Hfr3000-YA139 cells with the plasmid pCEJ01 containing the clone pAL01 was incubated at 37° C. overnight in LB broth containing ampicillin (50 mg/mL). This was added to 3 litres of LB broth containing ampicillin (50 mg/mL) and IPTG (90 mg/mL) and incubated at 37° C. for 16 h. Selenomethionine (SeMet) protein was over-expressed in media containing selenomethionine, as well as six other amino acids (lysine, phenylalanine, threonine, isoleucine, leucine and valine) whose presence inhibit methionine biosynthesis (8) and was purified in the same way as the wild type. The cells were harvested by centrifugation at 10,000 rpm at 4° C. for 30 min. The wet cell pellet weighed approximately 9 g.

Protein Extraction

The cell pellet was resuspended in 50 mL of 100 mM phosphate buffer (pH 7) containing 1 mM DTT, 1 mM ethylenediaminetetraacetic acid (EDTA) and 1 mM phenylmethylsulphonylfluoride (PMSF) The suspension was sonicated on ice for 1 s bursts every 3 s for 12 min and the lysate centrifuged at 12,000 rpm for 30 min. Nucleic acids were removed from the supernatant by precipitation with 2% protamine sulphate (1 mL/g of cell pellet) and centrifugation at 12,000 rpm for 30 min.

The protein was precipitated from the supernatant with ammonium sulphate (25-60% saturation) and centrifugation at 12,000 rpm for 30 min. The protein pellet was dissolved in 30 mL of 100 mM phosphate buffer (pH 7) containing 1 mM DTT and 1 mM EDTA and dialysed, overnight against 25 mM phosphate buffer (pH 7) containing 1 mM DTT and 1 mM EDTA. The volume of the dialysed protein was reduced to below 20 mL by ultrafiltration.

Pellets that contained cell debris, 2% protamine sulphate precipitant and 0-25% ammonium sulphate precipitant were dissolved in a total volume of 30 mL of 100 mM phosphate buffer (pH 7) containing 1 mM DTT and 1 mM EDTA, pooled and dialysed, overnight, against 25 mM phosphate buffer (pH 7) containing 1 mM DTT and 1 mM EDTA. The volume of the dialysed mixture was reduced to below 20 mL by ultrafiltration and filtered through a 0.2 μm filter. The protein was purified by FPLC.

Hiprep Q XL Anion Exchange Chromatography

The sample, in less than 10 mL, was loaded, using a 10 mL superloop, onto a Hiprep 16/10 Q XL column equilibrated in starting buffer which consisted of 90% buffer A, containing 50 mM potassium phosphate buffer (pH 6.9), 1 mM DDT and 1 mM EDTA, and 10% buffer B containing 50 mM potassium phosphate buffer (pH 6.9), 1 mM DDT and 1 mM EDTA and 1 M potassium chloride (KCl). KPHMT was eluted in a step gradient of 0.1-1 M KCl in 12 column volumes (240 mL) and at a flow rate of 2.5 mL/min. The gradient was shaped as indicated below. KPHMT eluted in a single peak at about 0.4 M KCl. Eluate fractions were assessed for KPHMT content by SDS-PAGE. Fractions containing KPHMT were pooled and dialysed overnight against starting buffer.

Source 15Q Anion Exchange Chromatography

The sample, in less than 10 mL, was loaded, using a 10 mL superloop, onto a Source 15Q XV 16/10 column equilibrated in starting buffer which consisted of 90% buffer A, containing 25 mM potassium phosphate buffer (pH 6.9), 1 mM DDT and 1 mM EDTA, and 10% buffer B containing 25 mM potassium phosphate buffer (pH 6.9), 1 mM DDT and 1 mM EDTA and 1 M KCl. KPHMT was eluted of the Source 15Q XV 16/10 column in the same way it was eluted of the Hiprep 16/10 Q XL column. KPHMT eluted in a single peak at about 0.4 M potassium chloride.

Eluate fractions were assessed for KPHMT content by SDS-PAGE. Fractions containing KPHMT were pooled and dialysed overnight against starting buffer.

KCl gradient used in anion exchange chromatography of KPHMT:

step 1—0.1 to 0.4 M KCl (0-50 mL)

step 2—at 0.4 M KCl (50-110 mL)

step 3—0.4 to 0.5 M KCl (110-120 mL)

step 4—at 0.5 M KCl (120-180 mL)

step 5—0.5 to 1 M KCl (180-190 mL)

step 6—at 1 M KCl (190-240 mL)

Hiload 16/60 Superdex 200 pg Gel Filtration Chromatography

Sample was loaded in less than 10 mL onto a Hiload 16/60 superdex 200 pg equilibrated in buffer containing 50 mM potassium phosphate buffer (pH 6.9), 1 mM DDT and 1 mM EDTA. A constant flow rate of 0.5 mL per minute was maintained and 3 mL fractions were collected. Fractions containing KPHMT were determined by SDS-PAGE, pooled and concentrated by ultrafiltration to greater than 5 mg/mL. 26 mg of protein was obtained from a 3 L cell culture.

2. Protein Crystallisation

The sample of KPHMT was concentrated to 24 mg/ml in 40 mM of ketopantolactone (KPL; product) and 50 mM HEPES pH 7.4. Diffraction-quality single crystals of KPHMT were obtained by the hanging-vapor diffusion method at 4° C. To make a drop, one volume (1.5 μl) of protein solution was placed on a siliconised cover slide, and the equivalent reservoir solution was added at 19° C. Reservoir solution contained 9% (w/v) PEG 8000, 50 mM NaCitrate (pH 6.8), 50-100 mM Na(CH₃CO₂) and 200 mM NaCl. The plate was sealed within 1 minute and left at 4° C. After 2 hours the plate was placed into a polystyrene box, then the box was sealed and placed at 19° C. Single crystals with dimensions of about 0.5×0.3×0.1 mm appeared within one or two days. These belonged to the monoclinic space group P2₁ with cell parameters a=86.1 Å, b=157.2 Å, c=100.2 Å and β=97.4°, and accommodated one decameric enzyme per asymmetric unit, with a solvent content of 49%.

The SeMet KPHMT crystals, which were prepared in a similar way to native KPHMT crystals, seldom grew larger than 0.3 mm or thicker than about 30 μm. The SeMet KPHMT stock solution contained 2 mM KPL and 10 mM DTT to protect the Se atoms from oxidation.

3. Data Collection

The structure of KPHMT was solved by the MAD method (9) using the SeMet derivative. Data to 3.1 Å resolution were collected at 100 K, at three wavelengths on Station 19-ID of the Structural Biology Centre at the Advanced Photon Source of Argonne National Laboratory, Chicago, US. Crystals of KPHMT were cryo-protected by a protocol of gradual soaking in the cryo-protectant PEG400. Each crystal was placed in 20 ml of crystallisation solution, and the concentration of PEG400 was gradually increased to 20% (v/v) in 5% increments. The soaking time at each PEG400 concentration was a minimum of 15 minutes. At each concentration step, KPL was added to a concentration of 2 mM. The flash-cooled crystals were used for data collection.

An X-ray fluorescence spectrum was recorded and used to select wavelengths for subsequent MAD data collection. Data were collected at the Se absorption edge λe=0.97939 Å, the absorption peak λp=0.97927 Å and at remote reference wavelength λr=0.9393 Å. The diffraction data were indexed and integrated using the D*TREK suite (10), and reflexions were indexed and integrated using MOSFLM (11). The three data sets were scaled to the remote data-set using SCALA (12) and structure-factor amplitudes were calculated using TRUNCATE (13). Statistics of the processed data are listed in Table 2.

The native data set was collected to 1.8 Å resolution on Station 19-ID. A cryo-protectant solution for the native crystals contained 9% PEG8000, 50 mM NaCitrate (pH 6.8), 50-100 mM Na(CH₃CO₂), 200 mM NaCl, and 20% of PEG400.

4. Structure Determination and Refinement

160 out of the 180 Se sites in the asymmetric unit were found with the program SnB (14) using direct methods and anomalous difference data of λp SeMet. Data were phased with SHARP (15) using all three wavelength data sets, which also revealed two additional Se sites in the residual maps.

Data collected at the remote wavelength were treated as the reference data set and resolution limits of 40 to 2.3 Å were imposed. Experimental values of the anomalous dispersion (f′ and f″ in Table 2) estimated from fluorescence spectra were used and refined during analysis. The resulting values are very similar to the theoretical values and are given in Table 2. Experimental phases were improved by solvent flattening using SOLOMON (CCP4, 1994), via the SUSHI graphical user interface (La Fortelle et al., 1997) with a solvent content of 430%. The final electron-density map was easily interpretable and the whole polypeptide chain was assigned based on the initial electron density map.

The polypeptide chain was fitted in the MAD electron density map using program O (16). Rounds of maximum likelihood refinement with REFMAC (17) were alternated with visual inspection of electron density and manual rebuilding of side chains. Several rounds of simulated annealing with CNS (18) were included to refine the position of the main chain properly.

Table 1 provides the atomic coordinates of the final model.

The quality of the final model was assessed from Ramachandran plots and the analysis of the model geometry was carried out with the program PROCHECK (19). 10% of the reflections were set aside for R_{free} calculations. The plot indicated that 90.2% of the residues lay in the favourable regions and 9.8% in the allowed regions. The final R and R_{free} factors of the structure for all reflections between 75.0 and 1.8 Å resolutions were 0.229 and 0.263, respectively. The structural model for KPHMT consists of a decamer in the asymmetric unit with 2,640 amino residues, 19,830 protein atoms (non-hydrogen), 100 substrate atoms (non-hydrogen), 1,612 water molecules and 10 metal ions. The last cycle of the refinement without NCS-restrains gave a reasonable stereo-chemistry by using 229,076 unique reflections in the range of 75.0 to 1.8 Å resolution. The root mean-square deviation from standard values are 0.006 Å in bond distances (1-2 distance), 1.2° in angle distances (1-3 distance), and 22.1° in dihedral angles (planar 1-4 distance). From a Ramachandran plot the model was considered to exhibit a good stereo-chemistry.

Structural Characterisation

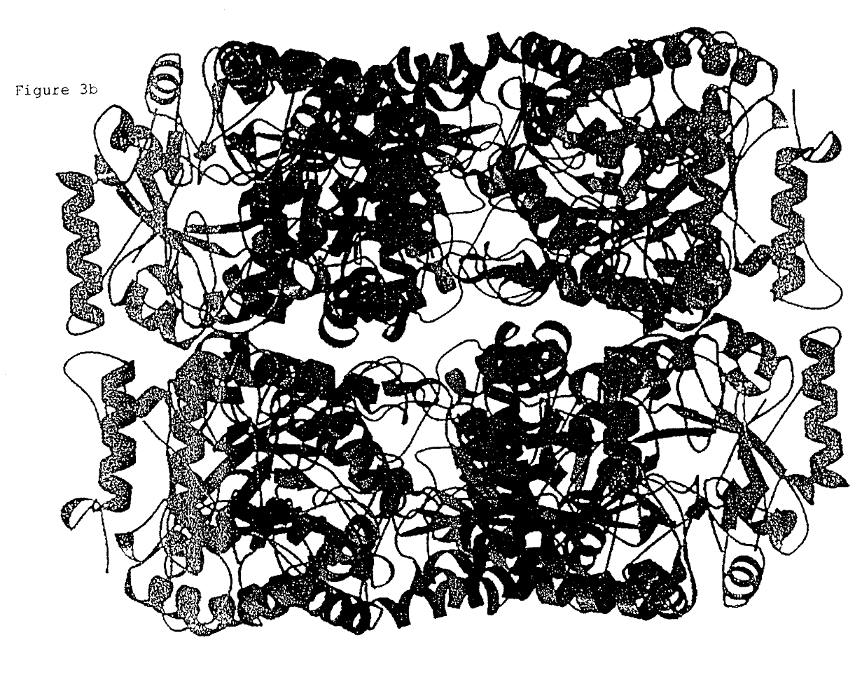

The crystal structure of KPHMT is based on a decameric asymmetric unit formed by a pentamer of dimers related by a non-crystallographic five-fold axis. FIGS. 3a and b show ribbon representations of the decameric structure as viewed respectively along and from the side of the non-crystallographic five-fold axis.

The dimensions of the decamer are approximately 100×100×75 Å. The accessible area of the decamer, 83,200 Å², is small considering the surface area for each protomer (i.e. monomer subunit), 10,800 Å², while the buried surface of each protomer is 23%. The close packing of the protomers explains the protein's remarkable resistance to denaturation by heat and urea (20). The interface between protomers in each dimeric unit is large (1140 Å²) and tightly organised, involving 88 (20 hydrophilic and 68 hydrophobic) interactions. However, the interface between protomers in the pentamer is smaller (760 Å²) and involves only 20 (6 hydrophilic and 14 hydrophobic) interactions. For this reason, we believe that the dimer is the functional unit. This is corroborated by the homologue from Aspergillus nidulans, which is an octamer (3).

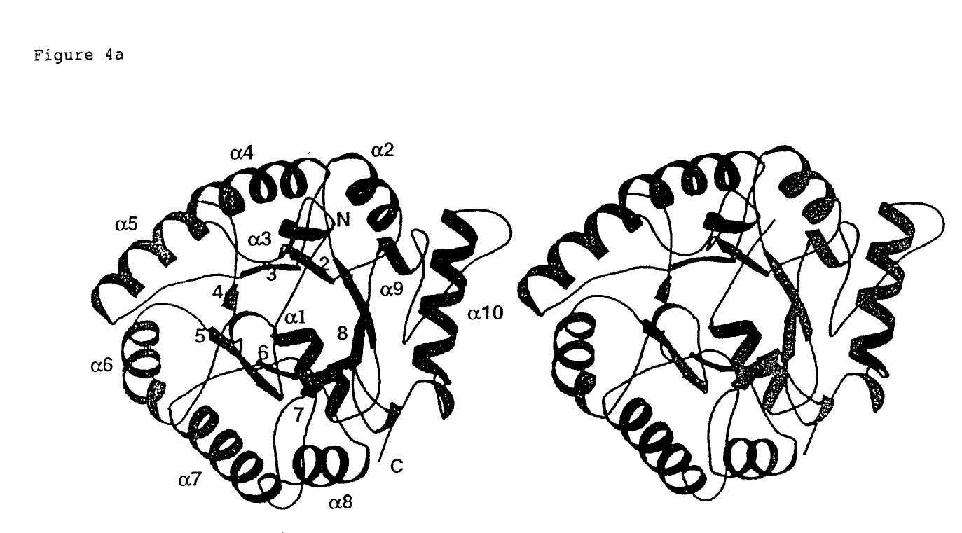

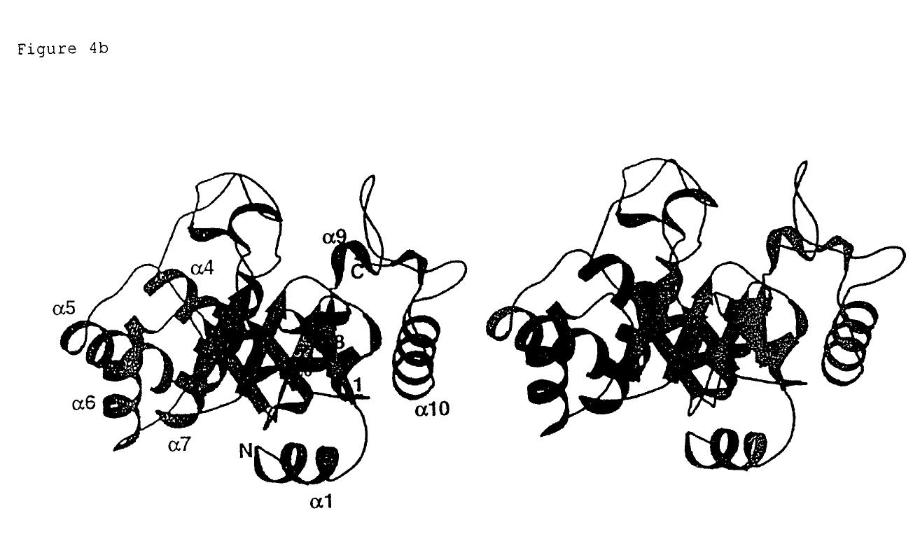

Each protomer is approximately spherical and has overall dimensions of 50×50×40 Å. Ribbon representation top and side views of a protomer are presented in FIGS. 4c and d. The tertiary structure is an α₈β₈ (TIM (triose phosphate isomerase) barrel with an extra α-helix located at the base of the β-barrel (21). The barrel consists of eight parallel β-strands surrounded by eight α-helices.

Sequence Alignment

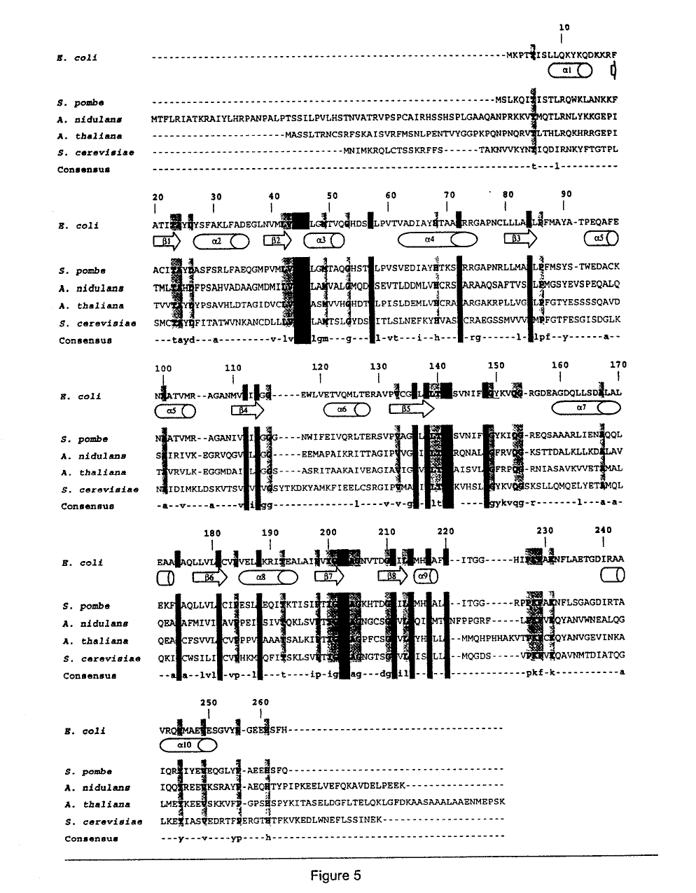

35 proteins or translated gene-sequences have been identified using a PSI-BLAST search, with high enough similarity to be classified as members of the KPHMT family (22). The enzyme is found in bacteria, lower eukaryotes (e.g. yeast) and in the plant Arabidopsis thaliana but is not found in Caenorhabditis elegans, Drosophila melanogaster or, as yet, in other higher eukaryotes. This is consistent with the end product of this pathway being a vitamin. We have analyzed the sequences from the 35 members of this family to identify residues important to the mode of action. Correlation between primary structure among five members of the KPHMT family and the secondary structure of the E. coli enzyme is shown in FIG. 5. The consensus sequence, generated by ClustalW (23) with the sequences of the 35 members, highlights that of the 264 residues, 23 residues are invariant while an additional 77 are conserved. Six conserved sequence motifs, at least six residues in length, were also identified. These are ⁴²LeuValGlyAspSerLeuGlyMet⁴⁹, ¹¹¹ValLysIleGluGlyGly¹¹⁶ ¹³⁵GlyHisXGlyLeuThrProGln¹⁴² (where X is a hydrophobic residue), ¹⁴⁸GlyGlyTyrLysValGlnGly¹⁵⁴, ²⁰⁰IleGlyIleGlyAlaGly²⁰⁵ and ²⁰⁹AspGlyAsnIleLeuVal²¹⁴. The first two of the six motifs contain residues shown in the crystal structure to be involved in binding the ketopantoate (and hence the substrate) or metal ion.

Deletion of residue Gly 168 (which corresponds to Gly 205 in the fifth motif given above) in A. nidulans has been shown to prevent cell growth (3). This residue is invariant in 34 out of the 35 KPHMT sequences and mutated to serine in a potentially inactive isoform from Pseudomonas aeruginosa. Thus, the motif may be required for correct folding of the protein.

Substrate Binding Site

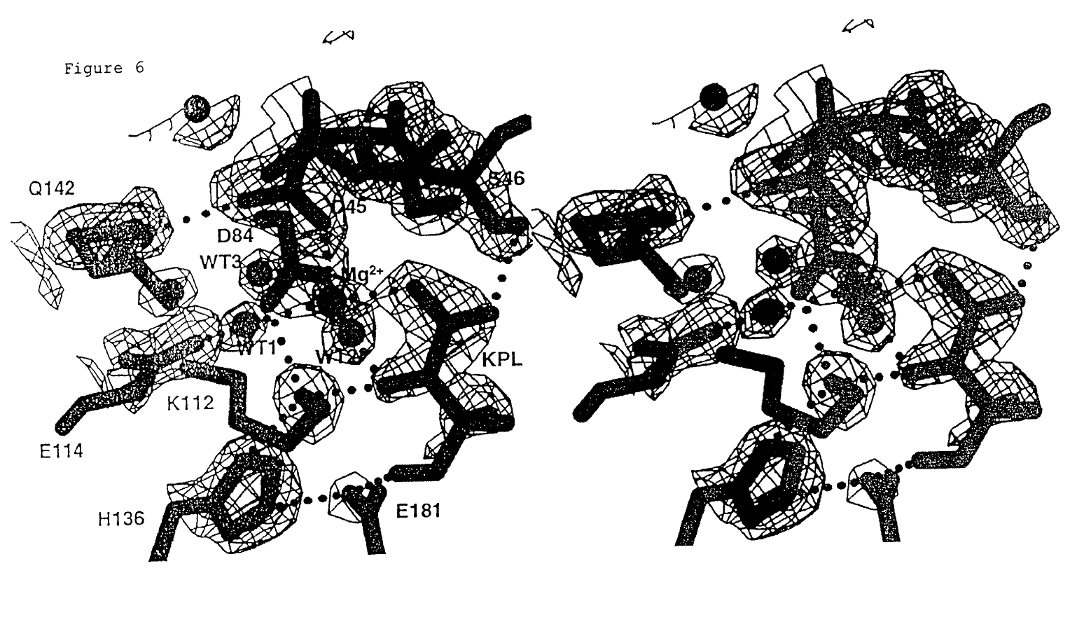

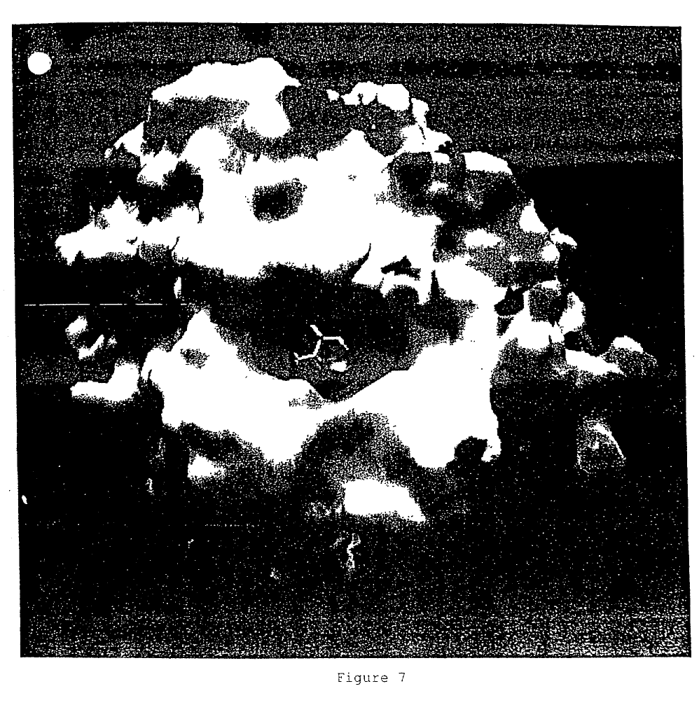

The substrate binding site is located in a large cavity at the protein C-terminus ends of the β-strands. The cavity extends almost one quarter the distance in to the protein and is about 20 Å in length and about 10 Å×15 Å in transverse section. The substrate is believed to bind before the cofactor, because the cofactor binds at the mouth of the cavity effectively blocking access to the cavity. FIG. 6 is a stereo pair wire-frame electron density map of the substrate binding site showing a ketopantoate product molecule (KPL) and a metal ion believed to be Mg²⁺ on which the enzyme is dependent for its activity.

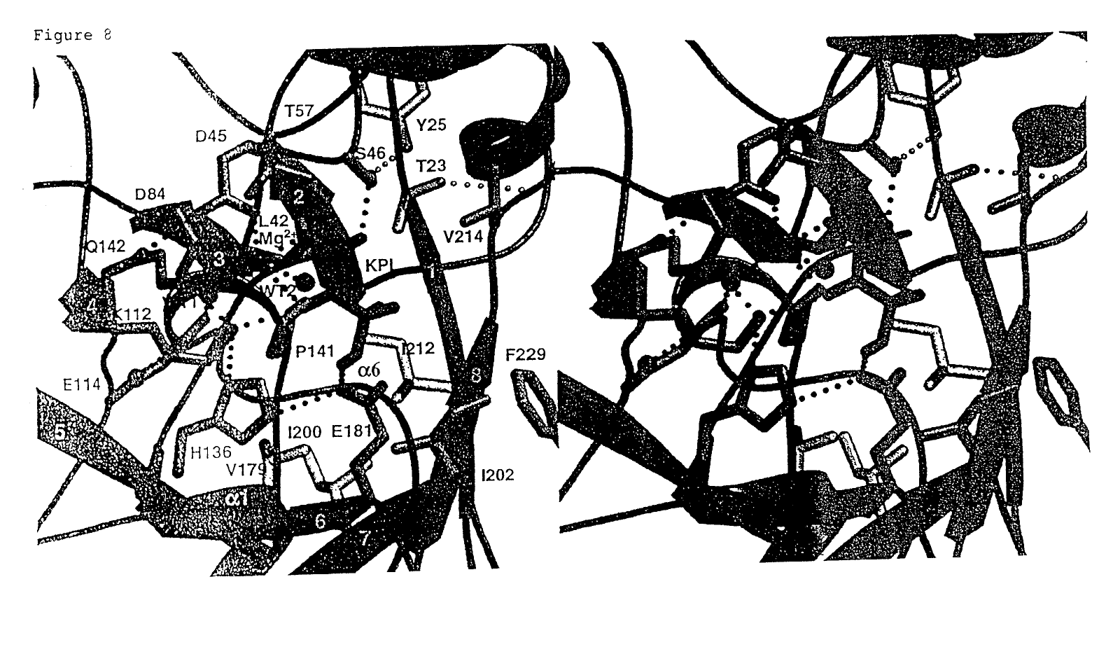

The electrostatic potential map for a protomer (shown in FIG. 7) demonstrates that the opening mouth of the binding cavity is highly charged. The surface contains eight highly conserved residues that hydrogen bond to each other and the substrate or product. As shown in FIG. 8, which is a stereo pair ribbon representation of the binding cavity, Asp 45 and Asp 84 hydrogen bond to Gln 142 and Lys 112, respectively, while Ser 46, Glu 181 and Lys 112 hydrogen bond to ketopantoate and the residues Tyr 25, His 136 and Asp 84.

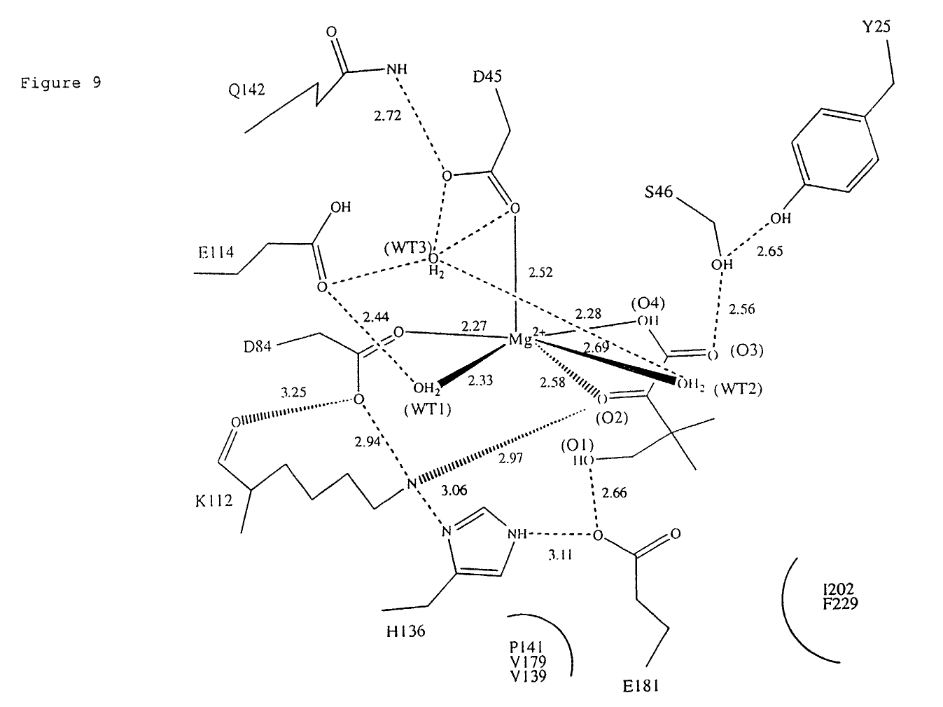

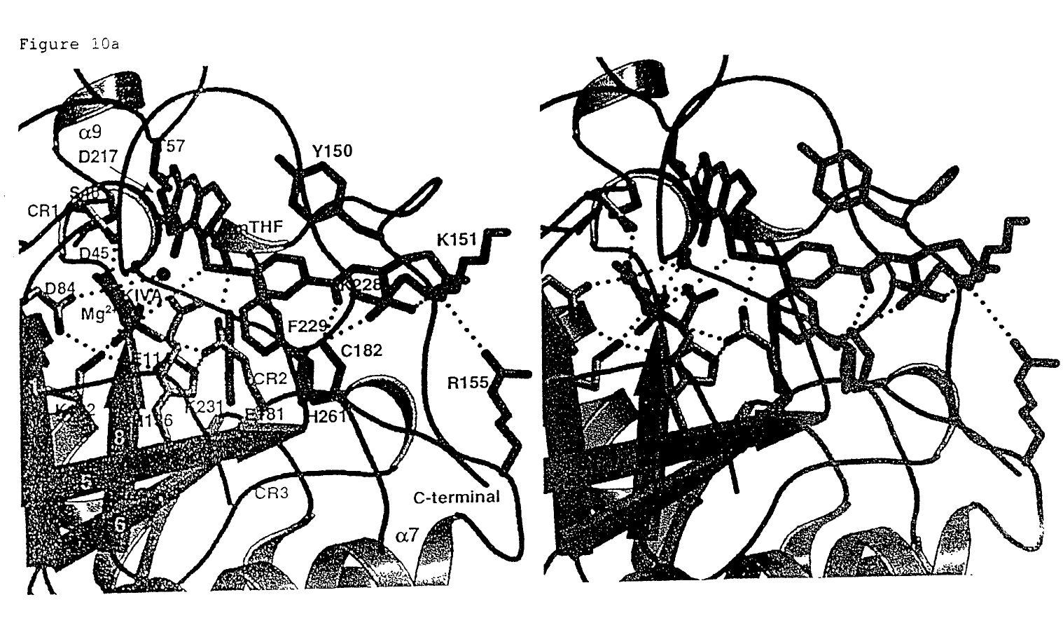

The Mg²⁺ ion is bound in a distorted octahedral binding site of the binding cavity. Residues, Asp 45 and Asp 84 occupy axial and equatorial positions, respectively, while Glu 114 coordinates to Mg²⁺ through a water molecule that occupies an equatorial position. The keto and carboxyl groups of the product take up an axial and an equatorial position, respectively and the last equatorial position is occupied by a water molecule. FIG. 9 shows a schematic representation of the distorted octahedral binding site.

The coordination around Mg²⁺ is distorted due to hydrogen bonding between Glu 181 and the hydroxymethyl group of the product. We believe the geometry of the Mg²⁺ ion is less distorted, and hence lower in stabilization energy, when ketopantoate (product) is replaced by α-KIVA (substrate). This may be one mechanism by which the enzyme senses and releases the product.

Cofactor Binding Site

As yet, a 5,10—CH₂—H₄folate cofactor binding motif has not been identified by X-ray crystallography. Nonetheless, we have developed an approach to find the cofactor binding site.

Initially we compared our structure to structures of tetrahydrofolate-dependent enzymes bound to folate analogues. The January, 2001 release of the Protein Data Bank (PDB) contains seven enzymes that bind tetrahydrofolate (THF). These are dihydrofolate reductase (DHFR), phosphoribosylglycinamide formyltransferase (PRGF), methylenetetrahydrofolate dehydrogenase (MTDH), glycinamide ribonucleotide transformylase (GRTF), thymidylate synthase (TS), serine hydroxymethyl transferase (SHMT), and methylenetetrahydrofolate reductase (MTR). A structural similarity search by the program DALI (24) shows that only four of the above proteins appear to be similar to KPHMT. These are MTR, DHFR, PRGF and SHMT, but for MTR, DHFR and PRGF, the distance of the folate cofactor binding site is too far from the substrate binding site relative to the corresponding distance in KPHMT.

This left SHMT, which appears to be functionally similar to KPHMT, although SHMT is a class I aldolase (KPHMT is a class II aldolase) because pyridoxal phosphate is used in addition to the folate cofactor. Given the crystal structures of SHMT from E. coli bound to the folate, 5-formyl-THF (25) and TS bound to 5,10—CH₂—H₄folate or analogues thereof (26), we were able to propose a tentative model for the binding 5,10—CH₂—H₄folate to KPHMT.

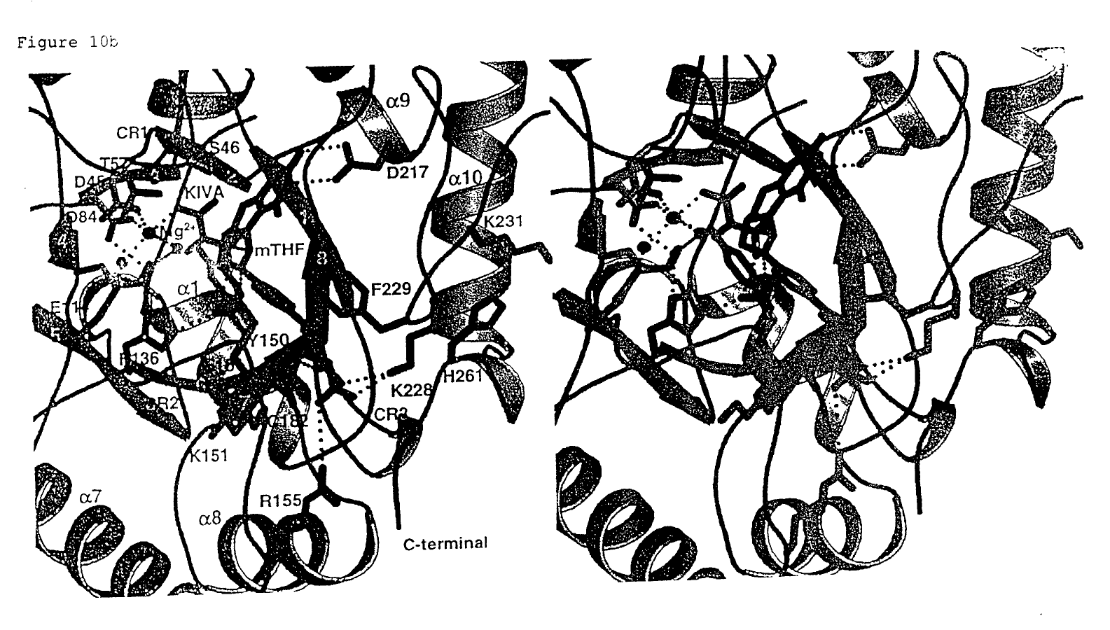

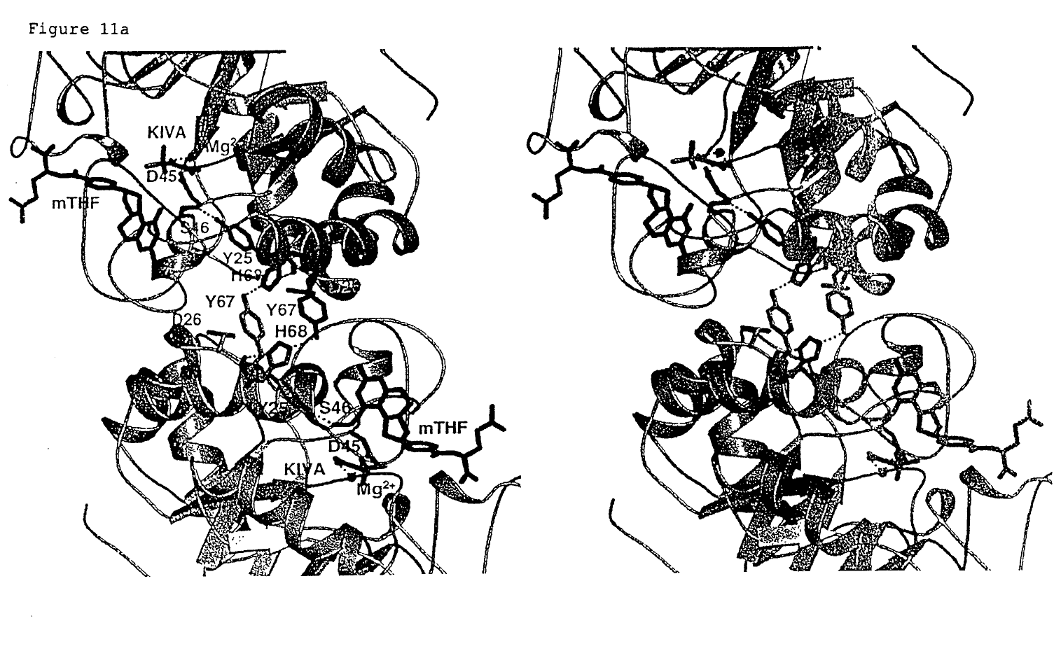

Next, using multiple sequence alignment (see FIG. 5) to identify residues implicated in cofactor binding, we were able to fine tune the proposed model for cofactor binding. The fine tuned model is shown in FIGS. 10a and b which are side and top view stereo pair ribbon representations of the mouth of the binding cavity.

In this model, 5,10—CH₂—H₄folate (mTHF) binds near the entrance to the binding cavity at a depth of 15 Å. The distance between the target carbon atoms, C11 in 5,10—CH₂—H₄folate and C3 in the substrate, is about 4.5 Å, a favourable distance for a reaction to occur.

The cofactor makes relatively few contacts with the protein. Interestingly, these contacts are located in regions of undefined secondary structure, namely, the loop regions that compose the entrance to the binding cavity. The loops in question are between β5 and α7 (L1), α9 and α10 (L2) and the C-terminus (L3). Being regions of undefined secondary structure these loops may be highly flexible and thus, undergo structural changes upon cofactor binding. We have identified conserved residues that impart either flexibility or make strong interactions that may impart rigidity (definition) to these loops. Thus we believe that upon cofactor binding these loops undergo discrete structural changes.

Loop, L1, contains two of the six above-mentioned conserved motifs. The first half of this loop, is located deeper in the binding cavity and contains Gln 142, which H-bonds to the axial Mg²⁺ ligand, Asp 45. This half of the loop is probably rigid since it contains a turn between Asn 145 and Gly 149. The second half of the loop consists predominantly of the second motif. Both ends of this motif, namely residues Gly 148 (invariant) and Gly 154 may make this part of the loop flexible. Gln 153 is implicated in a hydrogen bond to the amide of Lys 151, which upon cofactor binding may move to interact with the polyglutamate chain of the cofactor (see below for more discussion of this). Loop, L2, is relatively long with little sequence conservation. Invariant Gly 220 may impart some flexibility to this loop while residues Asp 217, Lys 228 and Phe 229 are implicated in binding the cofactor. In L3, invariant Pro 257 is in van der Waals contact with Gly 205 and Gln 211, while His 261 hydrogen bonds to Lys 228 and Glu 260. Thus, the deletion of Gly 168 in A. nidulans would lead to a distortion in the loop between β7 and β8 which may in turn lead to a disordering in adjacent loops such as L3 which could potentially prevent cofactor from binding. We, therefore propose that the panB auxotroph from A. nidulans is caused by the inability of the mutant KPHMT enzyme to bind the cofactor and therefore to function.

There are four main protein-cofactor interactions, namely, three hydrogen bonds and a n-stacking interaction. The nitrogen atom at N2 of 5,10—CH₂—H₄folate hydrogen bonds to Asp 217, while the side chain carboxyl group of the first glutamate hydrogen bonds to the carboxyl group of Tyr 150, and Lys 228. A stronger interaction is a n-stacking or hydrophobic interaction between the p-aminobenzoic acid (PABA) ring of the cofactor and the highly conserved residues Tyr 150 and Phe 229. Tyr 150 or phenylalanine, which in this instance is a functional replacement, is found at this position in 31 out of the 35 KPHMT sequences discussed above, while Phe 229 is found at this position in 34 out of the 35 KPHMT sequences. Interestingly, crystal structures of the THF-dependent enzymes, TS and SHMT, with cofactor analogues bound, also implicate a n-stacking or hydrophobic interaction between the PABA ring and a tyrosine or phenylalanine (25). It would appear that nature has converged on this mechanism to bind folate cofactors.

Most folate-dependent enzymes have a higher affinity for the polyglutamate form of the folate cofactor, with the greatest increase in affinity occurring with two or three glutamate residues (27). Presumably, the polyglutamate tail increases the affinity for enzyme through interactions with surface positive charges. In the crystal structure of the bifunctional enzyme dihydrofolate reductase-thymidylate synthase from Leishmania major, the polyglutamate tail of dihydrofolate makes few specific contacts but rather is held in place by the positive charge of the local electrostatic field (28). We have identified four positive residues in KPHMT that could interact with the polyglutamate tail. These are Lys 151, Arg 155 (in loop L1), Lys 231 (in loop L2) and His 261 (in loop L3).

KPHMT Catalysis

KPHMT catalyses the transfer of a hydroxymethyl group from cofactor (5,10—CH₂—H₄folate) to substrate (α-KIVA) The transferase reaction is an aldol reaction, namely deprotonation of the C3- carbon of α-KIVA followed by nucleophilic attack on the cofactor. The crystal structure of the apo enzyme gives insights in to the first stage in the enzyme mechanism, namely, activation of substrate and cofactor.

The C3 carbon is intrinsically acidic, through conjugation of the carboxyl and keto group, however, its acidity is enhanced by coordination of the substrate to the magnesium ion. Magnesium coordination also anchors and orients the substrate for subsequent deprotonation and nucleophilic attack. Also, the increase in distortion from octahedral geometry between substrate and product bound to the ion may be one mechanism by which the enzyme senses and releases product. The basic residue involved in abstraction of the C-3 proton of α-KIVA is believed to be Glu 181. The basicity of this residue is enhanced by a network of hydrogen bonds connecting residue Glu 181 with residues His 136 and Lys112, which constitute an invariant triad. In the crystal structure of the apo enzyme, Glu 181 is involved in a hydrogen bond with the hydroxymethyl group of the product ketopantoate—giving rise to the greater distortion from octahedral geometry. A final role for this versatile residue is as the acid in the protonation of N10 of 5,10—CH₂—H₄folate. Kallen and Jencks (29) have concluded that the reactive component of the 5,10—CH₂—H₄folate cofactor is the iminium intermediate, formed by breakage of the C11-N10-bond and protonation of N10. This is supported by the crystal structure of TS from Lactobacillus casei where the imidazolidine ring has opened and the iminium intermediate has been hydrated (26). Thus, Glu 181 is believed to abstract a proton from αKIVA and supply it to the cofactor.

Evidence for Cooperativity

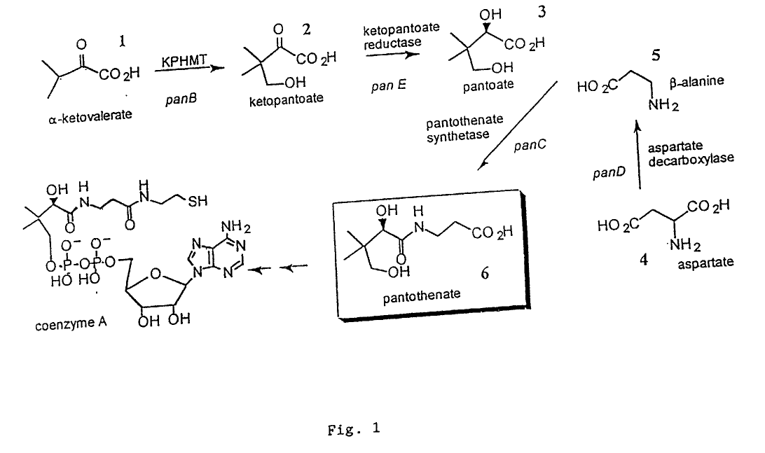

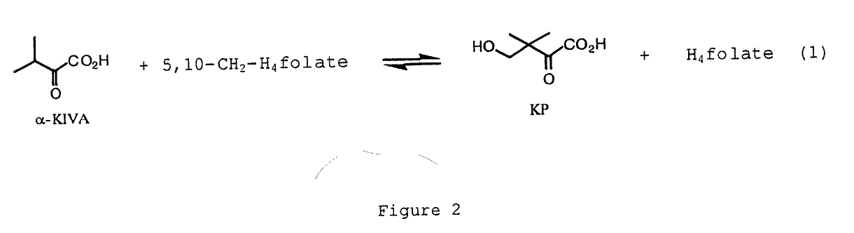

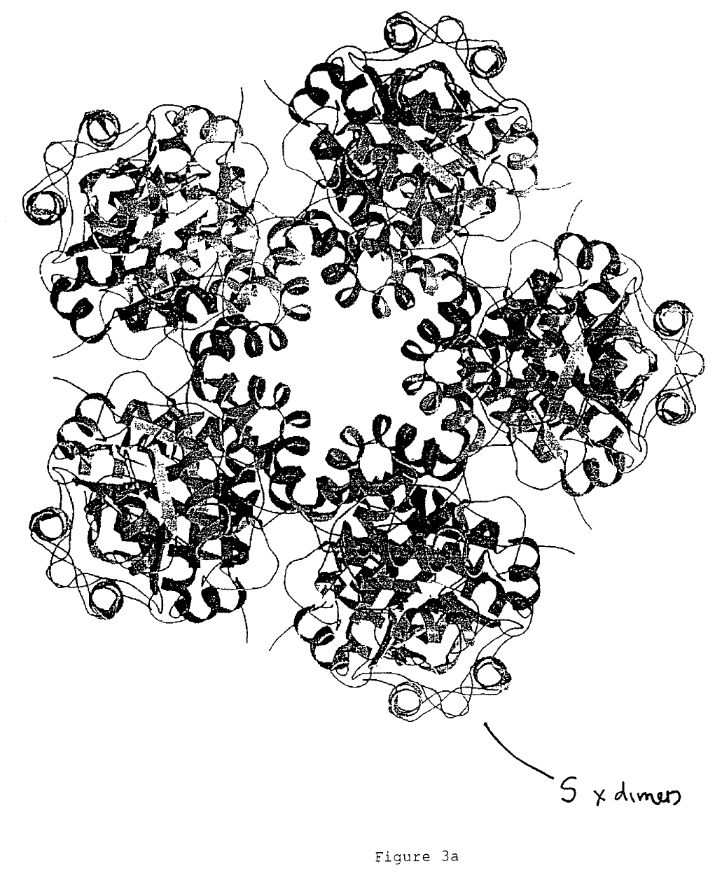

KPHMT, the first enzyme in the pathway for the synthesis of pantothenic acid (see FIG. 1), is inhibited by later intermediates, namely pantoate, pantothenate and CoA (1). This is most probably linked to the decameric architecture of the enzyme and involves multiple binding sites for effectors such as later pathway intermediates. All three, pantoate, pantothenate and CoA exhibit negative feedback, decreasing Vₘₐₓ, increasing Kₘ and enhancing cooperativity for the substrate. We believe we have found evidence, albeit tentative, of communication between protomers, a pre-requisite for cooperativity.

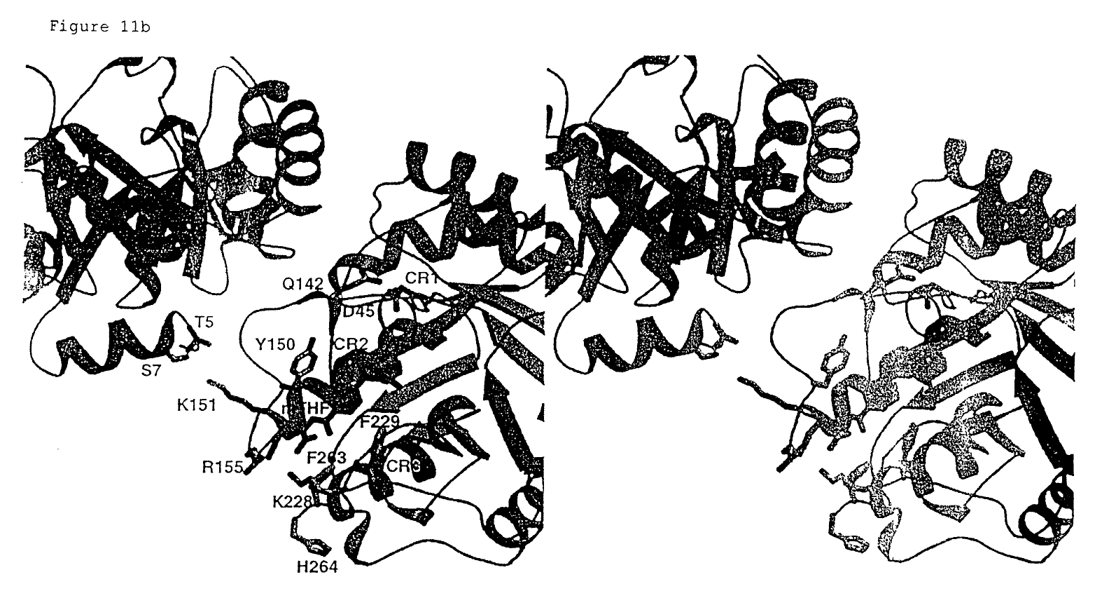

As pointed out earlier, the interface between protomers in the dimeric unit is tightly organised, involving 88 (20 hydrophilic and 68 hydrophobic) interactions. In particular one network of hydrogen bonds links the binding cavities of the vertically adjacent protomers. In the crystal structure of the apo enzyme, the products in the two vertically adjacent binding cavities are separated by only 31 Å. The H-bond network extends from ketopantoate to Ser 46 then Tyr 25 and His 68 of one subunit to Tyr 67 then His 68 then Tyr 25 then Ser 46 and finally ketopantoate of the next subunit. In the multiple sequence alignment discussed above all residues except Tyr 67 are conserved. An interaction between Asp 26 of one subunit and His 68 of the next could replace this interaction in organisms where there is no residue at position 67 able to H-bond to His 68. The alternate H-bond network would then extend from keptanoate to Ser 46 then Tyr 25 then Asp 26 of one subunit to His 68 then Tyr 25 then Ser 46 and finally keptanoate of the next subunit (see FIG. 11b which is a stereo pair ribbon representation of the binding cavities of a modified KPHMT dimer).

We believe we have also identified communication between subunits within the same pentamer. This interface is close to the opening to the binding cavity, the C-terminus (loop, L3), loop, L1 and the N-terminus of the adjacent subunit (see FIG. 11b which is a stereo pair ribbon representation of the interface between adjacent KPHMT dimers). Binding of cofactor and substrate would affect the structure of loops, L1 and L3 and thus affect the interaction at this interface. Of particular note, is the region within loop, Li consisting of residues Gly 138 to Glu 158. Residues, Gln 142 and Tyr 150 are respectively implicated in interacting with Mg²⁺ (indirectly) and cofactor. We have also identified a residue, Lys 151, that in the crystal structure of the apo enzyme H-bonds across the interface to Thr 5 of the adjacent dimer. We speculate that binding of cofactor will cause loop L1 to move in this region, the Lys 151-Thr 5 interaction to break, and a new interaction between Lys 151 and the polyglutamate tail of the cofactor to form.

Structure-Based Drug Design

Determination of the 3D structure of KPHMT provides important information about the binding sites of KPHMT, particularly when comparisons are made with similar enzymes. This information may then be used for rational design of KPHMT inhibitors, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Greer et al. describes an iterative approach to ligand design based on repeated sequences of computer modelling, protein-ligand complex formation and X-ray crystallographic or NMR spectroscopic analysis. Thus novel thymidylate synthase inhibitor series were designed de novo by Greer et al., and KPHMT inhibitors may also be designed in the this way. More specifically, using e.g. GRID on the solved 3D structure of KPHMT, a ligand (e.g. a potential inhibitor) for KPHMT may be designed that complements the functionalities of the KPHMT binding site(s). The ligand can then be synthesised, formed into a complex with KPHMT, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek et al., Medicinal Research Reviews, Vol.16, (1996), 3-50.

As a result of the determination of the KPHMT 3D structure, more purely computational techniques for rational drug design may also be used to design KPHMT inhibitors (for an overview of these techniques see e.g. Walters et al.) . For example, automated ligand-receptor docking programs (discussed e.g. by Jones et al. in Current Opinion in Biotechnology, Vol.6, (1995), 652-656) which require accurate information on the atomic coordinates of target receptors may be used to design potential KPHMT inhibitors.

Linked-fragment approaches to drug design also require accurate information on the atomic coordinates of target receptors. The basic idea behind these approaches is to determine (computationally or experimentally) the binding locations of plural ligands to a target molecule, and then construct a molecular scaffold to connect the ligands together in such a way that their relative binding positions are preserved. The connected ligands thus form a potential lead compound that can be further refined using e.g. the iterative technique of Greer et al.. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design KPHMT inhibitors is made possible by the determination of the KPHMT structure.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al.) it is necessary to know beforehand the protein 3D structure (or at least the protein structure factors). Therefore, determination of the KPHMT structure also allows difference Fourier electron density maps of KPHMT-ligand complexes to be produced, which can greatly assist the process of rational drug design.

The approaches to structure-based drug design described above all require initial identification of possible compounds for interaction with target bio-molecule (in this case KPHMT). Sometimes these compounds are known e.g. from the research literature. However, when they are not, or when novel compounds are wanted, a first stage of the drug design program may involve computer-based in silica screening of compound databases (such as the Cambridge Structural Database) with the aim of identifying compounds which interact with the binding site or sites of the target bio-molecule. Screening selection criteria may be based on pharmacokinetic properties such as metabolic stability and toxicity. However, determination of the KPHMT structure allows the architecture and chemical nature of each KPHMT binding site to be identified, which in turn allows the geometric and functional constraints of a descriptor for the potential inhibitor to be derived. The descriptor is, therefore, a type of virtual 3-D pharmacophore, which can also be used as selection criteria or filter for database screening.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention.

The references in the above text and listed below are incorporated by reference.

### REFERENCES

1) Powers, S. G. & Snell, E. E. (1976). Ketopantoate Hydroxymethyltransferase (II) [Physical, Catalytic, and Regulatory Properties]. J. Biol. Chem. 251, 3786-3793.

2) Jones, C. E., Brook, J., Buck, D., Abell, C. and Smith, A. G. (1993). Cloning and sequencing of the Escherichia coli panB gene, which encodes ketopantoate hydroxymethyltransferase, and overexpression of the enzyme. Journal of Bacteriology 175, 2125-2130.

3) Kurtov, D., Kinghorn, J. R., and Unkles, S. E. (1999). The Aspergillus nidulans panB gene encodes ketopantoate hydroxymethyltransferase, required for biosynthesis of pantothenate and Coenzyme A. Mol. Gen. Genet. 262, 115-120.

4) Aberhart, D. J. and Russel, D. J. (1984). Steric course of N5,N10-methylenetetrahydrofolate formation from glycine by the glycine cleavage system in Escherichia coli. Journal of the American Chemical Society 106, 4907-4910.

5) Cronan Jr, J E, Littel, K J and Jackowski, S (1982). Genetic and biochemical analyses of pantothenate biosyntheis in Escherichia coli and Salmonella typhimurium. J. Bacteriol. 149, 916-922.

6) Sahi, S. V., Saxena, P. K., Abrahams, G. D. and King, J. (1988). Identification of the biochemical lesion in a pantothenate-requiring auxotroph of Datura innoxia P. Mill J. Plant Physiol., 133, 277-280

7) Brûnger, A. T., Kurian, J., and Karplus, M. (1987). Crystallographic R factor refinement by molecular dynamics. Science 235, 458-560.

8) Doublie, S. (1997). Preparation of Selenomethyionyl Proteins for Phase Determination. Methods in Enzymology 276, 523-530.

9) Hendrickson W. A. (1991). Determination of macromolecular structures from anomalous diffraction of synchrotron radiation. Science 254, 51-58.

10) Pflugrath, J. W. (1999). The finer things in X-ray diffraction data collection. Acta Crystallographica D55, 1718-1725.

11) Leslie, A. G. W. (1992). In Joint CCP4 and EESF-EACMB Newsletter on Protein Crystallography, vol. 26, Warrington, Daresbury Laboratory.

12) CCP4—Collaborative Computational Project 4. (1994) The CCP4 Suite: Programs for Protein Crystallography. Acta Crystallographica D50, 760-763.

13) Evans, P. R. (1997). Scaling of MAD data. In Recent Advances in Phasing (ed. K. S. Wilson, G. Davies, A. W. Ashton and S. Bailey), pp. 97-102. Council for the Central Laboratory of the Research Councils Daresbury Laboratory, Daresbury, UK.

14) Weeks, C. M., Detitta, G. T., Hauptman, H. A., Thuman, P. and Miller, R. (1994). Structure solution by minimal function phase refinement and Fourier filtering II. Implementation and applications. Acta Crystallographica A50, 210-220.

15) La Fortelle, E. de and Bricogne, G. (1997). Maximum-likelihood heavy-atom parameter refinement for multiple isomorphous replacement and multiwavelength anomalous diffraction methods. Methods in Enzymology 276, 472-494.

16) Jones, T. A., Zou, J. Y., Cowan, S. W. and Kjeldgaard, M. (1991). Improved methods for building protein models in electron density maps and the location of errors in these models. Acta Crystallographica, A47, 110-119.

17) Murshudov, G. N., Vagin, A. A. and Dodson, E. J. (1997). Refinement of macromolecular structures by the maximum-likelihood method. Acta Crystallographica, 1997 D53, 240-255.

18) Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. and Warren, G. L. (1998). Crystallography & NMR system: A new software suite for macromolecular structure determination. Acta Crystallographica D54, 905-921.

19) Laskowski, R. A. (1993). PROCHECK: a program to check the stereochemical quality of protein structures. J. Appl. Cryst. 26, 283-291.

20) Teller, J. H., Powers, S. G. and Snell, E. E. (1976). Ketopantoate hydroxymethyltransferase. I. Purification and role in pantothenate biosynthesis. J. Biol. Chem. 251, 3780-3785.

21) Brândén, C.-I. (1980). Relation between structure and function of alpha-beta-proteins. Quarterly Rev. of Biophys. 13, 317-338.

22) Altschul, S. F., Madden, T. L., Schaffer, A. A., Zhang, Z., Miller, W. and Lipman, D. J. (1997). Gapped BLAST and PSI-BLAST: a new generation of protein database search programs. Nucleic Acids Res., 25 3389-3402.

23) Thompson, J. D., Higgins, D. G. and Gibson, T. J. (1994). CLUSTAL W: improving the sensitivity of progressive multiple sequence alignment through sequence weighting, positions-specific gap penalties and weight matrix choice. Nucleic Acids Research 22, 4673-4680.

24) Holm, L. and Sander, C. (1998). Touring protein fold space with Dali/FSSP. Nucleic Acids Research 26, 316-319.

25) Scarsdale, J. N., Kazanina, G., Radaev, S., Schirch, V., and Wright, H. T. (1999). Crystal structure of rabbit cytosolic serine hydroxymethyltransferase at 2.8 Å resolution: mechanistic implications. Biochemistry 38, 8347-8358.

26) Perry, K. M., Fauman, E. B., Finer-Moore, J. S., Montfort, W. R., Maley, G. F., Maley, F., Stroud, R. M. (1990). Plastic adaptation toward mutations in proteins: structural comparison of thymidylate syntheses. Proteins 8, 315-333.

27) Schirch, V., and Strong, W. B. (1989) Interaction of folylpolyglutamates with enzymes in one-carbon metabolism. Arch. Biochem. Biophys. 269, 371-380.

28) Knighton, D. R., Kan, C. C., Howland, E., Janson, C. A., Hostomska, Z., Welsh, K. M. and Matthews, D. A. (1994). Structure of and kinetic channelling in bifunctional dihydrofolate reductase-thymidylate synthase. Nat. Struct. Biol. 1, 186-194.

29) Kallen, R. G. & Jencks. (1966). The mechanism of the condensation of formaldehyde with tetrahydrofolic acid. Journal of Biological Chemistry 241, 5851-5863.

TABLE 1
REMARK coordinates from minimization and B-factor refinement
REMARK refinement resolution: 500.0 − 1.8 A
REMARK starting r = 0.2289 free_r = 0.2635
REMARK final r = 0.2292 free_r = 0.2638
REMARK rmsd bonds = 0.005641 rmsd angles = 1.11562
REMARK B rmsd for bonded mainchain atoms = 1.325 target = 1.5
REMARK B rmsd for bonded sidechain atoms = 2.001 target = 2.0
REMARK B rmsd for angle mainchain atoms = 2.071 target = 2.0
REMARK B rmsd for angle sidechain atoms = 2.863 target = 2.5
REMARK target = mlf final wa = 1.10321
REMARK final rweight = 0.0678 (with wa = 1.10321)
REMARK md-method = torsion annealing schedule = constant
REMARK starting temperature = 1000 total md steps = 1 * 100
REMARK cycles = 2 coordinate steps = 20 B-factor steps = 10
REMARK sg = P2(1) a = 86.074 b = 157.17 c = 100.181 alpha = 90 beta = 97.44 gamma = 90
REMARK topology file 1: CNS_TOPPAR: protein.top
REMARK topology file 2: CNS_TOPPAR: dna-rna.top
REMARK topology file 3: CNS_TOPPAR: water.top
REMARK topology file 4: CNS_TOPPAR: ion.top
REMARK topology file 5: ./TOPH_PARAM/kpl.toph
REMARK parameter file 1: CNS_TOPPAR: protein_rep.param
REMARK parameter file 2: CNS_TOPPAR: dna-rna_rep.param
REMARK parameter file 3: CNS_TOPPAR: water_rep.param
REMARK parameter file 4: CNS_TOPPAR: ion.param
REMARK parameter file 5: ./TOPH_PARAM/kpl.param
REMARK molecular structure file: generate.mtf
REMARK input coordinates: generate.pdb
REMARK reflection file = ./int/panb.cv
REMARK ncs = none
REMARK B-correction resolution: 6.0 − 1.8
REMARK initial B-factor correction applied to fobs:
REMARK B11 = −1.301 B22 = −2.124 B33 = 3.425
REMARK B12 = 0.000 B13 = 1.230 B23 = 0.000
REMARK B-factor correction applied to coordinate array B: 0.254
REMARK bulk solvent: density level = 0.392735 e/A\hat{ }3, B-factor = 64.4356 A\hat{ }2
REMARK reflections with |Fobs|/sigma_F < 0.0 rejected
REMARK reflections with |Fobs| > 10000 * rms(Fobs) rejected
REMARK theoretical total number of refl. in resol. range: 243384 (100.0%)
REMARK number of unobserved reflections (no entry or |F| = 0): 14308 (5.9%)
REMARK number of reflections rejected: 0 (0.0%)
REMARK total number of reflections used: 229076 (94.1%)
REMARK number of reflections in working set: 206168 (84.7%)
REMARK number of reflections in test set: 22908 (9.4%)
CRYST1 86.074 157.170 100.181 90.00 97.44 90.00 P 21
REMARK FILENAME = “refine.pdb”
REMARK DATE: 17-Oct-00 01:40:10 created by user: inouet
REMARK VERSION: 1.0

| | | | | | | | | | |
|---|---|---|---|---|---|---|---|---|---|
| ATOM | 1 | CB | MET | 1 | 1.201 | 12.262 | 69.884 | 1.00 | 67.43 |
| ATOM | 2 | CG | MET | 1 | 0.767 | 11.220 | 70.906 | 1.00 | 69.43 |
| ATOM | 3 | SD | MET | 1 | 1.582 | 11.428 | 72.507 | 1.00 | 72.24 |
| ATOM | 4 | CE | MET | 1 | 3.012 | 10.336 | 72.306 | 1.00 | 71.04 |
| ATOM | 5 | C | MET | 1 | 1.282 | 10.813 | 67.848 | 1.00 | 63.63 |
| ATOM | 6 | O | MET | 1 | 2.165 | 10.936 | 66.998 | 1.00 | 63.58 |
| ATOM | 7 | N | MET | 1 | −0.854 | 11.909 | 68.546 | 1.00 | 65.98 |
| ATOM | 8 | CA | MET | 1 | 0.631 | 12.042 | 68.480 | 1.00 | 65.57 |
| ATOM | 9 | N | LYS | 2 | 0.841 | 9.631 | 68.271 | 1.00 | 61.39 |
| ATOM | 10 | CA | LYS | 2 | 1.376 | 8.379 | 67.750 | 1.00 | 58.18 |
| ATOM | 11 | CB | LYS | 2 | 1.946 | 7.518 | 68.886 | 1.00 | 59.31 |
| ATOM | 12 | CG | LYS | 2 | 3.141 | 8.121 | 69.610 | 1.00 | 60.51 |
| ATOM | 13 | CD | LYS | 2 | 3.805 | 7.096 | 70.523 | 1.00 | 61.39 |
| ATOM | 14 | CE | LYS | 2 | 2.844 | 6.572 | 71.585 | 1.00 | 62.31 |
| ATOM | 15 | NZ | LYS | 2 | 3.441 | 5.457 | 72.377 | 1.00 | 62.32 |
| ATOM | 16 | C | LYS | 2 | 0.313 | 7.577 | 67.003 | 1.00 | 54.66 |
| ATOM | 17 | O | LYS | 2 | −0.258 | 6.631 | 67.548 | 1.00 | 55.68 |
| ATOM | 18 | N | PRO | 3 | 0.021 | 7.953 | 65.749 | 1.00 | 50.09 |
| ATOM | 19 | CD | PRO | 3 | −0.580 | 7.007 | 64.792 | 1.00 | 49.24 |
| ATOM | 20 | CA | PRO | 3 | 0.633 | 9.074 | 65.034 | 1.00 | 45.89 |
| ATOM | 21 | CB | PRO | 3 | 0.847 | 8.501 | 63.644 | 1.00 | 47.20 |
| ATOM | 22 | CG | PRO | 3 | −0.403 | 7.717 | 63.455 | 1.00 | 47.74 |
| ATOM | 23 | C | PRO | 3 | −0.291 | 10.290 | 65.014 | 1.00 | 41.96 |
| ATOM | 24 | O | PRO | 3 | −1.403 | 10.253 | 65.547 | 1.00 | 40.18 |
| ATOM | 25 | N | THR | 4 | 0.175 | 11.363 | 64.389 | 1.00 | 37.27 |
| ATOM | 26 | CA | THR | 4 | −0.605 | 12.586 | 64.284 | 1.00 | 33.03 |
| ATOM | 27 | CB | THR | 4 | 0.316 | 13.808 | 64.214 | 1.00 | 32.00 |
| ATOM | 28 | OG1 | THR | 4 | 1.113 | 13.866 | 65.403 | 1.00 | 29.60 |
| ATOM | 29 | CG2 | THR | 4 | −0.496 | 15.084 | 64.077 | 1.00 | 30.09 |
| ATOM | 30 | C | THR | 4 | −1.436 | 12.516 | 63.012 | 1.00 | 31.90 |
| ATOM | 31 | O | THR | 4 | −0.890 | 12.415 | 61.917 | 1.00 | 31.80 |
| ATOM | 32 | N | THR | 5 | −2.755 | 12.574 | 63.156 | 1.00 | 30.88 |
| ATOM | 33 | CA | THR | 5 | −3.636 | 12.494 | 61.999 | 1.00 | 29.95 |
| ATOM | 34 | CB | THR | 5 | −4.616 | 11.320 | 62.137 | 1.00 | 30.13 |
| ATOM | 35 | OG1 | THR. | 5 | −5.545 | 11.602 | 63.189 | 1.00 | 31.18 |
| ATOM | 36 | CG2 | THR | 5 | −3.864 | 10.035 | 62.462 | 1.00 | 29.84 |
| ATOM | 37 | C | THR | 5 | −4.445 | 13.764 | 61.789 | 1.00 | 28.70 |
| ATOM | 38 | O | THR | 5 | −4.407 | 14.684 | 62.605 | 1.00 | 28.11 |
| ATOM | 39 | N | ILE | 6 | −5.184 | 13.804 | 60.685 | 1.00 | 28.91 |
| ATOM | 40 | CA | ILE | 6 | −6.009 | 14.961 | 60.360 | 1.00 | 29.62 |
| ATOM | 41 | CB | ILE | 6 | −6.777 | 14.749 | 59.042 | 1.00 | 30.80 |
| ATOM | 42 | CG2 | ILE | 6 | −7.445 | 16.047 | 58.617 | 1.00 | 29.12 |
| ATOM | 43 | CG1 | ILE | 6 | −5.813 | 14.298 | 57.945 | 1.00 | 33.13 |
| ATOM | 44 | CD1 | ILE | 6 | −6.513 | 13.840 | 56.671 | 1.00 | 34.94 |
| ATOM | 45 | C | ILE | 6 | −7.016 | 15.189 | 61.477 | 1.00 | 29.79 |
| ATOM | 46 | O | ILE | 6 | −7.339 | 16.327 | 61.813 | 1.00 | 30.34 |
| ATOM | 47 | N | SER | 7 | −7.499 | 14.091 | 62.051 | 1.00 | 29.76 |
| ATOM | 48 | CA | SER | 7 | −8.474 | 14.142 | 63.138 | 1.00 | 30.44 |
| ATOM | 49 | CB | SER | 7 | −8.748 | 12.730 | 63.653 | 1.00 | 31.47 |
| ATOM | 50 | OG | SER | 7 | −8.920 | 11.822 | 62.575 | 1.00 | 37.31 |
| ATOM | 51 | C | SER | 7 | −7.954 | 15.006 | 64.285 | 1.00 | 29.13 |
| ATOM | 52 | O | SER | 7 | −8.712 | 15.751 | 64.909 | 1.00 | 28.63 |
| ATOM | 53 | N | LEU | 8 | −6.655 | 14.902 | 64.556 | 1.00 | 27.78 |
| ATOM | 54 | CA | LEU | 8 | −6.035 | 15.668 | 65.630 | 1.00 | 27.46 |
| ATOM | 55 | CB | LEU | 8 | −4.553 | 15.296 | 65.778 | 1.00 | 28.11 |
| ATOM | 56 | CG | LEU | 8 | −3.954 | 15.254 | 67.190 | 1.00 | 30.83 |
| ATOM | 57 | CD1 | LEU | 8 | −2.452 | 15.499 | 67.105 | 1.00 | 30.15 |
| ATOM | 58 | CD2 | LEU | 8 | −4.594 | 16.302 | 68.086 | 1.00 | 32.65 |
| ATOM | 59 | C | LEU | 8 | −6.141 | 17.173 | 65.378 | 1.00 | 26.61 |
| ATOM | 60 | O | LEU | 8 | −6.388 | 17.943 | 66.303 | 1.00 | 26.41 |
| ATOM | 61 | N | LEU | 9 | −5.951 | 17.589 | 64.129 | 1.00 | 23.76 |
| ATOM | 62 | CA | LEU | 9 | −6.024 | 19.005 | 63.799 | 1.00 | 24.65 |
| ATOM | 63 | CB | LEU | 9 | −5.388 | 19.279 | 62.431 | 1.00 | 22.20 |
| ATOM | 64 | CG | LEU | 9 | −3.926 | 18.838 | 62.239 | 1.00 | 19.92 |
| ATOM | 65 | CD1 | LEU | 9 | −3.403 | 19.371 | 60.914 | 1.00 | 18.82 |
| ATOM | 66 | CD2 | LEU | 9 | −3.076 | 19.367 | 63.382 | 1.00 | 18.44 |
| ATOM | 67 | C | LEU | 9 | −7.468 | 19.500 | 63.805 | 1.00 | 26.68 |
| ATOM | 68 | O | LEU | 9 | −7.737 | 20.650 | 64.151 | 1.00 | 26.46 |
| ATOM | 69 | N | GLN | 10 | −8.396 | 18.627 | 63.426 | 1.00 | 29.15 |
| ATOM | 70 | CA | CLN | 10 | −9.808 | 18.990 | 63.403 | 1.00 | 32.76 |
| ATOM | 71 | CB | GLN | 10 | −10.632 | 17.869 | 62.764 | 1.00 | 32.40 |
| ATOM | 72 | CG | GLN | 10 | −12.091 | 18.220 | 62.511 | 1.00 | 34.98 |
| ATOM | 73 | CD | GLN | 10 | −12.263 | 19.495 | 61.698 | 1.00 | 34.51 |
| ATOM | 74 | OE1 | GLN | 10 | −12.158 | 20.601 | 62.228 | 1.00 | 35.90 |
| ATOM | 75 | NE2 | GLN | 10 | −12.518 | 19.343 | 60.403 | 1.00 | 33.91 |
| ATOM | 76 | C | GLN | 10 | −10.256 | 19.239 | 64.841 | 1.00 | 34.38 |
| ATOM | 77 | O | GLN | 10 | −11.132 | 20.066 | 65.093 | 1.00 | 35.73 |
| ATOM | 78 | N | LYS | 11 | −9.640 | 18.528 | 65.781 | 1.00 | 36.20 |
| ATOM | 79 | CA | LYS | 11 | −9.961 | 18.694 | 67.193 | 1.00 | 37.50 |
| ATOM | 80 | CB | LYS | 11 | −9.374 | 17.548 | 68.023 | 1.00 | 39.71 |
| ATOM | 81 | CG | LYS | 11 | −9.466 | 17.788 | 69.526 | 1.00 | 42.23 |
| ATOM | 82 | CD | LYS | 11 | −8.571 | 16.850 | 70.326 | 1.00 | 45.49 |
| ATOM | 83 | CE | LYS | 11 | −9.131 | 15.436 | 70.387 | 1.00 | 47.75 |
| ATOM | 84 | NZ | LYS | 11 | −8.313 | 14.563 | 71.283 | 1.00 | 48.44 |
| ATOM | 85 | C | LYS | 11 | −9.378 | 20.016 | 67.677 | 1.00 | 37.17 |
| ATOM | 86 | O | LYS | 11 | −9.988 | 20.721 | 68.483 | 1.00 | 38.57 |
| ATOM | 87 | N | TYR | 12 | −8.189 | 20.345 | 67.181 | 1.00 | 36.59 |
| ATOM | 88 | CA | TYR | 12 | −7.512 | 21.585 | 67.548 | 1.00 | 35.53 |
| ATOM | 89 | CB | TYR | 12 | −6.145 | 21.665 | 66.864 | 1.00 | 36.51 |
| ATOM | 90 | CG | TYR | 12 | −5.070 | 20.803 | 67.486 | 1.00 | 37.30 |
| ATOM | 91 | CD1 | TYR | 12 | −3.889 | 20.534 | 66.797 | 1.00 | 38.03 |
| ATOM | 92 | CE1 | TYR | 12 | −2.880 | 19.769 | 67.371 | 1.00 | 38.86 |
| ATOM | 93 | CD2 | TYR | 12 | −5.217 | 20.281 | 68.772 | 1.00 | 37.36 |
| ATOM | 94 | CE2 | TYR | 12 | −4.213 | 19.515 | 69.356 | 1.00 | 38.58 |
| ATOM | 95 | CZ | TYR | 12 | −3.047 | 19.264 | 68.648 | 1.00 | 39.25 |
| ATOM | 96 | OH | TYR | 12 | −2.044 | 18.514 | 69.220 | 1.00 | 40.19 |
| ATOM | 97 | C | TYR | 12 | −8.324 | 22.815 | 67.169 | 1.00 | 34.82 |
| ATOM | 98 | O | TYR | 12 | −8.451 | 23.748 | 67.960 | 1.00 | 34.13 |
| ATOM | 99 | N | LYS | 13 | −8.860 | 22.822 | 65.953 | 1.00 | 34.10 |
| ATOM | 100 | CA | LYS | 13 | −9.652 | 23.960 | 65.496 | 1.00 | 35.74 |
| ATOM | 101 | CB | LYS | 13 | −10.087 | 23.765 | 64.041 | 1.00 | 34.52 |
| ATOM | 102 | CG | LYS | 13 | −10.895 | 24.927 | 63.458 | 1.00 | 34.21 |
| ATOM | 103 | CD | LYS | 13 | −11.268 | 24.645 | 62.001 | 1.00 | 31.63 |
| ATOM | 104 | CE | LYS | 13 | −12.274 | 25.645 | 61.433 | 1.00 | 31.85 |
| ATOM | 105 | NZ | LYS | 13 | −11.675 | 26.929 | 60.984 | 1.00 | 30.98 |
| ATOM | 106 | C | LYS | 13 | −10.878 | 24.124 | 66.385 | 1.00 | 36.28 |
| ATOM | 107 | O | LYS | 13 | −11.336 | 25.240 | 66.622 | 1.00 | 34.68 |
| ATOM | 108 | N | GLN | 14 | −11.404 | 23.004 | 66.869 | 1.00 | 38.20 |
| ATOM | 109 | CA | GLN | 14 | −12.572 | 23.018 | 67.744 | 1.00 | 40.77 |
| ATOM | 110 | CB | GLN | 14 | −13.049 | 21.591 | 68.007 | 1.00 | 42.50 |
| ATOM | 111 | CG | GLN | 14 | −13.662 | 20.906 | 66.800 | 1.00 | 47.11 |
| ATOM | 112 | CD | GLN | 14 | −13.789 | 19.407 | 66.992 | 1.00 | 49.44 |
| ATOM | 113 | OE1 | GLN | 14 | −14.221 | 18.939 | 68.046 | 1.00 | 51.52 |
| ATOM | 114 | NE2 | GLN | 14 | −13.419 | 18.645 | 65.967 | 1.00 | 50.79 |
| ATOM | 115 | C | GLN | 14 | −12.227 | 23.688 | 69.071 | 1.00 | 40.73 |
| ATOM | 116 | O | GLN | 14 | −13.043 | 24.409 | 69.648 | 1.00 | 40.91 |
| ATOM | 117 | N | GLU | 15 | −11.010 | 23.443 | 69.545 | 1.00 | 39.66 |
| ATOM | 118 | CA | GLU | 15 | −10.544 | 24.008 | 70.805 | 1.00 | 39.32 |
| ATOM | 119 | CB | GLU | 15 | −9.544 | 23.054 | 71.465 | 1.00 | 40.94 |
| ATOM | 120 | CG | GLU | 15 | −10.012 | 21.607 | 71.509 | 1.00 | 43.66 |
| ATOM | 121 | CD | GLU | 15 | −9.013 | 20.679 | 72.180 | 1.00 | 44.96 |
| ATOM | 122 | OE1 | GLU | 15 | −7.834 | 20.658 | 71.768 | 1.00 | 44.85 |
| ATOM | 123 | OE2 | GLU | 15 | −9.409 | 19.959 | 73.121 | 1.00 | 48.88 |
| ATOM | 124 | C | GLU | 15 | −9.880 | 25.357 | 70.567 | 1.00 | 38.67 |
| ATOM | 125 | O | GLU | 15 | −9.381 | 25.985 | 71.502 | 1.00 | 38.21 |
| ATOM | 126 | N | LYS | 16 | −9.889 | 25.803 | 69.313 | 1.00 | 37.45 |
| ATOM | 127 | CA | LYS | 16 | −9.269 | 27.069 | 68.939 | 1.00 | 36.93 |
| ATOM | 128 | CB | LYS | 16 | −9.957 | 28.235 | 69.655 | 1.00 | 39.24 |
| ATOM | 129 | CG | LYS | 16 | −10.820 | 29.105 | 68.748 | 1.00 | 42.46 |
| ATOM | 130 | CD | LYS | 16 | −9.963 | 29.912 | 67.783 | 1.00 | 44.77 |
| ATOM | 131 | CE | LYS | 16 | −10.809 | 30.700 | 66.794 | 1.00 | 45.69 |
| ATOM | 132 | NZ | LYS | 16 | −11.734 | 31.653 | 67.466 | 1.00 | 45.98 |
| ATOM | 133 | C | LYS | 16 | −7.777 | 27.055 | 69.272 | 1.00 | 35.34 |
| ATOM | 134 | O | LYS | 16 | −7.170 | 28.103 | 69.507 | 1.00 | 34.70 |
| ATOM | 135 | N | LYS | 17 | −7.186 | 25.865 | 69.304 | 1.00 | 33.29 |
| ATOM | 136 | CA | LYS | 17 | −5.759 | 25.752 | 69.593 | 1.00 | 32.28 |
| ATOM | 137 | CB | LYS | 17 | −5.440 | 24.423 | 70.285 | 1.00 | 34.01 |
| ATOM | 138 | CG | LYS | 17 | −3.951 | 24.238 | 70.580 | 1.00 | 36.37 |
| ATOM | 139 | CD | LYS | 17 | −3.618 | 22.820 | 71.033 | 1.00 | 39.39 |
| ATOM | 140 | CE | LYS | 17 | −4.198 | 22.504 | 72.405 | 1.00 | 41.80 |
| ATOM | 141 | NZ | LYS | 17 | −3.935 | 21.089 | 72.804 | 1.00 | 42.95 |
| ATOM | 142 | C | LYS | 17 | −4.955 | 25.854 | 68.298 | 1.00 | 30.45 |
| ATOM | 143 | O | LYS | 17 | −4.935 | 24.923 | 67.495 | 1.00 | 29.95 |
| ATOM | 144 | N | ARG | 18 | −4.299 | 26.993 | 68.103 | 1.00 | 28.24 |
| ATOM | 145 | CA | ARG | 18 | −3.486 | 27.224 | 66.913 | 1.00 | 27.71 |
| ATOM | 146 | CB | ARG | 18 | −3.084 | 28.693 | 66.841 | 1.00 | 26.93 |
| ATOM | 147 | CG | ARG | 18 | −4.213 | 29.588 | 66.366 | 1.00 | 29.98 |
| ATOM | 148 | CD | ARG | 18 | −3.904 | 31.058 | 66.576 | 1.00 | 31.56 |
| ATOM | 149 | NE | ARG | 18 | −3.975 | 31.427 | 67.989 | 1.00 | 33.11 |
| ATOM | 150 | CZ | ARG | 18 | −3.874 | 32.673 | 68.437 | 1.00 | 34.92 |
| ATOM | 151 | NH1 | ARG | 18 | −3.694 | 33.671 | 67.580 | 1.00 | 34.93 |
| ATOM | 152 | NH2 | ARG | 18 | −3.961 | 32.921 | 69.736 | 1.00 | 33.10 |
| ATOM | 153 | C | ARG | 18 | −2.249 | 26.329 | 66.912 | 1.00 | 26.09 |
| ATOM | 154 | O | ARG | 18 | −1.455 | 26.357 | 67.852 | 1.00 | 27.27 |
| ATOM | 155 | N | PHE | 19 | −2.093 | 25.546 | 65.845 | 1.00 | 24.65 |
| ATOM | 156 | CA | PHE | 19 | −0.983 | 24.601 | 65.710 | 1.00 | 21.83 |
| ATOM | 157 | CB | PHE | 19 | −1.543 | 23.213 | 65.386 | 1.00 | 22.18 |
| ATOM | 158 | CG | PHE | 19 | −2.394 | 23.176 | 64.147 | 1.00 | 21.17 |
| ATOM | 159 | CD1 | PHE | 19 | −1.813 | 23.054 | 62.885 | 1.00 | 21.71 |
| ATOM | 160 | CD2 | PHE | 19 | −3.779 | 23.275 | 64.240 | 1.00 | 21.90 |
| ATOM | 161 | CE1 | PHE | 19 | −2.604 | 23.036 | 61.730 | 1.00 | 20.83 |
| ATOM | 162 | CE2 | PHE | 19 | −4.582 | 23.259 | 63.095 | 1.00 | 20.74 |
| ATOM | 163 | CZ | PHE | 19 | −3.995 | 23.137 | 61.837 | 1.00 | 22.45 |
| ATOM | 164 | C | PHE | 19 | 0.075 | 24.985 | 64.678 | 1.00 | 20.64 |
| ATOM | 165 | O | PHE | 19 | −0.208 | 25.687 | 63.708 | 1.00 | 20.24 |
| ATOM | 166 | N | ALA | 20 | 1.298 | 24.509 | 64.894 | 1.00 | 17.95 |
| ATOM | 167 | CA | ALA | 20 | 2.409 | 24.801 | 63.994 | 1.00 | 18.49 |
| ATOM | 168 | CB | ALA | 20 | 3.671 | 25.051 | 64.808 | 1.00 | 16.95 |
| ATOM | 169 | C | ALA | 20 | 2.676 | 23.705 | 62.959 | 1.00 | 17.84 |
| ATOM | 170 | O | ALA | 20 | 2.563 | 22.515 | 63.253 | 1.00 | 19.20 |
| ATOM | 171 | N | THR | 21 | 3.035 | 24.126 | 61.750 | 1.00 | 19.33 |
| ATOM | 172 | CA | THR | 21 | 3.351 | 23.211 | 60.654 | 1.00 | 21.03 |
| ATOM | 173 | CB | THR | 21 | 2.235 | 23.215 | 59.595 | 1.00 | 22.40 |
| ATOM | 174 | OG1 | THR | 21 | 1.013 | 22.766 | 60.201 | 1.00 | 25.51 |
| ATOM | 175 | CG2 | THR | 21 | 2.583 | 22.298 | 58.449 | 1.00 | 28.47 |
| ATOM | 176 | C | THR | 21 | 4.667 | 23.668 | 60.022 | 1.00 | 18.87 |
| ATOM | 177 | O | THR | 21 | 5.028 | 24.846 | 60.095 | 1.00 | 19.63 |
| ATOM | 178 | N | ILE | 22 | 5.391 | 22.757 | 59.387 | 1.00 | 17.33 |
| ATOM | 179 | CA | ILE | 22 | 6.672 | 23.156 | 58.822 | 1.00 | 16.75 |
| ATOM | 180 | CB | ILE | 22 | 7.761 | 23.058 | 59.915 | 1.00 | 16.67 |
| ATOM | 181 | CG2 | ILE | 22 | 8.068 | 21.593 | 60.208 | 1.00 | 16.67 |
| ATOM | 182 | CG1 | ILE | 22 | 9.009 | 23.833 | 59.492 | 1.00 | 18.38 |
| ATOM | 183 | CD1 | ILE | 22 | 9.959 | 24.160 | 60.653 | 1.00 | 19.13 |
| ATOM | 184 | C | ILE | 22 | 7.068 | 22.314 | 57.617 | 1.00 | 16.22 |
| ATOM | 185 | O | ILE | 22 | 6.592 | 21.194 | 57.459 | 1.00 | 16.81 |
| ATOM | 186 | N | THR | 23 | 7.911 | 22.868 | 56.754 | 1.00 | 15.76 |
| ATOM | 187 | CA | THR | 23 | 8.357 | 22.119 | 55.586 | 1.00 | 19.36 |
| ATOM | 188 | CB | THR | 23 | 8.756 | 23.061 | 54.409 | 1.00 | 18.72 |
| ATOM | 189 | OG1 | THR | 23 | 10.010 | 23.697 | 54.692 | 1.00 | 23.35 |
| ATOM | 190 | CG2 | THR | 23 | 7.699 | 24.155 | 54.216 | 1.00 | 23.17 |
| ATOM | 191 | C | THR | 23 | 9.564 | 21.285 | 56.014 | 1.00 | 18.45 |
| ATOM | 192 | O | THR | 23 | 10.274 | 21.643 | 56.954 | 1.00 | 18.13 |
| ATOM | 193 | N | ALA | 24 | 9.772 | 20.155 | 55.343 | 1.00 | 17.71 |
| ATOM | 194 | CA | ALA | 24 | 10.897 | 19.276 | 55.633 | 1.00 | 16.52 |
| ATOM | 195 | CB | ALA | 24 | 10.575 | 18.345 | 56.796 | 1.00 | 16.73 |
| ATOM | 196 | C | ALA | 24 | 11.132 | 18.483 | 54.358 | 1.00 | 15.72 |
| ATOM | 197 | O | ALA | 24 | 10.181 | 18.183 | 53.634 | 1.00 | 13.61 |
| ATOM | 198 | N | TYR | 25 | 12.387 | 18.148 | 54.079 | 1.00 | 15.44 |
| ATOM | 199 | CA | TYR | 25 | 12.713 | 17.420 | 52.859 | 1.00 | 15.30 |
| ATOM | 200 | CB | TYR | 25 | 13.205 | 18.389 | 51.780 | 1.00 | 16.75 |
| ATOM | 201 | CG | TYR | 25 | 12.454 | 19.697 | 51.729 | 1.00 | 19.07 |
| ATOM | 202 | CD1 | TYR | 25 | 12.934 | 20.822 | 52.402 | 1.00 | 17.12 |
| ATOM | 203 | CE1 | TYR | 25 | 12.240 | 22.026 | 52.379 | 1.00 | 22.53 |
| ATOM | 204 | CD2 | TYR | 25 | 11.255 | 19.808 | 51.028 | 1.00 | 17.14 |
| ATOM | 205 | CE2 | TYR | 25 | 10.546 | 21.010 | 50.999 | 1.00 | 21.05 |
| ATOM | 206 | CZ | TYR | 25 | 11.044 | 22.114 | 51.678 | 1.00 | 22.44 |
| ATOM | 207 | OH | TYR | 25 | 10.347 | 23.300 | 51.669 | 1.00 | 25.31 |
| ATOM | 208 | C | TYR | 25 | 13.785 | 16.359 | 53.068 | 1.00 | 17.26 |
| ATOM | 209 | O | TYR | 25 | 14.327 | 15.837 | 52.094 | 1.00 | 16.21 |
| ATOM | 210 | N | ASP | 26 | 14.101 | 16.051 | 54.320 | 1.00 | 15.99 |
| ATOM | 211 | CA | ASP | 26 | 15.121 | 15.059 | 54.604 | 1.00 | 16.04 |
| ATOM | 212 | CB | ASP | 26 | 16.511 | 15.686 | 54.453 | 1.00 | 14.69 |
| ATOM | 213 | CG | ASP | 26 | 16.803 | 16.751 | 55.507 | 1.00 | 16.04 |
| ATOM | 214 | OD1 | ASP | 26 | 17.002 | 16.391 | 56.679 | 1.00 | 17.13 |
| ATOM | 215 | OD2 | ASP | 26 | 16.829 | 17.935 | 55.144 | 1.00 | 14.58 |
| ATOM | 216 | C | ASP | 26 | 14.967 | 14.426 | 55.981 | 1.00 | 16.01 |
| ATOM | 217 | O | ASP | 26 | 14.182 | 14.888 | 56.813 | 1.00 | 16.69 |
| ATOM | 218 | N | TYR | 27 | 15.718 | 13.353 | 56.214 | 1.00 | 13.50 |
| ATOM | 219 | CA | TYR | 27 | 15.660 | 12.625 | 57.474 | 1.00 | 15.85 |
| ATOM | 220 | CB | TYR | 27 | 16.591 | 11.412 | 57.408 | 1.00 | 16.76 |
| ATOM | 221 | CG | TYR | 27 | 16.777 | 10.693 | 58.727 | 1.00 | 17.84 |
| ATOM | 222 | CD1 | TYR | 27 | 15.871 | 9.723 | 59.150 | 1.00 | 17.06 |
| ATOM | 223 | CE1 | TYR | 27 | 16.053 | 9.046 | 60.353 | 1.00 | 18.09 |
| ATOM | 224 | CD2 | TYR | 27 | 17.873 | 10.975 | 59.546 | 1.00 | 19.28 |
| ATOM | 225 | CE2 | TYR | 27 | 18.065 | 10.311 | 60.748 | 1.00 | 20.21 |
| ATOM | 226 | CZ | TYR | 27 | 17.163 | 9.350 | 61.151 | 1.00 | 20.97 |
| ATOM | 227 | OH | TYR | 27 | 17.368 | 8.691 | 62.342 | 1.00 | 21.54 |
| ATOM | 228 | C | TYR | 27 | 16.056 | 13.482 | 58.671 | 1.00 | 15.76 |
| ATOM | 229 | O | TYR | 27 | 15.338 | 13.544 | 59.670 | 1.00 | 16.46 |
| ATOM | 230 | N | SER | 28 | 17.216 | 14.121 | 58.560 | 1.00 | 16.97 |
| ATOM | 231 | CA | SER | 28 | 17.763 | 14.943 | 59.630 | 1.00 | 17.00 |
| ATOM | 232 | CB | SER | 28 | 19.034 | 15.643 | 59.146 | 1.00 | 19.12 |
| ATOM | 233 | OG | SER | 28 | 20.029 | 14.671 | 58.842 | 1.00 | 22.00 |
| ATOM | 234 | C | SER | 28 | 16.798 | 15.957 | 60.222 | 1.00 | 17.23 |
| ATOM | 235 | O | SER | 28 | 16.485 | 15.905 | 61.422 | 1.00 | 16.02 |
| ATOM | 236 | N | PHE | 29 | 16.307 | 16.881 | 59.408 | 1.00 | 15.80 |
| ATOM | 237 | CA | PHE | 29 | 15.382 | 17.864 | 59.965 | 1.00 | 16.28 |
| ATOM | 238 | CB | PHE | 29 | 15.181 | 19.025 | 59.000 | 1.00 | 14.20 |
| ATOM | 239 | CG | PHE | 29 | 16.321 | 19.988 | 59.001 | 1.00 | 15.71 |
| ATOM | 240 | CD1 | PHE | 29 | 17.354 | 19.871 | 58.075 | 1.00 | 14.27 |
| ATOM | 241 | CD2 | PHE | 29 | 16.371 | 21.008 | 59.946 | 1.00 | 13.32 |
| ATOM | 242 | CE1 | PHE | 29 | 18.423 | 20.764 | 58.080 | 1.00 | 15.74 |
| ATOM | 243 | CE2 | PHE | 29 | 17.430 | 21.904 | 59.967 | 1.00 | 17.20 |
| ATOM | 244 | CZ | PHE | 29 | 18.463 | 21.787 | 59.031 | 1.00 | 14.83 |
| ATOM | 245 | C | PHE | 29 | 14.044 | 17.284 | 60.383 | 1.00 | 15.83 |
| ATOM | 246 | O | PHE | 29 | 13.481 | 17.696 | 61.398 | 1.00 | 16.89 |
| ATOM | 247 | N | ALA | 30 | 13.532 | 16.326 | 59.618 | 1.00 | 16.16 |
| ATOM | 248 | CA | ALA | 30 | 12.256 | 15.718 | 59.962 | 1.00 | 17.31 |
| ATOM | 249 | CB | ALA | 30 | 11.887 | 14.649 | 58.925 | 1.00 | 16.54 |
| ATOM | 250 | C | ALA | 30 | 12.343 | 15.094 | 61.357 | 1.00 | 17.30 |
| ATOM | 251 | O | ALA | 30 | 11.404 | 15.171 | 62.155 | 1.00 | 16.42 |
| ATOM | 252 | N | LYS | 31 | 13.481 | 14.467 | 61.634 | 1.00 | 16.26 |
| ATOM | 253 | CA | LYS | 31 | 13.731 | 13.815 | 62.918 | 1.00 | 17.07 |
| ATOM | 254 | CB | LYS | 31 | 15.062 | 13.063 | 62.852 | 1.00 | 18.24 |
| ATOM | 255 | CG | LYS | 31 | 15.491 | 12.386 | 64.146 | 1.00 | 24.20 |
| ATOM | 256 | CD | LYS | 31 | 14.608 | 11.203 | 64.469 | 1.00 | 27.80 |
| ATOM | 257 | CE | LYS | 31 | 15.306 | 10.248 | 65.425 | 1.00 | 30.36 |
| ATOM | 258 | NZ | LYS | 31 | 15.724 | 10.913 | 66.697 | 1.00 | 32.82 |
| ATOM | 259 | C | LYS | 31 | 13.788 | 14.833 | 64.057 | 1.00 | 17.16 |
| ATOM | 260 | O | LYS | 31 | 13.250 | 14.608 | 65.147 | 1.00 | 18.34 |
| ATOM | 261 | N | LEU | 32 | 14.468 | 15.941 | 63.790 | 1.00 | 18.82 |
| ATOM | 262 | CA | LEU | 32 | 14.631 | 17.019 | 64.756 | 1.00 | 18.15 |
| ATOM | 263 | CB | LEU | 32 | 15.549 | 18.097 | 64.171 | 1.00 | 17.24 |
| ATOM | 264 | CG | LEU | 32 | 16.070 | 19.200 | 65.113 | 1.00 | 18.88 |
| ATOM | 265 | CD1 | LEU | 32 | 17.356 | 19.769 | 64.556 | 1.00 | 17.20 |
| ATOM | 266 | CD2 | LEU | 32 | 15.008 | 20.292 | 65.280 | 1.00 | 18.78 |
| ATOM | 267 | C | LEU | 32 | 13.272 | 17.620 | 65.103 | 1.00 | 18.02 |
| ATOM | 268 | O | LEU | 32 | 12.963 | 17.847 | 66.272 | 1.00 | 17.18 |
| ATOM | 269 | N | PHE | 33 | 12.462 | 17.885 | 64.083 | 1.00 | 17.34 |
| ATOM | 270 | CA | PHE | 33 | 11.144 | 18.473 | 64.316 | 1.00 | 18.21 |
| ATOM | 271 | CB | PHE | 33 | 10.451 | 18.832 | 62.995 | 1.00 | 15.31 |
| ATOM | 272 | CG | PHE | 33 | 11.255 | 19.734 | 62.092 | 1.00 | 14.93 |
| ATOM | 273 | CD1 | PHE | 33 | 12.133 | 20.689 | 62.610 | 1.00 | 14.65 |
| ATOM | 274 | CD2 | PHE | 33 | 11.093 | 19.657 | 60.716 | 1.00 | 13.00 |
| ATOM | 275 | CE1 | PHE | 33 | 12.832 | 21.550 | 61.764 | 1.00 | 13.96 |
| ATOM | 276 | CE2 | PHE | 33 | 11.783 | 20.510 | 59.861 | 1.00 | 9.75 |
| ATOM | 277 | CZ | PHE | 33 | 12.657 | 21.461 | 60.389 | 1.00 | 14.73 |
| ATOM | 278 | C | PHE | 33 | 10.255 | 17.503 | 65.091 | 1.00 | 18.11 |
| ATOM | 279 | O | PHE | 33 | 9.582 | 17.892 | 66.048 | 1.00 | 17.51 |
| ATOM | 280 | N | ALA | 34 | 10.246 | 16.241 | 64.666 | 1.00 | 18.42 |
| ATOM | 281 | CA | ALA | 34 | 9.433 | 15.231 | 65.330 | 1.00 | 19.46 |
| ATOM | 282 | CB | ALA | 34 | 9.573 | 13.878 | 64.623 | 1.00 | 20.93 |
| ATOM | 283 | C | ALA | 34 | 9.828 | 15.098 | 66.799 | 1.00 | 20.01 |
| ATOM | 284 | O | ALA | 34 | 8.970 | 15.000 | 67.673 | 1.00 | 19.41 |
| ATOM | 285 | N | ASP | 35 | 11.125 | 15.101 | 67.074 | 1.00 | 22.13 |
| ATOM | 286 | CA | ASP | 35 | 11.574 | 14.972 | 68.449 | 1.00 | 24.53 |
| ATOM | 287 | CB | ASP | 35 | 13.086 | 14.788 | 68.503 | 1.00 | 25.01 |
| ATOM | 288 | CG | ASP | 35 | 13.522 | 13.424 | 67.989 | 1.00 | 27.07 |
| ATOM | 289 | OD1 | ASP | 35 | 12.665 | 12.519 | 67.898 | 1.00 | 28.23 |
| ATOM | 290 | OD2 | ASP | 35 | 14.720 | 13.261 | 67.694 | 1.00 | 28.18 |
| ATOM | 291 | C | ASP | 35 | 11.156 | 16.151 | 69.324 | 1.00 | 23.50 |
| ATOM | 292 | O | ASP | 35 | 11.086 | 16.024 | 70.545 | 1.00 | 24.71 |
| ATOM | 293 | N | GLU | 36 | 10.872 | 17.294 | 68.706 | 1.00 | 24.53 |
| ATOM | 294 | CA | GLU | 36 | 10.455 | 18.469 | 69.464 | 1.00 | 23.98 |
| ATOM | 295 | CB | GLU | 36 | 11.029 | 19.743 | 68.841 | 1.00 | 25.45 |
| ATOM | 296 | CG | GLU | 36 | 12.535 | 19.876 | 68.963 | 1.00 | 25.52 |
| ATOM | 297 | CD | GLU | 36 | 13.005 | 19.750 | 70.399 | 1.00 | 26.66 |
| ATOM | 298 | OE1 | GLU | 36 | 12.385 | 20.375 | 71.286 | 1.00 | 23.68 |
| ATOM | 299 | OE2 | GLU | 36 | 13.993 | 19.032 | 70.637 | 1.00 | 27.91 |
| ATOM | 300 | C | GLU | 36 | 8.937 | 18.588 | 69.553 | 1.00 | 23.45 |
| ATOM | 301 | O | GLU | 36 | 8.421 | 19.502 | 70.192 | 1.00 | 23.61 |
| ATOM | 302 | N | GLY | 37 | 8.221 | 17.672 | 68.908 | 1.00 | 22.87 |
| ATOM | 303 | CA | GLY | 37 | 6.765 | 17.721 | 68.955 | 1.00 | 22.54 |
| ATOM | 304 | C | GLY | 37 | 6.096 | 18.447 | 67.796 | 1.00 | 19.79 |
| ATOM | 305 | O | GLY | 37 | 4.902 | 18.741 | 67.845 | 1.00 | 21.78 |
| ATOM | 306 | N | LEU | 38 | 6.873 | 18.755 | 66.765 | 1.00 | 19.44 |
| ATOM | 307 | CA | LEU | 38 | 6.365 | 19.416 | 65.569 | 1.00 | 19.71 |
| ATOM | 308 | CB | LEU | 38 | 7.459 | 20.281 | 64.952 | 1.00 | 20.11 |
| ATOM | 309 | CG | LEU | 38 | 7.131 | 21.691 | 64.458 | 1.00 | 22.03 |
| ATOM | 310 | CD1 | LEU | 38 | 8.352 | 22.238 | 63.739 | 1.00 | 20.65 |
| ATOM | 311 | CD2 | LEU | 38 | 5.918 | 21.714 | 63.548 | 1.00 | 20.80 |
| ATOM | 312 | C | LEU | 38 | 6.057 | 18.222 | 64.664 | 1.00 | 20.90 |
| ATOM | 313 | O | LEU | 38 | 6.938 | 17.741 | 63.939 | 1.00 | 18.42 |
| ATOM | 314 | N | ASN | 39 | 4.807 | 17.762 | 64.720 | 1.00 | 20.83 |
| ATOM | 315 | CA | ASN | 39 | 4.355 | 16.573 | 63.999 | 1.00 | 21.62 |
| ATOM | 316 | CB | ASN | 39 | 3.489 | 15.709 | 64.924 | 1.00 | 24.31 |
| ATOM | 317 | CG | ASN | 39 | 4.128 | 15.480 | 66.281 | 1.00 | 28.12 |
| ATOM | 318 | OD1 | ASN | 39 | 5.334 | 15.252 | 66.377 | 1.00 | 28.10 |
| ATOM | 319 | ND2 | ASN | 39 | 3.321 | 15.526 | 67.338 | 1.00 | 29.42 |
| ATOM | 320 | C | ASN | 39 | 3.593 | 16.766 | 62.696 | 1.00 | 19.52 |
| ATOM | 321 | O | ASN | 39 | 2.955 | 15.832 | 62.221 | 1.00 | 19.99 |
| ATOM | 322 | N | VAL | 40 | 3.648 | 17.961 | 62.123 | 1.00 | 19.39 |
| ATOM | 323 | CA | VAL | 40 | 2.960 | 18.221 | 60.869 | 1.00 | 18.52 |
| ATOM | 324 | CB | VAL | 40 | 1.799 | 19.213 | 61.056 | 1.00 | 19.51 |
| ATOM | 325 | CG1 | VAL | 40 | 1.113 | 19.470 | 59.726 | 1.00 | 18.68 |
| ATOM | 326 | CG2 | VAL | 40 | 0.801 | 18.659 | 62.066 | 1.00 | 18.86 |
| ATOM | 327 | C | VAL | 40 | 3.967 | 18.785 | 59.888 | 1.00 | 18.22 |
| ATOM | 328 | O | VAL | 40 | 4.450 | 19.910 | 60.040 | 1.00 | 14.68 |
| ATOM | 329 | N | MET | 41 | 4.280 | 17.990 | 58.869 | 1.00 | 16.95 |
| ATOM | 330 | CA | MET | 41 | 5.271 | 18.398 | 57.892 | 1.00 | 16.29 |
| ATOM | 331 | CB | MET | 41 | 6.535 | 17.556 | 58.082 | 1.00 | 16.97 |
| ATOM | 332 | CG | MET | 41 | 7.255 | 17.864 | 59.392 | 1.00 | 20.16 |
| ATOM | 333 | SD | MET | 41 | 8.564 | 16.706 | 59.719 | 1.00 | 19.46 |
| ATOM | 334 | CE | MET | 41 | 7.955 | 15.899 | 61.206 | 1.00 | 21.43 |
| ATOM | 335 | C | MET | 41 | 4.804 | 18.342 | 56.452 | 1.00 | 16.56 |
| ATOM | 336 | O | MET | 41 | 4.046 | 17.464 | 56.062 | 1.00 | 18.81 |
| ATOM | 337 | N | LEU | 42 | 5.276 | 19.300 | 55.665 | 1.00 | 18.90 |
| ATOM | 338 | CA | LEU | 42 | 4.907 | 19.383 | 54.265 | 1.00 | 20.11 |
| ATOM | 339 | CB | LEU | 42 | 4.178 | 20.707 | 54.016 | 1.00 | 22.93 |
| ATOM | 340 | CG | LEU | 42 | 3.677 | 21.143 | 52.630 | 1.00 | 26.24 |
| ATOM | 341 | CD1 | LEU | 42 | 4.777 | 21.879 | 51.907 | 1.00 | 29.83 |
| ATOM | 342 | CD2 | LEU | 42 | 3.169 | 19.955 | 51.818 | 1.00 | 24.10 |
| ATOM | 343 | C | LEU | 42 | 6.120 | 19.258 | 53.344 | 1.00 | 19.47 |
| ATOM | 344 | O | LEU | 42 | 7.106 | 19.978 | 53.488 | 1.00 | 17.61 |
| ATOM | 345 | N | VAL | 43 | 6.045 | 18.313 | 52.414 | 1.00 | 19.14 |
| ATOM | 346 | CA | VAL | 43 | 7.102 | 18.116 | 51.429 | 1.00 | 18.25 |
| ATOM | 347 | CB | VAL | 43 | 7.332 | 16.624 | 51.126 | 1.00 | 19.90 |
| ATOM | 348 | CG1 | VAL | 43 | 8.397 | 16.461 | 50.041 | 1.00 | 19.97 |
| ATOM | 349 | CG2 | VAL | 43 | 7.752 | 15.895 | 52.395 | 1.00 | 19.98 |
| ATOM | 350 | C | VAL | 43 | 6.517 | 18.806 | 50.208 | 1.00 | 18.93 |
| ATOM | 351 | O | VAL | 43 | 5.815 | 18.187 | 49.408 | 1.00 | 17.33 |
| ATOM | 352 | N | GLY | 44 | 6.786 | 20.102 | 50.089 | 1.00 | 17.93 |
| ATOM | 353 | CA | GLY | 44 | 6.248 | 20.865 | 48.980 | 1.00 | 20.04 |
| ATOM | 354 | C | GLY | 44 | 7.226 | 21.095 | 47.854 | 1.00 | 18.04 |
| ATOM | 355 | O | GLY | 44 | 8.430 | 20.872 | 48.008 | 1.00 | 17.40 |
| ATOM | 356 | N | ASP | 45 | 6.715 | 21.557 | 46.718 | 1.00 | 18.65 |
| ATOM | 357 | CA | ASP | 45 | 7.583 | 21.796 | 45.575 | 1.00 | 20.74 |
| ATOM | 358 | CB | ASP | 45 | 6.764 | 21.936 | 44.279 | 1.00 | 21.58 |
| ATOM | 359 | CG | ASP | 45 | 5.684 | 22.997 | 44.372 | 1.00 | 23.34 |
| ATOM | 360 | OD1 | ASP | 45 | 5.672 | 23.751 | 45.359 | 1.00 | 22.56 |
| ATOM | 361 | OD2 | ASP | 45 | 4.858 | 23.074 | 43.442 | 1.00 | 21.88 |
| ATOM | 362 | C | ASP | 45 | 8.483 | 23.007 | 45.776 | 1.00 | 19.43 |
| ATOM | 363 | O | ASP | 45 | 9.243 | 23.374 | 44.883 | 1.00 | 19.95 |
| ATOM | 364 | N | SER | 46 | 8.408 | 23.635 | 46.949 | 1.00 | 20.64 |
| ATOM | 365 | CA | SER | 46 | 9.285 | 24.772 | 47.211 | 1.00 | 19.31 |
| ATOM | 366 | CB | SER | 46 | 8.944 | 25.429 | 48.551 | 1.00 | 19.58 |
| ATOM | 367 | OG | SER | 46 | 9.146 | 24.533 | 49.628 | 1.00 | 20.11 |
| ATOM | 368 | C | SER | 46 | 10.705 | 24.206 | 47.243 | 1.00 | 19.27 |
| ATOM | 369 | O | SER | 46 | 11.686 | 24.945 | 47.155 | 1.00 | 20.36 |
| ATOM | 370 | N | LEU | 47 | 10.810 | 22.884 | 47.365 | 1.00 | 18.59 |
| ATOM | 371 | CA | LEU | 47 | 12.119 | 22.237 | 47.384 | 1.00 | 19.25 |
| ATOM | 372 | CB | LEU | 47 | 11.970 | 20.737 | 47.661 | 1.00 | 18.00 |
| ATOM | 373 | CG | LEU | 47 | 11.308 | 19.853 | 46.597 | 1.00 | 17.40 |
| ATOM | 374 | CD1 | LEU | 47 | 12.309 | 19.517 | 45.489 | 1.00 | 15.15 |
| ATOM | 375 | CD2 | LEU | 47 | 10.822 | 18.572 | 47.255 | 1.00 | 16.79 |
| ATOM | 376 | C | LEU | 47 | 12.853 | 22.467 | 46.063 | 1.00 | 17.82 |
| ATOM | 377 | O | LEU | 47 | 14.083 | 22.399 | 46.004 | 1.00 | 17.99 |
| ATOM | 378 | N | GLY | 48 | 12.100 | 22.742 | 45.001 | 1.00 | 17.81 |
| ATOM | 379 | CA | GLY | 48 | 12.720 | 22.997 | 43.708 | 1.00 | 16.18 |
| ATOM | 380 | C | GLY | 48 | 13.659 | 24.186 | 43.784 | 1.00 | 18.66 |
| ATOM | 381 | O | GLY | 48 | 14.644 | 24.271 | 43.050 | 1.00 | 17.89 |
| ATOM | 382 | N | MET | 49 | 13.356 | 25.108 | 44.691 | 1.00 | 19.84 |
| ATOM | 383 | CA | MET | 49 | 14.172 | 26.303 | 44.865 | 1.00 | 20.69 |
| ATOM | 384 | CB | MET | 49 | 13.263 | 27.515 | 45.100 | 1.00 | 23.46 |
| ATOM | 385 | CG | MET | 49 | 12.312 | 27.789 | 43.940 | 1.00 | 25.97 |
| ATOM | 386 | SD | MET | 49 | 11.099 | 29.069 | 44.266 | 1.00 | 31.34 |
| ATOM | 387 | CE | MET | 49 | 12.037 | 30.520 | 43.900 | 1.00 | 32.33 |
| ATOM | 388 | C | MET | 49 | 15.162 | 26.153 | 46.022 | 1.00 | 20.49 |
| ATOM | 389 | O | MET | 49 | 16.370 | 26.292 | 45.837 | 1.00 | 22.08 |
| ATOM | 390 | N | THR | 50 | 14.649 | 25.846 | 47.208 | 1.00 | 21.41 |
| ATOM | 391 | CA | THR | 50 | 15.492 | 25.702 | 48.387 | 1.00 | 21.56 |
| ATOM | 392 | CB | THR | 50 | 14.636 | 25.608 | 49.655 | 1.00 | 25.24 |
| ATOM | 393 | OG1 | THR | 50 | 15.493 | 25.523 | 50.801 | 1.00 | 31.31 |
| ATOM | 394 | CG2 | THR | 50 | 13.750 | 24.381 | 49.600 | 1.00 | 23.80 |
| ATOM | 395 | C | THR | 50 | 16.432 | 24.498 | 48.362 | 1.00 | 21.30 |
| ATOM | 396 | O | THR | 50 | 17.551 | 24.565 | 48.864 | 1.00 | 21.10 |
| ATOM | 397 | N | VAL | 51 | 15.975 | 23.391 | 47.787 | 1.00 | 18.20 |
| ATOM | 398 | CA | VAL | 51 | 16.812 | 22.200 | 47.719 | 1.00 | 19.81 |
| ATOM | 399 | CB | VAL | 51 | 15.997 | 20.918 | 47.983 | 1.00 | 18.02 |
| ATOM | 400 | CG1 | VAL | 51 | 16.909 | 19.695 | 47.900 | 1.00 | 23.01 |
| ATOM | 401 | CG2 | VAL | 51 | 15.355 | 20.983 | 49.365 | 1.00 | 21.92 |
| ATOM | 402 | C | VAL | 51 | 17.536 | 22.038 | 46.384 | 1.00 | 18.45 |
| ATOM | 403 | O | VAL | 51 | 18.755 | 21.867 | 46.351 | 1.00 | 16.95 |
| ATOM | 404 | N | GLN | 52 | 16.788 | 22.087 | 45.286 | 1.00 | 17.92 |
| ATOM | 405 | CA | GLN | 52 | 17.381 | 21.907 | 43.963 | 1.00 | 18.16 |
| ATOM | 406 | CB | GLN | 52 | 16.312 | 21.427 | 42.976 | 1.00 | 17.32 |
| ATOM | 407 | CG | GLN | 52 | 15.529 | 20.227 | 43.482 | 1.00 | 16.59 |
| ATOM | 408 | CD | GLN | 52 | 14.477 | 19.771 | 42.504 | 1.00 | 15.57 |
| ATOM | 409 | OE1 | GLN | 52 | 14.011 | 20.552 | 41.673 | 1.00 | 16.27 |
| ATOM | 410 | NE2 | GLN | 52 | 14.084 | 18.504 | 42.600 | 1.00 | 14.83 |
| ATOM | 411 | C | GLN | 52 | 18.076 | 23.150 | 43.411 | 1.00 | 18.00 |
| ATOM | 412 | O | GLN | 52 | 19.003 | 23.052 | 42.606 | 1.00 | 18.07 |
| ATOM | 413 | N | GLY | 53 | 17.624 | 24.324 | 43.831 | 1.00 | 19.93 |
| ATOM | 414 | CA | GLY | 53 | 18.248 | 25.549 | 43.361 | 1.00 | 20.83 |
| ATOM | 415 | C | GLY | 53 | 17.685 | 26.146 | 42.085 | 1.00 | 22.32 |
| ATOM | 416 | O | GLY | 53 | 18.387 | 26.877 | 41.387 | 1.00 | 24.49 |
| ATOM | 417 | N | HIS | 54 | 16.429 | 25.849 | 41.771 | 1.00 | 22.93 |
| ATOM | 418 | CA | HIS | 54 | 15.803 | 26.397 | 40.575 | 1.00 | 24.27 |
| ATOM | 419 | CB | HIS | 54 | 14.725 | 25.438 | 40.049 | 1.00 | 23.64 |
| ATOM | 420 | CG | HIS | 54 | 15.264 | 24.127 | 39.568 | 1.00 | 25.03 |
| ATOM | 421 | CD2 | HIS | 54 | 15.030 | 22.860 | 39.984 | 1.00 | 25.14 |
| ATOM | 422 | ND1 | HIS | 54 | 16.175 | 24.029 | 38.538 | 1.00 | 25.82 |
| ATOM | 423 | CE1 | HIS | 54 | 16.481 | 22.759 | 38.343 | 1.00 | 26.94 |
| ATOM | 424 | NE2 | HIS | 54 | 15.800 | 22.029 | 39.208 | 1.00 | 26.01 |
| ATOM | 425 | C | HIS | 54 | 15.176 | 27.748 | 40.914 | 1.00 | 25.18 |
| ATOM | 426 | O | HIS | 54 | 14.947 | 28.058 | 42.086 | 1.00 | 24.14 |
| ATOM | 427 | N | ASP | 55 | 14.898 | 28.545 | 39.884 | 1.00 | 25.82 |
| ATOM | 428 | CA | ASP | 55 | 14.302 | 29.869 | 40.062 | 1.00 | 27.59 |
| ATOM | 429 | CB | ASP | 55 | 14.550 | 30.729 | 38.813 | 1.00 | 30.40 |
| ATOM | 430 | CG | ASP | 55 | 13.786 | 30.232 | 37.600 | 1.00 | 33.05 |
| ATOM | 431 | OD1 | ASP | 55 | 12.535 | 30.266 | 37.622 | 1.00 | 36.66 |
| ATOM | 432 | OD2 | ASP | 55 | 14.428 | 29.804 | 36.618 | 1.00 | 37.40 |
| ATOM | 433 | C | ASP | 55 | 12.801 | 29.778 | 40.333 | 1.00 | 26.78 |
| ATOM | 434 | O | ASP | 55 | 12.174 | 30.755 | 40.737 | 1.00 | 27.57 |
| ATOM | 435 | N | SER | 56 | 12.228 | 28.600 | 40.099 | 1.00 | 25.17 |
| ATOM | 436 | CA | SER | 56 | 10.802 | 28.386 | 40.322 | 1.00 | 21.97 |
| ATOM | 437 | CB | SER | 56 | 10.008 | 28.635 | 39.036 | 1.00 | 21.35 |
| ATOM | 438 | OG | SER | 56 | 10.232 | 27.608 | 38.084 | 1.00 | 21.31 |
| ATOM | 439 | C | SER | 56 | 10.582 | 26.956 | 40.795 | 1.00 | 20.02 |
| ATOM | 440 | O | SER | 56 | 11.529 | 26.177 | 40.903 | 1.00 | 21.16 |
| ATOM | 441 | N | THR | 57 | 9.334 | 26.615 | 41.084 | 1.00 | 19.01 |
| ATOM | 442 | CA | THR | 57 | 9.007 | 25.272 | 41.543 | 1.00 | 19.21 |
| ATOM | 443 | CB | THR | 57 | 7.869 | 25.305 | 42.579 | 1.00 | 19.53 |
| ATOM | 444 | OG1 | THR | 57 | 6.686 | 25.840 | 41.972 | 1.00 | 20.97 |
| ATOM | 445 | CG2 | THR | 57 | 8.249 | 26.179 | 43.772 | 1.00 | 19.07 |
| ATOM | 446 | C | THR | 57 | 8.560 | 24.359 | 40.396 | 1.00 | 18.44 |
| ATOM | 447 | O | THR | 57 | 8.422 | 23.153 | 40.587 | 1.00 | 18.34 |
| ATOM | 448 | N | LEU | 58 | 8.341 | 24.930 | 39.212 | 1.00 | 20.59 |
| ATOM | 449 | CA | LEU | 58 | 7.868 | 24.149 | 38.062 | 1.00 | 20.34 |
| ATOM | 450 | CB | LEU | 58 | 7.720 | 25.029 | 36.816 | 1.00 | 20.92 |
| ATOM | 451 | CG | LEU | 58 | 6.542 | 26.005 | 36.785 | 1.00 | 23.24 |
| ATOM | 452 | CD1 | LEU | 58 | 6.926 | 27.242 | 37.578 | 1.00 | 25.30 |
| ATOM | 453 | CD2 | LEU | 58 | 6.195 | 26.393 | 35.349 | 1.00 | 23.02 |
| ATOM | 454 | C | LEU | 58 | 8.687 | 22.913 | 37.696 | 1.00 | 21.07 |
| ATOM | 455 | O | LEU | 58 | 8.120 | 21.868 | 37.366 | 1.00 | 19.23 |
| ATOM | 456 | N | PRO | 59 | 10.025 | 23.015 | 37.731 | 1.00 | 20.10 |
| ATOM | 457 | CD | PRO | 59 | 10.845 | 24.213 | 37.965 | 1.00 | 23.05 |
| ATOM | 458 | CA | PRO | 59 | 10.862 | 21.860 | 37.392 | 1.00 | 21.47 |
| ATOM | 459 | CB | PRO | 59 | 12.286 | 22.433 | 37.427 | 1.00 | 21.53 |
| ATOM | 460 | CG | PRO | 59 | 12.167 | 23.617 | 38.335 | 1.00 | 25.99 |
| ATOM | 461 | C | PRO | 59 | 10.678 | 20.653 | 38.307 | 1.00 | 18.05 |
| ATOM | 462 | O | PRO | 59 | 11.041 | 19.534 | 37.946 | 1.00 | 18.36 |
| ATOM | 463 | N | VAL | 60 | 10.100 | 20.865 | 39.486 | 1.00 | 17.05 |
| ATOM | 464 | CA | VAL | 60 | 9.882 | 19.760 | 40.423 | 1.00 | 16.87 |
| ATOM | 465 | CB | VAL | 60 | 9.330 | 20.273 | 41.785 | 1.00 | 15.81 |
| ATOM | 466 | CG1 | VAL | 60 | 9.046 | 19.099 | 42.709 | 1.00 | 15.77 |
| ATOM | 467 | CG2 | VAL | 60 | 10.336 | 21.201 | 42.438 | 1.00 | 15.49 |
| ATOM | 468 | C | VAL | 60 | 8.894 | 18.740 | 39.852 | 1.00 | 17.60 |
| ATOM | 469 | O | VAL | 60 | 7.805 | 19.099 | 39.406 | 1.00 | 16.90 |
| ATOM | 470 | N | THR | 61 | 9.267 | 17.465 | 39.876 | 1.00 | 18.90 |
| ATOM | 471 | CA | THR | 61 | 8.389 | 16.420 | 39.352 | 1.00 | 20.57 |
| ATOM | 472 | CB | THR | 61 | 9.124 | 15.590 | 38.252 | 1.00 | 24.68 |
| ATOM | 473 | OG1 | THR | 61 | 9.451 | 16.440 | 37.146 | 1.00 | 30.75 |
| ATOM | 474 | CG2 | THR | 61 | 8.261 | 14.425 | 37.760 | 1.00 | 30.08 |
| ATOM | 475 | C | THR | 61 | 7.906 | 15.507 | 40.487 | 1.00 | 17.77 |
| ATOM | 476 | O | THR | 61 | 8.408 | 15.581 | 41.606 | 1.00 | 16.02 |
| ATOM | 477 | N | VAL | 62 | 6.919 | 14.662 | 40.196 | 1.00 | 15.87 |
| ATOM | 478 | CA | VAL | 62 | 6.360 | 13.734 | 41.177 | 1.00 | 15.01 |
| ATOM | 479 | CB | VAL | 62 | 5.269 | 12.834 | 40.532 | 1.00 | 13.13 |
| ATOM | 480 | CG1 | VAL | 62 | 4.831 | 11.742 | 41.512 | 1.00 | 13.60 |
| ATOM | 481 | CG2 | VAL | 62 | 4.070 | 13.696 | 40.116 | 1.00 | 13.51 |
| ATOM | 482 | C | VAL | 62 | 7.428 | 12.837 | 41.784 | 1.00 | 15.22 |
| ATOM | 483 | O | VAL | 62 | 7.390 | 12.529 | 42.978 | 1.00 | 16.66 |
| ATOM | 484 | N | ALA | 63 | 8.383 | 12.412 | 40.965 | 1.00 | 15.74 |
| ATOM | 485 | CA | ALA | 63 | 9.445 | 11.551 | 41.467 | 1.00 | 14.86 |
| ATOM | 486 | CB | ALA | 63 | 10.383 | 11.137 | 40.319 | 1.00 | 16.20 |
| ATOM | 487 | C | ALA | 63 | 10.230 | 12.264 | 42.562 | 1.00 | 13.05 |
| ATOM | 488 | O | ALA | 63 | 10.579 | 11.655 | 43.573 | 1.00 | 13.87 |
| ATOM | 489 | N | ASP | 64 | 10.505 | 13.551 | 42.363 | 1.00 | 14.03 |
| ATOM | 490 | CA | ASP | 64 | 11.258 | 14.319 | 43.357 | 1.00 | 14.19 |
| ATOM | 491 | CB | ASP | 64 | 11.507 | 15.762 | 42.890 | 1.00 | 13.98 |
| ATOM | 492 | CG | ASP | 64 | 12.309 | 15.849 | 41.605 | 1.00 | 14.58 |
| ATOM | 493 | OD1 | ASP | 64 | 13.170 | 14.975 | 41.351 | 1.00 | 15.44 |
| ATOM | 494 | OD2 | ASP | 64 | 12.093 | 16.829 | 40.846 | 1.00 | 17.47 |
| ATOM | 495 | C | ASP | 64 | 10.492 | 14.355 | 44.679 | 1.00 | 14.42 |
| ATOM | 496 | O | ASP | 64 | 11.072 | 14.125 | 45.740 | 1.00 | 12.00 |
| ATOM | 497 | N | ILE | 65 | 9.194 | 14.649 | 44.618 | 1.00 | 13.48 |
| ATOM | 498 | CA | ILE | 65 | 8.374 | 14.705 | 45.827 | 1.00 | 13.80 |
| ATOM | 499 | CB | ILE | 65 | 6.899 | 15.082 | 45.504 | 1.00 | 11.05 |
| ATOM | 500 | CG2 | ILE | 65 | 6.042 | 14.958 | 46.773 | 1.00 | 13.85 |
| ATOM | 501 | CG1 | ILE | 65 | 6.822 | 16.488 | 44.929 | 1.00 | 14.20 |
| ATOM | 502 | CD1 | ILE | 65 | 7.176 | 17.602 | 45.914 | 1.00 | 13.32 |
| ATOM | 503 | C | ILE | 65 | 8.382 | 13.359 | 46.551 | 1.00 | 12.47 |
| ATOM | 504 | O | ILE | 65 | 8.502 | 13.294 | 47.769 | 1.00 | 13.03 |
| ATOM | 505 | N | ALA | 66 | 8.252 | 12.279 | 45.786 | 1.00 | 12.41 |
| ATOM | 506 | CA | ALA | 66 | 8.217 | 10.933 | 46.356 | 1.00 | 9.78 |
| ATOM | 507 | CB | ALA | 66 | 7.938 | 9.913 | 45.252 | 1.00 | 9.59 |
| ATOM | 508 | C | ALA | 66 | 9.518 | 10.582 | 47.077 | 1.00 | 11.07 |
| ATOM | 509 | O | ALA | 66 | 9.529 | 9.899 | 48.103 | 1.00 | 11.14 |
| ATOM | 510 | N | TYR | 67 | 10.619 | 11.023 | 46.492 | 1.00 | 11.42 |
| ATOM | 511 | CA | TYR | 67 | 11.944 | 10.790 | 47.048 | 1.00 | 12.07 |
| ATOM | 512 | CB | TYR | 67 | 12.977 | 11.335 | 46.061 | 1.00 | 11.65 |
| ATOM | 513 | CG | TYR | 67 | 14.394 | 11.327 | 46.566 | 1.00 | 13.46 |
| ATOM | 514 | CD1 | TYR | 67 | 15.120 | 10.146 | 46.641 | 1.00 | 13.65 |
| ATOM | 515 | CE1 | TYR | 67 | 16.441 | 10.144 | 47.081 | 1.00 | 15.17 |
| ATOM | 516 | CD2 | TYR | 67 | 15.018 | 12.515 | 46.949 | 1.00 | 16.34 |
| ATOM | 517 | CE2 | TYR | 67 | 16.333 | 12.529 | 47.389 | 1.00 | 13.50 |
| ATOM | 518 | CZ | TYR | 67 | 17.039 | 11.340 | 47.451 | 1.00 | 16.29 |
| ATOM | 519 | OH | TYR | 67 | 18.351 | 11.348 | 47.874 | 1.00 | 16.32 |
| ATOM | 520 | C | TYR | 67 | 12.082 | 11.487 | 48.414 | 1.00 | 12.63 |
| ATOM | 521 | O | TYR | 67 | 12.501 | 10.878 | 49.406 | 1.00 | 12.25 |
| ATOM | 522 | N | HIS | 68 | 11.713 | 12.765 | 48.455 | 1.00 | 14.09 |
| ATOM | 523 | CA | HIS | 68 | 11.814 | 13.548 | 49.688 | 1.00 | 13.34 |
| ATOM | 524 | CB | HIS | 68 | 11.723 | 15.039 | 49.358 | 1.00 | 12.95 |
| ATOM | 525 | CG | HIS | 68 | 12.930 | 15.561 | 48.644 | 1.00 | 13.09 |
| ATOM | 526 | CD2 | HIS | 68 | 13.146 | 15.833 | 47.335 | 1.00 | 13.59 |
| ATOM | 527 | ND1 | HIS | 68 | 14.128 | 15.794 | 49.285 | 1.00 | 11.39 |
| ATOM | 528 | CE1 | HIS | 68 | 15.030 | 16.185 | 48.403 | 1.00 | 13.50 |
| ATOM | 529 | NE2 | HIS | 68 | 14.461 | 16.217 | 47.211 | 1.00 | 14.36 |
| ATOM | 530 | C | HIS | 68 | 10.771 | 13.147 | 50.722 | 1.00 | 14.18 |
| ATOM | 531 | O | HIS | 68 | 11.002 | 13.282 | 51.929 | 1.00 | 12.96 |
| ATOM | 532 | N | THR | 69 | 9.631 | 12.651 | 50.247 | 1.00 | 13.76 |
| ATOM | 533 | CA | THR | 69 | 8.572 | 12.193 | 51.125 | 1.00 | 12.86 |
| ATOM | 534 | CB | THR | 69 | 7.291 | 11.816 | 50.321 | 1.00 | 13.67 |
| ATOM | 535 | OG1 | THR | 69 | 6.692 | 13.004 | 49.794 | 1.00 | 13.10 |
| ATOM | 536 | CG2 | THR | 69 | 6.290 | 11.098 | 51.200 | 1.00 | 13.21 |
| ATOM | 537 | C | THR | 69 | 9.080 | 10.957 | 51.877 | 1.00 | 12.94 |
| ATOM | 538 | O | THR | 69 | 8.891 | 10.837 | 53.086 | 1.00 | 13.96 |
| ATOM | 539 | N | ALA | 70 | 9.736 | 10.045 | 51.166 | 1.00 | 12.91 |
| ATOM | 540 | CA | ALA | 70 | 10.266 | 8.840 | 51.795 | 1.00 | 12.85 |
| ATOM | 541 | CB | ALA | 70 | 10.905 | 7.924 | 50.743 | 1.00 | 16.07 |
| ATOM | 542 | C | ALA | 70 | 11.285 | 9.166 | 52.893 | 1.00 | 13.23 |
| ATOM | 543 | O | ALA | 70 | 11.278 | 8.543 | 53.959 | 1.00 | 12.96 |
| ATOM | 544 | N | ALA | 71 | 12.157 | 10.136 | 52.635 | 1.00 | 13.50 |
| ATOM | 545 | CA | ALA | 71 | 13.174 | 10.519 | 53.613 | 1.00 | 13.45 |
| ATOM | 546 | CB | ALA | 71 | 14.166 | 11.480 | 52.984 | 1.00 | 14.53 |
| ATOM | 547 | C | ALA | 71 | 12.539 | 11.153 | 54.845 | 1.00 | 14.10 |
| ATOM | 548 | O | ALA | 71 | 12.935 | 10.876 | 55.981 | 1.00 | 14.47 |
| ATOM | 549 | N | VAL | 72 | 11.542 | 11.999 | 54.622 | 1.00 | 14.55 |
| ATOM | 550 | CA | VAL | 72 | 10.861 | 12.654 | 55.736 | 1.00 | 15.46 |
| ATOM | 551 | CB | VAL | 72 | 9.830 | 13.682 | 55.227 | 1.00 | 16.14 |
| ATOM | 552 | CG1 | VAL | 72 | 9.000 | 14.217 | 56.394 | 1.00 | 16.03 |
| ATOM | 553 | CG2 | VAL | 72 | 10.540 | 14.808 | 54.509 | 1.00 | 16.34 |
| ATOM | 554 | C | VAL | 72 | 10.153 | 11.606 | 56.590 | 1.00 | 15.16 |
| ATOM | 555 | O | VAL | 72 | 10.183 | 11.668 | 57.824 | 1.00 | 14.69 |
| ATOM | 556 | N | ARG | 73 | 9.522 | 10.633 | 55.936 | 1.00 | 14.57 |
| ATOM | 557 | CA | ARG | 73 | 8.815 | 9.593 | 56.669 | 1.00 | 12.67 |
| ATOM | 558 | CB | ARG | 73 | 8.060 | 8.654 | 55.719 | 1.00 | 13.42 |
| ATOM | 559 | CG | ARG | 73 | 7.356 | 7.508 | 56.447 | 1.00 | 12.53 |
| ATOM | 560 | CD | ARG | 73 | 6.380 | 8.015 | 57.520 | 1.00 | 16.45 |
| ATOM | 561 | NE | ARG | 73 | 5.237 | 8.713 | 56.933 | 1.00 | 16.27 |
| ATOM | 562 | CZ | ARG | 73 | 4.358 | 9.437 | 57.617 | 1.00 | 17.72 |
| ATOM | 563 | NH1 | ARG | 73 | 4.479 | 9.573 | 58.934 | 1.00 | 13.94 |
| ATOM | 564 | NH2 | ARG | 73 | 3.353 | 10.027 | 56.982 | 1.00 | 16.25 |
| ATOM | 565 | C | ARG | 73 | 9.748 | 8.767 | 57.540 | 1.00 | 12.87 |
| ATOM | 566 | O | ARG | 73 | 9.369 | 8.343 | 58.631 | 1.00 | 13.64 |
| ATOM | 567 | N | ARG | 74 | 10.963 | 8.524 | 57.057 | 1.00 | 14.65 |
| ATOM | 568 | CA | ARG | 74 | 11.926 | 7.738 | 57.823 | 1.00 | 13.53 |
| ATOM | 569 | CB | ARG | 74 | 13.155 | 7.405 | 56.975 | 1.00 | 15.28 |
| ATOM | 570 | CG | ARG | 74 | 12.860 | 6.543 | 55.752 | 1.00 | 16.51 |
| ATOM | 571 | CD | ARG | 74 | 14.133 | 5.937 | 55.155 | 1.00 | 17.73 |
| ATOM | 572 | NE | ARG | 74 | 13.895 | 5.341 | 53.838 | 1.00 | 20.82 |
| ATOM | 573 | CZ | ARG | 74 | 13.896 | 6.020 | 52.694 | 1.00 | 22.35 |
| ATOM | 574 | NH1 | ARG | 74 | 14.127 | 7.325 | 52.688 | 1.00 | 23.90 |
| ATOM | 575 | NH2 | ARG | 74 | 13.656 | 5.397 | 51.552 | 1.00 | 27.63 |
| ATOM | 576 | C | ARG | 74 | 12.355 | 8.513 | 59.052 | 1.00 | 16.13 |
| ATOM | 577 | O | ARG | 74 | 12.673 | 7.932 | 60.093 | 1.00 | 16.86 |
| ATOM | 578 | N | GLY | 75 | 12.359 | 9.834 | 58.923 | 1.00 | 16.62 |
| ATOM | 579 | CA | GLY | 75 | 12.753 | 10.681 | 60.035 | 1.00 | 17.65 |
| ATOM | 580 | C | GLY | 75 | 11.629 | 10.935 | 61.019 | 1.00 | 17.81 |
| ATOM | 581 | O | GLY | 75 | 11.865 | 11.107 | 62.215 | 1.00 | 17.98 |
| ATOM | 582 | N | ALA | 76 | 10.398 | 10.952 | 60.525 | 1.00 | 17.56 |
| ATOM | 583 | CA | ALA | 76 | 9.240 | 11.210 | 61.385 | 1.00 | 17.19 |
| ATOM | 584 | CB | ALA | 76 | 8.767 | 12.640 | 61.173 | 1.00 | 16.95 |
| ATOM | 585 | C | ALA | 76 | 8.108 | 10.229 | 61.094 | 1.00 | 18.56 |
| ATOM | 586 | O | ALA | 76 | 7.100 | 10.590 | 60.492 | 1.00 | 18.58 |
| ATOM | 587 | N | PRO | 77 | 8.255 | 8.976 | 61.549 | 1.00 | 20.24 |
| ATOM | 588 | CD | PRO | 77 | 9.361 | 8.507 | 62.400 | 1.00 | 21.81 |
| ATOM | 589 | CA | PRO | 77 | 7.271 | 7.908 | 61.348 | 1.00 | 20.91 |
| ATOM | 590 | CB | PRO | 77 | 7.949 | 6.698 | 61.982 | 1.00 | 22.03 |
| ATOM | 591 | CG | PRO | 77 | 8.749 | 7.303 | 63.072 | 1.00 | 25.30 |
| ATOM | 592 | C | PRO | 77 | 5.870 | 8.138 | 61.902 | 1.00 | 20.81 |
| ATOM | 593 | O | PRO | 77 | 4.929 | 7.462 | 61.488 | 1.00 | 20.60 |
| ATOM | 594 | N | ASN | 78 | 5.720 | 9.080 | 62.828 | 1.00 | 19.26 |
| ATOM | 595 | CA | ASN | 78 | 4.402 | 9.342 | 63.399 | 1.00 | 20.69 |
| ATOM | 596 | CB | ASN | 78 | 4.449 | 9.242 | 64.932 | 1.00 | 22.75 |
| ATOM | 597 | CG | ASN | 78 | 4.554 | 7.806 | 65.414 | 1.00 | 27.00 |
| ATOM | 598 | OD1 | ASN | 78 | 3.772 | 6.951 | 65.001 | 1.00 | 31.16 |
| ATOM | 599 | ND2 | ASN | 78 | 5.515 | 7.534 | 66.287 | 1.00 | 29.98 |
| ATOM | 600 | C | ASN | 78 | 3.821 | 10.693 | 62.992 | 1.00 | 19.38 |
| ATOM | 601 | O | ASN | 78 | 2.750 | 11.076 | 63.456 | 1.00 | 21.43 |
| ATOM | 602 | N | CYS | 79 | 4.507 | 11.399 | 62.104 | 1.00 | 17.77 |
| ATOM | 603 | CA | CYS | 79 | 4.040 | 12.713 | 61.686 | 1.00 | 19.13 |
| ATOM | 604 | CB | CYS | 79 | 5.210 | 13.553 | 61.150 | 1.00 | 19.79 |
| ATOM | 605 | SG | CYS | 79 | 5.646 | 13.275 | 59.389 | 1.00 | 24.05 |
| ATOM | 606 | C | CYS | 79 | 2.938 | 12.667 | 60.633 | 1.00 | 19.96 |
| ATOM | 607 | O | CYS | 79 | 2.735 | 11.652 | 59.961 | 1.00 | 17.94 |
| ATOM | 608 | N | LEU | 80 | 2.202 | 13.770 | 60.528 | 1.00 | 19.53 |
| ATOM | 609 | CA | LEU | 80 | 1.163 | 13.904 | 59.519 | 1.00 | 19.26 |
| ATOM | 610 | CB | LEU | 80 | 0.054 | 14.860 | 59.978 | 1.00 | 20.96 |
| ATOM | 611 | CG | LEU | 80 | −0.984 | 15.225 | 58.909 | 1.00 | 21.27 |
| ATOM | 612 | CD1 | LEU | 80 | −1.670 | 13.967 | 58.396 | 1.00 | 22.20 |
| ATOM | 613 | CD2 | LEU | 80 | −2.008 | 16.195 | 59.488 | 1.00 | 22.16 |
| ATOM | 614 | C | LEU | 80 | 1.962 | 14.527 | 58.387 | 1.00 | 18.90 |
| ATOM | 615 | O | LEU | 80 | 2.442 | 15.655 | 58.509 | 1.00 | 18.11 |
| ATOM | 616 | N | LEU | 81 | 2.120 | 13.788 | 57.294 | 1.00 | 15.94 |
| ATOM | 617 | CA | LEU | 81 | 2.924 | 14.261 | 56.181 | 1.00 | 16.28 |
| ATOM | 618 | CB | LEU | 81 | 3.930 | 13.169 | 55.775 | 1.00 | 18.46 |
| ATOM | 619 | CG | LEU | 81 | 5.213 | 13.532 | 55.008 | 1.00 | 18.26 |
| ATOM | 620 | CD1 | LEU | 81 | 6.102 | 12.304 | 54.937 | 1.00 | 18.48 |
| ATOM | 621 | CD2 | LEU | 81 | 4.884 | 14.038 | 53.605 | 1.00 | 21.41 |
| ATOM | 622 | C | LEU | 81 | 2.090 | 14.669 | 54.986 | 1.00 | 16.06 |
| ATOM | 623 | O | LEU | 81 | 1.357 | 13.857 | 54.417 | 1.00 | 16.48 |
| ATOM | 624 | N | LEU | 82 | 2.183 | 15.944 | 54.628 | 1.00 | 16.31 |
| ATOM | 625 | CA | LEU | 82 | 1.457 | 16.466 | 53.477 | 1.00 | 17.30 |
| ATOM | 626 | CB | LEU | 82 | 0.897 | 17.859 | 53.766 | 1.00 | 19.25 |
| ATOM | 627 | CG | LEU | 82 | −0.451 | 17.985 | 54.495 | 1.00 | 22.19 |
| ATOM | 628 | CD1 | LEU | 82 | −0.449 | 17.200 | 55.792 | 1.00 | 21.32 |
| ATOM | 629 | CD2 | LEU | 82 | −0.720 | 19.462 | 54.750 | 1.00 | 21.16 |
| ATOM | 630 | C | LEU | 82 | 2.458 | 16.557 | 52.342 | 1.00 | 17.89 |
| ATOM | 631 | O | LEU | 82 | 3.560 | 17.068 | 52.531 | 1.00 | 18.88 |
| ATOM | 632 | N | ALA | 83 | 2.092 | 16.053 | 51.171 | 1.00 | 16.82 |
| ATOM | 633 | CA | ALA | 83 | 2.997 | 16.114 | 50.033 | 1.00 | 16.39 |
| ATOM | 634 | CB | ALA | 83 | 3.406 | 14.720 | 49.607 | 1.00 | 16.49 |
| ATOM | 635 | C | ALA | 83 | 2.337 | 16.841 | 48.881 | 1.00 | 14.35 |
| ATOM | 636 | O | ALA | 83 | 1.186 | 16.579 | 48.554 | 1.00 | 14.15 |
| ATOM | 637 | N | ASP | 84 | 3.058 | 17.775 | 48.274 | 1.00 | 13.95 |
| ATOM | 638 | CA | ASP | 84 | 2.498 | 18.505 | 47.148 | 1.00 | 16.00 |
| ATOM | 639 | CB | ASP | 84 | 3.268 | 19.799 | 46.893 | 1.00 | 22.80 |
| ATOM | 640 | CG | ASP | 84 | 2.767 | 20.957 | 47.723 | 1.00 | 24.54 |
| ATOM | 641 | OD1 | ASP | 84 | 1.548 | 21.195 | 47.752 | 1.00 | 30.38 |
| ATOM | 642 | OD2 | ASP | 84 | 3.602 | 21.644 | 48.329 | 1.00 | 32.60 |
| ATOM | 643 | C | ASP | 84 | 2.526 | 17.716 | 45.851 | 1.00 | 16.16 |
| ATOM | 644 | O | ASP | 84 | 3.400 | 16.875 | 45.640 | 1.00 | 14.13 |
| ATOM | 645 | N | LEU | 85 | 1.535 | 17.967 | 44.998 | 1.00 | 15.10 |
| ATOM | 646 | CA | LEU | 85 | 1.539 | 17.381 | 43.667 | 1.00 | 15.19 |
| ATOM | 647 | CB | LEU | 85 | 0.127 | 17.043 | 43.167 | 1.00 | 16.96 |
| ATOM | 648 | CG | LEU | 85 | −0.451 | 15.751 | 43.751 | 1.00 | 17.98 |
| ATOM | 649 | CD1 | LEU | 85 | −1.753 | 15.360 | 43.028 | 1.00 | 17.36 |
| ATOM | 650 | CD2 | LEU | 85 | 0.574 | 14.639 | 43.604 | 1.00 | 17.58 |
| ATOM | 651 | C | LEU | 85 | 2.111 | 18.593 | 42.937 | 1.00 | 14.95 |
| ATOM | 652 | O | LEU | 85 | 1.563 | 19.694 | 43.024 | 1.00 | 15.92 |
| ATOM | 653 | N | PRO | 86 | 3.255 | 18.419 | 42.262 | 1.00 | 14.05 |
| ATOM | 654 | CD | PRO | 86 | 3.984 | 17.143 | 42.170 | 1.00 | 15.98 |
| ATOM | 655 | CA | PRO | 86 | 3.949 | 19.473 | 41.518 | 1.00 | 15.84 |
| ATOM | 656 | CB | PRO | 86 | 5.306 | 18.839 | 41.224 | 1.00 | 16.60 |
| ATOM | 657 | CG | PRO | 86 | 4.933 | 17.386 | 41.018 | 1.00 | 16.94 |
| ATOM | 658 | C | PRO | 86 | 3.249 | 19.970 | 40.255 | 1.00 | 17.90 |
| ATOM | 659 | O | PRO | 86 | 2.161 | 19.515 | 39.899 | 1.00 | 17.49 |
| ATOM | 660 | N | PHE | 87 | 3.897 | 20.917 | 39.591 | 1.00 | 17.02 |
| ATOM | 661 | CA | PHE | 87 | 3.386 | 21.509 | 38.371 | 1.00 | 18.00 |
| ATOM | 662 | CB | PHE | 87 | 4.486 | 22.357 | 37.728 | 1.00 | 19.87 |
| ATOM | 663 | CG | PHE | 87 | 4.125 | 22.906 | 36.383 | 1.00 | 19.63 |
| ATOM | 664 | CD1 | PHE | 87 | 3.025 | 23.739 | 36.229 | 1.00 | 19.99 |
| ATOM | 665 | CD2 | PHE | 87 | 4.893 | 22.588 | 35.265 | 1.00 | 20.71 |
| ATOM | 666 | CE1 | PHE | 87 | 2.692 | 24.249 | 34.982 | 1.00 | 21.55 |
| ATOM | 667 | CE2 | PHE | 87 | 4.567 | 23.095 | 34.012 | 1.00 | 20.03 |
| ATOM | 668 | CZ | PHE | 87 | 3.466 | 23.926 | 33.870 | 1.00 | 21.84 |
| ATOM | 669 | C | PHE | 87 | 2.871 | 20.467 | 37.373 | 1.00 | 17.52 |
| ATOM | 670 | O | PHE | 87 | 3.561 | 19.495 | 37.051 | 1.00 | 16.53 |
| ATOM | 671 | N | MET | 88 | 1.644 | 20.682 | 36.909 | 1.00 | 16.65 |
| ATOM | 672 | CA | MET | 88 | 0.984 | 19.816 | 35.936 | 1.00 | 18.63 |
| ATOM | 673 | CB | MET | 88 | 1.666 | 19.944 | 34.575 | 1.00 | 20.77 |
| ATOM | 674 | CG | MET | 88 | 0.767 | 19.578 | 33.413 | 1.00 | 22.54 |
| ATOM | 675 | SD | MET | 88 | −0.593 | 20.732 | 33.216 | 1.00 | 21.14 |
| ATOM | 676 | CE | MET | 88 | 0.111 | 21.920 | 32.099 | 1.00 | 22.91 |
| ATOM | 677 | C | MET | 88 | 0.931 | 18.340 | 36.326 | 1.00 | 19.33 |
| ATOM | 678 | O | MET | 88 | 0.987 | 17.463 | 35.461 | 1.00 | 22.28 |
| ATOM | 679 | N | ALA | 89 | 0.822 | 18.062 | 37.619 | 1.00 | 17.87 |
| ATOM | 680 | CA | ALA | 89 | 0.749 | 16.685 | 38.086 | 1.00 | 17.24 |
| ATOM | 681 | CB | ALA | 89 | 1.609 | 16.506 | 39.333 | 1.00 | 18.47 |
| ATOM | 682 | C | ALA | 89 | −0.701 | 16.285 | 38.379 | 1.00 | 16.90 |
| ATOM | 683 | O | ALA | 89 | −0.978 | 15.164 | 38.816 | 1.00 | 17.43 |
| ATOM | 684 | N | TYR | 90 | −1.624 | 17.209 | 38.145 | 1.00 | 16.10 |
| ATOM | 685 | CA | TYR | 90 | −3.041 | 16.942 | 38.364 | 1.00 | 15.59 |
| ATOM | 686 | CB | TYR | 90 | −3.452 | 17.350 | 39.790 | 1.00 | 14.88 |
| ATOM | 687 | CG | TYR | 90 | −2.959 | 18.715 | 40.223 | 1.00 | 15.60 |
| ATOM | 688 | CD1 | TYR | 90 | −3.753 | 19.854 | 40.064 | 1.00 | 17.89 |
| ATOM | 689 | CE1 | TYR | 90 | −3.288 | 21.117 | 40.454 | 1.00 | 17.95 |
| ATOM | 690 | CD2 | TYR | 90 | −1.690 | 18.870 | 40.782 | 1.00 | 17.54 |
| ATOM | 691 | CE2 | TYR | 90 | −1.217 | 20.122 | 41.173 | 1.00 | 17.49 |
| ATOM | 692 | CZ | TYR | 90 | −2.016 | 21.235 | 41.008 | 1.00 | 18.23 |
| ATOM | 693 | OH | TYR | 90 | −1.543 | 22.470 | 41.404 | 1.00 | 19.18 |
| ATOM | 694 | C | TYR | 90 | −3.885 | 17.666 | 37.322 | 1.00 | 15.82 |
| ATOM | 695 | O | TYR | 90 | −4.937 | 18.225 | 37.628 | 1.00 | 17.20 |
| ATOM | 696 | N | ALA | 91 | −3.412 | 17.628 | 36.079 | 1.00 | 16.74 |
| ATOM | 697 | CA | ALA | 91 | −4.085 | 18.272 | 34.959 | 1.00 | 17.95 |
| ATOM | 698 | CB | ALA | 91 | −3.177 | 18.262 | 33.731 | 1.00 | 18.19 |
| ATOM | 699 | C | ALA | 91 | −5.425 | 17.611 | 34.631 | 1.00 | 17.59 |
| ATOM | 700 | O | ALA | 91 | −6.289 | 18.230 | 34.010 | 1.00 | 16.62 |
| ATOM | 701 | N | THR | 92 | −5.580 | 16.347 | 35.015 | 1.00 | 16.91 |
| ATOM | 702 | CA | THR | 92 | −6.838 | 15.619 | 34.811 | 1.00 | 15.64 |
| ATOM | 703 | CB | THR | 92 | −6.821 | 14.689 | 33.566 | 1.00 | 17.31 |
| ATOM | 704 | OG1 | THR | 92 | −5.942 | 13.582 | 33.804 | 1.00 | 15.34 |
| ATOM | 705 | CG2 | THR | 92 | −6.369 | 15.438 | 32.322 | 1.00 | 15.98 |
| ATOM | 706 | C | THR | 92 | −7.052 | 14.720 | 36.021 | 1.00 | 16.55 |
| ATOM | 707 | O | THR | 92 | −6.097 | 14.366 | 36.711 | 1.00 | 17.07 |
| ATOM | 708 | N | PRO | 93 | −8.310 | 14.339 | 36.299 | 1.00 | 16.42 |
| ATOM | 709 | CD | PRO | 93 | −9.570 | 14.771 | 35.669 | 1.00 | 17.40 |
| ATOM | 710 | CA | PRO | 93 | −8.568 | 13.470 | 37.447 | 1.00 | 15.13 |
| ATOM | 711 | CB | PRO | 93 | −10.056 | 13.168 | 37.312 | 1.00 | 17.06 |
| ATOM | 712 | CG | PRO | 93 | −10.589 | 14.464 | 36.750 | 1.00 | 16.77 |
| ATOM | 713 | C | PRO | 93 | −7.696 | 12.210 | 37.411 | 1.00 | 16.09 |
| ATOM | 714 | O | PRO | 93 | −7.028 | 11.879 | 38.396 | 1.00 | 13.79 |
| ATOM | 715 | N | GLU | 94 | −7.689 | 11.517 | 36.273 | 1.00 | 16.39 |
| ATOM | 716 | CA | GLU | 94 | −6.882 | 10.305 | 36.120 | 1.00 | 17.29 |
| ATOM | 717 | CB | GLU | 94 | −6.948 | 9.791 | 34.680 | 1.00 | 20.57 |
| ATOM | 718 | CG | GLU | 94 | −8.040 | 8.779 | 34.426 | 1.00 | 27.03 |
| ATOM | 719 | CD | GLU | 94 | −7.968 | 8.209 | 33.024 | 1.00 | 30.80 |
| ATOM | 720 | OE1 | GLU | 94 | −6.908 | 7.660 | 32.659 | 1.00 | 34.96 |
| ATOM | 721 | OE2 | GLU | 94 | −8.965 | 8.309 | 32.285 | 1.00 | 36.52 |
| ATOM | 722 | C | GLU | 94 | −5.418 | 10.492 | 36.497 | 1.00 | 17.89 |
| ATOM | 723 | O | GLU | 94 | −4.846 | 9.658 | 37.194 | 1.00 | 16.77 |
| ATOM | 724 | N | GLN | 95 | −4.806 | 11.573 | 36.029 | 1.00 | 15.83 |
| ATOM | 725 | CA | GLN | 95 | −3.408 | 11.811 | 36.350 | 1.00 | 16.28 |
| ATOM | 726 | CB | GLN | 95 | −2.845 | 12.932 | 35.491 | 1.00 | 18.34 |
| ATOM | 727 | CG | GLN | 95 | −2.936 | 12.662 | 34.002 | 1.00 | 24.91 |
| ATOM | 728 | CD | GLN | 95 | −2.424 | 13.826 | 33.189 | 1.00 | 29.82 |
| ATOM | 729 | OE1 | GLN | 95 | −1.215 | 14.043 | 33.081 | 1.00 | 31.53 |
| ATOM | 730 | NE2 | GLN | 95 | −3.347 | 14.606 | 32.632 | 1.00 | 31.62 |
| ATOM | 731 | C | GLN | 95 | −3.232 | 12.144 | 37.817 | 1.00 | 13.83 |
| ATOM | 732 | O | GLN | 95 | −2.245 | 11.743 | 38.422 | 1.00 | 14.18 |
| ATOM | 733 | N | ALA | 96 | −4.173 | 12.888 | 38.393 | 1.00 | 12.99 |
| ATOM | 734 | CA | ALA | 96 | −4.071 | 13.213 | 39.813 | 1.00 | 11.89 |
| ATOM | 735 | CB | ALA | 96 | −5.229 | 14.113 | 40.243 | 1.00 | 10.87 |
| ATOM | 736 | C | ALA | 96 | −4.090 | 11.911 | 40.611 | 1.00 | 12.60 |
| ATOM | 737 | O | ALA | 96 | −3.311 | 11.746 | 41.549 | 1.00 | 11.80 |
| ATOM | 738 | N | PHE | 97 | −4.970 | 10.979 | 40.236 | 1.00 | 12.82 |
| ATOM | 739 | CA | PHE | 97 | −5.050 | 9.709 | 40.956 | 1.00 | 13.08 |
| ATOM | 740 | CB | PHE | 97 | −6.072 | 8.741 | 40.332 | 1.00 | 13.33 |
| ATOM | 741 | CG | PHE | 97 | −7.459 | 9.303 | 40.173 | 1.00 | 14.06 |
| ATOM | 742 | CD1 | PHE | 97 | −7.975 | 10.220 | 41.079 | 1.00 | 14.07 |
| ATOM | 743 | CD2 | PHE | 97 | −8.254 | 8.891 | 39.113 | 1.00 | 13.38 |
| ATOM | 744 | CE1 | PHE | 97 | −9.273 | 10.725 | 40.931 | 1.00 | 11.22 |
| ATOM | 745 | CE2 | PHE | 97 | −9.556 | 9.385 | 38.948 | 1.00 | 13.40 |
| ATOM | 746 | CZ | PHE | 97 | −10.061 | 10.302 | 39.859 | 1.00 | 11.20 |
| ATOM | 747 | C | PHE | 97 | −3.699 | 8.990 | 40.975 | 1.00 | 13.62 |
| ATOM | 748 | O | PHE | 97 | −3.244 | 8.552 | 42.026 | 1.00 | 13.09 |
| ATOM | 749 | N | GLU | 98 | −3.064 | 8.856 | 39.815 | 1.00 | 14.92 |
| ATOM | 750 | CA | GLU | 98 | −1.786 | 8.154 | 39.768 | 1.00 | 16.75 |
| ATOM | 751 | CB | GLU | 98 | −1.356 | 7.859 | 38.327 | 1.00 | 20.44 |
| ATOM | 752 | CG | GLU | 98 | −0.045 | 7.064 | 38.268 | 1.00 | 28.53 |
| ATOM | 753 | CD | GLU | 98 | −0.113 | 5.768 | 39.066 | 1.00 | 33.54 |
| ATOM | 754 | OE1 | GLU | 98 | −0.767 | 4.810 | 38.587 | 1.00 | 35.57 |
| ATOM | 755 | OE2 | GLU | 98 | 0.472 | 5.706 | 40.187 | 1.00 | 34.76 |
| ATOM | 756 | C | GLU | 98 | −0.660 | 8.888 | 40.480 | 1.00 | 14.28 |
| ATOM | 757 | O | GLU | 98 | 0.134 | 8.275 | 41.198 | 1.00 | 14.65 |
| ATOM | 758 | N | ASN | 99 | −0.580 | 10.196 | 40.294 | 1.00 | 12.45 |
| ATOM | 759 | CA | ASN | 99 | 0.490 | 10.944 | 40.941 | 1.00 | 11.69 |
| ATOM | 760 | CB | ASN | 99 | 0.627 | 12.329 | 40.299 | 1.00 | 11.89 |
| ATOM | 761 | CG | ASN | 99 | 1.172 | 12.238 | 38.890 | 1.00 | 12.59 |
| ATOM | 762 | OD1 | ASN | 99 | 2.019 | 11.384 | 38.609 | 1.00 | 13.02 |
| ATOM | 763 | ND2 | ASN | 99 | 0.707 | 13.105 | 38.002 | 1.00 | 13.56 |
| ATOM | 764 | C | ASN | 99 | 0.286 | 11.026 | 42.451 | 1.00 | 13.55 |
| ATOM | 765 | O | ASN | 99 | 1.256 | 10.953 | 43.216 | 1.00 | 11.87 |
| ATOM | 766 | N | ALA | 100 | −0.970 | 11.157 | 42.879 | 1.00 | 12.15 |
| ATOM | 767 | CA | ALA | 100 | −1.284 | 11.189 | 44.306 | 1.00 | 14.14 |
| ATOM | 768 | CB | ALA | 100 | −2.777 | 11.489 | 44.531 | 1.00 | 13.05 |
| ATOM | 769 | C | ALA | 100 | −0.940 | 9.814 | 44.878 | 1.00 | 12.60 |
| ATOM | 770 | O | ALA | 100 | −0.347 | 9.709 | 45.953 | 1.00 | 13.66 |
| ATOM | 771 | N | ALA | 101 | −1.312 | 8.748 | 44.175 | 1.00 | 12.06 |
| ATOM | 772 | CA | ALA | 101 | −0.999 | 7.412 | 44.679 | 1.00 | 10.87 |
| ATOM | 773 | CB | ALA | 101 | −1.590 | 6.338 | 43.755 | 1.00 | 10.90 |
| ATOM | 774 | C | ALA | 101 | 0.517 | 7.204 | 44.853 | 1.00 | 10.31 |
| ATOM | 775 | O | ALA | 101 | 0.953 | 6.543 | 45.794 | 1.00 | 12.24 |
| ATOM | 776 | N | THR | 102 | 1.322 | 7.766 | 43.958 | 1.00 | 10.77 |
| ATOM | 777 | CA | THR | 102 | 2.781 | 7.606 | 44.046 | 1.00 | 10.62 |
| ATOM | 778 | CB | THR | 102 | 3.450 | 8.244 | 42.833 | 1.00 | 11.82 |
| ATOM | 779 | OG1 | THR | 102 | 3.011 | 7.556 | 41.648 | 1.00 | 12.54 |
| ATOM | 780 | CG2 | THR | 102 | 4.965 | 8.143 | 42.934 | 1.00 | 13.14 |
| ATOM | 781 | C | THR | 102 | 3.329 | 8.218 | 45.331 | 1.00 | 12.67 |
| ATOM | 782 | O | THR | 102 | 4.122 | 7.609 | 46.053 | 1.00 | 13.26 |
| ATOM | 783 | N | VAL | 103 | 2.872 | 9.430 | 45.608 | 1.00 | 13.06 |
| ATOM | 784 | CA | VAL | 103 | 3.275 | 10.178 | 46.786 | 1.00 | 17.15 |
| ATOM | 785 | CB | VAL | 103 | 2.771 | 11.622 | 46.644 | 1.00 | 20.15 |
| ATOM | 786 | CG1 | VAL | 103 | 2.563 | 12.250 | 47.990 | 1.00 | 26.49 |
| ATOM | 787 | CG2 | VAL | 103 | 3.758 | 12.408 | 45.807 | 1.00 | 21.10 |
| ATOM | 788 | C | VAL | 103 | 2.769 | 9.533 | 48.084 | 1.00 | 16.61 |
| ATOM | 789 | O | VAL | 103 | 3.477 | 9.519 | 49.097 | 1.00 | 14.86 |
| ATOM | 790 | N | MET | 104 | 1.554 | 8.989 | 48.048 | 1.00 | 13.98 |
| ATOM | 791 | CA | MET | 104 | 0.976 | 8.336 | 49.219 | 1.00 | 13.90 |
| ATOM | 792 | CB | MET | 104 | −0.514 | 8.053 | 48.997 | 1.00 | 16.23 |
| ATOM | 793 | CG | MET | 104 | −1.373 | 9.288 | 48.838 | 1.00 | 20.48 |
| ATOM | 794 | SD | MET | 104 | −1.516 | 10.232 | 50.350 | 1.00 | 23.82 |
| ATOM | 795 | CE | MET | 104 | −2.587 | 9.130 | 51.315 | 1.00 | 23.65 |
| ATOM | 796 | C | MET | 104 | 1.701 | 7.031 | 49.537 | 1.00 | 13.22 |
| ATOM | 797 | O | MET | 104 | 1.979 | 6.735 | 50.707 | 1.00 | 13.56 |
| ATOM | 798 | N | ARG | 105 | 2.008 | 6.242 | 48.508 | 1.00 | 13.42 |
| ATOM | 799 | CA | ARG | 105 | 2.711 | 4.989 | 48.743 | 1.00 | 11.59 |
| ATOM | 800 | CB | ARG | 105 | 2.817 | 4.152 | 47.453 | 1.00 | 14.28 |
| ATOM | 801 | CG | ARG | 105 | 1.492 | 3.637 | 46.914 | 1.00 | 15.18 |
| ATOM | 802 | CD | ARG | 105 | 1.673 | 2.542 | 45.848 | 1.00 | 15.37 |
| ATOM | 803 | NE | ARG | 105 | 0.436 | 2.355 | 45.086 | 1.00 | 18.07 |
| ATOM | 804 | CZ | ARG | 105 | 0.151 | 2.986 | 43.951 | 1.00 | 16.17 |
| ATOM | 805 | NH1 | ARG | 105 | 1.021 | 3.837 | 43.420 | 1.00 | 14.58 |
| ATOM | 806 | NH2 | ARG | 105 | −1.030 | 2.802 | 43.376 | 1.00 | 16.92 |
| ATOM | 807 | C | ARG | 105 | 4.112 | 5.296 | 49.275 | 1.00 | 10.65 |
| ATOM | 808 | O | ARG | 105 | 4.684 | 4.494 | 50.009 | 1.00 | 12.75 |
| ATOM | 809 | N | ALA | 106 | 4.645 | 6.463 | 48.916 | 1.00 | 12.09 |
| ATOM | 810 | CA | ALA | 106 | 5.978 | 6.873 | 49.346 | 1.00 | 13.06 |
| ATOM | 811 | CB | ALA | 106 | 6.496 | 7.999 | 48.455 | 1.00 | 14.76 |
| ATOM | 812 | C | ALA | 106 | 6.015 | 7.305 | 50.807 | 1.00 | 15.39 |
| ATOM | 813 | O | ALA | 106 | 7.094 | 7.529 | 51.365 | 1.00 | 15.17 |
| ATOM | 814 | N | GLY | 107 | 4.841 | 7.430 | 51.420 | 1.00 | 14.50 |
| ATOM | 815 | CA | GLY | 107 | 4.779 | 7.806 | 52.821 | 1.00 | 14.87 |
| ATOM | 816 | C | GLY | 107 | 3.904 | 8.991 | 53.207 | 1.00 | 15.59 |
| ATOM | 817 | O | GLY | 107 | 3.751 | 9.273 | 54.393 | 1.00 | 16.79 |
| ATOM | 818 | N | ALA | 108 | 3.331 | 9.691 | 52.238 | 1.00 | 13.12 |
| ATOM | 819 | CA | ALA | 108 | 2.484 | 10.833 | 52.569 | 1.00 | 13.97 |
| ATOM | 820 | CB | ALA | 108 | 2.307 | 11.724 | 51.342 | 1.00 | 14.04 |
| ATOM | 821 | C | ALA | 108 | 1.122 | 10.374 | 53.099 | 1.00 | 14.46 |
| ATOM | 822 | O | ALA | 108 | 0.696 | 9.251 | 52.834 | 1.00 | 14.12 |
| ATOM | 823 | N | ASN | 109 | 0.461 | 11.230 | 53.880 | 1.00 | 14.94 |
| ATOM | 824 | CA | ASN | 109 | −0.863 | 10.919 | 54.440 | 1.00 | 14.90 |
| ATOM | 825 | CB | ASN | 109 | −0.947 | 11.277 | 55.931 | 1.00 | 14.17 |
| ATOM | 826 | CG | ASN | 109 | 0.012 | 10.493 | 56.790 | 1.00 | 14.97 |
| ATOM | 827 | OD1 | ASN | 109 | −0.101 | 9.276 | 56.926 | 1.00 | 16.84 |
| ATOM | 828 | ND2 | ASN | 109 | 0.959 | 11.193 | 57.388 | 1.00 | 14.33 |
| ATOM | 829 | C | ASN | 109 | −1.901 | 11.781 | 53.737 | 1.00 | 13.58 |
| ATOM | 830 | O | ASN | 109 | −3.102 | 11.556 | 53.870 | 1.00 | 15.82 |
| ATOM | 831 | N | MET | 110 | −1.427 | 12.768 | 52.991 | 1.00 | 13.54 |
| ATOM | 832 | CA | MET | 110 | −2.316 | 13.704 | 52.332 | 1.00 | 14.31 |
| ATOM | 833 | CB | MET | 110 | −2.828 | 14.688 | 53.394 | 1.00 | 14.27 |
| ATOM | 834 | CG | MET | 110 | −3.595 | 15.902 | 52.910 | 1.00 | 19.21 |
| ATOM | 835 | SD | MET | 110 | −4.143 | 16.871 | 54.371 | 1.00 | 20.75 |
| ATOM | 836 | CE | MET | 110 | −5.845 | 16.364 | 54.495 | 1.00 | 18.93 |
| ATOM | 837 | C | MET | 110 | −1.576 | 14.431 | 51.219 | 1.00 | 13.50 |
| ATOM | 838 | O | MET | 110 | −0.358 | 14.635 | 51.285 | 1.00 | 14.01 |
| ATOM | 839 | N | VAL | 111 | −2.323 | 14.810 | 50.191 | 1.00 | 13.28 |
| ATOM | 840 | CA | VAL | 111 | −1.765 | 15.507 | 49.050 | 1.00 | 15.44 |
| ATOM | 841 | CB | VAL | 111 | −2.175 | 14.786 | 47.752 | 1.00 | 18.91 |
| ATOM | 842 | CG1 | VAL | 111 | −1.800 | 15.614 | 46.564 | 1.00 | 21.41 |
| ATOM | 843 | CG2 | VAL | 111 | −1.504 | 13.416 | 47.691 | 1.00 | 16.70 |
| ATOM | 844 | C | VAL | 111 | −2.271 | 16.944 | 49.000 | 1.00 | 15.84 |
| ATOM | 845 | O | VAL | 111 | −3.420 | 17.209 | 49.344 | 1.00 | 16.27 |
| ATOM | 846 | N | LYS | 112 | −1.411 | 17.869 | 48.586 | 1.00 | 18.00 |
| ATOM | 847 | CA | LYS | 112 | −1.810 | 19.266 | 48.475 | 1.00 | 17.62 |
| ATOM | 848 | CB | LYS | 112 | −0.912 | 20.174 | 49.324 | 1.00 | 16.94 |
| ATOM | 849 | CG | LYS | 112 | −1.299 | 21.650 | 49.203 | 1.00 | 18.28 |
| ATOM | 850 | CD | LYS | 112 | −0.768 | 22.491 | 50.354 | 1.00 | 20.19 |
| ATOM | 851 | CE | LYS | 112 | 0.738 | 22.675 | 50.288 | 1.00 | 21.39 |
| ATOM | 852 | NZ | LYS | 112 | 1.171 | 23.485 | 49.116 | 1.00 | 22.77 |
| ATOM | 853 | C | LYS | 112 | −1.738 | 19.707 | 47.022 | 1.00 | 18.52 |
| ATOM | 854 | O | LYS | 112 | −0.741 | 19.461 | 46.330 | 1.00 | 18.26 |
| ATOM | 855 | N | ILE | 113 | −2.800 | 20.360 | 46.563 | 1.00 | 18.70 |
| ATOM | 856 | CA | ILE | 113 | −2.878 | 20.836 | 45.187 | 1.00 | 19.67 |
| ATOM | 857 | CB | ILE | 113 | −3.852 | 19.967 | 44.359 | 1.00 | 20.73 |
| ATOM | 858 | CG2 | ILE | 113 | −3.270 | 18.573 | 44.169 | 1.00 | 20.69 |
| ATOM | 859 | CG1 | ILE | 113 | −5.201 | 19.870 | 45.067 | 1.00 | 21.18 |
| ATOM | 860 | CD1 | ILE | 113 | −6.238 | 19.041 | 44.316 | 1.00 | 22.35 |
| ATOM | 861 | C | ILE | 113 | −3.349 | 22.288 | 45.139 | 1.00 | 20.68 |
| ATOM | 862 | O | ILE | 113 | −4.206 | 22.694 | 45.919 | 1.00 | 23.09 |
| ATOM | 863 | N | GLU | 114 | −2.775 | 23.055 | 44.217 | 1.00 | 20.92 |
| ATOM | 864 | CA | GLU | 114 | −3.094 | 24.468 | 44.041 | 1.00 | 22.18 |
| ATOM | 865 | CB | GLU | 114 | −1.872 | 25.219 | 43.510 | 1.00 | 23.81 |
| ATOM | 866 | CG | GLU | 114 | −0.715 | 25.328 | 44.478 | 1.00 | 25.94 |
| ATOM | 867 | CD | GLU | 114 | 0.474 | 26.052 | 43.872 | 1.00 | 27.17 |
| ATOM | 868 | OE1 | GLU | 114 | 0.296 | 26.741 | 42.844 | 1.00 | 28.77 |
| ATOM | 869 | OE2 | GLU | 114 | 1.588 | 25.947 | 44.427 | 1.00 | 29.32 |
| ATOM | 870 | C | GLU | 114 | −4.247 | 24.707 | 43.074 | 1.00 | 23.10 |
| ATOM | 871 | O | GLU | 114 | −4.325 | 24.072 | 42.025 | 1.00 | 22.98 |
| ATOM | 872 | N | GLY | 115 | −5.135 | 25.636 | 43.420 | 1.00 | 22.55 |
| ATOM | 873 | CA | GLY | 115 | −6.246 | 25.933 | 42.534 | 1.00 | 24.88 |
| ATOM | 874 | C | GLY | 115 | −7.581 | 26.079 | 43.230 | 1.00 | 24.82 |
| ATOM | 875 | O | GLY | 115 | −7.720 | 25.743 | 44.406 | 1.00 | 26.26 |
| ATOM | 876 | N | GLY | 116 | −8.569 | 26.582 | 42.496 | 1.00 | 25.06 |
| ATOM | 877 | CA | GLY | 116 | −9.889 | 26.767 | 43.066 | 1.00 | 25.41 |
| ATOM | 878 | C | GLY | 116 | −10.964 | 25.864 | 42.490 | 1.00 | 26.85 |
| ATOM | 879 | O | GLY | 116 | −10.767 | 24.659 | 42.346 | 1.00 | 26.53 |
| ATOM | 880 | N | GLU | 117 | −12.105 | 26.468 | 42.166 | 1.00 | 26.32 |
| ATOM | 881 | CA | GLU | 117 | −13.268 | 25.782 | 41.606 | 1.00 | 27.23 |
| ATOM | 882 | CB | GLU | 117 | −14.205 | 26.810 | 40.959 | 1.00 | 31.55 |
| ATOM | 883 | CG | GLU | 117 | −15.191 | 27.444 | 41.923 | 1.00 | 36.76 |
| ATOM | 884 | CD | GLU | 117 | −16.448 | 26.615 | 42.091 | 1.00 | 40.05 |
| ATOM | 885 | OE1 | GLU | 117 | −16.341 | 25.369 | 42.112 | 1.00 | 39.80 |
| ATOM | 886 | OE2 | GLU | 117 | −17.543 | 27.210 | 42.211 | 1.00 | 41.02 |
| ATOM | 887 | C | GLU | 117 | −13.021 | 24.661 | 40.602 | 1.00 | 24.91 |
| ATOM | 888 | O | GLU | 117 | −13.621 | 23.592 | 40.715 | 1.00 | 24.23 |
| ATOM | 889 | N | TRP | 118 | −12.155 | 24.904 | 39.622 | 1.00 | 23.95 |
| ATOM | 890 | CA | TRP | 118 | −11.880 | 23.906 | 38.588 | 1.00 | 23.05 |
| ATOM | 891 | CB | TRP | 118 | −10.853 | 24.437 | 37.574 | 1.00 | 22.93 |
| ATOM | 892 | CG | TRP | 118 | −9.417 | 24.433 | 38.027 | 1.00 | 22.06 |
| ATOM | 893 | CD2 | TRP | 118 | −8.430 | 23.432 | 37.736 | 1.00 | 22.29 |
| ATOM | 894 | CE2 | TRP | 118 | −7.227 | 23.836 | 38.356 | 1.00 | 21.75 |
| ATOM | 895 | CE3 | TRP | 118 | −8.449 | 22.231 | 37.015 | 1.00 | 21.59 |
| ATOM | 896 | CD1 | TRP | 118 | −8.787 | 25.377 | 38.790 | 1.00 | 22.77 |
| ATOM | 897 | NE1 | TRP | 118 | −7.472 | 25.027 | 38.989 | 1.00 | 23.97 |
| ATOM | 898 | CZ2 | TRP | 118 | −6.049 | 23.082 | 38.272 | 1.00 | 21.19 |
| ATOM | 899 | CZ3 | TRP | 118 | −7.273 | 21.478 | 36.932 | 1.00 | 19.76 |
| ATOM | 900 | CH2 | TRP | 118 | −6.093 | 21.908 | 37.558 | 1.00 | 18.03 |
| ATOM | 901 | C | TRP | 118 | −11.415 | 22.562 | 39.137 | 1.00 | 22.73 |
| ATOM | 902 | O | TRP | 118 | −11.504 | 21.542 | 38.453 | 1.00 | 22.61 |
| ATOM | 903 | N | LEU | 119 | −10.933 | 22.563 | 40.375 | 1.00 | 22.53 |
| ATOM | 904 | CA | LEU | 119 | −10.445 | 21.343 | 41.017 | 1.00 | 21.12 |
| ATOM | 905 | CB | LEU | 119 | −9.323 | 21.686 | 42.000 | 1.00 | 22.00 |
| ATOM | 906 | CG | LEU | 119 | −7.951 | 21.956 | 41.397 | 1.00 | 22.79 |
| ATOM | 907 | CD1 | LEU | 119 | −6.976 | 22.370 | 42.489 | 1.00 | 23.33 |
| ATOM | 908 | CD2 | LEU | 119 | −7.469 | 20.690 | 40.691 | 1.00 | 24.30 |
| ATOM | 909 | C | LEU | 119 | −11.502 | 20.541 | 41.759 | 1.00 | 20.72 |
| ATOM | 910 | O | LEU | 119 | −11.232 | 19.422 | 42.193 | 1.00 | 21.92 |
| ATOM | 911 | N | VAL | 120 | −12.699 | 21.101 | 41.908 | 1.00 | 19.20 |
| ATOM | 912 | CA | VAL | 120 | −13.766 | 20.419 | 42.638 | 1.00 | 19.95 |
| ATOM | 913 | CB | VAL | 120 | −15.127 | 21.122 | 42.429 | 1.00 | 22.03 |
| ATOM | 914 | CG1 | VAL | 120 | −16.258 | 20.225 | 42.907 | 1.00 | 24.11 |
| ATOM | 915 | CG2 | VAL | 120 | −15.150 | 22.429 | 43.201 | 1.00 | 25.29 |
| ATOM | 916 | C | VAL | 120 | −13.921 | 18.940 | 42.301 | 1.00 | 18.47 |
| ATOM | 917 | O | VAL | 120 | −13.961 | 18.093 | 43.196 | 1.00 | 16.89 |
| ATOM | 918 | N | GLU | 121 | −14.004 | 18.624 | 41.015 | 1.00 | 17.81 |
| ATOM | 919 | CA | GLU | 121 | −14.163 | 17.237 | 40.611 | 1.00 | 17.82 |
| ATOM | 920 | CB | GLU | 121 | −14.344 | 17.150 | 39.094 | 1.00 | 21.67 |
| ATOM | 921 | CG | GLU | 121 | −14.418 | 15.728 | 38.576 | 1.00 | 25.89 |
| ATOM | 922 | CD | GLU | 121 | −14.824 | 15.658 | 37.114 | 1.00 | 31.46 |
| ATOM | 923 | OE1 | GLU | 121 | −14.246 | 16.399 | 36.290 | 1.00 | 33.28 |
| ATOM | 924 | OE2 | GLU | 121 | −15.717 | 14.848 | 36.793 | 1.00 | 33.12 |
| ATOM | 925 | C | GLU | 121 | −12.977 | 16.385 | 41.057 | 1.00 | 16.63 |
| ATOM | 926 | O | GLU | 121 | −13.153 | 15.288 | 41.592 | 1.00 | 15.69 |
| ATOM | 927 | N | THR | 122 | −11.772 | 16.901 | 40.846 | 1.00 | 16.55 |
| ATOM | 928 | CA | THR | 122 | −10.557 | 16.188 | 41.230 | 1.00 | 15.50 |
| ATOM | 929 | CB | THR | 122 | −9.291 | 17.010 | 40.858 | 1.00 | 16.20 |
| ATOM | 930 | OG1 | THR | 122 | −9.292 | 17.251 | 39.447 | 1.00 | 18.56 |
| ATOM | 931 | CG2 | THR | 122 | −8.009 | 16.250 | 41.215 | 1.00 | 15.78 |
| ATOM | 932 | C | THR | 122 | −10.571 | 15.891 | 42.729 | 1.00 | 15.85 |
| ATOM | 933 | O | THR | 122 | −10.300 | 14.769 | 43.145 | 1.00 | 14.26 |
| ATOM | 934 | N | VAL | 123 | −10.905 | 16.890 | 43.539 | 1.00 | 16.07 |
| ATOM | 935 | CA | VAL | 123 | −10.942 | 16.691 | 44.982 | 1.00 | 15.30 |
| ATOM | 936 | CB | VAL | 123 | −11.265 | 17.993 | 45.722 | 1.00 | 16.32 |
| ATOM | 937 | CG1 | VAL | 123 | −11.382 | 17.733 | 47.217 | 1.00 | 15.84 |
| ATOM | 938 | CG2 | VAL | 123 | −10.194 | 19.023 | 45.431 | 1.00 | 17.06 |
| ATOM | 939 | C | VAL | 123 | −11.964 | 15.649 | 45.392 | 1.00 | 16.79 |
| ATOM | 940 | O | VAL | 123 | −11.685 | 14.793 | 46.237 | 1.00 | 13.71 |
| ATOM | 941 | N | GLN | 124 | −13.155 | 15.728 | 44.803 | 1.00 | 15.90 |
| ATOM | 942 | CA | GLN | 124 | −14.227 | 14.790 | 45.122 | 1.00 | 17.82 |
| ATOM | 943 | CB | GLN | 124 | −15.498 | 15.146 | 44.333 | 1.00 | 19.74 |
| ATOM | 944 | CG | GLN | 124 | −16.018 | 16.566 | 44.586 | 1.00 | 27.73 |
| ATOM | 945 | CD | GLN | 124 | −17.335 | 16.871 | 43.871 | 1.00 | 30.08 |
| ATOM | 946 | OE1 | GLN | 124 | −17.414 | 16.825 | 42.641 | 1.00 | 33.03 |
| ATOM | 947 | NE2 | GLN | 124 | −18.370 | 17.190 | 44.643 | 1.00 | 28.31 |
| ATOM | 948 | C | GLN | 124 | −13.820 | 13.350 | 44.815 | 1.00 | 18.29 |
| ATOM | 949 | O | GLN | 124 | −14.045 | 12.439 | 45.621 | 1.00 | 17.54 |
| ATOM | 950 | N | MET | 125 | −13.218 | 13.151 | 43.648 | 1.00 | 16.58 |
| ATOM | 951 | CA | MET | 125 | −12.798 | 11.820 | 43.239 | 1.00 | 17.11 |
| ATOM | 952 | CB | MET | 125 | −12.553 | 11.792 | 41.725 | 1.00 | 17.13 |
| ATOM | 953 | CG | MET | 125 | −13.843 | 12.007 | 40.930 | 1.00 | 20.76 |
| ATOM | 954 | SD | MET | 125 | −13.598 | 12.024 | 39.156 | 1.00 | 21.82 |
| ATOM | 955 | CE | MET | 125 | −13.472 | 10.274 | 38.825 | 1.00 | 24.59 |
| ATOM | 956 | C | MET | 125 | −11.578 | 11.317 | 44.011 | 1.00 | 15.45 |
| ATOM | 957 | O | MET | 125 | −11.513 | 10.143 | 44.357 | 1.00 | 15.81 |
| ATOM | 958 | N | LEU | 126 | −10.618 | 12.192 | 44.283 | 1.00 | 15.52 |
| ATOM | 959 | CA | LEU | 126 | −9.458 | 11.776 | 45.047 | 1.00 | 16.42 |
| ATOM | 960 | CB | LEU | 126 | −8.486 | 12.941 | 45.215 | 1.00 | 15.46 |
| ATOM | 961 | CG | LEU | 126 | −7.491 | 13.096 | 44.056 | 1.00 | 16.54 |
| ATOM | 962 | CD1 | LEU | 126 | −6.769 | 14.429 | 44.166 | 1.00 | 17.29 |
| ATOM | 963 | CD2 | LEU | 126 | −6.498 | 11.933 | 44.087 | 1.00 | 16.74 |
| ATOM | 964 | C | LEU | 126 | −9.914 | 11.264 | 46.416 | 1.00 | 17.65 |
| ATOM | 965 | O | LEU | 126 | −9.459 | 10.217 | 46.882 | 1.00 | 15.07 |
| ATOM | 966 | N | THR | 127 | −10.831 | 12.001 | 47.043 | 1.00 | 18.88 |
| ATOM | 967 | CA | THR | 127 | −11.370 | 11.648 | 48.357 | 1.00 | 22.38 |
| ATOM | 968 | CB | THR | 127 | −12.489 | 12.630 | 48.788 | 1.00 | 24.31 |
| ATOM | 969 | OG1 | THR | 127 | −11.971 | 13.967 | 48.825 | 1.00 | 30.18 |
| ATOM | 970 | CG2 | THR | 127 | −13.018 | 12.265 | 50.167 | 1.00 | 28.58 |
| ATOM | 971 | C | THR | 127 | −11.950 | 10.235 | 48.406 | 1.00 | 21.91 |
| ATOM | 972 | O | THR | 127 | −11.594 | 9.439 | 49.279 | 1.00 | 20.97 |
| ATOM | 973 | N | GLU | 128 | −12.854 | 9.920 | 47.482 | 1.00 | 23.67 |
| ATOM | 974 | CA | GLU | 128 | −13.455 | 8.589 | 47.473 | 1.00 | 22.51 |
| ATOM | 975 | CB | GLU | 128 | −14.643 | 8.532 | 46.499 | 1.00 | 26.49 |
| ATOM | 976 | CG | GLU | 128 | −14.656 | 9.613 | 45.450 | 1.00 | 27.76 |
| ATOM | 977 | CD | GLU | 128 | −15.846 | 9.512 | 44.501 | 1.00 | 26.90 |
| ATOM | 978 | OE1 | GLU | 128 | −17.002 | 9.469 | 44.969 | 1.00 | 26.96 |
| ATOM | 979 | OE2 | GLU | 128 | −15.625 | 9.488 | 43.281 | 1.00 | 26.94 |
| ATOM | 980 | C | GLU | 128 | −12.435 | 7.505 | 47.140 | 1.00 | 22.91 |
| ATOM | 981 | O | GLU | 128 | −12.641 | 6.333 | 47.458 | 1.00 | 22.08 |
| ATOM | 982 | N | ARG | 129 | −11.324 | 7.895 | 46.520 | 1.00 | 19.89 |
| ATOM | 983 | CA | ARG | 129 | −10.291 | 6.929 | 46.176 | 1.00 | 19.15 |
| ATOM | 984 | CB | ARG | 129 | −9.713 | 7.256 | 44.792 | 1.00 | 18.25 |
| ATOM | 985 | CG | ARG | 129 | −10.712 | 6.934 | 43.664 | 1.00 | 18.33 |
| ATOM | 986 | CD | ARG | 129 | −10.483 | 7.728 | 42.382 | 1.00 | 16.74 |
| ATOM | 987 | NE | ARG | 129 | −11.514 | 7.426 | 41.386 | 1.00 | 14.27 |
| ATOM | 988 | CZ | ARG | 129 | −12.793 | 7.779 | 41.491 | 1.00 | 16.92 |
| ATOM | 989 | NH1 | ARG | 129 | −13.220 | 8.459 | 42.547 | 1.00 | 14.64 |
| ATOM | 990 | NH2 | ARG | 129 | −13.654 | 7.440 | 40.544 | 1.00 | 15.04 |
| ATOM | 991 | C | ARG | 129 | −9.202 | 6.856 | 47.260 | 1.00 | 19.41 |
| ATOM | 992 | O | ARG | 129 | −8.045 | 6.521 | 46.987 | 1.00 | 18.30 |
| ATOM | 993 | N | ALA | 130 | −9.616 | 7.174 | 48.488 | 1.00 | 19.07 |
| ATOM | 994 | CA | ALA | 130 | −8.789 | 7.129 | 49.697 | 1.00 | 17.95 |
| ATOM | 995 | CB | ALA | 130 | −8.201 | 5.722 | 49.868 | 1.00 | 16.84 |
| ATOM | 996 | C | ALA | 130 | −7.674 | 8.164 | 49.860 | 1.00 | 18.35 |
| ATOM | 997 | O | ALA | 130 | −6.821 | 8.015 | 50.738 | 1.00 | 17.72 |
| ATOM | 998 | N | VAL | 131 | −7.684 | 9.211 | 49.043 | 1.00 | 18.16 |
| ATOM | 999 | CA | VAL | 131 | −6.656 | 10.236 | 49.123 | 1.00 | 18.21 |
| ATOM | 1000 | CB | VAL | 131 | −6.059 | 10.538 | 47.733 | 1.00 | 18.83 |
| ATOM | 1001 | CG1 | VAL | 131 | −4.972 | 11.599 | 47.856 | 1.00 | 19.02 |
| ATOM | 1002 | CG2 | VAL | 131 | −5.502 | 9.264 | 47.124 | 1.00 | 18.78 |
| ATOM | 1003 | C | VAL | 131 | −7.162 | 11.550 | 49.704 | 1.00 | 17.21 |
| ATOM | 1004 | O | VAL | 131 | −7.921 | 12.274 | 49.059 | 1.00 | 17.48 |
| ATOM | 1005 | N | PRO | 132 | −6.768 | 11.858 | 50.947 | 1.00 | 17.96 |
| ATOM | 1006 | CD | PRO | 132 | −6.078 | 10.981 | 51.909 | 1.00 | 16.97 |
| ATOM | 1007 | CA | PRO | 132 | −7.195 | 13.109 | 51.580 | 1.00 | 16.82 |
| ATOM | 1008 | CB | PRO | 132 | −6.775 | 12.929 | 53.043 | 1.00 | 17.66 |
| ATOM | 1009 | CG | PRO | 132 | −5.667 | 11.944 | 52.979 | 1.00 | 22.79 |
| ATOM | 1010 | C | PRO | 132 | −6.495 | 14.270 | 50.880 | 1.00 | 16.36 |
| ATOM | 1011 | O | PRO | 132 | −5.335 | 14.159 | 50.465 | 1.00 | 14.89 |
| ATOM | 1012 | N | VAL | 133 | −7.207 | 15.381 | 50.745 | 1.00 | 15.39 |
| ATOM | 1013 | CA | VAL | 133 | −6.666 | 16.534 | 50.046 | 1.00 | 14.12 |
| ATOM | 1014 | CB | VAL | 133 | −7.488 | 16.816 | 48.772 | 1.00 | 14.52 |
| ATOM | 1015 | CG1 | VAL | 133 | −6.921 | 18.016 | 48.037 | 1.00 | 12.24 |
| ATOM | 1016 | CG2 | VAL | 133 | −7.495 | 15.576 | 47.874 | 1.00 | 13.02 |
| ATOM | 1017 | C | VAL | 133 | −6.621 | 17.821 | 50.855 | 1.00 | 14.73 |
| ATOM | 1018 | O | VAL | 133 | −7.526 | 18.116 | 51.632 | 1.00 | 14.95 |
| ATOM | 1019 | N | CYS | 134 | −5.546 | 18.571 | 50.650 | 1.00 | 15.49 |
| ATOM | 1020 | CA | CYS | 134 | −5.355 | 19.868 | 51.273 | 1.00 | 14.56 |
| ATOM | 1021 | CB | CYS | 134 | −4.001 | 19.946 | 51.985 | 1.00 | 14.83 |
| ATOM | 1022 | SG | CYS | 134 | −3.649 | 21.586 | 52.681 | 1.00 | 17.18 |
| ATOM | 1023 | C | CYS | 134 | −5.388 | 20.866 | 50.121 | 1.00 | 16.54 |
| ATOM | 1024 | O | CYS | 134 | −4.642 | 20.734 | 49.143 | 1.00 | 16.64 |
| ATOM | 1025 | N | GLY | 135 | −6.276 | 21.851 | 50.215 | 1.00 | 16.38 |
| ATOM | 1026 | CA | GLY | 135 | −6.371 | 22.864 | 49.176 | 1.00 | 16.44 |
| ATOM | 1027 | C | GLY | 135 | −5.297 | 23.919 | 49.363 | 1.00 | 18.40 |
| ATOM | 1028 | O | GLY | 135 | −4.615 | 23.932 | 50.387 | 1.00 | 17.99 |
| ATOM | 1029 | N | HIS | 136 | −5.150 | 24.806 | 48.382 | 1.00 | 18.50 |
| ATOM | 1030 | CA | HIS | 136 | −4.147 | 25.868 | 48.435 | 1.00 | 18.97 |
| ATOM | 1031 | C | HIS | 136 | −4.624 | 27.055 | 47.603 | 1.00 | 21.58 |
| ATOM | 1032 | O | HIS | 136 | −4.727 | 26.965 | 46.380 | 1.00 | 21.19 |
| ATOM | 1033 | CB | HIS | 136 | −2.810 | 25.332 | 47.899 | 1.00 | 19.18 |
| ATOM | 1034 | CG | HIS | 136 | −1.638 | 26.240 | 48.126 | 1.00 | 19.85 |
| ATOM | 1035 | ND1 | HIS | 136 | −0.349 | 25.774 | 48.029 | 1.00 | 21.61 |
| ATOM | 1036 | CE1 | HIS | 136 | 0.428 | 26.819 | 48.243 | 1.00 | 22.89 |
| ATOM | 1037 | CD2 | HIS | 136 | −1.614 | 27.568 | 48.403 | 1.00 | 19.60 |
| ATOM | 1038 | NE2 | HIS | 136 | −0.291 | 27.929 | 48.474 | 1.00 | 20.06 |
| ATOM | 1039 | N | LEU | 137 | −4.925 | 28.162 | 48.278 | 1.00 | 23.56 |
| ATOM | 1040 | CA | LEU | 137 | −5.399 | 29.374 | 47.617 | 1.00 | 24.34 |
| ATOM | 1041 | CB | LEU | 137 | −6.850 | 29.655 | 48.019 | 1.00 | 25.33 |
| ATOM | 1042 | CG | LEU | 137 | −7.884 | 28.574 | 47.685 | 1.00 | 24.22 |
| ATOM | 1043 | CD1 | LEU | 137 | −9.203 | 28.871 | 48.386 | 1.00 | 24.72 |
| ATOM | 1044 | CD2 | LEU | 137 | −8.072 | 28.510 | 46.181 | 1.00 | 24.79 |
| ATOM | 1045 | C | LEU | 137 | −4.528 | 30.578 | 47.979 | 1.00 | 26.25 |
| ATOM | 1046 | O | LEU | 137 | −3.798 | 30.552 | 48.969 | 1.00 | 25.52 |
| ATOM | 1047 | N | GLY | 138 | −4.617 | 31.630 | 47.169 | 1.00 | 28.18 |
| ATOM | 1048 | CA | GLY | 138 | −3.837 | 32.827 | 47.411 | 1.00 | 30.33 |
| ATOM | 1049 | C | GLY | 138 | −2.702 | 32.926 | 46.414 | 1.00 | 32.93 |
| ATOM | 1050 | O | GLY | 138 | −2.919 | 32.829 | 45.208 | 1.00 | 33.34 |
| ATOM | 1051 | N | LEU | 139 | −1.489 | 33.109 | 46.916 | 1.00 | 34.19 |
| ATOM | 1052 | CA | LEU | 139 | −0.320 | 33.212 | 46.058 | 1.00 | 36.17 |
| ATOM | 1053 | CB | LEU | 139 | 0.784 | 33.982 | 46.787 | 1.00 | 37.11 |
| ATOM | 1054 | CG | LEU | 139 | 1.968 | 34.522 | 45.977 | 1.00 | 39.03 |
| ATOM | 1055 | CD1 | LEU | 139 | 2.838 | 35.383 | 46.884 | 1.00 | 40.33 |
| ATOM | 1056 | CD2 | LEU | 139 | 2.780 | 33.385 | 45.383 | 1.00 | 37.65 |
| ATOM | 1057 | C | LEU | 139 | 0.157 | 31.803 | 45.698 | 1.00 | 37.33 |
| ATOM | 1058 | O | LEU | 139 | 0.914 | 31.184 | 46.442 | 1.00 | 37.46 |
| ATOM | 1059 | N | THR | 140 | −0.304 | 31.303 | 44.555 | 1.00 | 37.52 |
| ATOM | 1060 | CA | THR | 140 | 0.064 | 29.974 | 44.081 | 1.00 | 38.56 |
| ATOM | 1061 | CB | THR | 140 | −1.057 | 29.367 | 43.214 | 1.00 | 39.12 |
| ATOM | 1062 | OG1 | THR | 140 | −1.375 | 30.266 | 42.145 | 1.00 | 38.58 |
| ATOM | 1063 | CG2 | THR | 140 | −2.302 | 29.118 | 44.053 | 1.00 | 38.94 |
| ATOM | 1064 | C | THR | 140 | 1.343 | 30.044 | 43.249 | 1.00 | 38.97 |
| ATOM | 1065 | O | THR | 140 | 1.314 | 30.444 | 42.085 | 1.00 | 39.39 |
| ATOM | 1066 | N | PRO | 141 | 2.483 | 29.646 | 43.837 | 1.00 | 38.90 |
| ATOM | 1067 | CD | PRO | 141 | 2.619 | 29.077 | 45.190 | 1.00 | 39.41 |
| ATOM | 1068 | CA | PRO | 141 | 3.778 | 29.669 | 43.147 | 1.00 | 37.89 |
| ATOM | 1069 | CB | PRO | 141 | 4.742 | 29.115 | 44.197 | 1.00 | 38.82 |
| ATOM | 1070 | CG | PRO | 141 | 3.864 | 28.250 | 45.055 | 1.00 | 40.43 |
| ATOM | 1071 | C | PRO | 141 | 3.831 | 28.899 | 41.831 | 1.00 | 37.49 |
| ATOM | 1072 | O | PRO | 141 | 4.622 | 29.233 | 40.947 | 1.00 | 34.97 |
| ATOM | 1073 | N | GLN | 142 | 2.997 | 27.872 | 41.700 | 1.00 | 35.93 |
| ATOM | 1074 | CA | GLN | 142 | 2.975 | 27.084 | 40.470 | 1.00 | 37.63 |
| ATOM | 1075 | CB | GLN | 142 | 2.072 | 25.855 | 40.635 | 1.00 | 35.67 |
| ATOM | 1076 | CG | GLN | 142 | 2.807 | 24.600 | 41.091 | 1.00 | 34.13 |
| ATOM | 1077 | CD | GLN | 142 | 1.860 | 23.471 | 41.458 | 1.00 | 33.64 |
| ATOM | 1078 | OE1 | GLN | 142 | 0.906 | 23.186 | 40.737 | 1.00 | 31.98 |
| ATOM | 1079 | NE2 | GLN | 142 | 2.127 | 22.817 | 42.583 | 1.00 | 33.66 |
| ATOM | 1080 | C | GLN | 142 | 2.508 | 27.920 | 39.282 | 1.00 | 38.52 |
| ATOM | 1081 | O | GLN | 142 | 2.859 | 27.635 | 38.134 | 1.00 | 38.49 |
| ATOM | 1082 | N | SER | 143 | 1.718 | 28.953 | 39.562 | 1.00 | 39.73 |
| ATOM | 1083 | CA | SER | 143 | 1.210 | 29.834 | 38.513 | 1.00 | 40.92 |
| ATOM | 1084 | CB | SER | 143 | −0.279 | 30.123 | 38.732 | 1.00 | 40.43 |
| ATOM | 1085 | OG | SER | 143 | −1.059 | 28.946 | 38.606 | 1.00 | 41.04 |
| ATOM | 1086 | C | SER | 143 | 1.982 | 31.149 | 38.484 | 1.00 | 41.64 |
| ATOM | 1087 | O | SER | 143 | 1.448 | 32.178 | 38.076 | 1.00 | 42.46 |
| ATOM | 1088 | N | VAL | 144 | 3.238 | 31.110 | 38.916 | 1.00 | 42.62 |
| ATOM | 1089 | CA | VAL | 144 | 4.075 | 32.306 | 38.935 | 1.00 | 43.78 |
| ATOM | 1090 | CB | VAL | 144 | 5.483 | 31.995 | 39.492 | 1.00 | 43.30 |
| ATOM | 1091 | CG1 | VAL | 144 | 6.194 | 31.001 | 38.588 | 1.00 | 43.74 |
| ATOM | 1092 | CG2 | VAL | 144 | 6.291 | 33.278 | 39.616 | 1.00 | 44.06 |
| ATOM | 1093 | C | VAL | 144 | 4.219 | 32.918 | 37.541 | 1.00 | 44.58 |
| ATOM | 1094 | O | VAL | 144 | 4.423 | 34.123 | 37.405 | 1.00 | 44.39 |
| ATOM | 1095 | N | ASN | 145 | 4.111 | 32.082 | 36.512 | 1.00 | 45.34 |
| ATOM | 1096 | CA | ASN | 145 | 4.231 | 32.540 | 35.130 | 1.00 | 47.01 |
| ATOM | 1097 | CB | ASN | 145 | 4.606 | 31.368 | 34.214 | 1.00 | 47.34 |
| ATOM | 1098 | CG | ASN | 145 | 5.992 | 30.821 | 34.505 | 1.00 | 48.01 |
| ATOM | 1099 | OD1 | ASN | 145 | 7.000 | 31.488 | 34.264 | 1.00 | 47.54 |
| ATOM | 1100 | ND2 | ASN | 145 | 6.049 | 29.602 | 35.031 | 1.00 | 48.81 |
| ATOM | 1101 | C | ASN | 145 | 2.938 | 33.179 | 34.640 | 1.00 | 47.35 |
| ATOM | 1102 | O | ASN | 145 | 2.905 | 33.813 | 33.585 | 1.00 | 47.69 |
| ATOM | 1103 | N | ILE | 146 | 1.872 | 33.005 | 35.410 | 1.00 | 48.31 |
| ATOM | 1104 | CA | ILE | 146 | 0.578 | 33.569 | 35.055 | 1.00 | 49.69 |
| ATOM | 1105 | CB | ILE | 146 | −0.574 | 32.693 | 35.589 | 1.00 | 49.13 |
| ATOM | 1106 | CG2 | ILE | 146 | −1.912 | 33.335 | 35.263 | 1.00 | 48.60 |
| ATOM | 1107 | CG1 | ILE | 146 | −0.485 | 31.290 | 34.981 | 1.00 | 48.60 |
| ATOM | 1108 | CD1 | ILE | 146 | −0.628 | 31.262 | 33.472 | 1.00 | 48.16 |
| ATOM | 1109 | C | ILE | 146 | 0.426 | 34.976 | 35.623 | 1.00 | 51.32 |
| ATOM | 1110 | O | ILE | 146 | −0.045 | 35.880 | 34.934 | 1.00 | 51.26 |
| ATOM | 1111 | N | PHE | 147 | 0.831 | 35.154 | 36.879 | 1.00 | 52.78 |
| ATOM | 1112 | CA | PHE | 147 | 0.738 | 36.448 | 37.553 | 1.00 | 54.17 |
| ATOM | 1113 | CB | PHE | 147 | 0.713 | 36.254 | 39.072 | 1.00 | 55.12 |
| ATOM | 1114 | CG | PHE | 147 | −0.198 | 35.152 | 39.528 | 1.00 | 56.36 |
| ATOM | 1115 | CD1 | PHE | 147 | −1.553 | 35.177 | 39.218 | 1.00 | 57.26 |
| ATOM | 1116 | CD2 | PHE | 147 | 0.303 | 34.084 | 40.266 | 1.00 | 57.27 |
| ATOM | 1117 | CE1 | PHE | 147 | −2.400 | 34.151 | 39.634 | 1.00 | 57.90 |
| ATOM | 1118 | CE2 | PHE | 147 | −0.533 | 33.052 | 40.688 | 1.00 | 57.81 |
| ATOM | 1119 | CZ | PHE | 147 | −1.887 | 33.086 | 40.371 | 1.00 | 58.12 |
| ATOM | 1120 | C | PHE | 147 | 1.914 | 37.350 | 37.188 | 1.00 | 54.46 |
| ATOM | 1121 | O | PHE | 147 | 1.937 | 38.528 | 37.544 | 1.00 | 54.14 |
| ATOM | 1122 | N | GLY | 148 | 2.890 | 36.787 | 36.483 | 1.00 | 55.28 |
| ATOM | 1123 | CA | GLY | 148 | 4.056 | 37.555 | 36.090 | 1.00 | 56.16 |
| ATOM | 1124 | C | GLY | 148 | 4.972 | 37.817 | 37.268 | 1.00 | 56.63 |
| ATOM | 1125 | O | GLY | 148 | 5.699 | 38.809 | 37.294 | 1.00 | 56.49 |
| ATOM | 1126 | N | GLY | 149 | 4.933 | 36.920 | 38.249 | 1.00 | 57.50 |
| ATOM | 1127 | CA | GLY | 149 | 5.761 | 37.068 | 39.431 | 1.00 | 58.67 |
| ATOM | 1128 | C | GLY | 149 | 4.997 | 36.690 | 40.685 | 1.00 | 59.94 |
| ATOM | 1129 | O | GLY | 149 | 3.828 | 36.305 | 40.613 | 1.00 | 59.46 |
| ATOM | 1130 | N | TYR | 150 | 5.653 | 36.797 | 41.837 | 1.00 | 61.00 |
| ATOM | 1131 | CA | TYR | 150 | 5.018 | 36.467 | 43.108 | 1.00 | 62.09 |
| ATOM | 1132 | CB | TYR | 150 | 6.029 | 35.830 | 44.068 | 1.00 | 62.64 |
| ATOM | 1133 | CG | TYR | 150 | 6.779 | 34.653 | 43.484 | 1.00 | 63.79 |
| ATOM | 1134 | CD1 | TYR | 150 | 7.910 | 34.846 | 42.688 | 1.00 | 64.15 |
| ATOM | 1135 | CE1 | TYR | 150 | 8.601 | 33.764 | 42.142 | 1.00 | 64.08 |
| ATOM | 1136 | CD2 | TYR | 150 | 6.355 | 33.344 | 43.718 | 1.00 | 64.03 |
| ATOM | 1137 | CE2 | TYR | 150 | 7.038 | 32.256 | 43.175 | 1.00 | 63.87 |
| ATOM | 1138 | CZ | TYR | 150 | 8.160 | 32.474 | 42.389 | 1.00 | 64.27 |
| ATOM | 1139 | OH | TYR | 150 | 8.840 | 31.407 | 41.849 | 1.00 | 64.43 |
| ATOM | 1140 | C | TYR | 150 | 4.426 | 37.721 | 43.744 | 1.00 | 62.40 |
| ATOM | 1141 | O | TYR | 150 | 5.141 | 38.523 | 44.347 | 1.00 | 62.18 |
| ATOM | 1142 | N | LYS | 151 | 3.113 | 37.883 | 43.603 | 1.00 | 62.64 |
| ATOM | 1143 | CA | LYS | 151 | 2.414 | 39.038 | 44.152 | 1.00 | 62.63 |
| ATOM | 1144 | CB | LYS | 151 | 1.768 | 39.842 | 43.020 | 1.00 | 63.52 |
| ATOM | 1145 | CG | LYS | 151 | 2.765 | 40.359 | 41.993 | 1.00 | 64.61 |
| ATOM | 1146 | CD | LYS | 151 | 2.085 | 41.186 | 40.916 | 1.00 | 65.48 |
| ATOM | 1147 | CE | LYS | 151 | 3.100 | 41.731 | 39.921 | 1.00 | 66.46 |
| ATOM | 1148 | NZ | LYS | 151 | 2.464 | 42.586 | 38.878 | 1.00 | 66.97 |
| ATOM | 1149 | C | LYS | 151 | 1.351 | 38.615 | 45.161 | 1.00 | 62.18 |
| ATOM | 1150 | O | LYS | 151 | 1.022 | 37.433 | 45.273 | 1.00 | 62.06 |
| ATOM | 1151 | N | VAL | 152 | 0.815 | 39.586 | 45.893 | 1.00 | 61.53 |
| ATOM | 1152 | CA | VAL | 152 | −0.204 | 39.309 | 46.897 | 1.00 | 60.97 |
| ATOM | 1153 | CB | VAL | 152 | −0.190 | 40.378 | 48.010 | 1.00 | 60.76 |
| ATOM | 1154 | CG1 | VAL | 152 | −1.225 | 40.039 | 49.073 | 1.00 | 60.74 |
| ATOM | 1155 | CG2 | VAL | 152 | 1.195 | 40.468 | 48.624 | 1.00 | 60.69 |
| ATOM | 1156 | C | VAL | 152 | −1.601 | 39.263 | 46.293 | 1.00 | 60.64 |
| ATOM | 1157 | O | VAL | 152 | −1.999 | 40.166 | 45.559 | 1.00 | 60.43 |
| ATOM | 1158 | N | GLN | 153 | −2.341 | 38.205 | 46.608 | 1.00 | 60.42 |
| ATOM | 1159 | CA | GLN | 153 | −3.703 | 38.042 | 46.111 | 1.00 | 60.14 |
| ATOM | 1160 | CB | GLN | 153 | −3.918 | 36.620 | 45.581 | 1.00 | 60.93 |
| ATOM | 1161 | CG | GLN | 153 | −3.467 | 36.402 | 44.140 | 1.00 | 61.33 |
| ATOM | 1162 | CD | GLN | 153 | −1.977 | 36.593 | 43.953 | 1.00 | 61.41 |
| ATOM | 1163 | OE1 | GLN | 153 | −1.169 | 35.901 | 44.572 | 1.00 | 62.43 |
| ATOM | 1164 | NE2 | GLN | 153 | −1.605 | 37.531 | 43.091 | 1.00 | 60.96 |
| ATOM | 1165 | C | GLN | 153 | −4.715 | 38.333 | 47.214 | 1.00 | 59.42 |
| ATOM | 1166 | O | GLN | 153 | −4.350 | 38.483 | 48.379 | 1.00 | 59.06 |
| ATOM | 1167 | N | GLY | 154 | −5.988 | 38.410 | 46.838 | 1.00 | 58.76 |
| ATOM | 1168 | CA | GLY | 154 | −7.030 | 38.685 | 47.808 | 1.00 | 58.28 |
| ATOM | 1169 | C | GLY | 154 | −7.425 | 40.149 | 47.825 | 1.00 | 57.80 |
| ATOM | 1170 | O | GLY | 154 | −8.548 | 40.489 | 48.199 | 1.00 | 56.85 |
| ATOM | 1171 | N | ARG | 155 | −6.497 | 41.013 | 47.421 | 1.00 | 58.06 |
| ATOM | 1172 | CA | ARG | 155 | −6.732 | 42.455 | 47.380 | 1.00 | 58.09 |
| ATOM | 1173 | CB | ARG | 155 | −5.535 | 43.174 | 46.742 | 1.00 | 59.37 |
| ATOM | 1174 | CG | ARG | 155 | −4.204 | 42.996 | 47.470 | 1.00 | 60.93 |
| ATOM | 1175 | CD | ARG | 155 | −4.242 | 43.586 | 48.873 | 1.00 | 62.00 |
| ATOM | 1176 | NE | ARG | 155 | −2.977 | 43.409 | 49.587 | 1.00 | 62.99 |
| ATOM | 1177 | CZ | ARG | 155 | −1.826 | 43.969 | 49.227 | 1.00 | 63.25 |
| ATOM | 1178 | NH1 | ARG | 155 | −1.773 | 44.750 | 48.157 | 1.00 | 63.93 |
| ATOM | 1179 | NH2 | ARG | 155 | −0.727 | 43.753 | 49.938 | 1.00 | 63.47 |
| ATOM | 1180 | C | ARG | 155 | −7.990 | 42.771 | 46.575 | 1.00 | 57.05 |
| ATOM | 1181 | O | ARG | 155 | −8.123 | 42.355 | 45.425 | 1.00 | 57.28 |
| ATOM | 1182 | N | GLY | 156 | −8.908 | 43.513 | 47.184 | 1.00 | 55.32 |
| ATOM | 1183 | CA | GLY | 156 | −10.136 | 43.862 | 46.498 | 1.00 | 53.27 |
| ATOM | 1184 | C | GLY | 156 | −11.306 | 43.028 | 46.976 | 1.00 | 52.09 |
| ATOM | 1185 | O | GLY | 156 | −11.123 | 41.930 | 47.501 | 1.00 | 51.92 |
| ATOM | 1186 | N | ASP | 157 | −12.511 | 43.553 | 46.790 | 1.00 | 50.40 |
| ATOM | 1187 | CA | ASP | 157 | −13.731 | 42.872 | 47.208 | 1.00 | 49.29 |
| ATOM | 1188 | CB | ASP | 157 | −14.914 | 43.832 | 47.090 | 1.00 | 50.74 |
| ATOM | 1189 | CG | ASP | 157 | −14.693 | 45.121 | 47.854 | 1.00 | 51.46 |
| ATOM | 1190 | OD1 | ASP | 157 | −14.893 | 45.122 | 49.086 | 1.00 | 50.41 |
| ATOM | 1191 | OD2 | ASP | 157 | −14.304 | 46.126 | 47.216 | 1.00 | 53.02 |
| ATOM | 1192 | C | ASP | 157 | −13.991 | 41.634 | 46.358 | 1.00 | 48.08 |
| ATOM | 1193 | O | ASP | 157 | −14.231 | 40.544 | 46.881 | 1.00 | 46.48 |
| ATOM | 1194 | N | GLU | 158 | −13.942 | 41.813 | 45.043 | 1.00 | 46.08 |
| ATOM | 1195 | CA | GLU | 158 | −14.178 | 40.718 | 44.116 | 1.00 | 45.63 |
| ATOM | 1196 | CB | GLU | 158 | −14.092 | 41.225 | 42.675 | 1.00 | 48.09 |
| ATOM | 1197 | CG | GLU | 158 | −14.387 | 40.169 | 41.626 | 1.00 | 50.76 |
| ATOM | 1198 | CD | GLU | 158 | −14.503 | 40.755 | 40.233 | 1.00 | 53.13 |
| ATOM | 1199 | OE1 | GLU | 158 | −15.430 | 41.564 | 40.002 | 1.00 | 55.10 |
| ATOM | 1200 | OE2 | GLU | 158 | −13.670 | 40.411 | 39.367 | 1.00 | 54.38 |
| ATOM | 1201 | C | GLU | 158 | −13.187 | 39.578 | 44.329 | 1.00 | 43.56 |
| ATOM | 1202 | O | GLU | 158 | −13.584 | 38.429 | 44.529 | 1.00 | 42.93 |
| ATOM | 1203 | N | ALA | 159 | −11.898 | 39.899 | 44.287 | 1.00 | 41.57 |
| ATOM | 1204 | CA | ALA | 159 | −10.859 | 38.893 | 44.482 | 1.00 | 39.78 |
| ATOM | 1205 | CB | ALA | 159 | −9.482 | 39.547 | 44.444 | 1.00 | 39.93 |
| ATOM | 1206 | C | ALA | 159 | −11.065 | 38.177 | 45.814 | 1.00 | 38.91 |
| ATOM | 1207 | O | ALA | 159 | −10.917 | 36.958 | 45.904 | 1.00 | 37.78 |
| ATOM | 1208 | N | GLY | 160 | −11.419 | 38.944 | 46.840 | 1.00 | 36.56 |
| ATOM | 1209 | CA | GLY | 160 | −11.642 | 38.371 | 48.152 | 1.00 | 35.21 |
| ATOM | 1210 | C | GLY | 160 | −12.818 | 37.416 | 48.194 | 1.00 | 35.10 |
| ATOM | 1211 | O | GLY | 160 | −12.718 | 36.330 | 48.768 | 1.00 | 34.10 |
| ATOM | 1212 | N | ASP | 161 | −13.935 | 37.813 | 47.591 | 1.00 | 33.83 |
| ATOM | 1213 | CA | ASP | 161 | −15.126 | 36.971 | 47.575 | 1.00 | 33.55 |
| ATOM | 1214 | CB | ASP | 161 | −16.335 | 37.747 | 47.038 | 1.00 | 34.86 |
| ATOM | 1215 | CG | ASP | 161 | −16.651 | 38.986 | 47.861 | 1.00 | 35.97 |
| ATOM | 1216 | OD1 | ASP | 161 | −16.702 | 38.890 | 49.109 | 1.00 | 34.80 |
| ATOM | 1217 | OD2 | ASP | 161 | −16.862 | 40.056 | 47.255 | 1.00 | 37.49 |
| ATOM | 1218 | C | ASP | 161 | −14.897 | 35.727 | 46.718 | 1.00 | 32.66 |
| ATOM | 1219 | O | ASP | 161 | −15.553 | 34.704 | 46.910 | 1.00 | 30.79 |
| ATOM | 1220 | N | GLN | 162 | −13.967 | 35.822 | 45.773 | 1.00 | 33.43 |
| ATOM | 1221 | CA | GLN | 162 | −13.657 | 34.696 | 44.901 | 1.00 | 34.32 |
| ATOM | 1222 | CB | GLN | 162 | −12.810 | 35.160 | 43.712 | 1.00 | 35.93 |
| ATOM | 1223 | CG | GLN | 162 | −12.549 | 34.069 | 42.680 | 1.00 | 40.84 |
| ATOM | 1224 | CD | GLN | 162 | −13.827 | 33.377 | 42.235 | 1.00 | 43.13 |
| ATOM | 1225 | OE1 | GLN | 162 | −14.770 | 34.023 | 41.774 | 1.00 | 45.74 |
| ATOM | 1226 | NE2 | GLN | 162 | −13.866 | 32.056 | 42.374 | 1.00 | 44.65 |
| ATOM | 1227 | C | GLN | 162 | −12.915 | 33.613 | 45.683 | 1.00 | 32.85 |
| ATOM | 1228 | O | GLN | 162 | −13.236 | 32.429 | 45.575 | 1.00 | 32.77 |
| ATOM | 1229 | N | LEU | 163 | −11.928 | 34.022 | 46.474 | 1.00 | 31.71 |
| ATOM | 1230 | CA | LEU | 163 | −11.159 | 33.074 | 47.275 | 1.00 | 31.82 |
| ATOM | 1231 | CB | LEU | 163 | −10.025 | 33.792 | 48.017 | 1.00 | 33.87 |
| ATOM | 1232 | CG | LEU | 163 | −8.879 | 34.358 | 47.173 | 1.00 | 35.14 |
| ATOM | 1233 | CD1 | LEU | 163 | −7.918 | 35.131 | 48.064 | 1.00 | 36.34 |
| ATOM | 1234 | CD2 | LEU | 163 | −8.146 | 33.222 | 46.472 | 1.00 | 36.72 |
| ATOM | 1235 | C | LEU | 163 | −12.059 | 32.363 | 48.279 | 1.00 | 31.54 |
| ATOM | 1236 | O | LEU | 163 | −11.968 | 31.150 | 48.456 | 1.00 | 30.72 |
| ATOM | 1237 | N | LEU | 164 | −12.932 | 33.124 | 48.934 | 1.00 | 30.37 |
| ATOM | 1238 | CA | LEU | 164 | −13.848 | 32.556 | 49.915 | 1.00 | 29.45 |
| ATOM | 1239 | CB | LEU | 164 | −14.702 | 33.669 | 50.530 | 1.00 | 31.08 |
| ATOM | 1240 | CG | LEU | 164 | −15.296 | 33.451 | 51.925 | 1.00 | 31.21 |
| ATOM | 1241 | CD1 | LEU | 164 | −16.045 | 34.715 | 52.346 | 1.00 | 35.31 |
| ATOM | 1242 | CD2 | LEU | 164 | −16.218 | 32.261 | 51.937 | 1.00 | 32.29 |
| ATOM | 1243 | C | LEU | 164 | −14.737 | 31.540 | 49.203 | 1.00 | 28.86 |
| ATOM | 1244 | O | LEU | 164 | −15.058 | 30.480 | 49.744 | 1.00 | 28.39 |
| ATOM | 1245 | N | SER | 165 | −15.126 | 31.876 | 47.978 | 1.00 | 27.45 |
| ATOM | 1246 | CA | SER | 165 | −15.973 | 31.004 | 47.173 | 1.00 | 28.46 |
| ATOM | 1247 | CB | SER | 165 | −16.356 | 31.709 | 45.867 | 1.00 | 26.87 |
| ATOM | 1248 | OG | SER | 165 | −17.280 | 30.932 | 45.133 | 1.00 | 31.92 |
| ATOM | 1249 | C | SER | 165 | −15.243 | 29.699 | 46.856 | 1.00 | 26.43 |
| ATOM | 1250 | O | SER | 165 | −15.796 | 28.611 | 47.010 | 1.00 | 27.06 |
| ATOM | 1251 | N | ASP | 166 | −13.997 | 29.820 | 46.412 | 1.00 | 26.79 |
| ATOM | 1252 | CA | ASP | 166 | −13.194 | 28.648 | 46.078 | 1.00 | 25.84 |
| ATOM | 1253 | CB | ASP | 166 | −11.881 | 29.057 | 45.407 | 1.00 | 27.39 |
| ATOM | 1254 | CG | ASP | 166 | −12.086 | 29.645 | 44.028 | 1.00 | 28.71 |
| ATOM | 1255 | OD1 | ASP | 166 | −12.913 | 29.099 | 43.262 | 1.00 | 31.41 |
| ATOM | 1256 | OD2 | ASP | 166 | −11.407 | 30.644 | 43.696 | 1.00 | 28.72 |
| ATOM | 1257 | C | ASP | 166 | −12.886 | 27.840 | 47.331 | 1.00 | 25.65 |
| ATOM | 1258 | O | ASP | 166 | −12.769 | 26.615 | 47.275 | 1.00 | 25.23 |
| ATOM | 1259 | N | ALA | 167 | −12.750 | 28.534 | 48.459 | 1.00 | 23.48 |
| ATOM | 1260 | CA | ALA | 167 | −12.454 | 27.893 | 49.733 | 1.00 | 22.78 |
| ATOM | 1261 | CB | ALA | 167 | −12.184 | 28.956 | 50.806 | 1.00 | 23.00 |
| ATOM | 1262 | C | ALA | 167 | −13.599 | 26.991 | 50.169 | 1.00 | 21.48 |
| ATOM | 1263 | O | ALA | 167 | −13.387 | 25.833 | 50.532 | 1.00 | 19.60 |
| ATOM | 1264 | N | LEU | 168 | −14.817 | 27.523 | 50.150 | 1.00 | 21.01 |
| ATOM | 1265 | CA | LEU | 168 | −15.983 | 26.734 | 50.534 | 1.00 | 20.93 |
| ATOM | 1266 | CB | LEU | 168 | −17.228 | 27.622 | 50.596 | 1.00 | 21.15 |
| ATOM | 1267 | CG | LEU | 168 | −17.387 | 28.514 | 51.831 | 1.00 | 19.76 |
| ATOM | 1268 | CD1 | LEU | 168 | −18.297 | 29.693 | 51.501 | 1.00 | 21.84 |
| ATOM | 1269 | CD2 | LEU | 168 | −17.967 | 27.699 | 52.978 | 1.00 | 20.27 |
| ATOM | 1270 | C | LEU | 168 | −16.199 | 25.630 | 49.509 | 1.00 | 20.35 |
| ATOM | 1271 | O | LEU | 168 | −16.610 | 24.527 | 49.855 | 1.00 | 21.26 |
| ATOM | 1272 | N | ALA | 169 | −15.925 | 25.943 | 48.248 | 1.00 | 21.84 |
| ATOM | 1273 | CA | ALA | 169 | −16.088 | 24.984 | 47.161 | 1.00 | 22.58 |
| ATOM | 1274 | CB | ALA | 169 | −15.774 | 25.648 | 45.829 | 1.00 | 19.71 |
| ATOM | 1275 | C | ALA | 169 | −15.198 | 23.764 | 47.368 | 1.00 | 23.26 |
| ATOM | 1276 | O | ALA | 169 | −15.638 | 22.626 | 47.192 | 1.00 | 23.84 |
| ATOM | 1277 | N | LEU | 170 | −13.944 | 24.004 | 47.737 | 1.00 | 22.11 |
| ATOM | 1278 | CA | LEU | 170 | −13.005 | 22.915 | 47.974 | 1.00 | 21.77 |
| ATOM | 1279 | CB | LEU | 170 | −11.585 | 23.473 | 48.169 | 1.00 | 19.66 |
| ATOM | 1280 | CG | LEU | 170 | −10.934 | 24.127 | 46.939 | 1.00 | 17.91 |
| ATOM | 1281 | CD1 | LEU | 170 | −9.666 | 24.896 | 47.340 | 1.00 | 19.18 |
| ATOM | 1282 | CD2 | LEU | 170 | −10.595 | 23.043 | 45.910 | 1.00 | 18.15 |
| ATOM | 1283 | C | LEU | 170 | −13.447 | 22.114 | 49.198 | 1.00 | 20.95 |
| ATOM | 1284 | O | LEU | 170 | −13.408 | 20.888 | 49.185 | 1.00 | 20.34 |
| ATOM | 1285 | N | GLU | 171 | −13.883 | 22.803 | 50.253 | 1.00 | 21.68 |
| ATOM | 1286 | CA | GLU | 171 | −14.332 | 22.116 | 51.461 | 1.00 | 21.25 |
| ATOM | 1287 | CB | GLU | 171 | −14.746 | 23.129 | 52.536 | 1.00 | 22.53 |
| ATOM | 1288 | CG | GLU | 171 | −15.385 | 22.502 | 53.775 | 1.00 | 25.91 |
| ATOM | 1289 | CD | GLU | 171 | −15.745 | 23.536 | 54.834 | 1.00 | 27.19 |
| ATOM | 1290 | OE1 | GLU | 171 | −16.404 | 24.538 | 54.477 | 1.00 | 27.15 |
| ATOM | 1291 | OE2 | GLU | 171 | −15.377 | 23.342 | 56.016 | 1.00 | 27.26 |
| ATOM | 1292 | C | GLU | 171 | −15.504 | 21.195 | 51.149 | 1.00 | 21.24 |
| ATOM | 1293 | O | GLU | 171 | −15.538 | 20.044 | 51.592 | 1.00 | 21.38 |
| ATOM | 1294 | N | ALA | 172 | −16.460 | 21.701 | 50.374 | 1.00 | 21.12 |
| ATOM | 1295 | CA | ALA | 172 | −17.637 | 20.919 | 50.013 | 1.00 | 21.23 |
| ATOM | 1296 | CB | ALA | 172 | −18.651 | 21.807 | 49.299 | 1.00 | 21.68 |
| ATOM | 1297 | C | ALA | 172 | −17.272 | 19.724 | 49.134 | 1.00 | 20.21 |
| ATOM | 1298 | O | ALA | 172 | −17.953 | 18.695 | 49.147 | 1.00 | 19.47 |
| ATOM | 1299 | N | ALA | 173 | −16.192 | 19.866 | 48.374 | 1.00 | 22.48 |
| ATOM | 1300 | CA | ALA | 173 | −15.725 | 18.801 | 47.491 | 1.00 | 19.03 |
| ATOM | 1301 | CB | ALA | 173 | −14.767 | 19.379 | 46.444 | 1.00 | 20.74 |
| ATOM | 1302 | C | ALA | 173 | −15.034 | 17.690 | 48.286 | 1.00 | 21.09 |
| ATOM | 1303 | O | ALA | 173 | −14.845 | 16.575 | 47.792 | 1.00 | 20.88 |
| ATOM | 1304 | N | GLY | 174 | −14.651 | 18.000 | 49.520 | 1.00 | 20.56 |
| ATOM | 1305 | CA | GLY | 174 | −14.011 | 16.998 | 50.352 | 1.00 | 20.44 |
| ATOM | 1306 | C | GLY | 174 | −12.664 | 17.365 | 50.949 | 1.00 | 19.70 |
| ATOM | 1307 | O | GLY | 174 | −12.077 | 16.559 | 51.669 | 1.00 | 19.40 |
| ATOM | 1308 | N | ALA | 175 | −12.157 | 18.559 | 50.658 | 1.00 | 20.44 |
| ATOM | 1309 | CA | ALA | 175 | −10.871 | 18.964 | 51.218 | 1.00 | 19.55 |
| ATOM | 1310 | CB | ALA | 175 | −10.464 | 20.316 | 50.677 | 1.00 | 20.28 |
| ATOM | 1311 | C | ALA | 175 | −10.972 | 19.006 | 52.747 | 1.00 | 21.76 |
| ATOM | 1312 | O | ALA | 175 | −11.891 | 19.610 | 53.297 | 1.00 | 20.48 |
| ATOM | 1313 | N | GLN | 176 | −10.037 | 18.347 | 53.431 | 1.00 | 20.03 |
| ATOM | 1314 | CA | GLN | 176 | −10.041 | 18.310 | 54.892 | 1.00 | 20.58 |
| ATOM | 1315 | CB | GLN | 176 | −9.654 | 16.916 | 55.392 | 1.00 | 20.99 |
| ATOM | 1316 | CG | GLN | 176 | −10.582 | 15.828 | 54.889 | 1.00 | 23.71 |
| ATOM | 1317 | CD | GLN | 176 | −10.327 | 14.487 | 55.535 | 1.00 | 25.55 |
| ATOM | 1318 | OE1 | GLN | 176 | −10.614 | 14.291 | 56.713 | 1.00 | 29.79 |
| ATOM | 1319 | NE2 | GLN | 176 | −9.785 | 13.554 | 54.766 | 1.00 | 27.44 |
| ATOM | 1320 | C | GLN | 176 | −9.096 | 19.349 | 55.487 | 1.00 | 20.58 |
| ATOM | 1321 | O | GLN | 176 | −9.021 | 19.515 | 56.705 | 1.00 | 19.49 |
| ATOM | 1322 | N | LEU | 177 | −8.376 | 20.037 | 54.610 | 1.00 | 20.99 |
| ATOM | 1323 | CA | LEU | 177 | −7.440 | 21.074 | 55.001 | 1.00 | 21.65 |
| ATOM | 1324 | CB | LEU | 177 | −6.063 | 20.478 | 55.315 | 1.00 | 22.64 |
| ATOM | 1325 | CG | LEU | 177 | −5.821 | 20.041 | 56.763 | 1.00 | 25.50 |
| ATOM | 1326 | CD1 | LEU | 177 | −4.483 | 19.321 | 56.874 | 1.00 | 24.79 |
| ATOM | 1327 | CD2 | LEU | 177 | −5.838 | 21.272 | 57.670 | 1.00 | 24.33 |
| ATOM | 1328 | C | LEU | 177 | −7.308 | 22.097 | 53.885 | 1.00 | 22.00 |
| ATOM | 1329 | O | LEU | 177 | −7.529 | 21.792 | 52.709 | 1.00 | 20.39 |
| ATOM | 1330 | N | LEU | 178 | −6.948 | 23.317 | 54.261 | 1.00 | 19.35 |
| ATOM | 1331 | CA | LEU | 178 | −6.779 | 24.386 | 53.295 | 1.00 | 20.42 |
| ATOM | 1332 | CB | LEU | 178 | −8.063 | 25.211 | 53.180 | 1.00 | 19.08 |
| ATOM | 1333 | CG | LEU | 178 | −7.947 | 26.457 | 52.297 | 1.00 | 21.83 |
| ATOM | 1334 | CD1 | LEU | 178 | −7.793 | 26.056 | 50.832 | 1.00 | 22.51 |
| ATOM | 1335 | CD2 | LEU | 178 | −9.187 | 27.327 | 52.472 | 1.00 | 21.62 |
| ATOM | 1336 | C | LEU | 178 | −5.630 | 25.304 | 53.681 | 1.00 | 21.17 |
| ATOM | 1337 | O | LEU | 178 | −5.499 | 25.703 | 54.838 | 1.00 | 21.61 |
| ATOM | 1338 | N | VAL | 179 | −4.803 | 25.630 | 52.696 | 1.00 | 20.87 |
| ATOM | 1339 | CA | VAL | 179 | −3.672 | 26.518 | 52.893 | 1.00 | 21.64 |
| ATOM | 1340 | CB | VAL | 179 | −2.360 | 25.910 | 52.320 | 1.00 | 21.83 |
| ATOM | 1341 | CG1 | VAL | 179 | −1.280 | 26.985 | 52.238 | 1.00 | 21.50 |
| ATOM | 1342 | CG2 | VAL | 179 | −1.876 | 24.760 | 53.204 | 1.00 | 16.70 |
| ATOM | 1343 | C | VAL | 179 | −3.945 | 27.843 | 52.182 | 1.00 | 23.89 |
| ATOM | 1344 | O | VAL | 179 | −4.370 | 27.865 | 51.020 | 1.00 | 22.84 |
| ATOM | 1345 | N | LEU | 180 | −3.718 | 28.941 | 52.900 | 1.00 | 25.31 |
| ATOM | 1346 | CA | LEU | 180 | −3.898 | 30.291 | 52.363 | 1.00 | 27.62 |
| ATOM | 1347 | CB | LEU | 180 | −4.873 | 31.098 | 53.223 | 1.00 | 29.45 |
| ATOM | 1348 | CG | LEU | 180 | −6.349 | 30.724 | 53.135 | 1.00 | 32.82 |
| ATOM | 1349 | CD1 | LEU | 180 | −7.138 | 31.542 | 54.147 | 1.00 | 31.62 |
| ATOM | 1350 | CD2 | LEU | 180 | −6.861 | 30.980 | 51.720 | 1.00 | 30.67 |
| ATOM | 1351 | C | LEU | 180 | −2.537 | 30.962 | 52.398 | 1.00 | 27.37 |
| ATOM | 1352 | O | LEU | 180 | −1.943 | 31.094 | 53.464 | 1.00 | 26.56 |
| ATOM | 1353 | N | GLU | 181 | −2.052 | 31.383 | 51.235 | 1.00 | 28.04 |
| ATOM | 1354 | CA | GLU | 181 | −0.743 | 32.017 | 51.131 | 1.00 | 30.09 |
| ATOM | 1355 | CB | GLU | 181 | 0.131 | 31.261 | 50.123 | 1.00 | 30.40 |
| ATOM | 1356 | CG | GLU | 181 | 1.579 | 31.735 | 50.082 | 1.00 | 33.41 |
| ATOM | 1357 | CD | GLU | 181 | 2.419 | 30.986 | 49.064 | 1.00 | 35.66 |
| ATOM | 1358 | OE1 | GLU | 181 | 2.297 | 29.747 | 48.986 | 1.00 | 35.41 |
| ATOM | 1359 | OE2 | GLU | 181 | 3.213 | 31.632 | 48.349 | 1.00 | 38.36 |
| ATOM | 1360 | C | GLU | 181 | −0.821 | 33.477 | 50.709 | 1.00 | 30.13 |
| ATOM | 1361 | O | GLU | 181 | −1.465 | 33.809 | 49.714 | 1.00 | 31.04 |
| ATOM | 1362 | N | CYS | 182 | −0.154 | 34.337 | 51.474 | 1.00 | 30.42 |
| ATOM | 1363 | CA | CYS | 182 | −0.097 | 35.764 | 51.195 | 1.00 | 31.63 |
| ATOM | 1364 | CB | CYS | 182 | 0.946 | 36.026 | 50.111 | 1.00 | 31.93 |
| ATOM | 1365 | SG | CYS | 182 | 2.594 | 35.443 | 50.588 | 1.00 | 36.42 |
| ATOM | 1366 | C | CYS | 182 | −1.430 | 36.382 | 50.803 | 1.00 | 31.83 |
| ATOM | 1367 | O | CYS | 182 | −1.683 | 36.677 | 49.632 | 1.00 | 30.94 |
| ATOM | 1368 | N | VAL | 183 | −2.273 | 36.580 | 51.807 | 1.00 | 31.93 |
| ATOM | 1369 | CA | VAL | 183 | −3.587 | 37.165 | 51.614 | 1.00 | 34.24 |
| ATOM | 1370 | CB | VAL | 183 | −4.674 | 36.054 | 51.575 | 1.00 | 33.56 |
| ATOM | 1371 | CG1 | VAL | 183 | −4.945 | 35.517 | 52.974 | 1.00 | 34.62 |
| ATOM | 1372 | CG2 | VAL | 183 | −5.936 | 36.580 | 50.944 | 1.00 | 35.73 |
| ATOM | 1373 | C | VAL | 183 | −3.846 | 38.118 | 52.786 | 1.00 | 35.32 |
| ATOM | 1374 | O | VAL | 183 | −3.400 | 37.870 | 53.909 | 1.00 | 36.95 |
| ATOM | 1375 | N | PRO | 184 | −4.556 | 39.232 | 52.538 | 1.00 | 35.89 |
| ATOM | 1376 | CD | PRO | 184 | −5.239 | 39.654 | 51.302 | 1.00 | 35.39 |
| ATOM | 1377 | CA | PRO | 184 | −4.827 | 40.170 | 53.631 | 1.00 | 34.72 |
| ATOM | 1378 | CB | PRO | 184 | −5.751 | 41.203 | 52.980 | 1.00 | 35.50 |
| ATOM | 1379 | CG | PRO | 184 | −6.394 | 40.449 | 51.849 | 1.00 | 36.10 |
| ATOM | 1380 | C | PRO | 184 | −5.458 | 39.476 | 54.834 | 1.00 | 33.29 |
| ATOM | 1381 | O | PRO | 184 | −6.384 | 38.679 | 54.688 | 1.00 | 33.08 |
| ATOM | 1382 | N | VAL | 185 | −4.945 | 39.784 | 56.022 | 1.00 | 33.66 |
| ATOM | 1383 | CA | VAL | 185 | −5.440 | 39.184 | 57.261 | 1.00 | 33.09 |
| ATOM | 1384 | CB | VAL | 185 | −4.917 | 39.936 | 58.504 | 1.00 | 33.17 |
| ATOM | 1385 | CG1 | VAL | 185 | −5.309 | 39.182 | 59.763 | 1.00 | 33.65 |
| ATOM | 1386 | CG2 | VAL | 185 | −3.415 | 40.099 | 58.430 | 1.00 | 31.75 |
| ATOM | 1387 | C | VAL | 185 | −6.960 | 39.165 | 57.338 | 1.00 | 33.78 |
| ATOM | 1388 | O | VAL | 185 | −7.559 | 38.166 | 57.739 | 1.00 | 33.51 |
| ATOM | 1389 | N | GLU | 186 | −7.577 | 40.282 | 56.968 | 1.00 | 35.07 |
| ATOM | 1390 | CA | GLU | 186 | −9.030 | 40.407 | 56.991 | 1.00 | 36.33 |
| ATOM | 1391 | CB | GLU | 186 | −9.445 | 41.766 | 56.417 | 1.00 | 37.40 |
| ATOM | 1392 | CG | GLU | 186 | −8.658 | 42.181 | 55.181 | 1.00 | 42.15 |
| ATOM | 1393 | CD | GLU | 186 | −7.468 | 43.080 | 55.508 | 1.00 | 44.18 |
| ATOM | 1394 | OE1 | GLU | 186 | −6.790 | 42.840 | 56.533 | 1.00 | 43.77 |
| ATOM | 1395 | OE2 | GLU | 186 | −7.205 | 44.022 | 54.726 | 1.00 | 45.83 |
| ATOM | 1396 | C | GLU | 186 | −9.712 | 39.289 | 56.208 | 1.00 | 35.05 |
| ATOM | 1397 | O | GLU | 186 | −10.704 | 38.714 | 56.659 | 1.00 | 35.07 |
| ATOM | 1398 | N | LEU | 187 | −9.178 | 38.986 | 55.031 | 1.00 | 34.38 |
| ATOM | 1399 | CA | LEU | 187 | −9.743 | 37.941 | 54.188 | 1.00 | 33.21 |
| ATOM | 1400 | CB | LEU | 187 | −9.079 | 37.971 | 52.809 | 1.00 | 35.04 |
| ATOM | 1401 | CG | LEU | 187 | −9.927 | 37.542 | 51.607 | 1.00 | 36.74 |
| ATOM | 1402 | CD1 | LEU | 187 | −9.075 | 37.607 | 50.351 | 1.00 | 37.59 |
| ATOM | 1403 | CD2 | LEU | 187 | −10.475 | 36.146 | 51.807 | 1.00 | 36.68 |
| ATOM | 1404 | C | LEU | 187 | −9.533 | 36.577 | 54.836 | 1.00 | 32.03 |
| ATOM | 1405 | O | LEU | 187 | −10.431 | 35.735 | 54.839 | 1.00 | 30.51 |
| ATOM | 1406 | N | ALA | 188 | −8.340 | 36.364 | 55.386 | 1.00 | 31.07 |
| ATOM | 1407 | CA | ALA | 188 | −8.012 | 35.102 | 56.039 | 1.00 | 31.01 |
| ATOM | 1408 | CB | ALA | 188 | −6.576 | 35.138 | 56.554 | 1.00 | 30.06 |
| ATOM | 1409 | C | ALA | 188 | −8.979 | 34.822 | 57.182 | 1.00 | 30.77 |
| ATOM | 1410 | O | ALA | 188 | −9.345 | 33.673 | 57.424 | 1.00 | 30.09 |
| ATOM | 1411 | N | LYS | 189 | −9.396 | 35.880 | 57.876 | 1.00 | 32.19 |
| ATOM | 1412 | CA | LYS | 189 | −10.332 | 35.755 | 58.992 | 1.00 | 33.17 |
| ATOM | 1413 | CB | LYS | 189 | −10.573 | 37.119 | 59.653 | 1.00 | 35.50 |
| ATOM | 1414 | CG | LYS | 189 | −9.321 | 37.829 | 60.131 | 1.00 | 41.20 |
| ATOM | 1415 | CD | LYS | 189 | −9.644 | 39.151 | 60.826 | 1.00 | 44.52 |
| ATOM | 1416 | CE | LYS | 189 | −8.375 | 39.807 | 61.361 | 1.00 | 46.20 |
| ATOM | 1417 | NZ | LYS | 189 | −8.623 | 41.120 | 62.025 | 1.00 | 49.12 |
| ATOM | 1418 | C | LYS | 189 | −11.672 | 35.201 | 58.520 | 1.00 | 32.13 |
| ATOM | 1419 | O | LYS | 189 | −12.226 | 34.284 | 59.130 | 1.00 | 32.62 |
| ATOM | 1420 | N | ARG | 190 | −12.193 | 35.772 | 57.437 | 1.00 | 32.40 |
| ATOM | 1421 | CA | ARG | 190 | −13.478 | 35.343 | 56.887 | 1.00 | 33.08 |
| ATOM | 1422 | CB | ARG | 190 | −13.839 | 36.172 | 55.650 | 1.00 | 34.06 |
| ATOM | 1423 | CG | ARG | 190 | −14.021 | 37.652 | 55.906 | 1.00 | 37.30 |
| ATOM | 1424 | CD | ARG | 190 | −14.906 | 38.274 | 54.834 | 1.00 | 38.35 |
| ATOM | 1425 | NE | ARG | 190 | −14.261 | 38.346 | 53.528 | 1.00 | 38.64 |
| ATOM | 1426 | CZ | ARG | 190 | −14.920 | 38.343 | 52.373 | 1.00 | 38.37 |
| ATOM | 1427 | NH1 | ARG | 190 | −16.242 | 38.262 | 52.364 | 1.00 | 37.74 |
| ATOM | 1428 | NH2 | ARG | 190 | −14.265 | 38.435 | 51.223 | 1.00 | 38.25 |
| ATOM | 1429 | C | ARG | 190 | −13.480 | 33.869 | 56.508 | 1.00 | 31.06 |
| ATOM | 1430 | O | ARG | 190 | −14.363 | 33.115 | 56.913 | 1.00 | 31.92 |
| ATOM | 1431 | N | ILE | 191 | −12.488 | 33.466 | 55.723 | 1.00 | 31.60 |
| ATOM | 1432 | CA | ILE | 191 | −12.391 | 32.081 | 55.283 | 1.00 | 30.50 |
| ATOM | 1433 | CB | ILE | 191 | −11.197 | 31.887 | 54.322 | 1.00 | 32.14 |
| ATOM | 1434 | CG2 | ILE | 191 | −11.045 | 30.412 | 53.965 | 1.00 | 32.40 |
| ATOM | 1435 | CG1 | ILE | 191 | −11.415 | 32.724 | 53.057 | 1.00 | 32.62 |
| ATOM | 1436 | CD1 | ILE | 191 | −10.227 | 32.763 | 52.116 | 1.00 | 33.46 |
| ATOM | 1437 | C | ILE | 191 | −12.245 | 31.133 | 56.466 | 1.00 | 29.60 |
| ATOM | 1438 | O | ILE | 191 | −12.885 | 30.084 | 56.509 | 1.00 | 29.78 |
| ATOM | 1439 | N | THR | 192 | −11.410 | 31.509 | 57.428 | 1.00 | 28.46 |
| ATOM | 1440 | CA | THR | 192 | −11.190 | 30.673 | 58.597 | 1.00 | 28.93 |
| ATOM | 1441 | CB | THR | 192 | −10.100 | 31.269 | 59.514 | 1.00 | 28.24 |
| ATOM | 1442 | OG1 | THR | 192 | −8.870 | 31.379 | 58.788 | 1.00 | 28.74 |
| ATOM | 1443 | CG2 | THR | 192 | −9.878 | 30.380 | 60.723 | 1.00 | 27.20 |
| ATOM | 1444 | C | THR | 192 | −12.472 | 30.478 | 59.402 | 1.00 | 29.99 |
| ATOM | 1445 | O | THR | 192 | −12.747 | 29.378 | 59.885 | 1.00 | 28.00 |
| ATOM | 1446 | N | GLU | 193 | −13.257 | 31.542 | 59.548 | 1.00 | 31.93 |
| ATOM | 1447 | CA | GLU | 193 | −14.507 | 31.451 | 60.295 | 1.00 | 33.82 |
| ATOM | 1448 | CB | GLU | 193 | −15.021 | 32.845 | 60.666 | 1.00 | 36.65 |
| ATOM | 1449 | CG | GLU | 193 | −14.225 | 33.543 | 61.751 | 1.00 | 41.54 |
| ATOM | 1450 | CD | GLU | 193 | −14.789 | 34.912 | 62.097 | 1.00 | 44.86 |
| ATOM | 1451 | OE1 | GLU | 193 | −14.740 | 35.816 | 61.233 | 1.00 | 45.51 |
| ATOM | 1452 | OE2 | GLU | 193 | −15.284 | 35.083 | 63.236 | 1.00 | 47.21 |
| ATOM | 1453 | C | GLU | 193 | −15.567 | 30.725 | 59.480 | 1.00 | 32.10 |
| ATOM | 1454 | O | GLU | 193 | −16.372 | 29.974 | 60.023 | 1.00 | 33.17 |
| ATOM | 1455 | N | ALA | 194 | −15.554 | 30.947 | 58.172 | 1.00 | 31.90 |
| ATOM | 1456 | CA | ALA | 194 | −16.523 | 30.327 | 57.278 | 1.00 | 30.74 |
| ATOM | 1457 | CB | ALA | 194 | −16.446 | 30.980 | 55.910 | 1.00 | 30.76 |
| ATOM | 1458 | C | ALA | 194 | −16.352 | 28.818 | 57.140 | 1.00 | 30.69 |
| ATOM | 1459 | O | ALA | 194 | −17.338 | 28.083 | 57.051 | 1.00 | 30.09 |
| ATOM | 1460 | N | LEU | 195 | −15.106 | 28.353 | 57.123 | 1.00 | 29.16 |
| ATOM | 1461 | CA | LEU | 195 | −14.848 | 26.928 | 56.967 | 1.00 | 27.62 |
| ATOM | 1462 | CB | LEU | 195 | −13.555 | 26.704 | 56.173 | 1.00 | 28.66 |
| ATOM | 1463 | CG | LEU | 195 | −13.422 | 27.386 | 54.805 | 1.00 | 29.78 |
| ATOM | 1464 | CD1 | LEU | 195 | −12.296 | 26.713 | 54.020 | 1.00 | 28.71 |
| ATOM | 1465 | CD2 | LEU | 195 | −14.723 | 27.288 | 54.034 | 1.00 | 30.26 |
| ATOM | 1466 | C | LEU | 195 | −14.774 | 26.156 | 58.279 | 1.00 | 26.86 |
| ATOM | 1467 | O | LEU | 195 | −14.280 | 26.658 | 59.290 | 1.00 | 26.51 |
| ATOM | 1468 | N | ALA | 196 | −15.289 | 24.931 | 58.247 | 1.00 | 24.22 |
| ATOM | 1469 | CA | ALA | 196 | −15.282 | 24.046 | 59.403 | 1.00 | 24.93 |
| ATOM | 1470 | CB | ALA | 196 | −16.392 | 23.011 | 59.275 | 1.00 | 25.42 |
| ATOM | 1471 | C | ALA | 196 | −13.928 | 23.351 | 59.464 | 1.00 | 25.63 |
| ATOM | 1472 | O | ALA | 196 | −13.461 | 22.973 | 60.537 | 1.00 | 24.87 |
| ATOM | 1473 | N | ILE | 197 | −13.307 | 23.174 | 58.299 | 1.00 | 25.00 |
| ATOM | 1474 | CA | ILE | 197 | −12.003 | 22.527 | 58.225 | 1.00 | 23.95 |
| ATOM | 1475 | CB | ILE | 197 | −11.698 | 22.011 | 56.800 | 1.00 | 22.76 |
| ATOM | 1476 | CG2 | ILE | 197 | −12.670 | 20.901 | 56.426 | 1.00 | 21.58 |
| ATOM | 1477 | CG1 | ILE | 197 | −11.782 | 23.156 | 55.796 | 1.00 | 22.43 |
| ATOM | 1478 | CD1 | ILE | 197 | −11.166 | 22.818 | 54.447 | 1.00 | 24.79 |
| ATOM | 1479 | C | ILE | 197 | −10.910 | 23.505 | 58.628 | 1.00 | 24.38 |
| ATOM | 1480 | O | ILE | 197 | −11.011 | 24.709 | 58.381 | 1.00 | 24.17 |
| ATOM | 1481 | N | PRO | 198 | −9.845 | 23.001 | 59.260 | 1.00 | 24.33 |
| ATOM | 1482 | CD | PRO | 198 | −9.578 | 21.616 | 59.681 | 1.00 | 23.43 |
| ATOM | 1483 | CA | PRO | 198 | −8.768 | 23.903 | 59.668 | 1.00 | 24.80 |
| ATOM | 1484 | CB | PRO | 198 | −7.840 | 22.994 | 60.482 | 1.00 | 24.52 |
| ATOM | 1485 | CG | PRO | 198 | −8.104 | 21.638 | 59.926 | 1.00 | 26.07 |
| ATOM | 1486 | C | PRO | 198 | −8.070 | 24.579 | 58.489 | 1.00 | 23.71 |
| ATOM | 1487 | O | PRO | 198 | −7.840 | 23.965 | 57.445 | 1.00 | 24.75 |
| ATOM | 1488 | N | VAL | 199 | −7.765 | 25.859 | 58.659 | 1.00 | 22.66 |
| ATOM | 1489 | CA | VAL | 199 | −7.090 | 26.628 | 57.633 | 1.00 | 21.26 |
| ATOM | 1490 | CB | VAL | 199 | −7.840 | 27.952 | 57.343 | 1.00 | 21.86 |
| ATOM | 1491 | CG1 | VAL | 199 | −7.104 | 28.753 | 56.286 | 1.00 | 21.68 |
| ATOM | 1492 | CG2 | VAL | 199 | −9.261 | 27.651 | 56.881 | 1.00 | 23.56 |
| ATOM | 1493 | C | VAL | 199 | −5.677 | 26.948 | 58.113 | 1.00 | 22.90 |
| ATOM | 1494 | O | VAL | 199 | −5.489 | 27.514 | 59.191 | 1.00 | 21.18 |
| ATOM | 1495 | N | ILE | 200 | −4.689 | 26.568 | 57.311 | 1.00 | 21.02 |
| ATOM | 1496 | CA | ILE | 200 | −3.286 | 26.799 | 57.633 | 1.00 | 20.82 |
| ATOM | 1497 | CB | ILE | 200 | −2.425 | 25.579 | 57.265 | 1.00 | 22.17 |
| ATOM | 1498 | CG2 | ILE | 200 | −0.956 | 25.857 | 57.584 | 1.00 | 21.54 |
| ATOM | 1499 | CG1 | ILE | 200 | −2.925 | 24.346 | 58.015 | 1.00 | 21.31 |
| ATOM | 1500 | CD1 | ILE | 200 | −2.202 | 23.066 | 57.627 | 1.00 | 26.13 |
| ATOM | 1501 | C | ILE | 200 | −2.800 | 27.984 | 56.821 | 1.00 | 21.27 |
| ATOM | 1502 | O | ILE | 200 | −2.820 | 27.952 | 55.590 | 1.00 | 19.70 |
| ATOM | 1503 | N | GLY | 201 | −2.344 | 29.025 | 57.505 | 1.00 | 21.83 |
| ATOM | 1504 | CA | GLY | 201 | −1.883 | 30.199 | 56.791 | 1.00 | 21.11 |
| ATOM | 1505 | C | GLY | 201 | −0.382 | 30.376 | 56.722 | 1.00 | 21.20 |
| ATOM | 1506 | O | GLY | 201 | 0.360 | 29.915 | 57.586 | 1.00 | 20.91 |
| ATOM | 1507 | N | ILE | 202 | 0.053 | 31.035 | 55.656 | 1.00 | 20.88 |
| ATOM | 1508 | CA | ILE | 202 | 1.449 | 31.357 | 55.434 | 1.00 | 23.63 |
| ATOM | 1509 | CB | ILE | 202 | 2.135 | 30.374 | 54.442 | 1.00 | 25.53 |
| ATOM | 1510 | CG2 | ILE | 202 | 1.199 | 30.026 | 53.295 | 1.00 | 25.11 |
| ATOM | 1511 | CG1 | ILE | 202 | 3.431 | 30.991 | 53.922 | 1.00 | 26.37 |
| ATOM | 1512 | CD1 | ILE | 202 | 4.537 | 31.025 | 54.938 | 1.00 | 25.90 |
| ATOM | 1513 | C | ILE | 202 | 1.401 | 32.754 | 54.827 | 1.00 | 23.67 |
| ATOM | 1514 | O | ILE | 202 | 1.098 | 32.921 | 53.647 | 1.00 | 24.24 |
| ATOM | 1515 | N | GLY | 203 | 1.672 | 33.760 | 55.649 | 1.00 | 24.85 |
| ATOM | 1516 | CA | GLY | 203 | 1.615 | 35.130 | 55.177 | 1.00 | 24.91 |
| ATOM | 1517 | C | GLY | 203 | 0.167 | 35.580 | 55.162 | 1.00 | 24.72 |
| ATOM | 1518 | O | GLY | 203 | −0.222 | 36.447 | 54.378 | 1.00 | 26.70 |
| ATOM | 1519 | N | ALA | 204 | −0.635 | 34.981 | 56.037 | 1.00 | 24.39 |
| ATOM | 1520 | CA | ALA | 204 | −2.054 | 35.308 | 56.132 | 1.00 | 25.25 |
| ATOM | 1521 | CB | ALA | 204 | −2.889 | 34.113 | 55.704 | 1.00 | 25.27 |
| ATOM | 1522 | C | ALA | 204 | −2.467 | 35.745 | 57.538 | 1.00 | 25.30 |
| ATOM | 1523 | O | ALA | 204 | −3.648 | 35.701 | 57.885 | 1.00 | 26.25 |
| ATOM | 1524 | N | GLY | 205 | −1.495 | 36.155 | 58.346 | 1.00 | 26.07 |
| ATOM | 1525 | CA | GLY | 205 | −1.798 | 36.597 | 59.699 | 1.00 | 25.17 |
| ATOM | 1526 | C | GLY | 205 | −1.952 | 35.459 | 60.688 | 1.00 | 25.65 |
| ATOM | 1527 | O | GLY | 205 | −1.853 | 34.294 | 60.316 | 1.00 | 24.90 |
| ATOM | 1528 | N | ASN | 206 | −2.195 | 35.791 | 61.952 | 1.00 | 24.53 |
| ATOM | 1529 | CA | ASN | 206 | −2.350 | 34.772 | 62.983 | 1.00 | 24.06 |
| ATOM | 1530 | CB | ASN | 206 | −1.739 | 35.250 | 64.298 | 1.00 | 25.67 |
| ATOM | 1531 | CG | ASN | 206 | −2.512 | 36.405 | 64.915 | 1.00 | 27.21 |
| ATOM | 1532 | OD1 | ASN | 206 | −2.316 | 36.744 | 66.081 | 1.00 | 27.76 |
| ATOM | 1533 | ND2 | ASN | 206 | −3.393 | 37.016 | 64.131 | 1.00 | 24.74 |
| ATOM | 1534 | C | ASN | 206 | −3.805 | 34.407 | 63.230 | 1.00 | 22.53 |
| ATOM | 1535 | O | ASN | 206 | −4.127 | 33.788 | 64.246 | 1.00 | 21.69 |
| ATOM | 1536 | N | VAL | 207 | −4.680 | 34.786 | 62.305 | 1.00 | 21.94 |
| ATOM | 1537 | CA | VAL | 207 | −6.098 | 34.496 | 62.446 | 1.00 | 23.84 |
| ATOM | 1538 | CB | VAL | 207 | −6.953 | 35.513 | 61.668 | 1.00 | 26.20 |
| ATOM | 1539 | CG1 | VAL | 207 | −8.423 | 35.336 | 62.037 | 1.00 | 29.08 |
| ATOM | 1540 | CG2 | VAL | 207 | −6.488 | 36.925 | 61.981 | 1.00 | 27.68 |
| ATOM | 1541 | C | VAL | 207 | −6.453 | 33.091 | 61.965 | 1.00 | 24.32 |
| ATOM | 1542 | O | VAL | 207 | −7.563 | 32.600 | 62.203 | 1.00 | 23.30 |
| ATOM | 1543 | N | THR | 208 | −5.513 | 32.431 | 61.297 | 1.00 | 23.32 |
| ATOM | 1544 | CA | THR | 208 | −5.779 | 31.086 | 60.811 | 1.00 | 23.51 |
| ATOM | 1545 | CB | THR | 208 | −4.840 | 30.708 | 59.643 | 1.00 | 23.12 |
| ATOM | 1546 | OG1 | THR | 208 | −3.481 | 30.977 | 60.008 | 1.00 | 21.10 |
| ATOM | 1547 | CG2 | THR | 208 | −5.198 | 31.502 | 58.402 | 1.00 | 23.13 |
| ATOM | 1548 | C | THR | 208 | −5.663 | 30.053 | 61.919 | 1.00 | 24.29 |
| ATOM | 1549 | O | THR | 208 | −5.129 | 30.331 | 62.995 | 1.00 | 24.25 |
| ATOM | 1550 | N | ASP | 209 | −6.185 | 28.861 | 61.657 | 1.00 | 22.58 |
| ATOM | 1551 | CA | ASP | 209 | −6.164 | 27.788 | 62.642 | 1.00 | 22.69 |
| ATOM | 1552 | CB | ASP | 209 | −7.091 | 26.665 | 62.194 | 1.00 | 22.67 |
| ATOM | 1553 | CG | ASP | 209 | −8.501 | 27.146 | 61.961 | 1.00 | 25.87 |
| ATOM | 1554 | OD1 | ASP | 209 | −9.130 | 27.621 | 62.931 | 1.00 | 23.71 |
| ATOM | 1555 | OD2 | ASP | 209 | −8.980 | 27.056 | 60.808 | 1.00 | 24.21 |
| ATOM | 1556 | C | ASP | 209 | −4.764 | 27.244 | 62.830 | 1.00 | 21.49 |
| ATOM | 1557 | O | ASP | 209 | −4.451 | 26.657 | 63.863 | 1.00 | 21.81 |
| ATOM | 1558 | N | GLY | 210 | −3.928 | 27.444 | 61.819 | 1.00 | 21.84 |
| ATOM | 1559 | CA | GLY | 210 | −2.569 | 26.956 | 61.891 | 1.00 | 20.56 |
| ATOM | 1560 | C | GLY | 210 | −1.623 | 27.775 | 61.043 | 1.00 | 20.58 |
| ATOM | 1561 | O | GLY | 210 | −2.049 | 28.651 | 60.287 | 1.00 | 21.05 |
| ATOM | 1562 | N | GLN | 211 | −0.332 | 27.473 | 61.156 | 1.00 | 20.83 |
| ATOM | 1563 | CA | GLN | 211 | 0.699 | 28.192 | 60.424 | 1.00 | 21.52 |
| ATOM | 1564 | CB | GLN | 211 | 1.459 | 29.119 | 61.379 | 1.00 | 22.07 |
| ATOM | 1565 | CG | GLN | 211 | 0.626 | 30.239 | 61.993 | 1.00 | 19.78 |
| ATOM | 1566 | CD | GLN | 211 | 0.135 | 31.229 | 60.960 | 1.00 | 21.63 |
| ATOM | 1567 | OE1 | GLN | 211 | 0.895 | 31.663 | 60.093 | 1.00 | 23.63 |
| ATOM | 1568 | NE2 | GLN | 211 | −1.139 | 31.608 | 61.055 | 1.00 | 21.71 |
| ATOM | 1569 | C | GLN | 211 | 1.701 | 27.250 | 59.761 | 1.00 | 21.50 |
| ATOM | 1570 | O | GLN | 211 | 1.886 | 26.117 | 60.199 | 1.00 | 20.81 |
| ATOM | 1571 | N | ILE | 212 | 2.344 | 27.724 | 58.700 | 1.00 | 22.48 |
| ATOM | 1572 | CA | ILE | 212 | 3.351 | 26.921 | 58.023 | 1.00 | 26.35 |
| ATOM | 1573 | CB | ILE | 212 | 2.755 | 26.171 | 56.799 | 1.00 | 28.17 |
| ATOM | 1574 | CG2 | ILE | 212 | 2.484 | 27.139 | 55.660 | 1.00 | 29.37 |
| ATOM | 1575 | CG1 | ILE | 212 | 3.725 | 25.075 | 56.344 | 1.00 | 29.11 |
| ATOM | 1576 | CD1 | ILE | 212 | 3.081 | 24.015 | 55.457 | 1.00 | 31.12 |
| ATOM | 1577 | C | ILE | 212 | 4.494 | 27.829 | 57.588 | 1.00 | 28.11 |
| ATOM | 1578 | O | ILE | 212 | 4.287 | 29.011 | 57.319 | 1.00 | 29.09 |
| ATOM | 1579 | N | LEU | 213 | 5.706 | 27.287 | 57.556 | 1.00 | 29.45 |
| ATOM | 1580 | CA | LEU | 213 | 6.870 | 28.062 | 57.135 | 1.00 | 31.99 |
| ATOM | 1581 | CB | LEU | 213 | 7.432 | 28.880 | 58.300 | 1.00 | 33.40 |
| ATOM | 1582 | CG | LEU | 213 | 7.351 | 30.415 | 58.241 | 1.00 | 33.83 |
| ATOM | 1583 | CD1 | LEU | 213 | 8.119 | 30.972 | 59.435 | 1.00 | 32.93 |
| ATOM | 1584 | CD2 | LEU | 213 | 7.941 | 30.952 | 56.936 | 1.00 | 31.79 |
| ATOM | 1585 | C | LEU | 213 | 7.970 | 27.159 | 56.602 | 1.00 | 32.15 |
| ATOM | 1586 | O | LEU | 213 | 8.143 | 26.033 | 57.076 | 1.00 | 31.38 |
| ATOM | 1587 | N | VAL | 214 | 8.699 | 27.656 | 55.607 | 1.00 | 31.01 |
| ATOM | 1588 | CA | VAL | 214 | 9.808 | 26.913 | 55.025 | 1.00 | 30.31 |
| ATOM | 1589 | CB | VAL | 214 | 10.300 | 27.550 | 53.695 | 1.00 | 31.87 |
| ATOM | 1590 | CG1 | VAL | 214 | 11.516 | 26.796 | 53.174 | 1.00 | 32.34 |
| ATOM | 1591 | CG2 | VAL | 214 | 9.188 | 27.505 | 52.659 | 1.00 | 33.39 |
| ATOM | 1592 | C | VAL | 214 | 10.923 | 26.976 | 56.060 | 1.00 | 28.15 |
| ATOM | 1593 | O | VAL | 214 | 11.440 | 28.050 | 56.362 | 1.00 | 28.96 |
| ATOM | 1594 | N | MET | 215 | 11.268 | 25.820 | 56.614 | 1.00 | 25.80 |
| ATOM | 1595 | CA | MET | 215 | 12.299 | 25.727 | 57.632 | 1.00 | 24.04 |
| ATOM | 1596 | CB | MET | 215 | 12.617 | 24.254 | 57.927 | 1.00 | 21.67 |
| ATOM | 1597 | CG | MET | 215 | 13.046 | 23.449 | 56.708 | 1.00 | 21.44 |
| ATOM | 1598 | SD | MET | 215 | 14.311 | 22.218 | 57.084 | 1.00 | 19.28 |
| ATOM | 1599 | CE | MET | 215 | 15.756 | 23.248 | 56.942 | 1.00 | 20.16 |
| ATOM | 1600 | C | MET | 215 | 13.575 | 26.465 | 57.253 | 1.00 | 23.77 |
| ATOM | 1601 | O | MET | 215 | 14.253 | 27.025 | 58.114 | 1.00 | 24.19 |
| ATOM | 1602 | N | HIS | 216 | 13.901 | 26.489 | 55.966 | 1.00 | 23.61 |
| ATOM | 1603 | CA | HIS | 216 | 15.122 | 27.163 | 55.520 | 1.00 | 25.02 |
| ATOM | 1604 | CB | HIS | 216 | 15.342 | 26.890 | 54.035 | 1.00 | 25.17 |
| ATOM | 1605 | CG | HIS | 216 | 15.707 | 25.468 | 53.751 | 1.00 | 25.07 |
| ATOM | 1606 | CD2 | HIS | 216 | 14.939 | 24.360 | 53.621 | 1.00 | 22.61 |
| ATOM | 1607 | ND1 | HIS | 216 | 17.014 | 25.039 | 53.656 | 1.00 | 24.95 |
| ATOM | 1608 | CE1 | HIS | 216 | 17.035 | 23.730 | 53.479 | 1.00 | 23.43 |
| ATOM | 1609 | NE2 | HIS | 216 | 15.788 | 23.294 | 53.455 | 1.00 | 26.42 |
| ATOM | 1610 | C | HIS | 216 | 15.132 | 28.659 | 55.812 | 1.00 | 26.05 |
| ATOM | 1611 | O | HIS | 216 | 16.195 | 29.277 | 55.872 | 1.00 | 25.85 |
| ATOM | 1612 | N | ASP | 217 | 13.949 | 29.238 | 56.000 | 1.00 | 27.68 |
| ATOM | 1613 | CA | ASP | 217 | 13.848 | 30.661 | 56.321 | 1.00 | 31.25 |
| ATOM | 1614 | CB | ASP | 217 | 12.576 | 31.271 | 55.713 | 1.00 | 33.06 |
| ATOM | 1615 | CG | ASP | 217 | 12.653 | 31.406 | 54.200 | 1.00 | 35.27 |
| ATOM | 1616 | OD1 | ASP | 217 | 13.623 | 32.016 | 53.700 | 1.00 | 36.27 |
| ATOM | 1617 | OD2 | ASP | 217 | 11.735 | 30.916 | 53.507 | 1.00 | 37.69 |
| ATOM | 1618 | C | ASP | 217 | 13.827 | 30.825 | 57.845 | 1.00 | 31.02 |
| ATOM | 1619 | O | ASP | 217 | 14.353 | 31.803 | 58.394 | 1.00 | 32.35 |
| ATOM | 1620 | N | ALA | 218 | 13.225 | 29.853 | 58.524 | 1.00 | 30.86 |
| ATOM | 1621 | CA | ALA | 218 | 13.121 | 29.882 | 59.981 | 1.00 | 30.59 |
| ATOM | 1622 | CB | ALA | 218 | 12.208 | 28.761 | 60.451 | 1.00 | 30.22 |
| ATOM | 1623 | C | ALA | 218 | 14.471 | 29.783 | 60.689 | 1.00 | 31.33 |
| ATOM | 1624 | O | ALA | 218 | 14.593 | 30.180 | 61.851 | 1.00 | 31.23 |
| ATOM | 1625 | N | PHE | 219 | 15.482 | 29.267 | 59.992 | 1.00 | 30.28 |
| ATOM | 1626 | CA | PHE | 219 | 16.804 | 29.117 | 60.589 | 1.00 | 29.83 |
| ATOM | 1627 | CB | PHE | 219 | 17.202 | 27.640 | 60.598 | 1.00 | 29.92 |
| ATOM | 1628 | CG | PHE | 219 | 16.148 | 26.736 | 61.177 | 1.00 | 30.71 |
| ATOM | 1629 | CD1 | PHE | 219 | 15.529 | 27.052 | 62.385 | 1.00 | 31.14 |
| ATOM | 1630 | CD2 | PHE | 219 | 15.781 | 25.562 | 60.524 | 1.00 | 31.43 |
| ATOM | 1631 | CE1 | PHE | 219 | 14.562 | 26.213 | 62.935 | 1.00 | 31.73 |
| ATOM | 1632 | CE2 | PHE | 219 | 14.811 | 24.714 | 61.067 | 1.00 | 32.41 |
| ATOM | 1633 | CZ | PHE | 219 | 14.202 | 25.041 | 62.275 | 1.00 | 31.77 |
| ATOM | 1634 | C | PHE | 219 | 17.881 | 29.943 | 59.899 | 1.00 | 29.17 |
| ATOM | 1635 | O | PHE | 219 | 19.069 | 29.621 | 59.968 | 1.00 | 29.21 |
| ATOM | 1636 | N | GLY | 220 | 17.458 | 31.014 | 59.237 | 1.00 | 29.74 |
| ATOM | 1637 | CA | GLY | 220 | 18.390 | 31.889 | 58.550 | 1.00 | 29.75 |
| ATOM | 1638 | C | GLY | 220 | 19.357 | 31.185 | 57.621 | 1.00 | 30.84 |
| ATOM | 1639 | O | GLY | 220 | 20.507 | 31.601 | 57.485 | 1.00 | 30.29 |
| ATOM | 1640 | N | ILE | 221 | 18.900 | 30.120 | 56.973 | 1.00 | 30.94 |
| ATOM | 1641 | CA | ILE | 221 | 19.754 | 29.380 | 56.053 | 1.00 | 30.23 |
| ATOM | 1642 | CB | ILE | 221 | 19.270 | 27.930 | 55.896 | 1.00 | 28.54 |
| ATOM | 1643 | CG2 | ILE | 221 | 20.167 | 27.187 | 54.908 | 1.00 | 26.85 |
| ATOM | 1644 | CG1 | ILE | 221 | 19.288 | 27.239 | 57.263 | 1.00 | 27.41 |
| ATOM | 1645 | CD1 | ILE | 221 | 18.654 | 25.879 | 57.279 | 1.00 | 26.63 |
| ATOM | 1646 | C | ILE | 221 | 19.759 | 30.060 | 54.691 | 1.00 | 31.63 |
| ATOM | 1647 | O | ILE | 221 | 20.799 | 30.178 | 54.041 | 1.00 | 30.47 |
| ATOM | 1648 | N | THR | 222 | 18.590 | 30.528 | 54.275 | 1.00 | 33.36 |
| ATOM | 1649 | CA | THR | 222 | 18.453 | 31.193 | 52.989 | 1.00 | 37.25 |
| ATOM | 1650 | CB | THR | 222 | 16.981 | 31.241 | 52.555 | 1.00 | 36.65 |
| ATOM | 1651 | OG1 | THR | 222 | 16.249 | 32.088 | 53.448 | 1.00 | 39.18 |
| ATOM | 1652 | CG2 | THR | 222 | 16.375 | 29.851 | 52.594 | 1.00 | 35.68 |
| ATOM | 1653 | C | THR | 222 | 18.995 | 32.616 | 53.029 | 1.00 | 39.66 |
| ATOM | 1654 | O | THR | 222 | 18.770 | 33.345 | 53.993 | 1.00 | 39.16 |
| ATOM | 1655 | N | GLY | 223 | 19.713 | 32.990 | 51.970 | 1.00 | 43.16 |
| ATOM | 1656 | CA | GLY | 223 | 20.292 | 34.320 | 51.848 | 1.00 | 47.53 |
| ATOM | 1657 | C | GLY | 223 | 20.326 | 35.168 | 53.105 | 1.00 | 50.12 |
| ATOM | 1658 | O | GLY | 223 | 20.927 | 34.786 | 54.110 | 1.00 | 51.54 |
| ATOM | 1659 | N | GLY | 224 | 19.680 | 36.327 | 53.052 | 1.00 | 51.89 |
| ATOM | 1660 | CA | GLY | 224 | 19.663 | 37.202 | 54.207 | 1.00 | 54.05 |
| ATOM | 1661 | C | GLY | 224 | 18.324 | 37.871 | 54.422 | 1.00 | 56.18 |
| ATOM | 1662 | O | GLY | 224 | 17.767 | 37.824 | 55.520 | 1.00 | 56.72 |
| ATOM | 1663 | N | HIS | 225 | 17.802 | 38.495 | 53.371 | 1.00 | 57.60 |
| ATOM | 1664 | CA | HIS | 225 | 16.520 | 39.185 | 53.453 | 1.00 | 58.83 |
| ATOM | 1665 | CB | HIS | 225 | 16.487 | 40.359 | 52.470 | 1.00 | 61.50 |
| ATOM | 1666 | CG | HIS | 225 | 17.419 | 41.475 | 52.828 | 1.00 | 63.93 |
| ATOM | 1667 | CD2 | HIS | 225 | 18.446 | 42.036 | 52.146 | 1.00 | 65.25 |
| ATOM | 1668 | ND1 | HIS | 225 | 17.338 | 42.155 | 54.025 | 1.00 | 65.20 |
| ATOM | 1669 | CE1 | HIS | 225 | 18.275 | 43.087 | 54.063 | 1.00 | 65.92 |
| ATOM | 1670 | NE2 | HIS | 225 | 18.960 | 43.036 | 52.935 | 1.00 | 66.01 |
| ATOM | 1671 | C | HIS | 225 | 15.340 | 38.257 | 53.183 | 1.00 | 57.89 |
| ATOM | 1672 | O | HIS | 225 | 14.797 | 38.234 | 52.079 | 1.00 | 58.08 |
| ATOM | 1673 | N | ILE | 226 | 14.947 | 37.497 | 54.201 | 1.00 | 56.71 |
| ATOM | 1674 | CA | ILE | 226 | 13.825 | 36.573 | 54.080 | 1.00 | 55.07 |
| ATOM | 1675 | CB | ILE | 226 | 13.651 | 35.740 | 55.367 | 1.00 | 55.20 |
| ATOM | 1676 | CG2 | ILE | 226 | 14.888 | 34.885 | 55.601 | 1.00 | 54.98 |
| ATOM | 1677 | CG1 | ILE | 226 | 13.400 | 36.668 | 56.558 | 1.00 | 54.81 |
| ATOM | 1678 | CD1 | ILE | 226 | 13.109 | 35.942 | 57.852 | 1.00 | 54.53 |
| ATOM | 1679 | C | ILE | 226 | 12.541 | 37.359 | 53.820 | 1.00 | 53.36 |
| ATOM | 1680 | O | ILE | 226 | 12.499 | 38.576 | 54.020 | 1.00 | 54.02 |
| ATOM | 1681 | N | PRO | 227 | 11.473 | 36.674 | 53.377 | 1.00 | 50.99 |
| ATOM | 1682 | CD | PRO | 227 | 11.336 | 35.223 | 53.161 | 1.00 | 49.98 |
| ATOM | 1683 | CA | PRO | 227 | 10.206 | 37.361 | 53.104 | 1.00 | 48.87 |
| ATOM | 1684 | CB | PRO | 227 | 9.319 | 36.242 | 52.564 | 1.00 | 48.89 |
| ATOM | 1685 | CG | PRO | 227 | 9.847 | 35.031 | 53.259 | 1.00 | 49.85 |
| ATOM | 1686 | C | PRO | 227 | 9.615 | 38.045 | 54.336 | 1.00 | 46.39 |
| ATOM | 1687 | O | PRO | 227 | 9.860 | 37.626 | 55.465 | 1.00 | 46.32 |
| ATOM | 1688 | N | LYS | 228 | 8.839 | 39.101 | 54.108 | 1.00 | 44.27 |
| ATOM | 1689 | CA | LYS | 228 | 8.222 | 39.850 | 55.195 | 1.00 | 41.95 |
| ATOM | 1690 | CB | LYS | 228 | 7.360 | 40.992 | 54.648 | 1.00 | 43.42 |
| ATOM | 1691 | CG | LYS | 228 | 8.139 | 42.134 | 54.016 | 1.00 | 47.18 |
| ATOM | 1692 | CD | LYS | 228 | 8.750 | 41.736 | 52.678 | 1.00 | 49.90 |
| ATOM | 1693 | CE | LYS | 228 | 9.498 | 42.906 | 52.048 | 1.00 | 51.00 |
| ATOM | 1694 | NZ | LYS | 228 | 10.007 | 42.601 | 50.679 | 1.00 | 51.75 |
| ATOM | 1695 | C | LYS | 228 | 7.367 | 38.987 | 56.110 | 1.00 | 39.05 |
| ATOM | 1696 | O | LYS | 228 | 7.232 | 39.278 | 57.297 | 1.00 | 39.22 |
| ATOM | 1697 | N | PHE | 229 | 6.787 | 37.927 | 55.561 | 1.00 | 35.73 |
| ATOM | 1698 | CA | PHE | 229 | 5.935 | 37.052 | 56.352 | 1.00 | 31.21 |
| ATOM | 1699 | CB | PHE | 229 | 4.883 | 36.397 | 55.453 | 1.00 | 32.42 |
| ATOM | 1700 | CG | PHE | 229 | 5.460 | 35.543 | 54.360 | 1.00 | 32.78 |
| ATOM | 1701 | CD1 | PHE | 229 | 5.908 | 34.253 | 54.625 | 1.00 | 32.94 |
| ATOM | 1702 | CD2 | PHE | 229 | 5.554 | 36.030 | 53.060 | 1.00 | 34.15 |
| ATOM | 1703 | CE1 | PHE | 229 | 6.442 | 33.457 | 53.606 | 1.00 | 32.72 |
| ATOM | 1704 | CE2 | PHE | 229 | 6.086 | 35.246 | 52.034 | 1.00 | 33.39 |
| ATOM | 1705 | CZ | PHE | 229 | 6.529 | 33.958 | 52.309 | 1.00 | 33.05 |
| ATOM | 1706 | C | PHE | 229 | 6.707 | 35.985 | 57.116 | 1.00 | 28.23 |
| ATOM | 1707 | O | PHE | 229 | 6.126 | 35.262 | 57.921 | 1.00 | 28.17 |
| ATOM | 1708 | N | ALA | 230 | 8.013 | 35.900 | 56.876 | 1.00 | 26.34 |
| ATOM | 1709 | CA | ALA | 230 | 8.848 | 34.907 | 57.544 | 1.00 | 24.97 |
| ATOM | 1710 | CB | ALA | 230 | 9.833 | 34.304 | 56.552 | 1.00 | 26.85 |
| ATOM | 1711 | C | ALA | 230 | 9.605 | 35.484 | 58.734 | 1.00 | 26.01 |
| ATOM | 1712 | O | ALA | 230 | 9.743 | 36.700 | 58.865 | 1.00 | 25.21 |
| ATOM | 1713 | N | LYS | 231 | 10.090 | 34.596 | 59.600 | 1.00 | 24.27 |
| ATOM | 1714 | CA | LYS | 231 | 10.838 | 34.999 | 60.786 | 1.00 | 24.53 |
| ATOM | 1715 | CB | LYS | 231 | 9.898 | 35.149 | 61.989 | 1.00 | 25.49 |
| ATOM | 1716 | CG | LYS | 231 | 10.609 | 35.476 | 63.295 | 1.00 | 25.37 |
| ATOM | 1717 | CD | LYS | 231 | 9.634 | 35.596 | 64.455 | 1.00 | 28.18 |
| ATOM | 1718 | CE | LYS | 231 | 10.372 | 35.784 | 65.780 | 1.00 | 29.49 |
| ATOM | 1719 | NZ | LYS | 231 | 9.434 | 35.995 | 66.923 | 1.00 | 29.09 |
| ATOM | 1720 | C | LYS | 231 | 11.919 | 33.977 | 61.118 | 1.00 | 23.25 |
| ATOM | 1721 | O | LYS | 231 | 11.674 | 32.775 | 61.097 | 1.00 | 20.91 |
| ATOM | 1722 | N | ASN | 232 | 13.115 | 34.470 | 61.419 | 1.00 | 22.11 |
| ATOM | 1723 | CA | ASN | 232 | 14.238 | 33.617 | 61.775 | 1.00 | 22.68 |
| ATOM | 1724 | CB | ASN | 232 | 15.550 | 34.325 | 61.427 | 1.00 | 22.74 |
| ATOM | 1725 | CG | ASN | 232 | 16.770 | 33.476 | 61.711 | 1.00 | 22.28 |
| ATOM | 1726 | OD1 | ASN | 232 | 16.710 | 32.519 | 62.477 | 1.00 | 22.05 |
| ATOM | 1727 | ND2 | ASN | 232 | 17.897 | 33.839 | 61.103 | 1.00 | 21.01 |
| ATOM | 1728 | C | ASN | 232 | 14.157 | 33.363 | 63.283 | 1.00 | 22.41 |
| ATOM | 1729 | O | ASN | 232 | 14.442 | 34.256 | 64.083 | 1.00 | 22.40 |
| ATOM | 1730 | N | PHE | 233 | 13.754 | 32.158 | 63.670 | 1.00 | 20.06 |
| ATOM | 1731 | CA | PHE | 233 | 13.640 | 31.816 | 65.083 | 1.00 | 21.57 |
| ATOM | 1732 | CB | PHE | 233 | 12.623 | 30.692 | 65.284 | 1.00 | 20.88 |
| ATOM | 1733 | CG | PHE | 233 | 11.193 | 31.121 | 65.109 | 1.00 | 20.63 |
| ATOM | 1734 | CD1 | PHE | 233 | 10.614 | 31.161 | 63.849 | 1.00 | 21.32 |
| ATOM | 1735 | CD2 | PHE | 233 | 10.428 | 31.498 | 66.210 | 1.00 | 20.85 |
| ATOM | 1736 | CE1 | PHE | 233 | 9.289 | 31.566 | 63.678 | 1.00 | 21.06 |
| ATOM | 1737 | CE2 | PHE | 233 | 9.102 | 31.905 | 66.053 | 1.00 | 21.27 |
| ATOM | 1738 | CZ | PHE | 233 | 8.536 | 31.940 | 64.783 | 1.00 | 19.83 |
| ATOM | 1739 | C | PHE | 233 | 14.973 | 31.400 | 65.698 | 1.00 | 21.41 |
| ATOM | 1740 | O | PHE | 233 | 15.130 | 31.398 | 66.921 | 1.00 | 22.96 |
| ATOM | 1741 | N | LEU | 234 | 15.928 | 31.033 | 64.852 | 1.00 | 22.02 |
| ATOM | 1742 | CA | LEU | 234 | 17.242 | 30.622 | 65.328 | 1.00 | 24.52 |
| ATOM | 1743 | CB | LEU | 234 | 18.008 | 29.894 | 64.219 | 1.00 | 23.62 |
| ATOM | 1744 | CG | LEU | 234 | 19.465 | 29.538 | 64.524 | 1.00 | 22.78 |
| ATOM | 1745 | CD1 | LEU | 234 | 19.527 | 28.580 | 65.701 | 1.00 | 21.54 |
| ATOM | 1746 | CD2 | LEU | 234 | 20.112 | 28.923 | 63.292 | 1.00 | 25.26 |
| ATOM | 1747 | C | LEU | 234 | 18.045 | 31.838 | 65.773 | 1.00 | 26.95 |
| ATOM | 1748 | O | LEU | 234 | 18.727 | 31.801 | 66.796 | 1.00 | 25.49 |
| ATOM | 1749 | N | ALA | 235 | 17.958 | 32.911 | 64.996 | 1.00 | 30.52 |
| ATOM | 1750 | CA | ALA | 235 | 18.681 | 34.138 | 65.302 | 1.00 | 36.40 |
| ATOM | 1751 | CB | ALA | 235 | 18.274 | 35.239 | 64.331 | 1.00 | 37.16 |
| ATOM | 1752 | C | ALA | 235 | 18.417 | 34.578 | 66.736 | 1.00 | 40.32 |
| ATOM | 1753 | O | ALA | 235 | 19.337 | 34.973 | 67.454 | 1.00 | 40.96 |
| ATOM | 1754 | N | GLU | 236 | 17.154 | 34.501 | 67.145 | 1.00 | 43.98 |
| ATOM | 1755 | CA | GLU | 236 | 16.750 | 34.881 | 68.497 | 1.00 | 47.59 |
| ATOM | 1756 | CB | GLU | 236 | 15.280 | 34.514 | 68.730 | 1.00 | 48.96 |
| ATOM | 1757 | CG | GLU | 236 | 14.320 | 34.967 | 67.632 | 1.00 | 51.28 |
| ATOM | 1758 | CD | CLU | 236 | 14.177 | 36.477 | 67.544 | 1.00 | 52.90 |
| ATOM | 1759 | OE1 | GLU | 236 | 15.174 | 37.159 | 67.224 | 1.00 | 53.61 |
| ATOM | 1760 | OE2 | CLU | 236 | 13.063 | 36.984 | 67.798 | 1.00 | 53.70 |
| ATOM | 1761 | C | GLU | 236 | 17.625 | 34.138 | 69.502 | 1.00 | 48.66 |
| ATOM | 1762 | O | GLU | 236 | 18.234 | 34.748 | 70.380 | 1.00 | 49.42 |
| ATOM | 1763 | N | THR | 237 | 17.681 | 32.817 | 69.355 | 1.00 | 49.40 |
| ATOM | 1764 | CA | THR | 237 | 18.475 | 31.967 | 70.235 | 1.00 | 49.62 |
| ATOM | 1765 | CB | THR | 237 | 17.668 | 30.713 | 70.667 | 1.00 | 50.79 |
| ATOM | 1766 | OG1 | THR | 237 | 18.464 | 29.900 | 71.542 | 1.00 | 52.05 |
| ATOM | 1767 | CG2 | THR | 237 | 17.247 | 29.894 | 69.452 | 1.00 | 51.76 |
| ATOM | 1768 | C | THR | 237 | 19.772 | 31.529 | 69.548 | 1.00 | 48.44 |
| ATOM | 1769 | O | THR | 237 | 20.349 | 32.278 | 68.759 | 1.00 | 49.89 |
| ATOM | 1770 | N | GLY | 238 | 20.234 | 30.323 | 69.858 | 1.00 | 46.44 |
| ATOM | 1771 | CA | GLY | 238 | 21.451 | 29.819 | 69.248 | 1.00 | 42.67 |
| ATOM | 1772 | C | GLY | 238 | 21.379 | 28.318 | 69.059 | 1.00 | 41.05 |
| ATOM | 1773 | O | GLY | 238 | 22.385 | 27.663 | 68.790 | 1.00 | 41.30 |
| ATOM | 1774 | N | ASP | 239 | 20.174 | 27.776 | 69.194 | 1.00 | 37.96 |
| ATOM | 1775 | CA | ASP | 239 | 19.950 | 26.345 | 69.058 | 1.00 | 34.93 |
| ATOM | 1776 | CB | ASP | 239 | 19.748 | 25.735 | 70.449 | 1.00 | 38.61 |
| ATOM | 1777 | CG | ASP | 239 | 19.415 | 24.262 | 70.399 | 1.00 | 41.94 |
| ATOM | 1778 | OD1 | ASP | 239 | 18.237 | 23.928 | 70.170 | 1.00 | 42.16 |
| ATOM | 1779 | OD2 | ASP | 239 | 20.341 | 23.438 | 70.582 | 1.00 | 45.62 |
| ATOM | 1780 | C | ASP | 239 | 18.743 | 26.059 | 68.162 | 1.00 | 31.17 |
| ATOM | 1781 | O | ASP | 239 | 17.661 | 26.608 | 68.370 | 1.00 | 28.67 |
| ATOM | 1782 | N | ILE | 240 | 18.926 | 25.198 | 67.164 | 1.00 | 25.89 |
| ATOM | 1783 | CA | ILE | 240 | 17.834 | 24.882 | 66.252 | 1.00 | 22.33 |
| ATOM | 1784 | CB | ILE | 240 | 18.295 | 23.912 | 65.132 | 1.00 | 21.20 |
| ATOM | 1785 | CG2 | ILE | 240 | 17.099 | 23.518 | 64.256 | 1.00 | 17.39 |
| ATOM | 1786 | CG1 | ILE | 240 | 19.356 | 24.592 | 64.265 | 1.00 | 21.01 |
| ATOM | 1787 | CD1 | ILE | 240 | 20.042 | 23.665 | 63.267 | 1.00 | 21.65 |
| ATOM | 1788 | C | ILE | 240 | 16.609 | 24.312 | 66.970 | 1.00 | 19.27 |
| ATOM | 1789 | O | ILE | 240 | 15.495 | 24.756 | 66.719 | 1.00 | 21.10 |
| ATOM | 1790 | N | ARG | 241 | 16.808 | 23.352 | 67.870 | 1.00 | 19.73 |
| ATOM | 1791 | CA | ARG | 241 | 15.687 | 22.761 | 68.592 | 1.00 | 19.91 |
| ATOM | 1792 | CB | ARG | 241 | 16.167 | 21.604 | 69.472 | 1.00 | 18.67 |
| ATOM | 1793 | CG | ARG | 241 | 16.544 | 20.366 | 68.659 | 1.00 | 19.80 |
| ATOM | 1794 | CD | ARG | 241 | 17.266 | 19.307 | 69.474 | 1.00 | 21.32 |
| ATOM | 1795 | NE | ARG | 241 | 17.484 | 18.087 | 68.692 | 1.00 | 22.98 |
| ATOM | 1796 | CZ | ARG | 241 | 18.334 | 17.979 | 67.675 | 1.00 | 24.19 |
| ATOM | 1797 | NH1 | ARG | 241 | 19.068 | 19.018 | 67.302 | 1.00 | 25.28 |
| ATOM | 1798 | NH2 | ARG | 241 | 18.444 | 16.830 | 67.018 | 1.00 | 24.38 |
| ATOM | 1799 | C | ARG | 241 | 14.964 | 23.806 | 69.427 | 1.00 | 20.32 |
| ATOM | 1800 | O | ARG | 241 | 13.727 | 23.803 | 69.520 | 1.00 | 19.66 |
| ATOM | 1801 | N | ALA | 242 | 15.730 | 24.707 | 70.034 | 1.00 | 19.52 |
| ATOM | 1802 | CA | ALA | 242 | 15.133 | 25.771 | 70.832 | 1.00 | 19.63 |
| ATOM | 1803 | CB | ALA | 242 | 16.227 | 26.580 | 71.524 | 1.00 | 20.26 |
| ATOM | 1804 | C | ALA | 242 | 14.312 | 26.662 | 69.896 | 1.00 | 19.55 |
| ATOM | 1805 | O | ALA | 242 | 13.233 | 27.133 | 70.267 | 1.00 | 21.30 |
| ATOM | 1806 | N | ALA | 243 | 14.804 | 26.874 | 68.676 | 1.00 | 18.14 |
| ATOM | 1807 | CA | ALA | 243 | 14.074 | 27.700 | 67.707 | 1.00 | 19.54 |
| ATOM | 1808 | CB | ALA | 243 | 14.928 | 27.944 | 66.457 | 1.00 | 18.09 |
| ATOM | 1809 | C | ALA | 243 | 12.748 | 27.031 | 67.326 | 1.00 | 19.51 |
| ATOM | 1810 | O | ALA | 243 | 11.731 | 27.701 | 67.180 | 1.00 | 20.19 |
| ATOM | 1811 | N | VAL | 244 | 12.769 | 25.710 | 67.157 | 1.00 | 21.10 |
| ATOM | 1812 | CA | VAL | 244 | 11.554 | 24.969 | 66.818 | 1.00 | 19.17 |
| ATOM | 1813 | CB | VAL | 244 | 11.842 | 23.457 | 66.632 | 1.00 | 18.81 |
| ATOM | 1814 | CG1 | VAL | 244 | 10.536 | 22.668 | 66.538 | 1.00 | 18.20 |
| ATOM | 1815 | CG2 | VAL | 244 | 12.671 | 23.245 | 65.361 | 1.00 | 17.47 |
| ATOM | 1816 | C | VAL | 244 | 10.521 | 25.146 | 67.927 | 1.00 | 20.06 |
| ATOM | 1817 | O | VAL | 244 | 9.336 | 25.348 | 67.660 | 1.00 | 19.30 |
| ATOM | 1818 | N | ARG | 245 | 10.972 | 25.073 | 69.174 | 1.00 | 21.06 |
| ATOM | 1819 | CA | ARG | 245 | 10.063 | 25.227 | 70.297 | 1.00 | 21.32 |
| ATOM | 1820 | CB | ARG | 245 | 10.780 | 24.907 | 71.615 | 1.00 | 21.31 |
| ATOM | 1821 | CG | ARG | 245 | 11.128 | 23.427 | 71.766 | 1.00 | 22.58 |
| ATOM | 1822 | CD | ARG | 245 | 11.472 | 23.053 | 73.209 | 1.00 | 25.04 |
| ATOM | 1823 | NE | ARG | 245 | 12.697 | 23.674 | 73.698 | 1.00 | 26.83 |
| ATOM | 1824 | CZ | ARG | 245 | 13.930 | 23.291 | 73.375 | 1.00 | 29.75 |
| ATOM | 1825 | NH1 | ARG | 245 | 14.126 | 22.274 | 72.547 | 1.00 | 30.29 |
| ATOM | 1826 | NH2 | ARG | 245 | 14.980 | 23.921 | 73.896 | 1.00 | 29.20 |
| ATOM | 1827 | C | ARG | 245 | 9.435 | 26.619 | 70.352 | 1.00 | 21.20 |
| ATOM | 1828 | O | ARG | 245 | 8.236 | 26.749 | 70.605 | 1.00 | 21.04 |
| ATOM | 1829 | N | GLN | 246 | 10.231 | 27.653 | 70.095 | 1.00 | 22.00 |
| ATOM | 1830 | CA | GLN | 246 | 9.730 | 29.024 | 70.122 | 1.00 | 23.64 |
| ATOM | 1831 | CB | GLN | 246 | 10.877 | 30.012 | 69.919 | 1.00 | 26.15 |
| ATOM | 1832 | CG | GLN | 246 | 10.464 | 31.464 | 70.076 | 1.00 | 29.62 |
| ATOM | 1833 | CD | GLN | 246 | 11.652 | 32.399 | 70.056 | 1.00 | 33.46 |
| ATOM | 1834 | OE1 | GLN | 246 | 12.600 | 32.222 | 70.822 | 1.00 | 35.90 |
| ATOM | 1835 | NE2 | GLN | 246 | 11.609 | 33.404 | 69.185 | 1.00 | 34.94 |
| ATOM | 1836 | C | GLN | 246 | 8.683 | 29.220 | 69.036 | 1.00 | 24.20 |
| ATOM | 1837 | O | GLN | 246 | 7.664 | 29.880 | 69.245 | 1.00 | 23.85 |
| ATOM | 1838 | N | TYR | 247 | 8.942 | 28.651 | 67.864 | 1.00 | 21.90 |
| ATOM | 1839 | CA | TYR | 247 | 7.999 | 28.751 | 66.761 | 1.00 | 21.66 |
| ATOM | 1840 | CB | TYR | 247 | 8.571 | 28.037 | 65.528 | 1.00 | 19.75 |
| ATOM | 1841 | CG | TYR | 247 | 7.579 | 27.795 | 64.419 | 1.00 | 20.83 |
| ATOM | 1842 | CD1 | TYR | 247 | 6.815 | 28.837 | 63.899 | 1.00 | 19.45 |
| ATOM | 1843 | CE1 | TYR | 247 | 5.892 | 28.612 | 62.885 | 1.00 | 21.16 |
| ATOM | 1844 | CD2 | TYR | 247 | 7.398 | 26.517 | 63.891 | 1.00 | 21.25 |
| ATOM | 1845 | CE2 | TYR | 247 | 6.480 | 26.281 | 62.876 | 1.00 | 21.03 |
| ATOM | 1846 | CZ | TYR | 247 | 5.729 | 27.327 | 62.378 | 1.00 | 20.70 |
| ATOM | 1847 | OH | TYR | 247 | 4.802 | 27.085 | 61.388 | 1.00 | 20.47 |
| ATOM | 1848 | C | TYR | 247 | 6.673 | 28.125 | 67.199 | 1.00 | 21.28 |
| ATOM | 1849 | O | TYR | 247 | 5.609 | 28.723 | 67.026 | 1.00 | 21.42 |
| ATOM | 1850 | N | MET | 248 | 6.746 | 26.929 | 67.782 | 1.00 | 20.26 |
| ATOM | 1851 | CA | MET | 248 | 5.556 | 26.219 | 68.258 | 1.00 | 21.53 |
| ATOM | 1852 | CB | MET | 248 | 5.951 | 24.872 | 68.884 | 1.00 | 21.82 |
| ATOM | 1853 | CG | MET | 248 | 6.426 | 23.815 | 67.882 | 1.00 | 21.90 |
| ATOM | 1854 | SD | MET | 248 | 7.248 | 22.415 | 68.688 | 1.00 | 26.67 |
| ATOM | 1855 | CE | MET | 248 | 5.858 | 21.537 | 69.370 | 1.00 | 24.87 |
| ATOM | 1856 | C | MET | 248 | 4.786 | 27.040 | 69.293 | 1.00 | 22.86 |
| ATOM | 1857 | O | MET | 248 | 3.554 | 27.155 | 69.231 | 1.00 | 21.83 |
| ATOM | 1858 | N | ALA | 249 | 5.518 | 27.606 | 70.247 | 1.00 | 21.40 |
| ATOM | 1859 | CA | ALA | 249 | 4.898 | 28.405 | 71.299 | 1.00 | 21.90 |
| ATOM | 1860 | CB | ALA | 249 | 5.926 | 28.738 | 72.369 | 1.00 | 21.23 |
| ATOM | 1861 | C | ALA | 249 | 4.252 | 29.687 | 70.778 | 1.00 | 21.43 |
| ATOM | 1862 | O | ALA | 249 | 3.099 | 29.979 | 71.105 | 1.00 | 22.72 |
| ATOM | 1863 | N | GLU | 250 | 4.989 | 30.448 | 69.970 | 1.00 | 22.09 |
| ATOM | 1864 | CA | GLU | 250 | 4.469 | 31.707 | 69.433 | 1.00 | 23.27 |
| ATOM | 1865 | CB | GLU | 250 | 5.549 | 32.454 | 68.649 | 1.00 | 22.04 |
| ATOM | 1866 | CG | GLU | 250 | 6.815 | 32.693 | 69.442 | 1.00 | 24.15 |
| ATOM | 1867 | CD | GLU | 250 | 7.629 | 33.860 | 68.923 | 1.00 | 24.35 |
| ATOM | 1868 | OE1 | GLU | 250 | 7.549 | 34.169 | 67.719 | 1.00 | 25.60 |
| ATOM | 1869 | OE2 | GLU | 250 | 8.362 | 34.464 | 69.729 | 1.00 | 25.39 |
| ATOM | 1870 | C | GLU | 250 | 3.231 | 31.538 | 68.558 | 1.00 | 25.21 |
| ATOM | 1871 | O | GLU | 250 | 2.406 | 32.454 | 68.449 | 1.00 | 24.11 |
| ATOM | 1872 | N | VAL | 251 | 3.093 | 30.376 | 67.930 | 1.00 | 24.87 |
| ATOM | 1873 | CA | VAL | 251 | 1.927 | 30.135 | 67.094 | 1.00 | 24.81 |
| ATOM | 1874 | CB | VAL | 251 | 2.114 | 28.886 | 66.198 | 1.00 | 23.31 |
| ATOM | 1875 | CG1 | VAL | 251 | 0.827 | 28.605 | 65.431 | 1.00 | 22.66 |
| ATOM | 1876 | CG2 | VAL | 251 | 3.260 | 29.108 | 65.234 | 1.00 | 23.33 |
| ATOM | 1877 | C | VAL | 251 | 0.693 | 29.927 | 67.970 | 1.00 | 25.72 |
| ATOM | 1878 | O | VAL | 251 | −0.355 | 30.524 | 67.731 | 1.00 | 26.34 |
| ATOM | 1879 | N | GLU | 252 | 0.825 | 29.085 | 68.990 | 1.00 | 25.88 |
| ATOM | 1880 | CA | GLU | 252 | −0.291 | 28.804 | 69.885 | 1.00 | 28.97 |
| ATOM | 1881 | CB | GLU | 252 | 0.066 | 27.667 | 70.842 | 1.00 | 31.63 |
| ATOM | 1882 | CG | GLU | 252 | −1.076 | 27.268 | 71.762 | 1.00 | 35.49 |
| ATOM | 1883 | CD | GLU | 252 | −0.739 | 26.078 | 72.633 | 1.00 | 38.81 |
| ATOM | 1884 | OE1 | GLU | 252 | −1.557 | 25.730 | 73.513 | 1.00 | 41.32 |
| ATOM | 1885 | OE2 | GLU | 252 | 0.343 | 25.486 | 72.438 | 1.00 | 40.57 |
| ATOM | 1886 | C | GLU | 252 | −0.713 | 30.026 | 70.686 | 1.00 | 29.30 |
| ATOM | 1887 | O | GLU | 252 | −1.872 | 30.142 | 71.082 | 1.00 | 28.02 |
| ATOM | 1888 | N | SER | 253 | 0.233 | 30.930 | 70.922 | 1.00 | 30.46 |
| ATOM | 1889 | CA | SER | 253 | −0.038 | 32.148 | 71.681 | 1.00 | 32.50 |
| ATOM | 1890 | CB | SER | 253 | 1.209 | 32.572 | 72.450 | 1.00 | 32.17 |
| ATOM | 1891 | OG | SER | 253 | 1.459 | 31.671 | 73.519 | 1.00 | 39.52 |
| ATOM | 1892 | C | SER | 253 | −0.497 | 33.302 | 70.794 | 1.00 | 31.92 |
| ATOM | 1893 | O | SER | 253 | −1.051 | 34.288 | 71.283 | 1.00 | 32.17 |
| ATOM | 1894 | N | GLY | 254 | −0.264 | 33.178 | 69.492 | 1.00 | 30.27 |
| ATOM | 1895 | CA | GLY | 254 | −0.665 | 34.227 | 68.571 | 1.00 | 27.52 |
| ATOM | 1896 | C | GLY | 254 | 0.429 | 35.249 | 68.330 | 1.00 | 26.04 |
| ATOM | 1897 | O | GLY | 254 | 0.259 | 36.178 | 67.541 | 1.00 | 28.43 |
| ATOM | 1898 | N | VAL | 255 | 1.556 | 35.083 | 69.013 | 1.00 | 25.10 |
| ATOM | 1899 | CA | VAL | 255 | 2.678 | 35.998 | 68.861 | 1.00 | 24.43 |
| ATOM | 1900 | CB | VAL | 255 | 3.806 | 35.637 | 69.836 | 1.00 | 24.55 |
| ATOM | 1901 | CG1 | VAL | 255 | 5.016 | 36.530 | 69.601 | 1.00 | 25.47 |
| ATOM | 1902 | CG2 | VAL | 255 | 3.299 | 35.782 | 71.264 | 1.00 | 27.69 |
| ATOM | 1903 | C | VAL | 255 | 3.209 | 35.966 | 67.433 | 1.00 | 23.67 |
| ATOM | 1904 | O | VAL | 255 | 3.667 | 36.981 | 66.901 | 1.00 | 21.95 |
| ATOM | 1905 | N | TYR | 256 | 3.155 | 34.786 | 66.821 | 1.00 | 24.22 |
| ATOM | 1906 | CA | TYR | 256 | 3.597 | 34.614 | 65.441 | 1.00 | 22.81 |
| ATOM | 1907 | CB | TYR | 256 | 4.856 | 33.739 | 65.356 | 1.00 | 22.52 |
| ATOM | 1908 | CG | TYR | 256 | 5.361 | 33.602 | 63.935 | 1.00 | 22.38 |
| ATOM | 1909 | CD1 | TYR | 256 | 6.061 | 34.644 | 63.320 | 1.00 | 22.41 |
| ATOM | 1910 | CE1 | TYR | 256 | 6.436 | 34.574 | 61.975 | 1.00 | 23.46 |
| ATOM | 1911 | CD2 | TYR | 256 | 5.053 | 32.473 | 63.171 | 1.00 | 23.14 |
| ATOM | 1912 | CE2 | TYR | 256 | 5.419 | 32.388 | 61.825 | 1.00 | 22.31 |
| ATOM | 1913 | CZ | TYR | 256 | 6.108 | 33.443 | 61.234 | 1.00 | 25.45 |
| ATOM | 1914 | OH | TYR | 256 | 6.448 | 33.378 | 59.894 | 1.00 | 23.06 |
| ATOM | 1915 | C | TYR | 256 | 2.476 | 33.950 | 64.645 | 1.00 | 22.89 |
| ATOM | 1916 | O | TYR | 256 | 1.860 | 32.995 | 65.107 | 1.00 | 20.80 |
| ATOM | 1917 | N | PRO | 257 | 2.188 | 34.465 | 63.441 | 1.00 | 23.79 |
| ATOM | 1918 | CD | PRO | 257 | 1.258 | 33.861 | 62.468 | 1.00 | 25.59 |
| ATOM | 1919 | CA | PRO | 257 | 2.886 | 35.611 | 62.854 | 1.00 | 25.56 |
| ATOM | 1920 | CB | PRO | 257 | 2.485 | 35.542 | 61.379 | 1.00 | 26.50 |
| ATOM | 1921 | CG | PRO | 257 | 1.128 | 34.947 | 61.427 | 1.00 | 26.66 |
| ATOM | 1922 | C | PRO | 257 | 2.489 | 36.934 | 63.513 | 1.00 | 26.91 |
| ATOM | 1923 | O | PRO | 257 | 1.382 | 37.074 | 64.027 | 1.00 | 26.07 |
| ATOM | 1924 | N | GLY | 258 | 3.412 | 37.889 | 63.501 | 1.00 | 28.06 |
| ATOM | 1925 | CA | GLY | 258 | 3.141 | 39.182 | 64.092 | 1.00 | 31.45 |
| ATOM | 1926 | C | GLY | 258 | 2.550 | 40.108 | 63.052 | 1.00 | 33.08 |
| ATOM | 1927 | O | GLY | 258 | 2.454 | 39.750 | 61.875 | 1.00 | 31.82 |
| ATOM | 1928 | N | GLU | 259 | 2.153 | 41.301 | 63.476 | 1.00 | 33.88 |
| ATOM | 1929 | CA | GLU | 259 | 1.571 | 42.259 | 62.554 | 1.00 | 35.72 |
| ATOM | 1930 | CB | GLU | 259 | 1.118 | 43.513 | 63.304 | 1.00 | 37.56 |
| ATOM | 1931 | CG | GLU | 259 | 0.153 | 44.376 | 62.519 | 1.00 | 40.03 |
| ATOM | 1932 | CD | GLU | 259 | −1.186 | 43.695 | 62.296 | 1.00 | 42.11 |
| ATOM | 1933 | OE1 | GLU | 259 | −2.009 | 44.237 | 61.534 | 1.00 | 45.27 |
| ATOM | 1934 | OE2 | GLU | 259 | −1.421 | 42.620 | 62.886 | 1.00 | 44.73 |
| ATOM | 1935 | C | GLU | 259 | 2.606 | 42.629 | 61.498 | 1.00 | 36.27 |
| ATOM | 1936 | O | GLU | 259 | 2.258 | 43.046 | 60.391 | 1.00 | 38.03 |
| ATOM | 1937 | N | GLU | 260 | 3.881 | 42.468 | 61.844 | 1.00 | 36.02 |
| ATOM | 1938 | CA | GLU | 260 | 4.976 | 42.780 | 60.930 | 1.00 | 36.42 |
| ATOM | 1939 | CB | GLU | 260 | 6.318 | 42.804 | 61.673 | 1.00 | 39.45 |
| ATOM | 1940 | CG | GLU | 260 | 6.264 | 43.354 | 63.087 | 1.00 | 43.52 |
| ATOM | 1941 | CD | GLU | 260 | 5.723 | 42.347 | 64.088 | 1.00 | 46.00 |
| ATOM | 1942 | OE1 | GLU | 260 | 6.384 | 41.306 | 64.306 | 1.00 | 47.00 |
| ATOM | 1943 | OE2 | GLU | 260 | 4.637 | 42.596 | 64.656 | 1.00 | 46.44 |
| ATOM | 1944 | C | GLU | 260 | 5.057 | 41.724 | 59.836 | 1.00 | 35.35 |
| ATOM | 1945 | O | GLU | 260 | 5.677 | 41.940 | 58.797 | 1.00 | 34.80 |
| ATOM | 1946 | N | HIS | 261 | 4.434 | 40.577 | 60.084 | 1.00 | 34.70 |
| ATOM | 1947 | CA | HIS | 261 | 4.448 | 39.471 | 59.132 | 1.00 | 34.15 |
| ATOM | 1948 | CB | HIS | 261 | 4.760 | 38.164 | 59.857 | 1.00 | 32.10 |
| ATOM | 1949 | CG | HIS | 261 | 6.004 | 38.213 | 60.687 | 1.00 | 31.51 |
| ATOM | 1950 | CD2 | HIS | 261 | 6.200 | 38.019 | 62.013 | 1.00 | 29.70 |
| ATOM | 1951 | ND1 | HIS | 261 | 7.249 | 38.460 | 60.149 | 1.00 | 30.98 |
| ATOM | 1952 | CE1 | HIS | 261 | 8.158 | 38.411 | 61.106 | 1.00 | 28.80 |
| ATOM | 1953 | NE2 | HIS | 261 | 7.548 | 38.145 | 62.246 | 1.00 | 28.77 |
| ATOM | 1954 | C | HIS | 261 | 3.110 | 39.330 | 58.424 | 1.00 | 35.41 |
| ATOM | 1955 | O | HIS | 261 | 2.912 | 38.408 | 57.631 | 1.00 | 35.55 |
| ATOM | 1956 | N | SER | 262 | 2.195 | 40.248 | 58.716 | 1.00 | 35.97 |
| ATOM | 1957 | CA | SER | 262 | 0.864 | 40.226 | 58.133 | 1.00 | 36.99 |
| ATOM | 1958 | CB | SER | 262 | −0.173 | 40.483 | 59.227 | 1.00 | 35.31 |
| ATOM | 1959 | OG | SER | 262 | −0.052 | 39.540 | 60.279 | 1.00 | 37.80 |
| ATOM | 1960 | C | SER | 262 | 0.703 | 41.254 | 57.014 | 1.00 | 38.31 |
| ATOM | 1961 | O | SER | 262 | 1.483 | 42.203 | 56.909 | 1.00 | 37.66 |
| ATOM | 1962 | N | PHE | 263 | −0.312 | 41.050 | 56.178 | 1.00 | 40.01 |
| ATOM | 1963 | CA | PHE | 263 | −0.600 | 41.955 | 55.066 | 1.00 | 41.82 |
| ATOM | 1964 | CB | PHE | 263 | −0.434 | 41.243 | 53.718 | 1.00 | 42.58 |
| ATOM | 1965 | CG | PHE | 263 | 0.969 | 40.797 | 53.429 | 1.00 | 43.74 |
| ATOM | 1966 | CD1 | PHE | 263 | 1.306 | 39.447 | 53.467 | 1.00 | 43.83 |
| ATOM | 1967 | CD2 | PHE | 263 | 1.954 | 41.727 | 53.110 | 1.00 | 44.25 |
| ATOM | 1968 | CE1 | PHE | 263 | 2.605 | 39.029 | 53.190 | 1.00 | 44.88 |
| ATOM | 1969 | CE2 | PHE | 263 | 3.254 | 41.322 | 52.832 | 1.00 | 45.05 |
| ATOM | 1970 | CZ | PHE | 263 | 3.583 | 39.970 | 52.871 | 1.00 | 45.70 |
| ATOM | 1971 | C | PHE | 263 | −2.026 | 42.482 | 55.169 | 1.00 | 42.76 |
| ATOM | 1972 | O | PHE | 263 | −2.827 | 41.984 | 55.961 | 1.00 | 41.84 |
| ATOM | 1973 | N | HIS | 264 | −2.333 | 43.491 | 54.359 | 1.00 | 44.75 |
| ATOM | 1974 | CA | HIS | 264 | −3.661 | 44.102 | 54.336 | 1.00 | 47.39 |
| ATOM | 1975 | CB | HIS | 264 | −3.719 | 45.285 | 55.303 | 1.00 | 48.50 |
| ATOM | 1976 | CG | HIS | 264 | −3.536 | 44.897 | 56.735 | 1.00 | 49.55 |
| ATOM | 1977 | CD2 | HIS | 264 | −2.580 | 45.229 | 57.635 | 1.00 | 49.70 |
| ATOM | 1978 | ND1 | HIS | 264 | −4.394 | 44.037 | 57.386 | 1.00 | 49.75 |
| ATOM | 1979 | CE1 | HIS | 264 | −3.973 | 43.854 | 58.625 | 1.00 | 50.24 |
| ATOM | 1980 | NE2 | HIS | 264 | −2.874 | 44.565 | 58.801 | 1.00 | 49.74 |
| ATOM | 1981 | C | HIS | 264 | −4.020 | 44.576 | 52.931 | 1.00 | 47.60 |
| ATOM | 1982 | O | HIS | 264 | −5.144 | 44.275 | 52.483 | 1.00 | 48.91 |
| ATOM | 1983 | OXT | HIS | 264 | −3.178 | 45.249 | 52.302 | 1.00 | 48.36 |
| ATOM | 1984 | C1 | KPL | 265 | 5.087 | 27.716 | 51.358 | 1.00 | 41.50 |
| ATOM | 1985 | C2 | KPL | 265 | 4.190 | 26.479 | 51.578 | 1.00 | 40.60 |
| ATOM | 1986 | C3 | KPL | 265 | 4.654 | 25.755 | 52.846 | 1.00 | 39.94 |
| ATOM | 1987 | C4 | KPL | 265 | 2.727 | 26.938 | 51.779 | 1.00 | 41.78 |
| ATOM | 1988 | O1 | KPL | 265 | 2.243 | 27.630 | 50.619 | 1.00 | 43.47 |
| ATOM | 1989 | C5 | KPL | 265 | 4.309 | 25.525 | 50.360 | 1.00 | 40.08 |
| ATOM | 1990 | O2 | KPL | 265 | 3.322 | 25.239 | 49.713 | 1.00 | 38.16 |
| ATOM | 1991 | C6 | KPL | 265 | 5.636 | 24.923 | 49.944 | 1.00 | 39.07 |
| ATOM | 1992 | O3 | KPL | 265 | 6.653 | 25.170 | 50.562 | 1.00 | 40.08 |
| ATOM | 1993 | O4 | KPL | 265 | 5.695 | 24.104 | 48.874 | 1.00 | 38.37 |
| ATOM | 1994 | CB | MET | 301 | 16.154 | 43.498 | 31.231 | 1.00 | 80.41 |
| ATOM | 1995 | CG | MET | 301 | 15.177 | 44.253 | 30.325 | 1.00 | 81.85 |
| ATOM | 1996 | SD | MET | 301 | 13.933 | 45.244 | 31.185 | 1.00 | 84.34 |
| ATOM | 1997 | CE | MET | 301 | 12.458 | 44.235 | 30.946 | 1.00 | 83.29 |
| ATOM | 1998 | C | MET | 301 | 14.844 | 42.880 | 33.290 | 1.00 | 78.13 |
| ATOM | 1999 | O | MET | 301 | 15.030 | 44.035 | 33.680 | 1.00 | 78.18 |
| ATOM | 2000 | N | MET | 301 | 14.641 | 41.532 | 31.184 | 1.00 | 78.62 |
| ATOM | 2001 | CA | MET | 301 | 15.549 | 42.343 | 32.045 | 1.00 | 78.92 |
| ATOM | 2002 | N | LYS | 302 | 14.042 | 42.021 | 33.914 | 1.00 | 76.63 |
| ATOM | 2003 | CA | LYS | 302 | 13.300 | 42.378 | 35.118 | 1.00 | 75.10 |
| ATOM | 2004 | CB | LYS | 302 | 11.941 | 42.982 | 34.740 | 1.00 | 75.44 |
| ATOM | 2005 | CG | LYS | 302 | 11.997 | 44.455 | 34.355 | 1.00 | 75.76 |
| ATOM | 2006 | CD | LYS | 302 | 12.271 | 45.330 | 35.571 | 1.00 | 75.67 |
| ATOM | 2007 | CE | LYS | 302 | 11.149 | 45.212 | 36.594 | 1.00 | 75.44 |
| ATOM | 2008 | NZ | LYS | 302 | 11.411 | 46.017 | 37.817 | 1.00 | 75.82 |
| ATOM | 2009 | C | LYS | 302 | 13.091 | 41.203 | 36.082 | 1.00 | 73.54 |
| ATOM | 2010 | O | LYS | 302 | 13.159 | 41.380 | 37.300 | 1.00 | 74.12 |
| ATOM | 2011 | N | PRO | 303 | 12.847 | 39.987 | 35.554 | 1.00 | 71.41 |
| ATOM | 2012 | CD | PRO | 303 | 12.659 | 38.812 | 36.425 | 1.00 | 70.80 |
| ATOM | 2013 | CA | PRO | 303 | 12.743 | 39.596 | 34.143 | 1.00 | 69.04 |
| ATOM | 2014 | CB | PRO | 303 | 12.752 | 38.074 | 34.213 | 1.00 | 69.91 |
| ATOM | 2015 | CG | PRO | 303 | 12.025 | 37.812 | 35.486 | 1.00 | 70.53 |
| ATOM | 2016 | C | PRO | 303 | 11.499 | 40.136 | 33.444 | 1.00 | 66.77 |
| ATOM | 2017 | O | PRO | 303 | 10.485 | 40.417 | 34.083 | 1.00 | 66.86 |
| ATOM | 2018 | N | THR | 304 | 11.588 | 40.276 | 32.127 | 1.00 | 64.02 |
| ATOM | 2019 | CA | THR | 304 | 10.474 | 40.781 | 31.337 | 1.00 | 61.28 |
| ATOM | 2020 | CB | THR | 304 | 10.830 | 40.818 | 29.842 | 1.00 | 61.21 |
| ATOM | 2021 | OG1 | THR | 304 | 12.085 | 41.486 | 29.668 | 1.00 | 60.91 |
| ATOM | 2022 | CG2 | THR | 304 | 9.756 | 41.560 | 29.062 | 1.00 | 60.88 |
| ATOM | 2023 | C | THR | 304 | 9.247 | 39.897 | 31.525 | 1.00 | 59.60 |
| ATOM | 2024 | O | THR | 304 | 9.357 | 38.673 | 31.563 | 1.00 | 59.05 |
| ATOM | 2025 | N | THR | 305 | 8.079 | 40.522 | 31.644 | 1.00 | 57.62 |
| ATOM | 2026 | CA | THR | 305 | 6.836 | 39.783 | 31.829 | 1.00 | 55.83 |
| ATOM | 2027 | CB | THR | 305 | 6.207 | 40.074 | 33.204 | 1.00 | 55.66 |
| ATOM | 2028 | OG1 | THR | 305 | 5.873 | 41.465 | 33.293 | 1.00 | 55.27 |
| ATOM | 2029 | CG2 | THR | 305 | 7.176 | 39.713 | 34.317 | 1.00 | 55.55 |
| ATOM | 2030 | C | THR | 305 | 5.810 | 40.133 | 30.758 | 1.00 | 55.15 |
| ATOM | 2031 | O | THR | 305 | 5.968 | 41.108 | 30.020 | 1.00 | 54.85 |
| ATOM | 2032 | N | ILE | 306 | 4.756 | 39.328 | 30.680 | 1.00 | 54.30 |
| ATOM | 2033 | CA | ILE | 306 | 3.698 | 39.543 | 29.704 | 1.00 | 54.29 |
| ATOM | 2034 | CB | ILE | 306 | 2.606 | 38.462 | 29.821 | 1.00 | 54.32 |
| ATOM | 2035 | CG2 | ILE | 306 | 1.645 | 38.567 | 28.644 | 1.00 | 54.60 |
| ATOM | 2036 | CG1 | ILE | 306 | 3.249 | 37.074 | 29.846 | 1.00 | 54.90 |
| ATOM | 2037 | CD1 | ILE | 306 | 2.276 | 35.949 | 30.142 | 1.00 | 55.32 |
| ATOM | 2038 | C | ILE | 306 | 3.061 | 40.905 | 29.940 | 1.00 | 54.16 |
| ATOM | 2039 | O | ILE | 306 | 2.648 | 41.584 | 28.999 | 1.00 | 53.93 |
| ATOM | 2040 | N | SER | 307 | 2.990 | 41.299 | 31.208 | 1.00 | 53.96 |
| ATOM | 2041 | CA | SER | 307 | 2.402 | 42.579 | 31.588 | 1.00 | 54.22 |
| ATOM | 2042 | CB | SER | 307 | 2.523 | 42.777 | 33.101 | 1.00 | 54.20 |
| ATOM | 2043 | OG | SER | 307 | 1.851 | 41.744 | 33.801 | 1.00 | 55.29 |
| ATOM | 2044 | C | SER | 307 | 3.064 | 43.745 | 30.855 | 1.00 | 53.77 |
| ATOM | 2045 | O | SER | 307 | 2.383 | 44.653 | 30.379 | 1.00 | 54.19 |
| ATOM | 2046 | N | LEU | 308 | 4.392 | 43.712 | 30.770 | 1.00 | 53.02 |
| ATOM | 2047 | CA | LEU | 308 | 5.142 | 44.761 | 30.092 | 1.00 | 53.53 |
| ATOM | 2048 | CB | LEU | 308 | 6.630 | 44.411 | 30.049 | 1.00 | 53.40 |
| ATOM | 2049 | CG | LEU | 308 | 7.434 | 44.783 | 31.295 | 1.00 | 54.50 |
| ATOM | 2050 | CD1 | LEU | 308 | 8.837 | 44.205 | 31.202 | 1.00 | 54.63 |
| ATOM | 2051 | CD2 | LEU | 308 | 7.487 | 46.304 | 31.424 | 1.00 | 54.20 |
| ATOM | 2052 | C | LEU | 308 | 4.631 | 44.988 | 28.676 | 1.00 | 53.65 |
| ATOM | 2053 | O | LEU | 308 | 4.355 | 46.120 | 28.277 | 1.00 | 53.25 |
| ATOM | 2054 | N | LEU | 309 | 4.509 | 43.905 | 27.917 | 1.00 | 53.27 |
| ATOM | 2055 | CA | LEU | 309 | 4.024 | 43.990 | 26.549 | 1.00 | 53.60 |
| ATOM | 2056 | CB | LEU | 309 | 3.994 | 42.599 | 25.914 | 1.00 | 53.39 |
| ATOM | 2057 | CG | LEU | 309 | 5.336 | 41.872 | 25.803 | 1.00 | 53.15 |
| ATOM | 2058 | CD1 | LEU | 309 | 5.108 | 40.469 | 25.271 | 1.00 | 52.80 |
| ATOM | 2059 | CD2 | LEU | 309 | 6.272 | 42.646 | 24.890 | 1.00 | 52.50 |
| ATOM | 2060 | C | LEU | 309 | 2.625 | 44.598 | 26.530 | 1.00 | 53.99 |
| ATOM | 2061 | O | LEU | 309 | 2.312 | 45.429 | 25.677 | 1.00 | 53.60 |
| ATOM | 2062 | N | GLN | 310 | 1.790 | 44.182 | 27.479 | 1.00 | 54.06 |
| ATOM | 2063 | CA | GLN | 310 | 0.425 | 44.685 | 27.576 | 1.00 | 55.21 |
| ATOM | 2064 | CB | GLN | 310 | −0.319 | 43.979 | 28.716 | 1.00 | 55.07 |
| ATOM | 2065 | CG | GLN | 310 | −1.810 | 44.283 | 28.790 | 1.00 | 55.61 |
| ATOM | 2066 | CD | GLN | 310 | −2.577 | 43.772 | 27.581 | 1.00 | 56.41 |
| ATOM | 2067 | OE1 | GLN | 310 | −2.395 | 44.254 | 26.463 | 1.00 | 56.40 |
| ATOM | 2068 | NE2 | GLN | 310 | −3.438 | 42.784 | 27.803 | 1.00 | 57.08 |
| ATOM | 2069 | C | GLN | 310 | 0.441 | 46.192 | 27.824 | 1.00 | 56.02 |
| ATOM | 2070 | O | GLN | 310 | −0.263 | 46.949 | 27.153 | 1.00 | 56.00 |
| ATOM | 2071 | N | LYS | 311 | 1.252 | 46.620 | 28.786 | 1.00 | 56.45 |
| ATOM | 2072 | CA | LYS | 311 | 1.366 | 48.036 | 29.121 | 1.00 | 57.85 |
| ATOM | 2073 | CB | LYS | 311 | 2.361 | 48.236 | 30.266 | 1.00 | 58.44 |
| ATOM | 2074 | CG | LYS | 311 | 2.419 | 49.668 | 30.777 | 1.00 | 59.71 |
| ATOM | 2075 | CD | LYS | 311 | 3.851 | 50.156 | 30.939 | 1.00 | 60.00 |
| ATOM | 2076 | CE | LYS | 311 | 4.611 | 49.364 | 31.989 | 1.00 | 60.14 |
| ATOM | 2077 | NZ | LYS | 311 | 6.013 | 49.849 | 32.114 | 1.00 | 59.97 |
| ATOM | 2078 | C | LYS | 311 | 1.829 | 48.836 | 27.906 | 1.00 | 57.84 |
| ATOM | 2079 | O | LYS | 311 | 1.341 | 49.938 | 27.654 | 1.00 | 57.02 |
| ATOM | 2080 | N | TYR | 312 | 2.774 | 48.269 | 27.160 | 1.00 | 58.58 |
| ATOM | 2081 | CA | TYR | 312 | 3.316 | 48.913 | 25.970 | 1.00 | 59.79 |
| ATOM | 2082 | CB | TYR | 312 | 4.369 | 48.014 | 25.314 | 1.00 | 60.90 |
| ATOM | 2083 | CG | TYR | 312 | 5.642 | 47.839 | 26.119 | 1.00 | 62.17 |
| ATOM | 2084 | CD1 | TYR | 312 | 6.639 | 46.960 | 25.695 | 1.00 | 62.63 |
| ATOM | 2085 | CE1 | TYR | 312 | 7.818 | 46.799 | 26.424 | 1.00 | 63.39 |
| ATOM | 2086 | CD2 | TYR | 312 | 5.855 | 48.557 | 27.297 | 1.00 | 62.71 |
| ATOM | 2087 | CE2 | TYR | 312 | 7.030 | 48.404 | 28.033 | 1.00 | 63.56 |
| ATOM | 2088 | CZ | TYR | 312 | 8.006 | 47.523 | 27.590 | 1.00 | 63.51 |
| ATOM | 2089 | OH | TYR | 312 | 9.170 | 47.369 | 28.311 | 1.00 | 63.95 |
| ATOM | 2090 | C | TYR | 312 | 2.230 | 49.243 | 24.954 | 1.00 | 60.36 |
| ATOM | 2091 | O | TYR | 312 | 2.287 | 50.279 | 24.289 | 1.00 | 60.60 |
| ATOM | 2092 | N | LYS | 313 | 1.244 | 48.360 | 24.830 | 1.00 | 60.33 |
| ATOM | 2093 | CA | LYS | 313 | 0.155 | 48.579 | 23.887 | 1.00 | 60.59 |
| ATOM | 2094 | CB | LYS | 313 | −0.720 | 47.324 | 23.773 | 1.00 | 59.68 |
| ATOM | 2095 | CG | LYS | 313 | −1.855 | 47.466 | 22.766 | 1.00 | 59.36 |
| ATOM | 2096 | CD | LYS | 313 | −2.535 | 46.141 | 22.466 | 1.00 | 57.36 |
| ATOM | 2097 | CE | LYS | 313 | −3.587 | 46.318 | 21.377 | 1.00 | 57.43 |
| ATOM | 2098 | NZ | LYS | 313 | −4.149 | 45.027 | 20.902 | 1.00 | 57.01 |
| ATOM | 2099 | C | LYS | 313 | −0.689 | 49.771 | 24.330 | 1.00 | 60.98 |
| ATOM | 2100 | O | LYS | 313 | −1.155 | 50.558 | 23.505 | 1.00 | 60.46 |
| ATOM | 2101 | N | GLN | 314 | −0.876 | 49.903 | 25.638 | 1.00 | 61.89 |
| ATOM | 2102 | CA | GLN | 314 | −1.656 | 51.004 | 26.186 | 1.00 | 63.46 |
| ATOM | 2103 | CB | GLN | 314 | −1.968 | 50.747 | 27.661 | 1.00 | 63.34 |
| ATOM | 2104 | CG | GLN | 314 | −2.812 | 49.507 | 27.898 | 1.00 | 64.06 |
| ATOM | 2105 | CD | GLN | 314 | −3.090 | 49.266 | 29.366 | 1.00 | 64.42 |
| ATOM | 2106 | OE1 | GLN | 314 | −3.675 | 50.108 | 30.047 | 1.00 | 64.71 |
| ATOM | 2107 | NE2 | GLN | 314 | −2.672 | 48.110 | 29.862 | 1.00 | 64.63 |
| ATOM | 2108 | C | GLN | 314 | −0.890 | 52.313 | 26.040 | 1.00 | 64.13 |
| ATOM | 2109 | O | GLN | 314 | −1.399 | 53.383 | 26.378 | 1.00 | 65.09 |
| ATOM | 2110 | N | GLU | 315 | 0.336 | 52.217 | 25.533 | 1.00 | 64.44 |
| ATOM | 2111 | CA | GLU | 315 | 1.186 | 53.386 | 25.332 | 1.00 | 64.89 |
| ATOM | 2112 | CB | GLU | 315 | 2.476 | 53.254 | 26.149 | 1.00 | 65.03 |
| ATOM | 2113 | CG | GLU | 315 | 2.250 | 53.017 | 27.632 | 1.00 | 65.70 |
| ATOM | 2114 | CD | GLU | 315 | 3.548 | 52.910 | 28.407 | 1.00 | 65.91 |
| ATOM | 2115 | OE1 | GLU | 315 | 4.414 | 52.101 | 28.013 | 1.00 | 66.19 |
| ATOM | 2116 | OE2 | GLU | 315 | 3.702 | 53.631 | 29.414 | 1.00 | 66.44 |
| ATOM | 2117 | C | GLU | 315 | 1.532 | 53.541 | 23.855 | 1.00 | 64.71 |
| ATOM | 2118 | O | GLU | 315 | 2.323 | 54.408 | 23.480 | 1.00 | 64.82 |
| ATOM | 2119 | N | LYS | 316 | 0.938 | 52.692 | 23.023 | 1.00 | 64.19 |
| ATOM | 2120 | CA | LYS | 316 | 1.176 | 52.731 | 21.585 | 1.00 | 64.21 |
| ATOM | 2121 | CB | LYS | 316 | 0.604 | 54.024 | 20.997 | 1.00 | 64.07 |
| ATOM | 2122 | CG | LYS | 316 | −0.840 | 54.306 | 21.387 | 1.00 | 64.10 |
| ATOM | 2123 | CD | LYS | 316 | −1.794 | 53.259 | 20.837 | 1.00 | 63.85 |
| ATOM | 2124 | CE | LYS | 316 | −3.220 | 53.514 | 21.306 | 1.00 | 63.79 |
| ATOM | 2125 | NZ | LYS | 316 | −3.703 | 54.874 | 20.934 | 1.00 | 63.74 |
| ATOM | 2126 | C | LYS | 316 | 2.671 | 52.643 | 21.276 | 1.00 | 64.35 |
| ATOM | 2127 | O | LYS | 316 | 3.157 | 53.260 | 20.327 | 1.00 | 64.34 |
| ATOM | 2128 | N | LYS | 317 | 3.395 | 51.876 | 22.087 | 1.00 | 64.27 |
| ATOM | 2129 | CA | LYS | 317 | 4.835 | 51.702 | 21.907 | 1.00 | 63.70 |
| ATOM | 2130 | CB | LYS | 317 | 5.553 | 51.810 | 23.257 | 1.00 | 64.31 |
| ATOM | 2131 | CG | LYS | 317 | 7.061 | 51.596 | 23.178 | 1.00 | 65.23 |
| ATOM | 2132 | CD | LYS | 317 | 7.689 | 51.451 | 24.560 | 1.00 | 65.71 |
| ATOM | 2133 | CE | LYS | 317 | 7.548 | 52.720 | 25.387 | 1.00 | 66.37 |
| ATOM | 2134 | NZ | LYS | 317 | 8.145 | 52.562 | 26.744 | 1.00 | 66.68 |
| ATOM | 2135 | C | LYS | 317 | 5.157 | 50.349 | 21.273 | 1.00 | 62.73 |
| ATOM | 2136 | O | LYS | 317 | 5.221 | 49.331 | 21.964 | 1.00 | 62.19 |
| ATOM | 2137 | N | ARG | 318 | 5.361 | 50.342 | 19.959 | 1.00 | 61.20 |
| ATOM | 2138 | CA | ARG | 318 | 5.682 | 49.109 | 19.250 | 1.00 | 59.98 |
| ATOM | 2139 | CB | ARG | 318 | 5.760 | 49.370 | 17.743 | 1.00 | 60.40 |
| ATOM | 2140 | CG | ARG | 318 | 4.416 | 49.244 | 17.040 | 1.00 | 61.29 |
| ATOM | 2141 | CD | ARG | 318 | 4.469 | 49.737 | 15.604 | 1.00 | 61.24 |
| ATOM | 2142 | NE | ARG | 318 | 4.450 | 51.196 | 15.528 | 1.00 | 62.30 |
| ATOM | 2143 | CZ | ARG | 318 | 4.415 | 51.885 | 14.392 | 1.00 | 62.88 |
| ATOM | 2144 | NH1 | ARG | 318 | 4.397 | 51.249 | 13.227 | 1.00 | 62.95 |
| ATOM | 2145 | NH2 | ARG | 318 | 4.391 | 53.211 | 14.421 | 1.00 | 62.95 |
| ATOM | 2146 | C | ARG | 318 | 6.990 | 48.507 | 19.754 | 1.00 | 58.84 |
| ATOM | 2147 | O | ARG | 318 | 8.024 | 49.174 | 19.780 | 1.00 | 58.27 |
| ATOM | 2148 | N | PHE | 319 | 6.928 | 47.241 | 20.156 | 1.00 | 57.39 |
| ATOM | 2149 | CA | PHE | 319 | 8.089 | 46.530 | 20.677 | 1.00 | 55.69 |
| ATOM | 2150 | CB | PHE | 319 | 7.725 | 45.856 | 22.005 | 1.00 | 56.21 |
| ATOM | 2151 | CG | PHE | 319 | 6.465 | 45.036 | 21.945 | 1.00 | 56.77 |
| ATOM | 2152 | CD1 | PHE | 319 | 6.465 | 43.768 | 21.368 | 1.00 | 56.74 |
| ATOM | 2153 | CD2 | PHE | 319 | 5.270 | 45.543 | 22.448 | 1.00 | 56.37 |
| ATOM | 2154 | CE1 | PHE | 319 | 5.291 | 43.018 | 21.294 | 1.00 | 57.23 |
| ATOM | 2155 | CE2 | PHE | 319 | 4.092 | 44.803 | 22.379 | 1.00 | 56.83 |
| ATOM | 2156 | CZ | PHE | 319 | 4.101 | 43.539 | 21.801 | 1.00 | 56.54 |
| ATOM | 2157 | C | PHE | 319 | 8.632 | 45.497 | 19.692 | 1.00 | 54.04 |
| ATOM | 2158 | O | PHE | 319 | 7.932 | 45.063 | 18.776 | 1.00 | 53.79 |
| ATOM | 2159 | N | ALA | 320 | 9.887 | 45.106 | 19.890 | 1.00 | 51.83 |
| ATOM | 2160 | CA | ALA | 320 | 10.527 | 44.135 | 19.013 | 1.00 | 49.46 |
| ATOM | 2161 | CB | ALA | 320 | 11.880 | 44.665 | 18.562 | 1.00 | 49.67 |
| ATOM | 2162 | C | ALA | 320 | 10.695 | 42.773 | 19.676 | 1.00 | 47.51 |
| ATOM | 2163 | O | ALA | 320 | 10.899 | 42.677 | 20.886 | 1.00 | 47.41 |
| ATOM | 2164 | N | THR | 321 | 10.607 | 41.721 | 18.869 | 1.00 | 45.20 |
| ATOM | 2165 | CA | THR | 321 | 10.755 | 40.355 | 19.357 | 1.00 | 43.60 |
| ATOM | 2166 | CB | THR | 321 | 9.383 | 39.692 | 19.579 | 1.00 | 43.59 |
| ATOM | 2167 | OG1 | THR | 321 | 8.618 | 40.472 | 20.506 | 1.00 | 44.19 |
| ATOM | 2168 | CG2 | THR | 321 | 9.553 | 38.293 | 20.132 | 1.00 | 44.04 |
| ATOM | 2169 | C | THR | 321 | 11.536 | 39.542 | 18.331 | 1.00 | 41.76 |
| ATOM | 2170 | O | THR | 321 | 11.456 | 39.804 | 17.130 | 1.00 | 41.89 |
| ATOM | 2171 | N | ILE | 322 | 12.290 | 38.551 | 18.798 | 1.00 | 40.55 |
| ATOM | 2172 | CA | ILE | 322 | 13.077 | 37.729 | 17.887 | 1.00 | 38.74 |
| ATOM | 2173 | CB | ILE | 322 | 14.479 | 38.338 | 17.688 | 1.00 | 39.69 |
| ATOM | 2174 | CG2 | ILE | 322 | 15.310 | 38.150 | 18.954 | 1.00 | 38.21 |
| ATOM | 2175 | CG1 | ILE | 322 | 15.160 | 37.692 | 16.479 | 1.00 | 39.62 |
| ATOM | 2176 | CD1 | ILE | 322 | 16.436 | 38.388 | 16.055 | 1.00 | 40.64 |
| ATOM | 2177 | C | ILE | 322 | 13.220 | 36.289 | 18.375 | 1.00 | 37.51 |
| ATOM | 2178 | O | ILE | 322 | 13.037 | 36.005 | 19.557 | 1.00 | 36.20 |
| ATOM | 2179 | N | THR | 323 | 13.544 | 35.381 | 17.458 | 1.00 | 35.23 |
| ATOM | 2180 | CA | THR | 323 | 13.718 | 33.980 | 17.817 | 1.00 | 33.67 |
| ATOM | 2181 | CB | THR | 323 | 13.381 | 33.034 | 16.640 | 1.00 | 33.56 |
| ATOM | 2182 | OG1 | THR | 323 | 14.346 | 33.202 | 15.596 | 1.00 | 33.89 |
| ATOM | 2183 | CG2 | THR | 323 | 11.996 | 33.331 | 16.096 | 1.00 | 34.18 |
| ATOM | 2184 | C | THR | 323 | 15.158 | 33.717 | 18.244 | 1.00 | 31.77 |
| ATOM | 2185 | O | THR | 323 | 16.071 | 34.459 | 17.879 | 1.00 | 30.93 |
| ATOM | 2186 | N | ALA | 324 | 15.345 | 32.661 | 19.033 | 1.00 | 30.73 |
| ATOM | 2187 | CA | ALA | 324 | 16.662 | 32.257 | 19.518 | 1.00 | 27.91 |
| ATOM | 2188 | CB | ALA | 324 | 17.022 | 33.025 | 20.783 | 1.00 | 29.81 |
| ATOM | 2189 | C | ALA | 324 | 16.618 | 30.758 | 19.800 | 1.00 | 28.38 |
| ATOM | 2190 | O | ALA | 324 | 15.618 | 30.247 | 20.312 | 1.00 | 26.39 |
| ATOM | 2191 | N | TYR | 325 | 17.703 | 30.059 | 19.472 | 1.00 | 27.03 |
| ATOM | 2192 | CA | TYR | 325 | 17.759 | 28.616 | 19.663 | 1.00 | 27.26 |
| ATOM | 2193 | CB | TYR | 325 | 17.603 | 27.909 | 18.315 | 1.00 | 25.23 |
| ATOM | 2194 | CG | TYR | 325 | 16.645 | 28.596 | 17.372 | 1.00 | 24.94 |
| ATOM | 2195 | CD1 | TYR | 325 | 17.109 | 29.501 | 16.417 | 1.00 | 23.93 |
| ATOM | 2196 | CE1 | TYR | 325 | 16.234 | 30.125 | 15.533 | 1.00 | 25.04 |
| ATOM | 2197 | CD2 | TYR | 325 | 15.275 | 28.336 | 17.427 | 1.00 | 23.23 |
| ATOM | 2198 | CE2 | TYR | 325 | 14.392 | 28.954 | 16.552 | 1.00 | 22.55 |
| ATOM | 2199 | CZ | TYR | 325 | 14.876 | 29.845 | 15.606 | 1.00 | 24.69 |
| ATOM | 2200 | OH | TYR | 325 | 14.003 | 30.434 | 14.723 | 1.00 | 25.39 |
| ATOM | 2201 | C | TYR | 325 | 19.038 | 28.131 | 20.333 | 1.00 | 27.49 |
| ATOM | 2202 | O | TYR | 325 | 19.287 | 26.931 | 20.400 | 1.00 | 28.39 |
| ATOM | 2203 | N | ASP | 326 | 19.854 | 29.052 | 20.827 | 1.00 | 27.48 |
| ATOM | 2204 | CA | ASP | 326 | 21.082 | 28.647 | 21.488 | 1.00 | 27.27 |
| ATOM | 2205 | CB | ASP | 326 | 22.182 | 28.392 | 20.453 | 1.00 | 26.92 |
| ATOM | 2206 | CG | ASP | 326 | 22.645 | 29.661 | 19.767 | 1.00 | 27.37 |
| ATOM | 2207 | OD1 | ASP | 326 | 23.394 | 30.439 | 20.397 | 1.00 | 28.68 |
| ATOM | 2208 | OD2 | ASP | 326 | 22.253 | 29.881 | 18.603 | 1.00 | 28.94 |
| ATOM | 2209 | C | ASP | 326 | 21.541 | 29.677 | 22.510 | 1.00 | 28.59 |
| ATOM | 2210 | O | ASP | 326 | 20.991 | 30.773 | 22.593 | 1.00 | 30.01 |
| ATOM | 2211 | N | TYR | 327 | 22.547 | 29.302 | 23.289 | 1.00 | 28.76 |
| ATOM | 2212 | CA | TYR | 327 | 23.106 | 30.152 | 24.332 | 1.00 | 30.47 |
| ATOM | 2213 | CB | TYR | 327 | 24.203 | 29.385 | 25.073 | 1.00 | 31.91 |
| ATOM | 2214 | CG | TYR | 327 | 24.997 | 30.215 | 26.057 | 1.00 | 35.27 |
| ATOM | 2215 | CD1 | TYR | 327 | 24.465 | 30.562 | 27.300 | 1.00 | 36.04 |
| ATOM | 2216 | CE1 | TYR | 327 | 25.196 | 31.331 | 28.209 | 1.00 | 37.29 |
| ATOM | 2217 | CD2 | TYR | 327 | 26.281 | 30.658 | 25.744 | 1.00 | 35.38 |
| ATOM | 2218 | CE2 | TYR | 327 | 27.018 | 31.427 | 26.643 | 1.00 | 36.93 |
| ATOM | 2219 | CZ | TYR | 327 | 26.472 | 31.759 | 27.869 | 1.00 | 37.24 |
| ATOM | 2220 | OH | TYR | 327 | 27.198 | 32.521 | 28.755 | 1.00 | 37.97 |
| ATOM | 2221 | C | TYR | 327 | 23.677 | 31.461 | 23.793 | 1.00 | 30.48 |
| ATOM | 2222 | O | TYR | 327 | 23.216 | 32.543 | 24.148 | 1.00 | 29.09 |
| ATOM | 2223 | N | SER | 328 | 24.685 | 31.347 | 22.935 | 1.00 | 31.30 |
| ATOM | 2224 | CA | SER | 328 | 25.350 | 32.509 | 22.364 | 1.00 | 31.80 |
| ATOM | 2225 | CB | SER | 328 | 26.252 | 32.077 | 21.208 | 1.00 | 31.10 |
| ATOM | 2226 | OG | SER | 328 | 27.287 | 31.231 | 21.685 | 1.00 | 31.34 |
| ATOM | 2227 | C | SER | 328 | 24.411 | 33.620 | 21.905 | 1.00 | 33.46 |
| ATOM | 2228 | O | SER | 328 | 24.409 | 34.712 | 22.478 | 1.00 | 35.08 |
| ATOM | 2229 | N | PHE | 329 | 23.608 | 33.353 | 20.882 | 1.00 | 33.75 |
| ATOM | 2230 | CA | PHE | 329 | 22.695 | 34.373 | 20.380 | 1.00 | 35.28 |
| ATOM | 2231 | CB | PHE | 329 | 21.957 | 33.876 | 19.134 | 1.00 | 36.14 |
| ATOM | 2232 | CG | PHE | 329 | 22.794 | 33.915 | 17.884 | 1.00 | 37.89 |
| ATOM | 2233 | CD1 | PHE | 329 | 23.396 | 32.760 | 17.391 | 1.00 | 37.32 |
| ATOM | 2234 | CD2 | PHE | 329 | 22.995 | 35.117 | 17.207 | 1.00 | 37.37 |
| ATOM | 2235 | CE1 | PHE | 329 | 24.185 | 32.803 | 16.243 | 1.00 | 37.40 |
| ATOM | 2236 | CE2 | PHE | 329 | 23.781 | 35.169 | 16.061 | 1.00 | 37.67 |
| ATOM | 2237 | CZ | PHE | 329 | 24.378 | 34.011 | 15.576 | 1.00 | 38.05 |
| ATOM | 2238 | C | PHE | 329 | 21.691 | 34.872 | 21.415 | 1.00 | 36.35 |
| ATOM | 2239 | O | PHE | 329 | 21.294 | 36.040 | 21.387 | 1.00 | 35.47 |
| ATOM | 2240 | N | ALA | 330 | 21.282 | 33.997 | 22.328 | 1.00 | 36.19 |
| ATOM | 2241 | CA | ALA | 330 | 20.329 | 34.382 | 23.363 | 1.00 | 37.22 |
| ATOM | 2242 | CB | ALA | 330 | 19.929 | 33.159 | 24.187 | 1.00 | 37.12 |
| ATOM | 2243 | C | ALA | 330 | 20.930 | 35.453 | 24.275 | 1.00 | 38.43 |
| ATOM | 2244 | O | ALA | 330 | 20.284 | 36.456 | 24.587 | 1.00 | 36.62 |
| ATOM | 2245 | N | LYS | 331 | 22.169 | 35.224 | 24.703 | 1.00 | 40.16 |
| ATOM | 2246 | CA | LYS | 331 | 22.877 | 36.150 | 25.583 | 1.00 | 42.64 |
| ATOM | 2247 | CB | LYS | 331 | 24.239 | 35.560 | 25.970 | 1.00 | 43.34 |
| ATOM | 2248 | CG | LYS | 331 | 25.056 | 36.401 | 26.947 | 1.00 | 45.59 |
| ATOM | 2249 | CD | LYS | 331 | 24.494 | 36.320 | 28.362 | 1.00 | 48.66 |
| ATOM | 2250 | CE | LYS | 331 | 25.388 | 37.032 | 29.378 | 1.00 | 48.88 |
| ATOM | 2251 | NZ | LYS | 331 | 25.439 | 38.510 | 29.177 | 1.00 | 50.22 |
| ATOM | 2252 | C | LYS | 331 | 23.084 | 37.497 | 24.892 | 1.00 | 42.88 |
| ATOM | 2253 | O | LYS | 331 | 23.000 | 38.554 | 25.520 | 1.00 | 44.09 |
| ATOM | 2254 | N | LEU | 332 | 23.351 | 37.446 | 23.594 | 1.00 | 43.08 |
| ATOM | 2255 | CA | LEU | 332 | 23.588 | 38.645 | 22.807 | 1.00 | 42.72 |
| ATOM | 2256 | CB | LEU | 332 | 24.020 | 38.249 | 21.393 | 1.00 | 42.60 |
| ATOM | 2257 | CG | LEU | 332 | 24.502 | 39.350 | 20.447 | 1.00 | 42.52 |
| ATOM | 2258 | CD1 | LEU | 332 | 25.490 | 38.763 | 19.448 | 1.00 | 41.43 |
| ATOM | 2259 | CD2 | LEU | 332 | 23.315 | 39.981 | 19.740 | 1.00 | 42.66 |
| ATOM | 2260 | C | LEU | 332 | 22.372 | 39.565 | 22.754 | 1.00 | 43.40 |
| ATOM | 2261 | O | LEU | 332 | 22.488 | 40.770 | 22.980 | 1.00 | 42.27 |
| ATOM | 2262 | N | PHE | 333 | 21.204 | 39.004 | 22.463 | 1.00 | 42.78 |
| ATOM | 2263 | CA | PHE | 333 | 19.997 | 39.814 | 22.387 | 1.00 | 43.66 |
| ATOM | 2264 | CB | PHE | 333 | 18.818 | 38.983 | 21.880 | 1.00 | 43.29 |
| ATOM | 2265 | CG | PHE | 333 | 19.080 | 38.289 | 20.580 | 1.00 | 43.11 |
| ATOM | 2266 | CD1 | PHE | 333 | 19.750 | 38.942 | 19.548 | 1.00 | 42.14 |
| ATOM | 2267 | CD2 | PHE | 333 | 18.640 | 36.986 | 20.379 | 1.00 | 42.92 |
| ATOM | 2268 | CE1 | PHE | 333 | 19.977 | 38.306 | 18.334 | 1.00 | 42.67 |
| ATOM | 2269 | CE2 | PHE | 333 | 18.860 | 36.341 | 19.169 | 1.00 | 42.88 |
| ATOM | 2270 | CZ | PHE | 333 | 19.531 | 37.001 | 18.141 | 1.00 | 42.87 |
| ATOM | 2271 | C | PHE | 333 | 19.651 | 40.393 | 23.747 | 1.00 | 44.42 |
| ATOM | 2272 | O | PHE | 333 | 19.189 | 41.530 | 23.851 | 1.00 | 43.85 |
| ATOM | 2273 | N | ALA | 334 | 19.881 | 39.600 | 24.789 | 1.00 | 45.14 |
| ATOM | 2274 | CA | ALA | 334 | 19.592 | 40.017 | 26.154 | 1.00 | 45.59 |
| ATOM | 2275 | CB | ALA | 334 | 19.912 | 38.884 | 27.121 | 1.00 | 45.78 |
| ATOM | 2276 | C | ALA | 334 | 20.376 | 41.263 | 26.538 | 1.00 | 45.65 |
| ATOM | 2277 | O | ALA | 334 | 19.837 | 42.179 | 27.162 | 1.00 | 46.18 |
| ATOM | 2278 | N | ASP | 335 | 21.649 | 41.296 | 26.160 | 1.00 | 45.92 |
| ATOM | 2279 | CA | ASP | 335 | 22.509 | 42.429 | 26.482 | 1.00 | 45.71 |
| ATOM | 2280 | CB | ASP | 335 | 23.977 | 42.019 | 26.388 | 1.00 | 44.57 |
| ATOM | 2281 | CG | ASP | 335 | 24.277 | 40.767 | 27.177 | 1.00 | 43.56 |
| ATOM | 2282 | OD1 | ASP | 335 | 23.536 | 40.484 | 28.141 | 1.00 | 43.70 |
| ATOM | 2283 | OD2 | ASP | 335 | 25.258 | 40.071 | 26.840 | 1.00 | 43.87 |
| ATOM | 2284 | C | ASP | 335 | 22.262 | 43.629 | 25.585 | 1.00 | 46.23 |
| ATOM | 2285 | O | ASP | 335 | 22.912 | 44.660 | 25.733 | 1.00 | 46.45 |
| ATOM | 2286 | N | GLU | 336 | 21.324 | 43.491 | 24.654 | 1.00 | 47.36 |
| ATOM | 2287 | CA | GLU | 336 | 20.999 | 44.580 | 23.741 | 1.00 | 49.00 |
| ATOM | 2288 | CB | GLU | 336 | 21.063 | 44.099 | 22.290 | 1.00 | 49.86 |
| ATOM | 2289 | CG | GLU | 336 | 22.459 | 43.725 | 21.828 | 1.00 | 51.33 |
| ATOM | 2290 | CD | GLU | 336 | 23.450 | 44.861 | 22.006 | 1.00 | 52.76 |
| ATOM | 2291 | OE1 | GLU | 336 | 23.232 | 45.943 | 21.418 | 1.00 | 53.76 |
| ATOM | 2292 | OE2 | GLU | 336 | 24.443 | 44.672 | 22.741 | 1.00 | 53.33 |
| ATOM | 2293 | C | GLU | 336 | 19.620 | 45.156 | 24.026 | 1.00 | 49.27 |
| ATOM | 2294 | O | GLU | 336 | 19.171 | 46.076 | 23.341 | 1.00 | 49.79 |
| ATOM | 2295 | N | GLY | 337 | 18.948 | 44.608 | 25.033 | 1.00 | 49.15 |
| ATOM | 2296 | CA | GLY | 337 | 17.627 | 45.094 | 25.386 | 1.00 | 49.61 |
| ATOM | 2297 | C | GLY | 337 | 16.497 | 44.169 | 24.981 | 1.00 | 50.11 |
| ATOM | 2298 | O | GLY | 337 | 15.372 | 44.318 | 25.458 | 1.00 | 49.98 |
| ATOM | 2299 | N | LEU | 338 | 16.788 | 43.219 | 24.095 | 1.00 | 50.32 |
| ATOM | 2300 | CA | LEU | 338 | 15.779 | 42.267 | 23.637 | 1.00 | 50.74 |
| ATOM | 2301 | CB | LEU | 338 | 16.108 | 41.781 | 22.223 | 1.00 | 50.90 |
| ATOM | 2302 | CG | LEU | 338 | 15.750 | 42.728 | 21.079 | 1.00 | 51.60 |
| ATOM | 2303 | CD1 | LEU | 338 | 16.237 | 42.143 | 19.763 | 1.00 | 52.47 |
| ATOM | 2304 | CD2 | LEU | 338 | 14.243 | 42.943 | 21.044 | 1.00 | 52.43 |
| ATOM | 2305 | C | LEU | 338 | 15.674 | 41.073 | 24.575 | 1.00 | 50.23 |
| ATOM | 2306 | O | LEU | 338 | 16.430 | 40.109 | 24.459 | 1.00 | 50.85 |
| ATOM | 2307 | N | ASN | 339 | 14.729 | 41.146 | 25.506 | 1.00 | 49.73 |
| ATOM | 2308 | CA | ASN | 339 | 14.514 | 40.076 | 26.469 | 1.00 | 48.47 |
| ATOM | 2309 | CB | ASN | 339 | 14.536 | 40.647 | 27.889 | 1.00 | 50.75 |
| ATOM | 2310 | CG | ASN | 339 | 15.942 | 40.986 | 28.359 | 1.00 | 52.34 |
| ATOM | 2311 | OD1 | ASN | 339 | 16.706 | 40.105 | 28.759 | 1.00 | 52.97 |
| ATOM | 2312 | ND2 | ASN | 339 | 16.293 | 42.267 | 28.301 | 1.00 | 52.70 |
| ATOM | 2313 | C | ASN | 339 | 13.194 | 39.355 | 26.204 | 1.00 | 46.90 |
| ATOM | 2314 | O | ASN | 339 | 12.490 | 38.961 | 27.129 | 1.00 | 46.89 |
| ATOM | 2315 | N | VAL | 340 | 12.865 | 39.195 | 24.927 | 1.00 | 44.67 |
| ATOM | 2316 | CA | VAL | 340 | 11.645 | 38.509 | 24.518 | 1.00 | 43.58 |
| ATOM | 2317 | CB | VAL | 340 | 10.563 | 39.501 | 24.072 | 1.00 | 43.60 |
| ATOM | 2318 | CG1 | VAL | 340 | 9.272 | 38.758 | 23.779 | 1.00 | 43.89 |
| ATOM | 2319 | CG2 | VAL | 340 | 10.343 | 40.550 | 25.147 | 1.00 | 43.92 |
| ATOM | 2320 | C | VAL | 340 | 11.991 | 37.597 | 23.345 | 1.00 | 42.70 |
| ATOM | 2321 | O | VAL | 340 | 11.806 | 37.959 | 22.182 | 1.00 | 42.88 |
| ATOM | 2322 | N | MET | 341 | 12.496 | 36.410 | 23.667 | 1.00 | 41.15 |
| ATOM | 2323 | CA | MET | 341 | 12.910 | 35.436 | 22.664 | 1.00 | 37.91 |
| ATOM | 2324 | CB | MET | 341 | 14.278 | 34.874 | 23.056 | 1.00 | 38.70 |
| ATOM | 2325 | CG | MET | 341 | 15.403 | 35.893 | 22.946 | 1.00 | 38.98 |
| ATOM | 2326 | SD | MET | 341 | 16.816 | 35.520 | 23.975 | 1.00 | 43.33 |
| ATOM | 2327 | CE | MET | 341 | 16.743 | 36.882 | 25.141 | 1.00 | 41.65 |
| ATOM | 2328 | C | MET | 341 | 11.910 | 34.299 | 22.461 | 1.00 | 36.58 |
| ATOM | 2329 | O | MET | 341 | 11.208 | 33.895 | 23.389 | 1.00 | 34.24 |
| ATOM | 2330 | N | LEU | 342 | 11.858 | 33.784 | 21.236 | 1.00 | 33.02 |
| ATOM | 2331 | CA | LEU | 342 | 10.949 | 32.697 | 20.904 | 1.00 | 31.78 |
| ATOM | 2332 | CB | LEU | 342 | 9.873 | 33.204 | 19.930 | 1.00 | 33.59 |
| ATOM | 2333 | CG | LEU | 342 | 8.868 | 32.254 | 19.260 | 1.00 | 35.82 |
| ATOM | 2334 | CD1 | LEU | 342 | 9.491 | 31.606 | 18.041 | 1.00 | 36.45 |
| ATOM | 2335 | CD2 | LEU | 342 | 8.380 | 31.210 | 20.256 | 1.00 | 35.85 |
| ATOM | 2336 | C | LEU | 342 | 11.671 | 31.485 | 20.317 | 1.00 | 28.89 |
| ATOM | 2337 | O | LEU | 342 | 12.390 | 31.594 | 19.324 | 1.00 | 28.45 |
| ATOM | 2338 | N | VAL | 343 | 11.494 | 30.334 | 20.957 | 1.00 | 25.63 |
| ATOM | 2339 | CA | VAL | 343 | 12.099 | 29.093 | 20.483 | 1.00 | 24.23 |
| ATOM | 2340 | CB | VAL | 343 | 12.543 | 28.193 | 21.664 | 1.00 | 24.85 |
| ATOM | 2341 | CG1 | VAL | 343 | 13.222 | 26.942 | 21.135 | 1.00 | 24.17 |
| ATOM | 2342 | CG2 | VAL | 343 | 13.490 | 28.960 | 22.585 | 1.00 | 24.85 |
| ATOM | 2343 | C | VAL | 343 | 11.001 | 28.393 | 19.685 | 1.00 | 23.87 |
| ATOM | 2344 | O | VAL | 343 | 10.253 | 27.575 | 20.220 | 1.00 | 23.67 |
| ATOM | 2345 | N | GLY | 344 | 10.900 | 28.737 | 18.404 | 1.00 | 23.68 |
| ATOM | 2346 | CA | GLY | 344 | 9.871 | 28.152 | 17.562 | 1.00 | 24.64 |
| ATOM | 2347 | C | GLY | 344 | 10.312 | 26.961 | 16.736 | 1.00 | 23.93 |
| ATOM | 2348 | O | GLY | 344 | 11.507 | 26.689 | 16.621 | 1.00 | 22.71 |
| ATOM | 2349 | N | ASP | 345 | 9.340 | 26.253 | 16.161 | 1.00 | 23.97 |
| ATOM | 2350 | CA | ASP | 345 | 9.625 | 25.087 | 15.342 | 1.00 | 24.30 |
| ATOM | 2351 | CB | ASP | 345 | 8.342 | 24.306 | 15.022 | 1.00 | 26.36 |
| ATOM | 2352 | CG | ASP | 345 | 7.201 | 25.196 | 14.563 | 1.00 | 26.89 |
| ATOM | 2353 | OD1 | ASP | 345 | 7.454 | 26.315 | 14.068 | 1.00 | 27.29 |
| ATOM | 2354 | OD2 | ASP | 345 | 6.042 | 24.759 | 14.691 | 1.00 | 28.98 |
| ATOM | 2355 | C | ASP | 345 | 10.337 | 25.464 | 14.051 | 1.00 | 25.12 |
| ATOM | 2356 | O | ASP | 345 | 10.707 | 24.597 | 13.260 | 1.00 | 25.35 |
| ATOM | 2357 | N | SER | 346 | 10.538 | 26.761 | 13.840 | 1.00 | 24.45 |
| ATOM | 2358 | CA | SER | 346 | 11.237 | 27.218 | 12.649 | 1.00 | 23.43 |
| ATOM | 2359 | CB | SER | 346 | 11.273 | 28.749 | 12.603 | 1.00 | 23.92 |
| ATOM | 2360 | OG | SER | 346 | 11.844 | 29.276 | 13.786 | 1.00 | 27.17 |
| ATOM | 2361 | C | SER | 346 | 12.655 | 26.655 | 12.705 | 1.00 | 23.97 |
| ATOM | 2362 | O | SER | 346 | 13.330 | 26.533 | 11.685 | 1.00 | 24.80 |
| ATOM | 2363 | N | LEU | 347 | 13.090 | 26.295 | 13.909 | 1.00 | 22.27 |
| ATOM | 2364 | CA | LEU | 347 | 14.417 | 25.730 | 14.106 | 1.00 | 22.38 |
| ATOM | 2365 | CB | LEU | 347 | 14.664 | 25.473 | 15.601 | 1.00 | 22.34 |
| ATOM | 2366 | CG | LEU | 347 | 13.793 | 24.461 | 16.355 | 1.00 | 23.80 |
| ATOM | 2367 | CD1 | LEU | 347 | 14.374 | 23.052 | 16.197 | 1.00 | 21.37 |
| ATOM | 2368 | CD2 | LEU | 347 | 13.746 | 24.838 | 17.831 | 1.00 | 22.86 |
| ATOM | 2369 | C | LEU | 347 | 14.548 | 24.426 | 13.313 | 1.00 | 21.48 |
| ATOM | 2370 | O | LEU | 347 | 15.653 | 23.970 | 13.033 | 1.00 | 22.19 |
| ATOM | 2371 | N | GLY | 348 | 13.413 | 23.835 | 12.955 | 1.00 | 22.76 |
| ATOM | 2372 | CA | GLY | 348 | 13.439 | 22.601 | 12.196 | 1.00 | 23.34 |
| ATOM | 2373 | C | GLY | 348 | 14.005 | 22.837 | 10.814 | 1.00 | 24.59 |
| ATOM | 2374 | O | GLY | 348 | 14.534 | 21.927 | 10.179 | 1.00 | 24.89 |
| ATOM | 2375 | N | MET | 349 | 13.908 | 24.078 | 10.351 | 1.00 | 25.57 |
| ATOM | 2376 | CA | MET | 349 | 14.408 | 24.431 | 9.034 | 1.00 | 27.75 |
| ATOM | 2377 | CB | MET | 349 | 13.349 | 25.255 | 8.290 | 1.00 | 28.73 |
| ATOM | 2378 | CG | MET | 349 | 12.062 | 24.479 | 8.029 | 1.00 | 32.37 |
| ATOM | 2379 | SD | MET | 349 | 10.740 | 25.431 | 7.229 | 1.00 | 34.95 |
| ATOM | 2380 | CE | MET | 349 | 11.415 | 25.598 | 5.580 | 1.00 | 34.51 |
| ATOM | 2381 | C | MET | 349 | 15.729 | 25.191 | 9.118 | 1.00 | 27.11 |
| ATOM | 2382 | O | MET | 349 | 16.700 | 24.839 | 8.448 | 1.00 | 26.59 |
| ATOM | 2383 | N | THR | 350 | 15.774 | 26.205 | 9.975 | 1.00 | 27.84 |
| ATOM | 2384 | CA | THR | 350 | 16.965 | 27.031 | 10.133 | 1.00 | 28.17 |
| ATOM | 2385 | CB | THR | 350 | 16.594 | 28.375 | 10.805 | 1.00 | 31.28 |
| ATOM | 2386 | OG1 | THR | 350 | 17.720 | 29.258 | 10.770 | 1.00 | 34.87 |
| ATOM | 2387 | CG2 | THR | 350 | 16.181 | 28.151 | 12.249 | 1.00 | 31.41 |
| ATOM | 2388 | C | THR | 350 | 18.098 | 26.370 | 10.926 | 1.00 | 28.30 |
| ATOM | 2389 | O | THR | 350 | 19.275 | 26.666 | 10.713 | 1.00 | 27.38 |
| ATOM | 2390 | N | VAL | 351 | 17.747 | 25.470 | 11.836 | 1.00 | 26.54 |
| ATOM | 2391 | CA | VAL | 351 | 18.750 | 24.792 | 12.648 | 1.00 | 26.15 |
| ATOM | 2392 | CB | VAL | 351 | 18.337 | 24.800 | 14.136 | 1.00 | 27.12 |
| ATOM | 2393 | CG1 | VAL | 351 | 19.340 | 24.010 | 14.966 | 1.00 | 26.33 |
| ATOM | 2394 | CG2 | VAL | 351 | 18.238 | 26.239 | 14.633 | 1.00 | 26.84 |
| ATOM | 2395 | C | VAL | 351 | 18.995 | 23.347 | 12.214 | 1.00 | 25.31 |
| ATOM | 2396 | O | VAL | 351 | 20.138 | 22.943 | 12.023 | 1.00 | 23.95 |
| ATOM | 2397 | N | GLN | 352 | 17.920 | 22.574 | 12.064 | 1.00 | 24.36 |
| ATOM | 2398 | CA | GLN | 352 | 18.036 | 21.169 | 11.673 | 1.00 | 23.55 |
| ATOM | 2399 | CB | GLN | 352 | 16.839 | 20.370 | 12.200 | 1.00 | 23.18 |
| ATOM | 2400 | CG | GLN | 352 | 16.508 | 20.642 | 13.670 | 1.00 | 21.36 |
| ATOM | 2401 | CD | GLN | 352 | 15.365 | 19.785 | 14.186 | 1.00 | 20.35 |
| ATOM | 2402 | OE1 | GLN | 352 | 14.468 | 19.417 | 13.434 | 1.00 | 19.01 |
| ATOM | 2403 | NE2 | GLN | 352 | 15.387 | 19.476 | 15.484 | 1.00 | 18.25 |
| ATOM | 2404 | C | GLN | 352 | 18.156 | 20.972 | 10.159 | 1.00 | 24.30 |
| ATOM | 2405 | O | GLN | 352 | 18.727 | 19.982 | 9.703 | 1.00 | 24.86 |
| ATOM | 2406 | N | GLY | 353 | 17.602 | 21.900 | 9.385 | 1.00 | 24.94 |
| ATOM | 2407 | CA | GLY | 353 | 17.688 | 21.796 | 7.938 | 1.00 | 26.08 |
| ATOM | 2408 | C | GLY | 353 | 16.607 | 20.985 | 7.243 | 1.00 | 28.43 |
| ATOM | 2409 | O | GLY | 353 | 16.866 | 20.335 | 6.229 | 1.00 | 29.40 |
| ATOM | 2410 | N | HIS | 354 | 15.393 | 21.020 | 7.781 | 1.00 | 28.07 |
| ATOM | 2411 | CA | HIS | 354 | 14.280 | 20.291 | 7.192 | 1.00 | 29.87 |
| ATOM | 2412 | CB | HIS | 354 | 13.328 | 19.807 | 8.286 | 1.00 | 28.92 |
| ATOM | 2413 | CG | HIS | 354 | 13.906 | 18.740 | 9.161 | 1.00 | 28.40 |
| ATOM | 2414 | CD2 | HIS | 354 | 14.175 | 18.722 | 10.488 | 1.00 | 28.09 |
| ATOM | 2415 | ND1 | HIS | 354 | 14.272 | 17.501 | 8.679 | 1.00 | 28.16 |
| ATOM | 2416 | CE1 | HIS | 354 | 14.741 | 16.766 | 9.671 | 1.00 | 28.40 |
| ATOM | 2417 | NE2 | HIS | 354 | 14.693 | 17.484 | 10.780 | 1.00 | 27.51 |
| ATOM | 2418 | C | HIS | 354 | 13.520 | 21.162 | 6.198 | 1.00 | 31.29 |
| ATOM | 2419 | O | HIS | 354 | 13.625 | 22.389 | 6.227 | 1.00 | 31.35 |
| ATOM | 2420 | N | ASP | 355 | 12.755 | 20.518 | 5.320 | 1.00 | 33.89 |
| ATOM | 2421 | CA | ASP | 355 | 11.972 | 21.224 | 4.305 | 1.00 | 34.61 |
| ATOM | 2422 | CB | ASP | 355 | 11.479 | 20.231 | 3.245 | 1.00 | 38.73 |
| ATOM | 2423 | CG | ASP | 355 | 10.673 | 19.085 | 3.842 | 1.00 | 41.27 |
| ATOM | 2424 | OD1 | ASP | 355 | 9.606 | 19.349 | 4.430 | 1.00 | 43.21 |
| ATOM | 2425 | OD2 | ASP | 355 | 11.109 | 17.916 | 3.719 | 1.00 | 44.12 |
| ATOM | 2426 | C | ASP | 355 | 10.786 | 21.953 | 4.928 | 1.00 | 34.04 |
| ATOM | 2427 | O | ASP | 355 | 10.248 | 22.898 | 4.348 | 1.00 | 33.77 |
| ATOM | 2428 | N | SER | 356 | 10.382 | 21.503 | 6.111 | 1.00 | 31.25 |
| ATOM | 2429 | CA | SER | 356 | 9.268 | 22.106 | 6.832 | 1.00 | 29.42 |
| ATOM | 2430 | CB | SER | 356 | 7.963 | 21.353 | 6.531 | 1.00 | 29.55 |
| ATOM | 2431 | OG | SER | 356 | 7.976 | 20.046 | 7.086 | 1.00 | 30.40 |
| ATOM | 2432 | C | SER | 356 | 9.564 | 22.058 | 8.330 | 1.00 | 26.94 |
| ATOM | 2433 | O | SER | 356 | 10.642 | 21.627 | 8.739 | 1.00 | 26.83 |
| ATOM | 2434 | N | THR | 357 | 8.612 | 22.498 | 9.145 | 1.00 | 26.28 |
| ATOM | 2435 | CA | THR | 357 | 8.803 | 22.491 | 10.592 | 1.00 | 24.80 |
| ATOM | 2436 | CB | THR | 357 | 8.205 | 23.749 | 11.254 | 1.00 | 23.50 |
| ATOM | 2437 | OG1 | THR | 357 | 6.780 | 23.735 | 11.093 | 1.00 | 25.56 |
| ATOM | 2438 | CG2 | THR | 357 | 8.777 | 25.017 | 10.630 | 1.00 | 26.01 |
| ATOM | 2439 | C | THR | 357 | 8.141 | 21.288 | 11.252 | 1.00 | 22.76 |
| ATOM | 2440 | O | THR | 357 | 8.262 | 21.106 | 12.458 | 1.00 | 21.76 |
| ATOM | 2441 | N | LEU | 358 | 7.449 | 20.466 | 10.470 | 1.00 | 21.94 |
| ATOM | 2442 | CA | LEU | 358 | 6.757 | 19.306 | 11.036 | 1.00 | 20.73 |
| ATOM | 2443 | CB | LEU | 358 | 5.987 | 18.553 | 9.946 | 1.00 | 22.06 |
| ATOM | 2444 | CG | LEU | 358 | 4.696 | 19.207 | 9.447 | 1.00 | 22.23 |
| ATOM | 2445 | CD1 | LEU | 358 | 5.036 | 20.431 | 8.609 | 1.00 | 24.54 |
| ATOM | 2446 | CD2 | LEU | 358 | 3.901 | 18.213 | 8.623 | 1.00 | 21.61 |
| ATOM | 2447 | C | LEU | 358 | 7.620 | 18.311 | 11.807 | 1.00 | 19.69 |
| ATOM | 2448 | O | LEU | 358 | 7.169 | 17.732 | 12.792 | 1.00 | 19.43 |
| ATOM | 2449 | N | PRO | 359 | 8.861 | 18.077 | 11.359 | 1.00 | 19.49 |
| ATOM | 2450 | CD | PRO | 359 | 9.445 | 18.471 | 10.067 | 1.00 | 20.99 |
| ATOM | 2451 | CA | PRO | 359 | 9.738 | 17.130 | 12.057 | 1.00 | 19.78 |
| ATOM | 2452 | CB | PRO | 359 | 10.917 | 16.974 | 11.095 | 1.00 | 21.23 |
| ATOM | 2453 | CG | PRO | 359 | 10.905 | 18.271 | 10.325 | 1.00 | 25.57 |
| ATOM | 2454 | C | PRO | 359 | 10.184 | 17.534 | 13.461 | 1.00 | 18.27 |
| ATOM | 2455 | O | PRO | 359 | 10.685 | 16.705 | 14.225 | 1.00 | 16.56 |
| ATOM | 2456 | N | VAL | 360 | 9.993 | 18.800 | 13.806 | 1.00 | 17.80 |
| ATOM | 2457 | CA | VAL | 360 | 10.397 | 19.290 | 15.116 | 1.00 | 17.74 |
| ATOM | 2458 | CB | VAL | 360 | 10.204 | 20.816 | 15.226 | 1.00 | 16.25 |
| ATOM | 2459 | CG1 | VAL | 360 | 10.676 | 21.301 | 16.582 | 1.00 | 15.89 |
| ATOM | 2460 | CG2 | VAL | 360 | 10.967 | 21.508 | 14.114 | 1.00 | 16.99 |
| ATOM | 2461 | C | VAL | 360 | 9.589 | 18.608 | 16.205 | 1.00 | 17.81 |
| ATOM | 2462 | O | VAL | 360 | 8.362 | 18.577 | 16.152 | 1.00 | 16.42 |
| ATOM | 2463 | N | THR | 361 | 10.280 | 18.056 | 17.195 | 1.00 | 18.83 |
| ATOM | 2464 | CA | THR | 361 | 9.594 | 17.376 | 18.283 | 1.00 | 19.94 |
| ATOM | 2465 | CB | THR | 361 | 10.216 | 15.983 | 18.540 | 1.00 | 23.20 |
| ATOM | 2466 | OG1 | THR | 361 | 10.537 | 15.352 | 17.289 | 1.00 | 26.54 |
| ATOM | 2467 | CG2 | THR | 361 | 9.221 | 15.091 | 19.257 | 1.00 | 27.25 |
| ATOM | 2468 | C | THR | 361 | 9.656 | 18.195 | 19.571 | 1.00 | 19.28 |
| ATOM | 2469 | O | THR | 361 | 10.442 | 19.138 | 19.679 | 1.00 | 17.95 |
| ATOM | 2470 | N | VAL | 362 | 8.820 | 17.841 | 20.544 | 1.00 | 17.01 |
| ATOM | 2471 | CA | VAL | 362 | 8.807 | 18.545 | 21.827 | 1.00 | 17.54 |
| ATOM | 2472 | CB | VAL | 362 | 7.753 | 17.930 | 22.787 | 1.00 | 16.12 |
| ATOM | 2473 | CG1 | VAL | 362 | 7.835 | 18.598 | 24.156 | 1.00 | 15.29 |
| ATOM | 2474 | CG2 | VAL | 362 | 6.352 | 18.108 | 22.198 | 1.00 | 16.25 |
| ATOM | 2475 | C | VAL | 362 | 10.202 | 18.463 | 22.450 | 1.00 | 16.96 |
| ATOM | 2476 | O | VAL | 362 | 10.703 | 19.445 | 22.989 | 1.00 | 16.86 |
| ATOM | 2477 | N | ALA | 363 | 10.829 | 17.293 | 22.351 | 1.00 | 17.98 |
| ATOM | 2478 | CA | ALA | 363 | 12.164 | 17.088 | 22.903 | 1.00 | 18.14 |
| ATOM | 2479 | CB | ALA | 363 | 12.638 | 15.659 | 22.632 | 1.00 | 20.14 |
| ATOM | 2480 | C | ALA | 363 | 13.161 | 18.086 | 22.327 | 1.00 | 18.21 |
| ATOM | 2481 | O | ALA | 363 | 14.042 | 18.574 | 23.043 | 1.00 | 17.54 |
| ATOM | 2482 | N | ASP | 364 | 13.029 | 18.378 | 21.032 | 1.00 | 16.91 |
| ATOM | 2483 | CA | ASP | 364 | 13.923 | 19.334 | 20.371 | 1.00 | 16.57 |
| ATOM | 2484 | CB | ASP | 364 | 13.661 | 19.410 | 18.854 | 1.00 | 15.99 |
| ATOM | 2485 | CG | ASP | 364 | 13.951 | 18.099 | 18.123 | 1.00 | 17.52 |
| ATOM | 2486 | OD1 | ASP | 364 | 14.817 | 17.312 | 18.572 | 1.00 | 15.98 |
| ATOM | 2487 | OD2 | ASP | 364 | 13.311 | 17.873 | 17.072 | 1.00 | 19.08 |
| ATOM | 2488 | C | ASP | 364 | 13.691 | 20.720 | 20.974 | 1.00 | 15.38 |
| ATOM | 2489 | O | ASP | 364 | 14.638 | 21.421 | 21.320 | 1.00 | 17.32 |
| ATOM | 2490 | N | ILE | 365 | 12.427 | 21.115 | 21.088 | 1.00 | 17.64 |
| ATOM | 2491 | CA | ILE | 365 | 12.092 | 22.422 | 21.651 | 1.00 | 16.80 |
| ATOM | 2492 | CB | ILE | 365 | 10.561 | 22.646 | 21.717 | 1.00 | 16.19 |
| ATOM | 2493 | CG2 | ILE | 365 | 10.265 | 23.986 | 22.395 | 1.00 | 19.39 |
| ATOM | 2494 | CG1 | ILE | 365 | 9.946 | 22.599 | 20.310 | 1.00 | 16.26 |
| ATOM | 2495 | CD1 | ILE | 365 | 10.399 | 23.713 | 19.379 | 1.00 | 17.28 |
| ATOM | 2496 | C | ILE | 365 | 12.646 | 22.560 | 23.066 | 1.00 | 16.75 |
| ATOM | 2497 | O | ILE | 365 | 13.217 | 23.596 | 23.423 | 1.00 | 15.83 |
| ATOM | 2498 | N | ALA | 366 | 12.474 | 21.518 | 23.878 | 1.00 | 15.24 |
| ATOM | 2499 | CA | ALA | 366 | 12.959 | 21.549 | 25.260 | 1.00 | 15.66 |
| ATOM | 2500 | CB | ALA | 366 | 12.533 | 20.278 | 25.999 | 1.00 | 15.05 |
| ATOM | 2501 | C | ALA | 366 | 14.474 | 21.688 | 25.292 | 1.00 | 15.90 |
| ATOM | 2502 | O | ALA | 366 | 15.036 | 22.363 | 26.161 | 1.00 | 16.01 |
| ATOM | 2503 | N | TYR | 367 | 15.136 | 21.040 | 24.341 | 1.00 | 15.21 |
| ATOM | 2504 | CA | TYR | 367 | 16.596 | 21.093 | 24.247 | 1.00 | 15.10 |
| ATOM | 2505 | CB | TYR | 367 | 17.082 | 20.191 | 23.106 | 1.00 | 14.11 |
| ATOM | 2506 | CG | TYR | 367 | 18.577 | 20.270 | 22.837 | 1.00 | 15.47 |
| ATOM | 2507 | CD1 | TYR | 367 | 19.504 | 19.782 | 23.755 | 1.00 | 15.21 |
| ATOM | 2508 | CE1 | TYR | 367 | 20.881 | 19.830 | 23.492 | 1.00 | 16.91 |
| ATOM | 2509 | CD2 | TYR | 367 | 19.060 | 20.815 | 21.651 | 1.00 | 17.33 |
| ATOM | 2510 | CE2 | TYR | 367 | 20.428 | 20.868 | 21.382 | 1.00 | 17.98 |
| ATOM | 2511 | CZ | TYR | 367 | 21.330 | 20.371 | 22.306 | 1.00 | 16.29 |
| ATOM | 2512 | OH | TYR | 367 | 22.681 | 20.386 | 22.013 | 1.00 | 15.79 |
| ATOM | 2513 | C | TYR | 367 | 17.051 | 22.525 | 23.986 | 1.00 | 16.84 |
| ATOM | 2514 | O | TYR | 367 | 17.918 | 23.063 | 24.688 | 1.00 | 17.12 |
| ATOM | 2515 | N | HIS | 368 | 16.467 | 23.141 | 22.965 | 1.00 | 17.61 |
| ATOM | 2516 | CA | HIS | 368 | 16.831 | 24.511 | 22.611 | 1.00 | 19.23 |
| ATOM | 2517 | CB | HIS | 368 | 16.277 | 24.847 | 21.220 | 1.00 | 18.64 |
| ATOM | 2518 | CG | HIS | 368 | 16.970 | 24.112 | 20.114 | 1.00 | 19.41 |
| ATOM | 2519 | CD2 | HIS | 368 | 16.608 | 23.014 | 19.409 | 1.00 | 19.84 |
| ATOM | 2520 | ND1 | HIS | 368 | 18.241 | 24.438 | 19.690 | 1.00 | 20.14 |
| ATOM | 2521 | CE1 | HIS | 368 | 18.633 | 23.570 | 18.775 | 1.00 | 20.20 |
| ATOM | 2522 | NE2 | HIS | 368 | 17.662 | 22.694 | 18.587 | 1.00 | 20.30 |
| ATOM | 2523 | C | HIS | 368 | 16.360 | 25.516 | 23.661 | 1.00 | 20.01 |
| ATOM | 2524 | O | HIS | 368 | 17.047 | 26.500 | 23.936 | 1.00 | 21.66 |
| ATOM | 2525 | N | THR | 369 | 15.202 | 25.258 | 24.259 | 1.00 | 19.85 |
| ATOM | 2526 | CA | THR | 369 | 14.677 | 26.143 | 25.289 | 1.00 | 21.15 |
| ATOM | 2527 | CB | THR | 369 | 13.305 | 25.659 | 25.796 | 1.00 | 21.21 |
| ATOM | 2528 | OG1 | THR | 369 | 12.336 | 25.804 | 24.750 | 1.00 | 22.15 |
| ATOM | 2529 | CG2 | THR | 369 | 12.860 | 26.466 | 27.012 | 1.00 | 21.16 |
| ATOM | 2530 | C | THR | 369 | 15.634 | 26.241 | 26.474 | 1.00 | 22.56 |
| ATOM | 2531 | O | THR | 369 | 15.905 | 27.338 | 26.974 | 1.00 | 23.65 |
| ATOM | 2532 | N | ALA | 370 | 16.154 | 25.100 | 26.918 | 1.00 | 21.27 |
| ATOM | 2533 | CA | ALA | 370 | 17.078 | 25.078 | 28.051 | 1.00 | 23.22 |
| ATOM | 2534 | CB | ALA | 370 | 17.481 | 23.640 | 28.372 | 1.00 | 22.74 |
| ATOM | 2535 | C | ALA | 370 | 18.322 | 25.913 | 27.752 | 1.00 | 24.33 |
| ATOM | 2536 | O | ALA | 370 | 18.775 | 26.694 | 28.593 | 1.00 | 24.36 |
| ATOM | 2537 | N | ALA | 371 | 18.862 | 25.744 | 26.548 | 1.00 | 24.54 |
| ATOM | 2538 | CA | ALA | 371 | 20.050 | 26.480 | 26.115 | 1.00 | 26.15 |
| ATOM | 2539 | CB | ALA | 371 | 20.465 | 26.019 | 24.719 | 1.00 | 24.54 |
| ATOM | 2540 | C | ALA | 371 | 19.795 | 27.986 | 26.109 | 1.00 | 27.22 |
| ATOM | 2541 | O | ALA | 371 | 20.610 | 28.766 | 26.601 | 1.00 | 29.91 |
| ATOM | 2542 | N | VAL | 372 | 18.662 | 28.387 | 25.543 | 1.00 | 27.69 |
| ATOM | 2543 | CA | VAL | 372 | 18.299 | 29.796 | 25.473 | 1.00 | 28.62 |
| ATOM | 2544 | CB | VAL | 372 | 16.975 | 29.992 | 24.699 | 1.00 | 29.37 |
| ATOM | 2545 | CG1 | VAL | 372 | 16.541 | 31.448 | 24.749 | 1.00 | 28.12 |
| ATOM | 2546 | CG2 | VAL | 372 | 17.154 | 29.546 | 23.257 | 1.00 | 29.50 |
| ATOM | 2547 | C | VAL | 372 | 18.153 | 30.393 | 26.868 | 1.00 | 29.28 |
| ATOM | 2548 | O | VAL | 372 | 18.633 | 31.499 | 27.130 | 1.00 | 28.13 |
| ATOM | 2549 | N | ARG | 373 | 17.496 | 29.661 | 27.762 | 1.00 | 29.17 |
| ATOM | 2550 | CA | ARG | 373 | 17.299 | 30.138 | 29.128 | 1.00 | 29.87 |
| ATOM | 2551 | CB | ARG | 373 | 16.500 | 29.118 | 29.951 | 1.00 | 29.61 |
| ATOM | 2552 | CG | ARG | 373 | 16.378 | 29.474 | 31.437 | 1.00 | 29.17 |
| ATOM | 2553 | CD | ARG | 373 | 15.773 | 30.858 | 31.623 | 1.00 | 26.49 |
| ATOM | 2554 | NE | ARG | 373 | 14.370 | 30.903 | 31.228 | 1.00 | 28.14 |
| ATOM | 2555 | CZ | ARG | 373 | 13.703 | 32.022 | 30.961 | 1.00 | 27.87 |
| ATOM | 2556 | NH1 | ARG | 373 | 14.307 | 33.199 | 31.042 | 1.00 | 27.29 |
| ATOM | 2557 | NH2 | ARG | 373 | 12.427 | 31.967 | 30.614 | 1.00 | 28.85 |
| ATOM | 2558 | C | ARG | 373 | 18.630 | 30.415 | 29.811 | 1.00 | 31.14 |
| ATOM | 2559 | O | ARG | 373 | 18.763 | 31.394 | 30.547 | 1.00 | 31.51 |
| ATOM | 2560 | N | ARG | 374 | 19.615 | 29.554 | 29.573 | 1.00 | 32.05 |
| ATOM | 2561 | CA | ARG | 374 | 20.928 | 29.743 | 30.175 | 1.00 | 34.06 |
| ATOM | 2562 | CB | ARG | 374 | 21.873 | 28.597 | 29.798 | 1.00 | 33.29 |
| ATOM | 2563 | CG | ARG | 374 | 21.388 | 27.221 | 30.202 | 1.00 | 33.93 |
| ATOM | 2564 | CD | ARG | 374 | 22.522 | 26.212 | 30.150 | 1.00 | 32.70 |
| ATOM | 2565 | NE | ARG | 374 | 22.071 | 24.854 | 30.450 | 1.00 | 33.93 |
| ATOM | 2566 | CZ | ARG | 374 | 21.510 | 24.033 | 29.565 | 1.00 | 33.76 |
| ATOM | 2567 | NH1 | ARG | 374 | 21.329 | 24.423 | 28.311 | 1.00 | 30.05 |
| ATOM | 2568 | NH2 | ARG | 374 | 21.131 | 22.817 | 29.936 | 1.00 | 33.89 |
| ATOM | 2569 | C | ARG | 374 | 21.532 | 31.066 | 29.713 | 1.00 | 34.72 |
| ATOM | 2570 | O | ARG | 374 | 22.160 | 31.780 | 30.496 | 1.00 | 36.00 |
| ATOM | 2571 | N | GLY | 375 | 21.331 | 31.388 | 28.439 | 1.00 | 35.42 |
| ATOM | 2572 | CA | GLY | 375 | 21.866 | 32.621 | 27.892 | 1.00 | 36.12 |
| ATOM | 2573 | C | GLY | 375 | 21.128 | 33.871 | 28.331 | 1.00 | 36.83 |
| ATOM | 2574 | O | GLY | 375 | 21.711 | 34.954 | 28.352 | 1.00 | 36.60 |
| ATOM | 2575 | N | ALA | 376 | 19.851 | 33.724 | 28.683 | 1.00 | 36.57 |
| ATOM | 2576 | CA | ALA | 376 | 19.027 | 34.851 | 29.114 | 1.00 | 37.33 |
| ATOM | 2577 | CB | ALA | 376 | 18.332 | 35.474 | 27.906 | 1.00 | 36.41 |
| ATOM | 2578 | C | ALA | 376 | 17.985 | 34.414 | 30.145 | 1.00 | 37.86 |
| ATOM | 2579 | O | ALA | 376 | 16.805 | 34.265 | 29.827 | 1.00 | 38.51 |
| ATOM | 2580 | N | PRO | 377 | 18.410 | 34.215 | 31.401 | 1.00 | 38.37 |
| ATOM | 2581 | CD | PRO | 377 | 19.789 | 34.359 | 31.901 | 1.00 | 38.55 |
| ATOM | 2582 | CA | PRO | 377 | 17.509 | 33.790 | 32.478 | 1.00 | 38.64 |
| ATOM | 2583 | CB | PRO | 377 | 18.474 | 33.389 | 33.586 | 1.00 | 39.17 |
| ATOM | 2584 | CG | PRO | 377 | 19.588 | 34.359 | 33.404 | 1.00 | 38.79 |
| ATOM | 2585 | C | PRO | 377 | 16.513 | 34.851 | 32.936 | 1.00 | 38.71 |
| ATOM | 2586 | O | PRO | 377 | 15.691 | 34.596 | 33.814 | 1.00 | 38.56 |
| ATOM | 2587 | N | ASN | 378 | 16.580 | 36.033 | 32.334 | 1.00 | 38.88 |
| ATOM | 2588 | CA | ASN | 378 | 15.682 | 37.120 | 32.707 | 1.00 | 39.81 |
| ATOM | 2589 | CB | ASN | 378 | 16.490 | 38.288 | 33.279 | 1.00 | 41.15 |
| ATOM | 2590 | CG | ASN | 378 | 17.316 | 37.889 | 34.484 | 1.00 | 41.16 |
| ATOM | 2591 | OD1 | ASN | 378 | 16.779 | 37.468 | 35.504 | 1.00 | 42.22 |
| ATOM | 2592 | ND2 | ASN | 378 | 18.632 | 38.018 | 34.369 | 1.00 | 43.85 |
| ATOM | 2593 | C | ASN | 378 | 14.838 | 37.613 | 31.540 | 1.00 | 39.91 |
| ATOM | 2594 | O | ASN | 378 | 14.375 | 38.754 | 31.541 | 1.00 | 42.79 |
| ATOM | 2595 | N | CYS | 379 | 14.635 | 36.763 | 30.541 | 1.00 | 38.30 |
| ATOM | 2596 | CA | CYS | 379 | 13.843 | 37.164 | 29.383 | 1.00 | 36.58 |
| ATOM | 2597 | CB | CYS | 379 | 14.585 | 36.836 | 28.088 | 1.00 | 36.64 |
| ATOM | 2598 | SG | CYS | 379 | 14.422 | 35.102 | 27.558 | 1.00 | 37.37 |
| ATOM | 2599 | C | CYS | 379 | 12.494 | 36.460 | 29.355 | 1.00 | 34.98 |
| ATOM | 2600 | O | CYS | 379 | 12.290 | 35.465 | 30.039 | 1.00 | 35.06 |
| ATOM | 2601 | N | LEU | 380 | 11.576 | 37.002 | 28.565 | 1.00 | 33.89 |
| ATOM | 2602 | CA | LEU | 380 | 10.261 | 36.411 | 28.397 | 1.00 | 32.87 |
| ATOM | 2603 | CB | LEU | 380 | 9.261 | 37.459 | 27.910 | 1.00 | 34.44 |
| ATOM | 2604 | CG | LEU | 380 | 7.810 | 36.999 | 27.743 | 1.00 | 34.49 |
| ATOM | 2605 | CD1 | LEU | 380 | 7.243 | 36.590 | 29.094 | 1.00 | 36.11 |
| ATOM | 2606 | CD2 | LEU | 380 | 6.982 | 38.120 | 27.139 | 1.00 | 36.04 |
| ATOM | 2607 | C | LEU | 380 | 10.485 | 35.353 | 27.319 | 1.00 | 32.33 |
| ATOM | 2608 | O | LEU | 380 | 10.675 | 35.686 | 26.147 | 1.00 | 31.37 |
| ATOM | 2609 | N | LEU | 381 | 10.477 | 34.083 | 27.715 | 1.00 | 30.46 |
| ATOM | 2610 | CA | LEU | 381 | 10.726 | 32.994 | 26.775 | 1.00 | 28.43 |
| ATOM | 2611 | CB | LEU | 381 | 11.683 | 31.976 | 27.416 | 1.00 | 28.20 |
| ATOM | 2612 | CG | LEU | 381 | 12.577 | 31.092 | 26.539 | 1.00 | 29.26 |
| ATOM | 2613 | CD1 | LEU | 381 | 13.529 | 30.320 | 27.435 | 1.00 | 28.20 |
| ATOM | 2614 | CD2 | LEU | 381 | 11.746 | 30.134 | 25.695 | 1.00 | 31.72 |
| ATOM | 2615 | C | LEU | 381 | 9.463 | 32.284 | 26.308 | 1.00 | 26.84 |
| ATOM | 2616 | O | LEU | 381 | 8.751 | 31.672 | 27.104 | 1.00 | 25.66 |
| ATOM | 2617 | N | LEU | 382 | 9.184 | 32.377 | 25.013 | 1.00 | 25.90 |
| ATOM | 2618 | CA | LEU | 382 | 8.026 | 31.706 | 24.436 | 1.00 | 26.12 |
| ATOM | 2619 | CB | LEU | 382 | 7.317 | 32.600 | 23.415 | 1.00 | 27.68 |
| ATOM | 2620 | CG | LEU | 382 | 6.383 | 33.685 | 23.955 | 1.00 | 29.10 |
| ATOM | 2621 | CD1 | LEU | 382 | 7.172 | 34.713 | 24.751 | 1.00 | 29.94 |
| ATOM | 2622 | CD2 | LEU | 382 | 5.667 | 34.344 | 22.779 | 1.00 | 30.46 |
| ATOM | 2623 | C | LEU | 382 | 8.528 | 30.450 | 23.743 | 1.00 | 24.87 |
| ATOM | 2624 | O | LEU | 382 | 9.485 | 30.499 | 22.973 | 1.00 | 24.72 |
| ATOM | 2625 | N | ALA | 383 | 7.888 | 29.323 | 24.027 | 1.00 | 24.02 |
| ATOM | 2626 | CA | ALA | 383 | 8.286 | 28.060 | 23.422 | 1.00 | 22.33 |
| ATOM | 2627 | CB | ALA | 383 | 8.631 | 27.050 | 24.503 | 1.00 | 22.02 |
| ATOM | 2628 | C | ALA | 383 | 7.157 | 27.530 | 22.567 | 1.00 | 20.94 |
| ATOM | 2629 | O | ALA | 383 | 6.016 | 27.446 | 23.017 | 1.00 | 20.60 |
| ATOM | 2630 | N | ASP | 384 | 7.467 | 27.165 | 21.331 | 1.00 | 20.39 |
| ATOM | 2631 | CA | ASP | 384 | 6.440 | 26.631 | 20.448 | 1.00 | 20.57 |
| ATOM | 2632 | CB | ASP | 384 | 6.888 | 26.689 | 18.995 | 1.00 | 22.20 |
| ATOM | 2633 | CG | ASP | 384 | 6.278 | 27.847 | 18.232 | 1.00 | 24.06 |
| ATOM | 2634 | OD1 | ASP | 384 | 5.235 | 28.372 | 18.661 | 1.00 | 26.53 |
| ATOM | 2635 | OD2 | ASP | 384 | 6.842 | 28.206 | 17.187 | 1.00 | 26.60 |
| ATOM | 2636 | C | ASP | 384 | 6.139 | 25.180 | 20.751 | 1.00 | 18.93 |
| ATOM | 2637 | O | ASP | 384 | 7.027 | 24.425 | 21.139 | 1.00 | 19.05 |
| ATOM | 2638 | N | LEU | 385 | 4.877 | 24.800 | 20.590 | 1.00 | 19.70 |
| ATOM | 2639 | CA | LEU | 385 | 4.504 | 23.398 | 20.725 | 1.00 | 19.65 |
| ATOM | 2640 | CB | LEU | 385 | 3.133 | 23.222 | 21.374 | 1.00 | 17.71 |
| ATOM | 2641 | CG | LEU | 385 | 3.087 | 23.370 | 22.901 | 1.00 | 16.98 |
| ATOM | 2642 | CD1 | LEU | 385 | 1.716 | 22.963 | 23.429 | 1.00 | 15.13 |
| ATOM | 2643 | CD2 | LEU | 385 | 4.162 | 22.498 | 23.523 | 1.00 | 16.02 |
| ATOM | 2644 | C | LEU | 385 | 4.462 | 23.012 | 19.246 | 1.00 | 20.97 |
| ATOM | 2645 | O | LEU | 385 | 3.705 | 23.596 | 18.461 | 1.00 | 21.82 |
| ATOM | 2646 | N | PRO | 386 | 5.303 | 22.047 | 18.841 | 1.00 | 19.88 |
| ATOM | 2647 | CD | PRO | 386 | 6.168 | 21.273 | 19.747 | 1.00 | 20.26 |
| ATOM | 2648 | CA | PRO | 386 | 5.416 | 21.554 | 17.466 | 1.00 | 19.86 |
| ATOM | 2649 | CB | PRO | 386 | 6.626 | 20.633 | 17.537 | 1.00 | 21.72 |
| ATOM | 2650 | CG | PRO | 386 | 6.512 | 20.061 | 18.921 | 1.00 | 20.75 |
| ATOM | 2651 | C | PRO | 386 | 4.184 | 20.846 | 16.905 | 1.00 | 19.67 |
| ATOM | 2652 | O | PRO | 386 | 3.167 | 20.679 | 17.586 | 1.00 | 19.34 |
| ATOM | 2653 | N | PHE | 387 | 4.300 | 20.437 | 15.647 | 1.00 | 19.25 |
| ATOM | 2654 | CA | PHE | 387 | 3.248 | 19.739 | 14.927 | 1.00 | 18.90 |
| ATOM | 2655 | CB | PHE | 387 | 3.820 | 19.249 | 13.580 | 1.00 | 20.39 |
| ATOM | 2656 | CG | PHE | 387 | 2.955 | 18.253 | 12.861 | 1.00 | 20.40 |
| ATOM | 2657 | CD1 | PHE | 387 | 1.653 | 18.573 | 12.479 | 1.00 | 21.35 |
| ATOM | 2658 | CD2 | PHE | 387 | 3.457 | 16.992 | 12.545 | 1.00 | 21.11 |
| ATOM | 2659 | CE1 | PHE | 387 | 0.863 | 17.651 | 11.789 | 1.00 | 22.07 |
| ATOM | 2660 | CE2 | PHE | 387 | 2.681 | 16.064 | 11.858 | 1.00 | 19.74 |
| ATOM | 2661 | CZ | PHE | 387 | 1.377 | 16.394 | 11.478 | 1.00 | 21.80 |
| ATOM | 2662 | C | PHE | 387 | 2.687 | 18.571 | 15.741 | 1.00 | 18.09 |
| ATOM | 2663 | O | PHE | 387 | 3.435 | 17.728 | 16.243 | 1.00 | 17.02 |
| ATOM | 2664 | N | MET | 388 | 1.363 | 18.562 | 15.881 | 1.00 | 17.04 |
| ATOM | 2665 | CA | MET | 388 | 0.615 | 17.528 | 16.589 | 1.00 | 18.41 |
| ATOM | 2666 | CB | MET | 388 | 0.742 | 16.197 | 15.839 | 1.00 | 19.83 |
| ATOM | 2667 | CG | MET | 388 | −0.430 | 15.255 | 16.044 | 1.00 | 19.70 |
| ATOM | 2668 | SD | MET | 388 | −1.962 | 15.929 | 15.362 | 1.00 | 19.18 |
| ATOM | 2669 | CE | MET | 388 | −1.899 | 15.299 | 13.685 | 1.00 | 23.05 |
| ATOM | 2670 | C | MET | 388 | 0.986 | 17.332 | 18.062 | 1.00 | 18.34 |
| ATOM | 2671 | O | MET | 388 | 0.779 | 16.254 | 18.622 | 1.00 | 19.91 |
| ATOM | 2672 | N | ALA | 389 | 1.520 | 18.376 | 18.688 | 1.00 | 17.11 |
| ATOM | 2673 | CA | ALA | 389 | 1.896 | 18.314 | 20.099 | 1.00 | 17.62 |
| ATOM | 2674 | CB | ALA | 389 | 3.178 | 19.124 | 20.345 | 1.00 | 16.15 |
| ATOM | 2675 | C | ALA | 389 | 0.764 | 18.839 | 20.987 | 1.00 | 17.04 |
| ATOM | 2676 | O | ALA | 389 | 0.893 | 18.891 | 22.211 | 1.00 | 17.48 |
| ATOM | 2677 | N | TYR | 390 | −0.343 | 19.231 | 20.367 | 1.00 | 17.06 |
| ATOM | 2678 | CA | TYR | 390 | −1.496 | 19.727 | 21.111 | 1.00 | 18.54 |
| ATOM | 2679 | CB | TYR | 390 | −1.422 | 21.261 | 21.266 | 1.00 | 17.65 |
| ATOM | 2680 | CG | TYR | 390 | −1.128 | 22.020 | 19.987 | 1.00 | 18.84 |
| ATOM | 2681 | CD1 | TYR | 390 | −2.157 | 22.468 | 19.157 | 1.00 | 21.00 |
| ATOM | 2682 | CE1 | TYR | 390 | −1.881 | 23.124 | 17.950 | 1.00 | 21.33 |
| ATOM | 2683 | CD2 | TYR | 390 | 0.183 | 22.247 | 19.585 | 1.00 | 19.67 |
| ATOM | 2684 | CE2 | TYR | 390 | 0.471 | 22.895 | 18.390 | 1.00 | 22.68 |
| ATOM | 2685 | CZ | TYR | 390 | −0.566 | 23.329 | 17.579 | 1.00 | 21.64 |
| ATOM | 2686 | OH | TYR | 390 | −0.262 | 23.963 | 16.394 | 1.00 | 23.80 |
| ATOM | 2687 | C | TYR | 390 | −2.790 | 19.291 | 20.437 | 1.00 | 18.26 |
| ATOM | 2688 | O | TYR | 390 | −3.765 | 20.039 | 20.376 | 1.00 | 18.45 |
| ATOM | 2689 | N | ALA | 391 | −2.780 | 18.050 | 19.949 | 1.00 | 18.00 |
| ATOM | 2690 | CA | ALA | 391 | −3.915 | 17.447 | 19.253 | 1.00 | 17.25 |
| ATOM | 2691 | CB | ALA | 391 | −3.497 | 16.109 | 18.656 | 1.00 | 18.23 |
| ATOM | 2692 | C | ALA | 391 | −5.112 | 17.258 | 20.177 | 1.00 | 17.88 |
| ATOM | 2693 | O | ALA | 391 | −6.250 | 17.147 | 19.719 | 1.00 | 18.08 |
| ATOM | 2694 | N | THR | 392 | −4.846 | 17.195 | 21.478 | 1.00 | 15.74 |
| ATOM | 2695 | CA | THR | 392 | −5.901 | 17.062 | 22.474 | 1.00 | 15.08 |
| ATOM | 2696 | CB | THR | 392 | −6.124 | 15.605 | 22.917 | 1.00 | 17.15 |
| ATOM | 2697 | OG1 | THR | 392 | −4.980 | 15.146 | 23.645 | 1.00 | 17.01 |
| ATOM | 2698 | CG2 | THR | 392 | −6.350 | 14.704 | 21.713 | 1.00 | 18.95 |
| ATOM | 2699 | C | THR | 392 | −5.445 | 17.857 | 23.682 | 1.00 | 14.74 |
| ATOM | 2700 | O | THR | 392 | −4.252 | 18.072 | 23.866 | 1.00 | 15.11 |
| ATOM | 2701 | N | PRO | 393 | −6.389 | 18.315 | 24.515 | 1.00 | 13.96 |
| ATOM | 2702 | CD | PRO | 393 | −7.851 | 18.291 | 24.353 | 1.00 | 15.31 |
| ATOM | 2703 | CA | PRO | 393 | −6.010 | 19.090 | 25.698 | 1.00 | 16.25 |
| ATOM | 2704 | CB | PRO | 393 | −7.349 | 19.309 | 26.398 | 1.00 | 15.99 |
| ATOM | 2705 | CG | PRO | 393 | −8.296 | 19.436 | 25.243 | 1.00 | 14.27 |
| ATOM | 2706 | C | PRO | 393 | −5.016 | 18.320 | 26.550 | 1.00 | 16.25 |
| ATOM | 2707 | O | PRO | 393 | −3.983 | 18.855 | 26.964 | 1.00 | 16.74 |
| ATOM | 2708 | N | GLU | 394 | −5.323 | 17.049 | 26.781 | 1.00 | 17.06 |
| ATOM | 2709 | CA | GLU | 394 | −4.474 | 16.188 | 27.586 | 1.00 | 18.79 |
| ATOM | 2710 | CB | GLU | 394 | −5.044 | 14.771 | 27.591 | 1.00 | 22.17 |
| ATOM | 2711 | CG | GLU | 394 | −4.455 | 13.860 | 28.630 | 1.00 | 29.13 |
| ATOM | 2712 | CD | GLU | 394 | −5.105 | 12.495 | 28.613 | 1.00 | 33.05 |
| ATOM | 2713 | OE1 | GLU | 394 | −4.687 | 11.644 | 27.797 | 1.00 | 35.69 |
| ATOM | 2714 | OE2 | GLU | 394 | −6.055 | 12.288 | 29.402 | 1.00 | 34.53 |
| ATOM | 2715 | C | GLU | 394 | −3.029 | 16.167 | 27.088 | 1.00 | 17.93 |
| ATOM | 2716 | O | GLU | 394 | −2.092 | 16.296 | 27.878 | 1.00 | 17.60 |
| ATOM | 2717 | N | GLN | 395 | −2.832 | 16.002 | 25.783 | 1.00 | 16.16 |
| ATOM | 2718 | CA | GLN | 395 | −1.469 | 15.973 | 25.261 | 1.00 | 18.13 |
| ATOM | 2719 | CB | GLN | 395 | −1.451 | 15.465 | 23.819 | 1.00 | 19.78 |
| ATOM | 2720 | CG | GLN | 395 | −1.662 | 13.958 | 23.738 | 1.00 | 26.51 |
| ATOM | 2721 | CD | GLN | 395 | −1.756 | 13.447 | 22.320 | 1.00 | 27.20 |
| ATOM | 2722 | OE1 | GLN | 395 | −0.821 | 13.590 | 21.535 | 1.00 | 31.74 |
| ATOM | 2723 | NE2 | GLN | 395 | −2.889 | 12.836 | 21.986 | 1.00 | 31.16 |
| ATOM | 2724 | C | GLN | 395 | −0.812 | 17.342 | 25.348 | 1.00 | 16.31 |
| ATOM | 2725 | O | GLN | 395 | 0.394 | 17.448 | 25.560 | 1.00 | 15.19 |
| ATOM | 2726 | N | ALA | 396 | −1.611 | 18.389 | 25.172 | 1.00 | 15.71 |
| ATOM | 2727 | CA | ALA | 396 | −1.097 | 19.748 | 25.258 | 1.00 | 15.45 |
| ATOM | 2728 | CB | ALA | 396 | −2.203 | 20.754 | 24.941 | 1.00 | 14.95 |
| ATOM | 2729 | C | ALA | 396 | −0.552 | 19.995 | 26.665 | 1.00 | 15.89 |
| ATOM | 2730 | O | ALA | 396 | 0.535 | 20.548 | 26.832 | 1.00 | 15.31 |
| ATOM | 2731 | N | PHE | 397 | −1.306 | 19.581 | 27.679 | 1.00 | 17.14 |
| ATOM | 2732 | CA | PHE | 397 | −0.862 | 19.780 | 29.059 | 1.00 | 17.67 |
| ATOM | 2733 | CB | PHE | 397 | −1.855 | 19.179 | 30.062 | 1.00 | 17.74 |
| ATOM | 2734 | CG | PHE | 397 | −3.276 | 19.626 | 29.876 | 1.00 | 15.92 |
| ATOM | 2735 | CD1 | PHE | 397 | −3.571 | 20.887 | 29.368 | 1.00 | 17.98 |
| ATOM | 2736 | CD2 | PHE | 397 | −4.325 | 18.787 | 30.242 | 1.00 | 18.15 |
| ATOM | 2737 | CE1 | PHE | 397 | −4.891 | 21.311 | 29.222 | 1.00 | 20.21 |
| ATOM | 2738 | CE2 | PHE | 397 | −5.650 | 19.197 | 30.103 | 1.00 | 19.30 |
| ATOM | 2739 | CZ | PHE | 397 | −5.934 | 20.465 | 29.591 | 1.00 | 20.13 |
| ATOM | 2740 | C | PHE | 397 | 0.496 | 19.120 | 29.277 | 1.00 | 17.87 |
| ATOM | 2741 | O | PHE | 397 | 1.397 | 19.710 | 29.867 | 1.00 | 18.64 |
| ATOM | 2742 | N | GLU | 398 | 0.628 | 17.887 | 28.793 | 1.00 | 18.39 |
| ATOM | 2743 | CA | GLU | 398 | 1.853 | 17.109 | 28.942 | 1.00 | 19.60 |
| ATOM | 2744 | CB | GLU | 398 | 1.612 | 15.691 | 28.411 | 1.00 | 22.86 |
| ATOM | 2745 | CG | GLU | 398 | 2.689 | 14.663 | 28.722 | 1.00 | 27.33 |
| ATOM | 2746 | CD | GLU | 398 | 2.800 | 14.340 | 30.203 | 1.00 | 31.00 |
| ATOM | 2747 | OE1 | GLU | 398 | 1.840 | 14.603 | 30.958 | 1.00 | 32.52 |
| ATOM | 2748 | OE2 | GLU | 398 | 3.854 | 13.804 | 30.606 | 1.00 | 34.89 |
| ATOM | 2749 | C | GLU | 398 | 3.047 | 17.740 | 28.224 | 1.00 | 17.86 |
| ATOM | 2750 | O | GLU | 398 | 4.139 | 17.864 | 28.791 | 1.00 | 17.51 |
| ATOM | 2751 | N | ASN | 399 | 2.836 | 18.143 | 26.977 | 1.00 | 17.52 |
| ATOM | 2752 | CA | ASN | 399 | 3.903 | 18.750 | 26.184 | 1.00 | 17.00 |
| ATOM | 2753 | CB | ASN | 399 | 3.545 | 18.695 | 24.696 | 1.00 | 17.91 |
| ATOM | 2754 | CG | ASN | 399 | 3.554 | 17.274 | 24.171 | 1.00 | 17.94 |
| ATOM | 2755 | OD1 | ASN | 399 | 4.421 | 16.482 | 24.557 | 1.00 | 16.74 |
| ATOM | 2756 | ND2 | ASN | 399 | 2.614 | 16.939 | 23.297 | 1.00 | 14.86 |
| ATOM | 2757 | C | ASN | 399 | 4.248 | 20.174 | 26.598 | 1.00 | 16.90 |
| ATOM | 2758 | O | ASN | 399 | 5.426 | 20.560 | 26.582 | 1.00 | 14.68 |
| ATOM | 2759 | N | ALA | 400 | 3.231 | 20.958 | 26.953 | 1.00 | 17.41 |
| ATOM | 2760 | CA | ALA | 400 | 3.465 | 22.324 | 27.403 | 1.00 | 17.58 |
| ATOM | 2761 | CB | ALA | 400 | 2.136 | 23.038 | 27.668 | 1.00 | 17.90 |
| ATOM | 2762 | C | ALA | 400 | 4.272 | 22.249 | 28.694 | 1.00 | 17.52 |
| ATOM | 2763 | O | ALA | 400 | 5.199 | 23.028 | 28.902 | 1.00 | 18.40 |
| ATOM | 2764 | N | ALA | 401 | 3.925 | 21.298 | 29.558 | 1.00 | 18.00 |
| ATOM | 2765 | CA | ALA | 401 | 4.629 | 21.152 | 30.825 | 1.00 | 17.86 |
| ATOM | 2766 | CB | ALA | 401 | 3.953 | 20.085 | 31.698 | 1.00 | 17.24 |
| ATOM | 2767 | C | ALA | 401 | 6.087 | 20.800 | 30.599 | 1.00 | 17.50 |
| ATOM | 2768 | O | ALA | 401 | 6.966 | 21.243 | 31.341 | 1.00 | 17.32 |
| ATOM | 2769 | N | THR | 402 | 6.349 | 20.004 | 29.569 | 1.00 | 17.75 |
| ATOM | 2770 | CA | THR | 402 | 7.714 | 19.604 | 29.259 | 1.00 | 17.20 |
| ATOM | 2771 | CB | THR | 402 | 7.744 | 18.579 | 28.100 | 1.00 | 17.19 |
| ATOM | 2772 | OG1 | THR | 402 | 7.194 | 17.332 | 28.551 | 1.00 | 15.26 |
| ATOM | 2773 | CG2 | THR | 402 | 9.169 | 18.361 | 27.621 | 1.00 | 17.10 |
| ATOM | 2774 | C | THR | 402 | 8.576 | 20.807 | 28.883 | 1.00 | 18.60 |
| ATOM | 2775 | O | THR | 402 | 9.690 | 20.954 | 29.380 | 1.00 | 18.32 |
| ATOM | 2776 | N | VAL | 403 | 8.063 | 21.675 | 28.016 | 1.00 | 18.13 |
| ATOM | 2777 | CA | VAL | 403 | 8.845 | 22.830 | 27.603 | 1.00 | 19.86 |
| ATOM | 2778 | CB | VAL | 403 | 8.313 | 23.416 | 26.275 | 1.00 | 20.58 |
| ATOM | 2779 | CG1 | VAL | 403 | 9.411 | 24.207 | 25.600 | 1.00 | 25.39 |
| ATOM | 2780 | CG2 | VAL | 403 | 7.857 | 22.290 | 25.344 | 1.00 | 22.84 |
| ATOM | 2781 | C | VAL | 403 | 8.886 | 23.901 | 28.702 | 1.00 | 21.59 |
| ATOM | 2782 | O | VAL | 403 | 9.825 | 24.701 | 28.769 | 1.00 | 21.71 |
| ATOM | 2783 | N | MET | 404 | 7.870 | 23.909 | 29.563 | 1.00 | 21.27 |
| ATOM | 2784 | CA | MET | 404 | 7.819 | 24.849 | 30.682 | 1.00 | 23.55 |
| ATOM | 2785 | CB | MET | 404 | 6.442 | 24.813 | 31.354 | 1.00 | 26.11 |
| ATOM | 2786 | CG | MET | 404 | 5.312 | 25.363 | 30.498 | 1.00 | 30.04 |
| ATOM | 2787 | SD | MET | 404 | 5.514 | 27.112 | 30.150 | 1.00 | 34.43 |
| ATOM | 2788 | CE | MET | 404 | 4.846 | 27.830 | 31.675 | 1.00 | 33.59 |
| ATOM | 2789 | C | MET | 404 | 8.902 | 24.474 | 31.704 | 1.00 | 22.56 |
| ATOM | 2790 | O | MET | 404 | 9.649 | 25.335 | 32.168 | 1.00 | 23.06 |
| ATOM | 2791 | N | ARG | 405 | 8.988 | 23.190 | 32.053 | 1.00 | 20.96 |
| ATOM | 2792 | CA | ARG | 405 | 9.998 | 22.739 | 33.008 | 1.00 | 21.29 |
| ATOM | 2793 | CB | ARG | 405 | 9.816 | 21.254 | 33.365 | 1.00 | 19.72 |
| ATOM | 2794 | CG | ARG | 405 | 8.471 | 20.900 | 33.973 | 1.00 | 19.05 |
| ATOM | 2795 | CD | ARG | 405 | 8.506 | 19.570 | 34.721 | 1.00 | 18.65 |
| ATOM | 2796 | NE | ARG | 405 | 7.157 | 19.145 | 35.096 | 1.00 | 22.57 |
| ATOM | 2797 | CZ | ARG | 405 | 6.330 | 18.474 | 34.299 | 1.00 | 21.33 |
| ATOM | 2798 | NH1 | ARG | 405 | 6.717 | 18.130 | 33.075 | 1.00 | 23.18 |
| ATOM | 2799 | NH2 | ARG | 405 | 5.101 | 18.180 | 34.709 | 1.00 | 20.27 |
| ATOM | 2800 | C | ARG | 405 | 11.395 | 22.948 | 32.419 | 1.00 | 21.30 |
| ATOM | 2801 | O | ARG | 405 | 12.385 | 23.004 | 33.144 | 1.00 | 20.18 |
| ATOM | 2802 | N | ALA | 406 | 11.465 | 23.068 | 31.097 | 1.00 | 21.02 |
| ATOM | 2803 | CA | ALA | 406 | 12.734 | 23.272 | 30.421 | 1.00 | 21.90 |
| ATOM | 2804 | CB | ALA | 406 | 12.625 | 22.844 | 28.961 | 1.00 | 22.53 |
| ATOM | 2805 | C | ALA | 406 | 13.188 | 24.730 | 30.511 | 1.00 | 24.17 |
| ATOM | 2806 | O | ALA | 406 | 14.331 | 25.050 | 30.182 | 1.00 | 24.00 |
| ATOM | 2807 | N | GLY | 407 | 12.298 | 25.616 | 30.950 | 1.00 | 24.29 |
| ATOM | 2808 | CA | GLY | 407 | 12.671 | 27.017 | 31.083 | 1.00 | 27.67 |
| ATOM | 2809 | C | GLY | 407 | 11.739 | 28.021 | 30.429 | 1.00 | 27.96 |
| ATOM | 2810 | O | GLY | 407 | 11.841 | 29.225 | 30.672 | 1.00 | 27.55 |
| ATOM | 2811 | N | ALA | 408 | 10.827 | 27.536 | 29.595 | 1.00 | 28.22 |
| ATOM | 2812 | CA | ALA | 408 | 9.892 | 28.423 | 28.920 | 1.00 | 27.88 |
| ATOM | 2813 | CB | ALA | 408 | 9.114 | 27.654 | 27.861 | 1.00 | 29.62 |
| ATOM | 2814 | C | ALA | 408 | 8.928 | 29.058 | 29.915 | 1.00 | 28.28 |
| ATOM | 2815 | O | ALA | 408 | 8.602 | 28.462 | 30.944 | 1.00 | 27.83 |
| ATOM | 2816 | N | ASN | 409 | 8.480 | 30.271 | 29.602 | 1.00 | 27.03 |
| ATOM | 2817 | CA | ASN | 409 | 7.539 | 30.996 | 30.454 | 1.00 | 27.18 |
| ATOM | 2818 | CB | ASN | 409 | 7.971 | 32.453 | 30.632 | 1.00 | 29.08 |
| ATOM | 2819 | CG | ASN | 409 | 9.327 | 32.590 | 31.274 | 1.00 | 28.79 |
| ATOM | 2820 | OD1 | ASN | 409 | 9.552 | 32.124 | 32.394 | 1.00 | 28.82 |
| ATOM | 2821 | ND2 | ASN | 409 | 10.245 | 33.244 | 30.571 | 1.00 | 27.34 |
| ATOM | 2822 | C | ASN | 409 | 6.156 | 30.993 | 29.823 | 1.00 | 26.67 |
| ATOM | 2823 | O | ASN | 409 | 5.150 | 31.201 | 30.501 | 1.00 | 25.46 |
| ATOM | 2824 | N | MET | 410 | 6.114 | 30.767 | 28.515 | 1.00 | 24.91 |
| ATOM | 2825 | CA | MET | 410 | 4.853 | 30.758 | 27.781 | 1.00 | 24.64 |
| ATOM | 2826 | CB | MET | 410 | 4.549 | 32.173 | 27.279 | 1.00 | 26.21 |
| ATOM | 2827 | CG | MET | 410 | 3.244 | 32.339 | 26.516 | 1.00 | 28.39 |
| ATOM | 2828 | SD | MET | 410 | 3.036 | 34.056 | 25.925 | 1.00 | 30.91 |
| ATOM | 2829 | CE | MET | 410 | 1.737 | 34.676 | 27.032 | 1.00 | 31.25 |
| ATOM | 2830 | C | MET | 410 | 4.947 | 29.794 | 26.605 | 1.00 | 23.26 |
| ATOM | 2831 | O | MET | 410 | 6.039 | 29.499 | 26.125 | 1.00 | 21.44 |
| ATOM | 2832 | N | VAL | 411 | 3.796 | 29.313 | 26.151 | 1.00 | 22.20 |
| ATOM | 2833 | CA | VAL | 411 | 3.726 | 28.381 | 25.035 | 1.00 | 24.32 |
| ATOM | 2834 | CB | VAL | 411 | 3.045 | 27.060 | 25.482 | 1.00 | 25.61 |
| ATOM | 2835 | CG1 | VAL | 411 | 2.625 | 26.248 | 24.281 | 1.00 | 30.37 |
| ATOM | 2836 | CG2 | VAL | 411 | 3.998 | 26.257 | 26.354 | 1.00 | 25.91 |
| ATOM | 2837 | C | VAL | 411 | 2.930 | 28.978 | 23.875 | 1.00 | 23.06 |
| ATOM | 2838 | O | VAL | 411 | 1.933 | 29.670 | 24.087 | 1.00 | 23.99 |
| ATOM | 2839 | N | LYS | 412 | 3.372 | 28.714 | 22.649 | 1.00 | 22.84 |
| ATOM | 2840 | CA | LYS | 412 | 2.674 | 29.220 | 21.471 | 1.00 | 22.52 |
| ATOM | 2841 | CB | LYS | 412 | 3.616 | 30.080 | 20.611 | 1.00 | 22.89 |
| ATOM | 2842 | CG | LYS | 412 | 2.927 | 30.727 | 19.405 | 1.00 | 23.26 |
| ATOM | 2843 | CD | LYS | 412 | 3.725 | 31.898 | 18.838 | 1.00 | 22.53 |
| ATOM | 2844 | CE | LYS | 412 | 4.979 | 31.436 | 18.103 | 1.00 | 22.01 |
| ATOM | 2845 | NZ | LYS | 412 | 4.638 | 30.645 | 16.892 | 1.00 | 20.94 |
| ATOM | 2846 | C | LYS | 412 | 2.129 | 28.056 | 20.649 | 1.00 | 23.26 |
| ATOM | 2847 | O | LYS | 412 | 2.830 | 27.071 | 20.406 | 1.00 | 22.79 |
| ATOM | 2848 | N | ILE | 413 | 0.870 | 28.170 | 20.240 | 1.00 | 23.60 |
| ATOM | 2849 | CA | ILE | 413 | 0.212 | 27.138 | 19.437 | 1.00 | 26.66 |
| ATOM | 2850 | CB | ILE | 413 | −0.758 | 26.291 | 20.295 | 1.00 | 26.12 |
| ATOM | 2851 | CG2 | ILE | 413 | 0.028 | 25.460 | 21.299 | 1.00 | 26.91 |
| ATOM | 2852 | CG1 | ILE | 413 | −1.751 | 27.203 | 21.020 | 1.00 | 28.28 |
| ATOM | 2853 | CD1 | ILE | 413 | −2.796 | 26.446 | 21.830 | 1.00 | 24.14 |
| ATOM | 2854 | C | ILE | 413 | −0.580 | 27.747 | 18.282 | 1.00 | 27.66 |
| ATOM | 2855 | O | ILE | 413 | −1.152 | 28.829 | 18.414 | 1.00 | 28.22 |
| ATOM | 2856 | N | GLU | 414 | −0.615 | 27.037 | 17.158 | 1.00 | 29.05 |
| ATOM | 2857 | CA | GLU | 414 | −1.318 | 27.489 | 15.958 | 1.00 | 30.52 |
| ATOM | 2858 | CB | GLU | 414 | −0.569 | 27.025 | 14.707 | 1.00 | 31.51 |
| ATOM | 2859 | CG | GLU | 414 | 0.834 | 27.574 | 14.561 | 1.00 | 32.37 |
| ATOM | 2860 | CD | GLU | 414 | 1.593 | 26.923 | 13.416 | 1.00 | 33.72 |
| ATOM | 2861 | OE1 | GLU | 414 | 0.951 | 26.261 | 12.568 | 1.00 | 32.40 |
| ATOM | 2862 | OE2 | GLU | 414 | 2.832 | 27.082 | 13.356 | 1.00 | 32.46 |
| ATOM | 2863 | C | GLU | 414 | −2.743 | 26.961 | 15.887 | 1.00 | 32.06 |
| ATOM | 2864 | O | GLU | 414 | −2.997 | 25.800 | 16.201 | 1.00 | 33.94 |
| ATOM | 2865 | N | GLY | 415 | −3.672 | 27.809 | 15.458 | 1.00 | 32.73 |
| ATOM | 2866 | CA | GLY | 415 | −5.052 | 27.372 | 15.345 | 1.00 | 33.14 |
| ATOM | 2867 | C | GLY | 415 | −6.074 | 28.431 | 15.703 | 1.00 | 32.54 |
| ATOM | 2868 | O | GLY | 415 | −5.744 | 29.432 | 16.336 | 1.00 | 31.25 |
| ATOM | 2869 | N | GLY | 416 | −7.322 | 28.199 | 15.302 | 1.00 | 33.53 |
| ATOM | 2870 | CA | GLY | 416 | −8.386 | 29.148 | 15.586 | 1.00 | 34.28 |
| ATOM | 2871 | C | GLY | 416 | −9.369 | 28.688 | 16.650 | 1.00 | 35.11 |
| ATOM | 2872 | O | GLY | 416 | −8.976 | 28.275 | 17.741 | 1.00 | 35.72 |
| ATOM | 2873 | N | GLU | 417 | −10.656 | 28.762 | 16.324 | 1.00 | 34.22 |
| ATOM | 2874 | CA | GLU | 417 | −11.723 | 28.370 | 17.240 | 1.00 | 34.97 |
| ATOM | 2875 | CB | GLU | 417 | −13.076 | 28.448 | 16.527 | 1.00 | 38.06 |
| ATOM | 2876 | CG | GLU | 417 | −13.709 | 29.829 | 16.530 | 1.00 | 44.07 |
| ATOM | 2877 | CD | GLU | 417 | −14.416 | 30.149 | 17.835 | 1.00 | 46.62 |
| ATOM | 2878 | OE1 | GLU | 417 | −13.763 | 30.094 | 18.900 | 1.00 | 48.75 |
| ATOM | 2879 | OE2 | GLU | 417 | −15.628 | 30.457 | 17.793 | 1.00 | 48.44 |
| ATOM | 2880 | C | GLU | 417 | −11.576 | 26.986 | 17.861 | 1.00 | 33.06 |
| ATOM | 2881 | O | GLU | 417 | −11.974 | 26.778 | 19.008 | 1.00 | 31.36 |
| ATOM | 2882 | N | TRP | 418 | −11.011 | 26.041 | 17.115 | 1.00 | 31.17 |
| ATOM | 2883 | CA | TRP | 418 | −10.865 | 24.683 | 17.632 | 1.00 | 30.62 |
| ATOM | 2884 | CB | TRP | 418 | −10.427 | 23.710 | 16.526 | 1.00 | 29.57 |
| ATOM | 2885 | CG | TRP | 418 | −8.968 | 23.794 | 16.161 | 1.00 | 27.67 |
| ATOM | 2886 | CD2 | TRP | 418 | −7.909 | 22.968 | 16.666 | 1.00 | 27.03 |
| ATOM | 2887 | CE2 | TRP | 418 | −6.710 | 23.410 | 16.063 | 1.00 | 25.39 |
| ATOM | 2888 | CE3 | TRP | 418 | −7.856 | 21.901 | 17.575 | 1.00 | 25.44 |
| ATOM | 2889 | CD1 | TRP | 418 | −8.386 | 24.674 | 15.297 | 1.00 | 27.61 |
| ATOM | 2890 | NE1 | TRP | 418 | −7.030 | 24.449 | 15.231 | 1.00 | 25.85 |
| ATOM | 2891 | CZ2 | TRP | 418 | −5.471 | 22.817 | 16.333 | 1.00 | 24.66 |
| ATOM | 2892 | CZ3 | TRP | 418 | −6.618 | 21.313 | 17.843 | 1.00 | 24.48 |
| ATOM | 2893 | CH2 | TRP | 418 | −5.446 | 21.777 | 17.225 | 1.00 | 24.32 |
| ATOM | 2894 | C | TRP | 418 | −9.900 | 24.568 | 18.810 | 1.00 | 29.99 |
| ATOM | 2895 | O | TRP | 418 | −9.793 | 23.500 | 19.417 | 1.00 | 31.23 |
| ATOM | 2896 | N | LEU | 419 | −9.208 | 25.661 | 19.131 | 1.00 | 26.75 |
| ATOM | 2897 | CA | LEU | 419 | −8.244 | 25.677 | 20.235 | 1.00 | 26.72 |
| ATOM | 2898 | CB | LEU | 419 | −6.950 | 26.371 | 19.809 | 1.00 | 25.59 |
| ATOM | 2899 | CG | LEU | 419 | −6.010 | 25.613 | 18.872 | 1.00 | 26.52 |
| ATOM | 2900 | CD1 | LEU | 419 | −4.746 | 26.434 | 18.644 | 1.00 | 25.61 |
| ATOM | 2901 | CD2 | LEU | 419 | −5.659 | 24.268 | 19.488 | 1.00 | 23.92 |
| ATOM | 2902 | C | LEU | 419 | −8.755 | 26.355 | 21.498 | 1.00 | 26.45 |
| ATOM | 2903 | O | LEU | 419 | −8.051 | 26.412 | 22.507 | 1.00 | 23.45 |
| ATOM | 2904 | N | VAL | 420 | −9.976 | 26.871 | 21.442 | 1.00 | 25.42 |
| ATOM | 2905 | CA | VAL | 420 | −10.562 | 27.544 | 22.591 | 1.00 | 25.27 |
| ATOM | 2906 | CB | VAL | 420 | −12.053 | 27.865 | 22.340 | 1.00 | 25.27 |
| ATOM | 2907 | CG1 | VAL | 420 | −12.701 | 28.408 | 23.619 | 1.00 | 25.70 |
| ATOM | 2908 | CG2 | VAL | 420 | −12.171 | 28.891 | 21.220 | 1.00 | 25.05 |
| ATOM | 2909 | C | VAL | 420 | −10.434 | 26.701 | 23.857 | 1.00 | 24.44 |
| ATOM | 2910 | O | VAL | 420 | −9.785 | 27.109 | 24.819 | 1.00 | 24.50 |
| ATOM | 2911 | N | GLU | 421 | −11.041 | 25.520 | 23.842 | 1.00 | 23.25 |
| ATOM | 2912 | CA | GLU | 421 | −11.000 | 24.635 | 24.999 | 1.00 | 23.68 |
| ATOM | 2913 | CB | GLU | 421 | −11.659 | 23.301 | 24.654 | 1.00 | 26.62 |
| ATOM | 2914 | CG | GLU | 421 | −11.745 | 22.350 | 25.825 | 1.00 | 30.68 |
| ATOM | 2915 | CD | GLU | 421 | −12.603 | 21.141 | 25.526 | 1.00 | 32.30 |
| ATOM | 2916 | OE1 | GLU | 421 | −12.199 | 20.308 | 24.688 | 1.00 | 32.73 |
| ATOM | 2917 | OE2 | GLU | 421 | −13.692 | 21.036 | 26.129 | 1.00 | 36.59 |
| ATOM | 2918 | C | GLU | 421 | −9.576 | 24.393 | 25.513 | 1.00 | 22.94 |
| ATOM | 2919 | O | GLU | 421 | −9.320 | 24.490 | 26.711 | 1.00 | 21.48 |
| ATOM | 2920 | N | THR | 422 | −8.660 | 24.079 | 24.602 | 1.00 | 21.94 |
| ATOM | 2921 | CA | THR | 422 | −7.267 | 23.827 | 24.952 | 1.00 | 20.34 |
| ATOM | 2922 | CB | THR | 422 | −6.456 | 23.462 | 23.692 | 1.00 | 21.24 |
| ATOM | 2923 | OG1 | THR | 422 | −7.015 | 22.283 | 23.103 | 1.00 | 20.48 |
| ATOM | 2924 | CG2 | THR | 422 | −4.991 | 23.211 | 24.032 | 1.00 | 19.54 |
| ATOM | 2925 | C | THR | 422 | −6.634 | 25.042 | 25.625 | 1.00 | 21.57 |
| ATOM | 2926 | O | THR | 422 | −5.871 | 24.916 | 26.591 | 1.00 | 20.08 |
| ATOM | 2927 | N | VAL | 423 | −6.951 | 26.224 | 25.109 | 1.00 | 22.38 |
| ATOM | 2928 | CA | VAL | 423 | −6.420 | 27.453 | 25.675 | 1.00 | 23.54 |
| ATOM | 2929 | CB | VAL | 423 | −6.755 | 28.672 | 24.794 | 1.00 | 24.18 |
| ATOM | 2930 | CG1 | VAL | 423 | −6.307 | 29.955 | 25.497 | 1.00 | 24.10 |
| ATOM | 2931 | CG2 | VAL | 423 | −6.064 | 28.540 | 23.455 | 1.00 | 22.31 |
| ATOM | 2932 | C | VAL | 423 | −6.973 | 27.699 | 27.074 | 1.00 | 24.57 |
| ATOM | 2933 | O | VAL | 423 | −6.221 | 28.014 | 27.994 | 1.00 | 24.45 |
| ATOM | 2934 | N | GLN | 424 | −8.286 | 27.554 | 27.231 | 1.00 | 24.53 |
| ATOM | 2935 | CA | GLN | 424 | −8.910 | 27.776 | 28.529 | 1.00 | 25.29 |
| ATOM | 2936 | CB | GLN | 424 | −10.429 | 27.587 | 28.438 | 1.00 | 26.08 |
| ATOM | 2937 | CG | GLN | 424 | −11.088 | 28.337 | 27.289 | 1.00 | 29.30 |
| ATOM | 2938 | CD | GLN | 424 | −12.604 | 28.243 | 27.319 | 1.00 | 31.02 |
| ATOM | 2939 | OE1 | GLN | 424 | −13.171 | 27.155 | 27.423 | 1.00 | 27.95 |
| ATOM | 2940 | NE2 | GLN | 424 | −13.269 | 29.390 | 27.220 | 1.00 | 33.12 |
| ATOM | 2941 | C | GLN | 424 | −8.338 | 26.819 | 29.568 | 1.00 | 25.02 |
| ATOM | 2942 | O | GLN | 424 | −7.998 | 27.225 | 30.677 | 1.00 | 23.19 |
| ATOM | 2943 | N | MET | 425 | −8.216 | 25.549 | 29.204 | 1.00 | 21.87 |
| ATOM | 2944 | CA | MET | 425 | −7.703 | 24.556 | 30.135 | 1.00 | 22.36 |
| ATOM | 2945 | CB | MET | 425 | −8.003 | 23.155 | 29.609 | 1.00 | 22.27 |
| ATOM | 2946 | CG | MET | 425 | −9.484 | 22.889 | 29.484 | 1.00 | 23.95 |
| ATOM | 2947 | SD | MET | 425 | −9.848 | 21.203 | 29.002 | 1.00 | 24.90 |
| ATOM | 2948 | CE | MET | 425 | −9.831 | 20.377 | 30.583 | 1.00 | 28.11 |
| ATOM | 2949 | C | MET | 425 | −6.216 | 24.707 | 30.448 | 1.00 | 20.61 |
| ATOM | 2950 | O | MET | 425 | −5.791 | 24.450 | 31.571 | 1.00 | 20.01 |
| ATOM | 2951 | N | LEU | 426 | −5.426 | 25.122 | 29.463 | 1.00 | 20.23 |
| ATOM | 2952 | CA | LEU | 426 | −3.999 | 25.315 | 29.685 | 1.00 | 20.83 |
| ATOM | 2953 | CB | LEU | 426 | −3.291 | 25.634 | 28.366 | 1.00 | 18.83 |
| ATOM | 2954 | CG | LEU | 426 | −2.789 | 24.412 | 27.576 | 1.00 | 16.82 |
| ATOM | 2955 | CD1 | LEU | 426 | −2.321 | 24.828 | 26.188 | 1.00 | 18.65 |
| ATOM | 2956 | CD2 | LEU | 426 | −1.642 | 23.764 | 28.344 | 1.00 | 18.65 |
| ATOM | 2957 | C | LEU | 426 | −3.810 | 26.465 | 30.668 | 1.00 | 24.13 |
| ATOM | 2958 | O | LEU | 426 | −2.952 | 26.411 | 31.550 | 1.00 | 23.47 |
| ATOM | 2959 | N | THR | 427 | −4.631 | 27.498 | 30.505 | 1.00 | 27.30 |
| ATOM | 2960 | CA | THR | 427 | −4.594 | 28.681 | 31.357 | 1.00 | 31.01 |
| ATOM | 2961 | CB | THR | 427 | −5.694 | 29.685 | 30.957 | 1.00 | 31.69 |
| ATOM | 2962 | OG1 | THR | 427 | −5.486 | 30.107 | 29.605 | 1.00 | 34.44 |
| ATOM | 2963 | CG2 | THR | 427 | −5.665 | 30.903 | 31.868 | 1.00 | 34.25 |
| ATOM | 2964 | C | THR | 427 | −4.774 | 28.340 | 32.831 | 1.00 | 31.38 |
| ATOM | 2965 | O | THR | 427 | −3.902 | 28.627 | 33.646 | 1.00 | 31.24 |
| ATOM | 2966 | N | GLU | 428 | −5.905 | 27.727 | 33.172 | 1.00 | 32.66 |
| ATOM | 2967 | CA | GLU | 428 | −6.168 | 27.375 | 34.563 | 1.00 | 32.58 |
| ATOM | 2968 | CB | GLU | 428 | −7.614 | 26.899 | 34.741 | 1.00 | 33.84 |
| ATOM | 2969 | CG | GLU | 428 | −8.107 | 25.949 | 33.673 | 1.00 | 34.13 |
| ATOM | 2970 | CD | GLU | 428 | −9.482 | 25.377 | 33.995 | 1.00 | 34.03 |
| ATOM | 2971 | OE1 | GLU | 428 | −10.368 | 26.145 | 34.438 | 1.00 | 30.96 |
| ATOM | 2972 | OE2 | GLU | 428 | −9.673 | 24.162 | 33.793 | 1.00 | 31.63 |
| ATOM | 2973 | C | GLU | 428 | −5.206 | 26.339 | 35.134 | 1.00 | 32.23 |
| ATOM | 2974 | O | GLU | 428 | −5.269 | 26.022 | 36.320 | 1.00 | 32.97 |
| ATOM | 2975 | N | ARG | 429 | −4.314 | 25.816 | 34.296 | 1.00 | 31.00 |
| ATOM | 2976 | CA | ARG | 429 | −3.339 | 24.831 | 34.753 | 1.00 | 27.80 |
| ATOM | 2977 | CB | ARG | 429 | −3.372 | 23.594 | 33.844 | 1.00 | 26.67 |
| ATOM | 2978 | CG | ARG | 429 | −4.594 | 22.731 | 34.106 | 1.00 | 25.42 |
| ATOM | 2979 | CD | ARG | 429 | −4.832 | 21.643 | 33.064 | 1.00 | 21.31 |
| ATOM | 2980 | NE | ARG | 429 | −6.072 | 20.932 | 33.372 | 1.00 | 20.15 |
| ATOM | 2981 | CZ | ARG | 429 | −7.270 | 21.513 | 33.413 | 1.00 | 19.59 |
| ATOM | 2982 | NH1 | ARG | 429 | −7.396 | 22.805 | 33.157 | 1.00 | 19.16 |
| ATOM | 2983 | NH2 | ARG | 429 | −8.340 | 20.815 | 33.736 | 1.00 | 18.53 |
| ATOM | 2984 | C | ARG | 429 | −1.930 | 25.423 | 34.825 | 1.00 | 28.16 |
| ATOM | 2985 | O | ARG | 429 | −0.934 | 24.708 | 34.728 | 1.00 | 27.12 |
| ATOM | 2986 | N | ALA | 430 | −1.865 | 26.742 | 34.990 | 1.00 | 29.24 |
| ATOM | 2987 | CA | ALA | 430 | −0.600 | 27.464 | 35.109 | 1.00 | 29.49 |
| ATOM | 2988 | CB | ALA | 430 | 0.233 | 26.864 | 36.237 | 1.00 | 29.80 |
| ATOM | 2989 | C | ALA | 430 | 0.239 | 27.544 | 33.834 | 1.00 | 29.79 |
| ATOM | 2990 | O | ALA | 430 | 1.468 | 27.521 | 33.900 | 1.00 | 30.37 |
| ATOM | 2991 | N | VAL | 431 | −0.409 | 27.641 | 32.676 | 1.00 | 29.07 |
| ATOM | 2992 | CA | VAL | 431 | 0.332 | 27.738 | 31.420 | 1.00 | 27.53 |
| ATOM | 2993 | CB | VAL | 431 | 0.279 | 26.405 | 30.623 | 1.00 | 27.98 |
| ATOM | 2994 | CG1 | VAL | 431 | 1.073 | 26.545 | 29.327 | 1.00 | 27.45 |
| ATOM | 2995 | CG2 | VAL | 431 | 0.848 | 25.274 | 31.457 | 1.00 | 25.30 |
| ATOM | 2996 | C | VAL | 431 | −0.175 | 28.866 | 30.522 | 1.00 | 27.45 |
| ATOM | 2997 | O | VAL | 431 | −1.231 | 28.750 | 29.894 | 1.00 | 27.25 |
| ATOM | 2998 | N | PRO | 432 | 0.570 | 29.986 | 30.460 | 1.00 | 26.79 |
| ATOM | 2999 | CD | PRO | 432 | 1.831 | 30.287 | 31.155 | 1.00 | 26.11 |
| ATOM | 3000 | CA | PRO | 432 | 0.159 | 31.115 | 29.617 | 1.00 | 26.19 |
| ATOM | 3001 | CB | PRO | 432 | 1.135 | 32.218 | 30.017 | 1.00 | 27.09 |
| ATOM | 3002 | CG | PRO | 432 | 2.360 | 31.452 | 30.362 | 1.00 | 29.57 |
| ATOM | 3003 | C | PRO | 432 | 0.285 | 30.715 | 28.152 | 1.00 | 24.89 |
| ATOM | 3004 | O | PRO | 432 | 1.205 | 29.987 | 27.775 | 1.00 | 24.44 |
| ATOM | 3005 | N | VAL | 433 | −0.642 | 31.192 | 27.333 | 1.00 | 23.55 |
| ATOM | 3006 | CA | VAL | 433 | −0.653 | 30.841 | 25.924 | 1.00 | 22.49 |
| ATOM | 3007 | CB | VAL | 433 | −1.895 | 29.990 | 25.592 | 1.00 | 20.80 |
| ATOM | 3008 | CG1 | VAL | 433 | −1.915 | 29.640 | 24.111 | 1.00 | 21.86 |
| ATOM | 3009 | CG2 | VAL | 433 | −1.896 | 28.726 | 26.449 | 1.00 | 19.73 |
| ATOM | 3010 | C | VAL | 433 | −0.633 | 32.030 | 24.980 | 1.00 | 24.12 |
| ATOM | 3011 | O | VAL | 433 | −1.244 | 33.068 | 25.244 | 1.00 | 22.28 |
| ATOM | 3012 | N | CYS | 434 | 0.091 | 31.850 | 23.880 | 1.00 | 25.46 |
| ATOM | 3013 | CA | CYS | 434 | 0.206 | 32.841 | 22.820 | 1.00 | 26.20 |
| ATOM | 3014 | CB | CYS | 434 | 1.675 | 33.159 | 22.543 | 1.00 | 27.30 |
| ATOM | 3015 | SG | CYS | 434 | 1.936 | 34.229 | 21.110 | 1.00 | 29.19 |
| ATOM | 3016 | C | CYS | 434 | −0.416 | 32.197 | 21.585 | 1.00 | 27.10 |
| ATOM | 3017 | O | CYS | 434 | 0.010 | 31.123 | 21.165 | 1.00 | 27.48 |
| ATOM | 3018 | N | GLY | 435 | −1.428 | 32.842 | 21.019 | 1.00 | 25.59 |
| ATOM | 3019 | CA | GLY | 435 | −2.078 | 32.298 | 19.842 | 1.00 | 27.29 |
| ATOM | 3020 | C | GLY | 435 | −1.269 | 32.541 | 18.586 | 1.00 | 27.32 |
| ATOM | 3021 | O | GLY | 435 | −0.237 | 33.211 | 18.629 | 1.00 | 26.82 |
| ATOM | 3022 | N | HIS | 436 | −1.737 | 32.005 | 17.463 | 1.00 | 27.79 |
| ATOM | 3023 | CA | HIS | 436 | −1.037 | 32.164 | 16.195 | 1.00 | 28.13 |
| ATOM | 3024 | CB | HIS | 436 | 0.117 | 31.158 | 16.128 | 1.00 | 28.29 |
| ATOM | 3025 | CG | HIS | 436 | 1.044 | 31.362 | 14.970 | 1.00 | 29.67 |
| ATOM | 3026 | CD2 | HIS | 436 | 0.893 | 32.043 | 13.809 | 1.00 | 28.18 |
| ATOM | 3027 | ND1 | HIS | 436 | 2.298 | 30.792 | 14.918 | 1.00 | 28.22 |
| ATOM | 3028 | CE1 | HIS | 436 | 2.878 | 31.110 | 13.775 | 1.00 | 29.12 |
| ATOM | 3029 | NE2 | HIS | 436 | 2.046 | 31.868 | 13.083 | 1.00 | 27.13 |
| ATOM | 3030 | C | HIS | 436 | −2.008 | 31.950 | 15.039 | 1.00 | 29.59 |
| ATOM | 3031 | O | HIS | 436 | −2.356 | 30.817 | 14.708 | 1.00 | 27.47 |
| ATOM | 3032 | N | LEU | 437 | −2.442 | 33.052 | 14.428 | 1.00 | 30.59 |
| ATOM | 3033 | CA | LEU | 437 | −3.384 | 32.999 | 13.314 | 1.00 | 31.59 |
| ATOM | 3034 | CB | LEU | 437 | −4.632 | 33.823 | 13.643 | 1.00 | 31.44 |
| ATOM | 3035 | CG | LEU | 437 | −5.519 | 33.332 | 14.790 | 1.00 | 30.60 |
| ATOM | 3036 | CD1 | LEU | 437 | −6.611 | 34.351 | 15.065 | 1.00 | 30.46 |
| ATOM | 3037 | CD2 | LEU | 437 | −6.124 | 31.978 | 14.429 | 1.00 | 30.85 |
| ATOM | 3038 | C | LEU | 437 | −2.771 | 33.507 | 12.015 | 1.00 | 32.51 |
| ATOM | 3039 | O | LEU | 437 | −1.758 | 34.204 | 12.024 | 1.00 | 32.44 |
| ATOM | 3040 | N | GLY | 438 | −3.399 | 33.154 | 10.898 | 1.00 | 34.06 |
| ATOM | 3041 | CA | GLY | 438 | −2.905 | 33.587 | 9.606 | 1.00 | 35.62 |
| ATOM | 3042 | C | GLY | 438 | −2.283 | 32.452 | 8.825 | 1.00 | 37.37 |
| ATOM | 3043 | O | GLY | 438 | −2.896 | 31.400 | 8.641 | 1.00 | 37.17 |
| ATOM | 3044 | N | LEU | 439 | −1.055 | 32.659 | 8.366 | 1.00 | 38.61 |
| ATOM | 3045 | CA | LEU | 439 | −0.355 | 31.642 | 7.601 | 1.00 | 39.93 |
| ATOM | 3046 | CB | LEU | 439 | 0.623 | 32.310 | 6.630 | 1.00 | 42.55 |
| ATOM | 3047 | CG | LEU | 439 | 1.104 | 31.510 | 5.414 | 1.00 | 44.13 |
| ATOM | 3048 | CD1 | LEU | 439 | 1.964 | 32.410 | 4.533 | 1.00 | 45.83 |
| ATOM | 3049 | CD2 | LEU | 439 | 1.889 | 30.288 | 5.858 | 1.00 | 43.74 |
| ATOM | 3050 | C | LEU | 439 | 0.388 | 30.719 | 8.567 | 1.00 | 40.30 |
| ATOM | 3051 | O | LEU | 439 | 1.549 | 30.957 | 8.902 | 1.00 | 40.50 |
| ATOM | 3052 | N | THR | 440 | −0.302 | 29.675 | 9.020 | 1.00 | 39.46 |
| ATOM | 3053 | CA | THR | 440 | 0.267 | 28.700 | 9.947 | 1.00 | 38.93 |
| ATOM | 3054 | CB | THR | 440 | −0.847 | 27.967 | 10.717 | 1.00 | 39.18 |
| ATOM | 3055 | OG1 | THR | 440 | −1.763 | 27.374 | 9.787 | 1.00 | 38.96 |
| ATOM | 3056 | CG2 | THR | 440 | −1.600 | 28.938 | 11.612 | 1.00 | 37.57 |
| ATOM | 3057 | C | THR | 440 | 1.108 | 27.677 | 9.181 | 1.00 | 38.59 |
| ATOM | 3058 | O | THR | 440 | 0.572 | 26.785 | 8.522 | 1.00 | 38.96 |
| ATOM | 3059 | N | PRO | 441 | 2.444 | 27.791 | 9.270 | 1.00 | 37.45 |
| ATOM | 3060 | CD | PRO | 441 | 3.166 | 28.736 | 10.142 | 1.00 | 37.29 |
| ATOM | 3061 | CA | PRO | 441 | 3.378 | 26.891 | 8.586 | 1.00 | 36.21 |
| ATOM | 3062 | CB | PRO | 441 | 4.747 | 27.433 | 8.998 | 1.00 | 36.37 |
| ATOM | 3063 | CG | PRO | 441 | 4.485 | 28.045 | 10.332 | 1.00 | 39.17 |
| ATOM | 3064 | C | PRO | 441 | 3.205 | 25.400 | 8.882 | 1.00 | 34.87 |
| ATOM | 3065 | O | PRO | 441 | 3.548 | 24.558 | 8.050 | 1.00 | 33.70 |
| ATOM | 3066 | N | GLN | 442 | 2.677 | 25.069 | 10.058 | 1.00 | 33.93 |
| ATOM | 3067 | CA | GLN | 442 | 2.453 | 23.668 | 10.406 | 1.00 | 32.20 |
| ATOM | 3068 | CB | GLN | 442 | 1.991 | 23.542 | 11.863 | 1.00 | 31.74 |
| ATOM | 3069 | CG | GLN | 442 | 3.122 | 23.343 | 12.873 | 1.00 | 29.51 |
| ATOM | 3070 | CD | GLN | 442 | 2.644 | 23.472 | 14.312 | 1.00 | 29.21 |
| ATOM | 3071 | OE1 | GLN | 442 | 1.579 | 22.972 | 14.670 | 1.00 | 27.25 |
| ATOM | 3072 | NE2 | GLN | 442 | 3.436 | 24.136 | 15.145 | 1.00 | 28.44 |
| ATOM | 3073 | C | GLN | 442 | 1.406 | 23.052 | 9.472 | 1.00 | 31.45 |
| ATOM | 3074 | O | GLN | 442 | 1.423 | 21.848 | 9.215 | 1.00 | 30.71 |
| ATOM | 3075 | N | SER | 443 | 0.500 | 23.885 | 8.965 | 1.00 | 30.60 |
| ATOM | 3076 | CA | SER | 443 | −0.546 | 23.420 | 8.058 | 1.00 | 31.30 |
| ATOM | 3077 | CB | SER | 443 | −1.881 | 24.089 | 8.394 | 1.00 | 31.62 |
| ATOM | 3078 | OG | SER | 443 | −2.343 | 23.707 | 9.680 | 1.00 | 32.96 |
| ATOM | 3079 | C | SER | 443 | −0.188 | 23.711 | 6.603 | 1.00 | 30.60 |
| ATOM | 3080 | O | SER | 443 | −1.070 | 23.829 | 5.751 | 1.00 | 29.98 |
| ATOM | 3081 | N | VAL | 444 | 1.109 | 23.818 | 6.323 | 1.00 | 29.83 |
| ATOM | 3082 | CA | VAL | 444 | 1.590 | 24.101 | 4.970 | 1.00 | 29.28 |
| ATOM | 3083 | CB | VAL | 444 | 3.143 | 24.079 | 4.909 | 1.00 | 30.47 |
| ATOM | 3084 | CG1 | VAL | 444 | 3.670 | 22.706 | 5.307 | 1.00 | 28.85 |
| ATOM | 3085 | CG2 | VAL | 444 | 3.616 | 24.440 | 3.504 | 1.00 | 30.63 |
| ATOM | 3086 | C | VAL | 444 | 1.039 | 23.101 | 3.952 | 1.00 | 30.00 |
| ATOM | 3087 | O | VAL | 444 | 0.718 | 23.466 | 2.819 | 1.00 | 30.16 |
| ATOM | 3088 | N | ASN | 445 | 0.925 | 21.842 | 4.361 | 1.00 | 28.62 |
| ATOM | 3089 | CA | ASN | 445 | 0.412 | 20.804 | 3.478 | 1.00 | 27.90 |
| ATOM | 3090 | CB | ASN | 445 | 0.666 | 19.425 | 4.089 | 1.00 | 26.96 |
| ATOM | 3091 | CG | ASN | 445 | 2.141 | 19.128 | 4.245 | 1.00 | 28.30 |
| ATOM | 3092 | OD1 | ASN | 445 | 2.868 | 19.009 | 3.257 | 1.00 | 30.31 |
| ATOM | 3093 | ND2 | ASN | 445 | 2.598 | 19.023 | 5.488 | 1.00 | 28.01 |
| ATOM | 3094 | C | ASN | 445 | −1.073 | 20.991 | 3.203 | 1.00 | 27.73 |
| ATOM | 3095 | O | ASN | 445 | −1.575 | 20.566 | 2.165 | 1.00 | 25.69 |
| ATOM | 3096 | N | ILE | 446 | −1.767 | 21.632 | 4.138 | 1.00 | 29.28 |
| ATOM | 3097 | CA | ILE | 446 | −3.196 | 21.890 | 3.993 | 1.00 | 31.96 |
| ATOM | 3098 | CB | ILE | 446 | −3.836 | 22.319 | 5.335 | 1.00 | 31.98 |
| ATOM | 3099 | CG2 | ILE | 446 | −5.264 | 22.799 | 5.105 | 1.00 | 31.16 |
| ATOM | 3100 | CG1 | ILE | 446 | −3.814 | 21.153 | 6.331 | 1.00 | 30.13 |
| ATOM | 3101 | CD1 | ILE | 446 | −4.670 | 19.979 | 5.928 | 1.00 | 31.79 |
| ATOM | 3102 | C | ILE | 446 | −3.411 | 23.007 | 2.982 | 1.00 | 34.41 |
| ATOM | 3103 | O | ILE | 446 | −4.239 | 22.889 | 2.080 | 1.00 | 33.82 |
| ATOM | 3104 | N | PHE | 447 | −2.657 | 24.092 | 3.142 | 1.00 | 36.64 |
| ATOM | 3105 | CA | PHE | 447 | −2.763 | 25.245 | 2.249 | 1.00 | 39.97 |
| ATOM | 3106 | CB | PHE | 447 | −2.040 | 26.452 | 2.852 | 1.00 | 39.70 |
| ATOM | 3107 | CG | PHE | 447 | −2.516 | 26.826 | 4.228 | 1.00 | 40.98 |
| ATOM | 3108 | CD1 | PHE | 447 | −3.860 | 27.100 | 4.467 | 1.00 | 41.63 |
| ATOM | 3109 | CD2 | PHE | 447 | −1.613 | 26.931 | 5.282 | 1.00 | 41.49 |
| ATOM | 3110 | CE1 | PHE | 447 | −4.299 | 27.474 | 5.738 | 1.00 | 41.71 |
| ATOM | 3111 | CE2 | PHE | 447 | −2.040 | 27.304 | 6.557 | 1.00 | 41.93 |
| ATOM | 3112 | CZ | PHE | 447 | −3.388 | 27.577 | 6.785 | 1.00 | 41.96 |
| ATOM | 3113 | C | PHE | 447 | −2.170 | 24.948 | 0.874 | 1.00 | 42.08 |
| ATOM | 3114 | O | PHE | 447 | −2.536 | 25.579 | −0.121 | 1.00 | 42.68 |
| ATOM | 3115 | N | GLY | 448 | −1.251 | 23.987 | 0.831 | 1.00 | 43.71 |
| ATOM | 3116 | CA | GLY | 448 | −0.608 | 23.622 | −0.418 | 1.00 | 45.97 |
| ATOM | 3117 | C | GLY | 448 | 0.576 | 24.528 | −0.695 | 1.00 | 47.94 |
| ATOM | 3118 | O | GLY | 448 | 0.963 | 24.729 | −1.846 | 1.00 | 48.22 |
| ATOM | 3119 | N | GLY | 449 | 1.150 | 25.076 | 0.371 | 1.00 | 49.19 |
| ATOM | 3120 | CA | GLY | 449 | 2.285 | 25.969 | 0.239 | 1.00 | 50.57 |
| ATOM | 3121 | C | GLY | 449 | 2.149 | 27.142 | 1.191 | 1.00 | 51.89 |
| ATOM | 3122 | O | GLY | 449 | 1.263 | 27.148 | 2.047 | 1.00 | 51.79 |
| ATOM | 3123 | N | TYR | 450 | 3.020 | 28.137 | 1.048 | 1.00 | 53.06 |
| ATOM | 3124 | CA | TYR | 450 | 2.970 | 29.311 | 1.912 | 1.00 | 54.66 |
| ATOM | 3125 | CB | TYR | 450 | 4.375 | 29.674 | 2.397 | 1.00 | 54.86 |
| ATOM | 3126 | CG | TYR | 450 | 5.085 | 28.537 | 3.093 | 1.00 | 55.77 |
| ATOM | 3127 | CD1 | TYR | 450 | 5.758 | 27.559 | 2.362 | 1.00 | 55.77 |
| ATOM | 3128 | CE1 | TYR | 450 | 6.387 | 26.490 | 2.996 | 1.00 | 56.62 |
| ATOM | 3129 | CD2 | TYR | 450 | 5.057 | 28.420 | 4.482 | 1.00 | 55.92 |
| ATOM | 3130 | CE2 | TYR | 450 | 5.681 | 27.354 | 5.128 | 1.00 | 56.38 |
| ATOM | 3131 | CZ | TYR | 450 | 6.344 | 26.392 | 4.378 | 1.00 | 56.70 |
| ATOM | 3132 | OH | TYR | 450 | 6.958 | 25.329 | 5.008 | 1.00 | 56.18 |
| ATOM | 3133 | C | TYR | 450 | 2.346 | 30.493 | 1.178 | 1.00 | 55.36 |
| ATOM | 3134 | O | TYR | 450 | 3.036 | 31.265 | 0.513 | 1.00 | 55.48 |
| ATOM | 3135 | N | LYS | 451 | 1.031 | 30.625 | 1.311 | 1.00 | 56.14 |
| ATOM | 3136 | CA | LYS | 451 | 0.291 | 31.695 | 0.659 | 1.00 | 56.56 |
| ATOM | 3137 | CB | LYS | 451 | −0.833 | 31.099 | −0.189 | 1.00 | 57.29 |
| ATOM | 3138 | CG | LYS | 451 | −0.410 | 29.887 | −1.008 | 1.00 | 57.70 |
| ATOM | 3139 | CD | LYS | 451 | −1.613 | 29.188 | −1.621 | 1.00 | 58.82 |
| ATOM | 3140 | CE | LYS | 451 | −1.232 | 27.840 | −2.215 | 1.00 | 58.93 |
| ATOM | 3141 | NZ | LYS | 451 | −2.426 | 27.115 | −2.731 | 1.00 | 58.99 |
| ATOM | 3142 | C | LYS | 451 | −0.296 | 32.626 | 1.714 | 1.00 | 56.60 |
| ATOM | 3143 | O | LYS | 451 | −0.542 | 32.216 | 2.849 | 1.00 | 56.17 |
| ATOM | 3144 | N | VAL | 452 | −0.521 | 33.879 | 1.333 | 1.00 | 56.36 |
| ATOM | 3145 | CA | VAL | 452 | −1.079 | 34.868 | 2.245 | 1.00 | 56.35 |
| ATOM | 3146 | CB | VAL | 452 | −0.945 | 36.297 | 1.667 | 1.00 | 56.62 |
| ATOM | 3147 | CG1 | VAL | 452 | −1.394 | 37.322 | 2.696 | 1.00 | 56.98 |
| ATOM | 3148 | CG2 | VAL | 452 | 0.491 | 36.553 | 1.247 | 1.00 | 56.04 |
| ATOM | 3149 | C | VAL | 452 | −2.557 | 34.591 | 2.514 | 1.00 | 56.29 |
| ATOM | 3150 | O | VAL | 452 | −3.383 | 34.631 | 1.600 | 1.00 | 56.01 |
| ATOM | 3151 | N | GLN | 453 | −2.880 | 34.304 | 3.772 | 1.00 | 56.06 |
| ATOM | 3152 | CA | GLN | 453 | −4.258 | 34.036 | 4.166 | 1.00 | 56.41 |
| ATOM | 3153 | CB | GLN | 453 | −4.303 | 33.044 | 5.334 | 1.00 | 56.31 |
| ATOM | 3154 | CG | GLN | 453 | −4.304 | 31.574 | 4.926 | 1.00 | 56.61 |
| ATOM | 3155 | CD | GLN | 453 | −3.011 | 31.138 | 4.268 | 1.00 | 57.19 |
| ATOM | 3156 | OE1 | GLN | 453 | −1.934 | 31.255 | 4.854 | 1.00 | 57.65 |
| ATOM | 3157 | NE2 | GLN | 453 | −3.111 | 30.624 | 3.048 | 1.00 | 55.74 |
| ATOM | 3158 | C | GLN | 453 | −4.965 | 35.327 | 4.565 | 1.00 | 56.38 |
| ATOM | 3159 | O | GLN | 453 | −4.333 | 36.375 | 4.704 | 1.00 | 55.54 |
| ATOM | 3160 | N | GLY | 454 | −6.281 | 35.244 | 4.746 | 1.00 | 56.76 |
| ATOM | 3161 | CA | GLY | 454 | −7.053 | 36.413 | 5.127 | 1.00 | 57.88 |
| ATOM | 3162 | C | GLY | 454 | −7.627 | 37.165 | 3.940 | 1.00 | 58.46 |
| ATOM | 3163 | O | GLY | 454 | −8.382 | 38.120 | 4.115 | 1.00 | 58.43 |
| ATOM | 3164 | N | ARG | 455 | −7.267 | 36.735 | 2.733 | 1.00 | 59.53 |
| ATOM | 3165 | CA | ARG | 455 | −7.750 | 37.371 | 1.509 | 1.00 | 61.02 |
| ATOM | 3166 | CB | ARG | 455 | −7.081 | 36.737 | 0.283 | 1.00 | 61.22 |
| ATOM | 3167 | CG | ARG | 455 | −5.603 | 37.080 | 0.120 | 1.00 | 61.34 |
| ATOM | 3168 | CD | ARG | 455 | −5.416 | 38.532 | −0.305 | 1.00 | 61.58 |
| ATOM | 3169 | NE | ARG | 455 | −4.023 | 38.979 | −0.232 | 1.00 | 61.55 |
| ATOM | 3170 | CZ | ARG | 455 | −3.013 | 38.444 | −0.914 | 1.00 | 60.83 |
| ATOM | 3171 | NH1 | ARG | 455 | −3.224 | 37.425 | −1.736 | 1.00 | 60.33 |
| ATOM | 3172 | NH2 | ARG | 455 | −1.788 | 38.935 | −0.778 | 1.00 | 60.86 |
| ATOM | 3173 | C | ARG | 455 | −9.268 | 37.252 | 1.387 | 1.00 | 61.98 |
| ATOM | 3174 | O | ARG | 455 | −9.805 | 36.157 | 1.214 | 1.00 | 61.66 |
| ATOM | 3175 | N | GLY | 456 | −9.955 | 38.387 | 1.482 | 1.00 | 62.93 |
| ATOM | 3176 | CA | GLY | 456 | −11.402 | 38.386 | 1.379 | 1.00 | 64.13 |
| ATOM | 3177 | C | GLY | 456 | −12.084 | 38.769 | 2.678 | 1.00 | 65.08 |
| ATOM | 3178 | O | GLY | 456 | −11.445 | 38.843 | 3.728 | 1.00 | 65.12 |
| ATOM | 3179 | N | ASP | 457 | −13.388 | 39.016 | 2.606 | 1.00 | 65.45 |
| ATOM | 3180 | CA | ASP | 457 | −14.162 | 39.392 | 3.783 | 1.00 | 65.79 |
| ATOM | 3181 | CB | ASP | 457 | −15.457 | 40.094 | 3.365 | 1.00 | 66.87 |
| ATOM | 3182 | CG | ASP | 457 | −15.205 | 41.358 | 2.565 | 1.00 | 67.41 |
| ATOM | 3183 | OD1 | ASP | 457 | −14.544 | 42.277 | 3.095 | 1.00 | 67.49 |
| ATOM | 3184 | OD2 | ASP | 457 | −15.668 | 41.432 | 1.407 | 1.00 | 68.24 |
| ATOM | 3185 | C | ASP | 457 | −14.494 | 38.159 | 4.616 | 1.00 | 65.50 |
| ATOM | 3186 | O | ASP | 457 | −14.485 | 38.208 | 5.846 | 1.00 | 65.44 |
| ATOM | 3187 | N | GLU | 458 | −14.788 | 37.055 | 3.936 | 1.00 | 65.17 |
| ATOM | 3188 | CA | GLU | 458 | −15.122 | 35.808 | 4.611 | 1.00 | 64.76 |
| ATOM | 3189 | CB | GLU | 458 | −15.460 | 34.726 | 3.583 | 1.00 | 65.72 |
| ATOM | 3190 | CG | GLU | 458 | −15.856 | 33.392 | 4.194 | 1.00 | 67.79 |
| ATOM | 3191 | CD | GLU | 458 | −16.235 | 32.360 | 3.149 | 1.00 | 68.91 |
| ATOM | 3192 | OE1 | GLU | 458 | −17.201 | 32.605 | 2.394 | 1.00 | 69.70 |
| ATOM | 3193 | OE2 | GLU | 458 | −15.568 | 31.305 | 3.083 | 1.00 | 69.17 |
| ATOM | 3194 | C | GLU | 458 | −13.956 | 35.348 | 5.480 | 1.00 | 63.98 |
| ATOM | 3195 | O | GLU | 458 | −14.115 | 35.121 | 6.681 | 1.00 | 63.71 |
| ATOM | 3196 | N | ALA | 459 | −12.785 | 35.213 | 4.866 | 1.00 | 62.74 |
| ATOM | 3197 | CA | ALA | 459 | −11.588 | 34.787 | 5.581 | 1.00 | 61.25 |
| ATOM | 3198 | CB | ALA | 459 | −10.462 | 34.507 | 4.594 | 1.00 | 61.27 |
| ATOM | 3199 | C | ALA | 459 | −11.159 | 35.863 | 6.575 | 1.00 | 60.06 |
| ATOM | 3200 | O | ALA | 459 | −10.630 | 35.560 | 7.645 | 1.00 | 59.75 |
| ATOM | 3201 | N | GLY | 460 | −11.392 | 37.119 | 6.212 | 1.00 | 58.46 |
| ATOM | 3202 | CA | GLY | 460 | −11.029 | 38.218 | 7.083 | 1.00 | 57.13 |
| ATOM | 3203 | C | GLY | 460 | −11.808 | 38.207 | 8.384 | 1.00 | 56.78 |
| ATOM | 3204 | O | GLY | 460 | −11.220 | 38.275 | 9.463 | 1.00 | 56.93 |
| ATOM | 3205 | N | ASP | 461 | −13.132 | 38.118 | 8.290 | 1.00 | 55.32 |
| ATOM | 3206 | CA | ASP | 461 | −13.974 | 38.101 | 9.481 | 1.00 | 54.77 |
| ATOM | 3207 | CB | ASP | 461 | −15.455 | 38.211 | 9.099 | 1.00 | 55.32 |
| ATOM | 3208 | CG | ASP | 461 | −15.783 | 39.512 | 8.386 | 1.00 | 56.16 |
| ATOM | 3209 | OD1 | ASP | 461 | −15.440 | 40.593 | 8.913 | 1.00 | 56.40 |
| ATOM | 3210 | OD2 | ASP | 461 | −16.395 | 39.452 | 7.301 | 1.00 | 56.69 |
| ATOM | 3211 | C | ASP | 461 | −13.753 | 36.825 | 10.287 | 1.00 | 53.74 |
| ATOM | 3212 | O | ASP | 461 | −13.998 | 36.792 | 11.491 | 1.00 | 53.31 |
| ATOM | 3213 | N | GLN | 462 | −13.290 | 35.776 | 9.614 | 1.00 | 53.09 |
| ATOM | 3214 | CA | GLN | 462 | −13.034 | 34.500 | 10.268 | 1.00 | 52.15 |
| ATOM | 3215 | CB | GLN | 462 | −12.908 | 33.387 | 9.228 | 1.00 | 51.74 |
| ATOM | 3216 | CG | GLN | 462 | −12.684 | 32.009 | 9.826 | 1.00 | 51.57 |
| ATOM | 3217 | CD | GLN | 462 | −13.729 | 31.655 | 10.865 | 1.00 | 52.18 |
| ATOM | 3218 | OE1 | GLN | 462 | −14.930 | 31.725 | 10.604 | 1.00 | 51.90 |
| ATOM | 3219 | NE2 | GLN | 462 | −13.275 | 31.268 | 12.053 | 1.00 | 52.39 |
| ATOM | 3220 | C | GLN | 462 | −11.767 | 34.567 | 11.108 | 1.00 | 51.68 |
| ATOM | 3221 | O | GLN | 462 | −11.647 | 33.878 | 12.121 | 1.00 | 52.10 |
| ATOM | 3222 | N | LEU | 463 | −10.819 | 35.397 | 10.686 | 1.00 | 51.14 |
| ATOM | 3223 | CA | LEU | 463 | −9.567 | 35.552 | 11.419 | 1.00 | 50.31 |
| ATOM | 3224 | CB | LEU | 463 | −8.478 | 36.116 | 10.507 | 1.00 | 51.43 |
| ATOM | 3225 | CG | LEU | 463 | −8.036 | 35.227 | 9.343 | 1.00 | 52.52 |
| ATOM | 3226 | CD1 | LEU | 463 | −6.962 | 35.949 | 8.542 | 1.00 | 53.49 |
| ATOM | 3227 | CD2 | LEU | 463 | −7.505 | 33.901 | 9.872 | 1.00 | 53.21 |
| ATOM | 3228 | C | LEU | 463 | −9.769 | 36.480 | 12.603 | 1.00 | 49.29 |
| ATOM | 3229 | O | LEU | 463 | −9.162 | 36.295 | 13.656 | 1.00 | 49.00 |
| ATOM | 3230 | N | LEU | 464 | −10.622 | 37.483 | 12.424 | 1.00 | 48.83 |
| ATOM | 3231 | CA | LEU | 464 | −10.907 | 38.439 | 13.488 | 1.00 | 48.51 |
| ATOM | 3232 | CB | LEU | 464 | −11.724 | 39.613 | 12.939 | 1.00 | 48.82 |
| ATOM | 3233 | CG | LEU | 464 | −11.609 | 40.957 | 13.668 | 1.00 | 50.56 |
| ATOM | 3234 | CD1 | LEU | 464 | −12.492 | 41.976 | 12.960 | 1.00 | 50.81 |
| ATOM | 3235 | CD2 | LEU | 464 | −12.017 | 40.827 | 15.126 | 1.00 | 49.90 |
| ATOM | 3236 | C | LEU | 464 | −11.697 | 37.725 | 14.582 | 1.00 | 46.83 |
| ATOM | 3237 | O | LEU | 464 | −11.471 | 37.950 | 15.772 | 1.00 | 46.51 |
| ATOM | 3238 | N | SER | 465 | −12.626 | 36.867 | 14.165 | 1.00 | 45.83 |
| ATOM | 3239 | CA | SER | 465 | −13.452 | 36.104 | 15.095 | 1.00 | 44.66 |
| ATOM | 3240 | CB | SER | 465 | −14.506 | 35.295 | 14.335 | 1.00 | 45.80 |
| ATOM | 3241 | OG | SER | 465 | −15.284 | 34.505 | 15.225 | 1.00 | 45.26 |
| ATOM | 3242 | C | SER | 465 | −12.576 | 35.156 | 15.900 | 1.00 | 43.62 |
| ATOM | 3243 | O | SER | 465 | −12.702 | 35.072 | 17.119 | 1.00 | 43.30 |
| ATOM | 3244 | N | ASP | 466 | −11.700 | 34.435 | 15.205 | 1.00 | 41.72 |
| ATOM | 3245 | CA | ASP | 466 | −10.796 | 33.503 | 15.867 | 1.00 | 40.59 |
| ATOM | 3246 | CB | ASP | 466 | −9.941 | 32.742 | 14.847 | 1.00 | 40.56 |
| ATOM | 3247 | CG | ASP | 466 | −10.730 | 31.693 | 14.086 | 1.00 | 41.88 |
| ATOM | 3248 | OD1 | ASP | 466 | −11.651 | 31.083 | 14.674 | 1.00 | 43.44 |
| ATOM | 3249 | OD2 | ASP | 466 | −10.418 | 31.458 | 12.902 | 1.00 | 41.17 |
| ATOM | 3250 | C | ASP | 466 | −9.885 | 34.265 | 16.817 | 1.00 | 38.59 |
| ATOM | 3251 | O | ASP | 466 | −9.566 | 33.780 | 17.900 | 1.00 | 37.69 |
| ATOM | 3252 | N | ALA | 467 | −9.472 | 35.460 | 16.408 | 1.00 | 37.24 |
| ATOM | 3253 | CA | ALA | 467 | −8.596 | 36.291 | 17.226 | 1.00 | 36.59 |
| ATOM | 3254 | CB | ALA | 467 | −8.232 | 37.568 | 16.472 | 1.00 | 35.34 |
| ATOM | 3255 | C | ALA | 467 | −9.284 | 36.635 | 18.541 | 1.00 | 36.59 |
| ATOM | 3256 | O | ALA | 467 | −8.707 | 36.477 | 19.616 | 1.00 | 36.56 |
| ATOM | 3257 | N | LEU | 468 | −10.520 | 37.116 | 18.450 | 1.00 | 36.29 |
| ATOM | 3258 | CA | LEU | 468 | −11.283 | 37.465 | 19.643 | 1.00 | 36.13 |
| ATOM | 3259 | CB | LEU | 468 | −12.610 | 38.119 | 19.254 | 1.00 | 36.56 |
| ATOM | 3260 | CG | LEU | 468 | −12.551 | 39.555 | 18.732 | 1.00 | 38.68 |
| ATOM | 3261 | CD1 | LEU | 468 | −13.871 | 39.910 | 18.062 | 1.00 | 40.41 |
| ATOM | 3262 | CD2 | LEU | 468 | −12.248 | 40.501 | 19.884 | 1.00 | 38.41 |
| ATOM | 3263 | C | LEU | 468 | −11.559 | 36.204 | 20.452 | 1.00 | 35.67 |
| ATOM | 3264 | O | LEU | 468 | −11.565 | 36.232 | 21.683 | 1.00 | 36.76 |
| ATOM | 3265 | N | ALA | 469 | −11.788 | 35.099 | 19.747 | 1.00 | 34.59 |
| ATOM | 3266 | CA | ALA | 469 | −12.077 | 33.820 | 20.387 | 1.00 | 34.17 |
| ATOM | 3267 | CB | ALA | 469 | −12.370 | 32.768 | 19.332 | 1.00 | 33.45 |
| ATOM | 3268 | C | ALA | 469 | −10.917 | 33.370 | 21.269 | 1.00 | 34.41 |
| ATOM | 3269 | O | ALA | 469 | −11.101 | 33.073 | 22.453 | 1.00 | 33.82 |
| ATOM | 3270 | N | LEU | 470 | −9.725 | 33.315 | 20.685 | 1.00 | 33.79 |
| ATOM | 3271 | CA | LEU | 470 | −8.534 | 32.911 | 21.420 | 1.00 | 34.13 |
| ATOM | 3272 | CB | LEU | 470 | −7.297 | 32.981 | 20.516 | 1.00 | 33.55 |
| ATOM | 3273 | CG | LEU | 470 | −7.261 | 32.030 | 19.317 | 1.00 | 32.57 |
| ATOM | 3274 | CD1 | LEU | 470 | −6.006 | 32.282 | 18.490 | 1.00 | 32.93 |
| ATOM | 3275 | CD2 | LEU | 470 | −7.289 | 30.589 | 19.803 | 1.00 | 32.13 |
| ATOM | 3276 | C | LEU | 470 | −8.347 | 33.816 | 22.627 | 1.00 | 34.20 |
| ATOM | 3277 | O | LEU | 470 | −8.061 | 33.347 | 23.732 | 1.00 | 34.77 |
| ATOM | 3278 | N | GLU | 471 | −8.516 | 35.119 | 22.417 | 1.00 | 33.48 |
| ATOM | 3279 | CA | GLU | 471 | −8.373 | 36.085 | 23.499 | 1.00 | 33.79 |
| ATOM | 3280 | CB | GLU | 471 | −8.594 | 37.506 | 22.978 | 1.00 | 34.37 |
| ATOM | 3281 | CG | GLU | 471 | −8.617 | 38.553 | 24.080 | 1.00 | 35.68 |
| ATOM | 3282 | CD | GLU | 471 | −8.985 | 39.930 | 23.565 | 1.00 | 36.13 |
| ATOM | 3283 | OE1 | GLU | 471 | −10.028 | 40.055 | 22.881 | 1.00 | 36.43 |
| ATOM | 3284 | OE2 | GLU | 471 | −8.233 | 40.884 | 23.851 | 1.00 | 35.43 |
| ATOM | 3285 | C | GLU | 471 | −9.376 | 35.796 | 24.613 | 1.00 | 33.42 |
| ATOM | 3286 | O | GLU | 471 | −9.022 | 35.778 | 25.793 | 1.00 | 32.79 |
| ATOM | 3287 | N | ALA | 472 | −10.631 | 35.584 | 24.232 | 1.00 | 33.45 |
| ATOM | 3288 | CA | ALA | 472 | −11.672 | 35.291 | 25.209 | 1.00 | 32.67 |
| ATOM | 3289 | CB | ALA | 472 | −13.019 | 35.130 | 24.506 | 1.00 | 32.92 |
| ATOM | 3290 | C | ALA | 472 | −11.308 | 34.015 | 25.963 | 1.00 | 32.35 |
| ATOM | 3291 | O | ALA | 472 | −11.559 | 33.900 | 27.163 | 1.00 | 31.64 |
| ATOM | 3292 | N | ALA | 473 | −10.702 | 33.067 | 25.252 | 1.00 | 31.77 |
| ATOM | 3293 | CA | ALA | 473 | −10.297 | 31.790 | 25.840 | 1.00 | 32.34 |
| ATOM | 3294 | CB | ALA | 473 | −9.802 | 30.850 | 24.743 | 1.00 | 32.34 |
| ATOM | 3295 | C | ALA | 473 | −9.222 | 31.946 | 26.915 | 1.00 | 32.08 |
| ATOM | 3296 | O | ALA | 473 | −9.133 | 31.132 | 27.835 | 1.00 | 32.20 |
| ATOM | 3297 | N | GLY | 474 | −8.401 | 32.987 | 26.793 | 1.00 | 32.17 |
| ATOM | 3298 | CA | GLY | 474 | −7.352 | 33.217 | 27.774 | 1.00 | 32.43 |
| ATOM | 3299 | C | GLY | 474 | −5.991 | 33.553 | 27.182 | 1.00 | 33.77 |
| ATOM | 3300 | O | GLY | 474 | −5.027 | 33.776 | 27.916 | 1.00 | 32.28 |
| ATOM | 3301 | N | ALA | 475 | −5.905 | 33.589 | 25.856 | 1.00 | 34.78 |
| ATOM | 3302 | CA | ALA | 475 | −4.646 | 33.905 | 25.189 | 1.00 | 36.82 |
| ATOM | 3303 | CB | ALA | 475 | −4.820 | 33.850 | 23.679 | 1.00 | 37.72 |
| ATOM | 3304 | C | ALA | 475 | −4.170 | 35.289 | 25.608 | 1.00 | 38.74 |
| ATOM | 3305 | O | ALA | 475 | −4.896 | 36.276 | 25.466 | 1.00 | 38.36 |
| ATOM | 3306 | N | GLN | 476 | −2.947 | 35.354 | 26.124 | 1.00 | 39.30 |
| ATOM | 3307 | CA | GLN | 476 | −2.370 | 36.618 | 26.573 | 1.00 | 41.42 |
| ATOM | 3308 | CB | GLN | 476 | −1.484 | 36.378 | 27.798 | 1.00 | 42.23 |
| ATOM | 3309 | CG | GLN | 476 | −2.195 | 35.668 | 28.940 | 1.00 | 44.93 |
| ATOM | 3310 | CD | GLN | 476 | −1.408 | 35.724 | 30.234 | 1.00 | 46.68 |
| ATOM | 3311 | OE1 | GLN | 476 | −1.176 | 36.802 | 30.780 | 1.00 | 48.89 |
| ATOM | 3312 | NE2 | GLN | 476 | −0.992 | 34.564 | 30.732 | 1.00 | 47.84 |
| ATOM | 3313 | C | GLN | 476 | −1.561 | 37.296 | 25.468 | 1.00 | 41.29 |
| ATOM | 3314 | O | GLN | 476 | −1.171 | 38.459 | 25.590 | 1.00 | 40.99 |
| ATOM | 3315 | N | LEU | 477 | −1.320 | 36.558 | 24.389 | 1.00 | 40.91 |
| ATOM | 3316 | CA | LEU | 477 | −0.565 | 37.058 | 23.247 | 1.00 | 39.33 |
| ATOM | 3317 | CB | LEU | 477 | 0.915 | 36.716 | 23.395 | 1.00 | 41.45 |
| ATOM | 3318 | CG | LEU | 477 | 1.785 | 37.738 | 24.121 | 1.00 | 41.89 |
| ATOM | 3319 | CD1 | LEU | 477 | 3.142 | 37.133 | 24.420 | 1.00 | 42.32 |
| ATOM | 3320 | CD2 | LEU | 477 | 1.927 | 38.977 | 23.252 | 1.00 | 43.54 |
| ATOM | 3321 | C | LEU | 477 | −1.086 | 36.455 | 21.953 | 1.00 | 38.33 |
| ATOM | 3322 | O | LEU | 477 | −1.714 | 35.400 | 21.964 | 1.00 | 36.02 |
| ATOM | 3323 | N | LEU | 478 | −0.820 | 37.130 | 20.840 | 1.00 | 37.63 |
| ATOM | 3324 | CA | LEU | 478 | −1.258 | 36.659 | 19.533 | 1.00 | 38.87 |
| ATOM | 3325 | CB | LEU | 478 | −2.600 | 37.288 | 19.158 | 1.00 | 38.65 |
| ATOM | 3326 | CG | LEU | 478 | −3.100 | 36.978 | 17.748 | 1.00 | 40.05 |
| ATOM | 3327 | CD1 | LEU | 478 | −3.361 | 35.488 | 17.611 | 1.00 | 39.92 |
| ATOM | 3328 | CD2 | LEU | 478 | −4.369 | 37.771 | 17.467 | 1.00 | 40.39 |
| ATOM | 3329 | C | LEU | 478 | −0.227 | 37.002 | 18.465 | 1.00 | 38.90 |
| ATOM | 3330 | O | LEU | 478 | 0.424 | 38.047 | 18.528 | 1.00 | 39.77 |
| ATOM | 3331 | N | VAL | 479 | −0.084 | 36.120 | 17.483 | 1.00 | 38.07 |
| ATOM | 3332 | CA | VAL | 479 | 0.862 | 36.337 | 16.402 | 1.00 | 37.86 |
| ATOM | 3333 | CB | VAL | 479 | 2.033 | 35.332 | 16.478 | 1.00 | 37.71 |
| ATOM | 3334 | CG1 | VAL | 479 | 2.952 | 35.503 | 15.284 | 1.00 | 37.57 |
| ATOM | 3335 | CG2 | VAL | 479 | 2.808 | 35.537 | 17.771 | 1.00 | 37.24 |
| ATOM | 3336 | C | VAL | 479 | 0.174 | 36.200 | 15.049 | 1.00 | 39.03 |
| ATOM | 3337 | O | VAL | 479 | −0.453 | 35.176 | 14.760 | 1.00 | 37.57 |
| ATOM | 3338 | N | LEU | 480 | 0.282 | 37.246 | 14.231 | 1.00 | 38.25 |
| ATOM | 3339 | CA | LEU | 480 | −0.307 | 37.251 | 12.893 | 1.00 | 38.69 |
| ATOM | 3340 | CB | LEU | 480 | −1.029 | 38.574 | 12.622 | 1.00 | 39.15 |
| ATOM | 3341 | CG | LEU | 480 | −2.307 | 38.841 | 13.418 | 1.00 | 39.59 |
| ATOM | 3342 | CD1 | LEU | 480 | −2.851 | 40.220 | 13.071 | 1.00 | 39.86 |
| ATOM | 3343 | CD2 | LEU | 480 | −3.340 | 37.766 | 13.098 | 1.00 | 40.86 |
| ATOM | 3344 | C | LEU | 480 | 0.816 | 37.067 | 11.884 | 1.00 | 38.26 |
| ATOM | 3345 | O | LEU | 480 | 1.818 | 37.776 | 11.935 | 1.00 | 37.03 |
| ATOM | 3346 | N | GLU | 481 | 0.648 | 36.115 | 10.972 | 1.00 | 38.45 |
| ATOM | 3347 | CA | GLU | 481 | 1.670 | 35.846 | 9.967 | 1.00 | 38.65 |
| ATOM | 3348 | CB | GLU | 481 | 2.287 | 34.469 | 10.204 | 1.00 | 38.33 |
| ATOM | 3349 | CG | GLU | 481 | 3.587 | 34.243 | 9.454 | 1.00 | 39.77 |
| ATOM | 3350 | CD | GLU | 481 | 4.111 | 32.833 | 9.611 | 1.00 | 38.78 |
| ATOM | 3351 | OE1 | GLU | 481 | 4.045 | 32.300 | 10.741 | 1.00 | 36.25 |
| ATOM | 3352 | OE2 | GLU | 481 | 4.597 | 32.266 | 8.605 | 1.00 | 39.84 |
| ATOM | 3353 | C | GLU | 481 | 1.123 | 35.914 | 8.546 | 1.00 | 39.06 |
| ATOM | 3354 | O | GLU | 481 | 0.152 | 35.234 | 8.209 | 1.00 | 37.10 |
| ATOM | 3355 | N | CYS | 482 | 1.768 | 36.732 | 7.719 | 1.00 | 41.00 |
| ATOM | 3356 | CA | CYS | 482 | 1.384 | 36.918 | 6.323 | 1.00 | 42.86 |
| ATOM | 3357 | CB | CYS | 482 | 1.841 | 35.721 | 5.490 | 1.00 | 42.97 |
| ATOM | 3358 | SG | CYS | 482 | 3.640 | 35.526 | 5.444 | 1.00 | 44.40 |
| ATOM | 3359 | C | CYS | 482 | −0.110 | 37.139 | 6.142 | 1.00 | 43.47 |
| ATOM | 3360 | O | CYS | 482 | −0.829 | 36.260 | 5.664 | 1.00 | 44.42 |
| ATOM | 3361 | N | VAL | 483 | −0.564 | 38.327 | 6.524 | 1.00 | 45.25 |
| ATOM | 3362 | CA | VAL | 483 | −1.969 | 38.697 | 6.411 | 1.00 | 47.42 |
| ATOM | 3363 | CB | VAL | 483 | −2.682 | 38.573 | 7.783 | 1.00 | 47.80 |
| ATOM | 3364 | CG1 | VAL | 483 | −2.199 | 39.655 | 8.730 | 1.00 | 47.74 |
| ATOM | 3365 | CG2 | VAL | 483 | −4.182 | 38.643 | 7.603 | 1.00 | 48.49 |
| ATOM | 3366 | C | VAL | 483 | −2.053 | 40.143 | 5.915 | 1.00 | 48.82 |
| ATOM | 3367 | O | VAL | 483 | −1.243 | 40.987 | 6.304 | 1.00 | 49.32 |
| ATOM | 3368 | N | PRO | 484 | −3.027 | 40.449 | 5.042 | 1.00 | 49.61 |
| ATOM | 3369 | CD | PRO | 484 | −4.159 | 39.629 | 4.579 | 1.00 | 50.26 |
| ATOM | 3370 | CA | PRO | 484 | −3.143 | 41.822 | 4.541 | 1.00 | 49.54 |
| ATOM | 3371 | CB | PRO | 484 | −4.459 | 41.797 | 3.758 | 1.00 | 49.60 |
| ATOM | 3372 | CG | PRO | 484 | −5.229 | 40.670 | 4.391 | 1.00 | 50.57 |
| ATOM | 3373 | C | PRO | 484 | −3.138 | 42.850 | 5.666 | 1.00 | 49.40 |
| ATOM | 3374 | O | PRO | 484 | −3.852 | 42.703 | 6.656 | 1.00 | 49.27 |
| ATOM | 3375 | N | VAL | 485 | −2.317 | 43.884 | 5.504 | 1.00 | 49.36 |
| ATOM | 3376 | CA | VAL | 485 | −2.184 | 44.942 | 6.500 | 1.00 | 49.33 |
| ATOM | 3377 | CB | VAL | 485 | −1.436 | 46.156 | 5.915 | 1.00 | 48.81 |
| ATOM | 3378 | CG1 | VAL | 485 | −1.169 | 47.171 | 7.006 | 1.00 | 49.88 |
| ATOM | 3379 | CG2 | VAL | 485 | −0.134 | 45.707 | 5.273 | 1.00 | 49.01 |
| ATOM | 3380 | C | VAL | 485 | −3.532 | 45.415 | 7.034 | 1.00 | 50.17 |
| ATOM | 3381 | O | VAL | 485 | −3.732 | 45.505 | 8.248 | 1.00 | 49.86 |
| ATOM | 3382 | N | GLU | 486 | −4.454 | 45.715 | 6.124 | 1.00 | 50.05 |
| ATOM | 3383 | CA | GLU | 486 | −5.784 | 46.183 | 6.504 | 1.00 | 50.38 |
| ATOM | 3384 | CB | GLU | 486 | −6.644 | 46.421 | 5.254 | 1.00 | 51.37 |
| ATOM | 3385 | CG | GLU | 486 | −6.277 | 45.568 | 4.041 | 1.00 | 52.69 |
| ATOM | 3386 | CD | GLU | 486 | −5.076 | 46.115 | 3.277 | 1.00 | 53.84 |
| ATOM | 3387 | OE1 | GLU | 486 | −5.120 | 47.296 | 2.872 | 1.00 | 54.68 |
| ATOM | 3388 | OE2 | GLU | 486 | −4.096 | 45.365 | 3.074 | 1.00 | 52.54 |
| ATOM | 3389 | C | GLU | 486 | −6.503 | 45.228 | 7.456 | 1.00 | 50.03 |
| ATOM | 3390 | O | GLU | 486 | −7.304 | 45.656 | 8.292 | 1.00 | 48.95 |
| ATOM | 3391 | N | LEU | 487 | −6.215 | 43.936 | 7.330 | 1.00 | 49.68 |
| ATOM | 3392 | CA | LEU | 487 | −6.837 | 42.932 | 8.187 | 1.00 | 50.28 |
| ATOM | 3393 | CB | LEU | 487 | −6.709 | 41.543 | 7.553 | 1.00 | 51.28 |
| ATOM | 3394 | CG | LEU | 487 | −7.813 | 40.529 | 7.871 | 1.00 | 52.57 |
| ATOM | 3393 | CD1 | LEU | 487 | −7.551 | 39.250 | 7.099 | 1.00 | 53.26 |
| ATOM | 3396 | CD2 | LEU | 487 | −7.875 | 40.253 | 9.361 | 1.00 | 53.45 |
| ATOM | 3397 | C | LEU | 487 | −6.158 | 42.948 | 9.556 | 1.00 | 49.71 |
| ATOM | 3398 | O | LEU | 487 | −6.811 | 42.801 | 10.591 | 1.00 | 48.49 |
| ATOM | 3399 | N | ALA | 488 | −4.841 | 43.128 | 9.549 | 1.00 | 49.21 |
| ATOM | 3400 | CA | ALA | 488 | −4.071 | 43.177 | 10.784 | 1.00 | 48.39 |
| ATOM | 3401 | CB | ALA | 488 | −2.583 | 43.275 | 10.468 | 1.00 | 48.13 |
| ATOM | 3402 | C | ALA | 488 | −4.513 | 44.380 | 11.607 | 1.00 | 48.23 |
| ATOM | 3403 | O | ALA | 488 | −4.465 | 44.353 | 12.835 | 1.00 | 48.24 |
| ATOM | 3404 | N | LYS | 489 | −4.946 | 45.433 | 10.923 | 1.00 | 48.36 |
| ATOM | 3405 | CA | LYS | 489 | −5.399 | 46.646 | 11.593 | 1.00 | 48.70 |
| ATOM | 3406 | CB | LYS | 489 | −5.738 | 47.721 | 10.564 | 1.00 | 49.47 |
| ATOM | 3407 | CG | LYS | 489 | −4.615 | 48.018 | 9.591 | 1.00 | 51.46 |
| ATOM | 3408 | CD | LYS | 489 | −5.070 | 48.969 | 8.495 | 1.00 | 53.59 |
| ATOM | 3409 | CE | LYS | 489 | −3.995 | 49.127 | 7.431 | 1.00 | 54.03 |
| ATOM | 3410 | NZ | LYS | 489 | −4.460 | 49.959 | 6.292 | 1.00 | 54.51 |
| ATOM | 3411 | C | LYS | 489 | −6.626 | 46.357 | 12.450 | 1.00 | 48.17 |
| ATOM | 3412 | O | LYS | 489 | −6.667 | 46.723 | 13.623 | 1.00 | 47.72 |
| ATOM | 3413 | N | ARG | 490 | −7.627 | 45.704 | 11.863 | 1.00 | 48.64 |
| ATOM | 3414 | CA | ARG | 490 | −8.842 | 45.377 | 12.605 | 1.00 | 48.86 |
| ATOM | 3415 | CB | ARG | 490 | −9.846 | 44.622 | 11.725 | 1.00 | 49.59 |
| ATOM | 3416 | CG | ARG | 490 | −10.743 | 45.511 | 10.884 | 1.00 | 50.73 |
| ATOM | 3417 | CD | ARG | 490 | −12.089 | 44.836 | 10.629 | 1.00 | 51.13 |
| ATOM | 3418 | NE | ARG | 490 | −11.966 | 43.619 | 9.832 | 1.00 | 51.85 |
| ATOM | 3419 | CZ | ARG | 490 | −12.959 | 42.759 | 9.616 | 1.00 | 51.49 |
| ATOM | 3420 | NH1 | ARG | 490 | −14.157 | 42.977 | 10.141 | 1.00 | 51.44 |
| ATOM | 3421 | NH2 | ARG | 490 | −12.755 | 41.680 | 8.871 | 1.00 | 50.98 |
| ATOM | 3422 | C | ARG | 490 | −8.525 | 44.532 | 13.831 | 1.00 | 48.05 |
| ATOM | 3423 | O | ARG | 490 | −8.860 | 44.904 | 14.956 | 1.00 | 48.14 |
| ATOM | 3424 | N | ILE | 491 | −7.874 | 43.394 | 13.603 | 1.00 | 47.40 |
| ATOM | 3425 | CA | ILE | 491 | −7.509 | 42.477 | 14.679 | 1.00 | 46.20 |
| ATOM | 3426 | CB | ILE | 491 | −6.621 | 41.323 | 14.150 | 1.00 | 45.42 |
| ATOM | 3427 | CG2 | ILE | 491 | −6.168 | 40.440 | 15.304 | 1.00 | 45.08 |
| ATOM | 3428 | CG1 | ILE | 491 | −7.403 | 40.494 | 13.126 | 1.00 | 45.31 |
| ATOM | 3429 | CD1 | ILE | 491 | −6.608 | 39.364 | 12.503 | 1.00 | 45.50 |
| ATOM | 3430 | C | ILE | 491 | −6.777 | 43.188 | 15.811 | 1.00 | 45.67 |
| ATOM | 3431 | O | ILE | 491 | −7.119 | 43.022 | 16.982 | 1.00 | 45.67 |
| ATOM | 3432 | N | THR | 492 | −5.773 | 43.984 | 15.460 | 1.00 | 45.88 |
| ATOM | 3433 | CA | THR | 492 | −4.998 | 44.715 | 16.456 | 1.00 | 45.57 |
| ATOM | 3434 | CB | THR | 492 | −3.790 | 45.421 | 15.814 | 1.00 | 44.78 |
| ATOM | 3435 | OG1 | THR | 492 | −3.046 | 44.481 | 15.030 | 1.00 | 44.42 |
| ATOM | 3436 | CG2 | THR | 492 | −2.882 | 46.000 | 16.891 | 1.00 | 43.82 |
| ATOM | 3437 | C | THR | 492 | −5.849 | 45.768 | 17.155 | 1.00 | 46.83 |
| ATOM | 3438 | O | THR | 492 | −5.732 | 45.968 | 18.366 | 1.00 | 47.12 |
| ATOM | 3439 | N | GLU | 493 | −6.699 | 46.444 | 16.387 | 1.00 | 47.21 |
| ATOM | 3440 | CA | GLU | 493 | −7.568 | 47.483 | 16.934 | 1.00 | 48.40 |
| ATOM | 3441 | CB | GLU | 493 | −7.898 | 48.523 | 15.860 | 1.00 | 49.93 |
| ATOM | 3442 | CG | GLU | 493 | −6.713 | 49.354 | 15.407 | 1.00 | 52.65 |
| ATOM | 3443 | CD | GLU | 493 | −7.088 | 50.372 | 14.347 | 1.00 | 54.51 |
| ATOM | 3444 | OE1 | GLU | 493 | −7.493 | 49.957 | 13.238 | 1.00 | 55.78 |
| ATOM | 3445 | OE2 | GLU | 493 | −6.980 | 51.585 | 14.623 | 1.00 | 55.00 |
| ATOM | 3446 | C | GLU | 493 | −8.866 | 46.909 | 17.486 | 1.00 | 47.51 |
| ATOM | 3447 | O | GLU | 493 | −9.806 | 47.651 | 17.776 | 1.00 | 48.38 |
| ATOM | 3448 | N | ALA | 494 | −8.914 | 45.587 | 17.630 | 1.00 | 45.76 |
| ATOM | 3449 | CA | ALA | 494 | −10.103 | 44.916 | 18.143 | 1.00 | 43.41 |
| ATOM | 3450 | CB | ALA | 494 | −10.674 | 43.983 | 17.081 | 1.00 | 43.68 |
| ATOM | 3451 | C | ALA | 494 | −9.790 | 44.129 | 19.407 | 1.00 | 41.36 |
| ATOM | 3452 | O | ALA | 494 | −10.674 | 43.879 | 20.226 | 1.00 | 40.45 |
| ATOM | 3453 | N | LEU | 495 | −8.528 | 43.741 | 19.559 | 1.00 | 39.65 |
| ATOM | 3454 | CA | LEU | 495 | −8.103 | 42.974 | 20.723 | 1.00 | 37.83 |
| ATOM | 3455 | CB | LEU | 495 | −7.126 | 41.866 | 20.310 | 1.00 | 37.72 |
| ATOM | 3456 | CG | LEU | 495 | −7.606 | 40.812 | 19.305 | 1.00 | 38.81 |
| ATOM | 3457 | CD1 | LEU | 495 | −6.498 | 39.797 | 19.069 | 1.00 | 37.67 |
| ATOM | 3458 | CD2 | LEU | 495 | −8.851 | 40.121 | 19.825 | 1.00 | 39.75 |
| ATOM | 3459 | C | LEU | 495 | −7.446 | 43.851 | 21.780 | 1.00 | 37.16 |
| ATOM | 3460 | O | LEU | 495 | −6.866 | 44.895 | 21.473 | 1.00 | 35.11 |
| ATOM | 3461 | N | ALA | 496 | −7.542 | 43.414 | 23.031 | 1.00 | 36.75 |
| ATOM | 3462 | CA | ALA | 496 | −6.954 | 44.141 | 24.143 | 1.00 | 37.22 |
| ATOM | 3463 | CB | ALA | 496 | −7.780 | 43.927 | 25.399 | 1.00 | 36.82 |
| ATOM | 3464 | C | ALA | 496 | −5.527 | 43.656 | 24.360 | 1.00 | 37.76 |
| ATOM | 3465 | O | ALA | 496 | −4.656 | 44.427 | 24.754 | 1.00 | 38.66 |
| ATOM | 3466 | N | ILE | 497 | −5.291 | 42.374 | 24.097 | 1.00 | 38.35 |
| ATOM | 3467 | CA | ILE | 497 | −3.961 | 41.793 | 24.263 | 1.00 | 38.83 |
| ATOM | 3468 | CB | ILE | 497 | −4.015 | 40.246 | 24.284 | 1.00 | 37.92 |
| ATOM | 3469 | CG2 | ILE | 497 | −4.894 | 39.770 | 25.438 | 1.00 | 37.32 |
| ATOM | 3470 | CG1 | ILE | 497 | −4.558 | 39.728 | 22.950 | 1.00 | 38.42 |
| ATOM | 3471 | CD1 | ILE | 497 | −4.531 | 38.220 | 22.814 | 1.00 | 38.06 |
| ATOM | 3472 | C | ILE | 497 | −3.052 | 42.220 | 23.117 | 1.00 | 39.35 |
| ATOM | 3473 | O | ILE | 497 | −3.517 | 42.443 | 21.998 | 1.00 | 40.82 |
| ATOM | 3474 | N | PRO | 498 | −1.739 | 42.333 | 23.377 | 1.00 | 39.89 |
| ATOM | 3475 | CD | PRO | 498 | −1.048 | 42.058 | 24.648 | 1.00 | 38.22 |
| ATOM | 3476 | CA | PRO | 498 | −0.786 | 42.737 | 22.336 | 1.00 | 39.86 |
| ATOM | 3477 | CB | PRO | 498 | 0.541 | 42.807 | 23.093 | 1.00 | 40.07 |
| ATOM | 3478 | CG | PRO | 498 | 0.359 | 41.804 | 24.190 | 1.00 | 39.95 |
| ATOM | 3479 | C | PRO | 498 | −0.745 | 41.764 | 21.158 | 1.00 | 40.23 |
| ATOM | 3480 | O | PRO | 498 | −0.824 | 40.551 | 21.341 | 1.00 | 40.13 |
| ATOM | 3481 | N | VAL | 499 | −0.631 | 42.302 | 19.947 | 1.00 | 40.04 |
| ATOM | 3482 | CA | VAL | 499 | −0.588 | 41.475 | 18.746 | 1.00 | 39.96 |
| ATOM | 3483 | CB | VAL | 499 | −1.711 | 41.877 | 17.754 | 1.00 | 39.82 |
| ATOM | 3484 | CG1 | VAL | 499 | −1.678 | 40.978 | 16.527 | 1.00 | 39.71 |
| ATOM | 3485 | CG2 | VAL | 499 | −3.068 | 41.785 | 18.440 | 1.00 | 39.57 |
| ATOM | 3486 | C | VAL | 499 | 0.762 | 41.592 | 18.040 | 1.00 | 40.53 |
| ATOM | 3487 | O | VAL | 499 | 1.081 | 42.633 | 17.468 | 1.00 | 40.65 |
| ATOM | 3488 | N | ILE | 500 | 1.555 | 40.524 | 18.089 | 1.00 | 41.28 |
| ATOM | 3489 | CA | ILE | 500 | 2.865 | 40.509 | 17.444 | 1.00 | 41.57 |
| ATOM | 3490 | CB | ILE | 500 | 3.828 | 39.525 | 18.142 | 1.00 | 42.09 |
| ATOM | 3491 | CG2 | ILE | 500 | 5.176 | 39.522 | 17.429 | 1.00 | 42.77 |
| ATOM | 3492 | CG1 | ILE | 500 | 4.006 | 39.927 | 19.608 | 1.00 | 43.30 |
| ATOM | 3493 | CD1 | ILE | 500 | 4.799 | 38.930 | 20.434 | 1.00 | 43.71 |
| ATOM | 3494 | C | ILE | 500 | 2.695 | 40.090 | 15.990 | 1.00 | 41.61 |
| ATOM | 3495 | O | ILE | 500 | 2.071 | 39.068 | 15.693 | 1.00 | 40.85 |
| ATOM | 3496 | N | GLY | 501 | 3.254 | 40.881 | 15.081 | 1.00 | 40.82 |
| ATOM | 3497 | CA | GLY | 501 | 3.118 | 40.572 | 13.672 | 1.00 | 40.29 |
| ATOM | 3498 | C | GLY | 501 | 4.371 | 40.098 | 12.968 | 1.00 | 39.63 |
| ATOM | 3499 | O | GLY | 501 | 5.494 | 40.348 | 13.405 | 1.00 | 39.28 |
| ATOM | 3500 | N | ILE | 502 | 4.154 | 39.401 | 11.860 | 1.00 | 39.72 |
| ATOM | 3501 | CA | ILE | 502 | 5.226 | 38.875 | 11.032 | 1.00 | 41.34 |
| ATOM | 3502 | CB | ILE | 502 | 5.709 | 37.488 | 11.553 | 1.00 | 41.52 |
| ATOM | 3503 | CG2 | ILE | 502 | 4.522 | 36.582 | 11.830 | 1.00 | 42.33 |
| ATOM | 3504 | CG1 | ILE | 502 | 6.659 | 36.845 | 10.544 | 1.00 | 42.33 |
| ATOM | 3505 | CD1 | ILE | 502 | 7.958 | 37.584 | 10.375 | 1.00 | 43.95 |
| ATOM | 3506 | C | ILE | 502 | 4.676 | 38.766 | 9.609 | 1.00 | 42.06 |
| ATOM | 3507 | O | ILE | 502 | 4.029 | 37.782 | 9.245 | 1.00 | 42.10 |
| ATOM | 3508 | N | GLY | 503 | 4.922 | 39.801 | 8.812 | 1.00 | 43.11 |
| ATOM | 3509 | CA | GLY | 503 | 4.426 | 39.817 | 7.449 | 1.00 | 44.13 |
| ATOM | 3510 | C | GLY | 503 | 2.981 | 40.273 | 7.444 | 1.00 | 44.13 |
| ATOM | 3511 | O | GLY | 503 | 2.215 | 39.950 | 6.535 | 1.00 | 44.93 |
| ATOM | 3512 | N | ALA | 504 | 2.612 | 41.028 | 8.474 | 1.00 | 44.37 |
| ATOM | 3513 | CA | ALA | 504 | 1.255 | 41.540 | 8.610 | 1.00 | 45.44 |
| ATOM | 3514 | CB | ALA | 504 | 0.612 | 40.976 | 9.871 | 1.00 | 45.34 |
| ATOM | 3515 | C | ALA | 504 | 1.249 | 43.065 | 8.663 | 1.00 | 45.59 |
| ATOM | 3516 | O | ALA | 504 | 0.245 | 43.677 | 9.026 | 1.00 | 45.09 |
| ATOM | 3517 | N | GLY | 505 | 2.373 | 43.672 | 8.296 | 1.00 | 46.27 |
| ATOM | 3518 | CA | GLY | 505 | 2.465 | 45.119 | 8.316 | 1.00 | 47.16 |
| ATOM | 3519 | C | GLY | 505 | 2.955 | 45.644 | 9.652 | 1.00 | 47.80 |
| ATOM | 3520 | O | GLY | 505 | 3.187 | 44.870 | 10.581 | 1.00 | 46.72 |
| ATOM | 3521 | N | ASN | 506 | 3.106 | 46.963 | 9.749 | 1.00 | 47.19 |
| ATOM | 3522 | CA | ASN | 506 | 3.582 | 47.600 | 10.974 | 1.00 | 47.23 |
| ATOM | 3523 | CB | ASN | 506 | 4.561 | 48.725 | 10.624 | 1.00 | 47.68 |
| ATOM | 3524 | CG | ASN | 506 | 3.972 | 49.737 | 9.656 | 1.00 | 47.63 |
| ATOM | 3525 | OD1 | ASN | 506 | 4.678 | 50.613 | 9.156 | 1.00 | 49.34 |
| ATOM | 3526 | ND2 | ASN | 506 | 2.676 | 49.623 | 9.388 | 1.00 | 47.05 |
| ATOM | 3527 | C | ASN | 506 | 2.445 | 48.146 | 11.836 | 1.00 | 47.02 |
| ATOM | 3528 | O | ASN | 506 | 2.671 | 48.962 | 12.734 | 1.00 | 45.93 |
| ATOM | 3529 | N | VAL | 507 | 1.229 | 47.681 | 11.561 | 1.00 | 46.55 |
| ATOM | 3530 | CA | VAL | 507 | 0.046 | 48.106 | 12.300 | 1.00 | 46.94 |
| ATOM | 3531 | CB | VAL | 507 | −1.238 | 47.855 | 11.482 | 1.00 | 47.55 |
| ATOM | 3532 | CG1 | VAL | 507 | −2.447 | 48.398 | 12.228 | 1.00 | 48.40 |
| ATOM | 3533 | CG2 | VAL | 507 | −1.117 | 48.506 | 10.117 | 1.00 | 48.35 |
| ATOM | 3534 | C | VAL | 507 | −0.070 | 47.357 | 13.622 | 1.00 | 46.72 |
| ATOM | 3535 | O | VAL | 507 | −0.693 | 47.842 | 14.567 | 1.00 | 47.09 |
| ATOM | 3536 | N | THR | 508 | 0.531 | 46.171 | 13.683 | 1.00 | 46.59 |
| ATOM | 3537 | CA | THR | 508 | 0.494 | 45.355 | 14.896 | 1.00 | 46.09 |
| ATOM | 3538 | CB | THR | 508 | 1.109 | 43.955 | 14.653 | 1.00 | 46.09 |
| ATOM | 3539 | OG1 | THR | 508 | 2.438 | 44.091 | 14.138 | 1.00 | 46.20 |
| ATOM | 3540 | CG2 | THR | 508 | 0.264 | 43.166 | 13.658 | 1.00 | 45.82 |
| ATOM | 3541 | C | THR | 508 | 1.239 | 46.036 | 16.042 | 1.00 | 45.51 |
| ATOM | 3542 | O | THR | 508 | 2.017 | 46.964 | 15.823 | 1.00 | 45.43 |
| ATOM | 3543 | N | ASP | 509 | 0.993 | 45.572 | 17.263 | 1.00 | 44.71 |
| ATOM | 3544 | CA | ASP | 509 | 1.630 | 46.140 | 18.447 | 1.00 | 44.33 |
| ATOM | 3545 | CB | ASP | 509 | 0.940 | 45.627 | 19.713 | 1.00 | 43.80 |
| ATOM | 3546 | CG | ASP | 509 | −0.541 | 45.942 | 19.738 | 1.00 | 43.81 |
| ATOM | 3547 | OD1 | ASP | 509 | −0.899 | 47.139 | 19.730 | 1.00 | 41.97 |
| ATOM | 3548 | OD2 | ASP | 509 | −1.349 | 44.990 | 19.764 | 1.00 | 44.48 |
| ATOM | 3549 | C | ASP | 509 | 3.113 | 45.801 | 18.510 | 1.00 | 44.55 |
| ATOM | 3550 | O | ASP | 509 | 3.914 | 46.576 | 19.030 | 1.00 | 44.42 |
| ATOM | 3551 | N | GLY | 510 | 3.473 | 44.636 | 17.983 | 1.00 | 44.35 |
| ATOM | 3552 | CA | GLY | 510 | 4.864 | 44.225 | 18.001 | 1.00 | 44.15 |
| ATOM | 3553 | C | GLY | 510 | 5.269 | 43.479 | 16.749 | 1.00 | 44.41 |
| ATOM | 3554 | O | GLY | 510 | 4.445 | 43.221 | 15.870 | 1.00 | 45.25 |
| ATOM | 3555 | N | GLN | 511 | 6.547 | 43.129 | 16.667 | 1.00 | 43.48 |
| ATOM | 3556 | CA | GLN | 511 | 7.063 | 42.406 | 15.513 | 1.00 | 42.71 |
| ATOM | 3557 | CB | GLN | 511 | 7.893 | 43.342 | 14.625 | 1.00 | 42.63 |
| ATOM | 3558 | CG | GLN | 511 | 7.109 | 44.465 | 13.958 | 1.00 | 39.39 |
| ATOM | 3559 | CD | GLN | 511 | 6.088 | 43.957 | 12.959 | 1.00 | 38.03 |
| ATOM | 3560 | OE1 | GLN | 511 | 6.386 | 43.093 | 12.136 | 1.00 | 36.45 |
| ATOM | 3561 | NE2 | GLN | 511 | 4.878 | 44.502 | 13.017 | 1.00 | 37.24 |
| ATOM | 3562 | C | GLN | 511 | 7.930 | 41.238 | 15.959 | 1.00 | 43.26 |
| ATOM | 3563 | O | GLN | 511 | 8.402 | 41.197 | 17.094 | 1.00 | 42.66 |
| ATOM | 3564 | N | ILE | 512 | 8.130 | 40.287 | 15.055 | 1.00 | 43.65 |
| ATOM | 3565 | CA | ILE | 512 | 8.957 | 39.125 | 15.343 | 1.00 | 44.30 |
| ATOM | 3566 | CB | ILE | 512 | 8.159 | 38.052 | 16.124 | 1.00 | 43.90 |
| ATOM | 3567 | CG2 | ILE | 512 | 7.079 | 37.448 | 15.234 | 1.00 | 44.70 |
| ATOM | 3568 | CG1 | ILE | 512 | 9.108 | 36.962 | 16.627 | 1.00 | 43.78 |
| ATOM | 3569 | CD1 | ILE | 512 | 8.463 | 35.995 | 17.595 | 1.00 | 43.66 |
| ATOM | 3570 | C | ILE | 512 | 9.472 | 38.536 | 14.028 | 1.00 | 44.66 |
| ATOM | 3571 | O | ILE | 512 | 8.843 | 38.689 | 12.979 | 1.00 | 44.73 |
| ATOM | 3572 | N | LEU | 513 | 10.625 | 37.879 | 14.087 | 1.00 | 45.11 |
| ATOM | 3573 | CA | LEU | 513 | 11.215 | 37.274 | 12.903 | 1.00 | 45.41 |
| ATOM | 3574 | CB | LEU | 513 | 11.690 | 38.360 | 11.936 | 1.00 | 47.61 |
| ATOM | 3575 | CG | LEU | 513 | 11.333 | 38.180 | 10.457 | 1.00 | 49.48 |
| ATOM | 3576 | CD1 | LEU | 513 | 12.121 | 39.187 | 9.645 | 1.00 | 50.43 |
| ATOM | 3577 | CD2 | LEU | 513 | 11.651 | 36.759 | 9.988 | 1.00 | 50.20 |
| ATOM | 3578 | C | LEU | 513 | 12.400 | 36.396 | 13.288 | 1.00 | 44.78 |
| ATOM | 3579 | O | LEU | 513 | 13.020 | 36.594 | 14.337 | 1.00 | 44.20 |
| ATOM | 3580 | N | VAL | 514 | 12.714 | 35.428 | 12.432 | 1.00 | 43.32 |
| ATOM | 3581 | CA | VAL | 514 | 13.834 | 34.532 | 12.683 | 1.00 | 43.17 |
| ATOM | 3582 | CB | VAL | 514 | 13.914 | 33.413 | 11.623 | 1.00 | 43.69 |
| ATOM | 3583 | CG1 | VAL | 514 | 15.057 | 32.460 | 11.960 | 1.00 | 43.51 |
| ATOM | 3584 | CG2 | VAL | 514 | 12.594 | 32.669 | 11.554 | 1.00 | 43.46 |
| ATOM | 3585 | C | VAL | 514 | 15.124 | 35.342 | 12.638 | 1.00 | 42.43 |
| ATOM | 3586 | O | VAL | 514 | 15.377 | 36.069 | 11.679 | 1.00 | 42.48 |
| ATOM | 3587 | N | MET | 515 | 15.931 | 35.214 | 13.684 | 1.00 | 41.21 |
| ATOM | 3588 | CA | MET | 515 | 17.190 | 35.935 | 13.772 | 1.00 | 40.22 |
| ATOM | 3589 | CB | MET | 515 | 17.929 | 35.543 | 15.050 | 1.00 | 38.49 |
| ATOM | 3590 | CG | MET | 515 | 18.189 | 34.057 | 15.180 | 1.00 | 37.77 |
| ATOM | 3591 | SD | MET | 515 | 19.693 | 33.741 | 16.107 | 1.00 | 37.43 |
| ATOM | 3592 | CE | MET | 515 | 20.902 | 33.853 | 14.776 | 1.00 | 39.24 |
| ATOM | 3593 | C | MET | 515 | 18.086 | 35.669 | 12.569 | 1.00 | 40.31 |
| ATOM | 3594 | O | MET | 515 | 18.749 | 36.575 | 12.063 | 1.00 | 39.94 |
| ATOM | 3595 | N | HIS | 516 | 18.100 | 34.420 | 12.116 | 1.00 | 40.39 |
| ATOM | 3596 | CA | HIS | 516 | 18.916 | 34.011 | 10.978 | 1.00 | 41.60 |
| ATOM | 3597 | CB | HIS | 516 | 18.683 | 32.523 | 10.694 | 1.00 | 40.66 |
| ATOM | 3598 | CG | HIS | 516 | 19.230 | 31.619 | 11.755 | 1.00 | 39.06 |
| ATOM | 3599 | CD2 | HIS | 516 | 18.682 | 31.162 | 12.906 | 1.00 | 37.78 |
| ATOM | 3600 | ND1 | HIS | 516 | 20.517 | 31.128 | 11.725 | 1.00 | 38.08 |
| ATOM | 3601 | CE1 | HIS | 516 | 20.740 | 30.409 | 12.810 | 1.00 | 35.88 |
| ATOM | 3602 | NE2 | HIS | 516 | 19.642 | 30.415 | 13.544 | 1.00 | 37.95 |
| ATOM | 3603 | C | HIS | 516 | 18.657 | 34.836 | 9.718 | 1.00 | 42.74 |
| ATOM | 3604 | O | HIS | 516 | 19.558 | 35.025 | 8.898 | 1.00 | 42.29 |
| ATOM | 3605 | N | ASP | 517 | 17.429 | 35.323 | 9.567 | 1.00 | 43.70 |
| ATOM | 3606 | CA | ASP | 517 | 17.064 | 36.134 | 8.410 | 1.00 | 45.81 |
| ATOM | 3607 | CB | ASP | 517 | 15.640 | 35.807 | 7.953 | 1.00 | 45.49 |
| ATOM | 3608 | CG | ASP | 517 | 15.572 | 34.538 | 7.124 | 1.00 | 47.31 |
| ATOM | 3609 | OD1 | ASP | 517 | 16.287 | 34.456 | 6.104 | 1.00 | 47.95 |
| ATOM | 3610 | OD2 | ASP | 517 | 14.803 | 33.627 | 7.481 | 1.00 | 48.46 |
| ATOM | 3611 | C | ASP | 517 | 17.175 | 37.625 | 8.708 | 1.00 | 46.65 |
| ATOM | 3612 | O | ASP | 517 | 17.175 | 38.452 | 7.794 | 1.00 | 47.65 |
| ATOM | 3613 | N | ALA | 518 | 17.272 | 37.965 | 9.988 | 1.00 | 47.52 |
| ATOM | 3614 | CA | ALA | 518 | 17.379 | 39.358 | 10.404 | 1.00 | 48.26 |
| ATOM | 3615 | CB | ALA | 518 | 16.748 | 39.540 | 11.780 | 1.00 | 47.98 |
| ATOM | 3616 | C | ALA | 518 | 18.833 | 39.817 | 10.432 | 1.00 | 48.77 |
| ATOM | 3617 | O | ALA | 518 | 19.113 | 41.014 | 10.470 | 1.00 | 48.43 |
| ATOM | 3618 | N | PHE | 519 | 19.758 | 38.863 | 10.412 | 1.00 | 49.79 |
| ATOM | 3619 | CA | PHE | 519 | 21.178 | 39.193 | 10.437 | 1.00 | 50.88 |
| ATOM | 3620 | CB | PHE | 519 | 21.810 | 38.675 | 11.723 | 1.00 | 51.40 |
| ATOM | 3621 | CG | PHE | 519 | 21.076 | 39.092 | 12.957 | 1.00 | 52.43 |
| ATOM | 3622 | CD1 | PHE | 519 | 20.855 | 40.436 | 13.228 | 1.00 | 53.32 |
| ATOM | 3623 | CD2 | PHE | 519 | 20.594 | 38.141 | 13.845 | 1.00 | 53.65 |
| ATOM | 3624 | CE1 | PHE | 519 | 20.160 | 40.829 | 14.369 | 1.00 | 53.53 |
| ATOM | 3625 | CE2 | PHE | 519 | 19.899 | 38.522 | 14.988 | 1.00 | 54.19 |
| ATOM | 3626 | CZ | PHE | 519 | 19.682 | 39.869 | 15.251 | 1.00 | 53.89 |
| ATOM | 3627 | C | PHE | 519 | 21.915 | 38.622 | 9.234 | 1.00 | 51.31 |
| ATOM | 3628 | O | PHE | 519 | 23.130 | 38.413 | 9.277 | 1.00 | 51.70 |
| ATOM | 3629 | N | GLY | 520 | 21.168 | 38.369 | 8.165 | 1.00 | 51.46 |
| ATOM | 3630 | CA | GLY | 520 | 21.752 | 37.832 | 6.950 | 1.00 | 52.29 |
| ATOM | 3631 | C | GLY | 520 | 22.668 | 36.646 | 7.172 | 1.00 | 52.89 |
| ATOM | 3632 | O | GLY | 520 | 23.880 | 36.740 | 6.973 | 1.00 | 52.01 |
| ATOM | 3633 | N | ILE | 521 | 22.085 | 35.526 | 7.582 | 1.00 | 53.71 |
| ATOM | 3634 | CA | ILE | 521 | 22.844 | 34.309 | 7.830 | 1.00 | 54.45 |
| ATOM | 3635 | CB | ILE | 521 | 22.669 | 33.839 | 9.287 | 1.00 | 53.41 |
| ATOM | 3636 | CG2 | ILE | 521 | 23.483 | 32.580 | 9.531 | 1.00 | 52.98 |
| ATOM | 3637 | CG1 | ILE | 521 | 23.109 | 34.948 | 10.243 | 1.00 | 52.97 |
| ATOM | 3638 | CD1 | ILE | 521 | 22.872 | 34.629 | 11.702 | 1.00 | 51.81 |
| ATOM | 3639 | C | ILE | 521 | 22.375 | 33.201 | 6.894 | 1.00 | 55.91 |
| ATOM | 3640 | O | ILE | 521 | 23.163 | 32.358 | 6.467 | 1.00 | 56.51 |
| ATOM | 3641 | N | THR | 522 | 21.089 | 33.216 | 6.566 | 1.00 | 57.60 |
| ATOM | 3642 | CA | THR | 522 | 20.517 | 32.203 | 5.692 | 1.00 | 60.04 |
| ATOM | 3643 | CB | THR | 522 | 19.147 | 31.738 | 6.218 | 1.00 | 60.24 |
| ATOM | 3644 | OG1 | THR | 522 | 18.278 | 32.869 | 6.353 | 1.00 | 59.37 |
| ATOM | 3645 | CG2 | THR | 522 | 19.301 | 31.058 | 7.569 | 1.00 | 60.73 |
| ATOM | 3646 | C | THR | 522 | 20.355 | 32.683 | 4.252 | 1.00 | 61.62 |
| ATOM | 3647 | O | THR | 522 | 19.702 | 33.695 | 3.992 | 1.00 | 61.80 |
| ATOM | 3648 | N | GLY | 523 | 20.959 | 31.942 | 3.324 | 1.00 | 63.15 |
| ATOM | 3649 | CA | GLY | 523 | 20.881 | 32.276 | 1.911 | 1.00 | 65.08 |
| ATOM | 3650 | C | GLY | 523 | 20.874 | 33.762 | 1.601 | 1.00 | 66.40 |
| ATOM | 3651 | O | GLY | 523 | 21.492 | 34.563 | 2.305 | 1.00 | 66.60 |
| ATOM | 3652 | N | GLY | 524 | 20.170 | 34.128 | 0.536 | 1.00 | 66.95 |
| ATOM | 3653 | CA | GLY | 524 | 20.092 | 35.522 | 0.146 | 1.00 | 67.88 |
| ATOM | 3654 | C | GLY | 524 | 18.666 | 35.958 | −0.116 | 1.00 | 68.78 |
| ATOM | 3655 | O | GLY | 524 | 18.280 | 37.078 | 0.220 | 1.00 | 69.41 |
| ATOM | 3656 | N | HIS | 525 | 17.880 | 35.072 | −0.719 | 1.00 | 69.01 |
| ATOM | 3657 | CA | HIS | 525 | 16.487 | 35.376 | −1.023 | 1.00 | 69.66 |
| ATOM | 3658 | CB | HIS | 525 | 15.987 | 34.488 | −2.165 | 1.00 | 70.70 |
| ATOM | 3659 | CG | HIS | 525 | 16.435 | 34.939 | −3.520 | 1.00 | 71.89 |
| ATOM | 3660 | CD2 | HIS | 525 | 17.174 | 34.317 | −4.469 | 1.00 | 72.56 |
| ATOM | 3661 | ND1 | HIS | 525 | 16.105 | 36.172 | −4.039 | 1.00 | 72.33 |
| ATOM | 3662 | CE1 | HIS | 525 | 16.620 | 36.290 | −5.250 | 1.00 | 72.53 |
| ATOM | 3663 | NE2 | HIS | 525 | 17.273 | 35.178 | −5.535 | 1.00 | 72.71 |
| ATOM | 3664 | C | HIS | 525 | 15.578 | 35.208 | 0.191 | 1.00 | 69.38 |
| ATOM | 3665 | O | HIS | 525 | 14.651 | 34.398 | 0.177 | 1.00 | 69.45 |
| ATOM | 3666 | N | ILE | 526 | 15.849 | 35.979 | 1.240 | 1.00 | 68.69 |
| ATOM | 3667 | CA | ILE | 526 | 15.047 | 35.922 | 2.457 | 1.00 | 67.80 |
| ATOM | 3668 | CB | ILE | 526 | 15.558 | 36.927 | 3.512 | 1.00 | 67.79 |
| ATOM | 3669 | CG2 | ILE | 526 | 17.003 | 36.613 | 3.872 | 1.00 | 68.10 |
| ATOM | 3670 | CG1 | ILE | 526 | 15.431 | 38.354 | 2.974 | 1.00 | 67.42 |
| ATOM | 3671 | CD1 | ILE | 526 | 15.759 | 39.424 | 3.991 | 1.00 | 67.22 |
| ATOM | 3672 | C | ILE | 526 | 13.601 | 36.271 | 2.116 | 1.00 | 67.09 |
| ATOM | 3673 | O | ILE | 526 | 13.325 | 36.817 | 1.050 | 1.00 | 67.20 |
| ATOM | 3674 | N | PRO | 527 | 12.657 | 35.959 | 3.018 | 1.00 | 66.11 |
| ATOM | 3675 | CD | PRO | 527 | 12.814 | 35.365 | 4.356 | 1.00 | 66.02 |
| ATOM | 3676 | CA | PRO | 527 | 11.251 | 36.270 | 2.747 | 1.00 | 65.35 |
| ATOM | 3677 | CB | PRO | 527 | 10.532 | 35.712 | 3.974 | 1.00 | 65.35 |
| ATOM | 3678 | CG | PRO | 527 | 11.563 | 35.834 | 5.053 | 1.00 | 65.87 |
| ATOM | 3679 | C | PRO | 527 | 11.023 | 37.768 | 2.555 | 1.00 | 64.42 |
| ATOM | 3680 | O | PRO | 527 | 11.728 | 38.591 | 3.141 | 1.00 | 64.06 |
| ATOM | 3681 | N | LYS | 528 | 10.038 | 38.110 | 1.729 | 1.00 | 63.60 |
| ATOM | 3682 | CA | LYS | 528 | 9.719 | 39.506 | 1.440 | 1.00 | 62.63 |
| ATOM | 3683 | CB | LYS | 528 | 8.504 | 39.596 | 0.507 | 1.00 | 63.43 |
| ATOM | 3684 | CG | LYS | 528 | 8.568 | 38.689 | −0.715 | 1.00 | 64.83 |
| ATOM | 3685 | CD | LYS | 528 | 8.054 | 37.291 | −0.389 | 1.00 | 65.58 |
| ATOM | 3686 | CE | LYS | 528 | 8.237 | 36.336 | −1.559 | 1.00 | 65.50 |
| ATOM | 3687 | NZ | LYS | 528 | 9.678 | 36.076 | −1.831 | 1.00 | 65.11 |
| ATOM | 3688 | C | LYS | 528 | 9.443 | 40.317 | 2.702 | 1.00 | 61.12 |
| ATOM | 3689 | O | LYS | 528 | 9.874 | 41.465 | 2.815 | 1.00 | 60.81 |
| ATOM | 3690 | N | PHE | 529 | 8.726 | 39.715 | 3.648 | 1.00 | 59.49 |
| ATOM | 3691 | CA | PHE | 529 | 8.379 | 40.383 | 4.899 | 1.00 | 57.86 |
| ATOM | 3692 | CB | PHE | 529 | 7.258 | 39.611 | 5.608 | 1.00 | 57.20 |
| ATOM | 3693 | CG | PHE | 529 | 7.586 | 38.168 | 5.885 | 1.00 | 56.39 |
| ATOM | 3694 | CD1 | PHE | 529 | 8.487 | 37.820 | 6.887 | 1.00 | 55.45 |
| ATOM | 3695 | CD2 | PHE | 529 | 6.990 | 37.153 | 5.140 | 1.00 | 56.22 |
| ATOM | 3696 | CE1 | PHE | 529 | 8.787 | 36.483 | 7.146 | 1.00 | 55.50 |
| ATOM | 3697 | CE2 | PHE | 529 | 7.283 | 35.813 | 5.390 | 1.00 | 55.77 |
| ATOM | 3698 | CZ | PHE | 529 | 8.183 | 35.478 | 6.396 | 1.00 | 55.99 |
| ATOM | 3699 | C | PHE | 529 | 9.564 | 40.562 | 5.844 | 1.00 | 56.78 |
| ATOM | 3700 | O | PHE | 529 | 9.463 | 41.272 | 6.843 | 1.00 | 56.44 |
| ATOM | 3701 | N | ALA | 530 | 10.684 | 39.923 | 5.525 | 1.00 | 55.87 |
| ATOM | 3702 | CA | ALA | 530 | 11.878 | 40.015 | 6.357 | 1.00 | 55.61 |
| ATOM | 3703 | CB | ALA | 530 | 12.612 | 38.681 | 6.366 | 1.00 | 55.37 |
| ATOM | 3704 | C | ALA | 530 | 12.809 | 41.114 | 5.864 | 1.00 | 55.36 |
| ATOM | 3705 | O | ALA | 530 | 12.597 | 41.687 | 4.795 | 1.00 | 55.63 |
| ATOM | 3706 | N | LYS | 531 | 13.842 | 41.399 | 6.651 | 1.00 | 55.00 |
| ATOM | 3707 | CA | LYS | 531 | 14.816 | 42.425 | 6.301 | 1.00 | 54.83 |
| ATOM | 3708 | CB | LYS | 531 | 14.303 | 43.806 | 6.712 | 1.00 | 54.88 |
| ATOM | 3709 | CG | LYS | 531 | 15.283 | 44.932 | 6.428 | 1.00 | 55.59 |
| ATOM | 3710 | CD | LYS | 531 | 14.726 | 46.280 | 6.851 | 1.00 | 55.93 |
| ATOM | 3711 | CE | LYS | 531 | 15.716 | 47.397 | 6.560 | 1.00 | 56.02 |
| ATOM | 3712 | NZ | LYS | 531 | 15.170 | 48.729 | 6.933 | 1.00 | 55.93 |
| ATOM | 3713 | C | LYS | 531 | 16.161 | 42.166 | 6.970 | 1.00 | 54.84 |
| ATOM | 3714 | O | LYS | 531 | 16.220 | 41.840 | 8.154 | 1.00 | 54.41 |
| ATOM | 3715 | N | ASN | 532 | 17.237 | 42.315 | 6.203 | 1.00 | 54.78 |
| ATOM | 3716 | CA | ASN | 532 | 18.591 | 42.110 | 6.708 | 1.00 | 54.97 |
| ATOM | 3717 | CB | ASN | 532 | 19.544 | 41.805 | 5.548 | 1.00 | 54.87 |
| ATOM | 3718 | CG | ASN | 532 | 20.931 | 41.399 | 6.017 | 1.00 | 53.96 |
| ATOM | 3719 | OD1 | ASN | 532 | 21.469 | 41.963 | 6.970 | 1.00 | 54.05 |
| ATOM | 3720 | ND2 | ASN | 532 | 21.522 | 40.425 | 5.335 | 1.00 | 54.07 |
| ATOM | 3721 | C | ASN | 532 | 19.061 | 43.373 | 7.424 | 1.00 | 55.48 |
| ATOM | 3722 | O | ASN | 532 | 19.215 | 44.422 | 6.798 | 1.00 | 56.16 |
| ATOM | 3723 | N | PHE | 533 | 19.290 | 43.275 | 8.730 | 1.00 | 55.65 |
| ATOM | 3724 | CA | PHE | 533 | 19.744 | 44.425 | 9.503 | 1.00 | 56.07 |
| ATOM | 3725 | CB | PHE | 533 | 19.055 | 44.463 | 10.871 | 1.00 | 56.06 |
| ATOM | 3726 | CG | PHE | 533 | 17.607 | 44.863 | 10.812 | 1.00 | 56.39 |
| ATOM | 3727 | CD1 | PHE | 533 | 16.649 | 43.993 | 10.303 | 1.00 | 56.26 |
| ATOM | 3728 | CD2 | PHE | 533 | 17.204 | 46.124 | 11.245 | 1.00 | 56.64 |
| ATOM | 3729 | CE1 | PHE | 533 | 15.311 | 44.371 | 10.228 | 1.00 | 56.57 |
| ATOM | 3730 | CE2 | PHE | 533 | 15.868 | 46.511 | 11.173 | 1.00 | 56.36 |
| ATOM | 3731 | CZ | PHE | 533 | 14.921 | 45.634 | 10.663 | 1.00 | 56.79 |
| ATOM | 3732 | C | PHE | 533 | 21.255 | 44.446 | 9.695 | 1.00 | 56.80 |
| ATOM | 3733 | O | PHE | 533 | 21.808 | 45.424 | 10.199 | 1.00 | 56.46 |
| ATOM | 3734 | N | LEU | 534 | 21.920 | 43.367 | 9.297 | 1.00 | 57.79 |
| ATOM | 3735 | CA | LEU | 534 | 23.370 | 43.281 | 9.427 | 1.00 | 58.91 |
| ATOM | 3736 | CB | LEU | 534 | 23.811 | 41.820 | 9.535 | 1.00 | 58.06 |
| ATOM | 3737 | CG | LEU | 534 | 25.310 | 41.588 | 9.748 | 1.00 | 57.55 |
| ATOM | 3738 | CD1 | LEU | 534 | 25.744 | 42.191 | 11.076 | 1.00 | 56.50 |
| ATOM | 3739 | CD2 | LEU | 534 | 25.605 | 40.098 | 9.718 | 1.00 | 57.05 |
| ATOM | 3740 | C | LEU | 534 | 24.041 | 43.928 | 8.220 | 1.00 | 60.52 |
| ATOM | 3741 | O | LEU | 534 | 25.201 | 44.337 | 8.284 | 1.00 | 60.29 |
| ATOM | 3742 | N | ALA | 535 | 23.303 | 44.010 | 7.118 | 1.00 | 62.13 |
| ATOM | 3743 | CA | ALA | 535 | 23.816 | 44.611 | 5.892 | 1.00 | 64.07 |
| ATOM | 3744 | CB | ALA | 535 | 22.930 | 44.228 | 4.713 | 1.00 | 64.06 |
| ATOM | 3745 | C | ALA | 535 | 23.859 | 46.127 | 6.048 | 1.00 | 65.26 |
| ATOM | 3746 | O | ALA | 535 | 24.618 | 46.817 | 5.363 | 1.00 | 65.11 |
| ATOM | 3747 | N | GLU | 536 | 23.034 | 46.633 | 6.959 | 1.00 | 66.37 |
| ATOM | 3748 | CA | GLU | 536 | 22.955 | 48.064 | 7.233 | 1.00 | 67.19 |
| ATOM | 3749 | CB | GLU | 536 | 21.583 | 48.408 | 7.823 | 1.00 | 68.14 |
| ATOM | 3750 | CG | GLU | 536 | 20.397 | 47.779 | 7.094 | 1.00 | 69.56 |
| ATOM | 3751 | CD | GLU | 536 | 20.159 | 48.366 | 5.713 | 1.00 | 70.40 |
| ATOM | 3752 | OE1 | GLU | 536 | 21.070 | 48.290 | 4.861 | 1.00 | 71.10 |
| ATOM | 3753 | OE2 | GLU | 536 | 19.053 | 48.904 | 5.481 | 1.00 | 70.27 |
| ATOM | 3754 | C | GLU | 536 | 24.050 | 48.445 | 8.228 | 1.00 | 66.96 |
| ATOM | 3755 | O | GLU | 536 | 24.057 | 49.552 | 8.765 | 1.00 | 67.14 |
| ATOM | 3756 | N | THR | 537 | 24.969 | 47.515 | 8.472 | 1.00 | 66.36 |
| ATOM | 3757 | CA | THR | 537 | 26.069 | 47.741 | 9.402 | 1.00 | 65.80 |
| ATOM | 3758 | CB | THR | 537 | 25.557 | 47.783 | 10.860 | 1.00 | 65.76 |
| ATOM | 3759 | OG1 | THR | 537 | 26.640 | 48.104 | 11.742 | 1.00 | 65.38 |
| ATOM | 3760 | CG2 | THR | 537 | 24.962 | 46.436 | 11.256 | 1.00 | 65.93 |
| ATOM | 3761 | C | THR | 537 | 27.121 | 46.637 | 9.269 | 1.00 | 65.16 |
| ATOM | 3762 | O | THR | 537 | 27.200 | 45.962 | 8.242 | 1.00 | 65.26 |
| ATOM | 3763 | N | GLY | 538 | 27.931 | 46.463 | 10.308 | 1.00 | 64.34 |
| ATOM | 3764 | CA | GLY | 538 | 28.959 | 45.439 | 10.282 | 1.00 | 63.04 |
| ATOM | 3765 | C | GLY | 538 | 29.062 | 44.734 | 11.617 | 1.00 | 62.37 |
| ATOM | 3766 | O | GLY | 538 | 30.023 | 44.011 | 11.883 | 1.00 | 62.04 |
| ATOM | 3767 | N | ASP | 539 | 28.060 | 44.949 | 12.462 | 1.00 | 61.49 |
| ATOM | 3768 | CA | ASP | 539 | 28.021 | 44.342 | 13.785 | 1.00 | 60.74 |
| ATOM | 3769 | CB | ASP | 539 | 28.268 | 45.407 | 14.857 | 1.00 | 62.06 |
| ATOM | 3770 | CG | ASP | 539 | 28.250 | 44.838 | 16.262 | 1.00 | 63.43 |
| ATOM | 3771 | OD1 | ASP | 539 | 28.222 | 45.633 | 17.226 | 1.00 | 63.49 |
| ATOM | 3772 | OD2 | ASP | 539 | 28.270 | 43.595 | 16.402 | 1.00 | 64.82 |
| ATOM | 3773 | C | ASP | 539 | 26.666 | 43.684 | 14.023 | 1.00 | 59.48 |
| ATOM | 3774 | O | ASP | 539 | 25.624 | 44.258 | 13.705 | 1.00 | 58.85 |
| ATOM | 3775 | N | ILE | 540 | 26.685 | 42.478 | 14.582 | 1.00 | 58.12 |
| ATOM | 3776 | CA | ILE | 540 | 25.455 | 41.747 | 14.867 | 1.00 | 56.14 |
| ATOM | 3777 | CB | ILE | 540 | 25.760 | 40.355 | 15.456 | 1.00 | 57.05 |
| ATOM | 3778 | CG2 | ILE | 540 | 24.458 | 39.622 | 15.772 | 1.00 | 56.67 |
| ATOM | 3779 | CG1 | ILE | 540 | 26.596 | 39.549 | 14.461 | 1.00 | 56.82 |
| ATOM | 3780 | CD1 | ILE | 540 | 27.153 | 38.264 | 15.030 | 1.00 | 57.97 |
| ATOM | 3781 | C | ILE | 540 | 24.612 | 42.534 | 15.864 | 1.00 | 54.58 |
| ATOM | 3782 | O | ILE | 540 | 23.410 | 42.714 | 15.669 | 1.00 | 53.94 |
| ATOM | 3783 | N | ARG | 541 | 25.252 | 43.002 | 16.932 | 1.00 | 52.23 |
| ATOM | 3784 | CA | ARG | 541 | 24.564 | 43.779 | 17.955 | 1.00 | 50.98 |
| ATOM | 3785 | CB | ARG | 541 | 25.524 | 44.108 | 19.100 | 1.00 | 49.65 |
| ATOM | 3786 | CG | ARG | 541 | 26.008 | 42.882 | 19.852 | 1.00 | 48.84 |
| ATOM | 3787 | CD | ARG | 541 | 27.008 | 43.238 | 20.938 | 1.00 | 47.15 |
| ATOM | 3788 | NE | ARG | 541 | 27.476 | 42.053 | 21.655 | 1.00 | 47.04 |
| ATOM | 3789 | CZ | ARG | 541 | 28.122 | 41.038 | 21.087 | 1.00 | 47.57 |
| ATOM | 3790 | NH1 | ARG | 541 | 28.383 | 41.054 | 19.787 | 1.00 | 47.71 |
| ATOM | 3791 | NH2 | ARG | 541 | 28.509 | 40.004 | 21.821 | 1.00 | 47.56 |
| ATOM | 3792 | C | ARG | 541 | 23.999 | 45.066 | 17.359 | 1.00 | 50.80 |
| ATOM | 3793 | O | ARG | 541 | 22.915 | 45.515 | 17.739 | 1.00 | 50.76 |
| ATOM | 3794 | N | ALA | 542 | 24.735 | 45.654 | 16.422 | 1.00 | 50.14 |
| ATOM | 3795 | CA | ALA | 542 | 24.297 | 46.882 | 15.770 | 1.00 | 49.97 |
| ATOM | 3796 | CB | ALA | 542 | 25.369 | 47.376 | 14.806 | 1.00 | 49.83 |
| ATOM | 3797 | C | ALA | 542 | 22.996 | 46.614 | 15.020 | 1.00 | 49.62 |
| ATOM | 3798 | O | ALA | 542 | 22.065 | 47.417 | 15.064 | 1.00 | 49.97 |
| ATOM | 3799 | N | ALA | 543 | 22.942 | 45.479 | 14.332 | 1.00 | 49.14 |
| ATOM | 3800 | CA | ALA | 543 | 21.753 | 45.095 | 13.583 | 1.00 | 48.56 |
| ATOM | 3801 | CB | ALA | 543 | 22.022 | 43.819 | 12.794 | 1.00 | 47.62 |
| ATOM | 3802 | C | ALA | 543 | 20.593 | 44.882 | 14.554 | 1.00 | 48.49 |
| ATOM | 3803 | O | ALA | 543 | 19.431 | 45.095 | 14.205 | 1.00 | 48.03 |
| ATOM | 3804 | N | VAL | 544 | 20.923 | 44.462 | 15.773 | 1.00 | 47.91 |
| ATOM | 3805 | CA | VAL | 544 | 19.921 | 44.220 | 16.807 | 1.00 | 48.15 |
| ATOM | 3806 | CB | VAL | 544 | 20.547 | 43.544 | 18.054 | 1.00 | 47.72 |
| ATOM | 3807 | CG1 | VAL | 544 | 19.493 | 43.354 | 19.133 | 1.00 | 48.12 |
| ATOM | 3808 | CG2 | VAL | 544 | 21.151 | 42.203 | 17.670 | 1.00 | 47.67 |
| ATOM | 3809 | C | VAL | 544 | 19.280 | 45.535 | 17.234 | 1.00 | 48.25 |
| ATOM | 3810 | O | VAL | 544 | 18.055 | 45.670 | 17.229 | 1.00 | 48.02 |
| ATOM | 3811 | N | ARG | 545 | 20.113 | 46.503 | 17.602 | 1.00 | 47.95 |
| ATOM | 3812 | CA | ARG | 545 | 19.613 | 47.804 | 18.025 | 1.00 | 48.51 |
| ATOM | 3813 | CB | ARG | 545 | 20.771 | 48.699 | 18.471 | 1.00 | 47.82 |
| ATOM | 3814 | CG | ARG | 545 | 21.478 | 48.204 | 19.722 | 1.00 | 49.07 |
| ATOM | 3815 | CD | ARG | 545 | 22.351 | 49.289 | 20.335 | 1.00 | 49.84 |
| ATOM | 3816 | NE | ARG | 545 | 23.473 | 49.659 | 19.477 | 1.00 | 51.11 |
| ATOM | 3817 | CZ | ARG | 545 | 24.521 | 48.877 | 19.235 | 1.00 | 51.17 |
| ATOM | 3818 | NH1 | ARG | 545 | 24.596 | 47.674 | 19.787 | 1.00 | 52.25 |
| ATOM | 3819 | NH2 | ARG | 545 | 25.497 | 49.300 | 18.444 | 1.00 | 51.42 |
| ATOM | 3820 | C | ARG | 545 | 18.826 | 48.481 | 16.908 | 1.00 | 48.39 |
| ATOM | 3821 | O | ARG | 545 | 17.806 | 49.119 | 17.161 | 1.00 | 47.66 |
| ATOM | 3822 | N | GLN | 546 | 19.296 | 48.338 | 15.674 | 1.00 | 49.12 |
| ATOM | 3823 | CA | GLN | 546 | 18.610 | 48.941 | 14.538 | 1.00 | 50.17 |
| ATOM | 3824 | CB | GLN | 546 | 19.428 | 48.766 | 13.257 | 1.00 | 51.51 |
| ATOM | 3825 | CG | GLN | 546 | 18.835 | 49.481 | 12.052 | 1.00 | 54.04 |
| ATOM | 3826 | CD | GLN | 546 | 19.635 | 49.256 | 10.786 | 1.00 | 55.80 |
| ATOM | 3827 | OE1 | GLN | 546 | 20.829 | 49.555 | 10.731 | 1.00 | 57.97 |
| ATOM | 3828 | NE2 | GLN | 546 | 18.980 | 48.730 | 9.757 | 1.00 | 56.73 |
| ATOM | 3829 | C | GLN | 546 | 17.245 | 48.282 | 14.369 | 1.00 | 50.05 |
| ATOM | 3830 | O | GLN | 546 | 16.268 | 48.936 | 14.001 | 1.00 | 49.98 |
| ATOM | 3831 | N | TYR | 547 | 17.189 | 46.982 | 14.640 | 1.00 | 49.32 |
| ATOM | 3832 | CA | TYR | 547 | 15.948 | 46.227 | 14.537 | 1.00 | 48.54 |
| ATOM | 3833 | CB | TYR | 547 | 16.203 | 44.750 | 14.840 | 1.00 | 49.78 |
| ATOM | 3834 | CG | TYR | 547 | 14.944 | 43.925 | 14.955 | 1.00 | 50.44 |
| ATOM | 3835 | CD1 | TYR | 547 | 14.056 | 43.822 | 13.885 | 1.00 | 51.69 |
| ATOM | 3836 | CE1 | TYR | 547 | 12.893 | 43.064 | 13.984 | 1.00 | 51.91 |
| ATOM | 3837 | CD2 | TYR | 547 | 14.637 | 43.249 | 16.135 | 1.00 | 51.50 |
| ATOM | 3838 | CE2 | TYR | 547 | 13.476 | 42.487 | 16.247 | 1.00 | 51.56 |
| ATOM | 3839 | CZ | TYR | 547 | 12.610 | 42.399 | 15.168 | 1.00 | 52.16 |
| ATOM | 3840 | OH | TYR | 547 | 11.463 | 41.646 | 15.271 | 1.00 | 51.53 |
| ATOM | 3841 | C | TYR | 547 | 14.926 | 46.781 | 15.523 | 1.00 | 47.25 |
| ATOM | 3842 | O | TYR | 547 | 13.793 | 47.092 | 15.155 | 1.00 | 46.01 |
| ATOM | 3843 | N | MET | 548 | 15.345 | 46.902 | 16.779 | 1.00 | 45.68 |
| ATOM | 3844 | CA | MET | 548 | 14.491 | 47.421 | 17.839 | 1.00 | 45.05 |
| ATOM | 3845 | CB | MET | 548 | 15.273 | 47.471 | 19.153 | 1.00 | 43.72 |
| ATOM | 3846 | CG | MET | 548 | 15.852 | 46.136 | 19.574 | 1.00 | 44.22 |
| ATOM | 3847 | SD | MET | 548 | 17.010 | 46.272 | 20.949 | 1.00 | 43.86 |
| ATOM | 3848 | CE | MET | 548 | 15.902 | 46.115 | 22.333 | 1.00 | 45.50 |
| ATOM | 3849 | C | MET | 548 | 14.016 | 48.823 | 17.479 | 1.00 | 45.08 |
| ATOM | 3850 | O | MET | 548 | 12.859 | 49.186 | 17.708 | 1.00 | 43.72 |
| ATOM | 3851 | N | ALA | 549 | 14.929 | 49.603 | 16.910 | 1.00 | 45.01 |
| ATOM | 3852 | CA | ALA | 549 | 14.643 | 50.973 | 16.515 | 1.00 | 45.18 |
| ATOM | 3853 | CB | ALA | 549 | 15.923 | 51.651 | 16.044 | 1.00 | 44.23 |
| ATOM | 3854 | C | ALA | 549 | 13.581 | 51.042 | 15.424 | 1.00 | 45.78 |
| ATOM | 3855 | O | ALA | 549 | 12.518 | 51.625 | 15.626 | 1.00 | 45.86 |
| ATOM | 3856 | N | GLU | 550 | 13.867 | 50.442 | 14.272 | 1.00 | 46.83 |
| ATOM | 3857 | CA | GLU | 550 | 12.925 | 50.460 | 13.156 | 1.00 | 48.63 |
| ATOM | 3858 | CB | GLU | 550 | 13.470 | 49.657 | 11.975 | 1.00 | 49.72 |
| ATOM | 3859 | CG | GLU | 550 | 14.515 | 50.394 | 11.160 | 1.00 | 51.94 |
| ATOM | 3860 | CD | GLU | 550 | 14.779 | 49.726 | 9.828 | 1.00 | 53.48 |
| ATOM | 3861 | OE1 | GLU | 550 | 13.799 | 49.459 | 9.097 | 1.00 | 54.23 |
| ATOM | 3862 | OE2 | GLU | 550 | 15.961 | 49.474 | 9.506 | 1.00 | 55.31 |
| ATOM | 3863 | C | GLU | 550 | 11.535 | 49.946 | 13.514 | 1.00 | 49.36 |
| ATOM | 3864 | O | GLU | 550 | 10.530 | 50.507 | 13.073 | 1.00 | 49.22 |
| ATOM | 3865 | N | VAL | 551 | 11.477 | 48.879 | 14.303 | 1.00 | 48.98 |
| ATOM | 3866 | CA | VAL | 551 | 10.199 | 48.310 | 14.707 | 1.00 | 49.91 |
| ATOM | 3867 | CB | VAL | 551 | 10.397 | 47.020 | 15.538 | 1.00 | 49.52 |
| ATOM | 3868 | CG1 | VAL | 551 | 9.066 | 46.544 | 16.094 | 1.00 | 49.11 |
| ATOM | 3869 | CG2 | VAL | 551 | 11.017 | 45.937 | 14.668 | 1.00 | 49.69 |
| ATOM | 3870 | C | VAL | 551 | 9.409 | 49.318 | 15.536 | 1.00 | 50.71 |
| ATOM | 3871 | O | VAL | 551 | 8.193 | 49.444 | 15.385 | 1.00 | 50.53 |
| ATOM | 3872 | N | GLU | 552 | 10.110 | 50.036 | 16.408 | 1.00 | 51.60 |
| ATOM | 3873 | CA | GLU | 552 | 9.481 | 51.032 | 17.264 | 1.00 | 52.58 |
| ATOM | 3874 | CB | GLU | 552 | 10.379 | 51.324 | 18.468 | 1.00 | 53.60 |
| ATOM | 3875 | CG | GLU | 552 | 9.753 | 52.241 | 19.503 | 1.00 | 55.39 |
| ATOM | 3876 | CD | GLU | 552 | 10.650 | 52.453 | 20.704 | 1.00 | 56.32 |
| ATOM | 3877 | OE1 | GLU | 552 | 10.199 | 53.087 | 21.682 | 1.00 | 56.45 |
| ATOM | 3878 | OE2 | GLU | 552 | 11.808 | 51.987 | 20.670 | 1.00 | 57.66 |
| ATOM | 3879 | C | GLU | 552 | 9.217 | 52.323 | 16.492 | 1.00 | 53.07 |
| ATOM | 3880 | O | GLU | 552 | 8.295 | 53.073 | 16.811 | 1.00 | 52.73 |
| ATOM | 3881 | N | SER | 553 | 10.035 | 52.570 | 15.474 | 1.00 | 52.74 |
| ATOM | 3882 | CA | SER | 553 | 9.903 | 53.762 | 14.651 | 1.00 | 53.94 |
| ATOM | 3883 | CB | SER | 553 | 11.230 | 54.065 | 13.956 | 1.00 | 54.05 |
| ATOM | 3884 | OG | SER | 553 | 12.264 | 54.253 | 14.908 | 1.00 | 56.04 |
| ATOM | 3885 | C | SER | 553 | 8.811 | 53.583 | 13.603 | 1.00 | 54.10 |
| ATOM | 3886 | O | SER | 553 | 8.314 | 54.556 | 13.034 | 1.00 | 53.78 |
| ATOM | 3887 | N | GLY | 554 | 8.439 | 52.333 | 13.356 | 1.00 | 53.73 |
| ATOM | 3888 | CA | GLY | 554 | 7.413 | 52.052 | 12.371 | 1.00 | 53.14 |
| ATOM | 3889 | C | GLY | 554 | 8.037 | 51.889 | 11.003 | 1.00 | 53.03 |
| ATOM | 3890 | O | GLY | 554 | 7.353 | 51.571 | 10.030 | 1.00 | 52.59 |
| ATOM | 3891 | N | VAL | 555 | 9.347 | 52.109 | 10.931 | 1.00 | 52.96 |
| ATOM | 3892 | CA | VAL | 555 | 10.079 | 51.986 | 9.677 | 1.00 | 52.87 |
| ATOM | 3893 | CB | VAL | 555 | 11.565 | 52.367 | 9.862 | 1.00 | 53.58 |
| ATOM | 3894 | CG1 | VAL | 555 | 12.285 | 52.329 | 8.523 | 1.00 | 52.84 |
| ATOM | 3895 | CG2 | VAL | 555 | 11.671 | 53.749 | 10.485 | 1.00 | 53.08 |
| ATOM | 3896 | C | VAL | 555 | 9.998 | 50.554 | 9.156 | 1.00 | 52.67 |
| ATOM | 3897 | O | VAL | 555 | 9.908 | 50.324 | 7.948 | 1.00 | 52.23 |
| ATOM | 3898 | N | TYR | 556 | 10.031 | 49.595 | 10.076 | 1.00 | 52.50 |
| ATOM | 3899 | CA | TYR | 556 | 9.960 | 48.183 | 9.719 | 1.00 | 51.70 |
| ATOM | 3900 | CB | TYR | 556 | 11.227 | 47.456 | 10.174 | 1.00 | 51.86 |
| ATOM | 3901 | CG | TYR | 556 | 11.214 | 45.977 | 9.868 | 1.00 | 51.09 |
| ATOM | 3902 | CD1 | TYR | 556 | 11.254 | 45.519 | 8.552 | 1.00 | 50.94 |
| ATOM | 3903 | CE1 | TYR | 556 | 11.189 | 44.160 | 8.260 | 1.00 | 51.28 |
| ATOM | 3904 | CD2 | TYR | 556 | 11.113 | 45.034 | 10.892 | 1.00 | 51.14 |
| ATOM | 3905 | CE2 | TYR | 556 | 11.047 | 43.670 | 10.613 | 1.00 | 50.47 |
| ATOM | 3906 | CZ | TYR | 556 | 11.083 | 43.241 | 9.295 | 1.00 | 50.81 |
| ATOM | 3907 | OH | TYR | 556 | 10.995 | 41.900 | 9.000 | 1.00 | 50.30 |
| ATOM | 3908 | C | TYR | 556 | 8.741 | 47.531 | 10.363 | 1.00 | 51.68 |
| ATOM | 3909 | O | TYR | 556 | 8.529 | 47.653 | 11.569 | 1.00 | 51.15 |
| ATOM | 3910 | N | PRO | 557 | 7.927 | 46.820 | 9.565 | 1.00 | 52.14 |
| ATOM | 3911 | CD | PRO | 557 | 6.797 | 46.019 | 10.067 | 1.00 | 52.66 |
| ATOM | 3912 | CA | PRO | 557 | 8.100 | 46.621 | 8.120 | 1.00 | 52.90 |
| ATOM | 3913 | CB | PRO | 557 | 7.223 | 45.405 | 7.840 | 1.00 | 52.64 |
| ATOM | 3914 | CG | PRO | 557 | 6.089 | 45.614 | 8.786 | 1.00 | 52.85 |
| ATOM | 3915 | C | PRO | 557 | 7.684 | 47.839 | 7.292 | 1.00 | 53.09 |
| ATOM | 3916 | O | PRO | 557 | 6.762 | 48.566 | 7.664 | 1.00 | 53.40 |
| ATOM | 3917 | N | GLY | 558 | 8.366 | 48.050 | 6.170 | 1.00 | 53.27 |
| ATOM | 3918 | CA | GLY | 558 | 8.055 | 49.181 | 5.316 | 1.00 | 53.66 |
| ATOM | 3919 | C | GLY | 558 | 6.840 | 48.937 | 4.444 | 1.00 | 54.13 |
| ATOM | 3920 | O | GLY | 558 | 5.703 | 49.072 | 4.900 | 1.00 | 54.10 |
| ATOM | 3921 | N | GLU | 559 | 7.081 | 48.580 | 3.186 | 1.00 | 53.86 |
| ATOM | 3922 | CA | GLU | 559 | 6.001 | 48.309 | 2.245 | 1.00 | 54.22 |
| ATOM | 3923 | CB | GLU | 559 | 5.822 | 49.482 | 1.276 | 1.00 | 55.40 |
| ATOM | 3924 | CG | GLU | 559 | 4.536 | 49.415 | 0.461 | 1.00 | 56.21 |
| ATOM | 3925 | CD | GLU | 559 | 3.295 | 49.590 | 1.321 | 1.00 | 56.50 |
| ATOM | 3926 | OE1 | GLU | 559 | 2.175 | 49.406 | 0.800 | 1.00 | 57.07 |
| ATOM | 3927 | OE2 | GLU | 559 | 3.440 | 49.916 | 2.518 | 1.00 | 57.08 |
| ATOM | 3928 | C | GLU | 559 | 6.295 | 47.039 | 1.452 | 1.00 | 54.13 |
| ATOM | 3929 | O | GLU | 559 | 5.378 | 46.336 | 1.026 | 1.00 | 53.98 |
| ATOM | 3930 | N | GLU | 560 | 7.576 | 46.751 | 1.253 | 1.00 | 53.68 |
| ATOM | 3931 | CA | GLU | 560 | 7.977 | 45.560 | 0.515 | 1.00 | 54.17 |
| ATOM | 3932 | CB | GLU | 560 | 9.400 | 45.719 | −0.027 | 1.00 | 54.49 |
| ATOM | 3933 | CG | GLU | 560 | 9.861 | 47.161 | −0.132 | 1.00 | 55.28 |
| ATOM | 3934 | CD | GLU | 560 | 10.388 | 47.700 | 1.186 | 1.00 | 55.88 |
| ATOM | 3935 | OE1 | GLU | 560 | 11.523 | 47.335 | 1.563 | 1.00 | 55.57 |
| ATOM | 3936 | OE2 | GLU | 560 | 9.668 | 48.478 | 1.850 | 1.00 | 55.73 |
| ATOM | 3937 | C | GLU | 560 | 7.912 | 44.352 | 1.441 | 1.00 | 53.99 |
| ATOM | 3938 | O | GLU | 560 | 8.119 | 43.215 | 1.014 | 1.00 | 54.19 |
| ATOM | 3939 | N | HIS | 561 | 7.621 | 44.616 | 2.712 | 1.00 | 53.66 |
| ATOM | 3940 | CA | HIS | 561 | 7.527 | 43.571 | 3.727 | 1.00 | 53.03 |
| ATOM | 3941 | CB | HIS | 561 | 8.320 | 43.975 | 4.972 | 1.00 | 53.33 |
| ATOM | 3942 | CG | HIS | 561 | 9.687 | 44.507 | 4.676 | 1.00 | 52.71 |
| ATOM | 3943 | CD2 | HIS | 561 | 10.242 | 45.720 | 4.914 | 1.00 | 52.67 |
| ATOM | 3944 | ND1 | HIS | 561 | 10.662 | 43.755 | 4.057 | 1.00 | 52.82 |
| ATOM | 3945 | CE1 | HIS | 561 | 11.758 | 44.482 | 3.926 | 1.00 | 53.52 |
| ATOM | 3946 | NE2 | HIS | 561 | 11.529 | 45.677 | 4.439 | 1.00 | 52.58 |
| ATOM | 3947 | C | HIS | 561 | 6.073 | 43.340 | 4.124 | 1.00 | 52.40 |
| ATOM | 3948 | O | HIS | 561 | 5.783 | 42.508 | 4.983 | 1.00 | 52.28 |
| ATOM | 3949 | N | SER | 562 | 5.164 | 44.081 | 3.495 | 1.00 | 51.41 |
| ATOM | 3950 | CA | SER | 562 | 3.741 | 43.973 | 3.794 | 1.00 | 50.51 |
| ATOM | 3951 | CB | SER | 562 | 3.172 | 45.359 | 4.106 | 1.00 | 50.14 |
| ATOM | 3952 | OG | SER | 562 | 3.952 | 46.021 | 5.086 | 1.00 | 49.54 |
| ATOM | 3953 | C | SER | 562 | 2.956 | 43.350 | 2.644 | 1.00 | 50.99 |
| ATOM | 3954 | O | SER | 562 | 3.381 | 43.398 | 1.488 | 1.00 | 50.00 |
| ATOM | 3955 | N | PHE | 563 | 1.808 | 42.764 | 2.966 | 1.00 | 50.96 |
| ATOM | 3956 | CA | PHE | 563 | 0.966 | 42.144 | 1.953 | 1.00 | 51.90 |
| ATOM | 3957 | CB | PHE | 563 | 0.792 | 40.649 | 2.230 | 1.00 | 51.88 |
| ATOM | 3958 | CG | PHE | 563 | 2.085 | 39.889 | 2.291 | 1.00 | 52.62 |
| ATOM | 3959 | CD1 | PHE | 563 | 2.800 | 39.800 | 3.482 | 1.00 | 52.62 |
| ATOM | 3960 | CD2 | PHE | 563 | 2.598 | 39.274 | 1.154 | 1.00 | 52.42 |
| ATOM | 3961 | CE1 | PHE | 563 | 4.008 | 39.107 | 3.540 | 1.00 | 53.30 |
| ATOM | 3962 | CE2 | PHE | 563 | 3.805 | 38.580 | 1.200 | 1.00 | 53.30 |
| ATOM | 3963 | CZ | PHE | 563 | 4.512 | 38.496 | 2.397 | 1.00 | 53.09 |
| ATOM | 3964 | C | PHE | 563 | −0.396 | 42.820 | 1.925 | 1.00 | 52.18 |
| ATOM | 3965 | O | PHE | 563 | −0.847 | 43.371 | 2.930 | 1.00 | 51.96 |
| ATOM | 3966 | N | HIS | 564 | −1.051 | 42.775 | 0.771 | 1.00 | 53.34 |
| ATOM | 3967 | CA | HIS | 564 | −2.362 | 43.394 | 0.617 | 1.00 | 54.06 |
| ATOM | 3968 | CB | HIS | 564 | −2.228 | 44.709 | −0.155 | 1.00 | 54.83 |
| ATOM | 3969 | CG | HIS | 564 | −1.305 | 45.696 | 0.493 | 1.00 | 55.50 |
| ATOM | 3970 | CD2 | HIS | 564 | −0.118 | 46.206 | 0.086 | 1.00 | 55.30 |
| ATOM | 3971 | ND1 | HIS | 564 | −1.564 | 46.262 | 1.722 | 1.00 | 55.51 |
| ATOM | 3972 | CE1 | HIS | 564 | −0.577 | 47.079 | 2.046 | 1.00 | 55.52 |
| ATOM | 3973 | NE2 | HIS | 564 | 0.314 | 47.062 | 1.070 | 1.00 | 55.47 |
| ATOM | 3974 | C | HIS | 564 | −3.315 | 42.457 | −0.111 | 1.00 | 54.29 |
| ATOM | 3975 | O | HIS | 564 | −4.385 | 42.152 | 0.454 | 1.00 | 54.03 |
| ATOM | 3976 | OXT | HIS | 564 | −2.978 | 42.040 | −1.238 | 1.00 | 55.99 |
| ATOM | 3977 | C1 | KPL | 565 | 8.381 | 32.905 | 12.296 | 1.00 | 41.48 |
| ATOM | 3978 | C2 | KPL | 565 | 7.795 | 33.008 | 13.719 | 1.00 | 41.70 |
| ATOM | 3979 | C3 | KPL | 565 | 8.747 | 33.842 | 14.588 | 1.00 | 41.04 |
| ATOM | 3980 | C4 | KPL | 565 | 6.432 | 33.729 | 13.657 | 1.00 | 42.99 |
| ATOM | 3981 | O1 | KPL | 565 | 5.502 | 32.994 | 12.852 | 1.00 | 47.23 |
| ATOM | 3982 | C5 | KPL | 565 | 7.641 | 31.587 | 14.333 | 1.00 | 41.12 |
| ATOM | 3983 | O2 | KPL | 565 | 6.548 | 31.189 | 14.686 | 1.00 | 40.23 |
| ATOM | 3984 | C6 | KPL | 565 | 8.827 | 30.664 | 14.510 | 1.00 | 39.79 |
| ATOM | 3985 | O3 | KPL | 565 | 9.940 | 31.012 | 14.179 | 1.00 | 41.42 |
| ATOM | 3986 | O4 | KPL | 565 | 8.649 | 29.440 | 15.040 | 1.00 | 37.97 |
| ATOM | 3987 | CB | MET | 601 | 10.230 | 21.149 | −10.646 | 1.00 | 69.60 |
| ATOM | 3988 | CG | MET | 601 | 9.281 | 20.343 | −11.531 | 1.00 | 71.50 |
| ATOM | 3989 | SD | MET | 601 | 7.632 | 21.063 | −11.738 | 1.00 | 73.80 |
| ATOM | 3990 | CE | MET | 601 | 6.646 | 19.961 | −10.716 | 1.00 | 73.00 |
| ATOM | 3991 | C | MET | 601 | 8.842 | 22.547 | −9.089 | 1.00 | 66.93 |
| ATOM | 3992 | O | MET | 601 | 8.861 | 23.439 | −9.940 | 1.00 | 67.03 |
| ATOM | 3993 | N | MET | 601 | 9.123 | 20.086 | −8.698 | 1.00 | 68.90 |
| ATOM | 3994 | CA | MET | 601 | 9.767 | 21.337 | −9.196 | 1.00 | 68.18 |
| ATOM | 3995 | N | LYS | 602 | 8.036 | 22.570 | −8.032 | 1.00 | 64.76 |
| ATOM | 3996 | CA | LYS | 602 | 7.110 | 23.667 | −7.796 | 1.00 | 62.08 |
| ATOM | 3997 | CB | LYS | 602 | 5.693 | 23.272 | −8.237 | 1.00 | 63.38 |
| ATOM | 3998 | CG | LYS | 602 | 5.557 | 23.006 | −9.732 | 1.00 | 64.55 |
| ATOM | 3999 | CD | LYS | 602 | 5.683 | 24.288 | −10.545 | 1.00 | 65.81 |
| ATOM | 4000 | CE | LYS | 602 | 4.492 | 25.207 | −10.311 | 1.00 | 65.96 |
| ATOM | 4001 | NZ | LYS | 602 | 4.629 | 26.497 | −11.038 | 1.00 | 66.71 |
| ATOM | 4002 | C | LYS | 602 | 7.105 | 24.081 | −6.322 | 1.00 | 59.15 |
| ATOM | 4003 | O | LYS | 602 | 7.250 | 25.263 | −6.006 | 1.00 | 59.60 |
| ATOM | 4004 | N | PRO | 603 | 6.959 | 23.112 | −5.396 | 1.00 | 55.04 |
| ATOM | 4005 | CD | PRO | 603 | 7.155 | 23.394 | −3.964 | 1.00 | 54.14 |
| ATOM | 4006 | CA | PRO | 603 | 6.772 | 21.673 | −5.613 | 1.00 | 51.11 |
| ATOM | 4007 | CB | PRO | 603 | 7.269 | 21.076 | −4.303 | 1.00 | 52.30 |
| ATOM | 4008 | CG | PRO | 603 | 6.782 | 22.075 | −3.311 | 1.00 | 53.08 |
| ATOM | 4009 | C | PRO | 603 | 5.317 | 21.318 | −5.889 | 1.00 | 46.82 |
| ATOM | 4010 | O | PRO | 603 | 4.426 | 22.143 | −5.704 | 1.00 | 47.26 |
| ATOM | 4011 | N | THR | 604 | 5.079 | 20.088 | −6.329 | 1.00 | 41.84 |
| ATOM | 4012 | CA | THR | 604 | 3.727 | 19.639 | −6.627 | 1.00 | 36.41 |
| ATOM | 4013 | CB | THR | 604 | 3.742 | 18.241 | −7.268 | 1.00 | 36.28 |
| ATOM | 4014 | OG1 | THR | 604 | 4.564 | 18.265 | −8.440 | 1.00 | 34.78 |
| ATOM | 4015 | CG2 | THR | 604 | 2.331 | 17.811 | −7.650 | 1.00 | 35.28 |
| ATOM | 4016 | C | THR | 604 | 2.902 | 19.582 | −5.350 | 1.00 | 33.25 |
| ATOM | 4017 | O | THR | 604 | 3.270 | 18.893 | −4.398 | 1.00 | 31.12 |
| ATOM | 4018 | N | THR | 605 | 1.792 | 20.314 | −5.326 | 1.00 | 30.68 |
| ATOM | 4019 | CA | THR | 605 | 0.929 | 20.327 | −4.153 | 1.00 | 29.21 |
| ATOM | 4020 | CB | THR | 605 | 0.921 | 21.708 | −3.465 | 1.00 | 29.89 |
| ATOM | 4021 | OG1 | THR | 605 | 0.134 | 22.625 | −4.236 | 1.00 | 30.44 |
| ATOM | 4022 | CG2 | THR | 605 | 2.341 | 22.245 | −3.336 | 1.00 | 29.99 |
| ATOM | 4023 | C | THR | 605 | −0.505 | 19.970 | −4.524 | 1.00 | 27.07 |
| ATOM | 4024 | O | THR | 605 | −0.823 | 19.779 | −5.700 | 1.00 | 25.26 |
| ATOM | 4025 | N | ILE | 606 | −1.358 | 19.885 | −3.508 | 1.00 | 24.97 |
| ATOM | 4026 | CA | ILE | 606 | −2.767 | 19.551 | −3.684 | 1.00 | 24.26 |
| ATOM | 4027 | CB | ILE | 606 | −3.495 | 19.497 | −2.318 | 1.00 | 26.83 |
| ATOM | 4028 | CG2 | ILE | 606 | −4.942 | 19.052 | −2.510 | 1.00 | 24.52 |
| ATOM | 4029 | CG1 | ILE | 606 | −2.767 | 18.531 | −1.381 | 1.00 | 28.76 |
| ATOM | 4030 | CD1 | ILE | 606 | −3.204 | 18.634 | 0.077 | 1.00 | 32.70 |
| ATOM | 4031 | C | ILE | 606 | −3.459 | 20.593 | −4.558 | 1.00 | 22.72 |
| ATOM | 4032 | O | ILE | 606 | −4.297 | 20.259 | −5.397 | 1.00 | 21.59 |
| ATOM | 4033 | N | SER | 607 | −3.095 | 21.854 | −4.359 | 1.00 | 21.35 |
| ATOM | 4034 | CA | SER | 607 | −3.691 | 22.947 | −5.113 | 1.00 | 21.40 |
| ATOM | 4035 | CB | SER | 607 | −3.042 | 24.271 | −4.712 | 1.00 | 23.61 |
| ATOM | 4036 | OG | SER | 607 | −3.247 | 24.522 | −3.332 | 1.00 | 30.17 |
| ATOM | 4037 | C | SER | 607 | −3.552 | 22.744 | −6.618 | 1.00 | 20.50 |
| ATOM | 4038 | O | SER | 607 | −4.427 | 23.140 | −7.390 | 1.00 | 19.39 |
| ATOM | 4039 | N | LEU | 608 | −2.448 | 22.129 | −7.025 | 1.00 | 20.28 |
| ATOM | 4040 | CA | LEU | 608 | −2.194 | 21.879 | −8.437 | 1.00 | 20.79 |
| ATOM | 4041 | CB | LEU | 608 | −0.773 | 21.349 | −8.636 | 1.00 | 22.00 |
| ATOM | 4042 | CG | LEU | 608 | 0.040 | 21.923 | −9.802 | 1.00 | 25.44 |
| ATOM | 4043 | CD1 | LEU | 608 | 1.261 | 21.050 | −10.024 | 1.00 | 26.12 |
| ATOM | 4044 | CD2 | LEU | 608 | −0.794 | 21.996 | −11.055 | 1.00 | 26.06 |
| ATOM | 4045 | C | LEU | 608 | −3.196 | 20.859 | −8.979 | 1.00 | 18.78 |
| ATOM | 4046 | O | LEU | 608 | −3.749 | 21.022 | −10.068 | 1.00 | 16.08 |
| ATOM | 4047 | N | LEU | 609 | −3.425 | 19.799 | −8.216 | 1.00 | 16.93 |
| ATOM | 4048 | CA | LEU | 609 | −4.355 | 18.770 | −8.645 | 1.00 | 15.90 |
| ATOM | 4049 | CB | LEU | 609 | −4.284 | 17.564 | −7.715 | 1.00 | 14.64 |
| ATOM | 4050 | CG | LEU | 609 | −2.919 | 16.894 | −7.626 | 1.00 | 16.05 |
| ATOM | 4051 | CD1 | LEU | 609 | −3.088 | 15.620 | −6.811 | 1.00 | 14.29 |
| ATOM | 4052 | CD2 | LEU | 609 | −2.367 | 16.582 | −9.012 | 1.00 | 14.89 |
| ATOM | 4053 | C | LEU | 609 | −5.776 | 19.302 | −8.684 | 1.00 | 15.23 |
| ATOM | 4054 | O | LEU | 609 | −6.549 | 18.943 | −9.564 | 1.00 | 16.80 |
| ATOM | 4055 | N | GLN | 610 | −6.120 | 20.157 | −7.727 | 1.00 | 16.25 |
| ATOM | 4056 | CA | GLN | 610 | −7.454 | 20.734 | −7.688 | 1.00 | 18.78 |
| ATOM | 4057 | CB | GLN | 610 | −7.610 | 21.601 | −6.431 | 1.00 | 22.10 |
| ATOM | 4058 | CG | GLN | 610 | −9.015 | 22.149 | −6.168 | 1.00 | 22.61 |
| ATOM | 4059 | CD | GLN | 610 | −10.087 | 21.070 | −6.066 | 1.00 | 25.18 |
| ATOM | 4060 | OE1 | GLN | 610 | −10.864 | 20.862 | −7.000 | 1.00 | 25.07 |
| ATOM | 4061 | NE2 | GLN | 610 | −10.135 | 20.380 | −4.930 | 1.00 | 23.46 |
| ATOM | 4062 | C | GLN | 610 | −7.606 | 21.564 | −8.962 | 1.00 | 19.36 |
| ATOM | 4063 | O | GLN | 610 | −8.674 | 21.599 | −9.567 | 1.00 | 20.25 |
| ATOM | 4064 | N | LYS | 611 | −6.518 | 22.203 | −9.380 | 1.00 | 17.95 |
| ATOM | 4065 | CA | LYS | 611 | −6.527 | 23.011 | −10.591 | 1.00 | 19.00 |
| ATOM | 4066 | CB | LYS | 611 | −5.237 | 23.829 | −10.694 | 1.00 | 19.60 |
| ATOM | 4067 | CG | LYS | 611 | −5.135 | 24.652 | −11.967 | 1.00 | 26.12 |
| ATOM | 4068 | CD | LYS | 611 | −3.929 | 25.572 | −11.947 | 1.00 | 30.05 |
| ATOM | 4069 | CE | LYS | 611 | −4.051 | 26.611 | −10.841 | 1.00 | 33.03 |
| ATOM | 4070 | NZ | LYS | 611 | −2.950 | 27.626 | −10.883 | 1.00 | 36.49 |
| ATOM | 4071 | C | LYS | 611 | −6.684 | 22.119 | −11.825 | 1.00 | 18.37 |
| ATOM | 4072 | O | LYS | 611 | −7.438 | 22.443 | −12.743 | 1.00 | 17.92 |
| ATOM | 4073 | N | TYR | 612 | −5.969 | 21.000 | −11.853 | 1.00 | 17.93 |
| ATOM | 4074 | CA | TYR | 612 | −6.069 | 20.072 | −12.974 | 1.00 | 17.14 |
| ATOM | 4075 | CB | TYR | 612 | −5.137 | 18.873 | −12.757 | 1.00 | 18.22 |
| ATOM | 4076 | CG | TYR | 612 | −3.674 | 19.182 | −13.000 | 1.00 | 20.39 |
| ATOM | 4077 | CD1 | TYR | 612 | −2.672 | 18.314 | −12.568 | 1.00 | 20.70 |
| ATOM | 4078 | CE1 | TYR | 612 | −1.325 | 18.591 | −12.819 | 1.00 | 22.32 |
| ATOM | 4079 | CD2 | TYR | 612 | −3.292 | 20.335 | −13.686 | 1.00 | 20.10 |
| ATOM | 4080 | CE2 | TYR | 612 | −1.952 | 20.618 | −13.941 | 1.00 | 22.45 |
| ATOM | 4081 | CZ | TYR | 612 | −0.978 | 19.745 | −13.507 | 1.00 | 20.89 |
| ATOM | 4082 | OH | TYR | 612 | 0.343 | 20.026 | −13.781 | 1.00 | 24.80 |
| ATOM | 4083 | C | TYR | 612 | −7.501 | 19.586 | −13.165 | 1.00 | 16.86 |
| ATOM | 4084 | O | TYR | 612 | −7.942 | 19.383 | −14.295 | 1.00 | 17.28 |
| ATOM | 4085 | N | LYS | 613 | −8.233 | 19.390 | −12.068 | 1.00 | 16.32 |
| ATOM | 4086 | CA | LYS | 613 | −9.618 | 18.935 | −12.192 | 1.00 | 15.88 |
| ATOM | 4087 | CB | LYS | 613 | −10.229 | 18.609 | −10.821 | 1.00 | 14.30 |
| ATOM | 4088 | CG | LYS | 613 | −11.689 | 18.175 | −10.928 | 1.00 | 17.16 |
| ATOM | 4089 | CD | LYS | 613 | −12.164 | 17.374 | −9.723 | 1.00 | 17.49 |
| ATOM | 4090 | CE | LYS | 613 | −13.620 | 16.936 | −9.921 | 1.00 | 18.61 |
| ATOM | 4091 | NZ | LYS | 613 | −14.048 | 15.860 | −8.988 | 1.00 | 17.77 |
| ATOM | 4092 | C | LYS | 613 | −10.422 | 20.045 | −12.869 | 1.00 | 16.11 |
| ATOM | 4093 | O | LYS | 613 | −11.313 | 19.786 | −13.676 | 1.00 | 16.71 |
| ATOM | 4094 | N | GLN | 614 | −10.087 | 21.285 | −12.523 | 1.00 | 17.86 |
| ATOM | 4095 | CA | GLN | 614 | −10.736 | 22.457 | −13.099 | 1.00 | 18.80 |
| ATOM | 4096 | CB | GLN | 614 | −10.097 | 23.740 | −12.558 | 1.00 | 21.10 |
| ATOM | 4097 | CG | GLN | 614 | −10.022 | 23.863 | −11.039 | 1.00 | 30.74 |
| ATOM | 4098 | CD | GLN | 614 | −9.094 | 24.991 | −10.574 | 1.00 | 35.26 |
| ATOM | 4099 | OE1 | GLN | 614 | −8.799 | 25.925 | −11.331 | 1.00 | 37.92 |
| ATOM | 4100 | NE2 | GLN | 614 | −8.645 | 24.916 | −9.315 | 1.00 | 33.37 |
| ATOM | 4101 | C | GLN | 614 | −10.534 | 22.416 | −14.613 | 1.00 | 17.17 |
| ATOM | 4102 | O | GLN | 614 | −11.455 | 22.671 | −15.390 | 1.00 | 18.11 |
| ATOM | 4103 | N | GLU | 615 | −9.314 | 22.085 | −15.016 | 1.00 | 16.94 |
| ATOM | 4104 | CA | GLU | 615 | −8.945 | 22.027 | −16.426 | 1.00 | 16.23 |
| ATOM | 4105 | CB | GLU | 615 | −7.451 | 22.331 | −16.557 | 1.00 | 16.41 |
| ATOM | 4106 | CG | GLU | 615 | −7.071 | 23.645 | −15.899 | 1.00 | 16.28 |
| ATOM | 4107 | CD | GLU | 615 | −5.579 | 23.851 | −15.792 | 1.00 | 16.90 |
| ATOM | 4108 | OE1 | GLU | 615 | −4.826 | 22.881 | −16.002 | 1.00 | 20.30 |
| ATOM | 4109 | OE2 | GLU | 615 | −5.150 | 24.981 | −15.478 | 1.00 | 17.87 |
| ATOM | 4110 | C | GLU | 615 | −9.277 | 20.707 | −17.115 | 1.00 | 16.72 |
| ATOM | 4111 | O | GLU | 615 | −9.025 | 20.543 | −18.310 | 1.00 | 16.29 |
| ATOM | 4112 | N | LYS | 616 | −9.869 | 19.778 | −16.371 | 1.00 | 17.52 |
| ATOM | 4113 | CA | LYS | 616 | −10.210 | 18.460 | −16.915 | 1.00 | 20.17 |
| ATOM | 4114 | CB | LYS | 616 | −11.254 | 18.567 | −18.027 | 1.00 | 23.07 |
| ATOM | 4115 | CG | LYS | 616 | −12.604 | 19.120 | −17.606 | 1.00 | 29.30 |
| ATOM | 4116 | CD | LYS | 616 | −13.410 | 18.131 | −16.789 | 1.00 | 32.57 |
| ATOM | 4117 | CE | LYS | 616 | −14.817 | 18.660 | −16.536 | 1.00 | 33.31 |
| ATOM | 4118 | NZ | LYS | 616 | −15.508 | 18.981 | −17.807 | 1.00 | 38.09 |
| ATOM | 4119 | C | LYS | 616 | −8.950 | 17.815 | −17.475 | 1.00 | 21.00 |
| ATOM | 4120 | O | LYS | 616 | −8.993 | 17.058 | −18.455 | 1.00 | 21.94 |
| ATOM | 4121 | N | LYS | 617 | −7.818 | 18.138 | −16.864 | 1.00 | 18.27 |
| ATOM | 4122 | CA | LYS | 617 | −6.557 | 17.569 | −17.298 | 1.00 | 17.56 |
| ATOM | 4123 | CB | LYS | 617 | −5.427 | 18.592 | −17.183 | 1.00 | 20.70 |
| ATOM | 4124 | CG | LYS | 617 | −4.111 | 18.075 | −17.736 | 1.00 | 23.47 |
| ATOM | 4125 | CD | LYS | 617 | −2.959 | 18.959 | −17.311 | 1.00 | 27.46 |
| ATOM | 4126 | CE | LYS | 617 | −3.104 | 20.376 | −17.847 | 1.00 | 28.51 |
| ATOM | 4127 | NZ | LYS | 617 | −2.146 | 21.290 | −17.160 | 1.00 | 31.20 |
| ATOM | 4128 | C | LYS | 617 | −6.266 | 16.375 | −16.399 | 1.00 | 16.64 |
| ATOM | 4129 | O | LYS | 617 | −5.821 | 16.535 | −15.263 | 1.00 | 14.57 |
| ATOM | 4130 | N | ARG | 618 | −6.536 | 15.180 | −16.910 | 1.00 | 16.67 |
| ATOM | 4131 | CA | ARG | 618 | −6.320 | 13.957 | −16.149 | 1.00 | 14.83 |
| ATOM | 4132 | CB | ARG | 618 | −6.910 | 12.773 | −16.921 | 1.00 | 15.62 |
| ATOM | 4133 | CG | ARG | 618 | −8.434 | 12.769 | −16.882 | 1.00 | 18.58 |
| ATOM | 4134 | CD | ARG | 618 | −9.058 | 11.769 | −17.852 | 1.00 | 23.21 |
| ATOM | 4135 | NE | ARG | 618 | −8.928 | 12.221 | −19.232 | 1.00 | 26.25 |
| ATOM | 4136 | CZ | ARG | 618 | −9.601 | 11.709 | −20.258 | 1.00 | 27.82 |
| ATOM | 4137 | NH1 | ARG | 618 | −10.458 | 10.716 | −20.060 | 1.00 | 27.55 |
| ATOM | 4138 | NH2 | ARG | 618 | −9.421 | 12.201 | −21.480 | 1.00 | 25.20 |
| ATOM | 4139 | C | ARG | 618 | −4.838 | 13.767 | −15.865 | 1.00 | 15.48 |
| ATOM | 4140 | O | ARG | 618 | −3.999 | 13.923 | −16.748 | 1.00 | 16.70 |
| ATOM | 4141 | N | PHE | 619 | −4.519 | 13.432 | −14.619 | 1.00 | 14.70 |
| ATOM | 4142 | CA | PHE | 619 | −3.131 | 13.280 | −14.213 | 1.00 | 13.63 |
| ATOM | 4143 | CB | PHE | 619 | −2.797 | 14.341 | −13.158 | 1.00 | 14.26 |
| ATOM | 4144 | CG | PHE | 619 | −3.688 | 14.296 | −11.951 | 1.00 | 15.87 |
| ATOM | 4145 | CD1 | PHE | 619 | −3.411 | 13.431 | −10.894 | 1.00 | 14.85 |
| ATOM | 4146 | CD2 | PHE | 619 | −4.816 | 15.109 | −11.875 | 1.00 | 17.83 |
| ATOM | 4147 | CE1 | PHE | 619 | −4.244 | 13.374 | −9.778 | 1.00 | 14.51 |
| ATOM | 4148 | CE2 | PHE | 619 | −5.657 | 15.062 | −10.764 | 1.00 | 17.27 |
| ATOM | 4149 | CZ | PHE | 619 | −5.370 | 14.191 | −9.711 | 1.00 | 15.64 |
| ATOM | 4150 | C | PHE | 619 | −2.815 | 11.896 | −13.671 | 1.00 | 15.10 |
| ATOM | 4151 | O | PHE | 619 | −3.660 | 11.258 | −13.051 | 1.00 | 14.55 |
| ATOM | 4152 | N | ALA | 620 | −1.586 | 11.451 | −13.903 | 1.00 | 13.79 |
| ATOM | 4153 | CA | ALA | 620 | −1.144 | 10.144 | −13.440 | 1.00 | 14.57 |
| ATOM | 4154 | CB | ALA | 620 | −0.186 | 9.529 | −14.460 | 1.00 | 14.31 |
| ATOM | 4155 | C | ALA | 620 | −0.482 | 10.194 | −12.067 | 1.00 | 14.58 |
| ATOM | 4156 | O | ALA | 620 | 0.149 | 11.185 | −11.687 | 1.00 | 13.79 |
| ATOM | 4157 | N | THR | 621 | −0.646 | 9.106 | −11.322 | 1.00 | 12.87 |
| ATOM | 4158 | CA | THR | 621 | −0.073 | 8.962 | −9.994 | 1.00 | 14.62 |
| ATOM | 4159 | CB | THR | 621 | −1.145 | 9.145 | −8.912 | 1.00 | 18.68 |
| ATOM | 4160 | OG1 | THR | 621 | −1.660 | 10.482 | −8.982 | 1.00 | 22.56 |
| ATOM | 4161 | CG2 | THR | 621 | −0.567 | 8.893 | −7.550 | 1.00 | 25.02 |
| ATOM | 4162 | C | THR | 621 | 0.457 | 7.538 | −9.925 | 1.00 | 13.44 |
| ATOM | 4163 | O | THR | 621 | −0.023 | 6.658 | −10.643 | 1.00 | 13.17 |
| ATOM | 4164 | N | ILE | 622 | 1.428 | 7.296 | −9.059 | 1.00 | 14.11 |
| ATOM | 4165 | CA | ILE | 622 | 1.984 | 5.958 | −8.979 | 1.00 | 14.52 |
| ATOM | 4166 | CB | ILE | 622 | 3.165 | 5.825 | −9.985 | 1.00 | 16.11 |
| ATOM | 4167 | CG2 | ILE | 622 | 4.372 | 6.630 | −9.495 | 1.00 | 19.85 |
| ATOM | 4168 | CG1 | ILE | 622 | 3.500 | 4.352 | −10.204 | 1.00 | 19.83 |
| ATOM | 4169 | CD1 | ILE | 622 | 4.468 | 4.118 | −11.347 | 1.00 | 22.61 |
| ATOM | 4170 | C | ILE | 622 | 2.443 | 5.606 | −7.570 | 1.00 | 13.66 |
| ATOM | 4171 | O | ILE | 622 | 2.596 | 6.481 | −6.735 | 1.00 | 12.92 |
| ATOM | 4172 | N | THR | 623 | 2.611 | 4.317 | −7.295 | 1.00 | 15.34 |
| ATOM | 4173 | CA | THR | 623 | 3.080 | 3.914 | −5.979 | 1.00 | 16.08 |
| ATOM | 4174 | CB | THR | 623 | 2.611 | 2.491 | −5.595 | 1.00 | 16.42 |
| ATOM | 4175 | OG1 | THR | 623 | 3.187 | 1.540 | −6.498 | 1.00 | 19.41 |
| ATOM | 4176 | CG2 | THR | 623 | 1.090 | 2.390 | −5.656 | 1.00 | 19.47 |
| ATOM | 4177 | C | THR | 623 | 4.602 | 3.910 | −6.063 | 1.00 | 14.73 |
| ATOM | 4178 | O | THR | 623 | 5.162 | 3.799 | −7.150 | 1.00 | 14.80 |
| ATOM | 4179 | N | ALA | 624 | 5.260 | 4.030 | −4.913 | 1.00 | 14.62 |
| ATOM | 4180 | CA | ALA | 624 | 6.719 | 4.024 | −4.826 | 1.00 | 14.05 |
| ATOM | 4181 | CB | ALA | 624 | 7.282 | 5.398 | −5.187 | 1.00 | 13.19 |
| ATOM | 4182 | C | ALA | 624 | 7.081 | 3.680 | −3.384 | 1.00 | 14.67 |
| ATOM | 4183 | O | ALA | 624 | 6.417 | 4.135 | −2.452 | 1.00 | 14.07 |
| ATOM | 4184 | N | TYR | 625 | 8.126 | 2.885 | −3.186 | 1.00 | 14.04 |
| ATOM | 4185 | CA | TYR | 625 | 8.504 | 2.522 | −1.830 | 1.00 | 13.33 |
| ATOM | 4186 | CB | TYR | 625 | 7.992 | 1.125 | −1.478 | 1.00 | 13.65 |
| ATOM | 4187 | CG | TYR | 625 | 6.658 | 0.790 | −2.089 | 1.00 | 11.16 |
| ATOM | 4188 | CD1 | TYR | 625 | 6.587 | 0.065 | −3.277 | 1.00 | 13.08 |
| ATOM | 4189 | CE1 | TYR | 625 | 5.367 | −0.253 | −3.852 | 1.00 | 12.70 |
| ATOM | 4190 | CD2 | TYR | 625 | 5.467 | 1.199 | −1.488 | 1.00 | 11.45 |
| ATOM | 4191 | CE2 | TYR | 625 | 4.234 | 0.886 | −2.057 | 1.00 | 11.45 |
| ATOM | 4192 | CZ | TYR | 625 | 4.194 | 0.155 | −3.241 | 1.00 | 11.70 |
| ATOM | 4193 | OH | TYR | 625 | 2.993 | −0.200 | −3.795 | 1.00 | 13.12 |
| ATOM | 4194 | C | TYR | 625 | 9.999 | 2.555 | −1.624 | 1.00 | 14.79 |
| ATOM | 4195 | O | TYR | 625 | 10.500 | 2.009 | −0.640 | 1.00 | 12.38 |
| ATOM | 4196 | N | ASP | 626 | 10.721 | 3.177 | −2.549 | 1.00 | 14.45 |
| ATOM | 4197 | CA | ASP | 626 | 12.166 | 3.263 | −2.410 | 1.00 | 16.17 |
| ATOM | 4198 | CB | ASP | 626 | 12.837 | 1.949 | −2.862 | 1.00 | 18.23 |
| ATOM | 4199 | CG | ASP | 626 | 12.721 | 1.703 | −4.362 | 1.00 | 19.53 |
| ATOM | 4200 | OD1 | ASP | 626 | 13.387 | 2.419 | −5.136 | 1.00 | 19.59 |
| ATOM | 4201 | OD2 | ASP | 626 | 11.964 | 0.792 | −4.764 | 1.00 | 21.03 |
| ATOM | 4202 | C | ASP | 626 | 12.746 | 4.454 | −3.159 | 1.00 | 16.01 |
| ATOM | 4203 | O | ASP | 626 | 12.091 | 5.068 | −4.009 | 1.00 | 15.86 |
| ATOM | 4204 | N | TYR | 627 | 13.989 | 4.771 | −2.826 | 1.00 | 15.06 |
| ATOM | 4205 | CA | TYR | 627 | 14.695 | 5.896 | −3.419 | 1.00 | 15.15 |
| ATOM | 4206 | CB | TYR | 627 | 16.058 | 6.039 | −2.745 | 1.00 | 16.61 |
| ATOM | 4207 | CG | TYR | 627 | 16.991 | 7.003 | −3.440 | 1.00 | 17.55 |
| ATOM | 4208 | CD1 | TYR | 627 | 17.025 | 8.351 | −3.089 | 1.00 | 17.88 |
| ATOM | 4209 | CE1 | TYR | 627 | 17.901 | 9.237 | −3.714 | 1.00 | 18.86 |
| ATOM | 4210 | CD2 | TYR | 627 | 17.854 | 6.561 | −4.441 | 1.00 | 18.63 |
| ATOM | 4211 | CE2 | TYR | 627 | 18.728 | 7.435 | −5.073 | 1.00 | 20.28 |
| ATOM | 4212 | CZ | TYR | 627 | 18.746 | 8.763 | −4.703 | 1.00 | 21.00 |
| ATOM | 4213 | OH | TYR | 627 | 19.624 | 9.615 | −5.330 | 1.00 | 23.89 |
| ATOM | 4214 | C | TYR | 627 | 14.892 | 5.816 | −4.929 | 1.00 | 15.66 |
| ATOM | 4215 | O | TYR | 627 | 14.681 | 6.797 | −5.635 | 1.00 | 15.39 |
| ATOM | 4216 | N | SER | 628 | 15.300 | 4.653 | −5.422 | 1.00 | 15.60 |
| ATOM | 4217 | CA | SER | 628 | 15.568 | 4.504 | −6.842 | 1.00 | 16.14 |
| ATOM | 4218 | CB | SER | 628 | 16.196 | 3.139 | −7.111 | 1.00 | 16.61 |
| ATOM | 4219 | OG | SER | 628 | 17.466 | 3.068 | −6.473 | 1.00 | 18.25 |
| ATOM | 4220 | C | SER | 628 | 14.367 | 4.733 | −7.746 | 1.00 | 16.64 |
| ATOM | 4221 | O | SER | 628 | 14.448 | 5.505 | −8.702 | 1.00 | 14.62 |
| ATOM | 4222 | N | PHE | 629 | 13.249 | 4.081 | −7.462 | 1.00 | 16.10 |
| ATOM | 4223 | CA | PHE | 629 | 12.090 | 4.303 | −8.307 | 1.00 | 15.35 |
| ATOM | 4224 | CB | PHE | 629 | 11.032 | 3.225 | −8.069 | 1.00 | 15.85 |
| ATOM | 4225 | CG | PHE | 629 | 11.350 | 1.934 | −8.769 | 1.00 | 16.27 |
| ATOM | 4226 | CD1 | PHE | 629 | 12.022 | 0.910 | −8.108 | 1.00 | 15.67 |
| ATOM | 4227 | CD2 | PHE | 629 | 11.039 | 1.774 | −10.118 | 1.00 | 14.97 |
| ATOM | 4228 | CE1 | PHE | 629 | 12.381 | −0.261 | −8.781 | 1.00 | 16.45 |
| ATOM | 4229 | CE2 | PHE | 629 | 11.391 | 0.616 | −10.803 | 1.00 | 15.51 |
| ATOM | 4230 | CZ | PHE | 629 | 12.066 | −0.408 | −10.137 | 1.00 | 15.52 |
| ATOM | 4231 | C | PHE | 629 | 11.526 | 5.712 | −8.131 | 1.00 | 15.42 |
| ATOM | 4232 | O | PHE | 629 | 11.149 | 6.362 | −9.110 | 1.00 | 14.31 |
| ATOM | 4233 | N | ALA | 630 | 11.509 | 6.210 | −6.900 | 1.00 | 13.96 |
| ATOM | 4234 | CA | ALA | 630 | 10.998 | 7.554 | −6.654 | 1.00 | 13.58 |
| ATOM | 4235 | CB | ALA | 630 | 11.058 | 7.872 | −5.164 | 1.00 | 15.22 |
| ATOM | 4236 | C | ALA | 630 | 11.800 | 8.585 | −7.447 | 1.00 | 14.75 |
| ATOM | 4237 | O | ALA | 630 | 11.240 | 9.526 | −8.024 | 1.00 | 14.71 |
| ATOM | 4238 | N | LYS | 631 | 13.115 | 8.408 | −7.467 | 1.00 | 15.76 |
| ATOM | 4239 | CA | LYS | 631 | 14.011 | 9.312 | −8.193 | 1.00 | 17.34 |
| ATOM | 4240 | CB | LYS | 631 | 15.465 | 8.898 | −7.923 | 1.00 | 17.65 |
| ATOM | 4241 | CG | LYS | 631 | 16.529 | 9.653 | −8.706 | 1.00 | 23.27 |
| ATOM | 4242 | CD | LYS | 631 | 16.611 | 11.105 | −8.294 | 1.00 | 24.41 |
| ATOM | 4243 | CE | LYS | 631 | 17.957 | 11.707 | −8.699 | 1.00 | 28.34 |
| ATOM | 4244 | NZ | LYS | 631 | 18.224 | 11.574 | −10.164 | 1.00 | 29.72 |
| ATOM | 4245 | C | LYS | 631 | 13.703 | 9.244 | −9.689 | 1.00 | 16.69 |
| ATOM | 4246 | O | LYS | 631 | 13.628 | 10.269 | −10.375 | 1.00 | 17.77 |
| ATOM | 4247 | N | LEU | 632 | 13.527 | 8.028 | −10.193 | 1.00 | 15.74 |
| ATOM | 4248 | CA | LEU | 632 | 13.224 | 7.828 | −11.606 | 1.00 | 14.84 |
| ATOM | 4249 | CB | LEU | 632 | 13.153 | 6.334 | −11.916 | 1.00 | 15.29 |
| ATOM | 4250 | CG | LEU | 632 | 13.100 | 5.918 | −13.390 | 1.00 | 14.76 |
| ATOM | 4251 | CD1 | LEU | 632 | 13.590 | 4.496 | −13.492 | 1.00 | 13.45 |
| ATOM | 4252 | CD2 | LEU | 632 | 11.689 | 6.064 | −13.954 | 1.00 | 14.18 |
| ATOM | 4253 | C | LEU | 632 | 11.909 | 8.503 | −11.997 | 1.00 | 16.72 |
| ATOM | 4254 | O | LEU | 632 | 11.834 | 9.159 | −13.035 | 1.00 | 15.36 |
| ATOM | 4255 | N | PHE | 633 | 10.872 | 8.335 | −11.177 | 1.00 | 15.97 |
| ATOM | 4256 | CA | PHE | 633 | 9.581 | 8.942 | −11.477 | 1.00 | 17.44 |
| ATOM | 4257 | CB | PHE | 633 | 8.497 | 8.463 | −10.493 | 1.00 | 16.12 |
| ATOM | 4258 | CG | PHE | 633 | 8.333 | 6.958 | −10.425 | 1.00 | 17.21 |
| ATOM | 4259 | CD1 | PHE | 633 | 8.562 | 6.156 | −11.542 | 1.00 | 18.33 |
| ATOM | 4260 | CD2 | PHE | 633 | 7.921 | 6.350 | −9.240 | 1.00 | 18.31 |
| ATOM | 4261 | CE1 | PHE | 633 | 8.386 | 4.763 | −11.483 | 1.00 | 17.53 |
| ATOM | 4262 | CE2 | PHE | 633 | 7.739 | 4.966 | −9.167 | 1.00 | 14.83 |
| ATOM | 4263 | CZ | PHE | 633 | 7.974 | 4.171 | −10.291 | 1.00 | 18.33 |
| ATOM | 4264 | C | PHE | 633 | 9.683 | 10.464 | −11.409 | 1.00 | 19.16 |
| ATOM | 4265 | O | PHE | 633 | 9.128 | 11.173 | −12.251 | 1.00 | 20.23 |
| ATOM | 4266 | N | ALA | 634 | 10.390 | 10.964 | −10.402 | 1.00 | 18.95 |
| ATOM | 4267 | CA | ALA | 634 | 10.561 | 12.402 | −10.235 | 1.00 | 20.80 |
| ATOM | 4268 | CB | ALA | 634 | 11.315 | 12.697 | −8.941 | 1.00 | 22.92 |
| ATOM | 4269 | C | ALA | 634 | 11.314 | 12.996 | −11.423 | 1.00 | 21.47 |
| ATOM | 4270 | O | ALA | 634 | 10.970 | 14.073 | −11.903 | 1.00 | 21.11 |
| ATOM | 4271 | N | ASP | 635 | 12.339 | 12.296 | −11.898 | 1.00 | 21.14 |
| ATOM | 4272 | CA | ASP | 635 | 13.113 | 12.792 | −13.030 | 1.00 | 23.15 |
| ATOM | 4273 | CB | ASP | 635 | 14.366 | 11.943 | −13.249 | 1.00 | 24.84 |
| ATOM | 4274 | CG | ASP | 635 | 15.388 | 12.102 | −12.141 | 1.00 | 24.40 |
| ATOM | 4275 | OD1 | ASP | 635 | 15.242 | 13.015 | −11.300 | 1.00 | 27.97 |
| ATOM | 4276 | OD2 | ASP | 635 | 16.349 | 11.311 | −12.127 | 1.00 | 26.01 |
| ATOM | 4277 | C | ASP | 635 | 12.319 | 12.844 | −14.338 | 1.00 | 23.85 |
| ATOM | 4278 | O | ASP | 635 | 12.662 | 13.606 | −15.247 | 1.00 | 23.95 |
| ATOM | 4279 | N | GLU | 636 | 11.269 | 12.034 | −14.434 | 1.00 | 22.21 |
| ATOM | 4280 | CA | GLU | 636 | 10.432 | 11.986 | −15.629 | 1.00 | 22.88 |
| ATOM | 4281 | CB | GLU | 636 | 9.971 | 10.555 | −15.899 | 1.00 | 22.80 |
| ATOM | 4282 | CG | GLU | 636 | 11.087 | 9.593 | −16.233 | 1.00 | 24.66 |
| ATOM | 4283 | CD | GLU | 636 | 11.863 | 10.024 | −17.459 | 1.00 | 25.48 |
| ATOM | 4284 | OE1 | GLU | 636 | 11.236 | 10.200 | −18.523 | 1.00 | 28.84 |
| ATOM | 4285 | OE2 | GLU | 636 | 13.092 | 10.186 | −17.359 | 1.00 | 26.19 |
| ATOM | 4286 | C | GLU | 636 | 9.203 | 12.879 | −15.543 | 1.00 | 21.66 |
| ATOM | 4287 | O | GLU | 636 | 8.483 | 13.035 | −16.528 | 1.00 | 22.31 |
| ATOM | 4288 | N | GLY | 637 | 8.939 | 13.435 | −14.365 | 1.00 | 20.53 |
| ATOM | 4289 | CA | GLY | 637 | 7.789 | 14.308 | −14.223 | 1.00 | 20.79 |
| ATOM | 4290 | C | GLY | 637 | 6.590 | 13.755 | −13.472 | 1.00 | 21.60 |
| ATOM | 4291 | O | GLY | 637 | 5.582 | 14.452 | −13.348 | 1.00 | 22.27 |
| ATOM | 4292 | N | LEU | 638 | 6.680 | 12.511 | −13.000 | 1.00 | 21.20 |
| ATOM | 4293 | CA | LEU | 638 | 5.599 | 11.891 | −12.228 | 1.00 | 22.21 |
| ATOM | 4294 | CB | LEU | 638 | 5.683 | 10.361 | −12.301 | 1.00 | 23.81 |
| ATOM | 4295 | CG | LEU | 638 | 4.426 | 9.629 | −12.774 | 1.00 | 26.98 |
| ATOM | 4296 | CD1 | LEU | 638 | 4.678 | 8.133 | −12.758 | 1.00 | 25.19 |
| ATOM | 4297 | CD2 | LEU | 638 | 3.241 | 9.988 | −11.903 | 1.00 | 27.34 |
| ATOM | 4298 | C | LEU | 638 | 5.876 | 12.355 | −10.807 | 1.00 | 20.11 |
| ATOM | 4299 | O | LEU | 638 | 6.683 | 11.752 | −10.094 | 1.00 | 22.26 |
| ATOM | 4300 | N | ASN | 639 | 5.203 | 13.427 | −10.409 | 1.00 | 20.38 |
| ATOM | 4301 | CA | ASN | 639 | 5.415 | 14.035 | −9.105 | 1.00 | 18.91 |
| ATOM | 4302 | CB | ASN | 639 | 5.557 | 15.555 | −9.264 | 1.00 | 24.25 |
| ATOM | 4303 | CG | ASN | 639 | 6.571 | 15.944 | −10.327 | 1.00 | 28.84 |
| ATOM | 4304 | OD1 | ASN | 639 | 7.684 | 15.416 | −10.361 | 1.00 | 31.74 |
| ATOM | 4305 | ND2 | ASN | 639 | 6.195 | 16.881 | −11.194 | 1.00 | 31.59 |
| ATOM | 4306 | C | ASN | 639 | 4.348 | 13.747 | −8.054 | 1.00 | 17.68 |
| ATOM | 4307 | O | ASN | 639 | 4.240 | 14.470 | −7.061 | 1.00 | 16.28 |
| ATOM | 4308 | N | VAL | 640 | 3.547 | 12.711 | −8.265 | 1.00 | 16.43 |
| ATOM | 4309 | CA | VAL | 640 | 2.518 | 12.366 | −7.289 | 1.00 | 15.98 |
| ATOM | 4310 | CB | VAL | 640 | 1.101 | 12.590 | −7.840 | 1.00 | 16.39 |
| ATOM | 4311 | CG1 | VAL | 640 | 0.083 | 12.203 | −6.787 | 1.00 | 18.52 |
| ATOM | 4312 | CG2 | VAL | 640 | 0.917 | 14.054 | −8.224 | 1.00 | 18.53 |
| ATOM | 4313 | C | VAL | 640 | 2.712 | 10.895 | −6.973 | 1.00 | 15.29 |
| ATOM | 4314 | O | VAL | 640 | 2.445 | 10.036 | −7.811 | 1.00 | 11.74 |
| ATOM | 4315 | N | MET | 641 | 3.190 | 10.618 | −5.764 | 1.00 | 13.95 |
| ATOM | 4316 | CA | MET | 641 | 3.477 | 9.252 | −5.362 | 1.00 | 14.10 |
| ATOM | 4317 | CB | MET | 641 | 4.989 | 9.052 | −5.237 | 1.00 | 14.03 |
| ATOM | 4318 | CG | MET | 641 | 5.725 | 9.242 | −6.546 | 1.00 | 18.23 |
| ATOM | 4319 | SD | MET | 641 | 7.481 | 9.167 | −6.340 | 1.00 | 18.97 |
| ATOM | 4320 | CE | MET | 641 | 8.019 | 10.537 | −7.397 | 1.00 | 20.65 |
| ATOM | 4321 | C | MET | 641 | 2.815 | 8.827 | −4.076 | 1.00 | 12.89 |
| ATOM | 4322 | O | MET | 641 | 2.704 | 9.599 | −3.127 | 1.00 | 16.15 |
| ATOM | 4323 | N | LEU | 642 | 2.405 | 7.569 | −4.041 | 1.00 | 13.58 |
| ATOM | 4324 | CA | LEU | 642 | 1.743 | 7.045 | −2.865 | 1.00 | 15.28 |
| ATOM | 4325 | CB | LEU | 642 | 0.330 | 6.602 | −3.257 | 1.00 | 19.13 |
| ATOM | 4326 | CG | LEU | 642 | −0.652 | 5.954 | −2.276 | 1.00 | 23.45 |
| ATOM | 4327 | CD1 | LEU | 642 | −0.395 | 4.474 | −2.248 | 1.00 | 25.41 |
| ATOM | 4328 | CD2 | LEU | 642 | −0.555 | 6.576 | −0.881 | 1.00 | 22.82 |
| ATOM | 4329 | C | LEU | 642 | 2.533 | 5.908 | −2.230 | 1.00 | 15.23 |
| ATOM | 4330 | O | LEU | 642 | 2.920 | 4.947 | −2.898 | 1.00 | 15.25 |
| ATOM | 4331 | N | VAL | 643 | 2.782 | 6.053 | −0.933 | 1.00 | 16.09 |
| ATOM | 4332 | CA | VAL | 643 | 3.478 | 5.037 | −0.155 | 1.00 | 16.71 |
| ATOM | 4333 | CB | VAL | 643 | 4.389 | 5.668 | 0.917 | 1.00 | 15.65 |
| ATOM | 4334 | CG1 | VAL | 643 | 5.181 | 4.576 | 1.632 | 1.00 | 17.25 |
| ATOM | 4335 | CG2 | VAL | 643 | 5.351 | 6.663 | 0.261 | 1.00 | 20.05 |
| ATOM | 4336 | C | VAL | 643 | 2.327 | 4.297 | 0.508 | 1.00 | 15.14 |
| ATOM | 4337 | O | VAL | 643 | 1.931 | 4.624 | 1.624 | 1.00 | 15.03 |
| ATOM | 4338 | N | GLY | 644 | 1.784 | 3.313 | −0.206 | 1.00 | 18.13 |
| ATOM | 4339 | CA | GLY | 644 | 0.646 | 2.565 | 0.288 | 1.00 | 16.80 |
| ATOM | 4340 | C | GLY | 644 | 0.948 | 1.249 | 0.963 | 1.00 | 16.33 |
| ATOM | 4341 | O | GLY | 644 | 2.038 | 0.698 | 0.802 | 1.00 | 15.86 |
| ATOM | 4342 | N | ASP | 645 | −0.020 | 0.735 | 1.717 | 1.00 | 15.60 |
| ATOM | 4343 | CA | ASP | 645 | 0.200 | −0.529 | 2.411 | 1.00 | 16.74 |
| ATOM | 4344 | CB | ASP | 645 | −0.837 | −0.755 | 3.521 | 1.00 | 15.16 |
| ATOM | 4345 | CG | ASP | 645 | −2.268 | −0.790 | 3.017 | 1.00 | 16.47 |
| ATOM | 4346 | OD1 | ASP | 645 | −2.499 | −0.772 | 1.794 | 1.00 | 12.97 |
| ATOM | 4347 | OD2 | ASP | 645 | −3.178 | −0.843 | 3.875 | 1.00 | 16.97 |
| ATOM | 4348 | C | ASP | 645 | 0.258 | −1.703 | 1.446 | 1.00 | 15.61 |
| ATOM | 4349 | O | ASP | 645 | 0.406 | −2.856 | 1.860 | 1.00 | 18.52 |
| ATOM | 4350 | N | SER | 646 | 0.169 | −1.399 | 0.151 | 1.00 | 14.45 |
| ATOM | 4351 | CA | SER | 646 | 0.283 | −2.425 | −0.879 | 1.00 | 12.93 |
| ATOM | 4352 | CB | SER | 646 | 0.062 | −1.821 | −2.266 | 1.00 | 14.70 |
| ATOM | 4353 | OG | SER | 646 | 0.943 | −0.726 | −2.478 | 1.00 | 16.74 |
| ATOM | 4354 | C | SER | 646 | 1.695 | −2.994 | −0.801 | 1.00 | 13.99 |
| ATOM | 4355 | O | SER | 646 | 1.969 | −4.085 | −1.308 | 1.00 | 15.91 |
| ATOM | 4356 | N | LEU | 647 | 2.596 | −2.248 | −0.170 | 1.00 | 11.90 |
| ATOM | 4357 | CA | LEU | 647 | 3.977 | −2.702 | −0.031 | 1.00 | 12.24 |
| ATOM | 4358 | CB | LEU | 647 | 4.839 | −1.599 | 0.601 | 1.00 | 12.62 |
| ATOM | 4359 | CG | LEU | 647 | 4.542 | −1.162 | 2.038 | 1.00 | 13.54 |
| ATOM | 4360 | CD1 | LEU | 647 | 5.201 | −2.120 | 3.026 | 1.00 | 15.69 |
| ATOM | 4361 | CD2 | LEU | 647 | 5.062 | 0.254 | 2.247 | 1.00 | 14.93 |
| ATOM | 4362 | C | LEU | 647 | 4.015 | −3.975 | 0.818 | 1.00 | 12.53 |
| ATOM | 4363 | O | LEU | 647 | 4.983 | −4.728 | 0.765 | 1.00 | 13.66 |
| ATOM | 4364 | N | GLY | 648 | 2.954 | −4.216 | 1.586 | 1.00 | 12.76 |
| ATOM | 4365 | CA | GLY | 648 | 2.910 | −5.410 | 2.417 | 1.00 | 12.76 |
| ATOM | 4366 | C | GLY | 648 | 2.941 | −6.654 | 1.554 | 1.00 | 13.52 |
| ATOM | 4367 | O | GLY | 648 | 3.395 | −7.720 | 1.973 | 1.00 | 12.64 |
| ATOM | 4368 | N | MET | 649 | 2.456 | −6.510 | 0.328 | 1.00 | 13.40 |
| ATOM | 4369 | CA | MET | 649 | 2.434 | −7.615 | −0.611 | 1.00 | 13.82 |
| ATOM | 4370 | CB | MET | 649 | 1.068 | −7.672 | −1.307 | 1.00 | 15.15 |
| ATOM | 4371 | CG | MET | 649 | −0.080 | −7.870 | −0.326 | 1.00 | 19.61 |
| ATOM | 4372 | SD | MET | 649 | −1.749 | −7.781 | −1.021 | 1.00 | 23.07 |
| ATOM | 4373 | CE | MET | 649 | −1.676 | −9.060 | −2.275 | 1.00 | 19.40 |
| ATOM | 4374 | C | MET | 649 | 3.563 | −7.497 | −1.631 | 1.00 | 14.32 |
| ATOM | 4375 | O | MET | 649 | 4.352 | −8.418 | −1.802 | 1.00 | 13.75 |
| ATOM | 4376 | N | THR | 650 | 3.678 | −6.353 | −2.290 | 1.00 | 12.15 |
| ATOM | 4377 | CA | THR | 650 | 4.717 | −6.216 | −3.303 | 1.00 | 13.72 |
| ATOM | 4378 | CB | THR | 650 | 4.416 | −5.015 | −4.221 | 1.00 | 15.77 |
| ATOM | 4379 | OG1 | THR | 650 | 4.506 | −3.803 | −3.475 | 1.00 | 21.01 |
| ATOM | 4380 | CG2 | THR | 650 | 3.011 | −5.143 | −4.785 | 1.00 | 15.50 |
| ATOM | 4381 | C | THR | 650 | 6.144 | −6.135 | −2.776 | 1.00 | 12.32 |
| ATOM | 4382 | O | THR | 650 | 7.089 | −6.565 | −3.445 | 1.00 | 11.07 |
| ATOM | 4383 | N | VAL | 651 | 6.310 | −5.595 | −1.576 | 1.00 | 11.93 |
| ATOM | 4384 | CA | VAL | 651 | 7.645 | −5.477 | −0.995 | 1.00 | 12.05 |
| ATOM | 4385 | CB | VAL | 651 | 7.845 | −4.079 | −0.358 | 1.00 | 12.18 |
| ATOM | 4386 | CG1 | VAL | 651 | 9.211 | −3.993 | 0.316 | 1.00 | 13.43 |
| ATOM | 4387 | CG2 | VAL | 651 | 7.711 | −3.005 | −1.423 | 1.00 | 13.62 |
| ATOM | 4388 | C | VAL | 651 | 7.895 | −6.557 | 0.057 | 1.00 | 11.83 |
| ATOM | 4389 | O | VAL | 651 | 8.858 | −7.316 | −0.035 | 1.00 | 12.12 |
| ATOM | 4390 | N | GLN | 652 | 7.018 | −6.640 | 1.049 | 1.00 | 12.39 |
| ATOM | 4391 | CA | GLN | 652 | 7.204 | −7.615 | 2.123 | 1.00 | 13.70 |
| ATOM | 4392 | CB | GLN | 652 | 6.420 | −7.177 | 3.350 | 1.00 | 13.40 |
| ATOM | 4393 | CG | GLN | 652 | 6.796 | −5.783 | 3.818 | 1.00 | 13.66 |
| ATOM | 4394 | CD | GLN | 652 | 6.004 | −5.362 | 5.028 | 1.00 | 13.82 |
| ATOM | 4395 | OE1 | GLN | 652 | 4.978 | −5.962 | 5.340 | 1.00 | 14.37 |
| ATOM | 4396 | NE2 | GLN | 652 | 6.464 | −4.319 | 5.713 | 1.00 | 9.59 |
| ATOM | 4397 | C | GLN | 652 | 6.845 | −9.053 | 1.770 | 1.00 | 13.88 |
| ATOM | 4398 | O | GLN | 652 | 7.356 | −9.982 | 2.388 | 1.00 | 14.21 |
| ATOM | 4399 | N | GLY | 653 | 5.957 | −9.239 | 0.799 | 1.00 | 12.48 |
| ATOM | 4400 | CA | GLY | 653 | 5.595 | −10.590 | 0.408 | 1.00 | 14.75 |
| ATOM | 4401 | C | GLY | 653 | 4.474 | −11.249 | 1.193 | 1.00 | 14.96 |
| ATOM | 4402 | O | GLY | 653 | 4.323 | −12.468 | 1.144 | 1.00 | 14.96 |
| ATOM | 4403 | N | HIS | 654 | 3.693 | −10.461 | 1.924 | 1.00 | 14.70 |
| ATOM | 4404 | CA | HIS | 654 | 2.572 | −11.008 | 2.678 | 1.00 | 18.41 |
| ATOM | 4405 | CB | HIS | 654 | 2.150 | −10.052 | 3.792 | 1.00 | 15.90 |
| ATOM | 4406 | CG | HIS | 654 | 3.191 | −9.857 | 4.844 | 1.00 | 17.16 |
| ATOM | 4407 | CD2 | HIS | 654 | 3.830 | −8.740 | 5.265 | 1.00 | 12.42 |
| ATOM | 4408 | ND1 | HIS | 654 | 3.677 | −10.892 | 5.614 | 1.00 | 15.54 |
| ATOM | 4409 | CE1 | HIS | 654 | 4.571 | −10.421 | 6.463 | 1.00 | 17.70 |
| ATOM | 4410 | NE2 | HIS | 654 | 4.683 | −9.118 | 6.272 | 1.00 | 15.36 |
| ATOM | 4411 | C | HIS | 654 | 1.387 | −11.248 | 1.745 | 1.00 | 18.82 |
| ATOM | 4412 | O | HIS | 654 | 1.349 | −10.723 | 0.631 | 1.00 | 20.21 |
| ATOM | 4413 | N | ASP | 655 | 0.420 | −12.034 | 2.216 | 1.00 | 19.49 |
| ATOM | 4414 | CA | ASP | 655 | −0.769 | −12.374 | 1.436 | 1.00 | 20.72 |
| ATOM | 4415 | CB | ASP | 655 | −1.406 | −13.663 | 1.978 | 1.00 | 25.65 |
| ATOM | 4416 | CG | ASP | 655 | −2.228 | −13.426 | 3.234 | 1.00 | 28.75 |
| ATOM | 4417 | OD1 | ASP | 655 | −3.324 | −12.830 | 3.133 | 1.00 | 36.14 |
| ATOM | 4418 | OD2 | ASP | 655 | −1.789 | −13.825 | 4.330 | 1.00 | 34.78 |
| ATOM | 4419 | C | ASP | 655 | −1.815 | −11.263 | 1.458 | 1.00 | 19.20 |
| ATOM | 4420 | O | ASP | 655 | −2.805 | −11.320 | 0.729 | 1.00 | 20.52 |
| ATOM | 4421 | N | SER | 656 | −1.610 | −10.267 | 2.310 | 1.00 | 16.01 |
| ATOM | 4422 | CA | SER | 656 | −2.545 | −9.149 | 2.407 | 1.00 | 14.62 |
| ATOM | 4423 | CB | SER | 656 | −3.663 | −9.467 | 3.402 | 1.00 | 14.60 |
| ATOM | 4424 | OG | SER | 656 | −3.203 | −9.327 | 4.731 | 1.00 | 13.62 |
| ATOM | 4425 | C | SER | 656 | −1.771 | −7.940 | 2.897 | 1.00 | 11.97 |
| ATOM | 4426 | O | SER | 656 | −0.579 | −8.031 | 3.185 | 1.00 | 11.43 |
| ATOM | 4427 | N | THR | 657 | −2.447 | −6.801 | 2.993 | 1.00 | 13.16 |
| ATOM | 4428 | CA | THR | 657 | −1.801 | −5.578 | 3.461 | 1.00 | 12.70 |
| ATOM | 4429 | CB | THR | 657 | −2.433 | −4.325 | 2.799 | 1.00 | 14.37 |
| ATOM | 4430 | OG1 | THR | 657 | −3.782 | −4.183 | 3.240 | 1.00 | 14.29 |
| ATOM | 4431 | CG2 | THR | 657 | −2.421 | −4.441 | 1.279 | 1.00 | 16.57 |
| ATOM | 4432 | C | THR | 657 | −1.852 | −5.394 | 4.980 | 1.00 | 12.71 |
| ATOM | 4433 | O | THR | 657 | −1.148 | −4.550 | 5.519 | 1.00 | 13.29 |
| ATOM | 4434 | N | LEU | 658 | −2.675 | −6.177 | 5.675 | 1.00 | 14.03 |
| ATOM | 4435 | CA | LEU | 658 | −2.823 | −6.012 | 7.135 | 1.00 | 13.77 |
| ATOM | 4436 | CB | LEU | 658 | −3.780 | −7.076 | 7.692 | 1.00 | 15.46 |
| ATOM | 4437 | CG | LEU | 658 | −5.279 | −6.806 | 7.474 | 1.00 | 17.50 |
| ATOM | 4438 | CD1 | LEU | 658 | −5.634 | −7.040 | 6.012 | 1.00 | 21.01 |
| ATOM | 4439 | CD2 | LEU | 658 | −6.099 | −7.729 | 8.372 | 1.00 | 18.31 |
| ATOM | 4440 | C | LEU | 658 | −1.561 | −5.951 | 8.003 | 1.00 | 12.69 |
| ATOM | 4441 | O | LEU | 658 | −1.485 | −5.150 | 8.927 | 1.00 | 12.58 |
| ATOM | 4442 | N | PRO | 659 | −0.558 | −6.799 | 7.722 | 1.00 | 14.97 |
| ATOM | 4443 | CD | PRO | 659 | −0.581 | −7.923 | 6.772 | 1.00 | 12.48 |
| ATOM | 4444 | CA | PRO | 659 | 0.685 | −6.810 | 8.501 | 1.00 | 12.86 |
| ATOM | 4445 | CB | PRO | 659 | 1.425 | −8.038 | 7.957 | 1.00 | 14.97 |
| ATOM | 4446 | CG | PRO | 659 | 0.870 | −8.175 | 6.570 | 1.00 | 21.34 |
| ATOM | 4447 | C | PRO | 659 | 1.540 | −5.546 | 8.448 | 1.00 | 13.36 |
| ATOM | 4448 | O | PRO | 659 | 2.434 | −5.365 | 9.273 | 1.00 | 14.03 |
| ATOM | 4449 | N | VAL | 660 | 1.271 | −4.668 | 7.487 | 1.00 | 13.75 |
| ATOM | 4450 | CA | VAL | 660 | 2.031 | −3.427 | 7.358 | 1.00 | 12.32 |
| ATOM | 4451 | CB | VAL | 660 | 1.619 | −2.672 | 6.069 | 1.00 | 12.31 |
| ATOM | 4452 | CG1 | VAL | 660 | 2.316 | −1.318 | 5.990 | 1.00 | 13.70 |
| ATOM | 4453 | CG2 | VAL | 660 | 1.962 | −3.515 | 4.868 | 1.00 | 10.14 |
| ATOM | 4454 | C | VAL | 660 | 1.794 | −2.536 | 8.563 | 1.00 | 13.42 |
| ATOM | 4455 | O | VAL | 660 | 0.649 | −2.261 | 8.936 | 1.00 | 14.34 |
| ATOM | 4456 | N | THR | 661 | 2.878 | −2.077 | 9.176 | 1.00 | 11.86 |
| ATOM | 4457 | CA | THR | 661 | 2.772 | −1.221 | 10.357 | 1.00 | 15.36 |
| ATOM | 4458 | CB | THR | 661 | 3.795 | −1.640 | 11.439 | 1.00 | 17.87 |
| ATOM | 4459 | OG1 | THR | 661 | 3.640 | −3.039 | 11.736 | 1.00 | 21.53 |
| ATOM | 4460 | CG2 | THR | 661 | 3.569 | −0.835 | 12.720 | 1.00 | 22.77 |
| ATOM | 4461 | C | THR | 661 | 3.051 | 0.233 | 9.998 | 1.00 | 14.14 |
| ATOM | 4462 | O | THR | 661 | 3.553 | 0.517 | 8.916 | 1.00 | 11.93 |
| ATOM | 4463 | N | VAL | 662 | 2.722 | 1.141 | 10.912 | 1.00 | 13.31 |
| ATOM | 4464 | CA | VAL | 662 | 2.969 | 2.560 | 10.712 | 1.00 | 15.63 |
| ATOM | 4465 | CB | VAL | 662 | 2.465 | 3.395 | 11.912 | 1.00 | 15.72 |
| ATOM | 4466 | CG1 | VAL | 662 | 3.002 | 4.809 | 11.832 | 1.00 | 18.60 |
| ATOM | 4467 | CG2 | VAL | 662 | 0.938 | 3.412 | 11.921 | 1.00 | 15.56 |
| ATOM | 4468 | C | VAL | 662 | 4.471 | 2.758 | 10.556 | 1.00 | 15.26 |
| ATOM | 4469 | O | VAL | 662 | 4.913 | 3.566 | 9.740 | 1.00 | 15.83 |
| ATOM | 4470 | N | ALA | 663 | 5.255 | 2.004 | 11.323 | 1.00 | 14.05 |
| ATOM | 4471 | CA | ALA | 663 | 6.707 | 2.100 | 11.236 | 1.00 | 14.62 |
| ATOM | 4472 | CB | ALA | 663 | 7.364 | 1.189 | 12.278 | 1.00 | 15.71 |
| ATOM | 4473 | C | ALA | 663 | 7.178 | 1.733 | 9.830 | 1.00 | 14.13 |
| ATOM | 4474 | O | ALA | 663 | 8.092 | 2.370 | 9.302 | 1.00 | 13.75 |
| ATOM | 4475 | N | ASP | 664 | 6.556 | 0.729 | 9.210 | 1.00 | 14.22 |
| ATOM | 4476 | CA | ASP | 664 | 6.966 | 0.354 | 7.849 | 1.00 | 13.17 |
| ATOM | 4477 | CB | ASP | 664 | 6.232 | −0.886 | 7.327 | 1.00 | 13.69 |
| ATOM | 4478 | CG | ASP | 664 | 6.434 | −2.115 | 8.200 | 1.00 | 11.91 |
| ATOM | 4479 | OD1 | ASP | 664 | 7.525 | −2.275 | 8.780 | 1.00 | 10.98 |
| ATOM | 4480 | OD2 | ASP | 664 | 5.494 | −2.925 | 8.257 | 1.00 | 14.69 |
| ATOM | 4481 | C | ASP | 664 | 6.650 | 1.494 | 6.895 | 1.00 | 12.45 |
| ATOM | 4482 | O | ASP | 664 | 7.472 | 1.838 | 6.047 | 1.00 | 10.45 |
| ATOM | 4483 | N | ILE | 665 | 5.454 | 2.067 | 7.015 | 1.00 | 11.31 |
| ATOM | 4484 | CA | ILE | 665 | 5.088 | 3.165 | 6.126 | 1.00 | 11.92 |
| ATOM | 4485 | CB | ILE | 665 | 3.680 | 3.717 | 6.429 | 1.00 | 13.42 |
| ATOM | 4486 | CG2 | ILE | 665 | 3.406 | 4.942 | 5.556 | 1.00 | 14.44 |
| ATOM | 4487 | CG1 | ILE | 665 | 2.621 | 2.645 | 6.162 | 1.00 | 11.06 |
| ATOM | 4488 | CD1 | ILE | 665 | 2.482 | 2.252 | 4.704 | 1.00 | 14.31 |
| ATOM | 4489 | C | ILE | 665 | 6.097 | 4.299 | 6.243 | 1.00 | 12.95 |
| ATOM | 4490 | O | ILE | 665 | 6.547 | 4.845 | 5.227 | 1.00 | 12.47 |
| ATOM | 4491 | N | ALA | 666 | 6.453 | 4.643 | 7.481 | 1.00 | 13.58 |
| ATOM | 4492 | CA | ALA | 666 | 7.406 | 5.725 | 7.764 | 1.00 | 13.36 |
| ATOM | 4493 | CB | ALA | 666 | 7.528 | 5.929 | 9.275 | 1.00 | 13.24 |
| ATOM | 4494 | C | ALA | 666 | 8.788 | 5.468 | 7.167 | 1.00 | 13.44 |
| ATOM | 4495 | O | ALA | 666 | 9.435 | 6.386 | 6.656 | 1.00 | 11.50 |
| ATOM | 4496 | N | TYR | 667 | 9.232 | 4.215 | 7.242 | 1.00 | 12.26 |
| ATOM | 4497 | CA | TYR | 667 | 10.528 | 3.806 | 6.703 | 1.00 | 12.09 |
| ATOM | 4498 | CB | TYR | 667 | 10.760 | 2.315 | 6.987 | 1.00 | 12.31 |
| ATOM | 4499 | CG | TYR | 667 | 12.002 | 1.719 | 6.345 | 1.00 | 11.83 |
| ATOM | 4500 | CD1 | TYR | 667 | 13.277 | 1.995 | 6.845 | 1.00 | 13.98 |
| ATOM | 4501 | CE1 | TYR | 667 | 14.418 | 1.421 | 6.275 | 1.00 | 14.36 |
| ATOM | 4502 | CD2 | TYR | 667 | 11.897 | 0.855 | 5.252 | 1.00 | 15.01 |
| ATOM | 4503 | CE2 | TYR | 667 | 13.027 | 0.280 | 4.673 | 1.00 | 13.37 |
| ATOM | 4504 | CZ | TYR | 667 | 14.286 | 0.568 | 5.189 | 1.00 | 15.02 |
| ATOM | 4505 | OH | TYR | 667 | 15.404 | 0.011 | 4.612 | 1.00 | 14.89 |
| ATOM | 4506 | C | TYR | 667 | 10.556 | 4.050 | 5.194 | 1.00 | 11.94 |
| ATOM | 4507 | O | TYR | 667 | 11.453 | 4.712 | 4.672 | 1.00 | 9.26 |
| ATOM | 4508 | N | HIS | 668 | 9.565 | 3.501 | 4.499 | 1.00 | 12.01 |
| ATOM | 4509 | CA | HIS | 668 | 9.473 | 3.670 | 3.057 | 1.00 | 13.41 |
| ATOM | 4510 | CB | HIS | 668 | 8.423 | 2.713 | 2.486 | 1.00 | 12.37 |
| ATOM | 4511 | CG | HIS | 668 | 8.849 | 1.278 | 2.536 | 1.00 | 11.83 |
| ATOM | 4512 | CD2 | HIS | 668 | 8.557 | 0.293 | 3.419 | 1.00 | 11.45 |
| ATOM | 4513 | ND1 | HIS | 668 | 9.774 | 0.746 | 1.663 | 1.00 | 13.13 |
| ATOM | 4514 | CE1 | HIS | 668 | 10.038 | −0.503 | 2.008 | 1.00 | 11.30 |
| ATOM | 4515 | NE2 | HIS | 668 | 9.312 | −0.801 | 3.071 | 1.00 | 11.79 |
| ATOM | 4516 | C | HIS | 668 | 9.159 | 5.117 | 2.682 | 1.00 | 14.60 |
| ATOM | 4517 | O | HIS | 668 | 9.641 | 5.618 | 1.662 | 1.00 | 16.39 |
| ATOM | 4518 | N | THR | 669 | 8.369 | 5.803 | 3.501 | 1.00 | 15.65 |
| ATOM | 4519 | CA | THR | 669 | 8.042 | 7.197 | 3.202 | 1.00 | 16.31 |
| ATOM | 4520 | CB | THR | 669 | 7.070 | 7.797 | 4.250 | 1.00 | 15.04 |
| ATOM | 4521 | OG1 | THR | 669 | 5.787 | 7.170 | 4.110 | 1.00 | 15.98 |
| ATOM | 4522 | CG2 | THR | 669 | 6.914 | 9.314 | 4.036 | 1.00 | 15.38 |
| ATOM | 4523 | C | THR | 669 | 9.313 | 8.046 | 3.133 | 1.00 | 15.41 |
| ATOM | 4524 | O | THR | 669 | 9.495 | 8.841 | 2.202 | 1.00 | 15.01 |
| ATOM | 4525 | N | ALA | 670 | 10.205 | 7.858 | 4.104 | 1.00 | 15.82 |
| ATOM | 4526 | CA | ALA | 670 | 11.458 | 8.615 | 4.151 | 1.00 | 14.91 |
| ATOM | 4527 | CB | ALA | 670 | 12.230 | 8.272 | 5.426 | 1.00 | 16.01 |
| ATOM | 4528 | C | ALA | 670 | 12.333 | 8.349 | 2.934 | 1.00 | 16.09 |
| ATOM | 4529 | O | ALA | 670 | 13.005 | 9.255 | 2.416 | 1.00 | 14.84 |
| ATOM | 4530 | N | ALA | 671 | 12.337 | 7.098 | 2.483 | 1.00 | 15.41 |
| ATOM | 4531 | CA | ALA | 671 | 13.141 | 6.713 | 1.327 | 1.00 | 15.41 |
| ATOM | 4532 | CB | ALA | 671 | 13.162 | 5.184 | 1.190 | 1.00 | 14.87 |
| ATOM | 4533 | C | ALA | 671 | 12.606 | 7.359 | 0.049 | 1.00 | 16.10 |
| ATOM | 4534 | O | ALA | 671 | 13.377 | 7.832 | −0.794 | 1.00 | 14.32 |
| ATOM | 4535 | N | VAL | 672 | 11.285 | 7.382 | −0.096 | 1.00 | 14.00 |
| ATOM | 4536 | CA | VAL | 672 | 10.677 | 8.000 | −1.269 | 1.00 | 14.46 |
| ATOM | 4537 | CB | VAL | 672 | 9.148 | 7.738 | −1.313 | 1.00 | 15.01 |
| ATOM | 4538 | CG1 | VAL | 672 | 8.482 | 8.651 | −2.351 | 1.00 | 15.50 |
| ATOM | 4539 | CG2 | VAL | 672 | 8.891 | 6.278 | −1.686 | 1.00 | 16.48 |
| ATOM | 4540 | C | VAL | 672 | 10.933 | 9.508 | −1.270 | 1.00 | 14.16 |
| ATOM | 4541 | O | VAL | 672 | 11.181 | 10.108 | −2.322 | 1.00 | 15.05 |
| ATOM | 4542 | N | ARG | 673 | 10.883 | 10.125 | −0.095 | 1.00 | 13.51 |
| ATOM | 4543 | CA | ARG | 673 | 11.109 | 11.564 | −0.010 | 1.00 | 15.06 |
| ATOM | 4544 | CB | ARG | 673 | 10.777 | 12.086 | 1.390 | 1.00 | 15.22 |
| ATOM | 4545 | CG | ARG | 673 | 11.038 | 13.580 | 1.570 | 1.00 | 16.26 |
| ATOM | 4546 | CD | ARG | 673 | 10.302 | 14.412 | 0.532 | 1.00 | 15.12 |
| ATOM | 4547 | NE | ARG | 673 | 8.877 | 14.529 | 0.829 | 1.00 | 16.62 |
| ATOM | 4548 | CZ | ARG | 673 | 7.983 | 15.058 | 0.001 | 1.00 | 17.11 |
| ATOM | 4549 | NH1 | ARG | 673 | 8.361 | 15.518 | −1.187 | 1.00 | 16.70 |
| ATOM | 4550 | NH2 | ARG | 673 | 6.712 | 15.137 | 0.361 | 1.00 | 15.79 |
| ATOM | 4551 | C | ARG | 673 | 12.335 | 11.937 | −0.384 | 1.00 | 16.05 |
| ATOM | 4552 | O | ARG | 673 | 12.764 | 12.998 | −0.963 | 1.00 | 16.70 |
| ATOM | 4553 | N | ARG | 674 | 13.490 | 11.072 | −0.050 | 1.00 | 15.93 |
| ATOM | 4554 | CA | ARG | 674 | 14.883 | 11.332 | −0.394 | 1.00 | 17.55 |
| ATOM | 4555 | CB | ARG | 674 | 15.814 | 10.311 | 0.279 | 1.00 | 17.79 |
| ATOM | 4556 | CG | ARG | 674 | 15.769 | 10.334 | 1.806 | 1.00 | 17.42 |
| ATOM | 4557 | CD | ARG | 674 | 16.944 | 9.589 | 2.437 | 1.00 | 19.60 |
| ATOM | 4558 | NE | ARG | 674 | 16.811 | 9.507 | 3.894 | 1.00 | 22.66 |
| ATOM | 4559 | CZ | ARG | 674 | 16.242 | 8.494 | 4.544 | 1.00 | 21.67 |
| ATOM | 4560 | NH1 | ARG | 674 | 15.755 | 7.458 | 3.874 | 1.00 | 19.18 |
| ATOM | 4561 | NH2 | ARG | 674 | 16.149 | 8.519 | 5.866 | 1.00 | 22.65 |
| ATOM | 4562 | C | ARG | 674 | 15.045 | 11.264 | −1.917 | 1.00 | 18.81 |
| ATOM | 4563 | O | ARG | 674 | 15.865 | 11.978 | −2.489 | 1.00 | 17.29 |
| ATOM | 4564 | N | GLY | 675 | 14.250 | 10.418 | −2.568 | 1.00 | 18.30 |
| ATOM | 4565 | CA | GLY | 675 | 14.338 | 10.292 | −4.013 | 1.00 | 18.96 |
| ATOM | 4566 | C | GLY | 675 | 13.602 | 11.384 | −4.770 | 1.00 | 19.21 |
| ATOM | 4567 | O | GLY | 675 | 13.982 | 11.737 | −5.890 | 1.00 | 17.51 |
| ATOM | 4568 | N | ALA | 676 | 12.548 | 11.916 | −4.160 | 1.00 | 19.23 |
| ATOM | 4569 | CA | ALA | 676 | 11.737 | 12.969 | −4.771 | 1.00 | 21.38 |
| ATOM | 4570 | CB | ALA | 676 | 10.515 | 12.347 | −5.445 | 1.00 | 22.27 |
| ATOM | 4571 | C | ALA | 676 | 11.291 | 14.003 | −3.731 | 1.00 | 21.87 |
| ATOM | 4572 | O | ALA | 676 | 10.160 | 13.970 | −3.260 | 1.00 | 21.80 |
| ATOM | 4573 | N | PRO | 677 | 12.174 | 14.951 | −3.376 | 1.00 | 23.81 |
| ATOM | 4574 | CD | PRO | 677 | 13.544 | 15.130 | −3.888 | 1.00 | 24.84 |
| ATOM | 4575 | CA | PRO | 677 | 11.854 | 15.984 | −2.384 | 1.00 | 24.40 |
| ATOM | 4576 | CB | PRO | 677 | 13.204 | 16.654 | −2.140 | 1.00 | 24.98 |
| ATOM | 4577 | CG | PRO | 677 | 13.859 | 16.554 | −3.459 | 1.00 | 25.58 |
| ATOM | 4578 | C | PRO | 677 | 10.774 | 16.989 | −2.762 | 1.00 | 23.70 |
| ATOM | 4579 | O | PRO | 677 | 10.197 | 17.640 | −1.891 | 1.00 | 25.65 |
| ATOM | 4580 | N | ASN | 678 | 10.496 | 17.114 | −4.054 | 1.00 | 24.73 |
| ATOM | 4581 | CA | ASN | 678 | 9.483 | 18.057 | −4.497 | 1.00 | 24.76 |
| ATOM | 4582 | CB | ASN | 678 | 9.994 | 18.852 | −5.706 | 1.00 | 27.05 |
| ATOM | 4583 | CG | ASN | 678 | 11.256 | 19.634 | −5.395 | 1.00 | 30.19 |
| ATOM | 4584 | OD1 | ASN | 678 | 11.303 | 20.400 | −4.430 | 1.00 | 31.87 |
| ATOM | 4585 | ND2 | ASN | 678 | 12.291 | 19.443 | −6.212 | 1.00 | 31.96 |
| ATOM | 4586 | C | ASN | 678 | 8.164 | 17.385 | −4.845 | 1.00 | 22.32 |
| ATOM | 4587 | O | ASN | 678 | 7.206 | 18.056 | −5.205 | 1.00 | 21.78 |
| ATOM | 4588 | N | CYS | 679 | 8.093 | 16.064 | −4.731 | 1.00 | 21.57 |
| ATOM | 4589 | CA | CYS | 679 | 6.845 | 15.393 | −5.080 | 1.00 | 20.82 |
| ATOM | 4590 | CB | CYS | 679 | 7.088 | 13.904 | −5.328 | 1.00 | 22.07 |
| ATOM | 4591 | SG | CYS | 679 | 6.942 | 12.839 | −3.863 | 1.00 | 21.95 |
| ATOM | 4592 | C | CYS | 679 | 5.761 | 15.549 | −4.017 | 1.00 | 20.00 |
| ATOM | 4593 | O | CYS | 679 | 6.045 | 15.896 | −2.871 | 1.00 | 20.40 |
| ATOM | 4594 | N | LEU | 680 | 4.513 | 15.336 | −4.432 | 1.00 | 19.40 |
| ATOM | 4595 | CA | LEU | 680 | 3.380 | 15.365 | −3.520 | 1.00 | 18.90 |
| ATOM | 4596 | CB | LEU | 680 | 2.074 | 15.699 | −4.242 | 1.00 | 17.17 |
| ATOM | 4597 | CG | LEU | 680 | 0.812 | 15.597 | −3.385 | 1.00 | 16.51 |
| ATOM | 4598 | CD1 | LEU | 680 | 0.927 | 16.514 | −2.164 | 1.00 | 17.54 |
| ATOM | 4599 | CD2 | LEU | 680 | −0.406 | 15.947 | −4.211 | 1.00 | 16.35 |
| ATOM | 4600 | C | LEU | 680 | 3.354 | 13.917 | −3.056 | 1.00 | 18.47 |
| ATOM | 4601 | O | LEU | 680 | 3.069 | 13.014 | −3.846 | 1.00 | 17.45 |
| ATOM | 4602 | N | LEU | 681 | 3.659 | 13.699 | −1.782 | 1.00 | 18.84 |
| ATOM | 4603 | CA | LEU | 681 | 3.734 | 12.346 | −1.245 | 1.00 | 18.21 |
| ATOM | 4604 | CB | LEU | 681 | 5.054 | 12.192 | −0.474 | 1.00 | 18.92 |
| ATOM | 4605 | CG | LEU | 681 | 5.611 | 10.804 | −0.137 | 1.00 | 20.05 |
| ATOM | 4606 | CD1 | LEU | 681 | 7.014 | 10.970 | 0.436 | 1.00 | 18.66 |
| ATOM | 4607 | CD2 | LEU | 681 | 4.714 | 10.091 | 0.868 | 1.00 | 21.30 |
| ATOM | 4608 | C | LEU | 681 | 2.566 | 11.966 | −0.353 | 1.00 | 16.59 |
| ATOM | 4609 | O | LEU | 681 | 2.356 | 12.562 | 0.701 | 1.00 | 16.26 |
| ATOM | 4610 | N | LEU | 682 | 1.802 | 10.974 | −0.790 | 1.00 | 16.90 |
| ATOM | 4611 | CA | LEU | 682 | 0.677 | 10.491 | −0.007 | 1.00 | 17.13 |
| ATOM | 4612 | CB | LEU | 682 | −0.520 | 10.151 | −0.900 | 1.00 | 18.79 |
| ATOM | 4613 | CG | LEU | 682 | −1.449 | 11.291 | −1.333 | 1.00 | 22.69 |
| ATOM | 4614 | CD1 | LEU | 682 | −0.700 | 12.278 | −2.227 | 1.00 | 23.30 |
| ATOM | 4615 | CD2 | LEU | 682 | −2.640 | 10.700 | −2.070 | 1.00 | 23.40 |
| ATOM | 4616 | C | LEU | 682 | 1.156 | 9.233 | 0.690 | 1.00 | 16.92 |
| ATOM | 4617 | O | LEU | 682 | 1.870 | 8.430 | 0.098 | 1.00 | 18.52 |
| ATOM | 4618 | N | ALA | 683 | 0.803 | 9.074 | 1.957 | 1.00 | 14.89 |
| ATOM | 4619 | CA | ALA | 683 | 1.195 | 7.869 | 2.682 | 1.00 | 14.34 |
| ATOM | 4620 | CB | ALA | 683 | 2.223 | 8.201 | 3.753 | 1.00 | 14.56 |
| ATOM | 4621 | C | ALA | 683 | −0.049 | 7.260 | 3.308 | 1.00 | 13.19 |
| ATOM | 4622 | O | ALA | 683 | −0.893 | 7.967 | 3.857 | 1.00 | 12.69 |
| ATOM | 4623 | N | ASP | 684 | −0.161 | 5.943 | 3.204 | 1.00 | 13.76 |
| ATOM | 4624 | CA | ASP | 684 | −1.292 | 5.224 | 3.761 | 1.00 | 13.79 |
| ATOM | 4625 | CB | ASP | 684 | −1.369 | 3.811 | 3.199 | 1.00 | 17.15 |
| ATOM | 4626 | CG | ASP | 684 | −2.237 | 3.701 | 1.972 | 1.00 | 18.93 |
| ATOM | 4627 | OD1 | ASP | 684 | −2.988 | 4.647 | 1.660 | 1.00 | 17.43 |
| ATOM | 4628 | OD2 | ASP | 684 | −2.174 | 2.630 | 1.334 | 1.00 | 19.86 |
| ATOM | 4629 | C | ASP | 684 | −1.192 | 5.050 | 5.256 | 1.00 | 14.01 |
| ATOM | 4630 | O | ASP | 684 | −0.098 | 4.854 | 5.791 | 1.00 | 13.68 |
| ATOM | 4631 | N | LEU | 685 | −2.329 | 5.126 | 5.938 | 1.00 | 14.34 |
| ATOM | 4632 | CA | LEU | 685 | −2.335 | 4.819 | 7.359 | 1.00 | 15.73 |
| ATOM | 4633 | CB | LEU | 685 | −3.430 | 5.568 | 8.128 | 1.00 | 17.73 |
| ATOM | 4634 | CG | LEU | 685 | −3.114 | 7.008 | 8.538 | 1.00 | 18.92 |
| ATOM | 4635 | CD1 | LEU | 685 | −4.058 | 7.437 | 9.658 | 1.00 | 20.70 |
| ATOM | 4636 | CD2 | LEU | 685 | −1.670 | 7.106 | 9.013 | 1.00 | 20.52 |
| ATOM | 4637 | C | LEU | 685 | −2.713 | 3.346 | 7.219 | 1.00 | 15.76 |
| ATOM | 4638 | O | LEU | 685 | −3.731 | 3.022 | 6.616 | 1.00 | 14.86 |
| ATOM | 4639 | N | PRO | 686 | −1.876 | 2.433 | 7.734 | 1.00 | 15.32 |
| ATOM | 4640 | CD | PRO | 686 | −0.579 | 2.697 | 8.380 | 1.00 | 16.43 |
| ATOM | 4641 | CA | PRO | 686 | −2.139 | 0.996 | 7.643 | 1.00 | 16.10 |
| ATOM | 4642 | CB | PRO | 686 | −0.809 | 0.380 | 8.085 | 1.00 | 17.06 |
| ATOM | 4643 | CG | PRO | 686 | −0.283 | 1.383 | 9.073 | 1.00 | 16.35 |
| ATOM | 4644 | C | PRO | 686 | −3.327 | 0.488 | 8.458 | 1.00 | 16.41 |
| ATOM | 4645 | O | PRO | 686 | −4.008 | 1.254 | 9.143 | 1.00 | 16.37 |
| ATOM | 4646 | N | PHE | 687 | −3.563 | −0.815 | 8.358 | 1.00 | 15.03 |
| ATOM | 4647 | CA | PHE | 687 | −4.647 | −1.494 | 9.062 | 1.00 | 15.40 |
| ATOM | 4648 | CB | PHE | 687 | −4.470 | −3.007 | 8.892 | 1.00 | 14.01 |
| ATOM | 4649 | CG | PHE | 687 | −5.265 | −3.841 | 9.861 | 1.00 | 15.81 |
| ATOM | 4650 | CD1 | PHE | 687 | −6.655 | −3.762 | 9.905 | 1.00 | 16.16 |
| ATOM | 4651 | CD2 | PHE | 687 | −4.619 | −4.758 | 10.685 | 1.00 | 14.41 |
| ATOM | 4652 | CE1 | PHE | 687 | −7.390 | −4.595 | 10.751 | 1.00 | 16.59 |
| ATOM | 4653 | CE2 | PHE | 687 | −5.337 | −5.597 | 11.535 | 1.00 | 14.28 |
| ATOM | 4654 | CZ | PHE | 687 | −6.726 | −5.516 | 11.568 | 1.00 | 18.27 |
| ATOM | 4655 | C | PHE | 687 | −4.663 | −1.106 | 10.538 | 1.00 | 13.34 |
| ATOM | 4656 | O | PHE | 687 | −3.634 | −1.136 | 11.209 | 1.00 | 12.84 |
| ATOM | 4657 | N | MET | 688 | −5.841 | −0.732 | 11.027 | 1.00 | 14.65 |
| ATOM | 4658 | CA | MET | 688 | −6.035 | −0.332 | 12.418 | 1.00 | 14.46 |
| ATOM | 4659 | CB | MET | 688 | −5.922 | −1.558 | 13.340 | 1.00 | 16.06 |
| ATOM | 4660 | CG | MET | 688 | −6.712 | −1.415 | 14.648 | 1.00 | 16.59 |
| ATOM | 4661 | SD | MET | 688 | −8.529 | −1.347 | 14.400 | 1.00 | 16.99 |
| ATOM | 4662 | CE | MET | 688 | −8.858 | −3.054 | 14.056 | 1.00 | 19.17 |
| ATOM | 4663 | C | MET | 688 | −5.087 | 0.767 | 12.911 | 1.00 | 14.94 |
| ATOM | 4664 | O | MET | 688 | −4.718 | 0.793 | 14.087 | 1.00 | 18.65 |
| ATOM | 4665 | N | ALA | 689 | −4.680 | 1.676 | 12.031 | 1.00 | 14.13 |
| ATOM | 4666 | CA | ALA | 689 | −3.803 | 2.765 | 12.466 | 1.00 | 13.66 |
| ATOM | 4667 | CB | ALA | 689 | −2.719 | 3.034 | 11.421 | 1.00 | 15.41 |
| ATOM | 4668 | C | ALA | 689 | −4.610 | 4.046 | 12.716 | 1.00 | 13.55 |
| ATOM | 4669 | O | ALA | 689 | −4.051 | 5.085 | 13.051 | 1.00 | 14.39 |
| ATOM | 4670 | N | TYR | 690 | −5.923 | 3.971 | 12.545 | 1.00 | 13.83 |
| ATOM | 4671 | CA | TYR | 690 | −6.775 | 5.133 | 12.763 | 1.00 | 13.76 |
| ATOM | 4672 | CB | TYR | 690 | −7.009 | 5.875 | 11.433 | 1.00 | 14.18 |
| ATOM | 4673 | CG | TYR | 690 | −7.351 | 4.979 | 10.261 | 1.00 | 12.90 |
| ATOM | 4674 | CD1 | TYR | 690 | −8.666 | 4.841 | 9.918 | 1.00 | 12.68 |
| ATOM | 4675 | CE1 | TYR | 690 | −8.981 | 4.004 | 8.741 | 1.00 | 13.27 |
| ATOM | 4676 | CD2 | TYR | 690 | −6.352 | 4.257 | 9.599 | 1.00 | 13.42 |
| ATOM | 4677 | CE2 | TYR | 690 | −6.651 | 3.418 | 8.526 | 1.00 | 14.00 |
| ATOM | 4678 | CZ | TYR | 690 | −7.963 | 3.297 | 8.101 | 1.00 | 13.04 |
| ATOM | 4679 | OH | TYR | 690 | −8.246 | 2.468 | 7.030 | 1.00 | 14.87 |
| ATOM | 4680 | C | TYR | 690 | −8.087 | 4.693 | 13.392 | 1.00 | 14.99 |
| ATOM | 4681 | O | TYR | 690 | −9.166 | 5.144 | 13.005 | 1.00 | 15.19 |
| ATOM | 4682 | N | ALA | 691 | −7.975 | 3.809 | 14.382 | 1.00 | 13.50 |
| ATOM | 4683 | CA | ALA | 691 | −9.135 | 3.239 | 15.082 | 1.00 | 14.83 |
| ATOM | 4684 | CB | ALA | 691 | −8.673 | 2.102 | 15.993 | 1.00 | 13.88 |
| ATOM | 4685 | C | ALA | 691 | −9.924 | 4.257 | 15.888 | 1.00 | 13.52 |
| ATOM | 4686 | O | ALA | 691 | −11.120 | 4.084 | 16.110 | 1.00 | 14.21 |
| ATOM | 4687 | N | THR | 692 | −9.235 | 5.293 | 16.354 | 1.00 | 15.07 |
| ATOM | 4688 | CA | THR | 692 | −9.856 | 6.374 | 17.109 | 1.00 | 16.94 |
| ATOM | 4689 | CB | THR | 692 | −9.608 | 6.268 | 18.629 | 1.00 | 14.55 |
| ATOM | 4690 | OG1 | THR | 692 | −8.219 | 6.492 | 18.902 | 1.00 | 15.54 |
| ATOM | 4691 | CG2 | THR | 692 | −10.015 | 4.897 | 19.152 | 1.00 | 15.71 |
| ATOM | 4692 | C | THR | 692 | −9.165 | 7.635 | 16.636 | 1.00 | 18.24 |
| ATOM | 4693 | O | THR | 692 | −8.080 | 7.576 | 16.059 | 1.00 | 18.19 |
| ATOM | 4694 | N | PRO | 693 | −9.790 | 8.794 | 16.864 | 1.00 | 17.17 |
| ATOM | 4695 | CD | PRO | 693 | −11.169 | 8.968 | 17.351 | 1.00 | 18.47 |
| ATOM | 4696 | CA | PRO | 693 | −9.209 | 10.074 | 16.457 | 1.00 | 18.57 |
| ATOM | 4697 | CB | PRO | 693 | −10.195 | 11.090 | 17.032 | 1.00 | 20.23 |
| ATOM | 4698 | CG | PRO | 693 | −11.512 | 10.368 | 16.873 | 1.00 | 19.28 |
| ATOM | 4699 | C | PRO | 693 | −7.801 | 10.241 | 17.021 | 1.00 | 18.65 |
| ATOM | 4700 | O | PRO | 693 | −6.853 | 10.516 | 16.283 | 1.00 | 15.76 |
| ATOM | 4701 | N | GLU | 694 | −7.668 | 10.064 | 18.334 | 1.00 | 18.32 |
| ATOM | 4702 | CA | GLU | 694 | −6.371 | 10.194 | 18.987 | 1.00 | 21.05 |
| ATOM | 4703 | CB | GLU | 694 | −6.485 | 9.871 | 20.486 | 1.00 | 24.65 |
| ATOM | 4704 | CG | GLU | 694 | −6.958 | 11.039 | 21.341 | 1.00 | 32.62 |
| ATOM | 4705 | CD | GLU | 694 | −6.747 | 10.804 | 22.832 | 1.00 | 35.61 |
| ATOM | 4706 | OE1 | GLU | 694 | −5.624 | 10.413 | 23.221 | 1.00 | 40.42 |
| ATOM | 4707 | OE2 | GLU | 694 | −7.694 | 11.024 | 23.616 | 1.00 | 40.47 |
| ATOM | 4708 | C | GLU | 694 | −5.290 | 9.321 | 18.352 | 1.00 | 18.88 |
| ATOM | 4709 | O | GLU | 694 | −4.156 | 9.766 | 18.181 | 1.00 | 19.69 |
| ATOM | 4710 | N | GLN | 695 | −5.621 | 8.078 | 18.013 | 1.00 | 18.90 |
| ATOM | 4711 | CA | GLN | 695 | −4.623 | 7.207 | 17.383 | 1.00 | 17.55 |
| ATOM | 4712 | CB | GLN | 695 | −5.095 | 5.759 | 17.375 | 1.00 | 19.52 |
| ATOM | 4713 | CG | GLN | 695 | −5.109 | 5.143 | 18.762 | 1.00 | 25.76 |
| ATOM | 4714 | CD | GLN | 695 | −5.606 | 3.722 | 18.754 | 1.00 | 26.70 |
| ATOM | 4715 | OE1 | GLN | 695 | −5.007 | 2.849 | 18.128 | 1.00 | 28.17 |
| ATOM | 4716 | NE2 | GLN | 695 | −6.709 | 3.477 | 19.455 | 1.00 | 28.69 |
| ATOM | 4717 | C | GLN | 695 | −4.332 | 7.671 | 15.961 | 1.00 | 14.39 |
| ATOM | 4718 | O | GLN | 695 | −3.197 | 7.598 | 15.498 | 1.00 | 14.23 |
| ATOM | 4719 | N | ALA | 696 | −5.365 | 8.134 | 15.269 | 1.00 | 13.25 |
| ATOM | 4720 | CA | ALA | 696 | −5.187 | 8.637 | 13.913 | 1.00 | 12.91 |
| ATOM | 4721 | CB | ALA | 696 | −6.517 | 9.128 | 13.352 | 1.00 | 12.84 |
| ATOM | 4722 | C | ALA | 696 | −4.184 | 9.786 | 13.965 | 1.00 | 13.88 |
| ATOM | 4723 | O | ALA | 696 | −3.254 | 9.849 | 13.158 | 1.00 | 14.82 |
| ATOM | 4724 | N | PHE | 697 | −4.371 | 10.691 | 14.924 | 1.00 | 13.95 |
| ATOM | 4725 | CA | PHE | 697 | −3.478 | 11.849 | 15.071 | 1.00 | 14.74 |
| ATOM | 4726 | CB | PHE | 697 | −3.847 | 12.688 | 16.307 | 1.00 | 14.39 |
| ATOM | 4727 | CG | PHE | 697 | −5.272 | 13.165 | 16.326 | 1.00 | 14.70 |
| ATOM | 4728 | CD1 | PHE | 697 | −5.973 | 13.364 | 15.138 | 1.00 | 15.78 |
| ATOM | 4729 | CD2 | PHE | 697 | −5.913 | 13.417 | 17.535 | 1.00 | 17.31 |
| ATOM | 4730 | CE1 | PHE | 697 | −7.296 | 13.803 | 15.157 | 1.00 | 17.92 |
| ATOM | 4731 | CE2 | PHE | 697 | −7.235 | 13.858 | 17.567 | 1.00 | 18.65 |
| ATOM | 4732 | CZ | PHE | 697 | −7.928 | 14.050 | 16.373 | 1.00 | 17.31 |
| ATOM | 4733 | C | PHE | 697 | −2.015 | 11.456 | 15.206 | 1.00 | 16.50 |
| ATOM | 4734 | O | PHE | 697 | −1.155 | 11.972 | 14.491 | 1.00 | 13.69 |
| ATOM | 4735 | N | GLU | 698 | −1.747 | 10.543 | 16.138 | 1.00 | 15.66 |
| ATOM | 4736 | CA | GLU | 698 | −0.391 | 10.082 | 16.418 | 1.00 | 17.86 |
| ATOM | 4737 | CB | GLU | 698 | −0.409 | 9.111 | 17.603 | 1.00 | 21.29 |
| ATOM | 4738 | CG | GLU | 698 | 0.971 | 8.622 | 18.050 | 1.00 | 30.05 |
| ATOM | 4739 | CD | GLU | 698 | 1.631 | 9.547 | 19.063 | 1.00 | 34.72 |
| ATOM | 4740 | OE1 | GLU | 698 | 1.784 | 10.753 | 18.766 | 1.00 | 37.42 |
| ATOM | 4741 | OE2 | GLU | 698 | 1.998 | 9.064 | 20.165 | 1.00 | 40.64 |
| ATOM | 4742 | C | GLU | 698 | 0.248 | 9.399 | 15.213 | 1.00 | 16.35 |
| ATOM | 4743 | O | GLU | 698 | 1.385 | 9.702 | 14.836 | 1.00 | 16.77 |
| ATOM | 4744 | N | ASN | 699 | −0.486 | 8.473 | 14.614 | 1.00 | 15.59 |
| ATOM | 4745 | CA | ASN | 699 | 0.033 | 7.743 | 13.471 | 1.00 | 15.22 |
| ATOM | 4746 | CB | ASN | 699 | −0.838 | 6.506 | 13.200 | 1.00 | 14.54 |
| ATOM | 4747 | CG | ASN | 699 | −0.748 | 5.488 | 14.328 | 1.00 | 17.82 |
| ATOM | 4748 | OD1 | ASN | 699 | 0.320 | 5.316 | 14.927 | 1.00 | 15.96 |
| ATOM | 4749 | ND2 | ASN | 699 | −1.857 | 4.809 | 14.621 | 1.00 | 15.38 |
| ATOM | 4750 | C | ASN | 699 | 0.159 | 8.636 | 12.236 | 1.00 | 14.83 |
| ATOM | 4751 | O | ASN | 699 | 1.134 | 8.535 | 11.488 | 1.00 | 14.23 |
| ATOM | 4752 | N | ALA | 700 | −0.802 | 9.529 | 12.026 | 1.00 | 14.50 |
| ATOM | 4753 | CA | ALA | 700 | −0.710 | 10.431 | 10.880 | 1.00 | 15.69 |
| ATOM | 4754 | CB | ALA | 700 | −1.961 | 11.288 | 10.781 | 1.00 | 14.56 |
| ATOM | 4755 | C | ALA | 700 | 0.525 | 11.316 | 11.044 | 1.00 | 15.36 |
| ATOM | 4756 | O | ALA | 700 | 1.253 | 11.555 | 10.089 | 1.00 | 14.90 |
| ATOM | 4757 | N | ALA | 701 | 0.770 | 11.793 | 12.262 | 1.00 | 15.92 |
| ATOM | 4758 | CA | ALA | 701 | 1.920 | 12.659 | 12.499 | 1.00 | 16.05 |
| ATOM | 4759 | CB | ALA | 701 | 1.888 | 13.201 | 13.920 | 1.00 | 15.03 |
| ATOM | 4760 | C | ALA | 701 | 3.232 | 11.930 | 12.244 | 1.00 | 15.03 |
| ATOM | 4761 | O | ALA | 701 | 4.184 | 12.509 | 11.730 | 1.00 | 16.35 |
| ATOM | 4762 | N | THR | 702 | 3.289 | 10.657 | 12.612 | 1.00 | 14.38 |
| ATOM | 4763 | CA | THR | 702 | 4.503 | 9.878 | 12.411 | 1.00 | 12.85 |
| ATOM | 4764 | CB | THR | 702 | 4.323 | 8.450 | 12.956 | 1.00 | 12.19 |
| ATOM | 4765 | OG1 | THR | 702 | 4.034 | 8.522 | 14.354 | 1.00 | 12.71 |
| ATOM | 4766 | CG2 | THR | 702 | 5.577 | 7.625 | 12.744 | 1.00 | 12.48 |
| ATOM | 4767 | C | THR | 702 | 4.861 | 9.817 | 10.928 | 1.00 | 15.73 |
| ATOM | 4768 | O | THR | 702 | 6.015 | 9.991 | 10.549 | 1.00 | 14.96 |
| ATOM | 4769 | N | VAL | 703 | 3.855 | 9.594 | 10.095 | 1.00 | 14.32 |
| ATOM | 4770 | CA | VAL | 703 | 4.061 | 9.494 | 8.665 | 1.00 | 17.24 |
| ATOM | 4771 | CB | VAL | 703 | 2.825 | 8.862 | 8.019 | 1.00 | 18.94 |
| ATOM | 4772 | CG1 | VAL | 703 | 2.763 | 9.181 | 6.557 | 1.00 | 24.70 |
| ATOM | 4773 | CG2 | VAL | 703 | 2.866 | 7.360 | 8.248 | 1.00 | 17.69 |
| ATOM | 4774 | C | VAL | 703 | 4.373 | 10.850 | 8.037 | 1.00 | 17.44 |
| ATOM | 4775 | O | VAL | 703 | 5.207 | 10.944 | 7.131 | 1.00 | 17.66 |
| ATOM | 4776 | N | MET | 704 | 3.705 | 11.895 | 8.519 | 1.00 | 15.75 |
| ATOM | 4777 | CA | MET | 704 | 3.942 | 13.246 | 8.020 | 1.00 | 18.71 |
| ATOM | 4778 | CB | MET | 704 | 2.926 | 14.231 | 8.615 | 1.00 | 19.63 |
| ATOM | 4779 | CG | MET | 704 | 1.461 | 13.959 | 8.263 | 1.00 | 24.43 |
| ATOM | 4780 | SD | MET | 704 | 1.046 | 14.259 | 6.517 | 1.00 | 27.09 |
| ATOM | 4781 | CE | MET | 704 | 1.225 | 16.072 | 6.424 | 1.00 | 25.27 |
| ATOM | 4782 | C | MET | 704 | 5.373 | 13.691 | 8.379 | 1.00 | 17.56 |
| ATOM | 4783 | O | MET | 704 | 6.075 | 14.240 | 7.534 | 1.00 | 15.47 |
| ATOM | 4784 | N | ARG | 705 | 5.810 | 13.459 | 9.619 | 1.00 | 16.57 |
| ATOM | 4785 | CA | ARG | 705 | 7.160 | 13.856 | 10.007 | 1.00 | 17.21 |
| ATOM | 4786 | CB | ARG | 705 | 7.424 | 13.597 | 11.499 | 1.00 | 18.12 |
| ATOM | 4787 | CG | ARG | 705 | 6.566 | 14.413 | 12.470 | 1.00 | 17.42 |
| ATOM | 4788 | CD | ARG | 705 | 7.139 | 14.379 | 13.887 | 1.00 | 18.37 |
| ATOM | 4789 | NE | ARG | 705 | 6.188 | 14.923 | 14.862 | 1.00 | 21.69 |
| ATOM | 4790 | CZ | ARG | 705 | 5.169 | 14.242 | 15.377 | 1.00 | 21.26 |
| ATOM | 4791 | NH1 | ARG | 705 | 4.960 | 12.980 | 15.024 | 1.00 | 23.11 |
| ATOM | 4792 | NH2 | ARG | 705 | 4.340 | 14.830 | 16.225 | 1.00 | 22.67 |
| ATOM | 4793 | C | ARG | 705 | 8.196 | 13.104 | 9.193 | 1.00 | 17.07 |
| ATOM | 4794 | O | ARG | 705 | 9.330 | 13.568 | 9.027 | 1.00 | 17.74 |
| ATOM | 4795 | N | ALA | 706 | 7.807 | 11.942 | 8.682 | 1.00 | 16.11 |
| ATOM | 4796 | CA | ALA | 706 | 8.709 | 11.112 | 7.891 | 1.00 | 15.58 |
| ATOM | 4797 | CB | ALA | 706 | 8.235 | 9.662 | 7.921 | 1.00 | 14.95 |
| ATOM | 4798 | C | ALA | 706 | 8.847 | 11.584 | 6.445 | 1.00 | 15.19 |
| ATOM | 4799 | O | ALA | 706 | 9.688 | 11.068 | 5.709 | 1.00 | 15.93 |
| ATOM | 4800 | N | GLY | 707 | 8.022 | 12.544 | 6.033 | 1.00 | 14.94 |
| ATOM | 4801 | CA | GLY | 707 | 8.130 | 13.053 | 4.677 | 1.00 | 15.89 |
| ATOM | 4802 | C | GLY | 707 | 6.853 | 13.203 | 3.871 | 1.00 | 16.03 |
| ATOM | 4803 | O | GLY | 707 | 6.851 | 13.882 | 2.843 | 1.00 | 17.90 |
| ATOM | 4804 | N | ALA | 708 | 5.764 | 12.591 | 4.321 | 1.00 | 15.28 |
| ATOM | 4805 | CA | ALA | 708 | 4.500 | 12.675 | 3.598 | 1.00 | 14.65 |
| ATOM | 4806 | CB | ALA | 708 | 3.524 | 11.646 | 4.140 | 1.00 | 11.62 |
| ATOM | 4807 | C | ALA | 708 | 3.870 | 14.060 | 3.677 | 1.00 | 15.64 |
| ATOM | 4808 | O | ALA | 708 | 4.102 | 14.809 | 4.628 | 1.00 | 14.42 |
| ATOM | 4809 | N | ASN | 709 | 3.061 | 14.394 | 2.675 | 1.00 | 16.20 |
| ATOM | 4810 | CA | ASN | 709 | 2.369 | 15.682 | 2.660 | 1.00 | 17.11 |
| ATOM | 4811 | CB | ASN | 709 | 2.482 | 16.363 | 1.297 | 1.00 | 16.35 |
| ATOM | 4812 | CG | ASN | 709 | 3.905 | 16.548 | 0.855 | 1.00 | 16.63 |
| ATOM | 4813 | OD1 | ASN | 709 | 4.693 | 17.223 | 1.518 | 1.00 | 19.11 |
| ATOM | 4814 | ND2 | ASN | 709 | 4.246 | 15.951 | −0.271 | 1.00 | 13.26 |
| ATOM | 4815 | C | ASN | 709 | 0.890 | 15.455 | 2.931 | 1.00 | 17.83 |
| ATOM | 4816 | O | ASN | 709 | 0.163 | 16.388 | 3.252 | 1.00 | 18.60 |
| ATOM | 4817 | N | MET | 710 | 0.448 | 14.212 | 2.779 | 1.00 | 17.20 |
| ATOM | 4818 | CA | MET | 710 | −0.955 | 13.871 | 2.969 | 1.00 | 16.87 |
| ATOM | 4819 | CB | MET | 710 | −1.713 | 14.127 | 1.654 | 1.00 | 18.60 |
| ATOM | 4820 | CG | MET | 710 | −3.200 | 13.816 | 1.659 | 1.00 | 20.01 |
| ATOM | 4821 | SD | MET | 710 | −3.944 | 14.052 | −0.001 | 1.00 | 21.60 |
| ATOM | 4822 | CE | MET | 710 | −4.743 | 15.673 | 0.168 | 1.00 | 23.23 |
| ATOM | 4823 | C | MET | 710 | −1.065 | 12.405 | 3.377 | 1.00 | 16.40 |
| ATOM | 4824 | O | MET | 710 | −0.177 | 11.604 | 3.085 | 1.00 | 14.55 |
| ATOM | 4825 | N | VAL | 711 | −2.152 | 12.070 | 4.065 | 1.00 | 16.03 |
| ATOM | 4826 | CA | VAL | 711 | −2.391 | 10.712 | 4.528 | 1.00 | 16.57 |
| ATOM | 4827 | CB | VAL | 711 | −2.552 | 10.697 | 6.065 | 1.00 | 19.08 |
| ATOM | 4828 | CG1 | VAL | 711 | −3.021 | 9.352 | 6.515 | 1.00 | 23.70 |
| ATOM | 4829 | CG2 | VAL | 711 | −1.230 | 11.054 | 6.730 | 1.00 | 17.88 |
| ATOM | 4830 | C | VAL | 711 | −3.655 | 10.132 | 3.896 | 1.00 | 15.86 |
| ATOM | 4831 | O | VAL | 711 | −4.635 | 10.851 | 3.690 | 1.00 | 14.07 |
| ATOM | 4832 | N | LYS | 712 | −3.631 | 8.840 | 3.570 | 1.00 | 13.84 |
| ATOM | 4833 | CA | LYS | 712 | −4.798 | 8.187 | 2.985 | 1.00 | 14.28 |
| ATOM | 4834 | CB | LYS | 712 | −4.445 | 7.501 | 1.648 | 1.00 | 14.48 |
| ATOM | 4835 | CG | LYS | 712 | −5.645 | 6.773 | 1.006 | 1.00 | 13.64 |
| ATOM | 4836 | CD | LYS | 712 | −5.475 | 6.476 | −0.491 | 1.00 | 14.90 |
| ATOM | 4837 | CE | LYS | 712 | −4.471 | 5.361 | −0.770 | 1.00 | 14.70 |
| ATOM | 4838 | NZ | LYS | 712 | −4.882 | 4.059 | −0.157 | 1.00 | 15.92 |
| ATOM | 4839 | C | LYS | 712 | −5.371 | 7.160 | 3.960 | 1.00 | 15.39 |
| ATOM | 4840 | O | LYS | 712 | −4.632 | 6.361 | 4.532 | 1.00 | 13.66 |
| ATOM | 4841 | N | ILE | 713 | −6.683 | 7.203 | 4.167 | 1.00 | 15.54 |
| ATOM | 4842 | CA | ILE | 713 | −7.349 | 6.260 | 5.060 | 1.00 | 16.63 |
| ATOM | 4843 | CB | ILE | 713 | −7.800 | 6.949 | 6.379 | 1.00 | 18.02 |
| ATOM | 4844 | CG2 | ILE | 713 | −6.584 | 7.372 | 7.181 | 1.00 | 19.14 |
| ATOM | 4845 | CG1 | ILE | 713 | −8.667 | 8.173 | 6.072 | 1.00 | 20.03 |
| ATOM | 4846 | CD1 | ILE | 713 | −9.130 | 8.925 | 7.332 | 1.00 | 19.76 |
| ATOM | 4847 | C | ILE | 713 | −8.553 | 5.642 | 4.351 | 1.00 | 18.46 |
| ATOM | 4848 | O | ILE | 713 | −9.224 | 6.306 | 3.561 | 1.00 | 17.02 |
| ATOM | 4849 | N | GLU | 714 | −8.809 | 4.363 | 4.622 | 1.00 | 17.38 |
| ATOM | 4850 | CA | GLU | 714 | −9.917 | 3.642 | 3.995 | 1.00 | 18.87 |
| ATOM | 4851 | CB | GLU | 714 | −9.530 | 2.180 | 3.735 | 1.00 | 18.90 |
| ATOM | 4852 | CG | GLU | 714 | −8.183 | 1.990 | 3.074 | 1.00 | 22.06 |
| ATOM | 4853 | CD | GLU | 714 | −7.866 | 0.530 | 2.797 | 1.00 | 23.62 |
| ATOM | 4854 | OE1 | GLU | 714 | −8.597 | −0.357 | 3.292 | 1.00 | 22.57 |
| ATOM | 4855 | OE2 | GLU | 714 | −6.876 | 0.267 | 2.082 | 1.00 | 26.90 |
| ATOM | 4856 | C | GLU | 714 | −11.162 | 3.649 | 4.867 | 1.00 | 18.79 |
| ATOM | 4857 | O | GLU | 714 | −11.091 | 3.389 | 6.069 | 1.00 | 17.88 |
| ATOM | 4858 | N | GLY | 715 | −12.304 | 3.940 | 4.256 | 1.00 | 18.83 |
| ATOM | 4859 | CA | GLY | 715 | −13.545 | 3.956 | 5.002 | 1.00 | 20.89 |
| ATOM | 4860 | C | GLY | 715 | −14.476 | 5.056 | 4.553 | 1.00 | 21.05 |
| ATOM | 4861 | O | GLY | 715 | −14.063 | 5.980 | 3.855 | 1.00 | 22.49 |
| ATOM | 4862 | N | GLY | 716 | −15.738 | 4.953 | 4.958 | 1.00 | 21.69 |
| ATOM | 4863 | CA | GLY | 716 | −16.723 | 5.954 | 4.585 | 1.00 | 20.54 |
| ATOM | 4864 | C | GLY | 716 | −17.198 | 6.833 | 5.731 | 1.00 | 19.68 |
| ATOM | 4865 | O | GLY | 716 | −16.396 | 7.349 | 6.503 | 1.00 | 18.63 |
| ATOM | 4866 | N | GLU | 717 | −18.513 | 7.004 | 5.828 | 1.00 | 20.60 |
| ATOM | 4867 | CA | GLU | 717 | −19.143 | 7.828 | 6.857 | 1.00 | 23.06 |
| ATOM | 4868 | CB | GLU | 717 | −20.631 | 7.498 | 6.948 | 1.00 | 26.89 |
| ATOM | 4869 | CG | GLU | 717 | −21.512 | 8.436 | 6.154 | 1.00 | 35.80 |
| ATOM | 4870 | CD | GLU | 717 | −21.970 | 9.634 | 6.959 | 1.00 | 37.28 |
| ATOM | 4871 | OE1 | GLU | 717 | −21.125 | 10.277 | 7.616 | 1.00 | 39.12 |
| ATOM | 4872 | OE2 | GLU | 717 | −23.183 | 9.932 | 6.925 | 1.00 | 39.31 |
| ATOM | 4873 | C | GLU | 717 | −18.562 | 7.785 | 8.257 | 1.00 | 21.97 |
| ATOM | 4874 | O | GLU | 717 | −18.327 | 8.830 | 8.865 | 1.00 | 20.61 |
| ATOM | 4875 | N | TRP | 718 | −18.340 | 6.587 | 8.788 | 1.00 | 20.43 |
| ATOM | 4876 | CA | TRP | 718 | −17.813 | 6.487 | 10.145 | 1.00 | 18.97 |
| ATOM | 4877 | CB | TRP | 718 | −17.553 | 5.019 | 10.527 | 1.00 | 16.93 |
| ATOM | 4878 | CG | TRP | 718 | −16.338 | 4.387 | 9.900 | 1.00 | 17.31 |
| ATOM | 4879 | CD2 | TRP | 718 | −15.033 | 4.287 | 10.481 | 1.00 | 15.20 |
| ATOM | 4880 | CE2 | TRP | 718 | −14.213 | 3.604 | 9.554 | 1.00 | 17.66 |
| ATOM | 4881 | CE3 | TRP | 718 | −14.475 | 4.707 | 11.698 | 1.00 | 16.72 |
| ATOM | 4882 | CD1 | TRP | 718 | −16.260 | 3.780 | 8.677 | 1.00 | 16.47 |
| ATOM | 4883 | NE1 | TRP | 718 | −14.986 | 3.305 | 8.462 | 1.00 | 17.35 |
| ATOM | 4884 | CZ2 | TRP | 718 | −12.863 | 3.332 | 9.804 | 1.00 | 17.75 |
| ATOM | 4885 | CZ3 | TRP | 718 | −13.133 | 4.436 | 11.947 | 1.00 | 18.17 |
| ATOM | 4886 | CH2 | TRP | 718 | −12.342 | 3.753 | 11.001 | 1.00 | 18.73 |
| ATOM | 4887 | C | TRP | 718 | −16.554 | 7.318 | 10.372 | 1.00 | 17.36 |
| ATOM | 4888 | O | TRP | 718 | −16.263 | 7.697 | 11.504 | 1.00 | 16.96 |
| ATOM | 4889 | N | LEU | 719 | −15.829 | 7.624 | 9.295 | 1.00 | 17.61 |
| ATOM | 4890 | CA | LEU | 719 | −14.587 | 8.398 | 9.392 | 1.00 | 16.01 |
| ATOM | 4891 | CB | LEU | 719 | −13.634 | 7.998 | 8.264 | 1.00 | 19.58 |
| ATOM | 4892 | CG | LEU | 719 | −12.805 | 6.729 | 8.475 | 1.00 | 20.53 |
| ATOM | 4893 | CD1 | LEU | 719 | −12.035 | 6.426 | 7.205 | 1.00 | 20.90 |
| ATOM | 4894 | CD2 | LEU | 719 | −11.869 | 6.919 | 9.657 | 1.00 | 21.41 |
| ATOM | 4895 | C | LEU | 719 | −14.722 | 9.918 | 9.385 | 1.00 | 16.42 |
| ATOM | 4896 | O | LEU | 719 | −13.726 | 10.625 | 9.538 | 1.00 | 15.51 |
| ATOM | 4897 | N | VAL | 720 | −15.938 | 10.425 | 9.225 | 1.00 | 15.73 |
| ATOM | 4898 | CA | VAL | 720 | −16.154 | 11.869 | 9.181 | 1.00 | 16.54 |
| ATOM | 4899 | CB | VAL | 720 | −17.664 | 12.201 | 9.174 | 1.00 | 16.38 |
| ATOM | 4900 | CG1 | VAL | 720 | −17.875 | 13.699 | 9.349 | 1.00 | 16.79 |
| ATOM | 4901 | CG2 | VAL | 720 | −18.283 | 11.734 | 7.867 | 1.00 | 18.53 |
| ATOM | 4902 | C | VAL | 720 | −15.478 | 12.644 | 10.306 | 1.00 | 15.87 |
| ATOM | 4903 | O | VAL | 720 | −14.713 | 13.572 | 10.045 | 1.00 | 15.80 |
| ATOM | 4904 | N | GLU | 721 | −15.745 | 12.268 | 11.552 | 1.00 | 15.71 |
| ATOM | 4905 | CA | GLU | 721 | −15.133 | 12.966 | 12.680 | 1.00 | 16.66 |
| ATOM | 4906 | CB | GLU | 721 | −15.589 | 12.346 | 14.003 | 1.00 | 18.48 |
| ATOM | 4907 | CG | GLU | 721 | −14.947 | 12.976 | 15.227 | 1.00 | 22.68 |
| ATOM | 4908 | CD | GLU | 721 | −15.367 | 12.293 | 16.516 | 1.00 | 25.74 |
| ATOM | 4909 | OE1 | GLU | 721 | −14.966 | 11.131 | 16.732 | 1.00 | 27.93 |
| ATOM | 4910 | OE2 | GLU | 721 | −16.107 | 12.920 | 17.303 | 1.00 | 27.98 |
| ATOM | 4911 | C | GLU | 721 | −13.610 | 12.935 | 12.606 | 1.00 | 15.34 |
| ATOM | 4912 | O | GLU | 721 | −12.951 | 13.942 | 12.857 | 1.00 | 14.72 |
| ATOM | 4913 | N | THR | 722 | −13.055 | 11.772 | 12.274 | 1.00 | 14.80 |
| ATOM | 4914 | CA | THR | 722 | −11.608 | 11.624 | 12.175 | 1.00 | 14.47 |
| ATOM | 4915 | CB | THR | 722 | −11.213 | 10.164 | 11.860 | 1.00 | 16.94 |
| ATOM | 4916 | OG1 | THR | 722 | −11.580 | 9.322 | 12.964 | 1.00 | 19.05 |
| ATOM | 4917 | CG2 | THR | 722 | −9.710 | 10.057 | 11.628 | 1.00 | 15.42 |
| ATOM | 4918 | C | THR | 722 | −11.034 | 12.536 | 11.099 | 1.00 | 15.01 |
| ATOM | 4919 | O | THR | 722 | −10.001 | 13.167 | 11.299 | 1.00 | 14.67 |
| ATOM | 4920 | N | VAL | 723 | −11.703 | 12.606 | 9.954 | 1.00 | 16.05 |
| ATOM | 4921 | CA | VAL | 723 | −11.233 | 13.466 | 8.867 | 1.00 | 15.71 |
| ATOM | 4922 | CB | VAL | 723 | −12.058 | 13.236 | 7.587 | 1.00 | 15.17 |
| ATOM | 4923 | CG1 | VAL | 723 | −11.612 | 14.206 | 6.486 | 1.00 | 14.07 |
| ATOM | 4924 | CG2 | VAL | 723 | −11.880 | 11.807 | 7.122 | 1.00 | 15.82 |
| ATOM | 4925 | C | VAL | 723 | −11.307 | 14.943 | 9.268 | 1.00 | 16.42 |
| ATOM | 4926 | O | VAL | 723 | −10.353 | 15.694 | 9.079 | 1.00 | 16.04 |
| ATOM | 4927 | N | GLN | 724 | −12.437 | 15.354 | 9.831 | 1.00 | 18.63 |
| ATOM | 4928 | CA | GLN | 724 | −12.599 | 16.743 | 10.241 | 1.00 | 19.28 |
| ATOM | 4929 | CB | GLN | 724 | −13.980 | 16.955 | 10.869 | 1.00 | 22.80 |
| ATOM | 4930 | CG | GLN | 724 | −15.145 | 16.539 | 9.983 | 1.00 | 27.70 |
| ATOM | 4931 | CD | GLN | 724 | −16.488 | 16.695 | 10.675 | 1.00 | 30.60 |
| ATOM | 4932 | OE1 | GLN | 724 | −16.669 | 16.241 | 11.806 | 1.00 | 32.97 |
| ATOM | 4933 | NE2 | GLN | 724 | −17.440 | 17.328 | 9.996 | 1.00 | 32.67 |
| ATOM | 4934 | C | GLN | 724 | −11.513 | 17.132 | 11.241 | 1.00 | 19.06 |
| ATOM | 4935 | O | GLN | 724 | −10.880 | 18.180 | 11.107 | 1.00 | 16.76 |
| ATOM | 4936 | N | MET | 725 | −11.288 | 16.283 | 12.239 | 1.00 | 15.76 |
| ATOM | 4937 | CA | MET | 725 | −10.280 | 16.576 | 13.254 | 1.00 | 15.65 |
| ATOM | 4938 | CB | MET | 725 | −10.440 | 15.634 | 14.445 | 1.00 | 14.12 |
| ATOM | 4939 | CG | MET | 725 | −11.733 | 15.858 | 15.188 | 1.00 | 17.99 |
| ATOM | 4940 | SD | MET | 725 | −11.866 | 14.852 | 16.672 | 1.00 | 21.90 |
| ATOM | 4941 | CE | MET | 725 | −10.847 | 15.827 | 17.760 | 1.00 | 19.31 |
| ATOM | 4942 | C | MET | 725 | −8.849 | 16.537 | 12.744 | 1.00 | 14.34 |
| ATOM | 4943 | O | MET | 725 | −8.018 | 17.336 | 13.171 | 1.00 | 14.82 |
| ATOM | 4944 | N | LEU | 726 | −8.553 | 15.615 | 11.834 | 1.00 | 13.91 |
| ATOM | 4945 | CA | LEU | 726 | −7.207 | 15.540 | 11.292 | 1.00 | 15.00 |
| ATOM | 4946 | CB | LEU | 726 | −7.067 | 14.333 | 10.366 | 1.00 | 13.52 |
| ATOM | 4947 | CG | LEU | 726 | −6.658 | 13.016 | 11.034 | 1.00 | 15.57 |
| ATOM | 4948 | CD1 | LEU | 726 | −6.916 | 11.860 | 10.087 | 1.00 | 15.92 |
| ATOM | 4949 | CD2 | LEU | 726 | −5.205 | 13.077 | 11.443 | 1.00 | 15.18 |
| ATOM | 4950 | C | LEU | 726 | −6.873 | 16.819 | 10.526 | 1.00 | 16.85 |
| ATOM | 4951 | O | LEU | 726 | −5.783 | 17.376 | 10.664 | 1.00 | 16.74 |
| ATOM | 4952 | N | THR | 727 | −7.822 | 17.275 | 9.716 | 1.00 | 17.73 |
| ATOM | 4953 | CA | THR | 727 | −7.640 | 18.482 | 8.909 | 1.00 | 22.25 |
| ATOM | 4954 | CB | THR | 727 | −8.885 | 18.755 | 8.050 | 1.00 | 23.64 |
| ATOM | 4955 | OG1 | THR | 727 | −9.135 | 17.618 | 7.207 | 1.00 | 25.71 |
| ATOM | 4956 | CG2 | THR | 727 | −8.669 | 19.989 | 7.171 | 1.00 | 26.68 |
| ATOM | 4957 | C | THR | 727 | −7.332 | 19.719 | 9.747 | 1.00 | 22.81 |
| ATOM | 4958 | O | THR | 727 | −6.363 | 20.442 | 9.473 | 1.00 | 22.47 |
| ATOM | 4959 | N | GLU | 728 | −8.143 | 19.968 | 10.769 | 1.00 | 23.34 |
| ATOM | 4960 | CA | GLU | 728 | −7.903 | 21.131 | 11.621 | 1.00 | 25.65 |
| ATOM | 4961 | CB | GLU | 728 | −9.052 | 21.331 | 12.623 | 1.00 | 28.30 |
| ATOM | 4962 | CG | GLU | 728 | −9.655 | 20.064 | 13.186 | 1.00 | 30.71 |
| ATOM | 4963 | CD | GLU | 728 | −10.802 | 20.348 | 14.159 | 1.00 | 33.29 |
| ATOM | 4964 | OE1 | GLU | 728 | −11.541 | 21.333 | 13.952 | 1.00 | 35.04 |
| ATOM | 4965 | OE2 | GLU | 728 | −10.979 | 19.579 | 15.124 | 1.00 | 32.42 |
| ATOM | 4966 | C | GLU | 728 | −6.570 | 21.013 | 12.353 | 1.00 | 24.86 |
| ATOM | 4967 | O | GLU | 728 | −6.069 | 21.993 | 12.898 | 1.00 | 25.47 |
| ATOM | 4968 | N | ARG | 729 | −5.986 | 19.816 | 12.353 | 1.00 | 24.34 |
| ATOM | 4969 | CA | ARG | 729 | −4.700 | 19.612 | 13.010 | 1.00 | 22.72 |
| ATOM | 4970 | CB | ARG | 729 | −4.757 | 18.372 | 13.907 | 1.00 | 20.17 |
| ATOM | 4971 | CG | ARG | 729 | −5.646 | 18.597 | 15.121 | 1.00 | 18.27 |
| ATOM | 4972 | CD | ARG | 729 | −5.986 | 17.326 | 15.876 | 1.00 | 16.32 |
| ATOM | 4973 | NE | ARG | 729 | −6.862 | 17.615 | 17.011 | 1.00 | 17.63 |
| ATOM | 4974 | CZ | ARG | 729 | −8.095 | 18.109 | 16.911 | 1.00 | 19.57 |
| ATOM | 4975 | NH1 | ARG | 729 | −8.621 | 18.374 | 15.725 | 1.00 | 20.07 |
| ATOM | 4976 | NH2 | ARG | 729 | −8.808 | 18.350 | 18.003 | 1.00 | 20.63 |
| ATOM | 4977 | C | ARG | 729 | −3.554 | 19.524 | 12.004 | 1.00 | 22.25 |
| ATOM | 4978 | O | ARG | 729 | −2.546 | 18.855 | 12.232 | 1.00 | 21.75 |
| ATOM | 4979 | N | ALA | 730 | −3.737 | 20.211 | 10.880 | 1.00 | 22.50 |
| ATOM | 4980 | CA | ALA | 730 | −2.732 | 20.296 | 9.825 | 1.00 | 21.57 |
| ATOM | 4981 | CB | ALA | 730 | −1.419 | 20.801 | 10.417 | 1.00 | 20.27 |
| ATOM | 4982 | C | ALA | 730 | −2.472 | 19.044 | 9.008 | 1.00 | 19.61 |
| ATOM | 4983 | O | ALA | 730 | −1.474 | 18.978 | 8.301 | 1.00 | 18.88 |
| ATOM | 4984 | N | VAL | 731 | −3.356 | 18.057 | 9.083 | 1.00 | 17.78 |
| ATOM | 4985 | CA | VAL | 731 | −3.136 | 16.846 | 8.308 | 1.00 | 17.66 |
| ATOM | 4986 | CB | VAL | 731 | −3.204 | 15.596 | 9.205 | 1.00 | 17.57 |
| ATOM | 4987 | CG1 | VAL | 731 | −2.985 | 14.339 | 8.363 | 1.00 | 16.76 |
| ATOM | 4988 | CG2 | VAL | 731 | −2.138 | 15.688 | 10.296 | 1.00 | 18.90 |
| ATOM | 4989 | C | VAL | 731 | −4.114 | 16.673 | 7.153 | 1.00 | 17.88 |
| ATOM | 4990 | O | VAL | 731 | −5.283 | 16.373 | 7.365 | 1.00 | 17.43 |
| ATOM | 4991 | N | PRO | 732 | −3.652 | 16.892 | 5.912 | 1.00 | 17.50 |
| ATOM | 4992 | CD | PRO | 732 | −2.351 | 17.419 | 5.475 | 1.00 | 19.31 |
| ATOM | 4993 | CA | PRO | 732 | −4.554 | 16.727 | 4.773 | 1.00 | 18.43 |
| ATOM | 4994 | CB | PRO | 732 | −3.771 | 17.325 | 3.607 | 1.00 | 19.15 |
| ATOM | 4995 | CG | PRO | 732 | −2.359 | 17.103 | 4.002 | 1.00 | 23.17 |
| ATOM | 4996 | C | PRO | 732 | −4.820 | 15.248 | 4.606 | 1.00 | 16.61 |
| ATOM | 4997 | O | PRO | 732 | −3.927 | 14.415 | 4.797 | 1.00 | 15.91 |
| ATOM | 4998 | N | VAL | 733 | −6.056 | 14.925 | 4.257 | 1.00 | 15.66 |
| ATOM | 4999 | CA | VAL | 733 | −6.466 | 13.538 | 4.101 | 1.00 | 16.50 |
| ATOM | 5000 | CB | VAL | 733 | −7.543 | 13.184 | 5.144 | 1.00 | 16.63 |
| ATOM | 5001 | CG1 | VAL | 733 | −7.868 | 11.690 | 5.069 | 1.00 | 21.58 |
| ATOM | 5002 | CG2 | VAL | 733 | −7.070 | 13.588 | 6.541 | 1.00 | 17.55 |
| ATOM | 5003 | C | VAL | 733 | −7.038 | 13.185 | 2.737 | 1.00 | 16.46 |
| ATOM | 5004 | O | VAL | 733 | −7.803 | 13.951 | 2.160 | 1.00 | 16.24 |
| ATOM | 5005 | N | CYS | 734 | −6.668 | 12.010 | 2.242 | 1.00 | 13.15 |
| ATOM | 5006 | CA | CYS | 734 | −7.184 | 11.490 | 0.983 | 1.00 | 13.80 |
| ATOM | 5007 | CB | CYS | 734 | −6.051 | 10.957 | 0.120 | 1.00 | 11.02 |
| ATOM | 5008 | SG | CYS | 734 | −6.600 | 10.069 | −1.355 | 1.00 | 15.01 |
| ATOM | 5009 | C | CYS | 734 | −8.075 | 10.324 | 1.397 | 1.00 | 14.24 |
| ATOM | 5010 | O | CYS | 734 | −7.641 | 9.453 | 2.150 | 1.00 | 16.01 |
| ATOM | 5011 | N | GLY | 735 | −9.314 | 10.323 | 0.921 | 1.00 | 12.03 |
| ATOM | 5012 | CA | GLY | 735 | −10.229 | 9.250 | 1.246 | 1.00 | 13.47 |
| ATOM | 5013 | C | GLY | 735 | −9.985 | 8.069 | 0.322 | 1.00 | 14.63 |
| ATOM | 5014 | O | GLY | 735 | −9.214 | 8.174 | −0.630 | 1.00 | 15.25 |
| ATOM | 5015 | N | HIS | 736 | −10.647 | 6.946 | 0.593 | 1.00 | 14.52 |
| ATOM | 5016 | CA | HIS | 736 | −10.484 | 5.736 | −0.216 | 1.00 | 14.30 |
| ATOM | 5017 | CB | HIS | 736 | −9.269 | 4.956 | 0.311 | 1.00 | 15.08 |
| ATOM | 5018 | CG | HIS | 736 | −8.839 | 3.797 | −0.542 | 1.00 | 16.75 |
| ATOM | 5019 | CD2 | HIS | 736 | −9.519 | 3.010 | −1.411 | 1.00 | 17.45 |
| ATOM | 5020 | ND1 | HIS | 736 | −7.559 | 3.287 | −0.488 | 1.00 | 16.99 |
| ATOM | 5021 | CE1 | HIS | 736 | −7.469 | 2.236 | −1.282 | 1.00 | 18.58 |
| ATOM | 5022 | NE2 | HIS | 736 | −8.645 | 2.045 | −1.854 | 1.00 | 16.34 |
| ATOM | 5023 | C | HIS | 736 | −11.768 | 4.919 | −0.089 | 1.00 | 14.46 |
| ATOM | 5024 | O | HIS | 736 | −12.096 | 4.441 | 1.000 | 1.00 | 14.94 |
| ATOM | 5025 | N | LEU | 737 | −12.497 | 4.777 | −1.198 | 1.00 | 16.18 |
| ATOM | 5026 | CA | LEU | 737 | −13.763 | 4.033 | −1.215 | 1.00 | 17.93 |
| ATOM | 5027 | CB | LEU | 737 | −14.946 | 4.992 | −1.415 | 1.00 | 16.80 |
| ATOM | 5028 | CG | LEU | 737 | −15.210 | 6.030 | −0.320 | 1.00 | 13.20 |
| ATOM | 5029 | CD1 | LEU | 737 | −16.249 | 7.044 | −0.807 | 1.00 | 13.79 |
| ATOM | 5030 | CD2 | LEU | 737 | −15.695 | 5.326 | 0.939 | 1.00 | 15.41 |
| ATOM | 5031 | C | LEU | 737 | −13.776 | 2.993 | −2.323 | 1.00 | 18.17 |
| ATOM | 5032 | O | LEU | 737 | −12.963 | 3.040 | −3.245 | 1.00 | 16.22 |
| ATOM | 5033 | N | GLY | 738 | −14.715 | 2.060 | −2.223 | 1.00 | 19.98 |
| ATOM | 5034 | CA | GLY | 738 | −14.822 | 1.006 | −3.209 | 1.00 | 22.29 |
| ATOM | 5035 | C | GLY | 738 | −14.154 | −0.215 | −2.623 | 1.00 | 23.99 |
| ATOM | 5036 | O | GLY | 738 | −14.432 | −0.588 | −1.487 | 1.00 | 23.25 |
| ATOM | 5037 | N | LEU | 739 | −13.257 | −0.822 | −3.390 | 1.00 | 25.38 |
| ATOM | 5038 | CA | LEU | 739 | −12.537 | −1.999 | −2.941 | 1.00 | 25.53 |
| ATOM | 5039 | CB | LEU | 739 | −11.955 | −2.731 | −4.152 | 1.00 | 26.77 |
| ATOM | 5040 | CG | LEU | 739 | −11.686 | −4.230 | −4.032 | 1.00 | 28.57 |
| ATOM | 5041 | CD1 | LEU | 739 | −10.971 | −4.722 | −5.291 | 1.00 | 29.59 |
| ATOM | 5042 | CD2 | LEU | 739 | −10.849 | −4.508 | −2.804 | 1.00 | 27.92 |
| ATOM | 5043 | C | LEU | 739 | −11.413 | −1.560 | −1.998 | 1.00 | 26.90 |
| ATOM | 5044 | O | LEU | 739 | −10.331 | −1.173 | −2.443 | 1.00 | 27.05 |
| ATOM | 5045 | N | THR | 740 | −11.686 | −1.605 | −0.697 | 1.00 | 25.51 |
| ATOM | 5046 | CA | THR | 740 | −10.708 | −1.226 | 0.319 | 1.00 | 26.30 |
| ATOM | 5047 | CB | THR | 740 | −11.399 | −0.547 | 1.514 | 1.00 | 25.07 |
| ATOM | 5048 | OG1 | THR | 740 | −12.585 | −1.272 | 1.852 | 1.00 | 26.66 |
| ATOM | 5049 | CG2 | THR | 740 | −11.783 | 0.886 | 1.170 | 1.00 | 28.04 |
| ATOM | 5050 | C | THR | 740 | −9.986 | −2.487 | 0.794 | 1.00 | 25.33 |
| ATOM | 5051 | O | THR | 740 | −10.522 | −3.252 | 1.594 | 1.00 | 26.61 |
| ATOM | 5052 | N | PRO | 741 | −8.748 | −2.704 | 0.312 | 1.00 | 25.06 |
| ATOM | 5053 | CD | PRO | 741 | −8.001 | −1.737 | −0.510 | 1.00 | 26.23 |
| ATOM | 5054 | CA | PRO | 741 | −7.909 | −3.862 | 0.641 | 1.00 | 22.16 |
| ATOM | 5055 | CB | PRO | 741 | −6.584 | −3.542 | −0.060 | 1.00 | 25.15 |
| ATOM | 5056 | CG | PRO | 741 | −6.588 | −2.048 | −0.144 | 1.00 | 28.32 |
| ATOM | 5057 | C | PRO | 741 | −7.736 | −4.223 | 2.111 | 1.00 | 20.97 |
| ATOM | 5058 | O | PRO | 741 | −7.560 | −5.397 | 2.441 | 1.00 | 20.75 |
| ATOM | 5059 | N | GLN | 742 | −7.783 | −3.241 | 3.003 | 1.00 | 18.98 |
| ATOM | 5060 | CA | GLN | 742 | −7.637 | −3.556 | 4.425 | 1.00 | 18.22 |
| ATOM | 5061 | CB | GLN | 742 | −7.516 | −2.273 | 5.246 | 1.00 | 18.22 |
| ATOM | 5062 | CG | GLN | 742 | −6.108 | −1.698 | 5.237 | 1.00 | 19.20 |
| ATOM | 5063 | CD | GLN | 742 | −6.037 | −0.295 | 5.799 | 1.00 | 21.99 |
| ATOM | 5064 | OE1 | GLN | 742 | −6.773 | 0.056 | 6.722 | 1.00 | 23.77 |
| ATOM | 5065 | NE2 | GLN | 742 | −5.130 | 0.513 | 5.258 | 1.00 | 19.08 |
| ATOM | 5066 | C | GLN | 742 | −8.813 | −4.398 | 4.916 | 1.00 | 17.75 |
| ATOM | 5067 | O | GLN | 742 | −8.659 | −5.212 | 5.826 | 1.00 | 17.97 |
| ATOM | 5068 | N | SER | 743 | −9.976 | −4.209 | 4.296 | 1.00 | 18.05 |
| ATOM | 5069 | CA | SER | 743 | −11.171 | −4.950 | 4.673 | 1.00 | 17.66 |
| ATOM | 5070 | CB | SER | 743 | −12.412 | −4.063 | 4.535 | 1.00 | 19.22 |
| ATOM | 5071 | OG | SER | 743 | −12.368 | −2.992 | 5.473 | 1.00 | 19.51 |
| ATOM | 5072 | C | SER | 743 | −11.347 | −6.222 | 3.849 | 1.00 | 18.63 |
| ATOM | 5073 | O | SER | 743 | −12.456 | −6.742 | 3.725 | 1.00 | 19.29 |
| ATOM | 5074 | N | VAL | 744 | −10.252 | −6.733 | 3.291 | 1.00 | 17.52 |
| ATOM | 5075 | CA | VAL | 744 | −10.320 | −7.949 | 2.483 | 1.00 | 18.17 |
| ATOM | 5076 | CB | VAL | 744 | −8.900 | −8.422 | 2.066 | 1.00 | 20.91 |
| ATOM | 5077 | CG1 | VAL | 744 | −8.072 | −8.777 | 3.294 | 1.00 | 20.41 |
| ATOM | 5078 | CG2 | VAL | 744 | −9.004 | −9.617 | 1.126 | 1.00 | 21.66 |
| ATOM | 5079 | C | VAL | 744 | −11.061 | −9.087 | 3.206 | 1.00 | 18.57 |
| ATOM | 5080 | O | VAL | 744 | −11.873 | −9.786 | 2.594 | 1.00 | 17.85 |
| ATOM | 5081 | N | ASN | 745 | −10.801 | −9.267 | 4.501 | 1.00 | 18.34 |
| ATOM | 5082 | CA | ASN | 745 | −11.451 | −10.337 | 5.256 | 1.00 | 18.99 |
| ATOM | 5083 | CB | ASN | 745 | −10.783 | −10.501 | 6.624 | 1.00 | 18.10 |
| ATOM | 5084 | CG | ASN | 745 | −9.306 | −10.825 | 6.505 | 1.00 | 18.24 |
| ATOM | 5085 | OD1 | ASN | 745 | −8.931 | −11.911 | 6.055 | 1.00 | 16.42 |
| ATOM | 5086 | ND2 | ASN | 745 | −8.462 | −9.882 | 6.890 | 1.00 | 16.58 |
| ATOM | 5087 | C | ASN | 745 | −12.947 | −10.100 | 5.426 | 1.00 | 20.36 |
| ATOM | 5088 | O | ASN | 745 | −13.718 | −11.046 | 5.590 | 1.00 | 19.24 |
| ATOM | 5089 | N | ILE | 746 | −13.352 | −8.835 | 5.385 | 1.00 | 21.75 |
| ATOM | 5090 | CA | ILE | 746 | −14.758 | −8.474 | 5.512 | 1.00 | 24.03 |
| ATOM | 5091 | CB | ILE | 746 | −14.928 | −6.955 | 5.722 | 1.00 | 25.51 |
| ATOM | 5092 | CG2 | ILE | 746 | −16.393 | −6.570 | 5.577 | 1.00 | 27.38 |
| ATOM | 5093 | CG1 | ILE | 746 | −14.406 | −6.553 | 7.103 | 1.00 | 25.99 |
| ATOM | 5094 | CD1 | ILE | 746 | −15.274 | −7.038 | 8.241 | 1.00 | 23.57 |
| ATOM | 5095 | C | ILE | 746 | −15.481 | −8.866 | 4.233 | 1.00 | 25.01 |
| ATOM | 5096 | O | ILE | 746 | −16.510 | −9.537 | 4.274 | 1.00 | 24.42 |
| ATOM | 5097 | N | PHE | 747 | −14.927 | −8.450 | 3.098 | 1.00 | 26.24 |
| ATOM | 5098 | CA | PHE | 747 | −15.521 | −8.743 | 1.796 | 1.00 | 27.97 |
| ATOM | 5099 | CB | PHE | 747 | −14.900 | −7.859 | 0.709 | 1.00 | 30.22 |
| ATOM | 5100 | CG | PHE | 747 | −14.863 | −6.396 | 1.056 | 1.00 | 33.12 |
| ATOM | 5101 | CD1 | PHE | 747 | −16.006 | −5.739 | 1.504 | 1.00 | 34.45 |
| ATOM | 5102 | CD2 | PHE | 747 | −13.680 | −5.672 | 0.924 | 1.00 | 34.85 |
| ATOM | 5103 | CE1 | PHE | 747 | −15.970 | −4.380 | 1.819 | 1.00 | 35.58 |
| ATOM | 5104 | CE2 | PHE | 747 | −13.631 | −4.313 | 1.235 | 1.00 | 35.40 |
| ATOM | 5105 | CZ | PHE | 747 | −14.779 | −3.668 | 1.683 | 1.00 | 35.63 |
| ATOM | 5106 | C | PHE | 747 | −15.323 | −10.203 | 1.412 | 1.00 | 29.09 |
| ATOM | 5107 | O | PHE | 747 | −16.061 | −10.742 | 0.587 | 1.00 | 28.25 |
| ATOM | 5108 | N | GLY | 748 | −14.317 | −10.837 | 2.006 | 1.00 | 29.00 |
| ATOM | 5109 | CA | GLY | 748 | −14.042 | −12.229 | 1.698 | 1.00 | 29.92 |
| ATOM | 5110 | C | GLY | 748 | −13.185 | −12.347 | 0.453 | 1.00 | 30.99 |
| ATOM | 5111 | O | GLY | 748 | −13.228 | −13.354 | −0.253 | 1.00 | 33.17 |
| ATOM | 5112 | N | GLY | 749 | −12.400 | −11.309 | 0.184 | 1.00 | 30.50 |
| ATOM | 5113 | CA | GLY | 749 | −11.539 | −11.309 | −0.985 | 1.00 | 31.40 |
| ATOM | 5114 | C | GLY | 749 | −11.590 | −9.973 | −1.705 | 1.00 | 33.02 |
| ATOM | 5115 | O | GLY | 749 | −12.279 | −9.052 | −1.269 | 1.00 | 31.79 |
| ATOM | 5116 | N | TYR | 750 | −10.857 | −9.862 | −2.807 | 1.00 | 33.78 |
| ATOM | 5117 | CA | TYR | 750 | −10.839 | −8.626 | −3.574 | 1.00 | 35.38 |
| ATOM | 5118 | CB | TYR | 750 | −9.440 | −8.377 | −4.133 | 1.00 | 36.26 |
| ATOM | 5119 | CG | TYR | 750 | −8.361 | −8.445 | −3.076 | 1.00 | 38.04 |
| ATOM | 5120 | CD1 | TYR | 750 | −7.861 | −9.674 | −2.639 | 1.00 | 39.14 |
| ATOM | 5121 | CE1 | TYR | 750 | −6.886 | −9.742 | −1.644 | 1.00 | 39.39 |
| ATOM | 5122 | CD2 | TYR | 750 | −7.858 | −7.283 | −2.490 | 1.00 | 38.52 |
| ATOM | 5123 | CE2 | TYR | 750 | −6.886 | −7.339 | −1.493 | 1.00 | 39.18 |
| ATOM | 5124 | CZ | TYR | 750 | −6.404 | −8.569 | −1.077 | 1.00 | 39.42 |
| ATOM | 5125 | OH | TYR | 750 | −5.441 | −8.626 | −0.095 | 1.00 | 39.23 |
| ATOM | 5126 | C | TYR | 750 | −11.856 | −8.715 | −4.705 | 1.00 | 35.70 |
| ATOM | 5127 | O | TYR | 750 | −11.591 | −9.304 | −5.753 | 1.00 | 36.41 |
| ATOM | 5128 | N | LYS | 751 | −13.025 | −8.127 | −4.480 | 1.00 | 35.48 |
| ATOM | 5129 | CA | LYS | 751 | −14.098 | −8.156 | −5.463 | 1.00 | 36.67 |
| ATOM | 5130 | CB | LYS | 751 | −15.255 | −8.989 | −4.911 | 1.00 | 38.25 |
| ATOM | 5131 | CG | LYS | 751 | −14.811 | −10.343 | −4.361 | 1.00 | 40.62 |
| ATOM | 5132 | CD | LYS | 751 | −15.870 | −10.976 | −3.466 | 1.00 | 42.04 |
| ATOM | 5133 | CE | LYS | 751 | −15.344 | −12.243 | −2.802 | 1.00 | 42.02 |
| ATOM | 5134 | NZ | LYS | 751 | −16.334 | −12.839 | −1.854 | 1.00 | 42.38 |
| ATOM | 5135 | C | LYS | 751 | −14.569 | −6.741 | −5.783 | 1.00 | 36.24 |
| ATOM | 5136 | O | LYS | 751 | −14.371 | −5.819 | −4.993 | 1.00 | 36.25 |
| ATOM | 5137 | N | VAL | 752 | −15.194 | −6.573 | −6.943 | 1.00 | 36.26 |
| ATOM | 5138 | CA | VAL | 752 | −15.687 | −5.264 | −7.357 | 1.00 | 36.20 |
| ATOM | 5139 | CB | VAL | 752 | −16.196 | −5.296 | −8.816 | 1.00 | 36.16 |
| ATOM | 5140 | CG1 | VAL | 752 | −16.794 | −3.948 | −9.191 | 1.00 | 36.40 |
| ATOM | 5141 | CG2 | VAL | 752 | −15.057 | −5.641 | −9.753 | 1.00 | 35.98 |
| ATOM | 5142 | C | VAL | 752 | −16.826 | −4.784 | −6.461 | 1.00 | 36.45 |
| ATOM | 5143 | O | VAL | 752 | −17.758 | −5.533 | −6.177 | 1.00 | 36.56 |
| ATOM | 5144 | N | GLN | 753 | −16.740 | −3.533 | −6.018 | 1.00 | 36.02 |
| ATOM | 5145 | CA | GLN | 753 | −17.772 | −2.943 | −5.169 | 1.00 | 36.40 |
| ATOM | 5146 | CB | GLN | 753 | −17.156 | −2.319 | −3.915 | 1.00 | 37.30 |
| ATOM | 5147 | CG | GLN | 753 | −17.031 | −3.264 | −2.730 | 1.00 | 42.36 |
| ATOM | 5148 | CD | GLN | 753 | −15.996 | −4.345 | −2.946 | 1.00 | 44.63 |
| ATOM | 5149 | OE1 | GLN | 753 | −14.816 | −4.055 | −3.155 | 1.00 | 46.92 |
| ATOM | 5150 | NE2 | GLN | 753 | −16.428 | −5.600 | −2.889 | 1.00 | 44.11 |
| ATOM | 5151 | C | GLN | 753 | −18.540 | −1.875 | −5.937 | 1.00 | 35.82 |
| ATOM | 5152 | O | GLN | 753 | −18.138 | −1.474 | −7.028 | 1.00 | 34.76 |
| ATOM | 5153 | N | GLY | 754 | −19.645 | −1.417 | −5.361 | 1.00 | 36.54 |
| ATOM | 5154 | CA | GLY | 754 | −20.446 | −0.397 | −6.015 | 1.00 | 37.86 |
| ATOM | 5155 | C | GLY | 754 | −21.571 | −0.979 | −6.848 | 1.00 | 38.80 |
| ATOM | 5156 | O | GLY | 754 | −22.558 | −0.301 | −7.136 | 1.00 | 38.37 |
| ATOM | 5157 | N | ARG | 755 | −21.413 | −2.240 | −7.237 | 1.00 | 40.53 |
| ATOM | 5158 | CA | ARG | 755 | −22.405 | −2.948 | −8.037 | 1.00 | 42.45 |
| ATOM | 5159 | CB | ARG | 755 | −22.018 | −4.428 | −8.145 | 1.00 | 43.43 |
| ATOM | 5160 | CG | ARG | 755 | −20.750 | −4.711 | −8.951 | 1.00 | 44.60 |
| ATOM | 5161 | CD | ARG | 755 | −21.069 | −4.927 | −10.423 | 1.00 | 44.87 |
| ATOM | 5162 | NE | ARG | 755 | −19.883 | −5.181 | −11.242 | 1.00 | 44.38 |
| ATOM | 5163 | CZ | ARG | 755 | −19.041 | −6.196 | −11.066 | 1.00 | 45.22 |
| ATOM | 5164 | NH1 | ARG | 755 | −19.240 | −7.072 | −10.090 | 1.00 | 45.18 |
| ATOM | 5165 | NH2 | ARG | 755 | −18.001 | −6.343 | −11.876 | 1.00 | 44.29 |
| ATOM | 5166 | C | ARG | 755 | −23.788 | −2.826 | −7.399 | 1.00 | 42.83 |
| ATOM | 5167 | O | ARG | 755 | −23.990 | −3.237 | −6.257 | 1.00 | 43.19 |
| ATOM | 5168 | N | GLY | 756 | −24.734 | −2.257 | −8.139 | 1.00 | 43.66 |
| ATOM | 5169 | CA | GLY | 756 | −26.079 | −2.103 | −7.616 | 1.00 | 44.45 |
| ATOM | 5170 | C | GLY | 756 | −26.404 | −0.696 | −7.154 | 1.00 | 45.42 |
| ATOM | 5171 | O | GLY | 756 | −25.510 | 0.087 | −6.827 | 1.00 | 45.05 |
| ATOM | 5172 | N | ASP | 757 | −27.693 | −0.375 | −7.123 | 1.00 | 45.36 |
| ATOM | 5173 | CA | ASP | 757 | −28.151 | 0.944 | −6.704 | 1.00 | 45.65 |
| ATOM | 5174 | CB | ASP | 757 | −29.642 | 1.095 | −7.011 | 1.00 | 48.39 |
| ATOM | 5175 | CG | ASP | 757 | −29.954 | 0.882 | −8.477 | 1.00 | 50.42 |
| ATOM | 5176 | OD1 | ASP | 757 | −29.426 | 1.648 | −9.312 | 1.00 | 51.37 |
| ATOM | 5177 | OD2 | ASP | 757 | −30.725 | −0.051 | −8.795 | 1.00 | 51.94 |
| ATOM | 5178 | C | ASP | 757 | −27.898 | 1.188 | −5.219 | 1.00 | 43.87 |
| ATOM | 5179 | O | ASP | 757 | −27.445 | 2.264 | −4.827 | 1.00 | 43.00 |
| ATOM | 5180 | N | GLU | 758 | −28.193 | 0.187 | −4.396 | 1.00 | 42.25 |
| ATOM | 5181 | CA | GLU | 758 | −27.988 | 0.303 | −2.956 | 1.00 | 41.44 |
| ATOM | 5182 | CB | GLU | 758 | −28.338 | −1.013 | −2.260 | 1.00 | 43.32 |
| ATOM | 5183 | CG | GLU | 758 | −28.241 | −0.945 | −0.744 | 1.00 | 47.39 |
| ATOM | 5184 | CD | GLU | 758 | −27.797 | −2.259 | −0.123 | 1.00 | 49.85 |
| ATOM | 5185 | OE1 | GLU | 758 | −28.446 | −3.298 | −0.389 | 1.00 | 50.43 |
| ATOM | 5186 | OE2 | GLU | 758 | −26.796 | −2.248 | 0.632 | 1.00 | 50.64 |
| ATOM | 5187 | C | GLU | 758 | −26.532 | 0.653 | −2.653 | 1.00 | 39.44 |
| ATOM | 5188 | O | GLU | 758 | −26.242 | 1.628 | −1.956 | 1.00 | 37.55 |
| ATOM | 5189 | N | ALA | 759 | −25.622 | −0.160 | −3.180 | 1.00 | 37.40 |
| ATOM | 5190 | CA | ALA | 759 | −24.191 | 0.037 | −2.981 | 1.00 | 34.93 |
| ATOM | 5191 | CB | ALA | 759 | −23.420 | −1.141 | −3.568 | 1.00 | 35.45 |
| ATOM | 5192 | C | ALA | 759 | −23.735 | 1.332 | −3.639 | 1.00 | 33.03 |
| ATOM | 5193 | O | ALA | 759 | −22.993 | 2.114 | −3.045 | 1.00 | 31.60 |
| ATOM | 5194 | N | GLY | 760 | −24.182 | 1.541 | −4.873 | 1.00 | 31.34 |
| ATOM | 5195 | CA | GLY | 760 | −23.819 | 2.735 | −5.610 | 1.00 | 29.95 |
| ATOM | 5196 | C | GLY | 760 | −24.143 | 4.016 | −4.868 | 1.00 | 29.45 |
| ATOM | 5197 | O | GLY | 760 | −23.272 | 4.866 | −4.684 | 1.00 | 28.24 |
| ATOM | 5198 | N | ASP | 761 | −25.396 | 4.152 | −4.440 | 1.00 | 28.80 |
| ATOM | 5199 | CA | ASP | 761 | −25.840 | 5.335 | −3.714 | 1.00 | 28.80 |
| ATOM | 5200 | CB | ASP | 761 | −27.343 | 5.248 | −3.415 | 1.00 | 29.11 |
| ATOM | 5201 | CG | ASP | 761 | −28.193 | 5.146 | −4.676 | 1.00 | 29.37 |
| ATOM | 5202 | OD1 | ASP | 761 | −27.664 | 5.376 | −5.782 | 1.00 | 29.25 |
| ATOM | 5203 | OD2 | ASP | 761 | −29.400 | 4.845 | −4.556 | 1.00 | 31.42 |
| ATOM | 5204 | C | ASP | 761 | −25.070 | 5.494 | −2.410 | 1.00 | 28.23 |
| ATOM | 5205 | O | ASP | 761 | −24.802 | 6.612 | −1.960 | 1.00 | 26.71 |
| ATOM | 5206 | N | GLN | 762 | −24.717 | 4.369 | −1.798 | 1.00 | 28.59 |
| ATOM | 5207 | CA | GLN | 762 | −23.970 | 4.396 | −0.546 | 1.00 | 28.32 |
| ATOM | 5208 | CB | GLN | 762 | −23.858 | 2.988 | 0.040 | 1.00 | 30.23 |
| ATOM | 5209 | CG | GLN | 762 | −23.020 | 2.931 | 1.305 | 1.00 | 32.27 |
| ATOM | 5210 | CD | GLN | 762 | −23.535 | 3.860 | 2.378 | 1.00 | 33.99 |
| ATOM | 5211 | OE1 | GLN | 762 | −24.663 | 3.718 | 2.852 | 1.00 | 36.79 |
| ATOM | 5212 | NE2 | GLN | 762 | −22.712 | 4.825 | 2.767 | 1.00 | 35.03 |
| ATOM | 5213 | C | GLN | 762 | −22.571 | 4.973 | −0.754 | 1.00 | 26.19 |
| ATOM | 5214 | O | GLN | 762 | −22.091 | 5.757 | 0.065 | 1.00 | 24.24 |
| ATOM | 5215 | N | LEU | 763 | −21.917 | 4.578 | −1.842 | 1.00 | 25.52 |
| ATOM | 5216 | CA | LEU | 763 | −20.575 | 5.078 | −2.135 | 1.00 | 25.74 |
| ATOM | 5217 | CB | LEU | 763 | −19.971 | 4.340 | −3.337 | 1.00 | 28.01 |
| ATOM | 5218 | CG | LEU | 763 | −19.364 | 2.960 | −3.070 | 1.00 | 30.99 |
| ATOM | 5219 | CD1 | LEU | 763 | −18.850 | 2.357 | −4.373 | 1.00 | 31.68 |
| ATOM | 5220 | CD2 | LEU | 763 | −18.224 | 3.087 | −2.071 | 1.00 | 30.71 |
| ATOM | 5221 | C | LEU | 763 | −20.602 | 6.576 | −2.416 | 1.00 | 24.34 |
| ATOM | 5222 | O | LEU | 763 | −19.725 | 7.319 | −1.974 | 1.00 | 20.25 |
| ATOM | 5223 | N | LEU | 764 | −21.616 | 7.013 | −3.154 | 1.00 | 24.14 |
| ATOM | 5224 | CA | LEU | 764 | −21.761 | 8.424 | −3.488 | 1.00 | 24.45 |
| ATOM | 5225 | CB | LEU | 764 | −22.988 | 8.633 | −4.380 | 1.00 | 24.40 |
| ATOM | 5226 | CG | LEU | 764 | −22.910 | 9.711 | −5.461 | 1.00 | 26.78 |
| ATOM | 5227 | CD1 | LEU | 764 | −24.307 | 9.955 | −6.006 | 1.00 | 27.16 |
| ATOM | 5228 | CD2 | LEU | 764 | −22.323 | 10.988 | −4.906 | 1.00 | 26.33 |
| ATOM | 5229 | C | LEU | 764 | −21.926 | 9.216 | −2.192 | 1.00 | 22.36 |
| ATOM | 5230 | O | LEU | 764 | −21.257 | 10.225 | −1.978 | 1.00 | 21.39 |
| ATOM | 5231 | N | SER | 765 | −22.823 | 8.748 | −1.330 | 1.00 | 22.67 |
| ATOM | 5232 | CA | SER | 765 | −23.064 | 9.415 | −0.055 | 1.00 | 21.77 |
| ATOM | 5233 | CB | SER | 765 | −24.134 | 8.676 | 0.745 | 1.00 | 21.69 |
| ATOM | 5234 | OG | SER | 765 | −24.403 | 9.355 | 1.955 | 1.00 | 24.43 |
| ATOM | 5235 | C | SER | 765 | −21.779 | 9.486 | 0.766 | 1.00 | 20.68 |
| ATOM | 5236 | O | SER | 765 | −21.459 | 10.527 | 1.337 | 1.00 | 18.30 |
| ATOM | 5237 | N | ASP | 766 | −21.050 | 8.373 | 0.832 | 1.00 | 19.16 |
| ATOM | 5238 | CA | ASP | 766 | −19.801 | 8.338 | 1.585 | 1.00 | 19.55 |
| ATOM | 5239 | CB | ASP | 766 | −19.213 | 6.917 | 1.623 | 1.00 | 19.61 |
| ATOM | 5240 | CG | ASP | 766 | −19.907 | 6.011 | 2.643 | 1.00 | 23.80 |
| ATOM | 5241 | OD1 | ASP | 766 | −20.473 | 6.524 | 3.632 | 1.00 | 20.16 |
| ATOM | 5242 | OD2 | ASP | 766 | −19.863 | 4.774 | 2.458 | 1.00 | 24.88 |
| ATOM | 5243 | C | ASP | 766 | −18.768 | 9.295 | 0.993 | 1.00 | 17.74 |
| ATOM | 5244 | O | ASP | 766 | −18.074 | 9.998 | 1.727 | 1.00 | 17.68 |
| ATOM | 5245 | N | ALA | 767 | −18.666 | 9.321 | −0.332 | 1.00 | 18.82 |
| ATOM | 5246 | CA | ALA | 767 | −17.708 | 10.200 | −0.993 | 1.00 | 18.71 |
| ATOM | 5247 | CB | ALA | 767 | −17.759 | 9.992 | −2.501 | 1.00 | 18.07 |
| ATOM | 5248 | C | ALA | 767 | −17.992 | 11.658 | −0.647 | 1.00 | 18.35 |
| ATOM | 5249 | O | ALA | 767 | −17.081 | 12.408 | −0.289 | 1.00 | 17.25 |
| ATOM | 5250 | N | LEU | 768 | −19.260 | 12.052 | −0.745 | 1.00 | 18.76 |
| ATOM | 5251 | CA | LEU | 768 | −19.674 | 13.421 | −0.437 | 1.00 | 18.68 |
| ATOM | 5252 | CB | LEU | 768 | −21.150 | 13.617 | −0.796 | 1.00 | 19.27 |
| ATOM | 5253 | CG | LEU | 768 | −21.490 | 13.710 | −2.291 | 1.00 | 19.38 |
| ATOM | 5254 | CD1 | LEU | 768 | −22.991 | 13.530 | −2.498 | 1.00 | 18.38 |
| ATOM | 5255 | CD2 | LEU | 768 | −21.036 | 15.068 | −2.823 | 1.00 | 21.35 |
| ATOM | 5256 | C | LEU | 768 | −19.465 | 13.744 | 1.033 | 1.00 | 18.16 |
| ATOM | 5257 | O | LEU | 768 | −19.127 | 14.878 | 1.392 | 1.00 | 18.53 |
| ATOM | 5258 | N | ALA | 769 | −19.669 | 12.746 | 1.886 | 1.00 | 15.98 |
| ATOM | 5259 | CA | ALA | 769 | −19.502 | 12.936 | 3.319 | 1.00 | 15.17 |
| ATOM | 5260 | CB | ALA | 769 | −20.038 | 11.726 | 4.069 | 1.00 | 17.07 |
| ATOM | 5261 | C | ALA | 769 | −18.033 | 13.166 | 3.661 | 1.00 | 14.89 |
| ATOM | 5262 | O | ALA | 769 | −17.714 | 13.986 | 4.519 | 1.00 | 16.01 |
| ATOM | 5263 | N | LEU | 770 | −17.146 | 12.445 | 2.979 | 1.00 | 13.79 |
| ATOM | 5264 | CA | LEU | 770 | −15.710 | 12.577 | 3.211 | 1.00 | 14.28 |
| ATOM | 5265 | CB | LEU | 770 | −14.947 | 11.464 | 2.482 | 1.00 | 13.69 |
| ATOM | 5266 | CG | LEU | 770 | −15.159 | 10.031 | 3.022 | 1.00 | 14.90 |
| ATOM | 5267 | CD1 | LEU | 770 | −14.652 | 8.989 | 2.028 | 1.00 | 14.19 |
| ATOM | 5268 | CD2 | LEU | 770 | −14.439 | 9.898 | 4.346 | 1.00 | 14.20 |
| ATOM | 5269 | C | LEU | 770 | −15.250 | 13.940 | 2.713 | 1.00 | 14.23 |
| ATOM | 5270 | O | LEU | 770 | −14.387 | 14.585 | 3.319 | 1.00 | 15.29 |
| ATOM | 5271 | N | GLU | 771 | −15.826 | 14.385 | 1.601 | 1.00 | 15.62 |
| ATOM | 5272 | CA | GLU | 771 | −15.457 | 15.685 | 1.049 | 1.00 | 14.95 |
| ATOM | 5273 | CB | GLU | 771 | −16.126 | 15.892 | −0.311 | 1.00 | 13.82 |
| ATOM | 5274 | CG | GLU | 771 | −15.885 | 17.276 | −0.887 | 1.00 | 14.20 |
| ATOM | 5275 | CD | GLU | 771 | −16.600 | 17.471 | −2.201 | 1.00 | 16.87 |
| ATOM | 5276 | OE1 | GLU | 771 | −17.837 | 17.270 | −2.243 | 1.00 | 16.35 |
| ATOM | 5277 | OE2 | GLU | 771 | −15.931 | 17.824 | −3.184 | 1.00 | 16.38 |
| ATOM | 5278 | C | GLU | 771 | −15.874 | 16.793 | 2.015 | 1.00 | 15.89 |
| ATOM | 5279 | O | GLU | 771 | −15.097 | 17.714 | 2.301 | 1.00 | 15.83 |
| ATOM | 5280 | N | ALA | 772 | −17.104 | 16.696 | 2.512 | 1.00 | 15.87 |
| ATOM | 5281 | CA | ALA | 772 | −17.653 | 17.677 | 3.449 | 1.00 | 17.16 |
| ATOM | 5282 | CB | ALA | 772 | −19.133 | 17.384 | 3.695 | 1.00 | 16.98 |
| ATOM | 5283 | C | ALA | 772 | −16.896 | 17.678 | 4.776 | 1.00 | 18.66 |
| ATOM | 5284 | O | ALA | 772 | −16.855 | 18.695 | 5.472 | 1.00 | 17.21 |
| ATOM | 5285 | N | ALA | 773 | −16.294 | 16.537 | 5.115 | 1.00 | 18.43 |
| ATOM | 5286 | CA | ALA | 773 | −15.536 | 16.392 | 6.358 | 1.00 | 18.51 |
| ATOM | 5287 | CB | ALA | 773 | −15.359 | 14.909 | 6.690 | 1.00 | 17.94 |
| ATOM | 5288 | C | ALA | 773 | −14.170 | 17.067 | 6.265 | 1.00 | 17.57 |
| ATOM | 5289 | O | ALA | 773 | −13.539 | 17.358 | 7.284 | 1.00 | 19.73 |
| ATOM | 5290 | N | GLY | 774 | −13.714 | 17.309 | 5.040 | 1.00 | 18.75 |
| ATOM | 5291 | CA | GLY | 774 | −12.424 | 17.954 | 4.853 | 1.00 | 16.12 |
| ATOM | 5292 | C | GLY | 774 | −11.451 | 17.251 | 3.913 | 1.00 | 15.01 |
| ATOM | 5293 | O | GLY | 774 | −10.395 | 17.797 | 3.600 | 1.00 | 13.07 |
| ATOM | 5294 | N | ALA | 775 | −11.786 | 16.048 | 3.451 | 1.00 | 15.01 |
| ATOM | 5295 | CA | ALA | 775 | −10.893 | 15.326 | 2.546 | 1.00 | 15.38 |
| ATOM | 5296 | CB | ALA | 775 | −11.513 | 13.990 | 2.134 | 1.00 | 15.63 |
| ATOM | 5297 | C | ALA | 775 | −10.610 | 16.171 | 1.308 | 1.00 | 15.18 |
| ATOM | 5298 | O | ALA | 775 | −11.534 | 16.705 | 0.708 | 1.00 | 14.76 |
| ATOM | 5299 | N | GLN | 776 | −9.337 | 16.270 | 0.924 | 1.00 | 14.50 |
| ATOM | 5300 | CA | GLN | 776 | −8.934 | 17.071 | −0.230 | 1.00 | 15.43 |
| ATOM | 5301 | CB | GLN | 776 | −7.695 | 17.893 | 0.129 | 1.00 | 15.10 |
| ATOM | 5302 | CG | GLN | 776 | −7.928 | 18.846 | 1.283 | 1.00 | 18.62 |
| ATOM | 5303 | CD | GLN | 776 | −6.658 | 19.513 | 1.747 | 1.00 | 18.59 |
| ATOM | 5304 | OE1 | GLN | 776 | −6.002 | 20.221 | 0.984 | 1.00 | 24.99 |
| ATOM | 5305 | NE2 | GLN | 776 | −6.305 | 19.298 | 3.007 | 1.00 | 21.94 |
| ATOM | 5306 | C | GLN | 776 | −8.657 | 16.233 | −1.474 | 1.00 | 15.17 |
| ATOM | 5307 | O | GLN | 776 | −8.344 | 16.765 | −2.537 | 1.00 | 14.62 |
| ATOM | 5308 | N | LEU | 777 | −8.767 | 14.918 | −1.330 | 1.00 | 15.85 |
| ATOM | 5309 | CA | LEU | 777 | −8.547 | 13.990 | −2.429 | 1.00 | 15.20 |
| ATOM | 5310 | CB | LEU | 777 | −7.057 | 13.673 | −2.562 | 1.00 | 16.16 |
| ATOM | 5311 | CG | LEU | 777 | −6.264 | 14.319 | −3.689 | 1.00 | 22.07 |
| ATOM | 5312 | CD1 | LEU | 777 | −4.776 | 13.947 | −3.529 | 1.00 | 20.46 |
| ATOM | 5313 | CD2 | LEU | 777 | −6.792 | 13.831 | −5.042 | 1.00 | 23.78 |
| ATOM | 5314 | C | LEU | 777 | −9.316 | 12.710 | −2.125 | 1.00 | 15.07 |
| ATOM | 5315 | O | LEU | 777 | −9.600 | 12.419 | −0.966 | 1.00 | 12.13 |
| ATOM | 5316 | N | LEU | 778 | −9.654 | 11.954 | −3.163 | 1.00 | 13.83 |
| ATOM | 5317 | CA | LEU | 778 | −10.375 | 10.701 | −2.979 | 1.00 | 14.56 |
| ATOM | 5318 | CB | LEU | 778 | −11.891 | 10.887 | −3.176 | 1.00 | 14.01 |
| ATOM | 5319 | CG | LEU | 778 | −12.722 | 9.593 | −3.192 | 1.00 | 14.97 |
| ATOM | 5320 | CD1 | LEU | 778 | −12.716 | 8.948 | −1.806 | 1.00 | 15.87 |
| ATOM | 5321 | CD2 | LEU | 778 | −14.150 | 9.893 | −3.609 | 1.00 | 13.98 |
| ATOM | 5322 | C | LEU | 778 | −9.892 | 9.647 | −3.955 | 1.00 | 15.86 |
| ATOM | 5323 | O | LEU | 778 | −9.731 | 9.913 | −5.150 | 1.00 | 16.74 |
| ATOM | 5324 | N | VAL | 779 | −9.639 | 8.450 | −3.440 | 1.00 | 14.91 |
| ATOM | 5325 | CA | VAL | 779 | −9.235 | 7.348 | −4.295 | 1.00 | 15.20 |
| ATOM | 5326 | CB | VAL | 779 | −8.057 | 6.528 | −3.694 | 1.00 | 15.02 |
| ATOM | 5327 | CG1 | VAL | 779 | −7.850 | 5.242 | −4.509 | 1.00 | 14.33 |
| ATOM | 5328 | CG2 | VAL | 779 | −6.774 | 7.354 | −3.730 | 1.00 | 15.87 |
| ATOM | 5329 | C | VAL | 779 | −10.465 | 6.453 | −4.416 | 1.00 | 15.26 |
| ATOM | 5330 | O | VAL | 779 | −11.171 | 6.222 | −3.430 | 1.00 | 14.26 |
| ATOM | 5331 | N | LEU | 780 | −10.725 | 5.982 | −5.632 | 1.00 | 16.87 |
| ATOM | 5332 | CA | LEU | 780 | −11.854 | 5.094 | −5.928 | 1.00 | 18.23 |
| ATOM | 5333 | CB | LEU | 780 | −12.807 | 5.737 | −6.937 | 1.00 | 20.90 |
| ATOM | 5334 | CG | LEU | 780 | −14.160 | 6.196 | −6.413 | 1.00 | 24.41 |
| ATOM | 5335 | CD1 | LEU | 780 | −15.027 | 6.667 | −7.586 | 1.00 | 22.61 |
| ATOM | 5336 | CD2 | LEU | 780 | −14.841 | 5.046 | −5.677 | 1.00 | 24.16 |
| ATOM | 5337 | C | LEU | 780 | −11.263 | 3.841 | −6.535 | 1.00 | 17.70 |
| ATOM | 5338 | O | LEU | 780 | −10.617 | 3.912 | −7.580 | 1.00 | 18.28 |
| ATOM | 5339 | N | GLU | 781 | −11.485 | 2.693 | −5.895 | 1.00 | 17.11 |
| ATOM | 5340 | CA | GLU | 781 | −10.913 | 1.434 | −6.386 | 1.00 | 18.49 |
| ATOM | 5341 | CB | GLU | 781 | −9.972 | 0.857 | −5.314 | 1.00 | 19.29 |
| ATOM | 5342 | CG | GLU | 781 | −9.242 | −0.416 | −5.728 | 1.00 | 19.49 |
| ATOM | 5343 | CD | GLU | 781 | −8.175 | −0.831 | −4.738 | 1.00 | 23.53 |
| ATOM | 5344 | OE1 | GLU | 781 | −7.901 | −0.070 | −3.781 | 1.00 | 21.13 |
| ATOM | 5345 | OE2 | GLU | 781 | −7.597 | −1.925 | −4.921 | 1.00 | 25.81 |
| ATOM | 5346 | C | GLU | 781 | −11.930 | 0.370 | −6.808 | 1.00 | 20.03 |
| ATOM | 5347 | O | GLU | 781 | −12.883 | 0.091 | −6.088 | 1.00 | 21.13 |
| ATOM | 5348 | N | CYS | 782 | −11.701 | −0.207 | −7.986 | 1.00 | 21.54 |
| ATOM | 5349 | CA | CYS | 782 | −12.535 | −1.260 | −8.555 | 1.00 | 22.35 |
| ATOM | 5350 | CB | CYS | 782 | −12.071 | −2.623 | −8.037 | 1.00 | 22.48 |
| ATOM | 5351 | SG | CYS | 782 | −10.365 | −2.991 | −8.514 | 1.00 | 23.47 |
| ATOM | 5352 | C | CYS | 782 | −14.024 | −1.076 | −8.314 | 1.00 | 23.36 |
| ATOM | 5353 | O | CYS | 782 | −14.639 | −1.733 | −7.469 | 1.00 | 22.95 |
| ATOM | 5354 | N | VAL | 783 | −14.586 | −0.163 | −9.088 | 1.00 | 23.52 |
| ATOM | 5355 | CA | VAL | 783 | −15.988 | 0.173 | −9.016 | 1.00 | 26.91 |
| ATOM | 5356 | CB | VAL | 783 | −16.170 | 1.481 | −8.199 | 1.00 | 28.96 |
| ATOM | 5357 | CG1 | VAL | 783 | −15.616 | 2.665 | −8.975 | 1.00 | 29.79 |
| ATOM | 5358 | CG2 | VAL | 783 | −17.622 | 1.687 | −7.843 | 1.00 | 32.62 |
| ATOM | 5359 | C | VAL | 783 | −16.434 | 0.360 | −10.467 | 1.00 | 26.23 |
| ATOM | 5360 | O | VAL | 783 | −15.628 | 0.711 | −11.326 | 1.00 | 26.43 |
| ATOM | 5361 | N | PRO | 784 | −17.711 | 0.083 | −10.770 | 1.00 | 26.23 |
| ATOM | 5362 | CD | PRO | 784 | −18.792 | −0.423 | −9.913 | 1.00 | 27.16 |
| ATOM | 5363 | CA | PRO | 784 | −18.177 | 0.256 | −12.149 | 1.00 | 26.47 |
| ATOM | 5364 | CB | PRO | 784 | −19.671 | −0.087 | −12.060 | 1.00 | 25.76 |
| ATOM | 5365 | CG | PRO | 784 | −20.008 | 0.120 | −10.612 | 1.00 | 27.94 |
| ATOM | 5366 | C | PRO | 784 | −17.908 | 1.675 | −12.644 | 1.00 | 26.66 |
| ATOM | 5367 | O | PRO | 784 | −18.053 | 2.640 | −11.893 | 1.00 | 27.09 |
| ATOM | 5368 | N | VAL | 785 | −17.509 | 1.788 | −13.906 | 1.00 | 26.20 |
| ATOM | 5369 | CA | VAL | 785 | −17.191 | 3.075 | −14.512 | 1.00 | 26.42 |
| ATOM | 5370 | CB | VAL | 785 | −16.897 | 2.922 | −16.014 | 1.00 | 26.52 |
| ATOM | 5371 | CG1 | VAL | 785 | −16.455 | 4.253 | −16.602 | 1.00 | 27.04 |
| ATOM | 5372 | CG2 | VAL | 785 | −15.821 | 1.878 | −16.221 | 1.00 | 26.53 |
| ATOM | 5373 | C | VAL | 785 | −18.287 | 4.111 | −14.343 | 1.00 | 26.76 |
| ATOM | 5374 | O | VAL | 765 | −18.021 | 5.244 | −13.939 | 1.00 | 23.64 |
| ATOM | 5375 | N | GLU | 786 | −19.519 | 3.728 | −14.664 | 1.00 | 26.86 |
| ATOM | 5376 | CA | GLU | 786 | −20.639 | 4.648 | −14.542 | 1.00 | 26.47 |
| ATOM | 5377 | CB | GLU | 786 | −21.953 | 3.966 | −14.959 | 1.00 | 29.51 |
| ATOM | 5378 | CG | GLU | 786 | −21.954 | 2.441 | −14.909 | 1.00 | 33.26 |
| ATOM | 5379 | CD | GLU | 786 | −21.024 | 1.816 | −15.941 | 1.00 | 33.35 |
| ATOM | 5380 | OE1 | GLU | 786 | −21.069 | 2.234 | −17.115 | 1.00 | 35.72 |
| ATOM | 5381 | OE2 | GLU | 786 | −20.257 | 0.900 | −15.578 | 1.00 | 36.70 |
| ATOM | 5382 | C | GLU | 786 | −20.752 | 5.203 | −13.125 | 1.00 | 25.75 |
| ATOM | 5383 | O | GLU | 786 | −21.142 | 6.352 | −12.934 | 1.00 | 25.18 |
| ATOM | 5384 | N | LEU | 787 | −20.400 | 4.396 | −12.130 | 1.00 | 25.85 |
| ATOM | 5385 | CA | LEU | 787 | −20.470 | 4.854 | −10.749 | 1.00 | 24.33 |
| ATOM | 5386 | CB | LEU | 787 | −20.375 | 3.662 | −9.797 | 1.00 | 27.24 |
| ATOM | 5387 | CG | LEU | 787 | −20.739 | 3.896 | −8.330 | 1.00 | 29.42 |
| ATOM | 5388 | CD1 | LEU | 787 | −20.937 | 2.553 | −7.652 | 1.00 | 30.34 |
| ATOM | 5389 | CD2 | LEU | 787 | −19.666 | 4.691 | −7.632 | 1.00 | 30.31 |
| ATOM | 5390 | C | LEU | 787 | −19.338 | 5.846 | −10.481 | 1.00 | 24.18 |
| ATOM | 5391 | O | LEU | 787 | −19.519 | 6.834 | −9.764 | 1.00 | 21.41 |
| ATOM | 5392 | N | ALA | 788 | −18.171 | 5.579 | −11.062 | 1.00 | 23.68 |
| ATOM | 5393 | CA | ALA | 788 | −17.019 | 6.456 | −10.895 | 1.00 | 23.32 |
| ATOM | 5394 | CB | ALA | 788 | −15.794 | 5.843 | −11.564 | 1.00 | 21.55 |
| ATOM | 5395 | C | ALA | 788 | −17.337 | 7.817 | −11.510 | 1.00 | 23.99 |
| ATOM | 5396 | O | ALA | 788 | −16.890 | 8.855 | −11.020 | 1.00 | 23.15 |
| ATOM | 5397 | N | LYS | 789 | −18.117 | 7.808 | −12.586 | 1.00 | 25.42 |
| ATOM | 5398 | CA | LYS | 789 | −18.495 | 9.050 | −13.254 | 1.00 | 25.60 |
| ATOM | 5399 | CB | LYS | 789 | −19.273 | 8.758 | −14.537 | 1.00 | 29.58 |
| ATOM | 5400 | CG | LYS | 789 | −18.679 | 7.660 | −15.392 | 1.00 | 34.03 |
| ATOM | 5401 | CD | LYS | 789 | −19.410 | 7.537 | −16.722 | 1.00 | 37.45 |
| ATOM | 5402 | CE | LYS | 789 | −18.909 | 6.342 | −17.513 | 1.00 | 37.98 |
| ATOM | 5403 | NZ | LYS | 789 | −19.397 | 6.370 | −18.912 | 1.00 | 39.55 |
| ATOM | 5404 | C | LYS | 789 | −19.365 | 9.883 | −12.323 | 1.00 | 24.36 |
| ATOM | 5405 | O | LYS | 789 | −19.124 | 11.074 | −12.140 | 1.00 | 23.55 |
| ATOM | 5406 | N | ARG | 790 | −20.380 | 9.254 | −11.737 | 1.00 | 23.75 |
| ATOM | 5407 | CA | ARG | 790 | −21.275 | 9.959 | −10.824 | 1.00 | 23.75 |
| ATOM | 5408 | CB | ARG | 790 | −22.320 | 9.011 | −10.229 | 1.00 | 26.20 |
| ATOM | 5409 | CG | ARG | 790 | −23.400 | 8.564 | −11.192 | 1.00 | 31.43 |
| ATOM | 5410 | CD | ARG | 790 | −24.747 | 8.502 | −10.483 | 1.00 | 34.23 |
| ATOM | 5411 | NE | ARG | 790 | −24.766 | 7.564 | −9.362 | 1.00 | 33.07 |
| ATOM | 5412 | CZ | ARG | 790 | −25.553 | 7.694 | −8.297 | 1.00 | 35.30 |
| ATOM | 5413 | NH1 | ARG | 790 | −26.380 | 8.725 | −8.205 | 1.00 | 37.31 |
| ATOM | 5414 | NH2 | ARG | 790 | −25.525 | 6.793 | −7.323 | 1.00 | 35.56 |
| ATOM | 5415 | C | ARG | 790 | −20.510 | 10.612 | −9.680 | 1.00 | 22.42 |
| ATOM | 5416 | O | ARG | 790 | −20.752 | 11.767 | −9.329 | 1.00 | 20.30 |
| ATOM | 5417 | N | ILE | 791 | −19.584 | 9.864 | −9.098 | 1.00 | 20.17 |
| ATOM | 5418 | CA | ILE | 791 | −18.811 | 10.374 | −7.979 | 1.00 | 18.67 |
| ATOM | 5419 | CB | ILE | 791 | −17.999 | 9.232 | −7.310 | 1.00 | 18.12 |
| ATOM | 5420 | CG2 | ILE | 791 | −16.996 | 9.802 | −6.317 | 1.00 | 16.97 |
| ATOM | 5421 | CG1 | ILE | 791 | −18.971 | 8.276 | −6.599 | 1.00 | 20.08 |
| ATOM | 5422 | CD1 | ILE | 791 | −18.299 | 7.133 | −5.849 | 1.00 | 23.20 |
| ATOM | 5423 | C | ILE | 791 | −17.883 | 11.504 | −8.402 | 1.00 | 15.86 |
| ATOM | 5424 | O | ILE | 791 | −17.806 | 12.533 | −7.732 | 1.00 | 15.97 |
| ATOM | 5425 | N | THR | 792 | −17.192 | 11.315 | −9.519 | 1.00 | 17.74 |
| ATOM | 5426 | CA | THR | 792 | −16.271 | 12.326 | −10.014 | 1.00 | 19.29 |
| ATOM | 5427 | CB | THR | 792 | −15.511 | 11.819 | −11.242 | 1.00 | 19.17 |
| ATOM | 5428 | OG1 | THR | 792 | −14.779 | 10.636 | −10.890 | 1.00 | 17.58 |
| ATOM | 5429 | CG2 | THR | 792 | −14.539 | 12.879 | −11.737 | 1.00 | 18.00 |
| ATOM | 5430 | C | THR | 792 | −17.004 | 13.622 | −10.360 | 1.00 | 20.60 |
| ATOM | 5431 | O | THR | 792 | −16.476 | 14.712 | −10.145 | 1.00 | 20.78 |
| ATOM | 5432 | N | GLU | 793 | −18.216 | 13.509 | −10.891 | 1.00 | 20.13 |
| ATOM | 5433 | CA | GLU | 793 | −18.971 | 14.706 | −11.236 | 1.00 | 21.99 |
| ATOM | 5434 | CB | GLU | 793 | −20.014 | 14.392 | −12.307 | 1.00 | 24.27 |
| ATOM | 5435 | CG | GLU | 793 | −19.412 | 13.848 | −13.589 | 1.00 | 27.80 |
| ATOM | 5436 | CD | GLU | 793 | −20.427 | 13.745 | −14.703 | 1.00 | 31.99 |
| ATOM | 5437 | OE1 | GLU | 793 | −21.513 | 13.160 | −14.477 | 1.00 | 33.12 |
| ATOM | 5438 | OE2 | GLU | 793 | −20.135 | 14.243 | −15.808 | 1.00 | 34.84 |
| ATOM | 5439 | C | GLU | 793 | −19.654 | 15.337 | −10.023 | 1.00 | 21.66 |
| ATOM | 5440 | O | GLU | 793 | −19.869 | 16.546 | −9.990 | 1.00 | 22.52 |
| ATOM | 5441 | N | ALA | 794 | −19.990 | 14.528 | −9.023 | 1.00 | 20.04 |
| ATOM | 5442 | CA | ALA | 794 | −20.648 | 15.054 | −7.832 | 1.00 | 19.93 |
| ATOM | 5443 | CB | ALA | 794 | −21.394 | 13.936 | −7.102 | 1.00 | 21.36 |
| ATOM | 5444 | C | ALA | 794 | −19.679 | 15.739 | −6.868 | 1.00 | 20.03 |
| ATOM | 5445 | O | ALA | 794 | −20.088 | 16.572 | −6.064 | 1.00 | 20.59 |
| ATOM | 5446 | N | LEU | 795 | −18.399 | 15.396 | −6.940 | 1.00 | 19.04 |
| ATOM | 5447 | CA | LEU | 795 | −17.424 | 15.990 | −6.034 | 1.00 | 17.59 |
| ATOM | 5448 | CB | LEU | 795 | −16.488 | 14.910 | −5.483 | 1.00 | 20.28 |
| ATOM | 5449 | CG | LEU | 795 | −17.098 | 13.760 | −4.674 | 1.00 | 23.20 |
| ATOM | 5450 | CD1 | LEU | 795 | −15.962 | 12.889 | −4.142 | 1.00 | 22.33 |
| ATOM | 5451 | CD2 | LEU | 795 | −17.934 | 14.285 | −3.527 | 1.00 | 26.37 |
| ATOM | 5452 | C | LEU | 795 | −16.586 | 17.098 | −6.657 | 1.00 | 15.79 |
| ATOM | 5453 | O | LEU | 795 | −16.268 | 17.065 | −7.846 | 1.00 | 16.54 |
| ATOM | 5454 | N | ALA | 796 | −16.227 | 18.082 | −5.840 | 1.00 | 15.08 |
| ATOM | 5455 | CA | ALA | 796 | −15.404 | 19.185 | −6.310 | 1.00 | 15.70 |
| ATOM | 5456 | CB | ALA | 796 | −15.684 | 20.435 | −5.494 | 1.00 | 15.40 |
| ATOM | 5457 | C | ALA | 796 | −13.936 | 18.789 | −6.193 | 1.00 | 14.51 |
| ATOM | 5458 | O | ALA | 796 | −13.108 | 19.193 | −7.008 | 1.00 | 13.29 |
| ATOM | 5459 | N | ILE | 797 | −13.617 | 17.989 | −5.175 | 1.00 | 14.50 |
| ATOM | 5460 | CA | ILE | 797 | −12.243 | 17.546 | −4.972 | 1.00 | 13.86 |
| ATOM | 5461 | CB | ILE | 797 | −12.029 | 16.916 | −3.560 | 1.00 | 14.10 |
| ATOM | 5462 | CG2 | ILE | 797 | −12.315 | 17.932 | −2.488 | 1.00 | 13.02 |
| ATOM | 5463 | CG1 | ILE | 797 | −12.932 | 15.694 | −3.365 | 1.00 | 15.07 |
| ATOM | 5464 | CD1 | ILE | 797 | −12.592 | 14.909 | −2.090 | 1.00 | 16.44 |
| ATOM | 5465 | C | ILE | 797 | −11.862 | 16.526 | −6.034 | 1.00 | 12.69 |
| ATOM | 5466 | O | ILE | 797 | −12.720 | 15.843 | −6.595 | 1.00 | 14.87 |
| ATOM | 5467 | N | PRO | 798 | −10.565 | 16.419 | −6.340 | 1.00 | 14.26 |
| ATOM | 5468 | CD | PRO | 798 | −9.444 | 17.299 | −5.974 | 1.00 | 15.87 |
| ATOM | 5469 | CA | PRO | 798 | −10.181 | 15.446 | −7.359 | 1.00 | 13.35 |
| ATOM | 5470 | CB | PRO | 798 | −8.720 | 15.796 | −7.660 | 1.00 | 15.01 |
| ATOM | 5471 | CG | PRO | 798 | −8.256 | 16.532 | −6.455 | 1.00 | 18.28 |
| ATOM | 5472 | C | PRO | 798 | −10.377 | 14.004 | −6.920 | 1.00 | 13.85 |
| ATOM | 5473 | O | PRO | 798 | −10.202 | 13.665 | −5.745 | 1.00 | 12.45 |
| ATOM | 5474 | N | VAL | 799 | −10.764 | 13.169 | −7.879 | 1.00 | 13.99 |
| ATOM | 5475 | CA | VAL | 799 | −10.992 | 11.750 | −7.633 | 1.00 | 15.13 |
| ATOM | 5476 | CB | VAL | 799 | −12.432 | 11.349 | −8.019 | 1.00 | 16.02 |
| ATOM | 5477 | CG1 | VAL | 799 | −12.605 | 9.845 | −7.864 | 1.00 | 14.52 |
| ATOM | 5478 | CG2 | VAL | 799 | −13.422 | 12.080 | −7.119 | 1.00 | 14.06 |
| ATOM | 5479 | C | VAL | 799 | −9.994 | 10.941 | −8.439 | 1.00 | 14.87 |
| ATOM | 5480 | O | VAL | 799 | −9.949 | 11.029 | −9.663 | 1.00 | 15.59 |
| ATOM | 5481 | N | ILE | 800 | −9.186 | 10.162 | −7.731 | 1.00 | 14.30 |
| ATOM | 5482 | CA | ILE | 800 | −8.170 | 9.335 | −8.350 | 1.00 | 14.45 |
| ATOM | 5483 | CB | ILE | 800 | −6.861 | 9.375 | −7.528 | 1.00 | 15.69 |
| ATOM | 5484 | CG2 | ILE | 800 | −5.830 | 8.414 | −8.128 | 1.00 | 14.51 |
| ATOM | 5485 | CG1 | ILE | 800 | −6.315 | 10.810 | −7.524 | 1.00 | 15.10 |
| ATOM | 5486 | CD1 | ILE | 800 | −5.074 | 11.021 | −6.676 | 1.00 | 17.30 |
| ATOM | 5487 | C | ILE | 800 | −8.691 | 7.911 | −8.429 | 1.00 | 14.61 |
| ATOM | 5488 | O | ILE | 800 | −9.113 | 7.334 | −7.424 | 1.00 | 13.66 |
| ATOM | 5489 | N | GLY | 801 | −8.672 | 7.349 | −9.629 | 1.00 | 13.09 |
| ATOM | 5490 | CA | GLY | 801 | −9.178 | 6.005 | −9.783 | 1.00 | 12.12 |
| ATOM | 5491 | C | GLY | 801 | −8.151 | 4.935 | −10.061 | 1.00 | 13.23 |
| ATOM | 5492 | O | GLY | 801 | −7.055 | 5.200 | −10.549 | 1.00 | 12.71 |
| ATOM | 5493 | N | ILE | 802 | −8.522 | 3.713 | −9.703 | 1.00 | 14.81 |
| ATOM | 5494 | CA | ILE | 802 | −7.712 | 2.533 | −9.954 | 1.00 | 16.00 |
| ATOM | 5495 | CB | ILE | 802 | −6.799 | 2.164 | −8.739 | 1.00 | 15.76 |
| ATOM | 5496 | CG2 | ILE | 802 | −7.575 | 2.202 | −7.439 | 1.00 | 18.40 |
| ATOM | 5497 | CG1 | ILE | 802 | −6.172 | 0.792 | −8.967 | 1.00 | 20.03 |
| ATOM | 5498 | CD1 | ILE | 802 | −5.323 | 0.706 | −10.195 | 1.00 | 20.86 |
| ATOM | 5499 | C | ILE | 802 | −8.773 | 1.468 | −10.223 | 1.00 | 15.80 |
| ATOM | 5500 | O | ILE | 802 | −9.548 | 1.112 | −9.343 | 1.00 | 16.52 |
| ATOM | 5501 | N | GLY | 803 | −8.831 | 1.008 | −11.466 | 1.00 | 18.14 |
| ATOM | 5502 | CA | GLY | 803 | −9.826 | 0.024 | −11.834 | 1.00 | 18.81 |
| ATOM | 5503 | C | GLY | 803 | −11.211 | 0.649 | −11.819 | 1.00 | 19.32 |
| ATOM | 5504 | O | GLY | 803 | −12.206 | −0.041 | −11.600 | 1.00 | 20.70 |
| ATOM | 5505 | N | ALA | 804 | −11.278 | 1.957 | −12.049 | 1.00 | 17.72 |
| ATOM | 5506 | CA | ALA | 804 | −12.544 | 2.676 | −12.048 | 1.00 | 18.13 |
| ATOM | 5507 | CB | ALA | 804 | −12.547 | 3.719 | −10.922 | 1.00 | 16.05 |
| ATOM | 5508 | C | ALA | 804 | −12.834 | 3.359 | −13.382 | 1.00 | 17.22 |
| ATOM | 5509 | O | ALA | 804 | −13.727 | 4.205 | −13.478 | 1.00 | 19.29 |
| ATOM | 5510 | N | GLY | 805 | −12.079 | 2.999 | −14.412 | 1.00 | 17.74 |
| ATOM | 5511 | CA | GLY | 805 | −12.289 | 3.595 | −15.718 | 1.00 | 19.47 |
| ATOM | 5512 | C | GLY | 805 | −11.524 | 4.893 | −15.885 | 1.00 | 19.16 |
| ATOM | 5513 | O | GLY | 805 | −10.832 | 5.336 | −14.964 | 1.00 | 19.03 |
| ATOM | 5514 | N | ASN | 806 | −11.646 | 5.509 | −17.057 | 1.00 | 17.88 |
| ATOM | 5515 | CA | ASN | 806 | −10.939 | 6.760 | −17.324 | 1.00 | 18.23 |
| ATOM | 5516 | CB | ASN | 806 | −10.410 | 6.784 | −18.769 | 1.00 | 16.49 |
| ATOM | 5517 | CG | ASN | 806 | −11.511 | 6.953 | −19.810 | 1.00 | 18.05 |
| ATOM | 5518 | OD1 | ASN | 806 | −11.225 | 7.247 | −20.972 | 1.00 | 19.59 |
| ATOM | 5519 | ND2 | ASN | 806 | −12.767 | 6.760 | −19.404 | 1.00 | 12.05 |
| ATOM | 5520 | C | ASN | 806 | −11.774 | 8.006 | −17.062 | 1.00 | 18.00 |
| ATOM | 5521 | O | ASN | 806 | −11.411 | 9.091 | −17.497 | 1.00 | 18.99 |
| ATOM | 5522 | N | VAL | 807 | −12.872 | 7.848 | −16.331 | 1.00 | 17.63 |
| ATOM | 5523 | CA | VAL | 807 | −13.756 | 8.965 | −16.033 | 1.00 | 19.39 |
| ATOM | 5524 | CB | VAL | 807 | −15.205 | 8.489 | −15.860 | 1.00 | 21.52 |
| ATOM | 5525 | CG1 | VAL | 807 | −16.140 | 9.685 | −15.847 | 1.00 | 27.73 |
| ATOM | 5526 | CG2 | VAL | 807 | −15.578 | 7.547 | −16.990 | 1.00 | 24.38 |
| ATOM | 5527 | C | VAL | 807 | −13.354 | 9.738 | −14.783 | 1.00 | 18.27 |
| ATOM | 5528 | O | VAL | 807 | −13.941 | 10.770 | −14.478 | 1.00 | 16.18 |
| ATOM | 5529 | N | THR | 808 | −12.361 | 9.237 | −14.057 | 1.00 | 18.22 |
| ATOM | 5530 | CA | THR | 808 | −11.899 | 9.915 | −12.854 | 1.00 | 14.60 |
| ATOM | 5531 | CB | THR | 808 | −11.203 | 8.920 | −11.885 | 1.00 | 16.31 |
| ATOM | 5532 | OG1 | THR | 808 | −10.153 | 8.222 | −12.567 | 1.00 | 14.17 |
| ATOM | 5533 | CG2 | THR | 808 | −12.215 | 7.923 | −11.346 | 1.00 | 12.82 |
| ATOM | 5534 | C | THR | 808 | −10.944 | 11.044 | −13.250 | 1.00 | 16.13 |
| ATOM | 5535 | O | THR | 808 | −10.476 | 11.103 | −14.390 | 1.00 | 16.23 |
| ATOM | 5536 | N | ASP | 809 | −10.675 | 11.948 | −12.312 | 1.00 | 15.76 |
| ATOM | 5537 | CA | ASP | 809 | −9.790 | 13.086 | −12.556 | 1.00 | 15.32 |
| ATOM | 5538 | CB | ASP | 809 | −9.912 | 14.095 | −11.405 | 1.00 | 14.96 |
| ATOM | 5539 | CG | ASP | 809 | −11.332 | 14.591 | −11.221 | 1.00 | 15.54 |
| ATOM | 5540 | OD1 | ASP | 809 | −11.890 | 15.153 | −12.191 | 1.00 | 17.46 |
| ATOM | 5541 | OD2 | ASP | 809 | −11.897 | 14.428 | −10.126 | 1.00 | 17.01 |
| ATOM | 5542 | C | ASP | 809 | −8.342 | 12.633 | −12.691 | 1.00 | 15.52 |
| ATOM | 5543 | O | ASP | 809 | −7.535 | 13.266 | −13.379 | 1.00 | 14.60 |
| ATOM | 5544 | N | GLY | 810 | −8.021 | 11.528 | −12.031 | 1.00 | 15.18 |
| ATOM | 5545 | CA | GLY | 810 | −6.673 | 11.011 | −12.089 | 1.00 | 15.09 |
| ATOM | 5546 | C | GLY | 810 | −6.630 | 9.501 | −12.037 | 1.00 | 14.54 |
| ATOM | 5547 | O | GLY | 810 | −7.656 | 8.831 | −11.914 | 1.00 | 14.65 |
| ATOM | 5548 | N | GLN | 811 | −5.424 | 8.964 | −12.115 | 1.00 | 14.84 |
| ATOM | 5549 | CA | GLN | 811 | −5.242 | 7.528 | −12.093 | 1.00 | 14.60 |
| ATOM | 5550 | CB | GLN | 811 | −4.929 | 7.035 | −13.506 | 1.00 | 15.54 |
| ATOM | 5551 | CG | GLN | 811 | −6.026 | 7.268 | −14.523 | 1.00 | 16.05 |
| ATOM | 5552 | CD | GLN | 811 | −7.254 | 6.423 | −14.248 | 1.00 | 14.86 |
| ATOM | 5553 | OE1 | GLN | 811 | −7.145 | 5.274 | −13.812 | 1.00 | 15.67 |
| ATOM | 5554 | NE2 | GLN | 811 | −8.428 | 6.982 | −14.513 | 1.00 | 13.00 |
| ATOM | 5555 | C | GLN | 811 | −4.095 | 7.146 | −11.173 | 1.00 | 16.44 |
| ATOM | 5556 | O | GLN | 811 | −3.179 | 7.933 | −10.940 | 1.00 | 12.57 |
| ATOM | 5557 | N | ILE | 812 | −4.151 | 5.932 | −10.648 | 1.00 | 18.01 |
| ATOM | 5558 | CA | ILE | 812 | −3.080 | 5.448 | −9.795 | 1.00 | 21.55 |
| ATOM | 5559 | CB | ILE | 812 | −3.394 | 5.680 | −8.286 | 1.00 | 22.97 |
| ATOM | 5560 | CG2 | ILE | 812 | −4.477 | 4.732 | −7.810 | 1.00 | 24.22 |
| ATOM | 5561 | CG1 | ILE | 812 | −2.116 | 5.495 | −7.461 | 1.00 | 26.66 |
| ATOM | 5562 | CD1 | ILE | 812 | −2.168 | 6.141 | −6.077 | 1.00 | 27.37 |
| ATOM | 5563 | C | ILE | 812 | −2.880 | 3.968 | −10.098 | 1.00 | 23.39 |
| ATOM | 5564 | O | ILE | 812 | −3.781 | 3.292 | −10.599 | 1.00 | 22.15 |
| ATOM | 5565 | N | LEU | 813 | −1.678 | 3.478 | −9.832 | 1.00 | 25.42 |
| ATOM | 5566 | CA | LEU | 813 | −1.369 | 2.081 | −10.072 | 1.00 | 27.98 |
| ATOM | 5567 | CB | LEU | 813 | −1.231 | 1.814 | −11.572 | 1.00 | 29.46 |
| ATOM | 5568 | CG | LEU | 813 | −2.228 | 0.842 | −12.220 | 1.00 | 33.93 |
| ATOM | 5569 | CD1 | LEU | 813 | −1.759 | 0.552 | −13.636 | 1.00 | 36.02 |
| ATOM | 5570 | CD2 | LEU | 813 | −2.306 | −0.460 | −11.426 | 1.00 | 34.60 |
| ATOM | 5571 | C | LEU | 813 | −0.075 | 1.704 | −9.375 | 1.00 | 27.36 |
| ATOM | 5572 | O | LEU | 813 | 0.783 | 2.558 | −9.138 | 1.00 | 24.70 |
| ATOM | 5573 | N | VAL | 814 | 0.046 | 0.422 | −9.038 | 1.00 | 27.73 |
| ATOM | 5574 | CA | VAL | 814 | 1.238 | −0.099 | −8.388 | 1.00 | 24.77 |
| ATOM | 5575 | CB | VAL | 814 | 0.992 | −1.504 | −7.775 | 1.00 | 26.98 |
| ATOM | 5576 | CG1 | VAL | 814 | 2.285 | −2.045 | −7.170 | 1.00 | 27.58 |
| ATOM | 5577 | CG2 | VAL | 814 | −0.088 | −1.420 | −6.705 | 1.00 | 29.29 |
| ATOM | 5578 | C | VAL | 814 | 2.313 | −0.208 | −9.456 | 1.00 | 21.90 |
| ATOM | 5579 | O | VAL | 814 | 2.128 | −0.851 | −10.487 | 1.00 | 19.33 |
| ATOM | 5580 | N | MET | 815 | 3.441 | 0.438 | −9.202 | 1.00 | 20.34 |
| ATOM | 5581 | CA | MET | 815 | 4.546 | 0.432 | −10.142 | 1.00 | 18.61 |
| ATOM | 5582 | CB | MET | 815 | 5.710 | 1.244 | −9.564 | 1.00 | 15.22 |
| ATOM | 5583 | CG | MET | 815 | 6.183 | 0.786 | −8.190 | 1.00 | 15.35 |
| ATOM | 5584 | SD | MET | 815 | 7.964 | 0.942 | −8.143 | 1.00 | 16.36 |
| ATOM | 5585 | CE | MET | 815 | 8.452 | −0.581 | −8.957 | 1.00 | 11.50 |
| ATOM | 5586 | C | MET | 815 | 5.020 | −0.973 | −10.521 | 1.00 | 17.74 |
| ATOM | 5587 | O | MET | 815 | 5.483 | −1.205 | −11.639 | 1.00 | 19.67 |
| ATOM | 5588 | N | HIS | 816 | 4.908 | −1.918 | −9.597 | 1.00 | 17.17 |
| ATOM | 5589 | CA | HIS | 816 | 5.358 | −3.276 | −9.870 | 1.00 | 17.66 |
| ATOM | 5590 | CB | HIS | 816 | 5.319 | −4.091 | −8.570 | 1.00 | 16.96 |
| ATOM | 5591 | CG | HIS | 816 | 6.334 | −3.639 | −7.567 | 1.00 | 16.58 |
| ATOM | 5592 | CD2 | HIS | 816 | 6.312 | −2.620 | −6.675 | 1.00 | 14.97 |
| ATOM | 5593 | ND1 | HIS | 816 | 7.604 | −4.173 | −7.503 | 1.00 | 18.92 |
| ATOM | 5594 | CE1 | HIS | 816 | 8.322 | −3.500 | −6.622 | 1.00 | 15.55 |
| ATOM | 5595 | NE2 | HIS | 816 | 7.561 | −2.551 | −6.106 | 1.00 | 21.47 |
| ATOM | 5596 | C | HIS | 816 | 4.578 | −3.953 | −10.994 | 1.00 | 20.90 |
| ATOM | 5597 | O | HIS | 816 | 5.096 | −4.844 | −11.663 | 1.00 | 20.05 |
| ATOM | 5598 | N | ASP | 817 | 3.340 | −3.524 | −11.217 | 1.00 | 23.00 |
| ATOM | 5599 | CA | ASP | 817 | 2.526 | −4.095 | −12.294 | 1.00 | 25.53 |
| ATOM | 5600 | CB | ASP | 817 | 1.041 | −4.013 | −11.939 | 1.00 | 29.00 |
| ATOM | 5601 | CG | ASP | 817 | 0.689 | −4.830 | −10.710 | 1.00 | 32.54 |
| ATOM | 5602 | OD1 | ASP | 817 | 0.878 | −6.062 | −10.742 | 1.00 | 34.60 |
| ATOM | 5603 | OD2 | ASP | 817 | 0.224 | −4.234 | −9.716 | 1.00 | 34.24 |
| ATOM | 5604 | C | ASP | 817 | 2.786 | −3.341 | −13.602 | 1.00 | 26.31 |
| ATOM | 5605 | O | ASP | 817 | 2.743 | −3.925 | −14.687 | 1.00 | 27.79 |
| ATOM | 5606 | N | ALA | 818 | 3.064 | −2.045 | −13.489 | 1.00 | 27.64 |
| ATOM | 5607 | CA | ALA | 818 | 3.330 | −1.195 | −14.651 | 1.00 | 27.51 |
| ATOM | 5608 | CB | ALA | 818 | 3.461 | 0.262 | −14.204 | 1.00 | 28.70 |
| ATOM | 5609 | C | ALA | 818 | 4.573 | −1.609 | −15.438 | 1.00 | 28.22 |
| ATOM | 5610 | O | ALA | 818 | 4.661 | −1.348 | −16.640 | 1.00 | 27.43 |
| ATOM | 5611 | N | PHE | 819 | 5.536 | −2.242 | −14.765 | 1.00 | 26.40 |
| ATOM | 5612 | CA | PHE | 819 | 6.764 | −2.676 | −15.434 | 1.00 | 25.70 |
| ATOM | 5613 | CB | PHE | 819 | 7.994 | −2.133 | −14.705 | 1.00 | 26.29 |
| ATOM | 5614 | CG | PHE | 819 | 7.889 | −0.677 | −14.359 | 1.00 | 27.48 |
| ATOM | 5615 | CD1 | PHE | 819 | 7.412 | 0.237 | −15.292 | 1.00 | 28.59 |
| ATOM | 5616 | CD2 | PHE | 819 | 8.248 | −0.219 | −13.094 | 1.00 | 28.81 |
| ATOM | 5617 | CE1 | PHE | 819 | 7.289 | 1.588 | −14.975 | 1.00 | 29.43 |
| ATOM | 5618 | CE2 | PHE | 819 | 8.131 | 1.132 | −12.763 | 1.00 | 29.98 |
| ATOM | 5619 | CZ | PHE | 819 | 7.648 | 2.037 | −13.707 | 1.00 | 30.70 |
| ATOM | 5620 | C | PHE | 819 | 6.869 | −4.191 | −15.551 | 1.00 | 25.12 |
| ATOM | 5621 | O | PHE | 819 | 7.935 | −4.733 | −15.846 | 1.00 | 24.68 |
| ATOM | 5622 | N | GLY | 820 | 5.753 | −4.872 | −15.323 | 1.00 | 24.44 |
| ATOM | 5623 | CA | GLY | 820 | 5.738 | −6.320 | −15.419 | 1.00 | 24.57 |
| ATOM | 5624 | C | GLY | 820 | 6.655 | −7.010 | −14.429 | 1.00 | 25.69 |
| ATOM | 5625 | O | GLY | 820 | 7.124 | −8.120 | −14.688 | 1.00 | 25.84 |
| ATOM | 5626 | N | ILE | 821 | 6.919 | −6.360 | −13.297 | 1.00 | 23.27 |
| ATOM | 5627 | CA | ILE | 821 | 7.779 | −6.952 | −12.277 | 1.00 | 22.14 |
| ATOM | 5628 | CB | ILE | 821 | 8.241 | −5.891 | −11.247 | 1.00 | 20.40 |
| ATOM | 5629 | CG2 | ILE | 821 | 9.023 | −6.565 | −10.110 | 1.00 | 18.38 |
| ATOM | 5630 | CG1 | ILE | 821 | 9.099 | −4.839 | −11.956 | 1.00 | 19.76 |
| ATOM | 5631 | CD1 | ILE | 821 | 9.420 | −3.621 | −11.125 | 1.00 | 17.39 |
| ATOM | 5632 | C | ILE | 821 | 7.015 | −8.065 | −11.565 | 1.00 | 22.87 |
| ATOM | 5633 | O | ILE | 821 | 7.542 | −9.153 | −11.342 | 1.00 | 21.66 |
| ATOM | 5634 | N | THR | 822 | 5.757 | −7.788 | −11.238 | 1.00 | 24.23 |
| ATOM | 5635 | CA | THR | 822 | 4.916 | −8.761 | −10.562 | 1.00 | 28.76 |
| ATOM | 5636 | CB | THR | 822 | 3.548 | −8.147 | −10.196 | 1.00 | 29.81 |
| ATOM | 5637 | OG1 | THR | 822 | 3.020 | −7.441 | −11.327 | 1.00 | 35.04 |
| ATOM | 5638 | CG2 | THR | 822 | 3.697 | −7.184 | −9.036 | 1.00 | 29.52 |
| ATOM | 5639 | C | THR | 822 | 4.689 | −10.014 | −11.407 | 1.00 | 31.12 |
| ATOM | 5640 | O | THR | 822 | 4.675 | −9.948 | −12.638 | 1.00 | 28.97 |
| ATOM | 5641 | N | GLY | 823 | 4.514 | −11.142 | −10.718 | 1.00 | 33.39 |
| ATOM | 5642 | CA | GLY | 823 | 4.285 | −12.431 | −11.354 | 1.00 | 39.65 |
| ATOM | 5643 | C | GLY | 823 | 4.137 | −12.444 | −12.863 | 1.00 | 43.12 |
| ATOM | 5644 | O | GLY | 823 | 5.124 | −12.358 | −13.591 | 1.00 | 45.19 |
| ATOM | 5645 | N | GLY | 824 | 2.903 | −12.560 | −13.338 | 1.00 | 45.52 |
| ATOM | 5646 | CA | GLY | 824 | 2.671 | −12.584 | −14.769 | 1.00 | 47.84 |
| ATOM | 5647 | C | GLY | 824 | 1.199 | −12.505 | −15.103 | 1.00 | 49.56 |
| ATOM | 5648 | O | GLY | 824 | 0.818 | −12.463 | −16.271 | 1.00 | 50.56 |
| ATOM | 5649 | N | HIS | 825 | 0.368 | −12.481 | −14.068 | 1.00 | 50.83 |
| ATOM | 5650 | CA | HIS | 825 | −1.077 | −12.408 | −14.248 | 1.00 | 52.44 |
| ATOM | 5651 | CB | HIS | 825 | −1.737 | −13.633 | −13.610 | 1.00 | 54.90 |
| ATOM | 5652 | CG | HIS | 825 | −1.153 | −14.935 | −14.063 | 1.00 | 57.95 |
| ATOM | 5653 | CD2 | HIS | 825 | −0.491 | −15.900 | −13.381 | 1.00 | 58.90 |
| ATOM | 5654 | ND1 | HIS | 825 | −1.195 | −15.355 | −15.376 | 1.00 | 59.02 |
| ATOM | 5655 | CE1 | HIS | 825 | −0.582 | −16.522 | −15.483 | 1.00 | 59.10 |
| ATOM | 5656 | NE2 | HIS | 825 | −0.146 | −16.874 | −14.287 | 1.00 | 58.90 |
| ATOM | 5657 | C | HIS | 825 | −1.625 | −11.131 | −13.613 | 1.00 | 51.18 |
| ATOM | 5658 | O | HIS | 825 | −2.490 | −11.188 | −12.738 | 1.00 | 51.71 |
| ATOM | 5659 | N | ILE | 826 | −1.120 | −9.982 | −14.055 | 1.00 | 50.05 |
| ATOM | 5660 | CA | ILE | 826 | −1.566 | −8.698 | −13.516 | 1.00 | 48.19 |
| ATOM | 5661 | CB | ILE | 826 | −0.883 | −7.510 | −14.230 | 1.00 | 48.89 |
| ATOM | 5662 | CG2 | ILE | 826 | 0.631 | −7.612 | −14.078 | 1.00 | 50.27 |
| ATOM | 5663 | CG1 | ILE | 826 | −1.280 | −7.484 | −15.707 | 1.00 | 49.07 |
| ATOM | 5664 | CD1 | ILE | 826 | −0.762 | −6.273 | −16.460 | 1.00 | 47.09 |
| ATOM | 5665 | C | ILE | 826 | −3.077 | −8.531 | −13.640 | 1.00 | 46.15 |
| ATOM | 5666 | O | ILE | 826 | −3.707 | −9.119 | −14.520 | 1.00 | 45.71 |
| ATOM | 5667 | N | PRO | 827 | −3.678 | −7.719 | −12.758 | 1.00 | 44.37 |
| ATOM | 5668 | CD | PRO | 827 | −3.037 | −6.920 | −11.700 | 1.00 | 43.85 |
| ATOM | 5669 | CA | PRO | 827 | −5.124 | −7.485 | −12.780 | 1.00 | 43.13 |
| ATOM | 5670 | CB | PRO | 827 | −5.344 | −6.592 | −11.560 | 1.00 | 44.15 |
| ATOM | 5671 | CG | PRO | 827 | −4.058 | −5.841 | −11.457 | 1.00 | 45.19 |
| ATOM | 5672 | C | PRO | 827 | −5.625 | −6.849 | −14.073 | 1.00 | 40.91 |
| ATOM | 5673 | O | PRO | 827 | −4.887 | −6.137 | −14.753 | 1.00 | 40.25 |
| ATOM | 5674 | N | LYS | 828 | −6.884 | −7.116 | −14.402 | 1.00 | 39.31 |
| ATOM | 5675 | CA | LYS | 828 | −7.500 | −6.580 | −15.610 | 1.00 | 37.09 |
| ATOM | 5676 | CB | LYS | 828 | −8.976 | −6.986 | −15.682 | 1.00 | 40.38 |
| ATOM | 5677 | CG | LYS | 828 | −9.236 | −8.338 | −16.326 | 1.00 | 45.06 |
| ATOM | 5678 | CD | LYS | 828 | −8.689 | −9.480 | −15.488 | 1.00 | 46.97 |
| ATOM | 5679 | CE | LYS | 828 | −9.021 | −10.818 | −16.122 | 1.00 | 47.07 |
| ATOM | 5680 | NZ | LYS | 828 | −8.544 | −11.962 | −15.299 | 1.00 | 48.51 |
| ATOM | 5681 | C | LYS | 828 | −7.405 | −5.069 | −15.737 | 1.00 | 32.78 |
| ATOM | 5682 | O | LYS | 828 | −7.356 | −4.552 | −16.847 | 1.00 | 32.06 |
| ATOM | 5683 | N | PHE | 829 | −7.385 | −4.364 | −14.608 | 1.00 | 29.83 |
| ATOM | 5684 | CA | PHE | 829 | −7.326 | −2.903 | −14.625 | 1.00 | 26.91 |
| ATOM | 5685 | CB | PHE | 829 | −7.961 | −2.332 | −13.350 | 1.00 | 25.84 |
| ATOM | 5686 | CG | PHE | 829 | −7.302 | −2.794 | −12.077 | 1.00 | 26.76 |
| ATOM | 5687 | CD1 | PHE | 829 | −6.061 | −2.289 | −11.684 | 1.00 | 25.54 |
| ATOM | 5688 | CD2 | PHE | 829 | −7.914 | −3.752 | −11.278 | 1.00 | 25.39 |
| ATOM | 5689 | CE1 | PHE | 829 | −5.447 | −2.739 | −10.514 | 1.00 | 27.51 |
| ATOM | 5690 | CE2 | PHE | 829 | −7.307 | −4.203 | −10.112 | 1.00 | 27.40 |
| ATOM | 5691 | CZ | PHE | 829 | −6.071 | −3.697 | −9.728 | 1.00 | 26.75 |
| ATOM | 5692 | C | PHE | 829 | −5.923 | −2.332 | −14.789 | 1.00 | 26.04 |
| ATOM | 5693 | O | PHE | 829 | −5.766 | −1.136 | −15.010 | 1.00 | 26.02 |
| ATOM | 5694 | N | ALA | 830 | −4.910 | −3.184 | −14.681 | 1.00 | 24.63 |
| ATOM | 5695 | CA | ALA | 830 | −3.529 | −2.736 | −14.813 | 1.00 | 23.28 |
| ATOM | 5696 | CB | ALA | 830 | −2.643 | −3.490 | −13.828 | 1.00 | 21.79 |
| ATOM | 5697 | C | ALA | 830 | −2.997 | −2.914 | −16.229 | 1.00 | 22.75 |
| ATOM | 5698 | O | ALA | 830 | −3.612 | −3.579 | −17.062 | 1.00 | 21.73 |
| ATOM | 5699 | N | LYS | 831 | −1.842 | −2.316 | −16.495 | 1.00 | 21.73 |
| ATOM | 5700 | CA | LYS | 831 | −1.209 | −2.421 | −17.801 | 1.00 | 20.75 |
| ATOM | 5701 | CB | LYS | 831 | −1.694 | −1.305 | −18.731 | 1.00 | 22.17 |
| ATOM | 5702 | CG | LYS | 831 | −0.965 | −1.255 | −20.074 | 1.00 | 22.12 |
| ATOM | 5703 | CD | LYS | 831 | −1.486 | −0.125 | −20.950 | 1.00 | 24.84 |
| ATOM | 5704 | CE | LYS | 831 | −0.669 | 0.000 | −22.227 | 1.00 | 27.33 |
| ATOM | 5705 | NZ | LYS | 831 | −1.132 | 1.104 | −23.117 | 1.00 | 27.97 |
| ATOM | 5706 | C | LYS | 831 | 0.304 | −2.345 | −17.665 | 1.00 | 20.84 |
| ATOM | 5707 | O | LYS | 831 | 0.839 | −1.515 | −16.924 | 1.00 | 20.39 |
| ATOM | 5708 | N | ASN | 832 | 0.985 | −3.226 | −18.387 | 1.00 | 20.11 |
| ATOM | 5709 | CA | ASN | 832 | 2.442 | −3.275 | −18.393 | 1.00 | 20.06 |
| ATOM | 5710 | CB | ASN | 832 | 2.915 | −4.696 | −18.715 | 1.00 | 19.73 |
| ATOM | 5711 | CG | ASN | 832 | 4.430 | −4.820 | −18.755 | 1.00 | 19.21 |
| ATOM | 5712 | OD1 | ASN | 832 | 5.145 | −3.832 | −18.897 | 1.00 | 21.54 |
| ATOM | 5713 | ND2 | ASN | 832 | 4.921 | −6.045 | −18.653 | 1.00 | 18.62 |
| ATOM | 5714 | C | ASN | 832 | 2.933 | −2.323 | −19.480 | 1.00 | 20.14 |
| ATOM | 5715 | O | ASN | 832 | 2.993 | −2.693 | −20.653 | 1.00 | 19.00 |
| ATOM | 5716 | N | PHE | 833 | 3.283 | −1.102 | −19.087 | 1.00 | 20.67 |
| ATOM | 5717 | CA | PHE | 833 | 3.764 | −0.102 | −20.034 | 1.00 | 20.91 |
| ATOM | 5718 | CB | PHE | 833 | 3.696 | 1.301 | −19.418 | 1.00 | 21.37 |
| ATOM | 5719 | CG | PHE | 833 | 2.298 | 1.809 | −19.229 | 1.00 | 21.99 |
| ATOM | 5720 | CD1 | PHE | 833 | 1.581 | 1.505 | −18.075 | 1.00 | 21.53 |
| ATOM | 5721 | CD2 | PHE | 833 | 1.679 | 2.557 | −20.228 | 1.00 | 20.96 |
| ATOM | 5722 | CE1 | PHE | 833 | 0.268 | 1.942 | −17.919 | 1.00 | 23.22 |
| ATOM | 5723 | CE2 | PHE | 833 | 0.367 | 2.997 | −20.081 | 1.00 | 22.83 |
| ATOM | 5724 | CZ | PHE | 833 | −0.345 | 2.689 | −18.926 | 1.00 | 21.37 |
| ATOM | 5725 | C | PHE | 833 | 5.180 | −0.373 | −20.526 | 1.00 | 21.48 |
| ATOM | 5726 | O | PHE | 833 | 5.595 | 0.162 | −21.556 | 1.00 | 22.31 |
| ATOM | 5727 | N | LEU | 834 | 5.919 | −1.201 | −19.793 | 1.00 | 19.73 |
| ATOM | 5728 | CA | LEU | 834 | 7.289 | −1.541 | −20.176 | 1.00 | 20.43 |
| ATOM | 5729 | CB | LEU | 834 | 8.037 | −2.176 | −18.999 | 1.00 | 19.89 |
| ATOM | 5730 | CG | LEU | 834 | 9.493 | −2.566 | −19.278 | 1.00 | 18.42 |
| ATOM | 5731 | CD1 | LEU | 834 | 10.312 | −1.300 | −19.481 | 1.00 | 21.54 |
| ATOM | 5732 | CD2 | LEU | 834 | 10.064 | −3.383 | −18.115 | 1.00 | 20.29 |
| ATOM | 5733 | C | LEU | 834 | 7.301 | −2.513 | −21.348 | 1.00 | 22.45 |
| ATOM | 5734 | O | LEU | 834 | 8.221 | −2.506 | −22.163 | 1.00 | 22.86 |
| ATOM | 5735 | N | ALA | 835 | 6.284 | −3.364 | −21.419 | 1.00 | 25.29 |
| ATOM | 5736 | CA | ALA | 835 | 6.190 | −4.332 | −22.504 | 1.00 | 29.51 |
| ATOM | 5737 | CB | ALA | 835 | 4.968 | −5.221 | −22.310 | 1.00 | 30.38 |
| ATOM | 5738 | C | ALA | 835 | 6.086 | −3.582 | −23.824 | 1.00 | 32.18 |
| ATOM | 5739 | O | ALA | 835 | 6.775 | −3.911 | −24.789 | 1.00 | 32.05 |
| ATOM | 5740 | N | GLU | 836 | 5.207 | −2.581 | −23.843 | 1.00 | 34.79 |
| ATOM | 5741 | CA | GLU | 836 | 4.973 | −1.740 | −25.013 | 1.00 | 38.46 |
| ATOM | 5742 | CB | GLU | 836 | 4.016 | −0.595 | −24.664 | 1.00 | 41.47 |
| ATOM | 5743 | CG | GLU | 836 | 2.611 | −1.015 | −24.239 | 1.00 | 45.34 |
| ATOM | 5744 | CD | GLU | 836 | 1.726 | −1.390 | −25.417 | 1.00 | 47.52 |
| ATOM | 5745 | OE1 | GLU | 836 | 2.091 | −2.328 | −26.160 | 1.00 | 48.57 |
| ATOM | 5746 | OE2 | GLU | 836 | 0.670 | −0.743 | −25.598 | 1.00 | 48.67 |
| ATOM | 5747 | C | GLU | 836 | 6.304 | −1.151 | −25.450 | 1.00 | 38.72 |
| ATOM | 5748 | O | GLU | 836 | 6.690 | −1.249 | −26.612 | 1.00 | 41.32 |
| ATOM | 5749 | N | THR | 837 | 6.997 | −0.524 | −24.506 | 1.00 | 37.17 |
| ATOM | 5750 | CA | THR | 837 | 8.284 | 0.072 | −24.796 | 1.00 | 36.61 |
| ATOM | 5751 | CB | THR | 837 | 8.505 | 1.361 | −23.987 | 1.00 | 36.83 |
| ATOM | 5752 | OG1 | THR | 837 | 9.822 | 1.867 | −24.253 | 1.00 | 38.80 |
| ATOM | 5753 | CG2 | THR | 837 | 8.351 | 1.096 | −22.489 | 1.00 | 35.24 |
| ATOM | 5754 | C | THR | 837 | 9.393 | −0.920 | −24.480 | 1.00 | 36.87 |
| ATOM | 5755 | O | THR | 837 | 9.207 | −2.133 | −24.616 | 1.00 | 38.89 |
| ATOM | 5756 | N | GLY | 838 | 10.546 | −0.407 | −24.067 | 1.00 | 33.88 |
| ATOM | 5757 | CA | GLY | 838 | 11.662 | −1.272 | −23.738 | 1.00 | 31.57 |
| ATOM | 5758 | C | GLY | 838 | 12.514 | −0.674 | −22.640 | 1.00 | 28.89 |
| ATOM | 5759 | O | GLY | 838 | 13.484 | −1.277 | −22.201 | 1.00 | 28.70 |
| ATOM | 5760 | N | ASP | 839 | 12.126 | 0.511 | −22.186 | 1.00 | 28.03 |
| ATOM | 5761 | CA | ASP | 839 | 12.852 | 1.236 | −21.154 | 1.00 | 27.44 |
| ATOM | 5762 | CB | ASP | 839 | 13.574 | 2.410 | −21.834 | 1.00 | 32.86 |
| ATOM | 5763 | CG | ASP | 839 | 14.173 | 3.381 | −20.859 | 1.00 | 35.56 |
| ATOM | 5764 | OD1 | ASP | 839 | 13.475 | 4.350 | −20.483 | 1.00 | 36.98 |
| ATOM | 5765 | OD2 | ASP | 839 | 15.347 | 3.181 | −20.470 | 1.00 | 40.04 |
| ATOM | 5766 | C | ASP | 839 | 11.886 | 1.714 | −20.054 | 1.00 | 24.78 |
| ATOM | 5767 | O | ASP | 839 | 10.805 | 2.223 | −20.349 | 1.00 | 24.31 |
| ATOM | 5768 | N | ILE | 840 | 12.274 | 1.550 | −18.789 | 1.00 | 22.68 |
| ATOM | 5769 | CA | ILE | 840 | 11.418 | 1.952 | −17.671 | 1.00 | 19.45 |
| ATOM | 5770 | CB | ILE | 840 | 12.087 | 1.625 | −16.306 | 1.00 | 17.83 |
| ATOM | 5771 | CG2 | ILE | 840 | 11.244 | 2.182 | −15.154 | 1.00 | 17.98 |
| ATOM | 5772 | CG1 | ILE | 840 | 12.249 | 0.111 | −16.168 | 1.00 | 18.15 |
| ATOM | 5773 | CD1 | ILE | 840 | 12.960 | −0.332 | −14.889 | 1.00 | 21.02 |
| ATOM | 5774 | C | ILE | 840 | 11.034 | 3.432 | −17.708 | 1.00 | 18.92 |
| ATOM | 5775 | O | ILE | 840 | 9.879 | 3.783 | −17.473 | 1.00 | 18.54 |
| ATOM | 5776 | N | ARG | 841 | 11.998 | 4.300 | −17.995 | 1.00 | 19.90 |
| ATOM | 5777 | CA | ARG | 841 | 11.698 | 5.722 | −18.067 | 1.00 | 19.12 |
| ATOM | 5778 | CB | ARG | 841 | 12.991 | 6.530 | −18.204 | 1.00 | 18.56 |
| ATOM | 5779 | CG | ARG | 841 | 13.814 | 6.523 | −16.916 | 1.00 | 20.62 |
| ATOM | 5780 | CD | ARG | 841 | 15.181 | 7.187 | −17.067 | 1.00 | 21.49 |
| ATOM | 5781 | NE | ARG | 841 | 15.852 | 7.308 | −15.774 | 1.00 | 21.81 |
| ATOM | 5782 | CZ | ARG | 841 | 16.347 | 6.286 | −15.082 | 1.00 | 22.30 |
| ATOM | 5783 | NH1 | ARG | 841 | 16.260 | 5.050 | −15.556 | 1.00 | 21.26 |
| ATOM | 5784 | NH2 | ARG | 841 | 16.914 | 6.501 | −13.902 | 1.00 | 22.06 |
| ATOM | 5785 | C | ARG | 841 | 10.743 | 5.987 | −19.232 | 1.00 | 18.94 |
| ATOM | 5786 | O | ARG | 841 | 9.842 | 6.822 | −19.132 | 1.00 | 17.46 |
| ATOM | 5787 | N | ALA | 842 | 10.918 | 5.262 | −20.331 | 1.00 | 19.89 |
| ATOM | 5788 | CA | ALA | 842 | 10.028 | 5.436 | −21.469 | 1.00 | 20.59 |
| ATOM | 5789 | CB | ALA | 842 | 10.558 | 4.679 | −22.677 | 1.00 | 22.80 |
| ATOM | 5790 | C | ALA | 842 | 8.638 | 4.921 | −21.091 | 1.00 | 20.77 |
| ATOM | 5791 | O | ALA | 842 | 7.624 | 5.422 | −21.583 | 1.00 | 22.42 |
| ATOM | 5792 | N | ALA | 843 | 8.591 | 3.922 | −20.214 | 1.00 | 19.81 |
| ATOM | 5793 | CA | ALA | 843 | 7.320 | 3.346 | −19.780 | 1.00 | 17.80 |
| ATOM | 5794 | CB | ALA | 843 | 7.566 | 2.046 | −19.019 | 1.00 | 19.09 |
| ATOM | 5795 | C | ALA | 843 | 6.539 | 4.321 | −18.910 | 1.00 | 17.75 |
| ATOM | 5796 | O | ALA | 843 | 5.310 | 4.411 | −19.002 | 1.00 | 15.71 |
| ATOM | 5797 | N | VAL | 844 | 7.259 | 5.045 | −18.059 | 1.00 | 17.72 |
| ATOM | 5798 | CA | VAL | 844 | 6.647 | 6.028 | −17.183 | 1.00 | 17.43 |
| ATOM | 5799 | CB | VAL | 844 | 7.683 | 6.616 | −16.185 | 1.00 | 15.55 |
| ATOM | 5800 | CG1 | VAL | 844 | 7.074 | 7.786 | −15.448 | 1.00 | 18.17 |
| ATOM | 5801 | CG2 | VAL | 844 | 8.113 | 5.545 | −15.169 | 1.00 | 15.06 |
| ATOM | 5802 | C | VAL | 844 | 6.063 | 7.159 | −18.029 | 1.00 | 19.69 |
| ATOM | 5803 | O | VAL | 844 | 4.942 | 7.606 | −17.790 | 1.00 | 19.85 |
| ATOM | 5804 | N | ARG | 845 | 6.817 | 7.618 | −19.024 | 1.00 | 20.79 |
| ATOM | 5805 | CA | ARG | 845 | 6.325 | 8.695 | −19.876 | 1.00 | 21.40 |
| ATOM | 5806 | CB | ARG | 845 | 7.394 | 9.118 | −20.886 | 1.00 | 23.26 |
| ATOM | 5807 | CG | ARG | 845 | 8.621 | 9.752 | −20.255 | 1.00 | 24.31 |
| ATOM | 5808 | CD | ARG | 845 | 9.502 | 10.422 | −21.298 | 1.00 | 25.50 |
| ATOM | 5809 | NE | ARG | 845 | 10.126 | 9.470 | −22.217 | 1.00 | 25.46 |
| ATOM | 5810 | CZ | ARG | 845 | 11.294 | 8.872 | −22.001 | 1.00 | 26.44 |
| ATOM | 5811 | NH1 | ARG | 845 | 11.976 | 9.121 | −20.890 | 1.00 | 27.41 |
| ATOM | 5812 | NH2 | ARG | 845 | 11.787 | 8.038 | −22.908 | 1.00 | 24.69 |
| ATOM | 5813 | C | ARG | 845 | 5.054 | 8.281 | −20.612 | 1.00 | 21.24 |
| ATOM | 5814 | O | ARG | 845 | 4.109 | 9.068 | −20.733 | 1.00 | 19.90 |
| ATOM | 5815 | N | GLN | 846 | 5.029 | 7.044 | −21.100 | 1.00 | 20.09 |
| ATOM | 5816 | CA | GLN | 846 | 3.862 | 6.549 | −21.816 | 1.00 | 20.80 |
| ATOM | 5817 | CB | GLN | 846 | 4.140 | 5.163 | −22.410 | 1.00 | 23.28 |
| ATOM | 5818 | CG | GLN | 846 | 2.932 | 4.529 | −23.092 | 1.00 | 30.74 |
| ATOM | 5819 | CD | GLN | 846 | 3.280 | 3.266 | −23.869 | 1.00 | 35.73 |
| ATOM | 5820 | OE1 | GLN | 846 | 3.947 | 3.324 | −24.904 | 1.00 | 38.70 |
| ATOM | 5821 | NE2 | GLN | 846 | 2.833 | 2.117 | −23.370 | 1.00 | 37.15 |
| ATOM | 5822 | C | GLN | 846 | 2.652 | 6.498 | −20.889 | 1.00 | 19.10 |
| ATOM | 5823 | O | GLN | 846 | 1.527 | 6.809 | −21.290 | 1.00 | 18.61 |
| ATOM | 5824 | N | TYR | 847 | 2.881 | 6.117 | −19.639 | 1.00 | 18.58 |
| ATOM | 5825 | CA | TYR | 847 | 1.790 | 6.051 | −18.676 | 1.00 | 18.17 |
| ATOM | 5826 | CB | TYR | 847 | 2.301 | 5.446 | −17.365 | 1.00 | 17.16 |
| ATOM | 5827 | CG | TYR | 847 | 1.364 | 5.597 | −16.189 | 1.00 | 14.88 |
| ATOM | 5828 | CD1 | TYR | 847 | 0.037 | 5.160 | −16.257 | 1.00 | 13.94 |
| ATOM | 5829 | CE1 | TYR | 847 | −0.810 | 5.277 | −15.156 | 1.00 | 14.43 |
| ATOM | 5830 | CD2 | TYR | 847 | 1.814 | 6.152 | −14.995 | 1.00 | 14.30 |
| ATOM | 5831 | CE2 | TYR | 847 | 0.980 | 6.272 | −13.897 | 1.00 | 13.19 |
| ATOM | 5832 | CZ | TYR | 847 | −0.321 | 5.839 | −13.975 | 1.00 | 14.15 |
| ATOM | 5833 | OH | TYR | 847 | −1.130 | 5.965 | −12.876 | 1.00 | 15.40 |
| ATOM | 5834 | C | TYR | 847 | 1.222 | 7.453 | −18.451 | 1.00 | 17.79 |
| ATOM | 5835 | O | TYR | 847 | 0.005 | 7.646 | −18.429 | 1.00 | 18.16 |
| ATOM | 5836 | N | MET | 848 | 2.112 | 8.430 | −18.299 | 1.00 | 19.31 |
| ATOM | 5837 | CA | MET | 848 | 1.705 | 9.813 | −18.089 | 1.00 | 19.23 |
| ATOM | 5838 | CB | MET | 848 | 2.946 | 10.700 | −17.931 | 1.00 | 21.20 |
| ATOM | 5839 | CG | MET | 848 | 3.724 | 10.462 | −16.639 | 1.00 | 21.47 |
| ATOM | 5840 | SD | MET | 848 | 5.424 | 11.088 | −16.716 | 1.00 | 22.85 |
| ATOM | 5841 | CE | MET | 848 | 5.109 | 12.850 | −16.646 | 1.00 | 23.08 |
| ATOM | 5842 | C | MET | 848 | 0.861 | 10.304 | −19.263 | 1.00 | 19.69 |
| ATOM | 5843 | O | MET | 848 | −0.208 | 10.882 | −19.075 | 1.00 | 19.25 |
| ATOM | 5844 | N | ALA | 849 | 1.339 | 10.051 | −20.476 | 1.00 | 20.45 |
| ATOM | 5845 | CA | ALA | 849 | 0.643 | 10.487 | −21.681 | 1.00 | 20.76 |
| ATOM | 5846 | CB | ALA | 849 | 1.537 | 10.280 | −22.894 | 1.00 | 20.70 |
| ATOM | 5847 | C | ALA | 849 | −0.701 | 9.797 | −21.892 | 1.00 | 21.08 |
| ATOM | 5848 | O | ALA | 849 | −1.699 | 10.456 | −22.169 | 1.00 | 22.23 |
| ATOM | 5849 | N | GLU | 850 | −0.740 | 8.475 | −21.761 | 1.00 | 19.51 |
| ATOM | 5850 | CA | GLU | 850 | −1.996 | 7.759 | −21.960 | 1.00 | 20.22 |
| ATOM | 5851 | CB | GLU | 850 | −1.751 | 6.250 | −21.977 | 1.00 | 20.03 |
| ATOM | 5852 | CG | GLU | 850 | −1.091 | 5.780 | −23.261 | 1.00 | 21.40 |
| ATOM | 5853 | CD | GLU | 850 | −0.997 | 4.282 | −23.355 | 1.00 | 22.09 |
| ATOM | 5854 | OE1 | GLU | 850 | −1.802 | 3.593 | −22.703 | 1.00 | 23.56 |
| ATOM | 5855 | OE2 | GLU | 850 | −0.124 | 3.792 | −24.096 | 1.00 | 27.29 |
| ATOM | 5856 | C | GLU | 850 | −3.075 | 8.108 | −20.942 | 1.00 | 20.17 |
| ATOM | 5857 | O | GLU | 850 | −4.268 | 7.947 | −21.211 | 1.00 | 20.44 |
| ATOM | 5858 | N | VAL | 851 | −2.666 | 8.574 | −19.765 | 1.00 | 20.20 |
| ATOM | 5859 | CA | VAL | 851 | −3.642 | 8.967 | −18.752 | 1.00 | 18.55 |
| ATOM | 5860 | CB | VAL | 851 | −2.995 | 9.092 | −17.352 | 1.00 | 19.02 |
| ATOM | 5861 | CG1 | VAL | 851 | −3.957 | 9.783 | −16.387 | 1.00 | 13.86 |
| ATOM | 5862 | CG2 | VAL | 851 | −2.663 | 7.700 | −16.820 | 1.00 | 17.16 |
| ATOM | 5863 | C | VAL | 851 | −4.266 | 10.305 | −19.154 | 1.00 | 18.95 |
| ATOM | 5864 | O | VAL | 851 | −5.489 | 10.453 | −19.167 | 1.00 | 17.91 |
| ATOM | 5865 | N | GLU | 852 | −3.424 | 11.270 | −19.506 | 1.00 | 20.35 |
| ATOM | 5866 | CA | GLU | 852 | −3.918 | 12.583 | −19.895 | 1.00 | 22.19 |
| ATOM | 5867 | CB | GLU | 852 | −2.748 | 13.567 | −20.023 | 1.00 | 24.34 |
| ATOM | 5868 | CG | GLU | 852 | −3.173 | 14.986 | −20.385 | 1.00 | 28.53 |
| ATOM | 5869 | CD | GLU | 852 | −2.073 | 16.009 | −20.170 | 1.00 | 30.90 |
| ATOM | 5870 | OE1 | GLU | 852 | −2.210 | 17.137 | −20.687 | 1.00 | 35.44 |
| ATOM | 5871 | OE2 | GLU | 852 | −1.079 | 15.696 | −19.480 | 1.00 | 31.33 |
| ATOM | 5872 | C | GLU | 852 | −4.745 | 12.560 | −21.186 | 1.00 | 22.60 |
| ATOM | 5873 | O | GLU | 852 | −5.634 | 13.393 | −21.376 | 1.00 | 21.58 |
| ATOM | 5874 | N | SER | 853 | −4.465 | 11.602 | −22.064 | 1.00 | 23.29 |
| ATOM | 5875 | CA | SER | 853 | −5.204 | 11.489 | −23.322 | 1.00 | 24.06 |
| ATOM | 5876 | CB | SER | 853 | −4.340 | 10.844 | −24.405 | 1.00 | 26.77 |
| ATOM | 5877 | OG | SER | 853 | −3.358 | 11.747 | −24.879 | 1.00 | 34.21 |
| ATOM | 5878 | C | SER | 853 | −6.473 | 10.671 | −23.162 | 1.00 | 23.46 |
| ATOM | 5879 | O | SER | 853 | −7.340 | 10.690 | −24.033 | 1.00 | 21.96 |
| ATOM | 5880 | N | GLY | 854 | −6.576 | 9.948 | −22.052 | 1.00 | 20.67 |
| ATOM | 5881 | CA | GLY | 854 | −7.752 | 9.131 | −21.822 | 1.00 | 21.47 |
| ATOM | 5882 | C | GLY | 854 | −7.620 | 7.744 | −22.424 | 1.00 | 21.43 |
| ATOM | 5883 | O | GLY | 854 | −8.552 | 6.947 | −22.343 | 1.00 | 22.20 |
| ATOM | 5884 | N | VAL | 855 | −6.472 | 7.459 | −23.032 | 1.00 | 21.89 |
| ATOM | 5885 | CA | VAL | 855 | −6.210 | 6.151 | −23.636 | 1.00 | 22.78 |
| ATOM | 5886 | CB | VAL | 855 | −4.871 | 6.155 | −24.394 | 1.00 | 23.64 |
| ATOM | 5887 | CG1 | VAL | 855 | −4.521 | 4.744 | −24.855 | 1.00 | 25.02 |
| ATOM | 5888 | CG2 | VAL | 855 | −4.967 | 7.087 | −25.588 | 1.00 | 27.01 |
| ATOM | 5889 | C | VAL | 855 | −6.160 | 5.071 | −22.562 | 1.00 | 22.02 |
| ATOM | 5890 | O | VAL | 855 | −6.572 | 3.928 | −22.787 | 1.00 | 21.37 |
| ATOM | 5891 | N | TYR | 856 | −5.632 | 5.448 | −21.400 | 1.00 | 20.96 |
| ATOM | 5892 | CA | TYR | 856 | −5.530 | 4.551 | −20.252 | 1.00 | 19.68 |
| ATOM | 5893 | CB | TYR | 856 | −4.073 | 4.276 | −19.887 | 1.00 | 17.64 |
| ATOM | 5894 | CG | TYR | 856 | −3.966 | 3.319 | −18.729 | 1.00 | 18.23 |
| ATOM | 5895 | CD1 | TYR | 856 | −4.153 | 1.951 | −18.923 | 1.00 | 17.91 |
| ATOM | 5896 | CE1 | TYR | 856 | −4.151 | 1.068 | −17.849 | 1.00 | 17.59 |
| ATOM | 5897 | CD2 | TYR | 856 | −3.766 | 3.782 | −17.424 | 1.00 | 19.26 |
| ATOM | 5898 | CE2 | TYR | 856 | −3.763 | 2.905 | −16.339 | 1.00 | 16.18 |
| ATOM | 5899 | CZ | TYR | 856 | −3.958 | 1.553 | −16.560 | 1.00 | 18.50 |
| ATOM | 5900 | OH | TYR | 856 | −3.975 | 0.676 | −15.494 | 1.00 | 19.57 |
| ATOM | 5901 | C | TYR | 856 | −6.208 | 5.195 | −19.048 | 1.00 | 19.48 |
| ATOM | 5902 | O | TYR | 856 | −5.951 | 6.360 | −18.735 | 1.00 | 18.59 |
| ATOM | 5903 | N | PRO | 857 | −7.057 | 4.435 | −18.339 | 1.00 | 21.09 |
| ATOM | 5904 | CD | PRO | 857 | −7.690 | 4.858 | −17.079 | 1.00 | 22.39 |
| ATOM | 5905 | CA | PRO | 857 | −7.373 | 3.034 | −18.631 | 1.00 | 22.76 |
| ATOM | 5906 | CB | PRO | 857 | −7.935 | 2.528 | −17.309 | 1.00 | 23.94 |
| ATOM | 5907 | CG | PRO | 857 | −8.658 | 3.724 | −16.794 | 1.00 | 24.29 |
| ATOM | 5908 | C | PRO | 857 | −8.355 | 2.860 | −19.784 | 1.00 | 23.68 |
| ATOM | 5909 | O | PRO | 857 | −9.263 | 3.670 | −19.970 | 1.00 | 22.22 |
| ATOM | 5910 | N | GLY | 858 | −8.166 | 1.797 | −20.558 | 1.00 | 24.27 |
| ATOM | 5911 | CA | GLY | 858 | −9.048 | 1.541 | −21.684 | 1.00 | 24.39 |
| ATOM | 5912 | C | GLY | 858 | −10.250 | 0.719 | −21.272 | 1.00 | 25.24 |
| ATOM | 5913 | O | GLY | 858 | −10.357 | 0.297 | −20.122 | 1.00 | 23.87 |
| ATOM | 5914 | N | GLU | 859 | −11.165 | 0.491 | −22.205 | 1.00 | 25.45 |
| ATOM | 5915 | CA | GLU | 859 | −12.344 | −0.302 | −21.891 | 1.00 | 28.69 |
| ATOM | 5916 | CB | GLU | 859 | −13.185 | −0.520 | −23.151 | 1.00 | 31.54 |
| ATOM | 5917 | CG | GLU | 859 | −14.435 | −1.339 | −22.908 | 1.00 | 34.32 |
| ATOM | 5918 | CD | GLU | 859 | −15.420 | −0.640 | −21.992 | 1.00 | 37.49 |
| ATOM | 5919 | OE1 | GLU | 859 | −16.335 | −1.318 | −21.482 | 1.00 | 40.17 |
| ATOM | 5920 | OE2 | GLU | 859 | −15.284 | 0.587 | −21.787 | 1.00 | 37.73 |
| ATOM | 5921 | C | GLU | 859 | −11.911 | −1.650 | −21.312 | 1.00 | 28.31 |
| ATOM | 5922 | O | GLU | 859 | −12.585 | −2.207 | −20.446 | 1.00 | 28.91 |
| ATOM | 5923 | N | GLU | 860 | −10.774 | −2.161 | −21.779 | 1.00 | 30.25 |
| ATOM | 5924 | CA | GLU | 860 | −10.255 | −3.448 | −21.313 | 1.00 | 31.44 |
| ATOM | 5925 | CB | GLU | 860 | −9.016 | −3.851 | −22.119 | 1.00 | 35.44 |
| ATOM | 5926 | CG | GLU | 860 | −9.077 | −3.503 | −23.593 | 1.00 | 41.38 |
| ATOM | 5927 | CD | GLU | 860 | −8.747 | −2.044 | −23.853 | 1.00 | 43.62 |
| ATOM | 5928 | OE1 | GLU | 860 | −7.599 | −1.637 | −23.565 | 1.00 | 44.99 |
| ATOM | 5929 | OE2 | GLU | 860 | −9.633 | −1.306 | −24.342 | 1.00 | 44.03 |
| ATOM | 5930 | C | GLU | 860 | −9.887 | −3.455 | −19.833 | 1.00 | 31.02 |
| ATOM | 5931 | O | GLU | 860 | −9.957 | −4.494 | −19.177 | 1.00 | 30.50 |
| ATOM | 5932 | N | HIS | 861 | −9.488 | −2.296 | −19.316 | 1.00 | 28.81 |
| ATOM | 5933 | CA | HIS | 861 | −9.087 | −2.169 | −17.915 | 1.00 | 27.99 |
| ATOM | 5934 | CB | HIS | 861 | −7.934 | −1.170 | −17.793 | 1.00 | 25.91 |
| ATOM | 5935 | CG | HIS | 861 | −6.871 | −1.344 | −18.828 | 1.00 | 24.41 |
| ATOM | 5936 | CD2 | HIS | 861 | −6.519 | −0.568 | −19.880 | 1.00 | 22.82 |
| ATOM | 5937 | ND1 | HIS | 861 | −6.026 | −2.432 | −18.855 | 1.00 | 25.29 |
| ATOM | 5938 | CE1 | HIS | 861 | −5.199 | −2.319 | −19.879 | 1.00 | 22.41 |
| ATOM | 5939 | NE2 | HIS | 861 | −5.478 | −1.197 | −20.518 | 1.00 | 24.27 |
| ATOM | 5940 | C | HIS | 861 | −10.235 | −1.677 | −17.044 | 1.00 | 27.60 |
| ATOM | 5941 | O | HIS | 861 | −10.042 | −1.385 | −15.866 | 1.00 | 27.53 |
| ATOM | 5942 | N | SER | 862 | −11.427 | −1.594 | −17.622 | 1.00 | 26.94 |
| ATOM | 5943 | CA | SER | 862 | −12.580 | −1.096 | −16.888 | 1.00 | 27.95 |
| ATOM | 5944 | CB | SER | 862 | −13.240 | 0.028 | −17.686 | 1.00 | 27.74 |
| ATOM | 5945 | OG | SER | 862 | −12.273 | 0.974 | −18.118 | 1.00 | 28.11 |
| ATOM | 5946 | C | SER | 862 | −13.620 | −2.160 | −16.548 | 1.00 | 29.39 |
| ATOM | 5947 | O | SER | 862 | −13.764 | −3.156 | −17.258 | 1.00 | 28.79 |
| ATOM | 5948 | N | PHE | 863 | −14.336 | −1.936 | −15.447 | 1.00 | 29.95 |
| ATOM | 5949 | CA | PHE | 863 | −15.385 | −2.842 | −14.998 | 1.00 | 32.29 |
| ATOM | 5950 | CB | PHE | 863 | −15.257 | −3.141 | −13.498 | 1.00 | 30.67 |
| ATOM | 5951 | CG | PHE | 863 | −13.991 | −3.858 | −13.120 | 1.00 | 31.07 |
| ATOM | 5952 | CD1 | PHE | 863 | −12.912 | −3.160 | −12.587 | 1.00 | 31.09 |
| ATOM | 5953 | CD2 | PHE | 863 | −13.875 | −5.234 | −13.298 | 1.00 | 31.78 |
| ATOM | 5954 | CE1 | PHE | 863 | −11.737 | −3.820 | −12.231 | 1.00 | 30.30 |
| ATOM | 5955 | CE2 | PHE | 863 | −12.703 | −5.905 | −12.947 | 1.00 | 32.24 |
| ATOM | 5956 | CZ | PHE | 863 | −11.633 | −5.195 | −12.413 | 1.00 | 32.64 |
| ATOM | 5957 | C | PHE | 863 | −16.741 | −2.197 | −15.261 | 1.00 | 34.00 |
| ATOM | 5958 | O | PHE | 863 | −16.819 | −1.031 | −15.651 | 1.00 | 34.45 |
| ATOM | 5959 | N | HIS | 864 | −17.807 | −2.959 | −15.042 | 1.00 | 35.05 |
| ATOM | 5960 | CA | HIS | 864 | −19.163 | −2.465 | −15.252 | 1.00 | 36.87 |
| ATOM | 5961 | CB | HIS | 864 | −19.600 | −2.700 | −16.700 | 1.00 | 37.63 |
| ATOM | 5962 | CG | HIS | 864 | −18.935 | −1.789 | −17.682 | 1.00 | 37.41 |
| ATOM | 5963 | CD2 | HIS | 864 | −18.064 | −2.042 | −18.688 | 1.00 | 38.33 |
| ATOM | 5964 | ND1 | HIS | 864 | −19.130 | −0.425 | −17.678 | 1.00 | 38.08 |
| ATOM | 5965 | CE1 | HIS | 864 | −18.408 | 0.123 | −18.639 | 1.00 | 37.64 |
| ATOM | 5966 | NE2 | HIS | 864 | −17.752 | −0.835 | −19.266 | 1.00 | 37.77 |
| ATOM | 5967 | C | HIS | 864 | −20.132 | −3.147 | −14.305 | 1.00 | 37.62 |
| ATOM | 5968 | O | HIS | 864 | −21.218 | −2.579 | −14.068 | 1.00 | 38.24 |
| ATOM | 5969 | OXT | HIS | 864 | −19.794 | −4.250 | −13.826 | 1.00 | 39.23 |
| ATOM | 5970 | C1 | KPL | 865 | −3.357 | 0.634 | −5.095 | 1.00 | 39.96 |
| ATOM | 5971 | C2 | KPL | 865 | −3.550 | 1.896 | −4.226 | 1.00 | 38.72 |
| ATOM | 5972 | C3 | KPL | 865 | −2.617 | 2.997 | −4.740 | 1.00 | 39.62 |
| ATOM | 5973 | C4 | KPL | 865 | −5.003 | 2.393 | −4.350 | 1.00 | 40.42 |
| ATOM | 5974 | O1 | KPL | 865 | −5.910 | 1.387 | −3.884 | 1.00 | 45.23 |
| ATOM | 5975 | C5 | KPL | 865 | −3.211 | 1.589 | −2.749 | 1.00 | 35.98 |
| ATOM | 5976 | O2 | KPL | 865 | −4.048 | 1.753 | −1.886 | 1.00 | 36.91 |
| ATOM | 5977 | C6 | KPL | 865 | −1.855 | 1.081 | −2.331 | 1.00 | 32.68 |
| ATOM | 5978 | O3 | KPL | 865 | −0.975 | 0.900 | −3.150 | 1.00 | 30.34 |
| ATOM | 5979 | O4 | KPL | 865 | −1.620 | 0.826 | −1.028 | 1.00 | 27.08 |
| ATOM | 5980 | CB | MET | 901 | −12.712 | −23.902 | −0.148 | 1.00 | 60.92 |
| ATOM | 5981 | CG | MET | 901 | −12.590 | −25.152 | −1.024 | 1.00 | 62.73 |
| ATOM | 5982 | SD | MET | 901 | −10.891 | −25.631 | −1.435 | 1.00 | 65.23 |
| ATOM | 5983 | CE | MET | 901 | −10.776 | −25.064 | −3.145 | 1.00 | 64.55 |
| ATOM | 5984 | C | MET | 901 | −10.847 | −22.440 | −0.981 | 1.00 | 58.43 |
| ATOM | 5985 | O | MET | 901 | −10.083 | −23.083 | −0.258 | 1.00 | 58.56 |
| ATOM | 5986 | N | MET | 901 | −13.083 | −22.466 | −2.137 | 1.00 | 59.24 |
| ATOM | 5987 | CA | MET | 901 | −12.364 | −22.573 | −0.834 | 1.00 | 59.60 |
| ATOM | 5988 | N | LYS | 902 | −10.415 | −21.594 | −1.912 | 1.00 | 56.21 |
| ATOM | 5989 | CA | LYS | 902 | −8.991 | −21.379 | −2.147 | 1.00 | 52.69 |
| ATOM | 5990 | CB | LYS | 902 | −8.505 | −22.296 | −3.281 | 1.00 | 54.41 |
| ATOM | 5991 | CG | LYS | 902 | −6.987 | −22.462 | −3.364 | 1.00 | 55.97 |
| ATOM | 5992 | CD | LYS | 902 | −6.322 | −21.347 | −4.158 | 1.00 | 57.24 |
| ATOM | 5993 | CE | LYS | 902 | −6.660 | −21.451 | −5.641 | 1.00 | 58.67 |
| ATOM | 5994 | NZ | LYS | 902 | −5.962 | −20.416 | −6.455 | 1.00 | 59.28 |
| ATOM | 5995 | C | LYS | 902 | −8.721 | −19.915 | −2.491 | 1.00 | 49.10 |
| ATOM | 5996 | O | LYS | 902 | −8.688 | −19.538 | −3.664 | 1.00 | 49.44 |
| ATOM | 5997 | N | PRO | 903 | −8.542 | −19.065 | −1.465 | 1.00 | 44.68 |
| ATOM | 5998 | CD | PRO | 903 | −8.096 | −17.671 | −1.635 | 1.00 | 43.26 |
| ATOM | 5999 | CA | PRO | 903 | −8.590 | −19.422 | −0.042 | 1.00 | 40.59 |
| ATOM | 6000 | CB | PRO | 903 | −7.827 | −18.281 | 0.615 | 1.00 | 41.44 |
| ATOM | 6001 | CG | PRO | 903 | −8.221 | −17.114 | −0.230 | 1.00 | 42.64 |
| ATOM | 6002 | C | PRO | 903 | −10.016 | −19.540 | 0.488 | 1.00 | 37.14 |
| ATOM | 6003 | O | PRO | 903 | −10.957 | −19.042 | −0.125 | 1.00 | 35.87 |
| ATOM | 6004 | N | THR | 904 | −10.175 | −20.205 | 1.628 | 1.00 | 32.92 |
| ATOM | 6005 | CA | THR | 904 | −11.494 | −20.369 | 2.227 | 1.00 | 29.48 |
| ATOM | 6006 | CB | THR | 904 | −11.504 | −21.543 | 3.238 | 1.00 | 29.39 |
| ATOM | 6007 | OG1 | THR | 904 | −11.161 | −22.764 | 2.565 | 1.00 | 26.97 |
| ATOM | 6008 | CG2 | THR | 904 | −12.873 | −21.690 | 3.869 | 1.00 | 26.63 |
| ATOM | 6009 | C | THR | 904 | −11.857 | −19.073 | 2.950 | 1.00 | 28.40 |
| ATOM | 6010 | O | THR | 904 | −11.066 | −18.560 | 3.739 | 1.00 | 27.37 |
| ATOM | 6011 | N | THR | 905 | −13.047 | −18.543 | 2.675 | 1.00 | 28.09 |
| ATOM | 6012 | CA | THR | 905 | −13.487 | −17.300 | 3.308 | 1.00 | 28.38 |
| ATOM | 6013 | CB | THR | 905 | −13.470 | −16.122 | 2.310 | 1.00 | 29.55 |
| ATOM | 6014 | OG1 | THR | 905 | −14.342 | −16.416 | 1.211 | 1.00 | 31.91 |
| ATOM | 6015 | CG2 | THR | 905 | −12.066 | −15.873 | 1.796 | 1.00 | 31.52 |
| ATOM | 6016 | C | THR | 905 | −14.894 | −17.398 | 3.888 | 1.00 | 27.90 |
| ATOM | 6017 | O | THR | 905 | −15.603 | −18.380 | 3.670 | 1.00 | 26.37 |
| ATOM | 6018 | N | ILE | 906 | −15.288 | −16.370 | 4.633 | 1.00 | 28.35 |
| ATOM | 6019 | CA | ILE | 906 | −16.611 | −16.331 | 5.244 | 1.00 | 29.75 |
| ATOM | 6020 | CB | ILE | 906 | −16.854 | −14.979 | 5.961 | 1.00 | 30.43 |
| ATOM | 6021 | CG2 | ILE | 906 | −18.113 | −15.061 | 6.819 | 1.00 | 29.82 |
| ATOM | 6022 | CG1 | ILE | 906 | −15.659 | −14.641 | 6.856 | 1.00 | 32.57 |
| ATOM | 6023 | CD1 | ILE | 906 | −15.638 | −13.192 | 7.341 | 1.00 | 34.53 |
| ATOM | 6024 | C | ILE | 906 | −17.661 | −16.505 | 4.151 | 1.00 | 30.16 |
| ATOM | 6025 | O | ILE | 906 | −18.729 | −17.073 | 4.384 | 1.00 | 29.90 |
| ATOM | 6026 | N | SER | 907 | −17.340 | −16.021 | 2.955 | 1.00 | 31.30 |
| ATOM | 6027 | CA | SER | 907 | −18.244 | −16.107 | 1.810 | 1.00 | 32.70 |
| ATOM | 6028 | CB | SER | 907 | −17.545 | −15.593 | 0.547 | 1.00 | 33.26 |
| ATOM | 6029 | OG | SER | 907 | −16.935 | −14.332 | 0.770 | 1.00 | 34.50 |
| ATOM | 6030 | C | SER | 907 | −18.700 | −17.544 | 1.581 | 1.00 | 32.03 |
| ATOM | 6031 | O | SER | 907 | −19.860 | −17.793 | 1.255 | 1.00 | 31.78 |
| ATOM | 6032 | N | LEU | 908 | −17.777 | −18.485 | 1.757 | 1.00 | 32.10 |
| ATOM | 6033 | CA | LEU | 908 | −18.071 | −19.900 | 1.563 | 1.00 | 31.83 |
| ATOM | 6034 | CB | LEU | 908 | −16.783 | −20.723 | 1.643 | 1.00 | 33.59 |
| ATOM | 6035 | CG | LEU | 908 | −16.563 | −21.785 | 0.566 | 1.00 | 35.17 |
| ATOM | 6036 | CD1 | LEU | 908 | −15.600 | −22.822 | 1.103 | 1.00 | 34.73 |
| ATOM | 6037 | CD2 | LEU | 908 | −17.877 | −22.441 | 0.173 | 1.00 | 36.87 |
| ATOM | 6038 | C | LEU | 908 | −19.073 | −20.437 | 2.586 | 1.00 | 30.86 |
| ATOM | 6039 | O | LEU | 908 | −20.056 | −21.087 | 2.223 | 1.00 | 31.03 |
| ATOM | 6040 | N | LEU | 909 | −18.818 | −20.176 | 3.865 | 1.00 | 29.63 |
| ATOM | 6041 | CA | LEU | 909 | −19.705 | −20.643 | 4.926 | 1.00 | 28.90 |
| ATOM | 6042 | CB | LEU | 909 | −19.179 | −20.219 | 6.297 | 1.00 | 26.59 |
| ATOM | 6043 | CG | LEU | 909 | −17.783 | −20.723 | 6.669 | 1.00 | 26.10 |
| ATOM | 6044 | CD1 | LEU | 909 | −17.476 | −20.315 | 8.102 | 1.00 | 25.81 |
| ATOM | 6045 | CD2 | LEU | 909 | −17.721 | −22.229 | 6.519 | 1.00 | 27.17 |
| ATOM | 6046 | C | LEU | 909 | −21.109 | −20.093 | 4.750 | 1.00 | 29.37 |
| ATOM | 6047 | O | LEU | 909 | −22.092 | −20.765 | 5.056 | 1.00 | 28.78 |
| ATOM | 6048 | N | GLN | 910 | −21.198 | −18.861 | 4.266 | 1.00 | 30.45 |
| ATOM | 6049 | CA | GLN | 910 | −22.494 | −18.234 | 4.050 | 1.00 | 33.43 |
| ATOM | 6050 | CB | GLN | 910 | −22.306 | −16.767 | 3.659 | 1.00 | 33.71 |
| ATOM | 6051 | CG | GLN | 910 | −23.589 | −15.949 | 3.626 | 1.00 | 37.45 |
| ATOM | 6052 | CD | GLN | 910 | −24.345 | −15.976 | 4.944 | 1.00 | 37.83 |
| ATOM | 6053 | OE1 | GLN | 910 | −25.058 | −16.935 | 5.250 | 1.00 | 38.97 |
| ATOM | 6054 | NE2 | GLN | 910 | −24.182 | −14.924 | 5.738 | 1.00 | 36.67 |
| ATOM | 6055 | C | GLN | 910 | −23.205 | −19.006 | 2.945 | 1.00 | 33.53 |
| ATOM | 6056 | O | GLN | 910 | −24.405 | −19.258 | 3.028 | 1.00 | 33.64 |
| ATOM | 6057 | N | LYS | 911 | −22.451 | −19.389 | 1.919 | 1.00 | 35.95 |
| ATOM | 6058 | CA | LYS | 911 | −23.004 | −20.154 | 0.811 | 1.00 | 36.69 |
| ATOM | 6059 | CB | LYS | 911 | −21.947 | −20.387 | −0.269 | 1.00 | 38.54 |
| ATOM | 6060 | CG | LYS | 911 | −22.480 | −21.146 | −1.478 | 1.00 | 41.44 |
| ATOM | 6061 | CD | LYS | 911 | −21.484 | −22.170 | −2.010 | 1.00 | 42.22 |
| ATOM | 6062 | CE | LYS | 911 | −20.210 | −21.525 | −2.522 | 1.00 | 43.30 |
| ATOM | 6063 | NZ | LYS | 911 | −19.319 | −22.540 | −3.158 | 1.00 | 44.28 |
| ATOM | 6064 | C | LYS | 911 | −23.488 | −21.504 | 1.326 | 1.00 | 36.17 |
| ATOM | 6065 | O | LYS | 911 | −24.545 | −21.989 | 0.927 | 1.00 | 36.29 |
| ATOM | 6066 | N | TYR | 912 | −22.702 | −22.108 | 2.214 | 1.00 | 35.44 |
| ATOM | 6067 | CA | TYR | 912 | −23.041 | −23.407 | 2.789 | 1.00 | 33.78 |
| ATOM | 6068 | CB | TYR | 912 | −21.945 | −23.862 | 3.758 | 1.00 | 35.49 |
| ATOM | 6069 | CG | TYR | 912 | −20.707 | −24.422 | 3.091 | 1.00 | 37.41 |
| ATOM | 6070 | CD1 | TYR | 912 | −19.533 | −24.628 | 3.820 | 1.00 | 38.67 |
| ATOM | 6071 | CE1 | TYR | 912 | −18.395 | −25.159 | 3.217 | 1.00 | 39.92 |
| ATOM | 6072 | CD2 | TYR | 912 | −20.710 | −24.766 | 1.737 | 1.00 | 38.60 |
| ATOM | 6073 | CE2 | TYR | 912 | −19.580 | −25.299 | 1.127 | 1.00 | 40.16 |
| ATOM | 6074 | CZ | TYR | 912 | −18.428 | −25.493 | 1.871 | 1.00 | 40.30 |
| ATOM | 6075 | OH | TYR | 912 | −17.310 | −26.021 | 1.270 | 1.00 | 42.12 |
| ATOM | 6076 | C | TYR | 912 | −24.375 | −23.390 | 3.516 | 1.00 | 33.39 |
| ATOM | 6077 | O | TYR | 912 | −25.128 | −24.359 | 3.460 | 1.00 | 31.55 |
| ATOM | 6078 | N | LYS | 913 | −24.663 | −22.297 | 4.211 | 1.00 | 32.88 |
| ATOM | 6079 | CA | LYS | 913 | −25.920 | −22.202 | 4.936 | 1.00 | 35.58 |
| ATOM | 6080 | CB | LYS | 913 | −25.914 | −20.999 | 5.878 | 1.00 | 33.03 |
| ATOM | 6081 | CG | LYS | 913 | −27.182 | −20.898 | 6.697 | 1.00 | 32.07 |
| ATOM | 6082 | CD | LYS | 913 | −27.072 | −19.884 | 7.815 | 1.00 | 29.62 |
| ATOM | 6083 | CE | LYS | 913 | −28.360 | −19.861 | 8.616 | 1.00 | 28.34 |
| ATOM | 6084 | NZ | LYS | 913 | −28.225 | −19.120 | 9.891 | 1.00 | 28.41 |
| ATOM | 6085 | C | LYS | 913 | −27.092 | −22.086 | 3.968 | 1.00 | 37.48 |
| ATOM | 6086 | O | LYS | 913 | −28.156 | −22.656 | 4.203 | 1.00 | 37.96 |
| ATOM | 6087 | N | GLN | 914 | −26.891 | −21.346 | 2.883 | 1.00 | 40.52 |
| ATOM | 6088 | CA | GLN | 914 | −27.931 | −21.161 | 1.880 | 1.00 | 43.74 |
| ATOM | 6089 | CB | GLN | 914 | −27.498 | −20.122 | 0.845 | 1.00 | 45.66 |
| ATOM | 6090 | CG | GLN | 914 | −27.215 | −18.745 | 1.427 | 1.00 | 48.82 |
| ATOM | 6091 | CD | GLN | 914 | −26.988 | −17.695 | 0.355 | 1.00 | 51.41 |
| ATOM | 6092 | OE1 | GLN | 914 | −27.882 | −17.405 | −0.442 | 1.00 | 53.67 |
| ATOM | 6093 | NE2 | GLN | 914 | −25.788 | −17.122 | 0.327 | 1.00 | 52.30 |
| ATOM | 6094 | C | GLN | 914 | −28.227 | −22.481 | 1.183 | 1.00 | 44.63 |
| ATOM | 6095 | O | GLN | 914 | −29.357 | −22.735 | 0.768 | 1.00 | 44.57 |
| ATOM | 6096 | N | GLU | 915 | −27.198 | −23.314 | 1.058 | 1.00 | 45.74 |
| ATOM | 6097 | CA | GLU | 915 | −27.327 | −24.617 | 0.418 | 1.00 | 46.23 |
| ATOM | 6098 | CB | GLU | 915 | −26.026 | −24.988 | −0.298 | 1.00 | 47.95 |
| ATOM | 6099 | CG | GLU | 915 | −25.516 | −23.913 | −1.243 | 1.00 | 51.08 |
| ATOM | 6100 | CD | GLU | 915 | −24.213 | −24.299 | −1.917 | 1.00 | 52.26 |
| ATOM | 6101 | OE1 | GLU | 915 | −23.270 | −24.711 | −1.205 | 1.00 | 53.21 |
| ATOM | 6102 | OE2 | GLU | 915 | −24.127 | −24.181 | −3.157 | 1.00 | 53.18 |
| ATOM | 6103 | C | GLU | 915 | −27.654 | −25.675 | 1.463 | 1.00 | 45.27 |
| ATOM | 6104 | O | GLU | 915 | −27.700 | −26.867 | 1.160 | 1.00 | 45.43 |
| ATOM | 6105 | N | LYS | 916 | −27.874 | −25.233 | 2.697 | 1.00 | 44.09 |
| ATOM | 6106 | CA | LYS | 916 | −28.206 | −26.138 | 3.792 | 1.00 | 43.28 |
| ATOM | 6107 | CB | LYS | 916 | −29.587 | −26.754 | 3.558 | 1.00 | 45.07 |
| ATOM | 6108 | CG | LYS | 916 | −30.712 | −25.741 | 3.545 | 1.00 | 46.88 |
| ATOM | 6109 | CD | LYS | 916 | −30.921 | −25.137 | 4.921 | 1.00 | 48.53 |
| ATOM | 6110 | CE | LYS | 916 | −31.908 | −23.981 | 4.871 | 1.00 | 50.30 |
| ATOM | 6111 | NZ | LYS | 916 | −33.206 | −24.382 | 4.267 | 1.00 | 51.08 |
| ATOM | 6112 | C | LYS | 916 | −27.177 | −27.249 | 3.976 | 1.00 | 42.34 |
| ATOM | 6113 | O | LYS | 916 | −27.519 | −28.359 | 4.382 | 1.00 | 42.68 |
| ATOM | 6114 | N | LYS | 917 | −25.918 | −26.949 | 3.673 | 1.00 | 40.67 |
| ATOM | 6115 | CA | LYS | 917 | −24.849 | −27.928 | 3.823 | 1.00 | 38.46 |
| ATOM | 6116 | CB | LYS | 917 | −23.865 | −27.841 | 2.654 | 1.00 | 39.13 |
| ATOM | 6117 | CG | LYS | 917 | −22.696 | −28.814 | 2.781 | 1.00 | 41.58 |
| ATOM | 6118 | CD | LYS | 917 | −21.540 | −28.465 | 1.851 | 1.00 | 44.05 |
| ATOM | 6119 | CE | LYS | 917 | −21.903 | −28.642 | 0.387 | 1.00 | 46.51 |
| ATOM | 6120 | NZ | LYS | 917 | −20.749 | −28.300 | −0.494 | 1.00 | 48.27 |
| ATOM | 6121 | C | LYS | 917 | −24.095 | −27.675 | 5.123 | 1.00 | 36.66 |
| ATOM | 6122 | O | LYS | 917 | −23.297 | −26.741 | 5.211 | 1.00 | 35.67 |
| ATOM | 6123 | N | ARG | 918 | −24.355 | −28.504 | 6.129 | 1.00 | 33.82 |
| ATOM | 6124 | CA | ARG | 918 | −23.689 | −28.373 | 7.420 | 1.00 | 31.81 |
| ATOM | 6125 | CB | ARG | 918 | −24.297 | −29.362 | 8.426 | 1.00 | 30.96 |
| ATOM | 6126 | CG | ARG | 918 | −25.668 | −28.926 | 8.931 | 1.00 | 32.18 |
| ATOM | 6127 | CD | ARG | 918 | −26.346 | −29.957 | 9.826 | 1.00 | 33.25 |
| ATOM | 6128 | NE | ARG | 918 | −26.812 | −31.128 | 9.084 | 1.00 | 33.83 |
| ATOM | 6129 | CZ | ARG | 918 | −27.706 | −32.000 | 9.544 | 1.00 | 33.54 |
| ATOM | 6130 | NH1 | ARG | 918 | −28.240 | −31.840 | 10.747 | 1.00 | 33.73 |
| ATOM | 6131 | NH2 | ARG | 918 | −28.071 | −33.036 | 8.801 | 1.00 | 35.50 |
| ATOM | 6132 | C | ARG | 918 | −22.192 | −28.624 | 7.242 | 1.00 | 29.61 |
| ATOM | 6133 | O | ARG | 918 | −21.792 | −29.565 | 6.557 | 1.00 | 29.96 |
| ATOM | 6134 | N | PHE | 919 | −21.368 | −27.778 | 7.857 | 1.00 | 26.58 |
| ATOM | 6135 | CA | PHE | 919 | −19.911 | −27.890 | 7.738 | 1.00 | 24.02 |
| ATOM | 6136 | CB | PHE | 919 | −19.368 | −26.625 | 7.062 | 1.00 | 24.17 |
| ATOM | 6137 | CG | PHE | 919 | −19.743 | −25.350 | 7.771 | 1.00 | 23.99 |
| ATOM | 6138 | CD1 | PHE | 919 | −18.925 | −24.820 | 8.769 | 1.00 | 22.22 |
| ATOM | 6139 | CD2 | PHE | 919 | −20.932 | −24.691 | 7.459 | 1.00 | 23.20 |
| ATOM | 6140 | CE1 | PHE | 919 | −19.286 | −23.657 | 9.440 | 1.00 | 22.28 |
| ATOM | 6141 | CE2 | PHE | 919 | −21.299 | −23.528 | 8.127 | 1.00 | 23.01 |
| ATOM | 6142 | CZ | PHE | 919 | −20.475 | −23.009 | 9.120 | 1.00 | 23.76 |
| ATOM | 6143 | C | PHE | 919 | −19.206 | −28.097 | 9.076 | 1.00 | 23.65 |
| ATOM | 6144 | O | PHE | 919 | −19.679 | −27.636 | 10.119 | 1.00 | 22.72 |
| ATOM | 6145 | N | ALA | 920 | −18.071 | −28.789 | 9.049 | 1.00 | 21.57 |
| ATOM | 6146 | CA | ALA | 920 | −17.313 | −29.028 | 10.276 | 1.00 | 19.22 |
| ATOM | 6147 | CB | ALA | 920 | −16.709 | −30.436 | 10.260 | 1.00 | 18.52 |
| ATOM | 6148 | C | ALA | 920 | −16.213 | −27.988 | 10.445 | 1.00 | 17.19 |
| ATOM | 6149 | O | ALA | 920 | −15.645 | −27.506 | 9.463 | 1.00 | 17.07 |
| ATOM | 6150 | N | THR | 921 | −15.932 | −27.644 | 11.701 | 1.00 | 17.33 |
| ATOM | 6151 | CA | THR | 921 | −14.904 | −26.662 | 12.071 | 1.00 | 17.47 |
| ATOM | 6152 | CB | THR | 921 | −15.550 | −25.358 | 12.581 | 1.00 | 20.30 |
| ATOM | 6153 | OG1 | THR | 921 | −16.349 | −24.781 | 11.533 | 1.00 | 24.13 |
| ATOM | 6154 | CG2 | THR | 921 | −14.492 | −24.372 | 12.999 | 1.00 | 27.64 |
| ATOM | 6155 | C | THR | 921 | −14.091 | −27.293 | 13.205 | 1.00 | 14.38 |
| ATOM | 6156 | O | THR | 921 | −14.586 | −28.182 | 13.892 | 1.00 | 13.90 |
| ATOM | 6157 | N | ILE | 922 | −12.861 | −26.843 | 13.424 | 1.00 | 13.81 |
| ATOM | 6158 | CA | ILE | 922 | −12.054 | −27.454 | 14.481 | 1.00 | 13.12 |
| ATOM | 6159 | CB | ILE | 922 | −11.367 | −28.728 | 13.943 | 1.00 | 15.52 |
| ATOM | 6160 | CG2 | ILE | 922 | −10.274 | −28.341 | 12.925 | 1.00 | 15.80 |
| ATOM | 6161 | CG1 | ILE | 922 | −10.771 | −29.531 | 15.102 | 1.00 | 15.54 |
| ATOM | 6162 | CD1 | ILE | 922 | −10.400 | −30.953 | 14.734 | 1.00 | 25.56 |
| ATOM | 6163 | C | ILE | 922 | −10.988 | −26.514 | 15.040 | 1.00 | 13.33 |
| ATOM | 6164 | O | ILE | 922 | −10.591 | −25.555 | 14.375 | 1.00 | 13.71 |
| ATOM | 6165 | N | THR | 923 | −10.533 | −26.778 | 16.262 | 1.00 | 13.37 |
| ATOM | 6166 | CA | THR | 923 | −9.475 | −25.955 | 16.844 | 1.00 | 11.94 |
| ATOM | 6167 | CB | THR | 923 | −9.471 | −25.979 | 18.403 | 1.00 | 11.46 |
| ATOM | 6168 | OG1 | THR | 923 | −9.137 | −27.294 | 18.866 | 1.00 | 14.02 |
| ATOM | 6169 | CG2 | THR | 923 | −10.842 | −25.564 | 18.962 | 1.00 | 12.48 |
| ATOM | 6170 | C | THR | 923 | −8.130 | −26.494 | 16.358 | 1.00 | 11.04 |
| ATOM | 6171 | O | THR | 923 | −8.010 | −27.662 | 15.998 | 1.00 | 11.93 |
| ATOM | 6172 | N | ALA | 924 | −7.133 | −25.616 | 16.324 | 1.00 | 10.01 |
| ATOM | 6173 | CA | ALA | 924 | −5.783 | −25.965 | 15.908 | 1.00 | 9.37 |
| ATOM | 6174 | CB | ALA | 924 | −5.634 | −25.881 | 14.390 | 1.00 | 10.66 |
| ATOM | 6175 | C | ALA | 924 | −4.876 | −24.948 | 16.574 | 1.00 | 9.13 |
| ATOM | 6176 | O | ALA | 924 | −5.262 | −23.791 | 16.738 | 1.00 | 9.96 |
| ATOM | 6177 | N | TYR | 925 | −3.674 | −25.367 | 16.955 | 1.00 | 8.67 |
| ATOM | 6178 | CA | TYR | 925 | −2.757 | −24.463 | 17.638 | 1.00 | 10.02 |
| ATOM | 6179 | CB | TYR | 925 | −2.809 | −24.709 | 19.143 | 1.00 | 9.74 |
| ATOM | 6180 | CG | TYR | 925 | −4.188 | −25.007 | 19.687 | 1.00 | 10.81 |
| ATOM | 6181 | CD1 | TYR | 925 | −4.619 | −26.328 | 19.842 | 1.00 | 11.73 |
| ATOM | 6182 | CE1 | TYR | 925 | −5.871 | −26.625 | 20.373 | 1.00 | 12.92 |
| ATOM | 6183 | CD2 | TYR | 925 | −5.051 | −23.982 | 20.069 | 1.00 | 11.21 |
| ATOM | 6184 | CE2 | TYR | 925 | −6.315 | −24.269 | 20.603 | 1.00 | 10.91 |
| ATOM | 6185 | CZ | TYR | 925 | −6.711 | −25.592 | 20.752 | 1.00 | 12.01 |
| ATOM | 6186 | OH | TYR | 925 | −7.945 | −25.890 | 21.293 | 1.00 | 13.23 |
| ATOM | 6187 | C | TYR | 925 | −1.326 | −24.619 | 17.174 | 1.00 | 9.34 |
| ATOM | 6188 | O | TYR | 925 | −0.420 | −24.017 | 17.744 | 1.00 | 10.90 |
| ATOM | 6189 | N | ASP | 926 | −1.106 | −25.447 | 16.161 | 1.00 | 8.96 |
| ATOM | 6190 | CA | ASP | 926 | 0.250 | −25.626 | 15.672 | 1.00 | 10.92 |
| ATOM | 6191 | CB | ASP | 926 | 0.971 | −26.694 | 16.509 | 1.00 | 11.44 |
| ATOM | 6192 | CG | ASP | 926 | 0.378 | −28.093 | 16.328 | 1.00 | 11.32 |
| ATOM | 6193 | OD1 | ASP | 926 | 0.546 | −28.668 | 15.247 | 1.00 | 11.50 |
| ATOM | 6194 | OD2 | ASP | 926 | −0.257 | −28.601 | 17.274 | 1.00 | 15.55 |
| ATOM | 6195 | C | ASP | 926 | 0.283 | −25.982 | 14.186 | 1.00 | 11.52 |
| ATOM | 6196 | O | ASP | 926 | −0.748 | −26.292 | 13.581 | 1.00 | 13.19 |
| ATOM | 6197 | N | TYR | 927 | 1.477 | −25.929 | 13.610 | 1.00 | 9.04 |
| ATOM | 6198 | CA | TYR | 927 | 1.693 | −26.206 | 12.190 | 1.00 | 9.91 |
| ATOM | 6199 | CB | TYR | 927 | 3.183 | −26.037 | 11.848 | 1.00 | 12.47 |
| ATOM | 6200 | CG | TYR | 927 | 3.599 | −26.566 | 10.486 | 1.00 | 13.30 |
| ATOM | 6201 | CD1 | TYR | 927 | 3.408 | −25.805 | 9.332 | 1.00 | 15.77 |
| ATOM | 6202 | CE1 | TYR | 927 | 3.776 | −26.286 | 8.072 | 1.00 | 17.93 |
| ATOM | 6203 | CD2 | TYR | 927 | 4.175 | −27.831 | 10.351 | 1.00 | 14.44 |
| ATOM | 6204 | CE2 | TYR | 927 | 4.548 | −28.327 | 9.092 | 1.00 | 15.72 |
| ATOM | 6205 | CZ | TYR | 927 | 4.345 | −27.552 | 7.961 | 1.00 | 18.75 |
| ATOM | 6206 | OH | TYR | 927 | 4.694 | −28.034 | 6.708 | 1.00 | 20.14 |
| ATOM | 6207 | C | TYR | 927 | 1.256 | −27.588 | 11.745 | 1.00 | 12.26 |
| ATOM | 6208 | O | TYR | 927 | 0.494 | −27.732 | 10.791 | 1.00 | 12.34 |
| ATOM | 6209 | N | SER | 928 | 1.753 | −28.602 | 12.446 | 1.00 | 11.45 |
| ATOM | 6210 | CA | SER | 928 | 1.468 | −29.987 | 12.097 | 1.00 | 11.62 |
| ATOM | 6211 | CB | SER | 928 | 2.167 | −30.928 | 13.077 | 1.00 | 12.06 |
| ATOM | 6212 | OG | SER | 928 | 3.577 | −30.869 | 12.883 | 1.00 | 14.47 |
| ATOM | 6213 | C | SER | 928 | 0.002 | −30.361 | 11.990 | 1.00 | 11.37 |
| ATOM | 6214 | O | SER | 928 | −0.416 | −30.992 | 11.008 | 1.00 | 11.30 |
| ATOM | 6215 | N | PHE | 929 | −0.786 | −30.004 | 12.997 | 1.00 | 10.21 |
| ATOM | 6216 | CA | PHE | 929 | −2.202 | −30.337 | 12.932 | 1.00 | 9.78 |
| ATOM | 6217 | CB | PHE | 929 | −2.852 | −30.267 | 14.319 | 1.00 | 11.80 |
| ATOM | 6218 | CG | PHE | 929 | −2.628 | −31.504 | 15.132 | 1.00 | 10.16 |
| ATOM | 6219 | CD1 | PHE | 929 | −1.601 | −31.570 | 16.063 | 1.00 | 9.07 |
| ATOM | 6220 | CD2 | PHE | 929 | −3.383 | −32.645 | 14.879 | 1.00 | 13.10 |
| ATOM | 6221 | CE1 | PHE | 929 | −1.325 | −32.766 | 16.737 | 1.00 | 11.64 |
| ATOM | 6222 | CE2 | PHE | 929 | −3.122 | −33.838 | 15.539 | 1.00 | 10.99 |
| ATOM | 6223 | CZ | PHE | 929 | −2.092 | −33.903 | 16.465 | 1.00 | 10.50 |
| ATOM | 6224 | C | PHE | 929 | −2.956 | −29.479 | 11.935 | 1.00 | 10.59 |
| ATOM | 6225 | O | PHE | 929 | −3.871 | −29.970 | 11.280 | 1.00 | 10.20 |
| ATOM | 6226 | N | ALA | 930 | −2.579 | −28.210 | 11.798 | 1.00 | 10.29 |
| ATOM | 6227 | CA | ALA | 930 | −3.263 | −27.341 | 10.835 | 1.00 | 10.49 |
| ATOM | 6228 | CB | ALA | 930 | −2.724 | −25.899 | 10.940 | 1.00 | 10.94 |
| ATOM | 6229 | C | ALA | 930 | −3.028 | −27.891 | 9.427 | 1.00 | 11.83 |
| ATOM | 6230 | O | ALA | 930 | −3.920 | −27.870 | 8.572 | 1.00 | 12.93 |
| ATOM | 6231 | N | LYS | 931 | −1.807 | −28.361 | 9.192 | 1.00 | 11.22 |
| ATOM | 6232 | CA | LYS | 931 | −1.425 | −28.924 | 7.894 | 1.00 | 12.92 |
| ATOM | 6233 | CB | LYS | 931 | 0.078 | −29.232 | 7.897 | 1.00 | 13.90 |
| ATOM | 6234 | CG | LYS | 931 | 0.628 | −29.953 | 6.669 | 1.00 | 19.23 |
| ATOM | 6235 | CD | LYS | 931 | 0.716 | −29.053 | 5.467 | 1.00 | 22.53 |
| ATOM | 6236 | CE | LYS | 931 | 1.677 | −29.628 | 4.415 | 1.00 | 26.66 |
| ATOM | 6237 | NZ | LYS | 931 | 1.255 | −30.976 | 3.915 | 1.00 | 26.76 |
| ATOM | 6238 | C | LYS | 931 | −2.227 | −30.198 | 7.646 | 1.00 | 12.48 |
| ATOM | 6239 | O | LYS | 931 | −2.737 | −30.428 | 6.543 | 1.00 | 14.08 |
| ATOM | 6240 | N | LEU | 932 | −2.326 | −31.029 | 8.672 | 1.00 | 11.21 |
| ATOM | 6241 | CA | LEU | 932 | −3.071 | −32.273 | 8.557 | 1.00 | 11.84 |
| ATOM | 6242 | CB | LEU | 932 | −2.965 | −33.060 | 9.865 | 1.00 | 11.17 |
| ATOM | 6243 | CG | LEU | 932 | −3.550 | −34.485 | 9.854 | 1.00 | 11.63 |
| ATOM | 6244 | CD1 | LEU | 932 | −2.882 | −35.309 | 10.946 | 1.00 | 10.37 |
| ATOM | 6245 | CD2 | LEU | 932 | −5.051 | −34.430 | 10.059 | 1.00 | 12.21 |
| ATOM | 6246 | C | LEU | 932 | −4.534 | −31.991 | 8.212 | 1.00 | 12.85 |
| ATOM | 6247 | O | LEU | 932 | −5.102 | −32.611 | 7.309 | 1.00 | 14.20 |
| ATOM | 6248 | N | PHE | 933 | −5.140 | −31.039 | 8.913 | 1.00 | 12.43 |
| ATOM | 6249 | CA | PHE | 933 | −6.538 | −30.699 | 8.663 | 1.00 | 12.70 |
| ATOM | 6250 | CB | PHE | 933 | −7.036 | −29.670 | 9.691 | 1.00 | 12.30 |
| ATOM | 6251 | CG | PHE | 933 | −6.939 | −30.139 | 11.120 | 1.00 | 10.75 |
| ATOM | 6252 | CD1 | PHE | 933 | −7.001 | −31.493 | 11.430 | 1.00 | 11.83 |
| ATOM | 6253 | CD2 | PHE | 933 | −6.788 | −29.224 | 12.162 | 1.00 | 11.03 |
| ATOM | 6254 | CE1 | PHE | 933 | −6.911 | −31.931 | 12.744 | 1.00 | 11.61 |
| ATOM | 6255 | CE2 | PHE | 933 | −6.699 | −29.662 | 13.482 | 1.00 | 11.00 |
| ATOM | 6256 | CZ | PHE | 933 | −6.758 | −31.014 | 13.774 | 1.00 | 12.40 |
| ATOM | 6257 | C | PHE | 933 | −6.786 | −30.174 | 7.247 | 1.00 | 14.52 |
| ATOM | 6258 | O | PHE | 933 | −7.675 | −30.661 | 6.551 | 1.00 | 14.70 |
| ATOM | 6259 | N | ALA | 934 | −6.010 | −29.181 | 6.822 | 1.00 | 14.17 |
| ATOM | 6260 | CA | ALA | 934 | −6.178 | −28.607 | 5.488 | 1.00 | 16.25 |
| ATOM | 6261 | CB | ALA | 934 | −5.177 | −27.499 | 5.266 | 1.00 | 15.86 |
| ATOM | 6262 | C | ALA | 934 | −6.029 | −29.661 | 4.397 | 1.00 | 18.38 |
| ATOM | 6263 | O | ALA | 934 | −6.742 | −29.631 | 3.399 | 1.00 | 19.61 |
| ATOM | 6264 | N | ASP | 935 | −5.094 | −30.585 | 4.586 | 1.00 | 18.37 |
| ATOM | 6265 | CA | ASP | 935 | −4.873 | −31.632 | 3.605 | 1.00 | 20.68 |
| ATOM | 6266 | CB | ASP | 935 | −3.611 | −32.422 | 3.938 | 1.00 | 22.05 |
| ATOM | 6267 | CG | ASP | 935 | −2.341 | −31.618 | 3.716 | 1.00 | 24.26 |
| ATOM | 6268 | OD1 | ASP | 935 | −2.426 | −30.459 | 3.253 | 1.00 | 28.99 |
| ATOM | 6269 | OD2 | ASP | 935 | −1.256 | −32.155 | 4.001 | 1.00 | 24.81 |
| ATOM | 6270 | C | ASP | 935 | −6.050 | −32.588 | 3.492 | 1.00 | 21.54 |
| ATOM | 6271 | O | ASP | 935 | −6.152 | −33.332 | 2.518 | 1.00 | 21.57 |
| ATOM | 6272 | N | GLU | 936 | −6.937 | −32.570 | 4.481 | 1.00 | 21.31 |
| ATOM | 6273 | CA | GLU | 936 | −8.107 | −33.449 | 4.466 | 1.00 | 23.46 |
| ATOM | 6274 | CB | GLU | 936 | −8.300 | −34.119 | 5.830 | 1.00 | 22.13 |
| ATOM | 6275 | CG | GLU | 936 | −7.196 | −35.073 | 6.234 | 1.00 | 25.30 |
| ATOM | 6276 | CD | GLU | 936 | −6.944 | −36.134 | 5.184 | 1.00 | 26.05 |
| ATOM | 6277 | OE1 | GLU | 936 | −7.932 | −36.697 | 4.664 | 1.00 | 27.53 |
| ATOM | 6278 | OE2 | GLU | 936 | −5.768 | −36.408 | 4.890 | 1.00 | 25.51 |
| ATOM | 6279 | C | GLU | 936 | −9.395 | −32.727 | 4.090 | 1.00 | 23.90 |
| ATOM | 6280 | O | GLU | 936 | −10.433 | −33.367 | 3.928 | 1.00 | 23.71 |
| ATOM | 6281 | N | GLY | 937 | −9.335 | −31.404 | 3.966 | 1.00 | 24.48 |
| ATOM | 6282 | CA | GLY | 937 | −10.527 | −30.654 | 3.603 | 1.00 | 24.58 |
| ATOM | 6283 | C | GLY | 937 | −11.155 | −29.831 | 4.714 | 1.00 | 24.70 |
| ATOM | 6284 | O | GLY | 937 | −12.136 | −29.116 | 4.481 | 1.00 | 22.63 |
| ATOM | 6285 | N | LEU | 938 | −10.614 | −29.942 | 5.925 | 1.00 | 21.96 |
| ATOM | 6286 | CA | LEU | 938 | −11.115 | −29.177 | 7.071 | 1.00 | 22.24 |
| ATOM | 6287 | CB | LEU | 938 | −10.749 | −29.878 | 8.389 | 1.00 | 22.44 |
| ATOM | 6288 | CG | LEU | 938 | −11.881 | −30.305 | 9.325 | 1.00 | 24.89 |
| ATOM | 6289 | CD1 | LEU | 938 | −11.300 | −30.960 | 10.565 | 1.00 | 20.46 |
| ATOM | 6290 | CD2 | LEU | 938 | −12.736 | −29.116 | 9.706 | 1.00 | 23.65 |
| ATOM | 6291 | C | LEU | 938 | −10.397 | −27.839 | 6.979 | 1.00 | 22.22 |
| ATOM | 6292 | O | LEU | 938 | −9.275 | −27.702 | 7.472 | 1.00 | 21.13 |
| ATOM | 6293 | N | ASN | 939 | −11.052 | −26.860 | 6.354 | 1.00 | 19.30 |
| ATOM | 6294 | CA | ASN | 939 | −10.447 | −25.546 | 6.136 | 1.00 | 20.12 |
| ATOM | 6295 | CB | ASN | 939 | −10.577 | −25.159 | 4.657 | 1.00 | 21.00 |
| ATOM | 6296 | CG | ASN | 939 | −10.119 | −26.267 | 3.720 | 1.00 | 24.01 |
| ATOM | 6297 | OD1 | ASN | 939 | −9.005 | −26.783 | 3.846 | 1.00 | 24.14 |
| ATOM | 6298 | ND2 | ASN | 939 | −10.974 | −26.632 | 2.768 | 1.00 | 24.93 |
| ATOM | 6299 | C | ASN | 939 | −10.959 | −24.393 | 7.000 | 1.00 | 17.21 |
| ATOM | 6300 | O | ASN | 939 | −10.813 | −23.234 | 6.631 | 1.00 | 17.54 |
| ATOM | 6301 | N | VAL | 940 | −11.564 | −24.705 | 8.139 | 1.00 | 15.30 |
| ATOM | 6302 | CA | VAL | 940 | −12.038 | −23.664 | 9.048 | 1.00 | 12.98 |
| ATOM | 6303 | CB | VAL | 940 | −13.566 | −23.629 | 9.156 | 1.00 | 13.67 |
| ATOM | 6304 | CG1 | VAL | 940 | −13.986 | −22.479 | 10.070 | 1.00 | 14.67 |
| ATOM | 6305 | CG2 | VAL | 940 | −14.186 | −23.453 | 7.778 | 1.00 | 15.61 |
| ATOM | 6306 | C | VAL | 940 | −11.459 | −24.001 | 10.409 | 1.00 | 12.24 |
| ATOM | 6307 | O | VAL | 940 | −11.854 | −24.987 | 11.032 | 1.00 | 11.95 |
| ATOM | 6308 | N | MET | 941 | −10.525 | −23.182 | 10.877 | 1.00 | 11.66 |
| ATOM | 6309 | CA | MET | 941 | −9.887 | −23.474 | 12.145 | 1.00 | 11.33 |
| ATOM | 6310 | CB | MET | 941 | −8.453 | −23.897 | 11.884 | 1.00 | 11.01 |
| ATOM | 6311 | CG | MET | 941 | −8.355 | −25.076 | 10.935 | 1.00 | 15.14 |
| ATOM | 6312 | SD | MET | 941 | −6.605 | −25.492 | 10.723 | 1.00 | 13.23 |
| ATOM | 6313 | CE | MET | 941 | −6.628 | −25.995 | 9.032 | 1.00 | 16.00 |
| ATOM | 6314 | C | MET | 941 | −9.935 | −22.338 | 13.136 | 1.00 | 11.83 |
| ATOM | 6315 | O | MET | 941 | −9.909 | −21.166 | 12.771 | 1.00 | 12.66 |
| ATOM | 6316 | N | LEU | 942 | −10.020 | −22.710 | 14.404 | 1.00 | 10.99 |
| ATOM | 6317 | CA | LEU | 942 | −10.094 | −21.738 | 15.469 | 1.00 | 12.19 |
| ATOM | 6318 | CB | LEU | 942 | −11.409 | −21.938 | 16.240 | 1.00 | 15.75 |
| ATOM | 6319 | CG | LEU | 942 | −11.850 | −21.101 | 17.453 | 1.00 | 20.87 |
| ATOM | 6320 | CD1 | LEU | 942 | −11.333 | −21.740 | 18.697 | 1.00 | 25.90 |
| ATOM | 6321 | CD2 | LEU | 942 | −11.405 | −19.631 | 17.337 | 1.00 | 18.03 |
| ATOM | 6322 | C | LEU | 942 | −8.903 | −21.821 | 16.416 | 1.00 | 12.22 |
| ATOM | 6323 | O | LEU | 942 | −8.639 | −22.864 | 17.009 | 1.00 | 11.14 |
| ATOM | 6324 | N | VAL | 943 | −8.160 | −20.728 | 16.528 | 1.00 | 11.13 |
| ATOM | 6325 | CA | VAL | 943 | −7.048 | −20.703 | 17.464 | 1.00 | 10.52 |
| ATOM | 6326 | CB | VAL | 943 | −5.889 | −19.821 | 16.965 | 1.00 | 10.69 |
| ATOM | 6327 | CG1 | VAL | 943 | −4.792 | −19.752 | 18.031 | 1.00 | 11.40 |
| ATOM | 6328 | CG2 | VAL | 943 | −5.311 | −20.422 | 15.685 | 1.00 | 13.59 |
| ATOM | 6329 | C | VAL | 943 | −7.721 | −20.095 | 18.690 | 1.00 | 11.08 |
| ATOM | 6330 | O | VAL | 943 | −7.761 | −18.874 | 18.863 | 1.00 | 11.38 |
| ATOM | 6331 | N | GLY | 944 | −8.271 | −20.965 | 19.529 | 1.00 | 13.26 |
| ATOM | 6332 | CA | GLY | 944 | −8.984 | −20.502 | 20.701 | 1.00 | 12.63 |
| ATOM | 6333 | C | GLY | 944 | −8.220 | −20.504 | 22.000 | 1.00 | 11.45 |
| ATOM | 6334 | O | GLY | 944 | −7.168 | −21.149 | 22.120 | 1.00 | 11.27 |
| ATOM | 6335 | N | ASP | 945 | −8.758 | −19.791 | 22.986 | 1.00 | 10.87 |
| ATOM | 6336 | CA | ASP | 945 | −8.094 | −19.714 | 24.277 | 1.00 | 11.19 |
| ATOM | 6337 | CB | ASP | 945 | −8.602 | −18.519 | 25.103 | 1.00 | 12.84 |
| ATOM | 6338 | CG | ASP | 945 | −10.096 | −18.559 | 25.382 | 1.00 | 15.40 |
| ATOM | 6339 | OD1 | ASP | 945 | −10.741 | −19.580 | 25.119 | 1.00 | 14.02 |
| ATOM | 6340 | OD2 | ASP | 945 | −10.612 | −17.544 | 25.888 | 1.00 | 16.56 |
| ATOM | 6341 | C | ASP | 945 | −8.216 | −21.021 | 25.034 | 1.00 | 10.85 |
| ATOM | 6342 | O | ASP | 945 | −7.752 | −21.137 | 26.160 | 1.00 | 11.48 |
| ATOM | 6343 | N | SER | 946 | −8.829 | −22.019 | 24.392 | 1.00 | 10.80 |
| ATOM | 6344 | CA | SER | 946 | −8.929 | −23.346 | 25.000 | 1.00 | 11.18 |
| ATOM | 6345 | CB | SER | 946 | −9.751 | −24.282 | 24.109 | 1.00 | 13.04 |
| ATOM | 6346 | OG | SER | 946 | −9.361 | −24.167 | 22.750 | 1.00 | 13.51 |
| ATOM | 6347 | C | SER | 946 | −7.483 | −23.841 | 25.098 | 1.00 | 12.33 |
| ATOM | 6348 | O | SER | 946 | −7.162 | −24.746 | 25.878 | 1.00 | 12.87 |
| ATOM | 6349 | N | LEU | 947 | −6.608 | −23.241 | 24.292 | 1.00 | 9.71 |
| ATOM | 6350 | CA | LEU | 947 | −5.189 | −23.620 | 24.319 | 1.00 | 10.28 |
| ATOM | 6351 | CB | LEU | 947 | −4.386 | −22.834 | 23.260 | 1.00 | 9.98 |
| ATOM | 6352 | CG | LEU | 947 | −4.241 | −21.308 | 23.369 | 1.00 | 10.69 |
| ATOM | 6353 | CD1 | LEU | 947 | −3.110 | −20.959 | 24.301 | 1.00 | 10.73 |
| ATOM | 6354 | CD2 | LEU | 947 | −3.955 | −20.712 | 21.980 | 1.00 | 11.38 |
| ATOM | 6355 | C | LEU | 947 | −4.607 | −23.391 | 25.717 | 1.00 | 9.28 |
| ATOM | 6356 | O | LEU | 947 | −3.627 | −24.038 | 26.114 | 1.00 | 10.82 |
| ATOM | 6357 | N | GLY | 948 | −5.214 | −22.474 | 26.468 | 1.00 | 9.77 |
| ATOM | 6358 | CA | GLY | 948 | −4.743 | −22.208 | 27.819 | 1.00 | 9.51 |
| ATOM | 6359 | C | GLY | 948 | −4.768 | −23.482 | 28.651 | 1.00 | 10.76 |
| ATOM | 6360 | O | GLY | 948 | −3.966 | −23.647 | 29.570 | 1.00 | 10.19 |
| ATOM | 6361 | N | MET | 949 | −5.684 | −24.389 | 28.325 | 1.00 | 10.94 |
| ATOM | 6362 | CA | MET | 949 | −5.795 | −25.637 | 29.063 | 1.00 | 12.90 |
| ATOM | 6363 | CB | MET | 949 | −7.271 | −25.952 | 29.349 | 1.00 | 12.40 |
| ATOM | 6364 | CG | MET | 949 | −7.987 | −24.849 | 30.130 | 1.00 | 15.92 |
| ATOM | 6365 | SD | MET | 949 | −9.725 | −25.184 | 30.491 | 1.00 | 22.24 |
| ATOM | 6366 | CE | MET | 949 | −9.565 | −26.325 | 31.869 | 1.00 | 23.74 |
| ATOM | 6367 | C | MET | 949 | −5.153 | −26.794 | 28.313 | 1.00 | 13.79 |
| ATOM | 6368 | O | MET | 949 | −4.272 | −27.473 | 28.840 | 1.00 | 13.01 |
| ATOM | 6369 | N | THR | 950 | −5.577 | −27.003 | 27.072 | 1.00 | 13.13 |
| ATOM | 6370 | CA | THR | 950 | −5.061 | −28.104 | 26.271 | 1.00 | 14.84 |
| ATOM | 6371 | CB | THR | 950 | −5.885 | −28.243 | 24.975 | 1.00 | 18.87 |
| ATOM | 6372 | OG1 | THR | 950 | −5.665 | −29.536 | 24.407 | 1.00 | 27.10 |
| ATOM | 6373 | CG2 | THR | 950 | −5.492 | −27.177 | 23.971 | 1.00 | 15.75 |
| ATOM | 6374 | C | THR | 950 | −3.570 | −28.027 | 25.922 | 1.00 | 14.92 |
| ATOM | 6375 | O | THR | 950 | −2.887 | −29.053 | 25.875 | 1.00 | 14.14 |
| ATOM | 6376 | N | VAL | 951 | −3.064 | −26.819 | 25.688 | 1.00 | 12.91 |
| ATOM | 6377 | CA | VAL | 951 | −1.663 | −26.639 | 25.330 | 1.00 | 12.41 |
| ATOM | 6378 | CB | VAL | 951 | −1.532 | −25.597 | 24.174 | 1.00 | 13.11 |
| ATOM | 6379 | CG1 | VAL | 951 | −0.074 | −25.297 | 23.888 | 1.00 | 13.39 |
| ATOM | 6380 | CG2 | VAL | 951 | −2.217 | −26.131 | 22.918 | 1.00 | 13.74 |
| ATOM | 6381 | C | VAL | 951 | −0.769 | −26.213 | 26.502 | 1.00 | 12.91 |
| ATOM | 6382 | O | VAL | 951 | 0.242 | −26.855 | 26.782 | 1.00 | 13.09 |
| ATOM | 6383 | N | GLN | 952 | −1.146 | −25.138 | 27.190 | 1.00 | 10.78 |
| ATOM | 6384 | CA | GLN | 952 | −0.354 | −24.627 | 28.312 | 1.00 | 12.90 |
| ATOM | 6385 | CB | GLN | 952 | −0.668 | −23.149 | 28.540 | 1.00 | 9.47 |
| ATOM | 6386 | CG | GLN | 952 | −0.456 | −22.295 | 27.299 | 1.00 | 10.22 |
| ATOM | 6387 | CD | GLN | 952 | −0.885 | −20.862 | 27.509 | 1.00 | 10.55 |
| ATOM | 6388 | OE1 | GLN | 952 | −1.485 | −20.527 | 28.523 | 1.00 | 11.30 |
| ATOM | 6389 | NE2 | GLN | 952 | −0.571 | −20.006 | 26.547 | 1.00 | 6.63 |
| ATOM | 6390 | C | GLN | 952 | −0.534 | −25.385 | 29.628 | 1.00 | 12.56 |
| ATOM | 6391 | O | GLN | 952 | 0.327 | −25.319 | 30.503 | 1.00 | 14.66 |
| ATOM | 6392 | N | GLY | 953 | −1.667 | −26.061 | 29.791 | 1.00 | 12.38 |
| ATOM | 6393 | CA | GLY | 953 | −1.885 | −26.821 | 31.009 | 1.00 | 12.40 |
| ATOM | 6394 | C | GLY | 953 | −2.460 | −26.083 | 32.206 | 1.00 | 12.68 |
| ATOM | 6395 | O | GLY | 953 | −2.296 | −26.531 | 33.344 | 1.00 | 13.99 |
| ATOM | 6396 | N | HIS | 954 | −3.118 | −24.953 | 31.971 | 1.00 | 12.29 |
| ATOM | 6397 | CA | HIS | 954 | −3.727 | −24.206 | 33.067 | 1.00 | 12.78 |
| ATOM | 6398 | CB | HIS | 954 | −3.889 | −22.731 | 32.700 | 1.00 | 11.26 |
| ATOM | 6399 | CG | HIS | 954 | −2.589 | −22.013 | 32.546 | 1.00 | 13.33 |
| ATOM | 6400 | CD2 | HIS | 954 | −2.013 | −21.430 | 31.470 | 1.00 | 13.37 |
| ATOM | 6401 | ND1 | HIS | 954 | −1.706 | −21.852 | 33.591 | 1.00 | 13.34 |
| ATOM | 6402 | CE1 | HIS | 954 | −0.640 | −21.200 | 33.165 | 1.00 | 15.92 |
| ATOM | 6403 | NE2 | HIS | 954 | −0.801 | −20.931 | 31.882 | 1.00 | 13.61 |
| ATOM | 6404 | C | HIS | 954 | −5.083 | −24.814 | 33.357 | 1.00 | 13.36 |
| ATOM | 6405 | O | HIS | 954 | −5.605 | −25.594 | 32.555 | 1.00 | 13.27 |
| ATOM | 6406 | N | ASP | 955 | −5.644 | −24.458 | 34.511 | 1.00 | 16.24 |
| ATOM | 6407 | CA | ASP | 955 | −6.951 | −24.963 | 34.944 | 1.00 | 18.66 |
| ATOM | 6408 | CB | ASP | 955 | −7.060 | −24.882 | 36.473 | 1.00 | 23.17 |
| ATOM | 6409 | CG | ASP | 955 | −6.943 | −23.457 | 36.998 | 1.00 | 27.78 |
| ATOM | 6410 | OD1 | ASP | 955 | −7.656 | −22.566 | 36.495 | 1.00 | 31.02 |
| ATOM | 6411 | OD2 | ASP | 955 | −6.145 | −23.231 | 37.932 | 1.00 | 32.90 |
| ATOM | 6412 | C | ASP | 955 | −8.131 | −24.221 | 34.315 | 1.00 | 17.97 |
| ATOM | 6413 | O | ASP | 955 | −9.295 | −24.605 | 34.493 | 1.00 | 16.08 |
| ATOM | 6414 | N | SER | 956 | −7.825 | −23.151 | 33.593 | 1.00 | 15.35 |
| ATOM | 6415 | CA | SER | 956 | −8.845 | −22.364 | 32.926 | 1.00 | 14.44 |
| ATOM | 6416 | CB | SER | 956 | −9.444 | −21.320 | 33.878 | 1.00 | 14.03 |
| ATOM | 6417 | OG | SER | 956 | −8.533 | −20.269 | 34.155 | 1.00 | 12.64 |
| ATOM | 6418 | C | SER | 956 | −8.196 | −21.676 | 31.732 | 1.00 | 13.94 |
| ATOM | 6419 | O | SER | 956 | −6.994 | −21.806 | 31.517 | 1.00 | 12.60 |
| ATOM | 6420 | N | THR | 957 | −8.997 | −20.955 | 30.953 | 1.00 | 12.70 |
| ATOM | 6421 | CA | THR | 957 | −8.495 | −20.259 | 29.774 | 1.00 | 11.18 |
| ATOM | 6422 | CB | THR | 957 | −9.579 | −20.165 | 28.690 | 1.00 | 10.23 |
| ATOM | 6423 | OG1 | THR | 957 | −10.710 | −19.468 | 29.217 | 1.00 | 12.66 |
| ATOM | 6424 | CG2 | THR | 957 | −10.017 | −21.566 | 28.231 | 1.00 | 13.69 |
| ATOM | 6425 | C | THR | 957 | −8.047 | −18.834 | 30.114 | 1.00 | 10.60 |
| ATOM | 6426 | O | THR | 957 | −7.415 | −18.164 | 29.295 | 1.00 | 9.48 |
| ATOM | 6427 | N | LEU | 958 | −8.373 | −18.371 | 31.318 | 1.00 | 11.09 |
| ATOM | 6428 | CA | LEU | 958 | −8.042 | −16.999 | 31.718 | 1.00 | 10.38 |
| ATOM | 6429 | CB | LEU | 958 | −8.483 | −16.756 | 33.167 | 1.00 | 11.85 |
| ATOM | 6430 | CG | LEU | 958 | −9.962 | −16.399 | 33.380 | 1.00 | 14.28 |
| ATOM | 6431 | CD1 | LEU | 958 | −10.854 | −17.587 | 33.066 | 1.00 | 13.28 |
| ATOM | 6432 | CD2 | LEU | 958 | −10.159 | −15.961 | 34.824 | 1.00 | 16.63 |
| ATOM | 6433 | C | LEU | 958 | −6.589 | −16.545 | 31.525 | 1.00 | 10.13 |
| ATOM | 6434 | O | LEU | 958 | −6.336 | −15.413 | 31.106 | 1.00 | 10.74 |
| ATOM | 6435 | N | PRO | 959 | −5.611 | −17.417 | 31.819 | 1.00 | 11.82 |
| ATOM | 6436 | CD | PRO | 959 | −5.694 | −18.691 | 32.549 | 1.00 | 12.19 |
| ATOM | 6437 | CA | PRO | 959 | −4.211 | −17.015 | 31.645 | 1.00 | 10.66 |
| ATOM | 6438 | CB | PRO | 959 | −3.437 | −18.170 | 32.284 | 1.00 | 13.42 |
| ATOM | 6439 | CG | PRO | 959 | −4.389 | −19.328 | 32.170 | 1.00 | 19.51 |
| ATOM | 6440 | C | PRO | 959 | −3.722 | −16.711 | 30.225 | 1.00 | 10.61 |
| ATOM | 6441 | O | PRO | 959 | −2.674 | −16.086 | 30.054 | 1.00 | 9.35 |
| ATOM | 6442 | N | VAL | 960 | −4.470 | −17.139 | 29.214 | 1.00 | 10.44 |
| ATOM | 6443 | CA | VAL | 960 | −4.071 | −16.924 | 27.821 | 1.00 | 9.62 |
| ATOM | 6444 | CB | VAL | 960 | −5.046 | −17.647 | 26.858 | 1.00 | 10.31 |
| ATOM | 6445 | CG1 | VAL | 960 | −4.578 | −17.492 | 25.408 | 1.00 | 9.19 |
| ATOM | 6446 | CG2 | VAL | 960 | −5.119 | −19.116 | 27.212 | 1.00 | 10.51 |
| ATOM | 6447 | C | VAL | 960 | −4.017 | −15.455 | 27.455 | 1.00 | 11.03 |
| ATOM | 6448 | O | VAL | 960 | −4.992 | −14.723 | 27.639 | 1.00 | 9.26 |
| ATOM | 6449 | N | THR | 961 | −2.890 | −15.012 | 26.917 | 1.00 | 11.69 |
| ATOM | 6450 | CA | THR | 961 | −2.800 | −13.610 | 26.553 | 1.00 | 12.59 |
| ATOM | 6451 | CB | THR | 961 | −1.512 | −12.975 | 27.142 | 1.00 | 16.60 |
| ATOM | 6452 | OG1 | THR | 961 | −1.351 | −13.392 | 28.510 | 1.00 | 22.57 |
| ATOM | 6453 | CG2 | THR | 961 | −1.587 | −11.461 | 27.095 | 1.00 | 22.84 |
| ATOM | 6454 | C | THR | 961 | −2.854 | −13.423 | 25.040 | 1.00 | 11.62 |
| ATOM | 6455 | O | THR | 961 | −2.785 | −14.393 | 24.272 | 1.00 | 8.55 |
| ATOM | 6456 | N | VAL | 962 | −2.989 | −12.171 | 24.613 | 1.00 | 8.97 |
| ATOM | 6457 | CA | VAL | 962 | −3.059 | −11.870 | 23.189 | 1.00 | 9.43 |
| ATOM | 6458 | CB | VAL | 962 | −3.205 | −10.344 | 22.954 | 1.00 | 12.17 |
| ATOM | 6459 | CG1 | VAL | 962 | −3.243 | −10.044 | 21.457 | 1.00 | 9.65 |
| ATOM | 6460 | CG2 | VAL | 962 | −4.464 | −9.842 | 23.640 | 1.00 | 15.11 |
| ATOM | 6461 | C | VAL | 962 | −1.813 | −12.390 | 22.486 | 1.00 | 10.49 |
| ATOM | 6462 | O | VAL | 962 | −1.902 | −13.023 | 21.434 | 1.00 | 10.98 |
| ATOM | 6463 | N | ALA | 963 | −0.650 | −12.142 | 23.080 | 1.00 | 9.85 |
| ATOM | 6464 | CA | ALA | 963 | 0.602 | −12.604 | 22.489 | 1.00 | 10.83 |
| ATOM | 6465 | CB | ALA | 963 | 1.770 | −12.235 | 23.397 | 1.00 | 10.40 |
| ATOM | 6466 | C | ALA | 963 | 0.564 | −14.120 | 22.258 | 1.00 | 10.40 |
| ATOM | 6467 | O | ALA | 963 | 1.075 | −14.593 | 21.256 | 1.00 | 10.00 |
| ATOM | 6468 | N | ASP | 964 | −0.031 | −14.866 | 23.195 | 1.00 | 12.44 |
| ATOM | 6469 | CA | ASP | 964 | −0.144 | −16.326 | 23.067 | 1.00 | 11.54 |
| ATOM | 6470 | CB | ASP | 964 | −0.818 | −16.961 | 24.289 | 1.00 | 11.14 |
| ATOM | 6471 | CG | ASP | 964 | −0.006 | −16.816 | 25.572 | 1.00 | 13.23 |
| ATOM | 6472 | OD1 | ASP | 964 | 1.239 | −16.781 | 25.528 | 1.00 | 11.81 |
| ATOM | 6473 | OD2 | ASP | 964 | −0.627 | −16.761 | 26.648 | 1.00 | 12.48 |
| ATOM | 6474 | C | ASP | 964 | −0.966 | −16.692 | 21.833 | 1.00 | 11.21 |
| ATOM | 6475 | O | ASP | 964 | −0.583 | −17.561 | 21.048 | 1.00 | 6.94 |
| ATOM | 6476 | N | ILE | 965 | −2.113 | −16.037 | 21.680 | 1.00 | 8.84 |
| ATOM | 6477 | CA | ILE | 965 | −2.978 | −16.292 | 20.539 | 1.00 | 9.08 |
| ATOM | 6478 | CB | ILE | 965 | −4.279 | −15.435 | 20.593 | 1.00 | 9.58 |
| ATOM | 6479 | CG2 | ILE | 965 | −5.067 | −15.617 | 19.298 | 1.00 | 7.50 |
| ATOM | 6480 | CG1 | ILE | 965 | −5.125 | −15.814 | 21.813 | 1.00 | 8.04 |
| ATOM | 6481 | CD1 | ILE | 965 | −5.829 | −17.185 | 21.739 | 1.00 | 9.87 |
| ATOM | 6482 | C | ILE | 965 | −2.237 | −15.970 | 19.244 | 1.00 | 9.13 |
| ATOM | 6483 | O | ILE | 965 | −2.305 | −16.725 | 18.267 | 1.00 | 8.12 |
| ATOM | 6484 | N | ALA | 966 | −1.519 | −14.851 | 19.231 | 1.00 | 9.33 |
| ATOM | 6485 | CA | ALA | 966 | −0.772 | −14.453 | 18.026 | 1.00 | 7.88 |
| ATOM | 6486 | CB | ALA | 966 | −0.161 | −13.046 | 18.215 | 1.00 | 10.51 |
| ATOM | 6487 | C | ALA | 966 | 0.325 | −15.459 | 17.665 | 1.00 | 6.25 |
| ATOM | 6488 | O | ALA | 966 | 0.623 | −15.694 | 16.484 | 1.00 | 8.05 |
| ATOM | 6489 | N | TYR | 967 | 0.954 | −16.023 | 18.690 | 1.00 | 7.24 |
| ATOM | 6490 | CA | TYR | 967 | 2.007 | −17.022 | 18.487 | 1.00 | 7.41 |
| ATOM | 6491 | CB | TYR | 967 | 2.602 | −17.433 | 19.847 | 1.00 | 8.25 |
| ATOM | 6492 | CG | TYR | 967 | 3.602 | −18.579 | 19.775 | 1.00 | 9.15 |
| ATOM | 6493 | CD1 | TYR | 967 | 4.858 | −18.406 | 19.191 | 1.00 | 10.05 |
| ATOM | 6494 | CE1 | TYR | 967 | 5.787 | −19.444 | 19.151 | 1.00 | 12.77 |
| ATOM | 6495 | CD2 | TYR | 967 | 3.296 | −19.826 | 20.314 | 1.00 | 9.48 |
| ATOM | 6496 | CE2 | TYR | 967 | 4.217 | −20.876 | 20.278 | 1.00 | 13.60 |
| ATOM | 6497 | CZ | TYR | 967 | 5.462 | −20.673 | 19.695 | 1.00 | 13.29 |
| ATOM | 6498 | OH | TYR | 967 | 6.394 | −21.697 | 19.655 | 1.00 | 13.05 |
| ATOM | 6499 | C | TYR | 967 | 1.454 | −18.266 | 17.772 | 1.00 | 6.51 |
| ATOM | 6500 | O | TYR | 967 | 2.013 | −18.745 | 16.776 | 1.00 | 5.94 |
| ATOM | 6501 | N | HIS | 968 | 0.347 | −18.783 | 18.296 | 1.00 | 9.58 |
| ATOM | 6502 | CA | HIS | 968 | −0.276 | −19.994 | 17.741 | 1.00 | 9.37 |
| ATOM | 6503 | CB | HIS | 968 | −1.238 | −20.593 | 18.784 | 1.00 | 8.45 |
| ATOM | 6504 | CG | HIS | 968 | −0.533 | −21.162 | 19.988 | 1.00 | 9.03 |
| ATOM | 6505 | CD2 | HIS | 968 | −0.357 | −20.670 | 21.241 | 1.00 | 8.58 |
| ATOM | 6506 | ND1 | HIS | 968 | 0.147 | −22.360 | 19.954 | 1.00 | 8.08 |
| ATOM | 6507 | CE1 | HIS | 968 | 0.714 | −22.582 | 21.129 | 1.00 | 9.22 |
| ATOM | 6508 | NE2 | HIS | 968 | 0.424 | −21.573 | 21.929 | 1.00 | 7.98 |
| ATOM | 6509 | C | HIS | 968 | −0.979 | −19.714 | 16.408 | 1.00 | 10.86 |
| ATOM | 6510 | O | HIS | 968 | −1.057 | −20.590 | 15.536 | 1.00 | 9.61 |
| ATOM | 6511 | N | THR | 969 | −1.468 | −18.486 | 16.237 | 1.00 | 10.09 |
| ATOM | 6512 | CA | THR | 969 | −2.135 | −18.099 | 15.001 | 1.00 | 8.73 |
| ATOM | 6513 | CB | THR | 969 | −2.773 | −16.698 | 15.148 | 1.00 | 11.74 |
| ATOM | 6514 | OG1 | THR | 969 | −3.878 | −16.774 | 16.066 | 1.00 | 12.00 |
| ATOM | 6515 | CG2 | THR | 969 | −3.276 | −16.182 | 13.791 | 1.00 | 10.32 |
| ATOM | 6516 | C | THR | 969 | −1.160 | −18.118 | 13.822 | 1.00 | 10.38 |
| ATOM | 6517 | O | THR | 969 | −1.506 | −18.557 | 12.719 | 1.00 | 10.24 |
| ATOM | 6518 | N | ALA | 970 | 0.064 | −17.653 | 14.056 | 1.00 | 8.37 |
| ATOM | 6519 | CA | ALA | 970 | 1.076 | −17.624 | 13.001 | 1.00 | 9.33 |
| ATOM | 6520 | CB | ALA | 970 | 2.285 | −16.817 | 13.467 | 1.00 | 7.66 |
| ATOM | 6521 | C | ALA | 970 | 1.497 | −19.046 | 12.608 | 1.00 | 10.42 |
| ATOM | 6522 | O | ALA | 970 | 1.805 | −19.311 | 11.445 | 1.00 | 10.11 |
| ATOM | 6523 | N | ALA | 971 | 1.497 | −19.950 | 13.583 | 1.00 | 7.79 |
| ATOM | 6524 | CA | ALA | 971 | 1.876 | −21.345 | 13.352 | 1.00 | 8.64 |
| ATOM | 6525 | CB | ALA | 971 | 2.043 | −22.065 | 14.685 | 1.00 | 10.14 |
| ATOM | 6526 | C | ALA | 971 | 0.808 | −22.037 | 12.520 | 1.00 | 7.88 |
| ATOM | 6527 | O | ALA | 971 | 1.112 | −22.756 | 11.570 | 1.00 | 10.59 |
| ATOM | 6528 | N | VAL | 972 | −0.447 | −21.816 | 12.892 | 1.00 | 9.57 |
| ATOM | 6529 | CA | VAL | 972 | −1.573 | −22.417 | 12.187 | 1.00 | 8.65 |
| ATOM | 6530 | CB | VAL | 972 | −2.901 | −22.072 | 12.910 | 1.00 | 8.10 |
| ATOM | 6531 | CG1 | VAL | 972 | −4.098 | −22.433 | 12.040 | 1.00 | 7.43 |
| ATOM | 6532 | CG2 | VAL | 972 | −2.978 | −22.839 | 14.231 | 1.00 | 10.94 |
| ATOM | 6533 | C | VAL | 972 | −1.604 | −21.935 | 10.740 | 1.00 | 10.50 |
| ATOM | 6534 | O | VAL | 972 | −1.813 | −22.725 | 9.815 | 1.00 | 8.95 |
| ATOM | 6535 | N | ARG | 973 | −1.362 | −20.641 | 10.548 | 1.00 | 10.52 |
| ATOM | 6536 | CA | ARG | 973 | −1.359 | −20.052 | 9.206 | 1.00 | 12.00 |
| ATOM | 6537 | CB | ARG | 973 | −1.179 | −18.526 | 9.291 | 1.00 | 13.02 |
| ATOM | 6538 | CG | ARG | 973 | −1.127 | −17.808 | 7.926 | 1.00 | 14.67 |
| ATOM | 6539 | CD | ARG | 973 | −2.343 | −18.128 | 7.055 | 1.00 | 12.64 |
| ATOM | 6540 | NE | ARG | 973 | −3.543 | −17.405 | 7.457 | 1.00 | 14.38 |
| ATOM | 6541 | CZ | ARG | 973 | −4.783 | −17.779 | 7.149 | 1.00 | 11.66 |
| ATOM | 6542 | NH1 | ARG | 973 | −4.993 | −18.879 | 6.440 | 1.00 | 15.11 |
| ATOM | 6543 | NH2 | ARG | 973 | −5.819 | −17.045 | 7.533 | 1.00 | 13.03 |
| ATOM | 6544 | C | ARG | 973 | −0.260 | −20.666 | 8.349 | 1.00 | 11.81 |
| ATOM | 6545 | O | ARG | 973 | −0.438 | −20.851 | 7.139 | 1.00 | 13.18 |
| ATOM | 6546 | N | ARG | 974 | 0.880 | −20.979 | 8.960 | 1.00 | 10.93 |
| ATOM | 6547 | CA | ARG | 974 | 1.971 | −21.585 | 8.194 | 1.00 | 12.60 |
| ATOM | 6548 | CB | ARG | 974 | 3.251 | −21.701 | 9.033 | 1.00 | 10.96 |
| ATOM | 6549 | CG | ARG | 974 | 3.826 | −20.370 | 9.472 | 1.00 | 13.59 |
| ATOM | 6550 | CD | ARG | 974 | 5.282 | −20.456 | 9.924 | 1.00 | 15.74 |
| ATOM | 6551 | NE | ARG | 974 | 5.711 | −19.147 | 10.394 | 1.00 | 16.85 |
| ATOM | 6552 | CZ | ARG | 974 | 5.655 | −18.750 | 11.659 | 1.00 | 18.61 |
| ATOM | 6553 | NH1 | ARG | 974 | 5.210 | −19.574 | 12.603 | 1.00 | 12.75 |
| ATOM | 6554 | NH2 | ARG | 974 | 5.984 | −17.502 | 11.972 | 1.00 | 19.12 |
| ATOM | 6555 | C | ARG | 974 | 1.562 | −22.967 | 7.707 | 1.00 | 13.55 |
| ATOM | 6556 | O | ARG | 974 | 1.899 | −23.372 | 6.592 | 1.00 | 14.37 |
| ATOM | 6557 | N | GLY | 975 | 0.834 | −23.693 | 8.545 | 1.00 | 13.23 |
| ATOM | 6558 | CA | GLY | 975 | 0.400 | −25.026 | 8.164 | 1.00 | 13.14 |
| ATOM | 6559 | C | GLY | 975 | −0.792 | −25.049 | 7.222 | 1.00 | 12.25 |
| ATOM | 6560 | O | GLY | 975 | −0.957 | −26.002 | 6.462 | 1.00 | 11.97 |
| ATOM | 6561 | N | ALA | 976 | −1.617 | −24.002 | 7.273 | 1.00 | 11.12 |
| ATOM | 6562 | CA | ALA | 976 | −2.817 | −23.896 | 6.441 | 1.00 | 11.30 |
| ATOM | 6563 | CB | ALA | 976 | −4.050 | −24.243 | 7.271 | 1.00 | 10.07 |
| ATOM | 6564 | C | ALA | 976 | −2.962 | −22.480 | 5.863 | 1.00 | 13.51 |
| ATOM | 6565 | O | ALA | 976 | −3.834 | −21.722 | 6.274 | 1.00 | 12.15 |
| ATOM | 6566 | N | PRO | 977 | −2.118 | −22.114 | 4.889 | 1.00 | 14.78 |
| ATOM | 6567 | CD | PRO | 977 | −1.121 | −22.953 | 4.206 | 1.00 | 17.34 |
| ATOM | 6568 | CA | PRO | 977 | −2.179 | −20.773 | 4.288 | 1.00 | 15.20 |
| ATOM | 6569 | CB | PRO | 977 | −1.014 | −20.783 | 3.304 | 1.00 | 15.29 |
| ATOM | 6570 | CG | PRO | 977 | −0.939 | −22.205 | 2.885 | 1.00 | 20.17 |
| ATOM | 6571 | C | PRO | 977 | −3.490 | −20.324 | 3.641 | 1.00 | 15.15 |
| ATOM | 6572 | O | PRO | 977 | −3.727 | −19.121 | 3.486 | 1.00 | 15.71 |
| ATOM | 6573 | N | ASN | 978 | −4.348 | −21.270 | 3.275 | 1.00 | 15.08 |
| ATOM | 6574 | CA | ASN | 978 | −5.616 | −20.924 | 2.631 | 1.00 | 16.97 |
| ATOM | 6575 | CB | ASN | 978 | −5.770 | −21.725 | 1.330 | 1.00 | 21.84 |
| ATOM | 6576 | CG | ASN | 978 | −4.692 | −21.397 | 0.315 | 1.00 | 24.37 |
| ATOM | 6577 | OD1 | ASN | 978 | −4.473 | −20.232 | −0.020 | 1.00 | 26.80 |
| ATOM | 6578 | ND2 | ASN | 978 | −4.018 | −22.423 | −0.185 | 1.00 | 28.38 |
| ATOM | 6579 | C | ASN | 978 | −6.856 | −21.137 | 3.497 | 1.00 | 15.81 |
| ATOM | 6580 | O | ASN | 978 | −7.978 | −20.909 | 3.046 | 1.00 | 16.34 |
| ATOM | 6581 | N | CYS | 979 | −6.671 | −21.541 | 4.745 | 1.00 | 15.45 |
| ATOM | 6582 | CA | CYS | 979 | −7.828 | −21.787 | 5.597 | 1.00 | 15.35 |
| ATOM | 6583 | CB | CYS | 979 | −7.427 | −22.656 | 6.803 | 1.00 | 15.73 |
| ATOM | 6584 | SG | CYS | 979 | −6.617 | −21.757 | 8.193 | 1.00 | 17.53 |
| ATOM | 6585 | C | CYS | 979 | −8.469 | −20.495 | 6.096 | 1.00 | 13.52 |
| ATOM | 6586 | O | CYS | 979 | −7.869 | −19.416 | 6.035 | 1.00 | 15.76 |
| ATOM | 6587 | N | LEU | 980 | −9.709 | −20.603 | 6.559 | 1.00 | 13.60 |
| ATOM | 6588 | CA | LEU | 980 | −10.396 | −19.458 | 7.148 | 1.00 | 11.99 |
| ATOM | 6589 | CB | LEU | 980 | −11.914 | −19.582 | 7.017 | 1.00 | 13.15 |
| ATOM | 6590 | CG | LEU | 980 | −12.747 | −18.553 | 7.795 | 1.00 | 13.09 |
| ATOM | 6591 | CD1 | LEU | 980 | −12.439 | −17.144 | 7.313 | 1.00 | 17.24 |
| ATOM | 6592 | CD2 | LEU | 980 | −14.229 | −18.871 | 7.628 | 1.00 | 13.80 |
| ATOM | 6593 | C | LEU | 980 | −9.970 | −19.627 | 8.604 | 1.00 | 11.62 |
| ATOM | 6594 | O | LEU | 980 | −10.336 | −20.601 | 9.262 | 1.00 | 11.81 |
| ATOM | 6595 | N | LEU | 981 | −9.187 | −18.677 | 9.099 | 1.00 | 12.13 |
| ATOM | 6596 | CA | LEU | 981 | −8.647 | −18.760 | 10.447 | 1.00 | 9.55 |
| ATOM | 6597 | CB | LEU | 981 | −7.132 | −18.495 | 10.387 | 1.00 | 10.99 |
| ATOM | 6598 | CG | LEU | 981 | −6.195 | −18.853 | 11.539 | 1.00 | 12.12 |
| ATOM | 6599 | CD1 | LEU | 981 | −4.741 | −18.706 | 11.090 | 1.00 | 12.07 |
| ATOM | 6600 | CD2 | LEU | 981 | −6.472 | −17.977 | 12.747 | 1.00 | 15.27 |
| ATOM | 6601 | C | LEU | 981 | −9.301 | −17.827 | 11.448 | 1.00 | 10.37 |
| ATOM | 6602 | O | LEU | 981 | −9.197 | −16.604 | 11.321 | 1.00 | 11.18 |
| ATOM | 6603 | N | LEU | 982 | −9.984 | −18.394 | 12.445 | 1.00 | 9.54 |
| ATOM | 6604 | CA | LEU | 982 | −10.604 | −17.564 | 13.485 | 1.00 | 10.87 |
| ATOM | 6605 | CB | LEU | 982 | −11.948 | −18.140 | 13.951 | 1.00 | 12.12 |
| ATOM | 6606 | CG | LEU | 982 | −13.182 | −17.868 | 13.095 | 1.00 | 13.63 |
| ATOM | 6607 | CD1 | LEU | 982 | −12.997 | −18.480 | 11.715 | 1.00 | 13.85 |
| ATOM | 6608 | CD2 | LEU | 982 | −14.414 | −18.442 | 13.792 | 1.00 | 14.38 |
| ATOM | 6609 | C | LEU | 982 | −9.659 | −17.532 | 14.675 | 1.00 | 12.49 |
| ATOM | 6610 | O | LEU | 982 | −8.991 | −18.521 | 14.951 | 1.00 | 12.63 |
| ATOM | 6611 | N | ALA | 983 | −9.579 | −16.400 | 15.364 | 1.00 | 11.23 |
| ATOM | 6612 | CA | ALA | 983 | −8.715 | −16.324 | 16.533 | 1.00 | 11.28 |
| ATOM | 6613 | CB | ALA | 983 | −7.440 | −15.542 | 16.217 | 1.00 | 9.62 |
| ATOM | 6614 | C | ALA | 983 | −9.455 | −15.676 | 17.682 | 1.00 | 9.95 |
| ATOM | 6615 | O | ALA | 983 | −10.043 | −14.601 | 17.537 | 1.00 | 11.64 |
| ATOM | 6616 | N | ASP | 984 | −9.452 | −16.347 | 18.829 | 1.00 | 10.15 |
| ATOM | 6617 | CA | ASP | 984 | −10.116 | −15.825 | 20.026 | 1.00 | 11.00 |
| ATOM | 6618 | CB | ASP | 984 | −10.108 | −16.871 | 21.148 | 1.00 | 14.08 |
| ATOM | 6619 | CG | ASP | 984 | −11.332 | −17.763 | 21.159 | 1.00 | 16.37 |
| ATOM | 6620 | OD1 | ASP | 984 | −12.305 | −17.478 | 20.438 | 1.00 | 14.80 |
| ATOM | 6621 | OD2 | ASP | 984 | −11.304 | −18.755 | 21.923 | 1.00 | 14.64 |
| ATOM | 6622 | C | ASP | 984 | −9.390 | −14.627 | 20.592 | 1.00 | 10.62 |
| ATOM | 6623 | O | ASP | 984 | −8.161 | −14.538 | 20.524 | 1.00 | 10.17 |
| ATOM | 6624 | N | LEU | 985 | −10.151 | −13.688 | 21.137 | 1.00 | 10.79 |
| ATOM | 6625 | CA | LEU | 985 | −9.529 | −12.587 | 21.864 | 1.00 | 11.87 |
| ATOM | 6626 | CB | LEU | 985 | −10.323 | −11.295 | 21.763 | 1.00 | 12.83 |
| ATOM | 6627 | CG | LEU | 985 | −10.091 | −10.510 | 20.479 | 1.00 | 14.09 |
| ATOM | 6628 | CD1 | LEU | 985 | −10.537 | −9.065 | 20.695 | 1.00 | 16.64 |
| ATOM | 6629 | CD2 | LEU | 985 | −8.613 | −10.542 | 20.102 | 1.00 | 17.01 |
| ATOM | 6630 | C | LEU | 985 | −9.725 | −13.204 | 23.247 | 1.00 | 11.19 |
| ATOM | 6631 | O | LEU | 985 | −10.840 | −13.590 | 23.606 | 1.00 | 12.15 |
| ATOM | 6632 | N | PRO | 986 | −8.651 | −13.315 | 24.034 | 1.00 | 10.32 |
| ATOM | 6633 | CD | PRO | 986 | −7.280 | −12.891 | 23.695 | 1.00 | 10.52 |
| ATOM | 6634 | CA | PRO | 986 | −8.698 | −13.906 | 25.372 | 1.00 | 11.35 |
| ATOM | 6635 | CB | PRO | 986 | −7.238 | −14.221 | 25.633 | 1.00 | 12.21 |
| ATOM | 6636 | CG | PRO | 986 | −6.563 | −13.023 | 25.015 | 1.00 | 12.75 |
| ATOM | 6637 | C | PRO | 986 | −9.309 | −13.046 | 26.482 | 1.00 | 10.95 |
| ATOM | 6638 | O | PRO | 986 | −9.721 | −11.914 | 26.255 | 1.00 | 10.33 |
| ATOM | 6639 | N | PHE | 987 | −9.364 | −13.614 | 27.684 | 1.00 | 9.49 |
| ATOM | 6640 | CA | PHE | 987 | −9.913 | −12.938 | 28.856 | 1.00 | 10.31 |
| ATOM | 6641 | CB | PHE | 987 | −9.524 | −13.722 | 30.118 | 1.00 | 10.30 |
| ATOM | 6642 | CG | PHE | 987 | −9.770 | −12.984 | 31.418 | 1.00 | 11.95 |
| ATOM | 6643 | CD1 | PHE | 987 | −11.054 | −12.630 | 31.821 | 1.00 | 12.08 |
| ATOM | 6644 | CD2 | PHE | 987 | −8.705 | −12.697 | 32.265 | 1.00 | 11.62 |
| ATOM | 6645 | CE1 | PHE | 987 | −11.272 | −11.999 | 33.054 | 1.00 | 13.41 |
| ATOM | 6646 | CE2 | PHE | 987 | −8.910 | −12.070 | 33.493 | 1.00 | 12.09 |
| ATOM | 6647 | CZ | PHE | 987 | −10.196 | −11.724 | 33.885 | 1.00 | 13.88 |
| ATOM | 6648 | C | PHE | 987 | −9.421 | −11.491 | 28.955 | 1.00 | 11.22 |
| ATOM | 6649 | O | PHE | 987 | −8.223 | −11.220 | 28.861 | 1.00 | 9.94 |
| ATOM | 6650 | N | MET | 988 | −10.371 | −10.579 | 29.136 | 1.00 | 10.88 |
| ATOM | 6651 | CA | MET | 988 | −10.118 | −9.153 | 29.274 | 1.00 | 12.00 |
| ATOM | 6652 | CB | MET | 988 | −9.447 | −8.854 | 30.630 | 1.00 | 11.75 |
| ATOM | 6653 | CG | MET | 988 | −9.721 | −7.436 | 31.166 | 1.00 | 13.31 |
| ATOM | 6654 | SD | MET | 988 | −11.475 | −7.116 | 31.566 | 1.00 | 13.55 |
| ATOM | 6655 | CE | MET | 988 | −11.538 | −7.686 | 33.263 | 1.00 | 18.85 |
| ATOM | 6656 | C | MET | 988 | −9.300 | −8.512 | 28.159 | 1.00 | 13.38 |
| ATOM | 6657 | O | MET | 988 | −8.568 | −7.556 | 28.411 | 1.00 | 16.30 |
| ATOM | 6658 | N | ALA | 989 | −9.420 | −9.019 | 26.937 | 1.00 | 11.97 |
| ATOM | 6659 | CA | ALA | 989 | −8.681 | −8.450 | 25.816 | 1.00 | 12.87 |
| ATOM | 6660 | CB | ALA | 989 | −8.073 | −9.568 | 24.953 | 1.00 | 11.39 |
| ATOM | 6661 | C | ALA | 989 | −9.585 | −7.554 | 24.959 | 1.00 | 12.13 |
| ATOM | 6662 | O | ALA | 989 | −9.158 | −7.019 | 23.938 | 1.00 | 14.24 |
| ATOM | 6663 | N | TYR | 990 | −10.833 | −7.391 | 25.389 | 1.00 | 11.11 |
| ATOM | 6664 | CA | TYR | 990 | −11.786 | −6.538 | 24.694 | 1.00 | 11.34 |
| ATOM | 6665 | CB | TYR | 990 | −12.591 | −7.352 | 23.660 | 1.00 | 11.56 |
| ATOM | 6666 | CG | TYR | 990 | −13.140 | −8.668 | 24.180 | 1.00 | 14.52 |
| ATOM | 6667 | CD1 | TYR | 990 | −14.464 | −8.776 | 24.593 | 1.00 | 11.96 |
| ATOM | 6668 | CE1 | TYR | 990 | −14.979 | −9.979 | 25.082 | 1.00 | 14.62 |
| ATOM | 6669 | CD2 | TYR | 990 | −12.327 | −9.802 | 24.267 | 1.00 | 12.94 |
| ATOM | 6670 | CE2 | TYR | 990 | −12.825 | −11.017 | 24.759 | 1.00 | 14.62 |
| ATOM | 6671 | CZ | TYR | 990 | −14.156 | −11.091 | 25.163 | 1.00 | 15.49 |
| ATOM | 6672 | OH | TYR | 990 | −14.675 | −12.270 | 25.655 | 1.00 | 18.21 |
| ATOM | 6673 | C | TYR | 990 | −12.706 | −5.851 | 25.711 | 1.00 | 12.49 |
| ATOM | 6674 | O | TYR | 990 | −13.906 | −5.689 | 25.481 | 1.00 | 14.32 |
| ATOM | 6675 | N | ALA | 991 | −12.116 | −5.430 | 26.827 | 1.00 | 12.73 |
| ATOM | 6676 | CA | ALA | 991 | −12.844 | −4.770 | 27.913 | 1.00 | 12.56 |
| ATOM | 6677 | CB | ALA | 991 | −11.927 | −4.561 | 29.104 | 1.00 | 11.96 |
| ATOM | 6678 | C | ALA | 991 | −13.424 | −3.434 | 27.478 | 1.00 | 12.37 |
| ATOM | 6679 | O | ALA | 991 | −14.410 | −2.963 | 28.044 | 1.00 | 12.98 |
| ATOM | 6680 | N | THR | 992 | −12.779 | −2.803 | 26.505 | 1.00 | 13.27 |
| ATOM | 6681 | CA | THR | 992 | −13.248 | −1.527 | 25.967 | 1.00 | 13.52 |
| ATOM | 6682 | CB | THR | 992 | −12.390 | −0.333 | 26.462 | 1.00 | 15.27 |
| ATOM | 6683 | OG1 | THR | 992 | −11.069 | −0.437 | 25.918 | 1.00 | 15.35 |
| ATOM | 6684 | CG2 | THR | 992 | −12.286 | −0.326 | 27.983 | 1.00 | 15.49 |
| ATOM | 6685 | C | THR | 992 | −13.093 | −1.640 | 24.454 | 1.00 | 15.81 |
| ATOM | 6686 | O | THR | 992 | −12.316 | −2.463 | 23.965 | 1.00 | 11.88 |
| ATOM | 6687 | N | PRO | 993 | −13.849 | −0.839 | 23.690 | 1.00 | 14.26 |
| ATOM | 6688 | CD | PRO | 993 | −15.022 | −0.033 | 24.082 | 1.00 | 14.65 |
| ATOM | 6689 | CA | PRO | 993 | −13.723 | −0.912 | 22.233 | 1.00 | 15.27 |
| ATOM | 6690 | CB | PRO | 993 | −14.663 | 0.189 | 21.756 | 1.00 | 13.69 |
| ATOM | 6691 | CG | PRO | 993 | −15.781 | 0.103 | 22.771 | 1.00 | 14.48 |
| ATOM | 6692 | C | PRO | 993 | −12.279 | −0.675 | 21.794 | 1.00 | 15.17 |
| ATOM | 6693 | O | PRO | 993 | −11.745 | −1.403 | 20.955 | 1.00 | 14.48 |
| ATOM | 6694 | N | GLU | 994 | −11.656 | 0.343 | 22.383 | 1.00 | 14.90 |
| ATOM | 6695 | CA | GLU | 994 | −10.284 | 0.714 | 22.068 | 1.00 | 16.61 |
| ATOM | 6696 | CB | GLU | 994 | −9.847 | 1.881 | 22.951 | 1.00 | 20.94 |
| ATOM | 6697 | CG | GLU | 994 | −8.673 | 2.652 | 22.415 | 1.00 | 29.96 |
| ATOM | 6698 | CD | GLU | 994 | −8.335 | 3.857 | 23.277 | 1.00 | 32.49 |
| ATOM | 6699 | OE1 | GLU | 994 | −7.498 | 3.719 | 24.199 | 1.00 | 35.55 |
| ATOM | 6700 | OE2 | GLU | 994 | −8.923 | 4.934 | 23.038 | 1.00 | 33.67 |
| ATOM | 6701 | C | GLU | 994 | −9.313 | −0.447 | 22.256 | 1.00 | 15.95 |
| ATOM | 6702 | O | GLU | 994 | −8.439 | −0.675 | 21.421 | 1.00 | 14.70 |
| ATOM | 6703 | N | GLN | 995 | −9.455 | −1.170 | 23.361 | 1.00 | 14.74 |
| ATOM | 6704 | CA | GLN | 995 | −8.586 | −2.307 | 23.625 | 1.00 | 14.57 |
| ATOM | 6705 | CB | GLN | 995 | −8.758 | −2.793 | 25.058 | 1.00 | 15.85 |
| ATOM | 6706 | CG | GLN | 995 | −8.325 | −1.765 | 26.084 | 1.00 | 22.47 |
| ATOM | 6707 | CD | GLN | 995 | −8.360 | −2.315 | 27.480 | 1.00 | 26.05 |
| ATOM | 6708 | OE1 | GLN | 995 | −7.468 | −3.057 | 27.886 | 1.00 | 27.48 |
| ATOM | 6709 | NE2 | GLN | 995 | −9.414 | −1.980 | 28.225 | 1.00 | 29.12 |
| ATOM | 6710 | C | GLN | 995 | −8.892 | −3.433 | 22.664 | 1.00 | 11.23 |
| ATOM | 6711 | O | GLN | 995 | −7.992 | −4.142 | 22.222 | 1.00 | 12.90 |
| ATOM | 6712 | N | ALA | 996 | −10.170 | −3.612 | 22.361 | 1.00 | 11.62 |
| ATOM | 6713 | CA | ALA | 996 | −10.578 | −4.647 | 21.422 | 1.00 | 10.29 |
| ATOM | 6714 | CB | ALA | 996 | −12.103 | −4.647 | 21.266 | 1.00 | 12.23 |
| ATOM | 6715 | C | ALA | 996 | −9.917 | −4.396 | 20.069 | 1.00 | 11.82 |
| ATOM | 6716 | O | ALA | 996 | −9.402 | −5.321 | 19.431 | 1.00 | 11.66 |
| ATOM | 6717 | N | PHE | 997 | −9.933 | −3.141 | 19.621 | 1.00 | 11.85 |
| ATOM | 6718 | CA | PHE | 997 | −9.325 | −2.808 | 18.327 | 1.00 | 13.01 |
| ATOM | 6719 | CB | PHE | 997 | −9.423 | −1.308 | 18.025 | 1.00 | 12.43 |
| ATOM | 6720 | CG | PHE | 997 | −10.813 | −0.747 | 18.100 | 1.00 | 12.02 |
| ATOM | 6721 | CD1 | PHE | 997 | −11.921 | −1.528 | 17.811 | 1.00 | 14.72 |
| ATOM | 6722 | CD2 | PHE | 997 | −11.001 | 0.588 | 18.443 | 1.00 | 15.95 |
| ATOM | 6723 | CE1 | PHE | 997 | −13.209 | −0.992 | 17.865 | 1.00 | 16.49 |
| ATOM | 6724 | CE2 | PHE | 997 | −12.279 | 1.140 | 18.499 | 1.00 | 13.96 |
| ATOM | 6725 | CZ | PHE | 997 | −13.379 | 0.344 | 18.211 | 1.00 | 17.62 |
| ATOM | 6726 | C | PHE | 997 | −7.846 | −3.179 | 18.300 | 1.00 | 13.37 |
| ATOM | 6727 | O | PHE | 997 | −7.364 | −3.784 | 17.343 | 1.00 | 13.24 |
| ATOM | 6728 | N | GLU | 998 | −7.128 | −2.779 | 19.341 | 1.00 | 13.15 |
| ATOM | 6729 | CA | GLU | 998 | −5.701 | −3.054 | 19.430 | 1.00 | 15.16 |
| ATOM | 6730 | CB | GLU | 998 | −5.123 | −2.380 | 20.674 | 1.00 | 17.72 |
| ATOM | 6731 | CG | GLU | 998 | −3.669 | −2.700 | 20.952 | 1.00 | 24.55 |
| ATOM | 6732 | CD | GLU | 998 | −2.709 | −1.972 | 20.023 | 1.00 | 28.59 |
| ATOM | 6733 | OE1 | GLU | 998 | −3.168 | −1.301 | 19.068 | 1.00 | 30.41 |
| ATOM | 6734 | OE2 | GLU | 998 | −1.486 | −2.077 | 20.254 | 1.00 | 31.76 |
| ATOM | 6735 | C | GLU | 998 | −5.389 | −4.554 | 19.461 | 1.00 | 12.56 |
| ATOM | 6736 | O | GLU | 998 | −4.523 | −5.029 | 18.730 | 1.00 | 10.73 |
| ATOM | 6737 | N | ASN | 999 | −6.093 | −5.303 | 20.301 | 1.00 | 12.22 |
| ATOM | 6738 | CA | ASN | 999 | −5.833 | −6.729 | 20.393 | 1.00 | 12.09 |
| ATOM | 6739 | CB | ASN | 999 | −6.429 | −7.280 | 21.694 | 1.00 | 11.99 |
| ATOM | 6740 | CG | ASN | 999 | −5.719 | −6.735 | 22.912 | 1.00 | 14.10 |
| ATOM | 6741 | OD1 | ASN | 999 | −4.503 | −6.514 | 22.869 | 1.00 | 13.95 |
| ATOM | 6742 | ND2 | ASN | 999 | −6.456 | −6.520 | 24.010 | 1.00 | 13.68 |
| ATOM | 6743 | C | ASN | 999 | −6.304 | −7.523 | 19.182 | 1.00 | 12.22 |
| ATOM | 6744 | O | ASN | 999 | −5.667 | −8.504 | 18.775 | 1.00 | 10.24 |
| ATOM | 6745 | N | ALA | 1000 | −7.411 | −7.101 | 18.589 | 1.00 | 11.49 |
| ATOM | 6746 | CA | ALA | 1000 | −7.912 | −7.777 | 17.399 | 1.00 | 12.27 |
| ATOM | 6747 | CB | ALA | 1000 | −9.280 | −7.201 | 16.998 | 1.00 | 11.29 |
| ATOM | 6748 | C | ALA | 1000 | −6.894 | −7.571 | 16.265 | 1.00 | 12.84 |
| ATOM | 6749 | O | ALA | 1000 | −6.617 | −8.488 | 15.493 | 1.00 | 11.51 |
| ATOM | 6750 | N | ALA | 1001 | −6.325 | −6.371 | 16.174 | 1.00 | 12.62 |
| ATOM | 6751 | CA | ALA | 1001 | −5.340 | −6.094 | 15.128 | 1.00 | 12.98 |
| ATOM | 6752 | CB | ALA | 1001 | −4.941 | −4.619 | 15.165 | 1.00 | 11.69 |
| ATOM | 6753 | C | ALA | 1001 | −4.107 | −6.978 | 15.300 | 1.00 | 11.52 |
| ATOM | 6754 | O | ALA | 1001 | −3.545 | −7.477 | 14.324 | 1.00 | 11.98 |
| ATOM | 6755 | N | THR | 1002 | −3.681 | −7.165 | 16.542 | 1.00 | 10.66 |
| ATOM | 6756 | CA | THR | 1002 | −2.515 | −8.007 | 16.804 | 1.00 | 11.02 |
| ATOM | 6757 | CB | THR | 1002 | −2.228 | −8.117 | 18.319 | 1.00 | 11.27 |
| ATOM | 6758 | OG1 | THR | 1002 | −1.930 | −6.813 | 18.841 | 1.00 | 12.88 |
| ATOM | 6759 | CG2 | THR | 1002 | −1.036 | −9.040 | 18.584 | 1.00 | 11.56 |
| ATOM | 6760 | C | THR | 1002 | −2.739 | −9.411 | 16.246 | 1.00 | 10.94 |
| ATOM | 6761 | O | THR | 1002 | −1.897 | −9.952 | 15.523 | 1.00 | 11.20 |
| ATOM | 6762 | N | VAL | 1003 | −3.881 | −10.005 | 16.563 | 1.00 | 9.24 |
| ATOM | 6763 | CA | VAL | 1003 | −4.154 | −11.372 | 16.102 | 1.00 | 9.83 |
| ATOM | 6764 | CB | VAL | 1003 | −5.342 | −11.991 | 16.897 | 1.00 | 11.74 |
| ATOM | 6765 | CG1 | VAL | 1003 | −6.680 | −11.511 | 16.373 | 1.00 | 10.79 |
| ATOM | 6766 | CG2 | VAL | 1003 | −5.231 | −13.484 | 16.875 | 1.00 | 19.69 |
| ATOM | 6767 | C | VAL | 1003 | −4.374 | −11.466 | 14.593 | 1.00 | 10.01 |
| ATOM | 6768 | O | VAL | 1003 | −3.999 | −12.462 | 13.965 | 1.00 | 7.78 |
| ATOM | 6769 | N | MET | 1004 | −4.967 | −10.423 | 14.013 | 1.00 | 8.67 |
| ATOM | 6770 | CA | MET | 1004 | −5.205 | −10.368 | 12.570 | 1.00 | 11.82 |
| ATOM | 6771 | CB | MET | 1004 | −6.107 | −9.174 | 12.213 | 1.00 | 11.14 |
| ATOM | 6772 | CG | MET | 1004 | −7.523 | −9.240 | 12.728 | 1.00 | 16.34 |
| ATOM | 6773 | SD | MET | 1004 | −8.546 | −10.464 | 11.934 | 1.00 | 19.40 |
| ATOM | 6774 | CE | MET | 1004 | −8.653 | −9.772 | 10.242 | 1.00 | 17.67 |
| ATOM | 6775 | C | MET | 1004 | −3.878 | −10.211 | 11.825 | 1.00 | 10.89 |
| ATOM | 6776 | O | MET | 1004 | −3.665 | −10.860 | 10.807 | 1.00 | 13.67 |
| ATOM | 6777 | N | ARG | 1005 | −2.988 | −9.345 | 12.311 | 1.00 | 11.14 |
| ATOM | 6778 | CA | ARG | 1005 | −1.708 | −9.184 | 11.619 | 1.00 | 11.02 |
| ATOM | 6779 | CB | ARG | 1005 | −0.862 | −8.066 | 12.234 | 1.00 | 12.00 |
| ATOM | 6780 | CG | ARG | 1005 | −1.490 | −6.689 | 12.114 | 1.00 | 16.37 |
| ATOM | 6781 | CD | ARG | 1005 | −0.472 | −5.584 | 12.356 | 1.00 | 15.62 |
| ATOM | 6782 | NE | ARG | 1005 | −1.134 | −4.292 | 12.558 | 1.00 | 17.19 |
| ATOM | 6783 | CZ | ARG | 1005 | −1.491 | −3.811 | 13.744 | 1.00 | 16.30 |
| ATOM | 6784 | NH1 | ARG | 1005 | −1.248 | −4.501 | 14.854 | 1.00 | 16.09 |
| ATOM | 6785 | NH2 | ARG | 1005 | −2.110 | −2.646 | 13.818 | 1.00 | 17.74 |
| ATOM | 6786 | C | ARG | 1005 | −0.916 | −10.491 | 11.659 | 1.00 | 11.70 |
| ATOM | 6787 | O | ARG | 1005 | −0.139 | −10.787 | 10.750 | 1.00 | 11.26 |
| ATOM | 6788 | N | ALA | 1006 | −1.142 | −11.266 | 12.716 | 1.00 | 11.42 |
| ATOM | 6789 | CA | ALA | 1006 | −0.475 | −12.540 | 12.918 | 1.00 | 11.69 |
| ATOM | 6790 | CB | ALA | 1006 | −0.660 | −12.996 | 14.363 | 1.00 | 12.29 |
| ATOM | 6791 | C | ALA | 1006 | −0.955 | −13.639 | 11.963 | 1.00 | 14.06 |
| ATOM | 6792 | O | ALA | 1006 | −0.333 | −14.703 | 11.864 | 1.00 | 13.17 |
| ATOM | 6793 | N | GLY | 1007 | −2.068 | −13.399 | 11.280 | 1.00 | 11.67 |
| ATOM | 6794 | CA | GLY | 1007 | −2.558 | −14.384 | 10.336 | 1.00 | 13.30 |
| ATOM | 6795 | C | GLY | 1007 | −4.043 | −14.701 | 10.358 | 1.00 | 11.83 |
| ATOM | 6796 | O | GLY | 1007 | −4.546 | −15.400 | 9.474 | 1.00 | 13.69 |
| ATOM | 6797 | N | ALA | 1008 | −4.750 | −14.183 | 11.354 | 1.00 | 10.96 |
| ATOM | 6798 | CA | ALA | 1008 | −6.177 | −14.430 | 11.484 | 1.00 | 10.68 |
| ATOM | 6799 | CB | ALA | 1008 | −6.641 | −14.059 | 12.894 | 1.00 | 11.65 |
| ATOM | 6800 | C | ALA | 1008 | −7.010 | −13.665 | 10.453 | 1.00 | 13.14 |
| ATOM | 6801 | O | ALA | 1008 | −6.591 | −12.624 | 9.951 | 1.00 | 14.60 |
| ATOM | 6802 | N | ASN | 1009 | −8.193 | −14.192 | 10.151 | 1.00 | 12.15 |
| ATOM | 6803 | CA | ASN | 1009 | −9.115 | −13.567 | 9.200 | 1.00 | 11.79 |
| ATOM | 6804 | CB | ASN | 1009 | −9.652 | −14.579 | 8.197 | 1.00 | 13.02 |
| ATOM | 6805 | CG | ASN | 1009 | −8.583 | −15.225 | 7.376 | 1.00 | 13.44 |
| ATOM | 6806 | OD1 | ASN | 1009 | −7.839 | −14.559 | 6.664 | 1.00 | 15.40 |
| ATOM | 6807 | ND2 | ASN | 1009 | −8.507 | −16.550 | 7.457 | 1.00 | 10.54 |
| ATOM | 6808 | C | ASN | 1009 | −10.327 | −13.057 | 9.954 | 1.00 | 11.95 |
| ATOM | 6809 | O | ASN | 1009 | −11.119 | −12.276 | 9.429 | 1.00 | 11.36 |
| ATOM | 6810 | N | MET | 1010 | −10.493 | −13.536 | 11.177 | 1.00 | 11.61 |
| ATOM | 6811 | CA | MET | 1010 | −11.660 | −13.165 | 11.956 | 1.00 | 12.67 |
| ATOM | 6812 | CB | MET | 1010 | −12.824 | −14.052 | 11.513 | 1.00 | 10.94 |
| ATOM | 6813 | CG | MET | 1010 | −14.101 | −13.892 | 12.297 | 1.00 | 16.45 |
| ATOM | 6814 | SD | MET | 1010 | −15.423 | −14.958 | 11.623 | 1.00 | 18.40 |
| ATOM | 6815 | CE | MET | 1010 | −16.334 | −13.742 | 10.618 | 1.00 | 17.91 |
| ATOM | 6816 | C | MET | 1010 | −11.357 | −13.364 | 13.431 | 1.00 | 12.79 |
| ATOM | 6817 | O | MET | 1010 | −10.541 | −14.207 | 13.792 | 1.00 | 10.90 |
| ATOM | 6818 | N | VAL | 1011 | −12.005 | −12.571 | 14.276 | 1.00 | 11.92 |
| ATOM | 6819 | CA | VAL | 1011 | −11.787 | −12.664 | 15.701 | 1.00 | 12.96 |
| ATOM | 6820 | CB | VAL | 1011 | −11.327 | −11.300 | 16.246 | 1.00 | 14.64 |
| ATOM | 6821 | CG1 | VAL | 1011 | −11.267 | −11.331 | 17.739 | 1.00 | 21.61 |
| ATOM | 6822 | CG2 | VAL | 1011 | −9.950 | −10.951 | 15.668 | 1.00 | 14.08 |
| ATOM | 6823 | C | VAL | 1011 | −13.064 | −13.121 | 16.397 | 1.00 | 11.60 |
| ATOM | 6824 | O | VAL | 1011 | −14.165 | −12.769 | 15.978 | 1.00 | 11.44 |
| ATOM | 6825 | N | LYS | 1012 | −12.924 | −13.940 | 17.436 | 1.00 | 11.24 |
| ATOM | 6826 | CA | LYS | 1012 | −14.088 | −14.399 | 18.187 | 1.00 | 10.46 |
| ATOM | 6827 | CB | LYS | 1012 | −14.130 | −15.928 | 18.292 | 1.00 | 12.52 |
| ATOM | 6828 | CG | LYS | 1012 | −15.317 | −16.422 | 19.124 | 1.00 | 14.74 |
| ATOM | 6829 | CD | LYS | 1012 | −15.735 | −17.857 | 18.795 | 1.00 | 15.78 |
| ATOM | 6830 | CE | LYS | 1012 | −14.687 | −18.869 | 19.223 | 1.00 | 14.61 |
| ATOM | 6831 | NZ | LYS | 1012 | −14.548 | −18.899 | 20.722 | 1.00 | 13.04 |
| ATOM | 6832 | C | LYS | 1012 | −14.050 | −13.810 | 19.590 | 1.00 | 11.96 |
| ATOM | 6833 | O | LYS | 1012 | −13.015 | −13.831 | 20.261 | 1.00 | 13.92 |
| ATOM | 6834 | N | ILE | 1013 | −15.186 | −13.280 | 20.028 | 1.00 | 12.31 |
| ATOM | 6835 | CA | ILE | 1013 | −15.292 | −12.693 | 21.357 | 1.00 | 13.37 |
| ATOM | 6836 | CB | ILE | 1013 | −15.325 | −11.149 | 21.278 | 1.00 | 13.64 |
| ATOM | 6837 | CG2 | ILE | 1013 | −13.959 | −10.625 | 20.802 | 1.00 | 13.46 |
| ATOM | 6838 | CG1 | ILE | 1013 | −16.419 | −10.690 | 20.313 | 1.00 | 14.17 |
| ATOM | 6839 | CD1 | ILE | 1013 | −16.605 | −9.181 | 20.299 | 1.00 | 16.29 |
| ATOM | 6840 | C | ILE | 1013 | −16.540 | −13.213 | 22.062 | 1.00 | 14.88 |
| ATOM | 6841 | O | ILE | 1013 | −17.538 | −13.525 | 21.414 | 1.00 | 14.92 |
| ATOM | 6842 | N | GLU | 1014 | −16.475 | −13.304 | 23.388 | 1.00 | 14.84 |
| ATOM | 6843 | CA | GLU | 1014 | −17.581 | −13.814 | 24.199 | 1.00 | 17.49 |
| ATOM | 6844 | CB | GLU | 1014 | −17.039 | −14.690 | 25.334 | 1.00 | 14.94 |
| ATOM | 6845 | CG | GLU | 1014 | −16.318 | −15.943 | 24.884 | 1.00 | 18.47 |
| ATOM | 6846 | CD | GLU | 1014 | −15.753 | −16.757 | 26.052 | 1.00 | 18.83 |
| ATOM | 6847 | OE1 | GLU | 1014 | −16.166 | −16.532 | 27.205 | 1.00 | 20.52 |
| ATOM | 6848 | OE2 | GLU | 1014 | −14.900 | −17.629 | 25.811 | 1.00 | 20.24 |
| ATOM | 6849 | C | GLU | 1014 | −18.441 | −12.722 | 24.818 | 1.00 | 17.91 |
| ATOM | 6850 | O | GLU | 1014 | −17.928 | −11.775 | 25.410 | 1.00 | 18.79 |
| ATOM | 6851 | N | GLY | 1015 | −19.755 | −12.862 | 24.704 | 1.00 | 19.87 |
| ATOM | 6852 | CA | GLY | 1015 | −20.623 | −11.864 | 25.292 | 1.00 | 20.15 |
| ATOM | 6853 | C | GLY | 1015 | −21.824 | −11.530 | 24.445 | 1.00 | 22.90 |
| ATOM | 6854 | O | GLY | 1015 | −21.869 | −11.843 | 23.254 | 1.00 | 22.51 |
| ATOM | 6855 | N | GLY | 1016 | −22.802 | −10.879 | 25.064 | 1.00 | 23.73 |
| ATOM | 6856 | CA | GLY | 1016 | −24.010 | −10.523 | 24.345 | 1.00 | 24.26 |
| ATOM | 6857 | C | GLY | 1016 | −24.190 | −9.048 | 24.052 | 1.00 | 24.54 |
| ATOM | 6858 | O | GLY | 1016 | −23.329 | −8.408 | 23.448 | 1.00 | 23.49 |
| ATOM | 6859 | N | GLU | 1017 | −25.329 | −8.518 | 24.486 | 1.00 | 24.54 |
| ATOM | 6860 | CA | GLU | 1017 | −25.685 | −7.118 | 24.273 | 1.00 | 25.84 |
| ATOM | 6861 | CB | GLU | 1017 | −26.866 | −6.730 | 25.170 | 1.00 | 29.29 |
| ATOM | 6862 | CG | GLU | 1017 | −28.235 | −6.870 | 24.538 | 1.00 | 37.68 |
| ATOM | 6863 | CD | GLU | 1017 | −28.601 | −5.692 | 23.644 | 1.00 | 39.61 |
| ATOM | 6864 | OE1 | GLU | 1017 | −27.961 | −5.504 | 22.590 | 1.00 | 39.67 |
| ATOM | 6865 | OE2 | GLU | 1017 | −29.533 | −4.943 | 24.012 | 1.00 | 43.46 |
| ATOM | 6866 | C | GLU | 1017 | −24.565 | −6.113 | 24.499 | 1.00 | 22.37 |
| ATOM | 6867 | O | GLU | 1017 | −24.354 | −5.229 | 23.673 | 1.00 | 20.28 |
| ATOM | 6868 | N | TRP | 1018 | −23.857 | −6.240 | 25.617 | 1.00 | 21.77 |
| ATOM | 6869 | CA | TRP | 1018 | −22.794 | −5.295 | 25.939 | 1.00 | 20.32 |
| ATOM | 6870 | CB | TRP | 1018 | −22.173 | −5.621 | 27.309 | 1.00 | 20.17 |
| ATOM | 6871 | CG | TRP | 1018 | −21.268 | −6.820 | 27.342 | 1.00 | 18.75 |
| ATOM | 6872 | CD2 | TRP | 1018 | −19.835 | −6.807 | 27.366 | 1.00 | 18.11 |
| ATOM | 6873 | CE2 | TRP | 1018 | −19.405 | −8.151 | 27.412 | 1.00 | 18.06 |
| ATOM | 6874 | CE3 | TRP | 1018 | −18.872 | −5.790 | 27.350 | 1.00 | 16.22 |
| ATOM | 6875 | CD1 | TRP | 1018 | −21.640 | −8.129 | 27.373 | 1.00 | 20.49 |
| ATOM | 6876 | NE1 | TRP | 1018 | −20.527 | −8.938 | 27.420 | 1.00 | 20.28 |
| ATOM | 6877 | CZ2 | TRP | 1018 | −18.049 | −8.506 | 27.446 | 1.00 | 18.14 |
| ATOM | 6878 | CZ3 | TRP | 1018 | −17.523 | −6.146 | 27.384 | 1.00 | 17.33 |
| ATOM | 6879 | CH2 | TRP | 1018 | −17.129 | −7.495 | 27.430 | 1.00 | 16.12 |
| ATOM | 6880 | C | TRP | 1018 | −21.692 | −5.171 | 24.896 | 1.00 | 18.60 |
| ATOM | 6881 | O | TRP | 1018 | −20.943 | −4.193 | 24.901 | 1.00 | 20.29 |
| ATOM | 6882 | N | LEU | 1019 | −21.612 | −6.143 | 23.992 | 1.00 | 17.89 |
| ATOM | 6883 | CA | LEU | 1019 | −20.591 | −6.150 | 22.942 | 1.00 | 18.29 |
| ATOM | 6884 | CB | LEU | 1019 | −20.134 | −7.581 | 22.671 | 1.00 | 18.15 |
| ATOM | 6885 | CG | LEU | 1019 | −19.257 | −8.224 | 23.742 | 1.00 | 18.92 |
| ATOM | 6886 | CD1 | LEU | 1019 | −18.970 | −9.657 | 23.341 | 1.00 | 18.85 |
| ATOM | 6887 | CD2 | LEU | 1019 | −17.969 | −7.431 | 23.886 | 1.00 | 17.98 |
| ATOM | 6888 | C | LEU | 1019 | −21.024 | −5.538 | 21.613 | 1.00 | 19.71 |
| ATOM | 6889 | O | LEU | 1019 | −20.206 | −5.395 | 20.707 | 1.00 | 18.84 |
| ATOM | 6890 | N | VAL | 1020 | −22.301 | −5.190 | 21.496 | 1.00 | 19.00 |
| ATOM | 6891 | CA | VAL | 1020 | −22.833 | −4.612 | 20.272 | 1.00 | 19.17 |
| ATOM | 6892 | CB | VAL | 1020 | −24.281 | −4.105 | 20.489 | 1.00 | 20.01 |
| ATOM | 6893 | CG1 | VAL | 1020 | −24.714 | −3.204 | 19.347 | 1.00 | 18.49 |
| ATOM | 6894 | CG2 | VAL | 1020 | −25.224 | −5.301 | 20.579 | 1.00 | 19.83 |
| ATOM | 6895 | C | VAL | 1020 | −21.987 | −3.478 | 19.708 | 1.00 | 20.01 |
| ATOM | 6896 | O | VAL | 1020 | −21.596 | −3.505 | 18.540 | 1.00 | 20.16 |
| ATOM | 6897 | N | GLU | 1021 | −21.697 | −2.486 | 20.539 | 1.00 | 20.60 |
| ATOM | 6898 | CA | GLU | 1021 | −20.899 | −1.351 | 20.107 | 1.00 | 21.01 |
| ATOM | 6899 | CB | GLU | 1021 | −20.744 | −0.355 | 21.254 | 1.00 | 26.20 |
| ATOM | 6900 | CG | GLU | 1021 | −19.763 | 0.761 | 20.952 | 1.00 | 30.81 |
| ATOM | 6901 | CD | GLU | 1021 | −19.791 | 1.857 | 21.994 | 1.00 | 35.68 |
| ATOM | 6902 | OE1 | GLU | 1021 | −19.602 | 1.551 | 23.193 | 1.00 | 38.35 |
| ATOM | 6903 | OE2 | GLU | 1021 | −20.002 | 3.026 | 21.606 | 1.00 | 38.75 |
| ATOM | 6904 | C | GLU | 1021 | −19.523 | −1.772 | 19.606 | 1.00 | 19.64 |
| ATOM | 6905 | O | GLU | 1021 | −19.062 | −1.327 | 18.555 | 1.00 | 18.56 |
| ATOM | 6906 | N | THR | 1022 | −18.862 | −2.624 | 20.375 | 1.00 | 18.69 |
| ATOM | 6907 | CA | THR | 1022 | −17.539 | −3.107 | 20.010 | 1.00 | 17.98 |
| ATOM | 6909 | CB | THR | 1022 | −17.001 | −4.052 | 21.103 | 1.00 | 18.81 |
| ATOM | 6909 | OG1 | THR | 1022 | −16.885 | −3.323 | 22.329 | 1.00 | 16.49 |
| ATOM | 6910 | CG2 | THR | 1022 | −15.633 | −4.606 | 20.721 | 1.00 | 17.33 |
| ATOM | 6911 | C | THR | 1022 | −17.575 | −3.831 | 18.664 | 1.00 | 17.26 |
| ATOM | 6912 | O | THR | 1022 | −16.707 | −3.630 | 17.808 | 1.00 | 18.36 |
| ATOM | 6913 | N | VAL | 1023 | −18.588 | −4.670 | 18.478 | 1.00 | 17.37 |
| ATOM | 6914 | CA | VAL | 1023 | −18.742 | −5.415 | 17.232 | 1.00 | 16.94 |
| ATOM | 6915 | CB | VAL | 1023 | −19.918 | −6.402 | 17.317 | 1.00 | 16.32 |
| ATOM | 6916 | CG1 | VAL | 1023 | −20.170 | −7.033 | 15.937 | 1.00 | 19.55 |
| ATOM | 6917 | CG2 | VAL | 1023 | −19.606 | −7.484 | 18.338 | 1.00 | 17.66 |
| ATOM | 6918 | C | VAL | 1023 | −18.972 | −4.484 | 16.048 | 1.00 | 18.30 |
| ATOM | 6919 | O | VAL | 1023 | −18.386 | −4.666 | 14.976 | 1.00 | 16.65 |
| ATOM | 6920 | N | GLN | 1024 | −19.828 | −3.488 | 16.242 | 1.00 | 18.44 |
| ATOM | 6921 | CA | GLN | 1024 | −20.125 | −2.540 | 15.169 | 1.00 | 20.28 |
| ATOM | 6922 | CB | GLN | 1024 | −21.152 | −1.498 | 15.631 | 1.00 | 22.35 |
| ATOM | 6923 | CG | GLN | 1024 | −22.494 | −2.088 | 16.057 | 1.00 | 28.92 |
| ATOM | 6924 | CD | GLN | 1024 | −23.512 | −1.029 | 16.479 | 1.00 | 31.59 |
| ATOM | 6925 | OE1 | GLN | 1024 | −23.238 | −0.195 | 17.349 | 1.00 | 32.26 |
| ATOM | 6926 | NE2 | GLN | 1024 | −24.700 | −1.069 | 15.871 | 1.00 | 31.37 |
| ATOM | 6927 | C | GLN | 1024 | −18.841 | −1.840 | 14.740 | 1.00 | 18.28 |
| ATOM | 6928 | O | GLN | 1024 | −18.519 | −1.791 | 13.554 | 1.00 | 19.29 |
| ATOM | 6929 | N | MET | 1025 | −18.104 | −1.319 | 15.717 | 1.00 | 16.21 |
| ATOM | 6930 | CA | MET | 1025 | −16.866 | −0.608 | 15.445 | 1.00 | 16.11 |
| ATOM | 6931 | CB | MET | 1025 | −16.388 | 0.105 | 16.716 | 1.00 | 17.05 |
| ATOM | 6932 | CG | MET | 1025 | −17.392 | 1.124 | 17.243 | 1.00 | 19.13 |
| ATOM | 6933 | SD | MET | 1025 | −16.834 | 1.990 | 18.708 | 1.00 | 22.52 |
| ATOM | 6934 | CE | MET | 1025 | −15.842 | 3.246 | 17.969 | 1.00 | 20.06 |
| ATOM | 6935 | C | MET | 1025 | −15.761 | −1.493 | 14.871 | 1.00 | 15.89 |
| ATOM | 6936 | O | MET | 1025 | −15.026 | −1.070 | 13.978 | 1.00 | 15.39 |
| ATOM | 6937 | N | LEU | 1026 | −15.630 | −2.719 | 15.374 | 1.00 | 15.63 |
| ATOM | 6938 | CA | LEU | 1026 | −14.602 | −3.614 | 14.852 | 1.00 | 14.77 |
| ATOM | 6939 | CB | LEU | 1026 | −14.660 | −4.970 | 15.563 | 1.00 | 13.09 |
| ATOM | 6940 | CG | LEU | 1026 | −13.841 | −4.992 | 16.852 | 1.00 | 12.23 |
| ATOM | 6941 | CD1 | LEU | 1026 | −14.160 | −6.247 | 17.654 | 1.00 | 10.28 |
| ATOM | 6942 | CD2 | LEU | 1026 | −12.354 | −4.920 | 16.493 | 1.00 | 9.62 |
| ATOM | 6943 | C | LEU | 1026 | −14.771 | −3.821 | 13.353 | 1.00 | 15.22 |
| ATOM | 6944 | O | LEU | 1026 | −13.810 | −3.709 | 12.588 | 1.00 | 13.79 |
| ATOM | 6945 | N | THR | 1027 | −16.003 | −4.115 | 12.947 | 1.00 | 18.88 |
| ATOM | 6946 | CA | THR | 1027 | −16.337 | −4.344 | 11.544 | 1.00 | 23.03 |
| ATOM | 6947 | CB | THR | 1027 | −17.863 | −4.600 | 11.369 | 1.00 | 25.20 |
| ATOM | 6948 | OG1 | THR | 1027 | −18.251 | −5.736 | 12.150 | 1.00 | 29.61 |
| ATOM | 6949 | CG2 | THR | 1027 | −18.198 | −4.884 | 9.905 | 1.00 | 30.36 |
| ATOM | 6950 | C | THR | 1027 | −15.923 | −3.177 | 10.645 | 1.00 | 23.34 |
| ATOM | 6951 | O | THR | 1027 | −15.251 | −3.378 | 9.630 | 1.00 | 21.92 |
| ATOM | 6952 | N | GLU | 1028 | −16.315 | −1.957 | 11.005 | 1.00 | 23.53 |
| ATOM | 6953 | CA | GLU | 1028 | −15.945 | −0.807 | 10.181 | 1.00 | 24.72 |
| ATOM | 6954 | CB | GLU | 1028 | −16.678 | 0.466 | 10.643 | 1.00 | 27.14 |
| ATOM | 6955 | CG | GLU | 1028 | −17.060 | 0.487 | 12.105 | 1.00 | 28.12 |
| ATOM | 6956 | CD | GLU | 1028 | −17.832 | 1.740 | 12.511 | 1.00 | 25.37 |
| ATOM | 6957 | OE1 | GLU | 1028 | −18.914 | 2.007 | 11.949 | 1.00 | 26.88 |
| ATOM | 6958 | OE2 | GLU | 1028 | −17.362 | 2.462 | 13.408 | 1.00 | 24.11 |
| ATOM | 6959 | C | GLU | 1028 | −14.433 | −0.597 | 10.173 | 1.00 | 24.26 |
| ATOM | 6960 | O | GLU | 1028 | −13.895 | 0.084 | 9.299 | 1.00 | 24.65 |
| ATOM | 6961 | N | ARG | 1029 | −13.740 | −1.196 | 11.137 | 1.00 | 21.79 |
| ATOM | 6962 | CA | ARG | 1029 | −12.295 | −1.075 | 11.178 | 1.00 | 19.58 |
| ATOM | 6963 | CB | ARG | 1029 | −11.830 | −0.831 | 12.613 | 1.00 | 18.96 |
| ATOM | 6964 | CG | ARG | 1029 | −12.242 | 0.557 | 13.083 | 1.00 | 20.16 |
| ATOM | 6965 | CD | ARG | 1029 | −12.178 | 0.735 | 14.577 | 1.00 | 16.37 |
| ATOM | 6966 | NE | ARG | 1029 | −12.643 | 2.075 | 14.944 | 1.00 | 18.38 |
| ATOM | 6967 | CZ | ARG | 1029 | −13.881 | 2.524 | 14.749 | 1.00 | 15.81 |
| ATOM | 6968 | NH1 | ARG | 1029 | −14.800 | 1.745 | 14.192 | 1.00 | 15.80 |
| ATOM | 6969 | NH2 | ARG | 1029 | −14.202 | 3.762 | 15.104 | 1.00 | 17.73 |
| ATOM | 6970 | C | ARG | 1029 | −11.613 | −2.290 | 10.548 | 1.00 | 18.96 |
| ATOM | 6971 | O | ARG | 1029 | −10.479 | −2.628 | 10.882 | 1.00 | 18.45 |
| ATOM | 6972 | N | ALA | 1030 | −12.341 | −2.939 | 9.638 | 1.00 | 18.34 |
| ATOM | 6973 | CA | ALA | 1030 | −11.849 | −4.081 | 8.869 | 1.00 | 16.44 |
| ATOM | 6974 | CB | ALA | 1030 | −10.532 | −3.702 | 8.187 | 1.00 | 17.45 |
| ATOM | 6975 | C | ALA | 1030 | −11.683 | −5.404 | 9.599 | 1.00 | 15.43 |
| ATOM | 6976 | O | ALA | 1030 | −11.004 | −6.292 | 9.094 | 1.00 | 15.15 |
| ATOM | 6977 | N | VAL | 1031 | −12.305 | −5.544 | 10.766 | 1.00 | 14.41 |
| ATOM | 6978 | CA | VAL | 1031 | −12.205 | −6.783 | 11.529 | 1.00 | 14.99 |
| ATOM | 6979 | CB | VAL | 1031 | −11.741 | −6.513 | 12.990 | 1.00 | 15.55 |
| ATOM | 6980 | CG1 | VAL | 1031 | −11.730 | −7.811 | 13.792 | 1.00 | 15.39 |
| ATOM | 6981 | CG2 | VAL | 1031 | −10.351 | −5.899 | 12.988 | 1.00 | 16.52 |
| ATOM | 6982 | C | VAL | 1031 | −13.523 | −7.548 | 11.587 | 1.00 | 14.09 |
| ATOM | 6983 | O | VAL | 1031 | −14.499 | −7.074 | 12.175 | 1.00 | 14.29 |
| ATOM | 6984 | N | PRO | 1032 | −13.581 | −8.726 | 10.941 | 1.00 | 13.45 |
| ATOM | 6985 | CD | PRO | 1032 | −12.645 | −9.221 | 9.920 | 1.00 | 14.69 |
| ATOM | 6986 | CA | PRO | 1032 | −14.806 | −9.532 | 10.965 | 1.00 | 13.56 |
| ATOM | 6987 | CB | PRO | 1032 | −14.605 | −10.534 | 9.827 | 1.00 | 13.39 |
| ATOM | 6988 | CG | PRO | 1032 | −13.133 | −10.622 | 9.698 | 1.00 | 19.95 |
| ATOM | 6989 | C | PRO | 1032 | −14.872 | −10.187 | 12.337 | 1.00 | 13.75 |
| ATOM | 6990 | O | PRO | 1032 | −13.850 | −10.572 | 12.905 | 1.00 | 14.39 |
| ATOM | 6991 | N | VAL | 1033 | −16.077 | −10.298 | 12.870 | 1.00 | 13.91 |
| ATOM | 6992 | CA | VAL | 1033 | −16.265 | −10.846 | 14.200 | 1.00 | 13.00 |
| ATOM | 6993 | CB | VAL | 1033 | −16.871 | −9.776 | 15.137 | 1.00 | 13.98 |
| ATOM | 6994 | CG1 | VAL | 1033 | −17.112 | −10.343 | 16.534 | 1.00 | 12.93 |
| ATOM | 6995 | CG2 | VAL | 1033 | −15.968 | −8.566 | 15.189 | 1.00 | 12.31 |
| ATOM | 6996 | C | VAL | 1033 | −17.175 | −12.047 | 14.239 | 1.00 | 14.20 |
| ATOM | 6997 | O | VAL | 1033 | −18.219 | −12.078 | 13.580 | 1.00 | 13.95 |
| ATOM | 6998 | N | CYS | 1034 | −16.768 | −13.040 | 15.019 | 1.00 | 12.45 |
| ATOM | 6999 | CA | CYS | 1034 | −17.577 | −14.232 | 15.222 | 1.00 | 14.01 |
| ATOM | 7000 | CB | CYS | 1034 | −16.739 | −15.504 | 15.078 | 1.00 | 12.96 |
| ATOM | 7001 | SG | CYS | 1034 | −17.660 | −17.005 | 15.555 | 1.00 | 15.25 |
| ATOM | 7002 | C | CYS | 1034 | −18.057 | −14.097 | 16.661 | 1.00 | 13.22 |
| ATOM | 7003 | O | CYS | 1034 | −17.251 | −13.898 | 17.569 | 1.00 | 12.90 |
| ATOM | 7004 | N | GLY | 1035 | −19.370 | −14.176 | 16.850 | 1.00 | 13.48 |
| ATOM | 7005 | CA | GLY | 1035 | −19.940 | −14.071 | 18.172 | 1.00 | 15.44 |
| ATOM | 7006 | C | GLY | 1035 | −19.826 | −15.397 | 18.897 | 1.00 | 15.74 |
| ATOM | 7007 | O | GLY | 1035 | −19.491 | −16.422 | 18.294 | 1.00 | 16.26 |
| ATOM | 7008 | N | HIS | 1036 | −20.107 | −15.372 | 20.196 | 1.00 | 15.37 |
| ATOM | 7009 | CA | HIS | 1036 | −20.027 | −16.560 | 21.030 | 1.00 | 15.58 |
| ATOM | 7010 | CB | HIS | 1036 | −18.582 | −16.744 | 21.507 | 1.00 | 16.13 |
| ATOM | 7011 | CG | HIS | 1036 | −18.335 | −18.021 | 22.246 | 1.00 | 17.83 |
| ATOM | 7012 | CD2 | HIS | 1036 | −19.187 | −18.943 | 22.755 | 1.00 | 19.01 |
| ATOM | 7013 | ND1 | HIS | 1036 | −17.065 | −18.479 | 22.525 | 1.00 | 18.18 |
| ATOM | 7014 | CE1 | HIS | 1036 | −17.145 | −19.629 | 23.171 | 1.00 | 20.16 |
| ATOM | 7015 | NE2 | HIS | 1036 | −18.422 | −19.933 | 23.324 | 1.00 | 18.10 |
| ATOM | 7016 | C | HIS | 1036 | −20.971 | −16.397 | 22.223 | 1.00 | 15.56 |
| ATOM | 7017 | O | HIS | 1036 | −20.705 | −15.616 | 23.135 | 1.00 | 14.91 |
| ATOM | 7018 | N | LEU | 1037 | −22.072 | −17.139 | 22.201 | 1.00 | 14.85 |
| ATOM | 7019 | CA | LEU | 1037 | −23.079 | −17.075 | 23.254 | 1.00 | 19.01 |
| ATOM | 7020 | CB | LEU | 1037 | −24.404 | −16.551 | 22.685 | 1.00 | 19.62 |
| ATOM | 7021 | CG | LEU | 1037 | −24.384 | −15.141 | 22.079 | 1.00 | 20.40 |
| ATOM | 7022 | CD1 | LEU | 1037 | −25.681 | −14.870 | 21.323 | 1.00 | 21.01 |
| ATOM | 7023 | CD2 | LEU | 1037 | −24.184 | −14.127 | 23.196 | 1.00 | 22.34 |
| ATOM | 7024 | C | LEU | 1037 | −23.327 | −18.434 | 23.884 | 1.00 | 20.82 |
| ATOM | 7025 | O | LEU | 1037 | −22.970 | −19.475 | 23.320 | 1.00 | 20.02 |
| ATOM | 7026 | N | GLY | 1038 | −23.967 | −18.410 | 25.048 | 1.00 | 22.55 |
| ATOM | 7027 | CA | GLY | 1038 | −24.271 | −19.637 | 25.764 | 1.00 | 25.04 |
| ATOM | 7028 | C | GLY | 1038 | −23.304 | −19.816 | 26.909 | 1.00 | 26.30 |
| ATOM | 7029 | O | GLY | 1038 | −23.171 | −18.944 | 27.767 | 1.00 | 27.66 |
| ATOM | 7030 | N | LEU | 1039 | −22.619 | −20.951 | 26.925 | 1.00 | 28.14 |
| ATOM | 7031 | CA | LEU | 1039 | −21.655 | −21.221 | 27.976 | 1.00 | 29.16 |
| ATOM | 7032 | CB | LEU | 1039 | −21.525 | −22.736 | 28.173 | 1.00 | 31.16 |
| ATOM | 7033 | CG | LEU | 1039 | −20.968 | −23.253 | 29.503 | 1.00 | 33.32 |
| ATOM | 7034 | CD1 | LEU | 1039 | −21.160 | −24.764 | 29.570 | 1.00 | 35.58 |
| ATOM | 7035 | CD2 | LEU | 1039 | −19.503 | −22.891 | 29.641 | 1.00 | 34.73 |
| ATOM | 7036 | C | LEU | 1039 | −20.310 | −20.600 | 27.580 | 1.00 | 28.75 |
| ATOM | 7037 | O | LEU | 1039 | −19.542 | −21.188 | 26.820 | 1.00 | 30.49 |
| ATOM | 7038 | N | THR | 1040 | −20.049 | −19.397 | 28.085 | 1.00 | 29.32 |
| ATOM | 7039 | CA | THR | 1040 | −18.806 | −18.677 | 27.806 | 1.00 | 29.10 |
| ATOM | 7040 | CB | THR | 1040 | −19.043 | −17.147 | 27.758 | 1.00 | 30.20 |
| ATOM | 7041 | OG1 | THR | 1040 | −19.892 | −16.753 | 28.844 | 1.00 | 29.44 |
| ATOM | 7042 | CG2 | THR | 1040 | −19.688 | −16.746 | 26.445 | 1.00 | 32.08 |
| ATOM | 7043 | C | THR | 1040 | −17.781 | −18.999 | 28.893 | 1.00 | 28.48 |
| ATOM | 7044 | O | THR | 1040 | −17.808 | −18.420 | 29.980 | 1.00 | 29.20 |
| ATOM | 7045 | N | PRO | 1041 | −16.850 | −19.920 | 28.597 | 1.00 | 28.41 |
| ATOM | 7046 | CD | PRO | 1041 | −16.620 | −20.464 | 27.246 | 1.00 | 28.58 |
| ATOM | 7047 | CA | PRO | 1041 | −15.797 | −20.363 | 29.518 | 1.00 | 27.50 |
| ATOM | 7048 | CB | PRO | 1041 | −14.949 | −21.298 | 28.652 | 1.00 | 28.87 |
| ATOM | 7049 | CG | PRO | 1041 | −15.152 | −20.773 | 27.279 | 1.00 | 32.35 |
| ATOM | 7050 | C | PRO | 1041 | −14.972 | −19.275 | 30.206 | 1.00 | 26.35 |
| ATOM | 7051 | O | PRO | 1041 | −14.484 | −19.479 | 31.320 | 1.00 | 24.47 |
| ATOM | 7052 | N | GLN | 1042 | −14.802 | −18.126 | 29.558 | 1.00 | 23.87 |
| ATOM | 7053 | CA | GLN | 1042 | −14.043 | −17.050 | 30.188 | 1.00 | 23.26 |
| ATOM | 7054 | CB | GLN | 1042 | −13.872 | −15.868 | 29.223 | 1.00 | 22.94 |
| ATOM | 7055 | CG | GLN | 1042 | −12.682 | −16.011 | 28.273 | 1.00 | 19.12 |
| ATOM | 7056 | CD | GLN | 1042 | −12.640 | −14.915 | 27.228 | 1.00 | 22.27 |
| ATOM | 7057 | OE1 | GLN | 1042 | −13.082 | −13.792 | 27.478 | 1.00 | 21.44 |
| ATOM | 7058 | NE2 | GLN | 1042 | −12.098 | −15.231 | 26.049 | 1.00 | 19.01 |
| ATOM | 7059 | C | GLN | 1042 | −14.737 | −16.597 | 31.475 | 1.00 | 22.74 |
| ATOM | 7060 | O | GLN | 1042 | −14.084 | −16.144 | 32.410 | 1.00 | 22.65 |
| ATOM | 7061 | N | SER | 1043 | −16.059 | −16.739 | 31.532 | 1.00 | 24.41 |
| ATOM | 7062 | CA | SER | 1043 | −16.809 | −16.339 | 32.723 | 1.00 | 24.75 |
| ATOM | 7063 | CB | SER | 1043 | −18.185 | −15.799 | 32.326 | 1.00 | 26.59 |
| ATOM | 7064 | OG | SER | 1043 | −18.069 | −14.611 | 31.563 | 1.00 | 29.05 |
| ATOM | 7065 | C | SER | 1043 | −16.981 | −17.493 | 33.710 | 1.00 | 24.52 |
| ATOM | 7066 | O | SER | 1043 | −17.845 | −17.440 | 34.581 | 1.00 | 25.87 |
| ATOM | 7067 | N | VAL | 1044 | −16.149 | −18.524 | 33.578 | 1.00 | 23.32 |
| ATOM | 7068 | CA | VAL | 1044 | −16.227 | −19.695 | 34.454 | 1.00 | 22.51 |
| ATOM | 7069 | CB | VAL | 1044 | −15.004 | −20.632 | 34.249 | 1.00 | 23.18 |
| ATOM | 7070 | CG1 | VAL | 1044 | −13.718 | −19.911 | 34.614 | 1.00 | 22.95 |
| ATOM | 7071 | CG2 | VAL | 1044 | −15.166 | −21.895 | 35.084 | 1.00 | 22.94 |
| ATOM | 7072 | C | VAL | 1044 | −16.338 | −19.326 | 35.933 | 1.00 | 22.48 |
| ATOM | 7073 | O | VAL | 1044 | −17.060 | −19.982 | 36.682 | 1.00 | 23.24 |
| ATOM | 7074 | N | ASN | 1045 | −15.626 | −18.287 | 36.360 | 1.00 | 22.59 |
| ATOM | 7075 | CA | ASN | 1045 | −15.685 | −17.879 | 37.761 | 1.00 | 24.64 |
| ATOM | 7076 | CB | ASN | 1045 | −14.546 | −16.911 | 38.091 | 1.00 | 23.05 |
| ATOM | 7077 | CG | ASN | 1045 | −13.187 | −17.545 | 37.913 | 1.00 | 21.46 |
| ATOM | 7078 | OD1 | ASN | 1045 | −12.806 | −18.447 | 38.666 | 1.00 | 20.69 |
| ATOM | 7079 | ND2 | ASN | 1045 | −12.453 | −17.096 | 36.904 | 1.00 | 20.14 |
| ATOM | 7080 | C | ASN | 1045 | −17.019 | −17.237 | 38.107 | 1.00 | 25.96 |
| ATOM | 7081 | O | ASN | 1045 | −17.458 | −17.289 | 39.257 | 1.00 | 26.58 |
| ATOM | 7082 | N | ILE | 1046 | −17.666 | −16.633 | 37.115 | 1.00 | 28.57 |
| ATOM | 7083 | CA | ILE | 1046 | −18.962 | −15.997 | 37.342 | 1.00 | 30.78 |
| ATOM | 7084 | CB | ILE | 1046 | −19.397 | −15.116 | 36.141 | 1.00 | 30.35 |
| ATOM | 7085 | CG2 | ILE | 1046 | −20.822 | −14.616 | 36.356 | 1.00 | 29.76 |
| ATOM | 7086 | CG1 | ILE | 1046 | −18.440 | −13.933 | 35.970 | 1.00 | 29.93 |
| ATOM | 7087 | CD1 | ILE | 1046 | −18.419 | −12.976 | 37.145 | 1.00 | 28.61 |
| ATOM | 7088 | C | ILE | 1046 | −20.023 | −17.067 | 37.551 | 1.00 | 32.86 |
| ATOM | 7089 | O | ILE | 1046 | −20.841 | −16.971 | 38.466 | 1.00 | 32.88 |
| ATOM | 7090 | N | PHE | 1047 | −20.008 | −18.084 | 36.693 | 1.00 | 35.65 |
| ATOM | 7091 | CA | PHE | 1047 | −20.977 | −19.174 | 36.783 | 1.00 | 39.34 |
| ATOM | 7092 | CB | PHE | 1047 | −21.125 | −19.878 | 35.430 | 1.00 | 40.96 |
| ATOM | 7093 | CG | PHE | 1047 | −21.373 | −18.946 | 34.277 | 1.00 | 43.16 |
| ATOM | 7094 | CD1 | PHE | 1047 | −22.418 | −18.029 | 34.313 | 1.00 | 45.03 |
| ATOM | 7095 | CD2 | PHE | 1047 | −20.570 | −19.003 | 33.141 | 1.00 | 44.64 |
| ATOM | 7096 | CE1 | PHE | 1047 | −22.660 | −17.177 | 33.234 | 1.00 | 45.71 |
| ATOM | 7097 | CE2 | PHE | 1047 | −20.803 | −18.157 | 32.057 | 1.00 | 45.87 |
| ATOM | 7098 | CZ | PHE | 1047 | −21.851 | −17.243 | 32.104 | 1.00 | 46.15 |
| ATOM | 7099 | C | PHE | 1047 | −20.559 | −20.204 | 37.829 | 1.00 | 40.40 |
| ATOM | 7100 | O | PHE | 1047 | −21.384 | −20.982 | 38.309 | 1.00 | 40.04 |
| ATOM | 7101 | N | GLY | 1048 | −19.275 | −20.208 | 38.174 | 1.00 | 40.74 |
| ATOM | 7102 | CA | GLY | 1048 | −18.780 | −21.157 | 39.153 | 1.00 | 41.82 |
| ATOM | 7103 | C | GLY | 1048 | −18.680 | −22.550 | 38.561 | 1.00 | 43.00 |
| ATOM | 7104 | O | GLY | 1048 | −18.995 | −23.546 | 39.220 | 1.00 | 42.75 |
| ATOM | 7105 | N | GLY | 1049 | −18.262 | −22.617 | 37.306 | 1.00 | 43.62 |
| ATOM | 7106 | CA | GLY | 1049 | −18.140 | −23.893 | 36.629 | 1.00 | 45.01 |
| ATOM | 7107 | C | GLY | 1049 | −18.709 | −23.799 | 35.229 | 1.00 | 46.15 |
| ATOM | 7108 | O | GLY | 1049 | −19.129 | −22.723 | 34.800 | 1.00 | 45.08 |
| ATOM | 7109 | N | TYR | 1050 | −18.725 | −24.919 | 34.513 | 1.00 | 47.77 |
| ATOM | 7110 | CA | TYR | 1050 | −19.255 | −24.940 | 33.156 | 1.00 | 49.51 |
| ATOM | 7111 | CB | TYR | 1050 | −18.312 | −25.707 | 32.227 | 1.00 | 49.59 |
| ATOM | 7112 | CG | TYR | 1050 | −16.897 | −25.181 | 32.246 | 1.00 | 49.48 |
| ATOM | 7113 | CD1 | TYR | 1050 | −15.963 | −25.674 | 33.153 | 1.00 | 49.47 |
| ATOM | 7114 | CE1 | TYR | 1050 | −14.669 | −25.170 | 33.196 | 1.00 | 50.01 |
| ATOM | 7115 | CD2 | TYR | 1050 | −16.499 | −24.166 | 31.376 | 1.00 | 49.76 |
| ATOM | 7116 | CE2 | TYR | 1050 | −15.207 | −23.652 | 31.411 | 1.00 | 49.86 |
| ATOM | 7117 | CZ | TYR | 1050 | −14.298 | −24.159 | 32.323 | 1.00 | 50.33 |
| ATOM | 7118 | OH | TYR | 1050 | −13.019 | −23.657 | 32.370 | 1.00 | 50.67 |
| ATOM | 7119 | C | TYR | 1050 | −20.635 | −25.576 | 33.136 | 1.00 | 50.55 |
| ATOM | 7120 | O | TYR | 1050 | −20.778 | −26.770 | 32.876 | 1.00 | 50.84 |
| ATOM | 7121 | N | LYS | 1051 | −21.648 | −24.760 | 33.412 | 1.00 | 51.54 |
| ATOM | 7122 | CA | LYS | 1051 | −23.031 | −25.218 | 33.444 | 1.00 | 52.63 |
| ATOM | 7123 | CB | LYS | 1051 | −23.738 | −24.620 | 34.662 | 1.00 | 53.67 |
| ATOM | 7124 | CG | LYS | 1051 | −22.963 | −24.790 | 35.960 | 1.00 | 55.80 |
| ATOM | 7125 | CD | LYS | 1051 | −23.561 | −23.961 | 37.087 | 1.00 | 56.82 |
| ATOM | 7126 | CE | LYS | 1051 | −22.680 | −24.006 | 38.328 | 1.00 | 57.73 |
| ATOM | 7127 | NZ | LYS | 1051 | −23.192 | −23.119 | 39.410 | 1.00 | 58.17 |
| ATOM | 7128 | C | LYS | 1051 | −23.756 | −24.804 | 32.165 | 1.00 | 52.42 |
| ATOM | 7129 | O | LYS | 1051 | −23.359 | −23.850 | 31.495 | 1.00 | 51.97 |
| ATOM | 7130 | N | VAL | 1052 | −24.821 | −25.526 | 31.832 | 1.00 | 52.32 |
| ATOM | 7132 | CA | VAL | 1052 | −25.595 | −25.237 | 30.632 | 1.00 | 52.55 |
| ATOM | 7132 | CB | VAL | 1052 | −26.555 | −26.397 | 30.299 | 1.00 | 51.86 |
| ATOM | 7133 | CG1 | VAL | 1052 | −27.330 | −26.087 | 29.025 | 1.00 | 51.77 |
| ATOM | 7134 | CG2 | VAL | 1052 | −25.771 | −27.688 | 30.147 | 1.00 | 51.56 |
| ATOM | 7135 | C | VAL | 1052 | −26.414 | −23.959 | 30.779 | 1.00 | 52.86 |
| ATOM | 7136 | O | VAL | 1052 | −26.992 | −23.698 | 31.834 | 1.00 | 52.72 |
| ATOM | 7137 | N | GLN | 1053 | −26.449 | −23.164 | 29.714 | 1.00 | 53.38 |
| ATOM | 7138 | CA | GLN | 1053 | −27.207 | −21.917 | 29.701 | 1.00 | 53.40 |
| ATOM | 7139 | CB | GLN | 1053 | −26.328 | −20.754 | 29.234 | 1.00 | 53.64 |
| ATOM | 7140 | CG | GLN | 1053 | −25.598 | −20.014 | 30.348 | 1.00 | 54.11 |
| ATOM | 7141 | CD | GLN | 1053 | −24.605 | −20.884 | 31.081 | 1.00 | 53.82 |
| ATOM | 7142 | OE1 | GLN | 1053 | −23.756 | −21.525 | 30.465 | 1.00 | 55.31 |
| ATOM | 7143 | NE2 | GLN | 1053 | −24.697 | −20.904 | 32.406 | 1.00 | 53.40 |
| ATOM | 7144 | C | GLN | 1053 | −28.410 | −22.044 | 28.773 | 1.00 | 53.18 |
| ATOM | 7145 | O | GLN | 2053 | −28.560 | −23.043 | 28.070 | 1.00 | 52.32 |
| ATOM | 7146 | N | GLY | 1054 | −29.261 | −21.024 | 28.773 | 1.00 | 53.68 |
| ATOM | 7147 | CA | GLY | 1054 | −30.440 | −21.045 | 27.927 | 1.00 | 54.63 |
| ATOM | 7148 | C | GLY | 1054 | −31.592 | −21.795 | 28.567 | 1.00 | 55.39 |
| ATOM | 7149 | O | GLY | 1054 | −32.719 | −21.745 | 28.080 | 1.00 | 55.28 |
| ATOM | 7150 | N | ARG | 1055 | −31.303 | −22.495 | 29.660 | 1.00 | 56.01 |
| ATOM | 7151 | CA | ARG | 1055 | −32.314 | −23.255 | 30.382 | 1.00 | 57.40 |
| ATOM | 7152 | CB | ARG | 1055 | −31.674 | −24.003 | 31.556 | 1.00 | 59.12 |
| ATOM | 7153 | CG | ARG | 1055 | −30.714 | −25.123 | 31.266 | 1.00 | 61.02 |
| ATOM | 7154 | CD | ARG | 1055 | −31.455 | −26.328 | 30.604 | 1.00 | 62.44 |
| ATOM | 7155 | NE | ARG | 1055 | −30.566 | −27.459 | 30.347 | 1.00 | 63.71 |
| ATOM | 7156 | CZ | ARG | 1055 | −29.867 | −28.095 | 31.284 | 1.00 | 64.04 |
| ATOM | 7157 | NH1 | ARG | 1055 | −29.949 | −27.713 | 32.551 | 1.00 | 64.28 |
| ATOM | 7158 | NH2 | ARG | 1055 | −29.086 | −29.117 | 30.955 | 1.00 | 64.25 |
| ATOM | 7159 | C | ARG | 1055 | −33.403 | −22.325 | 30.911 | 1.00 | 57.48 |
| ATOM | 7160 | O | ARG | 1055 | −33.223 | −21.667 | 31.936 | 1.00 | 57.81 |
| ATOM | 7161 | N | GLY | 1056 | −34.530 | −22.268 | 30.208 | 1.00 | 57.14 |
| ATOM | 7162 | CA | GLY | 1056 | −35.619 | −21.414 | 30.643 | 1.00 | 56.84 |
| ATOM | 7163 | C | GLY | 1056 | −36.179 | −20.523 | 29.552 | 1.00 | 56.31 |
| ATOM | 7164 | O | GLY | 1056 | −35.678 | −20.507 | 28.427 | 1.00 | 55.89 |
| ATOM | 7165 | N | ASP | 1057 | −37.224 | −19.778 | 29.894 | 1.00 | 56.37 |
| ATOM | 7166 | CA | ASP | 1057 | −37.869 | −18.874 | 28.951 | 1.00 | 56.25 |
| ATOM | 7167 | CB | ASP | 1057 | −39.315 | −18.615 | 29.382 | 1.00 | 57.60 |
| ATOM | 7168 | CG | ASP | 1057 | −40.138 | −19.886 | 29.450 | 1.00 | 58.32 |
| ATOM | 7169 | OD1 | ASP | 1057 | −40.329 | −20.525 | 28.394 | 1.00 | 58.27 |
| ATOM | 7170 | OD2 | ASP | 1057 | −40.589 | −20.248 | 30.560 | 1.00 | 58.42 |
| ATOM | 7171 | C | ASP | 1057 | −37.116 | −17.552 | 28.868 | 1.00 | 55.73 |
| ATOM | 7172 | O | ASP | 1057 | −36.656 | −17.154 | 27.797 | 1.00 | 55.55 |
| ATOM | 7173 | N | GLU | 1058 | −36.990 | −16.878 | 30.005 | 1.00 | 54.61 |
| ATOM | 7174 | CA | GLU | 1058 | −36.295 | −15.600 | 30.059 | 1.00 | 53.66 |
| ATOM | 7175 | CB | GLU | 1058 | −36.187 | −15.125 | 31.510 | 1.00 | 54.74 |
| ATOM | 7176 | CG | GLU | 1058 | −35.451 | −13.809 | 31.680 | 1.00 | 56.54 |
| ATOM | 7177 | CD | GLU | 1058 | −35.511 | −13.289 | 33.103 | 1.00 | 57.81 |
| ATOM | 7178 | OE1 | CLU | 1058 | −36.616 | −12.929 | 33.560 | 1.00 | 58.07 |
| ATOM | 7179 | OE2 | GLU | 1058 | −34.453 | −13.247 | 33.767 | 1.00 | 58.77 |
| ATOM | 7180 | C | GLU | 1058 | −34.906 | −15.700 | 29.436 | 1.00 | 52.45 |
| ATOM | 7181 | O | GLU | 1058 | −34.577 | −14.950 | 28.515 | 1.00 | 52.76 |
| ATOM | 7182 | N | ALA | 1059 | −34.095 | −16.628 | 29.937 | 1.00 | 50.76 |
| ATOM | 7183 | CA | ALA | 1059 | −32.741 | −16.820 | 29.420 | 1.00 | 48.43 |
| ATOM | 7184 | CB | ALA | 1059 | −31.995 | −17.834 | 30.277 | 1.00 | 48.71 |
| ATOM | 7185 | C | ALA | 1059 | −32.790 | −17.290 | 27.969 | 1.00 | 46.48 |
| ATOM | 7186 | O | ALA | 1059 | −31.947 | −16.913 | 27.153 | 1.00 | 46.02 |
| ATOM | 7187 | N | GLY | 1060 | −33.783 | −18.116 | 27.658 | 1.00 | 44.48 |
| ATOM | 7188 | CA | GLY | 1060 | −33.931 | −18.618 | 26.307 | 1.00 | 42.19 |
| ATOM | 7189 | C | GLY | 1060 | −34.145 | −17.491 | 25.315 | 1.00 | 41.58 |
| ATOM | 7190 | O | GLY | 1060 | −33.539 | −17.480 | 24.245 | 1.00 | 41.81 |
| ATOM | 7191 | N | ASP | 1061 | −35.008 | −16.540 | 25.667 | 1.00 | 40.06 |
| ATOM | 7192 | CA | ASP | 1061 | −35.288 | −15.401 | 24.797 | 1.00 | 38.77 |
| ATOM | 7193 | CB | ASP | 1061 | −36.570 | −14.686 | 25.236 | 1.00 | 39.86 |
| ATOM | 7194 | CG | ASP | 1061 | −37.760 | −15.616 | 25.318 | 1.00 | 39.94 |
| ATOM | 7195 | OD1 | ASP | 1061 | −38.037 | −16.323 | 24.328 | 1.00 | 40.73 |
| ATOM | 7196 | OD2 | ASP | 1061 | −38.422 | −15.634 | 26.375 | 1.00 | 41.22 |
| ATOM | 7197 | C | ASP | 1061 | −34.131 | −14.407 | 24.837 | 1.00 | 37.48 |
| ATOM | 7198 | O | ASP | 1061 | −33.857 | −13.719 | 23.854 | 1.00 | 37.88 |
| ATOM | 7199 | N | GLN | 1062 | −33.464 | −14.332 | 25.983 | 1.00 | 35.98 |
| ATOM | 7200 | CA | GLN | 1062 | −32.337 | −13.426 | 26.155 | 1.00 | 34.74 |
| ATOM | 7201 | CB | GLN | 1062 | −31.807 | −13.508 | 27.586 | 1.00 | 35.48 |
| ATOM | 7202 | CG | GLN | 1062 | −30.679 | −12.533 | 27.895 | 1.00 | 36.93 |
| ATOM | 7203 | CD | GLN | 1062 | −31.115 | −11.083 | 27.786 | 1.00 | 38.62 |
| ATOM | 7204 | OE1 | GLN | 1062 | −32.048 | −10.653 | 28.465 | 1.00 | 38.65 |
| ATOM | 7205 | NE2 | GLN | 1062 | −30.439 | −10.320 | 26.933 | 1.00 | 38.92 |
| ATOM | 7206 | C | GLN | 1062 | −31.225 | −13.777 | 25.173 | 1.00 | 33.19 |
| ATOM | 7207 | O | GLN | 1062 | −30.671 | −12.899 | 24.512 | 1.00 | 31.37 |
| ATOM | 7208 | N | LEU | 1063 | −30.904 | −15.064 | 25.082 | 1.00 | 32.49 |
| ATOM | 7209 | CA | LEU | 1063 | −29.861 | −15.531 | 24.176 | 1.00 | 31.76 |
| ATOM | 7210 | CB | LEU | 1063 | −29.584 | −17.025 | 24.405 | 1.00 | 32.31 |
| ATOM | 7211 | CG | LEU | 1063 | −28.900 | −17.413 | 25.721 | 1.00 | 33.38 |
| ATOM | 7212 | CD1 | LEU | 1063 | −28.693 | −18.923 | 25.776 | 1.00 | 34.58 |
| ATOM | 7213 | CD2 | LEU | 1063 | −27.560 | −16.701 | 25.837 | 1.00 | 33.59 |
| ATOM | 7214 | C | LEU | 1063 | −30.243 | −15.287 | 22.718 | 1.00 | 31.49 |
| ATOM | 7215 | O | LEU | 1063 | −29.410 | −14.874 | 21.910 | 1.00 | 29.46 |
| ATOM | 7216 | N | LEU | 1064 | −31.504 | −15.544 | 22.385 | 1.00 | 30.95 |
| ATOM | 7217 | CA | LEU | 1064 | −31.984 | −15.334 | 21.024 | 1.00 | 31.20 |
| ATOM | 7218 | CB | LEU | 1064 | −33.442 | −15.792 | 20.899 | 1.00 | 33.16 |
| ATOM | 7219 | CG | LEU | 1064 | −34.097 | −15.755 | 19.513 | 1.00 | 34.91 |
| ATOM | 7220 | CD1 | LEU | 1064 | −35.286 | −16.709 | 19.482 | 1.00 | 35.64 |
| ATOM | 7221 | CD2 | LEU | 1064 | −34.536 | −14.350 | 19.182 | 1.00 | 34.56 |
| ATOM | 7222 | C | LEU | 1064 | −31.865 | −13.850 | 20.696 | 1.00 | 29.59 |
| ATOM | 7223 | O | LEU | 1064 | −31.476 | −13.474 | 19.589 | 1.00 | 28.93 |
| ATOM | 7224 | N | SER | 1065 | −32.191 | −13.009 | 21.672 | 1.00 | 27.94 |
| ATOM | 7225 | CA | SER | 1065 | −32.106 | −11.572 | 21.475 | 1.00 | 27.91 |
| ATOM | 7226 | CB | SER | 1065 | −32.645 | −10.830 | 22.697 | 1.00 | 28.13 |
| ATOM | 7227 | OG | SER | 1065 | −32.610 | −9.429 | 22.483 | 1.00 | 30.15 |
| ATOM | 7228 | C | SER | 1065 | −30.662 | −11.156 | 21.219 | 1.00 | 26.39 |
| ATOM | 7229 | O | SER | 1065 | −30.389 | −10.386 | 20.300 | 1.00 | 26.00 |
| ATOM | 7230 | N | ASP | 1066 | −29.741 | −11.665 | 22.035 | 1.00 | 25.06 |
| ATOM | 7231 | CA | ASP | 1066 | −28.320 | −11.340 | 21.880 | 1.00 | 24.78 |
| ATOM | 7232 | CB | ASP | 1066 | −27.491 | −11.989 | 22.989 | 1.00 | 25.95 |
| ATOM | 7233 | CG | ASP | 1066 | −27.730 | −11.348 | 24.338 | 1.00 | 28.70 |
| ATOM | 7234 | OD1 | ASP | 1066 | −27.860 | −10.107 | 24.385 | 1.00 | 30.67 |
| ATOM | 7235 | OD2 | ASP | 1066 | −27.775 | −12.077 | 25.351 | 1.00 | 31.26 |
| ATOM | 7236 | C | ASP | 1066 | −27.783 | −11.782 | 20.524 | 1.00 | 23.52 |
| ATOM | 7237 | O | ASP | 1066 | −26.968 | −11.089 | 19.915 | 1.00 | 23.49 |
| ATOM | 7238 | N | ALA | 1067 | −28.235 | −12.945 | 20.062 | 1.00 | 23.51 |
| ATOM | 7239 | CA | ALA | 1067 | −27.813 | −13.478 | 18.776 | 1.00 | 22.29 |
| ATOM | 7240 | CB | ALA | 1067 | −28.450 | −14.842 | 18.547 | 1.00 | 22.43 |
| ATOM | 7241 | C | ALA | 1067 | −28.216 | −12.516 | 17.665 | 1.00 | 22.46 |
| ATOM | 7242 | O | ALA | 1067 | −27.410 | −12.164 | 16.804 | 1.00 | 20.38 |
| ATOM | 7243 | N | LEU | 1068 | −29.472 | −12.091 | 17.683 | 1.00 | 23.01 |
| ATOM | 7244 | CA | LEU | 1068 | −29.961 | −11.167 | 16.668 | 1.00 | 22.75 |
| ATOM | 7245 | CB | LEU | 1068 | −31.464 | −10.947 | 16.844 | 1.00 | 22.25 |
| ATOM | 7246 | CG | LEU | 1068 | −32.364 | −12.126 | 16.480 | 1.00 | 23.59 |
| ATOM | 7247 | CD1 | LEU | 1068 | −33.765 | −11.876 | 17.028 | 1.00 | 25.32 |
| ATOM | 7248 | CD2 | LEU | 1068 | −32.399 | −12.310 | 14.959 | 1.00 | 24.63 |
| ATOM | 7249 | C | LEU | 1068 | −29.224 | −9.838 | 16.784 | 1.00 | 20.46 |
| ATOM | 7250 | O | LEU | 1068 | −28.926 | −9.210 | 15.781 | 1.00 | 20.46 |
| ATOM | 7251 | N | ALA | 1069 | −28.928 | −9.436 | 18.016 | 1.00 | 20.31 |
| ATOM | 7252 | CA | ALA | 1069 | −28.234 | −8.186 | 18.284 | 1.00 | 20.51 |
| ATOM | 7253 | CB | ALA | 1069 | −28.237 | −7.899 | 19.783 | 1.00 | 21.24 |
| ATOM | 7254 | C | ALA | 1069 | −26.803 | −8.201 | 17.757 | 1.00 | 18.78 |
| ATOM | 7255 | O | ALA | 1069 | −26.326 | −7.211 | 17.203 | 1.00 | 17.76 |
| ATOM | 7256 | N | LEU | 1070 | −26.117 | −9.325 | 17.929 | 1.00 | 19.42 |
| ATOM | 7257 | CA | LEU | 1070 | −24.745 | −9.440 | 17.455 | 1.00 | 18.83 |
| ATOM | 7258 | CB | LEU | 1070 | −24.092 | −10.719 | 18.005 | 1.00 | 18.63 |
| ATOM | 7259 | CG | LEU | 1070 | −23.813 | −10.721 | 19.516 | 1.00 | 17.66 |
| ATOM | 7260 | CD1 | LEU | 1070 | −23.361 | −12.111 | 19.965 | 1.00 | 17.08 |
| ATOM | 7261 | CD2 | LEU | 1070 | −22.750 | −9.689 | 19.849 | 1.00 | 16.40 |
| ATOM | 7262 | C | LEU | 1070 | −24.741 | −9.435 | 15.930 | 1.00 | 18.26 |
| ATOM | 7263 | O | LEU | 1070 | −23.897 | −8.791 | 15.308 | 1.00 | 16.25 |
| ATOM | 7264 | N | GLU | 1071 | −25.692 | −10.141 | 15.327 | 1.00 | 19.53 |
| ATOM | 7265 | CA | GLU | 1071 | −25.789 | −10.178 | 13.872 | 1.00 | 19.82 |
| ATOM | 7266 | CB | GLU | 1071 | −26.907 | −11.137 | 13.445 | 1.00 | 22.79 |
| ATOM | 7267 | CG | GLU | 1071 | −27.158 | −11.196 | 11.944 | 1.00 | 23.57 |
| ATOM | 7268 | CD | GLU | 1071 | −28.293 | −12.133 | 11.587 | 1.00 | 25.87 |
| ATOM | 7269 | OE1 | GLU | 1071 | −29.412 | −11.938 | 12.111 | 1.00 | 26.11 |
| ATOM | 7270 | OE2 | GLU | 1071 | −28.075 | −13.063 | 10.783 | 1.00 | 27.62 |
| ATOM | 7271 | C | GLU | 1071 | −26.069 | −8.775 | 13.324 | 1.00 | 19.77 |
| ATOM | 7272 | O | GLU | 1071 | −25.424 | −8.339 | 12.374 | 1.00 | 17.58 |
| ATOM | 7273 | N | ALA | 1072 | −27.028 | −8.071 | 13.922 | 1.00 | 20.66 |
| ATOM | 7274 | CA | ALA | 1072 | −27.368 | −6.721 | 13.467 | 1.00 | 20.74 |
| ATOM | 7275 | CB | ALA | 1072 | −28.569 | −6.182 | 14.245 | 1.00 | 21.28 |
| ATOM | 7276 | C | ALA | 1072 | −26.176 | −5.786 | 13.630 | 1.00 | 22.41 |
| ATOM | 7277 | O | ALA | 1072 | −26.034 | −4.804 | 12.898 | 1.00 | 23.66 |
| ATOM | 7278 | N | ALA | 1073 | −25.319 | −6.099 | 14.598 | 1.00 | 22.57 |
| ATOM | 7279 | CA | ALA | 1073 | −24.130 | −5.295 | 14.856 | 1.00 | 19.20 |
| ATOM | 7280 | CB | ALA | 1073 | −23.572 | −5.616 | 16.241 | 1.00 | 20.25 |
| ATOM | 7281 | C | ALA | 1073 | −23.058 | −5.530 | 13.792 | 1.00 | 19.63 |
| ATOM | 7282 | O | ALA | 1073 | −22.136 | −4.730 | 13.647 | 1.00 | 19.20 |
| ATOM | 7283 | N | GLY | 1074 | −23.168 | −6.628 | 13.049 | 1.00 | 17.74 |
| ATOM | 7284 | CA | GLY | 1074 | −22.175 | −6.890 | 12.018 | 1.00 | 17.83 |
| ATOM | 7285 | C | GLY | 1074 | −21.454 | −8.225 | 12.112 | 1.00 | 17.24 |
| ATOM | 7286 | O | GLY | 1074 | −20.632 | −8.545 | 11.250 | 1.00 | 17.98 |
| ATOM | 7287 | N | ALA | 1075 | −21.739 | −9.005 | 13.149 | 1.00 | 17.76 |
| ATOM | 7288 | CA | ALA | 1075 | −21.096 | −10.310 | 13.286 | 1.00 | 17.79 |
| ATOM | 7289 | CB | ALA | 1075 | −21.544 | −10.994 | 14.581 | 1.00 | 16.20 |
| ATOM | 7290 | C | ALA | 1075 | −21.479 | −11.160 | 12.080 | 1.00 | 18.93 |
| ATOM | 7291 | O | ALA | 1075 | −22.659 | −11.237 | 11.709 | 1.00 | 18.99 |
| ATOM | 7292 | N | GLN | 1076 | −20.478 | −11.790 | 11.468 | 1.00 | 17.65 |
| ATOM | 7293 | CA | GLN | 1076 | −20.685 | −12.625 | 10.283 | 1.00 | 19.07 |
| ATOM | 7294 | CB | GLN | 1076 | −19.575 | −12.363 | 9.258 | 1.00 | 20.10 |
| ATOM | 7295 | CG | GLN | 1076 | −19.644 | −10.967 | 8.666 | 1.00 | 21.35 |
| ATOM | 7296 | CD | GLN | 1076 | −18.519 | −10.664 | 7.700 | 1.00 | 23.80 |
| ATOM | 7297 | OE1 | GLN | 1076 | −18.528 | −11.110 | 6.547 | 1.00 | 28.97 |
| ATOM | 7298 | NE2 | GLN | 1076 | −17.540 | −9.900 | 8.164 | 1.00 | 19.71 |
| ATOM | 7299 | C | GLN | 1076 | −20.757 | −14.110 | 10.604 | 1.00 | 18.87 |
| ATOM | 7300 | O | GLN | 1076 | −21.044 | −14.933 | 9.731 | 1.00 | 21.05 |
| ATOM | 7301 | N | LEU | 1077 | −20.505 | −14.435 | 11.867 | 1.00 | 19.04 |
| ATOM | 7302 | CA | LEU | 1077 | −20.540 | −15.804 | 12.361 | 1.00 | 18.01 |
| ATOM | 7303 | CB | LEU | 1077 | −19.163 | −16.467 | 12.210 | 1.00 | 21.10 |
| ATOM | 7304 | CG | LEU | 1077 | −18.902 | −17.310 | 10.966 | 1.00 | 22.57 |
| ATOM | 7305 | CD1 | LEU | 1077 | −17.468 | −17.821 | 10.996 | 1.00 | 24.38 |
| ATOM | 7306 | CD2 | LEU | 1077 | −19.878 | −18.477 | 10.920 | 1.00 | 23.84 |
| ATOM | 7307 | C | LEU | 1077 | −20.925 | −15.799 | 13.831 | 1.00 | 17.60 |
| ATOM | 7308 | O | LEU | 1077 | −20.699 | −14.816 | 14.536 | 1.00 | 17.24 |
| ATOM | 7309 | N | LEU | 1078 | −21.514 | −16.895 | 14.293 | 1.00 | 16.91 |
| ATOM | 7310 | CA | LEU | 1078 | −21.898 | −17.013 | 15.700 | 1.00 | 17.04 |
| ATOM | 7311 | CB | LEU | 1078 | −23.371 | −16.631 | 15.904 | 1.00 | 18.53 |
| ATOM | 7312 | CG | LEU | 1078 | −23.888 | −16.847 | 17.336 | 1.00 | 19.84 |
| ATOM | 7313 | CD1 | LEU | 1078 | −23.257 | −15.825 | 18.288 | 1.00 | 19.36 |
| ATOM | 7314 | CD2 | LEU | 1078 | −25.401 | −16.719 | 17.362 | 1.00 | 20.91 |
| ATOM | 7315 | C | LEU | 1078 | −21.695 | −18.434 | 16.206 | 1.00 | 17.33 |
| ATOM | 7316 | O | LEU | 1078 | −22.124 | −19.395 | 15.563 | 1.00 | 18.22 |
| ATOM | 7317 | N | VAL | 1079 | −21.037 | −18.555 | 17.357 | 1.00 | 16.40 |
| ATOM | 7318 | CA | VAL | 1079 | −20.806 | −19.847 | 17.999 | 1.00 | 15.90 |
| ATOM | 7319 | CB | VAL | 1079 | −19.364 | −19.972 | 18.562 | 1.00 | 16.02 |
| ATOM | 7320 | CG1 | VAL | 1079 | −19.271 | −21.169 | 19.519 | 1.00 | 15.56 |
| ATOM | 7321 | CG2 | VAL | 1079 | −18.376 | −20.161 | 17.418 | 1.00 | 11.83 |
| ATOM | 7322 | C | VAL | 1079 | −21.780 | −19.977 | 19.163 | 1.00 | 18.15 |
| ATOM | 7323 | O | VAL | 1079 | −21.915 | −19.056 | 19.971 | 1.00 | 20.36 |
| ATOM | 7324 | N | LEU | 1080 | −22.467 | −21.113 | 19.225 | 1.00 | 20.11 |
| ATOM | 7325 | CA | LEU | 1080 | −23.420 | −21.401 | 20.296 | 1.00 | 20.37 |
| ATOM | 7326 | CB | LEU | 1080 | −24.792 | −21.775 | 19.730 | 1.00 | 21.80 |
| ATOM | 7327 | CG | LEU | 1080 | −25.778 | −20.659 | 19.401 | 1.00 | 24.35 |
| ATOM | 7328 | CD1 | LEU | 1080 | −27.092 | −21.278 | 18.936 | 1.00 | 22.66 |
| ATOM | 7329 | CD2 | LEU | 1080 | −26.010 | −19.794 | 20.637 | 1.00 | 23.99 |
| ATOM | 7330 | C | LEU | 1080 | −22.876 | −22.584 | 21.085 | 1.00 | 19.26 |
| ATOM | 7331 | O | LEU | 1080 | −22.673 | −23.659 | 20.526 | 1.00 | 18.71 |
| ATOM | 7332 | N | GLU | 1081 | −22.656 | −22.387 | 22.379 | 1.00 | 20.64 |
| ATOM | 7333 | CA | GLU | 1081 | −22.110 | −23.439 | 23.224 | 1.00 | 21.53 |
| ATOM | 7334 | CB | GLU | 1081 | −20.760 | −22.988 | 23.790 | 1.00 | 21.79 |
| ATOM | 7335 | CG | GLU | 1081 | −20.131 | −23.952 | 24.779 | 1.00 | 21.93 |
| ATOM | 7336 | CD | GLU | 1081 | −18.676 | −23.627 | 25.073 | 1.00 | 25.66 |
| ATOM | 7337 | OE1 | GLU | 1081 | −18.223 | −22.519 | 24.712 | 1.00 | 24.72 |
| ATOM | 7338 | OE2 | GLU | 1081 | −17.989 | −24.481 | 25.673 | 1.00 | 25.08 |
| ATOM | 7339 | C | GLU | 1081 | −23.018 | −23.897 | 24.364 | 1.00 | 22.06 |
| ATOM | 7340 | O | GLU | 1081 | −23.556 | −23.083 | 25.118 | 1.00 | 21.49 |
| ATOM | 7341 | N | CYS | 1082 | −23.166 | −25.215 | 24.471 | 1.00 | 23.09 |
| ATOM | 7342 | CA | CYS | 1082 | −23.970 | −25.861 | 25.503 | 1.00 | 23.95 |
| ATOM | 7343 | CB | CYS | 1082 | −23.146 | −26.008 | 26.776 | 1.00 | 25.09 |
| ATOM | 7344 | SG | CYS | 1082 | −21.655 | −26.980 | 26.505 | 1.00 | 27.14 |
| ATOM | 7345 | C | CYS | 1082 | −25.276 | −25.148 | 25.800 | 1.00 | 25.48 |
| ATOM | 7346 | O | CYS | 1082 | −25.409 | −24.431 | 26.795 | 1.00 | 25.29 |
| ATOM | 7347 | N | VAL | 1083 | −26.238 | −25.372 | 24.915 | 1.00 | 26.92 |
| ATOM | 7348 | CA | VAL | 1083 | −27.561 | −24.784 | 25.012 | 1.00 | 29.17 |
| ATOM | 7349 | CB | VAL | 1083 | −27.646 | −23.516 | 24.129 | 1.00 | 28.96 |
| ATOM | 7350 | CG1 | VAL | 1083 | −27.611 | −23.900 | 22.655 | 1.00 | 30.28 |
| ATOM | 7351 | CG2 | VAL | 1083 | −28.898 | −22.729 | 24.450 | 1.00 | 32.97 |
| ATOM | 7352 | C | VAL | 1083 | −28.559 | −25.830 | 24.508 | 1.00 | 29.38 |
| ATOM | 7353 | O | VAL | 1083 | −28.276 | −26.570 | 23.559 | 1.00 | 28.35 |
| ATOM | 7354 | N | PRO | 1084 | −29.740 | −25.911 | 25.138 | 1.00 | 30.34 |
| ATOM | 7355 | CD | PRO | 1084 | −30.314 | −25.021 | 26.160 | 1.00 | 29.84 |
| ATOM | 7356 | CA | PRO | 1084 | −30.728 | −26.896 | 24.688 | 1.00 | 30.70 |
| ATOM | 7357 | CB | PRO | 1084 | −31.989 | −26.510 | 25.471 | 1.00 | 30.48 |
| ATOM | 7358 | CG | PRO | 1084 | −31.775 | −25.059 | 25.807 | 1.00 | 31.98 |
| ATOM | 7359 | C | PRO | 1084 | −30.928 | −26.865 | 23.176 | 1.00 | 31.50 |
| ATOM | 7360 | O | PRO | 1084 | −31.018 | −25.794 | 22.572 | 1.00 | 32.64 |
| ATOM | 7361 | N | VAL | 1085 | −30.983 | −28.049 | 22.574 | 1.00 | 32.04 |
| ATOM | 7362 | CA | VAL | 1085 | −31.157 | −28.185 | 21.134 | 1.00 | 32.80 |
| ATOM | 7363 | CB | VAL | 1085 | −31.465 | −29.648 | 20.748 | 1.00 | 33.17 |
| ATOM | 7364 | CG1 | VAL | 1085 | −31.544 | −29.780 | 19.239 | 1.00 | 32.51 |
| ATOM | 7365 | CG2 | VAL | 1085 | −30.392 | −30.573 | 21.309 | 1.00 | 32.67 |
| ATOM | 7366 | C | VAL | 1085 | −32.291 | −27.305 | 20.632 | 1.00 | 34.32 |
| ATOM | 7367 | O | VAL | 1085 | −32.224 | −26.755 | 19.530 | 1.00 | 33.11 |
| ATOM | 7368 | N | GLU | 1086 | −33.325 | −27.176 | 21.457 | 1.00 | 36.11 |
| ATOM | 7369 | CA | GLU | 1086 | −34.500 | −26.371 | 21.131 | 1.00 | 38.46 |
| ATOM | 7370 | CB | GLU | 1086 | −35.479 | −26.364 | 22.313 | 1.00 | 40.09 |
| ATOM | 7371 | CG | GLU | 1086 | −35.428 | −27.605 | 23.195 | 1.00 | 42.70 |
| ATOM | 7372 | CD | GLU | 1086 | −35.496 | −28.894 | 22.402 | 1.00 | 44.30 |
| ATOM | 7373 | OE1 | GLU | 1086 | −36.420 | −29.040 | 21.573 | 1.00 | 45.21 |
| ATOM | 7374 | OE2 | GLU | 1086 | −34.626 | −29.764 | 22.614 | 1.00 | 45.27 |
| ATOM | 7375 | C | GLU | 1086 | −34.097 | −24.935 | 20.812 | 1.00 | 37.16 |
| ATOM | 7376 | O | GLU | 1086 | −34.441 | −24.400 | 19.758 | 1.00 | 37.62 |
| ATOM | 7377 | N | LEU | 1087 | −33.370 | −24.316 | 21.736 | 1.00 | 37.02 |
| ATOM | 7378 | CA | LEU | 1087 | −32.928 | −22.941 | 21.562 | 1.00 | 36.84 |
| ATOM | 7379 | CB | LEU | 1087 | −32.192 | −22.455 | 22.813 | 1.00 | 38.04 |
| ATOM | 7380 | CG | LEU | 1087 | −32.583 | −21.085 | 23.384 | 1.00 | 39.52 |
| ATOM | 7381 | CD1 | LEU | 1087 | −31.751 | −20.804 | 24.630 | 1.00 | 40.56 |
| ATOM | 7382 | CD2 | LEU | 1087 | −32.378 | −19.994 | 22.349 | 1.00 | 39.60 |
| ATOM | 7383 | C | LEU | 1087 | −32.020 | −22.817 | 20.345 | 1.00 | 36.08 |
| ATOM | 7384 | O | LEU | 1087 | −32.141 | −21.870 | 19.570 | 1.00 | 35.75 |
| ATOM | 7385 | N | ALA | 1088 | −31.116 | −23.778 | 20.173 | 1.00 | 35.37 |
| ATOM | 7386 | CA | ALA | 1088 | −30.195 | −23.757 | 19.038 | 1.00 | 34.84 |
| ATOM | 7387 | CB | ALA | 1088 | −29.294 | −24.989 | 19.066 | 1.00 | 32.98 |
| ATOM | 7388 | C | ALA | 1088 | −30.965 | −23.700 | 17.722 | 1.00 | 35.07 |
| ATOM | 7389 | O | ALA | 1088 | −30.530 | −23.059 | 16.765 | 1.00 | 35.13 |
| ATOM | 7390 | N | LYS | 1089 | −32.112 | −24.372 | 17.685 | 1.00 | 36.00 |
| ATOM | 7391 | CA | LYS | 1089 | −32.952 | −24.402 | 16.493 | 1.00 | 37.51 |
| ATOM | 7392 | CB | LYS | 1089 | −34.133 | −25.353 | 16.702 | 1.00 | 40.32 |
| ATOM | 7393 | CG | LYS | 1089 | −33.741 | −26.772 | 17.054 | 1.00 | 42.81 |
| ATOM | 7394 | CD | LYS | 1089 | −34.966 | −27.619 | 17.354 | 1.00 | 46.04 |
| ATOM | 7395 | CE | LYS | 1089 | −34.575 | −29.010 | 17.818 | 1.00 | 46.35 |
| ATOM | 7396 | NZ | LYS | 1089 | −35.755 | −29.769 | 18.300 | 1.00 | 47.78 |
| ATOM | 7397 | C | LYS | 1089 | −33.485 | −23.014 | 16.161 | 1.00 | 35.60 |
| ATOM | 7398 | O | LYS | 1089 | −33.330 | −22.536 | 15.039 | 1.00 | 35.12 |
| ATOM | 7399 | N | ARG | 1090 | −34.117 | −22.374 | 17.141 | 1.00 | 35.99 |
| ATOM | 7400 | CA | ARG | 1090 | −34.681 | −21.039 | 16.947 | 1.00 | 36.46 |
| ATOM | 7401 | CB | ARG | 1090 | −35.288 | −20.511 | 18.247 | 1.00 | 38.53 |
| ATOM | 7402 | CG | ARG | 1090 | −36.350 | −21.392 | 18.862 | 1.00 | 42.68 |
| ATOM | 7403 | CD | ARG | 1090 | −37.186 | −20.592 | 19.842 | 1.00 | 45.65 |
| ATOM | 7404 | NE | ARG | 1090 | −36.365 | −19.905 | 20.834 | 1.00 | 49.18 |
| ATOM | 7405 | CZ | ARG | 1090 | −36.842 | −19.046 | 21.730 | 1.00 | 50.27 |
| ATOM | 7406 | NH1 | ARG | 1090 | −38.137 | −18.769 | 21.758 | 1.00 | 51.07 |
| ATOM | 7407 | NH2 | ARG | 1090 | −36.029 | −18.464 | 22.601 | 1.00 | 51.07 |
| ATOM | 7408 | C | ARG | 1090 | −33.639 | −20.038 | 16.458 | 1.00 | 34.36 |
| ATOM | 7409 | O | ARG | 1090 | −33.859 | −19.334 | 15.472 | 1.00 | 34.30 |
| ATOM | 7410 | N | ILE | 1091 | −32.513 | −19.976 | 17.163 | 1.00 | 32.94 |
| ATOM | 7411 | CA | ILE | 1091 | −31.432 | −19.059 | 16.820 | 1.00 | 30.49 |
| ATOM | 7412 | CB | ILE | 1091 | −30.265 | −19.179 | 17.828 | 1.00 | 29.84 |
| ATOM | 7413 | CG2 | ILE | 1091 | −29.087 | −18.304 | 17.382 | 1.00 | 26.75 |
| ATOM | 7414 | CG1 | ILE | 1091 | −30.749 | −18.770 | 19.222 | 1.00 | 27.82 |
| ATOM | 7415 | CD1 | ILE | 1091 | −29.749 | −19.036 | 20.332 | 1.00 | 28.01 |
| ATOM | 7416 | C | ILE | 1091 | −30.904 | −19.311 | 15.418 | 1.00 | 29.96 |
| ATOM | 7417 | O | ILE | 1091 | −30.686 | −18.374 | 14.650 | 1.00 | 30.26 |
| ATOM | 7418 | N | THR | 1092 | −30.711 | −20.580 | 15.078 | 1.00 | 30.58 |
| ATOM | 7419 | CA | THR | 1092 | −30.193 | −20.929 | 13.760 | 1.00 | 31.05 |
| ATOM | 7420 | CB | THR | 1092 | −29.921 | −22.433 | 13.645 | 1.00 | 31.25 |
| ATOM | 7421 | OG1 | THR | 1092 | −28.972 | −22.827 | 14.648 | 1.00 | 31.53 |
| ATOM | 7422 | CG2 | THR | 1092 | −29.357 | −22.760 | 12.269 | 1.00 | 29.03 |
| ATOM | 7423 | C | THR | 1092 | −31.156 | −20.527 | 12.654 | 1.00 | 31.78 |
| ATOM | 7424 | O | THR | 1092 | −30.742 | −20.039 | 11.602 | 1.00 | 32.76 |
| ATOM | 7425 | N | GLU | 1093 | −32.443 | −20.742 | 12.897 | 1.00 | 32.97 |
| ATOM | 7426 | CA | GLU | 1093 | −33.467 | −20.405 | 11.918 | 1.00 | 34.29 |
| ATOM | 7427 | CB | GLU | 1093 | −34.743 | −21.190 | 12.215 | 1.00 | 35.93 |
| ATOM | 7428 | CG | GLU | 1093 | −34.531 | −22.694 | 12.240 | 1.00 | 40.74 |
| ATOM | 7429 | CD | GLU | 1093 | −35.785 | −23.460 | 12.620 | 1.00 | 44.39 |
| ATOM | 7430 | OE1 | GLU | 1093 | −36.357 | −23.178 | 13.698 | 1.00 | 46.36 |
| ATOM | 7431 | OE2 | GLU | 1093 | −36.193 | −24.347 | 11.842 | 1.00 | 45.61 |
| ATOM | 7432 | C | GLU | 1093 | −33.760 | −18.909 | 11.913 | 1.00 | 32.84 |
| ATOM | 7433 | O | GLU | 1093 | −34.238 | −18.366 | 10.915 | 1.00 | 33.67 |
| ATOM | 7434 | N | ALA | 1094 | −33.461 | −18.244 | 13.024 | 1.00 | 32.28 |
| ATOM | 7435 | CA | ALA | 1094 | −33.705 | −16.810 | 13.138 | 1.00 | 30.66 |
| ATOM | 7436 | CB | ALA | 1094 | −33.876 | −16.430 | 14.602 | 1.00 | 31.86 |
| ATOM | 7437 | C | ALA | 1094 | −32.604 | −15.965 | 12.503 | 1.00 | 29.98 |
| ATOM | 7438 | O | ALA | 1094 | −32.881 | −14.912 | 11.927 | 1.00 | 29.52 |
| ATOM | 7439 | N | LEU | 1095 | −31.358 | −16.424 | 12.602 | 1.00 | 27.75 |
| ATOM | 7440 | CA | LEU | 1095 | −30.240 | −15.677 | 12.035 | 1.00 | 26.60 |
| ATOM | 7441 | CB | LEU | 1095 | −28.973 | −15.892 | 12.875 | 1.00 | 27.24 |
| ATOM | 7442 | CG | LEU | 1095 | −28.992 | −15.409 | 14.327 | 1.00 | 28.91 |
| ATOM | 7443 | CD1 | LEU | 1095 | −27.602 | −15.587 | 14.941 | 1.00 | 28.85 |
| ATOM | 7444 | CD2 | LEU | 1095 | −29.400 | −13.948 | 14.386 | 1.00 | 32.53 |
| ATOM | 7445 | C | LEU | 1095 | −29.940 | −16.024 | 10.581 | 1.00 | 25.13 |
| ATOM | 7446 | O | LEU | 1095 | −30.069 | −17.176 | 10.161 | 1.00 | 25.13 |
| ATOM | 7447 | N | ALA | 1096 | −29.544 | −15.016 | 9.813 | 1.00 | 23.38 |
| ATOM | 7448 | CA | ALA | 1096 | −29.194 | −15.221 | 8.417 | 1.00 | 24.18 |
| ATOM | 7449 | CB | ALA | 1096 | −29.358 | −13.921 | 7.636 | 1.00 | 26.66 |
| ATOM | 7450 | C | ALA | 1096 | −27.742 | −15.704 | 8.340 | 1.00 | 23.29 |
| ATOM | 7451 | O | ALA | 1096 | −27.383 | −16.473 | 7.446 | 1.00 | 22.71 |
| ATOM | 7452 | N | ILE | 1097 | −26.910 | −15.253 | 9.278 | 1.00 | 22.57 |
| ATOM | 7453 | CA | ILE | 1097 | −25.507 | −15.662 | 9.295 | 1.00 | 22.02 |
| ATOM | 7454 | CB | ILE | 1097 | −24.638 | −14.773 | 10.231 | 1.00 | 20.30 |
| ATOM | 7455 | CG2 | ILE | 1097 | −24.637 | −13.339 | 9.735 | 1.00 | 18.65 |
| ATOM | 7456 | CG1 | ILE | 1097 | −25.141 | −14.868 | 11.671 | 1.00 | 19.11 |
| ATOM | 7457 | CD1 | ILE | 1097 | −24.232 | −14.180 | 12.681 | 1.00 | 16.71 |
| ATOM | 7458 | C | ILE | 1097 | −25.392 | −17.099 | 9.779 | 1.00 | 21.40 |
| ATOM | 7459 | O | ILE | 1097 | −26.266 | −17.594 | 10.489 | 1.00 | 20.96 |
| ATOM | 7460 | N | PRO | 1098 | −24.313 | −17.793 | 9.391 | 1.00 | 21.86 |
| ATOM | 7461 | CD | PRO | 1098 | −23.284 | −17.422 | 8.409 | 1.00 | 22.08 |
| ATOM | 7462 | CA | PRO | 1098 | −24.157 | −19.180 | 9.832 | 1.00 | 22.32 |
| ATOM | 7463 | CB | PRO | 1098 | −23.004 | −19.700 | 8.968 | 1.00 | 21.93 |
| ATOM | 7464 | CG | PRO | 1098 | −22.236 | −18.469 | 8.626 | 1.00 | 24.68 |
| ATOM | 7465 | C | PRO | 1098 | −23.896 | −19.316 | 11.329 | 1.00 | 22.49 |
| ATOM | 7466 | O | PRO | 1098 | −23.160 | −18.528 | 11.933 | 1.00 | 21.97 |
| ATOM | 7467 | N | VAL | 1099 | −24.527 | −20.317 | 11.930 | 1.00 | 21.40 |
| ATOM | 7468 | CA | VAL | 1099 | −24.368 | −20.569 | 13.351 | 1.00 | 20.38 |
| ATOM | 7469 | CB | VAL | 1099 | −25.735 | −20.667 | 14.048 | 1.00 | 21.59 |
| ATOM | 7470 | CG1 | VAL | 1099 | −25.543 | −21.007 | 15.518 | 1.00 | 20.95 |
| ATOM | 7471 | CG2 | VAL | 1099 | −26.485 | −19.348 | 13.902 | 1.00 | 20.13 |
| ATOM | 7472 | C | VAL | 1099 | −23.593 | −21.862 | 13.568 | 1.00 | 20.91 |
| ATOM | 7473 | O | VAL | 1099 | −24.009 | −22.929 | 13.119 | 1.00 | 21.10 |
| ATOM | 7474 | N | ILE | 1100 | −22.459 | −21.749 | 14.252 | 1.00 | 20.43 |
| ATOM | 7475 | CA | ILE | 1100 | −21.606 | −22.897 | 14.553 | 1.00 | 19.39 |
| ATOM | 7476 | CB | ILE | 1100 | −20.106 | −22.533 | 14.393 | 1.00 | 18.61 |
| ATOM | 7477 | CG2 | ILE | 1100 | −19.235 | −23.709 | 14.814 | 1.00 | 21.73 |
| ATOM | 7478 | CG1 | ILE | 1100 | −19.807 | −22.154 | 12.939 | 1.00 | 19.30 |
| ATOM | 7479 | CD1 | ILE | 1100 | −18.369 | −21.678 | 12.698 | 1.00 | 18.01 |
| ATOM | 7480 | C | ILE | 1100 | −21.862 | −23.344 | 15.992 | 1.00 | 17.92 |
| ATOM | 7481 | O | ILE | 1100 | −21.811 | −22.541 | 16.925 | 1.00 | 19.15 |
| ATOM | 7482 | N | GLY | 1101 | −22.126 | −24.629 | 16.179 | 1.00 | 17.28 |
| ATOM | 7483 | CA | GLY | 1101 | −22.392 | −25.087 | 17.521 | 1.00 | 15.61 |
| ATOM | 7484 | C | GLY | 1101 | −21.405 | −26.058 | 18.134 | 1.00 | 16.33 |
| ATOM | 7485 | O | GLY | 1101 | −20.607 | −26.692 | 17.449 | 1.00 | 14.73 |
| ATOM | 7486 | N | ILE | 1102 | −21.450 | −26.126 | 19.457 | 1.00 | 16.82 |
| ATOM | 7487 | CA | ILE | 1102 | −20.642 | −27.055 | 20.228 | 1.00 | 17.43 |
| ATOM | 7488 | CB | ILE | 1102 | −19.213 | −26.493 | 20.587 | 1.00 | 15.75 |
| ATOM | 7489 | CG2 | ILE | 1102 | −19.297 | −25.103 | 21.214 | 1.00 | 16.29 |
| ATOM | 7490 | CG1 | ILE | 1102 | −18.514 | −27.477 | 21.519 | 1.00 | 17.38 |
| ATOM | 7491 | CD1 | ILE | 1102 | −17.048 | −27.149 | 21.769 | 1.00 | 17.39 |
| ATOM | 7492 | C | ILE | 1102 | −21.486 | −27.320 | 21.466 | 1.00 | 16.49 |
| ATOM | 7493 | O | ILE | 1102 | −21.652 | −26.457 | 22.324 | 1.00 | 18.86 |
| ATOM | 7494 | N | GLY | 1103 | −22.064 | −28.515 | 21.525 | 1.00 | 18.98 |
| ATOM | 7495 | CA | GLY | 1103 | −22.913 | −28.857 | 22.649 | 1.00 | 19.60 |
| ATOM | 7496 | C | GLY | 1103 | −24.275 | −28.209 | 22.470 | 1.00 | 21.48 |
| ATOM | 7497 | O | GLY | 1103 | −24.969 | −27.921 | 23.443 | 1.00 | 22.27 |
| ATOM | 7498 | N | ALA | 1104 | −24.652 | −27.972 | 21.217 | 1.00 | 22.82 |
| ATOM | 7499 | CA | ALA | 1104 | −25.935 | −27.353 | 20.908 | 1.00 | 25.45 |
| ATOM | 7500 | CB | ALA | 1104 | −25.716 | −25.988 | 20.259 | 1.00 | 25.68 |
| ATOM | 7501 | C | ALA | 1104 | −26.771 | −28.236 | 19.987 | 1.00 | 25.66 |
| ATOM | 7502 | O | ALA | 1104 | −27.830 | −27.820 | 19.515 | 1.00 | 26.77 |
| ATOM | 7503 | N | GLY | 1105 | −26.293 | −29.448 | 19.722 | 1.00 | 26.62 |
| ATOM | 7504 | CA | GLY | 1105 | −27.026 | −30.351 | 18.852 | 1.00 | 26.39 |
| ATOM | 7505 | C | GLY | 1105 | −26.597 | −30.240 | 17.400 | 1.00 | 26.35 |
| ATOM | 7506 | O | GLY | 1105 | −25.665 | −29.504 | 17.087 | 1.00 | 27.27 |
| ATOM | 7507 | N | ASN | 1106 | −27.276 | −30.966 | 16.514 | 1.00 | 24.67 |
| ATOM | 7508 | CA | ASN | 1106 | −26.946 | −30.947 | 15.095 | 1.00 | 25.28 |
| ATOM | 7509 | CB | ASN | 1106 | −27.003 | −32.363 | 14.513 | 1.00 | 27.03 |
| ATOM | 7510 | CG | ASN | 1106 | −28.406 | −32.957 | 14.543 | 1.00 | 28.79 |
| ATOM | 7511 | OD1 | ASN | 1106 | −28.706 | −33.901 | 13.810 | 1.00 | 30.36 |
| ATOM | 7512 | ND2 | ASN | 1106 | −29.267 | −32.414 | 15.398 | 1.00 | 26.73 |
| ATOM | 7513 | C | ASN | 1106 | −27.851 | −30.036 | 14.273 | 1.00 | 25.16 |
| ATOM | 7514 | O | ASN | 1106 | −27.889 | −30.133 | 13.046 | 1.00 | 26.21 |
| ATOM | 7515 | N | VAL | 1107 | −28.563 | −29.143 | 14.949 | 1.00 | 26.94 |
| ATOM | 7516 | CA | VAL | 1107 | −29.476 | −28.217 | 14.287 | 1.00 | 27.66 |
| ATOM | 7517 | CB | VAL | 1107 | −30.583 | −27.753 | 15.255 | 1.00 | 29.34 |
| ATOM | 7518 | CG1 | VAL | 1107 | −31.608 | −26.904 | 14.507 | 1.00 | 33.27 |
| ATOM | 7519 | CG2 | VAL | 1107 | −31.250 | −28.962 | 15.898 | 1.00 | 30.08 |
| ATOM | 7520 | C | VAL | 1107 | −28.751 | −26.984 | 13.761 | 1.00 | 27.29 |
| ATOM | 7521 | O | VAL | 1107 | −29.274 | −26.255 | 12.915 | 1.00 | 26.10 |
| ATOM | 7522 | N | THR | 1108 | −27.547 | −26.746 | 14.267 | 1.00 | 24.64 |
| ATOM | 7523 | CA | THR | 1108 | −26.769 | −25.592 | 13.840 | 1.00 | 22.37 |
| ATOM | 7524 | CB | THR | 1108 | −25.594 | −25.337 | 14.805 | 1.00 | 21.49 |
| ATOM | 7525 | OG1 | THR | 1108 | −24.814 | −26.532 | 14.944 | 1.00 | 20.03 |
| ATOM | 7526 | CG2 | THR | 1108 | −26.116 | −24.918 | 16.171 | 1.00 | 20.67 |
| ATOM | 7527 | C | THR | 1108 | −26.242 | −25.787 | 12.424 | 1.00 | 22.32 |
| ATOM | 7528 | O | THR | 1108 | −26.276 | −26.891 | 11.888 | 1.00 | 23.83 |
| ATOM | 7529 | N | ASP | 1109 | −25.764 | −24.705 | 11.820 | 1.00 | 21.15 |
| ATOM | 7530 | CA | ASP | 1109 | −25.241 | −24.746 | 10.461 | 1.00 | 21.07 |
| ATOM | 7531 | CB | ASP | 1109 | −25.122 | −23.326 | 9.918 | 1.00 | 22.92 |
| ATOM | 7532 | CG | ASP | 1109 | −26.450 | −22.605 | 9.904 | 1.00 | 25.19 |
| ATOM | 7533 | OD1 | ASP | 1109 | −27.371 | −23.091 | 9.220 | 1.00 | 22.36 |
| ATOM | 7534 | OD2 | ASP | 1109 | −26.577 | −21.565 | 10.589 | 1.00 | 26.54 |
| ATOM | 7535 | C | ASP | 1109 | −23.889 | −25.432 | 10.389 | 1.00 | 20.97 |
| ATOM | 7536 | O | ASP | 1109 | −23.511 | −25.975 | 9.353 | 1.00 | 20.53 |
| ATOM | 7537 | N | GLY | 1110 | −23.161 | −25.396 | 11.498 | 1.00 | 21.29 |
| ATOM | 7538 | CA | GLY | 1110 | −21.855 | −26.020 | 11.541 | 1.00 | 19.83 |
| ATOM | 7539 | C | GLY | 1110 | −21.532 | −26.555 | 12.920 | 1.00 | 18.45 |
| ATOM | 7540 | O | GLY | 1110 | −22.321 | −26.421 | 13.856 | 1.00 | 16.61 |
| ATOM | 7541 | N | GLN | 1111 | −20.360 | −27.169 | 13.046 | 1.00 | 18.75 |
| ATOM | 7542 | CA | GLN | 1111 | −19.920 | −27.725 | 14.315 | 1.00 | 18.97 |
| ATOM | 7543 | CB | GLN | 1111 | −20.030 | −29.253 | 14.300 | 1.00 | 20.40 |
| ATOM | 7544 | CG | GLN | 1111 | −21.434 | −29.822 | 14.149 | 1.00 | 21.52 |
| ATOM | 7545 | CD | GLN | 1111 | −22.353 | −29.426 | 15.281 | 1.00 | 22.47 |
| ATOM | 7546 | OE1 | GLN | 1111 | −21.950 | −29.393 | 16.448 | 1.00 | 25.39 |
| ATOM | 7547 | NE2 | GLN | 1111 | −23.607 | −29.136 | 14.946 | 1.00 | 23.89 |
| ATOM | 7548 | C | GLN | 1111 | −18.462 | −27.364 | 14.596 | 1.00 | 17.61 |
| ATOM | 7549 | O | GLN | 1111 | −17.697 | −27.072 | 13.679 | 1.00 | 16.60 |
| ATOM | 7550 | N | ILE | 1112 | −18.089 | −27.389 | 15.871 | 1.00 | 17.79 |
| ATOM | 7551 | CA | ILE | 1112 | −16.716 | −27.131 | 16.276 | 1.00 | 18.54 |
| ATOM | 7552 | CB | ILE | 1112 | −16.454 | −25.623 | 16.531 | 1.00 | 18.72 |
| ATOM | 7553 | CG2 | ILE | 1112 | −17.191 | −25.146 | 17.786 | 1.00 | 18.50 |
| ATOM | 7554 | CG1 | ILE | 1112 | −14.945 | −25.390 | 16.671 | 1.00 | 18.93 |
| ATOM | 7555 | CD1 | ILE | 1112 | −14.535 | −23.906 | 16.715 | 1.00 | 20.59 |
| ATOM | 7556 | C | ILE | 1112 | −16.417 | −27.941 | 17.540 | 1.00 | 21.11 |
| ATOM | 7557 | O | ILE | 1112 | −17.319 | −28.264 | 18.321 | 1.00 | 20.41 |
| ATOM | 7558 | N | LEU | 1113 | −15.148 | −28.288 | 17.720 | 1.00 | 22.35 |
| ATOM | 7559 | CA | LEU | 1113 | −14.726 | −29.054 | 18.880 | 1.00 | 23.98 |
| ATOM | 7560 | CB | LEU | 1113 | −15.130 | −30.515 | 18.700 | 1.00 | 28.74 |
| ATOM | 7561 | CG | LEU | 1113 | −15.427 | −31.304 | 19.980 | 1.00 | 32.72 |
| ATOM | 7562 | CD1 | LEU | 1113 | −16.778 | −30.878 | 20.556 | 1.00 | 32.53 |
| ATOM | 7563 | CD2 | LEU | 1113 | −15.441 | −32.784 | 19.648 | 1.00 | 35.33 |
| ATOM | 7564 | C | LEU | 1113 | −13.212 | −28.947 | 18.990 | 1.00 | 23.34 |
| ATOM | 7565 | O | LEU | 1113 | −12.534 | −28.716 | 17.983 | 1.00 | 22.61 |
| ATOM | 7566 | N | VAL | 1114 | −12.687 | −29.104 | 20.205 | 1.00 | 21.36 |
| ATOM | 7567 | CA | VAL | 1114 | −11.246 | −29.044 | 20.440 | 1.00 | 19.62 |
| ATOM | 7568 | CB | VAL | 1114 | −10.928 | −28.996 | 21.966 | 1.00 | 20.48 |
| ATOM | 7569 | CG1 | VAL | 1114 | −9.434 | −28.834 | 22.187 | 1.00 | 20.97 |
| ATOM | 7570 | CG2 | VAL | 1114 | −11.702 | −27.870 | 22.620 | 1.00 | 20.82 |
| ATOM | 7571 | C | VAL | 1114 | −10.628 | −30.304 | 19.851 | 1.00 | 18.23 |
| ATOM | 7572 | O | VAL | 1114 | −11.017 | −31.419 | 20.210 | 1.00 | 18.70 |
| ATOM | 7573 | N | MET | 1115 | −9.676 | −30.137 | 18.943 | 1.00 | 16.13 |
| ATOM | 7574 | CA | MET | 1115 | −9.041 | −31.287 | 18.305 | 1.00 | 14.70 |
| ATOM | 7575 | CB | MET | 1115 | −7.900 | −30.830 | 17.390 | 1.00 | 14.14 |
| ATOM | 7576 | CG | MET | 1115 | −6.823 | −30.025 | 18.095 | 1.00 | 15.65 |
| ATOM | 7577 | SD | MET | 1115 | −5.242 | −30.356 | 17.297 | 1.00 | 13.45 |
| ATOM | 7578 | CE | MET | 1115 | −4.722 | −31.861 | 18.197 | 1.00 | 11.23 |
| ATOM | 7579 | C | MET | 1115 | −8.492 | −32.315 | 19.287 | 1.00 | 13.25 |
| ATOM | 7580 | O | MET | 1115 | −8.533 | −33.510 | 19.019 | 1.00 | 16.95 |
| ATOM | 7581 | N | HIS | 1116 | −7.969 | −31.866 | 20.421 | 1.00 | 12.84 |
| ATOM | 7582 | CA | HIS | 1116 | −7.414 | −32.828 | 21.377 | 1.00 | 14.12 |
| ATOM | 7583 | CB | HIS | 1116 | −6.724 | −32.107 | 22.521 | 1.00 | 13.42 |
| ATOM | 7584 | CG | HIS | 1116 | −5.516 | −31.336 | 22.084 | 1.00 | 14.34 |
| ATOM | 7585 | CD2 | HIS | 1116 | −5.403 | −30.121 | 21.494 | 1.00 | 12.56 |
| ATOM | 7586 | ND1 | HIS | 1116 | −4.237 | −31.839 | 22.165 | 1.00 | 14.51 |
| ATOM | 7587 | CE1 | HIS | 1116 | −3.384 | −30.971 | 21.646 | 1.00 | 12.60 |
| ATOM | 7588 | NE2 | HIS | 1116 | −4.067 | −29.919 | 21.230 | 1.00 | 19.44 |
| ATOM | 7589 | C | HIS | 1116 | −8.476 | −33.784 | 21.920 | 1.00 | 14.04 |
| ATOM | 7590 | O | HIS | 1116 | −8.168 | −34.939 | 22.232 | 1.00 | 16.57 |
| ATOM | 7591 | N | ASP | 1117 | −9.717 | −33.318 | 22.041 | 1.00 | 12.82 |
| ATOM | 7592 | CA | ASP | 1117 | −10.794 | −34.176 | 22.536 | 1.00 | 15.57 |
| ATOM | 7593 | CB | ASP | 1117 | −12.014 | −33.359 | 22.984 | 1.00 | 15.67 |
| ATOM | 7594 | CG | ASP | 1117 | −11.741 | −32.484 | 24.179 | 1.00 | 15.82 |
| ATOM | 7595 | OD1 | ASP | 1117 | −10.803 | −32.772 | 24.947 | 1.00 | 15.40 |
| ATOM | 7596 | OD2 | ASP | 1117 | −12.486 | −31.500 | 24.364 | 1.00 | 16.01 |
| ATOM | 7597 | C | ASP | 1117 | −11.256 | −35.108 | 21.424 | 1.00 | 17.95 |
| ATOM | 7598 | O | ASP | 1117 | −11.438 | −36.306 | 21.633 | 1.00 | 18.83 |
| ATOM | 7599 | N | ALA | 1118 | −11.442 | −34.533 | 20.237 | 1.00 | 18.76 |
| ATOM | 7600 | CA | ALA | 1118 | −11.927 | −35.267 | 19.073 | 1.00 | 20.93 |
| ATOM | 7601 | CB | ALA | 1118 | −12.291 | −34.286 | 17.965 | 1.00 | 21.95 |
| ATOM | 7602 | C | ALA | 1118 | −10.983 | −36.323 | 18.525 | 1.00 | 22.38 |
| ATOM | 7603 | O | ALA | 1118 | −11.420 | −37.225 | 17.815 | 1.00 | 23.45 |
| ATOM | 7604 | N | PHE | 1119 | −9.698 | −36.218 | 18.846 | 1.00 | 23.93 |
| ATOM | 7605 | CA | PHE | 1119 | −8.739 | −37.179 | 18.340 | 1.00 | 27.36 |
| ATOM | 7606 | CB | PHE | 1119 | −7.670 | −36.470 | 17.521 | 1.00 | 32.16 |
| ATOM | 7607 | CG | PHE | 1119 | −8.223 | −35.714 | 16.375 | 1.00 | 35.71 |
| ATOM | 7608 | CD1 | PHE | 1119 | −9.075 | −36.337 | 15.474 | 1.00 | 37.69 |
| ATOM | 7609 | CD2 | PHE | 1119 | −7.922 | −34.371 | 16.203 | 1.00 | 38.44 |
| ATOM | 7610 | CE1 | PHE | 1119 | −9.620 | −35.633 | 14.420 | 1.00 | 40.51 |
| ATOM | 7611 | CE2 | PHE | 1119 | −8.463 | −33.660 | 15.152 | 1.00 | 39.76 |
| ATOM | 7612 | CZ | PHE | 1119 | −9.314 | −34.287 | 14.256 | 1.00 | 41.23 |
| ATOM | 7613 | C | PHE | 1119 | −8.086 | −38.050 | 19.379 | 1.00 | 26.58 |
| ATOM | 7614 | O | PHE | 1119 | −6.942 | −38.460 | 19.212 | 1.00 | 30.89 |
| ATOM | 7615 | N | GLY | 1120 | −8.817 | −38.318 | 20.454 | 1.00 | 24.48 |
| ATOM | 7616 | CA | GLY | 1120 | −8.327 | −39.186 | 21.505 | 1.00 | 24.13 |
| ATOM | 7617 | C | GLY | 1120 | −7.005 | −38.851 | 22.160 | 1.00 | 23.63 |
| ATOM | 7618 | O | GLY | 1120 | −6.283 | −39.758 | 22.581 | 1.00 | 22.16 |
| ATOM | 7619 | N | ILE | 1121 | −6.680 | −37.565 | 22.252 | 1.00 | 20.46 |
| ATOM | 7620 | CA | ILE | 1121 | −5.441 | −37.145 | 22.900 | 1.00 | 19.00 |
| ATOM | 7621 | CB | ILE | 1121 | −4.836 | −35.889 | 22.225 | 1.00 | 16.75 |
| ATOM | 7622 | CG2 | ILE | 1121 | −3.614 | −35.423 | 22.995 | 1.00 | 16.32 |
| ATOM | 7623 | CG1 | ILE | 1121 | −4.456 | −36.203 | 20.777 | 1.00 | 15.62 |
| ATOM | 7624 | CD1 | ILE | 1121 | −4.238 | −34.960 | 19.921 | 1.00 | 14.00 |
| ATOM | 7625 | C | ILE | 1121 | −5.758 | −36.832 | 24.359 | 1.00 | 19.57 |
| ATOM | 7626 | O | ILE | 1121 | −5.100 | −37.335 | 25.265 | 1.00 | 17.67 |
| ATOM | 7627 | N | THR | 1122 | −6.774 | −36.009 | 24.591 | 1.00 | 20.23 |
| ATOM | 7628 | CA | THR | 1122 | −7.148 | −35.671 | 25.958 | 1.00 | 23.81 |
| ATOM | 7629 | CB | THR | 1122 | −8.387 | −34.773 | 25.979 | 1.00 | 24.49 |
| ATOM | 7630 | OG1 | THR | 1122 | −9.425 | −35.373 | 25.195 | 1.00 | 28.23 |
| ATOM | 7631 | CG2 | THR | 1122 | −8.053 | −33.409 | 25.402 | 1.00 | 18.95 |
| ATOM | 7632 | C | THR | 1122 | −7.443 | −36.940 | 26.751 | 1.00 | 26.71 |
| ATOM | 7633 | O | THR | 1122 | −8.052 | −37.872 | 26.228 | 1.00 | 26.64 |
| ATOM | 7634 | N | GLY | 1123 | −7.002 | −36.962 | 28.005 | 1.00 | 30.43 |
| ATOM | 7635 | CA | GLY | 1123 | −7.207 | −38.113 | 28.870 | 1.00 | 37.27 |
| ATOM | 7636 | C | GLY | 1123 | −8.332 | −39.059 | 28.484 | 1.00 | 39.65 |
| ATOM | 7637 | O | GLY | 1123 | −8.205 | −39.847 | 27.544 | 1.00 | 41.64 |
| ATOM | 7638 | N | GLY | 1124 | −9.441 | −38.986 | 29.211 | 1.00 | 40.46 |
| ATOM | 7639 | CA | GLY | 1124 | −10.569 | −39.852 | 28.920 | 1.00 | 38.97 |
| ATOM | 7640 | C | GLY | 1124 | −11.873 | −39.208 | 29.340 | 1.00 | 37.18 |
| ATOM | 7641 | O | GLY | 1124 | −12.953 | −39.759 | 29.128 | 1.00 | 38.03 |
| ATOM | 7642 | N | HIS | 1125 | −11.763 | −38.026 | 29.932 | 1.00 | 36.46 |
| ATOM | 7643 | CA | HIS | 1125 | −12.924 | −37.290 | 30.399 | 1.00 | 35.97 |
| ATOM | 7644 | CB | HIS | 1125 | −12.653 | −36.726 | 31.792 | 1.00 | 37.38 |
| ATOM | 7645 | CG | HIS | 1125 | −12.325 | −37.771 | 32.812 | 1.00 | 39.81 |
| ATOM | 7646 | CD2 | HIS | 1125 | −11.211 | −37.973 | 33.554 | 1.00 | 40.42 |
| ATOM | 7647 | ND1 | HIS | 1125 | −13.207 | −38.769 | 33.164 | 1.00 | 40.29 |
| ATOM | 7648 | CE1 | HIS | 1125 | −12.651 | −39.541 | 34.081 | 1.00 | 40.40 |
| ATOM | 7649 | NE2 | HIS | 1125 | −11.440 | −39.080 | 34.335 | 1.00 | 41.75 |
| ATOM | 7650 | C | HIS | 1125 | −13.291 | −36.152 | 29.456 | 1.00 | 34.78 |
| ATOM | 7651 | O | HIS | 1125 | −13.410 | −35.000 | 29.880 | 1.00 | 36.44 |
| ATOM | 7652 | N | ILE | 1126 | −13.466 | −36.466 | 28.178 | 1.00 | 31.25 |
| ATOM | 7653 | CA | ILE | 1126 | −13.835 | −35.435 | 27.215 | 1.00 | 28.48 |
| ATOM | 7654 | CB | ILE | 1126 | −13.804 | −35.963 | 25.767 | 1.00 | 26.91 |
| ATOM | 7655 | CG2 | ILE | 1126 | −12.394 | −36.360 | 25.388 | 1.00 | 26.98 |
| ATOM | 7656 | CG1 | ILE | 1126 | −14.768 | −37.138 | 25.614 | 1.00 | 25.19 |
| ATOM | 7657 | CD1 | ILE | 1126 | −14.926 | −37.612 | 24.194 | 1.00 | 22.08 |
| ATOM | 7658 | C | ILE | 1126 | −15.249 | −34.961 | 27.540 | 1.00 | 28.29 |
| ATOM | 7659 | O | ILE | 1126 | −16.044 | −35.702 | 28.122 | 1.00 | 27.48 |
| ATOM | 7660 | N | PRO | 1127 | −15.586 | −33.719 | 27.168 | 1.00 | 27.02 |
| ATOM | 7661 | CD | PRO | 1127 | −14.838 | −32.750 | 26.350 | 1.00 | 26.06 |
| ATOM | 7662 | CA | PRO | 1127 | −16.936 | −33.232 | 27.470 | 1.00 | 27.27 |
| ATOM | 7663 | CB | PRO | 1127 | −16.946 | −31.831 | 26.853 | 1.00 | 27.40 |
| ATOM | 7664 | CG | PRO | 1127 | −15.944 | −31.927 | 25.749 | 1.00 | 29.05 |
| ATOM | 7665 | C | PRO | 1127 | −18.045 | −34.136 | 26.925 | 1.00 | 27.42 |
| ATOM | 7666 | O | PRO | 1127 | −17.839 | −34.877 | 25.962 | 1.00 | 25.23 |
| ATOM | 7667 | N | LYS | 1128 | −19.213 | −34.081 | 27.559 | 1.00 | 28.67 |
| ATOM | 7668 | CA | LYS | 1128 | −20.354 | −34.895 | 27.145 | 1.00 | 29.40 |
| ATOM | 7669 | CB | LYS | 1128 | −21.571 | −34.609 | 28.033 | 1.00 | 32.06 |
| ATOM | 7670 | CG | LYS | 1128 | −21.569 | −35.326 | 29.376 | 1.00 | 37.86 |
| ATOM | 7671 | CD | LYS | 1128 | −20.410 | −34.884 | 30.254 | 1.00 | 41.77 |
| ATOM | 7672 | CE | LYS | 1128 | −20.424 | −35.614 | 31.586 | 1.00 | 42.92 |
| ATOM | 7673 | NZ | LYS | 1128 | −19.313 | −35.170 | 32.465 | 1.00 | 45.73 |
| ATOM | 7674 | C | LYS | 1128 | −20.746 | −34.674 | 25.691 | 1.00 | 28.13 |
| ATOM | 7675 | O | LYS | 1128 | −21.216 | −35.595 | 25.020 | 1.00 | 28.71 |
| ATOM | 7676 | N | PHE | 1129 | −20.549 | −33.452 | 25.208 | 1.00 | 25.91 |
| ATOM | 7677 | CA | PHE | 1129 | −20.902 | −33.098 | 23.834 | 1.00 | 24.94 |
| ATOM | 7678 | CB | PHE | 1129 | −21.220 | −31.605 | 23.761 | 1.00 | 24.88 |
| ATOM | 7679 | CG | PHE | 1129 | −20.113 | −30.717 | 24.268 | 1.00 | 25.53 |
| ATOM | 7680 | CD1 | PHE | 1129 | −18.967 | −30.501 | 23.508 | 1.00 | 25.50 |
| ATOM | 7681 | CD2 | PHE | 1129 | −20.218 | −30.098 | 25.508 | 1.00 | 26.10 |
| ATOM | 7682 | CE1 | PHE | 1129 | −17.938 | −29.678 | 23.976 | 1.00 | 24.44 |
| ATOM | 7683 | CE2 | PHE | 1129 | −19.196 | −29.272 | 25.989 | 1.00 | 28.35 |
| ATOM | 7684 | CZ | PHE | 1129 | −18.055 | −29.062 | 25.219 | 1.00 | 25.02 |
| ATOM | 7685 | C | PHE | 1129 | −19.830 | −33.444 | 22.809 | 1.00 | 24.51 |
| ATOM | 7686 | O | PHE | 1129 | −20.017 | −33.241 | 21.608 | 1.00 | 25.21 |
| ATOM | 7687 | N | ALA | 1130 | −18.711 | −33.975 | 23.285 | 1.00 | 22.79 |
| ATOM | 7688 | CA | ALA | 1130 | −17.611 | −34.322 | 22.400 | 1.00 | 22.40 |
| ATOM | 7689 | CB | ALA | 1130 | −16.285 | −33.933 | 23.047 | 1.00 | 22.61 |
| ATOM | 7690 | C | ALA | 1130 | −17.587 | −35.793 | 22.030 | 1.00 | 22.33 |
| ATOM | 7691 | O | ALA | 1130 | −18.222 | −36.627 | 22.674 | 1.00 | 23.47 |
| ATOM | 7692 | N | LYS | 1131 | −16.824 | −36.105 | 20.992 | 1.00 | 21.42 |
| ATOM | 7693 | CA | LYS | 1131 | −16.699 | −37.473 | 20.534 | 1.00 | 21.01 |
| ATOM | 7694 | CB | LYS | 1131 | −17.667 | −37.723 | 19.374 | 1.00 | 21.85 |
| ATOM | 7695 | CG | LYS | 1131 | −17.613 | −39.131 | 18.807 | 1.00 | 25.99 |
| ATOM | 7696 | CD | LYS | 1131 | −18.660 | −39.339 | 17.722 | 1.00 | 27.36 |
| ATOM | 7697 | CE | LYS | 1131 | −18.521 | −40.719 | 17.096 | 1.00 | 28.45 |
| ATOM | 7698 | NZ | LYS | 1131 | −19.590 | −41.000 | 16.100 | 1.00 | 30.79 |
| ATOM | 7699 | C | LYS | 1131 | −15.272 | −37.770 | 20.099 | 1.00 | 20.50 |
| ATOM | 7700 | O | LYS | 1131 | −14.615 | −36.946 | 19.456 | 1.00 | 19.85 |
| ATOM | 7701 | N | ASN | 1132 | −14.788 | −38.946 | 20.475 | 1.00 | 18.18 |
| ATOM | 7702 | CA | ASN | 1132 | −13.450 | −39.373 | 20.100 | 1.00 | 19.09 |
| ATOM | 7703 | CB | ASN | 1132 | −12.860 | −40.286 | 21.178 | 1.00 | 18.83 |
| ATOM | 7704 | CG | ASN | 1132 | −11.520 | −40.864 | 20.775 | 1.00 | 18.02 |
| ATOM | 7705 | OD1 | ASN | 1132 | −11.079 | −40.694 | 19.642 | 1.00 | 17.99 |
| ATOM | 7706 | ND2 | ASN | 1132 | −10.872 | −41.557 | 21.696 | 1.00 | 17.64 |
| ATOM | 7707 | C | ASN | 1132 | −13.563 | −40.141 | 18.786 | 1.00 | 18.64 |
| ATOM | 7708 | O | ASN | 1132 | −13.884 | −41.332 | 18.781 | 1.00 | 19.00 |
| ATOM | 7709 | N | PHE | 1133 | −13.296 | −39.460 | 17.678 | 1.00 | 18.32 |
| ATOM | 7710 | CA | PHE | 1133 | −13.376 | −40.079 | 16.361 | 1.00 | 18.77 |
| ATOM | 7711 | CB | PHE | 1133 | −13.485 | −39.008 | 15.268 | 1.00 | 19.12 |
| ATOM | 7712 | CG | PHE | 1133 | −14.774 | −38.242 | 15.297 | 1.00 | 19.88 |
| ATOM | 7713 | CD1 | PHE | 1133 | −14.925 | −37.142 | 16.127 | 1.00 | 20.42 |
| ATOM | 7714 | CD2 | PHE | 1133 | −15.858 | −38.656 | 14.531 | 1.00 | 21.73 |
| ATOM | 7715 | CE1 | PHE | 1133 | −16.144 | −36.453 | 16.200 | 1.00 | 22.12 |
| ATOM | 7716 | CE2 | PHE | 1133 | −17.086 | −37.978 | 14.592 | 1.00 | 22.46 |
| ATOM | 7717 | CZ | PHE | 1133 | −17.225 | −36.879 | 15.431 | 1.00 | 21.23 |
| ATOM | 7718 | C | PHE | 1133 | −12.208 | −40.999 | 16.036 | 1.00 | 18.83 |
| ATOM | 7719 | O | PHE | 1133 | −12.314 | −41.836 | 15.144 | 1.00 | 19.05 |
| ATOM | 7720 | N | LEU | 1134 | −11.094 | −40.849 | 16.749 | 1.00 | 17.75 |
| ATOM | 7721 | CA | LEU | 1134 | −9.920 | −41.686 | 16.488 | 1.00 | 18.27 |
| ATOM | 7722 | CB | LEU | 1134 | −8.691 | −41.149 | 17.230 | 1.00 | 17.93 |
| ATOM | 7723 | CG | LEU | 1134 | −7.395 | −41.958 | 17.089 | 1.00 | 19.52 |
| ATOM | 7724 | CD1 | LEU | 1134 | −6.983 | −41.987 | 15.625 | 1.00 | 18.91 |
| ATOM | 7725 | CD2 | LEU | 1134 | −6.271 | −41.335 | 17.936 | 1.00 | 16.96 |
| ATOM | 7726 | C | LEU | 1134 | −10.170 | −43.126 | 16.908 | 1.00 | 19.68 |
| ATOM | 7727 | O | LEU | 1134 | −9.860 | −44.059 | 16.167 | 1.00 | 17.88 |
| ATOM | 7728 | N | ALA | 1135 | −10.727 | −43.297 | 18.101 | 1.00 | 22.36 |
| ATOM | 7729 | CA | ALA | 1135 | −11.027 | −44.623 | 18.617 | 1.00 | 27.76 |
| ATOM | 7730 | CB | ALA | 1135 | −11.775 | −44.513 | 19.934 | 1.00 | 26.50 |
| ATOM | 7731 | C | ALA | 1135 | −11.867 | −45.387 | 17.603 | 1.00 | 32.49 |
| ATOM | 7732 | O | ALA | 1135 | −11.683 | −46.590 | 17.403 | 1.00 | 33.61 |
| ATOM | 7733 | N | GLU | 1136 | −12.785 | −44.668 | 16.964 | 1.00 | 36.50 |
| ATOM | 7734 | CA | GLU | 1136 | −13.676 | −45.240 | 15.963 | 1.00 | 40.36 |
| ATOM | 7735 | CB | GLU | 1136 | −14.606 | −44.158 | 15.403 | 1.00 | 43.73 |
| ATOM | 7736 | CG | GLU | 1136 | −15.434 | −43.403 | 16.441 | 1.00 | 48.10 |
| ATOM | 7737 | CD | GLU | 1136 | −16.536 | −44.250 | 17.054 | 1.00 | 50.51 |
| ATOM | 7738 | OE1 | GLU | 1136 | −16.222 | −45.167 | 17.846 | 1.00 | 51.66 |
| ATOM | 7739 | OE2 | GLU | 1136 | −17.719 | −44.002 | 16.735 | 1.00 | 51.42 |
| ATOM | 7740 | C | GLU | 1136 | −12.865 | −45.831 | 14.820 | 1.00 | 41.02 |
| ATOM | 7741 | O | GLU | 1136 | −12.942 | −47.026 | 14.544 | 1.00 | 42.71 |
| ATOM | 7742 | N | THR | 1137 | −12.091 | −44.979 | 14.155 | 1.00 | 39.55 |
| ATOM | 7743 | CA | THR | 1137 | −11.273 | −45.406 | 13.033 | 1.00 | 38.43 |
| ATOM | 7744 | CB | THR | 1137 | −10.911 | −44.218 | 12.124 | 1.00 | 39.66 |
| ATOM | 7745 | OG1 | THR | 1137 | −9.779 | −44.569 | 11.313 | 1.00 | 41.87 |
| ATOM | 7746 | CG2 | THR | 1137 | −10.595 | −42.973 | 12.953 | 1.00 | 37.36 |
| ATOM | 7747 | C | THR | 1137 | −9.993 | −46.105 | 13.474 | 1.00 | 37.92 |
| ATOM | 7748 | O | THR | 1137 | −9.965 | −47.326 | 13.641 | 1.00 | 40.29 |
| ATOM | 7749 | N | GLY | 1138 | −8.932 | −45.333 | 13.659 | 1.00 | 33.87 |
| ATOM | 7750 | CA | GLY | 1138 | −7.669 | −45.905 | 14.084 | 1.00 | 31.20 |
| ATOM | 7751 | C | GLY | 1138 | −6.522 | −45.066 | 13.574 | 1.00 | 29.12 |
| ATOM | 7752 | O | GLY | 1138 | −5.366 | −45.257 | 13.951 | 1.00 | 27.41 |
| ATOM | 7753 | N | ASP | 1139 | −6.865 | −44.119 | 12.711 | 1.00 | 25.34 |
| ATOM | 7754 | CA | ASP | 1139 | −5.893 | −43.224 | 12.105 | 1.00 | 23.78 |
| ATOM | 7755 | CB | ASP | 1139 | −5.746 | −43.614 | 10.635 | 1.00 | 27.59 |
| ATOM | 7756 | CG | ASP | 1139 | −5.008 | −42.595 | 9.841 | 1.00 | 30.02 |
| ATOM | 7757 | OD1 | ASP | 1139 | −5.661 | −41.880 | 9.048 | 1.00 | 26.93 |
| ATOM | 7758 | OD2 | ASP | 1139 | −3.776 | −42.508 | 10.021 | 1.00 | 33.99 |
| ATOM | 7759 | C | ASP | 1139 | −6.398 | −41.781 | 12.260 | 1.00 | 19.81 |
| ATOM | 7760 | O | ASP | 1139 | −7.592 | −41.536 | 12.137 | 1.00 | 17.74 |
| ATOM | 7761 | N | ILE | 1140 | −5.505 | −40.832 | 12.548 | 1.00 | 18.99 |
| ATOM | 7762 | CA | ILE | 1140 | −5.937 | −39.445 | 12.737 | 1.00 | 16.56 |
| ATOM | 7763 | CB | ILE | 1140 | −4.777 | −38.540 | 13.260 | 1.00 | 15.40 |
| ATOM | 7764 | CG2 | ILE | 1140 | −5.216 | −37.074 | 13.303 | 1.00 | 14.63 |
| ATOM | 7765 | CG1 | ILE | 1140 | −4.378 | −38.987 | 14.673 | 1.00 | 14.66 |
| ATOM | 7766 | CD1 | ILE | 1140 | −3.217 | −38.193 | 15.276 | 1.00 | 14.86 |
| ATOM | 7767 | C | ILE | 1140 | −6.576 | −38.792 | 11.507 | 1.00 | 15.51 |
| ATOM | 7768 | O | ILE | 1140 | −7.594 | −38.099 | 11.627 | 1.00 | 15.94 |
| ATOM | 7769 | N | ARG | 1141 | −6.006 | −39.003 | 10.325 | 1.00 | 15.97 |
| ATOM | 7770 | CA | ARG | 1141 | −6.596 | −38.411 | 9.129 | 1.00 | 17.73 |
| ATOM | 7771 | CB | ARG | 1141 | −5.678 | −38.608 | 7.916 | 1.00 | 18.46 |
| ATOM | 7772 | CG | ARG | 1141 | −4.473 | −37.682 | 7.944 | 1.00 | 20.14 |
| ATOM | 7773 | CD | ARG | 1141 | −3.493 | −37.933 | 6.816 | 1.00 | 19.99 |
| ATOM | 7774 | NE | ARG | 1141 | −2.379 | −36.984 | 6.875 | 1.00 | 21.20 |
| ATOM | 7775 | CZ | ARG | 1141 | −1.465 | −36.945 | 7.843 | 1.00 | 21.99 |
| ATOM | 7776 | NH1 | ARG | 1141 | −1.511 | −37.805 | 8.851 | 1.00 | 20.45 |
| ATOM | 7777 | NH2 | ARG | 1141 | −0.497 | −36.042 | 7.804 | 1.00 | 23.21 |
| ATOM | 7778 | C | ARG | 1141 | −7.970 | −39.026 | 8.885 | 1.00 | 17.66 |
| ATOM | 7779 | O | ARG | 1141 | −8.910 | −38.330 | 8.484 | 1.00 | 16.58 |
| ATOM | 7780 | N | ALA | 1142 | −8.096 | −40.325 | 9.146 | 1.00 | 17.72 |
| ATOM | 7781 | CA | ALA | 1142 | −9.377 | −41.004 | 8.963 | 1.00 | 17.43 |
| ATOM | 7782 | CB | ALA | 1142 | −9.208 | −42.515 | 9.140 | 1.00 | 19.12 |
| ATOM | 7783 | C | ALA | 1142 | −10.395 | −40.471 | 9.962 | 1.00 | 19.39 |
| ATOM | 7784 | O | ALA | 1142 | −11.594 | −40.372 | 9.663 | 1.00 | 18.01 |
| ATOM | 7785 | N | ALA | 1143 | −9.925 | −40.132 | 11.158 | 1.00 | 17.42 |
| ATOM | 7786 | CA | ALA | 1143 | −10.824 | −39.600 | 12.177 | 1.00 | 18.14 |
| ATOM | 7787 | CB | ALA | 1143 | −10.087 | −39.468 | 13.510 | 1.00 | 17.85 |
| ATOM | 7788 | C | ALA | 1143 | −11.350 | −38.240 | 11.716 | 1.00 | 16.90 |
| ATOM | 7789 | O | ALA | 1143 | −12.525 | −37.921 | 11.900 | 1.00 | 16.07 |
| ATOM | 7790 | N | VAL | 1144 | −10.464 | −37.449 | 11.115 | 1.00 | 16.80 |
| ATOM | 7791 | CA | VAL | 1144 | −10.826 | −36.135 | 10.603 | 1.00 | 17.64 |
| ATOM | 7792 | CB | VAL | 1144 | −9.604 | −35.412 | 9.989 | 1.00 | 17.57 |
| ATOM | 7793 | CG1 | VAL | 1144 | −10.050 | −34.114 | 9.326 | 1.00 | 15.63 |
| ATOM | 7794 | CG2 | VAL | 1144 | −8.561 | −35.116 | 11.078 | 1.00 | 15.39 |
| ATOM | 7795 | C | VAL | 1144 | −11.900 | −36.290 | 9.528 | 1.00 | 18.77 |
| ATOM | 7796 | O | VAL | 1144 | −12.928 | −35.615 | 9.560 | 1.00 | 17.83 |
| ATOM | 7797 | N | ARG | 1145 | −11.656 | −37.181 | 8.575 | 1.00 | 19.43 |
| ATOM | 7798 | CA | ARG | 1145 | −12.619 | −37.408 | 7.507 | 1.00 | 21.72 |
| ATOM | 7799 | CB | ARG | 1145 | −12.079 | −38.435 | 6.514 | 1.00 | 22.86 |
| ATOM | 7800 | CG | ARG | 1145 | −10.907 | −37.920 | 5.690 | 1.00 | 23.87 |
| ATOM | 7801 | CD | ARG | 1145 | −10.570 | −38.881 | 4.565 | 1.00 | 26.94 |
| ATOM | 7802 | NE | ARG | 1145 | −10.060 | −40.162 | 5.055 | 1.00 | 27.22 |
| ATOM | 7803 | CZ | ARG | 1145 | −8.771 | −40.448 | 5.207 | 1.00 | 26.31 |
| ATOM | 7804 | NH1 | ARG | 1145 | −7.846 | −39.544 | 4.909 | 1.00 | 25.59 |
| ATOM | 7805 | NH2 | ARG | 1145 | −8.407 | −41.646 | 5.639 | 1.00 | 27.26 |
| ATOM | 7806 | C | ARG | 1145 | −13.965 | −37.865 | 8.050 | 1.00 | 22.32 |
| ATOM | 7807 | O | ARG | 1145 | −15.010 | −37.470 | 7.531 | 1.00 | 22.86 |
| ATOM | 7808 | N | GLN | 1146 | −13.941 | −38.685 | 9.098 | 1.00 | 21.32 |
| ATOM | 7809 | CA | GLN | 1146 | −15.171 | −39.190 | 9.701 | 1.00 | 22.69 |
| ATOM | 7810 | CB | GLN | 1146 | −14.855 | −40.318 | 10.691 | 1.00 | 24.27 |
| ATOM | 7811 | CG | GLN | 1146 | −16.081 | −41.095 | 11.161 | 1.00 | 29.43 |
| ATOM | 7812 | CD | GLN | 1146 | −15.732 | −42.213 | 12.132 | 1.00 | 34.61 |
| ATOM | 7813 | OE1 | GLN | 1146 | −14.892 | −43.067 | 11.839 | 1.00 | 36.88 |
| ATOM | 7814 | NE2 | GLN | 1146 | −16.382 | −42.216 | 13.293 | 1.00 | 35.93 |
| ATOM | 7815 | C | GLN | 1146 | −15.918 | −38.077 | 10.423 | 1.00 | 22.56 |
| ATOM | 7816 | O | GLN | 1146 | −17.152 | −38.048 | 10.448 | 1.00 | 19.62 |
| ATOM | 7817 | N | TYR | 1147 | −15.167 | −37.165 | 11.027 | 1.00 | 20.38 |
| ATOM | 7818 | CA | TYR | 1147 | −15.774 | −36.051 | 11.739 | 1.00 | 22.67 |
| ATOM | 7819 | CB | TYR | 1147 | −14.690 | −35.256 | 12.478 | 1.00 | 20.90 |
| ATOM | 7820 | CG | TYR | 1147 | −15.136 | −33.900 | 12.979 | 1.00 | 21.86 |
| ATOM | 7821 | CD1 | TYR | 1147 | −16.312 | −33.756 | 13.721 | 1.00 | 20.32 |
| ATOM | 7822 | CE1 | TYR | 1147 | −16.718 | −32.511 | 14.186 | 1.00 | 21.50 |
| ATOM | 7823 | CD2 | TYR | 1147 | −14.374 | −32.759 | 12.719 | 1.00 | 20.91 |
| ATOM | 7824 | CE2 | TYR | 1147 | −14.769 | −31.509 | 13.180 | 1.00 | 20.76 |
| ATOM | 7825 | CZ | TYR | 1147 | −15.939 | −31.389 | 13.910 | 1.00 | 22.56 |
| ATOM | 7826 | OH | TYR | 1147 | −16.345 | −30.152 | 14.358 | 1.00 | 21.72 |
| ATOM | 7827 | C | TYR | 1147 | −16.509 | −35.161 | 10.737 | 1.00 | 22.11 |
| ATOM | 7828 | O | TYR | 1147 | −17.643 | −34.752 | 10.969 | 1.00 | 23.56 |
| ATOM | 7829 | N | MET | 1148 | −15.855 | −34.881 | 9.618 | 1.00 | 22.47 |
| ATOM | 7830 | CA | MET | 1148 | −16.441 | −34.051 | 8.580 | 1.00 | 23.31 |
| ATOM | 7831 | CB | MET | 1148 | −15.427 | −33.835 | 7.459 | 1.00 | 24.14 |
| ATOM | 7832 | CG | MET | 1148 | −14.182 | −33.075 | 7.879 | 1.00 | 25.93 |
| ATOM | 7833 | SD | MET | 1148 | −12.910 | −33.130 | 6.619 | 1.00 | 28.97 |
| ATOM | 7834 | CE | MET | 1148 | −13.585 | −32.034 | 5.386 | 1.00 | 28.81 |
| ATOM | 7835 | C | MET | 1148 | −17.701 | −34.704 | 8.012 | 1.00 | 25.12 |
| ATOM | 7836 | O | MET | 1148 | −18.743 | −34.055 | 7.868 | 1.00 | 24.49 |
| ATOM | 7837 | N | ALA | 1149 | −17.599 | −35.992 | 7.700 | 1.00 | 24.17 |
| ATOM | 7838 | CA | ALA | 1149 | −18.718 | −36.747 | 7.133 | 1.00 | 24.28 |
| ATOM | 7839 | CB | ALA | 1149 | −18.254 | −38.152 | 6.750 | 1.00 | 23.46 |
| ATOM | 7840 | C | ALA | 1149 | −19.914 | −36.828 | 8.079 | 1.00 | 24.06 |
| ATOM | 7841 | O | ALA | 1149 | −21.058 | −36.613 | 7.669 | 1.00 | 23.40 |
| ATOM | 7842 | N | GLU | 1150 | −19.657 | −37.135 | 9.346 | 1.00 | 24.16 |
| ATOM | 7843 | CA | GLU | 1150 | −20.739 | −37.235 | 10.319 | 1.00 | 26.28 |
| ATOM | 7844 | CB | GLU | 1150 | −20.228 | −37.828 | 11.634 | 1.00 | 27.57 |
| ATOM | 7845 | CG | GLU | 1150 | −20.156 | −39.347 | 11.608 | 1.00 | 28.54 |
| ATOM | 7846 | CD | GLU | 1150 | −19.614 | −39.939 | 12.890 | 1.00 | 28.17 |
| ATOM | 7847 | OE1 | GLU | 1150 | −20.027 | −39.506 | 13.984 | 1.00 | 28.42 |
| ATOM | 7848 | OE2 | GLU | 1150 | −18.778 | −40.857 | 12.796 | 1.00 | 33.92 |
| ATOM | 7849 | C | GLU | 1150 | −21.421 | −35.900 | 10.581 | 1.00 | 26.66 |
| ATOM | 7850 | O | GLU | 1150 | −22.597 | −35.859 | 10.949 | 1.00 | 27.42 |
| ATOM | 7851 | N | VAL | 1151 | −20.685 | −34.808 | 10.408 | 1.00 | 27.27 |
| ATOM | 7852 | CA | VAL | 1151 | −21.263 | −33.486 | 10.617 | 1.00 | 27.27 |
| ATOM | 7853 | CB | VAL | 1151 | −20.179 | −32.384 | 10.668 | 1.00 | 27.12 |
| ATOM | 7854 | CG1 | VAL | 1151 | −20.835 | −31.005 | 10.609 | 1.00 | 23.63 |
| ATOM | 7855 | CG2 | VAL | 1151 | −19.372 | −32.512 | 11.945 | 1.00 | 23.64 |
| ATOM | 7856 | C | VAL | 1151 | −22.242 | −33.154 | 9.495 | 1.00 | 28.77 |
| ATOM | 7857 | O | VAL | 1151 | −23.384 | −32.777 | 9.749 | 1.00 | 28.71 |
| ATOM | 7858 | N | GLU | 1152 | −21.792 | −33.304 | 8.256 | 1.00 | 30.65 |
| ATOM | 7859 | CA | GLU | 1152 | −22.637 | −32.998 | 7.112 | 1.00 | 33.92 |
| ATOM | 7860 | CB | GLU | 1152 | −21.836 | −33.117 | 5.813 | 1.00 | 34.31 |
| ATOM | 7861 | CG | GLU | 1152 | −22.641 | −32.774 | 4.567 | 1.00 | 38.13 |
| ATOM | 7862 | CD | GLU | 1152 | −21.775 | −32.545 | 3.345 | 1.00 | 39.92 |
| ATOM | 7863 | OE1 | GLU | 1152 | −22.337 | −32.401 | 2.240 | 1.00 | 43.20 |
| ATOM | 7864 | OE2 | GLU | 1152 | −20.535 | −32.498 | 3.485 | 1.00 | 41.00 |
| ATOM | 7865 | C | GLU | 1152 | −23.867 | −33.895 | 7.051 | 1.00 | 34.76 |
| ATOM | 7866 | O | GLU | 1152 | −24.942 | −33.456 | 6.640 | 1.00 | 35.70 |
| ATOM | 7867 | N | SER | 1153 | −23.709 | −35.147 | 7.468 | 1.00 | 34.67 |
| ATOM | 7868 | CA | SER | 1153 | −24.814 | −36.102 | 7.456 | 1.00 | 34.62 |
| ATOM | 7869 | CB | SER | 1153 | −24.284 | −37.534 | 7.559 | 1.00 | 35.72 |
| ATOM | 7870 | OG | SER | 1153 | −23.645 | −37.926 | 6.357 | 1.00 | 39.86 |
| ATOM | 7871 | C | SER | 1153 | −25.787 | −35.852 | 8.596 | 1.00 | 32.97 |
| ATOM | 7872 | O | SER | 1153 | −26.974 | −36.157 | 8.487 | 1.00 | 33.54 |
| ATOM | 7873 | N | GLY | 1154 | −25.280 | −35.302 | 9.692 | 1.00 | 30.02 |
| ATOM | 7874 | CA | GLY | 1154 | −26.130 | −35.039 | 10.835 | 1.00 | 28.64 |
| ATOM | 7875 | C | GLY | 1154 | −25.952 | −36.090 | 11.912 | 1.00 | 27.31 |
| ATOM | 7876 | O | GLY | 1154 | −26.491 | −35.960 | 13.008 | 1.00 | 27.96 |
| ATOM | 7877 | N | VAL | 1155 | −25.197 | −37.140 | 11.599 | 1.00 | 27.21 |
| ATOM | 7878 | CA | VAL | 1155 | −24.944 | −38.208 | 12.558 | 1.00 | 27.30 |
| ATOM | 7879 | CB | VAL | 1155 | −23.969 | −39.252 | 11.984 | 1.00 | 27.92 |
| ATOM | 7880 | CG1 | VAL | 1155 | −23.728 | −40.357 | 13.005 | 1.00 | 30.23 |
| ATOM | 7881 | CG2 | VAL | 1155 | −24.528 | −39.828 | 10.692 | 1.00 | 31.88 |
| ATOM | 7882 | C | VAL | 1155 | −24.333 | −37.614 | 13.823 | 1.00 | 26.47 |
| ATOM | 7883 | O | VAL | 1155 | −24.616 | −38.065 | 14.935 | 1.00 | 26.28 |
| ATOM | 7884 | N | TYR | 1156 | −23.485 | −36.606 | 13.636 | 1.00 | 25.70 |
| ATOM | 7885 | CA | TYR | 1156 | −22.828 | −35.921 | 14.746 | 1.00 | 25.82 |
| ATOM | 7886 | CB | TYR | 1156 | −21.307 | −36.124 | 14.709 | 1.00 | 24.74 |
| ATOM | 7887 | CG | TYR | 1156 | −20.605 | −35.437 | 15.867 | 1.00 | 24.07 |
| ATOM | 7888 | CD1 | TYR | 1156 | −20.649 | −35.976 | 17.156 | 1.00 | 23.28 |
| ATOM | 7889 | CE1 | TYR | 1156 | −20.078 | −35.306 | 18.242 | 1.00 | 23.96 |
| ATOM | 7890 | CD2 | TYR | 1156 | −19.964 | −34.213 | 15.690 | 1.00 | 24.41 |
| ATOM | 7891 | CE2 | TYR | 1156 | −19.392 | −33.533 | 16.768 | 1.00 | 24.50 |
| ATOM | 7892 | CZ | TYR | 1156 | −19.455 | −34.083 | 18.041 | 1.00 | 24.23 |
| ATOM | 7893 | OH | TYR | 1156 | −18.920 | −33.395 | 19.112 | 1.00 | 23.15 |
| ATOM | 7894 | C | TYR | 1156 | −23.116 | −34.424 | 14.696 | 1.00 | 26.27 |
| ATOM | 7895 | O | TYR | 1156 | −22.996 | −33.799 | 13.642 | 1.00 | 26.36 |
| ATOM | 7896 | N | PRO | 1157 | −23.471 | −33.826 | 15.844 | 1.00 | 27.37 |
| ATOM | 7897 | CD | PRO | 1157 | −23.555 | −32.365 | 16.017 | 1.00 | 28.85 |
| ATOM | 7898 | CA | PRO | 1157 | −23.600 | −34.509 | 17.132 | 1.00 | 28.73 |
| ATOM | 7899 | CB | PRO | 1157 | −23.499 | −33.365 | 18.138 | 1.00 | 28.98 |
| ATOM | 7900 | CG | PRO | 1157 | −24.117 | −32.234 | 17.416 | 1.00 | 29.31 |
| ATOM | 7901 | C | PRO | 1157 | −24.882 | −35.319 | 17.290 | 1.00 | 30.00 |
| ATOM | 7902 | O | PRO | 1157 | −25.928 | −34.970 | 16.740 | 1.00 | 29.72 |
| ATOM | 7903 | N | GLY | 1158 | −24.786 | −36.409 | 18.046 | 1.00 | 30.34 |
| ATOM | 7904 | CA | GLY | 1158 | −25.940 | −37.257 | 18.278 | 1.00 | 31.69 |
| ATOM | 7905 | C | GLY | 1158 | −26.668 | −36.843 | 19.540 | 1.00 | 32.83 |
| ATOM | 7906 | O | GLY | 1158 | −26.240 | −35.921 | 20.234 | 1.00 | 31.48 |
| ATOM | 7907 | N | GLU | 1159 | −27.770 | −37.521 | 19.844 | 1.00 | 33.48 |
| ATOM | 7908 | CA | GLU | 1159 | −28.538 | −37.198 | 21.036 | 1.00 | 34.89 |
| ATOM | 7909 | CB | GLU | 1159 | −29.791 | −38.075 | 21.122 | 1.00 | 36.68 |
| ATOM | 7910 | CG | GLU | 1159 | −30.853 | −37.515 | 22.049 | 1.00 | 38.05 |
| ATOM | 7911 | CD | GLU | 1159 | −31.472 | −36.241 | 21.503 | 1.00 | 39.50 |
| ATOM | 7912 | OE1 | GLU | 1159 | −32.152 | −35.527 | 22.269 | 1.00 | 41.54 |
| ATOM | 7913 | OE2 | GLU | 1159 | −31.282 | −35.958 | 20.300 | 1.00 | 39.55 |
| ATOM | 7914 | C | GLU | 1159 | −27.685 | −37.403 | 22.283 | 1.00 | 35.20 |
| ATOM | 7915 | O | GLU | 1159 | −27.943 | −36.802 | 23.325 | 1.00 | 35.87 |
| ATOM | 7916 | N | GLU | 1160 | −26.668 | −38.256 | 22.167 | 1.00 | 35.14 |
| ATOM | 7917 | CA | GLU | 1160 | −25.764 | −38.545 | 23.277 | 1.00 | 35.10 |
| ATOM | 7918 | CB | GLU | 1160 | −24.870 | −39.748 | 22.951 | 1.00 | 37.43 |
| ATOM | 7919 | CG | GLU | 1160 | −25.577 | −40.902 | 22.261 | 1.00 | 41.76 |
| ATOM | 7920 | CD | GLU | 1160 | −25.822 | −40.638 | 20.789 | 1.00 | 42.93 |
| ATOM | 7921 | OE1 | GLU | 1160 | −24.834 | −40.410 | 20.054 | 1.00 | 43.83 |
| ATOM | 7922 | OE2 | GLU | 1160 | −26.999 | −40.666 | 20.368 | 1.00 | 43.38 |
| ATOM | 7923 | C | GLU | 1160 | −24.862 | −37.353 | 23.583 | 1.00 | 33.30 |
| ATOM | 7924 | O | GLU | 1160 | −24.259 | −37.285 | 24.654 | 1.00 | 31.07 |
| ATOM | 7925 | N | HIS | 1161 | −24.777 | −36.422 | 22.637 | 1.00 | 32.51 |
| ATOM | 7926 | CA | HIS | 1161 | −23.930 | −35.238 | 22.781 | 1.00 | 32.09 |
| ATOM | 7927 | CB | HIS | 1161 | −23.016 | −35.097 | 21.562 | 1.00 | 31.02 |
| ATOM | 7928 | CG | HIS | 1161 | −22.355 | −36.373 | 21.143 | 1.00 | 29.12 |
| ATOM | 7929 | CD2 | HIS | 1161 | −22.512 | −37.131 | 20.032 | 1.00 | 27.73 |
| ATOM | 7930 | ND1 | HIS | 1161 | −21.407 | −37.013 | 21.913 | 1.00 | 28.00 |
| ATOM | 7931 | CE1 | HIS | 1161 | −21.012 | −38.112 | 21.296 | 1.00 | 27.95 |
| ATOM | 7932 | NE2 | HIS | 1161 | −21.667 | −38.207 | 20.152 | 1.00 | 29.03 |
| ATOM | 7933 | C | HIS | 1161 | −24.757 | −33.965 | 22.899 | 1.00 | 33.71 |
| ATOM | 7934 | O | HIS | 1161 | −24.212 | −32.864 | 22.874 | 1.00 | 31.48 |
| ATOM | 7935 | N | SER | 1162 | −26.069 | −34.121 | 23.028 | 1.00 | 34.90 |
| ATOM | 7936 | CA | SER | 1162 | −26.969 | −32.981 | 23.107 | 1.00 | 36.32 |
| ATOM | 7937 | CB | SER | 1162 | −28.088 | −33.167 | 22.088 | 1.00 | 36.36 |
| ATOM | 7938 | OG | SER | 1162 | −27.543 | −33.422 | 20.806 | 1.00 | 37.50 |
| ATOM | 7939 | C | SER | 1162 | −27.562 | −32.748 | 24.492 | 1.00 | 37.54 |
| ATOM | 7940 | O | SER | 1162 | −27.649 | −33.670 | 25.303 | 1.00 | 37.53 |
| ATOM | 7941 | N | PHE | 1163 | −27.969 | −31.505 | 24.749 | 1.00 | 38.51 |
| ATOM | 7942 | CA | PHE | 1163 | −28.563 | −31.129 | 26.028 | 1.00 | 39.57 |
| ATOM | 7943 | CB | PHE | 1163 | −27.764 | −30.000 | 26.692 | 1.00 | 40.57 |
| ATOM | 7944 | CG | PHE | 1163 | −26.323 | −30.346 | 26.967 | 1.00 | 43.95 |
| ATOM | 7945 | CD1 | PHE | 1163 | −25.363 | −30.253 | 25.961 | 1.00 | 44.75 |
| ATOM | 7946 | CD2 | PHE | 1163 | −25.926 | −30.771 | 28.232 | 1.00 | 44.14 |
| ATOM | 7947 | CE1 | PHE | 1163 | −24.029 | −30.575 | 26.213 | 1.00 | 45.36 |
| ATOM | 7948 | CE2 | PHE | 1163 | −24.595 | −31.096 | 28.492 | 1.00 | 45.59 |
| ATOM | 7949 | CZ | PHE | 1163 | −23.645 | −30.998 | 27.480 | 1.00 | 44.94 |
| ATOM | 7950 | C | PHE | 1163 | −30.007 | −30.676 | 25.838 | 1.00 | 39.51 |
| ATOM | 7951 | O | PHE | 1163 | −30.449 | −30.433 | 24.715 | 1.00 | 39.54 |
| ATOM | 7952 | N | HIS | 1164 | −30.736 | −30.562 | 26.944 | 1.00 | 40.34 |
| ATOM | 7953 | CA | HIS | 1164 | −32.132 | −30.136 | 26.912 | 1.00 | 40.45 |
| ATOM | 7954 | CB | HIS | 1164 | −33.056 | −31.351 | 26.812 | 1.00 | 39.13 |
| ATOM | 7955 | CG | HIS | 1164 | −32.963 | −32.071 | 25.504 | 1.00 | 37.46 |
| ATOM | 7956 | CD2 | HIS | 1164 | −32.563 | −33.329 | 25.201 | 1.00 | 36.29 |
| ATOM | 7957 | ND1 | HIS | 1164 | −33.293 | −31.476 | 24.305 | 1.00 | 36.11 |
| ATOM | 7958 | CE1 | HIS | 1164 | −33.100 | −32.336 | 23.321 | 1.00 | 34.87 |
| ATOM | 7959 | NE2 | HIS | 1164 | −32.657 | −33.468 | 23.837 | 1.00 | 34.05 |
| ATOM | 7960 | C | HIS | 1164 | −32.484 | −29.320 | 28.149 | 1.00 | 41.45 |
| ATOM | 7961 | O | HIS | 1164 | −33.130 | −28.261 | 27.988 | 1.00 | 42.79 |
| ATOM | 7962 | OXT | HIS | 1164 | −32.118 | −29.749 | 29.264 | 1.00 | 42.44 |
| ATOM | 7963 | C1 | KPL | 1165 | −14.350 | −24.823 | 22.600 | 1.00 | 37.46 |
| ATOM | 7964 | C2 | KPL | 1165 | −14.556 | −23.614 | 21.664 | 1.00 | 35.76 |
| ATOM | 7965 | C3 | KPL | 1165 | −14.275 | −24.044 | 20.219 | 1.00 | 36.86 |
| ATOM | 7966 | C4 | KPL | 1165 | −16.018 | −23.142 | 21.763 | 1.00 | 37.77 |
| ATOM | 7967 | O1 | KPL | 1165 | −16.308 | −22.744 | 23.112 | 1.00 | 39.26 |
| ATOM | 7968 | C5 | KPL | 1165 | −13.590 | −22.469 | 22.059 | 1.00 | 34.45 |
| ATOM | 7969 | O2 | KPL | 1165 | −14.027 | −21.380 | 22.384 | 1.00 | 34.78 |
| ATOM | 7970 | C6 | KPL | 1165 | −12.093 | −22.661 | 22.058 | 1.00 | 32.52 |
| ATOM | 7971 | O3 | KPL | 1165 | −11.604 | −23.720 | 21.736 | 1.00 | 31.95 |
| ATOM | 7972 | O4 | KPL | 1165 | −11.288 | −21.651 | 22.419 | 1.00 | 28.43 |
| ATOM | 7973 | CB | MET | 1201 | −15.474 | −28.638 | 47.750 | 1.00 | 74.61 |
| ATOM | 7974 | CG | MET | 1201 | −16.080 | −29.937 | 48.288 | 1.00 | 76.04 |
| ATOM | 7975 | SD | MET | 1201 | −15.715 | −31.415 | 47.313 | 1.00 | 77.75 |
| ATOM | 7976 | CE | MET | 1201 | −17.296 | −31.664 | 46.480 | 1.00 | 77.07 |
| ATOM | 7977 | C | MET | 1201 | −15.060 | −28.920 | 45.290 | 1.00 | 71.84 |
| ATOM | 7978 | O | MET | 1201 | −13.988 | −29.442 | 45.601 | 1.00 | 72.20 |
| ATOM | 7979 | N | MET | 1201 | −17.380 | −28.524 | 46.164 | 1.00 | 73.50 |
| ATOM | 7980 | CA | MET | 1201 | −15.929 | −28.231 | 46.340 | 1.00 | 73.03 |
| ATOM | 7981 | N | LYS | 1202 | −15.525 | −28.905 | 44.043 | 1.00 | 69.78 |
| ATOM | 7982 | CA | LYS | 1202 | −14.795 | −29.524 | 42.940 | 1.00 | 67.53 |
| ATOM | 7983 | CB | LYS | 1202 | −15.523 | −30.791 | 42.469 | 1.00 | 68.57 |
| ATOM | 7984 | CG | LYS | 1202 | −15.422 | −31.977 | 43.422 | 1.00 | 69.98 |
| ATOM | 7985 | CD | LYS | 1202 | −14.031 | −32.611 | 43.409 | 1.00 | 70.39 |
| ATOM | 7986 | CE | LYS | 1202 | −13.781 | −33.424 | 42.140 | 1.00 | 70.57 |
| ATOM | 7987 | NZ | LYS | 1202 | −13.778 | −32.606 | 40.895 | 1.00 | 70.61 |
| ATOM | 7988 | C | LYS | 1202 | −14.609 | −28.580 | 41.752 | 1.00 | 64.73 |
| ATOM | 7989 | O | LYS | 1202 | −15.029 | −28.890 | 40.637 | 1.00 | 65.32 |
| ATOM | 7990 | N | PRO | 1203 | −13.973 | −27.414 | 41.973 | 1.00 | 61.55 |
| ATOM | 7991 | CD | PRO | 1203 | −13.472 | −26.591 | 40.856 | 1.00 | 60.49 |
| ATOM | 7992 | CA | PRO | 1203 | −13.433 | −26.929 | 43.248 | 1.00 | 58.33 |
| ATOM | 7993 | CB | PRO | 1203 | −12.232 | −26.108 | 42.802 | 1.00 | 58.95 |
| ATOM | 7994 | CG | PRO | 1203 | −12.755 | −25.453 | 41.571 | 1.00 | 59.90 |
| ATOM | 7995 | C | PRO | 1203 | −14.454 | −26.086 | 44.018 | 1.00 | 55.17 |
| ATOM | 7996 | O | PRO | 1203 | −15.641 | −26.081 | 43.689 | 1.00 | 55.08 |
| ATOM | 7997 | N | THR | 1204 | −13.987 | −25.372 | 45.039 | 1.00 | 50.88 |
| ATOM | 7998 | CA | THR | 1204 | −14.863 | −24.522 | 45.843 | 1.00 | 46.61 |
| ATOM | 7999 | CB | THR | 1204 | −14.165 | −24.061 | 47.140 | 1.00 | 46.19 |
| ATOM | 8000 | OG1 | THR | 1204 | −13.702 | −25.204 | 47.870 | 1.00 | 46.22 |
| ATOM | 8001 | CG2 | THR | 1204 | −15.131 | −23.273 | 48.009 | 1.00 | 45.19 |
| ATOM | 8002 | C | THR | 1204 | −15.240 | −23.288 | 45.026 | 1.00 | 43.63 |
| ATOM | 8003 | O | THR | 1204 | −14.373 | −22.642 | 44.440 | 1.00 | 42.49 |
| ATOM | 8004 | N | THR | 1205 | −16.531 | −22.966 | 44.985 | 1.00 | 40.26 |
| ATOM | 8005 | CA | THR | 1205 | −17.000 | −21.811 | 44.222 | 1.00 | 37.82 |
| ATOM | 8006 | CB | THR | 1205 | −17.760 | −22.241 | 42.953 | 1.00 | 38.23 |
| ATOM | 8007 | OG1 | THR | 1205 | −18.955 | −22.937 | 43.330 | 1.00 | 39.06 |
| ATOM | 8008 | CG2 | THR | 1205 | −16.893 | −23.142 | 42.089 | 1.00 | 39.20 |
| ATOM | 8009 | C | THR | 1205 | −17.924 | −20.887 | 45.002 | 1.00 | 35.47 |
| ATOM | 8010 | O | THR | 1205 | −18.360 | −21.201 | 46.111 | 1.00 | 34.25 |
| ATOM | 8011 | N | ILE | 1206 | −18.226 | −19.744 | 44.397 | 1.00 | 33.47 |
| ATOM | 8012 | CA | ILE | 1206 | −19.104 | −18.761 | 45.010 | 1.00 | 34.02 |
| ATOM | 8013 | CB | ILE | 1206 | −19.263 | −17.528 | 44.107 | 1.00 | 34.29 |
| ATOM | 8014 | CG2 | ILE | 1206 | −20.055 | −16.455 | 44.834 | 1.00 | 33.15 |
| ATOM | 8015 | CG1 | ILE | 1206 | −17.880 | −16.986 | 43.728 | 1.00 | 36.56 |
| ATOM | 8016 | CD1 | ILE | 1206 | −17.908 | −15.950 | 42.616 | 1.00 | 36.92 |
| ATOM | 8017 | C | ILE | 1206 | −20.481 | −19.368 | 45.266 | 1.00 | 33.47 |
| ATOM | 8018 | O | ILE | 1206 | −21.119 | −19.080 | 46.280 | 1.00 | 33.95 |
| ATOM | 8019 | N | SER | 1207 | −20.933 | −20.218 | 44.350 | 1.00 | 33.47 |
| ATOM | 8020 | CA | SER | 1207 | −22.236 | −20.861 | 44.494 | 1.00 | 34.07 |
| ATOM | 8021 | CB | SER | 1207 | −22.515 | −21.766 | 43.292 | 1.00 | 34.94 |
| ATOM | 8022 | OG | SER | 1207 | −22.693 | −21.001 | 42.111 | 1.00 | 37.78 |
| ATOM | 8023 | C | SER | 1207 | −22.310 | −21.678 | 45.777 | 1.00 | 33.33 |
| ATOM | 8024 | O | SER | 1207 | −23.392 | −21.899 | 46.319 | 1.00 | 34.33 |
| ATOM | 8025 | N | LEU | 1208 | −21.152 | −22.121 | 46.259 | 1.00 | 32.96 |
| ATOM | 8026 | CA | LEU | 1208 | −21.086 | −22.919 | 47.477 | 1.00 | 32.87 |
| ATOM | 8027 | CB | LEU | 1208 | −19.676 | −23.496 | 47.651 | 1.00 | 34.16 |
| ATOM | 8028 | CG | LEU | 1208 | −19.539 | −24.923 | 48.189 | 1.00 | 35.32 |
| ATOM | 8029 | CD1 | LEU | 1208 | −18.071 | −25.215 | 48.489 | 1.00 | 34.07 |
| ATOM | 8030 | CD2 | LEU | 1208 | −20.368 | −25.092 | 49.440 | 1.00 | 34.02 |
| ATOM | 8031 | C | LEU | 1208 | −21.444 | −22.062 | 48.687 | 1.00 | 31.67 |
| ATOM | 8032 | O | LEU | 1208 | −22.198 | −22.488 | 49.563 | 1.00 | 30.68 |
| ATOM | 8033 | N | LEU | 1209 | −20.902 | −20.848 | 48.728 | 1.00 | 29.45 |
| ATOM | 8034 | CA | LEU | 1209 | −21.158 | −19.934 | 49.834 | 1.00 | 28.92 |
| ATOM | 8035 | CB | LEU | 1209 | −20.185 | −18.753 | 49.772 | 1.00 | 25.98 |
| ATOM | 8036 | CG | LEU | 1209 | −18.698 | −19.117 | 49.742 | 1.00 | 25.45 |
| ATOM | 8037 | CD1 | LEU | 1209 | −17.859 | −17.844 | 49.746 | 1.00 | 22.70 |
| ATOM | 8038 | CD2 | LEU | 1209 | −18.356 | −19.975 | 50.947 | 1.00 | 23.49 |
| ATOM | 8039 | C | LEU | 1209 | −22.595 | −19.425 | 49.802 | 1.00 | 29.02 |
| ATOM | 8040 | O | LEU | 1209 | −23.207 | −19.198 | 50.844 | 1.00 | 28.22 |
| ATOM | 8041 | N | GLN | 1210 | −23.126 | −19.239 | 48.598 | 1.00 | 30.69 |
| ATOM | 8042 | CA | GLN | 1210 | −24.497 | −18.763 | 48.436 | 1.00 | 32.68 |
| ATOM | 8043 | CB | GLN | 1210 | −24.777 | −18.499 | 46.954 | 1.00 | 32.40 |
| ATOM | 8044 | CG | GLN | 1210 | −26.138 | −17.885 | 46.648 | 1.00 | 33.66 |
| ATOM | 8045 | CD | GLN | 1210 | −26.378 | −16.573 | 47.372 | 1.00 | 33.08 |
| ATOM | 8046 | OE1 | GLN | 1210 | −26.857 | −16.553 | 48.506 | 1.00 | 33.39 |
| ATOM | 8047 | NE2 | GLN | 1210 | −26.037 | −15.467 | 46.722 | 1.00 | 31.00 |
| ATOM | 8048 | C | GLN | 1210 | −25.436 | −19.831 | 48.994 | 1.00 | 33.63 |
| ATOM | 8049 | O | GLN | 1210 | −26.437 | −19.520 | 49.643 | 1.00 | 34.19 |
| ATOM | 8050 | N | LYS | 1211 | −25.095 | −21.093 | 48.750 | 1.00 | 35.83 |
| ATOM | 8051 | CA | LYS | 1211 | −25.888 | −22.212 | 49.247 | 1.00 | 37.75 |
| ATOM | 8052 | CB | LYS | 1211 | −25.367 | −23.533 | 48.670 | 1.00 | 39.41 |
| ATOM | 8053 | CG | LYS | 1211 | −25.864 | −24.786 | 49.413 | 1.00 | 43.61 |
| ATOM | 8054 | CD | LYS | 1211 | −25.471 | −26.064 | 48.717 | 1.00 | 46.26 |
| ATOM | 8055 | CE | LYS | 1211 | −26.318 | −26.314 | 47.477 | 1.00 | 47.80 |
| ATOM | 8056 | NZ | LYS | 1211 | −26.008 | −27.629 | 46.849 | 1.00 | 49.02 |
| ATOM | 8057 | C | LYS | 1211 | −25.833 | −22.258 | 50.775 | 1.00 | 38.19 |
| ATOM | 8058 | O | LYS | 1211 | −26.822 | −22.579 | 51.434 | 1.00 | 37.83 |
| ATOM | 8059 | N | TYR | 1212 | −24.673 | −21.930 | 51.334 | 1.00 | 37.94 |
| ATOM | 8060 | CA | TYR | 1212 | −24.493 | −21.933 | 52.781 | 1.00 | 38.24 |
| ATOM | 8061 | CB | TYR | 1212 | −23.032 | −21.643 | 53.128 | 1.00 | 39.33 |
| ATOM | 8062 | CG | TYR | 1212 | −22.101 | −22.817 | 52.936 | 1.00 | 41.48 |
| ATOM | 8063 | CD1 | TYR | 1212 | −20.724 | −22.623 | 52.830 | 1.00 | 43.21 |
| ATOM | 8064 | CE1 | TYR | 1212 | −19.854 | −23.698 | 52.690 | 1.00 | 44.20 |
| ATOM | 8065 | CD2 | TYR | 1212 | −22.587 | −24.125 | 52.897 | 1.00 | 42.82 |
| ATOM | 8066 | CE2 | TYR | 1212 | −21.724 | −25.210 | 52.759 | 1.00 | 44.18 |
| ATOM | 8067 | CZ | TYR | 1212 | −20.360 | −24.987 | 52.657 | 1.00 | 44.87 |
| ATOM | 8068 | OH | TYR | 1212 | −19.496 | −26.048 | 52.522 | 1.00 | 46.35 |
| ATOM | 8069 | C | TYR | 1212 | −25.388 | −20.922 | 53.485 | 1.00 | 37.78 |
| ATOM | 8070 | O | TYR | 1212 | −25.946 | −21.211 | 54.544 | 1.00 | 36.95 |
| ATOM | 8071 | N | LYS | 1213 | −25.514 | −19.731 | 52.906 | 1.00 | 37.50 |
| ATOM | 8072 | CA | LYS | 1213 | −26.348 | −18.695 | 53.506 | 1.00 | 37.53 |
| ATOM | 8073 | CB | LYS | 1213 | −26.256 | −17.387 | 52.707 | 1.00 | 35.24 |
| ATOM | 8074 | CG | LYS | 1213 | −27.168 | −16.292 | 53.248 | 1.00 | 31.66 |
| ATOM | 8075 | CD | LYS | 1213 | −26.732 | −14.894 | 52.831 | 1.00 | 27.83 |
| ATOM | 8076 | CE | LYS | 1213 | −27.589 | −13.850 | 53.530 | 1.00 | 25.23 |
| ATOM | 8077 | NZ | LYS | 1213 | −27.068 | −12.467 | 53.408 | 1.00 | 24.51 |
| ATOM | 8078 | C | LYS | 1213 | −27.803 | −19.146 | 53.591 | 1.00 | 39.33 |
| ATOM | 8079 | O | LYS | 1213 | −28.451 | −18.980 | 54.624 | 1.00 | 39.00 |
| ATOM | 8080 | N | GLN | 1214 | −28.312 | −19.717 | 52.505 | 1.00 | 40.82 |
| ATOM | 8081 | CA | GLN | 1214 | −29.689 | −20.190 | 52.481 | 1.00 | 43.81 |
| ATOM | 8082 | CB | GLN | 1214 | −30.060 | −20.692 | 51.084 | 1.00 | 45.23 |
| ATOM | 8083 | CG | GLN | 1214 | −30.123 | −19.591 | 50.037 | 1.00 | 48.26 |
| ATOM | 8084 | CD | GLN | 1214 | −30.583 | −20.097 | 48.684 | 1.00 | 50.37 |
| ATOM | 8085 | OE1 | GLN | 1214 | −31.663 | −20.683 | 48.561 | 1.00 | 52.18 |
| ATOM | 8086 | NE2 | GLN | 1214 | −29.767 | −19.870 | 47.656 | 1.00 | 50.33 |
| ATOM | 8087 | C | GLN | 1214 | −29.889 | −21.302 | 53.501 | 1.00 | 44.29 |
| ATOM | 8088 | O | GLN | 1214 | −30.948 | −21.406 | 54.118 | 1.00 | 44.96 |
| ATOM | 8089 | N | GLU | 1215 | −28.862 | −22.126 | 53.683 | 1.00 | 45.06 |
| ATOM | 8090 | CA | GLU | 1215 | −28.934 | −23.229 | 54.633 | 1.00 | 45.32 |
| ATOM | 8091 | CB | GLU | 1215 | −27.960 | −24.343 | 54.241 | 1.00 | 46.90 |
| ATOM | 8092 | CG | GLU | 1215 | −28.102 | −24.843 | 52.815 | 1.00 | 49.76 |
| ATOM | 8093 | CD | GLU | 1215 | −27.175 | −26.008 | 52.522 | 1.00 | 51.37 |
| ATOM | 8094 | OE1 | GLU | 1215 | −25.983 | −25.927 | 52.888 | 1.00 | 52.64 |
| ATOM | 8095 | OE2 | GLU | 1215 | −27.635 | −27.000 | 51.920 | 1.00 | 52.49 |
| ATOM | 8096 | C | GLU | 1215 | −28.597 | −22.744 | 56.036 | 1.00 | 44.97 |
| ATOM | 8097 | O | GLU | 1215 | −28.513 | −23.541 | 56.969 | 1.00 | 44.92 |
| ATOM | 8098 | N | LYS | 1216 | −28.400 | −21.437 | 56.182 | 1.00 | 44.76 |
| ATOM | 8099 | CA | LYS | 1216 | −28.069 | −20.852 | 57.479 | 1.00 | 43.95 |
| ATOM | 8100 | CB | LYS | 1216 | −29.252 | −21.001 | 58.442 | 1.00 | 45.68 |
| ATOM | 8101 | CG | LYS | 1216 | −30.481 | −20.179 | 58.071 | 1.00 | 48.07 |
| ATOM | 8102 | CD | LYS | 1216 | −30.234 | −18.687 | 58.255 | 1.00 | 49.08 |
| ATOM | 8103 | CE | LYS | 1216 | −31.475 | −17.871 | 57.914 | 1.00 | 50.43 |
| ATOM | 8104 | NZ | LYS | 1216 | −32.654 | −18.265 | 58.740 | 1.00 | 51.79 |
| ATOM | 8105 | C | LYS | 1216 | −26.829 | −21.509 | 58.085 | 1.00 | 42.80 |
| ATOM | 8106 | O | LYS | 1216 | −26.763 | −21.741 | 59.296 | 1.00 | 42.94 |
| ATOM | 8107 | N | LYS | 1217 | −25.850 | −21.813 | 57.239 | 1.00 | 40.87 |
| ATOM | 8108 | CA | LYS | 1217 | −24.613 | −22.444 | 57.693 | 1.00 | 39.09 |
| ATOM | 8109 | CB | LYS | 1217 | −24.290 | −23.667 | 56.830 | 1.00 | 39.84 |
| ATOM | 8110 | CG | LYS | 1217 | −22.929 | −24.284 | 57.143 | 1.00 | 42.69 |
| ATOM | 8111 | CD | LYS | 1217 | −22.622 | −25.474 | 56.250 | 1.00 | 45.14 |
| ATOM | 8112 | CE | LYS | 1217 | −23.542 | −26.651 | 56.546 | 1.00 | 47.62 |
| ATOM | 8113 | NZ | LYS | 1217 | −23.209 | −27.838 | 55.706 | 1.00 | 48.86 |
| ATOM | 8114 | C | LYS | 1217 | −23.439 | −21.471 | 57.646 | 1.00 | 36.74 |
| ATOM | 8115 | O | LYS | 1217 | −22.930 | −21.156 | 56.573 | 1.00 | 37.26 |
| ATOM | 8116 | N | ARG | 1218 | −23.008 | −21.003 | 58.812 | 1.00 | 33.47 |
| ATOM | 8117 | CA | ARG | 1218 | −21.892 | −20.069 | 58.890 | 1.00 | 31.52 |
| ATOM | 8118 | CB | ARG | 1218 | −21.799 | −19.498 | 60.309 | 1.00 | 32.55 |
| ATOM | 8119 | CG | ARG | 1218 | −22.894 | −18.476 | 60.603 | 1.00 | 31.60 |
| ATOM | 8120 | CD | ARG | 1218 | −22.918 | −18.029 | 62.052 | 1.00 | 33.28 |
| ATOM | 8121 | NE | ARG | 1218 | −23.699 | −18.938 | 62.890 | 1.00 | 33.49 |
| ATOM | 8122 | CZ | ARG | 1218 | −24.049 | −18.678 | 64.145 | 1.00 | 32.58 |
| ATOM | 8123 | NH1 | ARG | 1218 | −23.688 | −17.537 | 64.716 | 1.00 | 33.26 |
| ATOM | 8124 | NH2 | ARG | 1218 | −24.767 | −19.554 | 64.828 | 1.00 | 34.35 |
| ATOM | 8125 | C | ARG | 1218 | −20.578 | −20.737 | 58.476 | 1.00 | 30.31 |
| ATOM | 8126 | O | ARG | 1218 | −20.223 | −21.797 | 58.990 | 1.00 | 31.64 |
| ATOM | 8127 | N | PHE | 1219 | −19.864 | −20.112 | 57.543 | 1.00 | 28.09 |
| ATOM | 8128 | CA | PHE | 1219 | −18.607 | −20.656 | 57.038 | 1.00 | 24.32 |
| ATOM | 8129 | CB | PHE | 1219 | −18.701 | −20.816 | 55.518 | 1.00 | 23.44 |
| ATOM | 8130 | CG | PHE | 1219 | −19.005 | −19.537 | 54.794 | 1.00 | 22.44 |
| ATOM | 8131 | CD1 | PHE | 1219 | −17.975 | −18.711 | 54.343 | 1.00 | 21.27 |
| ATOM | 8132 | CD2 | PHE | 1219 | −20.321 | −19.131 | 54.606 | 1.00 | 22.38 |
| ATOM | 8133 | CE1 | PHE | 1219 | −18.253 | −17.495 | 53.719 | 1.00 | 20.07 |
| ATOM | 8134 | CE2 | PHE | 1219 | −20.612 | −17.918 | 53.983 | 1.00 | 20.42 |
| ATOM | 8135 | CZ | PHE | 1219 | −19.575 | −17.098 | 53.539 | 1.00 | 22.14 |
| ATOM | 8136 | C | PHE | 1219 | −17.384 | −19.811 | 57.411 | 1.00 | 24.16 |
| ATOM | 8137 | O | PHE | 1219 | −17.476 | −18.588 | 57.576 | 1.00 | 20.50 |
| ATOM | 8138 | N | ALA | 1220 | −16.238 | −20.474 | 57.541 | 1.00 | 23.05 |
| ATOM | 8139 | CA | ALA | 1220 | −14.992 | −19.801 | 57.908 | 1.00 | 23.62 |
| ATOM | 8140 | CB | ALA | 1220 | −14.195 | −20.681 | 58.867 | 1.00 | 23.36 |
| ATOM | 8141 | C | ALA | 1220 | −14.134 | −19.450 | 56.698 | 1.00 | 20.64 |
| ATOM | 8142 | O | ALA | 1220 | −14.081 | −20.197 | 55.729 | 1.00 | 20.76 |
| ATOM | 8143 | N | THR | 1221 | −13.462 | −18.309 | 56.773 | 1.00 | 20.56 |
| ATOM | 8144 | CA | THR | 1221 | −12.593 | −17.846 | 55.700 | 1.00 | 21.27 |
| ATOM | 8145 | CB | THR | 1221 | −13.248 | −16.670 | 54.950 | 1.00 | 22.71 |
| ATOM | 8146 | OG1 | THR | 1221 | −14.532 | −17.084 | 54.456 | 1.00 | 25.31 |
| ATOM | 8147 | CG2 | THR | 1221 | −12.391 | −16.229 | 53.790 | 1.00 | 26.49 |
| ATOM | 8148 | C | THR | 1221 | −11.271 | −17.393 | 56.329 | 1.00 | 20.19 |
| ATOM | 8149 | O | THR | 1221 | −11.225 | −17.055 | 57.515 | 1.00 | 19.48 |
| ATOM | 8150 | N | ILE | 1222 | −10.192 | −17.369 | 55.552 | 1.00 | 20.17 |
| ATOM | 8151 | CA | ILE | 1222 | −8.912 | −16.975 | 56.129 | 1.00 | 18.40 |
| ATOM | 8152 | CB | ILE | 1222 | −8.229 | −18.211 | 56.779 | 1.00 | 19.49 |
| ATOM | 8153 | CG2 | ILE | 1222 | −7.614 | −19.105 | 55.696 | 1.00 | 20.26 |
| ATOM | 8154 | CG1 | ILE | 1222 | −7.184 | −17.753 | 57.794 | 1.00 | 21.68 |
| ATOM | 8155 | CD1 | ILE | 1222 | −6.591 | −18.886 | 58.621 | 1.00 | 25.50 |
| ATOM | 8156 | C | ILE | 1222 | −7.972 | −16.338 | 55.115 | 1.00 | 18.20 |
| ATOM | 8157 | O | ILE | 1222 | −8.138 | −16.510 | 53.910 | 1.00 | 17.49 |
| ATOM | 8158 | N | THR | 1223 | −6.998 | −15.575 | 55.602 | 1.00 | 19.22 |
| ATOM | 8159 | CA | THR | 1223 | −6.049 | −14.950 | 54.695 | 1.00 | 19.12 |
| ATOM | 8160 | CB | THR | 1223 | −5.544 | −13.590 | 55.210 | 1.00 | 19.60 |
| ATOM | 8161 | OG1 | THR | 1223 | −4.681 | −13.785 | 56.332 | 1.00 | 20.12 |
| ATOM | 8162 | CG2 | THR | 1223 | −6.719 | −12.709 | 55.625 | 1.00 | 23.60 |
| ATOM | 8163 | C | THR | 1223 | −4.870 | −15.900 | 54.524 | 1.00 | 16.85 |
| ATOM | 8164 | O | THR | 1223 | −4.588 | −16.719 | 55.398 | 1.00 | 16.75 |
| ATOM | 8165 | N | ALA | 1224 | −4.196 | −15.801 | 53.386 | 1.00 | 16.57 |
| ATOM | 8166 | CA | ALA | 1224 | −3.042 | −16.643 | 53.098 | 1.00 | 16.77 |
| ATOM | 8167 | CB | ALA | 1224 | −3.494 | −17.980 | 52.530 | 1.00 | 16.53 |
| ATOM | 8168 | C | ALA | 1224 | −2.181 | −15.886 | 52.099 | 1.00 | 15.47 |
| ATOM | 8169 | O | ALA | 1224 | −2.704 | −15.171 | 51.243 | 1.00 | 13.50 |
| ATOM | 8170 | N | TYR | 1225 | −0.864 | −16.043 | 52.203 | 1.00 | 13.85 |
| ATOM | 8171 | CA | TYR | 1225 | 0.043 | −15.321 | 51.318 | 1.00 | 14.02 |
| ATOM | 8172 | CB | TYR | 1225 | 0.616 | −14.088 | 52.033 | 1.00 | 16.12 |
| ATOM | 8173 | CG | TYR | 1225 | −0.361 | −13.344 | 52.918 | 1.00 | 16.49 |
| ATOM | 8174 | CD1 | TYR | 1225 | −0.398 | −13.577 | 54.293 | 1.00 | 17.47 |
| ATOM | 8175 | CE1 | TYR | 1225 | −1.295 | −12.901 | 55.118 | 1.00 | 19.34 |
| ATOM | 8176 | CD2 | TYR | 1225 | −1.250 | −12.411 | 52.382 | 1.00 | 16.05 |
| ATOM | 8177 | CE2 | TYR | 1225 | −2.155 | −11.728 | 53.197 | 1.00 | 17.27 |
| ATOM | 8178 | CZ | TYR | 1225 | −2.170 | −11.982 | 54.566 | 1.00 | 17.92 |
| ATOM | 8179 | OH | TYR | 1225 | −3.073 | −11.324 | 55.374 | 1.00 | 18.55 |
| ATOM | 8180 | C | TYR | 1225 | 1.204 | −16.158 | 50.807 | 1.00 | 15.21 |
| ATOM | 8181 | O | TYR | 1225 | 2.120 | −15.630 | 50.183 | 1.00 | 16.68 |
| ATOM | 8182 | N | ASP | 1226 | 1.173 | −17.458 | 51.071 | 1.00 | 14.35 |
| ATOM | 8183 | CA | ASP | 1226 | 2.251 | −18.321 | 50.620 | 1.00 | 14.15 |
| ATOM | 8184 | CB | ASP | 1226 | 3.420 | −18.262 | 51.615 | 1.00 | 12.62 |
| ATOM | 8185 | CG | ASP | 1226 | 3.067 | −18.827 | 52.983 | 1.00 | 15.38 |
| ATOM | 8186 | OD1 | ASP | 1226 | 2.953 | −20.064 | 53.099 | 1.00 | 14.85 |
| ATOM | 8187 | OD2 | ASP | 1226 | 2.912 | −18.025 | 53.933 | 1.00 | 15.68 |
| ATOM | 8188 | C | ASP | 1226 | 1.771 | −19.749 | 50.435 | 1.00 | 14.71 |
| ATOM | 8189 | O | ASP | 1226 | 0.672 | −20.113 | 50.864 | 1.00 | 13.48 |
| ATOM | 8190 | N | TYR | 1227 | 2.604 | −20.548 | 49.780 | 1.00 | 13.68 |
| ATOM | 8191 | CA | TYR | 1227 | 2.312 | −21.944 | 49.490 | 1.00 | 12.77 |
| ATOM | 8192 | CB | TYR | 1227 | 3.454 | −22.526 | 48.657 | 1.00 | 13.53 |
| ATOM | 8193 | CG | TYR | 1227 | 3.407 | −24.026 | 48.496 | 1.00 | 14.63 |
| ATOM | 8194 | CD1 | TYR | 1227 | 2.642 | −24.611 | 47.487 | 1.00 | 15.74 |
| ATOM | 8195 | CE1 | TYR | 1227 | 2.594 | −25.990 | 47.324 | 1.00 | 16.62 |
| ATOM | 8196 | CD2 | TYR | 1227 | 4.128 | −24.864 | 49.351 | 1.00 | 17.93 |
| ATOM | 8197 | CE2 | TYR | 1227 | 4.087 | −26.250 | 49.198 | 1.00 | 16.82 |
| ATOM | 8198 | CZ | TYR | 1227 | 3.325 | −26.805 | 48.188 | 1.00 | 19.67 |
| ATOM | 8199 | OH | TYR | 1227 | 3.296 | −28.172 | 48.016 | 1.00 | 20.34 |
| ATOM | 8200 | C | TYR | 1227 | 2.104 | −22.826 | 50.721 | 1.00 | 14.61 |
| ATOM | 8201 | O | TYR | 1227 | 1.169 | −23.631 | 50.770 | 1.00 | 13.84 |
| ATOM | 8202 | N | SER | 1228 | 2.985 | −22.677 | 51.704 | 1.00 | 15.14 |
| ATOM | 8203 | CA | SER | 1228 | 2.932 | −23.488 | 52.915 | 1.00 | 17.24 |
| ATOM | 8204 | CB | SER | 1228 | 4.115 | −23.150 | 53.821 | 1.00 | 17.68 |
| ATOM | 8205 | OG | SER | 1228 | 5.330 | −23.536 | 53.206 | 1.00 | 20.49 |
| ATOM | 8206 | C | SER | 1228 | 1.637 | −23.402 | 53.704 | 1.00 | 16.77 |
| ATOM | 8207 | O | SER | 1228 | 1.012 | −24.425 | 53.989 | 1.00 | 17.76 |
| ATOM | 8208 | N | PHE | 1229 | 1.233 | −22.194 | 54.070 | 1.00 | 17.16 |
| ATOM | 8209 | CA | PHE | 1229 | −0.001 | −22.054 | 54.822 | 1.00 | 16.83 |
| ATOM | 8210 | CB | PHE | 1229 | −0.100 | −20.663 | 55.456 | 1.00 | 17.59 |
| ATOM | 8211 | CG | PHE | 1229 | 0.713 | −20.521 | 56.714 | 1.00 | 17.48 |
| ATOM | 8212 | CD1 | PHE | 1229 | 1.969 | −19.927 | 56.690 | 1.00 | 19.55 |
| ATOM | 8213 | CD2 | PHE | 1229 | 0.239 | −21.041 | 57.916 | 1.00 | 20.98 |
| ATOM | 8214 | CE1 | PHE | 1229 | 2.749 | −19.852 | 57.844 | 1.00 | 19.23 |
| ATOM | 8215 | CE2 | PHE | 1229 | 1.009 | −20.974 | 59.076 | 1.00 | 21.94 |
| ATOM | 8216 | CZ | PHE | 1229 | 2.267 | −20.378 | 59.038 | 1.00 | 20.88 |
| ATOM | 8217 | C | PHE | 1229 | −1.226 | −22.352 | 53.965 | 1.00 | 16.51 |
| ATOM | 8218 | O | PHE | 1229 | −2.190 | −22.929 | 54.452 | 1.00 | 16.61 |
| ATOM | 8219 | N | ALA | 1230 | −1.198 | −21.972 | 52.691 | 1.00 | 15.96 |
| ATOM | 8220 | CA | ALA | 1230 | −2.340 | −22.247 | 51.822 | 1.00 | 17.17 |
| ATOM | 8221 | CB | ALA | 1230 | −2.103 | −21.654 | 50.425 | 1.00 | 15.08 |
| ATOM | 8222 | C | ALA | 1230 | −2.576 | −23.755 | 51.723 | 1.00 | 17.32 |
| ATOM | 8223 | O | ALA | 1230 | −3.715 | −24.216 | 51.757 | 1.00 | 18.17 |
| ATOM | 8224 | N | LYS | 1231 | −1.493 | −24.518 | 51.600 | 1.00 | 17.85 |
| ATOM | 8225 | CA | LYS | 1231 | −1.577 | −25.971 | 51.493 | 1.00 | 18.56 |
| ATOM | 8226 | CB | LYS | 1231 | −0.186 | −26.546 | 51.201 | 1.00 | 19.63 |
| ATOM | 8227 | CG | LYS | 1231 | −0.100 | −28.073 | 51.122 | 1.00 | 23.84 |
| ATOM | 8228 | CD | LYS | 1231 | −0.869 | −28.632 | 49.940 | 1.00 | 29.01 |
| ATOM | 8229 | CE | LYS | 1231 | −0.484 | −30.086 | 49.634 | 1.00 | 31.79 |
| ATOM | 8230 | NZ | LYS | 1231 | −0.862 | −31.058 | 50.706 | 1.00 | 35.95 |
| ATOM | 8231 | C | LYS | 1231 | −2.127 | −26.564 | 52.789 | 1.00 | 17.93 |
| ATOM | 8232 | O | LYS | 1231 | −2.990 | −27.445 | 52.767 | 1.00 | 19.83 |
| ATOM | 8233 | N | LEU | 1232 | −1.626 | −26.072 | 53.916 | 1.00 | 19.47 |
| ATOM | 8234 | CA | LEU | 1232 | −2.063 | −26.557 | 55.219 | 1.00 | 18.49 |
| ATOM | 8235 | CB | LEU | 1232 | −1.265 | −25.861 | 56.324 | 1.00 | 20.15 |
| ATOM | 8236 | CG | LEU | 1232 | −1.474 | −26.366 | 57.758 | 1.00 | 20.23 |
| ATOM | 8237 | CD1 | LEU | 1232 | −0.235 | −26.105 | 58.581 | 1.00 | 20.74 |
| ATOM | 8238 | CD2 | LEU | 1232 | −2.691 | −25.670 | 58.358 | 1.00 | 20.65 |
| ATOM | 8239 | C | LEU | 1232 | −3.566 | −26.318 | 55.394 | 1.00 | 19.99 |
| ATOM | 8240 | O | LEU | 1232 | −4.311 | −27.227 | 55.777 | 1.00 | 19.78 |
| ATOM | 8241 | N | PHE | 1233 | −4.013 | −25.102 | 55.095 | 1.00 | 18.10 |
| ATOM | 8242 | CA | PHE | 1233 | −5.430 | −24.775 | 55.222 | 1.00 | 20.73 |
| ATOM | 8243 | CB | PHE | 1233 | −5.701 | −23.309 | 54.844 | 1.00 | 18.29 |
| ATOM | 8244 | CG | PHE | 1233 | −4.957 | −22.306 | 55.680 | 1.00 | 18.77 |
| ATOM | 8245 | CD1 | PHE | 1233 | −4.606 | −22.591 | 56.996 | 1.00 | 18.49 |
| ATOM | 8246 | CD2 | PHE | 1233 | −4.628 | −21.061 | 55.156 | 1.00 | 17.61 |
| ATOM | 8247 | CE1 | PHE | 1233 | −3.940 | −21.652 | 57.769 | 1.00 | 17.42 |
| ATOM | 8248 | CE2 | PHE | 1233 | −3.961 | −20.117 | 55.926 | 1.00 | 15.70 |
| ATOM | 8249 | CZ | PHE | 1233 | −3.618 | −20.413 | 57.233 | 1.00 | 17.50 |
| ATOM | 8250 | C | PHE | 1233 | −6.278 | −25.676 | 54.336 | 1.00 | 21.36 |
| ATOM | 8251 | O | PHE | 1233 | −7.299 | −26.215 | 54.775 | 1.00 | 22.69 |
| ATOM | 8252 | N | ALA | 1234 | −5.859 | −25.840 | 53.084 | 1.00 | 21.60 |
| ATOM | 8253 | CA | ALA | 1234 | −6.601 | −26.679 | 52.147 | 1.00 | 21.61 |
| ATOM | 8254 | CB | ALA | 1234 | −5.960 | −26.612 | 50.758 | 1.00 | 21.86 |
| ATOM | 8255 | C | ALA | 1234 | −6.709 | −28.133 | 52.612 | 1.00 | 23.65 |
| ATOM | 8256 | O | ALA | 1234 | −7.759 | −28.758 | 52.460 | 1.00 | 19.72 |
| ATOM | 8257 | N | ASP | 1235 | −5.634 | −28.675 | 53.179 | 1.00 | 23.15 |
| ATOM | 8258 | CA | ASP | 1235 | −5.674 | −30.061 | 53.646 | 1.00 | 26.64 |
| ATOM | 8259 | CB | ASP | 1235 | −4.264 | −30.577 | 53.973 | 1.00 | 26.19 |
| ATOM | 8260 | CG | ASP | 1235 | −3.330 | −30.562 | 52.767 | 1.00 | 30.30 |
| ATOM | 8261 | OD1 | ASP | 1235 | −3.758 | −30.941 | 51.657 | 1.00 | 29.31 |
| ATOM | 8262 | OD2 | ASP | 1235 | −2.153 | −30.185 | 52.941 | 1.00 | 30.67 |
| ATOM | 8263 | C | ASP | 1235 | −6.561 | −30.216 | 54.884 | 1.00 | 25.67 |
| ATOM | 8264 | O | ASP | 1235 | −6.896 | −31.333 | 55.275 | 1.00 | 27.41 |
| ATOM | 8265 | N | GLU | 1236 | −6.935 | −29.098 | 55.500 | 1.00 | 26.80 |
| ATOM | 8266 | CA | GLU | 1236 | −7.779 | −29.131 | 56.693 | 1.00 | 28.17 |
| ATOM | 8267 | CB | GLU | 1236 | −7.236 | −28.167 | 57.749 | 1.00 | 28.31 |
| ATOM | 8268 | CG | GLU | 1236 | −5.900 | −28.583 | 58.322 | 1.00 | 30.31 |
| ATOM | 8269 | CD | GLU | 1236 | −5.947 | −29.977 | 58.913 | 1.00 | 30.97 |
| ATOM | 8270 | OE1 | GLU | 1236 | −6.872 | −30.252 | 59.709 | 1.00 | 32.20 |
| ATOM | 8271 | OE2 | GLU | 1236 | −5.066 | −30.797 | 58.587 | 1.00 | 34.13 |
| ATOM | 8272 | C | GLU | 1236 | −9.242 | −28.805 | 56.407 | 1.00 | 29.14 |
| ATOM | 8273 | O | GLU | 1236 | −10.098 | −28.956 | 57.277 | 1.00 | 29.70 |
| ATOM | 8274 | N | GLY | 1237 | −9.531 | −28.348 | 55.194 | 1.00 | 28.96 |
| ATOM | 8275 | CA | GLY | 1237 | −10.908 | −28.032 | 54.863 | 1.00 | 30.43 |
| ATOM | 8276 | C | GLY | 1237 | −11.165 | −26.570 | 54.567 | 1.00 | 29.93 |
| ATOM | 8277 | O | GLY | 1237 | −12.165 | −26.248 | 53.937 | 1.00 | 32.59 |
| ATOM | 8278 | N | LEU | 1238 | −10.286 | −25.684 | 55.033 | 1.00 | 28.83 |
| ATOM | 8279 | CA | LEU | 1238 | −10.449 | −24.257 | 54.771 | 1.00 | 28.65 |
| ATOM | 8280 | CB | LEU | 1238 | −9.434 | −23.438 | 55.568 | 1.00 | 28.60 |
| ATOM | 8281 | CG | LEU | 1238 | −9.946 | −22.829 | 56.866 | 1.00 | 30.97 |
| ATOM | 8282 | CD1 | LEU | 1238 | −8.833 | −22.052 | 57.548 | 1.00 | 29.17 |
| ATOM | 8283 | CD2 | LEU | 1238 | −11.130 | −21.918 | 56.559 | 1.00 | 30.61 |
| ATOM | 8284 | C | LEU | 1238 | −10.225 | −24.039 | 53.283 | 1.00 | 26.94 |
| ATOM | 8285 | O | LEU | 1238 | −9.085 | −23.949 | 52.835 | 1.00 | 27.39 |
| ATOM | 8286 | N | ASN | 1239 | −11.313 | −23.948 | 52.523 | 1.00 | 24.94 |
| ATOM | 8287 | CA | ASN | 1239 | −11.206 | −23.782 | 51.080 | 1.00 | 24.45 |
| ATOM | 8288 | CB | ASN | 1239 | −12.093 | −24.803 | 50.369 | 1.00 | 26.67 |
| ATOM | 8289 | CG | ASN | 1239 | −11.838 | −26.213 | 50.844 | 1.00 | 29.98 |
| ATOM | 8290 | OD1 | ASN | 1239 | −10.690 | −26.664 | 50.903 | 1.00 | 31.35 |
| ATOM | 8291 | ND2 | ASN | 1239 | −12.905 | −26.924 | 51.188 | 1.00 | 33.26 |
| ATOM | 8292 | C | ASN | 1239 | −11.527 | −22.391 | 50.573 | 1.00 | 22.89 |
| ATOM | 8293 | O | ASN | 1239 | −11.763 | −22.201 | 49.382 | 1.00 | 24.50 |
| ATOM | 8294 | N | VAL | 1240 | −11.545 | −21.420 | 51.475 | 1.00 | 21.81 |
| ATOM | 8295 | CA | VAL | 1240 | −11.806 | −20.039 | 51.086 | 1.00 | 19.80 |
| ATOM | 8296 | CB | VAL | 1240 | −13.137 | −19.519 | 51.629 | 1.00 | 20.14 |
| ATOM | 8297 | CG1 | VAL | 1240 | −13.358 | −18.098 | 51.141 | 1.00 | 18.87 |
| ATOM | 8298 | CG2 | VAL | 1240 | −14.263 | −20.413 | 51.176 | 1.00 | 17.75 |
| ATOM | 8299 | C | VAL | 1240 | −10.691 | −19.190 | 51.663 | 1.00 | 19.46 |
| ATOM | 8300 | O | VAL | 1240 | −10.632 | −18.960 | 52.868 | 1.00 | 18.98 |
| ATOM | 8301 | N | MET | 1241 | −9.811 | −18.719 | 50.788 | 1.00 | 18.30 |
| ATOM | 8302 | CA | MET | 1241 | −8.678 | −17.922 | 51.210 | 1.00 | 17.27 |
| ATOM | 8303 | CB | MET | 1241 | −7.382 | −18.672 | 50.927 | 1.00 | 17.64 |
| ATOM | 8304 | CG | MET | 1241 | −7.204 | −19.922 | 51.769 | 1.00 | 18.74 |
| ATOM | 8305 | SD | MET | 1241 | −5.840 | −20.926 | 51.216 | 1.00 | 19.35 |
| ATOM | 8306 | CE | MET | 1241 | −6.619 | −22.510 | 51.062 | 1.00 | 19.09 |
| ATOM | 8307 | C | MET | 1241 | −8.629 | −16.583 | 50.521 | 1.00 | 16.92 |
| ATOM | 8308 | O | MET | 1241 | −8.975 | −16.469 | 49.348 | 1.00 | 17.62 |
| ATOM | 8309 | N | LEU | 1242 | −8.171 | −15.571 | 51.249 | 1.00 | 16.36 |
| ATOM | 8310 | CA | LEU | 1242 | −8.069 | −14.233 | 50.694 | 1.00 | 17.87 |
| ATOM | 8311 | CB | LEU | 1242 | −8.950 | −13.270 | 51.501 | 1.00 | 19.81 |
| ATOM | 8312 | CG | LEU | 1242 | −9.047 | −11.757 | 51.253 | 1.00 | 23.91 |
| ATOM | 8313 | CD1 | LEU | 1242 | −7.919 | −11.072 | 51.987 | 1.00 | 25.91 |
| ATOM | 8314 | CD2 | LEU | 1242 | −9.038 | −11.402 | 49.770 | 1.00 | 21.85 |
| ATOM | 8315 | C | LEU | 1242 | −6.625 | −13.759 | 50.682 | 1.00 | 16.01 |
| ATOM | 8316 | O | LEU | 1242 | −5.911 | −13.860 | 51.679 | 1.00 | 14.63 |
| ATOM | 8317 | N | VAL | 1243 | −6.202 | −13.259 | 49.529 | 1.00 | 16.58 |
| ATOM | 8318 | CA | VAL | 1243 | −4.859 | −12.724 | 49.381 | 1.00 | 15.62 |
| ATOM | 8319 | CB | VAL | 1243 | −4.233 | −13.152 | 48.046 | 1.00 | 15.84 |
| ATOM | 8320 | CG1 | VAL | 1243 | −2.801 | −12.644 | 47.954 | 1.00 | 15.84 |
| ATOM | 8321 | CG2 | VAL | 1243 | −4.274 | −14.668 | 47.923 | 1.00 | 17.35 |
| ATOM | 8322 | C | VAL | 1243 | −5.086 | −11.223 | 49.413 | 1.00 | 15.28 |
| ATOM | 8323 | O | VAL | 1243 | −5.272 | −10.590 | 48.376 | 1.00 | 14.94 |
| ATOM | 8324 | N | GLY | 1244 | −5.085 | −10.662 | 50.622 | 1.00 | 16.14 |
| ATOM | 8325 | CA | GLY | 1244 | −5.351 | −9.244 | 50.780 | 1.00 | 18.27 |
| ATOM | 8326 | C | GLY | 1244 | −4.156 | −8.347 | 50.980 | 1.00 | 16.28 |
| ATOM | 8327 | O | GLY | 1244 | −3.061 | −8.815 | 51.306 | 1.00 | 14.05 |
| ATOM | 8328 | N | ASP | 1245 | −4.368 | −7.046 | 50.798 | 1.00 | 17.20 |
| ATOM | 8329 | CA | ASP | 1245 | −3.276 | −6.105 | 50.957 | 1.00 | 17.71 |
| ATOM | 8330 | CB | ASP | 1245 | −3.620 | −4.728 | 50.369 | 1.00 | 17.04 |
| ATOM | 8331 | CG | ASP | 1245 | −4.889 | −4.121 | 50.952 | 1.00 | 16.86 |
| ATOM | 8332 | OD1 | ASP | 1245 | −5.415 | −4.640 | 51.951 | 1.00 | 16.51 |
| ATOM | 8333 | OD2 | ASP | 1245 | −5.338 | −3.111 | 50.384 | 1.00 | 17.00 |
| ATOM | 8334 | C | ASP | 1245 | −2.837 | −5.978 | 52.404 | 1.00 | 16.69 |
| ATOM | 8335 | O | ASP | 1245 | −1.944 | −5.202 | 52.715 | 1.00 | 15.57 |
| ATOM | 8336 | N | SER | 1246 | −3.471 | −6.736 | 53.291 | 1.00 | 16.51 |
| ATOM | 8337 | CA | SER | 1246 | −3.050 | −6.722 | 54.685 | 1.00 | 18.08 |
| ATOM | 8338 | CB | SER | 1246 | −3.963 | −7.606 | 55.539 | 1.00 | 17.32 |
| ATOM | 8339 | OG | SER | 1246 | −4.087 | −8.920 | 55.004 | 1.00 | 20.35 |
| ATOM | 8340 | C | SER | 1246 | −1.617 | −7.262 | 54.687 | 1.00 | 17.95 |
| ATOM | 8341 | O | SER | 1246 | −0.865 | −7.062 | 55.642 | 1.00 | 18.43 |
| ATOM | 8342 | N | LEU | 1247 | −1.234 | −7.943 | 53.606 | 1.00 | 17.78 |
| ATOM | 8343 | CA | LEU | 1247 | 0.131 | −8.473 | 53.503 | 1.00 | 17.35 |
| ATOM | 8344 | CB | LEU | 1247 | 0.302 | −9.318 | 52.225 | 1.00 | 16.63 |
| ATOM | 8345 | CG | LEU | 1247 | 0.200 | −8.648 | 50.854 | 1.00 | 14.50 |
| ATOM | 8346 | CD1 | LEU | 1247 | 1.513 | −7.956 | 50.524 | 1.00 | 14.50 |
| ATOM | 8347 | CD2 | LEU | 1247 | −0.135 | −9.688 | 49.777 | 1.00 | 14.80 |
| ATOM | 8348 | C | LEU | 1247 | 1.143 | −7.323 | 53.521 | 1.00 | 15.79 |
| ATOM | 8349 | O | LEU | 1247 | 2.330 | −7.520 | 53.796 | 1.00 | 14.98 |
| ATOM | 8350 | N | GLY | 1248 | 0.682 | −6.116 | 53.212 | 1.00 | 13.56 |
| ATOM | 8351 | CA | GLY | 1248 | 1.584 | −4.978 | 53.226 | 1.00 | 13.97 |
| ATOM | 8352 | C | GLY | 1248 | 2.119 | −4.763 | 54.624 | 1.00 | 15.47 |
| ATOM | 8353 | O | GLY | 1248 | 3.228 | −4.261 | 54.820 | 1.00 | 16.88 |
| ATOM | 8354 | N | MET | 1249 | 1.325 | −5.162 | 55.608 | 1.00 | 14.33 |
| ATOM | 8355 | CA | MET | 1249 | 1.719 | −5.005 | 56.996 | 1.00 | 16.01 |
| ATOM | 8356 | CB | MET | 1249 | 0.513 | −4.502 | 57.800 | 1.00 | 19.46 |
| ATOM | 8357 | CG | MET | 1249 | 0.018 | −3.144 | 57.322 | 1.00 | 21.69 |
| ATOM | 8358 | SD | MET | 1249 | −1.516 | −2.566 | 58.097 | 1.00 | 26.00 |
| ATOM | 8359 | CE | MET | 1249 | −0.965 | −2.281 | 59.756 | 1.00 | 27.35 |
| ATOM | 8360 | C | MET | 1249 | 2.290 | −6.289 | 57.602 | 1.00 | 16.59 |
| ATOM | 8361 | O | MET | 1249 | 3.417 | −6.305 | 58.096 | 1.00 | 14.81 |
| ATOM | 8362 | N | THR | 1250 | 1.525 | −7.369 | 57.530 | 1.00 | 16.12 |
| ATOM | 8363 | CA | THR | 1250 | 1.946 | −8.636 | 58.100 | 1.00 | 18.02 |
| ATOM | 8364 | CB | THR | 1250 | 0.751 | −9.598 | 58.167 | 1.00 | 19.93 |
| ATOM | 8365 | OG1 | THR | 1250 | 1.098 | −10.759 | 58.930 | 1.00 | 25.56 |
| ATOM | 8366 | CG2 | THR | 1250 | 0.341 | −10.019 | 56.777 | 1.00 | 21.14 |
| ATOM | 8367 | C | THR | 1250 | 3.111 | −9.311 | 57.368 | 1.00 | 16.42 |
| ATOM | 8368 | O | THR | 1250 | 3.920 | −10.011 | 57.982 | 1.00 | 17.25 |
| ATOM | 8369 | N | VAL | 1251 | 3.207 | −9.114 | 56.060 | 1.00 | 14.38 |
| ATOM | 8370 | CA | VAL | 1251 | 4.295 | −9.729 | 55.306 | 1.00 | 14.53 |
| ATOM | 8371 | CB | VAL | 1251 | 3.792 | −10.309 | 53.953 | 1.00 | 15.45 |
| ATOM | 8372 | CG1 | VAL | 1251 | 4.975 | −10.833 | 53.133 | 1.00 | 12.75 |
| ATOM | 8373 | CG2 | VAL | 1251 | 2.796 | −11.428 | 54.210 | 1.00 | 15.08 |
| ATOM | 8374 | C | VAL | 1251 | 5.419 | −8.732 | 55.039 | 1.00 | 14.56 |
| ATOM | 8375 | O | VAL | 1251 | 6.573 | −8.978 | 55.395 | 1.00 | 16.60 |
| ATOM | 8376 | N | GLN | 1252 | 5.077 | −7.598 | 54.437 | 1.00 | 11.43 |
| ATOM | 8377 | CA | GLN | 1252 | 6.064 | −6.574 | 54.097 | 1.00 | 13.81 |
| ATOM | 8378 | CB | GLN | 1252 | 5.493 | −5.649 | 53.026 | 1.00 | 14.06 |
| ATOM | 8379 | CG | GLN | 1252 | 4.925 | −6.404 | 51.832 | 1.00 | 15.86 |
| ATOM | 8380 | CD | GLN | 1252 | 4.458 | −5.482 | 50.727 | 1.00 | 14.76 |
| ATOM | 8381 | OE1 | GLN | 1252 | 4.178 | −4.304 | 50.960 | 1.00 | 14.09 |
| ATOM | 8382 | NE2 | GLN | 1252 | 4.358 | −6.019 | 49.513 | 1.00 | 12.71 |
| ATOM | 8383 | C | GLN | 1252 | 6.581 | −5.742 | 55.270 | 1.00 | 14.83 |
| ATOM | 8384 | O | GLN | 1252 | 7.726 | −5.291 | 55.249 | 1.00 | 15.58 |
| ATOM | 8385 | N | GLY | 1253 | 5.739 | −5.523 | 56.272 | 1.00 | 15.09 |
| ATOM | 8386 | CA | GLY | 1253 | 6.165 | −4.760 | 57.434 | 1.00 | 15.66 |
| ATOM | 8387 | C | GLY | 1253 | 5.888 | −3.269 | 57.397 | 1.00 | 17.26 |
| ATOM | 8388 | O | GLY | 1253 | 6.480 | −2.501 | 58.163 | 1.00 | 18.18 |
| ATOM | 8389 | N | HIS | 1254 | 5.001 | −2.838 | 56.509 | 1.00 | 17.51 |
| ATOM | 8390 | CA | HIS | 1254 | 4.671 | −1.420 | 56.429 | 1.00 | 18.70 |
| ATOM | 8391 | CB | HIS | 1254 | 4.126 | −1.065 | 55.045 | 1.00 | 19.45 |
| ATOM | 8392 | CG | HIS | 1254 | 5.119 | −1.251 | 53.945 | 1.00 | 19.97 |
| ATOM | 8393 | CD2 | HIS | 1254 | 5.122 | −2.083 | 52.876 | 1.00 | 19.30 |
| ATOM | 8394 | ND1 | HIS | 1254 | 6.287 | −0.522 | 53.868 | 1.00 | 18.30 |
| ATOM | 8395 | CE1 | HIS | 1254 | 6.965 | −0.896 | 52.798 | 1.00 | 22.21 |
| ATOM | 8396 | NE2 | HIS | 1254 | 6.280 | −1.841 | 52.178 | 1.00 | 21.62 |
| ATOM | 8397 | C | HIS | 1254 | 3.634 | −1.094 | 57.488 | 1.00 | 18.59 |
| ATOM | 8398 | O | HIS | 1254 | 2.986 | −1.996 | 58.025 | 1.00 | 19.51 |
| ATOM | 8399 | N | ASP | 1255 | 3.487 | 0.192 | 57.788 | 1.00 | 19.71 |
| ATOM | 8400 | CA | ASP | 1255 | 2.530 | 0.651 | 58.795 | 1.00 | 23.34 |
| ATOM | 8401 | CB | ASP | 1255 | 2.943 | 2.030 | 59.331 | 1.00 | 27.63 |
| ATOM | 8402 | CG | ASP | 1255 | 3.048 | 3.077 | 58.239 | 1.00 | 29.81 |
| ATOM | 8403 | OD1 | ASP | 1255 | 2.101 | 3.214 | 57.443 | 1.00 | 33.96 |
| ATOM | 8404 | OD2 | ASP | 1255 | 4.082 | 3.776 | 58.178 | 1.00 | 37.20 |
| ATOM | 8405 | C | ASP | 1255 | 1.096 | 0.712 | 58.272 | 1.00 | 21.29 |
| ATOM | 8406 | O | ASP | 1255 | 0.156 | 0.883 | 59.047 | 1.00 | 21.48 |
| ATOM | 8407 | N | SER | 1256 | 0.931 | 0.587 | 56.958 | 1.00 | 19.07 |
| ATOM | 8408 | CA | SER | 1256 | −0.398 | 0.609 | 56.339 | 1.00 | 16.19 |
| ATOM | 8409 | CB | SER | 1256 | −0.761 | 2.024 | 55.869 | 1.00 | 14.13 |
| ATOM | 8410 | OG | SER | 1256 | 0.016 | 2.418 | 54.748 | 1.00 | 13.20 |
| ATOM | 8411 | C | SER | 1256 | −0.394 | −0.339 | 55.141 | 1.00 | 16.00 |
| ATOM | 8412 | O | SER | 1256 | 0.628 | −0.956 | 54.838 | 1.00 | 16.64 |
| ATOM | 8413 | N | THR | 1257 | −1.526 | −0.444 | 54.453 | 1.00 | 15.54 |
| ATOM | 8414 | CA | THR | 1257 | −1.616 | −1.327 | 53.296 | 1.00 | 13.85 |
| ATOM | 8415 | CB | THR | 1257 | −3.001 | −2.009 | 53.214 | 1.00 | 14.68 |
| ATOM | 8416 | OG1 | THR | 1257 | −4.005 | −1.020 | 52.942 | 1.00 | 13.16 |
| ATOM | 8417 | CG2 | THR | 1257 | −3.333 | −2.708 | 54.518 | 1.00 | 14.28 |
| ATOM | 8418 | C | THR | 1257 | −1.383 | −0.596 | 51.978 | 1.00 | 14.73 |
| ATOM | 8419 | O | THR | 1257 | −1.260 | −1.232 | 50.931 | 1.00 | 14.37 |
| ATOM | 8420 | N | LEU | 1258 | −1.296 | 0.731 | 52.028 | 1.00 | 11.56 |
| ATOM | 8421 | CA | LEU | 1258 | −1.116 | 1.522 | 50.810 | 1.00 | 14.62 |
| ATOM | 8422 | CB | LEU | 1258 | −1.059 | 3.022 | 51.151 | 1.00 | 14.76 |
| ATOM | 8423 | CG | LEU | 1258 | −2.415 | 3.708 | 51.387 | 1.00 | 18.81 |
| ATOM | 8424 | CD1 | LEU | 1258 | −3.037 | 3.193 | 52.689 | 1.00 | 18.31 |
| ATOM | 8425 | CD2 | LEU | 1258 | −2.225 | 5.211 | 51.451 | 1.00 | 17.89 |
| ATOM | 8426 | C | LEU | 1258 | 0.049 | 1.172 | 49.873 | 1.00 | 13.86 |
| ATOM | 8427 | O | LEU | 1258 | −0.099 | 1.248 | 48.655 | 1.00 | 13.08 |
| ATOM | 8428 | N | PRO | 1259 | 1.215 | 0.790 | 50.419 | 1.00 | 15.27 |
| ATOM | 8429 | CD | PRO | 1259 | 1.610 | 0.817 | 51.836 | 1.00 | 16.92 |
| ATOM | 8430 | CA | PRO | 1259 | 2.355 | 0.448 | 49.562 | 1.00 | 13.94 |
| ATOM | 8431 | CB | PRO | 1259 | 3.521 | 0.372 | 50.544 | 1.00 | 17.82 |
| ATOM | 8432 | CG | PRO | 1259 | 2.850 | −0.036 | 51.830 | 1.00 | 22.73 |
| ATOM | 8433 | C | PRO | 1259 | 2.208 | −0.827 | 48.733 | 1.00 | 13.69 |
| ATOM | 8434 | O | PRO | 1259 | 2.956 | −1.020 | 47.779 | 1.00 | 11.25 |
| ATOM | 8435 | N | VAL | 1260 | 1.254 | −1.689 | 49.081 | 1.00 | 12.02 |
| ATOM | 8436 | CA | VAL | 1260 | 1.061 | −2.932 | 48.330 | 1.00 | 12.65 |
| ATOM | 8437 | CB | VAL | 1260 | −0.025 | −3.825 | 48.981 | 1.00 | 12.45 |
| ATOM | 8438 | CG1 | VAL | 1260 | −0.258 | −5.057 | 48.132 | 1.00 | 11.57 |
| ATOM | 8439 | CG2 | VAL | 1260 | 0.401 | −4.235 | 50.391 | 1.00 | 9.00 |
| ATOM | 8440 | C | VAL | 1260 | 0.659 | −2.631 | 46.892 | 1.00 | 12.91 |
| ATOM | 8441 | O | VAL | 1260 | −0.252 | −1.834 | 46.638 | 1.00 | 12.38 |
| ATOM | 8442 | N | THR | 1261 | 1.325 | −3.281 | 45.941 | 1.00 | 14.02 |
| ATOM | 8443 | CA | THR | 1261 | 1.019 | −3.060 | 44.529 | 1.00 | 13.63 |
| ATOM | 8444 | CB | THR | 1261 | 2.326 | −2.816 | 43.724 | 1.00 | 18.67 |
| ATOM | 8445 | OG1 | THR | 1261 | 3.072 | −1.749 | 44.337 | 1.00 | 18.95 |
| ATOM | 8446 | CG2 | THR | 1261 | 2.010 | −2.410 | 42.285 | 1.00 | 22.02 |
| ATOM | 8447 | C | THR | 1261 | 0.268 | −4.259 | 43.930 | 1.00 | 12.64 |
| ATOM | 8448 | O | THR | 1261 | 0.115 | −5.289 | 44.572 | 1.00 | 11.51 |
| ATOM | 8449 | N | VAL | 1262 | −0.197 | −4.108 | 42.697 | 1.00 | 10.72 |
| ATOM | 8450 | CA | VAL | 1262 | −0.906 | −5.181 | 42.017 | 1.00 | 11.46 |
| ATOM | 8451 | CB | VAL | 1262 | −1.484 | −4.682 | 40.675 | 1.00 | 10.61 |
| ATOM | 8452 | CG1 | VAL | 1262 | −2.069 | −5.852 | 39.875 | 1.00 | 10.47 |
| ATOM | 8453 | CG2 | VAL | 1262 | −2.576 | −3.631 | 40.956 | 1.00 | 10.70 |
| ATOM | 8454 | C | VAL | 1262 | 0.068 | −6.339 | 41.790 | 1.00 | 11.29 |
| ATOM | 8455 | O | VAL | 1262 | −0.310 | −7.511 | 41.895 | 1.00 | 11.57 |
| ATOM | 8456 | N | ALA | 1263 | 1.324 | −6.006 | 41.497 | 1.00 | 12.19 |
| ATOM | 8457 | CA | ALA | 1263 | 2.339 | −7.031 | 41.275 | 1.00 | 11.44 |
| ATOM | 8458 | CB | ALA | 1263 | 3.673 | −6.389 | 40.842 | 1.00 | 11.79 |
| ATOM | 8459 | C | ALA | 1263 | 2.531 | −7.843 | 42.556 | 1.00 | 11.93 |
| ATOM | 8460 | O | ALA | 1263 | 2.724 | −9.065 | 42.505 | 1.00 | 10.47 |
| ATOM | 8461 | N | ASP | 1264 | 2.491 | −7.172 | 43.708 | 1.00 | 9.71 |
| ATOM | 8462 | CA | ASP | 1264 | 2.648 | −7.883 | 44.981 | 1.00 | 13.19 |
| ATOM | 8463 | CB | ASP | 1264 | 2.646 | −6.916 | 46.177 | 1.00 | 12.97 |
| ATOM | 8464 | CG | ASP | 1264 | 3.782 | −5.904 | 46.135 | 1.00 | 15.35 |
| ATOM | 8465 | OD1 | ASP | 1264 | 4.833 | −6.206 | 45.548 | 1.00 | 13.99 |
| ATOM | 8466 | OD2 | ASP | 1264 | 3.635 | −4.805 | 46.713 | 1.00 | 12.70 |
| ATOM | 8467 | C | ASP | 1264 | 1.498 | −8.873 | 45.157 | 1.00 | 11.29 |
| ATOM | 8468 | O | ASP | 1264 | 1.709 | −10.036 | 45.488 | 1.00 | 12.23 |
| ATOM | 8469 | N | ILE | 1265 | 0.279 | −8.406 | 44.920 | 1.00 | 10.36 |
| ATOM | 8470 | CA | ILE | 1265 | −0.886 | −9.263 | 45.053 | 1.00 | 9.25 |
| ATOM | 8471 | CB | ILE | 1265 | −2.194 | −8.492 | 44.733 | 1.00 | 8.35 |
| ATOM | 8472 | CG2 | ILE | 1265 | −3.381 | −9.460 | 44.721 | 1.00 | 10.20 |
| ATOM | 8473 | CG1 | ILE | 1265 | −2.436 | −7.394 | 45.763 | 1.00 | 10.86 |
| ATOM | 8474 | CD1 | ILE | 1265 | −2.621 | −7.897 | 47.180 | 1.00 | 13.04 |
| ATOM | 8475 | C | ILE | 1265 | −0.782 | −10.474 | 44.125 | 1.00 | 10.11 |
| ATOM | 8476 | O | ILE | 1265 | −1.066 | −11.591 | 44.539 | 1.00 | 10.76 |
| ATOM | 8477 | N | ALA | 1266 | −0.373 | −10.259 | 42.878 | 1.00 | 11.37 |
| ATOM | 8478 | CA | ALA | 1266 | −0.269 | −11.361 | 41.915 | 1.00 | 10.83 |
| ATOM | 8479 | CB | ALA | 1266 | −0.007 | −10.804 | 40.512 | 1.00 | 10.92 |
| ATOM | 8480 | C | ALA | 1266 | 0.801 | −12.398 | 42.299 | 1.00 | 10.56 |
| ATOM | 8481 | O | ALA | 1266 | 0.655 | −13.606 | 42.048 | 1.00 | 10.01 |
| ATOM | 8482 | N | TYR | 1267 | 1.882 | −11.919 | 42.905 | 1.00 | 9.93 |
| ATOM | 8483 | CA | TYR | 1267 | 2.978 | −12.783 | 43.360 | 1.00 | 8.50 |
| ATOM | 8484 | CB | TYR | 1267 | 4.122 | −11.912 | 43.911 | 1.00 | 9.28 |
| ATOM | 8485 | CG | TYR | 1267 | 5.236 | −12.676 | 44.598 | 1.00 | 9.52 |
| ATOM | 8486 | CD1 | TYR | 1267 | 6.098 | −13.493 | 43.879 | 1.00 | 11.95 |
| ATOM | 8487 | CE1 | TYR | 1267 | 7.142 | −14.176 | 44.509 | 1.00 | 12.07 |
| ATOM | 8488 | CD2 | TYR | 1267 | 5.436 | −12.559 | 45.972 | 1.00 | 12.37 |
| ATOM | 8489 | CE2 | TYR | 1267 | 6.475 | −13.237 | 46.615 | 1.00 | 12.84 |
| ATOM | 8490 | CZ | TYR | 1267 | 7.321 | −14.039 | 45.875 | 1.00 | 14.28 |
| ATOM | 8491 | OH | TYR | 1267 | 8.365 | −14.691 | 46.510 | 1.00 | 13.09 |
| ATOM | 8492 | C | TYR | 1267 | 2.461 | −13.732 | 44.441 | 1.00 | 9.24 |
| ATOM | 8493 | O | TYR | 1267 | 2.636 | −14.960 | 44.368 | 1.00 | 7.23 |
| ATOM | 8494 | N | HIS | 1268 | 1.808 | −13.162 | 45.448 | 1.00 | 10.07 |
| ATOM | 8495 | CA | HIS | 1268 | 1.277 | −13.968 | 46.549 | 1.00 | 11.40 |
| ATOM | 8496 | CB | HIS | 1268 | 0.899 | −13.042 | 47.717 | 1.00 | 10.91 |
| ATOM | 8497 | CG | HIS | 1268 | 2.093 | −12.471 | 48.425 | 1.00 | 10.67 |
| ATOM | 8498 | CD2 | HIS | 1268 | 2.720 | −11.277 | 48.299 | 1.00 | 12.54 |
| ATOM | 8499 | ND1 | HIS | 1268 | 2.850 | −13.207 | 49.310 | 1.00 | 9.83 |
| ATOM | 8500 | CE1 | HIS | 1268 | 3.896 | −12.495 | 49.694 | 1.00 | 12.36 |
| ATOM | 8501 | NE2 | HIS | 1268 | 3.842 | −11.321 | 49.095 | 1.00 | 11.55 |
| ATOM | 8502 | C | HIS | 1268 | 0.099 | −14.836 | 46.096 | 1.00 | 12.08 |
| ATOM | 8503 | O | HIS | 1268 | −0.104 | −15.933 | 46.610 | 1.00 | 10.75 |
| ATOM | 8504 | N | THR | 1269 | −0.655 | −14.351 | 45.112 | 1.00 | 11.50 |
| ATOM | 8505 | CA | THR | 1269 | −1.794 | −15.094 | 44.591 | 1.00 | 11.48 |
| ATOM | 8506 | CB | THR | 1269 | −2.590 | −14.224 | 43.602 | 1.00 | 11.59 |
| ATOM | 8507 | OG1 | THR | 1269 | −3.215 | −13.149 | 44.319 | 1.00 | 12.48 |
| ATOM | 8508 | CG2 | THR | 1269 | −3.651 | −15.039 | 42.884 | 1.00 | 13.07 |
| ATOM | 8509 | C | THR | 1269 | −1.340 | −16.386 | 43.909 | 1.00 | 10.67 |
| ATOM | 8510 | O | THR | 1269 | −1.928 | −17.443 | 44.119 | 1.00 | 13.58 |
| ATOM | 8511 | N | ALA | 1270 | −0.292 | −16.301 | 43.098 | 1.00 | 8.94 |
| ATOM | 8512 | CA | ALA | 1270 | 0.228 | −17.478 | 42.414 | 1.00 | 10.29 |
| ATOM | 8513 | CB | ALA | 1270 | 1.366 | −17.076 | 41.465 | 1.00 | 11.72 |
| ATOM | 8514 | C | ALA | 1270 | 0.737 | −18.492 | 43.437 | 1.00 | 10.90 |
| ATOM | 8515 | O | ALA | 1270 | 0.556 | −19.701 | 43.269 | 1.00 | 12.02 |
| ATOM | 8516 | N | ALA | 1271 | 1.379 | −17.998 | 44.495 | 1.00 | 11.07 |
| ATOM | 8517 | CA | ALA | 1271 | 1.924 | −18.878 | 45.534 | 1.00 | 11.19 |
| ATOM | 8518 | CB | ALA | 1271 | 2.733 | −18.064 | 46.541 | 1.00 | 10.42 |
| ATOM | 8519 | C | ALA | 1271 | 0.816 | −19.642 | 46.251 | 1.00 | 11.93 |
| ATOM | 8520 | O | ALA | 1271 | 0.912 | −20.849 | 46.458 | 1.00 | 9.16 |
| ATOM | 8521 | N | VAL | 1272 | −0.228 | −18.922 | 46.645 | 1.00 | 12.77 |
| ATOM | 8522 | CA | VAL | 1272 | −1.356 | −19.528 | 47.331 | 1.00 | 12.55 |
| ATOM | 8523 | CB | VAL | 1272 | −2.339 | −18.436 | 47.812 | 1.00 | 10.99 |
| ATOM | 8524 | CG1 | VAL | 1272 | −3.613 | −19.062 | 48.365 | 1.00 | 13.11 |
| ATOM | 8525 | CG2 | VAL | 1272 | −1.660 | −17.597 | 48.893 | 1.00 | 10.80 |
| ATOM | 8526 | C | VAL | 1272 | −2.063 | −20.535 | 46.436 | 1.00 | 12.93 |
| ATOM | 8527 | O | VAL | 1272 | −2.442 | −21.624 | 46.882 | 1.00 | 13.02 |
| ATOM | 8528 | N | ARG | 1273 | −2.226 | −20.191 | 45.163 | 1.00 | 12.86 |
| ATOM | 8529 | CA | ARG | 1273 | −2.883 | −21.109 | 44.236 | 1.00 | 13.02 |
| ATOM | 8530 | CB | ARG | 1273 | −3.058 | −20.457 | 42.862 | 1.00 | 13.85 |
| ATOM | 8531 | CG | ARG | 1273 | −3.681 | −21.381 | 41.817 | 1.00 | 15.25 |
| ATOM | 8532 | CD | ARG | 1273 | −5.033 | −21.909 | 42.266 | 1.00 | 15.44 |
| ATOM | 8533 | NE | ARG | 1273 | −6.101 | −20.917 | 42.171 | 1.00 | 15.46 |
| ATOM | 8534 | CZ | ARG | 1273 | −7.292 | −21.060 | 42.742 | 1.00 | 16.08 |
| ATOM | 8535 | NH1 | ARG | 1273 | −7.558 | −22.151 | 43.454 | 1.00 | 14.02 |
| ATOM | 8536 | NH2 | ARG | 1273 | −8.218 | −20.120 | 42.592 | 1.00 | 14.57 |
| ATOM | 8537 | C | ARG | 1273 | −2.108 | −22.410 | 44.088 | 1.00 | 14.78 |
| ATOM | 8538 | O | ARG | 1273 | −2.710 | −23.466 | 43.933 | 1.00 | 15.68 |
| ATOM | 8539 | N | ARG | 1274 | −0.777 | −22.337 | 44.123 | 1.00 | 13.54 |
| ATOM | 8540 | CA | ARG | 1274 | 0.030 | −23.542 | 44.003 | 1.00 | 15.46 |
| ATOM | 8541 | CB | ARG | 1274 | 1.518 | −23.195 | 43.949 | 1.00 | 14.07 |
| ATOM | 8542 | CG | ARG | 1274 | 1.922 | −22.395 | 42.728 | 1.00 | 17.35 |
| ATOM | 8543 | CD | ARG | 1274 | 3.431 | −22.383 | 42.532 | 1.00 | 16.78 |
| ATOM | 8544 | NE | ARG | 1274 | 3.792 | −21.497 | 41.436 | 1.00 | 20.34 |
| ATOM | 8545 | CZ | ARG | 1274 | 4.066 | −20.203 | 41.573 | 1.00 | 20.54 |
| ATOM | 8546 | NH1 | ARG | 1274 | 4.038 | −19.634 | 42.772 | 1.00 | 18.45 |
| ATOM | 8547 | NH2 | ARG | 1274 | 4.341 | −19.468 | 40.502 | 1.00 | 22.65 |
| ATOM | 8548 | C | ARG | 1274 | −0.222 | −24.468 | 45.185 | 1.00 | 16.23 |
| ATOM | 8549 | O | ARG | 1274 | −0.225 | −25.693 | 45.043 | 1.00 | 16.53 |
| ATOM | 8550 | N | GLY | 1275 | −0.425 | −23.872 | 46.353 | 1.00 | 15.34 |
| ATOM | 8551 | CA | GLY | 1275 | −0.671 | −24.654 | 47.554 | 1.00 | 16.35 |
| ATOM | 8552 | C | GLY | 1275 | −2.105 | −25.138 | 47.686 | 1.00 | 17.09 |
| ATOM | 8553 | O | GLY | 1275 | −2.363 | −26.148 | 48.338 | 1.00 | 17.88 |
| ATOM | 8554 | N | ALA | 1276 | −3.042 | −24.417 | 47.077 | 1.00 | 15.42 |
| ATOM | 8555 | CA | ALA | 1276 | −4.457 | −24.785 | 47.141 | 1.00 | 15.88 |
| ATOM | 8556 | CB | ALA | 1276 | −5.158 | −23.921 | 48.170 | 1.00 | 12.41 |
| ATOM | 8557 | C | ALA | 1276 | −5.122 | −24.623 | 45.770 | 1.00 | 18.03 |
| ATOM | 8558 | O | ALA | 1276 | −5.898 | −23.690 | 45.546 | 1.00 | 17.23 |
| ATOM | 8559 | N | PRO | 1277 | −4.843 | −25.550 | 44.841 | 1.00 | 19.05 |
| ATOM | 8560 | CD | PRO | 1277 | −4.009 | −26.751 | 45.029 | 1.00 | 21.91 |
| ATOM | 8561 | CA | PRO | 1277 | −5.402 | −25.507 | 43.486 | 1.00 | 20.89 |
| ATOM | 8562 | CB | PRO | 1277 | −4.692 | −26.665 | 42.784 | 1.00 | 21.37 |
| ATOM | 8563 | CG | PRO | 1277 | −4.452 | −27.631 | 43.890 | 1.00 | 23.97 |
| ATOM | 8564 | C | PRO | 1277 | −6.922 | −25.574 | 43.340 | 1.00 | 21.51 |
| ATOM | 8565 | O | PRO | 1277 | −7.458 | −25.173 | 42.306 | 1.00 | 21.21 |
| ATOM | 8566 | N | ASN | 1278 | −7.615 | −26.049 | 44.371 | 1.00 | 20.14 |
| ATOM | 8567 | CA | ASN | 1278 | −9.065 | −26.171 | 44.297 | 1.00 | 21.69 |
| ATOM | 8568 | CB | ASN | 1278 | −9.483 | −27.622 | 44.567 | 1.00 | 25.95 |
| ATOM | 8569 | CG | ASN | 1278 | −8.833 | −28.600 | 43.610 | 1.00 | 26.75 |
| ATOM | 8570 | OD1 | ASN | 1278 | −8.927 | −28.446 | 42.393 | 1.00 | 30.57 |
| ATOM | 8571 | ND2 | ASN | 1278 | −8.173 | −29.612 | 44.154 | 1.00 | 29.13 |
| ATOM | 8572 | C | ASN | 1278 | −9.823 | −25.245 | 45.235 | 1.00 | 21.44 |
| ATOM | 8573 | O | ASN | 1278 | −11.042 | −25.346 | 45.364 | 1.00 | 20.63 |
| ATOM | 8574 | N | CYS | 1279 | −9.112 | −24.332 | 45.884 | 1.00 | 19.87 |
| ATOM | 8575 | CA | CYS | 1279 | −9.763 | −23.408 | 46.804 | 1.00 | 19.78 |
| ATOM | 8576 | CB | CYS | 1279 | −8.749 | −22.831 | 47.799 | 1.00 | 19.93 |
| ATOM | 8577 | SG | CYS | 1279 | −7.738 | −21.467 | 47.142 | 1.00 | 19.97 |
| ATOM | 8578 | C | CYS | 1279 | −10.401 | −22.245 | 46.055 | 1.00 | 19.66 |
| ATOM | 8579 | O | CYS | 1279 | −10.106 | −22.019 | 44.881 | 1.00 | 18.47 |
| ATOM | 8580 | N | LEU | 1280 | −11.294 | −21.530 | 46.738 | 1.00 | 18.36 |
| ATOM | 8581 | CA | LEU | 1280 | −11.924 | −20.336 | 46.179 | 1.00 | 17.84 |
| ATOM | 8582 | CB | LEU | 1280 | −13.296 | −20.059 | 46.814 | 1.00 | 19.49 |
| ATOM | 8583 | CG | LEU | 1280 | −13.960 | −18.734 | 46.406 | 1.00 | 18.08 |
| ATOM | 8584 | CD1 | LEU | 1280 | −14.186 | −18.712 | 44.897 | 1.00 | 19.62 |
| ATOM | 8585 | CD2 | LEU | 1280 | −15.291 | −18.559 | 47.142 | 1.00 | 17.89 |
| ATOM | 8586 | C | LEU | 1280 | −10.937 | −19.258 | 46.609 | 1.00 | 17.49 |
| ATOM | 8587 | O | LEU | 1280 | −10.763 | −19.003 | 47.805 | 1.00 | 16.91 |
| ATOM | 8588 | N | LEU | 1281 | −10.285 | −18.634 | 45.636 | 1.00 | 17.29 |
| ATOM | 8589 | CA | LEU | 1281 | −9.274 | −17.630 | 45.930 | 1.00 | 16.61 |
| ATOM | 8590 | CB | LEU | 1281 | −7.998 | −17.969 | 45.147 | 1.00 | 16.66 |
| ATOM | 8591 | CG | LEU | 1281 | −6.651 | −17.406 | 45.628 | 1.00 | 17.44 |
| ATOM | 8592 | CD1 | LEU | 1281 | −5.524 | −18.159 | 44.929 | 1.00 | 15.28 |
| ATOM | 8593 | CD2 | LEU | 1281 | −6.566 | −15.925 | 45.366 | 1.00 | 21.45 |
| ATOM | 8594 | C | LEU | 1281 | −9.692 | −16.195 | 45.633 | 1.00 | 17.29 |
| ATOM | 8595 | O | LEU | 1281 | −9.900 | −15.825 | 44.475 | 1.00 | 15.84 |
| ATOM | 8596 | N | LEU | 1282 | −9.820 | −15.398 | 46.689 | 1.00 | 16.41 |
| ATOM | 8597 | CA | LEU | 1282 | −10.168 | −13.983 | 46.561 | 1.00 | 17.05 |
| ATOM | 8598 | CB | LEU | 1282 | −11.093 | −13.534 | 47.699 | 1.00 | 16.06 |
| ATOM | 8599 | CG | LEU | 1282 | −12.601 | −13.795 | 47.550 | 1.00 | 17.50 |
| ATOM | 8600 | CD1 | LEU | 1282 | −12.847 | −15.285 | 47.468 | 1.00 | 17.75 |
| ATOM | 8601 | CD2 | LEU | 1282 | −13.358 | −13.176 | 48.732 | 1.00 | 18.80 |
| ATOM | 8602 | C | LEU | 1282 | −8.876 | −13.169 | 46.635 | 1.00 | 17.94 |
| ATOM | 8603 | O | LEU | 1282 | −8.030 | −13.425 | 47.487 | 1.00 | 22.05 |
| ATOM | 8604 | N | ALA | 1283 | −8.712 | −12.199 | 45.744 | 1.00 | 14.13 |
| ATOM | 8605 | CA | ALA | 1283 | −7.519 | −11.363 | 45.771 | 1.00 | 14.02 |
| ATOM | 8606 | CB | ALA | 1283 | −6.694 | −11.594 | 44.532 | 1.00 | 11.25 |
| ATOM | 8607 | C | ALA | 1283 | −7.940 | −9.903 | 45.855 | 1.00 | 12.74 |
| ATOM | 8608 | O | ALA | 1283 | −8.862 | −9.479 | 45.157 | 1.00 | 12.53 |
| ATOM | 8609 | N | ASP | 1284 | −7.287 | −9.131 | 46.719 | 1.00 | 13.81 |
| ATOM | 8610 | CA | ASP | 1284 | −7.623 | −7.718 | 46.827 | 1.00 | 14.31 |
| ATOM | 8611 | CB | ASP | 1284 | −7.075 | −7.095 | 48.113 | 1.00 | 17.63 |
| ATOM | 8612 | CG | ASP | 1284 | −7.972 | −7.298 | 49.310 | 1.00 | 19.26 |
| ATOM | 8613 | OD1 | ASP | 1284 | −9.202 | −7.418 | 49.152 | 1.00 | 17.82 |
| ATOM | 8614 | OD2 | ASP | 1284 | −7.428 | −7.301 | 50.430 | 1.00 | 23.10 |
| ATOM | 8615 | C | ASP | 1284 | −7.003 | −6.912 | 45.702 | 1.00 | 15.99 |
| ATOM | 8616 | O | ASP | 1284 | −5.930 | −7.256 | 45.202 | 1.00 | 14.33 |
| ATOM | 8617 | N | LEU | 1285 | −7.688 | −5.849 | 45.290 | 1.00 | 13.28 |
| ATOM | 8618 | CA | LEU | 1285 | −7.104 | −4.917 | 44.335 | 1.00 | 12.51 |
| ATOM | 8619 | CB | LEU | 1285 | −8.151 | −4.287 | 43.418 | 1.00 | 14.13 |
| ATOM | 8620 | CG | LEU | 1285 | −8.543 | −5.157 | 42.218 | 1.00 | 14.99 |
| ATOM | 8621 | CD1 | LEU | 1285 | −9.420 | −4.365 | 41.262 | 1.00 | 15.19 |
| ATOM | 8622 | CD2 | LEU | 1285 | −7.273 | −5.633 | 41.508 | 1.00 | 15.66 |
| ATOM | 8623 | C | LEU | 1285 | −6.616 | −3.907 | 45.373 | 1.00 | 13.60 |
| ATOM | 8624 | O | LEU | 1285 | −7.386 | −3.463 | 46.225 | 1.00 | 13.40 |
| ATOM | 8625 | N | PRO | 1286 | −5.319 | −3.569 | 45.349 | 1.00 | 13.01 |
| ATOM | 8626 | CD | PRO | 1286 | −4.276 | −4.134 | 44.477 | 1.00 | 12.67 |
| ATOM | 8627 | CA | PRO | 1286 | −4.751 | −2.621 | 46.312 | 1.00 | 13.67 |
| ATOM | 8628 | CB | PRO | 1286 | −3.250 | −2.909 | 46.223 | 1.00 | 14.83 |
| ATOM | 8629 | CG | PRO | 1286 | −3.075 | −3.237 | 44.758 | 1.00 | 13.00 |
| ATOM | 8630 | C | PRO | 1286 | −5.079 | −1.147 | 46.079 | 1.00 | 13.64 |
| ATOM | 8631 | O | PRO | 1286 | −5.772 | −0.781 | 45.128 | 1.00 | 14.01 |
| ATOM | 8632 | N | PHE | 1287 | −4.583 | −0.311 | 46.983 | 1.00 | 13.42 |
| ATOM | 8633 | CA | PHE | 1287 | −4.779 | 1.130 | 46.929 | 1.00 | 13.53 |
| ATOM | 8634 | CB | PHE | 1287 | −3.805 | 1.782 | 47.915 | 1.00 | 15.05 |
| ATOM | 8635 | CG | PHE | 1287 | −3.661 | 3.265 | 47.750 | 1.00 | 15.65 |
| ATOM | 8636 | CD1 | PHE | 1287 | −4.740 | 4.120 | 47.989 | 1.00 | 16.81 |
| ATOM | 8637 | CD2 | PHE | 1287 | −2.435 | 3.814 | 47.372 | 1.00 | 13.49 |
| ATOM | 8638 | CE1 | PHE | 1287 | −4.593 | 5.490 | 47.865 | 1.00 | 16.89 |
| ATOM | 8639 | CE2 | PHE | 1287 | −2.281 | 5.184 | 47.247 | 1.00 | 14.29 |
| ATOM | 8640 | CZ | PHE | 1287 | −3.367 | 6.032 | 47.492 | 1.00 | 17.50 |
| ATOM | 8641 | C | PHE | 1287 | −4.587 | 1.692 | 45.515 | 1.00 | 14.14 |
| ATOM | 8642 | O | PHE | 1287 | −3.581 | 1.418 | 44.851 | 1.00 | 11.80 |
| ATOM | 8643 | N | MET | 1288 | −5.562 | 2.484 | 45.071 | 1.00 | 13.14 |
| ATOM | 8644 | CA | MET | 1288 | −5.555 | 3.110 | 43.751 | 1.00 | 13.99 |
| ATOM | 8645 | CB | MET | 1288 | −4.470 | 4.198 | 43.687 | 1.00 | 15.56 |
| ATOM | 8646 | CG | MET | 1288 | −4.792 | 5.333 | 42.709 | 1.00 | 17.83 |
| ATOM | 8647 | SD | MET | 1288 | −6.295 | 6.296 | 43.134 | 1.00 | 16.61 |
| ATOM | 8648 | CE | MET | 1288 | −5.573 | 7.644 | 44.077 | 1.00 | 17.67 |
| ATOM | 8649 | C | MET | 1288 | −5.377 | 2.140 | 42.569 | 1.00 | 14.20 |
| ATOM | 8650 | O | MET | 1288 | −4.814 | 2.515 | 41.538 | 1.00 | 14.63 |
| ATOM | 8651 | N | ALA | 1289 | −5.840 | 0.901 | 42.704 | 1.00 | 12.49 |
| ATOM | 8652 | CA | ALA | 1289 | −5.723 | −0.046 | 41.596 | 1.00 | 13.64 |
| ATOM | 8653 | CB | ALA | 1289 | −5.342 | −1.438 | 42.112 | 1.00 | 12.98 |
| ATOM | 8654 | C | ALA | 1289 | −7.039 | −0.117 | 40.821 | 1.00 | 12.89 |
| ATOM | 8655 | O | ALA | 1289 | −7.142 | −0.819 | 39.816 | 1.00 | 13.36 |
| ATOM | 8656 | N | TYR | 1290 | −8.045 | 0.623 | 41.276 | 1.00 | 11.64 |
| ATOM | 8657 | CA | TYR | 1290 | −9.347 | 0.624 | 40.605 | 1.00 | 12.12 |
| ATOM | 8658 | CB | TYR | 1290 | −10.284 | −0.363 | 41.302 | 1.00 | 10.86 |
| ATOM | 8659 | CG | TYR | 1290 | −10.276 | −0.270 | 42.816 | 1.00 | 12.41 |
| ATOM | 8660 | CD1 | TYR | 1290 | −11.308 | 0.371 | 43.500 | 1.00 | 10.35 |
| ATOM | 8661 | CE1 | TYR | 1290 | −11.321 | 0.436 | 44.892 | 1.00 | 13.90 |
| ATOM | 8662 | CD2 | TYR | 1290 | −9.245 | −0.844 | 43.563 | 1.00 | 12.59 |
| ATOM | 8663 | CE2 | TYR | 1290 | −9.242 | −0.786 | 44.959 | 1.00 | 15.77 |
| ATOM | 8664 | CZ | TYR | 1290 | −10.290 | −0.144 | 45.616 | 1.00 | 16.49 |
| ATOM | 8665 | OH | TYR | 1290 | −10.313 | −0.095 | 46.992 | 1.00 | 16.97 |
| ATOM | 8666 | C | TYR | 1290 | −9.946 | 2.029 | 40.593 | 1.00 | 11.80 |
| ATOM | 8667 | O | TYR | 1290 | −11.142 | 2.212 | 40.811 | 1.00 | 12.18 |
| ATOM | 8668 | N | ALA | 1291 | −9.095 | 3.009 | 40.310 | 1.00 | 11.23 |
| ATOM | 8669 | CA | ALA | 1291 | −9.484 | 4.414 | 40.291 | 1.00 | 12.52 |
| ATOM | 8670 | CB | ALA | 1291 | −8.233 | 5.291 | 40.232 | 1.00 | 13.30 |
| ATOM | 8671 | C | ALA | 1291 | −10.425 | 4.748 | 39.141 | 1.00 | 13.87 |
| ATOM | 8672 | O | ALA | 1291 | −11.127 | 5.755 | 39.183 | 1.00 | 15.07 |
| ATOM | 8673 | N | THR | 1292 | −10.402 | 3.929 | 38.093 | 1.00 | 13.11 |
| ATOM | 8674 | CA | THR | 1292 | −11.295 | 4.111 | 36.953 | 1.00 | 13.77 |
| ATOM | 8675 | CB | THR | 1292 | −10.620 | 4.827 | 35.753 | 1.00 | 14.58 |
| ATOM | 8676 | OG1 | THR | 1292 | −9.685 | 3.944 | 35.123 | 1.00 | 13.37 |
| ATOM | 8677 | CG2 | THR | 1292 | −9.880 | 6.085 | 36.210 | 1.00 | 15.36 |
| ATOM | 8678 | C | THR | 1292 | −11.676 | 2.699 | 36.526 | 1.00 | 13.97 |
| ATOM | 8679 | O | THR | 1292 | −10.972 | 1.728 | 36.849 | 1.00 | 12.52 |
| ATOM | 8680 | N | PRO | 1293 | −12.805 | 2.554 | 35.821 | 1.00 | 12.10 |
| ATOM | 8681 | CD | PRO | 1293 | −13.854 | 3.557 | 35.555 | 1.00 | 11.83 |
| ATOM | 8682 | CA | PRO | 1293 | −13.228 | 1.222 | 35.376 | 1.00 | 12.00 |
| ATOM | 8683 | CB | PRO | 1293 | −14.503 | 1.517 | 34.593 | 1.00 | 11.85 |
| ATOM | 8684 | CG | PRO | 1293 | −15.078 | 2.695 | 35.378 | 1.00 | 11.15 |
| ATOM | 8685 | C | PRO | 1293 | −12.138 | 0.577 | 34.518 | 1.00 | 14.03 |
| ATOM | 8686 | O | PRO | 1293 | −11.789 | −0.588 | 34.712 | 1.00 | 15.96 |
| ATOM | 8687 | N | GLU | 1294 | −11.592 | 1.351 | 33.585 | 1.00 | 14.44 |
| ATOM | 8688 | CA | GLU | 1294 | −10.533 | 0.879 | 32.689 | 1.00 | 17.39 |
| ATOM | 8689 | CB | GLU | 1294 | −10.024 | 2.047 | 31.836 | 1.00 | 20.55 |
| ATOM | 8690 | CG | GLU | 1294 | −8.898 | 1.708 | 30.876 | 1.00 | 28.83 |
| ATOM | 8691 | CD | GLU | 1294 | −8.043 | 2.922 | 30.527 | 1.00 | 32.90 |
| ATOM | 8692 | OE1 | GLU | 1294 | −7.124 | 3.253 | 31.311 | 1.00 | 36.01 |
| ATOM | 8693 | OE2 | GLU | 1294 | −8.301 | 3.553 | 29.479 | 1.00 | 37.17 |
| ATOM | 8694 | C | GLU | 1294 | −9.366 | 0.279 | 33.471 | 1.00 | 16.65 |
| ATOM | 8695 | O | GLU | 1294 | −8.904 | −0.828 | 33.179 | 1.00 | 15.04 |
| ATOM | 8696 | N | GLN | 1295 | −8.873 | 1.011 | 34.462 | 1.00 | 15.47 |
| ATOM | 8697 | CA | GLN | 1295 | −7.760 | 0.500 | 35.257 | 1.00 | 15.54 |
| ATOM | 8698 | CB | GLN | 1295 | −7.184 | 1.609 | 36.133 | 1.00 | 16.24 |
| ATOM | 8699 | CG | GLN | 1295 | −6.427 | 2.633 | 35.322 | 1.00 | 23.28 |
| ATOM | 8700 | CD | GLN | 1295 | −6.020 | 3.825 | 36.139 | 1.00 | 24.86 |
| ATOM | 8701 | OE1 | GLN | 1295 | −5.407 | 3.679 | 37.192 | 1.00 | 24.58 |
| ATOM | 8702 | NE2 | GLN | 1295 | −6.356 | 5.021 | 35.656 | 1.00 | 26.69 |
| ATOM | 8703 | C | GLN | 1295 | −8.182 | −0.682 | 36.106 | 1.00 | 13.96 |
| ATOM | 8704 | O | GLN | 1295 | −7.394 | −1.606 | 36.338 | 1.00 | 13.72 |
| ATOM | 8705 | N | ALA | 1296 | −9.424 | −0.654 | 36.576 | 1.00 | 13.84 |
| ATOM | 8706 | CA | ALA | 1296 | −9.936 | −1.755 | 37.377 | 1.00 | 13.08 |
| ATOM | 8707 | CB | ALA | 1296 | −11.364 | −1.452 | 37.848 | 1.00 | 14.56 |
| ATOM | 8708 | C | ALA | 1296 | −9.925 | −3.030 | 36.528 | 1.00 | 12.59 |
| ATOM | 8709 | O | ALA | 1296 | −9.525 | −4.083 | 37.010 | 1.00 | 13.75 |
| ATOM | 8710 | N | PHE | 1297 | −10.345 | −2.934 | 35.265 | 1.00 | 10.91 |
| ATOM | 8711 | CA | PHE | 1297 | −10.363 | −4.121 | 34.399 | 1.00 | 11.05 |
| ATOM | 8712 | CB | PHE | 1297 | −10.886 | −3.819 | 32.985 | 1.00 | 9.65 |
| ATOM | 8713 | CG | PHE | 1297 | −12.206 | −3.090 | 32.942 | 1.00 | 10.56 |
| ATOM | 8714 | CD1 | PHE | 1297 | −13.170 | −3.279 | 33.929 | 1.00 | 10.04 |
| ATOM | 8715 | CD2 | PHE | 1297 | −12.475 | −2.205 | 31.898 | 1.00 | 11.57 |
| ATOM | 8716 | CE1 | PHE | 1297 | −14.387 | −2.593 | 33.891 | 1.00 | 10.32 |
| ATOM | 8717 | CE2 | PHE | 1297 | −13.684 | −1.513 | 31.840 | 1.00 | 13.36 |
| ATOM | 8718 | CZ | PHE | 1297 | −14.643 | −1.706 | 32.841 | 1.00 | 12.32 |
| ATOM | 8719 | C | PHE | 1297 | −8.956 | −4.698 | 34.240 | 1.00 | 10.73 |
| ATOM | 8720 | O | PHE | 1297 | −8.767 | −5.908 | 34.318 | 1.00 | 10.33 |
| ATOM | 8721 | N | GLU | 1298 | −7.981 | −3.827 | 33.998 | 1.00 | 10.95 |
| ATOM | 8722 | CA | GLU | 1298 | −6.607 | −4.265 | 33.799 | 1.00 | 12.53 |
| ATOM | 8723 | CB | GLU | 1298 | −5.736 | −3.091 | 33.353 | 1.00 | 16.83 |
| ATOM | 8724 | CG | GLU | 1298 | −4.361 | −3.499 | 32.848 | 1.00 | 24.35 |
| ATOM | 8725 | CD | GLU | 1298 | −4.377 | −3.908 | 31.382 | 1.00 | 28.25 |
| ATOM | 8726 | OE1 | GLU | 1298 | −5.089 | −4.875 | 31.025 | 1.00 | 30.56 |
| ATOM | 8727 | OE2 | GLU | 1298 | −3.681 | −3.248 | 30.576 | 1.00 | 33.57 |
| ATOM | 8728 | C | GLU | 1298 | −5.990 | −4.913 | 35.036 | 1.00 | 11.29 |
| ATOM | 8729 | O | GLU | 1298 | −5.396 | −5.989 | 34.951 | 1.00 | 10.65 |
| ATOM | 8730 | N | ASN | 1299 | −6.130 | −4.274 | 36.190 | 1.00 | 10.85 |
| ATOM | 8731 | CA | ASN | 1299 | −5.552 | −4.838 | 37.414 | 1.00 | 11.72 |
| ATOM | 8732 | CB | ASN | 1299 | −5.470 | −3.769 | 38.511 | 1.00 | 11.28 |
| ATOM | 8733 | CG | ASN | 1299 | −4.544 | −2.621 | 38.121 | 1.00 | 14.85 |
| ATOM | 8734 | OD1 | ASN | 1299 | −3.460 | −2.857 | 37.566 | 1.00 | 13.95 |
| ATOM | 8735 | ND2 | ASN | 1299 | −4.944 | −1.389 | 38.413 | 1.00 | 13.47 |
| ATOM | 8736 | C | ASN | 1299 | −6.301 | −6.070 | 37.905 | 1.00 | 11.79 |
| ATOM | 8737 | O | ASN | 1299 | −5.692 | −6.983 | 38.473 | 1.00 | 11.65 |
| ATOM | 8738 | N | ALA | 1300 | −7.619 | −6.104 | 37.701 | 1.00 | 11.62 |
| ATOM | 8739 | CA | ALA | 1300 | −8.384 | −7.281 | 38.095 | 1.00 | 9.62 |
| ATOM | 8740 | CB | ALA | 1300 | −9.864 | −7.042 | 37.911 | 1.00 | 11.93 |
| ATOM | 8741 | C | ALA | 1300 | −7.927 | −8.436 | 37.209 | 1.00 | 9.30 |
| ATOM | 8742 | O | ALA | 1300 | −7.725 | −9.552 | 37.688 | 1.00 | 11.63 |
| ATOM | 8743 | N | ALA | 1301 | −7.745 | −8.176 | 35.918 | 1.00 | 9.27 |
| ATOM | 8744 | CA | ALA | 1301 | −7.318 | −9.247 | 35.033 | 1.00 | 10.11 |
| ATOM | 8745 | CB | ALA | 1301 | −7.226 | −8.749 | 33.591 | 1.00 | 8.32 |
| ATOM | 8746 | C | ALA | 1301 | −5.981 | −9.807 | 35.492 | 1.00 | 10.42 |
| ATOM | 8747 | O | ALA | 1301 | −5.794 | −11.029 | 35.523 | 1.00 | 10.44 |
| ATOM | 8748 | N | THR | 1302 | −5.055 | −8.926 | 35.873 | 1.00 | 10.18 |
| ATOM | 8749 | CA | THR | 1302 | −3.729 | −9.378 | 36.312 | 1.00 | 10.80 |
| ATOM | 8750 | CB | THR | 1302 | −2.821 | −8.189 | 36.740 | 1.00 | 11.76 |
| ATOM | 8751 | OG1 | THR | 1302 | −2.586 | −7.327 | 35.612 | 1.00 | 12.59 |
| ATOM | 8752 | CG2 | THR | 1302 | −1.479 | −8.702 | 37.260 | 1.00 | 11.04 |
| ATOM | 8753 | C | THR | 1302 | −3.801 | −10.378 | 37.457 | 1.00 | 10.37 |
| ATOM | 8754 | O | THR | 1302 | −3.177 | −11.439 | 37.391 | 1.00 | 10.85 |
| ATOM | 8755 | N | VAL | 1303 | −4.563 | −10.070 | 38.502 | 1.00 | 9.66 |
| ATOM | 8756 | CA | VAL | 1303 | −4.622 | −10.991 | 39.630 | 1.00 | 9.50 |
| ATOM | 8757 | CB | VAL | 1303 | −5.112 | −10.277 | 40.894 | 1.00 | 13.99 |
| ATOM | 8758 | CG1 | VAL | 1303 | −4.904 | −11.178 | 42.085 | 1.00 | 18.44 |
| ATOM | 8759 | CG2 | VAL | 1303 | −4.326 | −8.981 | 41.091 | 1.00 | 11.75 |
| ATOM | 8760 | C | VAL | 1303 | −5.440 | −12.253 | 39.358 | 1.00 | 10.29 |
| ATOM | 8761 | O | VAL | 1303 | −5.187 | −13.312 | 39.955 | 1.00 | 8.82 |
| ATOM | 8762 | N | MET | 1304 | −6.412 | −12.148 | 38.453 | 1.00 | 10.19 |
| ATOM | 8763 | CA | MET | 1304 | −7.219 | −13.307 | 38.069 | 1.00 | 12.39 |
| ATOM | 8764 | CB | MET | 1304 | −8.431 | −12.884 | 37.221 | 1.00 | 13.83 |
| ATOM | 8765 | CG | MET | 1304 | −9.485 | −12.065 | 37.951 | 1.00 | 18.90 |
| ATOM | 8766 | SD | MET | 1304 | −10.483 | −13.022 | 39.099 | 1.00 | 21.49 |
| ATOM | 8767 | CE | MET | 1304 | −11.599 | −13.972 | 37.916 | 1.00 | 17.14 |
| ATOM | 8768 | C | MET | 1304 | −6.345 | −14.277 | 37.254 | 1.00 | 11.47 |
| ATOM | 8769 | O | MET | 1304 | −6.400 | −15.495 | 37.462 | 1.00 | 11.55 |
| ATOM | 8770 | N | ARG | 1305 | −5.549 | −13.746 | 36.323 | 1.00 | 11.47 |
| ATOM | 8771 | CA | ARG | 1305 | −4.674 | −14.603 | 35.514 | 1.00 | 9.90 |
| ATOM | 8772 | CB | ARG | 1305 | −3.925 | −13.805 | 34.441 | 1.00 | 9.95 |
| ATOM | 8773 | CG | ARG | 1305 | −4.805 | −13.184 | 33.373 | 1.00 | 13.47 |
| ATOM | 8774 | CD | ARG | 1305 | −4.001 | −12.850 | 32.131 | 1.00 | 13.07 |
| ATOM | 8775 | NE | ARG | 1305 | −4.782 | −12.012 | 31.231 | 1.00 | 18.31 |
| ATOM | 8776 | CZ | ARG | 1305 | −4.895 | −10.694 | 31.343 | 1.00 | 17.19 |
| ATOM | 8777 | NH1 | ARG | 1305 | −4.261 | −10.044 | 32.317 | 1.00 | 17.95 |
| ATOM | 8778 | NH2 | ARG | 1305 | −5.680 | −10.033 | 30.500 | 1.00 | 16.90 |
| ATOM | 8779 | C | ARG | 1305 | −3.643 | −15.280 | 36.411 | 1.00 | 11.06 |
| ATOM | 8780 | O | ARG | 1305 | −3.161 | −16.370 | 36.103 | 1.00 | 10.28 |
| ATOM | 8781 | N | ALA | 1306 | −3.314 | −14.624 | 37.519 | 1.00 | 9.99 |
| ATOM | 8782 | CA | ALA | 1306 | −2.342 | −15.166 | 38.449 | 1.00 | 11.44 |
| ATOM | 8783 | CB | ALA | 1306 | −1.767 | −14.061 | 39.321 | 1.00 | 10.39 |
| ATOM | 8784 | C | ALA | 1306 | −2.919 | −16.278 | 39.319 | 1.00 | 12.44 |
| ATOM | 8785 | O | ALA | 1306 | −2.172 | −16.960 | 40.013 | 1.00 | 12.32 |
| ATOM | 8786 | N | GLY | 1307 | −4.238 | −16.455 | 39.297 | 1.00 | 12.27 |
| ATOM | 8787 | CA | GLY | 1307 | −4.823 | −17.529 | 40.075 | 1.00 | 13.76 |
| ATOM | 8788 | C | GLY | 1307 | −6.098 | −17.245 | 40.850 | 1.00 | 13.42 |
| ATOM | 8789 | O | GLY | 1307 | −6.733 | −18.176 | 41.345 | 1.00 | 14.10 |
| ATOM | 8790 | N | ALA | 1308 | −6.481 | −15.979 | 40.964 | 1.00 | 11.75 |
| ATOM | 8791 | CA | ALA | 1308 | −7.690 | −15.619 | 41.706 | 1.00 | 12.70 |
| ATOM | 8792 | CB | ALA | 1308 | −7.653 | −14.144 | 42.065 | 1.00 | 12.41 |
| ATOM | 8793 | C | ALA | 1308 | −8.984 | −15.939 | 40.958 | 1.00 | 12.62 |
| ATOM | 8794 | O | ALA | 1308 | −9.007 | −15.996 | 39.725 | 1.00 | 12.19 |
| ATOM | 8795 | N | ASN | 1309 | −10.058 | −16.165 | 41.715 | 1.00 | 12.53 |
| ATOM | 8796 | CA | ASN | 1309 | −11.368 | −16.466 | 41.138 | 1.00 | 13.45 |
| ATOM | 8797 | CB | ASN | 1309 | −12.040 | −17.641 | 41.852 | 1.00 | 14.86 |
| ATOM | 8798 | CG | ASN | 1309 | −11.249 | −18.914 | 41.769 | 1.00 | 15.09 |
| ATOM | 8799 | OD1 | ASN | 1309 | −10.999 | −19.445 | 40.682 | 1.00 | 16.19 |
| ATOM | 8800 | ND2 | ASN | 1309 | −10.852 | −19.426 | 42.926 | 1.00 | 12.00 |
| ATOM | 8801 | C | ASN | 1309 | −12.288 | −15.271 | 41.323 | 1.00 | 13.77 |
| ATOM | 8802 | O | ASN | 1309 | −13.353 | −15.207 | 40.719 | 1.00 | 14.80 |
| ATOM | 8803 | N | MET | 1310 | −11.870 | −14.332 | 42.165 | 1.00 | 13.61 |
| ATOM | 8804 | CA | MET | 1310 | −12.687 | −13.168 | 42.491 | 1.00 | 12.31 |
| ATOM | 8805 | CB | MET | 1310 | −13.708 | −13.584 | 43.562 | 1.00 | 14.22 |
| ATOM | 8806 | CG | MET | 1310 | −14.634 | −12.499 | 44.092 | 1.00 | 17.85 |
| ATOM | 8807 | SD | MET | 1310 | −15.747 | −13.199 | 45.346 | 1.00 | 20.90 |
| ATOM | 8808 | CE | MET | 1310 | −17.324 | −13.078 | 44.544 | 1.00 | 22.97 |
| ATOM | 8809 | C | MET | 1310 | −11.794 | −12.051 | 43.016 | 1.00 | 12.24 |
| ATOM | 8810 | O | MET | 1310 | −10.737 | −12.304 | 43.589 | 1.00 | 11.09 |
| ATOM | 8811 | N | VAL | 1311 | −12.227 | −10.816 | 42.818 | 1.00 | 12.28 |
| ATOM | 8812 | CA | VAL | 1311 | −11.466 | −9.666 | 43.272 | 1.00 | 13.29 |
| ATOM | 8813 | CB | VAL | 1311 | −11.215 | −8.728 | 42.088 | 1.00 | 15.81 |
| ATOM | 8814 | CG1 | VAL | 1311 | −10.651 | −7.421 | 42.566 | 1.00 | 19.74 |
| ATOM | 8815 | CG2 | VAL | 1311 | −10.253 | −9.399 | 41.106 | 1.00 | 16.21 |
| ATOM | 8816 | C | VAL | 1311 | −12.234 | −8.923 | 44.360 | 1.00 | 13.00 |
| ATOM | 8817 | O | VAL | 1311 | −13.462 | −8.831 | 44.303 | 1.00 | 13.49 |
| ATOM | 8818 | N | LYS | 1312 | −11.519 | −8.409 | 45.355 | 1.00 | 12.33 |
| ATOM | 8819 | CA | LYS | 1312 | −12.158 | −7.642 | 46.413 | 1.00 | 11.16 |
| ATOM | 8820 | CB | LYS | 1312 | −11.806 | −8.197 | 47.797 | 1.00 | 13.10 |
| ATOM | 8821 | CG | LYS | 1312 | −12.465 | −7.402 | 48.925 | 1.00 | 15.32 |
| ATOM | 8822 | CD | LYS | 1312 | −12.623 | −8.211 | 50.207 | 1.00 | 16.26 |
| ATOM | 8823 | CE | LYS | 1312 | −11.297 | −8.393 | 50.927 | 1.00 | 18.97 |
| ATOM | 8824 | NZ | LYS | 1312 | −10.732 | −7.084 | 51.357 | 1.00 | 15.88 |
| ATOM | 8825 | C | LYS | 1312 | −11.716 | −6.188 | 46.315 | 1.00 | 13.64 |
| ATOM | 8826 | O | LYS | 1312 | −10.526 | −5.897 | 46.172 | 1.00 | 13.12 |
| ATOM | 8827 | N | ILE | 1313 | −12.680 | −5.274 | 46.381 | 1.00 | 12.62 |
| ATOM | 8828 | CA | ILE | 1313 | −12.388 | −3.842 | 46.308 | 1.00 | 14.45 |
| ATOM | 8829 | CB | ILE | 1313 | −12.802 | −3.250 | 44.949 | 1.00 | 16.94 |
| ATOM | 8830 | CG2 | ILE | 1313 | −11.890 | −3.779 | 43.839 | 1.00 | 19.34 |
| ATOM | 8831 | CG1 | ILE | 1313 | −14.266 | −3.588 | 44.661 | 1.00 | 18.39 |
| ATOM | 8832 | CD1 | ILE | 1313 | −14.764 | −3.036 | 43.343 | 1.00 | 19.90 |
| ATOM | 8833 | C | ILE | 1313 | −13.127 | −3.086 | 47.413 | 1.00 | 14.85 |
| ATOM | 8834 | O | ILE | 1313 | −14.264 | −3.423 | 47.748 | 1.00 | 13.87 |
| ATOM | 8835 | N | GLU | 1314 | −12.483 | −2.054 | 47.951 | 1.00 | 15.54 |
| ATOM | 8836 | CA | GLU | 1314 | −13.047 | −1.259 | 49.043 | 1.00 | 14.90 |
| ATOM | 8837 | CB | GLU | 1314 | −11.944 | −0.825 | 50.019 | 1.00 | 16.10 |
| ATOM | 8838 | CG | GLU | 1314 | −11.092 | −1.946 | 50.573 | 1.00 | 17.92 |
| ATOM | 8839 | CD | GLU | 1314 | −9.990 | −1.445 | 51.512 | 1.00 | 18.81 |
| ATOM | 8840 | OE1 | GLU | 1314 | −9.869 | −0.218 | 51.713 | 1.00 | 18.97 |
| ATOM | 8841 | OE2 | GLU | 1314 | −9.250 | −2.287 | 52.056 | 1.00 | 20.27 |
| ATOM | 8842 | C | GLU | 1314 | −13.783 | −0.009 | 48.587 | 1.00 | 14.25 |
| ATOM | 8843 | O | GLU | 1314 | −13.290 | 0.737 | 47.746 | 1.00 | 16.21 |
| ATOM | 8844 | N | GLY | 1315 | −14.963 | 0.229 | 49.151 | 1.00 | 16.28 |
| ATOM | 8845 | CA | GLY | 1315 | −15.705 | 1.416 | 48.781 | 1.00 | 16.32 |
| ATOM | 8846 | C | GLY | 1315 | −17.196 | 1.197 | 48.692 | 1.00 | 16.99 |
| ATOM | 8847 | O | GLY | 1315 | −17.662 | 0.060 | 48.630 | 1.00 | 16.14 |
| ATOM | 8848 | N | GLY | 1316 | −17.942 | 2.295 | 48.669 | 1.00 | 16.84 |
| ATOM | 8849 | CA | GLY | 1316 | −19.390 | 2.207 | 48.600 | 1.00 | 18.58 |
| ATOM | 8850 | C | GLY | 1316 | −19.974 | 2.452 | 47.224 | 1.00 | 19.09 |
| ATOM | 8851 | O | GLY | 1316 | −19.491 | 1.928 | 46.222 | 1.00 | 18.98 |
| ATOM | 8852 | N | GLU | 1317 | −21.022 | 3.268 | 47.196 | 1.00 | 19.78 |
| ATOM | 8853 | CA | GLU | 1317 | −21.753 | 3.608 | 45.983 | 1.00 | 21.03 |
| ATOM | 8854 | CB | GLU | 1317 | −22.791 | 4.686 | 46.302 | 1.00 | 24.13 |
| ATOM | 8855 | CG | GLU | 1317 | −24.226 | 4.206 | 46.235 | 1.00 | 34.04 |
| ATOM | 8856 | CD | GLU | 1317 | −24.910 | 4.591 | 44.946 | 1.00 | 35.84 |
| ATOM | 8857 | OE1 | GLU | 1317 | −24.373 | 4.264 | 43.863 | 1.00 | 40.02 |
| ATOM | 8858 | OE2 | GLU | 1317 | −25.986 | 5.219 | 45.018 | 1.00 | 38.96 |
| ATOM | 8859 | C | GLU | 1317 | −20.948 | 4.052 | 44.768 | 1.00 | 19.20 |
| ATOM | 8860 | O | GLU | 1317 | −21.318 | 3.733 | 43.640 | 1.00 | 17.96 |
| ATOM | 8861 | N | TRP | 1318 | −19.857 | 4.783 | 44.981 | 1.00 | 17.44 |
| ATOM | 8862 | CA | TRP | 1318 | −19.079 | 5.258 | 43.846 | 1.00 | 17.52 |
| ATOM | 8863 | CB | TRP | 1318 | −17.909 | 6.149 | 44.301 | 1.00 | 18.44 |
| ATOM | 8864 | CG | TRP | 1318 | −16.754 | 5.430 | 44.951 | 1.00 | 17.60 |
| ATOM | 8865 | CD2 | TRP | 1318 | −15.534 | 5.018 | 44.316 | 1.00 | 17.10 |
| ATOM | 8866 | CE2 | TRP | 1318 | −14.730 | 4.408 | 45.307 | 1.00 | 16.85 |
| ATOM | 8867 | CE3 | TRP | 1318 | −15.043 | 5.106 | 43.005 | 1.00 | 15.81 |
| ATOM | 8868 | CD1 | TRP | 1318 | −16.645 | 5.060 | 46.262 | 1.00 | 18.81 |
| ATOM | 8869 | NE1 | TRP | 1318 | −15.431 | 4.448 | 46.483 | 1.00 | 17.06 |
| ATOM | 8870 | CZ2 | TRP | 1318 | −13.458 | 3.888 | 45.030 | 1.00 | 16.56 |
| ATOM | 8871 | CZ3 | TRP | 1318 | −13.774 | 4.588 | 42.727 | 1.00 | 16.65 |
| ATOM | 8872 | CH2 | TRP | 1318 | −13.000 | 3.987 | 43.736 | 1.00 | 13.94 |
| ATOM | 8873 | C | TRP | 1318 | −18.557 | 4.121 | 42.972 | 1.00 | 15.10 |
| ATOM | 8874 | O | TRP | 1318 | −18.168 | 4.346 | 41.830 | 1.00 | 17.22 |
| ATOM | 8875 | N | LEU | 1319 | −18.580 | 2.903 | 43.507 | 1.00 | 15.72 |
| ATOM | 8876 | CA | LEU | 1319 | −18.100 | 1.712 | 42.804 | 1.00 | 14.82 |
| ATOM | 8877 | CB | LEU | 1319 | −17.556 | 0.698 | 43.813 | 1.00 | 15.07 |
| ATOM | 8878 | CG | LEU | 1319 | −16.228 | 1.040 | 44.490 | 1.00 | 15.24 |
| ATOM | 8879 | CD1 | LEU | 1319 | −15.872 | −0.036 | 45.504 | 1.00 | 14.37 |
| ATOM | 8880 | CD2 | LEU | 1319 | −15.139 | 1.148 | 43.429 | 1.00 | 14.65 |
| ATOM | 8881 | C | LEU | 1319 | −19.129 | 0.992 | 41.938 | 1.00 | 15.55 |
| ATOM | 8882 | O | LEU | 1319 | −18.779 | 0.080 | 41.192 | 1.00 | 16.40 |
| ATOM | 8883 | N | VAL | 1320 | −20.393 | 1.385 | 42.028 | 1.00 | 15.54 |
| ATOM | 8884 | CA | VAL | 1320 | −21.446 | 0.700 | 41.274 | 1.00 | 15.61 |
| ATOM | 8885 | CB | VAL | 1320 | −22.769 | 1.480 | 41.328 | 1.00 | 15.50 |
| ATOM | 8886 | CG1 | VAL | 1320 | −23.760 | 0.896 | 40.322 | 1.00 | 16.93 |
| ATOM | 8887 | CG2 | VAL | 1320 | −23.339 | 1.407 | 42.722 | 1.00 | 15.37 |
| ATOM | 8888 | C | VAL | 1320 | −21.114 | 0.401 | 39.819 | 1.00 | 15.88 |
| ATOM | 8889 | O | VAL | 1320 | −21.237 | −0.740 | 39.375 | 1.00 | 16.33 |
| ATOM | 8890 | N | GLU | 1321 | −20.703 | 1.427 | 39.078 | 1.00 | 15.89 |
| ATOM | 8891 | CA | GLU | 1321 | −20.367 | 1.256 | 37.667 | 1.00 | 16.86 |
| ATOM | 8892 | CB | GLU | 1321 | −19.982 | 2.601 | 37.038 | 1.00 | 18.42 |
| ATOM | 8893 | CG | GLU | 1321 | −19.483 | 2.486 | 35.604 | 1.00 | 23.16 |
| ATOM | 8894 | CD | GLU | 1321 | −19.156 | 3.836 | 34.967 | 1.00 | 27.92 |
| ATOM | 8895 | OE1 | GLU | 1321 | −18.295 | 4.576 | 35.495 | 1.00 | 28.97 |
| ATOM | 8896 | OE2 | GLU | 1321 | −19.761 | 4.153 | 33.923 | 1.00 | 30.95 |
| ATOM | 8897 | C | GLU | 1321 | −19.212 | 0.265 | 37.509 | 1.00 | 16.05 |
| ATOM | 8898 | O | GLU | 1321 | −19.258 | −0.639 | 36.671 | 1.00 | 13.68 |
| ATOM | 8899 | N | THR | 1322 | −18.180 | 0.441 | 38.325 | 1.00 | 13.47 |
| ATOM | 8900 | CA | THR | 1322 | −17.013 | −0.432 | 38.271 | 1.00 | 14.01 |
| ATOM | 8901 | CB | THR | 1322 | −15.983 | −0.025 | 39.352 | 1.00 | 14.03 |
| ATOM | 8902 | OG1 | THR | 1322 | −15.481 | 1.280 | 39.046 | 1.00 | 16.65 |
| ATOM | 8903 | CG2 | THR | 1322 | −14.807 | −1.001 | 39.395 | 1.00 | 13.60 |
| ATOM | 8904 | C | THR | 1322 | −17.432 | −1.887 | 38.462 | 1.00 | 11.93 |
| ATOM | 8905 | O | THR | 1322 | −17.064 | −2.768 | 37.675 | 1.00 | 13.98 |
| ATOM | 8906 | N | VAL | 1323 | −18.221 | −2.133 | 39.501 | 1.00 | 12.13 |
| ATOM | 8907 | CA | VAL | 1323 | −18.696 | −3.483 | 39.794 | 1.00 | 12.28 |
| ATOM | 8908 | CB | VAL | 1323 | −19.511 | −3.508 | 41.107 | 1.00 | 14.17 |
| ATOM | 8909 | CG1 | VAL | 1323 | −20.152 | −4.881 | 41.303 | 1.00 | 14.33 |
| ATOM | 8910 | CG2 | VAL | 1323 | −18.597 | −3.177 | 42.275 | 1.00 | 12.26 |
| ATOM | 8911 | C | VAL | 1323 | −19.537 | −4.061 | 38.647 | 1.00 | 13.33 |
| ATOM | 8912 | O | VAL | 1323 | −19.326 | −5.203 | 38.226 | 1.00 | 13.70 |
| ATOM | 8913 | N | GLN | 1324 | −20.480 | −3.274 | 38.137 | 1.00 | 13.37 |
| ATOM | 8914 | CA | GLN | 1324 | −21.322 | −3.740 | 37.039 | 1.00 | 15.20 |
| ATOM | 8915 | CB | GLN | 1324 | −22.300 | −2.640 | 36.599 | 1.00 | 16.72 |
| ATOM | 8916 | CG | GLN | 1324 | −23.292 | −2.224 | 37.682 | 1.00 | 22.21 |
| ATOM | 8917 | CD | GLN | 1324 | −24.211 | −1.098 | 37.244 | 1.00 | 24.87 |
| ATOM | 8918 | OE1 | GLN | 1324 | −23.764 | −0.121 | 36.646 | 1.00 | 26.46 |
| ATOM | 8919 | NE2 | GLN | 1324 | −25.498 | −1.222 | 37.552 | 1.00 | 27.32 |
| ATOM | 8920 | C | GLN | 1324 | −20.460 | −4.149 | 35.850 | 1.00 | 15.72 |
| ATOM | 8921 | O | GLN | 1324 | −20.617 | −5.237 | 35.304 | 1.00 | 14.44 |
| ATOM | 8922 | N | MET | 1325 | −19.536 | −3.273 | 35.469 | 1.00 | 14.94 |
| ATOM | 8923 | CA | MET | 1325 | −18.670 | −3.533 | 34.334 | 1.00 | 14.16 |
| ATOM | 8924 | CB | MET | 1325 | −17.942 | −2.251 | 33.932 | 1.00 | 13.53 |
| ATOM | 8925 | CG | MET | 1325 | −18.877 | −1.166 | 33.384 | 1.00 | 18.17 |
| ATOM | 8926 | SD | MET | 1325 | −17.961 | 0.297 | 32.829 | 1.00 | 18.88 |
| ATOM | 8927 | CE | MET | 1325 | −17.729 | −0.079 | 31.088 | 1.00 | 21.14 |
| ATOM | 8928 | C | MET | 1325 | −17.688 | −4.684 | 34.558 | 1.00 | 13.07 |
| ATOM | 8929 | O | MET | 1325 | −17.438 | −5.465 | 33.639 | 1.00 | 12.98 |
| ATOM | 8930 | N | LEU | 1326 | −17.127 | −4.809 | 35.757 | 1.00 | 12.81 |
| ATOM | 8931 | CA | LEU | 1326 | −16.213 | −5.919 | 36.009 | 1.00 | 13.89 |
| ATOM | 8932 | CB | LEU | 1326 | −15.616 | −5.835 | 37.417 | 1.00 | 13.67 |
| ATOM | 8933 | CG | LEU | 1326 | −14.431 | −4.878 | 37.574 | 1.00 | 14.23 |
| ATOM | 8934 | CD1 | LEU | 1326 | −14.102 | −4.695 | 39.044 | 1.00 | 13.52 |
| ATOM | 8935 | CD2 | LEU | 1326 | −13.228 | −5.438 | 36.815 | 1.00 | 13.49 |
| ATOM | 8936 | C | LEU | 1326 | −16.965 | −7.235 | 35.864 | 1.00 | 15.09 |
| ATOM | 8937 | O | LEU | 1326 | −16.477 | −8.177 | 35.229 | 1.00 | 17.34 |
| ATOM | 8938 | N | THR | 1327 | −18.159 | −7.292 | 36.453 | 1.00 | 15.39 |
| ATOM | 8939 | CA | THR | 1327 | −18.993 | −8.495 | 36.420 | 1.00 | 17.21 |
| ATOM | 8940 | CB | THR | 1327 | −20.327 | −8.284 | 37.203 | 1.00 | 19.12 |
| ATOM | 8941 | OG1 | THR | 1327 | −20.041 | −8.025 | 38.585 | 1.00 | 22.00 |
| ATOM | 8942 | CG2 | THR | 1327 | −21.203 | −9.530 | 37.120 | 1.00 | 21.12 |
| ATOM | 8943 | C | THR | 1327 | −19.331 | −8.944 | 34.999 | 1.00 | 17.87 |
| ATOM | 8944 | O | THR | 1327 | −19.200 | −10.123 | 34.673 | 1.00 | 15.79 |
| ATOM | 8945 | N | GLU | 1328 | −19.763 | −8.018 | 34.146 | 1.00 | 19.35 |
| ATOM | 8946 | CA | GLU | 1328 | −20.110 | −8.417 | 32.787 | 1.00 | 19.92 |
| ATOM | 8947 | CB | GLU | 1328 | −20.951 | −7.330 | 32.104 | 1.00 | 24.09 |
| ATOM | 8948 | CG | GLU | 1328 | −20.189 | −6.213 | 31.461 | 1.00 | 25.04 |
| ATOM | 8949 | CD | GLU | 1328 | −21.115 | −5.173 | 30.854 | 1.00 | 23.39 |
| ATOM | 8950 | OE1 | GLU | 1328 | −22.172 | −5.550 | 30.306 | 1.00 | 27.43 |
| ATOM | 8951 | OE2 | GLU | 1328 | −20.782 | −3.977 | 30.916 | 1.00 | 21.37 |
| ATOM | 8952 | C | GLU | 1328 | −18.861 | −8.769 | 31.971 | 1.00 | 20.62 |
| ATOM | 8953 | O | GLU | 1328 | −18.953 | −9.345 | 30.882 | 1.00 | 22.88 |
| ATOM | 8954 | N | ARG | 1329 | −17.692 | −8.443 | 32.516 | 1.00 | 18.60 |
| ATOM | 8955 | CA | ARG | 1329 | −16.425 | −8.760 | 31.870 | 1.00 | 16.07 |
| ATOM | 8956 | CB | ARG | 1329 | −15.499 | −7.539 | 31.903 | 1.00 | 15.66 |
| ATOM | 8957 | CG | ARG | 1329 | −15.916 | −6.473 | 30.899 | 1.00 | 12.22 |
| ATOM | 8958 | CD | ARG | 1329 | −15.278 | −5.098 | 31.121 | 1.00 | 15.51 |
| ATOM | 8959 | NE | ARG | 1329 | −15.805 | −4.147 | 30.144 | 1.00 | 11.85 |
| ATOM | 8960 | CZ | ARG | 1329 | −17.075 | −3.748 | 30.096 | 1.00 | 13.40 |
| ATOM | 8961 | NH1 | ARG | 1329 | −17.947 | −4.206 | 30.980 | 1.00 | 13.96 |
| ATOM | 8962 | NH2 | ARG | 1329 | −17.486 | −2.922 | 29.142 | 1.00 | 11.23 |
| ATOM | 8963 | C | ARG | 1329 | −15.737 | −9.986 | 32.496 | 1.00 | 16.04 |
| ATOM | 8964 | O | ARG | 1329 | −14.508 | −10.111 | 32.466 | 1.00 | 15.96 |
| ATOM | 8965 | N | ALA | 1330 | −16.541 | −10.872 | 33.078 | 1.00 | 15.89 |
| ATOM | 8966 | CA | ALA | 1330 | −16.060 | −12.129 | 33.678 | 1.00 | 14.56 |
| ATOM | 8967 | CB | ALA | 1330 | −15.213 | −12.897 | 32.650 | 1.00 | 14.48 |
| ATOM | 8968 | C | ALA | 1330 | −15.306 | −12.070 | 35.004 | 1.00 | 14.53 |
| ATOM | 8969 | O | ALA | 1330 | −14.685 | −13.053 | 35.409 | 1.00 | 14.82 |
| ATOM | 8970 | N | VAL | 1331 | −15.359 | −10.929 | 35.679 | 1.00 | 13.56 |
| ATOM | 8971 | CA | VAL | 1331 | −14.678 | −10.779 | 36.951 | 1.00 | 14.03 |
| ATOM | 8972 | CB | VAL | 1331 | −13.755 | −9.536 | 36.926 | 1.00 | 14.54 |
| ATOM | 8973 | CG1 | VAL | 1331 | −13.126 | −9.306 | 38.300 | 1.00 | 15.64 |
| ATOM | 8974 | CG2 | VAL | 1331 | −12.653 | −9.736 | 35.888 | 1.00 | 17.69 |
| ATOM | 8975 | C | VAL | 1331 | −15.667 | −10.663 | 38.117 | 1.00 | 13.93 |
| ATOM | 8976 | O | VAL | 1331 | −16.349 | −9.660 | 38.260 | 1.00 | 15.56 |
| ATOM | 8977 | N | PRO | 1332 | −15.779 | −11.716 | 38.946 | 1.00 | 14.57 |
| ATOM | 8978 | CD | PRO | 1332 | −15.253 | −13.080 | 38.790 | 1.00 | 12.80 |
| ATOM | 8979 | CA | PRO | 1332 | −16.706 | −11.638 | 40.079 | 1.00 | 14.22 |
| ATOM | 8980 | CB | PRO | 1332 | −16.793 | −13.077 | 40.572 | 1.00 | 15.61 |
| ATOM | 8981 | CG | PRO | 1332 | −15.500 | −13.663 | 40.155 | 1.00 | 20.45 |
| ATOM | 8982 | C | PRO | 1332 | −16.111 | −10.694 | 41.100 | 1.00 | 13.84 |
| ATOM | 8983 | O | PRO | 1332 | −14.888 | −10.616 | 41.268 | 1.00 | 11.75 |
| ATOM | 8984 | N | VAL | 1333 | −16.976 | −9.966 | 41.784 | 1.00 | 12.32 |
| ATOM | 8985 | CA | VAL | 1333 | −16.502 | −8.991 | 42.752 | 1.00 | 11.45 |
| ATOM | 8986 | CB | VAL | 1333 | −16.886 | −7.561 | 42.311 | 1.00 | 10.56 |
| ATOM | 8987 | CG1 | VAL | 1333 | −16.465 | −6.551 | 43.378 | 1.00 | 12.68 |
| ATOM | 8988 | CG2 | VAL | 1333 | −16.228 | −7.235 | 40.987 | 1.00 | 9.00 |
| ATOM | 8989 | C | VAL | 1333 | −17.022 | −9.206 | 44.150 | 1.00 | 13.49 |
| ATOM | 8990 | O | VAL | 1333 | −18.176 | −9.581 | 44.346 | 1.00 | 13.53 |
| ATOM | 8991 | N | CYS | 1334 | −16.145 | −8.987 | 45.122 | 1.00 | 12.49 |
| ATOM | 8992 | CA | CYS | 1334 | −16.515 | −9.072 | 46.524 | 1.00 | 13.08 |
| ATOM | 8993 | CB | CYS | 1334 | −15.561 | −9.988 | 47.294 | 1.00 | 14.71 |
| ATOM | 8994 | SG | CYS | 1334 | −15.827 | −9.940 | 49.091 | 1.00 | 15.45 |
| ATOM | 8995 | C | CYS | 1334 | −16.359 | −7.637 | 47.015 | 1.00 | 13.95 |
| ATOM | 8996 | O | CYS | 1334 | −15.312 | −7.011 | 46.818 | 1.00 | 14.76 |
| ATOM | 8997 | N | GLY | 1335 | −17.413 | −7.114 | 47.627 | 1.00 | 14.01 |
| ATOM | 8998 | CA | GLY | 1335 | −17.373 | −5.764 | 48.141 | 1.00 | 13.56 |
| ATOM | 8999 | C | GLY | 1335 | −16.726 | −5.747 | 49.510 | 1.00 | 13.93 |
| ATOM | 9000 | O | GLY | 1335 | −16.501 | −6.802 | 50.105 | 1.00 | 14.54 |
| ATOM | 9001 | N | HIS | 1336 | −16.445 | −4.551 | 50.017 | 1.00 | 14.71 |
| ATOM | 9002 | CA | HIS | 1336 | −15.801 | −4.397 | 51.320 | 1.00 | 14.53 |
| ATOM | 9003 | CB | HIS | 1336 | −14.277 | −4.469 | 51.143 | 1.00 | 13.80 |
| ATOM | 9004 | CG | HIS | 1336 | −13.511 | −4.501 | 52.431 | 1.00 | 16.43 |
| ATOM | 9005 | CD2 | HIS | 1336 | −13.866 | −4.166 | 53.697 | 1.00 | 14.92 |
| ATOM | 9006 | ND1 | HIS | 1336 | −12.199 | −4.917 | 52.502 | 1.00 | 16.33 |
| ATOM | 9007 | CE1 | HIS | 1336 | −11.778 | −4.839 | 53.752 | 1.00 | 17.68 |
| ATOM | 9008 | NE2 | HIS | 1336 | −12.769 | −4.386 | 54.498 | 1.00 | 15.76 |
| ATOM | 9009 | C | HIS | 1336 | −16.206 | −3.043 | 51.905 | 1.00 | 14.17 |
| ATOM | 9010 | O | HIS | 1336 | −15.852 | −2.005 | 51.363 | 1.00 | 13.31 |
| ATOM | 9011 | N | LEU | 1337 | −16.947 | −3.069 | 53.010 | 1.00 | 16.38 |
| ATOM | 9012 | CA | LEU | 1337 | −17.417 | −1.855 | 53.662 | 1.00 | 14.42 |
| ATOM | 9013 | CB | LEU | 1337 | −18.937 | −1.727 | 53.509 | 1.00 | 14.97 |
| ATOM | 9014 | CG | LEU | 1337 | −19.500 | −1.425 | 52.118 | 1.00 | 13.18 |
| ATOM | 9015 | CD1 | LEU | 1337 | −21.022 | −1.498 | 52.174 | 1.00 | 16.54 |
| ATOM | 9016 | CD2 | LEU | 1337 | −19.039 | −0.057 | 51.659 | 1.00 | 15.16 |
| ATOM | 9017 | C | LEU | 1337 | −17.074 | −1.819 | 55.152 | 1.00 | 15.08 |
| ATOM | 9018 | O | LEU | 1337 | −16.704 | −2.835 | 55.735 | 1.00 | 15.49 |
| ATOM | 9019 | N | GLY | 1338 | −17.234 | −0.646 | 55.763 | 1.00 | 16.52 |
| ATOM | 9020 | CA | GLY | 1338 | −16.931 | −0.487 | 57.175 | 1.00 | 18.65 |
| ATOM | 9021 | C | GLY | 1338 | −15.546 | 0.103 | 57.329 | 1.00 | 20.37 |
| ATOM | 9022 | O | GLY | 1338 | −15.244 | 1.169 | 56.779 | 1.00 | 20.72 |
| ATOM | 9023 | N | LEU | 1339 | −14.692 | −0.592 | 58.071 | 1.00 | 21.08 |
| ATOM | 9024 | CA | LEU | 1339 | −13.328 | −0.130 | 58.274 | 1.00 | 21.80 |
| ATOM | 9025 | CB | LEU | 1339 | −12.760 | −0.690 | 59.586 | 1.00 | 22.96 |
| ATOM | 9026 | CG | LEU | 1339 | −11.636 | 0.099 | 60.273 | 1.00 | 26.11 |
| ATOM | 9027 | CD1 | LEU | 1339 | −11.099 | −0.714 | 61.435 | 1.00 | 25.23 |
| ATOM | 9028 | CD2 | LEU | 1339 | −10.528 | 0.410 | 59.290 | 1.00 | 25.89 |
| ATOM | 9029 | C | LEU | 1339 | −12.500 | −0.633 | 57.096 | 1.00 | 21.63 |
| ATOM | 9030 | O | LEU | 1339 | −12.007 | −1.762 | 57.108 | 1.00 | 21.30 |
| ATOM | 9031 | N | THR | 1340 | −12.386 | 0.205 | 56.070 | 1.00 | 21.68 |
| ATOM | 9032 | CA | THR | 1340 | −11.618 | −0.104 | 54.864 | 1.00 | 22.62 |
| ATOM | 9033 | CB | THR | 1340 | −12.301 | 0.507 | 53.626 | 1.00 | 23.97 |
| ATOM | 9034 | OG1 | THR | 1340 | −12.526 | 1.906 | 53.842 | 1.00 | 23.53 |
| ATOM | 9035 | CG2 | THR | 1340 | −13.641 | −0.169 | 53.375 | 1.00 | 26.26 |
| ATOM | 9036 | C | THR | 1340 | −10.232 | 0.510 | 55.053 | 1.00 | 20.26 |
| ATOM | 9037 | O | THR | 1340 | −10.057 | 1.715 | 54.899 | 1.00 | 23.50 |
| ATOM | 9038 | N | PRO | 1341 | −9.226 | −0.323 | 55.372 | 1.00 | 20.57 |
| ATOM | 9039 | CD | PRO | 1341 | −9.300 | −1.794 | 55.327 | 1.00 | 21.40 |
| ATOM | 9040 | CA | PRO | 1341 | −7.842 | 0.114 | 55.603 | 1.00 | 19.00 |
| ATOM | 9041 | CB | PRO | 1341 | −7.091 | −1.205 | 55.802 | 1.00 | 22.07 |
| ATOM | 9042 | CG | PRO | 1341 | −7.881 | −2.171 | 54.978 | 1.00 | 25.73 |
| ATOM | 9043 | C | PRO | 1341 | −7.180 | 1.019 | 54.570 | 1.00 | 18.31 |
| ATOM | 9044 | O | PRO | 1341 | −6.332 | 1.832 | 54.928 | 1.00 | 16.70 |
| ATOM | 9045 | N | GLN | 1342 | −7.551 | 0.895 | 53.299 | 1.00 | 15.90 |
| ATOM | 9046 | CA | GLN | 1342 | −6.944 | 1.751 | 52.284 | 1.00 | 18.99 |
| ATOM | 9047 | CB | GLN | 1342 | −7.432 | 1.355 | 50.882 | 1.00 | 16.29 |
| ATOM | 9048 | CG | GLN | 1342 | −6.722 | 0.124 | 50.316 | 1.00 | 17.70 |
| ATOM | 9049 | CD | GLN | 1342 | −7.365 | −0.407 | 49.047 | 1.00 | 18.63 |
| ATOM | 9050 | OE1 | GLN | 1342 | −7.891 | 0.360 | 48.236 | 1.00 | 21.48 |
| ATOM | 9051 | NE2 | GLN | 1342 | −7.310 | −1.725 | 48.858 | 1.00 | 20.30 |
| ATOM | 9052 | C | GLN | 1342 | −7.225 | 3.235 | 52.559 | 1.00 | 18.46 |
| ATOM | 9053 | O | GLN | 1342 | −6.435 | 4.104 | 52.189 | 1.00 | 20.27 |
| ATOM | 9054 | N | SER | 1343 | −8.347 | 3.528 | 53.207 | 1.00 | 20.86 |
| ATOM | 9055 | CA | SER | 1343 | −8.684 | 4.913 | 53.528 | 1.00 | 21.17 |
| ATOM | 9056 | CB | SER | 1343 | −10.190 | 5.154 | 53.330 | 1.00 | 22.94 |
| ATOM | 9057 | OG | SER | 1343 | −10.574 | 4.937 | 51.979 | 1.00 | 24.54 |
| ATOM | 9058 | C | SER | 1343 | −8.288 | 5.261 | 54.972 | 1.00 | 21.41 |
| ATOM | 9059 | O | SER | 1343 | −8.924 | 6.099 | 55.620 | 1.00 | 20.45 |
| ATOM | 9060 | N | VAL | 1344 | −7.232 | 4.632 | 55.480 | 1.00 | 20.55 |
| ATOM | 9061 | CA | VAL | 1344 | −6.802 | 4.911 | 56.849 | 1.00 | 21.71 |
| ATOM | 9062 | CB | VAL | 1344 | −5.552 | 4.060 | 57.252 | 1.00 | 23.85 |
| ATOM | 9063 | CG1 | VAL | 1344 | −4.370 | 4.379 | 56.351 | 1.00 | 21.50 |
| ATOM | 9064 | CG2 | VAL | 1344 | −5.201 | 4.321 | 58.712 | 1.00 | 22.91 |
| ATOM | 9065 | C | VAL | 1344 | −6.501 | 6.398 | 57.057 | 1.00 | 22.37 |
| ATOM | 9066 | O | VAL | 1344 | −6.803 | 6.960 | 58.114 | 1.00 | 22.26 |
| ATOM | 9067 | N | ASN | 1345 | −5.929 | 7.038 | 56.041 | 1.00 | 22.04 |
| ATOM | 9068 | CA | ASN | 1345 | −5.596 | 8.458 | 56.123 | 1.00 | 22.35 |
| ATOM | 9069 | CB | ASN | 1345 | −4.665 | 8.845 | 54.974 | 1.00 | 22.83 |
| ATOM | 9070 | CG | ASN | 1345 | −3.349 | 8.103 | 55.030 | 1.00 | 22.33 |
| ATOM | 9071 | OD1 | ASN | 1345 | −2.566 | 8.288 | 55.958 | 1.00 | 23.07 |
| ATOM | 9072 | ND2 | ASN | 1345 | −3.103 | 7.248 | 54.045 | 1.00 | 22.68 |
| ATOM | 9073 | C | ASN | 1345 | −6.840 | 9.336 | 56.103 | 1.00 | 24.24 |
| ATOM | 9074 | O | ASN | 1345 | −6.816 | 10.465 | 56.588 | 1.00 | 23.31 |
| ATOM | 9075 | N | ILE | 1346 | −7.923 | 8.817 | 55.534 | 1.00 | 24.67 |
| ATOM | 9076 | CA | ILE | 1346 | −9.179 | 9.561 | 55.477 | 1.00 | 26.60 |
| ATOM | 9077 | CB | ILE | 1346 | −10.155 | 8.949 | 54.447 | 1.00 | 25.22 |
| ATOM | 9078 | CG2 | ILE | 1346 | −11.529 | 9.593 | 54.580 | 1.00 | 25.45 |
| ATOM | 9079 | CG1 | ILE | 1346 | −9.604 | 9.121 | 53.031 | 1.00 | 26.82 |
| ATOM | 9080 | CD1 | ILE | 1346 | −9.646 | 10.548 | 52.518 | 1.00 | 27.54 |
| ATOM | 9081 | C | ILE | 1346 | −9.847 | 9.534 | 56.850 | 1.00 | 25.86 |
| ATOM | 9082 | O | ILE | 1346 | −10.311 | 10.565 | 57.337 | 1.00 | 27.66 |
| ATOM | 9083 | N | PHE | 1347 | −9.896 | 8.352 | 57.463 | 1.00 | 26.34 |
| ATOM | 9084 | CA | PHE | 1347 | −10.513 | 8.180 | 58.780 | 1.00 | 27.79 |
| ATOM | 9085 | CB | PHE | 1347 | −10.855 | 6.707 | 59.040 | 1.00 | 30.55 |
| ATOM | 9086 | CG | PHE | 1347 | −11.662 | 6.055 | 57.951 | 1.00 | 33.41 |
| ATOM | 9087 | CD1 | PHE | 1347 | −12.751 | 6.707 | 57.382 | 1.00 | 33.70 |
| ATOM | 9088 | CD2 | PHE | 1347 | −11.349 | 4.766 | 57.518 | 1.00 | 34.68 |
| ATOM | 9089 | CE1 | PHE | 1347 | −13.516 | 6.088 | 56.398 | 1.00 | 34.26 |
| ATOM | 9090 | CE2 | PHE | 1347 | −12.108 | 4.137 | 56.533 | 1.00 | 34.13 |
| ATOM | 9091 | CZ | PHE | 1347 | −13.193 | 4.799 | 55.973 | 1.00 | 35.64 |
| ATOM | 9092 | C | PHE | 1347 | −9.609 | 8.655 | 59.912 | 1.00 | 28.04 |
| ATOM | 9093 | O | PHE | 1347 | −10.062 | 8.822 | 61.044 | 1.00 | 25.56 |
| ATOM | 9094 | N | GLY | 1348 | −8.329 | 8.852 | 59.609 | 1.00 | 28.73 |
| ATOM | 9095 | CA | GLY | 1348 | −7.387 | 9.290 | 60.625 | 1.00 | 30.19 |
| ATOM | 9096 | C | GLY | 1348 | −6.961 | 8.147 | 61.531 | 1.00 | 31.20 |
| ATOM | 9097 | O | GLY | 1348 | −6.574 | 8.361 | 62.679 | 1.00 | 32.36 |
| ATOM | 9098 | N | GLY | 1349 | −7.039 | 6.923 | 61.019 | 1.00 | 32.22 |
| ATOM | 9099 | CA | GLY | 1349 | −6.651 | 5.770 | 61.812 | 1.00 | 33.19 |
| ATOM | 9100 | C | GLY | 1349 | −7.580 | 4.592 | 61.597 | 1.00 | 34.56 |
| ATOM | 9101 | O | GLY | 1349 | −8.482 | 4.649 | 60.762 | 1.00 | 32.55 |
| ATOM | 9102 | N | TYR | 1350 | −7.359 | 3.517 | 62.346 | 1.00 | 35.88 |
| ATOM | 9103 | CA | TYR | 1350 | −8.195 | 2.333 | 62.226 | 1.00 | 36.67 |
| ATOM | 9104 | CB | TYR | 1350 | −7.336 | 1.069 | 62.293 | 1.00 | 36.59 |
| ATOM | 9105 | CG | TYR | 1350 | −6.220 | 1.046 | 61.271 | 1.00 | 38.85 |
| ATOM | 9106 | CD1 | TYR | 1350 | −4.989 | 1.643 | 61.542 | 1.00 | 38.83 |
| ATOM | 9107 | CE1 | TYR | 1350 | −3.969 | 1.661 | 60.592 | 1.00 | 38.99 |
| ATOM | 9108 | CD2 | TYR | 1350 | −6.406 | 0.463 | 60.017 | 1.00 | 38.83 |
| ATOM | 9109 | CE2 | TYR | 1350 | −5.389 | 0.478 | 59.054 | 1.00 | 38.92 |
| ATOM | 9110 | CZ | TYR | 1350 | −4.177 | 1.079 | 59.350 | 1.00 | 38.88 |
| ATOM | 9111 | OH | TYR | 1350 | −3.174 | 1.117 | 58.407 | 1.00 | 38.09 |
| ATOM | 9112 | C | TYR | 1350 | −9.239 | 2.327 | 63.336 | 1.00 | 37.58 |
| ATOM | 9113 | O | TYR | 1350 | −9.110 | 1.612 | 64.330 | 1.00 | 38.34 |
| ATOM | 9114 | N | LYS | 1351 | −10.278 | 3.132 | 63.150 | 1.00 | 37.80 |
| ATOM | 9115 | CA | LYS | 1351 | −11.349 | 3.254 | 64.128 | 1.00 | 37.85 |
| ATOM | 9116 | CB | LYS | 1351 | −11.650 | 4.738 | 64.354 | 1.00 | 39.96 |
| ATOM | 9117 | CG | LYS | 1351 | −10.399 | 5.592 | 64.529 | 1.00 | 41.93 |
| ATOM | 9118 | CD | LYS | 1351 | −10.671 | 7.063 | 64.253 | 1.00 | 43.31 |
| ATOM | 9119 | CE | LYS | 1351 | −9.373 | 7.860 | 64.209 | 1.00 | 44.78 |
| ATOM | 9120 | NZ | LYS | 1351 | −9.596 | 9.288 | 63.846 | 1.00 | 44.41 |
| ATOM | 9121 | C | LYS | 1351 | −12.612 | 2.531 | 63.648 | 1.00 | 37.02 |
| ATOM | 9122 | O | LYS | 1351 | −12.809 | 2.329 | 62.449 | 1.00 | 35.65 |
| ATOM | 9123 | N | VAL | 1352 | −13.461 | 2.145 | 64.595 | 1.00 | 36.10 |
| ATOM | 9124 | CA | VAL | 1352 | −14.705 | 1.456 | 64.280 | 1.00 | 35.05 |
| ATOM | 9125 | CB | VAL | 1352 | −15.483 | 1.100 | 65.569 | 1.00 | 35.70 |
| ATOM | 9126 | CG1 | VAL | 1352 | −16.731 | 0.303 | 65.232 | 1.00 | 34.09 |
| ATOM | 9127 | CG2 | VAL | 1352 | −14.586 | 0.314 | 66.512 | 1.00 | 35.71 |
| ATOM | 9128 | C | VAL | 1352 | −15.570 | 2.358 | 63.405 | 1.00 | 34.29 |
| ATOM | 9129 | O | VAL | 1352 | −15.604 | 3.576 | 63.595 | 1.00 | 32.86 |
| ATOM | 9130 | N | GLN | 1353 | −16.259 | 1.755 | 62.441 | 1.00 | 33.15 |
| ATOM | 9131 | CA | GLN | 1353 | −17.121 | 2.493 | 61.523 | 1.00 | 33.61 |
| ATOM | 9132 | CB | GLN | 1353 | −16.664 | 2.248 | 60.082 | 1.00 | 35.20 |
| ATOM | 9133 | CG | GLN | 1353 | −16.394 | 3.503 | 59.269 | 1.00 | 37.01 |
| ATOM | 9134 | CD | GLN | 1353 | −15.374 | 4.407 | 59.920 | 1.00 | 37.73 |
| ATOM | 9135 | OE1 | GLN | 1353 | −14.339 | 3.948 | 60.405 | 1.00 | 39.42 |
| ATOM | 9136 | NE2 | GLN | 1353 | −15.656 | 5.703 | 59.929 | 1.00 | 39.06 |
| ATOM | 9137 | C | GLN | 1353 | −18.568 | 2.038 | 61.678 | 1.00 | 32.00 |
| ATOM | 9138 | O | GLN | 1353 | −18.839 | 1.006 | 62.289 | 1.00 | 32.23 |
| ATOM | 9139 | N | GLY | 1354 | −19.497 | 2.810 | 61.124 | 1.00 | 32.12 |
| ATOM | 9140 | CA | GLY | 1354 | −20.901 | 2.445 | 61.207 | 1.00 | 32.69 |
| ATOM | 9141 | C | GLY | 1354 | −21.624 | 2.931 | 62.453 | 1.00 | 33.83 |
| ATOM | 9142 | O | GLY | 1354 | −22.812 | 2.658 | 62.634 | 1.00 | 33.12 |
| ATOM | 9143 | N | ARG | 1355 | −20.912 | 3.646 | 63.317 | 1.00 | 33.73 |
| ATOM | 9144 | CA | ARG | 1355 | −21.509 | 4.168 | 64.538 | 1.00 | 35.35 |
| ATOM | 9145 | CB | ARG | 1355 | −20.420 | 4.610 | 65.523 | 1.00 | 36.22 |
| ATOM | 9146 | CG | ARG | 1355 | −19.601 | 3.469 | 66.116 | 1.00 | 37.99 |
| ATOM | 9147 | CD | ARG | 1355 | −19.623 | 3.530 | 67.636 | 1.00 | 37.53 |
| ATOM | 9148 | NE | ARG | 1355 | −18.923 | 2.412 | 68.266 | 1.00 | 38.27 |
| ATOM | 9149 | CZ | ARG | 1355 | −17.601 | 2.278 | 68.305 | 1.00 | 37.61 |
| ATOM | 9150 | NH1 | ARG | 1355 | −16.819 | 3.195 | 67.752 | 1.00 | 38.90 |
| ATOM | 9151 | NH2 | ARG | 1355 | −17.062 | 1.228 | 68.905 | 1.00 | 38.50 |
| ATOM | 9152 | C | ARG | 1355 | −22.417 | 5.353 | 64.221 | 1.00 | 34.99 |
| ATOM | 9153 | O | ARG | 1355 | −21.988 | 6.326 | 63.601 | 1.00 | 33.87 |
| ATOM | 9154 | N | GLY | 1356 | −23.673 | 5.268 | 64.645 | 1.00 | 35.30 |
| ATOM | 9155 | CA | GLY | 1356 | −24.603 | 6.353 | 64.388 | 1.00 | 36.39 |
| ATOM | 9156 | C | GLY | 1356 | −25.609 | 5.991 | 63.316 | 1.00 | 36.56 |
| ATOM | 9157 | O | GLY | 1356 | −25.403 | 5.044 | 62.556 | 1.00 | 37.08 |
| ATOM | 9158 | N | ASP | 1357 | −26.704 | 6.741 | 63.251 | 1.00 | 35.94 |
| ATOM | 9159 | CA | ASP | 1357 | −27.730 | 6.470 | 62.257 | 1.00 | 35.55 |
| ATOM | 9160 | CB | ASP | 1357 | −29.029 | 7.206 | 62.596 | 1.00 | 38.36 |
| ATOM | 9161 | CG | ASP | 1357 | −29.677 | 6.687 | 63.863 | 1.00 | 39.91 |
| ATOM | 9162 | OD1 | ASP | 1357 | −29.647 | 5.458 | 64.092 | 1.00 | 41.41 |
| ATOM | 9163 | OD2 | ASP | 1357 | −30.229 | 7.504 | 64.624 | 1.00 | 42.41 |
| ATOM | 9164 | C | ASP | 1357 | −27.280 | 6.860 | 60.859 | 1.00 | 33.61 |
| ATOM | 9165 | O | ASP | 1357 | −27.444 | 6.092 | 59.915 | 1.00 | 34.09 |
| ATOM | 9166 | N | GLU | 1358 | −26.710 | 8.051 | 60.723 | 1.00 | 31.82 |
| ATOM | 9167 | CA | GLU | 1358 | −26.257 | 8.517 | 59.419 | 1.00 | 32.56 |
| ATOM | 9168 | CB | GLU | 1358 | −25.702 | 9.938 | 59.531 | 1.00 | 35.82 |
| ATOM | 9169 | CG | GLU | 1358 | −25.286 | 10.550 | 58.206 | 1.00 | 41.80 |
| ATOM | 9170 | CD | GLU | 1358 | −24.844 | 11.992 | 58.347 | 1.00 | 44.28 |
| ATOM | 9171 | OE1 | GLU | 1358 | −25.675 | 12.835 | 58.748 | 1.00 | 47.82 |
| ATOM | 9172 | OE2 | GLU | 1358 | −23.665 | 12.286 | 58.062 | 1.00 | 47.55 |
| ATOM | 9173 | C | GLU | 1358 | −25.203 | 7.584 | 58.819 | 1.00 | 31.25 |
| ATOM | 9174 | O | GLU | 1358 | −25.300 | 7.199 | 57.652 | 1.00 | 29.54 |
| ATOM | 9175 | N | ALA | 1359 | −24.205 | 7.218 | 59.619 | 1.00 | 28.61 |
| ATOM | 9176 | CA | ALA | 1359 | −23.141 | 6.327 | 59.162 | 1.00 | 26.73 |
| ATOM | 9177 | CB | ALA | 1359 | −22.033 | 6.251 | 60.213 | 1.00 | 27.02 |
| ATOM | 9178 | C | ALA | 1359 | −23.694 | 4.931 | 58.887 | 1.00 | 25.02 |
| ATOM | 9179 | O | ALA | 1359 | −23.314 | 4.281 | 57.910 | 1.00 | 25.54 |
| ATOM | 9180 | N | GLY | 1360 | −24.591 | 4.483 | 59.759 | 1.00 | 22.90 |
| ATOM | 9181 | CA | GLY | 1360 | −25.191 | 3.171 | 59.606 | 1.00 | 22.42 |
| ATOM | 9182 | C | GLY | 1360 | −26.036 | 3.070 | 58.350 | 1.00 | 23.63 |
| ATOM | 9183 | O | GLY | 1360 | −25.874 | 2.143 | 57.552 | 1.00 | 21.50 |
| ATOM | 9184 | N | ASP | 1361 | −26.940 | 4.029 | 58.175 | 1.00 | 23.37 |
| ATOM | 9185 | CA | ASP | 1361 | −27.809 | 4.055 | 57.010 | 1.00 | 24.97 |
| ATOM | 9186 | CB | ASP | 1361 | −28.776 | 5.242 | 57.082 | 1.00 | 25.15 |
| ATOM | 9187 | CG | ASP | 1361 | −29.778 | 5.109 | 58.212 | 1.00 | 25.98 |
| ATOM | 9188 | OD1 | ASP | 1361 | −30.037 | 3.963 | 58.634 | 1.00 | 24.04 |
| ATOM | 9189 | OD2 | ASP | 1361 | −30.312 | 6.148 | 58.662 | 1.00 | 26.75 |
| ATOM | 9190 | C | ASP | 1361 | −27.001 | 4.128 | 55.715 | 1.00 | 24.67 |
| ATOM | 9191 | O | ASP | 1361 | −27.418 | 3.599 | 54.682 | 1.00 | 23.97 |
| ATOM | 9192 | N | GLN | 1362 | −25.843 | 4.775 | 55.763 | 1.00 | 23.06 |
| ATOM | 9193 | CA | GLN | 1362 | −25.021 | 4.882 | 54.563 | 1.00 | 23.44 |
| ATOM | 9194 | CB | GLN | 1362 | −23.890 | 5.896 | 54.766 | 1.00 | 23.04 |
| ATOM | 9195 | CG | GLN | 1362 | −23.177 | 6.276 | 53.474 | 1.00 | 25.55 |
| ATOM | 9196 | CD | GLN | 1362 | −24.113 | 6.925 | 52.464 | 1.00 | 29.06 |
| ATOM | 9197 | OE1 | GLN | 1362 | −24.758 | 7.933 | 52.760 | 1.00 | 28.92 |
| ATOM | 9198 | NE2 | GLN | 1362 | −24.195 | 6.345 | 51.266 | 1.00 | 29.63 |
| ATOM | 9199 | C | GLN | 1362 | −24.439 | 3.519 | 54.194 | 1.00 | 23.54 |
| ATOM | 9200 | O | GLN | 1362 | −24.409 | 3.157 | 53.019 | 1.00 | 23.64 |
| ATOM | 9201 | N | LEU | 1363 | −23.984 | 2.764 | 55.190 | 1.00 | 24.10 |
| ATOM | 9202 | CA | LEU | 1363 | −23.418 | 1.441 | 54.932 | 1.00 | 25.78 |
| ATOM | 9203 | CB | LEU | 1363 | −22.840 | 0.831 | 56.214 | 1.00 | 27.37 |
| ATOM | 9204 | CG | LEU | 1363 | −21.488 | 1.337 | 56.715 | 1.00 | 30.02 |
| ATOM | 9205 | CD1 | LEU | 1363 | −21.078 | 0.539 | 57.948 | 1.00 | 29.46 |
| ATOM | 9206 | CD2 | LEU | 1363 | −20.436 | 1.184 | 55.628 | 1.00 | 30.43 |
| ATOM | 9207 | C | LEU | 1363 | −24.453 | 0.485 | 54.354 | 1.00 | 25.61 |
| ATOM | 9208 | O | LEU | 1363 | −24.186 | −0.215 | 53.378 | 1.00 | 24.86 |
| ATOM | 9209 | N | LEU | 1364 | −25.633 | 0.446 | 54.965 | 1.00 | 25.26 |
| ATOM | 9210 | CA | LEU | 1364 | −26.690 | −0.433 | 54.486 | 1.00 | 25.78 |
| ATOM | 9211 | CB | LEU | 1364 | −27.940 | −0.270 | 55.355 | 1.00 | 28.25 |
| ATOM | 9212 | CG | LEU | 1364 | −28.877 | −1.483 | 55.466 | 1.00 | 30.43 |
| ATOM | 9213 | CD1 | LEU | 1364 | −29.981 | −1.183 | 56.470 | 1.00 | 33.01 |
| ATOM | 9214 | CD2 | LEU | 1364 | −29.468 | −1.809 | 54.117 | 1.00 | 31.42 |
| ATOM | 9215 | C | LEU | 1364 | −26.996 | −0.081 | 53.030 | 1.00 | 24.42 |
| ATOM | 9216 | O | LEU | 1364 | −27.124 | −0.963 | 52.180 | 1.00 | 24.75 |
| ATOM | 9217 | N | SER | 1365 | −27.091 | 1.216 | 52.748 | 1.00 | 24.25 |
| ATOM | 9218 | CA | SER | 1365 | −27.372 | 1.696 | 51.399 | 1.00 | 22.74 |
| ATOM | 9219 | CB | SER | 1365 | −27.440 | 3.226 | 51.388 | 1.00 | 25.49 |
| ATOM | 9220 | OG | SER | 1365 | −27.711 | 3.713 | 50.085 | 1.00 | 30.28 |
| ATOM | 9221 | C | SER | 1365 | −26.288 | 1.225 | 50.436 | 1.00 | 20.66 |
| ATOM | 9222 | O | SER | 1365 | −26.581 | 0.731 | 49.343 | 1.00 | 17.79 |
| ATOM | 9223 | N | ASP | 1366 | −25.033 | 1.397 | 50.841 | 1.00 | 19.04 |
| ATOM | 9224 | CA | ASP | 1366 | −23.902 | 0.971 | 50.013 | 1.00 | 18.22 |
| ATOM | 9225 | CB | ASP | 1366 | −22.564 | 1.389 | 50.645 | 1.00 | 19.61 |
| ATOM | 9226 | CG | ASP | 1366 | −22.294 | 2.890 | 50.553 | 1.00 | 20.96 |
| ATOM | 9227 | OD1 | ASP | 1366 | −22.831 | 3.569 | 49.652 | 1.00 | 20.14 |
| ATOM | 9228 | OD2 | ASP | 1366 | −21.508 | 3.393 | 51.384 | 1.00 | 19.72 |
| ATOM | 9229 | C | ASP | 1366 | −23.914 | −0.547 | 49.824 | 1.00 | 16.69 |
| ATOM | 9230 | O | ASP | 1366 | −23.643 | −1.040 | 48.728 | 1.00 | 18.02 |
| ATOM | 9231 | N | ALA | 1367 | −24.216 | −1.291 | 50.885 | 1.00 | 15.72 |
| ATOM | 9232 | CA | ALA | 1367 | −24.244 | −2.748 | 50.783 | 1.00 | 16.03 |
| ATOM | 9233 | CB | ALA | 1367 | −24.598 | −3.372 | 52.136 | 1.00 | 16.32 |
| ATOM | 9234 | C | ALA | 1367 | −25.245 | −3.181 | 49.717 | 1.00 | 17.08 |
| ATOM | 9235 | O | ALA | 1367 | −24.944 | −4.032 | 48.878 | 1.00 | 15.80 |
| ATOM | 9236 | N | LEU | 1368 | −26.436 | −2.588 | 49.745 | 1.00 | 16.63 |
| ATOM | 9237 | CA | LEU | 1368 | −27.473 | −2.902 | 48.759 | 1.00 | 15.83 |
| ATOM | 9238 | CB | LEU | 1368 | −28.775 | −2.201 | 49.131 | 1.00 | 17.90 |
| ATOM | 9239 | CG | LEU | 1368 | −29.457 | −2.782 | 50.363 | 1.00 | 18.04 |
| ATOM | 9240 | CD1 | LEU | 1368 | −30.451 | −1.771 | 50.910 | 1.00 | 22.56 |
| ATOM | 9241 | CD2 | LEU | 1368 | −30.137 | −4.097 | 49.992 | 1.00 | 18.80 |
| ATOM | 9242 | C | LEU | 1368 | −27.060 | −2.475 | 47.360 | 1.00 | 15.44 |
| ATOM | 9243 | O | LEU | 1368 | −27.309 | −3.189 | 46.390 | 1.00 | 17.08 |
| ATOM | 9244 | N | ALA | 1369 | −26.437 | −1.304 | 47.265 | 1.00 | 15.14 |
| ATOM | 9245 | CA | ALA | 1369 | −25.985 | −0.781 | 45.984 | 1.00 | 15.71 |
| ATOM | 9246 | CB | ALA | 1369 | −25.393 | 0.609 | 46.164 | 1.00 | 16.16 |
| ATOM | 9247 | C | ALA | 1369 | −24.948 | −1.714 | 45.372 | 1.00 | 15.06 |
| ATOM | 9248 | O | ALA | 1369 | −24.979 | −1.985 | 44.172 | 1.00 | 13.93 |
| ATOM | 9249 | N | LEU | 1370 | −24.025 | −2.201 | 46.196 | 1.00 | 15.56 |
| ATOM | 9250 | CA | LEU | 1370 | −22.990 | −3.106 | 45.702 | 1.00 | 15.57 |
| ATOM | 9251 | CB | LEU | 1370 | −21.962 | −3.389 | 46.805 | 1.00 | 17.04 |
| ATOM | 9252 | CG | LEU | 1370 | −21.115 | −2.176 | 47.231 | 1.00 | 17.64 |
| ATOM | 9253 | CD1 | LEU | 1370 | −20.257 | −2.520 | 48.450 | 1.00 | 14.36 |
| ATOM | 9254 | CD2 | LEU | 1370 | −20.236 | −1.745 | 46.077 | 1.00 | 17.55 |
| ATOM | 9255 | C | LEU | 1370 | −23.639 | −4.395 | 45.228 | 1.00 | 16.16 |
| ATOM | 9256 | O | LEU | 1370 | −23.306 | −4.917 | 44.160 | 1.00 | 14.48 |
| ATOM | 9257 | N | GLU | 1371 | −24.573 | −4.912 | 46.020 | 1.00 | 15.93 |
| ATOM | 9258 | CA | GLU | 1371 | −25.269 | −6.142 | 45.647 | 1.00 | 16.46 |
| ATOM | 9259 | CB | GLU | 1371 | −26.264 | −6.540 | 46.745 | 1.00 | 17.16 |
| ATOM | 9260 | CG | GLU | 1371 | −27.134 | −7.735 | 46.381 | 1.00 | 20.30 |
| ATOM | 9261 | CD | GLU | 1371 | −28.084 | −8.098 | 47.491 | 1.00 | 19.27 |
| ATOM | 9262 | OE1 | GLU | 1371 | −28.878 | −7.224 | 47.897 | 1.00 | 18.29 |
| ATOM | 9263 | OE2 | GLU | 1371 | −28.029 | −9.248 | 47.964 | 1.00 | 23.87 |
| ATOM | 9264 | C | GLU | 1371 | −26.007 | −5.960 | 44.319 | 1.00 | 16.54 |
| ATOM | 9265 | O | GLU | 1371 | −25.894 | −6.789 | 43.409 | 1.00 | 16.17 |
| ATOM | 9266 | N | ALA | 1372 | −26.757 | −4.869 | 44.205 | 1.00 | 16.06 |
| ATOM | 9267 | CA | ALA | 1372 | −27.516 | −4.601 | 42.987 | 1.00 | 16.73 |
| ATOM | 9268 | CB | ALA | 1372 | −28.354 | −3.328 | 43.161 | 1.00 | 16.45 |
| ATOM | 9269 | C | ALA | 1372 | −26.585 | −4.460 | 41.790 | 1.00 | 16.56 |
| ATOM | 9270 | O | ALA | 1372 | −26.953 | −4.791 | 40.660 | 1.00 | 15.51 |
| ATOM | 9271 | N | ALA | 1373 | −25.377 | −3.961 | 42.044 | 1.00 | 14.61 |
| ATOM | 9272 | CA | ALA | 1373 | −24.382 | −3.758 | 40.987 | 1.00 | 14.87 |
| ATOM | 9273 | CB | ALA | 1373 | −23.253 | −2.858 | 41.507 | 1.00 | 15.59 |
| ATOM | 9274 | C | ALA | 1373 | −23.804 | −5.077 | 40.477 | 1.00 | 14.66 |
| ATOM | 9275 | O | ALA | 1373 | −23.232 | −5.133 | 39.386 | 1.00 | 15.82 |
| ATOM | 9276 | N | GLY | 1374 | −23.935 | −6.132 | 41.273 | 1.00 | 14.12 |
| ATOM | 9277 | CA | GLY | 1374 | −23.427 | −7.423 | 40.846 | 1.00 | 14.74 |
| ATOM | 9278 | C | GLY | 1374 | −22.460 | −8.120 | 41.789 | 1.00 | 15.58 |
| ATOM | 9279 | O | GLY | 1374 | −22.046 | −9.250 | 41.513 | 1.00 | 17.25 |
| ATOM | 9280 | N | ALA | 1375 | −22.081 | −7.468 | 42.888 | 1.00 | 15.59 |
| ATOM | 9281 | CA | ALA | 1375 | −21.167 | −8.098 | 43.847 | 1.00 | 15.31 |
| ATOM | 9282 | CB | ALA | 1375 | −20.901 | −7.167 | 45.031 | 1.00 | 12.55 |
| ATOM | 9283 | C | ALA | 1375 | −21.815 | −9.390 | 44.336 | 1.00 | 15.80 |
| ATOM | 9284 | O | ALA | 1375 | −23.000 | −9.408 | 44.659 | 1.00 | 17.75 |
| ATOM | 9285 | N | GLN | 1376 | −21.041 | −10.469 | 44.393 | 1.00 | 16.08 |
| ATOM | 9286 | CA | GLN | 1376 | −21.584 | −11.756 | 44.825 | 1.00 | 16.60 |
| ATOM | 9287 | CB | GLN | 1376 | −21.090 | −12.857 | 43.887 | 1.00 | 16.95 |
| ATOM | 9288 | CG | GLN | 1376 | −21.526 | −12.625 | 42.441 | 1.00 | 22.74 |
| ATOM | 9289 | CD | GLN | 1376 | −20.999 | −13.668 | 41.484 | 1.00 | 24.07 |
| ATOM | 9290 | OE1 | GLN | 1376 | −21.457 | −14.809 | 41.477 | 1.00 | 29.42 |
| ATOM | 9291 | NE2 | GLN | 1376 | −20.030 | −13.281 | 40.665 | 1.00 | 27.21 |
| ATOM | 9292 | C | GLN | 1376 | −21.217 | −12.076 | 46.269 | 1.00 | 17.00 |
| ATOM | 9293 | O | GLN | 1376 | −21.664 | −13.079 | 46.829 | 1.00 | 17.05 |
| ATOM | 9294 | N | LEU | 1377 | −20.411 | −11.199 | 46.860 | 1.00 | 14.88 |
| ATOM | 9295 | CA | LEU | 1377 | −19.958 | −11.337 | 48.232 | 1.00 | 16.52 |
| ATOM | 9296 | CB | LEU | 1377 | −18.675 | −12.164 | 48.280 | 1.00 | 21.20 |
| ATOM | 9297 | CG | LEU | 1377 | −18.797 | −13.672 | 48.435 | 1.00 | 22.45 |
| ATOM | 9298 | CD1 | LEU | 1377 | −17.441 | −14.317 | 48.174 | 1.00 | 22.86 |
| ATOM | 9299 | CD2 | LEU | 1377 | −19.279 | −13.991 | 49.840 | 1.00 | 22.77 |
| ATOM | 9300 | C | LEU | 1377 | −19.674 | −9.964 | 48.807 | 1.00 | 15.17 |
| ATOM | 9301 | O | LEU | 1377 | −19.404 | −9.017 | 48.068 | 1.00 | 15.26 |
| ATOM | 9302 | N | LEU | 1378 | −19.734 | −9.856 | 50.129 | 1.00 | 15.03 |
| ATOM | 9303 | CA | LEU | 1378 | −19.445 | −8.588 | 50.792 | 1.00 | 15.26 |
| ATOM | 9304 | CB | LEU | 1378 | −20.750 | −7.840 | 51.099 | 1.00 | 15.07 |
| ATOM | 9305 | CG | LEU | 1378 | −20.584 | −6.620 | 52.011 | 1.00 | 15.89 |
| ATOM | 9306 | CD1 | LEU | 1378 | −19.891 | −5.491 | 51.246 | 1.00 | 17.24 |
| ATOM | 9307 | CD2 | LEU | 1378 | −21.961 | −6.154 | 52.507 | 1.00 | 19.57 |
| ATOM | 9308 | C | LEU | 1378 | −18.679 | −8.808 | 52.090 | 1.00 | 15.81 |
| ATOM | 9309 | O | LEU | 1378 | −19.048 | −9.656 | 52.900 | 1.00 | 16.07 |
| ATOM | 9310 | N | VAL | 1379 | −17.599 | −8.060 | 52.282 | 1.00 | 16.13 |
| ATOM | 9311 | CA | VAL | 1379 | −16.845 | −8.160 | 53.523 | 1.00 | 16.26 |
| ATOM | 9312 | CB | VAL | 1379 | −15.297 | −8.136 | 53.275 | 1.00 | 14.82 |
| ATOM | 9313 | CG1 | VAL | 1379 | −14.567 | −7.886 | 54.588 | 1.00 | 14.92 |
| ATOM | 9314 | CG2 | VAL | 1379 | −14.830 | −9.493 | 52.691 | 1.00 | 13.84 |
| ATOM | 9315 | C | VAL | 1379 | −17.245 | −6.955 | 54.383 | 1.00 | 16.23 |
| ATOM | 9316 | O | VAL | 1379 | −17.366 | −5.838 | 53.876 | 1.00 | 14.40 |
| ATOM | 9317 | N | LEU | 1380 | −17.502 | −7.200 | 55.667 | 1.00 | 17.52 |
| ATOM | 9318 | CA | LEU | 1380 | −17.869 | −6.150 | 56.626 | 1.00 | 18.33 |
| ATOM | 9319 | CB | LEU | 1380 | −19.228 | −6.426 | 57.264 | 1.00 | 23.25 |
| ATOM | 9320 | CG | LEU | 1380 | −20.451 | −5.763 | 56.649 | 1.00 | 27.26 |
| ATOM | 9321 | CD1 | LEU | 1380 | −21.679 | −6.205 | 57.436 | 1.00 | 29.17 |
| ATOM | 9322 | CD2 | LEU | 1380 | −20.310 | −4.237 | 56.689 | 1.00 | 28.64 |
| ATOM | 9323 | C | LEU | 1380 | −16.820 | −6.151 | 57.729 | 1.00 | 17.93 |
| ATOM | 9324 | O | LEU | 1380 | −16.671 | −7.142 | 58.438 | 1.00 | 17.27 |
| ATOM | 9325 | N | GLU | 1381 | −16.126 | −5.032 | 57.901 | 1.00 | 18.24 |
| ATOM | 9326 | CA | GLU | 1381 | −15.058 | −4.951 | 58.890 | 1.00 | 18.15 |
| ATOM | 9327 | CB | GLU | 1381 | −13.735 | −4.706 | 58.154 | 1.00 | 19.99 |
| ATOM | 9328 | CG | GLU | 1381 | −12.535 | −4.399 | 59.042 | 1.00 | 19.58 |
| ATOM | 9329 | CD | GLU | 1381 | −11.214 | −4.485 | 58.288 | 1.00 | 21.41 |
| ATOM | 9330 | OE1 | GLU | 1381 | −11.236 | −4.456 | 57.042 | 1.00 | 20.29 |
| ATOM | 9331 | OE2 | GLU | 1381 | −10.152 | −4.579 | 58.939 | 1.00 | 22.00 |
| ATOM | 9332 | C | GLU | 1381 | −15.222 | −3.910 | 59.995 | 1.00 | 18.95 |
| ATOM | 9333 | O | GLU | 1381 | −15.451 | −2.733 | 59.725 | 1.00 | 17.56 |
| ATOM | 9334 | N | CYS | 1382 | −15.094 | −4.355 | 61.242 | 1.00 | 18.95 |
| ATOM | 9335 | CA | CYS | 1382 | −15.168 | −3.456 | 62.388 | 1.00 | 20.18 |
| ATOM | 9336 | CB | CYS | 1382 | −13.858 | −2.676 | 62.501 | 1.00 | 20.94 |
| ATOM | 9337 | SG | CYS | 1382 | −12.444 | −3.736 | 62.885 | 1.00 | 25.87 |
| ATOM | 9338 | C | CYS | 1382 | −16.344 | −2.488 | 62.383 | 1.00 | 19.45 |
| ATOM | 9339 | O | CYS | 1382 | −16.181 | −1.279 | 62.191 | 1.00 | 19.01 |
| ATOM | 9340 | N | VAL | 1383 | −17.523 | −3.038 | 62.631 | 1.00 | 20.89 |
| ATOM | 9341 | CA | VAL | 1383 | −18.753 | −2.272 | 62.665 | 1.00 | 21.85 |
| ATOM | 9342 | CB | VAL | 1383 | −19.513 | −2.440 | 61.324 | 1.00 | 23.50 |
| ATOM | 9343 | CG1 | VAL | 1383 | −20.222 | −3.785 | 61.286 | 1.00 | 18.79 |
| ATOM | 9344 | CG2 | VAL | 1383 | −20.471 | −1.295 | 61.113 | 1.00 | 26.50 |
| ATOM | 9345 | C | VAL | 1383 | −19.576 | −2.863 | 63.809 | 1.00 | 22.30 |
| ATOM | 9346 | O | VAL | 1383 | −19.434 | −4.042 | 64.131 | 1.00 | 22.63 |
| ATOM | 9347 | N | PRO | 1384 | −20.432 | −2.050 | 64.454 | 1.00 | 22.70 |
| ATOM | 9348 | CD | PRO | 1384 | −20.747 | −0.631 | 64.237 | 1.00 | 21.90 |
| ATOM | 9349 | CA | PRO | 1384 | −21.231 | −2.608 | 65.548 | 1.00 | 23.71 |
| ATOM | 9350 | CB | PRO | 1384 | −22.140 | −1.441 | 65.952 | 1.00 | 24.27 |
| ATOM | 9351 | CG | PRO | 1384 | −22.162 | −0.556 | 64.735 | 1.00 | 26.55 |
| ATOM | 9352 | C | PRO | 1384 | −22.009 | −3.841 | 65.101 | 1.00 | 24.45 |
| ATOM | 9353 | O | PRO | 1384 | −22.470 | −3.914 | 63.961 | 1.00 | 23.74 |
| ATOM | 9354 | N | VAL | 1385 | −22.140 | −4.808 | 66.003 | 1.00 | 24.71 |
| ATOM | 9355 | CA | VAL | 1385 | −22.856 | −6.050 | 65.727 | 1.00 | 26.23 |
| ATOM | 9356 | CB | VAL | 1385 | −22.984 | −6.917 | 66.991 | 1.00 | 27.89 |
| ATOM | 9357 | CG1 | VAL | 1385 | −23.606 | −8.261 | 66.632 | 1.00 | 27.76 |
| ATOM | 9358 | CG2 | VAL | 1385 | −21.626 | −7.101 | 67.635 | 1.00 | 29.95 |
| ATOM | 9359 | C | VAL | 1385 | −24.263 | −5.783 | 65.219 | 1.00 | 27.91 |
| ATOM | 9360 | O | VAL | 1385 | −24.772 | −6.513 | 64.366 | 1.00 | 27.26 |
| ATOM | 9361 | N | GLU | 1386 | −24.884 | −4.734 | 65.754 | 1.00 | 27.87 |
| ATOM | 9362 | CA | GLU | 1386 | −26.241 | −4.372 | 65.367 | 1.00 | 30.32 |
| ATOM | 9363 | CB | GLU | 1386 | −26.717 | −3.129 | 66.133 | 1.00 | 31.48 |
| ATOM | 9364 | CG | GLU | 1386 | −26.182 | −2.988 | 67.553 | 1.00 | 37.12 |
| ATOM | 9365 | CD | GLU | 1386 | −26.077 | −4.309 | 68.280 | 1.00 | 39.44 |
| ATOM | 9366 | OE1 | GLU | 1386 | −27.084 | −5.051 | 68.325 | 1.00 | 42.32 |
| ATOM | 9367 | OE2 | GLU | 1386 | −24.984 | −4.599 | 68.810 | 1.00 | 40.64 |
| ATOM | 9368 | C | GLU | 1386 | −26.291 | −4.088 | 63.872 | 1.00 | 29.04 |
| ATOM | 9369 | O | GLU | 1386 | −27.157 | −4.601 | 63.168 | 1.00 | 29.05 |
| ATOM | 9370 | N | LEU | 1387 | −25.353 | −3.271 | 63.396 | 1.00 | 27.86 |
| ATOM | 9371 | CA | LEU | 1387 | −25.290 | −2.912 | 61.982 | 1.00 | 26.77 |
| ATOM | 9372 | CB | LEU | 1387 | −24.193 | −1.870 | 61.740 | 1.00 | 29.38 |
| ATOM | 9373 | CG | LEU | 1387 | −24.497 | −0.767 | 60.722 | 1.00 | 31.48 |
| ATOM | 9374 | CD1 | LEU | 1387 | −23.294 | 0.148 | 60.577 | 1.00 | 29.59 |
| ATOM | 9375 | CD2 | LEU | 1387 | −24.876 | −1.369 | 59.391 | 1.00 | 33.03 |
| ATOM | 9376 | C | LEU | 1387 | −25.019 | −4.148 | 61.128 | 1.00 | 26.22 |
| ATOM | 9377 | O | LEU | 1387 | −25.637 | −4.338 | 60.079 | 1.00 | 24.83 |
| ATOM | 9378 | N | ALA | 1388 | −24.082 | −4.982 | 61.572 | 1.00 | 24.74 |
| ATOM | 9379 | CA | ALA | 1388 | −23.758 | −6.199 | 60.846 | 1.00 | 23.61 |
| ATOM | 9380 | CB | ALA | 1388 | −22.736 | −7.022 | 61.625 | 1.00 | 21.94 |
| ATOM | 9381 | C | ALA | 1388 | −25.041 | −7.002 | 60.648 | 1.00 | 23.83 |
| ATOM | 9382 | O | ALA | 1388 | −25.301 | −7.517 | 59.560 | 1.00 | 20.30 |
| ATOM | 9383 | N | LYS | 1389 | −25.844 | −7.096 | 61.705 | 1.00 | 25.43 |
| ATOM | 9384 | CA | LYS | 1389 | −27.114 | −7.824 | 61.656 | 1.00 | 27.87 |
| ATOM | 9385 | CB | LYS | 1389 | −27.853 | −7.694 | 62.992 | 1.00 | 32.12 |
| ATOM | 9386 | CG | LYS | 1389 | −27.057 | −8.133 | 64.203 | 1.00 | 38.20 |
| ATOM | 9387 | CD | LYS | 1389 | −27.773 | −7.773 | 65.500 | 1.00 | 41.46 |
| ATOM | 9388 | CE | LYS | 1389 | −26.986 | −8.255 | 66.710 | 1.00 | 43.04 |
| ATOM | 9389 | NZ | LYS | 1389 | −27.588 | −7.817 | 67.999 | 1.00 | 44.99 |
| ATOM | 9390 | C | LYS | 1389 | −27.999 | −7.252 | 60.556 | 1.00 | 26.60 |
| ATOM | 9391 | O | LYS | 1389 | −28.522 | −7.984 | 59.716 | 1.00 | 26.86 |
| ATOM | 9392 | N | ARG | 1390 | −28.160 | −5.932 | 60.580 | 1.00 | 26.01 |
| ATOM | 9393 | CA | ARG | 1390 | −28.980 | −5.216 | 59.608 | 1.00 | 25.62 |
| ATOM | 9394 | CB | ARG | 1390 | −28.846 | −3.704 | 59.830 | 1.00 | 28.15 |
| ATOM | 9395 | CG | ARG | 1390 | −28.910 | −3.255 | 61.284 | 1.00 | 33.06 |
| ATOM | 9396 | CD | ARG | 1390 | −30.061 | −2.299 | 61.530 | 1.00 | 31.58 |
| ATOM | 9397 | NE | ARG | 1390 | −30.047 | −1.150 | 60.627 | 1.00 | 34.15 |
| ATOM | 9398 | CZ | ARG | 1390 | −29.289 | −0.068 | 60.782 | 1.00 | 33.40 |
| ATOM | 9399 | NH1 | ARG | 1390 | −28.466 | 0.031 | 61.818 | 1.00 | 35.42 |
| ATOM | 9400 | NH2 | ARG | 1390 | −29.361 | 0.924 | 59.901 | 1.00 | 33.81 |
| ATOM | 9401 | C | ARG | 1390 | −28.566 | −5.541 | 58.175 | 1.00 | 24.93 |
| ATOM | 9402 | O | ARG | 1390 | −29.394 | −5.896 | 57.332 | 1.00 | 21.80 |
| ATOM | 9403 | N | ILE | 1391 | −27.271 | −5.407 | 57.904 | 1.00 | 24.32 |
| ATOM | 9404 | CA | ILE | 1391 | −26.736 | −5.663 | 56.570 | 1.00 | 23.53 |
| ATOM | 9405 | CB | ILE | 1391 | −25.248 | −5.277 | 56.501 | 1.00 | 24.79 |
| ATOM | 9406 | CG2 | ILE | 1391 | −24.677 | −5.627 | 55.130 | 1.00 | 22.88 |
| ATOM | 9407 | CG1 | ILE | 1391 | −25.098 | −3.779 | 56.789 | 1.00 | 24.38 |
| ATOM | 9408 | CD1 | ILE | 1391 | −23.661 | −3.314 | 56.863 | 1.00 | 30.37 |
| ATOM | 9409 | C | ILE | 1391 | −26.900 | −7.114 | 56.125 | 1.00 | 23.92 |
| ATOM | 9410 | O | ILE | 1391 | −27.323 | −7.383 | 55.001 | 1.00 | 22.87 |
| ATOM | 9411 | N | THR | 1392 | −26.570 | −8.053 | 57.003 | 1.00 | 23.07 |
| ATOM | 9412 | CA | THR | 1392 | −26.691 | −9.463 | 56.654 | 1.00 | 24.09 |
| ATOM | 9413 | CB | THR | 1392 | −26.165 | −10.362 | 57.801 | 1.00 | 25.31 |
| ATOM | 9414 | OG1 | THR | 1392 | −24.790 | −10.043 | 58.052 | 1.00 | 24.75 |
| ATOM | 9415 | CG2 | THR | 1392 | −26.263 | −11.842 | 57.424 | 1.00 | 21.60 |
| ATOM | 9416 | C | THR | 1392 | −28.133 | −9.844 | 56.309 | 1.00 | 25.62 |
| ATOM | 9417 | O | THR | 1392 | −28.371 | −10.658 | 55.411 | 1.00 | 24.15 |
| ATOM | 9418 | N | GLU | 1393 | −29.095 | −9.253 | 57.013 | 1.00 | 25.81 |
| ATOM | 9419 | CA | GLU | 1393 | −30.505 | −9.540 | 56.754 | 1.00 | 28.50 |
| ATOM | 9420 | CB | GLU | 1393 | −31.358 | −9.159 | 57.966 | 1.00 | 30.65 |
| ATOM | 9421 | CG | GLU | 1393 | −31.271 | −10.140 | 59.124 | 1.00 | 35.02 |
| ATOM | 9422 | CD | GLU | 1393 | −32.129 | −9.716 | 60.300 | 1.00 | 38.65 |
| ATOM | 9423 | OE1 | GLU | 1393 | −33.335 | −9.449 | 60.089 | 1.00 | 40.37 |
| ATOM | 9424 | OE2 | GLU | 1393 | −31.602 | −9.652 | 61.435 | 1.00 | 39.76 |
| ATOM | 9425 | C | GLU | 1393 | −31.038 | −8.818 | 55.520 | 1.00 | 27.06 |
| ATOM | 9426 | O | GLU | 1393 | −31.907 | −9.337 | 54.820 | 1.00 | 28.64 |
| ATOM | 9427 | N | ALA | 1394 | −30.515 | −7.627 | 55.254 | 1.00 | 24.47 |
| ATOM | 9428 | CA | ALA | 1394 | −30.961 | −6.847 | 54.110 | 1.00 | 24.04 |
| ATOM | 9429 | CB | ALA | 1394 | −30.508 | −5.394 | 54.265 | 1.00 | 22.66 |
| ATOM | 9430 | C | ALA | 1394 | −30.467 | −7.411 | 52.782 | 1.00 | 24.13 |
| ATOM | 9431 | O | ALA | 1394 | −31.165 | −7.322 | 51.768 | 1.00 | 22.55 |
| ATOM | 9432 | N | LEU | 1395 | −29.270 | −7.995 | 52.790 | 1.00 | 23.16 |
| ATOM | 9433 | CA | LEU | 1395 | −28.682 | −8.551 | 51.571 | 1.00 | 22.35 |
| ATOM | 9434 | CB | LEU | 1395 | −27.169 | −8.307 | 51.557 | 1.00 | 23.06 |
| ATOM | 9435 | CG | LEU | 1395 | −26.678 | −6.859 | 51.608 | 1.00 | 24.33 |
| ATOM | 9436 | CD1 | LEU | 1395 | −25.165 | −6.854 | 51.357 | 1.00 | 23.88 |
| ATOM | 9437 | CD2 | LEU | 1395 | −27.386 | −6.016 | 50.579 | 1.00 | 26.78 |
| ATOM | 9438 | C | LEU | 1395 | −28.939 | −10.036 | 51.352 | 1.00 | 20.89 |
| ATOM | 9439 | O | LEU | 1395 | −28.995 | −10.815 | 52.298 | 1.00 | 20.06 |
| ATOM | 9440 | N | ALA | 1396 | −29.091 | −10.416 | 50.085 | 1.00 | 20.42 |
| ATOM | 9441 | CA | ALA | 1396 | −29.330 | −11.805 | 49.716 | 1.00 | 21.29 |
| ATOM | 9442 | CB | ALA | 1396 | −30.126 | −11.871 | 48.413 | 1.00 | 19.07 |
| ATOM | 9443 | C | ALA | 1396 | −28.005 | −12.548 | 49.552 | 1.00 | 20.66 |
| ATOM | 9444 | O | ALA | 1396 | −27.916 | −13.753 | 49.809 | 1.00 | 19.21 |
| ATOM | 9445 | N | ILE | 1397 | −26.976 | −11.823 | 49.116 | 1.00 | 19.78 |
| ATOM | 9446 | CA | ILE | 1397 | −25.650 | −12.405 | 48.933 | 1.00 | 20.20 |
| ATOM | 9447 | CB | ILE | 1397 | −24.721 | −11.463 | 48.134 | 1.00 | 19.10 |
| ATOM | 9448 | CG2 | ILE | 1397 | −25.280 | −11.237 | 46.742 | 1.00 | 19.43 |
| ATOM | 9449 | CG1 | ILE | 1397 | −24.561 | −10.139 | 48.875 | 1.00 | 17.71 |
| ATOM | 9450 | CD1 | ILE | 1397 | −23.568 | −9.181 | 48.227 | 1.00 | 18.82 |
| ATOM | 9451 | C | ILE | 1397 | −25.020 | −12.649 | 50.299 | 1.00 | 20.56 |
| ATOM | 9452 | O | ILE | 1397 | −25.389 | −12.008 | 51.282 | 1.00 | 21.78 |
| ATOM | 9453 | N | PRO | 1398 | −24.066 | −13.586 | 50.381 | 1.00 | 22.06 |
| ATOM | 9454 | CD | PRO | 1398 | −23.622 | −14.529 | 49.342 | 1.00 | 22.24 |
| ATOM | 9455 | CA | PRO | 1398 | −23.419 | −13.871 | 51.661 | 1.00 | 22.34 |
| ATOM | 9456 | CB | PRO | 1398 | −22.633 | −15.150 | 51.373 | 1.00 | 23.09 |
| ATOM | 9457 | CG | PRO | 1398 | −22.353 | −15.057 | 49.917 | 1.00 | 26.50 |
| ATOM | 9458 | C | PRO | 1398 | −22.546 | −12.728 | 52.168 | 1.00 | 21.87 |
| ATOM | 9459 | O | PRO | 1398 | −21.840 | −12.075 | 51.394 | 1.00 | 21.73 |
| ATOM | 9460 | N | VAL | 1399 | −22.621 | −12.479 | 53.472 | 1.00 | 21.04 |
| ATOM | 9461 | CA | VAL | 1399 | −21.845 | −11.423 | 54.102 | 1.00 | 20.62 |
| ATOM | 9462 | CB | VAL | 1399 | −22.750 | −10.465 | 54.918 | 1.00 | 21.23 |
| ATOM | 9463 | CG1 | VAL | 1399 | −21.906 | −9.373 | 55.569 | 1.00 | 20.53 |
| ATOM | 9464 | CG2 | VAL | 1399 | −23.796 | −9.833 | 54.007 | 1.00 | 21.96 |
| ATOM | 9465 | C | VAL | 1399 | −20.793 | −12.033 | 55.021 | 1.00 | 20.44 |
| ATOM | 9466 | O | VAL | 1399 | −21.115 | −12.798 | 55.938 | 1.00 | 19.55 |
| ATOM | 9467 | N | ILE | 1400 | −19.536 | −11.694 | 54.751 | 1.00 | 20.90 |
| ATOM | 9468 | CA | ILE | 1400 | −18.392 | −12.181 | 55.522 | 1.00 | 19.21 |
| ATOM | 9469 | CB | ILE | 1400 | −17.218 | −12.559 | 54.590 | 1.00 | 20.36 |
| ATOM | 9470 | CG2 | ILE | 1400 | −16.008 | −12.966 | 55.418 | 1.00 | 20.56 |
| ATOM | 9471 | CG1 | ILE | 1400 | −17.643 | −13.705 | 53.668 | 1.00 | 22.22 |
| ATOM | 9472 | CD1 | ILE | 1400 | −16.590 | −14.082 | 52.643 | 1.00 | 24.06 |
| ATOM | 9473 | C | ILE | 1400 | −17.924 | −11.083 | 56.462 | 1.00 | 19.24 |
| ATOM | 9474 | O | ILE | 1400 | −17.585 | −9.985 | 56.026 | 1.00 | 17.97 |
| ATOM | 9475 | N | GLY | 1401 | −17.898 | −11.380 | 57.754 | 1.00 | 17.48 |
| ATOM | 9476 | CA | GLY | 1401 | −17.484 | −10.368 | 58.700 | 1.00 | 17.76 |
| ATOM | 9477 | C | GLY | 1401 | −16.182 | −10.615 | 59.429 | 1.00 | 17.82 |
| ATOM | 9478 | O | GLY | 1401 | −15.731 | −11.746 | 59.583 | 1.00 | 18.37 |
| ATOM | 9479 | N | ILE | 1402 | −15.567 | −9.519 | 59.846 | 1.00 | 17.14 |
| ATOM | 9480 | CA | ILE | 1402 | −14.342 | −9.549 | 60.622 | 1.00 | 19.20 |
| ATOM | 9481 | CB | ILE | 1402 | −13.061 | −9.437 | 59.731 | 1.00 | 17.10 |
| ATOM | 9482 | CG2 | ILE | 1402 | −13.207 | −8.319 | 58.708 | 1.00 | 19.67 |
| ATOM | 9483 | CG1 | ILE | 1402 | −11.844 | −9.222 | 60.626 | 1.00 | 22.25 |
| ATOM | 9484 | CD1 | ILE | 1402 | −10.521 | −9.488 | 59.939 | 1.00 | 21.22 |
| ATOM | 9485 | C | ILE | 1402 | −14.477 | −8.359 | 61.562 | 1.00 | 17.66 |
| ATOM | 9486 | O | ILE | 1402 | −14.489 | −7.202 | 61.134 | 1.00 | 21.00 |
| ATOM | 9487 | N | GLY | 1403 | −14.616 | −8.646 | 62.849 | 1.00 | 18.73 |
| ATOM | 9488 | CA | GLY | 1403 | −14.800 | −7.572 | 63.805 | 1.00 | 19.62 |
| ATOM | 9489 | C | GLY | 1403 | −16.208 | −7.017 | 63.675 | 1.00 | 20.89 |
| ATOM | 9490 | O | GLY | 1403 | −16.457 | −5.837 | 63.934 | 1.00 | 20.69 |
| ATOM | 9491 | N | ALA | 1404 | −17.137 | −7.871 | 63.259 | 1.00 | 20.95 |
| ATOM | 9492 | CA | ALA | 1404 | −18.529 | −7.452 | 63.100 | 1.00 | 22.83 |
| ATOM | 9493 | CB | ALA | 1404 | −18.881 | −7.354 | 61.614 | 1.00 | 24.62 |
| ATOM | 9494 | C | ALA | 1404 | −19.502 | −8.391 | 63.807 | 1.00 | 24.35 |
| ATOM | 9495 | O | ALA | 1404 | −20.706 | −8.344 | 63.558 | 1.00 | 24.70 |
| ATOM | 9496 | N | GLY | 1405 | −18.980 | −9.246 | 64.685 | 1.00 | 24.62 |
| ATOM | 9497 | CA | GLY | 1405 | −19.833 | −10.170 | 65.411 | 1.00 | 25.54 |
| ATOM | 9498 | C | GLY | 1405 | −20.075 | −11.478 | 64.683 | 1.00 | 26.30 |
| ATOM | 9499 | O | GLY | 1405 | −19.573 | −11.681 | 63.581 | 1.00 | 27.10 |
| ATOM | 9500 | N | ASN | 1406 | −20.854 | −12.367 | 65.297 | 1.00 | 25.69 |
| ATOM | 9501 | CA | ASN | 1406 | −21.149 | −13.670 | 64.701 | 1.00 | 25.78 |
| ATOM | 9502 | CB | ASN | 1406 | −21.144 | −14.756 | 65.781 | 1.00 | 26.59 |
| ATOM | 9503 | CG | ASN | 1406 | −22.323 | −14.641 | 66.745 | 1.00 | 28.85 |
| ATOM | 9504 | OD1 | ASN | 1406 | −22.638 | −15.586 | 67.470 | 1.00 | 29.88 |
| ATOM | 9505 | ND2 | ASN | 1406 | −22.972 | −13.481 | 66.760 | 1.00 | 25.54 |
| ATOM | 9506 | C | ASN | 1406 | −22.481 | −13.713 | 63.952 | 1.00 | 24.77 |
| ATOM | 9507 | O | ASN | 1406 | −22.956 | −14.787 | 63.579 | 1.00 | 25.44 |
| ATOM | 9508 | N | VAL | 1407 | −23.071 | −12.541 | 63.725 | 1.00 | 24.74 |
| ATOM | 9509 | CA | VAL | 1407 | −24.354 | −12.434 | 63.033 | 1.00 | 25.37 |
| ATOM | 9510 | CB | VAL | 1407 | −25.064 | −11.118 | 63.402 | 1.00 | 25.71 |
| ATOM | 9511 | CG1 | VAL | 1407 | −26.505 | −11.144 | 62.893 | 1.00 | 31.10 |
| ATOM | 9512 | CG2 | VAL | 1407 | −25.040 | −10.917 | 64.913 | 1.00 | 27.35 |
| ATOM | 9513 | C | VAL | 1407 | −24.224 | −12.497 | 61.512 | 1.00 | 25.31 |
| ATOM | 9514 | O | VAL | 1407 | −25.223 | −12.591 | 60.790 | 1.00 | 24.78 |
| ATOM | 9515 | N | THR | 1408 | −22.991 | −12.437 | 61.024 | 1.00 | 23.39 |
| ATOM | 9516 | CA | THR | 1408 | −22.753 | −12.490 | 59.591 | 1.00 | 21.03 |
| ATOM | 9517 | CB | THR | 1408 | −21.375 | −11.874 | 59.238 | 1.00 | 20.01 |
| ATOM | 9518 | OG1 | THR | 1408 | −20.354 | −12.456 | 60.064 | 1.00 | 19.12 |
| ATOM | 9519 | CG2 | THR | 1408 | −21.401 | −10.382 | 59.460 | 1.00 | 17.17 |
| ATOM | 9520 | C | THR | 1408 | −22.813 | −13.927 | 59.104 | 1.00 | 21.73 |
| ATOM | 9521 | O | THR | 1408 | −22.821 | −14.867 | 59.908 | 1.00 | 24.16 |
| ATOM | 9522 | N | ASP | 1409 | −22.874 | −14.090 | 57.785 | 1.00 | 19.78 |
| ATOM | 9523 | CA | ASP | 1409 | −22.942 | −15.406 | 57.157 | 1.00 | 19.91 |
| ATOM | 9524 | CB | ASP | 1409 | −23.371 | −15.268 | 55.696 | 1.00 | 21.36 |
| ATOM | 9525 | CG | ASP | 1409 | −24.712 | −14.600 | 55.553 | 1.00 | 22.44 |
| ATOM | 9526 | OD1 | ASP | 1409 | −25.693 | −15.136 | 56.107 | 1.00 | 22.97 |
| ATOM | 9527 | OD2 | ASP | 1409 | −24.786 | −13.536 | 54.899 | 1.00 | 25.06 |
| ATOM | 9528 | C | ASP | 1409 | −21.600 | −16.112 | 57.211 | 1.00 | 19.51 |
| ATOM | 9529 | O | ASP | 1409 | −21.533 | −17.342 | 57.236 | 1.00 | 18.44 |
| ATOM | 9530 | N | GLY | 1410 | −20.534 | −15.323 | 57.219 | 1.00 | 21.88 |
| ATOM | 9531 | CA | GLY | 1410 | −19.204 | −15.899 | 57.269 | 1.00 | 20.44 |
| ATOM | 9532 | C | GLY | 1410 | −18.249 | −15.097 | 58.124 | 1.00 | 19.85 |
| ATOM | 9533 | O | GLY | 1410 | −18.589 | −14.013 | 58.594 | 1.00 | 18.97 |
| ATOM | 9534 | N | GLN | 1411 | −17.050 | −15.643 | 58.322 | 1.00 | 19.88 |
| ATOM | 9535 | CA | GLN | 1411 | −16.011 | −15.002 | 59.116 | 1.00 | 20.00 |
| ATOM | 9536 | CB | GLN | 1411 | −15.817 | −15.730 | 60.447 | 1.00 | 19.91 |
| ATOM | 9537 | CG | GLN | 1411 | −16.981 | −15.636 | 61.417 | 1.00 | 19.40 |
| ATOM | 9538 | CD | GLN | 1411 | −17.301 | −14.209 | 61.801 | 1.00 | 16.91 |
| ATOM | 9539 | OE1 | GLN | 1411 | −16.414 | −13.434 | 62.153 | 1.00 | 18.73 |
| ATOM | 9540 | NE2 | GLN | 1411 | −18.578 | −13.859 | 61.752 | 1.00 | 18.43 |
| ATOM | 9541 | C | GLN | 1411 | −14.676 | −15.014 | 58.382 | 1.00 | 20.57 |
| ATOM | 9542 | O | GLN | 1411 | −14.421 | −15.889 | 57.558 | 1.00 | 19.66 |
| ATOM | 9543 | N | ILE | 1412 | −13.824 | −14.046 | 58.697 | 1.00 | 22.26 |
| ATOM | 9544 | CA | ILE | 1412 | −12.501 | −13.973 | 58.097 | 1.00 | 23.53 |
| ATOM | 9545 | CB | ILE | 1412 | −12.530 | −13.154 | 56.776 | 1.00 | 23.95 |
| ATOM | 9546 | CG2 | ILE | 1412 | −12.737 | −11.675 | 57.075 | 1.00 | 24.56 |
| ATOM | 9547 | CG1 | ILE | 1412 | −11.227 | −13.360 | 56.010 | 1.00 | 25.80 |
| ATOM | 9548 | CD1 | ILE | 1412 | −11.316 | −12.973 | 54.543 | 1.00 | 25.24 |
| ATOM | 9549 | C | ILE | 1412 | −11.536 | −13.335 | 59.097 | 1.00 | 25.52 |
| ATOM | 9550 | O | ILE | 1412 | −11.952 | −12.627 | 60.014 | 1.00 | 23.69 |
| ATOM | 9551 | N | LEU | 1413 | −10.248 | −13.607 | 58.933 | 1.00 | 27.48 |
| ATOM | 9552 | CA | LEU | 1413 | −9.243 | −13.032 | 59.815 | 1.00 | 28.85 |
| ATOM | 9553 | CB | LEU | 1413 | −9.351 | −13.635 | 61.216 | 1.00 | 34.02 |
| ATOM | 9554 | CG | LEU | 1413 | −9.299 | −12.665 | 62.403 | 1.00 | 37.41 |
| ATOM | 9555 | CD1 | LEU | 1413 | −9.200 | −13.472 | 63.680 | 1.00 | 39.43 |
| ATOM | 9556 | CD2 | LEU | 1413 | −8.100 | −11.718 | 62.283 | 1.00 | 39.02 |
| ATOM | 9557 | C | LEU | 1413 | −7.863 | −13.315 | 59.243 | 1.00 | 28.57 |
| ATOM | 9558 | O | LEU | 1413 | −7.692 | −14.259 | 58.470 | 1.00 | 25.03 |
| ATOM | 9559 | N | VAL | 1414 | −6.890 | −12.485 | 59.610 | 1.00 | 29.11 |
| ATOM | 9560 | CA | VAL | 1414 | −5.520 | −12.662 | 59.147 | 1.00 | 29.08 |
| ATOM | 9561 | CB | VAL | 1414 | −4.638 | −11.435 | 59.483 | 1.00 | 30.55 |
| ATOM | 9562 | CG1 | VAL | 1414 | −3.238 | −11.628 | 58.900 | 1.00 | 31.68 |
| ATOM | 9563 | CG2 | VAL | 1414 | −5.277 | −10.165 | 58.928 | 1.00 | 31.09 |
| ATOM | 9564 | C | VAL | 1414 | −4.961 | −13.880 | 59.863 | 1.00 | 28.58 |
| ATOM | 9565 | O | VAL | 1414 | −4.936 | −13.928 | 61.092 | 1.00 | 28.63 |
| ATOM | 9566 | N | MET | 1415 | −4.525 | −14.865 | 59.087 | 1.00 | 26.58 |
| ATOM | 9567 | CA | MET | 1415 | −3.976 | −16.096 | 59.644 | 1.00 | 25.18 |
| ATOM | 9568 | CB | MET | 1415 | −3.386 | −16.959 | 58.527 | 1.00 | 21.68 |
| ATOM | 9569 | CG | MET | 1415 | −2.253 | −16.306 | 57.777 | 1.00 | 20.54 |
| ATOM | 9570 | SD | MET | 1415 | −1.043 | −17.545 | 57.300 | 1.00 | 21.74 |
| ATOM | 9571 | CE | MET | 1415 | −0.091 | −17.623 | 58.832 | 1.00 | 21.73 |
| ATOM | 9572 | C | MET | 1415 | −2.912 | −15.850 | 60.713 | 1.00 | 25.50 |
| ATOM | 9573 | O | MET | 1415 | −2.793 | −16.614 | 61.671 | 1.00 | 25.46 |
| ATOM | 9574 | N | HIS | 1416 | −2.144 | −14.778 | 60.548 | 1.00 | 25.98 |
| ATOM | 9575 | CA | HIS | 1416 | −1.089 | −14.440 | 61.496 | 1.00 | 27.03 |
| ATOM | 9576 | CB | HIS | 1416 | −0.294 | −13.242 | 60.974 | 1.00 | 26.14 |
| ATOM | 9577 | CG | HIS | 1416 | 0.482 | −13.542 | 59.726 | 1.00 | 26.99 |
| ATOM | 9578 | CD2 | HIS | 1416 | 0.121 | −13.513 | 58.421 | 1.00 | 25.19 |
| ATOM | 9579 | ND1 | HIS | 1416 | 1.782 | −14.002 | 59.750 | 1.00 | 25.73 |
| ATOM | 9580 | CE1 | HIS | 1416 | 2.188 | −14.243 | 58.517 | 1.00 | 24.78 |
| ATOM | 9581 | NE2 | HIS | 1416 | 1.198 | −13.954 | 57.690 | 1.00 | 27.44 |
| ATOM | 9582 | C | HIS | 1416 | −1.651 | −14.163 | 62.889 | 1.00 | 28.75 |
| ATOM | 9583 | O | HIS | 1416 | −1.076 | −14.598 | 63.887 | 1.00 | 27.94 |
| ATOM | 9584 | N | ASP | 1417 | −2.773 | −13.449 | 62.966 | 1.00 | 29.75 |
| ATOM | 9585 | CA | ASP | 1417 | −3.386 | −13.156 | 64.262 | 1.00 | 33.00 |
| ATOM | 9586 | CB | ASP | 1417 | −4.458 | −12.069 | 64.134 | 1.00 | 34.18 |
| ATOM | 9587 | CG | ASP | 1417 | −3.886 | −10.733 | 63.731 | 1.00 | 37.67 |
| ATOM | 9588 | OD1 | ASP | 1417 | −2.900 | −10.292 | 64.361 | 1.00 | 39.25 |
| ATOM | 9589 | OD2 | ASP | 1417 | −4.429 | −10.114 | 62.788 | 1.00 | 40.11 |
| ATOM | 9590 | C | ASP | 1417 | −4.025 | −14.408 | 64.848 | 1.00 | 33.09 |
| ATOM | 9591 | O | ASP | 1417 | −4.130 | −14.549 | 66.067 | 1.00 | 34.61 |
| ATOM | 9592 | N | ALA | 1418 | −4.455 | −15.312 | 63.974 | 1.00 | 34.01 |
| ATOM | 9593 | CA | ALA | 1418 | −5.094 | −16.552 | 64.399 | 1.00 | 35.06 |
| ATOM | 9594 | CB | ALA | 1418 | −5.800 | −17.207 | 63.208 | 1.00 | 34.52 |
| ATOM | 9595 | C | ALA | 1418 | −4.110 | −17.535 | 65.034 | 1.00 | 36.11 |
| ATOM | 9596 | O | ALA | 1418 | −4.524 | −18.505 | 65.671 | 1.00 | 36.84 |
| ATOM | 9597 | N | PHE | 1419 | −2.813 | −17.287 | 64.863 | 1.00 | 36.18 |
| ATOM | 9598 | CA | PHE | 1419 | −1.791 | −18.161 | 65.436 | 1.00 | 36.22 |
| ATOM | 9599 | CB | PHE | 1419 | −0.999 | −18.849 | 64.322 | 1.00 | 37.87 |
| ATOM | 9600 | CG | PHE | 1419 | −1.861 | −19.607 | 63.349 | 1.00 | 39.07 |
| ATOM | 9601 | CD1 | PHE | 1419 | −2.859 | −20.465 | 63.806 | 1.00 | 38.80 |
| ATOM | 9602 | CD2 | PHE | 1419 | −1.671 | −19.472 | 61.978 | 1.00 | 39.70 |
| ATOM | 9603 | CE1 | PHE | 1419 | −3.654 | −21.176 | 62.914 | 1.00 | 39.60 |
| ATOM | 9604 | CE2 | PHE | 1419 | −2.461 | −20.180 | 61.075 | 1.00 | 39.92 |
| ATOM | 9605 | CZ | PHE | 1419 | −3.456 | −21.035 | 61.546 | 1.00 | 40.35 |
| ATOM | 9606 | C | PHE | 1419 | −0.838 | −17.412 | 66.369 | 1.00 | 35.97 |
| ATOM | 9607 | O | PHE | 1419 | 0.267 | −17.880 | 66.660 | 1.00 | 35.74 |
| ATOM | 9608 | N | GLY | 1420 | −1.278 | −16.245 | 66.832 | 1.00 | 35.56 |
| ATOM | 9609 | CA | GLY | 1420 | −0.472 | −15.444 | 67.739 | 1.00 | 34.95 |
| ATOM | 9610 | C | GLY | 1420 | 0.900 | −15.079 | 67.205 | 1.00 | 34.01 |
| ATOM | 9611 | O | GLY | 1420 | 1.762 | −14.622 | 67.960 | 1.00 | 33.90 |
| ATOM | 9612 | N | ILE | 1421 | 1.112 | −15.278 | 65.908 | 1.00 | 31.41 |
| ATOM | 9613 | CA | ILE | 1421 | 2.394 | −14.950 | 65.299 | 1.00 | 28.98 |
| ATOM | 9614 | CB | ILE | 1421 | 2.423 | −15.367 | 63.804 | 1.00 | 28.18 |
| ATOM | 9615 | CG2 | ILE | 1421 | 3.732 | −14.925 | 63.156 | 1.00 | 24.57 |
| ATOM | 9616 | CG1 | ILE | 1421 | 2.264 | −16.880 | 63.689 | 1.00 | 26.51 |
| ATOM | 9617 | CD1 | ILE | 1421 | 2.034 | −17.371 | 62.278 | 1.00 | 26.39 |
| ATOM | 9618 | C | ILE | 1421 | 2.626 | −13.447 | 65.411 | 1.00 | 29.18 |
| ATOM | 9619 | O | ILE | 1421 | 3.716 | −12.999 | 65.761 | 1.00 | 27.59 |
| ATOM | 9620 | N | THR | 1422 | 1.577 | −12.676 | 65.143 | 1.00 | 30.56 |
| ATOM | 9621 | CA | THR | 1422 | 1.654 | −11.222 | 65.195 | 1.00 | 33.09 |
| ATOM | 9622 | CB | THR | 1422 | 0.315 | −10.585 | 64.796 | 1.00 | 34.07 |
| ATOM | 9623 | OG1 | THR | 1422 | −0.711 | −11.042 | 65.685 | 1.00 | 35.73 |
| ATOM | 9624 | CG2 | THR | 1422 | −0.045 | −10.961 | 63.372 | 1.00 | 31.21 |
| ATOM | 9625 | C | THR | 1422 | 2.055 | −10.658 | 66.552 | 1.00 | 35.46 |
| ATOM | 9626 | O | THR | 1422 | 1.761 | −11.245 | 67.594 | 1.00 | 33.19 |
| ATOM | 9627 | N | GLY | 1423 | 2.716 | −9.503 | 66.502 | 1.00 | 38.12 |
| ATOM | 9628 | CA | GLY | 1423 | 3.187 | −8.802 | 67.682 | 1.00 | 42.38 |
| ATOM | 9629 | C | GLY | 1423 | 2.958 | −9.491 | 69.005 | 1.00 | 44.65 |
| ATOM | 9630 | O | GLY | 1423 | 3.540 | −10.541 | 69.273 | 1.00 | 47.25 |
| ATOM | 9631 | N | GLY | 1424 | 2.113 | −8.894 | 69.838 | 1.00 | 45.54 |
| ATOM | 9632 | CA | GLY | 1424 | 1.826 | −9.478 | 71.133 | 1.00 | 46.73 |
| ATOM | 9633 | C | GLY | 1424 | 0.367 | −9.366 | 71.525 | 1.00 | 47.44 |
| ATOM | 9634 | O | GLY | 1424 | −0.242 | −10.347 | 71.953 | 1.00 | 48.03 |
| ATOM | 9635 | N | HIS | 1425 | −0.202 | −8.174 | 71.378 | 1.00 | 47.09 |
| ATOM | 9636 | CA | HIS | 1425 | −1.597 | −7.968 | 71.745 | 1.00 | 48.66 |
| ATOM | 9637 | CB | HIS | 1425 | −1.732 | −6.705 | 72.598 | 1.00 | 50.45 |
| ATOM | 9638 | CG | HIS | 1425 | −2.977 | −6.666 | 73.427 | 1.00 | 52.26 |
| ATOM | 9639 | CD2 | HIS | 1425 | −3.834 | −5.654 | 73.701 | 1.00 | 53.70 |
| ATOM | 9640 | ND1 | HIS | 1425 | −3.434 | −7.756 | 74.136 | 1.00 | 53.03 |
| ATOM | 9641 | CE1 | HIS | 1425 | −4.518 | −7.418 | 74.812 | 1.00 | 52.99 |
| ATOM | 9642 | NE2 | HIS | 1425 | −4.781 | −6.148 | 74.566 | 1.00 | 54.24 |
| ATOM | 9643 | C | HIS | 1425 | −2.485 | −7.868 | 70.511 | 1.00 | 47.46 |
| ATOM | 9644 | O | HIS | 1425 | −2.627 | −6.802 | 69.917 | 1.00 | 46.90 |
| ATOM | 9645 | N | ILE | 1426 | −3.078 | −8.993 | 70.129 | 1.00 | 47.14 |
| ATOM | 9646 | CA | ILE | 1426 | −3.942 | −9.039 | 68.960 | 1.00 | 46.99 |
| ATOM | 9647 | CB | ILE | 1426 | −4.374 | −10.485 | 68.648 | 1.00 | 47.44 |
| ATOM | 9648 | CG2 | ILE | 1426 | −3.160 | −11.313 | 68.259 | 1.00 | 49.26 |
| ATOM | 9649 | CG1 | ILE | 1426 | −5.069 | −11.098 | 69.860 | 1.00 | 47.01 |
| ATOM | 9650 | CD1 | ILE | 1426 | −5.604 | −12.486 | 69.606 | 1.00 | 47.10 |
| ATOM | 9651 | C | ILE | 1426 | −5.184 | −8.177 | 69.148 | 1.00 | 45.31 |
| ATOM | 9652 | O | ILE | 1426 | −5.532 | −7.801 | 70.271 | 1.00 | 45.25 |
| ATOM | 9653 | N | PRO | 1427 | 5.868 | 7.844 | 68.043 | 1.00 | 44.13 |
| ATOM | 9654 | CD | PRO | 1427 | −5.581 | −8.236 | 66.652 | 1.00 | 43.74 |
| ATOM | 9655 | CA | PRO | 1427 | −7.078 | −7.020 | 68.112 | 1.00 | 42.56 |
| ATOM | 9656 | CB | PRO | 1427 | −7.414 | −6.784 | 66.642 | 1.00 | 42.85 |
| ATOM | 9657 | CG | PRO | 1427 | −6.925 | −8.038 | 65.989 | 1.00 | 44.37 |
| ATOM | 9658 | C | PRO | 1427 | −8.212 | −7.703 | 68.870 | 1.00 | 40.69 |
| ATOM | 9659 | O | PRO | 1427 | −8.274 | −8.928 | 68.941 | 1.00 | 39.71 |
| ATOM | 9660 | N | LYS | 1428 | −9.105 | −6.899 | 69.436 | 1.00 | 39.55 |
| ATOM | 9661 | CA | LYS | 1428 | −10.238 | −7.422 | 70.189 | 1.00 | 38.02 |
| ATOM | 9662 | CB | LYS | 1428 | −11.155 | −6.274 | 70.631 | 1.00 | 40.74 |
| ATOM | 9663 | CG | LYS | 1428 | −10.693 | −5.483 | 71.860 | 1.00 | 44.43 |
| ATOM | 9664 | CD | LYS | 1428 | −9.402 | −4.698 | 71.629 | 1.00 | 46.69 |
| ATOM | 9665 | CE | LYS | 1428 | −8.168 | −5.494 | 72.046 | 1.00 | 47.27 |
| ATOM | 9666 | NZ | LYS | 1428 | −6.917 | −4.715 | 71.829 | 1.00 | 48.13 |
| ATOM | 9667 | C | LYS | 1428 | −11.065 | −8.451 | 69.418 | 1.00 | 35.07 |
| ATOM | 9668 | O | LYS | 1428 | −11.557 | −9.417 | 69.999 | 1.00 | 33.86 |
| ATOM | 9669 | N | PHE | 1429 | −11.213 | −8.250 | 68.112 | 1.00 | 32.23 |
| ATOM | 9670 | CA | PHE | 1429 | −12.014 | −9.159 | 67.293 | 1.00 | 28.78 |
| ATOM | 9671 | CB | PHE | 1429 | −12.484 | −8.430 | 66.027 | 1.00 | 27.62 |
| ATOM | 9672 | CG | PHE | 1429 | −11.366 | −7.984 | 65.131 | 1.00 | 24.25 |
| ATOM | 9673 | CD1 | PHE | 1429 | −10.713 | −8.895 | 64.304 | 1.00 | 23.47 |
| ATOM | 9674 | CD2 | PHE | 1429 | −10.962 | −6.654 | 65.116 | 1.00 | 22.84 |
| ATOM | 9675 | CE1 | PHE | 1429 | −9.673 | −8.489 | 63.476 | 1.00 | 21.56 |
| ATOM | 9676 | CE2 | PHE | 1429 | −9.922 | −6.237 | 64.293 | 1.00 | 23.95 |
| ATOM | 9677 | CZ | PHE | 1429 | −9.276 | −7.156 | 63.469 | 1.00 | 24.89 |
| ATOM | 9678 | C | PHE | 1429 | −11.314 | −10.463 | 66.914 | 1.00 | 28.44 |
| ATOM | 9679 | O | PHE | 1429 | −11.952 | −11.388 | 66.424 | 1.00 | 26.30 |
| ATOM | 9680 | N | ALA | 1430 | −10.008 | −10.534 | 67.149 | 1.00 | 28.50 |
| ATOM | 9681 | CA | ALA | 1430 | −9.236 | −11.726 | 66.816 | 1.00 | 29.62 |
| ATOM | 9682 | CB | ALA | 1430 | −7.854 | −11.325 | 66.325 | 1.00 | 29.24 |
| ATOM | 9683 | C | ALA | 1430 | −9.106 | −12.685 | 67.998 | 1.00 | 31.64 |
| ATOM | 9684 | O | ALA | 1430 | −9.332 | −12.311 | 69.150 | 1.00 | 31.63 |
| ATOM | 9685 | N | LYS | 1431 | −8.741 | −13.927 | 67.695 | 1.00 | 31.81 |
| ATOM | 9686 | CA | LYS | 1431 | −8.561 | −14.949 | 68.712 | 1.00 | 32.80 |
| ATOM | 9687 | CB | LYS | 1431 | −9.847 | −15.759 | 68.891 | 1.00 | 33.38 |
| ATOM | 9688 | CG | LYS | 1431 | −9.731 | −16.878 | 69.916 | 1.00 | 35.77 |
| ATOM | 9689 | CD | LYS | 1431 | −11.042 | −17.633 | 70.055 | 1.00 | 36.57 |
| ATOM | 9690 | CE | LYS | 1431 | −10.902 | −18.805 | 70.998 | 1.00 | 37.40 |
| ATOM | 9691 | NZ | LYS | 1431 | −12.189 | −19.531 | 71.174 | 1.00 | 38.32 |
| ATOM | 9692 | C | LYS | 1431 | −7.420 | −15.886 | 68.331 | 1.00 | 33.12 |
| ATOM | 9693 | O | LYS | 1431 | −7.379 | −16.414 | 67.213 | 1.00 | 32.77 |
| ATOM | 9694 | N | ASN | 1432 | −6.496 | −16.085 | 69.266 | 1.00 | 31.72 |
| ATOM | 9695 | CA | ASN | 1432 | −5.350 | −16.962 | 69.054 | 1.00 | 30.83 |
| ATOM | 9696 | CB | ASN | 1432 | −4.260 | −16.655 | 70.087 | 1.00 | 29.88 |
| ATOM | 9697 | CG | ASN | 1432 | −2.995 | −17.463 | 69.860 | 1.00 | 28.49 |
| ATOM | 9698 | OD1 | ASN | 1432 | −3.046 | −18.600 | 69.378 | 1.00 | 27.87 |
| ATOM | 9699 | ND2 | ASN | 1432 | −1.856 | −16.889 | 70.223 | 1.00 | 26.41 |
| ATOM | 9700 | C | ASN | 1432 | −5.814 | −18.403 | 69.225 | 1.00 | 30.31 |
| ATOM | 9701 | O | ASN | 1432 | −5.917 | −18.893 | 70.347 | 1.00 | 32.09 |
| ATOM | 9702 | N | PHE | 1433 | −6.095 | −19.078 | 68.115 | 1.00 | 30.31 |
| ATOM | 9703 | CA | PHE | 1433 | −6.553 | −20.463 | 68.164 | 1.00 | 30.25 |
| ATOM | 9704 | CB | PHE | 1433 | −7.293 | −20.840 | 66.878 | 1.00 | 31.29 |
| ATOM | 9705 | CG | PHE | 1433 | −8.609 | −20.144 | 66.706 | 1.00 | 32.82 |
| ATOM | 9706 | CD1 | PHE | 1433 | −8.682 | −18.899 | 66.095 | 1.00 | 32.79 |
| ATOM | 9707 | CD2 | PHE | 1433 | −9.781 | −20.737 | 67.160 | 1.00 | 33.56 |
| ATOM | 9708 | CE1 | PHE | 1433 | −9.907 | −18.254 | 65.936 | 1.00 | 34.13 |
| ATOM | 9709 | CE2 | PHE | 1433 | −11.007 | −20.102 | 67.007 | 1.00 | 34.37 |
| ATOM | 9710 | CZ | PHE | 1433 | −11.072 | −18.858 | 66.394 | 1.00 | 34.19 |
| ATOM | 9711 | C | PHE | 1433 | −5.415 | −21.449 | 68.383 | 1.00 | 30.90 |
| ATOM | 9712 | O | PHE | 1433 | −5.652 | −22.634 | 68.616 | 1.00 | 30.90 |
| ATOM | 9713 | N | LEU | 1434 | −4.179 | −20.969 | 68.293 | 1.00 | 31.92 |
| ATOM | 9714 | CA | LEU | 1434 | −3.034 | −21.847 | 68.501 | 1.00 | 33.45 |
| ATOM | 9715 | CB | LEU | 1434 | −1.783 | −21.276 | 67.834 | 1.00 | 30.12 |
| ATOM | 9716 | CG | LEU | 1434 | −0.507 | −22.124 | 67.974 | 1.00 | 28.48 |
| ATOM | 9717 | CD1 | LEU | 1434 | −0.732 | −23.515 | 67.385 | 1.00 | 24.04 |
| ATOM | 9718 | CD2 | LEU | 1434 | −0.646 | −21.422 | 67.263 | 1.00 | 29.29 |
| ATOM | 9719 | C | LEU | 1434 | −2.792 | −22.008 | 69.997 | 1.00 | 35.53 |
| ATOM | 9720 | O | LEU | 1434 | −2.528 | −23.109 | 70.472 | 1.00 | 35.29 |
| ATOM | 9721 | N | ALA | 1435 | −2.890 | −20.903 | 70.731 | 1.00 | 39.10 |
| ATOM | 9722 | CA | ALA | 1435 | −2.685 | −20.916 | 72.175 | 1.00 | 43.53 |
| ATOM | 9723 | CB | ALA | 1435 | −2.849 | −19.512 | 72.741 | 1.00 | 43.69 |
| ATOM | 9724 | C | ALA | 1435 | −3.675 | −21.864 | 72.833 | 1.00 | 46.59 |
| ATOM | 9725 | O | ALA | 1435 | −3.376 | −22.480 | 73.857 | 1.00 | 48.64 |
| ATOM | 9726 | N | GLU | 1436 | −4.857 | −21.974 | 72.235 | 1.00 | 49.35 |
| ATOM | 9727 | CA | GLU | 1436 | −5.908 | −22.852 | 72.742 | 1.00 | 52.29 |
| ATOM | 9728 | CB | GLU | 1436 | −7.250 | −22.507 | 72.085 | 1.00 | 54.27 |
| ATOM | 9729 | CG | GLU | 1436 | −7.670 | −21.046 | 72.213 | 1.00 | 57.48 |
| ATOM | 9730 | CD | GLU | 1436 | −8.111 | −20.674 | 73.617 | 1.00 | 59.19 |
| ATOM | 9731 | OE1 | GLU | 1436 | −7.296 | −20.800 | 74.557 | 1.00 | 60.49 |
| ATOM | 9732 | OE2 | GLU | 1436 | −9.278 | −20.251 | 73.779 | 1.00 | 59.69 |
| ATOM | 9733 | C | GLU | 1436 | −5.549 | −24.300 | 72.423 | 1.00 | 52.82 |
| ATOM | 9734 | O | GLU | 1436 | −6.359 | −25.208 | 72.610 | 1.00 | 53.16 |
| ATOM | 9735 | N | THR | 1437 | −4.334 | −24.504 | 71.926 | 1.00 | 53.46 |
| ATOM | 9736 | CA | THR | 1437 | −3.855 | −25.839 | 71.582 | 1.00 | 53.71 |
| ATOM | 9737 | CB | THR | 1437 | −4.497 | −26.343 | 70.268 | 1.00 | 54.29 |
| ATOM | 9738 | OG1 | THR | 1437 | −4.009 | −27.658 | 69.968 | 1.00 | 55.02 |
| ATOM | 9739 | CG2 | THR | 1437 | −4.167 | −25.405 | 69.113 | 1.00 | 55.13 |
| ATOM | 9740 | C | THR | 1437 | −2.332 | −25.849 | 71.440 | 1.00 | 53.32 |
| ATOM | 9741 | O | THR | 1437 | −1.632 | −25.088 | 72.113 | 1.00 | 53.43 |
| ATOM | 9742 | N | GLY | 1438 | −1.822 | −26.716 | 70.571 | 1.00 | 51.53 |
| ATOM | 9743 | CA | GLY | 1438 | −0.387 | −26.798 | 70.367 | 1.00 | 49.87 |
| ATOM | 9744 | C | GLY | 1438 | −0.031 | −27.262 | 68.970 | 1.00 | 48.04 |
| ATOM | 9745 | O | GLY | 1438 | 1.117 | −27.603 | 68.690 | 1.00 | 48.97 |
| ATOM | 9746 | N | ASP | 1439 | −1.023 | −27.265 | 68.088 | 1.00 | 45.31 |
| ATOM | 9747 | CA | ASP | 1439 | −0.826 | −27.699 | 66.714 | 1.00 | 42.62 |
| ATOM | 9748 | CB | ASP | 1439 | −1.358 | −29.129 | 66.553 | 1.00 | 44.51 |
| ATOM | 9749 | CG | ASP | 1439 | −1.261 | −29.633 | 65.132 | 1.00 | 46.55 |
| ATOM | 9750 | OD1 | ASP | 1439 | −2.119 | −29.256 | 64.309 | 1.00 | 47.77 |
| ATOM | 9751 | OD2 | ASP | 1439 | −0.319 | −30.402 | 64.836 | 1.00 | 48.59 |
| ATOM | 9752 | C | ASP | 1439 | −1.540 | −26.747 | 65.758 | 1.00 | 39.71 |
| ATOM | 9753 | O | ASP | 1439 | −2.752 | −26.552 | 65.850 | 1.00 | 38.19 |
| ATOM | 9754 | N | ILE | 1440 | −0.779 | −26.150 | 64.845 | 1.00 | 37.29 |
| ATOM | 9755 | CA | ILE | 1440 | −1.337 | −25.206 | 63.883 | 1.00 | 34.57 |
| ATOM | 9756 | CB | ILE | 1440 | −0.268 | −24.751 | 62.861 | 1.00 | 33.52 |
| ATOM | 9757 | CG2 | ILE | 1440 | −0.920 | −23.930 | 61.748 | 1.00 | 33.39 |
| ATOM | 9758 | CG1 | ILE | 1440 | 0.800 | −23.920 | 63.572 | 1.00 | 32.81 |
| ATOM | 9759 | CD1 | ILE | 1440 | 1.930 | −23.460 | 62.678 | 1.00 | 31.48 |
| ATOM | 9760 | C | ILE | 1440 | −2.540 | −25.774 | 63.138 | 1.00 | 32.97 |
| ATOM | 9761 | O | ILE | 1440 | −3.558 | −25.097 | 62.994 | 1.00 | 32.77 |
| ATOM | 9762 | N | ARG | 1441 | −2.429 | −27.012 | 62.668 | 1.00 | 31.92 |
| ATOM | 9763 | CA | ARG | 1441 | −3.532 | −27.631 | 61.947 | 1.00 | 30.40 |
| ATOM | 9764 | CB | ARG | 1441 | −3.114 | −28.993 | 61.389 | 1.00 | 30.10 |
| ATOM | 9765 | CG | ARG | 1441 | −2.154 | −28.897 | 60.205 | 1.00 | 31.94 |
| ATOM | 9766 | CD | ARG | 1441 | −1.662 | −30.266 | 59.761 | 1.00 | 31.51 |
| ATOM | 9767 | NE | ARG | 1441 | −0.801 | −30.192 | 58.580 | 1.00 | 31.83 |
| ATOM | 9768 | CZ | ARG | 1441 | 0.381 | −29.583 | 58.542 | 1.00 | 31.78 |
| ATOM | 9769 | NH1 | ARG | 1441 | 0.863 | −28.986 | 59.624 | 1.00 | 31.49 |
| ATOM | 9770 | NH2 | ARG | 1441 | 1.086 | −29.571 | 57.418 | 1.00 | 31.50 |
| ATOM | 9771 | C | ARG | 1441 | −4.730 | −27.776 | 62.869 | 1.00 | 30.29 |
| ATOM | 9772 | O | ARG | 1441 | −5.881 | −27.702 | 62.432 | 1.00 | 30.16 |
| ATOM | 9773 | N | ALA | 1442 | −4.459 | −27.979 | 64.155 | 1.00 | 29.42 |
| ATOM | 9774 | CA | ALA | 1442 | −5.527 | −28.115 | 65.133 | 1.00 | 28.31 |
| ATOM | 9775 | CB | ALA | 1442 | −4.963 | −28.620 | 66.469 | 1.00 | 27.62 |
| ATOM | 9776 | C | ALA | 1442 | −6.183 | −26.751 | 65.308 | 1.00 | 26.62 |
| ATOM | 9777 | O | ALA | 1442 | −7.396 | −26.656 | 65.471 | 1.00 | 28.00 |
| ATOM | 9778 | N | ALA | 1443 | −5.377 | −25.694 | 65.270 | 1.00 | 24.98 |
| ATOM | 9779 | CA | ALA | 1443 | −5.903 | −24.343 | 65.411 | 1.00 | 24.60 |
| ATOM | 9780 | CB | ALA | 1443 | −4.759 | −23.330 | 65.421 | 1.00 | 24.64 |
| ATOM | 9781 | C | ALA | 1443 | −6.871 | −24.043 | 64.264 | 1.00 | 24.55 |
| ATOM | 9782 | O | ALA | 1443 | −7.889 | −23.373 | 64.458 | 1.00 | 24.61 |
| ATOM | 9783 | N | VAL | 1444 | −6.553 | −24.547 | 63.072 | 1.00 | 24.06 |
| ATOM | 9784 | CA | VAL | 1444 | −7.396 | −24.344 | 61.895 | 1.00 | 25.36 |
| ATOM | 9785 | CB | VAL | 1444 | −6.738 | −24.933 | 60.620 | 1.00 | 25.58 |
| ATOM | 9786 | CG1 | VAL | 1444 | −7.681 | −24.782 | 59.430 | 1.00 | 26.78 |
| ATOM | 9787 | CG2 | VAL | 1444 | −5.423 | −24.217 | 60.338 | 1.00 | 24.41 |
| ATOM | 9788 | C | VAL | 1444 | −8.757 | −25.002 | 62.086 | 1.00 | 26.13 |
| ATOM | 9789 | O | VAL | 1444 | −9.792 | −24.378 | 61.854 | 1.00 | 25.38 |
| ATOM | 9790 | N | ARG | 1445 | −8.749 | −26.262 | 62.509 | 1.00 | 27.76 |
| ATOM | 9791 | CA | ARG | 1445 | −9.988 | −27.001 | 62.723 | 1.00 | 29.79 |
| ATOM | 9792 | CB | ARG | 1445 | −9.688 | −28.459 | 63.088 | 1.00 | 30.48 |
| ATOM | 9793 | CG | ARG | 1445 | −9.065 | −29.272 | 61.953 | 1.00 | 31.74 |
| ATOM | 9794 | CD | ARG | 1445 | −9.068 | −30.766 | 62.259 | 1.00 | 33.04 |
| ATOM | 9795 | NE | ARG | 1445 | −8.249 | −31.106 | 63.424 | 1.00 | 34.02 |
| ATOM | 9796 | CZ | ARG | 1445 | −6.941 | −31.341 | 63.383 | 1.00 | 33.14 |
| ATOM | 9797 | NH1 | ARG | 1445 | −6.286 | −31.278 | 62.231 | 1.00 | 31.44 |
| ATOM | 9798 | NH2 | ARG | 1445 | −6.288 | −31.650 | 64.495 | 1.00 | 33.53 |
| ATOM | 9799 | C | ARG | 1445 | −10.846 | −26.361 | 63.809 | 1.00 | 30.41 |
| ATOM | 9800 | O | ARG | 1445 | −12.070 | −26.298 | 63.685 | 1.00 | 29.87 |
| ATOM | 9801 | N | GLN | 1446 | −10.205 | −25.877 | 64.868 | 1.00 | 31.30 |
| ATOM | 9802 | CA | GLN | 1446 | −10.937 | −25.246 | 65.958 | 1.00 | 32.71 |
| ATOM | 9803 | CB | GLN | 1446 | −9.987 | −24.877 | 67.098 | 1.00 | 35.17 |
| ATOM | 9804 | CG | GLN | 1446 | −10.699 | −24.629 | 68.420 | 1.00 | 39.56 |
| ATOM | 9805 | CD | GLN | 1446 | −9.759 | −24.160 | 69.514 | 1.00 | 41.33 |
| ATOM | 9806 | OE1 | GLN | 1446 | −8.609 | −24.608 | 69.599 | 1.00 | 42.46 |
| ATOM | 9807 | NE2 | GLN | 1446 | −10.247 | −23.262 | 70.371 | 1.00 | 40.91 |
| ATOM | 9808 | C | GLN | 1446 | −11.632 | −23.988 | 65.440 | 1.00 | 31.61 |
| ATOM | 9809 | O | GLN | 1446 | −12.792 | −23.729 | 65.755 | 1.00 | 30.44 |
| ATOM | 9810 | N | TYR | 1447 | −10.907 | −23.211 | 64.643 | 1.00 | 31.24 |
| ATOM | 9811 | CA | TYR | 1447 | −11.444 | −21.981 | 64.072 | 1.00 | 30.17 |
| ATOM | 9812 | CB | TYR | 1447 | −10.361 | −21.286 | 63.242 | 1.00 | 29.43 |
| ATOM | 9813 | CG | TYR | 1447 | −10.865 | −20.155 | 62.374 | 1.00 | 28.31 |
| ATOM | 9814 | CD1 | TYR | 1447 | −11.525 | −19.059 | 62.927 | 1.00 | 27.71 |
| ATOM | 9815 | CE1 | TYR | 1447 | −11.999 | −18.023 | 62.122 | 1.00 | 25.98 |
| ATOM | 9816 | CD2 | TYR | 1447 | −10.689 | −20.190 | 60.990 | 1.00 | 28.41 |
| ATOM | 9817 | CE2 | TYR | 1447 | −11.158 | −19.164 | 60.179 | 1.00 | 26.24 |
| ATOM | 9818 | CZ | TYR | 1447 | −11.810 | −18.087 | 60.748 | 1.00 | 27.37 |
| ATOM | 9819 | OH | TYR | 1447 | −12.279 | −17.083 | 59.939 | 1.00 | 26.55 |
| ATOM | 9820 | C | TYR | 1447 | −12.679 | −22.267 | 63.213 | 1.00 | 29.65 |
| ATOM | 9821 | O | TYR | 1447 | −13.685 | −21.559 | 63.301 | 1.00 | 30.55 |
| ATOM | 9822 | N | MET | 1448 | −12.598 | −23.309 | 62.392 | 1.00 | 29.56 |
| ATOM | 9823 | CA | MET | 1448 | −13.703 | −23.702 | 61.521 | 1.00 | 30.34 |
| ATOM | 9824 | CB | MET | 1448 | −13.282 | −24.878 | 60.631 | 1.00 | 30.01 |
| ATOM | 9825 | CG | MET | 1448 | −12.224 | −24.529 | 59.580 | 1.00 | 31.10 |
| ATOM | 9826 | SD | MET | 1448 | −11.474 | −25.991 | 58.818 | 1.00 | 31.68 |
| ATOM | 9827 | CE | MET | 1448 | −12.791 | −26.527 | 57.742 | 1.00 | 29.90 |
| ATOM | 9828 | C | MET | 1448 | −14.935 | −24.103 | 62.327 | 1.00 | 30.96 |
| ATOM | 9829 | O | MET | 1448 | −16.045 | −23.636 | 62.061 | 1.00 | 29.66 |
| ATOM | 9830 | N | ALA | 1449 | −14.729 | −24.976 | 63.308 | 1.00 | 31.49 |
| ATOM | 9831 | CA | ALA | 1449 | −15.814 | −25.467 | 64.152 | 1.00 | 32.03 |
| ATOM | 9832 | CB | ALA | 1449 | −15.293 | −26.567 | 65.076 | 1.00 | 32.34 |
| ATOM | 9833 | C | ALA | 1449 | −16.485 | −24.369 | 64.974 | 1.00 | 32.12 |
| ATOM | 9834 | O | ALA | 1449 | −17.708 | −24.350 | 65.108 | 1.00 | 33.68 |
| ATOM | 9835 | N | GLU | 1450 | −15.692 | −23.460 | 65.529 | 1.00 | 32.10 |
| ATOM | 9836 | CA | GLU | 1450 | −16.253 | −22.382 | 66.335 | 1.00 | 33.21 |
| ATOM | 9837 | CB | GLU | 1450 | −15.154 | −21.685 | 67.141 | 1.00 | 34.06 |
| ATOM | 9838 | CG | GLU | 1450 | −14.691 | −22.482 | 68.353 | 1.00 | 36.22 |
| ATOM | 9839 | CD | GLU | 1450 | −13.733 | −21.708 | 69.231 | 1.00 | 38.09 |
| ATOM | 9840 | OE1 | GLU | 1450 | −14.030 | −20.539 | 69.549 | 1.00 | 40.42 |
| ATOM | 9841 | OE2 | GLU | 1450 | −12.685 | −22.269 | 69.615 | 1.00 | 41.16 |
| ATOM | 9842 | C | GLU | 1450 | −17.023 | −21.359 | 65.508 | 1.00 | 32.93 |
| ATOM | 9843 | O | GLU | 1450 | −17.956 | −20.731 | 66.010 | 1.00 | 31.71 |
| ATOM | 9844 | N | VAL | 1451 | −16.636 | −21.186 | 64.246 | 1.00 | 31.86 |
| ATOM | 9845 | CA | VAL | 1451 | −17.330 | −20.241 | 63.373 | 1.00 | 31.03 |
| ATOM | 9846 | CB | VAL | 1451 | −16.534 | −19.983 | 62.064 | 1.00 | 30.01 |
| ATOM | 9847 | CG1 | VAL | 1451 | −17.419 | −19.275 | 61.041 | 1.00 | 29.75 |
| ATOM | 9848 | CG2 | VAL | 1451 | −15.312 | −19.133 | 62.367 | 1.00 | 28.24 |
| ATOM | 9849 | C | VAL | 1451 | −18.713 | −20.783 | 63.017 | 1.00 | 31.90 |
| ATOM | 9850 | O | VAL | 1451 | −19.705 | −20.060 | 63.094 | 1.00 | 32.23 |
| ATOM | 9851 | N | GLU | 1452 | −18.766 | −22.059 | 62.639 | 1.00 | 31.88 |
| ATOM | 9852 | CA | GLU | 1452 | −20.019 | −22.704 | 62.262 | 1.00 | 33.73 |
| ATOM | 9853 | CB | GLU | 1452 | −19.741 | −24.083 | 61.660 | 1.00 | 34.97 |
| ATOM | 9854 | CG | GLU | 1452 | −20.993 | −24.838 | 61.243 | 1.00 | 38.84 |
| ATOM | 9855 | CD | GLU | 1452 | −20.681 | −26.118 | 60.490 | 1.00 | 41.04 |
| ATOM | 9856 | OE1 | GLU | 1452 | −21.632 | −26.820 | 60.088 | 1.00 | 42.35 |
| ATOM | 9857 | OE2 | GLU | 1452 | −19.483 | −26.421 | 60.296 | 1.00 | 43.66 |
| ATOM | 9858 | C | GLU | 1452 | −20.975 | −22.846 | 63.444 | 1.00 | 34.26 |
| ATOM | 9859 | O | GLU | 1452 | −22.194 | −22.815 | 63.274 | 1.00 | 33.56 |
| ATOM | 9860 | N | SER | 1453 | −20.412 | −22.996 | 64.638 | 1.00 | 34.47 |
| ATOM | 9861 | CA | SER | 1453 | −21.207 | −23.145 | 65.850 | 1.00 | 35.91 |
| ATOM | 9862 | CB | SER | 1453 | −20.414 | −23.918 | 66.903 | 1.00 | 36.48 |
| ATOM | 9863 | OG | SER | 1453 | −20.068 | −25.205 | 66.416 | 1.00 | 39.52 |
| ATOM | 9864 | C | SER | 1453 | −21.608 | −21.786 | 66.414 | 1.00 | 35.30 |
| ATOM | 9865 | O | SER | 1453 | −22.490 | −21.690 | 67.269 | 1.00 | 35.49 |
| ATOM | 9866 | N | GLY | 1454 | −20.959 | −20.737 | 65.929 | 1.00 | 33.14 |
| ATOM | 9867 | CA | GLY | 1454 | −21.267 | −19.405 | 66.407 | 1.00 | 32.40 |
| ATOM | 9868 | C | GLY | 1454 | −20.441 | −19.042 | 67.624 | 1.00 | 31.91 |
| ATOM | 9869 | O | GLY | 1454 | −20.491 | −17.906 | 68.100 | 1.00 | 31.65 |
| ATOM | 9870 | N | VAL | 1455 | −19.680 | −20.008 | 68.132 | 1.00 | 32.10 |
| ATOM | 9871 | CA | VAL | 1455 | −18.828 | −19.781 | 69.298 | 1.00 | 31.24 |
| ATOM | 9872 | CB | VAL | 1455 | −17.930 | −21.009 | 69.580 | 1.00 | 32.35 |
| ATOM | 9873 | CG1 | VAL | 1455 | −17.034 | −20.735 | 70.777 | 1.00 | 32.27 |
| ATOM | 9874 | CG2 | VAL | 1455 | −18.787 | −22.241 | 69.820 | 1.00 | 33.67 |
| ATOM | 9875 | C | VAL | 1455 | −17.939 | −18.570 | 69.033 | 1.00 | 30.78 |
| ATOM | 9876 | O | VAL | 1455 | −17.739 | −17.722 | 69.905 | 1.00 | 30.55 |
| ATOM | 9877 | N | TYR | 1456 | −17.410 | −18.500 | 67.815 | 1.00 | 28.80 |
| ATOM | 9878 | CA | TYR | 1456 | −16.545 | −17.401 | 67.405 | 1.00 | 27.94 |
| ATOM | 9879 | CB | TYR | 1456 | −15.157 | −17.935 | 67.012 | 1.00 | 26.12 |
| ATOM | 9880 | CG | TYR | 1456 | −14.226 | −16.846 | 66.531 | 1.00 | 23.34 |
| ATOM | 9881 | CD1 | TYR | 1456 | −13.651 | −15.950 | 67.433 | 1.00 | 22.92 |
| ATOM | 9882 | CE1 | TYR | 1456 | −12.873 | −14.883 | 66.995 | 1.00 | 22.74 |
| ATOM | 9883 | CD2 | TYR | 1456 | −13.989 | −16.652 | 65.169 | 1.00 | 24.91 |
| ATOM | 9884 | CE2 | TYR | 1456 | −13.212 | −15.585 | 64.718 | 1.00 | 22.93 |
| ATOM | 9885 | CZ | TYR | 1456 | −12.663 | −14.704 | 65.634 | 1.00 | 23.92 |
| ATOM | 9886 | OH | TYR | 1456 | −11.936 | −13.618 | 65.204 | 1.00 | 24.81 |
| ATOM | 9887 | C | TYR | 1456 | −17.155 | −16.671 | 66.208 | 1.00 | 27.24 |
| ATOM | 9888 | O | TYR | 1456 | −17.610 | −17.308 | 65.259 | 1.00 | 27.22 |
| ATOM | 9889 | N | PRO | 1457 | −17.178 | −15.328 | 66.242 | 1.00 | 28.94 |
| ATOM | 9890 | CD | PRO | 1457 | −17.470 | −14.508 | 65.053 | 1.00 | 28.55 |
| ATOM | 9891 | CA | PRO | 1457 | −16.660 | −14.494 | 67.331 | 1.00 | 29.57 |
| ATOM | 9892 | CB | PRO | 1457 | −16.370 | −13.167 | 66.638 | 1.00 | 29.55 |
| ATOM | 9893 | CG | PRO | 1457 | −17.442 | −13.105 | 65.607 | 1.00 | 31.60 |
| ATOM | 9894 | C | PRO | 1457 | −17.634 | −14.338 | 68.499 | 1.00 | 31.24 |
| ATOM | 9895 | O | PRO | 1457 | −18.850 | −14.454 | 68.333 | 1.00 | 31.53 |
| ATOM | 9896 | N | GLY | 1458 | −17.085 | −14.081 | 69.683 | 1.00 | 32.29 |
| ATOM | 9897 | CA | GLY | 1458 | −17.911 | −13.903 | 70.864 | 1.00 | 33.07 |
| ATOM | 9898 | C | GLY | 1458 | −18.126 | −12.429 | 71.129 | 1.00 | 34.59 |
| ATOM | 9899 | O | GLY | 1458 | −17.564 | −11.583 | 70.431 | 1.00 | 34.41 |
| ATOM | 9900 | N | GLU | 1459 | −18.931 | −12.106 | 72.135 | 1.00 | 34.02 |
| ATOM | 9901 | CA | GLU | 1459 | −19.191 | −10.711 | 72.447 | 1.00 | 35.57 |
| ATOM | 9902 | CB | GLU | 1459 | −20.174 | −10.591 | 73.616 | 1.00 | 39.24 |
| ATOM | 9903 | CG | GLU | 1459 | −21.239 | −9.530 | 73.394 | 1.00 | 41.27 |
| ATOM | 9904 | CD | GLU | 1459 | −22.226 | −9.926 | 72.309 | 1.00 | 42.55 |
| ATOM | 9905 | OE1 | GLU | 1459 | −22.962 | −9.046 | 71.815 | 1.00 | 43.59 |
| ATOM | 9906 | OE2 | GLU | 1459 | −22.272 | −11.123 | 71.954 | 1.00 | 44.59 |
| ATOM | 9907 | C | GLU | 1459 | −17.886 | −10.004 | 72.798 | 1.00 | 34.39 |
| ATOM | 9908 | O | GLU | 1459 | −17.763 | −8.790 | 72.634 | 1.00 | 34.39 |
| ATOM | 9909 | N | GLU | 1460 | −16.910 | −10.775 | 73.271 | 1.00 | 33.94 |
| ATOM | 9910 | CA | GLU | 1460 | −15.607 | −10.232 | 73.645 | 1.00 | 34.54 |
| ATOM | 9911 | CB | GLU | 1460 | −14.784 | −11.277 | 74.404 | 1.00 | 37.12 |
| ATOM | 9912 | CG | GLU | 1460 | −15.592 | −12.160 | 75.331 | 1.00 | 41.64 |
| ATOM | 9913 | CD | GLU | 1460 | −16.379 | −13.213 | 74.579 | 1.00 | 43.05 |
| ATOM | 9914 | OE1 | GLU | 1460 | −15.745 | −14.100 | 73.961 | 1.00 | 43.71 |
| ATOM | 9915 | OE2 | GLU | 1460 | −17.628 | −13.148 | 74.600 | 1.00 | 44.97 |
| ATOM | 9916 | C | GLU | 1460 | −14.828 | −9.816 | 72.404 | 1.00 | 33.32 |
| ATOM | 9917 | O | GLU | 1460 | −13.883 | −9.031 | 72.488 | 1.00 | 33.42 |
| ATOM | 9918 | N | HIS | 1461 | −15.227 | −10.358 | 71.258 | 1.00 | 31.67 |
| ATOM | 9919 | CA | HIS | 1461 | −14.571 | −10.067 | 69.986 | 1.00 | 30.54 |
| ATOM | 9920 | CB | HIS | 1461 | −14.304 | −11.368 | 69.224 | 1.00 | 30.83 |
| ATOM | 9921 | CG | HIS | 1461 | −13.527 | −12.382 | 70.002 | 1.00 | 30.96 |
| ATOM | 9922 | CD2 | HIS | 1461 | −13.853 | −13.630 | 70.415 | 1.00 | 30.87 |
| ATOM | 9923 | ND1 | HIS | 1461 | −12.236 | −12.160 | 70.433 | 1.00 | 31.51 |
| ATOM | 9924 | CE1 | HIS | 1461 | −11.800 | −13.228 | 71.077 | 1.00 | 32.78 |
| ATOM | 9925 | NE2 | HIS | 1461 | −12.761 | −14.134 | 71.080 | 1.00 | 30.95 |
| ATOM | 9926 | C | HIS | 1461 | −15.443 | −9.169 | 69.121 | 1.00 | 30.10 |
| ATOM | 9927 | O | HIS | 1461 | −15.096 | −8.871 | 67.979 | 1.00 | 28.36 |
| ATOM | 9928 | N | SER | 1462 | −16.573 | −8.742 | 69.677 | 1.00 | 29.88 |
| ATOM | 9929 | CA | SER | 1462 | −17.526 | −7.901 | 68.962 | 1.00 | 31.52 |
| ATOM | 9930 | CB | SER | 1462 | −18.930 | −8.484 | 69.111 | 1.00 | 29.18 |
| ATOM | 9931 | OG | SER | 1462 | −18.956 | −9.846 | 68.726 | 1.00 | 31.95 |
| ATOM | 9932 | C | SER | 1462 | −17.538 | −6.453 | 69.440 | 1.00 | 33.19 |
| ATOM | 9933 | O | SER | 1462 | −17.161 | −6.161 | 70.576 | 1.00 | 33.68 |
| ATOM | 9934 | N | PHE | 1463 | −17.981 | −5.550 | 68.567 | 1.00 | 33.32 |
| ATOM | 9935 | CA | PHE | 1463 | −18.057 | −4.130 | 68.900 | 1.00 | 34.66 |
| ATOM | 9936 | CB | PHE | 1463 | −17.364 | −3.274 | 67.834 | 1.00 | 35.23 |
| ATOM | 9937 | CG | PHE | 1463 | −15.895 | −3.555 | 67.684 | 1.00 | 36.25 |
| ATOM | 9938 | CD1 | PHE | 1463 | −15.453 | −4.650 | 66.951 | 1.00 | 36.98 |
| ATOM | 9939 | CD2 | PHE | 1463 | −14.952 | −2.721 | 68.278 | 1.00 | 37.01 |
| ATOM | 9940 | CE1 | PHE | 1463 | −14.092 | −4.910 | 66.807 | 1.00 | 37.76 |
| ATOM | 9941 | CE2 | PHE | 1463 | −13.587 | −2.971 | 68.142 | 1.00 | 35.48 |
| ATOM | 9942 | CZ | PHE | 1463 | −13.157 | −4.068 | 67.405 | 1.00 | 37.03 |
| ATOM | 9943 | C | PHE | 1463 | −19.513 | −3.685 | 69.027 | 1.00 | 35.85 |
| ATOM | 9944 | O | PHE | 1463 | −20.420 | −4.319 | 68.486 | 1.00 | 33.90 |
| ATOM | 9945 | N | HIS | 1464 | −19.727 | −2.585 | 69.740 | 1.00 | 37.39 |
| ATOM | 9946 | CA | HIS | 1464 | −21.068 | −2.053 | 69.949 | 1.00 | 39.80 |
| ATOM | 9947 | CB | HIS | 1464 | −21.623 | −2.538 | 71.289 | 1.00 | 39.63 |
| ATOM | 9948 | CG | HIS | 1464 | −21.853 | −4.016 | 71.342 | 1.00 | 40.65 |
| ATOM | 9949 | CD2 | HIS | 1464 | −21.226 | −4.995 | 72.038 | 1.00 | 40.98 |
| ATOM | 9950 | ND1 | HIS | 1464 | −22.818 | −4.644 | 70.585 | 1.00 | 39.94 |
| ATOM | 9951 | CE1 | HIS | 1464 | −22.776 | −5.945 | 70.810 | 1.00 | 40.36 |
| ATOM | 9952 | NE2 | HIS | 1464 | −21.818 | −6.185 | 71.688 | 1.00 | 40.96 |
| ATOM | 9953 | C | HIS | 1464 | −21.042 | −0.535 | 69.920 | 1.00 | 40.95 |
| ATOM | 9954 | O | HIS | 1464 | −21.911 | 0.059 | 69.243 | 1.00 | 41.98 |
| ATOM | 9955 | OXT | HIS | 1464 | −20.156 | 0.038 | 70.583 | 1.00 | 42.55 |
| ATOM | 9956 | C1 | KPL | 1465 | −8.662 | −8.598 | 56.997 | 1.00 | 40.66 |
| ATOM | 9957 | C2 | KPL | 1465 | −9.406 | −8.731 | 55.651 | 1.00 | 40.02 |
| ATOM | 9958 | C3 | KPL | 1465 | −9.470 | −10.212 | 55.264 | 1.00 | 40.28 |
| ATOM | 9959 | C4 | KPL | 1465 | −10.847 | −8.205 | 55.810 | 1.00 | 41.40 |
| ATOM | 9960 | O1 | KPL | 1465 | −10.844 | −6.822 | 56.181 | 1.00 | 43.78 |
| ATOM | 9961 | C5 | KPL | 1465 | −8.646 | −7.947 | 54.549 | 1.00 | 38.37 |
| ATOM | 9962 | O2 | KPL | 1465 | −9.203 | −7.052 | 53.945 | 1.00 | 39.08 |
| ATOM | 9963 | C6 | KPL | 1465 | −7.208 | −8.268 | 54.193 | 1.00 | 36.59 |
| ATOM | 9964 | O3 | KPL | 1465 | −6.611 | −9.157 | 54.768 | 1.00 | 34.92 |
| ATOM | 9965 | O4 | KPL | 1465 | −6.578 | −7.561 | 53.231 | 1.00 | 31.35 |
| ATOM | 9966 | CB | MET | 1501 | 12.451 | −25.585 | −6.577 | 1.00 | 68.45 |
| ATOM | 9967 | CG | MET | 1501 | 12.983 | −25.086 | −7.922 | 1.00 | 70.78 |
| ATOM | 9968 | SD | MET | 1501 | 14.555 | −25.814 | −8.446 | 1.00 | 73.21 |
| ATOM | 9969 | CE | MET | 1501 | 15.640 | −24.379 | −8.381 | 1.00 | 72.59 |
| ATOM | 9970 | C | MET | 1501 | 14.232 | −26.660 | −5.175 | 1.00 | 64.43 |
| ATOM | 9971 | O | MET | 1501 | 13.933 | −27.714 | −5.740 | 1.00 | 64.33 |
| ATOM | 9972 | N | MET | 1501 | 14.252 | −24.188 | −5.606 | 1.00 | 67.20 |
| ATOM | 9973 | CA | MET | 1501 | 13.404 | −25.395 | −5.390 | 1.00 | 66.44 |
| ATOM | 9974 | N | LYS | 1502 | 15.270 | −26.551 | −4.351 | 1.00 | 61.82 |
| ATOM | 9975 | CA | LYS | 1502 | 16.137 | −27.690 | −4.067 | 1.00 | 58.51 |
| ATOM | 9976 | CB | LYS | 1502 | 17.506 | −27.493 | −4.719 | 1.00 | 59.36 |
| ATOM | 9977 | CG | LYS | 1502 | 17.454 | −27.205 | −6.219 | 1.00 | 59.84 |
| ATOM | 9978 | CD | LYS | 1502 | 16.747 | −28.314 | −6.992 | 1.00 | 60.21 |
| ATOM | 9979 | CE | LYS | 1502 | 17.479 | −29.641 | −6.871 | 1.00 | 60.21 |
| ATOM | 9980 | NZ | LYS | 1502 | 16.766 | −30.731 | −7.593 | 1.00 | 60.90 |
| ATOM | 9981 | C | LYS | 1502 | 16.286 | −27.938 | −2.562 | 1.00 | 55.49 |
| ATOM | 9982 | O | LYS | 1502 | 16.023 | −29.039 | −2.083 | 1.00 | 56.98 |
| ATOM | 9983 | N | PRO | 1503 | 16.733 | −26.918 | −1.795 | 1.00 | 51.21 |
| ATOM | 9984 | CD | PRO | 1503 | 16.674 | −27.002 | −0.323 | 1.00 | 49.69 |
| ATOM | 9985 | CA | PRO | 1503 | 17.082 | −25.554 | −2.213 | 1.00 | 46.71 |
| ATOM | 9986 | CB | PRO | 1503 | 16.764 | −24.739 | −0.973 | 1.00 | 48.28 |
| ATOM | 9987 | CG | PRO | 1503 | 17.226 | −25.653 | 0.112 | 1.00 | 49.41 |
| ATOM | 9988 | C | PRO | 1503 | 18.558 | −25.434 | −2.610 | 1.00 | 42.37 |
| ATOM | 9989 | O | PRO | 1503 | 19.337 | −26.369 | −2.433 | 1.00 | 41.84 |
| ATOM | 9990 | N | THR | 1504 | 18.929 | −24.274 | −3.142 | 1.00 | 37.50 |
| ATOM | 9991 | CA | THR | 1504 | 20.302 | −24.024 | −3.565 | 1.00 | 33.10 |
| ATOM | 9992 | CB | THR | 1504 | 20.375 | −22.784 | −4.485 | 1.00 | 32.53 |
| ATOM | 9993 | OG1 | THR | 1504 | 19.578 | −23.011 | −5.653 | 1.00 | 33.15 |
| ATOM | 9994 | CG2 | THR | 1504 | 21.808 | −22.502 | −4.905 | 1.00 | 29.59 |
| ATOM | 9995 | C | THR | 1504 | 21.190 | −23.805 | −2.342 | 1.00 | 31.99 |
| ATOM | 9996 | O | THR | 1504 | 20.826 | −23.069 | −1.425 | 1.00 | 29.30 |
| ATOM | 9997 | N | THR | 1505 | 22.354 | −24.446 | −2.332 | 1.00 | 30.78 |
| ATOM | 9998 | CA | THR | 1505 | 23.273 | −24.324 | −1.208 | 1.00 | 29.63 |
| ATOM | 9999 | CB | THR | 1505 | 23.305 | −25.619 | −0.369 | 1.00 | 29.85 |
| ATOM | 10000 | OG1 | THR | 1505 | 23.894 | −26.674 | −1.138 | 1.00 | 31.33 |
| ATOM | 10001 | CG2 | THR | 1505 | 21.899 | −26.029 | 0.036 | 1.00 | 29.10 |
| ATOM | 10002 | C | THR | 1505 | 24.695 | −24.014 | −1.659 | 1.00 | 29.48 |
| ATOM | 10003 | O | THR | 1505 | 25.006 | −24.056 | −2.850 | 1.00 | 27.82 |
| ATOM | 10004 | N | ILE | 1506 | 25.557 | −23.708 | −0.693 | 1.00 | 29.78 |
| ATOM | 10005 | CA | ILE | 1506 | 26.950 | −23.391 | −0.975 | 1.00 | 30.15 |
| ATOM | 10006 | CB | ILE | 1506 | 27.723 | −23.108 | 0.331 | 1.00 | 31.02 |
| ATOM | 10007 | CG2 | ILE | 1506 | 29.122 | −22.597 | 0.013 | 1.00 | 30.67 |
| ATOM | 10008 | CG1 | ILE | 1506 | 26.966 | −22.065 | 1.161 | 1.00 | 31.55 |
| ATOM | 10009 | CD1 | ILE | 1506 | 27.492 | −21.896 | 2.576 | 1.00 | 33.42 |
| ATOM | 10010 | C | ILE | 1506 | 27.593 | −24.568 | −1.705 | 1.00 | 30.17 |
| ATOM | 10011 | O | ILE | 1506 | 28.525 | −24.394 | −2.491 | 1.00 | 30.44 |
| ATOM | 10012 | N | SER | 1507 | 27.077 | −25.765 | −1.441 | 1.00 | 31.25 |
| ATOM | 10013 | CA | SER | 1507 | 27.576 | −26.990 | −2.059 | 1.00 | 33.43 |
| ATOM | 10014 | CB | SER | 1507 | 26.707 | −28.180 | −1.646 | 1.00 | 34.53 |
| ATOM | 10015 | OG | SER | 1507 | 26.578 | −28.258 | −0.238 | 1.00 | 39.65 |
| ATOM | 10016 | C | SER | 1507 | 27.579 | −26.889 | −3.580 | 1.00 | 33.68 |
| ATOM | 10017 | O | SER | 1507 | 28.551 | −27.269 | −4.234 | 1.00 | 33.38 |
| ATOM | 10018 | N | LEU | 1508 | 26.483 | −26.383 | −4.137 | 1.00 | 32.89 |
| ATOM | 10019 | CA | LEU | 1508 | 26.361 | −26.242 | −5.582 | 1.00 | 32.80 |
| ATOM | 10020 | CB | LEU | 1508 | 24.970 | −25.720 | −5.958 | 1.00 | 34.32 |
| ATOM | 10021 | CG | LEU | 1508 | 24.031 | −26.678 | −6.701 | 1.00 | 36.26 |
| ATOM | 10022 | CD1 | LEU | 1508 | 22.694 | −25.992 | −6.951 | 1.00 | 36.00 |
| ATOM | 10023 | CD2 | LEU | 1508 | 24.661 | −27.114 | −8.009 | 1.00 | 36.33 |
| ATOM | 10024 | C | LEU | 1508 | 27.420 | −25.307 | −6.156 | 1.00 | 31.90 |
| ATOM | 10025 | O | LEU | 1508 | 28.012 | −25.597 | −7.196 | 1.00 | 32.06 |
| ATOM | 10026 | N | LEU | 1509 | 27.659 | −24.187 | −5.482 | 1.00 | 29.72 |
| ATOM | 10027 | CA | LEU | 1509 | 28.646 | −23.226 | −5.959 | 1.00 | 29.54 |
| ATOM | 10028 | CB | LEU | 1509 | 28.595 | −21.946 | −5.116 | 1.00 | 29.54 |
| ATOM | 10029 | CG | LEU | 1509 | 27.233 | −21.237 | −5.105 | 1.00 | 29.15 |
| ATOM | 10030 | CD1 | LEU | 1509 | 27.350 | −19.898 | −4.383 | 1.00 | 30.33 |
| ATOM | 10031 | CD2 | LEU | 1509 | 26.748 | −21.021 | −6.535 | 1.00 | 28.87 |
| ATOM | 10032 | C | LEU | 1509 | 30.044 | −23.830 | −5.927 | 1.00 | 30.18 |
| ATOM | 10033 | O | LEU | 1509 | 30.875 | −23.559 | −6.799 | 1.00 | 27.72 |
| ATOM | 10034 | N | GLN | 1510 | 30.294 | −24.658 | −4.920 | 1.00 | 30.60 |
| ATOM | 10035 | CA | GLN | 1510 | 31.587 | −25.308 | −4.779 | 1.00 | 32.89 |
| ATOM | 10036 | CB | GLN | 1510 | 31.674 | −26.008 | −3.419 | 1.00 | 32.23 |
| ATOM | 10037 | CG | GLN | 1510 | 33.059 | −26.521 | −3.051 | 1.00 | 34.03 |
| ATOM | 10038 | CD | GLN | 1510 | 34.137 | −25.452 | −3.146 | 1.00 | 35.52 |
| ATOM | 10039 | OE1 | GLN | 1510 | 34.673 | −25.187 | −4.223 | 1.00 | 36.22 |
| ATOM | 10040 | NE2 | GLN | 1510 | 34.452 | −24.824 | −2.017 | 1.00 | 34.94 |
| ATOM | 10041 | C | GLN | 1510 | 31.747 | −26.312 | −5.920 | 1.00 | 33.79 |
| ATOM | 10042 | O | GLN | 1510 | 32.852 | −26.537 | −6.408 | 1.00 | 34.78 |
| ATOM | 10043 | N | LYS | 1511 | 30.636 | −26.906 | −6.345 | 1.00 | 35.14 |
| ATOM | 10044 | CA | LYS | 1511 | 30.659 | −27.864 | −7.446 | 1.00 | 36.98 |
| ATOM | 10045 | CB | LYS | 1511 | 29.309 | −28.574 | −7.574 | 1.00 | 38.13 |
| ATOM | 10046 | CG | LYS | 1511 | 29.169 | −29.386 | −8.859 | 1.00 | 40.56 |
| ATOM | 10047 | CD | LYS | 1511 | 27.711 | −29.594 | −9.259 | 1.00 | 43.87 |
| ATOM | 10048 | CE | LYS | 1511 | 26.973 | −30.517 | −8.299 | 1.00 | 45.00 |
| ATOM | 10049 | NZ | LYS | 1511 | 25.536 | −30.670 | −8.686 | 1.00 | 47.90 |
| ATOM | 10050 | C | LYS | 1511 | 30.955 | −27.127 | −8.750 | 1.00 | 36.69 |
| ATOM | 10051 | O | LYS | 1511 | 31.694 | −27.619 | −9.599 | 1.00 | 37.22 |
| ATOM | 10052 | N | TYR | 1512 | 30.371 | −25.942 | −8.899 | 1.00 | 36.54 |
| ATOM | 10053 | CA | TYR | 1512 | 30.560 | −25.141 | −10.098 | 1.00 | 35.65 |
| ATOM | 10054 | CB | TYR | 1512 | 29.669 | −23.900 | −10.053 | 1.00 | 37.28 |
| ATOM | 10055 | CG | TYR | 1512 | 28.193 | −24.188 | −10.212 | 1.00 | 39.20 |
| ATOM | 10056 | CD1 | TYR | 1512 | 27.249 | −23.175 | −10.051 | 1.00 | 41.06 |
| ATOM | 10057 | CE1 | TYR | 1512 | 25.889 | −23.429 | −10.195 | 1.00 | 42.03 |
| ATOM | 10058 | CD2 | TYR | 1512 | 27.739 | −25.468 | −10.526 | 1.00 | 40.61 |
| ATOM | 10059 | CE2 | TYR | 1512 | 26.382 | −25.734 | −10.673 | 1.00 | 41.29 |
| ATOM | 10060 | CZ | TYR | 1512 | 25.463 | −24.710 | −10.506 | 1.00 | 42.07 |
| ATOM | 10061 | OH | TYR | 1512 | 24.117 | −24.963 | −10.646 | 1.00 | 43.50 |
| ATOM | 10062 | C | TYR | 1512 | 32.002 | −24.716 | −10.328 | 1.00 | 36.33 |
| ATOM | 10063 | O | TYR | 1512 | 32.492 | −24.771 | −11.457 | 1.00 | 34.44 |
| ATOM | 10064 | N | LYS | 1513 | 32.684 | −24.277 | −9.274 | 1.00 | 35.91 |
| ATOM | 10065 | CA | LYS | 1513 | 34.074 | −23.858 | −9.426 | 1.00 | 37.24 |
| ATOM | 10066 | CB | LYS | 1513 | 34.646 | −23.362 | −8.090 | 1.00 | 35.07 |
| ATOM | 10067 | CG | LYS | 1513 | 36.146 | −23.108 | −8.136 | 1.00 | 32.35 |
| ATOM | 10068 | CD | LYS | 1513 | 36.594 | −22.091 | −7.096 | 1.00 | 31.84 |
| ATOM | 10069 | CE | LYS | 1513 | 38.093 | −21.848 | −7.198 | 1.00 | 30.35 |
| ATOM | 10070 | NZ | LYS | 1513 | 38.524 | −20.591 | −6.529 | 1.00 | 29.60 |
| ATOM | 10071 | C | LYS | 1513 | 34.907 | −25.022 | −9.954 | 1.00 | 38.69 |
| ATOM | 10072 | O | LYS | 1513 | 35.870 | −24.829 | −10.694 | 1.00 | 39.24 |
| ATOM | 10073 | N | GLN | 1514 | 34.517 | −26.231 | −9.567 | 1.00 | 40.67 |
| ATOM | 10074 | CA | GLN | 1514 | 35.205 | −27.437 | −9.995 | 1.00 | 43.38 |
| ATOM | 10075 | CB | GLN | 1514 | 34.733 | −28.623 | −9.160 | 1.00 | 45.15 |
| ATOM | 10076 | CG | GLN | 1514 | 35.177 | −28.565 | −7.710 | 1.00 | 48.35 |
| ATOM | 10077 | CD | GLN | 1514 | 34.481 | −29.604 | −6.855 | 1.00 | 49.83 |
| ATOM | 10078 | OE1 | GLN | 1514 | 34.101 | −30.669 | −7.343 | 1.00 | 51.33 |
| ATOM | 10079 | NE2 | GLN | 1514 | 34.321 | −29.304 | −5.570 | 1.00 | 50.47 |
| ATOM | 10080 | C | GLN | 1514 | 34.936 | −27.704 | −11.470 | 1.00 | 43.74 |
| ATOM | 10081 | O | GLN | 1514 | 35.820 | −28.148 | −12.200 | 1.00 | 44.54 |
| ATOM | 10082 | N | GLU | 1515 | 33.710 | −27.428 | −11.902 | 1.00 | 43.52 |
| ATOM | 10083 | CA | GLU | 1515 | 33.329 | −27.637 | −13.291 | 1.00 | 43.19 |
| ATOM | 10084 | CB | GLU | 1515 | 31.830 | −27.920 | −13.394 | 1.00 | 44.57 |
| ATOM | 10085 | CG | GLU | 1515 | 31.351 | −29.051 | −12.502 | 1.00 | 46.62 |
| ATOM | 10086 | CD | GLU | 1515 | 29.909 | −29.430 | −12.773 | 1.00 | 48.59 |
| ATOM | 10087 | OE1 | GLU | 1515 | 29.051 | −28.525 | −12.825 | 1.00 | 48.92 |
| ATOM | 10088 | OE2 | GLU | 1515 | 29.630 | −30.637 | −12.927 | 1.00 | 51.45 |
| ATOM | 10089 | C | GLU | 1515 | 33.676 | −26.417 | −14.136 | 1.00 | 42.44 |
| ATOM | 10090 | O | GLU | 1515 | 33.426 | −26.396 | −15.343 | 1.00 | 43.10 |
| ATOM | 10091 | N | LYS | 1516 | 34.255 | −25.405 | −13.496 | 1.00 | 40.37 |
| ATOM | 10092 | CA | LYS | 1516 | 34.633 | −24.169 | −14.175 | 1.00 | 39.50 |
| ATOM | 10093 | CB | LYS | 1516 | 35.645 | −24.459 | −15.282 | 1.00 | 40.90 |
| ATOM | 10094 | CG | LYS | 1516 | 36.965 | −25.031 | −14.793 | 1.00 | 42.75 |
| ATOM | 10095 | CD | LYS | 1516 | 37.714 | −24.039 | −13.919 | 1.00 | 45.19 |
| ATOM | 10096 | CE | LYS | 1516 | 39.074 | −24.585 | −13.498 | 1.00 | 46.49 |
| ATOM | 10097 | NZ | LYS | 1516 | 39.950 | −24.871 | −14.670 | 1.00 | 48.49 |
| ATOM | 10098 | C | LYS | 1516 | 33.408 | −23.475 | −14.768 | 1.00 | 38.14 |
| ATOM | 10099 | O | LYS | 1516 | 33.512 | −22.731 | −15.745 | 1.00 | 38.51 |
| ATOM | 10100 | N | LYS | 1517 | 32.249 | −23.727 | −14.170 | 1.00 | 35.26 |
| ATOM | 10101 | CA | LYS | 1517 | 31.003 | −23.128 | −14.630 | 1.00 | 33.69 |
| ATOM | 10102 | CB | LYS | 1517 | 29.837 | −24.084 | −14.385 | 1.00 | 34.32 |
| ATOM | 10103 | CG | LYS | 1517 | 28.497 | −23.587 | −14.904 | 1.00 | 35.09 |
| ATOM | 10104 | CD | LYS | 1517 | 27.352 | −24.446 | −14.384 | 1.00 | 37.44 |
| ATOM | 10105 | CE | LYS | 1517 | 27.483 | −25.896 | −14.832 | 1.00 | 39.19 |
| ATOM | 10106 | NZ | LYS | 1517 | 26.503 | −26.777 | −14.131 | 1.00 | 41.39 |
| ATOM | 10107 | C | LYS | 1517 | 30.765 | −21.823 | −13.878 | 1.00 | 31.87 |
| ATOM | 10108 | O | LYS | 1517 | 30.366 | −21.830 | −12.715 | 1.00 | 31.03 |
| ATOM | 10109 | N | ARG | 1518 | 31.018 | −20.704 | −14.546 | 1.00 | 30.53 |
| ATOM | 10110 | CA | ARG | 1518 | 30.826 | −19.399 | −13.929 | 1.00 | 28.43 |
| ATOM | 10111 | CB | ARG | 1518 | 31.462 | −18.319 | −14.805 | 1.00 | 30.06 |
| ATOM | 10112 | CG | ARG | 1518 | 32.981 | −18.306 | −14.714 | 1.00 | 31.46 |
| ATOM | 10113 | CD | ARG | 1518 | 33.626 | −17.421 | −15.766 | 1.00 | 33.23 |
| ATOM | 10114 | NE | ARG | 1518 | 33.666 | −18.071 | −17.074 | 1.00 | 35.53 |
| ATOM | 10115 | CZ | ARG | 1518 | 34.192 | −17.523 | −18.165 | 1.00 | 36.48 |
| ATOM | 10116 | NH1 | ARG | 1518 | 34.723 | −16.308 | −18.113 | 1.00 | 34.15 |
| ATOM | 10117 | NH2 | ARG | 1518 | 34.192 | −18.194 | −19.308 | 1.00 | 37.73 |
| ATOM | 10118 | C | ARG | 1518 | 29.340 | −19.127 | −13.692 | 1.00 | 27.86 |
| ATOM | 10119 | O | ARG | 1518 | 28.518 | −19.234 | −14.605 | 1.00 | 27.30 |
| ATOM | 10120 | N | PHE | 1519 | 29.008 | −18.776 | −12.452 | 1.00 | 25.52 |
| ATOM | 10121 | CA | PHE | 1519 | 27.625 | −18.518 | −12.056 | 1.00 | 23.05 |
| ATOM | 10122 | CB | PHE | 1519 | 27.283 | −19.382 | −10.841 | 1.00 | 23.24 |
| ATOM | 10123 | CG | PHE | 1519 | 28.151 | −19.109 | −9.646 | 1.00 | 22.05 |
| ATOM | 10124 | CD1 | PHE | 1519 | 27.829 | −18.085 | −8.755 | 1.00 | 22.20 |
| ATOM | 10125 | CD2 | PHE | 1519 | 29.311 | −19.851 | −9.428 | 1.00 | 24.57 |
| ATOM | 10126 | CE1 | PHE | 1519 | 28.650 | −17.800 | −7.666 | 1.00 | 20.88 |
| ATOM | 10127 | CE2 | PHE | 1519 | 30.142 | −19.575 | −8.342 | 1.00 | 24.45 |
| ATOM | 10128 | CZ | PHE | 1519 | 29.810 | −18.547 | −7.457 | 1.00 | 23.26 |
| ATOM | 10129 | C | PHE | 1519 | 27.356 | −17.048 | −11.738 | 1.00 | 23.05 |
| ATOM | 10130 | O | PHE | 1519 | 28.248 | −16.314 | −11.310 | 1.00 | 21.97 |
| ATOM | 10131 | N | ALA | 1520 | 26.113 | −16.627 | −11.944 | 1.00 | 21.46 |
| ATOM | 10132 | CA | ALA | 1520 | 25.718 | −15.250 | −11.687 | 1.00 | 20.80 |
| ATOM | 10133 | CB | ALA | 1520 | 24.835 | −14.747 | −12.822 | 1.00 | 20.77 |
| ATOM | 10134 | C | ALA | 1520 | 24.991 | −15.098 | −10.352 | 1.00 | 19.62 |
| ATOM | 10135 | O | ALA | 1520 | 24.241 | −15.980 | −9.936 | 1.00 | 20.43 |
| ATOM | 10136 | N | THR | 1521 | 25.230 | −13.972 | −9.688 | 1.00 | 19.21 |
| ATOM | 10137 | CA | THR | 1521 | 24.602 | −13.663 | −8.406 | 1.00 | 20.28 |
| ATOM | 10138 | CB | THR | 1521 | 25.626 | −13.736 | −7.259 | 1.00 | 20.78 |
| ATOM | 10139 | OG1 | THR | 1521 | 26.154 | −15.070 | −7.183 | 1.00 | 23.74 |
| ATOM | 10140 | CG2 | THR | 1521 | 24.967 | −13.386 | −5.934 | 1.00 | 27.16 |
| ATOM | 10141 | C | THR | 1521 | 24.030 | −12.250 | −8.495 | 1.00 | 17.98 |
| ATOM | 10142 | O | THR | 1521 | 24.485 | −11.435 | −9.300 | 1.00 | 17.51 |
| ATOM | 10143 | N | ILE | 1522 | 23.042 | −11.942 | −7.666 | 1.00 | 17.55 |
| ATOM | 10144 | CA | ILE | 1522 | 22.427 | −10.626 | −7.746 | 1.00 | 15.48 |
| ATOM | 10145 | CB | ILE | 1522 | 21.232 | −10.684 | −8.726 | 1.00 | 15.93 |
| ATOM | 10146 | CG2 | ILE | 1522 | 20.049 | −11.381 | −8.064 | 1.00 | 15.09 |
| ATOM | 10147 | CG1 | ILE | 1522 | 20.846 | −9.276 | −9.185 | 1.00 | 15.40 |
| ATOM | 10148 | CD1 | ILE | 1522 | 21.865 | −8.630 | −10.104 | 1.00 | 21.34 |
| ATOM | 10149 | C | ILE | 1522 | 21.944 | −10.131 | −6.386 | 1.00 | 15.61 |
| ATOM | 10150 | O | ILE | 1522 | 21.694 | −10.926 | −5.485 | 1.00 | 13.97 |
| ATOM | 10151 | N | THR | 1523 | 21.825 | −8.818 | −6.226 | 1.00 | 17.24 |
| ATOM | 10152 | CA | THR | 1523 | 21.331 | −8.289 | −4.962 | 1.00 | 16.39 |
| ATOM | 10153 | CB | THR | 1523 | 21.855 | −6.859 | −4.657 | 1.00 | 16.96 |
| ATOM | 10154 | OG1 | THR | 1523 | 21.353 | −5.938 | −5.628 | 1.00 | 18.49 |
| ATOM | 10155 | CG2 | THR | 1523 | 23.385 | −6.828 | −4.660 | 1.00 | 17.93 |
| ATOM | 10156 | C | THR | 1523 | 19.806 | −8.255 | −5.065 | 1.00 | 15.12 |
| ATOM | 10157 | O | THR | 1523 | 19.250 | −8.196 | −6.156 | 1.00 | 15.19 |
| ATOM | 10158 | N | ALA | 1524 | 19.131 | −8.323 | −3.925 | 1.00 | 15.23 |
| ATOM | 10159 | CA | ALA | 1524 | 17.675 | −8.280 | −3.889 | 1.00 | 12.50 |
| ATOM | 10160 | CB | ALA | 1524 | 17.092 | −9.670 | −4.121 | 1.00 | 13.98 |
| ATOM | 10161 | C | ALA | 1524 | 17.296 | −7.759 | −2.508 | 1.00 | 13.20 |
| ATOM | 10162 | O | ALA | 1524 | 17.943 | −8.102 | −1.517 | 1.00 | 14.00 |
| ATOM | 10163 | N | TYR | 1525 | 16.244 | −6.946 | −2.448 | 1.00 | 13.12 |
| ATOM | 10164 | CA | TYR | 1525 | 15.813 | −6.346 | −1.190 | 1.00 | 13.36 |
| ATOM | 10165 | CB | TYR | 1525 | 16.287 | −4.896 | −1.112 | 1.00 | 12.75 |
| ATOM | 10166 | CG | TYR | 1525 | 17.633 | −4.646 | −1.748 | 1.00 | 14.96 |
| ATOM | 10167 | CD1 | TYR | 1525 | 17.717 | −4.174 | −3.056 | 1.00 | 16.08 |
| ATOM | 10168 | CE1 | TYR | 1525 | 18.938 | −3.904 | −3.648 | 1.00 | 18.05 |
| ATOM | 10169 | CD2 | TYR | 1525 | 18.820 | −4.852 | −1.040 | 1.00 | 15.12 |
| ATOM | 10170 | CE2 | TYR | 1525 | 20.064 | −4.582 | −1.627 | 1.00 | 14.69 |
| ATOM | 10171 | CZ | TYR | 1525 | 20.107 | −4.107 | −2.928 | 1.00 | 16.54 |
| ATOM | 10172 | OH | TYR | 1525 | 21.315 | −3.807 | −3.521 | 1.00 | 17.02 |
| ATOM | 10173 | C | TYR | 1525 | 14.305 | −6.357 | −0.977 | 1.00 | 13.91 |
| ATOM | 10174 | O | TYR | 1525 | 13.808 | −5.714 | −0.053 | 1.00 | 12.93 |
| ATOM | 10175 | N | ASP | 1526 | 13.575 | −7.055 | −1.839 | 1.00 | 12.13 |
| ATOM | 10176 | CA | ASP | 1526 | 12.129 | −7.127 | −1.692 | 1.00 | 12.96 |
| ATOM | 10177 | CB | ASP | 1526 | 11.454 | −5.901 | −2.325 | 1.00 | 13.73 |
| ATOM | 10178 | CG | ASP | 1526 | 11.615 | −5.846 | −3.835 | 1.00 | 17.18 |
| ATOM | 10179 | OD1 | ASP | 1526 | 10.998 | −6.672 | −4.543 | 1.00 | 19.08 |
| ATOM | 10180 | OD2 | ASP | 1526 | 12.363 | −4.967 | −4.311 | 1.00 | 19.29 |
| ATOM | 10181 | C | ASP | 1526 | 11.574 | −8.412 | −2.297 | 1.00 | 14.23 |
| ATOM | 10182 | O | ASP | 1526 | 12.250 | −9.088 | −3.071 | 1.00 | 13.08 |
| ATOM | 10183 | N | TYR | 1527 | 10.343 | −8.741 | −1.918 | 1.00 | 11.93 |
| ATOM | 10184 | CA | TYR | 1527 | 9.648 | −9.941 | −2.387 | 1.00 | 13.16 |
| ATOM | 10185 | CB | TYR | 1527 | 8.248 | −9.987 | −1.778 | 1.00 | 14.17 |
| ATOM | 10186 | CG | TYR | 1527 | 7.338 | −11.037 | −2.388 | 1.00 | 16.24 |
| ATOM | 10187 | CD1 | TYR | 1527 | 7.352 | −12.348 | −1.925 | 1.00 | 16.32 |
| ATOM | 10188 | CE1 | TYR | 1527 | 6.527 | −13.320 | −2.484 | 1.00 | 17.69 |
| ATOM | 10189 | CD2 | TYR | 1527 | 6.468 | −10.718 | −3.437 | 1.00 | 17.45 |
| ATOM | 10190 | CE2 | TYR | 1527 | 5.637 | −11.688 | −4.007 | 1.00 | 17.34 |
| ATOM | 10191 | CZ | TYR | 1527 | 5.677 | −12.987 | −3.519 | 1.00 | 19.46 |
| ATOM | 10192 | OH | TYR | 1527 | 4.875 | −13.963 | −4.069 | 1.00 | 21.07 |
| ATOM | 10193 | C | TYR | 1527 | 9.503 | −10.052 | −3.901 | 1.00 | 14.65 |
| ATOM | 10194 | O | TYR | 1527 | 9.766 | −11.102 | −4.480 | 1.00 | 13.71 |
| ATOM | 10195 | N | SER | 1528 | 9.057 | −8.974 | −4.534 | 1.00 | 14.41 |
| ATOM | 10196 | CA | SER | 1528 | 8.825 | −9.001 | −5.965 | 1.00 | 12.84 |
| ATOM | 10197 | CB | SER | 1528 | 8.203 | −7.687 | −6.422 | 1.00 | 13.95 |
| ATOM | 10198 | OG | SER | 1528 | 6.922 | −7.521 | −5.821 | 1.00 | 16.83 |
| ATOM | 10199 | C | SER | 1528 | 10.041 | −9.324 | −6.809 | 1.00 | 13.31 |
| ATOM | 10200 | O | SER | 1528 | 9.991 | −10.229 | −7.650 | 1.00 | 13.87 |
| ATOM | 10201 | N | PHE | 1529 | 11.139 | −8.609 | −6.610 | 1.00 | 13.52 |
| ATOM | 10202 | CA | PHE | 1529 | 12.315 | −8.926 | −7.410 | 1.00 | 14.35 |
| ATOM | 10203 | CB | PHE | 1529 | 13.359 | −7.817 | −7.299 | 1.00 | 12.92 |
| ATOM | 10204 | CG | PHE | 1529 | 13.092 | −6.664 | −8.215 | 1.00 | 16.39 |
| ATOM | 10205 | CD1 | PHE | 1529 | 12.474 | −5.511 | −7.747 | 1.00 | 15.02 |
| ATOM | 10206 | CD2 | PHE | 1529 | 13.443 | −6.743 | −9.560 | 1.00 | 15.32 |
| ATOM | 10207 | CE1 | PHE | 1529 | 12.207 | −4.440 | −8.609 | 1.00 | 17.64 |
| ATOM | 10208 | CE2 | PHE | 1529 | 13.183 | −5.685 | −10.432 | 1.00 | 16.87 |
| ATOM | 10209 | CZ | PHE | 1529 | 12.564 | −4.529 | −9.959 | 1.00 | 17.73 |
| ATOM | 10210 | C | PHE | 1529 | 12.909 | −10.279 | −7.038 | 1.00 | 13.50 |
| ATOM | 10211 | O | PHE | 1529 | 13.384 | −11.010 | −7.908 | 1.00 | 14.17 |
| ATOM | 10212 | N | ALA | 1530 | 12.865 | −10.628 | −5.757 | 1.00 | 13.05 |
| ATOM | 10213 | CA | ALA | 1530 | 13.395 | −11.925 | −5.337 | 1.00 | 13.28 |
| ATOM | 10214 | CB | ALA | 1530 | 13.261 | −12.083 | −3.819 | 1.00 | 11.40 |
| ATOM | 10215 | C | ALA | 1530 | 12.640 | −13.050 | −6.050 | 1.00 | 13.09 |
| ATOM | 10216 | O | ALA | 1530 | 13.242 | −14.003 | −6.546 | 1.00 | 14.85 |
| ATOM | 10217 | N | LYS | 1531 | 11.315 | −12.931 | −6.087 | 1.00 | 13.26 |
| ATOM | 10218 | CA | LYS | 1531 | 10.458 | −13.928 | −6.728 | 1.00 | 14.54 |
| ATOM | 10219 | CB | LYS | 1531 | 8.991 | −13.535 | −6.523 | 1.00 | 15.21 |
| ATOM | 10220 | CG | LYS | 1531 | 7.959 | −14.425 | −7.195 | 1.00 | 19.57 |
| ATOM | 10221 | CD | LYS | 1531 | 7.576 | −15.623 | −6.363 | 1.00 | 24.21 |
| ATOM | 10222 | CE | LYS | 1531 | 6.196 | −16.116 | −6.781 | 1.00 | 25.02 |
| ATOM | 10223 | NZ | LYS | 1531 | 6.169 | −16.393 | −8.238 | 1.00 | 27.58 |
| ATOM | 10224 | C | LYS | 1531 | 10.773 | −14.020 | −8.221 | 1.00 | 13.65 |
| ATOM | 10225 | O | LYS | 1531 | 10.854 | −15.114 | −8.783 | 1.00 | 13.38 |
| ATOM | 10226 | N | LEU | 1532 | 10.941 | −12.863 | −8.853 | 1.00 | 15.51 |
| ATOM | 10227 | CA | LEU | 1532 | 11.238 | −12.793 | −10.283 | 1.00 | 15.37 |
| ATOM | 10228 | CB | LEU | 1532 | 11.295 | −11.326 | −10.727 | 1.00 | 16.67 |
| ATOM | 10229 | CG | LEU | 1532 | 11.485 | −11.051 | −12.233 | 1.00 | 13.94 |
| ATOM | 10230 | CD1 | LEU | 1532 | 11.109 | −9.614 | −12.552 | 1.00 | 15.17 |
| ATOM | 10231 | CD2 | LEU | 1532 | 12.937 | −11.316 | −12.617 | 1.00 | 18.81 |
| ATOM | 10232 | C | LEU | 1532 | 12.555 | −13.507 | −10.605 | 1.00 | 15.94 |
| ATOM | 10233 | O | LEU | 1532 | 12.612 | −14.332 | −11.522 | 1.00 | 18.14 |
| ATOM | 10234 | N | PHE | 1533 | 13.604 | −13.201 | −9.844 | 1.00 | 16.77 |
| ATOM | 10235 | CA | PHE | 1533 | 14.905 | −13.825 | −10.063 | 1.00 | 16.92 |
| ATOM | 10236 | CB | PHE | 1533 | 15.965 | −13.248 | −9.106 | 1.00 | 16.06 |
| ATOM | 10237 | CG | PHE | 1533 | 16.169 | −11.762 | −9.227 | 1.00 | 16.65 |
| ATOM | 10238 | CD1 | PHE | 1533 | 15.971 | −11.111 | −10.440 | 1.00 | 18.16 |
| ATOM | 10239 | CD2 | PHE | 1533 | 16.564 | −11.011 | −8.119 | 1.00 | 14.45 |
| ATOM | 10240 | CE1 | PHE | 1533 | 16.157 | −9.735 | −10.554 | 1.00 | 18.81 |
| ATOM | 10241 | CE2 | PHE | 1533 | 16.754 | −9.632 | −8.225 | 1.00 | 16.25 |
| ATOM | 10242 | CZ | PHE | 1533 | 16.549 | −8.991 | −9.443 | 1.00 | 18.42 |
| ATOM | 10243 | C | PHE | 1533 | 14.814 | −15.337 | −9.858 | 1.00 | 17.37 |
| ATOM | 10244 | O | PHE | 1533 | 15.257 | −16.112 | −10.699 | 1.00 | 17.81 |
| ATOM | 10245 | N | ALA | 1534 | 14.237 | −15.743 | −8.732 | 1.00 | 17.43 |
| ATOM | 10246 | CA | ALA | 1534 | 14.085 | −17.159 | −8.417 | 1.00 | 19.85 |
| ATOM | 10247 | CB | ALA | 1534 | 13.370 | −17.333 | −7.074 | 1.00 | 20.47 |
| ATOM | 10248 | C | ALA | 1534 | 13.316 | −17.900 | −9.509 | 1.00 | 21.04 |
| ATOM | 10249 | O | ALA | 1534 | 13.616 | −19.057 | −9.822 | 1.00 | 19.42 |
| ATOM | 10250 | N | ASP | 1535 | 12.318 | −17.243 | −10.085 | 1.00 | 22.15 |
| ATOM | 10251 | CA | ASP | 1535 | 11.530 | −17.879 | −11.131 | 1.00 | 22.54 |
| ATOM | 10252 | CB | ASP | 1535 | 10.232 | −17.109 | −11.387 | 1.00 | 24.94 |
| ATOM | 10253 | CG | ASP | 1535 | 9.228 | −17.260 | −10.259 | 1.00 | 26.10 |
| ATOM | 10254 | OD1 | ASP | 1535 | 9.260 | −18.288 | −9.553 | 1.00 | 29.47 |
| ATOM | 10255 | OD2 | ASP | 1535 | 8.398 | −16.352 | −10.093 | 1.00 | 30.35 |
| ATOM | 10256 | C | ASP | 1535 | 12.287 | −18.042 | −12.447 | 1.00 | 22.47 |
| ATOM | 10257 | O | ASP | 1535 | 11.874 | −18.823 | −13.296 | 1.00 | 22.23 |
| ATOM | 10258 | N | GLU | 1536 | 13.386 | −17.311 | −12.617 | 1.00 | 22.56 |
| ATOM | 10259 | CA | GLU | 1536 | 14.187 | −17.407 | −13.838 | 1.00 | 23.73 |
| ATOM | 10260 | CB | GLU | 1536 | 14.678 | −16.024 | −14.278 | 1.00 | 23.59 |
| ATOM | 10261 | CG | GLU | 1536 | 13.583 | −15.086 | −14.756 | 1.00 | 25.82 |
| ATOM | 10262 | CD | GLU | 1536 | 12.700 | −15.718 | −15.814 | 1.00 | 26.18 |
| ATOM | 10263 | OE1 | GLU | 1536 | 13.256 | −16.349 | −16.739 | 1.00 | 25.95 |
| ATOM | 10264 | OE2 | GLU | 1536 | 11.459 | −15.575 | −15.718 | 1.00 | 26.71 |
| ATOM | 10265 | C | GLU | 1536 | 15.397 | −18.325 | −13.672 | 1.00 | 24.75 |
| ATOM | 10266 | O | GLU | 1536 | 15.973 | −18.784 | −14.654 | 1.00 | 26.28 |
| ATOM | 10267 | N | GLY | 1537 | 15.797 | −18.583 | −12.433 | 1.00 | 25.87 |
| ATOM | 10268 | CA | GLY | 1537 | 16.944 | −19.447 | −12.223 | 1.00 | 25.64 |
| ATOM | 10269 | C | GLY | 1537 | 18.037 | −18.855 | −11.356 | 1.00 | 24.92 |
| ATOM | 10270 | O | GLY | 1537 | 18.903 | −19.588 | −10.893 | 1.00 | 26.48 |
| ATOM | 10271 | N | LEU | 1538 | 18.014 | −17.540 | −11.148 | 1.00 | 23.82 |
| ATOM | 10272 | CA | LEU | 1538 | 19.010 | −16.872 | −10.305 | 1.00 | 24.22 |
| ATOM | 10273 | CB | LEU | 1538 | 18.938 | −15.356 | −10.488 | 1.00 | 24.47 |
| ATOM | 10274 | CG | LEU | 1538 | 20.087 | −14.653 | −11.205 | 1.00 | 26.59 |
| ATOM | 10275 | CD1 | LEU | 1538 | 19.769 | −13.170 | −11.298 | 1.00 | 23.00 |
| ATOM | 10276 | CD2 | LEU | 1538 | 21.403 | −14.879 | −10.479 | 1.00 | 25.88 |
| ATOM | 10277 | C | LEU | 1538 | 18.682 | −17.223 | −8.862 | 1.00 | 23.54 |
| ATOM | 10278 | O | LEU | 1538 | 17.831 | −16.580 | −8.246 | 1.00 | 22.67 |
| ATOM | 10279 | N | ASN | 1539 | 19.372 | −18.235 | −8.338 | 1.00 | 23.14 |
| ATOM | 10280 | CA | ASN | 1539 | 19.146 | −18.740 | −6.985 | 1.00 | 23.02 |
| ATOM | 10281 | CB | ASN | 1539 | 19.144 | −20.276 | −6.991 | 1.00 | 25.31 |
| ATOM | 10282 | CG | ASN | 1539 | 18.057 | −20.857 | −7.877 | 1.00 | 29.87 |
| ATOM | 10283 | OD1 | ASN | 1539 | 16.946 | −20.333 | −7.935 | 1.00 | 31.66 |
| ATOM | 10284 | ND2 | ASN | 1539 | 18.367 | −21.956 | −8.560 | 1.00 | 32.41 |
| ATOM | 10285 | C | ASN | 1539 | 20.120 | −18.266 | −5.905 | 1.00 | 21.17 |
| ATOM | 10286 | O | ASN | 1539 | 20.100 | −18.785 | −4.787 | 1.00 | 22.27 |
| ATOM | 10287 | N | VAL | 1540 | 20.973 | −17.303 | −6.229 | 1.00 | 18.20 |
| ATOM | 10288 | CA | VAL | 1540 | 21.922 | −16.778 | −5.255 | 1.00 | 17.11 |
| ATOM | 10289 | CB | VAL | 1540 | 23.383 | −17.043 | −5.672 | 1.00 | 16.57 |
| ATOM | 10290 | CG1 | VAL | 1540 | 24.322 | −16.576 | −4.558 | 1.00 | 19.33 |
| ATOM | 10291 | CG2 | VAL | 1540 | 23.583 | −18.517 | −5.941 | 1.00 | 17.44 |
| ATOM | 10292 | C | VAL | 1540 | 21.699 | −15.284 | −5.161 | 1.00 | 15.86 |
| ATOM | 10293 | O | VAL | 1540 | 21.949 | −14.537 | −6.113 | 1.00 | 14.51 |
| ATOM | 10294 | N | MET | 1541 | 21.218 | −14.845 | −4.008 | 1.00 | 14.44 |
| ATOM | 10295 | CA | MET | 1541 | 20.921 | −13.437 | −3.815 | 1.00 | 14.69 |
| ATOM | 10296 | CB | MET | 1541 | 19.408 | −13.226 | −3.696 | 1.00 | 16.00 |
| ATOM | 10297 | CG | MET | 1541 | 18.661 | −13.438 | −4.990 | 1.00 | 17.44 |
| ATOM | 10298 | SD | MET | 1541 | 16.908 | −13.405 | −4.789 | 1.00 | 18.84 |
| ATOM | 10299 | CE | MET | 1541 | 16.460 | −15.008 | −5.443 | 1.00 | 17.64 |
| ATOM | 10300 | C | MET | 1541 | 21.579 | −12.869 | −2.586 | 1.00 | 14.81 |
| ATOM | 10301 | O | MET | 1541 | 21.776 | −13.564 | −1.587 | 1.00 | 15.31 |
| ATOM | 10302 | N | LEU | 1542 | 21.885 | −11.585 | −2.656 | 1.00 | 14.00 |
| ATOM | 10303 | CA | LEU | 1542 | 22.518 | −10.920 | −1.537 | 1.00 | 13.41 |
| ATOM | 10304 | CB | LEU | 1542 | 23.945 | −10.527 | −1.929 | 1.00 | 16.33 |
| ATOM | 10305 | CG | LEU | 1542 | 24.874 | −9.753 | −0.982 | 1.00 | 22.57 |
| ATOM | 10306 | CD1 | LEU | 1542 | 24.655 | −8.278 | −1.204 | 1.00 | 25.11 |
| ATOM | 10307 | CD2 | LEU | 1542 | 24.678 | −10.149 | 0.485 | 1.00 | 19.55 |
| ATOM | 10308 | C | LEU | 1542 | 21.726 | −9.706 | −1.077 | 1.00 | 11.50 |
| ATOM | 10309 | O | LEU | 1542 | 21.406 | −8.814 | −1.862 | 1.00 | 11.99 |
| ATOM | 10310 | N | VAL | 1543 | 21.394 | −9.701 | 0.210 | 1.00 | 12.88 |
| ATOM | 10311 | CA | VAL | 1543 | 20.685 | −8.590 | 0.805 | 1.00 | 12.95 |
| ATOM | 10312 | CB | VAL | 1543 | 19.750 | −9.067 | 1.918 | 1.00 | 14.28 |
| ATOM | 10313 | CG1 | VAL | 1543 | 19.016 | −7.885 | 2.529 | 1.00 | 15.11 |
| ATOM | 10314 | CG2 | VAL | 1543 | 18.759 | −10.079 | 1.344 | 1.00 | 13.81 |
| ATOM | 10315 | C | VAL | 1543 | 21.838 | −7.785 | 1.377 | 1.00 | 14.63 |
| ATOM | 10316 | O | VAL | 1543 | 22.264 | −7.998 | 2.516 | 1.00 | 14.81 |
| ATOM | 10317 | N | GLY | 1544 | 22.362 | −6.883 | 0.557 | 1.00 | 14.42 |
| ATOM | 10318 | CA | GLY | 1544 | 23.503 | −6.088 | 0.966 | 1.00 | 16.82 |
| ATOM | 10319 | C | GLY | 1544 | 23.197 | −4.703 | 1.471 | 1.00 | 16.06 |
| ATOM | 10320 | O | GLY | 1544 | 22.122 | −4.155 | 1.207 | 1.00 | 17.57 |
| ATOM | 10321 | N | ASP | 1545 | 24.153 | −4.126 | 2.196 | 1.00 | 16.67 |
| ATOM | 10322 | CA | ASP | 1545 | 23.945 | −2.796 | 2.730 | 1.00 | 16.34 |
| ATOM | 10323 | CB | ASP | 1545 | 24.990 | −2.444 | 3.808 | 1.00 | 18.01 |
| ATOM | 10324 | CG | ASP | 1545 | 26.422 | −2.547 | 3.324 | 1.00 | 17.58 |
| ATOM | 10325 | OD1 | ASP | 1545 | 26.656 | −2.778 | 2.126 | 1.00 | 18.33 |
| ATOM | 10326 | OD2 | ASP | 1545 | 27.321 | −2.384 | 4.171 | 1.00 | 20.23 |
| ATOM | 10327 | C | ASP | 1545 | 23.910 | −1.765 | 1.613 | 1.00 | 15.12 |
| ATOM | 10328 | O | ASP | 1545 | 23.718 | −0.578 | 1.860 | 1.00 | 17.15 |
| ATOM | 10329 | N | SER | 1546 | 24.066 | −2.223 | 0.373 | 1.00 | 15.22 |
| ATOM | 10330 | CA | SER | 1546 | 23.966 | −1.307 | −0.752 | 1.00 | 13.69 |
| ATOM | 10331 | CB | SER | 1546 | 24.223 | −2.036 | −2.077 | 1.00 | 16.92 |
| ATOM | 10332 | OG | SER | 1546 | 23.495 | −3.253 | −2.157 | 1.00 | 16.30 |
| ATOM | 10333 | C | SER | 1546 | 22.554 | −0.721 | −0.725 | 1.00 | 15.02 |
| ATOM | 10334 | O | SER | 1546 | 22.302 | 0.346 | −1.289 | 1.00 | 16.38 |
| ATOM | 10335 | N | LEU | 1547 | 21.627 | −1.411 | −0.057 | 1.00 | 13.61 |
| ATOM | 10336 | CA | LEU | 1547 | 20.255 | −0.904 | 0.033 | 1.00 | 13.00 |
| ATOM | 10337 | CB | LEU | 1547 | 19.359 | −1.891 | 0.804 | 1.00 | 14.09 |
| ATOM | 10338 | CG | LEU | 1547 | 19.730 | −2.207 | 2.259 | 1.00 | 11.97 |
| ATOM | 10339 | CD1 | LEU | 1547 | 19.088 | −1.193 | 3.174 | 1.00 | 17.68 |
| ATOM | 10340 | CD2 | LEU | 1547 | 19.261 | −3.621 | 2.631 | 1.00 | 16.59 |
| ATOM | 10341 | C | LEU | 1547 | 20.246 | 0.471 | 0.711 | 1.00 | 13.72 |
| ATOM | 10342 | O | LEU | 1547 | 19.303 | 1.249 | 0.556 | 1.00 | 12.82 |
| ATOM | 10343 | N | GLY | 1548 | 21.302 | 0.768 | 1.462 | 1.00 | 14.37 |
| ATOM | 10344 | CA | GLY | 1548 | 21.386 | 2.059 | 2.128 | 1.00 | 13.78 |
| ATOM | 10345 | C | GLY | 1548 | 21.310 | 3.187 | 1.120 | 1.00 | 15.41 |
| ATOM | 10346 | O | GLY | 1548 | 20.786 | 4.267 | 1.403 | 1.00 | 15.37 |
| ATOM | 10347 | N | MET | 1549 | 21.805 | 2.914 | −0.083 | 1.00 | 16.39 |
| ATOM | 10348 | CA | MET | 1549 | 21.825 | 3.900 | −1.151 | 1.00 | 16.76 |
| ATOM | 10349 | CB | MET | 1549 | 23.198 | 3.852 | −1.842 | 1.00 | 18.82 |
| ATOM | 10350 | CG | MET | 1549 | 24.346 | 4.147 | −0.882 | 1.00 | 20.11 |
| ATOM | 10351 | SD | MET | 1549 | 26.012 | 3.963 | −1.563 | 1.00 | 26.68 |
| ATOM | 10352 | CE | MET | 1549 | 26.138 | 5.439 | −2.542 | 1.00 | 27.73 |
| ATOM | 10353 | C | MET | 1549 | 20.696 | 3.703 | −2.165 | 1.00 | 17.66 |
| ATOM | 10354 | O | MET | 1549 | 19.935 | 4.627 | −2.443 | 1.00 | 19.20 |
| ATOM | 10355 | N | THR | 1550 | 20.567 | 2.495 | −2.700 | 1.00 | 17.07 |
| ATOM | 10356 | CA | THR | 1550 | 19.529 | 2.234 | −3.693 | 1.00 | 17.56 |
| ATOM | 10357 | CB | THR | 1550 | 19.809 | 0.931 | −4.439 | 1.00 | 21.36 |
| ATOM | 10358 | OG1 | THR | 1550 | 18.737 | 0.665 | −5.349 | 1.00 | 29.04 |
| ATOM | 10359 | CG2 | THR | 1550 | 19.953 | −0.219 | −3.472 | 1.00 | 19.69 |
| ATOM | 10360 | C | THR | 1550 | 18.103 | 2.191 | −3.143 | 1.00 | 17.30 |
| ATOM | 10361 | O | THR | 1550 | 17.144 | 2.507 | −3.853 | 1.00 | 19.51 |
| ATOM | 10362 | N | VAL | 1551 | 17.949 | 1.800 | −1.884 | 1.00 | 16.88 |
| ATOM | 10363 | CA | VAL | 1551 | 16.616 | 1.741 | −1.297 | 1.00 | 15.66 |
| ATOM | 10364 | CB | VAL | 1551 | 16.412 | 0.423 | −0.504 | 1.00 | 16.16 |
| ATOM | 10365 | CG1 | VAL | 1551 | 15.077 | 0.454 | 0.240 | 1.00 | 15.49 |
| ATOM | 10366 | CG2 | VAL | 1551 | 16.436 | −0.760 | −1.452 | 1.00 | 17.32 |
| ATOM | 10367 | C | VAL | 1551 | 16.344 | 2.929 | −0.377 | 1.00 | 14.94 |
| ATOM | 10368 | O | VAL | 1551 | 15.351 | 3.635 | −0.540 | 1.00 | 13.06 |
| ATOM | 10369 | N | GLN | 1552 | 17.231 | 3.155 | 0.584 | 1.00 | 13.60 |
| ATOM | 10370 | CA | GLN | 1552 | 17.041 | 4.242 | 1.536 | 1.00 | 13.62 |
| ATOM | 10371 | CB | GLN | 1552 | 17.857 | 3.963 | 2.798 | 1.00 | 14.36 |
| ATOM | 10372 | CG | GLN | 1552 | 17.589 | 2.569 | 3.329 | 1.00 | 15.64 |
| ATOM | 10373 | CD | GLN | 1552 | 18.298 | 2.290 | 4.628 | 1.00 | 14.11 |
| ATOM | 10374 | OE1 | GLN | 1552 | 19.342 | 2.875 | 4.914 | 1.00 | 17.01 |
| ATOM | 10375 | NE2 | GLN | 1552 | 17.744 | 1.372 | 5.418 | 1.00 | 12.68 |
| ATOM | 10376 | C | GLN | 1552 | 17.396 | 5.610 | 0.968 | 1.00 | 12.37 |
| ATOM | 10377 | O | GLN | 1552 | 16.852 | 6.625 | 1.403 | 1.00 | 13.34 |
| ATOM | 10378 | N | GLY | 1553 | 18.312 | 5.643 | 0.007 | 1.00 | 13.54 |
| ATOM | 10379 | CA | GLY | 1553 | 18.679 | 6.914 | −0.585 | 1.00 | 12.70 |
| ATOM | 10380 | C | GLY | 1553 | 19.761 | 7.687 | 0.140 | 1.00 | 16.01 |
| ATOM | 10381 | O | GLY | 1553 | 19.859 | 8.905 | −0.013 | 1.00 | 14.75 |
| ATOM | 10382 | N | HIS | 1554 | 20.563 | 6.999 | 0.948 | 1.00 | 16.13 |
| ATOM | 10383 | CA | HIS | 1554 | 21.659 | 7.665 | 1.655 | 1.00 | 19.25 |
| ATOM | 10384 | CB | HIS | 1554 | 22.058 | 6.870 | 2.901 | 1.00 | 18.68 |
| ATOM | 10385 | CG | HIS | 1554 | 21.002 | 6.837 | 3.962 | 1.00 | 18.56 |
| ATOM | 10386 | CD2 | HIS | 1554 | 20.261 | 5.814 | 4.452 | 1.00 | 18.97 |
| ATOM | 10387 | ND1 | HIS | 1554 | 20.599 | 7.964 | 4.646 | 1.00 | 19.57 |
| ATOM | 10388 | CE1 | HIS | 1554 | 19.654 | 7.637 | 5.509 | 1.00 | 21.71 |
| ATOM | 10389 | NE2 | HIS | 1554 | 19.429 | 6.338 | 5.412 | 1.00 | 16.82 |
| ATOM | 10390 | C | HIS | 1554 | 22.845 | 7.761 | 0.692 | 1.00 | 20.59 |
| ATOM | 10391 | O | HIS | 1554 | 22.867 | 7.081 | −0.332 | 1.00 | 20.01 |
| ATOM | 10392 | N | ASP | 1555 | 23.824 | 8.604 | 1.023 | 1.00 | 23.52 |
| ATOM | 10393 | CA | ASP | 1555 | 25.007 | 8.786 | 0.182 | 1.00 | 25.86 |
| ATOM | 10394 | CB | ASP | 1555 | 25.650 | 10.153 | 0.451 | 1.00 | 31.31 |
| ATOM | 10395 | CG | ASP | 1555 | 25.964 | 10.375 | 1.919 | 1.00 | 34.15 |
| ATOM | 10396 | OD1 | ASP | 1555 | 26.709 | 9.570 | 2.509 | 1.00 | 37.32 |
| ATOM | 10397 | OD2 | ASP | 1555 | 25.461 | 11.366 | 2.489 | 1.00 | 40.85 |
| ATOM | 10398 | C | ASP | 1555 | 26.055 | 7.695 | 0.374 | 1.00 | 25.31 |
| ATOM | 10399 | O | ASP | 1555 | 27.045 | 7.637 | −0.356 | 1.00 | 27.92 |
| ATOM | 10400 | N | SER | 1556 | 25.841 | 6.832 | 1.359 | 1.00 | 21.86 |
| ATOM | 10401 | CA | SER | 1556 | 26.769 | 5.742 | 1.627 | 1.00 | 19.58 |
| ATOM | 10402 | CB | SER | 1556 | 27.890 | 6.195 | 2.569 | 1.00 | 18.25 |
| ATOM | 10403 | OG | SER | 1556 | 27.408 | 6.452 | 3.880 | 1.00 | 19.02 |
| ATOM | 10404 | C | SER | 1556 | 25.987 | 4.608 | 2.264 | 1.00 | 18.25 |
| ATOM | 10405 | O | SER | 1556 | 24.791 | 4.739 | 2.491 | 1.00 | 16.30 |
| ATOM | 10406 | N | THR | 1557 | 26.665 | 3.505 | 2.560 | 1.00 | 16.59 |
| ATOM | 10407 | CA | THR | 1557 | 26.011 | 2.353 | 3.156 | 1.00 | 16.89 |
| ATOM | 10408 | CB | THR | 1557 | 26.639 | 1.033 | 2.664 | 1.00 | 17.09 |
| ATOM | 10409 | OG1 | THR | 1557 | 27.965 | 0.921 | 3.192 | 1.00 | 17.06 |
| ATOM | 10410 | CG2 | THR | 1557 | 26.698 | 0.996 | 1.134 | 1.00 | 17.62 |
| ATOM | 10411 | C | THR | 1557 | 26.079 | 2.350 | 4.682 | 1.00 | 16.25 |
| ATOM | 10412 | O | THR | 1557 | 25.424 | 1.530 | 5.315 | 1.00 | 16.54 |
| ATOM | 10413 | N | LEU | 1558 | 26.858 | 3.257 | 5.274 | 1.00 | 15.68 |
| ATOM | 10414 | CA | LEU | 1558 | 27.018 | 3.301 | 6.738 | 1.00 | 16.74 |
| ATOM | 10415 | CB | LEU | 1558 | 27.954 | 4.450 | 7.141 | 1.00 | 17.19 |
| ATOM | 10416 | CG | LEU | 1558 | 29.465 | 4.181 | 7.061 | 1.00 | 17.43 |
| ATOM | 10417 | CD1 | LEU | 1558 | 29.864 | 3.894 | 5.620 | 1.00 | 17.48 |
| ATOM | 10418 | CD2 | LEU | 1558 | 30.223 | 5.395 | 7.599 | 1.00 | 18.51 |
| ATOM | 10419 | C | LEU | 1558 | 25.747 | 3.376 | 7.596 | 1.00 | 15.47 |
| ATOM | 10420 | O | LEU | 1558 | 25.676 | 2.770 | 8.666 | 1.00 | 14.85 |
| ATOM | 10421 | N | PRO | 1559 | 24.736 | 4.134 | 7.156 | 1.00 | 16.50 |
| ATOM | 10422 | CD | PRO | 1559 | 24.743 | 5.128 | 6.072 | 1.00 | 15.31 |
| ATOM | 10423 | CA | PRO | 1559 | 23.507 | 4.227 | 7.951 | 1.00 | 15.63 |
| ATOM | 10424 | CB | PRO | 1559 | 22.736 | 5.338 | 7.271 | 1.00 | 18.98 |
| ATOM | 10425 | CG | PRO | 1559 | 23.279 | 5.362 | 5.865 | 1.00 | 21.76 |
| ATOM | 10426 | C | PRO | 1559 | 22.685 | 2.940 | 8.055 | 1.00 | 15.29 |
| ATOM | 10427 | O | PRO | 1559 | 21.786 | 2.840 | 8.894 | 1.00 | 16.41 |
| ATOM | 10428 | N | VAL | 1560 | 22.988 | 1.951 | 7.223 | 1.00 | 14.37 |
| ATOM | 10429 | CA | VAL | 1560 | 22.236 | 0.703 | 7.263 | 1.00 | 13.86 |
| ATOM | 10430 | CB | VAL | 1560 | 22.636 | −0.220 | 6.088 | 1.00 | 13.89 |
| ATOM | 10431 | CG1 | VAL | 1560 | 21.928 | −1.553 | 6.199 | 1.00 | 15.90 |
| ATOM | 10432 | CG2 | VAL | 1560 | 22.293 | 0.446 | 4.773 | 1.00 | 12.67 |
| ATOM | 10433 | C | VAL | 1560 | 22.500 | −0.025 | 8.579 | 1.00 | 13.66 |
| ATOM | 10434 | O | VAL | 1560 | 23.653 | −0.180 | 8.993 | 1.00 | 12.04 |
| ATOM | 10435 | N | THR | 1561 | 21.448 | −0.467 | 9.254 | 1.00 | 14.89 |
| ATOM | 10436 | CA | THR | 1561 | 21.676 | −1.191 | 10.500 | 1.00 | 18.92 |
| ATOM | 10437 | CB | THR | 1561 | 21.076 | −0.416 | 11.713 | 1.00 | 25.70 |
| ATOM | 10438 | OG1 | THR | 1561 | 22.006 | 0.601 | 12.128 | 1.00 | 30.54 |
| ATOM | 10439 | CG2 | THR | 1561 | 20.806 | −1.340 | 12.899 | 1.00 | 27.97 |
| ATOM | 10440 | C | THR | 1561 | 21.179 | −2.632 | 10.388 | 1.00 | 17.00 |
| ATOM | 10441 | O | THR | 1561 | 20.535 | −2.998 | 9.405 | 1.00 | 13.03 |
| ATOM | 10442 | N | VAL | 1562 | 21.516 | −3.458 | 11.375 | 1.00 | 14.93 |
| ATOM | 10443 | CA | VAL | 1562 | 21.134 | −4.868 | 11.378 | 1.00 | 13.72 |
| ATOM | 10444 | CB | VAL | 1562 | 21.617 | −5.554 | 12.691 | 1.00 | 14.94 |
| ATOM | 10445 | CG1 | VAL | 1562 | 21.157 | −7.006 | 12.747 | 1.00 | 13.33 |
| ATOM | 10446 | CG2 | VAL | 1562 | 23.136 | −5.481 | 12.760 | 1.00 | 15.87 |
| ATOM | 10447 | C | VAL | 1562 | 19.633 | −5.044 | 11.218 | 1.00 | 13.79 |
| ATOM | 10448 | O | VAL | 1562 | 19.184 | −5.919 | 10.477 | 1.00 | 11.90 |
| ATOM | 10449 | N | ALA | 1563 | 18.862 | −4.205 | 11.906 | 1.00 | 12.57 |
| ATOM | 10450 | CA | ALA | 1563 | 17.410 | −4.266 | 11.818 | 1.00 | 13.52 |
| ATOM | 10451 | CB | ALA | 1563 | 16.793 | −3.175 | 12.665 | 1.00 | 15.49 |
| ATOM | 10452 | C | ALA | 1563 | 16.967 | −4.106 | 10.367 | 1.00 | 13.22 |
| ATOM | 10453 | O | ALA | 1563 | 16.055 | −4.796 | 9.905 | 1.00 | 11.97 |
| ATOM | 10454 | N | ASP | 1564 | 17.603 | −3.185 | 9.649 | 1.00 | 13.76 |
| ATOM | 10455 | CA | ASP | 1564 | 17.258 | −2.973 | 8.243 | 1.00 | 14.61 |
| ATOM | 10456 | CB | ASP | 1564 | 18.111 | −1.873 | 7.601 | 1.00 | 12.63 |
| ATOM | 10457 | CG | ASP | 1564 | 17.836 | −0.491 | 8.172 | 1.00 | 15.27 |
| ATOM | 10458 | OD1 | ASP | 1564 | 16.732 | −0.262 | 8.711 | 1.00 | 12.78 |
| ATOM | 10459 | OD2 | ASP | 1564 | 18.735 | 0.359 | 8.042 | 1.00 | 13.15 |
| ATOM | 10460 | C | ASP | 1564 | 17.501 | −4.243 | 7.448 | 1.00 | 13.99 |
| ATOM | 10461 | O | ASP | 1564 | 16.647 | −4.696 | 6.685 | 1.00 | 12.31 |
| ATOM | 10462 | N | ILE | 1565 | 18.696 | −4.802 | 7.602 | 1.00 | 12.52 |
| ATOM | 10463 | CA | ILE | 1565 | 19.042 | −6.019 | 6.878 | 1.00 | 11.04 |
| ATOM | 10464 | CB | ILE | 1565 | 20.450 | −6.514 | 7.251 | 1.00 | 12.44 |
| ATOM | 10465 | CG2 | ILE | 1565 | 20.699 | −7.867 | 6.594 | 1.00 | 12.14 |
| ATOM | 10466 | CG1 | ILE | 1565 | 21.501 | −5.478 | 6.817 | 1.00 | 9.14 |
| ATOM | 10467 | CD1 | ILE | 1565 | 21.658 | −5.322 | 5.303 | 1.00 | 13.72 |
| ATOM | 10468 | C | ILE | 1565 | 18.034 | −7.132 | 7.160 | 1.00 | 11.12 |
| ATOM | 10469 | O | ILE | 1565 | 17.606 | −7.822 | 6.247 | 1.00 | 10.33 |
| ATOM | 10470 | N | ALA | 1566 | 17.651 | −7.309 | 8.422 | 1.00 | 9.51 |
| ATOM | 10471 | CA | ALA | 1566 | 16.699 | −8.364 | 8.743 | 1.00 | 10.56 |
| ATOM | 10472 | CB | ALA | 1566 | 16.495 | −8.460 | 10.241 | 1.00 | 10.34 |
| ATOM | 10473 | C | ALA | 1566 | 15.365 | −8.141 | 8.050 | 1.00 | 8.48 |
| ATOM | 10474 | O | ALA | 1566 | 14.744 | −9.084 | 7.553 | 1.00 | 10.65 |
| ATOM | 10475 | N | TYR | 1567 | 14.919 | −6.894 | 8.028 | 1.00 | 10.49 |
| ATOM | 10476 | CA | TYR | 1567 | 13.644 | −6.538 | 7.394 | 1.00 | 10.89 |
| ATOM | 10477 | CB | TYR | 1567 | 13.426 | −5.023 | 7.478 | 1.00 | 11.37 |
| ATOM | 10478 | CG | TYR | 1567 | 12.216 | −4.515 | 6.708 | 1.00 | 13.66 |
| ATOM | 10479 | CD1 | TYR | 1567 | 10.925 | −4.761 | 7.163 | 1.00 | 12.46 |
| ATOM | 10480 | CE1 | TYR | 1567 | 9.816 | −4.222 | 6.513 | 1.00 | 14.53 |
| ATOM | 10481 | CD2 | TYR | 1567 | 12.373 | −3.724 | 5.568 | 1.00 | 15.00 |
| ATOM | 10482 | CE2 | TYR | 1567 | 11.277 | −3.180 | 4.909 | 1.00 | 14.51 |
| ATOM | 10483 | CZ | TYR | 1567 | 10.002 | −3.429 | 5.391 | 1.00 | 14.84 |
| ATOM | 10484 | OH | TYR | 1567 | 8.917 | −2.852 | 4.784 | 1.00 | 13.91 |
| ATOM | 10485 | C | TYR | 1567 | 13.639 | −6.960 | 5.933 | 1.00 | 10.89 |
| ATOM | 10486 | O | TYR | 1567 | 12.708 | −7.617 | 5.460 | 1.00 | 10.65 |
| ATOM | 10487 | N | HIS | 1568 | 14.682 | −6.558 | 5.217 | 1.00 | 10.43 |
| ATOM | 10488 | CA | HIS | 1568 | 14.775 | −6.881 | 3.801 | 1.00 | 10.21 |
| ATOM | 10489 | CB | HIS | 1568 | 15.842 | −5.998 | 3.140 | 1.00 | 11.31 |
| ATOM | 10490 | CG | HIS | 1568 | 15.411 | −4.565 | 2.971 | 1.00 | 11.56 |
| ATOM | 10491 | CD2 | HIS | 1568 | 15.646 | −3.467 | 3.731 | 1.00 | 11.51 |
| ATOM | 10492 | ND1 | HIS | 1568 | 14.579 | −4.149 | 1.953 | 1.00 | 12.16 |
| ATOM | 10493 | CE1 | HIS | 1568 | 14.319 | −2.861 | 2.091 | 1.00 | 13.74 |
| ATOM | 10494 | NE2 | HIS | 1568 | 14.954 | −2.422 | 3.163 | 1.00 | 12.66 |
| ATOM | 10495 | C | HIS | 1568 | 15.044 | −8.370 | 3.594 | 1.00 | 11.46 |
| ATOM | 10496 | O | HIS | 1568 | 14.540 | −8.977 | 2.643 | 1.00 | 10.22 |
| ATOM | 10497 | N | THR | 1569 | 15.819 | −8.970 | 4.492 | 1.00 | 10.71 |
| ATOM | 10498 | CA | THR | 1569 | 16.104 | −10.397 | 4.390 | 1.00 | 10.69 |
| ATOM | 10499 | CB | THR | 1569 | 17.054 | −10.853 | 5.521 | 1.00 | 12.47 |
| ATOM | 10500 | OG1 | THR | 1569 | 18.347 | −10.273 | 5.306 | 1.00 | 10.54 |
| ATOM | 10501 | CG2 | THR | 1569 | 17.169 | −12.392 | 5.559 | 1.00 | 11.72 |
| ATOM | 10502 | C | THR | 1569 | 14.822 | −11.236 | 4.436 | 1.00 | 12.77 |
| ATOM | 10503 | O | THR | 1569 | 14.657 | −12.170 | 3.646 | 1.00 | 11.27 |
| ATOM | 10504 | N | ALA | 1570 | 13.915 | −10.915 | 5.359 | 1.00 | 11.72 |
| ATOM | 10505 | CA | ALA | 1570 | 12.666 | −11.671 | 5.471 | 1.00 | 13.45 |
| ATOM | 10506 | CB | ALA | 1570 | 11.861 | −11.200 | 6.689 | 1.00 | 12.98 |
| ATOM | 10507 | C | ALA | 1570 | 11.819 | −11.544 | 4.211 | 1.00 | 12.73 |
| ATOM | 10508 | O | ALA | 1570 | 11.182 | −12.506 | 3.784 | 1.00 | 13.55 |
| ATOM | 10509 | N | ALA | 1571 | 11.804 | −10.348 | 3.628 | 1.00 | 12.25 |
| ATOM | 10510 | CA | ALA | 1571 | 11.028 | −10.110 | 2.422 | 1.00 | 10.68 |
| ATOM | 10511 | CB | ALA | 1571 | 11.014 | −8.619 | 2.086 | 1.00 | 10.51 |
| ATOM | 10512 | C | ALA | 1571 | 11.615 | −10.907 | 1.262 | 1.00 | 11.99 |
| ATOM | 10513 | O | ALA | 1571 | 10.881 | −11.499 | 0.477 | 1.00 | 11.47 |
| ATOM | 10514 | N | VAL | 1572 | 12.939 | −10.923 | 1.144 | 1.00 | 13.05 |
| ATOM | 10515 | CA | VAL | 1572 | 13.553 | −11.687 | 0.064 | 1.00 | 13.95 |
| ATOM | 10516 | CB | VAL | 1572 | 15.075 | −11.473 | 0.004 | 1.00 | 13.81 |
| ATOM | 10517 | CG1 | VAL | 1572 | 15.701 | −12.485 | −0.954 | 1.00 | 14.45 |
| ATOM | 10518 | CG2 | VAL | 1572 | 15.379 | −10.050 | −0.458 | 1.00 | 14.01 |
| ATOM | 10519 | C | VAL | 1572 | 13.271 | −13.178 | 0.239 | 1.00 | 14.25 |
| ATOM | 10520 | O | VAL | 1572 | 12.956 | −13.888 | −0.727 | 1.00 | 14.24 |
| ATOM | 10521 | N | ARG | 1573 | 13.388 | −13.659 | 1.471 | 1.00 | 13.28 |
| ATOM | 10522 | CA | ARG | 1573 | 13.137 | −15.066 | 1.733 | 1.00 | 12.96 |
| ATOM | 10523 | CB | ARG | 1573 | 13.418 | −15.409 | 3.208 | 1.00 | 12.40 |
| ATOM | 10524 | CG | ARG | 1573 | 13.134 | −16.875 | 3.564 | 1.00 | 14.12 |
| ATOM | 10525 | CD | ARG | 1573 | 13.891 | −17.820 | 2.636 | 1.00 | 11.99 |
| ATOM | 10526 | NE | ARG | 1573 | 15.299 | −17.944 | 3.009 | 1.00 | 15.31 |
| ATOM | 10527 | CZ | ARG | 1573 | 16.238 | −18.497 | 2.248 | 1.00 | 17.73 |
| ATOM | 10528 | NH1 | ARG | 1573 | 15.931 | −18.981 | 1.048 | 1.00 | 18.85 |
| ATOM | 10529 | NH2 | ARG | 1573 | 17.486 | −18.589 | 2.696 | 1.00 | 15.43 |
| ATOM | 10530 | C | ARG | 1573 | 11.701 | −15.460 | 1.358 | 1.00 | 12.91 |
| ATOM | 10531 | O | ARG | 1573 | 11.467 | −16.569 | 0.882 | 1.00 | 14.66 |
| ATOM | 10532 | N | ARG | 1574 | 10.740 | −14.561 | 1.559 | 1.00 | 15.46 |
| ATOM | 10533 | CA | ARG | 1574 | 9.356 | −14.883 | 1.214 | 1.00 | 13.92 |
| ATOM | 10534 | CB | ARG | 1574 | 8.392 | −13.802 | 1.707 | 1.00 | 14.87 |
| ATOM | 10535 | CG | ARG | 1574 | 8.358 | −13.622 | 3.212 | 1.00 | 17.41 |
| ATOM | 10536 | CD | ARG | 1574 | 7.182 | −12.767 | 3.655 | 1.00 | 17.23 |
| ATOM | 10537 | NE | ARG | 1574 | 7.241 | −12.534 | 5.096 | 1.00 | 19.59 |
| ATOM | 10538 | CZ | ARG | 1574 | 7.851 | −11.505 | 5.673 | 1.00 | 20.15 |
| ATOM | 10539 | NH1 | ARG | 1574 | 8.459 | −10.577 | 4.939 | 1.00 | 17.97 |
| ATOM | 10540 | NH2 | ARG | 1574 | 7.882 | −11.421 | 6.995 | 1.00 | 23.22 |
| ATOM | 10541 | C | ARG | 1574 | 9.210 | −15.013 | −0.294 | 1.00 | 13.49 |
| ATOM | 10542 | O | ARG | 1574 | 8.380 | −15.777 | −0.786 | 1.00 | 14.10 |
| ATOM | 10543 | N | GLY | 1575 | 10.017 | −14.256 | −1.024 | 1.00 | 12.47 |
| ATOM | 10544 | CA | GLY | 1575 | 9.949 | −14.297 | −2.474 | 1.00 | 15.24 |
| ATOM | 10545 | C | GLY | 1575 | 10.693 | −15.461 | −3.096 | 1.00 | 16.10 |
| ATOM | 10546 | O | GLY | 1575 | 10.307 | −15.961 | −4.150 | 1.00 | 17.71 |
| ATOM | 10547 | N | ALA | 1576 | 11.761 | −15.894 | −2.441 | 1.00 | 15.51 |
| ATOM | 10548 | CA | ALA | 1576 | 12.577 | −16.990 | −2.950 | 1.00 | 16.76 |
| ATOM | 10549 | CB | ALA | 1576 | 13.834 | −16.430 | −3.584 | 1.00 | 18.02 |
| ATOM | 10550 | C | ALA | 1576 | 12.935 | −17.923 | −1.800 | 1.00 | 16.86 |
| ATOM | 10551 | O | ALA | 1576 | 14.065 | −17.923 | −1.318 | 1.00 | 17.30 |
| ATOM | 10552 | N | PRO | 1577 | 11.977 | −18.756 | −1.368 | 1.00 | 18.36 |
| ATOM | 10553 | CD | PRO | 1577 | 10.647 | −18.935 | −1.980 | 1.00 | 17.74 |
| ATOM | 10554 | CA | PRO | 1577 | 12.176 | −19.696 | −0.262 | 1.00 | 19.18 |
| ATOM | 10555 | CB | PRO | 1577 | 10.783 | −20.287 | −0.063 | 1.00 | 19.74 |
| ATOM | 10556 | CG | PRO | 1577 | 10.218 | −20.291 | −1.442 | 1.00 | 22.52 |
| ATOM | 10557 | C | PRO | 1577 | 13.255 | −20.767 | −0.421 | 1.00 | 21.10 |
| ATOM | 10558 | O | PRO | 1577 | 13.664 | −21.380 | 0.566 | 1.00 | 21.21 |
| ATOM | 10559 | N | ASN | 1578 | 13.725 | −20.987 | −1.647 | 1.00 | 19.12 |
| ATOM | 10560 | CA | ASN | 1578 | 14.741 | −22.008 | −1.879 | 1.00 | 19.33 |
| ATOM | 10561 | CB | ASN | 1578 | 14.237 | −23.029 | −2.898 | 1.00 | 23.93 |
| ATOM | 10562 | CG | ASN | 1578 | 12.980 | −23.737 | −2.435 | 1.00 | 26.55 |
| ATOM | 10563 | OD1 | ASN | 1578 | 12.942 | −24.304 | −1.344 | 1.00 | 30.38 |
| ATOM | 10564 | ND2 | ASN | 1578 | 11.942 | −23.707 | −3.266 | 1.00 | 30.04 |
| ATOM | 10565 | C | ASN | 1578 | 16.077 | −21.465 | −2.347 | 1.00 | 18.42 |
| ATOM | 10566 | O | ASN | 1578 | 16.972 | −22.230 | −2.711 | 1.00 | 17.87 |
| ATOM | 10567 | N | CYS | 1579 | 16.230 | −20.148 | −2.333 | 1.00 | 16.75 |
| ATOM | 10568 | CA | CYS | 1579 | 17.486 | −19.565 | −2.794 | 1.00 | 16.63 |
| ATOM | 10569 | CB | CYS | 1579 | 17.268 | −18.119 | −3.256 | 1.00 | 18.58 |
| ATOM | 10570 | SG | CYS | 1579 | 17.273 | −16.870 | −1.911 | 1.00 | 20.66 |
| ATOM | 10571 | C | CYS | 1579 | 18.550 | −19.569 | −1.708 | 1.00 | 16.46 |
| ATOM | 10572 | O | CYS | 1579 | 18.254 | −19.737 | −0.523 | 1.00 | 16.94 |
| ATOM | 10573 | N | LEU | 1580 | 19.797 | −19.406 | −2.127 | 1.00 | 16.30 |
| ATOM | 10574 | CA | LEU | 1580 | 20.901 | −19.306 | −1.183 | 1.00 | 16.34 |
| ATOM | 10575 | CB | LEU | 1580 | 22.225 | −19.741 | −1.820 | 1.00 | 16.18 |
| ATOM | 10576 | CG | LEU | 1580 | 23.496 | −19.451 | −1.005 | 1.00 | 16.78 |
| ATOM | 10577 | CD1 | LEU | 1580 | 23.455 | −20.123 | 0.360 | 1.00 | 18.05 |
| ATOM | 10578 | CD2 | LEU | 1580 | 24.697 | −19.931 | −1.797 | 1.00 | 19.94 |
| ATOM | 10579 | C | LEU | 1580 | 20.894 | −17.803 | −0.914 | 1.00 | 16.05 |
| ATOM | 10580 | O | LEU | 1580 | 21.150 | −16.998 | −1.813 | 1.00 | 13.83 |
| ATOM | 10581 | N | LEU | 1581 | 20.568 | −17.437 | 0.321 | 1.00 | 16.04 |
| ATOM | 10582 | CA | LEU | 1581 | 20.455 | −16.039 | 0.704 | 1.00 | 15.99 |
| ATOM | 10583 | CB | LEU | 1581 | 19.135 | −15.812 | 1.472 | 1.00 | 16.23 |
| ATOM | 10584 | CG | LEU | 1581 | 18.506 | −14.413 | 1.600 | 1.00 | 17.05 |
| ATOM | 10585 | CD1 | LEU | 1581 | 17.114 | −14.552 | 2.230 | 1.00 | 16.42 |
| ATOM | 10586 | CD2 | LEU | 1581 | 19.380 | −13.487 | 2.444 | 1.00 | 19.60 |
| ATOM | 10587 | C | LEU | 1581 | 21.617 | −15.583 | 1.569 | 1.00 | 16.66 |
| ATOM | 10588 | O | LEU | 1581 | 21.781 | −16.063 | 2.684 | 1.00 | 15.52 |
| ATOM | 10589 | N | LEU | 1582 | 22.428 | −14.679 | 1.032 | 1.00 | 15.45 |
| ATOM | 10590 | CA | LEU | 1582 | 23.536 | −14.105 | 1.761 | 1.00 | 16.37 |
| ATOM | 10591 | CB | LEU | 1582 | 24.732 | −13.839 | 0.850 | 1.00 | 17.30 |
| ATOM | 10592 | CG | LEU | 1582 | 25.836 | −14.894 | 0.746 | 1.00 | 18.08 |
| ATOM | 10593 | CD1 | LEU | 1582 | 25.274 | −16.177 | 0.172 | 1.00 | 19.04 |
| ATOM | 10594 | CD2 | LEU | 1582 | 26.966 | −14.348 | −0.130 | 1.00 | 20.45 |
| ATOM | 10595 | C | LEU | 1582 | 23.032 | −12.769 | 2.290 | 1.00 | 16.32 |
| ATOM | 10596 | O | LEU | 1582 | 22.308 | −12.046 | 1.593 | 1.00 | 18.36 |
| ATOM | 10597 | N | ALA | 1583 | 23.399 | −12.447 | 3.521 | 1.00 | 15.51 |
| ATOM | 10598 | CA | ALA | 1583 | 22.999 | −11.183 | 4.111 | 1.00 | 14.69 |
| ATOM | 10599 | CB | ALA | 1583 | 21.994 | −11.416 | 5.219 | 1.00 | 14.69 |
| ATOM | 10600 | C | ALA | 1583 | 24.234 | −10.491 | 4.667 | 1.00 | 14.14 |
| ATOM | 10601 | O | ALA | 1583 | 25.051 | −11.112 | 5.344 | 1.00 | 13.73 |
| ATOM | 10602 | N | ASP | 1584 | 24.376 | −9.207 | 4.362 | 1.00 | 13.35 |
| ATOM | 10603 | CA | ASP | 1584 | 25.505 | −8.429 | 4.855 | 1.00 | 14.49 |
| ATOM | 10604 | CB | ASP | 1584 | 25.647 | −7.117 | 4.091 | 1.00 | 15.94 |
| ATOM | 10605 | CG | ASP | 1584 | 26.458 | −7.241 | 2.830 | 1.00 | 17.35 |
| ATOM | 10606 | OD1 | ASP | 1584 | 27.193 | −8.226 | 2.661 | 1.00 | 20.03 |
| ATOM | 10607 | OD2 | ASP | 1584 | 26.357 | −6.311 | 2.006 | 1.00 | 18.72 |
| ATOM | 10608 | C | ASP | 1584 | 25.332 | −8.032 | 6.304 | 1.00 | 13.45 |
| ATOM | 10609 | O | ASP | 1584 | 24.219 | −7.753 | 6.739 | 1.00 | 12.56 |
| ATOM | 10610 | N | LEU | 1585 | 26.427 | −8.040 | 7.056 | 1.00 | 12.65 |
| ATOM | 10611 | CA | LEU | 1585 | 26.381 | −7.514 | 8.410 | 1.00 | 13.11 |
| ATOM | 10612 | CB | LEU | 1585 | 27.360 | −8.227 | 9.356 | 1.00 | 13.48 |
| ATOM | 10613 | CG | LEU | 1585 | 26.969 | −9.646 | 9.777 | 1.00 | 13.68 |
| ATOM | 10614 | CD1 | LEU | 1585 | 27.863 | −10.134 | 10.933 | 1.00 | 12.03 |
| ATOM | 10615 | CD2 | LEU | 1585 | 25.504 | −9.664 | 10.207 | 1.00 | 14.07 |
| ATOM | 10616 | C | LEU | 1585 | 26.877 | −6.102 | 8.080 | 1.00 | 15.51 |
| ATOM | 10617 | O | LEU | 1585 | 27.954 | −5.926 | 7.483 | 1.00 | 15.43 |
| ATOM | 10618 | N | PRO | 1586 | 26.083 | −5.082 | 8.422 | 1.00 | 14.53 |
| ATOM | 10619 | CD | PRO | 1586 | 24.747 | −5.205 | 9.030 | 1.00 | 16.55 |
| ATOM | 10620 | CA | PRO | 1586 | 26.413 | −3.681 | 8.163 | 1.00 | 14.80 |
| ATOM | 10621 | CB | PRO | 1586 | 25.078 | −2.974 | 8.385 | 1.00 | 14.42 |
| ATOM | 10622 | CG | PRO | 1586 | 24.459 | −3.794 | 9.483 | 1.00 | 15.58 |
| ATOM | 10623 | C | PRO | 1586 | 27.540 | −3.066 | 8.993 | 1.00 | 15.36 |
| ATOM | 10624 | O | PRO | 1586 | 28.135 | −3.709 | 9.864 | 1.00 | 13.48 |
| ATOM | 10625 | N | PHE | 1587 | 27.808 | −1.799 | 8.695 | 1.00 | 13.29 |
| ATOM | 10626 | CA | PHE | 1587 | 28.831 | −1.006 | 9.359 | 1.00 | 15.40 |
| ATOM | 10627 | CB | PHE | 1587 | 28.626 | 0.466 | 8.995 | 1.00 | 15.27 |
| ATOM | 10628 | CG | PHE | 1587 | 29.456 | 1.415 | 9.808 | 1.00 | 16.43 |
| ATOM | 10629 | CD1 | PHE | 1587 | 30.845 | 1.346 | 9.776 | 1.00 | 15.91 |
| ATOM | 10630 | CD2 | PHE | 1587 | 28.848 | 2.378 | 10.610 | 1.00 | 16.81 |
| ATOM | 10631 | CE1 | PHE | 1587 | 31.619 | 2.215 | 10.533 | 1.00 | 19.06 |
| ATOM | 10632 | CE2 | PHE | 1587 | 29.619 | 3.257 | 11.374 | 1.00 | 18.87 |
| ATOM | 10633 | CZ | PHE | 1587 | 31.009 | 3.174 | 11.332 | 1.00 | 18.40 |
| ATOM | 10634 | C | PHE | 1587 | 28.804 | −1.176 | 10.875 | 1.00 | 14.93 |
| ATOM | 10635 | O | PHE | 1587 | 27.783 | −0.954 | 11.513 | 1.00 | 13.77 |
| ATOM | 10636 | N | MET | 1588 | 29.944 | −1.580 | 11.431 | 1.00 | 16.41 |
| ATOM | 10637 | CA | MET | 1588 | 30.115 | −1.783 | 12.866 | 1.00 | 15.60 |
| ATOM | 10638 | CB | MET | 1588 | 29.958 | −0.452 | 13.610 | 1.00 | 15.47 |
| ATOM | 10639 | CG | MET | 1588 | 30.753 | −0.398 | 14.899 | 1.00 | 17.28 |
| ATOM | 10640 | SD | MET | 1588 | 32.552 | −0.481 | 14.654 | 1.00 | 16.90 |
| ATOM | 10641 | CE | MET | 1588 | 32.900 | 1.224 | 14.358 | 1.00 | 19.88 |
| ATOM | 10642 | C | MET | 1588 | 29.182 | −2.825 | 13.480 | 1.00 | 14.81 |
| ATOM | 10643 | O | MET | 1588 | 28.832 | −2.742 | 14.659 | 1.00 | 16.79 |
| ATOM | 10644 | N | ALA | 1589 | 28.782 | −3.809 | 12.689 | 1.00 | 13.82 |
| ATOM | 10645 | CA | ALA | 1589 | 27.901 | −4.853 | 13.196 | 1.00 | 14.09 |
| ATOM | 10646 | CB | ALA | 1589 | 26.873 | −5.228 | 12.123 | 1.00 | 14.61 |
| ATOM | 10647 | C | ALA | 1589 | 28.707 | −6.079 | 13.617 | 1.00 | 14.92 |
| ATOM | 10648 | O | ALA | 1589 | 28.148 | −7.055 | 14.116 | 1.00 | 17.78 |
| ATOM | 10649 | N | TYR | 1590 | 30.024 | −6.023 | 13.421 | 1.00 | 14.09 |
| ATOM | 10650 | CA | TYR | 1590 | 30.911 | −7.130 | 13.775 | 1.00 | 15.98 |
| ATOM | 10651 | CB | TYR | 1590 | 31.164 | −8.010 | 12.543 | 1.00 | 16.40 |
| ATOM | 10652 | CG | TYR | 1590 | 31.512 | −7.250 | 11.277 | 1.00 | 15.39 |
| ATOM | 10653 | CD1 | TYR | 1590 | 32.816 | −7.232 | 10.783 | 1.00 | 13.60 |
| ATOM | 10654 | CE1 | TYR | 1590 | 33.127 | −6.558 | 9.596 | 1.00 | 15.26 |
| ATOM | 10655 | CD2 | TYR | 1590 | 30.524 | −6.571 | 10.559 | 1.00 | 14.41 |
| ATOM | 10656 | CE2 | TYR | 1590 | 30.818 | −5.895 | 9.378 | 1.00 | 14.95 |
| ATOM | 10657 | CZ | TYR | 1590 | 32.118 | −5.895 | 8.901 | 1.00 | 14.53 |
| ATOM | 10658 | OH | TYR | 1590 | 32.380 | −5.247 | 7.718 | 1.00 | 14.98 |
| ATOM | 10659 | C | TYR | 1590 | 32.225 | −6.601 | 14.334 | 1.00 | 16.91 |
| ATOM | 10660 | O | TYR | 1590 | 33.303 | −7.077 | 13.980 | 1.00 | 17.15 |
| ATOM | 10661 | N | ALA | 1591 | 32.109 | −5.626 | 15.235 | 1.00 | 16.90 |
| ATOM | 10662 | CA | ALA | 1591 | 33.257 | −4.973 | 15.863 | 1.00 | 18.12 |
| ATOM | 10663 | CB | ALA | 1591 | 32.790 | −3.734 | 16.617 | 1.00 | 16.82 |
| ATOM | 10664 | C | ALA | 1591 | 34.012 | −5.909 | 16.798 | 1.00 | 17.70 |
| ATOM | 10665 | O | ALA | 1591 | 35.212 | −5.759 | 17.005 | 1.00 | 16.35 |
| ATOM | 10666 | N | THR | 1592 | 33.289 | −6.868 | 17.366 | 1.00 | 17.84 |
| ATOM | 10667 | CA | THR | 1592 | 33.871 | −7.863 | 18.254 | 1.00 | 17.19 |
| ATOM | 10668 | CB | THR | 1592 | 33.583 | −7.570 | 19.739 | 1.00 | 17.53 |
| ATOM | 10669 | OG1 | THR | 1592 | 32.176 | −7.682 | 19.991 | 1.00 | 16.88 |
| ATOM | 10670 | CG2 | THR | 1592 | 34.059 | −6.169 | 20.112 | 1.00 | 16.17 |
| ATOM | 10671 | C | THR | 1592 | 33.208 | −9.184 | 17.900 | 1.00 | 15.81 |
| ATOM | 10672 | O | THR | 1592 | 32.114 | −9.201 | 17.329 | 1.00 | 13.62 |
| ATOM | 10673 | N | PRO | 1593 | 33.860 | −10.310 | 18.223 | 1.00 | 16.07 |
| ATOM | 10674 | CD | PRO | 1593 | 35.254 | −10.465 | 18.675 | 1.00 | 16.83 |
| ATOM | 10675 | CA | PRO | 1593 | 33.260 | −11.607 | 17.903 | 1.00 | 16.05 |
| ATOM | 10676 | CB | PRO | 1593 | 34.278 | −12.599 | 18.459 | 1.00 | 17.78 |
| ATOM | 10677 | CG | PRO | 1593 | 35.587 | −11.877 | 18.233 | 1.00 | 16.98 |
| ATOM | 10678 | C | PRO | 1593 | 31.874 | −11.769 | 18.531 | 1.00 | 16.88 |
| ATOM | 10679 | O | PRO | 1593 | 30.934 | −12.190 | 17.858 | 1.00 | 15.90 |
| ATOM | 10680 | N | GLU | 1594 | 31.746 | −11.417 | 19.810 | 1.00 | 17.16 |
| ATOM | 10681 | CA | GLU | 1594 | 30.467 | −11.548 | 20.507 | 1.00 | 17.81 |
| ATOM | 10682 | CB | GLU | 1594 | 30.606 | −11.095 | 21.966 | 1.00 | 21.81 |
| ATOM | 10683 | CG | GLU | 1594 | 29.392 | −11.407 | 22.830 | 1.00 | 29.07 |
| ATOM | 10684 | CD | GLU | 1594 | 29.657 | −11.163 | 24.308 | 1.00 | 34.97 |
| ATOM | 10685 | OE1 | GLU | 1594 | 30.087 | −10.036 | 24.649 | 1.00 | 37.17 |
| ATOM | 10686 | OE2 | GLU | 1594 | 29.429 | −12.091 | 25.124 | 1.00 | 37.45 |
| ATOM | 10687 | C | GLU | 1594 | 29.369 | −10.755 | 19.817 | 1.00 | 16.20 |
| ATOM | 10688 | O | GLU | 1594 | 28.233 | −11.210 | 19.721 | 1.00 | 14.75 |
| ATOM | 10689 | N | GLN | 1595 | 29.708 | −9.568 | 19.326 | 1.00 | 16.15 |
| ATOM | 10690 | CA | GLN | 1595 | 28.715 | −8.749 | 18.645 | 1.00 | 16.61 |
| ATOM | 10691 | CB | GLN | 1595 | 29.209 | −7.319 | 18.489 | 1.00 | 19.07 |
| ATOM | 10692 | CG | GLN | 1595 | 29.345 | −6.591 | 19.814 | 1.00 | 25.36 |
| ATOM | 10693 | CD | GLN | 1595 | 29.618 | −5.113 | 19.634 | 1.00 | 28.24 |
| ATOM | 10694 | OE1 | GLN | 1595 | 28.854 | −4.411 | 18.974 | 1.00 | 30.74 |
| ATOM | 10695 | NE2 | GLN | 1595 | 30.709 | −4.629 | 20.228 | 1.00 | 31.60 |
| ATOM | 10696 | C | GLN | 1595 | 28.401 | −9.344 | 17.282 | 1.00 | 13.90 |
| ATOM | 10697 | O | GLN | 1595 | 27.261 | −9.290 | 16.826 | 1.00 | 12.90 |
| ATOM | 10698 | N | ALA | 1596 | 29.410 | −9.918 | 16.636 | 1.00 | 12.16 |
| ATOM | 10699 | CA | ALA | 1596 | 29.184 | −10.530 | 15.339 | 1.00 | 12.33 |
| ATOM | 10700 | CB | ALA | 1596 | 30.494 | −10.993 | 14.730 | 1.00 | 11.02 |
| ATOM | 10701 | C | ALA | 1596 | 28.212 | −11.702 | 15.484 | 1.00 | 12.04 |
| ATOM | 10702 | O | ALA | 1596 | 27.345 | −11.897 | 14.632 | 1.00 | 11.82 |
| ATOM | 10703 | N | PHE | 1597 | 28.340 | −12.470 | 16.566 | 1.00 | 13.20 |
| ATOM | 10704 | CA | PHE | 1597 | 27.460 | −13.630 | 16.787 | 1.00 | 12.23 |
| ATOM | 10705 | CB | PHE | 1597 | 27.823 | −14.392 | 18.077 | 1.00 | 11.34 |
| ATOM | 10706 | CG | PHE | 1597 | 29.267 | −14.829 | 18.165 | 1.00 | 12.72 |
| ATOM | 10707 | CD1 | PHE | 1597 | 29.996 | −15.142 | 17.018 | 1.00 | 14.75 |
| ATOM | 10708 | CD2 | PHE | 1597 | 29.881 | −14.963 | 19.406 | 1.00 | 13.59 |
| ATOM | 10709 | CE1 | PHE | 1597 | 31.323 | −15.586 | 17.107 | 1.00 | 15.56 |
| ATOM | 10710 | CE2 | PHE | 1597 | 31.204 | −15.407 | 19.513 | 1.00 | 13.42 |
| ATOM | 10711 | CZ | PHE | 1597 | 31.921 | −15.718 | 18.354 | 1.00 | 15.98 |
| ATOM | 10712 | C | PHE | 1597 | 26.005 | −13.205 | 16.912 | 1.00 | 12.63 |
| ATOM | 10713 | O | PHE | 1597 | 25.114 | −13.834 | 16.346 | 1.00 | 12.34 |
| ATOM | 10714 | N | GLU | 1598 | 25.778 | −12.134 | 17.667 | 1.00 | 12.52 |
| ATOM | 10715 | CA | GLU | 1598 | 24.434 | −11.641 | 17.892 | 1.00 | 13.41 |
| ATOM | 10716 | CB | GLU | 1598 | 24.456 | −10.541 | 18.955 | 1.00 | 16.36 |
| ATOM | 10717 | CG | GLU | 1598 | 23.081 | −10.001 | 19.313 | 1.00 | 23.47 |
| ATOM | 10718 | CD | GLU | 1598 | 22.273 | −10.950 | 20.189 | 1.00 | 28.97 |
| ATOM | 10719 | OE1 | GLU | 1598 | 22.481 | −12.188 | 20.109 | 1.00 | 31.03 |
| ATOM | 10720 | OE2 | GLU | 1598 | 21.414 | −10.452 | 20.953 | 1.00 | 32.01 |
| ATOM | 10721 | C | GLU | 1598 | 23.783 | −11.113 | 16.615 | 1.00 | 12.74 |
| ATOM | 10722 | O | GLU | 1598 | 22.634 | −11.425 | 16.321 | 1.00 | 11.28 |
| ATOM | 10723 | N | ASN | 1599 | 24.518 | −10.316 | 15.852 | 1.00 | 12.02 |
| ATOM | 10724 | CA | ASN | 1599 | 23.953 | −9.758 | 14.640 | 1.00 | 11.59 |
| ATOM | 10725 | CB | ASN | 1599 | 24.740 | −8.513 | 14.228 | 1.00 | 12.97 |
| ATOM | 10726 | CG | ASN | 1599 | 24.671 | −7.422 | 15.295 | 1.00 | 13.26 |
| ATOM | 10727 | OD1 | ASN | 1599 | 23.617 | −7.218 | 15.908 | 1.00 | 13.34 |
| ATOM | 10728 | ND2 | ASN | 1599 | 25.774 | −6.712 | 15.510 | 1.00 | 13.73 |
| ATOM | 10729 | C | ASN | 1599 | 23.865 | −10.771 | 13.507 | 1.00 | 11.88 |
| ATOM | 10730 | O | ASN | 1599 | 22.961 | −10.696 | 12.677 | 1.00 | 11.95 |
| ATOM | 10731 | N | ALA | 1600 | 24.790 | −11.724 | 13.471 | 1.00 | 9.53 |
| ATOM | 10732 | CA | ALA | 1600 | 24.732 | −12.754 | 12.440 | 1.00 | 11.81 |
| ATOM | 10733 | CB | ALA | 1600 | 25.994 | −13.610 | 12.462 | 1.00 | 11.15 |
| ATOM | 10734 | C | ALA | 1600 | 23.503 | −13.615 | 12.742 | 1.00 | 11.76 |
| ATOM | 10735 | O | ALA | 1600 | 22.763 | −13.990 | 11.838 | 1.00 | 12.58 |
| ATOM | 10736 | N | ALA | 1601 | 23.283 | −13.922 | 14.019 | 1.00 | 11.05 |
| ATOM | 10737 | CA | ALA | 1601 | 22.142 | −14.746 | 14.391 | 1.00 | 11.07 |
| ATOM | 10738 | CB | ALA | 1601 | 22.183 | −15.080 | 15.889 | 1.00 | 12.88 |
| ATOM | 10739 | C | ALA | 1601 | 20.830 | −14.051 | 14.038 | 1.00 | 11.65 |
| ATOM | 10740 | O | ALA | 1601 | 19.868 | −14.700 | 13.624 | 1.00 | 12.48 |
| ATOM | 10741 | N | THR | 1602 | 20.782 | −12.735 | 14.198 | 1.00 | 10.09 |
| ATOM | 10742 | CA | THR | 1602 | 19.565 | −11.980 | 13.884 | 1.00 | 10.55 |
| ATOM | 10743 | CB | THR | 1602 | 19.718 | −10.486 | 14.224 | 1.00 | 10.24 |
| ATOM | 10744 | OG1 | THR | 1602 | 19.875 | −10.342 | 15.638 | 1.00 | 12.17 |
| ATOM | 10745 | CG2 | THR | 1602 | 18.491 | −9.703 | 13.773 | 1.00 | 11.96 |
| ATOM | 10746 | C | THR | 1602 | 19.211 | −12.089 | 12.408 | 1.00 | 11.71 |
| ATOM | 10747 | O | THR | 1602 | 18.060 | −12.356 | 12.052 | 1.00 | 10.21 |
| ATOM | 10748 | N | VAL | 1603 | 20.193 | −11.884 | 11.538 | 1.00 | 8.99 |
| ATOM | 10749 | CA | VAL | 1603 | 19.897 | −11.956 | 10.120 | 1.00 | 10.74 |
| ATOM | 10750 | CB | VAL | 1603 | 20.997 | −11.276 | 9.280 | 1.00 | 12.65 |
| ATOM | 10751 | CG1 | VAL | 1603 | 20.519 | −11.129 | 7.858 | 1.00 | 19.78 |
| ATOM | 10752 | CG2 | VAL | 1603 | 21.309 | −9.885 | 9.854 | 1.00 | 13.29 |
| ATOM | 10753 | C | VAL | 1603 | 19.679 | −13.401 | 9.675 | 1.00 | 10.88 |
| ATOM | 10754 | O | VAL | 1603 | 18.931 | −13.651 | 8.733 | 1.00 | 11.56 |
| ATOM | 10755 | N | MET | 1604 | 20.322 | −14.354 | 10.352 | 1.00 | 10.50 |
| ATOM | 10756 | CA | MET | 1604 | 20.118 | −15.768 | 10.024 | 1.00 | 11.08 |
| ATOM | 10757 | CB | MET | 1604 | 21.082 | −16.672 | 10.806 | 1.00 | 15.41 |
| ATOM | 10758 | CG | MET | 1604 | 22.562 | −16.538 | 10.462 | 1.00 | 18.50 |
| ATOM | 10759 | SD | MET | 1604 | 22.958 | −17.034 | 8.795 | 1.00 | 27.30 |
| ATOM | 10760 | CE | MET | 1604 | 22.871 | −18.884 | 8.916 | 1.00 | 21.09 |
| ATOM | 10761 | C | MET | 1604 | 18.677 | −16.169 | 10.383 | 1.00 | 10.81 |
| ATOM | 10762 | O | MET | 1604 | 17.984 | −16.794 | 9.584 | 1.00 | 13.07 |
| ATOM | 10763 | N | ARG | 1605 | 18.222 | −15.811 | 11.584 | 1.00 | 11.02 |
| ATOM | 10764 | CA | ARG | 1605 | 16.861 | −16.158 | 11.992 | 1.00 | 12.52 |
| ATOM | 10765 | CB | ARG | 1605 | 16.582 | −15.724 | 13.444 | 1.00 | 13.71 |
| ATOM | 10766 | CG | ARG | 1605 | 17.431 | −16.445 | 14.496 | 1.00 | 13.88 |
| ATOM | 10767 | CD | ARG | 1605 | 16.798 | −16.395 | 15.889 | 1.00 | 14.01 |
| ATOM | 10768 | NE | ARG | 1605 | 17.771 | −16.750 | 16.919 | 1.00 | 18.79 |
| ATOM | 10769 | CZ | ARG | 1605 | 18.649 | −15.898 | 17.433 | 1.00 | 18.25 |
| ATOM | 10770 | NH1 | ARG | 1605 | 18.670 | −14.636 | 17.025 | 1.00 | 18.55 |
| ATOM | 10771 | NH2 | ARG | 1605 | 19.521 | −16.314 | 18.333 | 1.00 | 18.62 |
| ATOM | 10772 | C | ARG | 1605 | 15.814 | −15.534 | 11.070 | 1.00 | 12.91 |
| ATOM | 10773 | O | ARG | 1605 | 14.724 | −16.090 | 10.900 | 1.00 | 12.21 |
| ATOM | 10774 | N | ALA | 1606 | 16.141 | −14.386 | 10.481 | 1.00 | 11.94 |
| ATOM | 10775 | CA | ALA | 1606 | 15.223 | −13.704 | 9.575 | 1.00 | 12.22 |
| ATOM | 10776 | CB | ALA | 1606 | 15.632 | −12.232 | 9.409 | 1.00 | 11.74 |
| ATOM | 10777 | C | ALA | 1606 | 15.136 | −14.374 | 8.199 | 1.00 | 13.18 |
| ATOM | 10778 | O | ALA | 1606 | 14.274 | −14.021 | 7.392 | 1.00 | 14.17 |
| ATOM | 10779 | N | GLY | 1607 | 16.023 | −15.331 | 7.929 | 1.00 | 12.39 |
| ATOM | 10780 | CA | GLY | 1607 | 15.980 | −16.029 | 6.653 | 1.00 | 11.87 |
| ATOM | 10781 | C | GLY | 1607 | 17.288 | −16.273 | 5.907 | 1.00 | 11.83 |
| ATOM | 10782 | O | GLY | 1607 | 17.316 | −17.063 | 4.960 | 1.00 | 15.36 |
| ATOM | 10783 | N | ALA | 1608 | 18.368 | −15.612 | 6.316 | 1.00 | 11.14 |
| ATOM | 10784 | CA | ALA | 1608 | 19.668 | −15.767 | 5.651 | 1.00 | 11.81 |
| ATOM | 10785 | CB | ALA | 1608 | 20.621 | −14.689 | 6.146 | 1.00 | 13.69 |
| ATOM | 10786 | C | ALA | 1608 | 20.294 | −17.151 | 5.870 | 1.00 | 13.84 |
| ATOM | 10787 | O | ALA | 1608 | 20.013 | −17.799 | 6.870 | 1.00 | 11.53 |
| ATOM | 10788 | N | ASN | 1609 | 21.135 | −17.602 | 4.934 | 1.00 | 13.34 |
| ATOM | 10789 | CA | ASN | 1609 | 21.814 | −18.895 | 5.077 | 1.00 | 13.10 |
| ATOM | 10790 | CB | ASN | 1609 | 21.736 | −19.734 | 3.797 | 1.00 | 14.21 |
| ATOM | 10791 | CG | ASN | 1609 | 20.321 | −20.082 | 3.407 | 1.00 | 15.93 |
| ATOM | 10792 | OD1 | ASN | 1609 | 19.606 | −20.775 | 4.137 | 1.00 | 18.98 |
| ATOM | 10793 | ND2 | ASN | 1609 | 19.910 | −19.610 | 2.250 | 1.00 | 12.95 |
| ATOM | 10794 | C | ASN | 1609 | 23.293 | −18.648 | 5.344 | 1.00 | 14.73 |
| ATOM | 10795 | O | ASN | 1609 | 24.021 | −19.533 | 5.794 | 1.00 | 15.75 |
| ATOM | 10796 | N | MET | 1610 | 23.732 | −17.435 | 5.049 | 1.00 | 13.00 |
| ATOM | 10797 | CA | MET | 1610 | 25.132 | −17.083 | 5.197 | 1.00 | 13.92 |
| ATOM | 10798 | CB | MET | 1610 | 25.864 | −17.514 | 3.925 | 1.00 | 14.99 |
| ATOM | 10799 | CG | MET | 1610 | 27.293 | −17.047 | 3.789 | 1.00 | 18.34 |
| ATOM | 10800 | SD | MET | 1610 | 27.934 | −17.568 | 2.175 | 1.00 | 19.59 |
| ATOM | 10801 | CE | MET | 1610 | 28.973 | −18.934 | 2.662 | 1.00 | 21.61 |
| ATOM | 10802 | C | MET | 1610 | 25.270 | −15.586 | 5.421 | 1.00 | 13.22 |
| ATOM | 10803 | O | MET | 1610 | 24.400 | −14.808 | 5.028 | 1.00 | 12.57 |
| ATOM | 10804 | N | VAL | 1611 | 26.366 | −15.185 | 6.053 | 1.00 | 13.16 |
| ATOM | 10805 | CA | VAL | 1611 | 26.603 | −13.782 | 6.336 | 1.00 | 14.59 |
| ATOM | 10806 | CB | VAL | 1611 | 26.750 | −13.580 | 7.859 | 1.00 | 18.07 |
| ATOM | 10807 | CG1 | VAL | 1611 | 27.320 | −12.237 | 8.152 | 1.00 | 23.20 |
| ATOM | 10808 | CG2 | VAL | 1611 | 25.389 | −13.741 | 8.535 | 1.00 | 17.45 |
| ATOM | 10809 | C | VAL | 1611 | 27.853 | −13.289 | 5.621 | 1.00 | 15.11 |
| ATOM | 10810 | O | VAL | 1611 | 28.844 | −14.016 | 5.518 | 1.00 | 13.59 |
| ATOM | 10811 | N | LYS | 1612 | 27.797 | −12.061 | 5.108 | 1.00 | 13.93 |
| ATOM | 10812 | CA | LYS | 1612 | 28.942 | −11.471 | 4.420 | 1.00 | 13.65 |
| ATOM | 10813 | CB | LYS | 1612 | 28.541 | −10.980 | 3.023 | 1.00 | 16.26 |
| ATOM | 10814 | CG | LYS | 1612 | 29.718 | −10.435 | 2.207 | 1.00 | 16.17 |
| ATOM | 10815 | CD | LYS | 1612 | 29.419 | −10.402 | 0.703 | 1.00 | 19.74 |
| ATOM | 10816 | CE | LYS | 1612 | 28.447 | −9.294 | 0.338 | 1.00 | 20.53 |
| ATOM | 10817 | NZ | LYS | 1612 | 29.054 | −7.938 | 0.547 | 1.00 | 18.43 |
| ATOM | 10818 | C | LYS | 1612 | 29.476 | −10.307 | 5.239 | 1.00 | 14.73 |
| ATOM | 10819 | O | LYS | 1612 | 28.709 | −9.454 | 5.680 | 1.00 | 15.46 |
| ATOM | 10820 | N | ILE | 1613 | 30.792 | −10.280 | 5.439 | 1.00 | 15.79 |
| ATOM | 10821 | CA | ILE | 1613 | 31.436 | −9.215 | 6.210 | 1.00 | 17.39 |
| ATOM | 10822 | CB | ILE | 1613 | 31.868 | −9.699 | 7.623 | 1.00 | 17.61 |
| ATOM | 10823 | CG2 | ILE | 1613 | 30.642 | −9.920 | 8.507 | 1.00 | 18.44 |
| ATOM | 10824 | CG1 | ILE | 1613 | 32.706 | −10.972 | 7.502 | 1.00 | 18.71 |
| ATOM | 10825 | CD1 | ILE | 1613 | 33.102 | −11.573 | 8.835 | 1.00 | 21.00 |
| ATOM | 10826 | C | ILE | 1613 | 32.674 | −8.698 | 5.487 | 1.00 | 17.52 |
| ATOM | 10827 | O | ILE | 1613 | 33.363 | −9.453 | 4.805 | 1.00 | 17.34 |
| ATOM | 10828 | N | GLU | 1614 | 32.948 | −7.410 | 5.658 | 1.00 | 18.44 |
| ATOM | 10829 | CA | GLU | 1614 | 34.077 | −6.756 | 5.014 | 1.00 | 22.04 |
| ATOM | 10830 | CB | GLU | 1614 | 33.699 | −5.319 | 4.650 | 1.00 | 21.72 |
| ATOM | 10831 | CG | GLU | 1614 | 32.556 | −5.213 | 3.663 | 1.00 | 24.57 |
| ATOM | 10832 | CD | GLU | 1614 | 32.210 | −3.773 | 3.316 | 1.00 | 25.01 |
| ATOM | 10833 | OE1 | GLU | 1614 | 33.048 | −2.879 | 3.551 | 1.00 | 25.95 |
| ATOM | 10834 | OE2 | GLU | 1614 | 31.103 | −3.535 | 2.793 | 1.00 | 26.44 |
| ATOM | 10835 | C | GLU | 1614 | 35.330 | −6.730 | 5.879 | 1.00 | 22.81 |
| ATOM | 10836 | O | GLU | 1614 | 35.270 | −6.387 | 7.059 | 1.00 | 23.72 |
| ATOM | 10837 | N | GLY | 1615 | 36.469 | −7.082 | 5.292 | 1.00 | 24.76 |
| ATOM | 10838 | CA | GLY | 1615 | 37.706 | −7.063 | 6.052 | 1.00 | 26.31 |
| ATOM | 10839 | C | GLY | 1615 | 38.657 | −8.191 | 5.724 | 1.00 | 25.48 |
| ATOM | 10840 | O | GLY | 1615 | 38.264 | −9.195 | 5.132 | 1.00 | 26.13 |
| ATOM | 10841 | N | GLY | 1616 | 39.916 | −8.024 | 6.117 | 1.00 | 26.28 |
| ATOM | 10842 | CA | GLY | 1616 | 40.917 | −9.043 | 5.855 | 1.00 | 25.88 |
| ATOM | 10843 | C | GLY | 1616 | 41.395 | −9.771 | 7.101 | 1.00 | 26.83 |
| ATOM | 10844 | O | GLY | 1616 | 40.592 | −10.313 | 7.862 | 1.00 | 26.50 |
| ATOM | 10845 | N | GLU | 1617 | 42.712 | −9.780 | 7.293 | 1.00 | 23.60 |
| ATOM | 10846 | CA | GLU | 1617 | 43.369 | −10.427 | 8.427 | 1.00 | 25.78 |
| ATOM | 10847 | CB | GLU | 1617 | 44.811 | −9.923 | 8.554 | 1.00 | 28.43 |
| ATOM | 10848 | CG | GLU | 1617 | 45.856 | −10.775 | 7.868 | 1.00 | 35.94 |
| ATOM | 10849 | CD | GLU | 1617 | 46.136 | −12.074 | 8.604 | 1.00 | 39.30 |
| ATOM | 10850 | OE1 | GLU | 1617 | 45.199 | −12.883 | 8.770 | 1.00 | 39.90 |
| ATOM | 10851 | OE2 | GLU | 1617 | 47.299 | −12.283 | 9.015 | 1.00 | 39.51 |
| ATOM | 10852 | C | GLU | 1617 | 42.718 | −10.294 | 9.800 | 1.00 | 23.15 |
| ATOM | 10853 | O | GLU | 1617 | 42.527 | −11.289 | 10.499 | 1.00 | 24.44 |
| ATOM | 10854 | N | TRP | 1618 | 42.404 | −9.069 | 10.202 | 1.00 | 22.84 |
| ATOM | 10855 | CA | TRP | 1618 | 41.830 | −8.855 | 11.518 | 1.00 | 20.46 |
| ATOM | 10856 | CB | TRP | 1618 | 41.607 | −7.364 | 11.776 | 1.00 | 21.59 |
| ATOM | 10857 | CG | TRP | 1618 | 40.461 | −6.766 | 11.040 | 1.00 | 19.95 |
| ATOM | 10858 | CD2 | TRP | 1618 | 39.152 | −6.510 | 11.563 | 1.00 | 19.68 |
| ATOM | 10859 | CE2 | TRP | 1618 | 38.392 | −5.939 | 10.521 | 1.00 | 17.75 |
| ATOM | 10860 | CE3 | TRP | 1618 | 38.544 | −6.723 | 12.809 | 1.00 | 18.42 |
| ATOM | 10861 | CD1 | TRP | 1618 | 40.446 | −6.348 | 9.745 | 1.00 | 20.31 |
| ATOM | 10862 | NE1 | TRP | 1618 | 39.205 | −5.845 | 9.423 | 1.00 | 19.55 |
| ATOM | 10863 | CZ2 | TRP | 1618 | 37.054 | −5.557 | 10.690 | 1.00 | 17.13 |
| ATOM | 10864 | CZ3 | TRP | 1618 | 37.214 | −6.345 | 12.977 | 1.00 | 18.71 |
| ATOM | 10865 | CH2 | TRP | 1618 | 36.482 | −5.771 | 11.915 | 1.00 | 19.07 |
| ATOM | 10866 | C | TRP | 1618 | 40.544 | −9.619 | 11.787 | 1.00 | 20.59 |
| ATOM | 10867 | O | TRP | 1618 | 40.136 | −9.746 | 12.938 | 1.00 | 18.49 |
| ATOM | 10868 | N | LEU | 1619 | 39.917 | −10.144 | 10.737 | 1.00 | 21.43 |
| ATOM | 10869 | CA | LEU | 1619 | 38.668 | −10.886 | 10.893 | 1.00 | 20.49 |
| ATOM | 10870 | CB | LEU | 1619 | 37.751 | −10.622 | 9.696 | 1.00 | 20.97 |
| ATOM | 10871 | CG | LEU | 1619 | 37.106 | −9.238 | 9.620 | 1.00 | 21.93 |
| ATOM | 10872 | CD1 | LEU | 1619 | 36.316 | −9.117 | 8.338 | 1.00 | 22.24 |
| ATOM | 10873 | CD2 | LEU | 1619 | 36.200 | −9.039 | 10.833 | 1.00 | 23.75 |
| ATOM | 10874 | C | LEU | 1619 | 38.817 | −12.395 | 11.067 | 1.00 | 19.39 |
| ATOM | 10875 | O | LEU | 1619 | 37.821 | −13.094 | 11.238 | 1.00 | 16.11 |
| ATOM | 10876 | N | VAL | 1620 | 40.047 | −12.899 | 11.034 | 1.00 | 18.75 |
| ATOM | 10877 | CA | VAL | 1620 | 40.284 | −14.334 | 11.174 | 1.00 | 18.47 |
| ATOM | 10878 | CB | VAL | 1620 | 41.798 | −14.645 | 11.309 | 1.00 | 20.23 |
| ATOM | 10879 | CG1 | VAL | 1620 | 42.007 | −16.107 | 11.660 | 1.00 | 20.87 |
| ATOM | 10880 | CG2 | VAL | 1620 | 42.505 | −14.331 | 10.003 | 1.00 | 22.98 |
| ATOM | 10881 | C | VAL | 1620 | 39.553 | −14.972 | 12.349 | 1.00 | 18.83 |
| ATOM | 10882 | O | VAL | 1620 | 38.847 | −15.965 | 12.173 | 1.00 | 18.24 |
| ATOM | 10883 | N | GLU | 1621 | 39.731 | −14.411 | 13.543 | 1.00 | 18.17 |
| ATOM | 10884 | CA | GLU | 1621 | 39.088 | −14.946 | 14.741 | 1.00 | 18.85 |
| ATOM | 10885 | CB | GLU | 1621 | 39.479 | −14.109 | 15.969 | 1.00 | 18.97 |
| ATOM | 10886 | CG | GLU | 1621 | 38.815 | −14.561 | 17.256 | 1.00 | 22.60 |
| ATOM | 10887 | CD | GLU | 1621 | 39.265 | −13.760 | 18.471 | 1.00 | 25.26 |
| ATOM | 10888 | OE1 | GLU | 1621 | 39.153 | −12.515 | 18.453 | 1.00 | 24.17 |
| ATOM | 10889 | OE2 | GLU | 1621 | 39.730 | −14.383 | 19.446 | 1.00 | 28.44 |
| ATOM | 10890 | C | GLU | 1621 | 37.571 | −14.962 | 14.599 | 1.00 | 16.87 |
| ATOM | 10891 | O | GLU | 1621 | 36.915 | −15.951 | 14.909 | 1.00 | 16.16 |
| ATOM | 10892 | N | THR | 1622 | 37.023 | −13.855 | 14.118 | 1.00 | 17.23 |
| ATOM | 10893 | CA | THR | 1622 | 35.583 | −13.729 | 13.938 | 1.00 | 15.12 |
| ATOM | 10894 | CB | THR | 1622 | 35.238 | −12.319 | 13.420 | 1.00 | 16.38 |
| ATOM | 10895 | OG1 | THR | 1622 | 35.730 | −11.345 | 14.356 | 1.00 | 17.00 |
| ATOM | 10896 | CG2 | THR | 1622 | 33.727 | −12.140 | 13.272 | 1.00 | 14.48 |
| ATOM | 10897 | C | THR | 1622 | 35.048 | −14.797 | 12.985 | 1.00 | 15.13 |
| ATOM | 10898 | O | THR | 1622 | 34.064 | −15.464 | 13.293 | 1.00 | 13.37 |
| ATOM | 10899 | N | VAL | 1623 | 35.696 | −14.963 | 11.832 | 1.00 | 13.92 |
| ATOM | 10900 | CA | VAL | 1623 | 35.258 | −15.969 | 10.862 | 1.00 | 14.72 |
| ATOM | 10901 | CB | VAL | 1623 | 36.102 | −15.907 | 9.569 | 1.00 | 14.99 |
| ATOM | 10902 | CG1 | VAL | 1623 | 35.676 | −17.017 | 8.602 | 1.00 | 16.56 |
| ATOM | 10903 | CG2 | VAL | 1623 | 35.936 | −14.548 | 8.921 | 1.00 | 14.47 |
| ATOM | 10904 | C | VAL | 1623 | 35.345 | −17.382 | 11.452 | 1.00 | 15.17 |
| ATOM | 10905 | O | VAL | 1623 | 34.424 | −18.186 | 11.308 | 1.00 | 13.99 |
| ATOM | 10906 | N | GLN | 1624 | 36.456 | −17.680 | 12.118 | 1.00 | 14.83 |
| ATOM | 10907 | CA | GLN | 1624 | 36.641 | −18.992 | 12.729 | 1.00 | 17.43 |
| ATOM | 10908 | CB | GLN | 1624 | 38.012 | −19.073 | 13.415 | 1.00 | 20.41 |
| ATOM | 10909 | CG | GLN | 1624 | 39.205 | −18.806 | 12.499 | 1.00 | 28.20 |
| ATOM | 10910 | CD | GLN | 1624 | 40.540 | −18.963 | 13.210 | 1.00 | 31.26 |
| ATOM | 10911 | OE1 | GLN | 1624 | 40.789 | −18.329 | 14.238 | 1.00 | 33.11 |
| ATOM | 10912 | NE2 | GLN | 1624 | 41.408 | −19.809 | 12.661 | 1.00 | 32.88 |
| ATOM | 10913 | C | GLN | 1624 | 35.544 | −19.296 | 13.755 | 1.00 | 15.38 |
| ATOM | 10914 | O | GLN | 1624 | 34.966 | −20.384 | 13.757 | 1.00 | 15.85 |
| ATOM | 10915 | N | MET | 1625 | 35.266 | −18.334 | 14.632 | 1.00 | 16.26 |
| ATOM | 10916 | CA | MET | 1625 | 34.249 | −18.528 | 15.664 | 1.00 | 14.73 |
| ATOM | 10917 | CB | MET | 1625 | 34.398 | −17.466 | 16.758 | 1.00 | 15.61 |
| ATOM | 10918 | CG | MET | 1625 | 35.637 | −17.671 | 17.623 | 1.00 | 18.12 |
| ATOM | 10919 | SD | MET | 1625 | 35.862 | −16.357 | 18.837 | 1.00 | 20.56 |
| ATOM | 10920 | CE | MET | 1625 | 34.733 | −16.876 | 20.128 | 1.00 | 24.48 |
| ATOM | 10921 | C | MET | 1625 | 32.832 | −18.534 | 15.104 | 1.00 | 15.23 |
| ATOM | 10922 | O | MET | 1625 | 31.976 | −19.288 | 15.571 | 1.00 | 15.05 |
| ATOM | 10923 | N | LEU | 1626 | 32.572 | −17.702 | 14.101 | 1.00 | 16.96 |
| ATOM | 10924 | CA | LEU | 1626 | 31.240 | −17.694 | 13.504 | 1.00 | 15.20 |
| ATOM | 10925 | CB | LEU | 1626 | 31.139 | −16.617 | 12.416 | 1.00 | 15.28 |
| ATOM | 10926 | CG | LEU | 1626 | 30.816 | −15.209 | 12.916 | 1.00 | 12.23 |
| ATOM | 10927 | CD1 | LEU | 1626 | 30.993 | −14.196 | 11.790 | 1.00 | 12.86 |
| ATOM | 10928 | CD2 | LEU | 1626 | 29.398 | −15.188 | 13.455 | 1.00 | 11.50 |
| ATOM | 10929 | C | LEU | 1626 | 30.947 | −19.065 | 12.900 | 1.00 | 16.67 |
| ATOM | 10930 | O | LEU | 1626 | 29.905 | −19.679 | 13.161 | 1.00 | 16.01 |
| ATOM | 10931 | N | THR | 1627 | 31.887 | −19.548 | 12.098 | 1.00 | 16.42 |
| ATOM | 10932 | CA | THR | 1627 | 31.743 | −20.833 | 11.423 | 1.00 | 19.44 |
| ATOM | 10933 | CB | THR | 1627 | 33.000 | −21.152 | 10.592 | 1.00 | 20.87 |
| ATOM | 10934 | OG1 | THR | 1627 | 33.259 | −20.061 | 9.698 | 1.00 | 22.73 |
| ATOM | 10935 | CG2 | THR | 1627 | 32.790 | −22.424 | 9.770 | 1.00 | 26.46 |
| ATOM | 10936 | C | THR | 1627 | 31.481 | −21.983 | 12.382 | 1.00 | 18.98 |
| ATOM | 10937 | O | THR | 1627 | 30.573 | −22.782 | 12.168 | 1.00 | 20.35 |
| ATOM | 10938 | N | GLU | 1628 | 32.267 | −22.072 | 13.446 | 1.00 | 20.15 |
| ATOM | 10939 | CA | GLU | 1628 | 32.053 | −23.160 | 14.378 | 1.00 | 21.96 |
| ATOM | 10940 | CB | GLU | 1628 | 33.257 | −23.296 | 15.313 | 1.00 | 25.32 |
| ATOM | 10941 | CG | GLU | 1628 | 33.231 | −22.418 | 16.525 | 1.00 | 28.20 |
| ATOM | 10942 | CD | GLU | 1628 | 34.458 | −22.615 | 17.394 | 1.00 | 27.97 |
| ATOM | 10943 | OE1 | GLU | 1628 | 34.989 | −23.748 | 17.440 | 1.00 | 27.80 |
| ATOM | 10944 | OE2 | GLU | 1628 | 34.884 | −21.638 | 18.039 | 1.00 | 28.14 |
| ATOM | 10945 | C | GLU | 1628 | 30.737 | −22.990 | 15.155 | 1.00 | 21.32 |
| ATOM | 10946 | O | GLU | 1628 | 30.278 | −23.924 | 15.807 | 1.00 | 21.40 |
| ATOM | 10947 | N | ARG | 1629 | 30.127 | −21.806 | 15.073 | 1.00 | 18.90 |
| ATOM | 10948 | CA | ARG | 1629 | 28.850 | −21.555 | 15.739 | 1.00 | 18.94 |
| ATOM | 10949 | CB | ARG | 1629 | 28.877 | −20.193 | 16.455 | 1.00 | 19.15 |
| ATOM | 10950 | CG | ARG | 1629 | 29.636 | −20.242 | 17.778 | 1.00 | 15.98 |
| ATOM | 10951 | CD | ARG | 1629 | 30.159 | −18.882 | 18.246 | 1.00 | 12.70 |
| ATOM | 10952 | NE | ARG | 1629 | 30.954 | −19.047 | 19.460 | 1.00 | 13.17 |
| ATOM | 10953 | CZ | ARG | 1629 | 32.154 | −19.616 | 19.498 | 1.00 | 12.49 |
| ATOM | 10954 | NH1 | ARG | 1629 | 32.719 | −20.068 | 18.382 | 1.00 | 14.22 |
| ATOM | 10955 | NH2 | ARG | 1629 | 32.778 | −19.774 | 20.658 | 1.00 | 15.37 |
| ATOM | 10956 | C | ARG | 1629 | 27.659 | −21.633 | 14.768 | 1.00 | 19.42 |
| ATOM | 10957 | O | ARG | 1629 | 26.610 | −21.029 | 15.006 | 1.00 | 21.03 |
| ATOM | 10958 | N | ALA | 1630 | 27.838 | −22.373 | 13.671 | 1.00 | 19.16 |
| ATOM | 10959 | CA | ALA | 1630 | 26.792 | −22.591 | 12.663 | 1.00 | 17.42 |
| ATOM | 10960 | CB | ALA | 1630 | 25.493 | −23.018 | 13.346 | 1.00 | 15.89 |
| ATOM | 10961 | C | ALA | 1630 | 26.503 | −21.444 | 11.698 | 1.00 | 16.92 |
| ATOM | 10962 | O | ALA | 1630 | 25.460 | −21.433 | 11.052 | 1.00 | 15.55 |
| ATOM | 10963 | N | VAL | 1631 | 27.412 | −20.484 | 11.590 | 1.00 | 16.60 |
| ATOM | 10964 | CA | VAL | 1631 | 27.194 | −19.369 | 10.685 | 1.00 | 15.88 |
| ATOM | 10965 | CB | VAL | 1631 | 27.282 | −18.019 | 11.436 | 1.00 | 17.04 |
| ATOM | 10966 | CG1 | VAL | 1631 | 26.996 | −16.863 | 10.477 | 1.00 | 15.90 |
| ATOM | 10967 | CG2 | VAL | 1631 | 26.280 | −17.994 | 12.590 | 1.00 | 18.38 |
| ATOM | 10968 | C | VAL | 1631 | 28.200 | −19.357 | 9.534 | 1.00 | 15.71 |
| ATOM | 10969 | O | VAL | 1631 | 29.375 | −19.046 | 9.728 | 1.00 | 17.57 |
| ATOM | 10970 | N | PRO | 1632 | 27.760 | −19.725 | 8.318 | 1.00 | 16.10 |
| ATOM | 10971 | CD | PRO | 1632 | 26.468 | −20.292 | 7.901 | 1.00 | 14.50 |
| ATOM | 10972 | CA | PRO | 1632 | 28.710 | −19.706 | 7.200 | 1.00 | 15.24 |
| ATOM | 10973 | CB | PRO | 1632 | 27.961 | −20.438 | 6.083 | 1.00 | 15.98 |
| ATOM | 10974 | CG | PRO | 1632 | 26.552 | −20.188 | 6.393 | 1.00 | 20.78 |
| ATOM | 10975 | C | PRO | 1632 | 29.025 | −18.255 | 6.857 | 1.00 | 16.57 |
| ATOM | 10976 | O | PRO | 1632 | 28.156 | −17.380 | 6.933 | 1.00 | 15.01 |
| ATOM | 10977 | N | VAL | 1633 | 30.269 | −18.003 | 6.478 | 1.00 | 14.68 |
| ATOM | 10978 | CA | VAL | 1633 | 30.703 | −16.654 | 6.174 | 1.00 | 14.60 |
| ATOM | 10979 | CB | VAL | 1633 | 31.808 | −16.213 | 7.157 | 1.00 | 16.39 |
| ATOM | 10980 | CG1 | VAL | 1633 | 32.210 | −14.773 | 6.874 | 1.00 | 16.35 |
| ATOM | 10981 | CG2 | VAL | 1633 | 31.320 | −16.371 | 8.593 | 1.00 | 15.41 |
| ATOM | 10982 | C | VAL | 1633 | 31.237 | −16.462 | 4.770 | 1.00 | 14.92 |
| ATOM | 10983 | O | VAL | 1633 | 31.952 | −17.311 | 4.234 | 1.00 | 14.86 |
| ATOM | 10984 | N | CYS | 1634 | 30.875 | −15.332 | 4.180 | 1.00 | 13.16 |
| ATOM | 10985 | CA | CYS | 1634 | 31.352 | −14.972 | 2.855 | 1.00 | 15.22 |
| ATOM | 10986 | CB | CYS | 1634 | 30.186 | −14.545 | 1.955 | 1.00 | 14.14 |
| ATOM | 10987 | SG | CYS | 1634 | 30.713 | −13.922 | 0.336 | 1.00 | 17.65 |
| ATOM | 10988 | C | CYS | 1634 | 32.277 | −13.794 | 3.132 | 1.00 | 14.44 |
| ATOM | 10989 | O | CYS | 1634 | 31.929 | −12.892 | 3.887 | 1.00 | 15.27 |
| ATOM | 10990 | N | GLY | 1635 | 33.471 | −13.816 | 2.558 | 1.00 | 15.52 |
| ATOM | 10991 | CA | GLY | 1635 | 34.400 | −12.723 | 2.779 | 1.00 | 14.14 |
| ATOM | 10992 | C | GLY | 1635 | 34.162 | −11.615 | 1.771 | 1.00 | 15.47 |
| ATOM | 10993 | O | GLY | 1635 | 33.352 | −11.776 | 0.866 | 1.00 | 16.16 |
| ATOM | 10994 | N | HIS | 1636 | 34.867 | −10.498 | 1.918 | 1.00 | 16.26 |
| ATOM | 10995 | CA | HIS | 1636 | 34.709 | −9.364 | 1.009 | 1.00 | 18.95 |
| ATOM | 10996 | CB | HIS | 1636 | 33.468 | −8.553 | 1.413 | 1.00 | 19.60 |
| ATOM | 10997 | CG | HIS | 1636 | 33.099 | −7.460 | 0.456 | 1.00 | 20.41 |
| ATOM | 10998 | CD2 | HIS | 1636 | 33.752 | −6.934 | −0.607 | 1.00 | 20.13 |
| ATOM | 10999 | ND1 | HIS | 1636 | 31.917 | −6.757 | 0.561 | 1.00 | 20.81 |
| ATOM | 11000 | CE1 | HIS | 1636 | 31.858 | −5.847 | −0.393 | 1.00 | 20.55 |
| ATOM | 11001 | NE2 | HIS | 1636 | 32.960 | −5.933 | −1.116 | 1.00 | 20.46 |
| ATOM | 11002 | C | HIS | 1636 | 35.959 | −8.491 | 1.073 | 1.00 | 21.06 |
| ATOM | 11003 | O | HIS | 1636 | 36.171 | −7.769 | 2.050 | 1.00 | 23.03 |
| ATOM | 11004 | N | LEU | 1637 | 36.783 | −8.571 | 0.030 | 1.00 | 22.97 |
| ATOM | 11005 | CA | LEU | 1637 | 38.028 | −7.804 | −0.050 | 1.00 | 24.89 |
| ATOM | 11006 | CB | LEU | 1637 | 39.227 | −8.755 | −0.153 | 1.00 | 24.90 |
| ATOM | 11007 | CG | LEU | 1637 | 39.479 | −9.659 | 1.057 | 1.00 | 26.13 |
| ATOM | 11008 | CD1 | LEU | 1637 | 40.618 | −10.621 | 0.773 | 1.00 | 24.93 |
| ATOM | 11009 | CD2 | LEU | 1637 | 39.804 | −8.798 | 2.267 | 1.00 | 26.07 |
| ATOM | 11010 | C | LEU | 1637 | 38.026 | −6.854 | −1.243 | 1.00 | 26.65 |
| ATOM | 11011 | O | LEU | 1637 | 37.199 | −6.978 | −2.147 | 1.00 | 24.18 |
| ATOM | 11012 | N | GLY | 1638 | 38.962 | −5.909 | −1.234 | 1.00 | 28.25 |
| ATOM | 11013 | CA | GLY | 1638 | 39.063 | −4.941 | −2.309 | 1.00 | 28.56 |
| ATOM | 11014 | C | GLY | 1638 | 38.402 | −3.634 | −1.928 | 1.00 | 29.01 |
| ATOM | 11015 | O | GLY | 1638 | 38.664 | −3.081 | −0.858 | 1.00 | 29.30 |
| ATOM | 11016 | N | LEU | 1639 | 37.537 | −3.141 | −2.805 | 1.00 | 28.17 |
| ATOM | 11017 | CA | LEU | 1639 | 36.824 | −1.898 | −2.566 | 1.00 | 27.72 |
| ATOM | 11018 | CB | LEU | 1639 | 36.311 | −1.348 | −3.899 | 1.00 | 29.18 |
| ATOM | 11019 | CG | LEU | 1639 | 36.026 | 0.153 | −3.987 | 1.00 | 30.77 |
| ATOM | 11020 | CD1 | LEU | 1639 | 35.697 | 0.527 | −5.427 | 1.00 | 31.76 |
| ATOM | 11021 | CD2 | LEU | 1639 | 34.890 | 0.522 | −3.066 | 1.00 | 30.98 |
| ATOM | 11022 | C | LEU | 1639 | 35.657 | −2.190 | −1.620 | 1.00 | 28.43 |
| ATOM | 11023 | O | LEU | 1639 | 34.575 | −2.581 | −2.063 | 1.00 | 26.50 |
| ATOM | 11024 | N | THR | 1640 | 35.890 | −2.013 | −0.320 | 1.00 | 28.20 |
| ATOM | 11025 | CA | THR | 1640 | 34.866 | −2.259 | 0.698 | 1.00 | 28.91 |
| ATOM | 11026 | CB | THR | 1640 | 35.482 | −2.866 | 1.970 | 1.00 | 29.17 |
| ATOM | 11027 | OG1 | THR | 1640 | 36.517 | −2.006 | 2.462 | 1.00 | 30.62 |
| ATOM | 11028 | CG2 | THR | 1640 | 36.068 | −4.236 | 1.670 | 1.00 | 30.17 |
| ATOM | 11029 | C | THR | 1640 | 34.171 | −0.951 | 1.057 | 1.00 | 28.17 |
| ATOM | 11030 | O | THR | 1640 | 34.667 | −0.177 | 1.874 | 1.00 | 29.15 |
| ATOM | 11031 | N | PRO | 1641 | 32.991 | −0.706 | 0.463 | 1.00 | 27.49 |
| ATOM | 11032 | CD | PRO | 1641 | 32.251 | −1.695 | −0.339 | 1.00 | 27.96 |
| ATOM | 11033 | CA | PRO | 1641 | 32.189 | 0.502 | 0.674 | 1.00 | 26.18 |
| ATOM | 11034 | CB | PRO | 1641 | 30.897 | 0.202 | −0.092 | 1.00 | 26.35 |
| ATOM | 11035 | CG | PRO | 1641 | 30.832 | −1.290 | −0.098 | 1.00 | 29.35 |
| ATOM | 11036 | C | PRO | 1641 | 31.952 | 0.977 | 2.104 | 1.00 | 25.02 |
| ATOM | 11037 | O | PRO | 1641 | 31.763 | 2.174 | 2.332 | 1.00 | 24.64 |
| ATOM | 11038 | N | GLN | 1642 | 31.968 | 0.064 | 3.070 | 1.00 | 23.18 |
| ATOM | 11039 | CA | GLN | 1642 | 31.768 | 0.475 | 4.459 | 1.00 | 23.68 |
| ATOM | 11040 | CB | GLN | 1642 | 31.661 | −0.751 | 5.369 | 1.00 | 24.34 |
| ATOM | 11041 | CG | GLN | 1642 | 30.249 | −1.307 | 5.471 | 1.00 | 22.71 |
| ATOM | 11042 | CD | GLN | 1642 | 30.192 | −2.633 | 6.204 | 1.00 | 25.04 |
| ATOM | 11043 | OE1 | GLN | 1642 | 31.017 | −2.910 | 7.075 | 1.00 | 23.03 |
| ATOM | 11044 | NE2 | GLN | 1642 | 29.204 | −3.456 | 5.865 | 1.00 | 23.95 |
| ATOM | 11045 | C | GLN | 1642 | 32.922 | 1.367 | 4.911 | 1.00 | 24.23 |
| ATOM | 11046 | O | GLN | 1642 | 32.781 | 2.160 | 5.842 | 1.00 | 24.32 |
| ATOM | 11047 | N | SER | 1643 | 34.061 | 1.235 | 4.237 | 1.00 | 23.66 |
| ATOM | 11048 | CA | SER | 1643 | 35.246 | 2.028 | 4.559 | 1.00 | 24.17 |
| ATOM | 11049 | CB | SER | 1643 | 36.478 | 1.126 | 4.596 | 1.00 | 23.24 |
| ATOM | 11050 | OG | SER | 1643 | 36.342 | 0.116 | 5.570 | 1.00 | 21.44 |
| ATOM | 11051 | C | SER | 1643 | 35.474 | 3.157 | 3.556 | 1.00 | 25.48 |
| ATOM | 11052 | O | SER | 1643 | 36.602 | 3.618 | 3.368 | 1.00 | 24.46 |
| ATOM | 11053 | N | VAL | 1644 | 34.400 | 3.598 | 2.908 | 1.00 | 26.06 |
| ATOM | 11054 | CA | VAL | 1644 | 34.502 | 4.671 | 1.924 | 1.00 | 26.14 |
| ATOM | 11055 | CB | VAL | 1644 | 33.109 | 5.080 | 1.402 | 1.00 | 25.49 |
| ATOM | 11056 | CG1 | VAL | 1644 | 32.258 | 5.623 | 2.541 | 1.00 | 26.03 |
| ATOM | 11057 | CG2 | VAL | 1644 | 33.250 | 6.110 | 0.293 | 1.00 | 25.29 |
| ATOM | 11058 | C | VAL | 1644 | 35.209 | 5.905 | 2.501 | 1.00 | 27.56 |
| ATOM | 11059 | O | VAL | 1644 | 36.063 | 6.500 | 1.842 | 1.00 | 27.45 |
| ATOM | 11060 | N | ASN | 1645 | 34.866 | 6.282 | 3.732 | 1.00 | 27.89 |
| ATOM | 11061 | CA | ASN | 1645 | 35.480 | 7.452 | 4.359 | 1.00 | 28.29 |
| ATOM | 11062 | CB | ASN | 1645 | 34.779 | 7.776 | 5.682 | 1.00 | 27.21 |
| ATOM | 11063 | CG | ASN | 1645 | 33.331 | 8.190 | 5.486 | 1.00 | 25.74 |
| ATOM | 11064 | OD1 | ASN | 1645 | 33.045 | 9.268 | 4.967 | 1.00 | 22.31 |
| ATOM | 11065 | ND2 | ASN | 1645 | 32.408 | 7.321 | 5.888 | 1.00 | 25.99 |
| ATOM | 11066 | C | ASN | 1645 | 36.971 | 7.241 | 4.599 | 1.00 | 29.33 |
| ATOM | 11067 | O | ASN | 1645 | 37.735 | 8.205 | 4.676 | 1.00 | 29.64 |
| ATOM | 11068 | N | ILE | 1646 | 37.373 | 5.978 | 4.715 | 1.00 | 30.28 |
| ATOM | 11069 | CA | ILE | 1646 | 38.773 | 5.626 | 4.931 | 1.00 | 31.32 |
| ATOM | 11070 | CB | ILE | 1646 | 38.929 | 4.153 | 5.396 | 1.00 | 31.95 |
| ATOM | 11071 | CG2 | ILE | 1646 | 40.399 | 3.752 | 5.377 | 1.00 | 31.20 |
| ATOM | 11072 | CG1 | ILE | 1646 | 38.355 | 3.977 | 6.806 | 1.00 | 31.77 |
| ATOM | 11073 | CD1 | ILE | 1646 | 39.071 | 4.789 | 7.862 | 1.00 | 31.01 |
| ATOM | 11074 | C | ILE | 1646 | 39.562 | 5.809 | 3.636 | 1.00 | 33.51 |
| ATOM | 11075 | O | ILE | 1646 | 40.655 | 6.377 | 3.644 | 1.00 | 34.28 |
| ATOM | 11076 | N | PHE | 1647 | 39.006 | 5.324 | 2.526 | 1.00 | 34.55 |
| ATOM | 11077 | CA | PHE | 1647 | 39.664 | 5.439 | 1.225 | 1.00 | 35.75 |
| ATOM | 11078 | CB | PHE | 1647 | 39.083 | 4.429 | 0.227 | 1.00 | 36.28 |
| ATOM | 11079 | CG | PHE | 1647 | 39.051 | 3.014 | 0.734 | 1.00 | 37.21 |
| ATOM | 11080 | CD1 | PHE | 1647 | 40.183 | 2.435 | 1.300 | 1.00 | 37.15 |
| ATOM | 11081 | CD2 | PHE | 1647 | 37.885 | 2.257 | 0.638 | 1.00 | 36.77 |
| ATOM | 11082 | CE1 | PHE | 1647 | 40.154 | 1.120 | 1.763 | 1.00 | 38.58 |
| ATOM | 11083 | CE2 | PHE | 1647 | 37.845 | 0.943 | 1.096 | 1.00 | 36.69 |
| ATOM | 11084 | CZ | PHE | 1647 | 38.981 | 0.373 | 1.661 | 1.00 | 38.01 |
| ATOM | 11085 | C | PHE | 1647 | 39.512 | 6.840 | 0.643 | 1.00 | 36.39 |
| ATOM | 11086 | O | PHE | 1647 | 40.280 | 7.243 | −0.233 | 1.00 | 36.05 |
| ATOM | 11087 | N | GLY | 1648 | 38.518 | 7.577 | 1.130 | 1.00 | 36.62 |
| ATOM | 11088 | CA | GLY | 1648 | 38.279 | 8.919 | 0.630 | 1.00 | 38.30 |
| ATOM | 11089 | C | GLY | 1648 | 37.421 | 8.878 | −0.622 | 1.00 | 40.02 |
| ATOM | 11090 | O | GLY | 1648 | 37.406 | 9.822 | −1.413 | 1.00 | 40.07 |
| ATOM | 11091 | N | GLY | 1649 | 36.703 | 7.773 | −0.796 | 1.00 | 40.99 |
| ATOM | 11092 | CA | GLY | 1649 | 35.846 | 7.609 | −1.956 | 1.00 | 42.04 |
| ATOM | 11093 | C | GLY | 1649 | 35.898 | 6.184 | −2.477 | 1.00 | 42.47 |
| ATOM | 11094 | O | GLY | 1649 | 36.534 | 5.324 | −1.876 | 1.00 | 41.84 |
| ATOM | 11095 | N | TYR | 1650 | 35.229 | 5.927 | −3.596 | 1.00 | 44.57 |
| ATOM | 11096 | CA | TYR | 1650 | 35.224 | 4.592 | −4.178 | 1.00 | 45.61 |
| ATOM | 11097 | CB | TYR | 1650 | 33.839 | 4.258 | −4.735 | 1.00 | 46.67 |
| ATOM | 11098 | CG | TYR | 1650 | 32.731 | 4.386 | −3.713 | 1.00 | 48.02 |
| ATOM | 11099 | CD1 | TYR | 1650 | 32.142 | 5.623 | −3.448 | 1.00 | 47.87 |
| ATOM | 11100 | CE1 | TYR | 1650 | 31.142 | 5.753 | −2.487 | 1.00 | 48.46 |
| ATOM | 11101 | CD2 | TYR | 1650 | 32.291 | 3.275 | −2.988 | 1.00 | 48.35 |
| ATOM | 11102 | CE2 | TYR | 1650 | 31.291 | 3.394 | −2.021 | 1.00 | 48.79 |
| ATOM | 11103 | CZ | TYR | 1650 | 30.722 | 4.637 | −1.777 | 1.00 | 48.77 |
| ATOM | 11104 | OH | TYR | 1650 | 29.735 | 4.767 | −0.827 | 1.00 | 48.30 |
| ATOM | 11105 | C | TYR | 1650 | 36.272 | 4.498 | −5.280 | 1.00 | 46.09 |
| ATOM | 11106 | O | TYR | 1650 | 35.975 | 4.697 | −6.458 | 1.00 | 45.93 |
| ATOM | 11107 | N | LYS | 1651 | 37.503 | 4.195 | −4.881 | 1.00 | 46.34 |
| ATOM | 11108 | CA | LYS | 1651 | 38.614 | 4.077 | −5.815 | 1.00 | 46.77 |
| ATOM | 11109 | CB | LYS | 1651 | 39.805 | 4.890 | −5.306 | 1.00 | 47.81 |
| ATOM | 11110 | CG | LYS | 1651 | 39.467 | 6.325 | −4.935 | 1.00 | 49.04 |
| ATOM | 11111 | CD | LYS | 1651 | 40.686 | 7.053 | −4.385 | 1.00 | 50.24 |
| ATOM | 11112 | CE | LYS | 1651 | 40.343 | 8.473 | −3.962 | 1.00 | 51.45 |
| ATOM | 11113 | NZ | LYS | 1651 | 41.543 | 9.208 | −3.468 | 1.00 | 53.01 |
| ATOM | 11114 | C | LYS | 1651 | 39.026 | 2.618 | −5.982 | 1.00 | 46.53 |
| ATOM | 11115 | O | LYS | 1651 | 38.815 | 1.797 | −5.087 | 1.00 | 46.02 |
| ATOM | 11116 | N | VAL | 1652 | 39.614 | 2.300 | −7.131 | 1.00 | 46.03 |
| ATOM | 11117 | CA | VAL | 1652 | 40.063 | 0.941 | −7.409 | 1.00 | 45.48 |
| ATOM | 11118 | CB | VAL | 1652 | 40.715 | 0.838 | −8.803 | 1.00 | 45.22 |
| ATOM | 11119 | CG1 | VAL | 1652 | 41.216 | −0.577 | −9.039 | 1.00 | 45.09 |
| ATOM | 11120 | CG2 | VAL | 1652 | 39.708 | 1.228 | −9.874 | 1.00 | 44.78 |
| ATOM | 11121 | C | VAL | 1652 | 41.080 | 0.510 | −6.360 | 1.00 | 45.62 |
| ATOM | 11122 | O | VAL | 1652 | 41.910 | 1.307 | −5.921 | 1.00 | 44.90 |
| ATOM | 11123 | N | GLN | 1653 | 41.012 | −0.755 | −5.963 | 1.00 | 45.29 |
| ATOM | 11124 | CA | GLN | 1653 | 41.917 | −1.286 | −4.955 | 1.00 | 45.81 |
| ATOM | 11125 | CB | GLN | 1653 | 41.108 | −1.795 | −3.758 | 1.00 | 46.31 |
| ATOM | 11126 | CG | GLN | 1653 | 41.733 | −1.510 | −2.405 | 1.00 | 47.72 |
| ATOM | 11127 | CD | GLN | 1653 | 41.812 | −0.025 | −2.097 | 1.00 | 47.56 |
| ATOM | 11128 | OE1 | GLN | 1653 | 40.805 | 0.683 | −2.129 | 1.00 | 47.95 |
| ATOM | 11129 | NE2 | GLN | 1653 | 43.011 | 0.451 | −1.790 | 1.00 | 49.46 |
| ATOM | 11130 | C | GLN | 1653 | 42.745 | −2.422 | −5.549 | 1.00 | 45.65 |
| ATOM | 11131 | O | GLN | 1653 | 42.340 | −3.046 | −6.529 | 1.00 | 45.37 |
| ATOM | 11132 | N | GLY | 1654 | 43.907 | −2.686 | −4.957 | 1.00 | 45.93 |
| ATOM | 11133 | CA | GLY | 1654 | 44.758 | −3.753 | −5.456 | 1.00 | 46.97 |
| ATOM | 11134 | C | GLY | 1654 | 45.912 | −3.277 | −6.324 | 1.00 | 47.53 |
| ATOM | 11135 | O | GLY | 1654 | 46.917 | −3.974 | −6.457 | 1.00 | 46.50 |
| ATOM | 11136 | N | ARG | 1655 | 45.763 | −2.096 | −6.920 | 1.00 | 48.61 |
| ATOM | 11137 | CA | ARG | 1655 | 46.793 | −1.511 | −7.778 | 1.00 | 49.79 |
| ATOM | 11138 | CB | ARG | 1655 | 46.421 | −0.067 | −8.141 | 1.00 | 50.69 |
| ATOM | 11139 | CG | ARG | 1655 | 45.152 | 0.090 | −8.980 | 1.00 | 51.67 |
| ATOM | 11140 | CD | ARG | 1655 | 45.449 | 0.007 | −10.469 | 1.00 | 52.58 |
| ATOM | 11141 | NE | ARG | 1655 | 44.245 | 0.096 | −11.297 | 1.00 | 52.70 |
| ATOM | 11142 | CZ | ARG | 1655 | 43.421 | 1.141 | −11.329 | 1.00 | 52.20 |
| ATOM | 11143 | NH1 | ARG | 1655 | 43.658 | 2.205 | −10.574 | 1.00 | 52.02 |
| ATOM | 11144 | NH2 | ARG | 1655 | 42.360 | 1.124 | −12.125 | 1.00 | 51.43 |
| ATOM | 11145 | C | ARG | 1655 | 48.146 | −1.512 | −7.071 | 1.00 | 50.62 |
| ATOM | 11146 | O | ARG | 1655 | 48.328 | −0.826 | −6.065 | 1.00 | 50.32 |
| ATOM | 11147 | N | GLY | 1656 | 49.093 | −2.282 | −7.597 | 1.00 | 51.16 |
| ATOM | 11148 | CA | GLY | 1656 | 50.410 | −2.339 | −6.989 | 1.00 | 52.70 |
| ATOM | 11149 | C | GLY | 1656 | 50.786 | −3.730 | −6.522 | 1.00 | 53.14 |
| ATOM | 11150 | O | GLY | 1656 | 49.959 | −4.640 | −6.528 | 1.00 | 53.30 |
| ATOM | 11151 | N | ASP | 1657 | 52.041 | −3.898 | −6.119 | 1.00 | 53.65 |
| ATOM | 11152 | CA | ASP | 1657 | 52.523 | −5.190 | −5.649 | 1.00 | 53.80 |
| ATOM | 11153 | CB | ASP | 1657 | 54.034 | −5.308 | −5.870 | 1.00 | 55.47 |
| ATOM | 11154 | CG | ASP | 1657 | 54.406 | −5.377 | −7.340 | 1.00 | 56.62 |
| ATOM | 11155 | OD1 | ASP | 1657 | 53.962 | −6.327 | −8.022 | 1.00 | 56.60 |
| ATOM | 11156 | OD2 | ASP | 1657 | 55.144 | −4.484 | −7.812 | 1.00 | 57.58 |
| ATOM | 11157 | C | ASP | 1657 | 52.204 | −5.392 | −4.173 | 1.00 | 53.09 |
| ATOM | 11158 | O | ASP | 1657 | 51.620 | −6.404 | −3.793 | 1.00 | 52.84 |
| ATOM | 11159 | N | GLU | 1658 | 52.586 | −4.424 | −3.347 | 1.00 | 52.53 |
| ATOM | 11160 | CA | GLU | 1658 | 52.340 | −4.507 | −1.912 | 1.00 | 52.30 |
| ATOM | 11161 | CB | GLU | 1658 | 52.820 | −3.232 | −1.214 | 1.00 | 53.11 |
| ATOM | 11162 | CG | GLU | 1658 | 52.733 | −3.294 | 0.306 | 1.00 | 54.62 |
| ATOM | 11163 | CD | GLU | 1658 | 53.180 | −2.007 | 0.973 | 1.00 | 55.75 |
| ATOM | 11164 | OE1 | GLU | 1658 | 54.319 | −1.562 | 0.708 | 1.00 | 56.22 |
| ATOM | 11165 | OE2 | GLU | 1658 | 52.393 | −1.443 | 1.768 | 1.00 | 55.93 |
| ATOM | 11166 | C | GLU | 1658 | 50.859 | −4.722 | −1.616 | 1.00 | 51.14 |
| ATOM | 11167 | O | GLU | 1658 | 50.491 | −5.640 | −0.882 | 1.00 | 50.90 |
| ATOM | 11168 | N | ALA | 1659 | 50.015 | −3.870 | −2.190 | 1.00 | 49.84 |
| ATOM | 11169 | CA | ALA | 1659 | 48.573 | −3.969 | −1.990 | 1.00 | 47.95 |
| ATOM | 11170 | CB | ALA | 1659 | 47.869 | −2.804 | −2.675 | 1.00 | 48.45 |
| ATOM | 11171 | C | ALA | 1659 | 48.048 | −5.293 | −2.535 | 1.00 | 46.33 |
| ATOM | 11172 | O | ALA | 1659 | 47.280 | −5.987 | −1.869 | 1.00 | 46.58 |
| ATOM | 11173 | N | GLY | 1660 | 48.470 | −5.635 | −3.748 | 1.00 | 44.15 |
| ATOM | 11174 | CA | GLY | 1660 | 48.035 | −6.874 | −4.363 | 1.00 | 41.45 |
| ATOM | 11175 | C | GLY | 1660 | 48.372 | −8.095 | −3.529 | 1.00 | 40.33 |
| ATOM | 11176 | O | GLY | 1660 | 47.507 | −8.925 | −3.260 | 1.00 | 38.67 |
| ATOM | 11177 | N | ASP | 1661 | 49.630 | −8.209 | −3.114 | 1.00 | 39.99 |
| ATOM | 11178 | CA | ASP | 1661 | 50.055 | −9.348 | −2.312 | 1.00 | 39.26 |
| ATOM | 11179 | CB | ASP | 1661 | 51.563 | −9.293 | −2.058 | 1.00 | 42.10 |
| ATOM | 11180 | CG | ASP | 1661 | 52.368 | −9.258 | −3.340 | 1.00 | 42.83 |
| ATOM | 11181 | OD1 | ASP | 1661 | 52.150 | −10.129 | −4.208 | 1.00 | 42.87 |
| ATOM | 11182 | OD2 | ASP | 1661 | 53.223 | −8.360 | −3.477 | 1.00 | 45.93 |
| ATOM | 11183 | C | ASP | 1661 | 49.317 | −9.386 | −0.982 | 1.00 | 38.30 |
| ATOM | 11184 | O | ASP | 1661 | 49.029 | −10.460 | −0.455 | 1.00 | 36.90 |
| ATOM | 11185 | N | GLN | 1662 | 49.014 | −8.211 | −0.442 | 1.00 | 37.31 |
| ATOM | 11186 | CA | GLN | 1662 | 48.306 | −8.124 | 0.829 | 1.00 | 37.91 |
| ATOM | 11187 | CB | GLN | 1662 | 48.299 | −6.678 | 1.336 | 1.00 | 39.32 |
| ATOM | 11188 | CG | GLN | 1662 | 47.557 | −6.500 | 2.647 | 1.00 | 42.13 |
| ATOM | 11189 | CD | GLN | 1662 | 47.934 | −7.555 | 3.671 | 1.00 | 43.63 |
| ATOM | 11190 | OE1 | GLN | 1662 | 49.108 | −7.733 | 3.993 | 1.00 | 44.57 |
| ATOM | 11191 | NE2 | GLN | 1662 | 46.937 | −8.261 | 4.186 | 1.00 | 45.08 |
| ATOM | 11192 | C | GLN | 1662 | 46.872 | −8.631 | 0.694 | 1.00 | 36.59 |
| ATOM | 11193 | O | GLN | 1662 | 46.316 | −9.209 | 1.632 | 1.00 | 35.25 |
| ATOM | 11194 | N | LEU | 1663 | 46.278 | −8.408 | −0.474 | 1.00 | 36.15 |
| ATOM | 11195 | CA | LEU | 1863 | 44.911 | −8.851 | −0.730 | 1.00 | 35.64 |
| ATOM | 11196 | CB | LEU | 1663 | 44.359 | −8.190 | −1.999 | 1.00 | 37.34 |
| ATOM | 11197 | CG | LEU | 1663 | 44.061 | −6.686 | −1.919 | 1.00 | 39.82 |
| ATOM | 11198 | CD1 | LEU | 1663 | 43.626 | −6.168 | −3.283 | 1.00 | 40.42 |
| ATOM | 11199 | CD2 | LEU | 1663 | 42.971 | −6.434 | −0.884 | 1.00 | 40.78 |
| ATOM | 11200 | C | LEU | 1663 | 44.880 | −10.367 | −0.875 | 1.00 | 33.87 |
| ATOM | 11201 | O | LEU | 1663 | 43.997 | −11.030 | −0.333 | 1.00 | 33.09 |
| ATOM | 11202 | N | LEU | 1664 | 45.850 | −10.912 | −1.604 | 1.00 | 31.69 |
| ATOM | 11203 | CA | LEU | 1664 | 45.933 | −12.353 | −1.808 | 1.00 | 30.95 |
| ATOM | 11204 | CB | LEU | 1664 | 47.102 | −12.690 | −2.736 | 1.00 | 34.05 |
| ATOM | 11205 | CG | LEU | 1664 | 46.949 | −13.905 | −3.655 | 1.00 | 35.86 |
| ATOM | 11206 | CD1 | LEU | 1664 | 48.242 | −14.089 | −4.444 | 1.00 | 37.45 |
| ATOM | 11207 | CD2 | LEU | 1664 | 46.639 | −15.155 | −2.853 | 1.00 | 36.48 |
| ATOM | 11208 | C | LEU | 1664 | 46.141 | −13.026 | −0.454 | 1.00 | 29.09 |
| ATOM | 11209 | O | LEU | 1664 | 45.610 | −14.107 | −0.190 | 1.00 | 28.40 |
| ATOM | 11210 | N | SER | 1665 | 46.921 | −12.375 | 0.403 | 1.00 | 26.84 |
| ATOM | 11211 | CA | SER | 1665 | 47.192 | −12.897 | 1.733 | 1.00 | 25.84 |
| ATOM | 11212 | CB | SER | 1665 | 48.222 | −12.017 | 2.447 | 1.00 | 26.63 |
| ATOM | 11213 | OG | SER | 1665 | 48.565 | −12.558 | 3.713 | 1.00 | 28.52 |
| ATOM | 11214 | C | SER | 1665 | 45.892 | −12.931 | 2.533 | 1.00 | 24.57 |
| ATOM | 11215 | O | SER | 1665 | 45.552 | −13.949 | 3.132 | 1.00 | 23.68 |
| ATOM | 11216 | N | ASP | 1666 | 45.166 | −11.815 | 2.532 | 1.00 | 24.47 |
| ATOM | 11217 | CA | ASP | 1666 | 43.901 | −11.738 | 3.258 | 1.00 | 25.90 |
| ATOM | 11218 | CB | ASP | 1666 | 43.296 | −10.328 | 3.170 | 1.00 | 25.16 |
| ATOM | 11219 | CG | ASP | 1666 | 44.089 | −9.296 | 3.957 | 1.00 | 27.13 |
| ATOM | 11220 | OD1 | ASP | 1666 | 44.664 | −9.652 | 5.009 | 1.00 | 26.45 |
| ATOM | 11221 | OD2 | ASP | 1666 | 44.130 | −8.117 | 3.534 | 1.00 | 27.04 |
| ATOM | 11222 | C | ASP | 1666 | 42.913 | −12.759 | 2.714 | 1.00 | 25.78 |
| ATOM | 11223 | O | ASP | 1666 | 42.168 | −13.382 | 3.473 | 1.00 | 27.18 |
| ATOM | 11224 | N | ALA | 1667 | 42.910 | −12.936 | 1.397 | 1.00 | 25.37 |
| ATOM | 11225 | CA | ALA | 1667 | 42.013 | −13.893 | 0.765 | 1.00 | 24.67 |
| ATOM | 11226 | CB | ALA | 1667 | 42.187 | −13.849 | −0.754 | 1.00 | 24.78 |
| ATOM | 11227 | C | ALA | 1667 | 42.297 | −15.294 | 1.287 | 1.00 | 24.19 |
| ATOM | 11228 | O | ALA | 1667 | 41.382 | −16.026 | 1.673 | 1.00 | 22.33 |
| ATOM | 11229 | N | LEU | 1668 | 43.570 | −15.673 | 1.303 | 1.00 | 23.07 |
| ATOM | 11230 | CA | LEU | 1668 | 43.951 | −16.996 | 1.792 | 1.00 | 22.47 |
| ATOM | 11231 | CB | LEU | 1668 | 45.434 | −17.256 | 1.506 | 1.00 | 23.09 |
| ATOM | 11232 | CG | LEU | 1668 | 45.767 | −17.598 | 0.047 | 1.00 | 23.46 |
| ATOM | 11233 | CD1 | LEU | 1668 | 47.259 | −17.456 | −0.188 | 1.00 | 24.35 |
| ATOM | 11234 | CD2 | LEU | 1668 | 45.297 | −19.006 | −0.278 | 1.00 | 23.63 |
| ATOM | 11235 | C | LEU | 1668 | 43.676 | −17.140 | 3.288 | 1.00 | 22.33 |
| ATOM | 11236 | O | LEU | 1668 | 43.296 | −18.211 | 3.751 | 1.00 | 22.16 |
| ATOM | 11237 | N | ALA | 1669 | 43.877 | −16.059 | 4.038 | 1.00 | 22.43 |
| ATOM | 11238 | CA | ALA | 1669 | 43.633 | −16.083 | 5.479 | 1.00 | 22.18 |
| ATOM | 11239 | CB | ALA | 1669 | 44.086 | −14.782 | 6.111 | 1.00 | 23.50 |
| ATOM | 11240 | C | ALA | 1669 | 42.153 | −16.328 | 5.775 | 1.00 | 21.87 |
| ATOM | 11241 | O | ALA | 1669 | 41.809 | −17.177 | 6.600 | 1.00 | 22.22 |
| ATOM | 11242 | N | LEU | 1670 | 41.284 | −15.585 | 5.098 | 1.00 | 20.26 |
| ATOM | 11243 | CA | LEU | 1670 | 39.846 | −15.730 | 5.288 | 1.00 | 18.56 |
| ATOM | 11244 | CB | LEU | 1670 | 39.085 | −14.737 | 4.401 | 1.00 | 19.24 |
| ATOM | 11245 | CG | LEU | 1670 | 39.265 | −13.260 | 4.755 | 1.00 | 18.45 |
| ATOM | 11246 | CD1 | LEU | 1670 | 38.703 | −12.375 | 3.662 | 1.00 | 19.75 |
| ATOM | 11247 | CD2 | LEU | 1670 | 38.569 | −12.973 | 6.086 | 1.00 | 19.43 |
| ATOM | 11248 | C | LEU | 1670 | 39.405 | −17.146 | 4.962 | 1.00 | 19.70 |
| ATOM | 11249 | O | LEU | 1670 | 38.571 | −17.714 | 5.659 | 1.00 | 18.51 |
| ATOM | 11250 | N | GLU | 1671 | 39.963 | −17.720 | 3.901 | 1.00 | 18.99 |
| ATOM | 11251 | CA | GLU | 1671 | 39.603 | −19.079 | 3.518 | 1.00 | 19.04 |
| ATOM | 11252 | CB | GLU | 1671 | 40.303 | −19.474 | 2.215 | 1.00 | 19.59 |
| ATOM | 11253 | CG | GLU | 1671 | 40.072 | −20.929 | 1.816 | 1.00 | 22.01 |
| ATOM | 11254 | CD | GLU | 1671 | 40.790 | −21.305 | 0.530 | 1.00 | 24.31 |
| ATOM | 11255 | OE1 | GLU | 1671 | 42.015 | −21.096 | 0.458 | 1.00 | 23.91 |
| ATOM | 11256 | OE2 | GLU | 1671 | 40.128 | −21.804 | −0.398 | 1.00 | 27.07 |
| ATOM | 11257 | C | GLU | 1671 | 39.978 | −20.070 | 4.616 | 1.00 | 18.25 |
| ATOM | 11258 | O | GLU | 1671 | 39.164 | −20.901 | 5.016 | 1.00 | 17.58 |
| ATOM | 11259 | N | ALA | 1672 | 41.217 | −19.985 | 5.095 | 1.00 | 19.38 |
| ATOM | 11260 | CA | ALA | 1672 | 41.688 | −20.879 | 6.148 | 1.00 | 20.20 |
| ATOM | 11261 | CB | ALA | 1672 | 43.164 | −20.623 | 6.434 | 1.00 | 21.78 |
| ATOM | 11262 | C | ALA | 1672 | 40.870 | −20.710 | 7.429 | 1.00 | 21.55 |
| ATOM | 11263 | O | ALA | 1672 | 40.684 | −21.664 | 8.192 | 1.00 | 21.04 |
| ATOM | 11264 | N | ALA | 1673 | 40.382 | −19.492 | 7.654 | 1.00 | 19.63 |
| ATOM | 11265 | CA | ALA | 1673 | 39.575 | −19.175 | 8.831 | 1.00 | 19.73 |
| ATOM | 11266 | CB | ALA | 1673 | 39.398 | −17.665 | 8.952 | 1.00 | 18.51 |
| ATOM | 11267 | C | ALA | 1673 | 38.212 | −19.857 | 8.782 | 1.00 | 20.99 |
| ATOM | 11268 | O | ALA | 1673 | 37.545 | −20.010 | 9.807 | 1.00 | 19.73 |
| ATOM | 11269 | N | GLY | 1674 | 37.786 | −20.248 | 7.584 | 1.00 | 20.06 |
| ATOM | 11270 | CA | GLY | 1674 | 36.510 | −20.928 | 7.453 | 1.00 | 19.89 |
| ATOM | 11271 | C | GLY | 1674 | 35.552 | −20.324 | 6.444 | 1.00 | 18.97 |
| ATOM | 11272 | O | GLY | 1674 | 34.468 | −20.848 | 6.245 | 1.00 | 18.23 |
| ATOM | 11273 | N | ALA | 1675 | 35.936 | −19.217 | 5.818 | 1.00 | 19.69 |
| ATOM | 11274 | CA | ALA | 1675 | 35.077 | −18.589 | 4.822 | 1.00 | 20.14 |
| ATOM | 11275 | CB | ALA | 1675 | 35.736 | −17.312 | 4.292 | 1.00 | 19.92 |
| ATOM | 11276 | C | ALA | 1675 | 34.830 | −19.582 | 3.680 | 1.00 | 21.06 |
| ATOM | 11277 | O | ALA | 1675 | 35.772 | −20.194 | 3.175 | 1.00 | 22.86 |
| ATOM | 11278 | N | GLN | 1676 | 33.565 | −19.741 | 3.285 | 1.00 | 20.43 |
| ATOM | 11279 | CA | GLN | 1676 | 33.194 | −20.659 | 2.208 | 1.00 | 20.46 |
| ATOM | 11280 | CB | GLN | 1676 | 31.923 | −21.426 | 2.578 | 1.00 | 21.87 |
| ATOM | 11281 | CG | GLN | 1676 | 32.049 | −22.250 | 3.854 | 1.00 | 23.52 |
| ATOM | 11282 | CD | GLN | 1676 | 30.805 | −23.067 | 4.165 | 1.00 | 26.30 |
| ATOM | 11283 | OE1 | GLN | 1676 | 30.470 | −24.007 | 3.449 | 1.00 | 30.08 |
| ATOM | 11284 | NE2 | GLN | 1676 | 30.116 | −22.707 | 5.240 | 1.00 | 28.15 |
| ATOM | 11285 | C | GLN | 1676 | 32.982 | −19.938 | 0.879 | 1.00 | 19.59 |
| ATOM | 11286 | O | GLN | 1676 | 32.822 | −20.572 | −0.163 | 1.00 | 20.05 |
| ATOM | 11287 | N | LEU | 1677 | 32.973 | −18.612 | 0.928 | 1.00 | 20.26 |
| ATOM | 11288 | CA | LEU | 1677 | 32.797 | −17.792 | −0.257 | 1.00 | 20.79 |
| ATOM | 11289 | CB | LEU | 1677 | 31.314 | −17.476 | −0.467 | 1.00 | 22.99 |
| ATOM | 11290 | CG | LEU | 1677 | 30.499 | −18.528 | −1.225 | 1.00 | 25.39 |
| ATOM | 11291 | CD1 | LEU | 1677 | 29.007 | −18.325 | −0.979 | 1.00 | 26.62 |
| ATOM | 11292 | CD2 | LEU | 1677 | 30.821 | −18.426 | −2.711 | 1.00 | 25.79 |
| ATOM | 11293 | C | LEU | 1677 | 33.570 | −16.496 | −0.092 | 1.00 | 21.45 |
| ATOM | 11294 | O | LEU | 1677 | 33.826 | −16.058 | 1.030 | 1.00 | 20.88 |
| ATOM | 11295 | N | LEU | 1678 | 33.944 | −15.881 | −1.210 | 1.00 | 17.59 |
| ATOM | 11296 | CA | LEU | 1678 | 34.675 | −14.617 | −1.168 | 1.00 | 20.03 |
| ATOM | 11297 | CB | LEU | 1678 | 36.189 | −14.847 | −1.284 | 1.00 | 19.25 |
| ATOM | 11298 | CG | LEU | 1678 | 37.026 | −13.574 | −1.442 | 1.00 | 21.72 |
| ATOM | 11299 | CD1 | LEU | 1678 | 36.892 | −12.699 | −0.207 | 1.00 | 19.17 |
| ATOM | 11300 | CD2 | LEU | 1678 | 38.487 | −13.937 | −1.671 | 1.00 | 20.95 |
| ATOM | 11301 | C | LEU | 1678 | 34.238 | −13.695 | −2.288 | 1.00 | 19.21 |
| ATOM | 11302 | O | LEU | 1678 | 34.090 | −14.119 | −3.432 | 1.00 | 20.80 |
| ATOM | 11303 | N | VAL | 1679 | 34.026 | −12.433 | −1.947 | 1.00 | 19.50 |
| ATOM | 11304 | CA | VAL | 1679 | 33.639 | −11.435 | −2.929 | 1.00 | 20.39 |
| ATOM | 11305 | CB | VAL | 1679 | 32.395 | −10.630 | −2.476 | 1.00 | 18.97 |
| ATOM | 11306 | CG1 | VAL | 1679 | 32.202 | −9.423 | −3.389 | 1.00 | 19.05 |
| ATOM | 11307 | CG2 | VAL | 1679 | 31.145 | −11.517 | −2.518 | 1.00 | 17.14 |
| ATOM | 11308 | C | VAL | 1679 | 34.799 | −10.462 | −3.121 | 1.00 | 22.00 |
| ATOM | 11309 | O | VAL | 1679 | 35.351 | −9.935 | −2.145 | 1.00 | 20.40 |
| ATOM | 11310 | N | LEU | 1680 | 35.174 | −10.249 | −4.380 | 1.00 | 22.33 |
| ATOM | 11311 | CA | LEU | 1680 | 36.250 | −9.325 | −4.745 | 1.00 | 24.09 |
| ATOM | 11312 | CB | LEU | 1680 | 37.267 | −10.011 | −5.666 | 1.00 | 25.86 |
| ATOM | 11313 | CG | LEU | 1680 | 38.561 | −10.510 | −5.030 | 1.00 | 28.75 |
| ATOM | 11314 | CD1 | LEU | 1680 | 39.434 | −11.167 | −6.092 | 1.00 | 28.15 |
| ATOM | 11315 | CD2 | LEU | 1680 | 39.295 | −9.338 | −4.388 | 1.00 | 28.06 |
| ATOM | 11316 | C | LEU | 1680 | 35.631 | −8.147 | −5.483 | 1.00 | 23.79 |
| ATOM | 11317 | O | LEU | 1680 | 34.984 | −8.338 | −6.508 | 1.00 | 26.35 |
| ATOM | 11318 | N | GLU | 1681 | 35.829 | −6.934 | −4.976 | 1.00 | 23.88 |
| ATOM | 11319 | CA | GLU | 1681 | 35.252 | −5.753 | −5.613 | 1.00 | 26.33 |
| ATOM | 11320 | CB | GLU | 1681 | 34.293 | −5.065 | −4.636 | 1.00 | 23.82 |
| ATOM | 11321 | CG | GLU | 1681 | 33.793 | −3.695 | −5.074 | 1.00 | 25.12 |
| ATOM | 11322 | CD | GLU | 1681 | 32.590 | −3.233 | −4.264 | 1.00 | 25.80 |
| ATOM | 11323 | OE1 | GLU | 1681 | 32.420 | −3.717 | −3.125 | 1.00 | 26.14 |
| ATOM | 11324 | OE2 | GLU | 1681 | 31.820 | −2.386 | −4.760 | 1.00 | 27.76 |
| ATOM | 11325 | C | GLU | 1681 | 36.272 | −4.742 | −6.137 | 1.00 | 27.77 |
| ATOM | 11326 | O | GLU | 1681 | 37.181 | −4.327 | −5.417 | 1.00 | 28.68 |
| ATOM | 11327 | N | CYS | 1682 | 36.107 | −4.354 | −7.399 | 1.00 | 29.09 |
| ATOM | 11328 | CA | CYS | 1682 | 36.982 | −3.378 | −8.045 | 1.00 | 30.51 |
| ATOM | 11329 | CB | CYS | 1682 | 36.562 | −1.968 | −7.639 | 1.00 | 30.60 |
| ATOM | 11330 | SG | CYS | 1682 | 34.887 | −1.555 | −8.174 | 1.00 | 32.38 |
| ATOM | 11331 | C | CYS | 1682 | 38.463 | −3.582 | −7.760 | 1.00 | 31.19 |
| ATOM | 11332 | O | CYS | 1682 | 39.075 | −2.841 | −6.984 | 1.00 | 32.08 |
| ATOM | 11333 | N | VAL | 1683 | 39.029 | −4.593 | −8.405 | 1.00 | 31.35 |
| ATOM | 11334 | CA | VAL | 1683 | 40.436 | −4.922 | −8.248 | 1.00 | 32.78 |
| ATOM | 11335 | CB | VAL | 1683 | 40.600 | −6.146 | −7.312 | 1.00 | 33.52 |
| ATOM | 11336 | CG1 | VAL | 1683 | 40.443 | −7.443 | −8.094 | 1.00 | 33.50 |
| ATOM | 11337 | CG2 | VAL | 1683 | 41.927 | −6.085 | −6.596 | 1.00 | 34.74 |
| ATOM | 11338 | C | VAL | 1683 | 40.971 | −5.249 | −9.645 | 1.00 | 33.45 |
| ATOM | 11339 | O | VAL | 1683 | 40.218 | −5.684 | −10.516 | 1.00 | 33.44 |
| ATOM | 11340 | N | PRO | 1684 | 42.274 | −5.024 | −9.887 | 1.00 | 33.91 |
| ATOM | 11341 | CD | PRO | 1684 | 43.339 | −4.567 | −8.979 | 1.00 | 33.50 |
| ATOM | 11342 | CA | PRO | 1684 | 42.817 | −5.330 | −11.216 | 1.00 | 33.77 |
| ATOM | 11343 | CB | PRO | 1684 | 44.307 | −5.011 | −11.066 | 1.00 | 34.17 |
| ATOM | 11344 | CG | PRO | 1684 | 44.558 | −5.205 | −9.598 | 1.00 | 34.44 |
| ATOM | 11345 | C | PRO | 1684 | 42.557 | −6.783 | −11.595 | 1.00 | 33.67 |
| ATOM | 11346 | O | PRO | 1684 | 42.807 | −7.693 | −10.806 | 1.00 | 33.93 |
| ATOM | 11347 | N | VAL | 1685 | 42.043 | −6.987 | −12.802 | 1.00 | 33.35 |
| ATOM | 11348 | CA | VAL | 1685 | 41.731 | −8.321 | −13.301 | 1.00 | 33.55 |
| ATOM | 11349 | CB | VAL | 1685 | 41.535 | −8.305 | −14.827 | 1.00 | 33.86 |
| ATOM | 11350 | CG1 | VAL | 1685 | 40.993 | −9.650 | −15.296 | 1.00 | 33.59 |
| ATOM | 11351 | CG2 | VAL | 1685 | 40.601 | −7.175 | −15.216 | 1.00 | 32.41 |
| ATOM | 11352 | C | VAL | 1685 | 42.817 | −9.343 | −12.976 | 1.00 | 34.90 |
| ATOM | 11353 | O | VAL | 1685 | 42.529 | −10.426 | −12.463 | 1.00 | 33.21 |
| ATOM | 11354 | N | GLU | 1686 | 44.065 | −8.994 | −13.278 | 1.00 | 36.15 |
| ATOM | 11355 | CA | GLU | 1686 | 45.185 | −9.893 | −13.024 | 1.00 | 37.45 |
| ATOM | 11356 | CB | GLU | 1686 | 46.512 | −9.245 | −13.449 | 1.00 | 40.77 |
| ATOM | 11357 | CG | GLU | 1686 | 46.516 | −7.719 | −13.470 | 1.00 | 45.35 |
| ATOM | 11358 | CD | GLU | 1686 | 45.716 | −7.144 | −14.631 | 1.00 | 47.26 |
| ATOM | 11359 | OE1 | GLU | 1686 | 45.988 | −7.521 | −15.793 | 1.00 | 48.37 |
| ATOM | 11360 | OE2 | GLU | 1686 | 44.820 | −6.310 | −14.383 | 1.00 | 49.33 |
| ATOM | 11361 | C | GLU | 1686 | 45.259 | −10.326 | −11.565 | 1.00 | 35.59 |
| ATOM | 11362 | O | GLU | 1686 | 45.636 | −11.460 | −11.269 | 1.00 | 35.39 |
| ATOM | 11363 | N | LEU | 1687 | 44.896 | −9.427 | −10.656 | 1.00 | 34.93 |
| ATOM | 11364 | CA | LEU | 1687 | 44.926 | −9.743 | −9.234 | 1.00 | 34.56 |
| ATOM | 11365 | CB | LEU | 1687 | 44.773 | −8.473 | −8.400 | 1.00 | 36.48 |
| ATOM | 11366 | CG | LEU | 1687 | 45.413 | −8.477 | −7.007 | 1.00 | 38.23 |
| ATOM | 11367 | CD1 | LEU | 1687 | 45.086 | −7.168 | −6.311 | 1.00 | 39.44 |
| ATOM | 11368 | CD2 | LEU | 1687 | 44.909 | −9.646 | −6.189 | 1.00 | 40.16 |
| ATOM | 11369 | C | LEU | 1687 | 43.783 | −10.706 | −8.934 | 1.00 | 33.56 |
| ATOM | 11370 | O | LEU | 1687 | 43.940 | −11.656 | −8.166 | 1.00 | 33.33 |
| ATOM | 11371 | N | ALA | 1688 | 42.633 | −10.455 | −9.552 | 1.00 | 33.02 |
| ATOM | 11372 | CA | ALA | 1688 | 41.462 | −11.303 | −9.374 | 1.00 | 32.03 |
| ATOM | 11373 | CB | ALA | 1688 | 40.285 | −10.748 | −10.174 | 1.00 | 31.82 |
| ATOM | 11374 | C | ALA | 1688 | 41.798 | −12.713 | −9.841 | 1.00 | 31.88 |
| ATOM | 11375 | O | ALA | 1688 | 41.306 | −13.698 | −9.288 | 1.00 | 30.38 |
| ATOM | 11376 | N | LYS | 1689 | 42.642 | −12.799 | −10.865 | 1.00 | 32.10 |
| ATOM | 11377 | CA | LYS | 1689 | 43.065 | −14.080 | −11.420 | 1.00 | 32.59 |
| ATOM | 11378 | CB | LYS | 1689 | 43.921 | −13.852 | −12.667 | 1.00 | 35.33 |
| ATOM | 11379 | CG | LYS | 1689 | 43.301 | −12.915 | −13.688 | 1.00 | 39.20 |
| ATOM | 11380 | CD | LYS | 1689 | 44.230 | −12.702 | −14.875 | 1.00 | 43.71 |
| ATOM | 11381 | CE | LYS | 1689 | 43.639 | −11.725 | −15.875 | 1.00 | 44.72 |
| ATOM | 11382 | NZ | LYS | 1689 | 44.550 | −11.492 | −17.027 | 1.00 | 46.98 |
| ATOM | 11383 | C | LYS | 1689 | 43.877 | −14.854 | −10.392 | 1.00 | 30.77 |
| ATOM | 11384 | O | LYS | 1689 | 43.635 | −16.040 | −10.158 | 1.00 | 30.60 |
| ATOM | 11385 | N | ARG | 1690 | 44.850 | −14.172 | −9.793 | 1.00 | 30.36 |
| ATOM | 11386 | CA | ARG | 1690 | 45.715 | −14.773 | −8.782 | 1.00 | 30.78 |
| ATOM | 11387 | CB | ARG | 1690 | 46.706 | −13.738 | −8.236 | 1.00 | 32.24 |
| ATOM | 11388 | CG | ARG | 1690 | 48.153 | −13.968 | −8.653 | 1.00 | 35.21 |
| ATOM | 11389 | CD | ARG | 1690 | 49.122 | −13.194 | −7.766 | 1.00 | 35.72 |
| ATOM | 11390 | NE | ARG | 1690 | 48.987 | −11.749 | −7.919 | 1.00 | 38.51 |
| ATOM | 11391 | CZ | ARG | 1690 | 49.548 | −10.854 | −7.110 | 1.00 | 38.81 |
| ATOM | 11392 | NH1 | ARG | 1690 | 50.285 | −11.253 | −6.082 | 1.00 | 38.33 |
| ATOM | 11393 | NH2 | ARG | 1690 | 49.374 | −9.557 | −7.331 | 1.00 | 38.70 |
| ATOM | 11394 | C | ARG | 1690 | 44.895 | −15.329 | −7.627 | 1.00 | 29.79 |
| ATOM | 11395 | O | ARG | 1690 | 45.018 | −16.503 | −7.271 | 1.00 | 29.49 |
| ATOM | 11396 | N | ILE | 1691 | 44.059 | −14.475 | −7.044 | 1.00 | 29.53 |
| ATOM | 11397 | CA | ILE | 1691 | 43.223 | −14.874 | −5.920 | 1.00 | 27.99 |
| ATOM | 11398 | CB | ILE | 1691 | 42.368 | −13.687 | −5.428 | 1.00 | 28.66 |
| ATOM | 11399 | CG2 | ILE | 1691 | 41.431 | −14.141 | −4.310 | 1.00 | 29.07 |
| ATOM | 11400 | CG1 | ILE | 1691 | 43.290 | −12.569 | −4.939 | 1.00 | 27.59 |
| ATOM | 11401 | CD1 | ILE | 1691 | 42.571 | −11.367 | −4.367 | 1.00 | 30.13 |
| ATOM | 11402 | C | ILE | 1691 | 42.322 | −16.053 | −6.278 | 1.00 | 28.14 |
| ATOM | 11403 | O | ILE | 1691 | 42.267 | −17.044 | −5.551 | 1.00 | 28.03 |
| ATOM | 11404 | N | THR | 1692 | 41.635 | −15.952 | −7.412 | 1.00 | 27.85 |
| ATOM | 11405 | CA | THR | 1692 | 40.743 | −17.016 | −7.852 | 1.00 | 28.15 |
| ATOM | 11406 | CB | THR | 1692 | 40.089 | −16.670 | −9.207 | 1.00 | 27.92 |
| ATOM | 11407 | OG1 | THR | 1692 | 39.305 | −15.477 | −9.070 | 1.00 | 30.45 |
| ATOM | 11408 | CG2 | THR | 1692 | 39.192 | −17.799 | −9.672 | 1.00 | 24.79 |
| ATOM | 11409 | C | THR | 1692 | 41.468 | −18.352 | −7.975 | 1.00 | 28.88 |
| ATOM | 11410 | O | THR | 1692 | 40.967 | −19.378 | −7.523 | 1.00 | 27.96 |
| ATOM | 11411 | N | GLU | 1693 | 42.649 | −18.346 | −8.585 | 1.00 | 30.86 |
| ATOM | 11412 | CA | GLU | 1693 | 43.410 | −19.582 | −8.739 | 1.00 | 31.66 |
| ATOM | 11413 | CB | GLU | 1693 | 44.509 | −19.403 | −9.788 | 1.00 | 34.46 |
| ATOM | 11414 | CG | GLU | 1693 | 43.985 | −19.142 | −11.188 | 1.00 | 39.25 |
| ATOM | 11415 | CD | GLU | 1693 | 45.098 | −18.913 | −12.193 | 1.00 | 41.31 |
| ATOM | 11416 | OE1 | GLU | 1693 | 45.841 | −17.920 | −12.046 | 1.00 | 42.03 |
| ATOM | 11417 | OE2 | GLU | 1693 | 45.227 | −19.728 | −13.133 | 1.00 | 44.22 |
| ATOM | 11418 | C | GLU | 1693 | 44.031 | −20.022 | −7.417 | 1.00 | 31.18 |
| ATOM | 11419 | O | GLU | 1693 | 44.220 | −21.219 | −7.180 | 1.00 | 32.59 |
| ATOM | 11420 | N | ALA | 1694 | 44.339 | −19.055 | −6.556 | 1.00 | 29.06 |
| ATOM | 11421 | CA | ALA | 1694 | 44.954 | −19.349 | −5.263 | 1.00 | 28.83 |
| ATOM | 11422 | CB | ALA | 1694 | 45.568 | −18.077 | −4.681 | 1.00 | 28.10 |
| ATOM | 11423 | C | ALA | 1694 | 43.997 | −19.975 | −4.252 | 1.00 | 28.71 |
| ATOM | 11424 | O | ALA | 1694 | 44.398 | −20.833 | −3.463 | 1.00 | 27.81 |
| ATOM | 11425 | N | LEU | 1695 | 42.734 | −19.556 | −4.273 | 1.00 | 28.34 |
| ATOM | 11426 | CA | LEU | 1695 | 41.750 | −20.103 | −3.336 | 1.00 | 28.55 |
| ATOM | 11427 | CB | LEU | 1695 | 40.729 | −19.035 | −2.936 | 1.00 | 28.01 |
| ATOM | 11428 | CG | LEU | 1695 | 41.242 | −17.849 | −2.121 | 1.00 | 30.89 |
| ATOM | 11429 | CD1 | LEU | 1695 | 40.062 | −16.980 | −1.700 | 1.00 | 27.57 |
| ATOM | 11430 | CD2 | LEU | 1695 | 41.989 | −18.348 | −0.898 | 1.00 | 32.34 |
| ATOM | 11431 | C | LEU | 1695 | 41.002 | −21.306 | −3.889 | 1.00 | 26.44 |
| ATOM | 11432 | O | LEU | 1695 | 40.848 | −21.452 | −5.099 | 1.00 | 27.20 |
| ATOM | 11433 | N | ALA | 1696 | 40.541 | −22.168 | −2.991 | 1.00 | 24.30 |
| ATOM | 11434 | CA | ALA | 1696 | 39.790 | −23.346 | −3.391 | 1.00 | 23.55 |
| ATOM | 11435 | CB | ALA | 1696 | 40.026 | −24.479 | −2.413 | 1.00 | 21.99 |
| ATOM | 11436 | C | ALA | 1696 | 38.313 | −22.983 | −3.416 | 1.00 | 23.10 |
| ATOM | 11437 | O | ALA | 1696 | 37.562 | −23.440 | −4.279 | 1.00 | 24.16 |
| ATOM | 11438 | N | ILE | 1697 | 37.898 | −22.155 | −2.464 | 1.00 | 21.53 |
| ATOM | 11439 | CA | ILE | 1697 | 36.506 | −21.734 | −2.391 | 1.00 | 20.71 |
| ATOM | 11440 | CB | ILE | 1697 | 36.211 | −20.965 | −1.082 | 1.00 | 18.95 |
| ATOM | 11441 | CG2 | ILE | 1697 | 36.340 | −21.897 | 0.113 | 1.00 | 17.11 |
| ATOM | 11442 | CG1 | ILE | 1697 | 37.158 | −19.776 | −0.955 | 1.00 | 18.41 |
| ATOM | 11443 | CD1 | ILE | 1697 | 36.776 | −18.806 | 0.147 | 1.00 | 20.97 |
| ATOM | 11444 | C | ILE | 1697 | 36.185 | −20.823 | −3.571 | 1.00 | 20.34 |
| ATOM | 11445 | O | ILE | 1697 | 37.073 | −20.206 | −4.155 | 1.00 | 20.05 |
| ATOM | 11446 | N | PRO | 1698 | 34.903 | −20.739 | −3.948 | 1.00 | 21.40 |
| ATOM | 11447 | CD | PRO | 1698 | 33.767 | −21.568 | −3.513 | 1.00 | 20.73 |
| ATOM | 11448 | CA | PRO | 1698 | 34.541 | −19.878 | −5.074 | 1.00 | 20.26 |
| ATOM | 11449 | CB | PRO | 1698 | 33.102 | −20.302 | −5.393 | 1.00 | 21.48 |
| ATOM | 11450 | CG | PRO | 1698 | 32.594 | −20.847 | −4.113 | 1.00 | 22.95 |
| ATOM | 11451 | C | PRO | 1698 | 34.689 | −18.390 | −4.769 | 1.00 | 20.91 |
| ATOM | 11452 | O | PRO | 1698 | 34.431 | −17.932 | −3.650 | 1.00 | 19.65 |
| ATOM | 11453 | N | VAL | 1699 | 35.138 | −17.648 | −5.770 | 1.00 | 18.53 |
| ATOM | 11454 | CA | VAL | 1699 | 35.337 | −16.216 | −5.637 | 1.00 | 19.92 |
| ATOM | 11455 | CB | VAL | 1699 | 36.802 | −15.840 | −5.991 | 1.00 | 20.40 |
| ATOM | 11456 | CG1 | VAL | 1699 | 37.002 | −14.335 | −5.923 | 1.00 | 18.70 |
| ATOM | 11457 | CG2 | VAL | 1699 | 37.751 | −16.534 | −5.028 | 1.00 | 18.50 |
| ATOM | 11458 | C | VAL | 1699 | 34.366 | −15.480 | −6.560 | 1.00 | 21.83 |
| ATOM | 11459 | O | VAL | 1699 | 34.374 | −15.672 | −7.779 | 1.00 | 22.52 |
| ATOM | 11460 | N | ILE | 1700 | 33.517 | −14.648 | −5.967 | 1.00 | 22.27 |
| ATOM | 11461 | CA | ILE | 1700 | 32.534 | −13.886 | −6.722 | 1.00 | 22.16 |
| ATOM | 11462 | CB | ILE | 1700 | 31.211 | −13.759 | −5.940 | 1.00 | 21.79 |
| ATOM | 11463 | CG2 | ILE | 1700 | 30.243 | −12.873 | −6.700 | 1.00 | 22.27 |
| ATOM | 11464 | CG1 | ILE | 1700 | 30.617 | −15.150 | −5.709 | 1.00 | 22.01 |
| ATOM | 11465 | CD1 | ILE | 1700 | 29.416 | −15.157 | −4.789 | 1.00 | 24.61 |
| ATOM | 11466 | C | ILE | 1700 | 33.100 | −12.504 | −6.963 | 1.00 | 21.79 |
| ATOM | 11467 | O | ILE | 1700 | 33.537 | −11.828 | −6.030 | 1.00 | 21.47 |
| ATOM | 11468 | N | GLY | 1701 | 33.096 | −12.079 | −8.218 | 1.00 | 21.16 |
| ATOM | 11469 | CA | GLY | 1701 | 33.635 | −10.773 | −8.526 | 1.00 | 20.63 |
| ATOM | 11470 | C | GLY | 1701 | 32.633 | −9.714 | −8.929 | 1.00 | 21.51 |
| ATOM | 11471 | O | GLY | 1701 | 31.571 | −10.003 | −9.467 | 1.00 | 22.89 |
| ATOM | 11472 | N | ILE | 1702 | 32.990 | −8.470 | −8.642 | 1.00 | 24.15 |
| ATOM | 11473 | CA | ILE | 1702 | 32.184 | −7.309 | −8.988 | 1.00 | 25.08 |
| ATOM | 11474 | CB | ILE | 1702 | 31.275 | −6.866 | −7.804 | 1.00 | 25.11 |
| ATOM | 11475 | CG2 | ILE | 1702 | 32.059 | −6.838 | −6.504 | 1.00 | 24.79 |
| ATOM | 11476 | CG1 | ILE | 1702 | 30.679 | −5.490 | −8.083 | 1.00 | 28.41 |
| ATOM | 11477 | CD1 | ILE | 1702 | 29.632 | −5.485 | −9.157 | 1.00 | 30.16 |
| ATOM | 11478 | C | ILE | 1702 | 33.209 | −6.228 | −9.321 | 1.00 | 25.61 |
| ATOM | 11479 | O | ILE | 1702 | 33.728 | −5.553 | −8.437 | 1.00 | 25.75 |
| ATOM | 11480 | N | GLY | 1703 | 33.515 | −6.084 | −10.608 | 1.00 | 27.06 |
| ATOM | 11481 | CA | GLY | 1703 | 34.501 | −5.105 | −11.017 | 1.00 | 24.09 |
| ATOM | 11482 | C | GLY | 1703 | 35.884 | −5.717 | −10.904 | 1.00 | 25.54 |
| ATOM | 11483 | O | GLY | 1703 | 36.873 | −5.017 | −10.680 | 1.00 | 27.11 |
| ATOM | 11484 | N | ALA | 1704 | 35.945 | −7.037 | −11.054 | 1.00 | 24.77 |
| ATOM | 11485 | CA | ALA | 1704 | 37.204 | −7.767 | −10.970 | 1.00 | 26.19 |
| ATOM | 11486 | CB | ALA | 1704 | 37.187 | −8.691 | −9.757 | 1.00 | 24.95 |
| ATOM | 11487 | C | ALA | 1704 | 37.454 | −8.582 | −12.238 | 1.00 | 26.93 |
| ATOM | 11488 | O | ALA | 1704 | 38.294 | −9.482 | −12.250 | 1.00 | 27.02 |
| ATOM | 11489 | N | GLY | 1705 | 36.725 | −8.266 | −13.303 | 1.00 | 27.42 |
| ATOM | 11490 | CA | GLY | 1705 | 36.895 | −8.993 | −14.545 | 1.00 | 27.32 |
| ATOM | 11491 | C | GLY | 1705 | 36.120 | −10.297 | −14.548 | 1.00 | 27.69 |
| ATOM | 11492 | O | GLY | 1705 | 35.490 | −10.653 | −13.551 | 1.00 | 28.16 |
| ATOM | 11493 | N | ASN | 1706 | 36.170 | −11.017 | −15.666 | 1.00 | 26.41 |
| ATOM | 11494 | CA | ASN | 1706 | 35.452 | −12.279 | −15.801 | 1.00 | 26.15 |
| ATOM | 11495 | CB | ASN | 1706 | 34.909 | −12.423 | −17.230 | 1.00 | 28.81 |
| ATOM | 11496 | CG | ASN | 1706 | 36.007 | −12.410 | −18.283 | 1.00 | 30.05 |
| ATOM | 11497 | OD1 | ASN | 1706 | 35.750 | −12.663 | −19.462 | 1.00 | 32.36 |
| ATOM | 11498 | ND2 | ASN | 1706 | 37.231 | −12.111 | −17.866 | 1.00 | 27.42 |
| ATOM | 11499 | C | ASN | 1706 | 36.306 | −13.492 | −15.452 | 1.00 | 27.06 |
| ATOM | 11500 | O | ASN | 1706 | 36.005 | −14.609 | −15.874 | 1.00 | 28.09 |
| ATOM | 11501 | N | VAL | 1707 | 37.358 | −13.268 | −14.669 | 1.00 | 25.49 |
| ATOM | 11502 | CA | VAL | 1707 | 38.270 | −14.335 | −14.258 | 1.00 | 27.59 |
| ATOM | 11503 | CB | VAL | 1707 | 39.700 | −13.780 | −14.047 | 1.00 | 29.11 |
| ATOM | 11504 | CG1 | VAL | 1707 | 40.656 | −14.914 | −13.711 | 1.00 | 33.83 |
| ATOM | 11505 | CG2 | VAL | 1707 | 40.169 | −13.053 | −15.299 | 1.00 | 30.80 |
| ATOM | 11506 | C | VAL | 1707 | 37.815 | −15.016 | −12.967 | 1.00 | 26.92 |
| ATOM | 11507 | O | VAL | 1707 | 38.311 | −16.088 | −12.612 | 1.00 | 26.60 |
| ATOM | 11508 | N | THR | 1708 | 36.878 | −14.389 | −12.263 | 1.00 | 24.26 |
| ATOM | 11509 | CA | THR | 1708 | 36.364 | −14.948 | −11.019 | 1.00 | 23.16 |
| ATOM | 11510 | CB | THR | 1708 | 35.625 | −13.867 | −10.202 | 1.00 | 22.08 |
| ATOM | 11511 | OG1 | THR | 1708 | 34.731 | −13.145 | −11.059 | 1.00 | 20.76 |
| ATOM | 11512 | CG2 | THR | 1708 | 36.620 | −12.894 | −9.592 | 1.00 | 22.51 |
| ATOM | 11513 | C | THR | 1708 | 35.427 | −16.122 | −11.300 | 1.00 | 23.44 |
| ATOM | 11514 | O | THR | 1708 | 34.965 | −16.304 | −12.427 | 1.00 | 24.03 |
| ATOM | 11515 | N | ASP | 1709 | 35.157 | −16.921 | −10.271 | 1.00 | 23.18 |
| ATOM | 11516 | CA | ASP | 1709 | 34.299 | −18.098 | −10.398 | 1.00 | 24.19 |
| ATOM | 11517 | CB | ASP | 1709 | 34.425 | −18.954 | −9.139 | 1.00 | 26.39 |
| ATOM | 11518 | CG | ASP | 1709 | 35.857 | −19.362 | −8.861 | 1.00 | 28.72 |
| ATOM | 11519 | OD1 | ASP | 1709 | 36.395 | −20.179 | −9.636 | 1.00 | 28.85 |
| ATOM | 11520 | OD2 | ASP | 1709 | 36.440 | −18.858 | −7.876 | 1.00 | 27.39 |
| ATOM | 11521 | C | ASP | 1709 | 32.843 | −17.708 | −10.608 | 1.00 | 24.34 |
| ATOM | 11522 | O | ASP | 1709 | 32.054 | −18.461 | −11.182 | 1.00 | 23.59 |
| ATOM | 11523 | N | GLY | 1710 | 32.499 | −16.521 | −10.127 | 1.00 | 22.85 |
| ATOM | 11524 | CA | GLY | 1710 | 31.143 | −16.032 | −10.261 | 1.00 | 21.26 |
| ATOM | 11525 | C | GLY | 1710 | 31.131 | −14.522 | −10.289 | 1.00 | 19.07 |
| ATOM | 11526 | O | GLY | 1710 | 32.168 | −13.878 | −10.113 | 1.00 | 18.22 |
| ATOM | 11527 | N | GLN | 1711 | 29.949 | −13.956 | −10.505 | 1.00 | 18.74 |
| ATOM | 11528 | CA | GLN | 1711 | 29.790 | −12.510 | −10.570 | 1.00 | 18.89 |
| ATOM | 11529 | CB | GLN | 1711 | 29.502 | −12.074 | −12.007 | 1.00 | 18.46 |
| ATOM | 11530 | CG | GLN | 1711 | 30.592 | −12.373 | −13.018 | 1.00 | 15.95 |
| ATOM | 11531 | CD | GLN | 1711 | 31.848 | −11.589 | −12.758 | 1.00 | 19.23 |
| ATOM | 11532 | OE1 | GLN | 1711 | 31.796 | −10.409 | −12.418 | 1.00 | 20.10 |
| ATOM | 11533 | NE2 | GLN | 1711 | 32.995 | −12.237 | −12.930 | 1.00 | 21.35 |
| ATOM | 11534 | C | GLN | 1711 | 28.623 | −12.056 | −9.712 | 1.00 | 19.28 |
| ATOM | 11535 | O | GLN | 1711 | 27.756 | −12.852 | −9.359 | 1.00 | 18.49 |
| ATOM | 11536 | N | ILE | 1712 | 28.605 | −10.767 | −9.401 | 1.00 | 21.16 |
| ATOM | 11537 | CA | ILE | 1712 | 27.522 | −10.172 | −8.634 | 1.00 | 23.23 |
| ATOM | 11538 | CB | ILE | 1712 | 27.772 | −10.262 | −7.109 | 1.00 | 24.48 |
| ATOM | 11539 | CG2 | ILE | 1712 | 28.930 | −9.342 | −6.706 | 1.00 | 25.08 |
| ATOM | 11540 | CG1 | ILE | 1712 | 26.492 | −9.870 | −6.365 | 1.00 | 25.35 |
| ATOM | 11541 | CD1 | ILE | 1712 | 26.463 | −10.280 | −4.898 | 1.00 | 29.02 |
| ATOM | 11542 | C | ILE | 1712 | 27.374 | −8.710 | −9.055 | 1.00 | 24.32 |
| ATOM | 11543 | O | ILE | 1712 | 28.328 | −8.085 | −9.539 | 1.00 | 23.00 |
| ATOM | 11544 | N | LEU | 1713 | 26.172 | −8.169 | −8.888 | 1.00 | 24.37 |
| ATOM | 11545 | CA | LEU | 1713 | 25.914 | −6.778 | −9.249 | 1.00 | 27.03 |
| ATOM | 11546 | CB | LEU | 1713 | 25.837 | −6.650 | −10.772 | 1.00 | 30.07 |
| ATOM | 11547 | CG | LEU | 1713 | 26.489 | −5.428 | −11.427 | 1.00 | 33.56 |
| ATOM | 11548 | CD1 | LEU | 1713 | 26.318 | −5.532 | −12.934 | 1.00 | 36.19 |
| ATOM | 11549 | CD2 | LEU | 1713 | 25.851 | −4.137 | −10.908 | 1.00 | 36.32 |
| ATOM | 11550 | C | LEU | 1713 | 24.609 | −6.296 | −8.620 | 1.00 | 26.45 |
| ATOM | 11551 | O | LEU | 1713 | 23.723 | −7.099 | −8.338 | 1.00 | 23.98 |
| ATOM | 11552 | N | VAL | 1714 | 24.509 | −4.989 | −8.386 | 1.00 | 25.79 |
| ATOM | 11553 | CA | VAL | 1714 | 23.299 | −4.410 | −7.808 | 1.00 | 25.33 |
| ATOM | 11554 | CB | VAL | 1714 | 23.522 | −2.943 | −7.363 | 1.00 | 26.34 |
| ATOM | 11555 | CG1 | VAL | 1714 | 22.245 | −2.379 | −6.750 | 1.00 | 25.51 |
| ATOM | 11556 | CG2 | VAL | 1714 | 24.660 | −2.878 | −6.351 | 1.00 | 27.39 |
| ATOM | 11557 | C | VAL | 1714 | 22.215 | −4.466 | −8.886 | 1.00 | 24.33 |
| ATOM | 11558 | O | VAL | 1714 | 22.379 | −3.913 | −9.978 | 1.00 | 21.59 |
| ATOM | 11559 | N | MET | 1715 | 21.115 | −5.146 | −8.573 | 1.00 | 22.29 |
| ATOM | 11560 | CA | MET | 1715 | 20.026 | −5.302 | −9.521 | 1.00 | 20.64 |
| ATOM | 11561 | CB | MET | 1715 | 18.855 | −6.059 | −8.881 | 1.00 | 18.66 |
| ATOM | 11562 | CG | MET | 1715 | 18.253 | −5.362 | −7.667 | 1.00 | 16.03 |
| ATOM | 11563 | SD | MET | 1715 | 16.444 | −5.523 | −7.565 | 1.00 | 14.77 |
| ATOM | 11564 | CE | MET | 1715 | 15.937 | −4.242 | −8.684 | 1.00 | 11.94 |
| ATOM | 11565 | C | MET | 1715 | 19.519 | −3.983 | −10.101 | 1.00 | 19.82 |
| ATOM | 11566 | O | MET | 1715 | 19.002 | −3.953 | −11.213 | 1.00 | 21.61 |
| ATOM | 11567 | N | HIS | 1716 | 19.668 | −2.890 | −9.364 | 1.00 | 19.03 |
| ATOM | 11568 | CA | HIS | 1716 | 19.192 | −1.600 | −9.855 | 1.00 | 22.19 |
| ATOM | 11569 | CB | HIS | 1716 | 19.137 | −0.596 | −8.703 | 1.00 | 20.78 |
| ATOM | 11570 | CG | HIS | 1716 | 18.098 | −0.932 | −7.675 | 1.00 | 21.06 |
| ATOM | 11571 | CD2 | HIS | 1716 | 18.126 | −1.784 | −6.623 | 1.00 | 18.32 |
| ATOM | 11572 | ND1 | HIS | 1716 | 16.826 | −0.399 | −7.699 | 1.00 | 20.68 |
| ATOM | 11573 | CE1 | HIS | 1716 | 16.116 | −0.907 | −6.707 | 1.00 | 18.23 |
| ATOM | 11574 | NE2 | HIS | 1716 | 16.882 | −1.750 | −6.039 | 1.00 | 21.81 |
| ATOM | 11575 | C | HIS | 1716 | 20.035 | −1.067 | −11.018 | 1.00 | 22.57 |
| ATOM | 11576 | O | HIS | 1716 | 19.558 | −0.273 | −11.835 | 1.00 | 22.73 |
| ATOM | 11577 | N | ASP | 1717 | 21.287 | −1.504 | −11.097 | 1.00 | 23.70 |
| ATOM | 11578 | CA | ASP | 1717 | 22.145 | −1.089 | −12.199 | 1.00 | 26.09 |
| ATOM | 11579 | CB | ASP | 1717 | 23.615 | −1.081 | −11.769 | 1.00 | 27.37 |
| ATOM | 11580 | CG | ASP | 1717 | 23.891 | −0.070 | −10.672 | 1.00 | 27.23 |
| ATOM | 11581 | OD1 | ASP | 1717 | 23.334 | 1.040 | −10.740 | 1.00 | 28.27 |
| ATOM | 11582 | OD2 | ASP | 1717 | 24.663 | −0.386 | −9.748 | 1.00 | 27.57 |
| ATOM | 11583 | C | ASP | 1717 | 21.939 | −2.064 | −13.353 | 1.00 | 27.25 |
| ATOM | 11584 | O | ASP | 1717 | 22.022 | −1.687 | −14.522 | 1.00 | 26.75 |
| ATOM | 11585 | N | ALA | 1718 | 21.641 | −3.315 | −13.013 | 1.00 | 26.99 |
| ATOM | 11586 | CA | ALA | 1718 | 21.424 | −4.349 | −14.019 | 1.00 | 28.53 |
| ATOM | 11587 | CB | ALA | 1718 | 21.320 | −5.716 | −13.344 | 1.00 | 28.27 |
| ATOM | 11588 | C | ALA | 1718 | 20.196 | −4.099 | −14.896 | 1.00 | 28.41 |
| ATOM | 11589 | O | ALA | 1718 | 20.106 | −4.644 | −15.995 | 1.00 | 29.83 |
| ATOM | 11590 | N | PHE | 1719 | 19.258 | −3.278 | −14.422 | 1.00 | 30.20 |
| ATOM | 11591 | CA | PHE | 1719 | 18.053 | −2.968 | −15.197 | 1.00 | 29.35 |
| ATOM | 11592 | CB | PHE | 1719 | 16.797 | −3.436 | −14.458 | 1.00 | 30.92 |
| ATOM | 11593 | CG | PHE | 1719 | 16.863 | −4.866 | −14.004 | 1.00 | 31.07 |
| ATOM | 11594 | CD1 | PHE | 1719 | 17.299 | −5.863 | −14.870 | 1.00 | 34.00 |
| ATOM | 11595 | CD2 | PHE | 1719 | 16.500 | −5.215 | −12.708 | 1.00 | 33.37 |
| ATOM | 11596 | CE1 | PHE | 1719 | 17.379 | −7.187 | −14.453 | 1.00 | 33.82 |
| ATOM | 11597 | CE2 | PHE | 1719 | 16.574 | −6.537 | −12.278 | 1.00 | 34.22 |
| ATOM | 11598 | CZ | PHE | 1719 | 17.016 | −7.524 | −13.155 | 1.00 | 35.15 |
| ATOM | 11599 | C | PHE | 1719 | 17.912 | −1.484 | −15.533 | 1.00 | 30.03 |
| ATOM | 11600 | O | PHE | 1719 | 16.837 | −1.021 | −15.919 | 1.00 | 29.75 |
| ATOM | 11601 | N | GLY | 1720 | 18.999 | −0.738 | −15.391 | 1.00 | 30.77 |
| ATOM | 11602 | CA | GLY | 1720 | 18.955 | 0.680 | −15.699 | 1.00 | 29.22 |
| ATOM | 11603 | C | GLY | 1720 | 17.997 | 1.486 | −14.839 | 1.00 | 29.76 |
| ATOM | 11604 | O | GLY | 1720 | 17.524 | 2.546 | −15.258 | 1.00 | 30.07 |
| ATOM | 11605 | N | ILE | 1721 | 17.701 | 0.998 | −13.638 | 1.00 | 25.96 |
| ATOM | 11606 | CA | ILE | 1721 | 16.806 | 1.721 | −12.743 | 1.00 | 25.33 |
| ATOM | 11607 | CB | ILE | 1721 | 16.312 | 0.817 | −11.572 | 1.00 | 21.96 |
| ATOM | 11608 | CG2 | ILE | 1721 | 15.463 | 1.629 | −10.606 | 1.00 | 22.06 |
| ATOM | 11609 | CG1 | ILE | 1721 | 15.492 | −0.352 | −12.118 | 1.00 | 23.00 |
| ATOM | 11610 | CD1 | ILE | 1721 | 15.126 | −1.395 | −11.081 | 1.00 | 22.54 |
| ATOM | 11611 | C | ILE | 1721 | 17.530 | 2.940 | −12.168 | 1.00 | 25.41 |
| ATOM | 11612 | O | ILE | 1721 | 16.960 | 4.020 | −12.089 | 1.00 | 25.23 |
| ATOM | 11613 | N | THR | 1722 | 18.792 | 2.766 | −11.778 | 1.00 | 28.45 |
| ATOM | 11614 | CA | THR | 1722 | 19.563 | 3.871 | −11.206 | 1.00 | 31.68 |
| ATOM | 11615 | CB | THR | 1722 | 20.885 | 3.387 | −10.588 | 1.00 | 31.93 |
| ATOM | 11616 | OG1 | THR | 1722 | 21.778 | 2.969 | −11.626 | 1.00 | 35.74 |
| ATOM | 11617 | CG2 | THR | 1722 | 20.634 | 2.227 | −9.643 | 1.00 | 28.90 |
| ATOM | 11618 | C | THR | 1722 | 19.895 | 4.940 | −12.243 | 1.00 | 34.83 |
| ATOM | 11619 | O | THR | 1722 | 20.220 | 4.621 | −13.386 | 1.00 | 34.13 |
| ATOM | 11620 | N | GLY | 1723 | 19.811 | 6.200 | −11.816 | 1.00 | 38.15 |
| ATOM | 11621 | CA | GLY | 1723 | 20.100 | 7.342 | −12.667 | 1.00 | 44.26 |
| ATOM | 11622 | C | GLY | 1723 | 20.393 | 7.053 | −14.126 | 1.00 | 48.27 |
| ATOM | 11623 | O | GLY | 1723 | 19.534 | 6.554 | −14.857 | 1.00 | 50.07 |
| ATOM | 11624 | N | GLY | 1724 | 21.612 | 7.368 | −14.554 | 1.00 | 50.26 |
| ATOM | 11625 | CA | GLY | 1724 | 21.990 | 7.136 | −15.936 | 1.00 | 52.51 |
| ATOM | 11626 | C | GLY | 1724 | 23.414 | 6.641 | −16.078 | 1.00 | 54.09 |
| ATOM | 11627 | O | GLY | 1724 | 23.677 | 5.698 | −16.825 | 1.00 | 55.33 |
| ATOM | 11628 | N | HIS | 1725 | 24.338 | 7.275 | −15.363 | 1.00 | 55.09 |
| ATOM | 11629 | CA | HIS | 1725 | 25.742 | 6.881 | −15.429 | 1.00 | 56.44 |
| ATOM | 11630 | CB | HIS | 1725 | 26.648 | 8.096 | −15.214 | 1.00 | 58.71 |
| ATOM | 11631 | CG | HIS | 1725 | 26.616 | 9.079 | −16.342 | 1.00 | 61.40 |
| ATOM | 11632 | CD2 | HIS | 1725 | 26.415 | 10.418 | −16.354 | 1.00 | 62.22 |
| ATOM | 11633 | ND1 | HIS | 1725 | 26.821 | 8.711 | −17.654 | 1.00 | 62.27 |
| ATOM | 11634 | CE1 | HIS | 1725 | 26.748 | 9.780 | −18.426 | 1.00 | 63.11 |
| ATOM | 11635 | NE2 | HIS | 1725 | 26.503 | 10.829 | −17.662 | 1.00 | 63.16 |
| ATOM | 11636 | C | HIS | 1725 | 26.097 | 5.797 | −14.419 | 1.00 | 55.56 |
| ATOM | 11637 | O | HIS | 1725 | 26.673 | 6.078 | −13.369 | 1.00 | 55.59 |
| ATOM | 11638 | N | ILE | 1726 | 25.754 | 4.555 | −14.748 | 1.00 | 54.42 |
| ATOM | 11639 | CA | ILE | 1726 | 26.044 | 3.425 | −13.875 | 1.00 | 53.21 |
| ATOM | 11640 | CB | ILE | 1726 | 25.356 | 2.134 | −14.382 | 1.00 | 53.76 |
| ATOM | 11641 | CG2 | ILE | 1726 | 23.843 | 2.277 | −14.290 | 1.00 | 53.32 |
| ATOM | 11642 | CG1 | ILE | 1726 | 25.788 | 1.845 | −15.822 | 1.00 | 54.09 |
| ATOM | 11643 | CD1 | ILE | 1726 | 25.277 | 0.524 | −16.365 | 1.00 | 53.89 |
| ATOM | 11644 | C | ILE | 1726 | 27.554 | 3.195 | −13.821 | 1.00 | 51.83 |
| ATOM | 11645 | O | ILE | 1726 | 28.297 | 3.700 | −14.665 | 1.00 | 51.84 |
| ATOM | 11646 | N | PRO | 1727 | 28.029 | 2.431 | −12.825 | 1.00 | 49.69 |
| ATOM | 11647 | CD | PRO | 1727 | 27.283 | 1.702 | −11.785 | 1.00 | 49.25 |
| ATOM | 11648 | CA | PRO | 1727 | 29.468 | 2.172 | −12.715 | 1.00 | 48.30 |
| ATOM | 11649 | CB | PRO | 1727 | 29.566 | 1.318 | −11.453 | 1.00 | 48.75 |
| ATOM | 11650 | CG | PRO | 1727 | 28.250 | 0.611 | −11.416 | 1.00 | 49.22 |
| ATOM | 11651 | C | PRO | 1727 | 30.040 | 1.480 | −13.953 | 1.00 | 46.66 |
| ATOM | 11652 | O | PRO | 1727 | 29.368 | 0.669 | −14.589 | 1.00 | 46.75 |
| ATOM | 11653 | N | LYS | 1728 | 31.283 | 1.808 | −14.290 | 1.00 | 44.70 |
| ATOM | 11654 | CA | LYS | 1728 | 31.940 | 1.230 | −15.457 | 1.00 | 42.31 |
| ATOM | 11655 | CB | LYS | 1728 | 33.379 | 1.747 | −15.571 | 1.00 | 45.34 |
| ATOM | 11656 | CG | LYS | 1728 | 33.500 | 3.186 | −16.065 | 1.00 | 49.29 |
| ATOM | 11657 | CD | LYS | 1728 | 32.922 | 4.188 | −15.074 | 1.00 | 51.69 |
| ATOM | 11658 | CE | LYS | 1728 | 33.050 | 5.614 | −15.598 | 1.00 | 52.92 |
| ATOM | 11659 | NZ | LYS | 1728 | 32.499 | 6.622 | −14.646 | 1.00 | 54.39 |
| ATOM | 11660 | C | LYS | 1728 | 31.954 | −0.293 | −15.470 | 1.00 | 38.59 |
| ATOM | 11661 | O | LYS | 1728 | 31.994 | −0.906 | −16.536 | 1.00 | 37.57 |
| ATOM | 11662 | N | PHE | 1729 | 31.920 | −0.904 | −14.292 | 1.00 | 34.69 |
| ATOM | 11663 | CA | PHE | 1729 | 31.947 | −2.358 | −14.201 | 1.00 | 30.76 |
| ATOM | 11664 | CB | PHE | 1729 | 32.582 | −2.782 | −12.870 | 1.00 | 31.47 |
| ATOM | 11665 | CG | PHE | 1729 | 31.882 | −2.234 | −11.657 | 1.00 | 31.03 |
| ATOM | 11666 | CD1 | PHE | 1729 | 30.666 | −2.765 | −11.232 | 1.00 | 31.72 |
| ATOM | 11667 | CD2 | PHE | 1729 | 32.445 | −1.190 | −10.932 | 1.00 | 31.83 |
| ATOM | 11668 | CE1 | PHE | 1729 | 30.027 | −2.265 | −10.099 | 1.00 | 30.43 |
| ATOM | 11669 | CE2 | PHE | 1729 | 31.816 | −0.684 | −9.800 | 1.00 | 32.00 |
| ATOM | 11670 | CZ | PHE | 1729 | 30.604 | −1.222 | −9.381 | 1.00 | 32.08 |
| ATOM | 11671 | C | PHE | 1729 | 30.571 | −2.999 | −14.346 | 1.00 | 27.80 |
| ATOM | 11672 | O | PHE | 1729 | 30.460 | −4.215 | −14.486 | 1.00 | 26.63 |
| ATOM | 11673 | N | ALA | 1730 | 29.526 | −2.181 | −14.321 | 1.00 | 26.96 |
| ATOM | 11674 | CA | ALA | 1730 | 28.165 | −2.694 | −14.435 | 1.00 | 26.38 |
| ATOM | 11675 | CB | ALA | 1730 | 27.243 | −1.938 | −13.486 | 1.00 | 25.85 |
| ATOM | 11676 | C | ALA | 1730 | 27.627 | −2.611 | −15.852 | 1.00 | 26.64 |
| ATOM | 11677 | O | ALA | 1730 | 28.155 | −1.886 | −16.694 | 1.00 | 27.21 |
| ATOM | 11678 | N | LYS | 1731 | 26.565 | −3.362 | −16.112 | 1.00 | 25.32 |
| ATOM | 11679 | CA | LYS | 1731 | 25.951 | −3.348 | −17.427 | 1.00 | 26.32 |
| ATOM | 11680 | CB | LYS | 1731 | 26.419 | −4.550 | −18.241 | 1.00 | 27.13 |
| ATOM | 11681 | CG | LYS | 1731 | 25.726 | −4.683 | −19.587 | 1.00 | 29.75 |
| ATOM | 11682 | CD | LYS | 1731 | 26.304 | −5.843 | −20.375 | 1.00 | 31.89 |
| ATOM | 11683 | CE | LYS | 1731 | 25.551 | −6.072 | −21.667 | 1.00 | 32.08 |
| ATOM | 11684 | NZ | LYS | 1731 | 26.136 | −7.215 | −22.417 | 1.00 | 34.11 |
| ATOM | 11685 | C | LYS | 1731 | 24.431 | −3.361 | −17.307 | 1.00 | 25.48 |
| ATOM | 11686 | O | LYS | 1731 | 23.868 | −4.094 | −16.498 | 1.00 | 24.67 |
| ATOM | 11687 | N | ASN | 1732 | 23.779 | −2.534 | −18.113 | 1.00 | 24.62 |
| ATOM | 11688 | CA | ASN | 1732 | 22.326 | −2.450 | −18.112 | 1.00 | 24.54 |
| ATOM | 11689 | CB | ASN | 1732 | 21.883 | −1.055 | −18.565 | 1.00 | 23.33 |
| ATOM | 11690 | CG | ASN | 1732 | 20.371 | −0.911 | −18.640 | 1.00 | 21.61 |
| ATOM | 11691 | OD1 | ASN | 1732 | 19.637 | −1.889 | −18.565 | 1.00 | 19.03 |
| ATOM | 11692 | ND2 | ASN | 1732 | 19.903 | 0.326 | −18.804 | 1.00 | 23.82 |
| ATOM | 11693 | C | ASN | 1732 | 21.785 | −3.508 | −19.068 | 1.00 | 25.76 |
| ATOM | 11694 | O | ASN | 1732 | 21.773 | −3.302 | −20.283 | 1.00 | 25.61 |
| ATOM | 11695 | N | PHE | 1733 | 21.344 | −4.637 | −18.520 | 1.00 | 25.01 |
| ATOM | 11696 | CA | PHE | 1733 | 20.811 | −5.719 | −19.337 | 1.00 | 25.29 |
| ATOM | 11697 | CB | PHE | 1733 | 20.880 | −7.049 | −18.582 | 1.00 | 26.19 |
| ATOM | 11698 | CG | PHE | 1733 | 22.279 | −7.524 | −18.325 | 1.00 | 25.90 |
| ATOM | 11699 | CD1 | PHE | 1733 | 22.973 | −7.108 | −17.195 | 1.00 | 26.87 |
| ATOM | 11700 | CD2 | PHE | 1733 | 22.922 | −8.353 | −19.237 | 1.00 | 26.66 |
| ATOM | 11701 | CE1 | PHE | 1733 | 24.292 | −7.510 | −16.975 | 1.00 | 25.54 |
| ATOM | 11702 | CE2 | PHE | 1733 | 24.242 | −8.761 | −19.029 | 1.00 | 26.56 |
| ATOM | 11703 | CZ | PHE | 1733 | 24.926 | −8.335 | −17.895 | 1.00 | 27.21 |
| ATOM | 11704 | C | PHE | 1733 | 19.380 | −5.471 | −19.807 | 1.00 | 26.55 |
| ATOM | 11705 | O | PHE | 1733 | 18.902 | −6.133 | −20.726 | 1.00 | 22.93 |
| ATOM | 11706 | N | LEU | 1734 | 18.696 | −4.521 | −19.178 | 1.00 | 27.50 |
| ATOM | 11707 | CA | LEU | 1734 | 17.330 | −4.212 | −19.569 | 1.00 | 29.76 |
| ATOM | 11708 | CB | LEU | 1734 | 16.605 | −3.451 | −18.461 | 1.00 | 28.65 |
| ATOM | 11709 | CG | LEU | 1734 | 15.196 | −2.972 | −18.829 | 1.00 | 27.48 |
| ATOM | 11710 | CD1 | LEU | 1734 | 14.348 | −4.147 | −19.304 | 1.00 | 25.62 |
| ATOM | 11711 | CD2 | LEU | 1734 | 14.566 | −2.305 | −17.625 | 1.00 | 23.90 |
| ATOM | 11712 | C | LEU | 1734 | 17.293 | −3.383 | −20.847 | 1.00 | 32.37 |
| ATOM | 11713 | O | LEU | 1734 | 16.613 | −3.748 | −21.804 | 1.00 | 31.12 |
| ATOM | 11714 | N | ALA | 1735 | 18.023 | −2.270 | −20.855 | 1.00 | 36.48 |
| ATOM | 11715 | CA | ALA | 1735 | 18.068 | −1.378 | −22.011 | 1.00 | 42.29 |
| ATOM | 11716 | CB | ALA | 1735 | 19.293 | −0.470 | −21.929 | 1.00 | 42.69 |
| ATOM | 11717 | C | ALA | 1735 | 18.100 | −2.186 | −23.297 | 1.00 | 46.05 |
| ATOM | 11718 | O | ALA | 1735 | 17.499 | −1.801 | −24.307 | 1.00 | 46.72 |
| ATOM | 11719 | N | GLU | 1736 | 18.797 | −3.317 | −23.239 | 1.00 | 49.05 |
| ATOM | 11720 | CA | GLU | 1736 | 18.923 | −4.225 | −24.374 | 1.00 | 51.86 |
| ATOM | 11721 | CB | GLU | 1736 | 19.548 | −5.551 | −23.913 | 1.00 | 53.55 |
| ATOM | 11722 | CG | GLU | 1736 | 20.814 | −5.411 | −23.072 | 1.00 | 55.23 |
| ATOM | 11723 | CD | GLU | 1736 | 22.018 | −4.944 | −23.873 | 1.00 | 57.29 |
| ATOM | 11724 | OE1 | GLU | 1736 | 21.940 | −3.866 | −24.505 | 1.00 | 58.69 |
| ATOM | 11725 | OE2 | GLU | 1736 | 23.046 | −5.657 | −23.869 | 1.00 | 57.04 |
| ATOM | 11726 | C | GLU | 1736 | 17.546 | −4.495 | −24.986 | 1.00 | 52.19 |
| ATOM | 11727 | O | GLU | 1736 | 17.355 | −4.351 | −26.195 | 1.00 | 53.04 |
| ATOM | 11728 | N | THR | 1737 | 16.594 | −4.876 | −24.134 | 1.00 | 51.41 |
| ATOM | 11729 | CA | THR | 1737 | 15.228 | −5.187 | −24.551 | 1.00 | 49.15 |
| ATOM | 11730 | CB | THR | 1737 | 14.969 | −6.699 | −24.442 | 1.00 | 50.52 |
| ATOM | 11731 | OG1 | THR | 1737 | 13.658 | −7.002 | −24.935 | 1.00 | 54.05 |
| ATOM | 11732 | CG2 | THR | 1737 | 15.082 | −7.146 | −22.993 | 1.00 | 51.48 |
| ATOM | 11733 | C | THR | 1737 | 14.202 | −4.449 | −23.685 | 1.00 | 46.27 |
| ATOM | 11734 | O | THR | 1737 | 14.491 | −3.381 | −23.149 | 1.00 | 47.44 |
| ATOM | 11735 | N | GLY | 1738 | 13.004 | −5.015 | −23.557 | 1.00 | 42.24 |
| ATOM | 11736 | CA | GLY | 1738 | 11.969 | −4.397 | −22.742 | 1.00 | 36.72 |
| ATOM | 11737 | C | GLY | 1738 | 11.296 | −5.412 | −21.831 | 1.00 | 33.77 |
| ATOM | 11738 | O | GLY | 1738 | 10.106 | −5.314 | −21.520 | 1.00 | 31.85 |
| ATOM | 11739 | N | ASP | 1739 | 12.076 | −6.395 | −21.398 | 1.00 | 31.02 |
| ATOM | 11740 | CA | ASP | 1739 | 11.587 | −7.461 | −20.535 | 1.00 | 29.23 |
| ATOM | 11741 | CB | ASP | 1739 | 11.418 | −8.744 | −21.358 | 1.00 | 32.58 |
| ATOM | 11742 | CG | ASP | 1739 | 10.678 | −9.832 | −20.609 | 1.00 | 35.23 |
| ATOM | 11743 | OD1 | ASP | 1739 | 11.103 | −10.195 | −19.493 | 1.00 | 35.59 |
| ATOM | 11744 | OD2 | ASP | 1739 | 9.667 | −10.332 | −21.147 | 1.00 | 38.35 |
| ATOM | 11745 | C | ASP | 1739 | 12.612 | −7.688 | −19.425 | 1.00 | 26.10 |
| ATOM | 11746 | O | ASP | 1739 | 13.748 | −8.092 | −19.691 | 1.00 | 24.19 |
| ATOM | 11747 | N | ILE | 1740 | 12.215 | −7.417 | −18.184 | 1.00 | 23.09 |
| ATOM | 11748 | CA | ILE | 1740 | 13.111 | −7.596 | −17.050 | 1.00 | 21.06 |
| ATOM | 11749 | CB | ILE | 1740 | 12.454 | −7.102 | −15.740 | 1.00 | 20.26 |
| ATOM | 11750 | CG2 | ILE | 1740 | 13.307 | −7.516 | −14.531 | 1.00 | 21.30 |
| ATOM | 11751 | CG1 | ILE | 1740 | 12.300 | −5.584 | −15.794 | 1.00 | 20.28 |
| ATOM | 11752 | CD1 | ILE | 1740 | 11.517 | −4.983 | −14.637 | 1.00 | 21.21 |
| ATOM | 11753 | C | ILE | 1740 | 13.545 | −9.049 | −16.896 | 1.00 | 19.53 |
| ATOM | 11754 | O | ILE | 1740 | 14.724 | −9.337 | −16.663 | 1.00 | 20.00 |
| ATOM | 11755 | N | ARG | 1741 | 12.600 | −9.971 | −17.030 | 1.00 | 18.98 |
| ATOM | 11756 | CA | ARG | 1741 | 12.937 | −11.384 | −16.911 | 1.00 | 19.96 |
| ATOM | 11757 | CB | ARG | 1741 | 11.668 | −12.236 | −16.974 | 1.00 | 19.87 |
| ATOM | 11758 | CG | ARG | 1741 | 10.849 | −12.142 | −15.696 | 1.00 | 21.32 |
| ATOM | 11759 | CD | ARG | 1741 | 9.513 | −12.853 | −15.810 | 1.00 | 23.47 |
| ATOM | 11760 | NE | ARG | 1741 | 8.776 | −12.794 | −14.549 | 1.00 | 25.80 |
| ATOM | 11761 | CZ | ARG | 1741 | 8.294 | −11.676 | −14.013 | 1.00 | 27.35 |
| ATOM | 11762 | NH1 | ARG | 1741 | 8.462 | −10.514 | −14.627 | 1.00 | 29.85 |
| ATOM | 11763 | NH2 | ARG | 1741 | 7.651 | −11.716 | −12.853 | 1.00 | 28.28 |
| ATOM | 11764 | C | ARG | 1741 | 13.936 | −11.809 | −17.984 | 1.00 | 20.67 |
| ATOM | 11765 | O | ARG | 1741 | 14.888 | −12.546 | −17.702 | 1.00 | 20.44 |
| ATOM | 11766 | N | ALA | 1742 | 13.733 | −11.333 | −19.209 | 1.00 | 18.88 |
| ATOM | 11767 | CA | ALA | 1742 | 14.642 | −11.663 | −20.299 | 1.00 | 19.67 |
| ATOM | 11768 | CB | ALA | 1742 | 14.100 | −11.119 | −21.617 | 1.00 | 17.58 |
| ATOM | 11769 | C | ALA | 1742 | 16.012 | −11.057 | −20.002 | 1.00 | 19.77 |
| ATOM | 11770 | O | ALA | 1742 | 17.040 | −11.601 | −20.391 | 1.00 | 21.64 |
| ATOM | 11771 | N | ALA | 1743 | 16.011 | −9.923 | −19.308 | 1.00 | 19.15 |
| ATOM | 11772 | CA | ALA | 1743 | 17.245 | −9.244 | −18.946 | 1.00 | 18.94 |
| ATOM | 11773 | CB | ALA | 1743 | 16.934 | −7.880 | −18.354 | 1.00 | 19.04 |
| ATOM | 11774 | C | ALA | 1743 | 18.006 | −10.095 | −17.932 | 1.00 | 19.62 |
| ATOM | 11775 | O | ALA | 1743 | 19.231 | −10.194 | −17.984 | 1.00 | 19.37 |
| ATOM | 11776 | N | VAL | 1744 | 17.268 | −10.706 | −17.010 | 1.00 | 20.08 |
| ATOM | 11777 | CA | VAL | 1744 | 17.861 | −11.560 | −15.992 | 1.00 | 19.96 |
| ATOM | 11778 | CB | VAL | 1744 | 16.791 | −12.000 | −14.961 | 1.00 | 21.45 |
| ATOM | 11779 | CG1 | VAL | 1744 | 17.314 | −13.143 | −14.099 | 1.00 | 19.83 |
| ATOM | 11780 | CG2 | VAL | 1744 | 16.429 | −10.812 | −14.071 | 1.00 | 18.35 |
| ATOM | 11781 | C | VAL | 1744 | 18.485 | −12.783 | −16.659 | 1.00 | 22.05 |
| ATOM | 11782 | O | VAL | 1744 | 19.602 | −13.197 | −16.326 | 1.00 | 21.12 |
| ATOM | 11783 | N | ARG | 1745 | 17.762 | −13.356 | −17.612 | 1.00 | 21.99 |
| ATOM | 11784 | CA | ARG | 1745 | 18.265 | −14.515 | −18.333 | 1.00 | 24.40 |
| ATOM | 11785 | CB | ARG | 1745 | 17.191 | −15.066 | −19.275 | 1.00 | 25.74 |
| ATOM | 11786 | CG | ARG | 1745 | 15.991 | −15.664 | −18.561 | 1.00 | 26.03 |
| ATOM | 11787 | CD | ARG | 1745 | 15.141 | −16.466 | −19.527 | 1.00 | 29.74 |
| ATOM | 11788 | NE | ARG | 1745 | 14.575 | −15.629 | −20.580 | 1.00 | 31.09 |
| ATOM | 11789 | CZ | ARG | 1745 | 13.454 | −14.924 | −20.456 | 1.00 | 33.26 |
| ATOM | 11790 | NH1 | ARG | 1745 | 12.767 | −14.957 | −19.320 | 1.00 | 31.03 |
| ATOM | 11791 | NH2 | ARG | 1745 | 13.024 | −14.183 | −21.470 | 1.00 | 33.72 |
| ATOM | 11792 | C | ARG | 1745 | 19.523 | −14.161 | −19.126 | 1.00 | 24.45 |
| ATOM | 11793 | O | ARG | 1745 | 20.420 | −14.994 | −19.283 | 1.00 | 23.74 |
| ATOM | 11794 | N | GLN | 1746 | 19.598 | −12.925 | −19.612 | 1.00 | 24.53 |
| ATOM | 11795 | CA | GLN | 1746 | 20.759 | −12.490 | −20.389 | 1.00 | 24.66 |
| ATOM | 11796 | CB | GLN | 1746 | 20.500 | −11.120 | −21.022 | 1.00 | 24.98 |
| ATOM | 11797 | CG | GLN | 1746 | 21.506 | −10.729 | −22.100 | 1.00 | 26.46 |
| ATOM | 11798 | CD | GLN | 1746 | 21.249 | −9.339 | −22.651 | 1.00 | 28.72 |
| ATOM | 11799 | OE1 | GLN | 1746 | 20.132 | −9.019 | −23.066 | 1.00 | 27.11 |
| ATOM | 11800 | NE2 | GLN | 1746 | 22.282 | −8.506 | −22.662 | 1.00 | 30.62 |
| ATOM | 11801 | C | GLN | 1746 | 21.984 | −12.410 | −19.491 | 1.00 | 24.23 |
| ATOM | 11802 | O | GLN | 1746 | 23.071 | −12.850 | −19.860 | 1.00 | 23.92 |
| ATOM | 11803 | N | TYR | 1747 | 21.800 | −11.831 | −18.311 | 1.00 | 23.27 |
| ATOM | 11804 | CA | TYR | 1747 | 22.883 | −11.693 | −17.352 | 1.00 | 22.18 |
| ATOM | 11805 | CB | TYR | 1747 | 22.363 | −10.949 | −16.114 | 1.00 | 21.72 |
| ATOM | 11806 | CG | TYR | 1747 | 23.266 | −10.978 | −14.901 | 1.00 | 20.80 |
| ATOM | 11807 | CD1 | TYR | 1747 | 24.581 | −10.525 | −14.969 | 1.00 | 20.83 |
| ATOM | 11808 | CE1 | TYR | 1747 | 25.406 | −10.540 | −13.844 | 1.00 | 21.19 |
| ATOM | 11809 | CD2 | TYR | 1747 | 22.794 | −11.446 | −13.677 | 1.00 | 20.71 |
| ATOM | 11810 | CE2 | TYR | 1747 | 23.610 | −11.463 | −12.546 | 1.00 | 21.91 |
| ATOM | 11811 | CZ | TYR | 1747 | 24.913 | −11.011 | −12.640 | 1.00 | 20.88 |
| ATOM | 11812 | OH | TYR | 1747 | 25.730 | −11.044 | −11.530 | 1.00 | 22.08 |
| ATOM | 11813 | C | TYR | 1747 | 23.417 | −13.079 | −16.987 | 1.00 | 21.20 |
| ATOM | 11814 | O | TYR | 1747 | 24.625 | −13.303 | −16.992 | 1.00 | 21.41 |
| ATOM | 11815 | N | MET | 1748 | 22.516 | −14.010 | −16.692 | 1.00 | 19.89 |
| ATOM | 11816 | CA | MET | 1748 | 22.915 | −15.370 | −16.327 | 1.00 | 22.82 |
| ATOM | 11817 | CB | MET | 1748 | 21.680 | −16.205 | −15.994 | 1.00 | 22.86 |
| ATOM | 11818 | CG | MET | 1748 | 20.883 | −15.673 | −14.814 | 1.00 | 24.78 |
| ATOM | 11819 | SD | MET | 1748 | 19.282 | −16.477 | −14.671 | 1.00 | 29.17 |
| ATOM | 11820 | CE | MET | 1748 | 19.786 | −18.014 | −13.979 | 1.00 | 26.45 |
| ATOM | 11821 | C | MET | 1748 | 23.692 | −16.047 | −17.452 | 1.00 | 22.62 |
| ATOM | 11822 | O | MET | 1748 | 24.653 | −16.786 | −17.213 | 1.00 | 21.81 |
| ATOM | 11823 | N | ALA | 1749 | 23.262 | −15.790 | −18.680 | 1.00 | 22.35 |
| ATOM | 11824 | CA | ALA | 1749 | 23.900 | −16.374 | −19.848 | 1.00 | 21.37 |
| ATOM | 11825 | CB | ALA | 1749 | 22.980 | −16.228 | −21.061 | 1.00 | 22.11 |
| ATOM | 11826 | C | ALA | 1749 | 25.268 | −15.766 | −20.148 | 1.00 | 20.89 |
| ATOM | 11827 | O | ALA | 1749 | 26.212 | −16.489 | −20.461 | 1.00 | 23.24 |
| ATOM | 11828 | N | GLU | 1750 | 25.390 | −14.446 | −20.053 | 1.00 | 21.41 |
| ATOM | 11829 | CA | GLU | 1750 | 26.671 | −13.808 | −20.352 | 1.00 | 21.76 |
| ATOM | 11830 | CB | GLU | 1750 | 26.497 | −12.300 | −20.528 | 1.00 | 23.39 |
| ATOM | 11831 | CG | GLU | 1750 | 25.501 | −11.917 | −21.613 | 1.00 | 25.73 |
| ATOM | 11832 | CD | GLU | 1750 | 25.685 | −10.499 | −22.093 | 1.00 | 25.79 |
| ATOM | 11833 | OE1 | GLU | 1750 | 26.438 | −9.746 | −21.442 | 1.00 | 25.87 |
| ATOM | 11834 | OE2 | GLU | 1750 | 25.070 | −10.130 | −23.123 | 1.00 | 28.21 |
| ATOM | 11835 | C | GLU | 1750 | 27.749 | −14.084 | −19.307 | 1.00 | 23.50 |
| ATOM | 11836 | O | GLU | 1750 | 28.942 | −14.115 | −19.627 | 1.00 | 21.76 |
| ATOM | 11837 | N | VAL | 1751 | 27.345 | −14.269 | −18.054 | 1.00 | 23.02 |
| ATOM | 11838 | CA | VAL | 1751 | 28.317 | −14.558 | −17.007 | 1.00 | 23.63 |
| ATOM | 11839 | CB | VAL | 1751 | 27.674 | −14.482 | −15.593 | 1.00 | 23.59 |
| ATOM | 11840 | CG1 | VAL | 1751 | 28.597 | −15.124 | −14.563 | 1.00 | 22.90 |
| ATOM | 11841 | CG2 | VAL | 1751 | 27.431 | −13.029 | −15.214 | 1.00 | 22.54 |
| ATOM | 11842 | C | VAL | 1751 | 28.893 | −15.952 | −17.233 | 1.00 | 24.14 |
| ATOM | 11843 | O | VAL | 1751 | 30.100 | −16.164 | −17.100 | 1.00 | 25.38 |
| ATOM | 11844 | N | GLU | 1752 | 28.028 | −16.895 | −17.589 | 1.00 | 24.05 |
| ATOM | 11845 | CA | GLU | 1752 | 28.459 | −18.264 | −17.834 | 1.00 | 26.81 |
| ATOM | 11846 | CB | GLU | 1752 | 27.247 | −19.196 | −17.907 | 1.00 | 28.09 |
| ATOM | 11847 | CG | GLU | 1752 | 27.611 | −20.655 | −18.151 | 1.00 | 30.83 |
| ATOM | 11848 | CD | GLU | 1752 | 26.452 | −21.589 | −17.904 | 1.00 | 30.97 |
| ATOM | 11849 | OE1 | GLU | 1752 | 26.551 | −22.775 | −18.279 | 1.00 | 32.52 |
| ATOM | 11850 | OE2 | GLU | 1752 | 25.439 | −21.143 | −17.328 | 1.00 | 31.48 |
| ATOM | 11851 | C | GLU | 1752 | 29.284 | −18.397 | −19.113 | 1.00 | 28.55 |
| ATOM | 11852 | O | GLU | 1752 | 30.237 | −19.173 | −19.164 | 1.00 | 29.30 |
| ATOM | 11853 | N | SER | 1753 | 28.914 | −17.646 | −20.146 | 1.00 | 28.43 |
| ATOM | 11854 | CA | SER | 1753 | 29.640 | −17.701 | −21.413 | 1.00 | 29.68 |
| ATOM | 11855 | CB | SER | 1753 | 28.832 | −17.023 | −22.521 | 1.00 | 30.24 |
| ATOM | 11856 | OG | SER | 1753 | 27.577 | −17.653 | −22.696 | 1.00 | 36.50 |
| ATOM | 11857 | C | SER | 1753 | 30.988 | −17.008 | −21.283 | 1.00 | 30.05 |
| ATOM | 11858 | O | SER | 1753 | 31.950 | −17.366 | −21.961 | 1.00 | 29.58 |
| ATOM | 11859 | N | GLY | 1754 | 31.047 | −16.013 | −20.404 | 1.00 | 28.51 |
| ATOM | 11860 | CA | GLY | 1754 | 32.276 | −15.269 | −20.213 | 1.00 | 27.36 |
| ATOM | 11861 | C | GLY | 1754 | 32.203 | −13.911 | −20.888 | 1.00 | 26.65 |
| ATOM | 11862 | O | GLY | 1754 | 33.136 | −13.113 | −20.801 | 1.00 | 26.59 |
| ATOM | 11863 | N | VAL | 1755 | 31.085 | −13.644 | −21.556 | 1.00 | 25.85 |
| ATOM | 11864 | CA | VAL | 1755 | 30.885 | −12.379 | −22.252 | 1.00 | 26.68 |
| ATOM | 11865 | CB | VAL | 1755 | 29.604 | −12.421 | −23.099 | 1.00 | 28.95 |
| ATOM | 11866 | CG1 | VAL | 1755 | 29.419 | −11.106 | −23.840 | 1.00 | 29.75 |
| ATOM | 11867 | CG2 | VAL | 1755 | 29.686 | −13.578 | −24.081 | 1.00 | 31.72 |
| ATOM | 11868 | C | VAL | 1755 | 30.807 | −11.205 | −21.286 | 1.00 | 26.82 |
| ATOM | 11869 | O | VAL | 1755 | 31.157 | −10.073 | −21.636 | 1.00 | 25.55 |
| ATOM | 11870 | N | TYR | 1756 | 30.346 | −11.480 | −20.068 | 1.00 | 25.63 |
| ATOM | 11871 | CA | TYR | 1756 | 30.233 | −10.455 | −19.031 | 1.00 | 25.13 |
| ATOM | 11872 | CB | TYR | 1756 | 28.764 | −10.150 | −18.707 | 1.00 | 25.56 |
| ATOM | 11873 | CG | TYR | 1756 | 28.618 | −9.068 | −17.657 | 1.00 | 24.46 |
| ATOM | 11874 | CD1 | TYR | 1756 | 28.867 | −7.734 | −17.974 | 1.00 | 24.19 |
| ATOM | 11875 | CE1 | TYR | 1756 | 28.852 | −6.745 | −16.998 | 1.00 | 25.87 |
| ATOM | 11876 | CD2 | TYR | 1756 | 28.336 | −9.388 | −16.327 | 1.00 | 24.07 |
| ATOM | 11877 | CE2 | TYR | 1756 | 28.319 | −8.406 | −15.338 | 1.00 | 24.15 |
| ATOM | 11878 | CZ | TYR | 1756 | 28.583 | −7.086 | −15.679 | 1.00 | 24.77 |
| ATOM | 11879 | OH | TYR | 1756 | 28.632 | −6.112 | −14.706 | 1.00 | 24.55 |
| ATOM | 11880 | C | TYR | 1756 | 30.931 | −10.927 | −17.761 | 1.00 | 25.03 |
| ATOM | 11881 | O | TYR | 1756 | 30.743 | −12.059 | −17.329 | 1.00 | 22.60 |
| ATOM | 11882 | N | PRO | 1757 | 31.748 | −10.056 | −17.145 | 1.00 | 27.09 |
| ATOM | 11883 | CD | PRO | 1757 | 32.331 | −10.263 | −15.807 | 1.00 | 26.60 |
| ATOM | 11884 | CA | PRO | 1757 | 32.016 | −8.698 | −17.622 | 1.00 | 28.05 |
| ATOM | 11885 | CB | PRO | 1757 | 32.530 | −7.995 | −16.370 | 1.00 | 29.31 |
| ATOM | 11886 | CG | PRO | 1757 | 33.281 | −9.082 | −15.679 | 1.00 | 29.30 |
| ATOM | 11887 | C | PRO | 1757 | 33.031 | −8.664 | −18.761 | 1.00 | 30.41 |
| ATOM | 11888 | O | PRO | 1757 | 33.900 | −9.529 | −18.855 | 1.00 | 30.25 |
| ATOM | 11889 | N | GLY | 1758 | 32.911 | −7.663 | −19.627 | 1.00 | 31.45 |
| ATOM | 11890 | CA | GLY | 1758 | 33.839 | −7.537 | −20.735 | 1.00 | 32.26 |
| ATOM | 11891 | C | GLY | 1758 | 35.007 | −6.657 | −20.339 | 1.00 | 33.47 |
| ATOM | 11892 | O | GLY | 1758 | 35.073 | −6.183 | −19.202 | 1.00 | 30.45 |
| ATOM | 11893 | N | GLU | 1759 | 35.932 | −6.427 | −21.265 | 1.00 | 34.20 |
| ATOM | 11894 | CA | GLU | 1759 | 37.088 | −5.589 | −20.966 | 1.00 | 36.05 |
| ATOM | 11895 | CB | GLU | 1759 | 38.020 | −5.500 | −22.179 | 1.00 | 37.61 |
| ATOM | 11896 | CG | GLU | 1759 | 39.230 | −4.611 | −21.932 | 1.00 | 39.60 |
| ATOM | 11897 | CD | GLU | 1759 | 40.075 | −5.093 | −20.765 | 1.00 | 40.84 |
| ATOM | 11898 | OE1 | GLU | 1759 | 40.868 | −4.288 | −20.231 | 1.00 | 43.08 |
| ATOM | 11899 | OE2 | GLU | 1759 | 39.949 | −6.278 | −20.390 | 1.00 | 40.65 |
| ATOM | 11900 | C | GLU | 1759 | 36.665 | −4.181 | −20.550 | 1.00 | 36.19 |
| ATOM | 11901 | O | GLU | 1759 | 37.293 | −3.560 | −19.693 | 1.00 | 36.76 |
| ATOM | 11902 | N | GLU | 1760 | 35.596 | −3.683 | −21.162 | 1.00 | 37.70 |
| ATOM | 11903 | CA | GLU | 1760 | 35.095 | −2.347 | −20.863 | 1.00 | 38.57 |
| ATOM | 11904 | CB | GLU | 1760 | 33.950 | −1.980 | −21.809 | 1.00 | 41.14 |
| ATOM | 11905 | CG | GLU | 1760 | 34.038 | −2.619 | −23.182 | 1.00 | 43.82 |
| ATOM | 11906 | CD | GLU | 1760 | 33.669 | −4.089 | −23.154 | 1.00 | 44.67 |
| ATOM | 11907 | OE1 | GLU | 1760 | 32.538 | −4.404 | −22.730 | 1.00 | 45.33 |
| ATOM | 11908 | OE2 | GLU | 1760 | 34.508 | −4.925 | −23.556 | 1.00 | 45.49 |
| ATOM | 11909 | C | GLU | 1760 | 34.598 | −2.255 | −19.426 | 1.00 | 37.43 |
| ATOM | 11910 | O | GLU | 1760 | 34.479 | −1.162 | −18.873 | 1.00 | 37.27 |
| ATOM | 11911 | N | HIS | 1761 | 34.306 | −3.411 | −18.835 | 1.00 | 36.62 |
| ATOM | 11912 | CA | HIS | 1761 | 33.809 | −3.487 | −17.462 | 1.00 | 36.41 |
| ATOM | 11913 | CB | HIS | 1761 | 32.650 | −4.485 | −17.373 | 1.00 | 34.33 |
| ATOM | 11914 | CG | HIS | 1761 | 31.541 | −4.218 | −18.343 | 1.00 | 33.22 |
| ATOM | 11915 | CD2 | HIS | 1761 | 31.033 | −4.973 | −19.346 | 1.00 | 31.29 |
| ATOM | 11916 | ND1 | HIS | 1761 | 30.806 | −3.053 | −18.329 | 1.00 | 32.96 |
| ATOM | 11917 | CE1 | HIS | 1761 | 29.892 | −3.101 | −19.281 | 1.00 | 32.22 |
| ATOM | 11918 | NE2 | HIS | 1761 | 30.008 | −4.255 | −19.912 | 1.00 | 32.69 |
| ATOM | 11919 | C | HIS | 1761 | 34.905 | −3.941 | −16.504 | 1.00 | 37.42 |
| ATOM | 11920 | O | HIS | 1761 | 34.620 | −4.320 | −15.369 | 1.00 | 38.41 |
| ATOM | 11921 | N | SER | 1762 | 36.153 | −3.898 | −16.960 | 1.00 | 37.25 |
| ATOM | 11922 | CA | SER | 1762 | 37.276 | −4.338 | −16.144 | 1.00 | 37.81 |
| ATOM | 11923 | CB | SER | 1762 | 37.960 | −5.527 | −16.819 | 1.00 | 37.82 |
| ATOM | 11924 | OG | SER | 1762 | 37.022 | −6.539 | −17.142 | 1.00 | 36.90 |
| ATOM | 11925 | C | SER | 1762 | 38.309 | −3.245 | −15.884 | 1.00 | 39.62 |
| ATOM | 11926 | O | SER | 1762 | 38.410 | −2.277 | −16.642 | 1.00 | 37.84 |
| ATOM | 11927 | N | PHE | 1763 | 39.069 | −3.402 | −14.802 | 1.00 | 40.54 |
| ATOM | 11928 | CA | PHE | 1763 | 40.112 | −2.441 | −14.458 | 1.00 | 43.38 |
| ATOM | 11929 | CB | PHE | 1763 | 39.929 | −1.897 | −13.037 | 1.00 | 44.18 |
| ATOM | 11930 | CG | PHE | 1763 | 38.661 | −1.112 | −12.840 | 1.00 | 45.64 |
| ATOM | 11931 | CD1 | PHE | 1763 | 37.477 | −1.752 | −12.485 | 1.00 | 46.71 |
| ATOM | 11932 | CD2 | PHE | 1763 | 38.648 | 0.269 | −13.014 | 1.00 | 46.22 |
| ATOM | 11933 | CE1 | PHE | 1763 | 36.297 | −1.028 | −12.305 | 1.00 | 46.58 |
| ATOM | 11934 | CE2 | PHE | 1763 | 37.475 | 1.004 | −12.838 | 1.00 | 46.28 |
| ATOM | 11935 | CZ | PHE | 1763 | 36.297 | 0.353 | −12.482 | 1.00 | 46.49 |
| ATOM | 11936 | C | PHE | 1763 | 41.475 | −3.110 | −14.567 | 1.00 | 45.31 |
| ATOM | 11937 | O | PHE | 1763 | 41.568 | −4.325 | −14.742 | 1.00 | 45.15 |
| ATOM | 11938 | N | HIS | 1764 | 42.531 | −2.309 | −14.464 | 1.00 | 47.11 |
| ATOM | 11939 | CA | HIS | 1764 | 43.897 | −2.814 | −14.551 | 1.00 | 48.73 |
| ATOM | 11940 | CB | HIS | 1764 | 44.368 | −2.804 | −16.007 | 1.00 | 48.11 |
| ATOM | 11941 | CG | HIS | 1764 | 43.714 | −3.847 | −16.858 | 1.00 | 47.92 |
| ATOM | 11942 | CD2 | HIS | 1764 | 42.833 | −3.742 | −17.881 | 1.00 | 47.14 |
| ATOM | 11943 | ND1 | HIS | 1764 | 43.935 | −5.196 | −16.681 | 1.00 | 48.69 |
| ATOM | 11944 | CE1 | HIS | 1764 | 43.218 | −5.877 | −17.557 | 1.00 | 47.18 |
| ATOM | 11945 | NE2 | HIS | 1764 | 42.541 | −5.018 | −18.297 | 1.00 | 46.67 |
| ATOM | 11946 | C | HIS | 1764 | 44.848 | −1.989 | −13.694 | 1.00 | 49.73 |
| ATOM | 11947 | O | HIS | 1764 | 45.534 | −2.589 | −12.839 | 1.00 | 50.49 |
| ATOM | 11948 | OXT | HIS | 1764 | 44.896 | −0.757 | −13.887 | 1.00 | 51.77 |
| ATOM | 11949 | C1 | KPL | 1765 | 27.748 | −4.209 | −4.469 | 1.00 | 37.13 |
| ATOM | 11950 | C2 | KPL | 1765 | 27.949 | −5.559 | −3.746 | 1.00 | 36.67 |
| ATOM | 11951 | C3 | KPL | 1765 | 27.104 | −6.630 | −4.447 | 1.00 | 36.77 |
| ATOM | 11952 | C4 | KPL | 1765 | 29.432 | −5.968 | −3.834 | 1.00 | 38.31 |
| ATOM | 11953 | O1 | KPL | 1765 | 30.255 | −4.985 | −3.190 | 1.00 | 40.50 |
| ATOM | 11954 | C5 | KPL | 1765 | 27.511 | −5.432 | −2.265 | 1.00 | 36.08 |
| ATOM | 11955 | O2 | KPL | 1765 | 28.306 | −5.650 | −1.372 | 1.00 | 36.16 |
| ATOM | 11956 | C6 | KPL | 1765 | 26.106 | −5.037 | −1.885 | 1.00 | 33.38 |
| ATOM | 11957 | O3 | KPL | 1765 | 25.273 | −4.814 | −2.736 | 1.00 | 35.07 |
| ATOM | 11958 | O4 | KPL | 1765 | 25.770 | −4.927 | −0.590 | 1.00 | 32.90 |
| ATOM | 11959 | CB | MET | 1801 | 10.432 | −42.440 | 39.264 | 1.00 | 62.99 |
| ATOM | 11960 | CG | MET | 1801 | 11.787 | −41.787 | 39.475 | 1.00 | 64.83 |
| ATOM | 11961 | SD | MET | 1801 | 12.950 | −42.846 | 40.338 | 1.00 | 67.79 |
| ATOM | 11962 | CE | MET | 1801 | 12.685 | −42.311 | 42.031 | 1.00 | 67.74 |
| ATOM | 11963 | C | MET | 1801 | 8.891 | −40.780 | 40.307 | 1.00 | 59.13 |
| ATOM | 11964 | O | MET | 1801 | 7.710 | −40.737 | 40.655 | 1.00 | 59.38 |
| ATOM | 11965 | N | MET | 1801 | 9.705 | −40.456 | 37.968 | 1.00 | 61.53 |
| ATOM | 11966 | CA | MET | 1801 | 9.294 | −41.449 | 38.998 | 1.00 | 60.88 |
| ATOM | 11967 | N | LYS | 1802 | 9.874 | −40.256 | 41.033 | 1.00 | 56.22 |
| ATOM | 11968 | CA | LYS | 1802 | 9.599 | −39.612 | 42.312 | 1.00 | 53.19 |
| ATOM | 11969 | CB | LYS | 1802 | 9.801 | −40.624 | 43.447 | 1.00 | 54.60 |
| ATOM | 11970 | CG | LYS | 1802 | 9.046 | −41.941 | 43.283 | 1.00 | 55.72 |
| ATOM | 11971 | CD | LYS | 1802 | 7.557 | −41.813 | 43.598 | 1.00 | 56.31 |
| ATOM | 11972 | CE | LYS | 1802 | 7.306 | −41.678 | 45.099 | 1.00 | 56.54 |
| ATOM | 11973 | NZ | LYS | 1802 | 5.851 | −41.687 | 45.436 | 1.00 | 55.62 |
| ATOM | 11974 | C | LYS | 1802 | 10.437 | −38.361 | 42.596 | 1.00 | 49.88 |
| ATOM | 11975 | O | LYS | 1802 | 10.760 | −38.088 | 43.759 | 1.00 | 50.44 |
| ATOM | 11976 | N | PRO | 1803 | 10.811 | −37.590 | 41.560 | 1.00 | 45.52 |
| ATOM | 11977 | CD | PRO | 1803 | 11.320 | −36.225 | 41.816 | 1.00 | 44.97 |
| ATOM | 11978 | CA | PRO | 1803 | 10.530 | −37.761 | 40.132 | 1.00 | 41.04 |
| ATOM | 11979 | CB | PRO | 1803 | 10.376 | −36.334 | 39.651 | 1.00 | 42.80 |
| ATOM | 11980 | CG | PRO | 1803 | 11.465 | −35.650 | 40.414 | 1.00 | 43.86 |
| ATOM | 11981 | C | PRO | 1803 | 11.674 | −38.481 | 39.414 | 1.00 | 36.05 |
| ATOM | 11982 | O | PRO | 1803 | 12.591 | −38.991 | 40.056 | 1.00 | 34.80 |
| ATOM | 11983 | N | THR | 1804 | 11.621 | −38.511 | 38.084 | 1.00 | 30.62 |
| ATOM | 11984 | CA | THR | 1804 | 12.666 | −39.155 | 37.283 | 1.00 | 26.97 |
| ATOM | 11985 | CB | THR | 1804 | 12.172 | −39.435 | 35.853 | 1.00 | 25.88 |
| ATOM | 11986 | OG1 | THR | 1804 | 11.046 | −40.323 | 35.902 | 1.00 | 24.56 |
| ATOM | 11987 | CG2 | THR | 1804 | 13.276 | −40.073 | 35.021 | 1.00 | 22.12 |
| ATOM | 11988 | C | THR | 1804 | 13.880 | −38.232 | 37.215 | 1.00 | 24.43 |
| ATOM | 11989 | O | THR | 1804 | 13.752 | −37.056 | 36.888 | 1.00 | 22.49 |
| ATOM | 11990 | N | THR | 1805 | 15.055 | −38.769 | 37.524 | 1.00 | 23.84 |
| ATOM | 11991 | CA | THR | 1805 | 16.278 | −37.973 | 37.516 | 1.00 | 22.21 |
| ATOM | 11992 | CB | THR | 1805 | 16.829 | −37.800 | 38.940 | 1.00 | 23.39 |
| ATOM | 11993 | OG1 | THR | 1805 | 17.286 | −39.072 | 39.423 | 1.00 | 25.01 |
| ATOM | 11994 | CG2 | THR | 1805 | 15.756 | −37.267 | 39.866 | 1.00 | 23.36 |
| ATOM | 11995 | C | THR | 1805 | 17.379 | −38.614 | 36.682 | 1.00 | 21.44 |
| ATOM | 11996 | O | THR | 1805 | 17.268 | −39.761 | 36.247 | 1.00 | 19.91 |
| ATOM | 11997 | N | ILE | 1806 | 18.453 | −37.863 | 36.471 | 1.00 | 19.22 |
| ATOM | 11998 | CA | ILE | 1806 | 19.574 | −38.376 | 35.706 | 1.00 | 20.35 |
| ATOM | 11999 | CB | ILE | 1806 | 20.696 | −37.329 | 35.599 | 1.00 | 22.61 |
| ATOM | 12000 | CG2 | ILE | 1806 | 21.806 | −37.846 | 34.689 | 1.00 | 23.50 |
| ATOM | 12001 | CG1 | ILE | 1806 | 20.133 | −36.030 | 35.016 | 1.00 | 24.71 |
| ATOM | 12002 | CD1 | ILE | 1806 | 21.079 | −34.859 | 35.129 | 1.00 | 27.86 |
| ATOM | 12003 | C | ILE | 1806 | 20.108 | −39.623 | 36.415 | 1.00 | 19.98 |
| ATOM | 12004 | O | ILE | 1806 | 20.612 | −40.551 | 35.780 | 1.00 | 17.26 |
| ATOM | 12005 | N | SER | 1807 | 19.979 | −39.652 | 37.736 | 1.00 | 19.88 |
| ATOM | 12006 | CA | SER | 1807 | 20.455 | −40.796 | 38.505 | 1.00 | 20.30 |
| ATOM | 12007 | CB | SER | 1807 | 20.201 | −40.578 | 40.000 | 1.00 | 22.82 |
| ATOM | 12008 | OG | SER | 1807 | 20.872 | −39.418 | 40.459 | 1.00 | 29.05 |
| ATOM | 12009 | C | SER | 1807 | 19.777 | −42.090 | 38.062 | 1.00 | 19.29 |
| ATOM | 12010 | O | SER | 1807 | 20.408 | −43.148 | 38.010 | 1.00 | 18.78 |
| ATOM | 12011 | N | LEU | 1808 | 18.491 | −42.002 | 37.741 | 1.00 | 19.36 |
| ATOM | 12012 | CA | LEU | 1808 | 17.731 | −43.170 | 37.317 | 1.00 | 19.70 |
| ATOM | 12013 | CB | LEU | 1808 | 16.248 | −42.828 | 37.204 | 1.00 | 21.62 |
| ATOM | 12014 | CG | LEU | 1808 | 15.233 | −43.791 | 37.821 | 1.00 | 26.64 |
| ATOM | 12015 | CD1 | LEU | 1808 | 13.896 | −43.565 | 37.130 | 1.00 | 27.41 |
| ATOM | 12016 | CD2 | LEU | 1808 | 15.658 | −45.247 | 37.649 | 1.00 | 26.23 |
| ATOM | 12017 | C | LEU | 1808 | 18.218 | −43.719 | 35.984 | 1.00 | 17.67 |
| ATOM | 12018 | O | LEU | 1808 | 18.294 | −44.934 | 35.797 | 1.00 | 16.92 |
| ATOM | 12019 | N | LEU | 1809 | 18.528 | −42.831 | 35.047 | 1.00 | 16.05 |
| ATOM | 12020 | CA | LEU | 1809 | 19.006 | −43.273 | 33.740 | 1.00 | 15.75 |
| ATOM | 12021 | CB | LEU | 1809 | 19.110 | −42.089 | 32.772 | 1.00 | 13.68 |
| ATOM | 12022 | CG | LEU | 1809 | 17.811 | −41.302 | 32.527 | 1.00 | 16.26 |
| ATOM | 12023 | CD1 | LEU | 1809 | 18.026 | −40.320 | 31.380 | 1.00 | 11.13 |
| ATOM | 12024 | CD2 | LEU | 1809 | 16.672 | −42.238 | 32.202 | 1.00 | 15.56 |
| ATOM | 12025 | C | LEU | 1809 | 20.363 | −43.936 | 33.900 | 1.00 | 16.76 |
| ATOM | 12026 | O | LEU | 1809 | 20.643 | −44.952 | 33.267 | 1.00 | 17.33 |
| ATOM | 12027 | N | GLN | 1810 | 21.207 | −43.363 | 34.752 | 1.00 | 18.12 |
| ATOM | 12028 | CA | GLN | 1810 | 22.526 | −43.943 | 34.978 | 1.00 | 19.74 |
| ATOM | 12029 | CB | GLN | 1810 | 23.325 | −43.078 | 35.957 | 1.00 | 22.02 |
| ATOM | 12030 | CG | GLN | 1810 | 24.818 | −43.410 | 36.021 | 1.00 | 24.70 |
| ATOM | 12031 | CD | GLN | 1810 | 25.527 | −43.270 | 34.676 | 1.00 | 24.85 |
| ATOM | 12032 | OE1 | GLN | 1810 | 25.549 | −44.197 | 33.869 | 1.00 | 26.62 |
| ATOM | 12033 | NE2 | GLN | 1810 | 26.100 | −42.101 | 34.430 | 1.00 | 25.00 |
| ATOM | 12034 | C | GLN | 1810 | 22.345 | −45.363 | 35.520 | 1.00 | 20.60 |
| ATOM | 12035 | O | GLN | 1810 | 23.081 | −46.275 | 35.142 | 1.00 | 19.97 |
| ATOM | 12036 | N | LYS | 1811 | 21.356 | −45.553 | 36.391 | 1.00 | 20.73 |
| ATOM | 12037 | CA | LYS | 1811 | 21.088 | −46.881 | 36.952 | 1.00 | 22.94 |
| ATOM | 12038 | CB | LYS | 1811 | 19.960 | −46.825 | 37.989 | 1.00 | 25.60 |
| ATOM | 12039 | CG | LYS | 1811 | 19.688 | −48.183 | 38.647 | 1.00 | 30.12 |
| ATOM | 12040 | CD | LYS | 1811 | 18.259 | −48.325 | 39.172 | 1.00 | 34.97 |
| ATOM | 12041 | CE | LYS | 1811 | 17.969 | −47.428 | 40.368 | 1.00 | 37.63 |
| ATOM | 12042 | NZ | LYS | 1811 | 16.572 | −47.640 | 40.868 | 1.00 | 38.97 |
| ATOM | 12043 | C | LYS | 1811 | 20.677 | −47.820 | 35.822 | 1.00 | 22.37 |
| ATOM | 12044 | O | LYS | 1811 | 21.130 | −48.965 | 35.750 | 1.00 | 23.23 |
| ATOM | 12045 | N | TYR | 1812 | 19.803 | −47.327 | 34.948 | 1.00 | 20.19 |
| ATOM | 12046 | CA | TYR | 1812 | 19.328 | −48.108 | 33.806 | 1.00 | 19.41 |
| ATOM | 12047 | CB | TYR | 1812 | 18.394 | −47.252 | 32.939 | 1.00 | 20.86 |
| ATOM | 12048 | CG | TYR | 1812 | 17.008 | −47.048 | 33.529 | 1.00 | 23.68 |
| ATOM | 12049 | CD1 | TYR | 1812 | 16.100 | −46.165 | 32.941 | 1.00 | 25.01 |
| ATOM | 12050 | CE1 | TYR | 1812 | 14.810 | −46.011 | 33.456 | 1.00 | 28.32 |
| ATOM | 12051 | CD2 | TYR | 1812 | 16.590 | −47.769 | 34.650 | 1.00 | 25.69 |
| ATOM | 12052 | CE2 | TYR | 1812 | 15.307 | −47.623 | 35.169 | 1.00 | 26.94 |
| ATOM | 12053 | CZ | TYR | 1812 | 14.424 | −46.748 | 34.568 | 1.00 | 27.83 |
| ATOM | 12054 | OH | TYR | 1812 | 13.147 | −46.626 | 35.067 | 1.00 | 33.55 |
| ATOM | 12055 | C | TYR | 1812 | 20.488 | −48.620 | 32.960 | 1.00 | 19.49 |
| ATOM | 12056 | O | TYR | 1812 | 20.478 | −49.768 | 32.508 | 1.00 | 18.51 |
| ATOM | 12057 | N | LYS | 1813 | 21.486 | −47.768 | 32.739 | 1.00 | 18.22 |
| ATOM | 12058 | CA | LYS | 1813 | 22.641 | −48.177 | 31.950 | 1.00 | 18.58 |
| ATOM | 12059 | CB | LYS | 1813 | 23.577 | −46.993 | 31.679 | 1.00 | 17.18 |
| ATOM | 12060 | CG | LYS | 1813 | 24.847 | −47.399 | 30.924 | 1.00 | 14.61 |
| ATOM | 12061 | CD | LYS | 1813 | 25.545 | −46.214 | 30.288 | 1.00 | 14.45 |
| ATOM | 12062 | CE | LYS | 1813 | 26.783 | −46.661 | 29.524 | 1.00 | 23.10 |
| ATOM | 12063 | NZ | LYS | 1813 | 26.884 | −45.976 | 28.193 | 1.00 | 12.51 |
| ATOM | 12064 | C | LYS | 1813 | 23.398 | −49.284 | 32.688 | 1.00 | 19.88 |
| ATOM | 12065 | O | LYS | 1813 | 23.840 | −50.247 | 32.076 | 1.00 | 19.85 |
| ATOM | 12066 | N | GLN | 1814 | 23.541 | −49.144 | 34.001 | 1.00 | 23.03 |
| ATOM | 12067 | CA | GLN | 1814 | 24.238 | −50.156 | 34.785 | 1.00 | 26.55 |
| ATOM | 12068 | CB | GLN | 1814 | 24.399 | −49.695 | 36.229 | 1.00 | 30.23 |
| ATOM | 12069 | CG | GLN | 1814 | 25.271 | −48.461 | 36.380 | 1.00 | 36.91 |
| ATOM | 12070 | CD | GLN | 1814 | 25.444 | −48.052 | 37.827 | 1.00 | 40.35 |
| ATOM | 12071 | OE1 | GLN | 1814 | 25.895 | −48.843 | 38.655 | 1.00 | 43.65 |
| ATOM | 12072 | NE2 | GLN | 1814 | 25.087 | −46.811 | 38.142 | 1.00 | 43.19 |
| ATOM | 12073 | C | GLN | 1814 | 23.481 | −51.480 | 34.745 | 1.00 | 26.92 |
| ATOM | 12074 | O | GLN | 1814 | 24.086 | −52.553 | 34.697 | 1.00 | 29.93 |
| ATOM | 12075 | N | GLU | 1815 | 22.155 | −51.402 | 34.756 | 1.00 | 26.78 |
| ATOM | 12076 | CA | GLU | 1815 | 21.313 | −52.595 | 34.720 | 1.00 | 26.97 |
| ATOM | 12077 | CB | GLU | 1815 | 19.947 | −52.290 | 35.342 | 1.00 | 29.31 |
| ATOM | 12078 | CG | GLU | 1815 | 20.023 | −51.678 | 36.735 | 1.00 | 35.92 |
| ATOM | 12079 | CD | GLU | 1815 | 18.652 | −51.375 | 37.325 | 1.00 | 38.20 |
| ATOM | 12080 | OE1 | GLU | 1815 | 17.878 | −50.624 | 36.694 | 1.00 | 40.52 |
| ATOM | 12081 | OE2 | GLU | 1815 | 18.352 | −51.886 | 38.424 | 1.00 | 42.17 |
| ATOM | 12082 | C | GLU | 1815 | 21.118 | −53.075 | 33.285 | 1.00 | 24.86 |
| ATOM | 12083 | O | GLU | 1815 | 20.503 | −54.113 | 33.048 | 1.00 | 24.50 |
| ATOM | 12084 | N | LYS | 1816 | 21.651 | −52.318 | 32.331 | 1.00 | 23.60 |
| ATOM | 12085 | CA | LYS | 1816 | 21.516 | −52.653 | 30.920 | 1.00 | 24.10 |
| ATOM | 12086 | CB | LYS | 1816 | 22.168 | −54.012 | 30.619 | 1.00 | 27.06 |
| ATOM | 12087 | CG | LYS | 1816 | 23.690 | −53.956 | 30.494 | 1.00 | 29.88 |
| ATOM | 12088 | CD | LYS | 1816 | 24.106 | −53.130 | 29.280 | 1.00 | 33.12 |
| ATOM | 12089 | CE | LYS | 1816 | 25.622 | −52.924 | 29.206 | 1.00 | 34.77 |
| ATOM | 12090 | NZ | LYS | 1816 | 26.378 | −54.205 | 29.060 | 1.00 | 36.10 |
| ATOM | 12091 | C | LYS | 1816 | 20.048 | −52.658 | 30.491 | 1.00 | 23.58 |
| ATOM | 12092 | O | LYS | 1816 | 19.635 | −53.456 | 29.650 | 1.00 | 22.90 |
| ATOM | 12093 | N | LYS | 1817 | 19.258 | −51.763 | 31.081 | 1.00 | 22.10 |
| ATOM | 12094 | CA | LYS | 1817 | 17.847 | −51.649 | 30.730 | 1.00 | 19.64 |
| ATOM | 12095 | CB | LYS | 1817 | 16.982 | −51.432 | 31.973 | 1.00 | 23.35 |
| ATOM | 12096 | CG | LYS | 1817 | 15.498 | −51.274 | 31.636 | 1.00 | 26.44 |
| ATOM | 12097 | CD | LYS | 1817 | 14.682 | −50.726 | 32.801 | 1.00 | 30.23 |
| ATOM | 12098 | CE | LYS | 1817 | 14.649 | −51.683 | 33.980 | 1.00 | 33.21 |
| ATOM | 12099 | NZ | LYS | 1817 | 13.841 | −51.115 | 35.101 | 1.00 | 36.99 |
| ATOM | 12100 | C | LYS | 1817 | 17.655 | −50.463 | 29.790 | 1.00 | 19.22 |
| ATOM | 12101 | O | LYS | 1817 | 17.761 | −49.311 | 30.212 | 1.00 | 15.14 |
| ATOM | 12102 | N | ARG | 1818 | 17.373 | −50.756 | 28.523 | 1.00 | 17.56 |
| ATOM | 12103 | CA | ARG | 1818 | 17.165 | −49.723 | 27.515 | 1.00 | 17.68 |
| ATOM | 12104 | CB | ARG | 1818 | 17.122 | −50.354 | 26.121 | 1.00 | 17.01 |
| ATOM | 12105 | CG | ARG | 1818 | 18.516 | −50.558 | 25.563 | 1.00 | 20.64 |
| ATOM | 12106 | CD | ARG | 1818 | 18.534 | −51.433 | 24.331 | 1.00 | 26.16 |
| ATOM | 12107 | NE | ARG | 1818 | 18.303 | −52.836 | 24.671 | 1.00 | 27.97 |
| ATOM | 12108 | CZ | ARG | 1818 | 18.504 | −53.847 | 23.833 | 1.00 | 30.30 |
| ATOM | 12109 | NH1 | ARG | 1818 | 18.942 | −53.611 | 22.602 | 1.00 | 31.08 |
| ATOM | 12110 | NH2 | ARG | 1818 | 18.266 | −55.091 | 24.223 | 1.00 | 30.79 |
| ATOM | 12111 | C | ARG | 1818 | 15.898 | −48.939 | 27.800 | 1.00 | 16.78 |
| ATOM | 12112 | O | ARG | 1818 | 14.858 | −49.522 | 28.107 | 1.00 | 17.29 |
| ATOM | 12113 | N | PHE | 1819 | 15.986 | −47.615 | 27.695 | 1.00 | 15.71 |
| ATOM | 12114 | CA | PHE | 1819 | 14.849 | −46.764 | 28.005 | 1.00 | 13.45 |
| ATOM | 12115 | CB | PHE | 1819 | 15.167 | −45.940 | 29.258 | 1.00 | 12.38 |
| ATOM | 12116 | CG | PHE | 1819 | 16.395 | −45.082 | 29.135 | 1.00 | 10.54 |
| ATOM | 12117 | CD1 | PHE | 1819 | 16.302 | −43.780 | 28.653 | 1.00 | 10.46 |
| ATOM | 12118 | CD2 | PHE | 1819 | 17.648 | −45.576 | 29.494 | 1.00 | 11.28 |
| ATOM | 12119 | CE1 | PHE | 1819 | 17.434 | −42.980 | 28.540 | 1.00 | 10.02 |
| ATOM | 12120 | CE2 | PHE | 1819 | 18.790 | −44.782 | 29.384 | 1.00 | 12.19 |
| ATOM | 12121 | CZ | PHE | 1819 | 18.684 | −43.484 | 28.903 | 1.00 | 10.00 |
| ATOM | 12122 | C | PHE | 1819 | 14.405 | −45.848 | 26.877 | 1.00 | 12.26 |
| ATOM | 12123 | O | PHE | 1819 | 15.214 | −45.404 | 26.057 | 1.00 | 11.06 |
| ATOM | 12124 | N | ALA | 1820 | 13.105 | −45.572 | 26.850 | 1.00 | 11.30 |
| ATOM | 12125 | CA | ALA | 1820 | 12.525 | −44.715 | 25.833 | 1.00 | 11.83 |
| ATOM | 12126 | CB | ALA | 1820 | 11.114 | −45.203 | 25.470 | 1.00 | 10.93 |
| ATOM | 12127 | C | ALA | 1820 | 12.464 | −43.256 | 26.262 | 1.00 | 11.52 |
| ATOM | 12128 | O | ALA | 1820 | 12.287 | −42.945 | 27.441 | 1.00 | 13.43 |
| ATOM | 12129 | N | THR | 1821 | 12.598 | −42.372 | 25.277 | 1.00 | 11.00 |
| ATOM | 12130 | CA | THR | 1821 | 12.552 | −40.928 | 25.481 | 1.00 | 11.82 |
| ATOM | 12131 | CB | THR | 1821 | 13.973 | −40.321 | 25.403 | 1.00 | 11.47 |
| ATOM | 12132 | OG1 | THR | 1821 | 14.756 | −40.809 | 26.505 | 1.00 | 15.36 |
| ATOM | 12133 | CG2 | THR | 1821 | 13.913 | −38.816 | 25.451 | 1.00 | 23.24 |
| ATOM | 12134 | C | THR | 1821 | 11.706 | −40.398 | 24.331 | 1.00 | 9.33 |
| ATOM | 12135 | O | THR | 1821 | 11.554 | −41.075 | 23.312 | 1.00 | 12.52 |
| ATOM | 12136 | N | ILE | 1822 | 11.150 | −39.201 | 24.465 | 1.00 | 10.58 |
| ATOM | 12137 | CA | ILE | 1822 | 10.315 | −38.690 | 23.385 | 1.00 | 9.32 |
| ATOM | 12138 | CB | ILE | 1822 | 8.892 | −39.296 | 23.504 | 1.00 | 10.75 |
| ATOM | 12139 | CG2 | ILE | 1822 | 8.162 | −38.672 | 24.716 | 1.00 | 10.54 |
| ATOM | 12140 | CG1 | ILE | 1822 | 8.102 | −39.050 | 22.222 | 1.00 | 13.24 |
| ATOM | 12141 | CD1 | ILE | 1822 | 6.801 | −39.836 | 22.165 | 1.00 | 14.23 |
| ATOM | 12142 | C | ILE | 1822 | 10.237 | −37.169 | 23.415 | 1.00 | 12.04 |
| ATOM | 12143 | O | ILE | 1822 | 10.473 | −36.552 | 24.456 | 1.00 | 9.11 |
| ATOM | 12144 | N | THR | 1823 | 9.926 | −36.565 | 22.269 | 1.00 | 10.88 |
| ATOM | 12145 | CA | THR | 1823 | 9.778 | −35.119 | 22.212 | 1.00 | 12.73 |
| ATOM | 12146 | CB | THR | 1823 | 9.990 | −34.570 | 20.766 | 1.00 | 11.54 |
| ATOM | 12147 | OG1 | THR | 1823 | 9.000 | −35.126 | 19.889 | 1.00 | 12.04 |
| ATOM | 12148 | CG2 | THR | 1823 | 11.393 | −34.936 | 20.259 | 1.00 | 14.24 |
| ATOM | 12149 | C | THR | 1823 | 8.359 | −34.785 | 22.681 | 1.00 | 12.09 |
| ATOM | 12150 | O | THR | 1823 | 7.436 | −35.591 | 22.546 | 1.00 | 11.41 |
| ATOM | 12151 | N | ALA | 1824 | 8.203 | −33.599 | 23.257 | 1.00 | 10.84 |
| ATOM | 12152 | CA | ALA | 1824 | 6.912 | −33.134 | 23.756 | 1.00 | 10.40 |
| ATOM | 12153 | CB | ALA | 1824 | 6.659 | −33.673 | 25.173 | 1.00 | 9.05 |
| ATOM | 12154 | C | ALA | 1824 | 6.973 | −31.604 | 23.776 | 1.00 | 10.44 |
| ATOM | 12155 | O | ALA | 1824 | 8.015 | −31.023 | 24.087 | 1.00 | 9.73 |
| ATOM | 12156 | N | TYR | 1825 | 5.867 | −30.947 | 23.445 | 1.00 | 9.09 |
| ATOM | 12157 | CA | TYR | 1825 | 5.864 | −29.483 | 23.405 | 1.00 | 10.72 |
| ATOM | 12158 | CB | TYR | 1825 | 6.002 | −28.995 | 21.960 | 1.00 | 9.65 |
| ATOM | 12159 | CG | TYR | 1825 | 7.028 | −29.745 | 21.159 | 1.00 | 10.96 |
| ATOM | 12160 | CD1 | TYR | 1825 | 6.640 | −30.696 | 20.226 | 1.00 | 13.24 |
| ATOM | 12161 | CE1 | TYR | 1825 | 7.578 | −31.379 | 19.466 | 1.00 | 13.61 |
| ATOM | 12162 | CD2 | TYR | 1825 | 8.389 | −29.496 | 21.323 | 1.00 | 11.25 |
| ATOM | 12163 | CE2 | TYR | 1825 | 9.341 | −30.180 | 20.569 | 1.00 | 10.63 |
| ATOM | 12164 | CZ | TYR | 1825 | 8.919 | −31.116 | 19.642 | 1.00 | 12.50 |
| ATOM | 12165 | OH | TYR | 1825 | 9.842 | −31.782 | 18.876 | 1.00 | 13.92 |
| ATOM | 12166 | C | TYR | 1825 | 4.614 | −28.854 | 23.994 | 1.00 | 11.54 |
| ATOM | 12167 | O | TYR | 1825 | 4.427 | −27.642 | 23.906 | 1.00 | 12.75 |
| ATOM | 12168 | N | ASP | 1826 | 3.755 | −29.666 | 24.592 | 1.00 | 9.86 |
| ATOM | 12169 | CA | ASP | 1826 | 2.532 | −29.141 | 25.170 | 1.00 | 12.59 |
| ATOM | 12170 | CB | ASP | 1826 | 1.454 | −29.020 | 24.084 | 1.00 | 10.93 |
| ATOM | 12171 | CG | ASP | 1826 | 1.095 | −30.362 | 23.471 | 1.00 | 13.46 |
| ATOM | 12172 | OD1 | ASP | 1826 | 0.357 | −31.125 | 24.116 | 1.00 | 12.97 |
| ATOM | 12173 | OD2 | ASP | 1826 | 1.569 | −30.656 | 22.356 | 1.00 | 14.57 |
| ATOM | 12174 | C | ASP | 1826 | 2.038 | −30.007 | 26.325 | 1.00 | 11.01 |
| ATOM | 12175 | O | ASP | 1826 | 2.516 | −31.120 | 26.549 | 1.00 | 12.69 |
| ATOM | 12176 | N | TYR | 1827 | 1.077 | −29.469 | 27.060 | 1.00 | 11.39 |
| ATOM | 12177 | CA | TYR | 1827 | 0.497 | −30.145 | 28.212 | 1.00 | 11.45 |
| ATOM | 12178 | CB | TYR | 1827 | −0.513 | −29.212 | 28.879 | 1.00 | 12.27 |
| ATOM | 12179 | CG | TYR | 1827 | −1.358 | −29.886 | 29.937 | 1.00 | 12.69 |
| ATOM | 12180 | CD1 | TYR | 1827 | −0.912 | −29.990 | 31.255 | 1.00 | 14.43 |
| ATOM | 12181 | CE1 | TYR | 1827 | −1.696 | −30.593 | 32.234 | 1.00 | 15.64 |
| ATOM | 12182 | CD2 | TYR | 1827 | −2.610 | −30.407 | 29.622 | 1.00 | 14.53 |
| ATOM | 12183 | CE2 | TYR | 1827 | −3.401 | −31.015 | 30.593 | 1.00 | 17.16 |
| ATOM | 12184 | CZ | TYR | 1827 | −2.937 | −31.101 | 31.893 | 1.00 | 16.99 |
| ATOM | 12185 | OH | TYR | 1827 | −3.714 | −31.686 | 32.861 | 1.00 | 16.74 |
| ATOM | 12186 | C | TYR | 1827 | −0.194 | −31.483 | 27.921 | 1.00 | 11.79 |
| ATOM | 12187 | O | TYR | 1827 | −0.010 | −32.474 | 28.632 | 1.00 | 10.69 |
| ATOM | 12188 | N | SER | 1828 | −1.026 | −31.506 | 26.887 | 1.00 | 9.98 |
| ATOM | 12189 | CA | SER | 1828 | −1.779 | −32.713 | 26.553 | 1.00 | 12.49 |
| ATOM | 12190 | CB | SER | 1828 | −2.744 | −32.415 | 25.407 | 1.00 | 11.71 |
| ATOM | 12191 | OG | SER | 1828 | −3.657 | −31.420 | 25.819 | 1.00 | 16.55 |
| ATOM | 12192 | C | SER | 1828 | −0.940 | −33.940 | 26.240 | 1.00 | 11.86 |
| ATOM | 12193 | O | SER | 1828 | −1.210 | −35.027 | 26.763 | 1.00 | 9.82 |
| ATOM | 12194 | N | PHE | 1829 | 0.075 | −33.780 | 25.401 | 1.00 | 11.47 |
| ATOM | 12195 | CA | PHE | 1829 | 0.921 | −34.918 | 25.082 | 1.00 | 11.15 |
| ATOM | 12196 | CB | PHE | 1829 | 1.675 | −34.690 | 23.760 | 1.00 | 10.81 |
| ATOM | 12197 | CG | PHE | 1829 | 0.831 | −34.948 | 22.535 | 1.00 | 10.15 |
| ATOM | 12198 | CD1 | PHE | 1829 | 0.259 | −33.893 | 21.827 | 1.00 | 10.05 |
| ATOM | 12199 | CD2 | PHE | 1829 | 0.566 | −36.256 | 22.120 | 1.00 | 9.31 |
| ATOM | 12200 | CE1 | PHE | 1829 | −0.570 | −34.134 | 20.723 | 1.00 | 9.40 |
| ATOM | 12201 | CE2 | PHE | 1829 | −0.259 | −36.512 | 21.022 | 1.00 | 10.09 |
| ATOM | 12202 | CZ | PHE | 1829 | −0.831 | −35.451 | 20.317 | 1.00 | 9.43 |
| ATOM | 12203 | C | PHE | 1829 | 1.882 | −35.224 | 26.241 | 1.00 | 11.61 |
| ATOM | 12204 | O | PHE | 1829 | 2.161 | −36.391 | 26.531 | 1.00 | 9.86 |
| ATOM | 12205 | N | ALA | 1830 | 2.366 | −34.199 | 26.939 | 1.00 | 9.53 |
| ATOM | 12206 | CA | ALA | 1830 | 3.269 | −34.475 | 28.057 | 1.00 | 11.06 |
| ATOM | 12207 | CB | ALA | 1830 | 3.810 | −33.168 | 28.663 | 1.00 | 13.38 |
| ATOM | 12208 | C | ALA | 1830 | 2.535 | −35.284 | 29.131 | 1.00 | 12.72 |
| ATOM | 12209 | O | ALA | 1830 | 3.116 | −36.172 | 29.759 | 1.00 | 12.34 |
| ATOM | 12210 | N | LYS | 1831 | 1.260 | −34.963 | 29.336 | 1.00 | 10.44 |
| ATOM | 12211 | CA | LYS | 1831 | 0.435 | −35.645 | 30.324 | 1.00 | 12.92 |
| ATOM | 12212 | CB | LYS | 1831 | −0.884 | −34.886 | 30.502 | 1.00 | 15.79 |
| ATOM | 12213 | CG | LYS | 1831 | −1.892 | −35.560 | 31.418 | 1.00 | 19.79 |
| ATOM | 12214 | CD | LYS | 1831 | −1.386 | −35.636 | 32.837 | 1.00 | 26.44 |
| ATOM | 12215 | CE | LYS | 1831 | −2.551 | −35.751 | 33.817 | 1.00 | 30.84 |
| ATOM | 12216 | NZ | LYS | 1831 | −3.543 | −36.796 | 33.410 | 1.00 | 32.55 |
| ATOM | 12217 | C | LYS | 1831 | 0.151 | −37.071 | 29.866 | 1.00 | 10.03 |
| ATOM | 12218 | O | LYS | 1831 | 0.202 | −38.026 | 30.658 | 1.00 | 10.29 |
| ATOM | 12219 | N | LEU | 1832 | −0.158 | −37.211 | 28.582 | 1.00 | 9.47 |
| ATOM | 12220 | CA | LEU | 1832 | −0.449 | −38.529 | 28.017 | 1.00 | 10.13 |
| ATOM | 12221 | CB | LEU | 1832 | −0.811 | −38.397 | 26.532 | 1.00 | 9.08 |
| ATOM | 12222 | CG | LEU | 1832 | −1.337 | −39.651 | 25.819 | 1.00 | 7.77 |
| ATOM | 12223 | CD1 | LEU | 1832 | −2.165 | −39.212 | 24.629 | 1.00 | 10.73 |
| ATOM | 12224 | CD2 | LEU | 1832 | −0.187 | −40.548 | 25.370 | 1.00 | 8.84 |
| ATOM | 12225 | C | LEU | 1832 | 0.764 | −39.434 | 28.195 | 1.00 | 11.17 |
| ATOM | 12226 | O | LEU | 1832 | 0.632 | −40.583 | 28.619 | 1.00 | 10.67 |
| ATOM | 12227 | N | PHE | 1833 | 1.948 | −38.907 | 27.888 | 1.00 | 9.31 |
| ATOM | 12228 | CA | PHE | 1833 | 3.175 | −39.690 | 28.021 | 1.00 | 10.32 |
| ATOM | 12229 | CB | PHE | 1833 | 4.385 | −38.928 | 27.453 | 1.00 | 12.77 |
| ATOM | 12230 | CG | PHE | 1833 | 4.253 | −38.554 | 25.983 | 1.00 | 10.12 |
| ATOM | 12231 | CD1 | PHE | 1833 | 3.502 | −39.331 | 25.105 | 1.00 | 9.09 |
| ATOM | 12232 | CD2 | PHE | 1833 | 4.898 | −37.427 | 25.483 | 1.00 | 11.46 |
| ATOM | 12233 | CE1 | PHE | 1833 | 3.393 | −38.989 | 23.746 | 1.00 | 9.84 |
| ATOM | 12234 | CE2 | PHE | 1833 | 4.798 | −37.074 | 24.125 | 1.00 | 9.94 |
| ATOM | 12235 | CZ | PHE | 1833 | 4.041 | −37.860 | 23.254 | 1.00 | 8.51 |
| ATOM | 12236 | C | PHE | 1833 | 3.438 | −40.043 | 29.486 | 1.00 | 11.08 |
| ATOM | 12237 | O | PHE | 1833 | 3.687 | −41.204 | 29.810 | 1.00 | 11.34 |
| ATOM | 12238 | N | ALA | 1834 | 3.392 | −39.045 | 30.369 | 1.00 | 11.64 |
| ATOM | 12239 | CA | ALA | 1834 | 3.631 | −39.285 | 31.792 | 1.00 | 12.54 |
| ATOM | 12240 | CB | ALA | 1834 | 3.480 | −37.973 | 32.590 | 1.00 | 15.99 |
| ATOM | 12241 | C | ALA | 1834 | 2.692 | −40.349 | 32.367 | 1.00 | 14.96 |
| ATOM | 12242 | O | ALA | 1834 | 3.120 | −41.206 | 33.150 | 1.00 | 13.96 |
| ATOM | 12243 | N | ASP | 1835 | 1.419 | −40.313 | 31.981 | 1.00 | 14.27 |
| ATOM | 12244 | CA | ASP | 1835 | 0.468 | −41.290 | 32.501 | 1.00 | 16.50 |
| ATOM | 12245 | CB | ASP | 1835 | −0.968 | −40.917 | 32.133 | 1.00 | 20.28 |
| ATOM | 12246 | CG | ASP | 1835 | −1.457 | −39.671 | 32.850 | 1.00 | 20.37 |
| ATOM | 12247 | OD1 | ASP | 1835 | −0.859 | −39.275 | 33.875 | 1.00 | 22.29 |
| ATOM | 12248 | OD2 | ASP | 1835 | −2.457 | −39.096 | 32.388 | 1.00 | 25.23 |
| ATOM | 12249 | C | ASP | 1835 | 0.734 | −42.723 | 32.048 | 1.00 | 16.31 |
| ATOM | 12250 | O | ASP | 1835 | 0.176 | −43.671 | 32.612 | 1.00 | 14.66 |
| ATOM | 12251 | N | GLU | 1836 | 1.571 | −42.891 | 31.028 | 1.00 | 15.72 |
| ATOM | 12252 | CA | GLU | 1836 | 1.905 | −44.234 | 30.557 | 1.00 | 14.86 |
| ATOM | 12253 | CB | GLU | 1836 | 1.966 | −44.289 | 29.024 | 1.00 | 13.08 |
| ATOM | 12254 | CG | GLU | 1836 | 0.634 | −44.045 | 28.334 | 1.00 | 13.80 |
| ATOM | 12255 | CD | GLU | 1836 | −0.468 | −44.917 | 28.905 | 1.00 | 18.45 |
| ATOM | 12256 | OE1 | GLU | 1836 | −0.221 | −46.126 | 29.116 | 1.00 | 18.78 |
| ATOM | 12257 | OE2 | GLU | 1836 | −1.578 | −44.395 | 29.144 | 1.00 | 18.26 |
| ATOM | 12258 | C | GLU | 1836 | 3.246 | −44.688 | 31.123 | 1.00 | 15.46 |
| ATOM | 12259 | O | GLU | 1836 | 3.632 | −45.844 | 30.970 | 1.00 | 16.15 |
| ATOM | 12260 | N | GLY | 1837 | 3.962 | −43.770 | 31.762 | 1.00 | 13.82 |
| ATOM | 12261 | CA | GLY | 1837 | 5.252 | −44.112 | 32.336 | 1.00 | 14.92 |
| ATOM | 12262 | C | GLY | 1837 | 6.442 | −43.693 | 31.494 | 1.00 | 14.40 |
| ATOM | 12263 | O | GLY | 1837 | 7.567 | −44.113 | 31.761 | 1.00 | 14.75 |
| ATOM | 12264 | N | LEU | 1838 | 6.197 | −42.879 | 30.466 | 1.00 | 14.07 |
| ATOM | 12265 | CA | LEU | 1838 | 7.263 | −42.384 | 29.588 | 1.00 | 16.57 |
| ATOM | 12266 | CB | LEU | 1838 | 6.703 | −42.123 | 28.188 | 1.00 | 18.28 |
| ATOM | 12267 | CG | LEU | 1838 | 7.637 | −42.371 | 27.005 | 1.00 | 21.26 |
| ATOM | 12268 | CD1 | LEU | 1838 | 6.977 | −41.842 | 25.744 | 1.00 | 18.89 |
| ATOM | 12269 | CD2 | LEU | 1838 | 8.983 | −41.721 | 27.225 | 1.00 | 20.44 |
| ATOM | 12270 | C | LEU | 1838 | 7.642 | −41.073 | 30.267 | 1.00 | 17.17 |
| ATOM | 12271 | O | LEU | 1838 | 7.054 | −40.024 | 29.987 | 1.00 | 16.93 |
| ATOM | 12272 | N | ASN | 1839 | 8.632 | −41.160 | 31.152 | 1.00 | 19.21 |
| ATOM | 12273 | CA | ASN | 1839 | 9.066 | −40.046 | 31.981 | 1.00 | 17.01 |
| ATOM | 12274 | CB | ASN | 1839 | 9.234 | −40.551 | 33.423 | 1.00 | 20.88 |
| ATOM | 12275 | CG | ASN | 1839 | 8.011 | −41.318 | 33.927 | 1.00 | 24.27 |
| ATOM | 12276 | OD1 | ASN | 1839 | 6.867 | −40.885 | 33.742 | 1.00 | 27.75 |
| ATOM | 12277 | ND2 | ASN | 1839 | 8.248 | −42.452 | 34.577 | 1.00 | 26.34 |
| ATOM | 12278 | C | ASN | 1839 | 10.317 | −39.276 | 31.567 | 1.00 | 15.58 |
| ATOM | 12279 | O | ASN | 1839 | 10.956 | −38.627 | 32.397 | 1.00 | 15.41 |
| ATOM | 12280 | N | VAL | 1840 | 10.684 | −39.347 | 30.296 | 1.00 | 14.55 |
| ATOM | 12281 | CA | VAL | 1840 | 11.841 | −38.607 | 29.830 | 1.00 | 13.23 |
| ATOM | 12282 | CB | VAL | 1840 | 13.027 | −39.528 | 29.518 | 1.00 | 13.11 |
| ATOM | 12283 | CG1 | VAL | 1840 | 14.221 | −38.694 | 29.118 | 1.00 | 13.44 |
| ATOM | 12284 | CG2 | VAL | 1840 | 13.348 | −40.392 | 30.748 | 1.00 | 16.08 |
| ATOM | 12285 | C | VAL | 1840 | 11.409 | −37.884 | 28.573 | 1.00 | 12.06 |
| ATOM | 12286 | O | VAL | 1840 | 11.116 | −38.505 | 27.547 | 1.00 | 10.12 |
| ATOM | 12287 | N | MET | 1841 | 11.367 | −36.562 | 28.663 | 1.00 | 10.19 |
| ATOM | 12288 | CA | MET | 1841 | 10.909 | −35.758 | 27.548 | 1.00 | 10.11 |
| ATOM | 12289 | CB | MET | 1841 | 9.522 | −35.193 | 27.873 | 1.00 | 12.54 |
| ATOM | 12290 | CG | MET | 1841 | 8.452 | −36.273 | 27.916 | 1.00 | 15.69 |
| ATOM | 12291 | SD | MET | 1841 | 6.923 | −35.649 | 28.511 | 1.00 | 15.98 |
| ATOM | 12292 | CE | MET | 1841 | 6.633 | −36.759 | 29.903 | 1.00 | 13.37 |
| ATOM | 12293 | C | MET | 1841 | 11.864 | −34.647 | 27.150 | 1.00 | 10.13 |
| ATOM | 12294 | O | MET | 1841 | 12.468 | −33.969 | 27.992 | 1.00 | 9.95 |
| ATOM | 12295 | N | LEU | 1842 | 11.959 | −34.456 | 25.839 | 1.00 | 10.43 |
| ATOM | 12296 | CA | LEU | 1842 | 12.838 | −33.454 | 25.264 | 1.00 | 11.43 |
| ATOM | 12297 | CB | LEU | 1842 | 13.804 | −34.123 | 24.283 | 1.00 | 14.79 |
| ATOM | 12298 | CG | LEU | 1842 | 14.979 | −33.359 | 23.651 | 1.00 | 20.32 |
| ATOM | 12299 | CD1 | LEU | 1842 | 14.650 | −33.033 | 22.220 | 1.00 | 23.15 |
| ATOM | 12300 | CD2 | LEU | 1842 | 15.339 | −32.102 | 24.428 | 1.00 | 19.90 |
| ATOM | 12301 | C | LEU | 1842 | 12.060 | −32.349 | 24.566 | 1.00 | 11.63 |
| ATOM | 12302 | O | LEU | 1842 | 11.246 | −32.610 | 23.687 | 1.00 | 9.65 |
| ATOM | 12303 | N | VAL | 1843 | 12.299 | −31.115 | 25.001 | 1.00 | 11.18 |
| ATOM | 12304 | CA | VAL | 1843 | 11.670 | −29.947 | 24.395 | 1.00 | 12.10 |
| ATOM | 12305 | CB | VAL | 1843 | 11.215 | −28.917 | 25.454 | 1.00 | 12.21 |
| ATOM | 12306 | CG1 | VAL | 1843 | 10.567 | −27.708 | 24.763 | 1.00 | 10.58 |
| ATOM | 12307 | CG2 | VAL | 1843 | 10.212 | −29.564 | 26.405 | 1.00 | 15.96 |
| ATOM | 12308 | C | VAL | 1843 | 12.800 | −29.383 | 23.548 | 1.00 | 13.23 |
| ATOM | 12309 | O | VAL | 1843 | 13.601 | −28.580 | 24.019 | 1.00 | 13.64 |
| ATOM | 12310 | N | GLY | 1844 | 12.877 | −29.837 | 22.302 | 1.00 | 14.75 |
| ATOM | 12311 | CA | GLY | 1844 | 13.950 | −29.406 | 21.424 | 1.00 | 15.77 |
| ATOM | 12312 | C | GLY | 1844 | 13.563 | −28.393 | 20.375 | 1.00 | 13.72 |
| ATOM | 12313 | O | GLY | 1844 | 12.376 | −28.167 | 20.127 | 1.00 | 11.89 |
| ATOM | 12314 | N | ASP | 1845 | 14.559 | −27.781 | 19.742 | 1.00 | 14.76 |
| ATOM | 12315 | CA | ASP | 1845 | 14.246 | −26.776 | 18.732 | 1.00 | 14.86 |
| ATOM | 12316 | CB | ASP | 1845 | 15.485 | −25.952 | 18.342 | 1.00 | 16.86 |
| ATOM | 12317 | CG | ASP | 1845 | 16.649 | −26.794 | 17.857 | 1.00 | 17.94 |
| ATOM | 12318 | OD1 | ASP | 1845 | 16.470 | −27.985 | 17.559 | 1.00 | 16.65 |
| ATOM | 12319 | OD2 | ASP | 1845 | 17.757 | −26.227 | 17.768 | 1.00 | 19.70 |
| ATOM | 12320 | C | ASP | 1845 | 13.567 | −27.368 | 17.506 | 1.00 | 14.35 |
| ATOM | 12321 | O | ASP | 1845 | 13.185 | −26.641 | 16.588 | 1.00 | 14.70 |
| ATOM | 12322 | N | SER | 1846 | 13.401 | −28.689 | 17.493 | 1.00 | 14.18 |
| ATOM | 12323 | CA | SER | 1846 | 12.699 | −29.334 | 16.385 | 1.00 | 12.45 |
| ATOM | 12324 | CB | SER | 1846 | 12.615 | −30.848 | 16.607 | 1.00 | 14.08 |
| ATOM | 12325 | OG | SER | 1846 | 12.079 | −31.168 | 17.888 | 1.00 | 16.33 |
| ATOM | 12326 | C | SER | 1846 | 11.291 | −28.735 | 16.337 | 1.00 | 12.85 |
| ATOM | 12327 | O | SER | 1846 | 10.600 | −28.821 | 15.316 | 1.00 | 13.17 |
| ATOM | 12328 | N | LEU | 1847 | 10.858 | −28.146 | 17.451 | 1.00 | 10.11 |
| ATOM | 12329 | CA | LEU | 1847 | 9.532 | −27.517 | 17.503 | 1.00 | 9.80 |
| ATOM | 12330 | CB | LEU | 1847 | 9.242 | −26.969 | 18.911 | 1.00 | 8.90 |
| ATOM | 12331 | CG | LEU | 1847 | 10.043 | −25.770 | 19.432 | 1.00 | 9.46 |
| ATOM | 12332 | CD1 | LEU | 1847 | 9.440 | −24.474 | 18.904 | 1.00 | 6.94 |
| ATOM | 12333 | CD2 | LEU | 1847 | 10.039 | −25.763 | 20.968 | 1.00 | 10.36 |
| ATOM | 12334 | C | LEU | 1847 | 9.436 | −26.391 | 16.466 | 1.00 | 8.37 |
| ATOM | 12335 | O | LEU | 1847 | 8.341 | −26.001 | 16.051 | 1.00 | 9.72 |
| ATOM | 12336 | N | GLY | 1848 | 10.585 | −25.861 | 16.060 | 1.00 | 7.80 |
| ATOM | 12337 | CA | GLY | 1848 | 10.594 | −24.804 | 15.061 | 1.00 | 9.90 |
| ATOM | 12338 | C | GLY | 1848 | 9.921 | −25.270 | 13.783 | 1.00 | 12.28 |
| ATOM | 12339 | O | GLY | 1848 | 9.277 | −24.495 | 13.077 | 1.00 | 11.95 |
| ATOM | 12340 | N | MET | 1849 | 10.048 | −26.556 | 13.485 | 1.00 | 12.66 |
| ATOM | 12341 | CA | MET | 1849 | 9.446 | −27.088 | 12.273 | 1.00 | 14.41 |
| ATOM | 12342 | CB | MET | 1849 | 10.406 | −28.092 | 11.641 | 1.00 | 18.14 |
| ATOM | 12343 | CG | MET | 1849 | 11.766 | −27.472 | 11.327 | 1.00 | 19.85 |
| ATOM | 12344 | SD | MET | 1849 | 12.956 | −28.634 | 10.655 | 1.00 | 24.17 |
| ATOM | 12345 | CE | MET | 1849 | 12.389 | −28.777 | 8.968 | 1.00 | 24.76 |
| ATOM | 12346 | C | MET | 1849 | 8.093 | −27.724 | 12.538 | 1.00 | 12.05 |
| ATOM | 12347 | O | MET | 1849 | 7.097 | −27.396 | 11.893 | 1.00 | 13.07 |
| ATOM | 12348 | N | THR | 1850 | 8.052 | −28.613 | 13.518 | 1.00 | 13.89 |
| ATOM | 12349 | CA | THR | 1850 | 6.826 | −29.321 | 13.852 | 1.00 | 13.07 |
| ATOM | 12350 | CB | THR | 1850 | 7.165 | −30.523 | 14.756 | 1.00 | 16.49 |
| ATOM | 12351 | OG1 | THR | 1850 | 6.003 | −31.332 | 14.935 | 1.00 | 23.16 |
| ATOM | 12352 | CG2 | THR | 1850 | 7.657 | −30.044 | 16.106 | 1.00 | 13.78 |
| ATOM | 12353 | C | THR | 1850 | 5.722 | −28.477 | 14.502 | 1.00 | 13.45 |
| ATOM | 12354 | O | THR | 1850 | 4.529 | −28.704 | 14.272 | 1.00 | 14.06 |
| ATOM | 12355 | N | VAL | 1851 | 6.101 | −27.505 | 15.318 | 1.00 | 9.38 |
| ATOM | 12356 | CA | VAL | 1851 | 5.095 | −26.662 | 15.963 | 1.00 | 10.18 |
| ATOM | 12357 | CB | VAL | 1851 | 5.447 | −26.405 | 17.456 | 1.00 | 9.38 |
| ATOM | 12358 | CG1 | VAL | 1851 | 4.436 | −25.440 | 18.073 | 1.00 | 9.31 |
| ATOM | 12359 | CG2 | VAL | 1851 | 5.440 | −27.719 | 18.219 | 1.00 | 9.77 |
| ATOM | 12360 | C | VAL | 1851 | 4.914 | −25.316 | 15.262 | 1.00 | 10.37 |
| ATOM | 12361 | O | VAL | 1851 | 3.795 | −24.940 | 14.935 | 1.00 | 10.77 |
| ATOM | 12362 | N | GLN | 1852 | 6.017 | −24.604 | 15.025 | 1.00 | 9.98 |
| ATOM | 12363 | CA | GLN | 1852 | 5.959 | −23.283 | 14.405 | 1.00 | 10.14 |
| ATOM | 12364 | CB | GLN | 1852 | 7.198 | −22.486 | 14.801 | 1.00 | 10.92 |
| ATOM | 12365 | CG | GLN | 1852 | 7.379 | −22.402 | 16.313 | 1.00 | 11.64 |
| ATOM | 12366 | CD | GLN | 1852 | 8.590 | −21.591 | 16.702 | 1.00 | 11.56 |
| ATOM | 12367 | OE1 | GLN | 1852 | 9.567 | −21.530 | 15.963 | 1.00 | 11.26 |
| ATOM | 12368 | NE2 | GLN | 1852 | 8.545 | −20.990 | 17.882 | 1.00 | 8.34 |
| ATOM | 12369 | C | GLN | 1852 | 5.804 | −23.277 | 12.891 | 1.00 | 11.52 |
| ATOM | 12370 | O | GLN | 1852 | 5.301 | −22.314 | 12.315 | 1.00 | 10.45 |
| ATOM | 12371 | N | GLY | 1853 | 6.257 | −24.340 | 12.239 | 1.00 | 9.89 |
| ATOM | 12372 | CA | GLY | 1853 | 6.119 | −24.422 | 10.793 | 1.00 | 10.59 |
| ATOM | 12373 | C | GLY | 1853 | 7.217 | −23.823 | 9.941 | 1.00 | 11.71 |
| ATOM | 12374 | O | GLY | 1853 | 6.991 | −23.534 | 8.766 | 1.00 | 12.91 |
| ATOM | 12375 | N | HIS | 1854 | 8.395 | −23.618 | 10.522 | 1.00 | 11.61 |
| ATOM | 12376 | CA | HIS | 1854 | 9.527 | −23.076 | 9.768 | 1.00 | 14.51 |
| ATOM | 12377 | CB | HIS | 1854 | 10.534 | −22.413 | 10.704 | 1.00 | 12.24 |
| ATOM | 12378 | CG | HIS | 1854 | 9.987 | −21.230 | 11.432 | 1.00 | 13.00 |
| ATOM | 12379 | CD2 | HIS | 1854 | 9.726 | −21.033 | 12.747 | 1.00 | 11.78 |
| ATOM | 12380 | ND1 | HIS | 1854 | 9.636 | −20.060 | 10.792 | 1.00 | 11.06 |
| ATOM | 12381 | CE1 | HIS | 1854 | 9.186 | −19.193 | 11.682 | 1.00 | 13.39 |
| ATOM | 12382 | NE2 | HIS | 1854 | 9.231 | −19.758 | 12.875 | 1.00 | 12.54 |
| ATOM | 12383 | C | HIS | 1854 | 10.210 | −24.225 | 9.037 | 1.00 | 15.33 |
| ATOM | 12384 | O | HIS | 1854 | 10.034 | −25.389 | 9.395 | 1.00 | 14.84 |
| ATOM | 12385 | N | ASP | 1855 | 11.005 | −23.888 | 8.028 | 1.00 | 18.03 |
| ATOM | 12386 | CA | ASP | 1855 | 11.718 | −24.871 | 7.224 | 1.00 | 20.65 |
| ATOM | 12387 | CB | ASP | 1855 | 12.081 | −24.247 | 5.871 | 1.00 | 26.04 |
| ATOM | 12388 | CG | ASP | 1855 | 13.150 | −23.172 | 5.999 | 1.00 | 27.76 |
| ATOM | 12389 | OD1 | ASP | 1855 | 14.313 | −23.526 | 6.253 | 1.00 | 35.96 |
| ATOM | 12390 | OD2 | ASP | 1855 | 12.827 | −21.977 | 5.864 | 1.00 | 34.52 |
| ATOM | 12391 | C | ASP | 1855 | 12.993 | −25.347 | 7.928 | 1.00 | 19.44 |
| ATOM | 12392 | O | ASP | 1855 | 13.645 | −26.288 | 7.477 | 1.00 | 17.58 |
| ATOM | 12393 | N | SER | 1856 | 13.352 | −24.685 | 9.026 | 1.00 | 17.83 |
| ATOM | 12394 | CA | SER | 1856 | 14.540 | −25.050 | 9.794 | 1.00 | 15.27 |
| ATOM | 12395 | CB | SER | 1856 | 15.762 | −24.257 | 9.323 | 1.00 | 16.75 |
| ATOM | 12396 | OG | SER | 1856 | 15.761 | −22.953 | 9.884 | 1.00 | 13.49 |
| ATOM | 12397 | C | SER | 1856 | 14.289 | −24.735 | 11.260 | 1.00 | 14.46 |
| ATOM | 12398 | O | SER | 1856 | 13.259 | −24.163 | 11.613 | 1.00 | 13.21 |
| ATOM | 12399 | N | THR | 1857 | 15.244 | −25.085 | 12.111 | 1.00 | 15.25 |
| ATOM | 12400 | CA | THR | 1857 | 15.094 | −24.823 | 13.536 | 1.00 | 14.62 |
| ATOM | 12401 | CB | THR | 1857 | 15.712 | −25.947 | 14.392 | 1.00 | 15.79 |
| ATOM | 12402 | OG1 | THR | 1857 | 17.125 | −25.993 | 14.169 | 1.00 | 14.91 |
| ATOM | 12403 | CG2 | THR | 1857 | 15.106 | −27.286 | 14.042 | 1.00 | 15.97 |
| ATOM | 12404 | C | THR | 1857 | 15.759 | −23.516 | 13.964 | 1.00 | 12.81 |
| ATOM | 12405 | O | THR | 1857 | 15.666 | −23.145 | 15.128 | 1.00 | 13.80 |
| ATOM | 12406 | N | LEU | 1858 | 16.425 | −22.815 | 13.047 | 1.00 | 12.35 |
| ATOM | 12407 | CA | LEU | 1858 | 17.119 | −21.582 | 13.434 | 1.00 | 12.54 |
| ATOM | 12408 | CB | LEU | 1858 | 17.920 | −21.013 | 12.252 | 1.00 | 12.41 |
| ATOM | 12409 | CG | LEU | 1858 | 19.243 | −21.743 | 11.959 | 1.00 | 18.92 |
| ATOM | 12410 | CD1 | LEU | 1858 | 18.942 | −23.123 | 11.376 | 1.00 | 18.27 |
| ATOM | 12411 | CD2 | LEU | 1858 | 20.079 | −20.929 | 10.975 | 1.00 | 16.42 |
| ATOM | 12412 | C | LEU | 1858 | 16.269 | −20.465 | 14.049 | 1.00 | 12.00 |
| ATOM | 12413 | O | LEU | 1858 | 16.729 | −19.749 | 14.943 | 1.00 | 12.79 |
| ATOM | 12414 | N | PRO | 1859 | 15.021 | −20.296 | 13.587 | 1.00 | 11.97 |
| ATOM | 12415 | CD | PRO | 1859 | 14.411 | −20.851 | 12.366 | 1.00 | 15.36 |
| ATOM | 12416 | CA | PRO | 1859 | 14.190 | −19.228 | 14.159 | 1.00 | 12.87 |
| ATOM | 12417 | CB | PRO | 1859 | 12.981 | −19.178 | 13.222 | 1.00 | 14.66 |
| ATOM | 12418 | CG | PRO | 1859 | 12.969 | −20.528 | 12.567 | 1.00 | 20.26 |
| ATOM | 12419 | C | PRO | 1859 | 13.789 | −19.393 | 15.623 | 1.00 | 12.26 |
| ATOM | 12420 | O | PRO | 1859 | 13.431 | −18.418 | 16.287 | 1.00 | 11.90 |
| ATOM | 12421 | N | VAL | 1860 | 13.853 | −20.620 | 16.125 | 1.00 | 9.89 |
| ATOM | 12422 | CA | VAL | 1860 | 13.487 | −20.900 | 17.514 | 1.00 | 9.80 |
| ATOM | 12423 | CB | VAL | 1860 | 13.650 | −22.397 | 17.824 | 1.00 | 9.99 |
| ATOM | 12424 | CG1 | VAL | 1860 | 13.225 | −22.674 | 19.258 | 1.00 | 10.58 |
| ATOM | 12425 | CG2 | VAL | 1860 | 12.816 | −23.222 | 16.855 | 1.00 | 10.13 |
| ATOM | 12426 | C | VAL | 1860 | 14.343 | −20.116 | 18.507 | 1.00 | 9.52 |
| ATOM | 12427 | O | VAL | 1860 | 15.570 | −20.165 | 18.449 | 1.00 | 9.57 |
| ATOM | 12428 | N | THR | 1861 | 13.691 | −19.419 | 19.435 | 1.00 | 9.74 |
| ATOM | 12429 | CA | THR | 1861 | 14.412 | −18.633 | 20.432 | 1.00 | 12.01 |
| ATOM | 12430 | CB | THR | 1861 | 13.845 | −17.199 | 20.509 | 1.00 | 15.97 |
| ATOM | 12431 | OG1 | THR | 1861 | 13.816 | −16.623 | 19.192 | 1.00 | 20.58 |
| ATOM | 12432 | CG2 | THR | 1861 | 14.722 | −16.325 | 21.387 | 1.00 | 23.39 |
| ATOM | 12433 | C | THR | 1861 | 14.367 | −19.270 | 21.822 | 1.00 | 11.32 |
| ATOM | 12434 | O | THR | 1861 | 13.603 | −20.199 | 22.072 | 1.00 | 9.35 |
| ATOM | 12435 | N | VAL | 1862 | 15.214 | −18.773 | 22.717 | 1.00 | 11.39 |
| ATOM | 12436 | CA | VAL | 1862 | 15.251 | −19.275 | 24.079 | 1.00 | 11.60 |
| ATOM | 12437 | CB | VAL | 1862 | 16.309 | −18.524 | 24.920 | 1.00 | 11.99 |
| ATOM | 12438 | CG1 | VAL | 1862 | 16.238 | −18.982 | 26.367 | 1.00 | 13.48 |
| ATOM | 12439 | CG2 | VAL | 1862 | 17.715 | −18.775 | 24.349 | 1.00 | 13.06 |
| ATOM | 12440 | C | VAL | 1862 | 13.864 | −19.080 | 24.695 | 1.00 | 11.31 |
| ATOM | 12441 | O | VAL | 1862 | 13.366 | −19.955 | 25.398 | 1.00 | 11.62 |
| ATOM | 12442 | N | ALA | 1863 | 13.227 | −17.941 | 24.421 | 1.00 | 10.93 |
| ATOM | 12443 | CA | ALA | 1863 | 11.893 | −17.697 | 24.966 | 1.00 | 10.99 |
| ATOM | 12444 | CB | ALA | 1863 | 11.380 | −16.336 | 24.535 | 1.00 | 13.54 |
| ATOM | 12445 | C | ALA | 1863 | 10.925 | −18.773 | 24.499 | 1.00 | 10.75 |
| ATOM | 12446 | O | ALA | 1863 | 10.087 | −19.240 | 25.278 | 1.00 | 8.92 |
| ATOM | 12447 | N | ASP | 1864 | 11.013 | −19.141 | 23.221 | 1.00 | 8.92 |
| ATOM | 12448 | CA | ASP | 1864 | 10.135 | −20.191 | 22.669 | 1.00 | 9.84 |
| ATOM | 12449 | CB | ASP | 1864 | 10.452 | −20.488 | 21.191 | 1.00 | 9.14 |
| ATOM | 12450 | CG | ASP | 1864 | 10.169 | −19.322 | 20.263 | 1.00 | 9.13 |
| ATOM | 12451 | OD1 | ASP | 1864 | 9.190 | −18.581 | 20.490 | 1.00 | 11.77 |
| ATOM | 12452 | OD2 | ASP | 1864 | 10.918 | −19.167 | 19.272 | 1.00 | 10.86 |
| ATOM | 12453 | C | ASP | 1864 | 10.328 | −21.487 | 23.456 | 1.00 | 9.58 |
| ATOM | 12454 | O | ASP | 1864 | 9.358 | −22.134 | 23.861 | 1.00 | 8.41 |
| ATOM | 12455 | N | ILE | 1865 | 11.583 | −21.876 | 23.660 | 1.00 | 8.67 |
| ATOM | 12456 | CA | ILE | 1865 | 11.879 | −23.111 | 24.398 | 1.00 | 9.31 |
| ATOM | 12457 | CB | ILE | 1865 | 13.409 | −23.355 | 24.520 | 1.00 | 9.33 |
| ATOM | 12458 | CG2 | ILE | 1865 | 13.668 | −24.518 | 25.470 | 1.00 | 11.52 |
| ATOM | 12459 | CG1 | ILE | 1865 | 14.035 | −23.606 | 23.134 | 1.00 | 9.79 |
| ATOM | 12460 | CD1 | ILE | 1865 | 13.509 | −24.855 | 22.397 | 1.00 | 8.07 |
| ATOM | 12461 | C | ILE | 1865 | 11.285 | −23.036 | 25.808 | 1.00 | 9.25 |
| ATOM | 12462 | O | ILE | 1865 | 10.661 | −23.991 | 26.283 | 1.00 | 9.30 |
| ATOM | 12463 | N | ALA | 1866 | 11.492 | −21.906 | 26.483 | 1.00 | 7.42 |
| ATOM | 12464 | CA | ALA | 1866 | 10.969 | −21.716 | 27.843 | 1.00 | 5.92 |
| ATOM | 12465 | CB | ALA | 1866 | 11.352 | −20.335 | 28.373 | 1.00 | 6.24 |
| ATOM | 12466 | C | ALA | 1866 | 9.452 | −21.879 | 27.895 | 1.00 | 8.68 |
| ATOM | 12467 | O | ALA | 1866 | 8.904 | −22.480 | 28.826 | 1.00 | 6.71 |
| ATOM | 12468 | N | TYR | 1867 | 8.768 | −21.319 | 26.903 | 1.00 | 7.88 |
| ATOM | 12469 | CA | TYR | 1867 | 7.307 | −21.421 | 26.817 | 1.00 | 9.45 |
| ATOM | 12470 | CB | TYR | 1867 | 6.799 | −20.700 | 25.550 | 1.00 | 8.36 |
| ATOM | 12471 | CG | TYR | 1867 | 5.304 | −20.839 | 25.314 | 1.00 | 8.40 |
| ATOM | 12472 | CD1 | TYR | 1867 | 4.384 | −20.257 | 26.184 | 1.00 | 11.09 |
| ATOM | 12473 | CE1 | TYR | 1867 | 3.010 | −20.316 | 25.930 | 1.00 | 11.97 |
| ATOM | 12474 | CD2 | TYR | 1867 | 4.812 | −21.496 | 24.187 | 1.00 | 9.92 |
| ATOM | 12475 | CE2 | TYR | 1867 | 3.440 | −21.561 | 23.925 | 1.00 | 9.81 |
| ATOM | 12476 | CZ | TYR | 1867 | 2.550 | −20.968 | 24.791 | 1.00 | 9.83 |
| ATOM | 12477 | OH | TYR | 1867 | 1.204 | −20.972 | 24.502 | 1.00 | 13.41 |
| ATOM | 12478 | C | TYR | 1867 | 6.844 | −22.874 | 26.773 | 1.00 | 9.90 |
| ATOM | 12479 | O | TYR | 1867 | 5.984 | −23.308 | 27.556 | 1.00 | 8.93 |
| ATOM | 12480 | N | HIS | 1868 | 7.413 | −23.629 | 25.843 | 1.00 | 7.87 |
| ATOM | 12481 | CA | HIS | 1868 | 7.025 | −25.026 | 25.691 | 1.00 | 10.27 |
| ATOM | 12482 | CB | HIS | 1868 | 7.557 | −25.543 | 24.349 | 1.00 | 9.57 |
| ATOM | 12483 | CG | HIS | 1868 | 6.843 | −24.939 | 23.174 | 1.00 | 9.11 |
| ATOM | 12484 | CD2 | HIS | 1868 | 7.194 | −23.938 | 22.330 | 1.00 | 9.03 |
| ATOM | 12485 | ND1 | HIS | 1868 | 5.543 | −25.267 | 22.852 | 1.00 | 8.93 |
| ATOM | 12486 | CE1 | HIS | 1868 | 5.120 | −24.490 | 21.870 | 1.00 | 8.96 |
| ATOM | 12487 | NE2 | HIS | 1868 | 6.102 | −23.674 | 21.534 | 1.00 | 8.75 |
| ATOM | 12488 | C | HIS | 1868 | 7.483 | −25.860 | 26.874 | 1.00 | 10.42 |
| ATOM | 12489 | O | HIS | 1868 | 6.800 | −26.804 | 27.266 | 1.00 | 9.01 |
| ATOM | 12490 | N | THR | 1869 | 8.611 | −25.481 | 27.469 | 1.00 | 10.19 |
| ATOM | 12491 | CA | THR | 1869 | 9.133 | −26.197 | 28.635 | 1.00 | 10.01 |
| ATOM | 12492 | CB | THR | 1869 | 10.520 | −25.639 | 29.032 | 1.00 | 11.08 |
| ATOM | 12493 | OG1 | THR | 1869 | 11.474 | −25.995 | 28.022 | 1.00 | 11.79 |
| ATOM | 12494 | CG2 | THR | 1869 | 10.984 | −26.205 | 30.378 | 1.00 | 10.61 |
| ATOM | 12495 | C | THR | 1869 | 8.156 | −26.077 | 29.809 | 1.00 | 10.50 |
| ATOM | 12496 | O | THR | 1869 | 7.896 | −27.056 | 30.501 | 1.00 | 9.02 |
| ATOM | 12497 | N | ALA | 1870 | 7.607 | −24.884 | 30.036 | 1.00 | 9.46 |
| ATOM | 12498 | CA | ALA | 1870 | 6.673 | −24.719 | 31.146 | 1.00 | 10.75 |
| ATOM | 12499 | CB | ALA | 1870 | 6.301 | −23.242 | 31.331 | 1.00 | 11.04 |
| ATOM | 12500 | C | ALA | 1870 | 5.416 | −25.553 | 30.909 | 1.00 | 10.58 |
| ATOM | 12501 | O | ALA | 1870 | 4.886 | −26.180 | 31.831 | 1.00 | 11.31 |
| ATOM | 12502 | N | ALA | 1871 | 4.939 | −25.568 | 29.672 | 1.00 | 9.74 |
| ATOM | 12503 | CA | ALA | 1871 | 3.738 | −26.332 | 29.347 | 1.00 | 10.34 |
| ATOM | 12504 | CB | ALA | 1871 | 3.323 | −26.056 | 27.907 | 1.00 | 9.64 |
| ATOM | 12505 | C | ALA | 1871 | 3.965 | −27.828 | 29.559 | 1.00 | 10.87 |
| ATOM | 12506 | O | ALA | 1871 | 3.104 | −28.514 | 30.116 | 1.00 | 11.14 |
| ATOM | 12507 | N | VAL | 1872 | 5.119 | −28.333 | 29.126 | 1.00 | 8.50 |
| ATOM | 12508 | CA | VAL | 1872 | 5.418 | −29.754 | 29.300 | 1.00 | 9.80 |
| ATOM | 12509 | CB | VAL | 1872 | 6.724 | −30.161 | 28.569 | 1.00 | 8.60 |
| ATOM | 12510 | CG1 | VAL | 1872 | 7.169 | −31.563 | 29.019 | 1.00 | 5.70 |
| ATOM | 12511 | CG2 | VAL | 1872 | 6.483 | −30.174 | 27.061 | 1.00 | 8.96 |
| ATOM | 12512 | C | VAL | 1872 | 5.518 | −30.078 | 30.788 | 1.00 | 10.76 |
| ATOM | 12513 | O | VAL | 1872 | 4.979 | −31.085 | 31.245 | 1.00 | 11.38 |
| ATOM | 12514 | N | ARG | 1873 | 6.192 | −29.221 | 31.549 | 1.00 | 10.87 |
| ATOM | 12515 | CA | ARG | 1873 | 6.311 | −29.450 | 32.980 | 1.00 | 9.93 |
| ATOM | 12516 | CB | ARG | 1873 | 7.153 | −28.348 | 33.647 | 1.00 | 7.52 |
| ATOM | 12517 | CG | ARG | 1873 | 7.288 | −28.505 | 35.177 | 1.00 | 10.26 |
| ATOM | 12518 | CD | ARG | 1873 | 7.857 | −29.878 | 35.567 | 1.00 | 12.74 |
| ATOM | 12519 | NE | ARG | 1873 | 9.289 | −30.011 | 35.288 | 1.00 | 15.69 |
| ATOM | 12520 | CZ | ARG | 1873 | 9.939 | −31.172 | 35.250 | 1.00 | 12.78 |
| ATOM | 12521 | NH1 | ARG | 1873 | 9.290 | −32.304 | 35.461 | 1.00 | 13.43 |
| ATOM | 12522 | NH2 | ARG | 1873 | 11.242 | −31.205 | 35.025 | 1.00 | 12.86 |
| ATOM | 12523 | C | ARG | 1873 | 4.924 | −29.522 | 33.645 | 1.00 | 10.69 |
| ATOM | 12524 | O | ARG | 1873 | 4.726 | −30.310 | 34.562 | 1.00 | 12.04 |
| ATOM | 12525 | N | ARG | 1874 | 3.968 | −28.713 | 33.190 | 1.00 | 9.73 |
| ATOM | 12526 | CA | ARG | 1874 | 2.631 | −28.750 | 33.774 | 1.00 | 10.96 |
| ATOM | 12527 | CB | ARG | 1874 | 1.736 | −27.640 | 33.206 | 1.00 | 12.21 |
| ATOM | 12528 | CG | ARG | 1874 | 2.285 | −26.245 | 33.444 | 1.00 | 15.36 |
| ATOM | 12529 | CD | ARG | 1874 | 1.230 | −25.139 | 33.364 | 1.00 | 17.99 |
| ATOM | 12530 | NE | ARG | 1874 | 1.868 | −23.843 | 33.592 | 1.00 | 15.73 |
| ATOM | 12531 | CZ | ARG | 1874 | 2.419 | −23.101 | 32.637 | 1.00 | 19.11 |
| ATOM | 12532 | NH1 | ARG | 1874 | 2.397 | −23.517 | 31.373 | 1.00 | 14.81 |
| ATOM | 12533 | NH2 | ARG | 1874 | 3.027 | −21.961 | 32.955 | 1.00 | 17.47 |
| ATOM | 12534 | C | ARG | 1874 | 1.970 | −30.110 | 33.546 | 1.00 | 11.41 |
| ATOM | 12535 | O | ARG | 1874 | 1.273 | −30.630 | 34.424 | 1.00 | 14.88 |
| ATOM | 12536 | N | GLY | 1875 | 2.200 | −30.696 | 32.380 | 1.00 | 11.22 |
| ATOM | 12537 | CA | GLY | 1875 | 1.606 | −31.990 | 32.077 | 1.00 | 10.19 |
| ATOM | 12538 | C | GLY | 1875 | 2.323 | −33.162 | 32.720 | 1.00 | 10.02 |
| ATOM | 12539 | O | GLY | 1875 | 1.708 | −34.182 | 33.021 | 1.00 | 9.60 |
| ATOM | 12540 | N | ALA | 1876 | 3.627 | −33.012 | 32.935 | 1.00 | 11.21 |
| ATOM | 12541 | CA | ALA | 1876 | 4.455 | −34.071 | 33.527 | 1.00 | 11.50 |
| ATOM | 12542 | CB | ALA | 1876 | 5.309 | −34.718 | 32.430 | 1.00 | 14.27 |
| ATOM | 12543 | C | ALA | 1876 | 5.350 | −33.513 | 34.633 | 1.00 | 12.82 |
| ATOM | 12544 | O | ALA | 1876 | 6.560 | −33.374 | 34.453 | 1.00 | 11.92 |
| ATOM | 12545 | N | PRO | 1877 | 4.771 | −33.210 | 35.806 | 1.00 | 14.04 |
| ATOM | 12546 | CD | PRO | 1877 | 3.375 | −33.476 | 36.216 | 1.00 | 14.97 |
| ATOM | 12547 | CA | PRO | 1877 | 5.554 | −32.657 | 36.920 | 1.00 | 15.62 |
| ATOM | 12548 | CB | PRO | 1877 | 4.491 | −32.375 | 37.984 | 1.00 | 15.70 |
| ATOM | 12549 | CG | PRO | 1877 | 3.469 | −33.421 | 37.736 | 1.00 | 18.20 |
| ATOM | 12550 | C | PRO | 1877 | 6.699 | −33.510 | 37.462 | 1.00 | 16.14 |
| ATOM | 12551 | O | PRO | 1877 | 7.589 | −32.992 | 38.132 | 1.00 | 18.76 |
| ATOM | 12552 | N | ASN | 1878 | 6.686 | −34.801 | 37.146 | 1.00 | 15.89 |
| ATOM | 12553 | CA | ASN | 1878 | 7.709 | −35.718 | 37.638 | 1.00 | 17.91 |
| ATOM | 12554 | CB | ASN | 1878 | 7.038 | −36.936 | 38.288 | 1.00 | 22.40 |
| ATOM | 12555 | CG | ASN | 1878 | 6.081 | −36.552 | 39.400 | 1.00 | 25.24 |
| ATOM | 12556 | OD1 | ASN | 1878 | 6.463 | −35.889 | 40.359 | 1.00 | 29.66 |
| ATOM | 12557 | ND2 | ASN | 1878 | 4.824 | −36.974 | 39.275 | 1.00 | 28.39 |
| ATOM | 12558 | C | ASN | 1878 | 8.710 | −36.209 | 36.595 | 1.00 | 17.02 |
| ATOM | 12559 | O | ASN | 1878 | 9.604 | −36.986 | 36.919 | 1.00 | 16.69 |
| ATOM | 12560 | N | CYS | 1879 | 8.578 | −35.763 | 35.349 | 1.00 | 15.26 |
| ATOM | 12561 | CA | CYS | 1879 | 9.487 | −36.238 | 34.309 | 1.00 | 13.68 |
| ATOM | 12562 | CB | CYS | 1879 | 8.890 | −35.991 | 32.917 | 1.00 | 14.20 |
| ATOM | 12563 | SG | CYS | 1879 | 9.037 | −34.260 | 32.286 | 1.00 | 16.39 |
| ATOM | 12564 | C | CYS | 1879 | 10.865 | −35.596 | 34.365 | 1.00 | 13.72 |
| ATOM | 12565 | O | CYS | 1879 | 11.085 | −34.604 | 35.062 | 1.00 | 13.75 |
| ATOM | 12566 | N | LEU | 1880 | 11.802 | −36.213 | 33.658 | 1.00 | 13.56 |
| ATOM | 12567 | CA | LEU | 1880 | 13.130 | −35.660 | 33.540 | 1.00 | 13.22 |
| ATOM | 12568 | CB | LEU | 1880 | 14.185 | −36.740 | 33.317 | 1.00 | 12.70 |
| ATOM | 12569 | CG | LEU | 1880 | 15.583 | −36.162 | 33.044 | 1.00 | 12.13 |
| ATOM | 12570 | CD1 | LEU | 1880 | 16.056 | −35.315 | 34.242 | 1.00 | 14.50 |
| ATOM | 12571 | CD2 | LEU | 1880 | 16.561 | −37.299 | 32.782 | 1.00 | 15.41 |
| ATOM | 12572 | C | LEU | 1880 | 12.925 | −34.860 | 32.259 | 1.00 | 11.47 |
| ATOM | 12573 | O | LEU | 1880 | 12.573 | −35.430 | 31.231 | 1.00 | 12.83 |
| ATOM | 12574 | N | LEU | 1881 | 13.124 | −33.546 | 32.324 | 1.00 | 8.79 |
| ATOM | 12575 | CA | LEU | 1881 | 12.881 | −32.705 | 31.166 | 1.00 | 10.82 |
| ATOM | 12576 | CB | LEU | 1881 | 11.882 | −31.600 | 31.534 | 1.00 | 11.46 |
| ATOM | 12577 | CG | LEU | 1881 | 11.152 | −30.776 | 30.459 | 1.00 | 14.79 |
| ATOM | 12578 | CD1 | LEU | 1881 | 10.169 | −29.889 | 31.176 | 1.00 | 14.22 |
| ATOM | 12579 | CD2 | LEU | 1881 | 12.096 | −29.933 | 29.599 | 1.00 | 17.18 |
| ATOM | 12580 | C | LEU | 1881 | 14.140 | −32.077 | 30.601 | 1.00 | 11.03 |
| ATOM | 12581 | O | LEU | 1881 | 14.802 | −31.293 | 31.271 | 1.00 | 13.13 |
| ATOM | 12582 | N | LEU | 1882 | 14.460 | −32.434 | 29.362 | 1.00 | 10.50 |
| ATOM | 12583 | CA | LEU | 1882 | 15.622 | −31.885 | 28.698 | 1.00 | 11.79 |
| ATOM | 12584 | CB | LEU | 1882 | 16.333 | −32.952 | 27.868 | 1.00 | 11.43 |
| ATOM | 12585 | CG | LEU | 1882 | 17.203 | −33.973 | 28.607 | 1.00 | 15.97 |
| ATOM | 12586 | CD1 | LEU | 1882 | 16.363 | −34.799 | 29.575 | 1.00 | 17.09 |
| ATOM | 12587 | CD2 | LEU | 1882 | 17.882 | −34.878 | 27.577 | 1.00 | 16.23 |
| ATOM | 12588 | C | LEU | 1882 | 15.146 | −30.767 | 27.787 | 1.00 | 13.92 |
| ATOM | 12589 | O | LEU | 1882 | 14.172 | −30.926 | 27.051 | 1.00 | 17.46 |
| ATOM | 12590 | N | ALA | 1883 | 15.808 | −29.623 | 27.852 | 1.00 | 11.32 |
| ATOM | 12591 | CA | ALA | 1883 | 15.426 | −28.520 | 26.990 | 1.00 | 12.45 |
| ATOM | 12592 | CB | ALA | 1883 | 14.960 | −27.342 | 27.824 | 1.00 | 12.10 |
| ATOM | 12593 | C | ALA | 1883 | 16.624 | −28.132 | 26.144 | 1.00 | 11.52 |
| ATOM | 12594 | O | ALA | 1883 | 17.748 | −28.030 | 26.639 | 1.00 | 10.54 |
| ATOM | 12595 | N | ASP | 1884 | 16.384 | −27.941 | 24.853 | 1.00 | 12.57 |
| ATOM | 12596 | CA | ASP | 1884 | 17.447 | −27.533 | 23.935 | 1.00 | 12.76 |
| ATOM | 12597 | CB | ASP | 1884 | 16.981 | −27.649 | 22.477 | 1.00 | 13.52 |
| ATOM | 12598 | CG | ASP | 1884 | 17.351 | −28.961 | 21.828 | 1.00 | 15.04 |
| ATOM | 12599 | OD1 | ASP | 1884 | 17.887 | −29.851 | 22.513 | 1.00 | 16.51 |
| ATOM | 12600 | OD2 | ASP | 1884 | 17.098 | −29.083 | 20.610 | 1.00 | 14.48 |
| ATOM | 12601 | C | ASP | 1884 | 17.785 | −26.069 | 24.139 | 1.00 | 14.66 |
| ATOM | 12602 | O | ASP | 1884 | 16.934 | −25.277 | 24.550 | 1.00 | 13.13 |
| ATOM | 12603 | N | LEU | 1885 | 19.040 | −25.719 | 23.880 | 1.00 | 12.80 |
| ATOM | 12604 | CA | LEU | 1885 | 19.449 | −24.318 | 23.862 | 1.00 | 12.01 |
| ATOM | 12605 | CB | LEU | 1885 | 20.860 | −24.097 | 24.411 | 1.00 | 11.51 |
| ATOM | 12606 | CG | LEU | 1885 | 20.951 | −24.044 | 25.932 | 1.00 | 13.75 |
| ATOM | 12607 | CD1 | LEU | 1885 | 22.335 | −23.553 | 26.373 | 1.00 | 14.19 |
| ATOM | 12608 | CD2 | LEU | 1885 | 19.862 | −23.123 | 26.460 | 1.00 | 15.95 |
| ATOM | 12609 | C | LEU | 1885 | 19.472 | −24.182 | 22.340 | 1.00 | 10.80 |
| ATOM | 12610 | O | LEU | 1885 | 20.170 | −24.932 | 21.653 | 1.00 | 11.71 |
| ATOM | 12611 | N | PRO | 1886 | 18.696 | −23.245 | 21.793 | 1.00 | 10.45 |
| ATOM | 12612 | CD | PRO | 1886 | 17.852 | −22.290 | 22.538 | 1.00 | 10.25 |
| ATOM | 12613 | CA | PRO | 1886 | 18.606 | −23.014 | 20.350 | 1.00 | 11.23 |
| ATOM | 12614 | CB | PRO | 1886 | 17.356 | −22.158 | 20.234 | 1.00 | 11.55 |
| ATOM | 12615 | CG | PRO | 1886 | 17.457 | −21.296 | 21.474 | 1.00 | 11.59 |
| ATOM | 12616 | C | PRO | 1886 | 19.819 | −22.372 | 19.670 | 1.00 | 11.51 |
| ATOM | 12617 | O | PRO | 1886 | 20.825 | −22.065 | 20.314 | 1.00 | 13.04 |
| ATOM | 12618 | N | PHE | 1887 | 19.692 | −22.178 | 18.358 | 1.00 | 9.35 |
| ATOM | 12619 | CA | PHE | 1887 | 20.723 | −21.578 | 17.516 | 1.00 | 10.68 |
| ATOM | 12620 | CB | PHE | 1887 | 20.125 | −21.260 | 16.136 | 1.00 | 11.05 |
| ATOM | 12621 | CG | PHE | 1887 | 20.992 | −20.370 | 15.277 | 1.00 | 12.47 |
| ATOM | 12622 | CD1 | PHE | 1887 | 22.251 | −20.793 | 14.856 | 1.00 | 15.73 |
| ATOM | 12623 | CD2 | PHE | 1887 | 20.546 | −19.110 | 14.890 | 1.00 | 14.62 |
| ATOM | 12624 | CE1 | PHE | 1887 | 23.058 | −19.975 | 14.057 | 1.00 | 15.72 |
| ATOM | 12625 | CE2 | PHE | 1887 | 21.340 | −18.276 | 14.092 | 1.00 | 13.51 |
| ATOM | 12626 | CZ | PHE | 1887 | 22.598 | −18.717 | 13.679 | 1.00 | 15.84 |
| ATOM | 12627 | C | PHE | 1887 | 21.290 | −20.311 | 18.153 | 1.00 | 10.86 |
| ATOM | 12628 | O | PHE | 1887 | 20.547 | −19.417 | 18.545 | 1.00 | 9.48 |
| ATOM | 12629 | N | MET | 1888 | 22.614 | −20.260 | 18.250 | 1.00 | 8.23 |
| ATOM | 12630 | CA | MET | 1888 | 23.337 | −19.130 | 18.827 | 1.00 | 10.91 |
| ATOM | 12631 | CB | MET | 1888 | 23.230 | −17.906 | 17.906 | 1.00 | 13.53 |
| ATOM | 12632 | CG | MET | 1888 | 24.386 | −16.914 | 18.080 | 1.00 | 15.10 |
| ATOM | 12633 | SD | MET | 1888 | 26.011 | −17.588 | 17.599 | 1.00 | 16.09 |
| ATOM | 12634 | CE | MET | 1888 | 26.079 | −17.055 | 15.901 | 1.00 | 19.82 |
| ATOM | 12635 | C | MET | 1888 | 22.920 | −18.736 | 20.251 | 1.00 | 12.89 |
| ATOM | 12636 | O | MET | 1888 | 23.012 | −17.564 | 20.629 | 1.00 | 13.39 |
| ATOM | 12637 | N | ALA | 1889 | 22.468 | −19.707 | 21.040 | 1.00 | 10.65 |
| ATOM | 12638 | CA | ALA | 1889 | 22.079 | −19.421 | 22.413 | 1.00 | 11.53 |
| ATOM | 12639 | CB | ALA | 1889 | 20.803 | −20.186 | 22.791 | 1.00 | 12.05 |
| ATOM | 12640 | C | ALA | 1889 | 23.200 | −19.776 | 23.383 | 1.00 | 11.33 |
| ATOM | 12641 | O | ALA | 1889 | 23.067 | −19.595 | 24.595 | 1.00 | 13.56 |
| ATOM | 12642 | N | TYR | 1890 | 24.305 | −20.292 | 22.859 | 1.00 | 11.07 |
| ATOM | 12643 | CA | TYR | 1890 | 25.439 | −20.634 | 23.707 | 1.00 | 12.50 |
| ATOM | 12644 | CB | TYR | 1890 | 25.378 | −22.121 | 24.089 | 1.00 | 12.92 |
| ATOM | 12645 | CG | TYR | 1890 | 25.067 | −23.060 | 22.939 | 1.00 | 13.64 |
| ATOM | 12646 | CD1 | TYR | 1890 | 26.089 | −23.716 | 22.253 | 1.00 | 14.95 |
| ATOM | 12647 | CE1 | TYR | 1890 | 25.809 | −24.579 | 21.191 | 1.00 | 15.46 |
| ATOM | 12648 | CD2 | TYR | 1890 | 23.750 | −23.288 | 22.535 | 1.00 | 14.15 |
| ATOM | 12649 | CE2 | TYR | 1890 | 23.456 | −24.150 | 21.471 | 1.00 | 15.84 |
| ATOM | 12650 | CZ | TYR | 1890 | 24.488 | −24.788 | 20.809 | 1.00 | 16.93 |
| ATOM | 12651 | OH | TYR | 1890 | 24.206 | −25.631 | 19.756 | 1.00 | 19.58 |
| ATOM | 12652 | C | TYR | 1890 | 26.743 | −20.287 | 23.000 | 1.00 | 12.88 |
| ATOM | 12653 | O | TYR | 1890 | 27.716 | −21.043 | 23.037 | 1.00 | 13.54 |
| ATOM | 12654 | N | ALA | 1891 | 26.741 | −19.114 | 22.371 | 1.00 | 11.98 |
| ATOM | 12655 | CA | ALA | 1891 | 27.885 | −18.600 | 21.618 | 1.00 | 13.29 |
| ATOM | 12656 | CB | ALA | 1891 | 27.472 | −17.339 | 20.862 | 1.00 | 13.53 |
| ATOM | 12657 | C | ALA | 1891 | 29.100 | −18.305 | 22.490 | 1.00 | 13.87 |
| ATOM | 12658 | O | ALA | 1891 | 30.231 | −18.288 | 22.009 | 1.00 | 13.32 |
| ATOM | 12659 | N | THR | 1892 | 28.850 | −18.059 | 23.769 | 1.00 | 12.24 |
| ATOM | 12660 | CA | THR | 1892 | 29.905 | −17.796 | 24.737 | 1.00 | 12.51 |
| ATOM | 12661 | CB | THR | 1892 | 30.098 | −16.286 | 25.011 | 1.00 | 11.96 |
| ATOM | 12662 | OG1 | THR | 1892 | 28.968 | −15.783 | 25.737 | 1.00 | 12.50 |
| ATOM | 12663 | CG2 | THR | 1892 | 30.235 | −15.516 | 23.709 | 1.00 | 13.36 |
| ATOM | 12664 | C | THR | 1892 | 29.448 | −18.432 | 26.033 | 1.00 | 13.46 |
| ATOM | 12665 | O | THR | 1892 | 28.252 | −18.666 | 26.231 | 1.00 | 14.08 |
| ATOM | 12666 | N | PRO | 1893 | 30.393 | −18.732 | 26.932 | 1.00 | 14.40 |
| ATOM | 12667 | CD | PRO | 1893 | 31.860 | −18.731 | 26.789 | 1.00 | 13.32 |
| ATOM | 12668 | CA | PRO | 1893 | 29.988 | −19.339 | 28.196 | 1.00 | 14.16 |
| ATOM | 12669 | CB | PRO | 1893 | 31.316 | −19.513 | 28.929 | 1.00 | 17.60 |
| ATOM | 12670 | CG | PRO | 1893 | 32.274 | −19.784 | 27.800 | 1.00 | 15.21 |
| ATOM | 12671 | C | PRO | 1893 | 28.992 | −18.442 | 28.936 | 1.00 | 13.95 |
| ATOM | 12672 | O | PRO | 1893 | 27.964 | −18.910 | 29.425 | 1.00 | 11.45 |
| ATOM | 12673 | N | GLU | 1894 | 29.273 | −17.144 | 28.994 | 1.00 | 15.13 |
| ATOM | 12674 | CA | GLU | 1894 | 28.374 | −16.224 | 29.681 | 1.00 | 15.41 |
| ATOM | 12675 | CB | GLU | 1894 | 28.946 | −14.803 | 29.634 | 1.00 | 19.41 |
| ATOM | 12676 | CG | GLU | 1894 | 28.154 | −13.789 | 30.437 | 1.00 | 28.99 |
| ATOM | 12677 | CD | GLU | 1894 | 29.047 | −12.721 | 31.046 | 1.00 | 32.24 |
| ATOM | 12678 | OE1 | GLU | 1894 | 29.945 | −12.223 | 30.334 | 1.00 | 34.50 |
| ATOM | 12679 | OE2 | GLU | 1894 | 28.849 | −12.379 | 32.233 | 1.00 | 37.07 |
| ATOM | 12680 | C | GLU | 1894 | 26.951 | −16.261 | 29.111 | 1.00 | 14.63 |
| ATOM | 12681 | O | GLU | 1894 | 25.981 | −16.257 | 29.869 | 1.00 | 14.06 |
| ATOM | 12682 | N | GLN | 1895 | 26.811 | −16.307 | 27.787 | 1.00 | 13.27 |
| ATOM | 12683 | CA | GLN | 1895 | 25.473 | −16.360 | 27.184 | 1.00 | 14.64 |
| ATOM | 12684 | CB | GLN | 1895 | 25.539 | −16.123 | 25.675 | 1.00 | 18.45 |
| ATOM | 12685 | CG | GLN | 1895 | 26.046 | −14.746 | 25.293 | 1.00 | 26.37 |
| ATOM | 12686 | CD | GLN | 1895 | 25.806 | −14.442 | 23.835 | 1.00 | 30.60 |
| ATOM | 12687 | OE1 | GLN | 1895 | 24.660 | −14.396 | 23.389 | 1.00 | 35.03 |
| ATOM | 12688 | NE2 | GLN | 1895 | 26.883 | −14.236 | 23.077 | 1.00 | 35.29 |
| ATOM | 12689 | C | GLN | 1895 | 24.810 | −17.704 | 27.445 | 1.00 | 14.04 |
| ATOM | 12690 | O | GLN | 1895 | 23.587 | −17.791 | 27.603 | 1.00 | 12.48 |
| ATOM | 12691 | N | ALA | 1896 | 25.616 | −18.758 | 27.465 | 1.00 | 11.38 |
| ATOM | 12692 | CA | ALA | 1896 | 25.085 | −20.086 | 27.732 | 1.00 | 11.76 |
| ATOM | 12693 | CB | ALA | 1896 | 26.188 | −21.149 | 27.572 | 1.00 | 11.03 |
| ATOM | 12694 | C | ALA | 1896 | 24.510 | −20.130 | 29.153 | 1.00 | 11.59 |
| ATOM | 12695 | O | ALA | 1896 | 23.460 | −20.733 | 29.387 | 1.00 | 11.46 |
| ATOM | 12696 | N | PHE | 1897 | 25.191 | −19.475 | 30.094 | 1.00 | 10.77 |
| ATOM | 12697 | CA | PHE | 1897 | 24.738 | −19.446 | 31.485 | 1.00 | 11.53 |
| ATOM | 12698 | CB | PHE | 1897 | 25.690 | −18.639 | 32.390 | 1.00 | 9.99 |
| ATOM | 12699 | CG | PHE | 1897 | 27.119 | −19.116 | 32.392 | 1.00 | 10.28 |
| ATOM | 12700 | CD1 | PHE | 1897 | 27.439 | −20.445 | 32.150 | 1.00 | 12.24 |
| ATOM | 12701 | CD2 | PHE | 1897 | 28.153 | −18.211 | 32.657 | 1.00 | 9.95 |
| ATOM | 12702 | CE1 | PHE | 1897 | 28.775 | −20.879 | 32.166 | 1.00 | 12.67 |
| ATOM | 12703 | CE2 | PHE | 1897 | 29.489 | −18.623 | 32.677 | 1.00 | 11.90 |
| ATOM | 12704 | CZ | PHE | 1897 | 29.799 | −19.959 | 32.429 | 1.00 | 13.96 |
| ATOM | 12705 | C | PHE | 1897 | 23.364 | −18.814 | 31.627 | 1.00 | 11.72 |
| ATOM | 12706 | O | PHE | 1897 | 22.498 | −19.343 | 32.322 | 1.00 | 10.76 |
| ATOM | 12707 | N | GLU | 1898 | 23.183 | −17.667 | 30.986 | 1.00 | 11.55 |
| ATOM | 12708 | CA | GLU | 1898 | 21.924 | −16.945 | 31.060 | 1.00 | 14.41 |
| ATOM | 12709 | CB | GLU | 1898 | 22.072 | −15.550 | 30.444 | 1.00 | 17.46 |
| ATOM | 12710 | CG | GLU | 1898 | 20.906 | −14.623 | 30.790 | 1.00 | 25.46 |
| ATOM | 12711 | CD | GLU | 1898 | 20.594 | −14.618 | 32.284 | 1.00 | 28.92 |
| ATOM | 12712 | OE1 | GLU | 1898 | 21.449 | −14.157 | 33.072 | 1.00 | 31.30 |
| ATOM | 12713 | OE2 | GLU | 1898 | 19.495 | −15.105 | 32.675 | 1.00 | 34.77 |
| ATOM | 12714 | C | GLU | 1898 | 20.793 | −17.692 | 30.382 | 1.00 | 12.46 |
| ATOM | 12715 | O | GLU | 1898 | 19.701 | −17.792 | 30.933 | 1.00 | 11.48 |
| ATOM | 12716 | N | ASN | 1899 | 21.045 | −18.229 | 29.192 | 1.00 | 12.32 |
| ATOM | 12717 | CA | ASN | 1899 | 19.988 | −18.947 | 28.489 | 1.00 | 11.52 |
| ATOM | 12718 | CB | ASN | 1899 | 20.318 | −19.064 | 26.999 | 1.00 | 12.87 |
| ATOM | 12719 | CG | ASN | 1899 | 20.455 | −17.700 | 26.329 | 1.00 | 14.75 |
| ATOM | 12720 | OD1 | ASN | 1899 | 19.690 | −16.777 | 26.621 | 1.00 | 10.94 |
| ATOM | 12721 | ND2 | ASN | 1899 | 21.405 | −17.579 | 25.411 | 1.00 | 11.61 |
| ATOM | 12722 | C | ASN | 1899 | 19.686 | −20.311 | 29.096 | 1.00 | 11.22 |
| ATOM | 12723 | O | ASN | 1899 | 18.534 | −20.758 | 29.053 | 1.00 | 10.29 |
| ATOM | 12724 | N | ALA | 1900 | 20.690 | −20.985 | 29.658 | 1.00 | 10.29 |
| ATOM | 12725 | CA | ALA | 1900 | 20.430 | −22.271 | 30.307 | 1.00 | 9.38 |
| ATOM | 12726 | CB | ALA | 1900 | 21.746 | −22.926 | 30.770 | 1.00 | 8.73 |
| ATOM | 12727 | C | ALA | 1900 | 19.543 | −21.970 | 31.522 | 1.00 | 9.37 |
| ATOM | 12728 | O | ALA | 1900 | 18.551 | −22.646 | 31.762 | 1.00 | 8.92 |
| ATOM | 12729 | N | ALA | 1901 | 19.908 | −20.936 | 32.275 | 1.00 | 10.46 |
| ATOM | 12730 | CA | ALA | 1901 | 19.147 | −20.551 | 33.458 | 1.00 | 9.71 |
| ATOM | 12731 | CB | ALA | 1901 | 19.748 | −19.294 | 34.086 | 1.00 | 9.98 |
| ATOM | 12732 | C | ALA | 1901 | 17.686 | −20.305 | 33.109 | 1.00 | 9.18 |
| ATOM | 12733 | O | ALA | 1901 | 16.783 | −20.733 | 33.823 | 1.00 | 9.21 |
| ATOM | 12734 | N | THR | 1902 | 17.456 | −19.601 | 32.007 | 1.00 | 9.93 |
| ATOM | 12735 | CA | THR | 1902 | 16.092 | −19.293 | 31.588 | 1.00 | 10.15 |
| ATOM | 12736 | CB | THR | 1902 | 16.081 | −28.483 | 30.279 | 1.00 | 10.21 |
| ATOM | 12737 | OG1 | THR | 1902 | 16.814 | −17.268 | 30.484 | 1.00 | 10.06 |
| ATOM | 12738 | CG2 | THR | 1902 | 14.647 | −18.122 | 29.878 | 1.00 | 9.60 |
| ATOM | 12739 | C | THR | 1902 | 15.264 | −20.549 | 31.403 | 1.00 | 10.35 |
| ATOM | 12740 | O | THR | 1902 | 14.156 | −20.659 | 31.941 | 1.00 | 8.19 |
| ATOM | 12741 | N | VAL | 1903 | 15.801 | −21.506 | 30.655 | 1.00 | 10.07 |
| ATOM | 12742 | CA | VAL | 1903 | 15.052 | −22.723 | 30.410 | 1.00 | 12.02 |
| ATOM | 12743 | CB | VAL | 1903 | 15.620 | −23.470 | 29.168 | 1.00 | 13.59 |
| ATOM | 12744 | CG1 | VAL | 1903 | 16.916 | −24.192 | 29.494 | 1.00 | 12.92 |
| ATOM | 12745 | CG2 | VAL | 1903 | 14.577 | −24.403 | 28.622 | 1.00 | 21.31 |
| ATOM | 12746 | C | VAL | 1903 | 14.936 | −23.627 | 31.648 | 1.00 | 10.38 |
| ATOM | 12747 | O | VAL | 1903 | 13.961 | −24.369 | 31.787 | 1.00 | 9.04 |
| ATOM | 12748 | N | MET | 1904 | 15.901 | −23.545 | 32.558 | 1.00 | 10.20 |
| ATOM | 12749 | CA | MET | 1904 | 15.842 | −24.351 | 33.778 | 1.00 | 11.10 |
| ATOM | 12750 | CB | MET | 1904 | 17.223 | −24.428 | 34.444 | 1.00 | 12.49 |
| ATOM | 12751 | CG | MET | 1904 | 18.307 | −25.051 | 33.599 | 1.00 | 18.01 |
| ATOM | 12752 | SD | MET | 1904 | 18.226 | −26.845 | 33.611 | 1.00 | 19.52 |
| ATOM | 12753 | CE | MET | 1904 | 18.566 | −27.204 | 35.406 | 1.00 | 18.10 |
| ATOM | 12754 | C | MET | 1904 | 14.826 | −23.742 | 34.761 | 1.00 | 9.98 |
| ATOM | 12755 | O | MET | 1904 | 14.108 | −24.479 | 35.433 | 1.00 | 11.43 |
| ATOM | 12756 | N | ARG | 1905 | 14.770 | −22.412 | 34.860 | 1.00 | 8.90 |
| ATOM | 12757 | CA | ARG | 1905 | 13.799 | −21.809 | 35.775 | 1.00 | 8.03 |
| ATOM | 12758 | CB | ARG | 1905 | 13.982 | −20.295 | 35.901 | 1.00 | 8.24 |
| ATOM | 12759 | CG | ARG | 1905 | 15.331 | −19.885 | 36.473 | 1.00 | 12.25 |
| ATOM | 12760 | CD | ARG | 1905 | 15.304 | −18.465 | 37.003 | 1.00 | 11.94 |
| ATOM | 12761 | NE | ARG | 1905 | 16.652 | −17.990 | 37.289 | 1.00 | 15.25 |
| ATOM | 12762 | CZ | ARG | 1905 | 17.447 | −17.418 | 36.393 | 1.00 | 13.90 |
| ATOM | 12763 | NH1 | ARG | 1905 | 17.029 | −17.233 | 35.149 | 1.00 | 14.60 |
| ATOM | 12764 | NH2 | ARG | 1905 | 18.677 | −17.063 | 36.733 | 1.00 | 17.42 |
| ATOM | 12765 | C | ARG | 1905 | 12.398 | −22.099 | 35.256 | 1.00 | 9.53 |
| ATOM | 12766 | O | ARG | 1905 | 11.445 | −22.195 | 36.024 | 1.00 | 10.47 |
| ATOM | 12767 | N | ALA | 1906 | 12.291 | −22.274 | 33.946 | 1.00 | 8.58 |
| ATOM | 12768 | CA | ALA | 1906 | 11.011 | −22.538 | 33.317 | 1.00 | 8.67 |
| ATOM | 12769 | CB | ALA | 1906 | 11.100 | −22.235 | 31.814 | 1.00 | 9.41 |
| ATOM | 12770 | C | ALA | 1906 | 10.511 | −23.963 | 33.535 | 1.00 | 10.77 |
| ATOM | 12771 | O | ALA | 1906 | 9.351 | −24.270 | 33.250 | 1.00 | 10.28 |
| ATOM | 12772 | N | GLY | 1907 | 11.382 | −24.841 | 34.018 | 1.00 | 10.02 |
| ATOM | 12773 | CA | GLY | 1907 | 10.962 | −26.209 | 34.291 | 1.00 | 9.93 |
| ATOM | 12774 | C | GLY | 1907 | 11.889 | −27.330 | 33.869 | 1.00 | 12.15 |
| ATOM | 12775 | O | GLY | 1907 | 11.684 | −28.485 | 34.261 | 1.00 | 11.23 |
| ATOM | 12776 | N | ALA | 1908 | 12.907 | −27.009 | 33.081 | 1.00 | 11.34 |
| ATOM | 12777 | CA | ALA | 1908 | 13.851 | −28.026 | 32.612 | 1.00 | 11.08 |
| ATOM | 12778 | CB | ALA | 1908 | 14.702 | −27.455 | 31.475 | 1.00 | 10.75 |
| ATOM | 12779 | C | ALA | 1908 | 14.758 | −28.529 | 33.739 | 1.00 | 11.38 |
| ATOM | 12780 | O | ALA | 1908 | 14.955 | −27.848 | 34.747 | 1.00 | 8.45 |
| ATOM | 12781 | N | ASN | 1909 | 15.303 | −29.729 | 33.567 | 1.00 | 11.47 |
| ATOM | 12782 | CA | ASN | 1909 | 16.205 | −30.302 | 34.564 | 1.00 | 10.53 |
| ATOM | 12783 | CB | ASN | 1909 | 15.804 | −31.727 | 34.939 | 1.00 | 12.92 |
| ATOM | 12784 | CG | ASN | 1909 | 14.432 | −31.812 | 35.556 | 1.00 | 14.93 |
| ATOM | 12785 | OD1 | ASN | 1909 | 14.140 | −31.192 | 36.589 | 1.00 | 14.28 |
| ATOM | 12786 | ND2 | ASN | 1909 | 13.577 | −32.595 | 34.928 | 1.00 | 10.46 |
| ATOM | 12787 | C | ASN | 1909 | 17.593 | −30.385 | 33.970 | 1.00 | 11.82 |
| ATOM | 12788 | O | ASN | 1909 | 18.569 | −30.560 | 34.682 | 1.00 | 12.44 |
| ATOM | 12789 | N | MET | 1910 | 17.670 | −30.262 | 32.653 | 1.00 | 10.66 |
| ATOM | 12790 | CA | MET | 1910 | 18.934 | −30.391 | 31.960 | 1.00 | 11.23 |
| ATOM | 12791 | CB | MET | 1910 | 19.228 | −31.888 | 31.799 | 1.00 | 11.33 |
| ATOM | 12792 | CG | MET | 1910 | 20.340 | −32.264 | 30.850 | 1.00 | 15.81 |
| ATOM | 12793 | SD | MET | 1910 | 20.549 | −34.076 | 30.827 | 1.00 | 17.80 |
| ATOM | 12794 | CE | MET | 1910 | 21.887 | −34.311 | 32.027 | 1.00 | 18.58 |
| ATOM | 12795 | C | MET | 1910 | 18.822 | −29.716 | 30.601 | 1.00 | 10.32 |
| ATOM | 12796 | O | MET | 1910 | 17.730 | −29.632 | 30.036 | 1.00 | 10.64 |
| ATOM | 12797 | N | VAL | 1911 | 19.941 | −29.223 | 30.086 | 1.00 | 9.77 |
| ATOM | 12798 | CA | VAL | 1911 | 19.942 | −28.561 | 28.791 | 1.00 | 10.49 |
| ATOM | 12799 | CB | VAL | 1911 | 20.552 | −27.149 | 28.904 | 1.00 | 14.32 |
| ATOM | 12800 | CG1 | VAL | 1911 | 20.525 | −26.477 | 27.563 | 1.00 | 20.77 |
| ATOM | 12801 | CG2 | VAL | 1911 | 19.784 | −26.322 | 29.924 | 1.00 | 16.29 |
| ATOM | 12802 | C | VAL | 1911 | 20.752 | −29.368 | 27.782 | 1.00 | 11.18 |
| ATOM | 12803 | O | VAL | 1911 | 21.758 | −29.989 | 28.128 | 1.00 | 12.97 |
| ATOM | 12804 | N | LYS | 1912 | 20.304 | −29.364 | 26.535 | 1.00 | 11.26 |
| ATOM | 12805 | CA | LYS | 1912 | 21.019 | −30.062 | 25.480 | 1.00 | 11.63 |
| ATOM | 12806 | CB | LYS | 1912 | 20.088 | −30.999 | 24.712 | 1.00 | 11.49 |
| ATOM | 12807 | CG | LYS | 1912 | 20.806 | −31.774 | 23.598 | 1.00 | 13.76 |
| ATOM | 12808 | CD | LYS | 1912 | 20.027 | −33.015 | 23.160 | 1.00 | 15.10 |
| ATOM | 12809 | CE | LYS | 1912 | 18.742 | −32.667 | 22.415 | 1.00 | 17.50 |
| ATOM | 12810 | NZ | LYS | 1912 | 18.980 | −32.003 | 21.096 | 1.00 | 16.50 |
| ATOM | 12811 | C | LYS | 1912 | 21.607 | −29.027 | 24.522 | 1.00 | 10.58 |
| ATOM | 12812 | O | LYS | 1912 | 20.928 | −28.077 | 24.128 | 1.00 | 12.27 |
| ATOM | 12813 | N | ILE | 1913 | 22.875 | −29.208 | 24.169 | 1.00 | 11.18 |
| ATOM | 12814 | CA | ILE | 1913 | 23.568 | −28.301 | 23.260 | 1.00 | 14.22 |
| ATOM | 12815 | CB | ILE | 1913 | 24.541 | −27.375 | 24.034 | 1.00 | 18.27 |
| ATOM | 12816 | CG2 | ILE | 1913 | 23.738 | −26.374 | 24.883 | 1.00 | 15.84 |
| ATOM | 12817 | CG1 | ILE | 1913 | 25.463 | −28.206 | 24.925 | 1.00 | 18.46 |
| ATOM | 12818 | CD1 | ILE | 1913 | 26.415 | −27.376 | 25.786 | 1.00 | 18.67 |
| ATOM | 12819 | C | ILE | 1913 | 24.335 | −29.121 | 22.228 | 1.00 | 15.17 |
| ATOM | 12820 | O | ILE | 1913 | 24.929 | −30.149 | 22.583 | 1.00 | 15.09 |
| ATOM | 12821 | N | GLU | 1914 | 24.328 | −28.671 | 20.975 | 1.00 | 15.40 |
| ATOM | 12822 | CA | GLU | 1914 | 25.003 | −29.392 | 19.889 | 1.00 | 18.44 |
| ATOM | 12823 | CB | GLU | 1914 | 24.205 | −29.272 | 18.582 | 1.00 | 19.74 |
| ATOM | 12824 | CG | GLU | 1914 | 22.709 | −29.426 | 18.725 | 1.00 | 25.29 |
| ATOM | 12825 | CD | GLU | 1914 | 21.987 | −29.391 | 17.387 | 1.00 | 28.05 |
| ATOM | 12826 | OE1 | GLU | 1914 | 22.415 | −28.626 | 16.497 | 1.00 | 28.95 |
| ATOM | 12827 | OE2 | GLU | 1914 | 20.987 | −30.120 | 17.231 | 1.00 | 31.69 |
| ATOM | 12828 | C | GLU | 1914 | 26.411 | −28.893 | 19.623 | 1.00 | 15.88 |
| ATOM | 12829 | O | GLU | 1914 | 26.630 | −27.696 | 19.463 | 1.00 | 14.70 |
| ATOM | 12830 | N | GLY | 1915 | 27.371 | −29.805 | 19.550 | 1.00 | 16.86 |
| ATOM | 12831 | CA | GLY | 1915 | 28.736 | −29.379 | 19.288 | 1.00 | 18.17 |
| ATOM | 12832 | C | GLY | 1915 | 29.783 | −30.355 | 19.788 | 1.00 | 20.08 |
| ATOM | 12833 | O | GLY | 1915 | 29.497 | −31.203 | 20.831 | 1.00 | 21.39 |
| ATOM | 12834 | N | GLY | 1916 | 30.999 | −30.223 | 19.264 | 1.00 | 20.87 |
| ATOM | 12835 | CA | GLY | 1918 | 32.086 | −31.091 | 19.682 | 1.00 | 22.00 |
| ATOM | 12838 | C | GLY | 1916 | 32.949 | −30.518 | 20.768 | 1.00 | 22.33 |
| ATOM | 12837 | O | GLY | 1916 | 32.449 | −29.886 | 21.705 | 1.00 | 21.23 |
| ATOM | 12838 | N | GLU | 1917 | 34.258 | −30.720 | 20.656 | 1.00 | 22.98 |
| ATOM | 12839 | CA | GLU | 1917 | 35.178 | −30.232 | 21.677 | 1.00 | 22.46 |
| ATOM | 12840 | CB | GLU | 1917 | 36.608 | −30.681 | 21.376 | 1.00 | 26.72 |
| ATOM | 12841 | CG | GLU | 1917 | 36.939 | −32.014 | 22.003 | 1.00 | 33.75 |
| ATOM | 12842 | CD | GLU | 1917 | 38.367 | −32.087 | 22.494 | 1.00 | 34.57 |
| ATOM | 12843 | OE1 | GLU | 1917 | 38.778 | −31.190 | 23.257 | 1.00 | 37.45 |
| ATOM | 12844 | OE2 | GLU | 1917 | 39.087 | −33.048 | 22.120 | 1.00 | 39.08 |
| ATOM | 12845 | C | GLU | 1917 | 35.189 | −28.738 | 21.944 | 1.00 | 19.95 |
| ATOM | 12846 | O | GLU | 1917 | 35.604 | −28.324 | 23.018 | 1.00 | 18.13 |
| ATOM | 12847 | N | TRP | 1918 | 34.747 | −27.920 | 20.994 | 1.00 | 18.83 |
| ATOM | 12848 | CA | TRP | 1918 | 34.771 | −28.492 | 21.255 | 1.00 | 18.44 |
| ATOM | 12849 | CB | TRP | 1918 | 34.372 | −25.880 | 20.010 | 1.00 | 18.23 |
| ATOM | 12850 | CG | TRP | 1918 | 32.918 | −25.705 | 19.630 | 1.00 | 15.75 |
| ATOM | 12851 | CD2 | TRP | 1918 | 31.940 | −24.702 | 19.927 | 1.00 | 15.59 |
| ATOM | 12852 | CE2 | TRP | 1918 | 30.728 | −25.106 | 19.323 | 1.00 | 18.90 |
| ATOM | 12853 | CE3 | TRP | 1918 | 31.970 | −23.499 | 20.646 | 1.00 | 17.10 |
| ATOM | 12854 | CD1 | TRP | 1918 | 32.275 | −28.655 | 18.882 | 1.00 | 18.69 |
| ATOM | 12855 | NE1 | TRP | 1918 | 30.960 | −28.300 | 18.691 | 1.00 | 17.49 |
| ATOM | 12856 | CZ2 | TRP | 1918 | 29.554 | −24.345 | 19.412 | 1.00 | 14.08 |
| ATOM | 12857 | CZ3 | TRP | 1918 | 30.802 | −22.739 | 20.738 | 1.00 | 17.47 |
| ATOM | 12858 | CH2 | TRP | 1918 | 29.607 | −23.170 | 20.121 | 1.00 | 17.12 |
| ATOM | 12859 | C | TRP | 1918 | 33.878 | −28.130 | 22.439 | 1.00 | 15.03 |
| ATOM | 12880 | O | TRP | 1918 | 34.044 | −25.078 | 23.051 | 1.00 | 15.31 |
| ATOM | 12881 | N | LEU | 1919 | 32.944 | −27.013 | 22.778 | 1.00 | 14.71 |
| ATOM | 12862 | CA | LEU | 1919 | 32.021 | −28.761 | 23.891 | 1.00 | 14.78 |
| ATOM | 12863 | CB | LEU | 1919 | 30.882 | −27.454 | 23.626 | 1.00 | 15.37 |
| ATOM | 12864 | CG | LEU | 1919 | 29.808 | −28.886 | 22.519 | 1.00 | 17.11 |
| ATOM | 12885 | CD1 | LEU | 1919 | 28.579 | −27.749 | 22.330 | 1.00 | 18.09 |
| ATOM | 12866 | CD2 | LEU | 1919 | 29.400 | −25.442 | 22.894 | 1.00 | 16.88 |
| ATOM | 12867 | C | LEU | 1919 | 32.530 | −27.206 | 25.259 | 1.00 | 14.85 |
| ATOM | 12868 | O | LEU | 1919 | 31.896 | −26.928 | 26.270 | 1.00 | 12.57 |
| ATOM | 12869 | N | VAL | 1920 | 33.667 | −27.893 | 25.298 | 1.00 | 15.61 |
| ATOM | 12870 | CA | VAL | 1920 | 34.189 | −28.386 | 26.589 | 1.00 | 15.42 |
| ATOM | 12871 | CB | VAL | 1920 | 35.582 | −29.008 | 28.379 | 1.00 | 18.95 |
| ATOM | 12872 | CG1 | VAL | 1920 | 36.302 | −29.125 | 27.717 | 1.00 | 18.07 |
| ATOM | 12873 | CG2 | VAL | 1920 | 35.425 | −30.389 | 25.764 | 1.00 | 15.13 |
| ATOM | 12874 | C | VAL | 1920 | 34.216 | −27.368 | 27.707 | 1.00 | 16.07 |
| ATOM | 12875 | O | VAL | 1920 | 33.758 | −27.662 | 28.808 | 1.00 | 15.60 |
| ATOM | 12878 | N | GLU | 1921 | 34.737 | −26.172 | 27.448 | 1.00 | 15.79 |
| ATOM | 12877 | CA | GLU | 1921 | 34.785 | −25.141 | 28.483 | 1.00 | 18.09 |
| ATOM | 12878 | CB | GLU | 1921 | 35.480 | −23.888 | 27.949 | 1.00 | 18.74 |
| ATOM | 12879 | CG | GLU | 1921 | 35.428 | −22.701 | 28.889 | 1.00 | 24.44 |
| ATOM | 12880 | CD | GLU | 1921 | 36.271 | −21.543 | 28.394 | 1.00 | 27.12 |
| ATOM | 12881 | OE1 | GLU | 1921 | 36.105 | −21.136 | 27.224 | 1.00 | 28.89 |
| ATOM | 12882 | OE2 | GLU | 1921 | 37.104 | −21.045 | 29.177 | 1.00 | 30.38 |
| ATOM | 12883 | C | GLU | 1921 | 33.373 | −24.780 | 28.935 | 1.00 | 15.64 |
| ATOM | 12884 | O | GLU | 1921 | 33.095 | −24.650 | 30.128 | 1.00 | 14.95 |
| ATOM | 12885 | N | THR | 1922 | 32.478 | −24.623 | 27.971 | 1.00 | 16.48 |
| ATOM | 12886 | CA | THR | 1922 | 31.102 | −24.268 | 28.289 | 1.00 | 15.13 |
| ATOM | 12887 | CB | THR | 1922 | 30.285 | −24.074 | 27.017 | 1.00 | 14.39 |
| ATOM | 12888 | OG1 | THR | 1922 | 30.846 | −22.993 | 26.265 | 1.00 | 14.42 |
| ATOM | 12889 | CG2 | THR | 1922 | 28.834 | −23.753 | 27.354 | 1.00 | 13.35 |
| ATOM | 12890 | C | THR | 1922 | 30.439 | −25.321 | 29.161 | 1.00 | 23.15 |
| ATOM | 12891 | O | THR | 1922 | 29.759 | −24.990 | 30.136 | 1.00 | 13.84 |
| ATOM | 12892 | N | VAL | 1923 | 30.632 | −26.589 | 28.815 | 1.00 | 14.02 |
| ATOM | 12893 | CA | VAL | 1923 | 30.042 | −27.671 | 29.590 | 1.00 | 13.28 |
| ATOM | 12894 | CB | VAL | 1923 | 30.285 | −29.039 | 28.911 | 1.00 | 13.90 |
| ATOM | 12895 | CG1 | VAL | 1923 | 29.800 | −30.171 | 29.818 | 1.00 | 14.64 |
| ATOM | 12896 | CG2 | VAL | 1923 | 29.552 | −29.080 | 27.584 | 1.00 | 14.39 |
| ATOM | 12897 | C | VAL | 1923 | 30.611 | −27.687 | 31.000 | 1.00 | 15.48 |
| ATOM | 12898 | O | VAL | 1923 | 29.883 | −27.755 | 31.982 | 1.00 | 12.62 |
| ATOM | 12899 | N | GLN | 1924 | 31.932 | −27.598 | 31.109 | 1.00 | 15.37 |
| ATOM | 12900 | CA | GLN | 1924 | 32.566 | −27.606 | 32.434 | 1.00 | 17.23 |
| ATOM | 12901 | CB | GLN | 1924 | 34.087 | −27.396 | 32.310 | 1.00 | 19.46 |
| ATOM | 12902 | CG | GLN | 1924 | 34.796 | −28.434 | 31.437 | 1.00 | 23.92 |
| ATOM | 12903 | CD | GLN | 1924 | 36.306 | −28.223 | 31.344 | 1.00 | 28.58 |
| ATOM | 12904 | OE1 | GLN | 1924 | 36.778 | −27.120 | 31.054 | 1.00 | 27.54 |
| ATOM | 12905 | NE2 | GLN | 1924 | 37.069 | −29.293 | 31.575 | 1.00 | 28.43 |
| ATOM | 12906 | C | GLN | 1924 | 31.966 | −26.527 | 33.344 | 1.00 | 16.21 |
| ATOM | 12907 | O | GLN | 1924 | 31.576 | −26.811 | 34.470 | 1.00 | 15.27 |
| ATOM | 12908 | N | MET | 1925 | 31.881 | −25.299 | 32.843 | 1.00 | 15.24 |
| ATOM | 12909 | CA | MET | 1925 | 31.356 | −24.188 | 33.624 | 1.00 | 14.73 |
| ATOM | 12910 | CB | MET | 1925 | 31.688 | −22.875 | 32.921 | 1.00 | 17.14 |
| ATOM | 12911 | CG | MET | 1925 | 33.189 | −22.574 | 32.914 | 1.00 | 17.57 |
| ATOM | 12912 | SD | MET | 1925 | 33.579 | −21.081 | 31.980 | 1.00 | 19.95 |
| ATOM | 12913 | CE | MET | 1925 | 33.416 | −19.842 | 33.296 | 1.00 | 22.55 |
| ATOM | 12914 | C | MET | 1925 | 29.872 | −24.267 | 33.944 | 1.00 | 15.19 |
| ATOM | 12915 | O | MET | 1925 | 29.452 | −23.902 | 35.044 | 1.00 | 12.00 |
| ATOM | 12916 | N | LEU | 1926 | 29.070 | −24.735 | 32.995 | 1.00 | 13.48 |
| ATOM | 12917 | CA | LEU | 1926 | 27.638 | −24.868 | 33.240 | 1.00 | 14.38 |
| ATOM | 12918 | CB | LEU | 1926 | 26.934 | −25.398 | 31.989 | 1.00 | 12.37 |
| ATOM | 12919 | CG | LEU | 1926 | 26.531 | −24.365 | 30.944 | 1.00 | 10.67 |
| ATOM | 12920 | CD1 | LEU | 1926 | 26.209 | −25.077 | 29.631 | 1.00 | 10.46 |
| ATOM | 12921 | CD2 | LEU | 1926 | 25.320 | −23.570 | 31.450 | 1.00 | 11.83 |
| ATOM | 12922 | C | LEU | 1926 | 27.389 | −25.827 | 34.391 | 1.00 | 15.78 |
| ATOM | 12923 | O | LEU | 1926 | 26.604 | −25.542 | 35.298 | 1.00 | 14.90 |
| ATOM | 12924 | N | THR | 1927 | 28.061 | −26.974 | 34.348 | 1.00 | 17.82 |
| ATOM | 12925 | CA | THR | 1927 | 27.891 | −27.988 | 35.385 | 1.00 | 19.33 |
| ATOM | 12926 | CB | THR | 1927 | 28.736 | −29.236 | 35.074 | 1.00 | 22.11 |
| ATOM | 12927 | OG1 | THR | 1927 | 28.239 | −29.852 | 33.873 | 1.00 | 23.96 |
| ATOM | 12928 | CG2 | THR | 1927 | 28.653 | −30.240 | 36.219 | 1.00 | 26.37 |
| ATOM | 12929 | C | THR | 1927 | 28.222 | −27.472 | 36.780 | 1.00 | 20.67 |
| ATOM | 12930 | O | THR | 1927 | 27.482 | −27.738 | 37.731 | 1.00 | 19.83 |
| ATOM | 12931 | N | GLU | 1928 | 29.325 | −26.741 | 36.926 | 1.00 | 19.57 |
| ATOM | 12932 | CA | GLU | 1928 | 29.639 | −26.217 | 38.254 | 1.00 | 20.59 |
| ATOM | 12933 | CB | GLU | 1928 | 31.087 | −25.716 | 38.340 | 1.00 | 23.01 |
| ATOM | 12934 | CG | GLU | 1928 | 31.562 | −24.901 | 37.176 | 1.00 | 24.98 |
| ATOM | 12935 | CD | GLU | 1928 | 32.983 | −24.379 | 37.372 | 1.00 | 25.34 |
| ATOM | 12936 | OE1 | GLU | 1928 | 33.819 | −25.097 | 37.963 | 1.00 | 28.59 |
| ATOM | 12937 | OE2 | GLU | 1928 | 33.271 | −23.258 | 36.920 | 1.00 | 24.69 |
| ATOM | 12938 | C | GLU | 1928 | 28.648 | −25.121 | 38.649 | 1.00 | 19.64 |
| ATOM | 12939 | O | GLU | 1928 | 28.514 | −24.801 | 39.830 | 1.00 | 20.05 |
| ATOM | 12940 | N | ARG | 1929 | 27.943 | −24.558 | 37.666 | 1.00 | 16.54 |
| ATOM | 12941 | CA | ARG | 1929 | 26.938 | −23.536 | 37.948 | 1.00 | 15.92 |
| ATOM | 12942 | CB | ARG | 1929 | 27.001 | −22.425 | 36.901 | 1.00 | 15.43 |
| ATOM | 12943 | CG | ARG | 1929 | 28.222 | −21.543 | 37.095 | 1.00 | 16.86 |
| ATOM | 12944 | CD | ARG | 1929 | 28.530 | −20.685 | 35.884 | 1.00 | 16.91 |
| ATOM | 12945 | NE | ARG | 1929 | 29.786 | −19.956 | 36.071 | 1.00 | 15.63 |
| ATOM | 12946 | CZ | ARG | 1929 | 30.972 | −20.533 | 36.258 | 1.00 | 17.47 |
| ATOM | 12947 | NH1 | ARG | 1929 | 31.080 | −21.855 | 36.284 | 1.00 | 20.43 |
| ATOM | 12948 | NH2 | ARG | 1929 | 32.056 | −19.786 | 36.418 | 1.00 | 17.19 |
| ATOM | 12949 | C | ARG | 1929 | 25.531 | −24.134 | 38.032 | 1.00 | 16.03 |
| ATOM | 12950 | O | ARG | 1929 | 24.528 | −23.480 | 37.724 | 1.00 | 15.35 |
| ATOM | 12951 | N | ALA | 1930 | 25.492 | −25.398 | 38.446 | 1.00 | 15.30 |
| ATOM | 12952 | CA | ALA | 1930 | 24.262 | −26.152 | 38.674 | 1.00 | 14.02 |
| ATOM | 12953 | CB | ALA | 1930 | 23.364 | −25.373 | 39.645 | 1.00 | 17.52 |
| ATOM | 12954 | C | ALA | 1930 | 23.437 | −26.610 | 37.473 | 1.00 | 14.90 |
| ATOM | 12955 | O | ALA | 1930 | 22.302 | −27.051 | 37.655 | 1.00 | 12.87 |
| ATOM | 12956 | N | VAL | 1931 | 23.974 | −26.510 | 36.260 | 1.00 | 13.13 |
| ATOM | 12957 | CA | VAL | 1931 | 23.217 | −26.948 | 35.100 | 1.00 | 13.22 |
| ATOM | 12958 | CB | VAL | 1931 | 23.220 | −25.876 | 33.974 | 1.00 | 14.49 |
| ATOM | 12959 | CG1 | VAL | 1931 | 22.325 | −26.328 | 32.829 | 1.00 | 11.71 |
| ATOM | 12960 | CG2 | VAL | 1931 | 22.746 | −24.539 | 34.522 | 1.00 | 14.16 |
| ATOM | 12961 | C | VAL | 1931 | 23.752 | −28.239 | 34.492 | 1.00 | 13.32 |
| ATOM | 12962 | O | VAL | 1931 | 24.905 | −28.296 | 34.046 | 1.00 | 13.87 |
| ATOM | 12963 | N | PRO | 1932 | 22.940 | −29.307 | 34.502 | 1.00 | 13.80 |
| ATOM | 12964 | CD | PRO | 1932 | 21.715 | −29.508 | 35.290 | 1.00 | 14.21 |
| ATOM | 12965 | CA | PRO | 1932 | 23.395 | −30.567 | 33.908 | 1.00 | 13.32 |
| ATOM | 12966 | CB | PRO | 1932 | 22.396 | −31.596 | 34.437 | 1.00 | 15.25 |
| ATOM | 12967 | CG | PRO | 1932 | 21.178 | −30.791 | 34.712 | 1.00 | 20.66 |
| ATOM | 12968 | C | PRO | 1932 | 23.310 | −30.391 | 32.396 | 1.00 | 11.97 |
| ATOM | 12969 | O | PRO | 1932 | 22.413 | −29.713 | 31.897 | 1.00 | 12.08 |
| ATOM | 12970 | N | VAL | 1933 | 24.228 | −31.012 | 31.669 | 1.00 | 11.35 |
| ATOM | 12971 | CA | VAL | 1933 | 24.270 | −30.869 | 30.218 | 1.00 | 11.25 |
| ATOM | 12972 | CB | VAL | 1933 | 25.577 | −30.149 | 29.793 | 1.00 | 12.56 |
| ATOM | 12973 | CG1 | VAL | 1933 | 25.676 | −30.079 | 28.280 | 1.00 | 10.86 |
| ATOM | 12974 | CG2 | VAL | 1933 | 25.634 | −28.768 | 30.411 | 1.00 | 10.60 |
| ATOM | 12975 | C | VAL | 1933 | 24.205 | −32.194 | 29.474 | 1.00 | 13.16 |
| ATOM | 12976 | O | VAL | 1933 | 24.850 | −33.170 | 29.853 | 1.00 | 12.41 |
| ATOM | 12977 | N | CYS | 1934 | 23.417 | −32.218 | 28.411 | 1.00 | 11.26 |
| ATOM | 12978 | CA | CYS | 1934 | 23.298 | −33.393 | 27.574 | 1.00 | 11.02 |
| ATOM | 12979 | CB | CYS | 1934 | 21.822 | −33.687 | 27.271 | 1.00 | 11.90 |
| ATOM | 12980 | SG | CYS | 1934 | 21.573 | −35.022 | 26.038 | 1.00 | 13.45 |
| ATOM | 12981 | C | CYS | 1934 | 24.031 | −32.993 | 26.301 | 1.00 | 13.27 |
| ATOM | 12982 | O | CYS | 1934 | 23.773 | −31.929 | 25.739 | 1.00 | 12.86 |
| ATOM | 12983 | N | GLY | 1935 | 24.978 | −33.817 | 25.872 | 1.00 | 12.09 |
| ATOM | 12984 | CA | GLY | 1935 | 25.705 | −33.515 | 24.655 | 1.00 | 10.39 |
| ATOM | 12985 | C | GLY | 1935 | 24.870 | −33.961 | 23.475 | 1.00 | 11.98 |
| ATOM | 12986 | O | GLY | 1935 | 23.843 | −34.627 | 23.654 | 1.00 | 12.33 |
| ATOM | 12987 | N | HIS | 1936 | 25.305 | −33.601 | 22.272 | 1.00 | 10.95 |
| ATOM | 12988 | CA | HIS | 1936 | 24.585 | −33.957 | 21.054 | 1.00 | 13.54 |
| ATOM | 12989 | CB | HIS | 1936 | 23.453 | −32.945 | 20.813 | 1.00 | 13.71 |
| ATOM | 12990 | CG | HIS | 1936 | 22.547 | −33.285 | 19.669 | 1.00 | 16.35 |
| ATOM | 12991 | CD2 | HIS | 1936 | 22.722 | −34.088 | 18.593 | 1.00 | 17.45 |
| ATOM | 12992 | ND1 | HIS | 1936 | 21.297 | −32.719 | 19.522 | 1.00 | 13.36 |
| ATOM | 12993 | CE1 | HIS | 1936 | 20.745 | −33.155 | 18.406 | 1.00 | 17.41 |
| ATOM | 12994 | NE2 | HIS | 1936 | 21.589 | −33.986 | 17.821 | 1.00 | 19.02 |
| ATOM | 12995 | C | HIS | 1936 | 25.568 | −33.974 | 19.889 | 1.00 | 12.21 |
| ATOM | 12996 | O | HIS | 1936 | 26.069 | −32.929 | 19.460 | 1.00 | 12.99 |
| ATOM | 12997 | N | LEU | 1937 | 25.825 | −35.176 | 19.382 | 1.00 | 13.16 |
| ATOM | 12998 | CA | LEU | 1937 | 26.767 | −35.384 | 18.287 | 1.00 | 12.94 |
| ATOM | 12999 | CB | LEU | 1937 | 27.994 | −36.157 | 18.797 | 1.00 | 12.68 |
| ATOM | 13000 | CG | LEU | 1937 | 28.833 | −35.486 | 19.884 | 1.00 | 13.53 |
| ATOM | 13001 | CD1 | LEU | 1937 | 29.914 | −36.448 | 20.395 | 1.00 | 15.18 |
| ATOM | 13002 | CD2 | LEU | 1937 | 29.449 | −34.228 | 19.334 | 1.00 | 12.64 |
| ATOM | 13003 | C | LEU | 1937 | 26.141 | −36.137 | 17.126 | 1.00 | 14.83 |
| ATOM | 13004 | O | LEU | 1937 | 25.103 | −36.785 | 17.274 | 1.00 | 12.98 |
| ATOM | 13005 | N | GLY | 1938 | 26.792 | −36.055 | 15.972 | 1.00 | 16.63 |
| ATOM | 13006 | CA | GLY | 1938 | 26.283 | −36.719 | 14.790 | 1.00 | 22.86 |
| ATOM | 13007 | C | GLY | 1938 | 25.712 | −35.675 | 13.854 | 1.00 | 27.34 |
| ATOM | 13008 | O | GLY | 1938 | 26.411 | −34.740 | 13.466 | 1.00 | 27.92 |
| ATOM | 13009 | N | LEU | 1939 | 24.438 | −35.824 | 13.504 | 1.00 | 30.73 |
| ATOM | 13010 | CA | LEU | 1939 | 23.766 | −34.889 | 12.611 | 1.00 | 34.21 |
| ATOM | 13011 | CB | LEU | 1939 | 22.714 | −35.638 | 11.786 | 1.00 | 35.61 |
| ATOM | 13012 | CG | LEU | 1939 | 22.029 | −34.928 | 10.615 | 1.00 | 37.63 |
| ATOM | 13013 | CD1 | LEU | 1939 | 21.406 | −35.969 | 9.702 | 1.00 | 38.32 |
| ATOM | 13014 | CD2 | LEU | 1939 | 20.973 | −33.955 | 11.127 | 1.00 | 38.51 |
| ATOM | 13015 | C | LEU | 1939 | 23.116 | −33.768 | 13.420 | 1.00 | 36.56 |
| ATOM | 13016 | O | LEU | 1939 | 22.114 | −33.980 | 14.101 | 1.00 | 37.71 |
| ATOM | 13017 | N | THR | 1940 | 23.700 | −32.576 | 13.343 | 1.00 | 38.68 |
| ATOM | 13018 | CA | THR | 1940 | 23.192 | −31.417 | 14.070 | 1.00 | 40.34 |
| ATOM | 13019 | CB | THR | 1940 | 24.337 | −30.684 | 14.791 | 1.00 | 40.75 |
| ATOM | 13020 | OG1 | THR | 1940 | 23.398 | −30.420 | 13.864 | 1.00 | 39.49 |
| ATOM | 13021 | OG2 | THR | 1940 | 24.867 | −31.530 | 15.934 | 1.00 | 41.22 |
| ATOM | 13022 | C | THR | 1940 | 22.490 | −30.447 | 13.124 | 1.00 | 41.01 |
| ATOM | 13023 | O | THR | 1940 | 23.139 | −29.714 | 12.379 | 1.00 | 42.38 |
| ATOM | 13024 | N | PRO | 1941 | 21.147 | −30.430 | 13.152 | 1.00 | 42.07 |
| ATOM | 13025 | CD | PRO | 1941 | 20.294 | −31.239 | 14.041 | 1.00 | 43.13 |
| ATOM | 13026 | CA | PRO | 1941 | 20.333 | −29.558 | 12.297 | 1.00 | 42.02 |
| ATOM | 13027 | CB | PRO | 1941 | 18.903 | −29.997 | 12.615 | 1.00 | 42.88 |
| ATOM | 13028 | CG | PRO | 1941 | 18.995 | −30.474 | 14.019 | 1.00 | 44.03 |
| ATOM | 13029 | C | PRO | 1941 | 20.549 | −28.058 | 12.486 | 1.00 | 41.54 |
| ATOM | 13030 | O | PRO | 1941 | 20.198 | −27.265 | 11.611 | 1.00 | 41.77 |
| ATOM | 13031 | N | GLN | 1942 | 21.121 | −27.663 | 13.619 | 1.00 | 40.66 |
| ATOM | 13032 | CA | GLN | 1942 | 21.376 | −26.245 | 13.857 | 1.00 | 40.39 |
| ATOM | 13033 | CB | GLN | 1942 | 21.804 | −26.012 | 15.307 | 1.00 | 39.97 |
| ATOM | 13034 | CG | GLN | 1942 | 20.658 | −25.656 | 16.244 | 1.00 | 38.76 |
| ATOM | 13035 | CD | GLN | 1942 | 21.086 | −25.620 | 17.701 | 1.00 | 38.71 |
| ATOM | 13036 | OE1 | GLN | 1942 | 22.092 | −24.999 | 18.053 | 1.00 | 35.82 |
| ATOM | 13037 | NE2 | GLN | 1942 | 20.316 | −26.284 | 18.559 | 1.00 | 36.70 |
| ATOM | 13038 | C | GLN | 1942 | 22.450 | −25.722 | 12.906 | 1.00 | 40.11 |
| ATOM | 13039 | O | GLN | 1942 | 22.541 | −24.519 | 12.660 | 1.00 | 39.36 |
| ATOM | 13040 | N | SER | 1943 | 23.256 | −26.637 | 12.372 | 1.00 | 40.02 |
| ATOM | 13041 | CA | SER | 1943 | 24.329 | −26.281 | 11.443 | 1.00 | 39.90 |
| ATOM | 13042 | CB | SER | 1943 | 25.637 | −26.959 | 11.862 | 1.00 | 40.15 |
| ATOM | 13043 | OG | SER | 1943 | 25.992 | −26.629 | 13.195 | 1.00 | 42.09 |
| ATOM | 13044 | C | SER | 1943 | 23.978 | −26.703 | 10.017 | 1.00 | 39.67 |
| ATOM | 13045 | O | SER | 1943 | 24.864 | −26.933 | 9.194 | 1.00 | 38.51 |
| ATOM | 13046 | N | VAL | 1944 | 22.682 | −26.805 | 9.734 | 1.00 | 40.21 |
| ATOM | 13047 | CA | VAL | 1944 | 22.209 | −27.205 | 8.411 | 1.00 | 40.13 |
| ATOM | 13048 | CB | VAL | 1944 | 20.660 | −27.155 | 8.335 | 1.00 | 40.32 |
| ATOM | 13049 | CG1 | VAL | 1944 | 20.160 | −25.763 | 8.698 | 1.00 | 40.26 |
| ATOM | 13050 | CG2 | VAL | 1944 | 20.196 | −27.547 | 6.936 | 1.00 | 40.12 |
| ATOM | 13051 | C | VAL | 1944 | 22.794 | −26.352 | 7.284 | 1.00 | 40.09 |
| ATOM | 13052 | O | VAL | 1944 | 23.178 | −26.873 | 6.236 | 1.00 | 40.03 |
| ATOM | 13053 | N | ASN | 1945 | 22.862 | −25.043 | 7.501 | 1.00 | 40.77 |
| ATOM | 13054 | CA | ASN | 1945 | 23.397 | −24.128 | 6.498 | 1.00 | 42.02 |
| ATOM | 13055 | CB | ASN | 1945 | 23.120 | −22.681 | 6.911 | 1.00 | 39.36 |
| ATOM | 13056 | CG | ASN | 1945 | 21.634 | −22.388 | 7.028 | 1.00 | 38.68 |
| ATOM | 13057 | OD1 | ASN | 1945 | 20.905 | −22.428 | 6.035 | 1.00 | 33.53 |
| ATOM | 13058 | ND2 | ASN | 1945 | 21.176 | −22.102 | 8.244 | 1.00 | 34.93 |
| ATOM | 13059 | C | ASN | 1945 | 24.893 | −24.336 | 6.292 | 1.00 | 43.74 |
| ATOM | 13060 | O | ASN | 1945 | 25.413 | −24.102 | 5.200 | 1.00 | 43.69 |
| ATOM | 13061 | N | ILE | 1946 | 25.579 | −24.774 | 7.344 | 1.00 | 46.06 |
| ATOM | 13062 | CA | ILE | 1946 | 27.014 | −25.024 | 7.275 | 1.00 | 48.85 |
| ATOM | 13063 | CB | ILE | 1946 | 27.590 | −25.387 | 8.664 | 1.00 | 48.08 |
| ATOM | 13064 | CG2 | ILE | 1946 | 29.044 | −25.828 | 8.527 | 1.00 | 48.67 |
| ATOM | 13065 | CG1 | ILE | 1946 | 27.476 | −24.188 | 9.609 | 1.00 | 47.10 |
| ATOM | 13066 | CD1 | ILE | 1946 | 28.319 | −22.994 | 9.203 | 1.00 | 45.47 |
| ATOM | 13067 | C | ILE | 1946 | 27.294 | −26.176 | 6.316 | 1.00 | 51.55 |
| ATOM | 13068 | O | ILE | 1946 | 27.949 | −26.001 | 5.287 | 1.00 | 51.88 |
| ATOM | 13069 | N | PHE | 1947 | 26.788 | −27.354 | 6.666 | 1.00 | 54.57 |
| ATOM | 13070 | CA | PHE | 1947 | 26.970 | −28.551 | 5.855 | 1.00 | 57.12 |
| ATOM | 13071 | CB | PHE | 1947 | 26.427 | −29.770 | 6.605 | 1.00 | 59.02 |
| ATOM | 13072 | CG | PHE | 1947 | 27.106 | −30.023 | 7.924 | 1.00 | 61.39 |
| ATOM | 13073 | CD1 | PHE | 1947 | 28.397 | −30.544 | 7.969 | 1.00 | 62.43 |
| ATOM | 13074 | CD2 | PHE | 1947 | 26.461 | −29.726 | 9.121 | 1.00 | 61.93 |
| ATOM | 13075 | CE1 | PHE | 1947 | 29.036 | −30.767 | 9.189 | 1.00 | 62.77 |
| ATOM | 13076 | CE2 | PHE | 1947 | 27.090 | −29.945 | 10.345 | 1.00 | 62.58 |
| ATOM | 13077 | CZ | PHE | 1947 | 28.380 | −30.467 | 10.379 | 1.00 | 62.90 |
| ATOM | 13078 | C | PHE | 1947 | 26.260 | −28.407 | 4.514 | 1.00 | 58.08 |
| ATOM | 13079 | O | PHE | 1947 | 26.884 | −28.505 | 3.455 | 1.00 | 59.24 |
| ATOM | 13080 | N | GLY | 1948 | 24.954 | −28.169 | 4.566 | 1.00 | 58.06 |
| ATOM | 13081 | CA | GLY | 1948 | 24.180 | −28.015 | 3.349 | 1.00 | 58.62 |
| ATOM | 13082 | C | GLY | 1948 | 22.862 | −28.761 | 3.410 | 1.00 | 59.00 |
| ATOM | 13083 | O | GLY | 1948 | 22.084 | −28.740 | 2.458 | 1.00 | 58.82 |
| ATOM | 13084 | N | GLY | 1949 | 22.613 | −29.420 | 4.537 | 1.00 | 59.35 |
| ATOM | 13085 | CA | GLY | 1949 | 21.381 | −30.168 | 4.701 | 1.00 | 60.55 |
| ATOM | 13086 | C | GLY | 1949 | 21.459 | −31.129 | 5.870 | 1.00 | 61.51 |
| ATOM | 13087 | O | GLY | 1949 | 22.026 | −30.801 | 6.914 | 1.00 | 61.37 |
| ATOM | 13088 | N | TYR | 1950 | 20.889 | −32.319 | 5.695 | 1.00 | 62.15 |
| ATOM | 13089 | CA | TYR | 1950 | 20.898 | −33.337 | 6.745 | 1.00 | 62.76 |
| ATOM | 13090 | CB | TYR | 1950 | 19.524 | −33.419 | 7.420 | 1.00 | 63.27 |
| ATOM | 13091 | CG | TYR | 1950 | 18.892 | −32.075 | 7.706 | 1.00 | 63.93 |
| ATOM | 13092 | CD1 | TYR | 1950 | 18.228 | −31.368 | 6.702 | 1.00 | 64.37 |
| ATOM | 13093 | CE1 | TYR | 1950 | 17.653 | −30.124 | 6.955 | 1.00 | 64.90 |
| ATOM | 13094 | CD2 | TYR | 1950 | 18.967 | −31.502 | 8.975 | 1.00 | 64.29 |
| ATOM | 13095 | CE2 | TYR | 1950 | 18.398 | −30.258 | 9.239 | 1.00 | 64.47 |
| ATOM | 13096 | CZ | TYR | 1950 | 17.741 | −29.576 | 8.225 | 1.00 | 65.14 |
| ATOM | 13097 | OH | TYR | 1950 | 17.169 | −28.347 | 8.477 | 1.00 | 65.21 |
| ATOM | 13098 | C | TYR | 1950 | 21.266 | −34.703 | 6.163 | 1.00 | 62.54 |
| ATOM | 13099 | O | TYR | 1950 | 20.515 | −35.272 | 5.371 | 1.00 | 62.37 |
| ATOM | 13100 | N | LYS | 1951 | 22.423 | −35.223 | 6.564 | 1.00 | 62.40 |
| ATOM | 13101 | CA | LYS | 1951 | 22.898 | −36.517 | 6.081 | 1.00 | 62.04 |
| ATOM | 13102 | CB | LYS | 1951 | 23.874 | −36.315 | 4.919 | 1.00 | 62.86 |
| ATOM | 13103 | CG | LYS | 1951 | 23.260 | −35.611 | 3.721 | 1.00 | 64.49 |
| ATOM | 13104 | CD | LYS | 1951 | 24.309 | −35.241 | 2.688 | 1.00 | 65.92 |
| ATOM | 13105 | CE | LYS | 1951 | 23.687 | −34.471 | 1.532 | 1.00 | 66.41 |
| ATOM | 13106 | NZ | LYS | 1951 | 24.709 | −33.991 | 0.560 | 1.00 | 67.31 |
| ATOM | 13107 | C | LYS | 1951 | 23.579 | −37.316 | 7.190 | 1.00 | 60.75 |
| ATOM | 13108 | O | LYS | 1951 | 24.018 | −36.755 | 8.195 | 1.00 | 60.94 |
| ATOM | 13109 | N | VAL | 1952 | 23.659 | −38.630 | 6.999 | 1.00 | 58.89 |
| ATOM | 13110 | CA | VAL | 1952 | 24.284 | −39.511 | 7.979 | 1.00 | 57.24 |
| ATOM | 13111 | CB | VAL | 1952 | 24.287 | −40.975 | 7.491 | 1.00 | 57.00 |
| ATOM | 13112 | CG1 | VAL | 1952 | 24.913 | −41.875 | 8.545 | 1.00 | 57.09 |
| ATOM | 13113 | CG2 | VAL | 1952 | 22.868 | −41.422 | 7.184 | 1.00 | 56.86 |
| ATOM | 13114 | C | VAL | 1952 | 25.722 | −39.079 | 8.243 | 1.00 | 56.27 |
| ATOM | 13115 | O | VAL | 1952 | 26.511 | −38.917 | 7.313 | 1.00 | 55.83 |
| ATOM | 13116 | N | GLN | 1953 | 26.055 | −38.898 | 9.517 | 1.00 | 54.84 |
| ATOM | 13117 | CA | GLN | 1953 | 27.395 | −38.479 | 9.907 | 1.00 | 53.67 |
| ATOM | 13118 | CB | GLN | 1953 | 27.303 | −37.270 | 10.841 | 1.00 | 53.92 |
| ATOM | 13119 | CG | GLN | 1953 | 28.623 | −36.560 | 11.083 | 1.00 | 55.91 |
| ATOM | 13120 | CD | GLN | 1953 | 29.188 | −35.934 | 9.820 | 1.00 | 56.76 |
| ATOM | 13121 | OE1 | GLN | 1953 | 28.532 | −35.114 | 9.174 | 1.00 | 55.89 |
| ATOM | 13122 | NE2 | GLN | 1953 | 30.413 | −36.318 | 9.463 | 1.00 | 56.77 |
| ATOM | 13123 | C | GLN | 1953 | 28.137 | −39.623 | 10.600 | 1.00 | 52.67 |
| ATOM | 13124 | O | GLN | 1953 | 27.517 | −40.550 | 11.116 | 1.00 | 52.18 |
| ATOM | 13125 | N | GLY | 1954 | 29.466 | −39.558 | 10.598 | 1.00 | 51.97 |
| ATOM | 13126 | CA | GLY | 1954 | 30.256 | −40.598 | 11.236 | 1.00 | 51.94 |
| ATOM | 13127 | C | GLY | 1954 | 30.855 | −41.601 | 10.266 | 1.00 | 51.75 |
| ATOM | 13128 | O | GLY | 1954 | 31.612 | −42.488 | 10.664 | 1.00 | 51.15 |
| ATOM | 13129 | N | ARG | 1955 | 30.514 | −41.462 | 8.990 | 1.00 | 51.95 |
| ATOM | 13130 | CA | ARG | 1955 | 31.018 | −42.353 | 7.953 | 1.00 | 52.89 |
| ATOM | 13131 | CB | ARG | 1955 | 30.331 | −42.042 | 6.619 | 1.00 | 54.06 |
| ATOM | 13132 | CG | ARG | 1955 | 28.840 | −42.353 | 6.588 | 1.00 | 56.32 |
| ATOM | 13133 | CD | ARG | 1955 | 28.588 | −43.852 | 6.597 | 1.00 | 58.03 |
| ATOM | 13134 | NE | ARG | 1955 | 27.164 | −44.184 | 6.599 | 1.00 | 59.33 |
| ATOM | 13135 | CZ | ARG | 1955 | 26.313 | −43.872 | 5.625 | 1.00 | 59.73 |
| ATOM | 13136 | NH1 | ARG | 1955 | 26.735 | −43.212 | 4.554 | 1.00 | 59.72 |
| ATOM | 13137 | NH2 | ARG | 1955 | 25.036 | −44.223 | 5.720 | 1.00 | 60.10 |
| ATOM | 13138 | C | ARG | 1955 | 32.529 | −42.205 | 7.797 | 1.00 | 52.91 |
| ATOM | 13139 | O | ARG | 1955 | 33.034 | −41.102 | 7.582 | 1.00 | 52.93 |
| ATOM | 13140 | N | GLY | 1956 | 33.245 | −43.320 | 7.909 | 1.00 | 52.81 |
| ATOM | 13141 | CA | GLY | 1956 | 34.692 | −43.290 | 7.771 | 1.00 | 52.45 |
| ATOM | 13142 | C | GLY | 1956 | 35.420 | −43.286 | 9.101 | 1.00 | 51.95 |
| ATOM | 13143 | O | GLY | 1956 | 34.882 | −42.834 | 10.110 | 1.00 | 52.28 |
| ATOM | 13144 | N | ASP | 1957 | 36.650 | −43.787 | 9.101 | 1.00 | 51.06 |
| ATOM | 13145 | CA | ASP | 1957 | 37.450 | −43.841 | 10.319 | 1.00 | 50.18 |
| ATOM | 13146 | CB | ASP | 1957 | 38.777 | −44.553 | 10.050 | 1.00 | 52.74 |
| ATOM | 13147 | CG | ASP | 1957 | 38.584 | −45.974 | 9.572 | 1.00 | 55.34 |
| ATOM | 13148 | OD1 | ASP | 1957 | 37.909 | −46.752 | 10.283 | 1.00 | 56.35 |
| ATOM | 13149 | OD2 | ASP | 1957 | 39.109 | −46.315 | 8.489 | 1.00 | 57.00 |
| ATOM | 13150 | C | ASP | 1957 | 37.727 | −42.446 | 10.867 | 1.00 | 47.95 |
| ATOM | 13151 | O | ASP | 1957 | 37.620 | −42.209 | 12.069 | 1.00 | 47.47 |
| ATOM | 13152 | N | GLU | 1958 | 38.087 | −41.529 | 9.977 | 1.00 | 45.13 |
| ATOM | 13153 | CA | GLU | 1958 | 38.387 | −40.160 | 10.368 | 1.00 | 43.01 |
| ATOM | 13154 | CB | GLU | 1958 | 38.677 | −39.318 | 9.127 | 1.00 | 45.74 |
| ATOM | 13155 | CG | GLU | 1958 | 39.190 | −37.924 | 9.429 | 1.00 | 48.92 |
| ATOM | 13156 | CD | GLU | 1958 | 39.353 | −37.092 | 8.174 | 1.00 | 51.94 |
| ATOM | 13157 | OE1 | GLU | 1958 | 39.989 | −37.585 | 7.215 | 1.00 | 52.78 |
| ATOM | 13158 | OE2 | GLU | 1958 | 38.850 | −35.947 | 8.148 | 1.00 | 53.38 |
| ATOM | 13159 | C | GLU | 1958 | 37.231 | −39.540 | 11.146 | 1.00 | 40.27 |
| ATOM | 13160 | O | GLU | 1958 | 37.380 | −39.175 | 12.313 | 1.00 | 37.28 |
| ATOM | 13161 | N | ALA | 1959 | 36.079 | −39.424 | 10.493 | 1.00 | 36.37 |
| ATOM | 13162 | CA | ALA | 1959 | 34.900 | −38.846 | 11.129 | 1.00 | 34.01 |
| ATOM | 13163 | CB | ALA | 1959 | 33.754 | −38.760 | 10.130 | 1.00 | 33.11 |
| ATOM | 13164 | C | ALA | 1959 | 34.484 | −39.683 | 12.333 | 1.00 | 31.93 |
| ATOM | 13165 | O | ALA | 1959 | 34.046 | −39.149 | 13.353 | 1.00 | 31.65 |
| ATOM | 13166 | N | GLY | 1960 | 34.625 | −40.997 | 12.203 | 1.00 | 30.55 |
| ATOM | 13167 | CA | GLY | 1960 | 34.262 | −41.895 | 13.283 | 1.00 | 29.48 |
| ATOM | 13168 | C | GLY | 1960 | 35.089 | −41.676 | 14.535 | 1.00 | 28.48 |
| ATOM | 13169 | O | GLY | 1960 | 34.553 | −41.644 | 15.644 | 1.00 | 27.01 |
| ATOM | 13170 | N | ASP | 1961 | 36.397 | −41.520 | 14.365 | 1.00 | 27.04 |
| ATOM | 13171 | CA | ASP | 1961 | 37.284 | −41.310 | 15.502 | 1.00 | 27.15 |
| ATOM | 13172 | CB | ASP | 1961 | 38.752 | −41.439 | 15.076 | 1.00 | 29.17 |
| ATOM | 13173 | CG | ASP | 1961 | 39.120 | −42.849 | 14.653 | 1.00 | 31.98 |
| ATOM | 13174 | OD1 | ASP | 1961 | 38.398 | −43.797 | 15.040 | 1.00 | 30.19 |
| ATOM | 13175 | OD2 | ASP | 1961 | 40.143 | −43.009 | 13.944 | 1.00 | 33.17 |
| ATOM | 13176 | C | ASP | 1961 | 37.053 | −39.947 | 16.135 | 1.00 | 25.99 |
| ATOM | 13177 | O | ASP | 1961 | 37.114 | −39.803 | 17.358 | 1.00 | 25.23 |
| ATOM | 13178 | N | GLN | 1962 | 36.791 | −38.945 | 15.301 | 1.00 | 24.65 |
| ATOM | 13179 | CA | GLN | 1962 | 36.549 | −37.598 | 15.803 | 1.00 | 24.43 |
| ATOM | 13180 | CB | GLN | 1962 | 36.404 | −36.613 | 14.644 | 1.00 | 25.79 |
| ATOM | 13181 | CG | GLN | 1962 | 36.228 | −35.174 | 15.095 | 1.00 | 28.89 |
| ATOM | 13182 | CD | GLN | 1962 | 37.400 | −34.677 | 15.912 | 1.00 | 31.20 |
| ATOM | 13183 | OE1 | GLN | 1962 | 38.524 | −34.600 | 15.419 | 1.00 | 31.16 |
| ATOM | 13184 | NE2 | GLN | 1962 | 37.144 | −34.339 | 17.172 | 1.00 | 32.91 |
| ATOM | 13185 | C | GLN | 1962 | 35.295 | −37.556 | 16.678 | 1.00 | 22.32 |
| ATOM | 13186 | O | GLN | 1962 | 35.267 | −36.860 | 17.691 | 1.00 | 20.87 |
| ATOM | 13187 | N | LEU | 1963 | 34.258 | −38.294 | 16.285 | 1.00 | 21.33 |
| ATOM | 13188 | CA | LEU | 1963 | 33.032 | −38.323 | 17.068 | 1.00 | 21.71 |
| ATOM | 13189 | CB | LEU | 1963 | 31.900 | −39.012 | 16.293 | 1.00 | 22.46 |
| ATOM | 13190 | CG | LEU | 1963 | 31.338 | −38.237 | 15.092 | 1.00 | 28.54 |
| ATOM | 13191 | CD1 | LEU | 1963 | 30.208 | −39.027 | 14.450 | 1.00 | 28.46 |
| ATOM | 13192 | CD2 | LEU | 1963 | 30.822 | −36.868 | 15.550 | 1.00 | 27.56 |
| ATOM | 13193 | C | LEU | 1963 | 33.274 | −39.044 | 18.392 | 1.00 | 20.51 |
| ATOM | 13194 | O | LEU | 1963 | 32.745 | −38.641 | 19.425 | 1.00 | 18.17 |
| ATOM | 13195 | N | LEU | 1964 | 34.074 | −40.109 | 18.359 | 1.00 | 19.90 |
| ATOM | 13196 | CA | LEU | 1964 | 34.387 | −40.860 | 19.573 | 1.00 | 18.82 |
| ATOM | 13197 | CB | LEU | 1964 | 35.293 | −42.060 | 19.258 | 1.00 | 19.20 |
| ATOM | 13198 | CG | LEU | 1964 | 35.150 | −43.326 | 20.114 | 1.00 | 22.73 |
| ATOM | 13199 | CD1 | LEU | 1964 | 36.408 | −44.170 | 19.984 | 1.00 | 21.26 |
| ATOM | 13200 | CD2 | LEU | 1964 | 34.908 | −42.985 | 21.568 | 1.00 | 21.92 |
| ATOM | 13201 | C | LEU | 1964 | 35.125 | −39.916 | 20.513 | 1.00 | 18.01 |
| ATOM | 13202 | O | LEU | 1964 | 34.827 | −39.843 | 21.704 | 1.00 | 16.75 |
| ATOM | 13203 | N | SER | 1965 | 36.097 | −39.197 | 19.958 | 1.00 | 19.21 |
| ATOM | 13204 | CA | SER | 1965 | 36.891 | −38.251 | 20.726 | 1.00 | 18.88 |
| ATOM | 13205 | CB | SER | 1965 | 37.939 | −37.586 | 19.834 | 1.00 | 19.39 |
| ATOM | 13206 | OG | SER | 1965 | 38.736 | −36.691 | 20.592 | 1.00 | 21.10 |
| ATOM | 13207 | C | SER | 1965 | 36.017 | −37.179 | 21.354 | 1.00 | 17.02 |
| ATOM | 13208 | O | SER | 1965 | 36.122 | −36.915 | 22.550 | 1.00 | 15.35 |
| ATOM | 13209 | N | ASP | 1966 | 35.156 | −36.558 | 20.549 | 1.00 | 16.60 |
| ATOM | 13210 | CA | ASP | 1966 | 34.278 | −35.509 | 21.071 | 1.00 | 17.92 |
| ATOM | 13211 | CB | ASP | 1966 | 33.404 | −34.910 | 19.964 | 1.00 | 18.63 |
| ATOM | 13212 | CG | ASP | 1966 | 34.197 | −34.078 | 18.974 | 1.00 | 19.16 |
| ATOM | 13213 | OD1 | ASP | 1966 | 35.254 | −33.536 | 19.352 | 1.00 | 24.28 |
| ATOM | 13214 | OD2 | ASP | 1966 | 33.745 | −33.954 | 17.819 | 1.00 | 20.44 |
| ATOM | 13215 | C | ASP | 1966 | 33.378 | −36.061 | 22.170 | 1.00 | 15.95 |
| ATOM | 13216 | O | ASP | 1966 | 33.162 | −35.409 | 23.194 | 1.00 | 15.58 |
| ATOM | 13217 | N | ALA | 1967 | 32.852 | −37.263 | 21.955 | 1.00 | 15.46 |
| ATOM | 13218 | CA | ALA | 1967 | 31.980 | −37.889 | 22.946 | 1.00 | 13.99 |
| ATOM | 13219 | CB | ALA | 1967 | 31.536 | −39.264 | 22.454 | 1.00 | 13.81 |
| ATOM | 13220 | C | ALA | 1967 | 32.703 | −38.012 | 24.280 | 1.00 | 14.10 |
| ATOM | 13221 | O | ALA | 1967 | 32.157 | −37.674 | 25.333 | 1.00 | 14.44 |
| ATOM | 13222 | N | LEU | 1968 | 33.944 | −38.490 | 24.240 | 1.00 | 13.09 |
| ATOM | 13223 | CA | LEU | 1968 | 34.724 | −38.650 | 25.463 | 1.00 | 13.85 |
| ATOM | 13224 | CB | LEU | 1968 | 36.010 | −39.434 | 25.164 | 1.00 | 14.52 |
| ATOM | 13225 | CG | LEU | 1968 | 35.805 | −40.946 | 24.954 | 1.00 | 14.84 |
| ATOM | 13226 | CD1 | LEU | 1968 | 37.006 | −41.547 | 24.234 | 1.00 | 16.18 |
| ATOM | 13227 | CD2 | LEU | 1968 | 35.599 | −41.618 | 26.306 | 1.00 | 17.51 |
| ATOM | 13228 | C | LEU | 1968 | 35.062 | −37.293 | 26.088 | 1.00 | 13.86 |
| ATOM | 13229 | O | LEU | 1968 | 35.056 | −37.157 | 27.309 | 1.00 | 14.68 |
| ATOM | 13230 | N | ALA | 1969 | 35.342 | −36.290 | 25.257 | 1.00 | 14.00 |
| ATOM | 13231 | CA | ALA | 1969 | 35.672 | −34.957 | 25.769 | 1.00 | 14.01 |
| ATOM | 13232 | CB | ALA | 1969 | 36.150 | −34.057 | 24.634 | 1.00 | 14.15 |
| ATOM | 13233 | C | ALA | 1969 | 34.468 | −34.325 | 26.462 | 1.00 | 13.52 |
| ATOM | 13234 | O | ALA | 1969 | 34.609 | −33.634 | 27.476 | 1.00 | 12.39 |
| ATOM | 13235 | N | LEU | 1970 | 33.283 | −34.552 | 25.909 | 1.00 | 15.34 |
| ATOM | 13236 | CA | LEU | 1970 | 32.060 | −34.005 | 26.502 | 1.00 | 14.25 |
| ATOM | 13237 | CB | LEU | 1970 | 30.869 | −34.220 | 25.563 | 1.00 | 15.21 |
| ATOM | 13238 | CG | LEU | 1970 | 30.929 | −33.394 | 24.272 | 1.00 | 16.66 |
| ATOM | 13239 | CD1 | LEU | 1970 | 29.795 | −33.811 | 23.329 | 1.00 | 17.51 |
| ATOM | 13240 | CD2 | LEU | 1970 | 30.815 | −31.913 | 24.610 | 1.00 | 16.43 |
| ATOM | 13241 | C | LEU | 1970 | 31.806 | −34.661 | 27.848 | 1.00 | 13.96 |
| ATOM | 13242 | O | LEU | 1970 | 31.474 | −33.982 | 28.824 | 1.00 | 12.41 |
| ATOM | 13243 | N | GLU | 1971 | 31.981 | −35.980 | 27.907 | 1.00 | 12.89 |
| ATOM | 13244 | CA | GLU | 1971 | 31.787 | −36.705 | 29.162 | 1.00 | 14.56 |
| ATOM | 13245 | CB | GLU | 1971 | 31.951 | −38.224 | 28.945 | 1.00 | 16.29 |
| ATOM | 13246 | CG | GLU | 1971 | 31.840 | −39.074 | 30.226 | 1.00 | 15.90 |
| ATOM | 13247 | CD | GLU | 1971 | 32.114 | −40.557 | 29.981 | 1.00 | 15.83 |
| ATOM | 13248 | OE1 | GLU | 1971 | 33.169 | −40.888 | 29.415 | 1.00 | 18.88 |
| ATOM | 13249 | OE2 | GLU | 1971 | 31.279 | −41.392 | 30.358 | 1.00 | 19.45 |
| ATOM | 13250 | C | GLU | 1971 | 32.805 | −36.207 | 30.195 | 1.00 | 15.16 |
| ATOM | 13251 | O | GLU | 1971 | 32.465 | −35.965 | 31.353 | 1.00 | 13.13 |
| ATOM | 13252 | N | ALA | 1972 | 34.055 | −36.039 | 29.774 | 1.00 | 14.67 |
| ATOM | 13253 | CA | ALA | 1972 | 35.087 | −35.589 | 30.695 | 1.00 | 15.71 |
| ATOM | 13254 | CB | ALA | 1972 | 36.465 | −35.643 | 30.015 | 1.00 | 15.78 |
| ATOM | 13255 | C | ALA | 1972 | 34.802 | −34.181 | 31.192 | 1.00 | 15.67 |
| ATOM | 13256 | O | ALA | 1972 | 35.151 | −33.832 | 32.323 | 1.00 | 17.06 |
| ATOM | 13257 | N | ALA | 1973 | 34.167 | −33.377 | 30.342 | 1.00 | 16.83 |
| ATOM | 13258 | CA | ALA | 1973 | 33.826 | −31.993 | 30.673 | 1.00 | 16.03 |
| ATOM | 13259 | CB | ALA | 1973 | 33.434 | −31.243 | 29.399 | 1.00 | 17.16 |
| ATOM | 13260 | C | ALA | 1973 | 32.690 | −31.924 | 31.694 | 1.00 | 16.25 |
| ATOM | 13261 | O | ALA | 1973 | 32.499 | −30.900 | 32.354 | 1.00 | 16.68 |
| ATOM | 13262 | N | GLY | 1974 | 31.939 | −33.013 | 31.824 | 1.00 | 15.27 |
| ATOM | 13263 | CA | GLY | 1974 | 30.857 | −33.037 | 32.790 | 1.00 | 15.24 |
| ATOM | 13264 | C | GLY | 1974 | 29.473 | −33.342 | 32.251 | 1.00 | 13.22 |
| ATOM | 13265 | O | GLY | 1974 | 28.498 | −33.327 | 33.008 | 1.00 | 13.69 |
| ATOM | 13266 | N | ALA | 1975 | 29.357 | −33.606 | 30.954 | 1.00 | 13.87 |
| ATOM | 13267 | CA | ALA | 1975 | 28.038 | −33.927 | 30.396 | 1.00 | 12.92 |
| ATOM | 13268 | CB | ALA | 1975 | 28.126 | −34.066 | 28.888 | 1.00 | 12.07 |
| ATOM | 13269 | C | ALA | 1975 | 27.581 | −35.243 | 31.031 | 1.00 | 12.54 |
| ATOM | 13270 | O | ALA | 1975 | 28.364 | −36.188 | 31.104 | 1.00 | 11.18 |
| ATOM | 13271 | N | GLN | 1976 | 26.330 | −35.304 | 31.492 | 1.00 | 11.89 |
| ATOM | 13272 | CA | GLN | 1976 | 25.803 | −36.514 | 32.132 | 1.00 | 11.40 |
| ATOM | 13273 | CB | GLN | 1976 | 25.009 | −36.150 | 33.387 | 1.00 | 12.41 |
| ATOM | 13274 | CG | GLN | 1976 | 25.890 | −35.701 | 34.561 | 1.00 | 15.82 |
| ATOM | 13275 | CD | GLN | 1976 | 25.091 | −35.135 | 35.720 | 1.00 | 16.62 |
| ATOM | 13276 | OE1 | GLN | 1976 | 24.747 | −35.845 | 36.671 | 1.00 | 21.24 |
| ATOM | 13277 | NE2 | GLN | 1976 | 24.784 | −33.847 | 35.641 | 1.00 | 14.90 |
| ATOM | 13278 | C | GLN | 1976 | 24.931 | −37.348 | 31.196 | 1.00 | 11.43 |
| ATOM | 13279 | O | GLN | 1976 | 24.407 | −38.397 | 31.581 | 1.00 | 11.61 |
| ATOM | 13280 | N | LEU | 1977 | 24.780 | −36.862 | 29.970 | 1.00 | 10.84 |
| ATOM | 13281 | CA | LEU | 1977 | 23.986 | −37.533 | 28.957 | 1.00 | 12.72 |
| ATOM | 13282 | CB | LEU | 1977 | 22.510 | −37.145 | 29.089 | 1.00 | 15.93 |
| ATOM | 13283 | CG | LEU | 1977 | 21.602 | −38.134 | 29.806 | 1.00 | 21.02 |
| ATOM | 13284 | CD1 | LEU | 1977 | 20.255 | −37.461 | 30.072 | 1.00 | 21.00 |
| ATOM | 13285 | CD2 | LEU | 1977 | 21.419 | −39.379 | 28.955 | 1.00 | 23.10 |
| ATOM | 13286 | C | LEU | 1977 | 24.476 | −37.130 | 27.588 | 1.00 | 11.33 |
| ATOM | 13287 | O | LEU | 1977 | 25.024 | −36.050 | 27.414 | 1.00 | 12.24 |
| ATOM | 13288 | N | LEU | 1978 | 24.253 | −38.004 | 26.614 | 1.00 | 11.93 |
| ATOM | 13289 | CA | LEU | 1978 | 24.673 | −37.739 | 25.247 | 1.00 | 12.10 |
| ATOM | 13290 | CB | LEU | 1978 | 26.066 | −38.341 | 25.001 | 1.00 | 13.29 |
| ATOM | 13291 | CG | LEU | 1978 | 26.488 | −38.431 | 23.533 | 1.00 | 16.13 |
| ATOM | 13292 | CD1 | LEU | 1978 | 26.589 | −37.031 | 22.943 | 1.00 | 16.64 |
| ATOM | 13293 | CD2 | LEU | 1978 | 27.819 | −39.159 | 23.422 | 1.00 | 12.84 |
| ATOM | 13294 | C | LEU | 1978 | 23.688 | −38.311 | 24.238 | 1.00 | 12.27 |
| ATOM | 13295 | O | LEU | 1978 | 23.223 | −39.445 | 24.374 | 1.00 | 13.09 |
| ATOM | 13296 | N | VAL | 1979 | 23.356 | −37.503 | 23.239 | 1.00 | 11.61 |
| ATOM | 13297 | CA | VAL | 1979 | 22.456 | −37.911 | 22.159 | 1.00 | 10.55 |
| ATOM | 13298 | CB | VAL | 1979 | 21.370 | −36.815 | 21.854 | 1.00 | 10.14 |
| ATOM | 13299 | CG1 | VAL | 1979 | 20.708 | −37.068 | 20.492 | 1.00 | 10.01 |
| ATOM | 13300 | CG2 | VAL | 1979 | 20.321 | −36.812 | 22.935 | 1.00 | 9.21 |
| ATOM | 13301 | C | VAL | 1979 | 23.333 | −38.099 | 20.926 | 1.00 | 12.11 |
| ATOM | 13302 | O | VAL | 1979 | 24.206 | −37.274 | 20.637 | 1.00 | 13.15 |
| ATOM | 13303 | N | LEU | 1980 | 23.119 | −39.216 | 20.238 | 1.00 | 12.53 |
| ATOM | 13304 | CA | LEU | 1980 | 23.829 | −39.547 | 19.009 | 1.00 | 14.61 |
| ATOM | 13305 | CB | LEU | 1980 | 24.510 | −40.913 | 19.103 | 1.00 | 16.21 |
| ATOM | 13306 | CG | LEU | 1980 | 25.859 | −41.049 | 19.799 | 1.00 | 21.32 |
| ATOM | 13307 | CD1 | LEU | 1980 | 26.338 | −42.498 | 19.648 | 1.00 | 17.71 |
| ATOM | 13308 | CD2 | LEU | 1980 | 26.877 | −40.094 | 19.170 | 1.00 | 18.47 |
| ATOM | 13309 | C | LEU | 1980 | 22.747 | −39.616 | 17.946 | 1.00 | 15.13 |
| ATOM | 13310 | O | LEU | 1980 | 21.812 | −40.411 | 18.060 | 1.00 | 12.67 |
| ATOM | 13311 | N | GLU | 1981 | 22.880 | −38.786 | 16.916 | 1.00 | 14.35 |
| ATOM | 13312 | CA | GLU | 1981 | 21.897 | −38.726 | 15.851 | 1.00 | 17.03 |
| ATOM | 13313 | CB | GLU | 1981 | 21.298 | −37.314 | 15.799 | 1.00 | 15.90 |
| ATOM | 13314 | CG | GLU | 1981 | 20.445 | −37.013 | 14.584 | 1.00 | 20.75 |
| ATOM | 13315 | CD | GLU | 1981 | 19.621 | −35.740 | 14.747 | 1.00 | 23.90 |
| ATOM | 13316 | OE1 | GLU | 1981 | 20.106 | −34.784 | 15.389 | 1.00 | 25.09 |
| ATOM | 13317 | OE2 | GLU | 1981 | 18.492 | −35.692 | 14.221 | 1.00 | 26.95 |
| ATOM | 13318 | C | GLU | 1981 | 22.424 | −39.120 | 14.479 | 1.00 | 17.13 |
| ATOM | 13319 | O | GLU | 1981 | 23.429 | −38.590 | 14.007 | 1.00 | 18.47 |
| ATOM | 13320 | N | CYS | 1982 | 21.733 | −40.065 | 13.849 | 1.00 | 17.94 |
| ATOM | 13321 | CA | CYS | 1982 | 22.080 | −40.532 | 12.513 | 1.00 | 19.05 |
| ATOM | 13322 | CB | CYS | 1982 | 21.599 | −39.505 | 11.489 | 1.00 | 18.86 |
| ATOM | 13323 | SG | CYS | 1982 | 19.782 | −39.350 | 11.492 | 1.00 | 25.27 |
| ATOM | 13324 | C | CYS | 1982 | 23.554 | −40.847 | 12.313 | 1.00 | 20.26 |
| ATOM | 13325 | O | CYS | 1982 | 24.276 | −40.134 | 11.616 | 1.00 | 21.64 |
| ATOM | 13326 | N | VAL | 1983 | 23.980 | −41.947 | 12.920 | 1.00 | 21.57 |
| ATOM | 13327 | CA | VAL | 1983 | 25.359 | −42.399 | 12.844 | 1.00 | 22.20 |
| ATOM | 13328 | CB | VAL | 1983 | 26.087 | −42.075 | 14.172 | 1.00 | 23.68 |
| ATOM | 13329 | CG1 | VAL | 1983 | 25.549 | −42.952 | 15.287 | 1.00 | 24.53 |
| ATOM | 13330 | CG2 | VAL | 1983 | 27.573 | −42.253 | 14.021 | 1.00 | 26.35 |
| ATOM | 13331 | C | VAL | 1983 | 25.351 | −43.913 | 12.614 | 1.00 | 21.11 |
| ATOM | 13332 | O | VAL | 1983 | 24.415 | −44.605 | 13.011 | 1.00 | 19.74 |
| ATOM | 13333 | N | PRO | 1984 | 26.382 | −44.450 | 11.945 | 1.00 | 21.78 |
| ATOM | 13334 | CD | PRO | 1984 | 27.550 | −43.820 | 11.312 | 1.00 | 23.46 |
| ATOM | 13335 | CA | PRO | 1984 | 26.390 | −45.898 | 11.724 | 1.00 | 20.82 |
| ATOM | 13336 | CB | PRO | 1984 | 27.718 | −46.130 | 10.992 | 1.00 | 23.68 |
| ATOM | 13337 | CG | PRO | 1984 | 28.555 | −44.935 | 11.367 | 1.00 | 24.78 |
| ATOM | 13338 | C | PRO | 1984 | 26.296 | −46.663 | 13.043 | 1.00 | 18.82 |
| ATOM | 13339 | O | PRO | 1984 | 26.897 | −46.268 | 14.040 | 1.00 | 18.80 |
| ATOM | 13340 | N | VAL | 1985 | 25.536 | −47.753 | 13.038 | 1.00 | 18.79 |
| ATOM | 13341 | CA | VAL | 1985 | 25.336 | −48.578 | 14.225 | 1.00 | 19.02 |
| ATOM | 13342 | CB | VAL | 1985 | 24.558 | −49.873 | 13.874 | 1.00 | 20.60 |
| ATOM | 13343 | CG1 | VAL | 1985 | 24.312 | −50.691 | 15.122 | 1.00 | 20.17 |
| ATOM | 13344 | CG2 | VAL | 1985 | 23.236 | −49.524 | 13.214 | 1.00 | 17.11 |
| ATOM | 13345 | C | VAL | 1985 | 26.634 | −48.980 | 14.909 | 1.00 | 20.44 |
| ATOM | 13346 | O | VAL | 1985 | 26.732 | −48.931 | 16.136 | 1.00 | 19.37 |
| ATOM | 13347 | N | GLU | 1986 | 27.630 | −49.383 | 14.122 | 1.00 | 20.52 |
| ATOM | 13348 | CA | GLU | 1986 | 28.907 | −49.800 | 14.692 | 1.00 | 21.16 |
| ATOM | 13349 | CB | GLU | 1986 | 29.858 | −50.322 | 13.605 | 1.00 | 23.66 |
| ATOM | 13350 | CG | GLU | 1986 | 29.427 | −50.073 | 12.168 | 1.00 | 30.18 |
| ATOM | 13351 | CD | GLU | 1986 | 28.118 | −50.765 | 11.822 | 1.00 | 31.01 |
| ATOM | 13352 | OE1 | GLU | 1986 | 27.968 | −51.957 | 12.162 | 1.00 | 34.03 |
| ATOM | 13353 | OE2 | GLU | 1986 | 27.251 | −50.116 | 11.207 | 1.00 | 32.54 |
| ATOM | 13354 | C | GLU | 1986 | 29.578 | −48.667 | 15.458 | 1.00 | 19.79 |
| ATOM | 13355 | O | GLU | 1986 | 30.215 | −48.904 | 16.486 | 1.00 | 20.21 |
| ATOM | 13356 | N | LEU | 1987 | 29.435 | −47.440 | 14.959 | 1.00 | 19.82 |
| ATOM | 13357 | CA | LEU | 1987 | 30.032 | −46.288 | 15.629 | 1.00 | 19.57 |
| ATOM | 13358 | CB | LEU | 1987 | 29.973 | −45.039 | 14.743 | 1.00 | 21.80 |
| ATOM | 13359 | CG | LEU | 1987 | 31.137 | −44.044 | 14.841 | 1.00 | 24.62 |
| ATOM | 13360 | CD1 | LEU | 1987 | 30.711 | −42.719 | 14.230 | 1.00 | 24.77 |
| ATOM | 13361 | CD2 | LEU | 1987 | 31.567 | −43.849 | 16.275 | 1.00 | 27.37 |
| ATOM | 13362 | C | LEU | 1987 | 29.278 | −46.015 | 16.930 | 1.00 | 19.16 |
| ATOM | 13363 | O | LEU | 1967 | 29.883 | −45.704 | 17.957 | 1.00 | 19.07 |
| ATOM | 13364 | N | ALA | 1988 | 27.954 | −46.129 | 16.879 | 1.00 | 17.69 |
| ATOM | 13365 | CA | ALA | 1988 | 27.123 | −45.908 | 18.056 | 1.00 | 16.77 |
| ATOM | 13366 | CB | ALA | 1988 | 25.645 | −46.063 | 17.695 | 1.00 | 18.29 |
| ATOM | 13367 | C | ALA | 1988 | 27.510 | −46.919 | 19.131 | 1.00 | 17.28 |
| ATOM | 13368 | O | ALA | 1988 | 27.506 | −46.608 | 20.329 | 1.00 | 15.24 |
| ATOM | 13369 | N | LYS | 1989 | 27.846 | −48.130 | 18.692 | 1.00 | 18.47 |
| ATOM | 13370 | CA | LYS | 1989 | 28.263 | −49.203 | 19.598 | 1.00 | 20.98 |
| ATOM | 13371 | CB | LYS | 1989 | 28.561 | −50.493 | 18.821 | 1.00 | 25.15 |
| ATOM | 13372 | CG | LYS | 1989 | 27.483 | −50.977 | 17.875 | 1.00 | 30.44 |
| ATOM | 13373 | CD | LYS | 1989 | 27.979 | −52.169 | 17.038 | 1.00 | 33.35 |
| ATOM | 13374 | CE | LYS | 1989 | 26.964 | −52.561 | 15.968 | 1.00 | 34.91 |
| ATOM | 13375 | NZ | LYS | 1989 | 27.496 | −53.556 | 15.000 | 1.00 | 37.47 |
| ATOM | 13376 | C | LYS | 1989 | 29.551 | −48.784 | 20.300 | 1.00 | 19.44 |
| ATOM | 13377 | O | LYS | 1989 | 29.656 | −48.853 | 21.523 | 1.00 | 20.34 |
| ATOM | 13378 | N | ARG | 1990 | 30.530 | −48.361 | 19.503 | 1.00 | 18.82 |
| ATOM | 13379 | CA | ARG | 1990 | 31.833 | −47.951 | 20.017 | 1.00 | 18.71 |
| ATOM | 13380 | CB | ARG | 1990 | 32.735 | −47.459 | 18.872 | 1.00 | 23.33 |
| ATOM | 13381 | CG | ARG | 1990 | 34.237 | −47.609 | 19.159 | 1.00 | 28.11 |
| ATOM | 13382 | CD | ARG | 1990 | 35.120 | −46.751 | 18.249 | 1.00 | 31.00 |
| ATOM | 13383 | NE | ARG | 1990 | 34.739 | −46.823 | 16.841 | 1.00 | 33.95 |
| ATOM | 13384 | CZ | ARG | 1990 | 35.335 | −46.132 | 15.871 | 1.00 | 34.97 |
| ATOM | 13385 | NH1 | ARG | 1990 | 36.345 | −45.320 | 16.155 | 1.00 | 34.80 |
| ATOM | 13386 | NH2 | ARG | 1990 | 34.910 | −46.239 | 14.619 | 1.00 | 35.67 |
| ATOM | 13387 | C | ARG | 1990 | 31.691 | −46.848 | 21.058 | 1.00 | 17.38 |
| ATOM | 13388 | O | ARG | 1990 | 32.299 | −46.901 | 22.125 | 1.00 | 16.41 |
| ATOM | 13389 | N | ILE | 1991 | 30.878 | −45.847 | 20.744 | 1.00 | 15.72 |
| ATOM | 13390 | CA | ILE | 1991 | 30.660 | −44.718 | 21.647 | 1.00 | 14.45 |
| ATOM | 13391 | CB | ILE | 1991 | 29.908 | −43.586 | 20.897 | 1.00 | 13.43 |
| ATOM | 13392 | CG2 | ILE | 1991 | 29.506 | −42.468 | 21.887 | 1.00 | 15.10 |
| ATOM | 13393 | CG1 | ILE | 1991 | 30.797 | −43.074 | 19.767 | 1.00 | 13.76 |
| ATOM | 13394 | CD1 | ILE | 1991 | 30.233 | −41.892 | 18.986 | 1.00 | 15.99 |
| ATOM | 13395 | C | ILE | 1991 | 29.916 | −45.087 | 22.933 | 1.00 | 13.45 |
| ATOM | 13396 | O | ILE | 1991 | 30.302 | −44.674 | 24.029 | 1.00 | 14.39 |
| ATOM | 13397 | N | THR | 1992 | 28.856 | −45.877 | 22.799 | 1.00 | 13.96 |
| ATOM | 13398 | CA | THR | 1992 | 28.083 | −46.288 | 23.964 | 1.00 | 13.15 |
| ATOM | 13399 | CB | THR | 1992 | 26.814 | −47.044 | 23.547 | 1.00 | 13.12 |
| ATOM | 13400 | OG1 | THR | 1992 | 26.016 | −46.196 | 22.708 | 1.00 | 13.87 |
| ATOM | 13401 | CG2 | THR | 1992 | 25.991 | −47.432 | 24.779 | 1.00 | 12.06 |
| ATOM | 13402 | C | THR | 1992 | 28.897 | −47.153 | 24.922 | 1.00 | 16.80 |
| ATOM | 13403 | O | THR | 1992 | 28.761 | −47.043 | 26.142 | 1.00 | 15.55 |
| ATOM | 13404 | N | GLU | 1993 | 29.755 | −48.007 | 24.375 | 1.00 | 18.05 |
| ATOM | 13405 | CA | GLU | 1993 | 30.576 | −48.869 | 25.219 | 1.00 | 19.12 |
| ATOM | 13406 | CB | GLU | 1993 | 31.088 | −50.069 | 24.413 | 1.00 | 21.19 |
| ATOM | 13407 | CG | GLU | 1993 | 29.992 | −50.833 | 23.681 | 1.00 | 26.97 |
| ATOM | 13408 | CD | GLU | 1993 | 30.495 | −52.105 | 23.018 | 1.00 | 29.74 |
| ATOM | 13409 | OE1 | GLU | 1993 | 31.684 | −52.156 | 22.646 | 1.00 | 32.85 |
| ATOM | 13410 | OE2 | GLU | 1993 | 29.695 | −53.050 | 22.856 | 1.00 | 31.62 |
| ATOM | 13411 | C | GLU | 1993 | 31.761 | −48.077 | 25.783 | 1.00 | 19.25 |
| ATOM | 13412 | O | GLU | 1993 | 32.323 | −48.440 | 26.814 | 1.00 | 20.28 |
| ATOM | 13413 | N | ALA | 1994 | 32.130 | −46.989 | 25.115 | 1.00 | 17.41 |
| ATOM | 13414 | CA | ALA | 1994 | 33.252 | −46.178 | 25.564 | 1.00 | 18.10 |
| ATOM | 13415 | CB | ALA | 1994 | 33.820 | −45.383 | 24.398 | 1.00 | 18.09 |
| ATOM | 13416 | C | ALA | 1994 | 32.888 | −45.234 | 26.703 | 1.00 | 18.12 |
| ATOM | 13417 | O | ALA | 1994 | 33.716 | −44.928 | 27.565 | 1.00 | 19.20 |
| ATOM | 13418 | N | LEU | 1995 | 31.645 | −44.770 | 26.708 | 1.00 | 17.31 |
| ATOM | 13419 | CA | LEU | 1995 | 31.192 | −43.841 | 27.741 | 1.00 | 17.19 |
| ATOM | 13420 | CB | LEU | 1995 | 30.222 | −42.821 | 27.145 | 1.00 | 17.68 |
| ATOM | 13421 | CG | LEU | 1995 | 30.754 | −41.776 | 26.173 | 1.00 | 18.73 |
| ATOM | 13422 | CD1 | LEU | 1995 | 29.641 | −40.764 | 25.924 | 1.00 | 12.73 |
| ATOM | 13423 | CD2 | LEU | 1995 | 31.976 | −41.083 | 26.718 | 1.00 | 24.11 |
| ATOM | 13424 | C | LEU | 1995 | 30.514 | −44.507 | 28.924 | 1.00 | 15.56 |
| ATOM | 13425 | O | LEU | 1995 | 29.807 | −45.496 | 28.766 | 1.00 | 17.28 |
| ATOM | 13426 | N | ALA | 1996 | 30.739 | −43.942 | 30.106 | 1.00 | 15.73 |
| ATOM | 13427 | CA | ALA | 1996 | 30.128 | −44.435 | 31.324 | 1.00 | 15.27 |
| ATOM | 13428 | CB | ALA | 1996 | 30.946 | −44.020 | 32.541 | 1.00 | 15.65 |
| ATOM | 13429 | C | ALA | 1996 | 28.727 | −43.836 | 31.402 | 1.00 | 14.70 |
| ATOM | 13430 | O | ALA | 1996 | 27.795 | −44.485 | 31.871 | 1.00 | 14.43 |
| ATOM | 13431 | N | ILE | 1997 | 28.575 | −42.595 | 30.934 | 1.00 | 14.99 |
| ATOM | 13432 | CA | ILE | 1997 | 27.266 | −41.939 | 30.946 | 1.00 | 13.59 |
| ATOM | 13433 | CB | ILE | 1997 | 27.372 | −40.419 | 30.633 | 1.00 | 13.71 |
| ATOM | 13434 | CG2 | ILE | 1997 | 28.184 | −39.710 | 31.721 | 1.00 | 10.02 |
| ATOM | 13435 | CG1 | ILE | 1997 | 27.993 | −40.211 | 29.249 | 1.00 | 11.88 |
| ATOM | 13436 | CD1 | ILE | 1997 | 28.068 | −38.736 | 28.834 | 1.00 | 13.96 |
| ATOM | 13437 | C | ILE | 1997 | 26.337 | −42.565 | 29.909 | 1.00 | 12.60 |
| ATOM | 13438 | O | ILE | 1997 | 26.786 | −43.173 | 28.935 | 1.00 | 12.53 |
| ATOM | 13439 | N | PRO | 1998 | 25.018 | −42.431 | 30.111 | 1.00 | 13.25 |
| ATOM | 13440 | CD | PRO | 1998 | 24.297 | −41.930 | 31.296 | 1.00 | 10.70 |
| ATOM | 13441 | CA | PRO | 1998 | 24.106 | −43.019 | 29.128 | 1.00 | 12.65 |
| ATOM | 13442 | CB | PRO | 1998 | 22.745 | −42.952 | 29.824 | 1.00 | 13.84 |
| ATOM | 13443 | CG | PRO | 1998 | 22.895 | −41.813 | 30.788 | 1.00 | 18.97 |
| ATOM | 13444 | C | PRO | 1998 | 24.117 | −42.292 | 27.784 | 1.00 | 12.72 |
| ATOM | 13445 | O | PRO | 1998 | 24.340 | −41.081 | 27.718 | 1.00 | 12.80 |
| ATOM | 13446 | N | VAL | 1999 | 23.890 | −43.053 | 26.721 | 1.00 | 11.68 |
| ATOM | 13447 | CA | VAL | 1999 | 23.850 | −42.507 | 25.376 | 1.00 | 13.14 |
| ATOM | 13448 | CB | VAL | 1999 | 24.972 | −43.123 | 24.507 | 1.00 | 14.39 |
| ATOM | 13449 | CG1 | VAL | 1999 | 24.829 | −42.664 | 23.061 | 1.00 | 13.88 |
| ATOM | 13450 | CG2 | VAL | 1999 | 26.338 | −42.685 | 25.062 | 1.00 | 13.61 |
| ATOM | 13451 | C | VAL | 1999 | 22.483 | −42.815 | 24.784 | 1.00 | 12.68 |
| ATOM | 13452 | O | VAL | 1999 | 22.064 | −43.969 | 24.753 | 1.00 | 11.67 |
| ATOM | 13453 | N | ILE | 2000 | 21.788 | −41.768 | 24.346 | 1.00 | 12.00 |
| ATOM | 13454 | CA | ILE | 2000 | 20.449 | −41.870 | 23.772 | 1.00 | 10.42 |
| ATOM | 13455 | CB | ILE | 2000 | 19.527 | −40.764 | 24.362 | 1.00 | 11.32 |
| ATOM | 13456 | CG2 | ILE | 2000 | 18.190 | −40.765 | 23.645 | 1.00 | 14.54 |
| ATOM | 13457 | CG1 | ILE | 2000 | 19.350 | −40.977 | 25.869 | 1.00 | 12.67 |
| ATOM | 13458 | CD1 | ILE | 2000 | 18.671 | −39.808 | 26.580 | 1.00 | 14.64 |
| ATOM | 13459 | C | ILE | 2000 | 20.562 | −41.667 | 22.265 | 1.00 | 11.37 |
| ATOM | 13460 | O | ILE | 2000 | 21.140 | −40.682 | 21.814 | 1.00 | 12.18 |
| ATOM | 13461 | N | GLY | 2001 | 20.021 | −42.595 | 21.486 | 1.00 | 9.93 |
| ATOM | 13462 | CA | GLY | 2001 | 20.124 | −42.462 | 20.048 | 1.00 | 10.42 |
| ATOM | 13463 | C | GLY | 2001 | 18.845 | −42.223 | 19.274 | 1.00 | 10.95 |
| ATOM | 13464 | O | GLY | 2001 | 17.735 | −42.486 | 19.742 | 1.00 | 10.35 |
| ATOM | 13465 | N | ILE | 2002 | 19.024 | −41.667 | 18.085 | 1.00 | 12.38 |
| ATOM | 13466 | CA | ILE | 2002 | 17.945 | −41.430 | 17.149 | 1.00 | 12.82 |
| ATOM | 13467 | CB | ILE | 2002 | 17.347 | −39.975 | 17.254 | 1.00 | 15.11 |
| ATOM | 13468 | CG2 | ILE | 2002 | 18.439 | −38.929 | 17.272 | 1.00 | 17.13 |
| ATOM | 13469 | CG1 | ILE | 2002 | 16.384 | −39.726 | 16.101 | 1.00 | 16.48 |
| ATOM | 13470 | CD1 | ILE | 2002 | 15.206 | −40.620 | 16.103 | 1.00 | 21.78 |
| ATOM | 13471 | C | ILE | 2002 | 18.652 | −41.700 | 15.832 | 1.00 | 14.25 |
| ATOM | 13472 | O | ILE | 2002 | 19.612 | −41.019 | 15.465 | 1.00 | 14.32 |
| ATOM | 13473 | N | GLY | 2003 | 18.202 | −42.745 | 15.145 | 1.00 | 13.78 |
| ATOM | 13474 | CA | GLY | 2003 | 18.839 | −43.124 | 13.902 | 1.00 | 13.18 |
| ATOM | 13475 | C | GLY | 2003 | 20.243 | −43.651 | 14.160 | 1.00 | 13.92 |
| ATOM | 13476 | O | GLY | 2003 | 21.108 | −43.548 | 13.293 | 1.00 | 16.63 |
| ATOM | 13477 | N | ALA | 2004 | 20.485 | −44.208 | 15.347 | 1.00 | 14.85 |
| ATOM | 13478 | CA | ALA | 2004 | 21.803 | −44.758 | 15.677 | 1.00 | 15.30 |
| ATOM | 13479 | CB | ALA | 2004 | 22.427 | −43.972 | 16.832 | 1.00 | 16.97 |
| ATOM | 13480 | C | ALA | 2004 | 21.732 | −46.247 | 16.031 | 1.00 | 15.54 |
| ATOM | 13481 | O | ALA | 2004 | 22.688 | −46.814 | 16.565 | 1.00 | 18.51 |
| ATOM | 13482 | N | GLY | 2005 | 20.598 | −46.880 | 15.741 | 1.00 | 16.16 |
| ATOM | 13483 | CA | GLY | 2005 | 20.448 | −48.302 | 16.033 | 1.00 | 14.60 |
| ATOM | 13484 | C | GLY | 2005 | 20.079 | −48.614 | 17.473 | 1.00 | 15.86 |
| ATOM | 13485 | O | GLY | 2005 | 20.022 | −47.717 | 18.311 | 1.00 | 16.19 |
| ATOM | 13486 | N | ASN | 2006 | 19.843 | −49.891 | 17.773 | 1.00 | 14.74 |
| ATOM | 13487 | CA | ASN | 2006 | 19.454 | −50.290 | 19.130 | 1.00 | 14.82 |
| ATOM | 13488 | CB | ASN | 2006 | 18.552 | −51.530 | 19.083 | 1.00 | 14.04 |
| ATOM | 13489 | CG | ASN | 2006 | 19.300 | −52.803 | 18.712 | 1.00 | 12.52 |
| ATOM | 13490 | OD1 | ASN | 2006 | 18.768 | −53.901 | 18.870 | 1.00 | 16.70 |
| ATOM | 13491 | ND2 | ASN | 2006 | 20.521 | −52.668 | 18.222 | 1.00 | 12.06 |
| ATOM | 13492 | C | ASN | 2006 | 20.610 | −50.548 | 20.094 | 1.00 | 14.16 |
| ATOM | 13493 | O | ASN | 2006 | 20.403 | −51.082 | 21.183 | 1.00 | 15.13 |
| ATOM | 13494 | N | VAL | 2007 | 21.817 | −50.153 | 19.703 | 1.00 | 14.84 |
| ATOM | 13495 | CA | VAL | 2007 | 23.002 | −50.358 | 20.534 | 1.00 | 16.00 |
| ATOM | 13496 | CB | VAL | 2007 | 24.277 | −50.349 | 19.677 | 1.00 | 18.48 |
| ATOM | 13497 | CG1 | VAL | 2007 | 25.448 | −50.869 | 20.490 | 1.00 | 25.46 |
| ATOM | 13498 | CG2 | VAL | 2007 | 24.073 | −51.178 | 18.421 | 1.00 | 22.41 |
| ATOM | 13499 | C | VAL | 2007 | 23.167 | −49.310 | 21.639 | 1.00 | 16.08 |
| ATOM | 13500 | O | VAL | 2007 | 23.970 | −49.486 | 22.557 | 1.00 | 14.39 |
| ATOM | 13501 | N | THR | 2008 | 22.418 | −48.215 | 21.549 | 1.00 | 13.91 |
| ATOM | 13502 | CA | THR | 2008 | 22.509 | −47.158 | 22.557 | 1.00 | 13.64 |
| ATOM | 13503 | CB | THR | 2008 | 21.997 | −45.809 | 21.980 | 1.00 | 12.91 |
| ATOM | 13504 | OG1 | THR | 2008 | 20.692 | −45.980 | 21.412 | 1.00 | 13.88 |
| ATOM | 13505 | CG2 | THR | 2008 | 22.941 | −45.303 | 20.901 | 1.00 | 14.51 |
| ATOM | 13506 | C | THR | 2008 | 21.743 | −47.523 | 23.839 | 1.00 | 13.63 |
| ATOM | 13507 | O | THR | 2008 | 20.956 | −48.470 | 23.847 | 1.00 | 15.42 |
| ATOM | 13508 | N | ASP | 2009 | 21.993 | −46.796 | 24.925 | 1.00 | 13.00 |
| ATOM | 13509 | CA | ASP | 2009 | 21.329 | −47.071 | 26.205 | 1.00 | 12.92 |
| ATOM | 13510 | CB | ASP | 2009 | 22.064 | −46.350 | 27.337 | 1.00 | 13.27 |
| ATOM | 13511 | CG | ASP | 2009 | 23.552 | −46.681 | 27.365 | 1.00 | 14.17 |
| ATOM | 13512 | OD1 | ASP | 2009 | 23.898 | −47.849 | 27.650 | 1.00 | 15.47 |
| ATOM | 13513 | OD2 | ASP | 2009 | 24.362 | −45.779 | 27.093 | 1.00 | 14.16 |
| ATOM | 13514 | C | ASP | 2009 | 19.859 | −46.652 | 26.208 | 1.00 | 13.92 |
| ATOM | 13515 | O | ASP | 2009 | 19.035 | −47.205 | 26.943 | 1.00 | 14.59 |
| ATOM | 13516 | N | GLY | 2010 | 19.542 | −45.658 | 25.387 | 1.00 | 14.50 |
| ATOM | 13517 | CA | GLY | 2010 | 18.173 | −45.181 | 25.300 | 1.00 | 13.29 |
| ATOM | 13518 | C | GLY | 2010 | 17.829 | −44.764 | 23.878 | 1.00 | 11.32 |
| ATOM | 13519 | O | GLY | 2010 | 18.700 | −44.728 | 23.008 | 1.00 | 11.26 |
| ATOM | 13520 | N | GLN | 2011 | 16.562 | −44.437 | 23.644 | 1.00 | 11.11 |
| ATOM | 13521 | CA | GLN | 2011 | 16.095 | −44.032 | 22.325 | 1.00 | 10.95 |
| ATOM | 13522 | CB | GLN | 2011 | 15.215 | −45.121 | 21.706 | 1.00 | 12.17 |
| ATOM | 13523 | CG | GLN | 2011 | 15.926 | −46.426 | 21.378 | 1.00 | 10.38 |
| ATOM | 13524 | CD | GLN | 2011 | 16.987 | −46.262 | 20.303 | 1.00 | 9.79 |
| ATOM | 13525 | OE1 | GLN | 2011 | 16.812 | −45.508 | 19.344 | 1.00 | 12.91 |
| ATOM | 13526 | NE2 | GLN | 2011 | 18.086 | −46.986 | 20.445 | 1.00 | 12.26 |
| ATOM | 13527 | C | GLN | 2011 | 15.254 | −42.775 | 22.417 | 1.00 | 11.93 |
| ATOM | 13528 | O | GLN | 2011 | 14.646 | −42.495 | 23.451 | 1.00 | 9.84 |
| ATOM | 13529 | N | ILE | 2012 | 15.208 | −42.023 | 21.327 | 1.00 | 13.12 |
| ATOM | 13530 | CA | ILE | 2012 | 14.384 | −40.835 | 21.307 | 1.00 | 16.32 |
| ATOM | 13531 | CB | ILE | 2012 | 15.228 | −39.563 | 21.592 | 1.00 | 20.21 |
| ATOM | 13532 | CG2 | ILE | 2012 | 16.164 | −39.278 | 20.451 | 1.00 | 19.95 |
| ATOM | 13533 | CG1 | ILE | 2012 | 14.302 | −38.372 | 21.851 | 1.00 | 22.75 |
| ATOM | 13534 | CD1 | ILE | 2012 | 14.955 | −37.275 | 22.672 | 1.00 | 20.84 |
| ATOM | 13535 | C | ILE | 2012 | 13.700 | −40.764 | 19.951 | 1.00 | 18.75 |
| ATOM | 13536 | O | ILE | 2012 | 14.243 | −41.208 | 18.939 | 1.00 | 17.43 |
| ATOM | 13537 | N | LEU | 2013 | 12.479 | −40.250 | 19.942 | 1.00 | 19.42 |
| ATOM | 13538 | CA | LEU | 2013 | 11.746 | −40.110 | 18.700 | 1.00 | 23.65 |
| ATOM | 13539 | CB | LEU | 2013 | 10.914 | −41.356 | 18.419 | 1.00 | 26.87 |
| ATOM | 13540 | CG | LEU | 2013 | 11.228 | −42.102 | 17.114 | 1.00 | 28.56 |
| ATOM | 13541 | CD1 | LEU | 2013 | 10.179 | −43.189 | 16.952 | 1.00 | 27.78 |
| ATOM | 13542 | CD2 | LEU | 2013 | 11.206 | −41.148 | 15.900 | 1.00 | 28.97 |
| ATOM | 13543 | C | LEU | 2013 | 10.835 | −38.903 | 18.751 | 1.00 | 24.10 |
| ATOM | 13544 | O | LEU | 2013 | 10.387 | −38.489 | 19.830 | 1.00 | 19.94 |
| ATOM | 13545 | N | VAL | 2014 | 10.588 | −38.337 | 17.573 | 1.00 | 23.42 |
| ATOM | 13546 | CA | VAL | 2014 | 9.714 | −37.186 | 17.420 | 1.00 | 22.95 |
| ATOM | 13547 | CB | VAL | 2014 | 9.892 | −36.500 | 16.039 | 1.00 | 25.08 |
| ATOM | 13548 | CG1 | VAL | 2014 | 8.905 | −35.340 | 15.907 | 1.00 | 25.84 |
| ATOM | 13549 | CG2 | VAL | 2014 | 11.312 | −35.989 | 15.898 | 1.00 | 29.40 |
| ATOM | 13550 | C | VAL | 2014 | 8.310 | −37.732 | 17.499 | 1.00 | 19.50 |
| ATOM | 13551 | O | VAL | 2014 | 7.917 | −38.578 | 16.688 | 1.00 | 17.37 |
| ATOM | 13552 | N | MET | 2015 | 7.563 | −37.258 | 18.488 | 1.00 | 16.88 |
| ATOM | 13553 | CA | MET | 2015 | 6.199 | −37.712 | 18.687 | 1.00 | 15.75 |
| ATOM | 13554 | CB | MET | 2015 | 5.567 | −37.024 | 19.913 | 1.00 | 13.34 |
| ATOM | 13555 | CG | MET | 2015 | 5.461 | −35.519 | 19.841 | 1.00 | 14.95 |
| ATOM | 13556 | SD | MET | 2015 | 3.933 | −34.982 | 20.657 | 1.00 | 14.22 |
| ATOM | 13557 | CE | MET | 2015 | 2.778 | −35.272 | 19.370 | 1.00 | 13.27 |
| ATOM | 13558 | C | MET | 2015 | 5.340 | −37.485 | 17.451 | 1.00 | 15.30 |
| ATOM | 13559 | O | MET | 2015 | 4.460 | −38.288 | 17.150 | 1.00 | 16.99 |
| ATOM | 13560 | N | HIS | 2016 | 5.602 | −36.411 | 16.714 | 1.00 | 12.72 |
| ATOM | 13561 | CA | HIS | 2016 | 4.804 | −36.144 | 15.527 | 1.00 | 15.08 |
| ATOM | 13562 | CB | HIS | 2016 | 5.115 | −34.739 | 15.005 | 1.00 | 15.69 |
| ATOM | 13563 | CG | HIS | 2016 | 4.605 | −33.664 | 15.915 | 1.00 | 14.26 |
| ATOM | 13564 | CD2 | HIS | 2016 | 5.132 | −33.129 | 17.043 | 1.00 | 11.38 |
| ATOM | 13565 | ND1 | HIS | 2016 | 3.330 | −33.146 | 15.808 | 1.00 | 15.21 |
| ATOM | 13566 | CE1 | HIS | 2016 | 3.092 | −32.345 | 16.833 | 1.00 | 13.82 |
| ATOM | 13567 | NE2 | HIS | 2016 | 4.170 | −32.318 | 17.598 | 1.00 | 16.57 |
| ATOM | 13568 | C | HIS | 2016 | 4.989 | −37.221 | 14.462 | 1.00 | 16.85 |
| ATOM | 13569 | O | HIS | 2016 | 4.085 | −37.462 | 13.658 | 1.00 | 17.99 |
| ATOM | 13570 | N | ASP | 2017 | 6.143 | −37.883 | 14.455 | 1.00 | 17.44 |
| ATOM | 13571 | CA | ASP | 2017 | 6.359 | −38.967 | 13.495 | 1.00 | 22.03 |
| ATOM | 13572 | CB | ASP | 2017 | 7.850 | −39.142 | 13.177 | 1.00 | 23.41 |
| ATOM | 13573 | CG | ASP | 2017 | 8.440 | −37.954 | 12.431 | 1.00 | 27.95 |
| ATOM | 13574 | OD1 | ASP | 2017 | 7.857 | −37.548 | 11.409 | 1.00 | 27.29 |
| ATOM | 13575 | OD2 | ASP | 2017 | 9.488 | −37.442 | 12.873 | 1.00 | 26.63 |
| ATOM | 13576 | C | ASP | 2017 | 5.817 | −40.278 | 14.079 | 1.00 | 21.90 |
| ATOM | 13577 | O | ASP | 2017 | 5.254 | −41.105 | 13.357 | 1.00 | 23.38 |
| ATOM | 13578 | N | ALA | 2018 | 5.986 | −40.454 | 15.388 | 1.00 | 22.26 |
| ATOM | 13579 | CA | ALA | 2018 | 5.538 | −41.661 | 16.083 | 1.00 | 22.32 |
| ATOM | 13580 | CB | ALA | 2018 | 5.940 | −41.585 | 17.559 | 1.00 | 23.61 |
| ATOM | 13581 | C | ALA | 2018 | 4.037 | −41.923 | 15.959 | 1.00 | 24.21 |
| ATOM | 13582 | O | ALA | 2018 | 3.597 | −43.064 | 16.085 | 1.00 | 25.32 |
| ATOM | 13583 | N | PHE | 2019 | 3.255 | −40.871 | 15.711 | 1.00 | 21.91 |
| ATOM | 13584 | CA | PHE | 2019 | 1.807 | −41.011 | 15.561 | 1.00 | 22.15 |
| ATOM | 13585 | CB | PHE | 2019 | 1.080 | −40.151 | 16.596 | 1.00 | 23.71 |
| ATOM | 13586 | CG | PHE | 2019 | 1.580 | −40.355 | 17.999 | 1.00 | 24.13 |
| ATOM | 13587 | CD1 | PHE | 2019 | 1.717 | −41.638 | 18.516 | 1.00 | 24.21 |
| ATOM | 13588 | CD2 | PHE | 2019 | 1.935 | −39.271 | 18.795 | 1.00 | 25.21 |
| ATOM | 13589 | CE1 | PHE | 2019 | 2.207 | −41.843 | 19.807 | 1.00 | 25.08 |
| ATOM | 13590 | CE2 | PHE | 2019 | 2.426 | −39.463 | 20.091 | 1.00 | 25.89 |
| ATOM | 13591 | CZ | PHE | 2019 | 2.561 | −40.752 | 20.593 | 1.00 | 26.24 |
| ATOM | 13592 | C | PHE | 2019 | 1.329 | −40.637 | 14.155 | 1.00 | 21.96 |
| ATOM | 13593 | O | PHE | 2019 | 0.156 | −40.325 | 13.947 | 1.00 | 22.51 |
| ATOM | 13594 | N | GLY | 2020 | 2.243 | −40.658 | 13.194 | 1.00 | 23.05 |
| ATOM | 13595 | CA | GLY | 2020 | 1.879 | −40.328 | 11.829 | 1.00 | 24.29 |
| ATOM | 13596 | C | GLY | 2020 | 1.192 | −38.985 | 11.660 | 1.00 | 23.30 |
| ATOM | 13597 | O | GLY | 2020 | 0.433 | −38.791 | 10.706 | 1.00 | 24.31 |
| ATOM | 13598 | N | ILE | 2021 | 1.445 | −38.049 | 12.570 | 1.00 | 22.12 |
| ATOM | 13599 | CA | ILE | 2021 | 0.822 | −36.731 | 12.466 | 1.00 | 19.93 |
| ATOM | 13600 | CB | ILE | 2021 | 0.976 | −35.919 | 13.792 | 1.00 | 18.55 |
| ATOM | 13601 | CG2 | ILE | 2021 | 0.483 | −34.500 | 13.595 | 1.00 | 18.65 |
| ATOM | 13602 | CG1 | ILE | 2021 | 0.168 | −36.590 | 14.912 | 1.00 | 17.73 |
| ATOM | 13603 | CD1 | ILE | 2021 | 0.485 | −36.084 | 16.319 | 1.00 | 15.80 |
| ATOM | 13604 | C | ILE | 2021 | 1.447 | −35.961 | 11.300 | 1.00 | 20.77 |
| ATOM | 13605 | O | ILE | 2021 | 0.738 | −35.348 | 10.502 | 1.00 | 18.99 |
| ATOM | 13606 | N | THR | 2022 | 2.771 | −36.008 | 11.194 | 1.00 | 22.40 |
| ATOM | 13607 | CA | THR | 2022 | 3.473 | −35.313 | 10.120 | 1.00 | 25.87 |
| ATOM | 13608 | CB | THR | 2022 | 4.985 | −35.269 | 10.385 | 1.00 | 27.00 |
| ATOM | 13609 | OG1 | THR | 2022 | 5.517 | −36.600 | 10.373 | 1.00 | 32.73 |
| ATOM | 13610 | CG2 | THR | 2022 | 5.262 | −34.641 | 11.735 | 1.00 | 26.74 |
| ATOM | 13611 | C | THR | 2022 | 3.240 | −35.978 | 8.765 | 1.00 | 27.71 |
| ATOM | 13612 | O | THR | 2022 | 3.208 | −37.208 | 8.663 | 1.00 | 26.18 |
| ATOM | 13613 | N | GLY | 2023 | 3.078 | −35.147 | 7.738 | 1.00 | 29.75 |
| ATOM | 13614 | CA | GLY | 2023 | 2.854 | −35.620 | 6.381 | 1.00 | 36.68 |
| ATOM | 13615 | C | GLY | 2023 | 2.624 | −37.111 | 6.213 | 1.00 | 40.05 |
| ATOM | 13616 | O | GLY | 2023 | 1.665 | −37.666 | 6.754 | 1.00 | 41.97 |
| ATOM | 13617 | N | GLY | 2024 | 3.503 | −37.765 | 5.458 | 1.00 | 41.61 |
| ATOM | 13618 | CA | GLY | 2024 | 3.364 | −39.194 | 5.240 | 1.00 | 43.00 |
| ATOM | 13619 | C | GLY | 2024 | 4.675 | −39.899 | 4.961 | 1.00 | 43.33 |
| ATOM | 13620 | O | GLY | 2024 | 4.750 | −41.125 | 5.012 | 1.00 | 44.64 |
| ATOM | 13621 | N | HIS | 2025 | 5.712 | −39.125 | 4.664 | 1.00 | 42.93 |
| ATOM | 13622 | CA | HIS | 2025 | 7.031 | −39.680 | 4.374 | 1.00 | 42.66 |
| ATOM | 13623 | CB | HIS | 2025 | 7.622 | −38.976 | 3.151 | 1.00 | 46.48 |
| ATOM | 13624 | CG | HIS | 2025 | 7.432 | −37.489 | 3.161 | 1.00 | 50.68 |
| ATOM | 13625 | CD2 | HIS | 2025 | 6.729 | −36.676 | 2.337 | 1.00 | 52.17 |
| ATOM | 13626 | ND1 | HIS | 2025 | 8.001 | −36.669 | 4.112 | 1.00 | 52.20 |
| ATOM | 13627 | CE1 | HIS | 2025 | 7.660 | −35.415 | 3.872 | 1.00 | 52.74 |
| ATOM | 13628 | NE2 | HIS | 2025 | 6.888 | −35.392 | 2.801 | 1.00 | 53.69 |
| ATOM | 13629 | C | HIS | 2025 | 7.983 | −39.548 | 5.563 | 1.00 | 39.08 |
| ATOM | 13630 | O | HIS | 2025 | 8.882 | −38.709 | 5.552 | 1.00 | 38.93 |
| ATOM | 13631 | N | ILE | 2026 | 7.790 | −40.381 | 6.582 | 1.00 | 35.71 |
| ATOM | 13632 | CA | ILE | 2026 | 8.638 | −40.330 | 7.771 | 1.00 | 32.13 |
| ATOM | 13633 | CB | ILE | 2026 | 8.040 | −41.148 | 8.927 | 1.00 | 33.92 |
| ATOM | 13634 | CG2 | ILE | 2026 | 6.670 | −40.599 | 9.305 | 1.00 | 35.60 |
| ATOM | 13635 | CG1 | ILE | 2026 | 7.952 | −42.620 | 8.522 | 1.00 | 33.22 |
| ATOM | 13636 | CD1 | ILE | 2026 | 7.704 | −43.554 | 9.685 | 1.00 | 34.59 |
| ATOM | 13637 | C | ILE | 2026 | 10.045 | −40.857 | 7.509 | 1.00 | 27.67 |
| ATOM | 13638 | O | ILE | 2026 | 10.252 | −41.720 | 6.652 | 1.00 | 27.84 |
| ATOM | 13639 | N | PRO | 2027 | 11.035 | −40.346 | 8.256 | 1.00 | 25.23 |
| ATOM | 13640 | CD | PRO | 2027 | 10.945 | −39.349 | 9.333 | 1.00 | 23.72 |
| ATOM | 13641 | CA | PRO | 2027 | 12.417 | −40.793 | 8.075 | 1.00 | 23.00 |
| ATOM | 13642 | CB | PRO | 2027 | 13.189 | −39.972 | 9.111 | 1.00 | 24.44 |
| ATOM | 13643 | CG | PRO | 2027 | 12.159 | −39.676 | 10.159 | 1.00 | 26.02 |
| ATOM | 13644 | C | PRO | 2027 | 12.596 | −42.299 | 8.266 | 1.00 | 21.46 |
| ATOM | 13645 | O | PRO | 2027 | 11.814 | −42.950 | 8.964 | 1.00 | 18.01 |
| ATOM | 13646 | N | LYS | 2028 | 13.638 | −42.840 | 7.642 | 1.00 | 21.15 |
| ATOM | 13647 | CA | LYS | 2028 | 13.928 | −44.266 | 7.722 | 1.00 | 22.60 |
| ATOM | 13648 | CB | LYS | 2028 | 15.189 | −44.607 | 6.914 | 1.00 | 25.69 |
| ATOM | 13649 | CG | LYS | 2028 | 14.946 | −44.928 | 5.441 | 1.00 | 33.29 |
| ATOM | 13650 | CD | LYS | 2028 | 14.455 | −43.717 | 4.653 | 1.00 | 36.85 |
| ATOM | 13651 | CE | LYS | 2028 | 14.326 | −44.047 | 3.170 | 1.00 | 40.01 |
| ATOM | 13652 | NZ | LYS | 2028 | 14.001 | −42.848 | 2.343 | 1.00 | 41.13 |
| ATOM | 13653 | C | LYS | 2028 | 14.109 | −44.767 | 9.146 | 1.00 | 20.12 |
| ATOM | 13654 | O | LYS | 2028 | 13.751 | −45.901 | 9.448 | 1.00 | 19.79 |
| ATOM | 13655 | N | PHE | 2029 | 14.656 | −43.926 | 10.020 | 1.00 | 18.98 |
| ATOM | 13656 | CA | PHE | 2029 | 14.904 | −44.337 | 11.400 | 1.00 | 17.08 |
| ATOM | 13657 | CB | PHE | 2029 | 16.031 | −43.494 | 11.994 | 1.00 | 16.61 |
| ATOM | 13658 | CG | PHE | 2029 | 15.756 | −42.027 | 11.973 | 1.00 | 18.88 |
| ATOM | 13659 | CD1 | PHE | 2029 | 14.872 | −41.459 | 12.882 | 1.00 | 18.51 |
| ATOM | 13660 | CD2 | PHE | 2029 | 16.353 | −41.211 | 11.014 | 1.00 | 18.71 |
| ATOM | 13661 | CE1 | PHE | 2029 | 14.585 | −40.097 | 12.842 | 1.00 | 19.42 |
| ATOM | 13662 | CE2 | PHE | 2029 | 16.072 | −39.846 | 10.961 | 1.00 | 19.33 |
| ATOM | 13663 | CZ | PHE | 2029 | 15.187 | −39.288 | 11.875 | 1.00 | 19.51 |
| ATOM | 13664 | C | PHE | 2029 | 13.683 | −44.279 | 12.313 | 1.00 | 14.56 |
| ATOM | 13665 | O | PHE | 2029 | 13.732 | −44.765 | 13.435 | 1.00 | 12.75 |
| ATOM | 13666 | N | ALA | 2030 | 12.587 | −43.706 | 11.822 | 1.00 | 13.77 |
| ATOM | 13667 | CA | ALA | 2030 | 11.374 | −43.579 | 12.615 | 1.00 | 13.91 |
| ATOM | 13668 | CB | ALA | 2030 | 10.752 | −42.221 | 12.387 | 1.00 | 14.25 |
| ATOM | 13669 | C | ALA | 2030 | 10.336 | −44.661 | 12.345 | 1.00 | 15.51 |
| ATOM | 13670 | O | ALA | 2030 | 10.415 | −45.389 | 11.356 | 1.00 | 14.24 |
| ATOM | 13671 | N | LYS | 2031 | 9.360 | −44.751 | 13.243 | 1.00 | 13.26 |
| ATOM | 13672 | CA | LYS | 2031 | 8.287 | −45.713 | 13.122 | 1.00 | 13.53 |
| ATOM | 13673 | CB | LYS | 2031 | 8.599 | −46.980 | 13.924 | 1.00 | 13.73 |
| ATOM | 13674 | CG | LYS | 2031 | 7.469 | −48.008 | 13.894 | 1.00 | 13.98 |
| ATOM | 13675 | CD | LYS | 2031 | 7.871 | −49.321 | 14.566 | 1.00 | 15.44 |
| ATOM | 13676 | CE | LYS | 2031 | 6.659 | −50.206 | 14.782 | 1.00 | 17.20 |
| ATOM | 13677 | NZ | LYS | 2031 | 7.004 | −51.532 | 15.354 | 1.00 | 18.85 |
| ATOM | 13678 | C | LYS | 2031 | 6.996 | −45.096 | 13.625 | 1.00 | 13.24 |
| ATOM | 13679 | O | LYS | 2031 | 6.981 | −44.383 | 14.633 | 1.00 | 12.44 |
| ATOM | 13680 | N | ASN | 2032 | 5.915 | −45.359 | 12.900 | 1.00 | 13.87 |
| ATOM | 13681 | CA | ASN | 2032 | 4.594 | −44.866 | 13.272 | 1.00 | 14.13 |
| ATOM | 13682 | CB | ASN | 2032 | 3.746 | −44.625 | 12.015 | 1.00 | 15.58 |
| ATOM | 13683 | CG | ASN | 2032 | 2.318 | −44.218 | 12.336 | 1.00 | 17.14 |
| ATOM | 13684 | OD1 | ASN | 2032 | 1.846 | −44.384 | 13.465 | 1.00 | 18.37 |
| ATOM | 13685 | ND2 | ASN | 2032 | 1.614 | −43.699 | 11.338 | 1.00 | 15.68 |
| ATOM | 13686 | C | ASN | 2032 | 3.964 | −45.964 | 14.124 | 1.00 | 14.09 |
| ATOM | 13687 | O | ASN | 2032 | 3.512 | −46.987 | 13.594 | 1.00 | 13.04 |
| ATOM | 13688 | N | PHE | 2033 | 3.955 | −45.760 | 15.435 | 1.00 | 11.21 |
| ATOM | 13689 | CA | PHE | 2033 | 3.382 | −46.733 | 16.370 | 1.00 | 13.08 |
| ATOM | 13690 | CB | PHE | 2033 | 3.967 | −46.540 | 17.778 | 1.00 | 13.19 |
| ATOM | 13691 | CG | PHE | 2033 | 5.412 | −46.946 | 17.888 | 1.00 | 13.28 |
| ATOM | 13692 | CD1 | PHE | 2033 | 6.430 | −46.051 | 17.569 | 1.00 | 13.02 |
| ATOM | 13693 | CD2 | PHE | 2033 | 5.754 | −48.254 | 18.240 | 1.00 | 12.37 |
| ATOM | 13694 | CE1 | PHE | 2033 | 7.773 | −46.446 | 17.587 | 1.00 | 14.04 |
| ATOM | 13695 | CE2 | PHE | 2033 | 7.091 | −48.663 | 18.262 | 1.00 | 11.80 |
| ATOM | 13696 | CZ | PHE | 2033 | 8.106 | −47.756 | 17.934 | 1.00 | 12.01 |
| ATOM | 13697 | C | PHE | 2033 | 1.864 | −46.643 | 16.421 | 1.00 | 14.14 |
| ATOM | 13698 | O | PHE | 2033 | 1.183 | −47.603 | 16.788 | 1.00 | 15.64 |
| ATOM | 13699 | N | LEU | 2034 | 1.315 | −45.493 | 16.054 | 1.00 | 13.12 |
| ATOM | 13700 | CA | LEU | 2034 | −0.135 | −45.372 | 16.075 | 1.00 | 14.61 |
| ATOM | 13701 | CB | LEU | 2034 | −0.566 | −43.933 | 15.780 | 1.00 | 15.30 |
| ATOM | 13702 | CG | LEU | 2034 | −2.088 | −43.744 | 15.737 | 1.00 | 15.00 |
| ATOM | 13703 | CD1 | LEU | 2034 | −2.712 | −44.235 | 17.047 | 1.00 | 17.20 |
| ATOM | 13704 | CD2 | LEU | 2034 | −2.419 | −42.283 | 15.484 | 1.00 | 15.32 |
| ATOM | 13705 | C | LEU | 2034 | −0.741 | −46.331 | 15.039 | 1.00 | 16.57 |
| ATOM | 13706 | O | LEU | 2034 | −1.736 | −47.004 | 15.313 | 1.00 | 14.84 |
| ATOM | 13707 | N | ALA | 2035 | −0.132 | −46.390 | 13.858 | 1.00 | 20.33 |
| ATOM | 13708 | CA | ALA | 2035 | −0.603 | −47.266 | 12.783 | 1.00 | 27.79 |
| ATOM | 13709 | CB | ALA | 2035 | 0.385 | −47.238 | 11.614 | 1.00 | 29.37 |
| ATOM | 13710 | C | ALA | 2035 | −0.790 | −48.700 | 13.269 | 1.00 | 33.38 |
| ATOM | 13711 | O | ALA | 2035 | −1.530 | −49.483 | 12.664 | 1.00 | 36.55 |
| ATOM | 13712 | N | GLU | 2036 | −0.117 | −49.036 | 14.366 | 1.00 | 37.14 |
| ATOM | 13713 | CA | GLU | 2036 | −0.194 | −50.373 | 14.954 | 1.00 | 41.95 |
| ATOM | 13714 | CB | GLU | 2036 | 0.893 | −50.541 | 16.026 | 1.00 | 42.91 |
| ATOM | 13715 | CG | GLU | 2036 | 2.290 | −50.150 | 15.573 | 1.00 | 47.48 |
| ATOM | 13716 | CD | GLU | 2036 | 2.844 | −51.058 | 14.493 | 1.00 | 49.09 |
| ATOM | 13717 | OE1 | GLU | 2036 | 3.892 | −50.704 | 13.909 | 1.00 | 50.73 |
| ATOM | 13718 | OE2 | GLU | 2036 | 2.240 | −52.125 | 14.231 | 1.00 | 51.86 |
| ATOM | 13719 | C | GLU | 2036 | −1.561 | −50.605 | 15.591 | 1.00 | 43.11 |
| ATOM | 13720 | O | GLU | 2036 | −1.915 | −51.740 | 15.916 | 1.00 | 44.60 |
| ATOM | 13721 | N | THR | 2037 | −2.313 | −49.520 | 15.770 | 1.00 | 43.75 |
| ATOM | 13722 | CA | THR | 2037 | −3.647 | −49.569 | 16.372 | 1.00 | 43.51 |
| ATOM | 13723 | CB | THR | 2037 | −3.554 | −49.742 | 17.894 | 1.00 | 44.86 |
| ATOM | 13724 | OG1 | THR | 2037 | −4.872 | −49.832 | 18.452 | 1.00 | 46.90 |
| ATOM | 13725 | CG2 | THR | 2037 | −2.821 | −48.567 | 18.513 | 1.00 | 47.17 |
| ATOM | 13726 | C | THR | 2037 | −4.422 | −48.283 | 16.075 | 1.00 | 41.90 |
| ATOM | 13727 | O | THR | 2037 | −4.206 | −47.644 | 15.046 | 1.00 | 43.72 |
| ATOM | 13728 | N | GLY | 2038 | −5.330 | −47.916 | 16.976 | 1.00 | 38.86 |
| ATOM | 13729 | CA | GLY | 2038 | −6.107 | −46.700 | 16.809 | 1.00 | 32.91 |
| ATOM | 13730 | C | GLY | 2038 | −6.187 | −45.946 | 18.123 | 1.00 | 29.01 |
| ATOM | 13731 | O | GLY | 2038 | −7.129 | −45.193 | 18.383 | 1.00 | 29.97 |
| ATOM | 13732 | N | ASP | 2039 | −5.184 | −46.157 | 18.964 | 1.00 | 23.77 |
| ATOM | 13733 | CA | ASP | 2039 | −5.134 | −45.510 | 20.271 | 1.00 | 19.97 |
| ATOM | 13734 | CB | ASP | 2039 | −5.582 | −46.503 | 21.353 | 1.00 | 23.10 |
| ATOM | 13735 | CG | ASP | 2039 | −5.458 | −45.935 | 22.755 | 1.00 | 26.45 |
| ATOM | 13736 | OD1 | ASP | 2039 | −4.352 | −45.993 | 23.316 | 1.00 | 25.80 |
| ATOM | 13737 | OD2 | ASP | 2039 | −6.466 | −45.412 | 23.284 | 1.00 | 33.68 |
| ATOM | 13738 | C | ASP | 2039 | −3.709 | −45.026 | 20.528 | 1.00 | 15.18 |
| ATOM | 13739 | O | ASP | 2039 | −2.770 | −45.814 | 20.510 | 1.00 | 11.93 |
| ATOM | 13740 | N | ILE | 2040 | −3.562 | −43.725 | 20.762 | 1.00 | 14.13 |
| ATOM | 13741 | CA | ILE | 2040 | −2.256 | −43.121 | 20.989 | 1.00 | 12.51 |
| ATOM | 13742 | CB | ILE | 2040 | −2.379 | −41.579 | 21.053 | 1.00 | 12.90 |
| ATOM | 13743 | CG2 | ILE | 2040 | −1.046 | −40.954 | 21.421 | 1.00 | 12.00 |
| ATOM | 13744 | CG1 | ILE | 2040 | −2.842 | −41.052 | 19.689 | 1.00 | 13.88 |
| ATOM | 13745 | CD1 | ILE | 2040 | −3.192 | −39.555 | 19.673 | 1.00 | 14.99 |
| ATOM | 13746 | C | ILE | 2040 | −1.544 | −43.645 | 22.234 | 1.00 | 12.95 |
| ATOM | 13747 | O | ILE | 2040 | −0.336 | −43.838 | 22.214 | 1.00 | 13.26 |
| ATOM | 13748 | N | ARG | 2041 | −2.280 | −43.881 | 23.312 | 1.00 | 11.80 |
| ATOM | 13749 | CA | ARG | 2041 | −1.657 | −44.390 | 24.520 | 1.00 | 13.04 |
| ATOM | 13750 | CB | ARG | 2041 | −2.667 | −44.389 | 25.660 | 1.00 | 13.92 |
| ATOM | 13751 | CG | ARG | 2041 | −2.981 | −42.985 | 26.125 | 1.00 | 14.21 |
| ATOM | 13752 | CD | ARG | 2041 | −4.050 | −42.968 | 27.178 | 1.00 | 16.52 |
| ATOM | 13753 | NE | ARG | 2041 | −4.194 | −41.629 | 27.733 | 1.00 | 14.95 |
| ATOM | 13754 | CZ | ARG | 2041 | −4.651 | −40.579 | 27.057 | 1.00 | 18.50 |
| ATOM | 13755 | NH1 | ARG | 2041 | −5.024 | −40.700 | 25.791 | 1.00 | 14.68 |
| ATOM | 13756 | NH2 | ARG | 2041 | −4.728 | −39.397 | 27.646 | 1.00 | 17.59 |
| ATOM | 13757 | C | ARG | 2041 | −1.113 | −45.793 | 24.264 | 1.00 | 12.55 |
| ATOM | 13758 | O | ARG | 2041 | −0.026 | −46.143 | 24.729 | 1.00 | 11.32 |
| ATOM | 13759 | N | ALA | 2042 | −1.862 | −46.598 | 23.515 | 1.00 | 12.58 |
| ATOM | 13760 | CA | ALA | 2042 | −1.405 | −47.946 | 23.186 | 1.00 | 13.09 |
| ATOM | 13761 | CB | ALA | 2042 | −2.481 | −48.698 | 22.387 | 1.00 | 13.73 |
| ATOM | 13762 | C | ALA | 2042 | −0.115 | −47.838 | 22.368 | 1.00 | 12.86 |
| ATOM | 13763 | O | ALA | 2042 | 0.823 | −48.625 | 22.554 | 1.00 | 13.69 |
| ATOM | 13764 | N | ALA | 2043 | −0.068 | −46.862 | 21.462 | 1.00 | 11.53 |
| ATOM | 13765 | CA | ALA | 2043 | 1.121 | −46.648 | 20.629 | 1.00 | 11.50 |
| ATOM | 13766 | CB | ALA | 2043 | 0.863 | −45.552 | 19.605 | 1.00 | 10.75 |
| ATOM | 13767 | C | ALA | 2043 | 2.322 | −46.277 | 21.491 | 1.00 | 10.66 |
| ATOM | 13768 | O | ALA | 2043 | 3.439 | −46.727 | 21.235 | 1.00 | 12.15 |
| ATOM | 13769 | N | VAL | 2044 | 2.093 | −45.449 | 22.505 | 1.00 | 9.56 |
| ATOM | 13770 | CA | VAL | 2044 | 3.166 | −45.060 | 23.410 | 1.00 | 9.69 |
| ATOM | 13771 | CB | VAL | 2044 | 2.684 | −43.988 | 24.419 | 1.00 | 11.94 |
| ATOM | 13772 | CG1 | VAL | 2044 | 3.702 | −43.828 | 25.536 | 1.00 | 11.70 |
| ATOM | 13773 | CG2 | VAL | 2044 | 2.506 | −42.656 | 23.694 | 1.00 | 9.79 |
| ATOM | 13774 | C | VAL | 2044 | 3.695 | −46.278 | 24.163 | 1.00 | 11.89 |
| ATOM | 13775 | O | VAL | 2044 | 4.910 | −46.478 | 24.268 | 1.00 | 11.33 |
| ATOM | 13776 | N | ARG | 2045 | 2.782 | −47.101 | 24.672 | 1.00 | 10.73 |
| ATOM | 13777 | CA | ARG | 2045 | 3.178 | −48.301 | 25.409 | 1.00 | 12.45 |
| ATOM | 13778 | CB | ARG | 2045 | 1.939 | −49.016 | 25.956 | 1.00 | 11.81 |
| ATOM | 13779 | CG | ARG | 2045 | 1.325 | −48.297 | 27.150 | 1.00 | 15.71 |
| ATOM | 13780 | CD | ARG | 2045 | 0.359 | −49.185 | 27.908 | 1.00 | 17.80 |
| ATOM | 13781 | NE | ARG | 2045 | −0.791 | −49.608 | 27.115 | 1.00 | 18.46 |
| ATOM | 13782 | CZ | ARG | 2045 | −1.866 | −48.862 | 26.874 | 1.00 | 19.90 |
| ATOM | 13783 | NH1 | ARG | 2045 | −1.955 | −47.632 | 27.360 | 1.00 | 18.74 |
| ATOM | 13784 | NH2 | ARG | 2045 | −2.874 | −49.358 | 26.165 | 1.00 | 17.78 |
| ATOM | 13785 | C | ARG | 2045 | 3.986 | −49.265 | 24.547 | 1.00 | 11.67 |
| ATOM | 13786 | O | ARG | 2045 | 4.925 | −49.897 | 25.031 | 1.00 | 13.65 |
| ATOM | 13787 | N | GLN | 2046 | 3.604 | −49.373 | 23.278 | 1.00 | 11.60 |
| ATOM | 13788 | CA | GLN | 2046 | 4.279 | −50.255 | 22.343 | 1.00 | 13.89 |
| ATOM | 13789 | CB | GLN | 2046 | 3.503 | −50.340 | 21.027 | 1.00 | 15.05 |
| ATOM | 13790 | CG | GLN | 2046 | 4.052 | −51.389 | 20.066 | 1.00 | 17.14 |
| ATOM | 13791 | CD | GLN | 2046 | 3.320 | −51.419 | 18.737 | 1.00 | 21.49 |
| ATOM | 13792 | OE1 | GLN | 2046 | 2.098 | −51.286 | 18.687 | 1.00 | 24.59 |
| ATOM | 13793 | NE2 | GLN | 2046 | 4.063 | −51.607 | 17.656 | 1.00 | 23.02 |
| ATOM | 13794 | C | GLN | 2046 | 5.682 | −49.742 | 22.071 | 1.00 | 14.22 |
| ATOM | 13795 | O | GLN | 2046 | 6.624 | −50.523 | 21.932 | 1.00 | 13.87 |
| ATOM | 13796 | N | TYR | 2047 | 5.821 | −48.424 | 21.982 | 1.00 | 12.66 |
| ATOM | 13797 | CA | TYR | 2047 | 7.136 | −47.834 | 21.740 | 1.00 | 13.34 |
| ATOM | 13798 | CB | TYR | 2047 | 6.993 | −46.321 | 21.552 | 1.00 | 12.45 |
| ATOM | 13799 | CG | TYR | 2047 | 8.285 | −45.548 | 21.643 | 1.00 | 13.33 |
| ATOM | 13800 | CD1 | TYR | 2047 | 9.397 | −45.883 | 20.855 | 1.00 | 12.66 |
| ATOM | 13801 | CE1 | TYR | 2047 | 10.582 | −45.148 | 20.940 | 1.00 | 15.89 |
| ATOM | 13802 | CD2 | TYR | 2047 | 8.392 | −44.469 | 22.510 | 1.00 | 12.07 |
| ATOM | 13803 | CE2 | TYR | 2047 | 9.557 | −43.735 | 22.601 | 1.00 | 14.07 |
| ATOM | 13804 | CZ | TYR | 2047 | 10.649 | −44.068 | 21.824 | 1.00 | 14.94 |
| ATOM | 13805 | OH | TYR | 2047 | 11.805 | −43.318 | 21.929 | 1.00 | 16.90 |
| ATOM | 13806 | C | TYR | 2047 | 8.030 | −48.163 | 22.939 | 1.00 | 12.59 |
| ATOM | 13807 | O | TYR | 2047 | 9.145 | −48.661 | 22.784 | 1.00 | 10.64 |
| ATOM | 13808 | N | MET | 2048 | 7.522 | −47.896 | 24.139 | 1.00 | 12.77 |
| ATOM | 13809 | CA | MET | 2048 | 8.266 | −48.190 | 25.361 | 1.00 | 13.01 |
| ATOM | 13810 | CB | MET | 2048 | 7.394 | −47.907 | 26.595 | 1.00 | 14.40 |
| ATOM | 13811 | CG | MET | 2048 | 7.101 | −46.434 | 26.842 | 1.00 | 14.98 |
| ATOM | 13812 | SD | MET | 2048 | 5.856 | −46.160 | 28.106 | 1.00 | 18.28 |
| ATOM | 13813 | CE | MET | 2048 | 6.752 | −46.711 | 29.577 | 1.00 | 18.21 |
| ATOM | 13814 | C | MET | 2048 | 8.730 | −49.647 | 25.400 | 1.00 | 14.55 |
| ATOM | 13815 | O | MET | 2048 | 9.907 | −49.941 | 25.652 | 1.00 | 11.07 |
| ATOM | 13816 | N | ALA | 2049 | 7.793 | −50.561 | 25.163 | 1.00 | 13.04 |
| ATOM | 13817 | CA | ALA | 2049 | 8.096 | −51.988 | 25.182 | 1.00 | 14.89 |
| ATOM | 13818 | CB | ALA | 2049 | 6.798 | −52.799 | 24.992 | 1.00 | 14.38 |
| ATOM | 13819 | C | ALA | 2049 | 9.128 | −52.385 | 24.130 | 1.00 | 15.06 |
| ATOM | 13820 | O | ALA | 2049 | 10.043 | −53.163 | 24.414 | 1.00 | 14.34 |
| ATOM | 13821 | N | GLU | 2050 | 9.008 | −51.849 | 22.916 | 1.00 | 12.60 |
| ATOM | 13822 | CA | GLU | 2050 | 9.963 | −52.224 | 21.869 | 1.00 | 13.77 |
| ATOM | 13823 | CB | GLU | 2050 | 9.461 | −51.778 | 20.480 | 1.00 | 10.52 |
| ATOM | 13824 | CG | GLU | 2050 | 8.388 | −52.697 | 19.915 | 1.00 | 12.90 |
| ATOM | 13825 | CD | GLU | 2050 | 7.895 | −52.284 | 18.544 | 1.00 | 14.19 |
| ATOM | 13826 | OE1 | GLU | 2050 | 8.707 | −51.760 | 17.752 | 1.00 | 14.22 |
| ATOM | 13827 | OE2 | GLU | 2050 | 6.695 | −52.502 | 18.255 | 1.00 | 13.57 |
| ATOM | 13828 | C | GLU | 2050 | 11.361 | −51.689 | 22.145 | 1.00 | 14.61 |
| ATOM | 13829 | O | GLU | 2050 | 12.354 | −52.322 | 21.790 | 1.00 | 12.04 |
| ATOM | 13830 | N | VAL | 2051 | 11.453 | −50.528 | 22.783 | 1.00 | 14.04 |
| ATOM | 13831 | CA | VAL | 2051 | 12.774 | −49.999 | 23.088 | 1.00 | 12.24 |
| ATOM | 13832 | CB | VAL | 2051 | 12.701 | −48.552 | 23.589 | 1.00 | 12.65 |
| ATOM | 13833 | CG1 | VAL | 2051 | 14.041 | −43.153 | 24.216 | 1.00 | 12.32 |
| ATOM | 13834 | CG2 | VAL | 2051 | 12.361 | −47.621 | 22.427 | 1.00 | 10.30 |
| ATOM | 13835 | C | VAL | 2051 | 13.434 | −50.866 | 24.159 | 1.00 | 12.70 |
| ATOM | 13836 | O | VAL | 2051 | 14.618 | −51.203 | 24.063 | 1.00 | 14.76 |
| ATOM | 13837 | N | GLU | 2052 | 12.666 | −51.232 | 25.177 | 1.00 | 12.67 |
| ATOM | 13838 | CA | GLU | 2052 | 13.198 | −52.051 | 26.267 | 1.00 | 13.06 |
| ATOM | 13839 | CB | GLU | 2052 | 12.177 | −52.152 | 27.405 | 1.00 | 17.19 |
| ATOM | 13840 | CG | GLU | 2052 | 12.650 | −52.976 | 28.597 | 1.00 | 22.09 |
| ATOM | 13841 | CD | GLU | 2052 | 11.791 | −52.760 | 29.836 | 1.00 | 27.02 |
| ATOM | 13842 | OE1 | GLU | 2052 | 11.779 | −53.647 | 30.714 | 1.00 | 29.74 |
| ATOM | 13843 | OE2 | GLU | 2052 | 11.135 | −51.702 | 29.940 | 1.00 | 27.52 |
| ATOM | 13844 | C | GLU | 2052 | 13.610 | −53.446 | 25.809 | 1.00 | 15.04 |
| ATOM | 13845 | O | GLU | 2052 | 14.600 | −54.006 | 26.299 | 1.00 | 12.91 |
| ATOM | 13846 | N | SER | 2053 | 12.866 | −54.005 | 24.862 | 1.00 | 14.34 |
| ATOM | 13847 | CA | SER | 2053 | 13.176 | −55.336 | 24.349 | 1.00 | 16.44 |
| ATOM | 13848 | CB | SER | 2053 | 11.939 | −55.945 | 23.689 | 1.00 | 17.92 |
| ATOM | 13849 | OG | SER | 2053 | 10.960 | −56.242 | 24.665 | 1.00 | 29.42 |
| ATOM | 13850 | C | SER | 2053 | 14.306 | −55.292 | 23.337 | 1.00 | 16.59 |
| ATOM | 13851 | O | SER | 2053 | 14.868 | −56.326 | 22.994 | 1.00 | 16.54 |
| ATOM | 13852 | N | GLY | 2054 | 14.632 | −54.096 | 22.856 | 1.00 | 14.35 |
| ATOM | 13853 | CA | GLY | 2054 | 15.695 | −53.961 | 21.877 | 1.00 | 14.92 |
| ATOM | 13854 | C | GLY | 2054 | 15.168 | −54.137 | 20.464 | 1.00 | 14.49 |
| ATOM | 13855 | O | GLY | 2054 | 15.930 | −54.058 | 19.503 | 1.00 | 14.84 |
| ATOM | 13856 | N | VAL | 2055 | 13.862 | −54.379 | 20.347 | 1.00 | 13.75 |
| ATOM | 13857 | CA | VAL | 2055 | 13.181 | −54.582 | 19.061 | 1.00 | 16.33 |
| ATOM | 13858 | CB | VAL | 2055 | 11.704 | −55.006 | 19.312 | 1.00 | 19.13 |
| ATOM | 13859 | CG1 | VAL | 2055 | 10.824 | −54.610 | 18.157 | 1.00 | 24.52 |
| ATOM | 13860 | CG2 | VAL | 2055 | 11.640 | −56.508 | 19.561 | 1.00 | 22.18 |
| ATOM | 13861 | C | VAL | 2055 | 13.203 | −53.333 | 18.181 | 1.00 | 15.78 |
| ATOM | 13862 | O | VAL | 2055 | 13.299 | −53.420 | 16.953 | 1.00 | 14.79 |
| ATOM | 13863 | N | TYR | 2056 | 13.077 | −52.177 | 18.823 | 1.00 | 13.64 |
| ATOM | 13864 | CA | TYR | 2056 | 13.105 | −50.895 | 18.121 | 1.00 | 14.23 |
| ATOM | 13865 | CB | TYR | 2056 | 11.816 | −50.108 | 18.336 | 1.00 | 14.11 |
| ATOM | 13866 | CG | TYR | 2056 | 11.878 | −48.760 | 17.647 | 1.00 | 13.46 |
| ATOM | 13867 | CD1 | TYR | 2056 | 11.680 | −48.653 | 16.270 | 1.00 | 14.58 |
| ATOM | 13868 | CE1 | TYR | 2056 | 11.857 | −47.443 | 15.605 | 1.00 | 13.65 |
| ATOM | 13869 | CD2 | TYR | 2056 | 12.248 | −47.614 | 18.349 | 1.00 | 11.87 |
| ATOM | 13870 | CE2 | TYR | 2056 | 12.430 | −46.397 | 17.696 | 1.00 | 13.86 |
| ATOM | 13871 | CZ | TYR | 2056 | 12.234 | −46.323 | 16.326 | 1.00 | 14.29 |
| ATOM | 13872 | OH | TYR | 2056 | 12.432 | −45.134 | 15.671 | 1.00 | 14.14 |
| ATOM | 13873 | C | TYR | 2056 | 14.266 | −50.086 | 18.691 | 1.00 | 13.20 |
| ATOM | 13874 | O | TYR | 2056 | 14.412 | −49.991 | 19.901 | 1.00 | 13.24 |
| ATOM | 13875 | N | PRO | 2057 | 15.092 | −49.477 | 17.825 | 1.00 | 15.12 |
| ATOM | 13876 | CD | PRO | 2057 | 16.137 | −48.516 | 18.218 | 1.00 | 16.15 |
| ATOM | 13877 | CA | PRO | 2057 | 14.952 | −49.547 | 16.371 | 1.00 | 15.54 |
| ATOM | 13878 | CB | PRO | 2057 | 15.799 | −48.374 | 15.880 | 1.00 | 18.49 |
| ATOM | 13879 | CG | PRO | 2057 | 16.865 | −43.270 | 16.917 | 1.00 | 17.17 |
| ATOM | 13880 | C | PRO | 2057 | 15.378 | −50.876 | 15.763 | 1.00 | 17.76 |
| ATOM | 13881 | O | PRO | 2057 | 16.236 | −51.580 | 16.301 | 1.00 | 16.91 |
| ATOM | 13882 | N | GLY | 2058 | 14.745 | −51.223 | 14.646 | 1.00 | 16.96 |
| ATOM | 13883 | CA | GLY | 2058 | 15.069 | −52.457 | 13.951 | 1.00 | 18.25 |
| ATOM | 13884 | C | GLY | 2058 | 16.151 | −52.181 | 12.932 | 1.00 | 19.23 |
| ATOM | 13885 | O | GLY | 2058 | 16.593 | −51.039 | 12.776 | 1.00 | 17.91 |
| ATOM | 13886 | N | GLU | 2059 | 16.590 | −53.220 | 12.234 | 1.00 | 18.48 |
| ATOM | 13887 | CA | GLU | 2059 | 17.627 | −53.048 | 11.225 | 1.00 | 19.82 |
| ATOM | 13888 | CB | GLU | 2059 | 17.963 | −54.396 | 10.572 | 1.00 | 22.37 |
| ATOM | 13889 | CG | GLU | 2059 | 19.024 | −54.287 | 9.491 | 1.00 | 25.19 |
| ATOM | 13890 | CD | GLU | 2059 | 20.374 | −53.864 | 10.050 | 1.00 | 26.27 |
| ATOM | 13891 | OE1 | GLU | 2059 | 21.173 | −53.288 | 9.285 | 1.00 | 28.76 |
| ATOM | 13892 | OE2 | GLU | 2059 | 20.637 | −54.117 | 11.246 | 1.00 | 24.77 |
| ATOM | 13893 | C | GLU | 2059 | 17.193 | −52.054 | 10.151 | 1.00 | 17.71 |
| ATOM | 13894 | O | GLU | 2059 | 18.007 | −51.273 | 9.657 | 1.00 | 16.86 |
| ATOM | 13895 | N | GLU | 2060 | 15.909 | −52.078 | 9.797 | 1.00 | 18.39 |
| ATOM | 13896 | CA | GLU | 2060 | 15.388 | −51.179 | 8.764 | 1.00 | 19.58 |
| ATOM | 13897 | CB | GLU | 2060 | 13.936 | −51.506 | 8.433 | 1.00 | 23.58 |
| ATOM | 13898 | CG | GLU | 2060 | 13.569 | −52.954 | 8.501 | 1.00 | 29.46 |
| ATOM | 13899 | CD | GLU | 2060 | 12.479 | −53.188 | 9.527 | 1.00 | 30.54 |
| ATOM | 13900 | OE1 | GLU | 2060 | 11.392 | −52.590 | 9.370 | 1.00 | 31.23 |
| ATOM | 13901 | OE2 | GLU | 2060 | 12.724 | −53.954 | 10.484 | 1.00 | 34.44 |
| ATOM | 13902 | C | GLU | 2060 | 15.443 | −49.723 | 9.197 | 1.00 | 18.24 |
| ATOM | 13903 | O | GLU | 2060 | 15.340 | −48.828 | 8.363 | 1.00 | 18.32 |
| ATOM | 13904 | N | HIS | 2061 | 15.592 | −49.495 | 10.499 | 1.00 | 17.85 |
| ATOM | 13905 | CA | HIS | 2061 | 15.646 | −48.138 | 11.050 | 1.00 | 15.77 |
| ATOM | 13906 | CB | HIS | 2061 | 14.890 | −48.075 | 12.374 | 1.00 | 15.70 |
| ATOM | 13907 | CG | HIS | 2061 | 13.485 | −48.573 | 12.296 | 1.00 | 13.96 |
| ATOM | 13908 | CD2 | HIS | 2061 | 12.870 | −49.609 | 12.912 | 1.00 | 11.61 |
| ATOM | 13909 | ND1 | HIS | 2061 | 12.526 | −47.964 | 11.518 | 1.00 | 14.67 |
| ATOM | 13910 | CE1 | HIS | 2061 | 11.378 | −48.601 | 11.660 | 1.00 | 12.03 |
| ATOM | 13911 | NE2 | HIS | 2061 | 11.559 | −49.603 | 12.500 | 1.00 | 17.48 |
| ATOM | 13912 | C | HIS | 2061 | 17.074 | −47.702 | 11.324 | 1.00 | 16.96 |
| ATOM | 13913 | O | HIS | 2061 | 17.292 | −46.639 | 11.899 | 1.00 | 16.77 |
| ATOM | 13914 | N | SER | 2062 | 18.040 | −48.516 | 10.913 | 1.00 | 17.85 |
| ATOM | 13915 | CA | SER | 2062 | 19.449 | −48.230 | 11.175 | 1.00 | 18.95 |
| ATOM | 13916 | CB | SER | 2062 | 20.056 | −49.420 | 11.926 | 1.00 | 18.81 |
| ATOM | 13917 | OG | SER | 2062 | 19.261 | −49.760 | 13.059 | 1.00 | 21.12 |
| ATOM | 13918 | C | SER | 2062 | 20.298 | −47.917 | 9.941 | 1.00 | 21.62 |
| ATOM | 13919 | O | SER | 2062 | 19.945 | −48.283 | 8.813 | 1.00 | 21.18 |
| ATOM | 13920 | N | PHE | 2063 | 21.423 | −47.240 | 10.170 | 1.00 | 22.99 |
| ATOM | 13921 | CA | PHE | 2063 | 22.359 | −46.870 | 9.099 | 1.00 | 26.28 |
| ATOM | 13922 | CB | PHE | 2063 | 22.600 | −45.348 | 9.075 | 1.00 | 26.42 |
| ATOM | 13923 | CG | PHE | 2063 | 21.360 | −44.526 | 8.820 | 1.00 | 29.55 |
| ATOM | 13924 | CD1 | PHE | 2063 | 20.806 | −43.746 | 9.835 | 1.00 | 31.06 |
| ATOM | 13925 | CD2 | PHE | 2063 | 20.754 | −44.519 | 7.568 | 1.00 | 29.76 |
| ATOM | 13926 | CE1 | PHE | 2063 | 19.663 | −42.974 | 9.607 | 1.00 | 30.50 |
| ATOM | 13927 | CE2 | PHE | 2063 | 19.610 | −43.750 | 7.328 | 1.00 | 31.05 |
| ATOM | 13928 | CZ | PHE | 2063 | 19.065 | −42.976 | 8.350 | 1.00 | 29.87 |
| ATOM | 13929 | C | PHE | 2063 | 23.701 | −47.577 | 9.301 | 1.00 | 26.42 |
| ATOM | 13930 | O | PHE | 2063 | 24.107 | −47.849 | 10.432 | 1.00 | 22.96 |
| ATOM | 13931 | N | HIS | 2064 | 24.386 | −47.867 | 8.199 | 1.00 | 28.64 |
| ATOM | 13932 | CA | HIS | 2064 | 25.687 | −48.543 | 8.246 | 1.00 | 31.07 |
| ATOM | 13933 | CB | HIS | 2064 | 25.543 | −49.996 | 7.789 | 1.00 | 31.23 |
| ATOM | 13934 | CG | HIS | 2064 | 24.784 | −50.855 | 8.749 | 1.00 | 31.06 |
| ATOM | 13935 | CD2 | HIS | 2064 | 23.523 | −51.345 | 8.704 | 1.00 | 30.91 |
| ATOM | 13936 | ND1 | HIS | 2064 | 25.320 | −51.287 | 9.943 | 1.00 | 32.43 |
| ATOM | 13937 | CE1 | HIS | 2064 | 24.423 | −52.007 | 10.593 | 1.00 | 32.22 |
| ATOM | 13938 | NE2 | HIS | 2064 | 23.323 | −52.058 | 9.863 | 1.00 | 32.35 |
| ATOM | 13939 | C | HIS | 2064 | 26.729 | −47.836 | 7.381 | 1.00 | 31.64 |
| ATOM | 13940 | O | HIS | 2064 | 27.892 | −47.757 | 7.818 | 1.00 | 32.53 |
| ATOM | 13941 | OXT | HIS | 2064 | 26.375 | −47.385 | 6.272 | 1.00 | 34.83 |
| ATOM | 13942 | C1 | KPL | 2065 | 15.474 | −35.267 | 17.263 | 1.00 | 36.48 |
| ATOM | 13943 | C2 | KPL | 2065 | 16.103 | −34.899 | 18.622 | 1.00 | 35.77 |
| ATOM | 13944 | C3 | KPL | 2065 | 15.239 | −35.486 | 19.739 | 1.00 | 36.51 |
| ATOM | 13945 | C4 | KPL | 2065 | 17.519 | −35.515 | 18.719 | 1.00 | 37.71 |
| ATOM | 13946 | O1 | KPL | 2065 | 18.376 | −35.011 | 17.682 | 1.00 | 42.74 |
| ATOM | 13947 | C5 | KPL | 2065 | 16.164 | −33.356 | 18.773 | 1.00 | 32.73 |
| ATOM | 13948 | O2 | KPL | 2065 | 17.230 | −32.800 | 18.938 | 1.00 | 31.75 |
| ATOM | 13949 | C6 | KPL | 2065 | 14.923 | −32.498 | 18.721 | 1.00 | 31.70 |
| ATOM | 13950 | O3 | KPL | 2065 | 13.821 | −33.000 | 18.568 | 1.00 | 31.03 |
| ATOM | 13951 | O4 | KPL | 2065 | 15.041 | −31.157 | 18.845 | 1.00 | 20.56 |
| ATOM | 13952 | CB | MET | 2101 | 22.414 | −8.383 | 70.247 | 1.00 | 66.53 |
| ATOM | 13953 | CG | MET | 2101 | 22.617 | −8.021 | 71.717 | 1.00 | 69.29 |
| ATOM | 13954 | SD | MET | 2101 | 22.027 | −6.373 | 72.186 | 1.00 | 72.42 |
| ATOM | 13955 | CE | MET | 2101 | 23.584 | −5.466 | 72.275 | 1.00 | 72.02 |
| ATOM | 13956 | C | MET | 2101 | 22.338 | −6.232 | 68.953 | 1.00 | 62.58 |
| ATOM | 13957 | O | MET | 2101 | 21.157 | −6.314 | 68.610 | 1.00 | 62.34 |
| ATOM | 13958 | N | MET | 2101 | 24.527 | −7.178 | 69.751 | 1.00 | 64.37 |
| ATOM | 13959 | CA | MET | 2101 | 23.158 | −7.490 | 69.244 | 1.00 | 64.28 |
| ATOM | 13960 | N | LYS | 2102 | 22.975 | −5.073 | 69.085 | 1.00 | 60.08 |
| ATOM | 13961 | CA | LYS | 2102 | 22.317 | −3.798 | 68.834 | 1.00 | 57.36 |
| ATOM | 13962 | CB | LYS | 2102 | 22.010 | −3.086 | 70.157 | 1.00 | 58.35 |
| ATOM | 13963 | CG | LYS | 2102 | 20.762 | −3.591 | 70.868 | 1.00 | 60.33 |
| ATOM | 13964 | CD | LYS | 2102 | 19.496 | −3.202 | 70.117 | 1.00 | 61.31 |
| ATOM | 13965 | CE | LYS | 2102 | 19.296 | −1.693 | 70.111 | 1.00 | 62.35 |
| ATOM | 13966 | NZ | LYS | 2102 | 18.073 | −1.296 | 69.354 | 1.00 | 63.75 |
| ATOM | 13967 | C | LYS | 2102 | 23.179 | −2.896 | 67.957 | 1.00 | 54.18 |
| ATOM | 13968 | O | LYS | 2102 | 23.678 | −1.865 | 68.413 | 1.00 | 54.59 |
| ATOM | 13969 | N | PRO | 2103 | 23.371 | −3.276 | 66.685 | 1.00 | 50.17 |
| ATOM | 13970 | CD | PRO | 2103 | 23.878 | −2.346 | 65.660 | 1.00 | 49.96 |
| ATOM | 13971 | CA | PRO | 2103 | 22.830 | −4.484 | 66.053 | 1.00 | 46.64 |
| ATOM | 13972 | CB | PRO | 2103 | 22.605 | −4.032 | 64.620 | 1.00 | 47.38 |
| ATOM | 13973 | CG | PRO | 2103 | 23.802 | −3.176 | 64.386 | 1.00 | 48.98 |
| ATOM | 13974 | C | PRO | 2103 | 23.794 | −5.673 | 66.131 | 1.00 | 42.70 |
| ATOM | 13975 | O | PRO | 2103 | 24.924 | −5.536 | 66.596 | 1.00 | 41.32 |
| ATOM | 13976 | N | THR | 2104 | 23.334 | −6.832 | 65.667 | 1.00 | 38.31 |
| ATOM | 13977 | CA | THR | 2104 | 24.144 | −8.045 | 65.662 | 1.00 | 35.00 |
| ATOM | 13978 | CB | THR | 2104 | 23.259 | −9.309 | 65.701 | 1.00 | 35.04 |
| ATOM | 13979 | OG1 | THR | 2104 | 22.442 | −9.286 | 66.875 | 1.00 | 34.66 |
| ATOM | 13980 | CG2 | THR | 2104 | 24.116 | −10.561 | 65.714 | 1.00 | 34.54 |
| ATOM | 13981 | C | THR | 2104 | 24.981 | −8.080 | 64.384 | 1.00 | 33.55 |
| ATOM | 13982 | O | THR | 2104 | 24.455 | −7.872 | 63.291 | 1.00 | 31.34 |
| ATOM | 13983 | N | THR | 2105 | 26.279 | −8.340 | 64.521 | 1.00 | 31.77 |
| ATOM | 13984 | CA | THR | 2105 | 27.172 | −8.391 | 63.365 | 1.00 | 30.32 |
| ATOM | 13985 | CB | THR | 2105 | 28.127 | −7.175 | 63.336 | 1.00 | 30.72 |
| ATOM | 13986 | OG1 | THR | 2105 | 28.999 | −7.223 | 64.471 | 1.00 | 32.36 |
| ATOM | 13987 | CG2 | THR | 2105 | 27.338 | −5.874 | 63.366 | 1.00 | 29.57 |
| ATOM | 13988 | C | THR | 2105 | 28.013 | −9.664 | 63.355 | 1.00 | 29.36 |
| ATOM | 13989 | O | THR | 2105 | 27.945 | −10.474 | 64.281 | 1.00 | 29.05 |
| ATOM | 13990 | N | ILE | 2106 | 28.798 | −9.837 | 62.297 | 1.00 | 28.45 |
| ATOM | 13991 | CA | ILE | 2106 | 29.666 | −11.004 | 62.162 | 1.00 | 28.68 |
| ATOM | 13992 | CB | ILE | 2106 | 30.529 | −10.912 | 60.893 | 1.00 | 28.87 |
| ATOM | 13993 | CG2 | ILE | 2106 | 31.287 | −12.221 | 60.690 | 1.00 | 28.27 |
| ATOM | 13994 | CG1 | ILE | 2106 | 29.639 | −10.618 | 59.681 | 1.00 | 31.64 |
| ATOM | 13995 | CD1 | ILE | 2106 | 30.409 | −10.317 | 58.403 | 1.00 | 31.18 |
| ATOM | 13996 | C | ILE | 2106 | 30.599 | −11.079 | 63.364 | 1.00 | 29.36 |
| ATOM | 13997 | O | ILE | 2106 | 30.944 | −12.164 | 63.833 | 1.00 | 29.25 |
| ATOM | 13998 | N | SER | 2107 | 31.003 | −9.912 | 63.854 | 1.00 | 29.55 |
| ATOM | 13999 | CA | SER | 2107 | 31.897 | −9.820 | 65.002 | 1.00 | 30.81 |
| ATOM | 14000 | CB | SER | 2107 | 32.058 | −8.358 | 65.425 | 1.00 | 31.74 |
| ATOM | 14001 | OG | SER | 2107 | 32.548 | −7.568 | 64.361 | 1.00 | 34.72 |
| ATOM | 14002 | C | SER | 2107 | 31.363 | −10.632 | 66.175 | 1.00 | 29.75 |
| ATOM | 14003 | O | SER | 2107 | 32.131 | −11.245 | 66.904 | 1.00 | 29.24 |
| ATOM | 14004 | N | LEU | 2108 | 30.045 | −10.630 | 66.348 | 1.00 | 29.42 |
| ATOM | 14005 | CA | LEU | 2108 | 29.419 | −11.368 | 67.436 | 1.00 | 29.77 |
| ATOM | 14006 | CB | LEU | 2108 | 27.925 | −11.045 | 67.512 | 1.00 | 30.86 |
| ATOM | 14007 | CG | LEU | 2108 | 27.356 | −10.598 | 68.865 | 1.00 | 32.78 |
| ATOM | 14008 | CD1 | LEU | 2108 | 25.830 | −10.637 | 68.802 | 1.00 | 33.38 |
| ATOM | 14009 | CD2 | LEU | 2108 | 27.851 | −11.506 | 69.983 | 1.00 | 32.44 |
| ATOM | 14010 | C | LEU | 2108 | 29.601 | −12.874 | 67.267 | 1.00 | 29.98 |
| ATOM | 14011 | O | LEU | 2108 | 29.949 | −13.575 | 68.216 | 1.00 | 28.71 |
| ATOM | 14012 | N | LEU | 2109 | 29.366 | −13.369 | 66.055 | 1.00 | 28.45 |
| ATOM | 14013 | CA | LEU | 2109 | 29.504 | −14.795 | 65.783 | 1.00 | 28.96 |
| ATOM | 14014 | CB | LEU | 2109 | 29.031 | −15.112 | 64.360 | 1.00 | 26.96 |
| ATOM | 14015 | CG | LEU | 2109 | 27.608 | −14.653 | 63.997 | 1.00 | 26.16 |
| ATOM | 14016 | CD1 | LEU | 2109 | 27.228 | −15.201 | 62.628 | 1.00 | 24.61 |
| ATOM | 14017 | CD2 | LEU | 2109 | 26.621 | −15.140 | 65.046 | 1.00 | 23.92 |
| ATOM | 14018 | C | LEU | 2109 | 30.955 | −15.232 | 65.962 | 1.00 | 30.20 |
| ATOM | 14019 | O | LEU | 2109 | 31.234 | −16.329 | 66.455 | 1.00 | 27.70 |
| ATOM | 14020 | N | GLN | 2110 | 31.879 | −14.366 | 65.563 | 1.00 | 32.67 |
| ATOM | 14021 | CA | GLN | 2110 | 33.299 | −14.666 | 65.691 | 1.00 | 35.69 |
| ATOM | 14022 | CB | GLN | 2110 | 34.131 | −13.556 | 65.041 | 1.00 | 37.71 |
| ATOM | 14023 | CG | GLN | 2110 | 35.596 | −13.909 | 64.805 | 1.00 | 40.40 |
| ATOM | 14024 | CD | GLN | 2110 | 35.777 | −15.115 | 63.894 | 1.00 | 41.71 |
| ATOM | 14025 | OE1 | GLN | 2110 | 35.575 | −16.259 | 64.303 | 1.00 | 42.12 |
| ATOM | 14026 | NE2 | GLN | 2110 | 36.156 | −14.858 | 62.645 | 1.00 | 42.57 |
| ATOM | 14027 | C | GLN | 2110 | 33.619 | −14.778 | 67.179 | 1.00 | 37.51 |
| ATOM | 14028 | O | GLN | 2110 | 34.441 | −15.595 | 67.588 | 1.00 | 39.31 |
| ATOM | 14029 | N | LYS | 2111 | 32.954 | −13.956 | 67.987 | 1.00 | 38.46 |
| ATOM | 14030 | CA | LYS | 2111 | 33.157 | −13.980 | 69.430 | 1.00 | 39.03 |
| ATOM | 14031 | CB | LYS | 2111 | 32.407 | −12.824 | 70.098 | 1.00 | 41.13 |
| ATOM | 14032 | CG | LYS | 2111 | 32.493 | −12.839 | 71.621 | 1.00 | 44.09 |
| ATOM | 14033 | CD | LYS | 2111 | 31.376 | −12.035 | 72.275 | 1.00 | 45.16 |
| ATOM | 14034 | CE | LYS | 2111 | 31.450 | −10.557 | 71.933 | 1.00 | 46.74 |
| ATOM | 14035 | NZ | LYS | 2111 | 30.408 | −9.785 | 72.674 | 1.00 | 47.25 |
| ATOM | 14036 | C | LYS | 2111 | 32.637 | −15.301 | 69.989 | 1.00 | 38.63 |
| ATOM | 14037 | O | LYS | 2111 | 33.282 | −15.931 | 70.825 | 1.00 | 37.38 |
| ATOM | 14038 | N | TYR | 2112 | 31.463 | −15.714 | 69.521 | 1.00 | 38.15 |
| ATOM | 14039 | CA | TYR | 2112 | 30.845 | −16.959 | 69.970 | 1.00 | 37.96 |
| ATOM | 14040 | CB | TYR | 2112 | 29.506 | −17.168 | 69.256 | 1.00 | 39.49 |
| ATOM | 14041 | CG | TYR | 2112 | 28.385 | −16.279 | 69.755 | 1.00 | 41.55 |
| ATOM | 14042 | CD1 | TYR | 2112 | 27.152 | −16.250 | 69.101 | 1.00 | 42.17 |
| ATOM | 14043 | CE1 | TYR | 2112 | 26.106 | −15.461 | 69.569 | 1.00 | 43.58 |
| ATOM | 14044 | CD2 | TYR | 2112 | 28.544 | −15.488 | 70.897 | 1.00 | 42.83 |
| ATOM | 14045 | CE2 | TYR | 2112 | 27.503 | −14.695 | 71.374 | 1.00 | 43.95 |
| ATOM | 14046 | CZ | TYR | 2112 | 26.288 | −14.687 | 70.707 | 1.00 | 44.41 |
| ATOM | 14047 | OH | TYR | 2112 | 25.256 | −13.912 | 71.176 | 1.00 | 46.08 |
| ATOM | 14048 | C | TYR | 2112 | 31.725 | −18.190 | 69.761 | 1.00 | 37.30 |
| ATOM | 14049 | O | TYR | 2112 | 31.765 | −19.080 | 70.610 | 1.00 | 35.99 |
| ATOM | 14050 | N | LYS | 2113 | 32.417 | −18.254 | 68.628 | 1.00 | 36.90 |
| ATOM | 14051 | CA | LYS | 2113 | 33.280 | −19.400 | 68.362 | 1.00 | 38.21 |
| ATOM | 14052 | CB | LYS | 2113 | 33.784 | −19.378 | 66.914 | 1.00 | 36.95 |
| ATOM | 14053 | CG | LYS | 2113 | 34.652 | −20.579 | 66.560 | 1.00 | 34.27 |
| ATOM | 14054 | CD | LYS | 2113 | 34.828 | −20.750 | 65.061 | 1.00 | 32.97 |
| ATOM | 14055 | CE | LYS | 2113 | 35.585 | −22.041 | 64.761 | 1.00 | 32.84 |
| ATOM | 14056 | NZ | LYS | 2113 | 35.432 | −22.499 | 63.348 | 1.00 | 30.32 |
| ATOM | 14057 | C | LYS | 2113 | 34.465 | −19.411 | 69.328 | 1.00 | 38.99 |
| ATOM | 14058 | O | LYS | 2113 | 34.891 | −20.471 | 69.782 | 1.00 | 38.16 |
| ATOM | 14059 | N | GLN | 2114 | 34.988 | −18.229 | 69.639 | 1.00 | 40.94 |
| ATOM | 14060 | CA | GLN | 2114 | 36.116 | −18.110 | 70.557 | 1.00 | 42.72 |
| ATOM | 14061 | CB | GLN | 2114 | 36.574 | −16.653 | 70.652 | 1.00 | 44.16 |
| ATOM | 14062 | CG | GLN | 2114 | 37.245 | −16.133 | 69.390 | 1.00 | 48.37 |
| ATOM | 14063 | CD | GLN | 2114 | 37.620 | −14.663 | 69.486 | 1.00 | 51.00 |
| ATOM | 14064 | OE1 | GLN | 2114 | 38.357 | −14.254 | 70.386 | 1.00 | 53.01 |
| ATOM | 14065 | NE2 | GLN | 2114 | 37.117 | −13.861 | 68.551 | 1.00 | 51.71 |
| ATOM | 14066 | C | GLN | 2114 | 35.736 | −18.617 | 71.942 | 1.00 | 42.63 |
| ATOM | 14067 | O | GLN | 2114 | 36.537 | −19.263 | 72.616 | 1.00 | 42.99 |
| ATOM | 14068 | N | GLU | 2115 | 34.508 | −18.320 | 72.358 | 1.00 | 42.51 |
| ATOM | 14069 | CA | GLU | 2115 | 34.016 | −18.745 | 73.664 | 1.00 | 42.44 |
| ATOM | 14070 | CB | GLU | 2115 | 33.001 | −17.736 | 74.207 | 1.00 | 44.97 |
| ATOM | 14071 | CG | GLU | 2115 | 33.467 | −16.292 | 74.162 | 1.00 | 47.71 |
| ATOM | 14072 | CD | GLU | 2115 | 32.482 | −15.348 | 74.816 | 1.00 | 49.87 |
| ATOM | 14073 | OE1 | GLU | 2115 | 31.281 | −15.405 | 74.472 | 1.00 | 50.50 |
| ATOM | 14074 | OE2 | GLU | 2115 | 32.911 | −14.545 | 75.674 | 1.00 | 52.16 |
| ATOM | 14075 | C | GLU | 2115 | 33.356 | −20.114 | 73.558 | 1.00 | 40.83 |
| ATOM | 14076 | O | GLU | 2115 | 32.806 | −20.624 | 74.532 | 1.00 | 40.15 |
| ATOM | 14077 | N | LYS | 2116 | 33.410 | −20.701 | 72.368 | 1.00 | 39.34 |
| ATOM | 14078 | CA | LYS | 2116 | 32.819 | −22.010 | 72.130 | 1.00 | 38.09 |
| ATOM | 14079 | CB | LYS | 2116 | 33.578 | −23.079 | 72.923 | 1.00 | 40.37 |
| ATOM | 14080 | CG | LYS | 2116 | 35.049 | −23.210 | 72.540 | 1.00 | 42.96 |
| ATOM | 14081 | CD | LYS | 2116 | 35.213 | −23.602 | 71.075 | 1.00 | 45.90 |
| ATOM | 14082 | CE | LYS | 2116 | 36.679 | −23.631 | 70.646 | 1.00 | 47.03 |
| ATOM | 14083 | NZ | LYS | 2116 | 37.471 | −24.671 | 71.351 | 1.00 | 46.52 |
| ATOM | 14084 | C | LYS | 2116 | 31.332 | −22.042 | 72.493 | 1.00 | 36.61 |
| ATOM | 14085 | O | LYS | 2116 | 30.836 | −23.016 | 73.062 | 1.00 | 35.86 |
| ATOM | 14086 | N | LYS | 2117 | 30.620 | −20.972 | 72.159 | 1.00 | 35.40 |
| ATOM | 14087 | CA | LYS | 2117 | 29.193 | −20.908 | 72.444 | 1.00 | 33.94 |
| ATOM | 14088 | CB | LYS | 2117 | 28.838 | −19.570 | 73.100 | 1.00 | 34.82 |
| ATOM | 14089 | CG | LYS | 2117 | 27.338 | −19.354 | 73.271 | 1.00 | 36.44 |
| ATOM | 14090 | CD | LYS | 2117 | 27.039 | −18.265 | 74.290 | 1.00 | 38.88 |
| ATOM | 14091 | CE | LYS | 2117 | 27.406 | −18.729 | 75.695 | 1.00 | 39.51 |
| ATOM | 14092 | NZ | LYS | 2117 | 27.253 | −17.658 | 76.714 | 1.00 | 41.13 |
| ATOM | 14093 | C | LYS | 2117 | 28.381 | −21.107 | 71.166 | 1.00 | 32.80 |
| ATOM | 14094 | O | LYS | 2117 | 28.336 | −20.232 | 70.300 | 1.00 | 31.72 |
| ATOM | 14095 | N | ARG | 2118 | 27.752 | −22.274 | 71.060 | 1.00 | 30.34 |
| ATOM | 14096 | CA | ARG | 2118 | 26.938 | −22.628 | 69.902 | 1.00 | 30.20 |
| ATOM | 14097 | CB | ARG | 2118 | 26.539 | −24.105 | 69.984 | 1.00 | 30.53 |
| ATOM | 14098 | CG | ARG | 2118 | 27.675 | −25.045 | 69.596 | 1.00 | 32.66 |
| ATOM | 14099 | CD | ARG | 2118 | 27.360 | −26.506 | 69.872 | 1.00 | 33.98 |
| ATOM | 14100 | NE | ARG | 2118 | 27.573 | −26.853 | 71.274 | 1.00 | 34.15 |
| ATOM | 14101 | CZ | ARG | 2118 | 27.601 | −28.097 | 71.741 | 1.00 | 33.23 |
| ATOM | 14102 | NH1 | ARG | 2118 | 27.427 | −29.121 | 70.918 | 1.00 | 33.99 |
| ATOM | 14103 | NH2 | ARG | 2118 | 27.816 | −28.321 | 73.031 | 1.00 | 31.99 |
| ATOM | 14104 | C | ARG | 2118 | 25.703 | −21.734 | 69.800 | 1.00 | 28.50 |
| ATOM | 14105 | O | ARG | 2118 | 24.919 | −21.635 | 70.741 | 1.00 | 28.26 |
| ATOM | 14106 | N | PHE | 2119 | 25.546 | −21.089 | 68.647 | 1.00 | 27.02 |
| ATOM | 14107 | CA | PHE | 2119 | 24.440 | −20.171 | 68.403 | 1.00 | 25.78 |
| ATOM | 14108 | CB | PHE | 2119 | 25.002 | −18.816 | 67.965 | 1.00 | 26.66 |
| ATOM | 14109 | CG | PHE | 2119 | 25.831 | −18.883 | 66.710 | 1.00 | 28.65 |
| ATOM | 14110 | CD1 | PHE | 2119 | 25.236 | −18.742 | 65.457 | 1.00 | 27.88 |
| ATOM | 14111 | CD2 | PHE | 2119 | 27.205 | −19.110 | 66.779 | 1.00 | 27.56 |
| ATOM | 14112 | CE1 | PHE | 2119 | 25.997 | −18.821 | 64.294 | 1.00 | 29.01 |
| ATOM | 14113 | CE2 | PHE | 2119 | 27.975 | −19.192 | 65.624 | 1.00 | 28.48 |
| ATOM | 14114 | CZ | PHE | 2119 | 27.371 | −19.047 | 64.377 | 1.00 | 29.61 |
| ATOM | 14115 | C | PHE | 2119 | 23.464 | −20.693 | 67.351 | 1.00 | 24.24 |
| ATOM | 14116 | O | PHE | 2119 | 23.861 | −21.375 | 66.404 | 1.00 | 20.96 |
| ATOM | 14117 | N | ALA | 2120 | 22.186 | −20.365 | 67.515 | 1.00 | 23.46 |
| ATOM | 14118 | CA | ALA | 2120 | 21.162 | −20.810 | 66.577 | 1.00 | 23.11 |
| ATOM | 14119 | CB | ALA | 2120 | 19.886 | −21.175 | 67.343 | 1.00 | 25.46 |
| ATOM | 14120 | C | ALA | 2120 | 20.857 | −19.756 | 65.510 | 1.00 | 22.49 |
| ATOM | 14121 | O | ALA | 2120 | 20.928 | −18.553 | 65.765 | 1.00 | 20.69 |
| ATOM | 14122 | N | THR | 2121 | 20.529 | −20.225 | 64.309 | 1.00 | 20.02 |
| ATOM | 14123 | CA | THR | 2121 | 20.198 | −19.350 | 63.186 | 1.00 | 21.26 |
| ATOM | 14124 | CB | THR | 2121 | 21.324 | −19.357 | 62.125 | 1.00 | 22.14 |
| ATOM | 14125 | OG1 | THR | 2121 | 22.552 | −18.930 | 62.734 | 1.00 | 27.92 |
| ATOM | 14126 | CG2 | THR | 2121 | 20.987 | −18.424 | 60.985 | 1.00 | 29.91 |
| ATOM | 14127 | C | THR | 2121 | 18.923 | −19.922 | 62.577 | 1.00 | 17.16 |
| ATOM | 14128 | O | THR | 2121 | 18.651 | −21.104 | 62.731 | 1.00 | 17.14 |
| ATOM | 14129 | N | ILE | 2122 | 18.139 | −19.102 | 61.885 | 1.00 | 18.16 |
| ATOM | 14130 | CA | ILE | 2122 | 16.898 | −19.608 | 61.308 | 1.00 | 16.98 |
| ATOM | 14131 | CB | ILE | 2122 | 15.737 | −19.454 | 62.321 | 1.00 | 19.66 |
| ATOM | 14132 | CG2 | ILE | 2122 | 15.365 | −17.977 | 62.470 | 1.00 | 18.99 |
| ATOM | 14133 | CG1 | ILE | 2122 | 14.526 | −20.260 | 61.856 | 1.00 | 20.97 |
| ATOM | 14134 | CD1 | ILE | 2122 | 13.404 | −20.310 | 62.873 | 1.00 | 23.87 |
| ATOM | 14135 | C | ILE | 2122 | 16.545 | −18.882 | 60.020 | 1.00 | 16.60 |
| ATOM | 14136 | O | ILE | 2122 | 17.017 | −17.772 | 59.792 | 1.00 | 17.43 |
| ATOM | 14137 | N | THR | 2123 | 15.743 | −19.513 | 59.166 | 1.00 | 18.07 |
| ATOM | 14138 | CA | THR | 2123 | 15.330 | −18.866 | 57.923 | 1.00 | 17.40 |
| ATOM | 14139 | CB | THR | 2123 | 14.902 | −19.892 | 56.836 | 1.00 | 19.53 |
| ATOM | 14140 | OG1 | THR | 2123 | 13.739 | −20.612 | 57.278 | 1.00 | 20.71 |
| ATOM | 14141 | CG2 | THR | 2123 | 16.039 | −20.876 | 56.547 | 1.00 | 19.80 |
| ATOM | 14142 | C | THR | 2123 | 14.134 | −17.973 | 58.229 | 1.00 | 16.12 |
| ATOM | 14143 | O | THR | 2123 | 13.373 | −18.244 | 59.152 | 1.00 | 17.78 |
| ATOM | 14144 | N | ALA | 2124 | 13.978 | −16.903 | 57.459 | 1.00 | 16.44 |
| ATOM | 14145 | CA | ALA | 2124 | 12.863 | −15.976 | 57.646 | 1.00 | 15.59 |
| ATOM | 14146 | CB | ALA | 2124 | 13.226 | −14.918 | 58.685 | 1.00 | 17.03 |
| ATOM | 14147 | C | ALA | 2124 | 12.600 | −15.330 | 56.291 | 1.00 | 14.67 |
| ATOM | 14148 | O | ALA | 2124 | 13.539 | −15.046 | 55.556 | 1.00 | 13.64 |
| ATOM | 14149 | N | TYR | 2125 | 11.331 | −15.107 | 55.956 | 1.00 | 13.33 |
| ATOM | 14150 | CA | TYR | 2125 | 10.985 | −14.521 | 54.659 | 1.00 | 12.55 |
| ATOM | 14151 | CB | TYR | 2125 | 10.439 | −15.599 | 53.712 | 1.00 | 14.08 |
| ATOM | 14152 | CG | TYR | 2125 | 11.166 | −16.926 | 53.797 | 1.00 | 13.16 |
| ATOM | 14153 | CD1 | TYR | 2125 | 10.597 | −18.007 | 54.464 | 1.00 | 15.20 |
| ATOM | 14154 | CE2 | TYR | 2125 | 11.268 | −19.220 | 54.578 | 1.00 | 19.44 |
| ATOM | 14155 | CD2 | TYR | 2125 | 12.436 | −17.090 | 53.240 | 1.00 | 15.12 |
| ATOM | 14156 | CE2 | TYR | 2125 | 13.122 | −18.308 | 53.351 | 1.00 | 16.49 |
| ATOM | 14157 | CZ | TYR | 2125 | 12.527 | −19.362 | 54.023 | 1.00 | 17.45 |
| ATOM | 14158 | OH | TYR | 2125 | 13.190 | −20.559 | 54.160 | 1.00 | 18.95 |
| ATOM | 14159 | C | TYR | 2125 | 9.957 | −13.401 | 54.752 | 1.00 | 14.84 |
| ATOM | 14160 | O | TYR | 2125 | 9.513 | −12.876 | 53.726 | 1.00 | 13.18 |
| ATOM | 14161 | N | ASP | 2126 | 9.569 | −13.030 | 55.967 | 1.00 | 15.18 |
| ATOM | 14162 | CA | ASP | 2126 | 8.586 | −11.969 | 56.114 | 1.00 | 15.35 |
| ATOM | 14163 | CB | ASP | 2126 | 7.164 | −12.552 | 55.980 | 1.00 | 16.11 |
| ATOM | 14164 | CG | ASP | 2126 | 6.804 | −13.517 | 57.105 | 1.00 | 17.38 |
| ATOM | 14165 | OD1 | ASP | 2126 | 6.660 | −13.064 | 58.256 | 1.00 | 17.19 |
| ATOM | 14166 | OD2 | ASP | 2126 | 6.665 | −14.729 | 56.837 | 1.00 | 17.99 |
| ATOM | 14167 | C | ASP | 2126 | 8.740 | −11.166 | 57.410 | 1.00 | 16.68 |
| ATOM | 14168 | O | ASP | 2126 | 9.480 | −11.549 | 58.325 | 1.00 | 15.28 |
| ATOM | 14169 | N | TYR | 2127 | 8.046 | −10.038 | 57.475 | 1.00 | 15.28 |
| ATOM | 14170 | CA | TYR | 2127 | 8.120 | −9.164 | 58.641 | 1.00 | 17.17 |
| ATOM | 14171 | CB | TYR | 2127 | 7.276 | −7.913 | 58.397 | 1.00 | 17.79 |
| ATOM | 14172 | CG | TYR | 2127 | 6.992 | −7.109 | 59.641 | 1.00 | 17.73 |
| ATOM | 14173 | CD1 | TYR | 2127 | 7.875 | −6.122 | 60.075 | 1.00 | 20.38 |
| ATOM | 14174 | CE1 | TYR | 2127 | 7.605 | −5.367 | 61.218 | 1.00 | 20.48 |
| ATOM | 14175 | CD2 | TYR | 2127 | 5.832 | −7.331 | 60.379 | 1.00 | 20.09 |
| ATOM | 14176 | CE2 | TYR | 2127 | 5.551 | −6.590 | 61.519 | 1.00 | 22.06 |
| ATOM | 14177 | CZ | TYR | 2127 | 6.439 | −5.610 | 61.930 | 1.00 | 23.04 |
| ATOM | 14178 | OH | TYR | 2127 | 6.152 | −4.857 | 63.050 | 1.00 | 24.49 |
| ATOM | 14179 | C | TYR | 2127 | 7.671 | −9.827 | 59.937 | 1.00 | 18.30 |
| ATOM | 14180 | O | TYR | 2127 | 8.354 | −9.732 | 60.961 | 1.00 | 17.92 |
| ATOM | 14181 | N | SER | 2128 | 6.518 | −10.487 | 59.886 | 1.00 | 19.07 |
| ATOM | 14182 | CA | SER | 2128 | 5.943 | −11.136 | 61.055 | 1.00 | 21.09 |
| ATOM | 14183 | CB | SER | 2128 | 4.638 | −11.836 | 60.682 | 1.00 | 22.18 |
| ATOM | 14184 | OG | SER | 2128 | 3.632 | −10.872 | 60.424 | 1.00 | 21.45 |
| ATOM | 14185 | C | SER | 2128 | 6.861 | −12.120 | 61.754 | 1.00 | 21.26 |
| ATOM | 14186 | O | SER | 2128 | 7.154 | −11.960 | 62.943 | 1.00 | 20.52 |
| ATOM | 14187 | N | PHE | 2129 | 7.310 | −13.143 | 61.036 | 1.00 | 18.91 |
| ATOM | 14188 | CA | PHE | 2129 | 8.194 | −14.111 | 61.661 | 1.00 | 18.72 |
| ATOM | 14189 | CB | PHE | 2129 | 8.404 | −15.338 | 60.770 | 1.00 | 19.49 |
| ATOM | 14190 | CG | PHE | 2129 | 7.255 | −16.303 | 60.797 | 1.00 | 20.01 |
| ATOM | 14191 | CD1 | PHE | 2129 | 6.312 | −16.317 | 59.772 | 1.00 | 18.47 |
| ATOM | 14192 | CD2 | PHE | 2129 | 7.101 | −17.186 | 61.862 | 1.00 | 19.74 |
| ATOM | 14193 | CE1 | PHE | 2129 | 5.238 | −17.198 | 59.811 | 1.00 | 19.85 |
| ATOM | 14194 | CE2 | PHE | 2129 | 6.030 | −18.067 | 61.907 | 1.00 | 20.86 |
| ATOM | 14195 | CZ | PHE | 2129 | 5.094 | −18.076 | 60.879 | 1.00 | 20.11 |
| ATOM | 14196 | C | PHE | 2129 | 9.527 | −13.489 | 62.039 | 1.00 | 17.47 |
| ATOM | 14197 | O | PHE | 2129 | 10.064 | −13.799 | 63.097 | 1.00 | 19.26 |
| ATOM | 14198 | N | ALA | 2130 | 10.055 | −12.599 | 61.200 | 1.00 | 18.95 |
| ATOM | 14199 | CA | ALA | 2130 | 11.333 | −11.958 | 61.507 | 1.00 | 17.77 |
| ATOM | 14200 | CB | ALA | 2130 | 11.724 | −11.007 | 60.396 | 1.00 | 17.99 |
| ATOM | 14201 | C | ALA | 2130 | 11.247 | −11.203 | 62.830 | 1.00 | 20.34 |
| ATOM | 14202 | O | ALA | 2130 | 12.143 | −11.284 | 63.673 | 1.00 | 17.97 |
| ATOM | 14203 | N | LYS | 2131 | 10.156 | −10.465 | 63.001 | 1.00 | 19.46 |
| ATOM | 14204 | CA | LYS | 2131 | 9.932 | −9.692 | 64.215 | 1.00 | 22.35 |
| ATOM | 14205 | CB | LYS | 2131 | 8.625 | −8.900 | 64.090 | 1.00 | 23.83 |
| ATOM | 14206 | CG | LYS | 2131 | 8.301 | −7.979 | 65.265 | 1.00 | 26.46 |
| ATOM | 14207 | CD | LYS | 2131 | 9.254 | −6.793 | 65.315 | 1.00 | 29.95 |
| ATOM | 14208 | CE | LYS | 2131 | 8.767 | −5.717 | 66.277 | 1.00 | 30.64 |
| ATOM | 14209 | NZ | LYS | 2131 | 8.650 | −6.214 | 67.675 | 1.00 | 32.08 |
| ATOM | 14210 | C | LYS | 2131 | 9.861 | −10.627 | 65.420 | 1.00 | 21.60 |
| ATOM | 14211 | O | LYS | 2131 | 10.447 | −10.352 | 66.469 | 1.00 | 24.21 |
| ATOM | 14212 | N | LEU | 2132 | 9.149 | −11.737 | 65.259 | 1.00 | 20.48 |
| ATOM | 14213 | CA | LEU | 2132 | 8.981 | −12.712 | 66.331 | 1.00 | 21.79 |
| ATOM | 14214 | CB | LEU | 2132 | 8.000 | −13.805 | 65.892 | 1.00 | 20.32 |
| ATOM | 14215 | CG | LEU | 2132 | 7.523 | −14.833 | 66.930 | 1.00 | 21.91 |
| ATOM | 14216 | CD1 | LEU | 2132 | 6.279 | −15.525 | 66.407 | 1.00 | 21.23 |
| ATOM | 14217 | CD2 | LEU | 2132 | 8.608 | −15.864 | 67.222 | 1.00 | 19.41 |
| ATOM | 14218 | C | LEU | 2132 | 10.303 | −13.335 | 66.764 | 1.00 | 21.65 |
| ATOM | 14219 | O | LEU | 2132 | 10.524 | −13.577 | 67.954 | 1.00 | 20.18 |
| ATOM | 14220 | N | PHE | 2133 | 11.177 | −13.615 | 65.803 | 1.00 | 20.32 |
| ATOM | 14221 | CA | PHE | 2133 | 12.474 | −14.204 | 66.127 | 1.00 | 20.05 |
| ATOM | 14222 | CB | PHE | 2133 | 13.196 | −14.655 | 64.851 | 1.00 | 18.15 |
| ATOM | 14223 | CG | PHE | 2133 | 12.452 | −15.698 | 64.066 | 1.00 | 15.76 |
| ATOM | 14224 | CD1 | PHE | 2133 | 11.618 | −16.610 | 64.707 | 1.00 | 15.98 |
| ATOM | 14225 | CD2 | PHE | 2133 | 12.598 | −15.775 | 62.680 | 1.00 | 16.44 |
| ATOM | 14226 | CE1 | PHE | 2133 | 10.934 | −17.585 | 63.982 | 1.00 | 17.09 |
| ATOM | 14227 | CE2 | PHE | 2133 | 11.922 | −16.746 | 61.947 | 1.00 | 13.66 |
| ATOM | 14228 | CZ | PHE | 2133 | 11.087 | −17.653 | 62.595 | 1.00 | 15.97 |
| ATOM | 14229 | C | PHE | 2133 | 13.344 | −13.213 | 66.875 | 1.00 | 20.41 |
| ATOM | 14230 | O | PHE | 2133 | 13.940 | −13.549 | 67.899 | 1.00 | 22.66 |
| ATOM | 14231 | N | ALA | 2134 | 13.420 | −11.991 | 66.356 | 1.00 | 20.90 |
| ATOM | 14232 | CA | ALA | 2134 | 14.230 | −10.952 | 66.977 | 1.00 | 22.59 |
| ATOM | 14233 | CB | ALA | 2134 | 14.139 | −9.675 | 66.161 | 1.00 | 22.94 |
| ATOM | 14234 | C | ALA | 2134 | 13.799 | −10.690 | 68.417 | 1.00 | 25.23 |
| ATOM | 14235 | O | ALA | 2134 | 14.636 | −10.474 | 69.294 | 1.00 | 25.72 |
| ATOM | 14236 | N | ASP | 2135 | 12.494 | −10.719 | 68.666 | 1.00 | 24.88 |
| ATOM | 14237 | CA | ASP | 2135 | 12.001 | −10.471 | 70.017 | 1.00 | 27.30 |
| ATOM | 14238 | CB | ASP | 2135 | 10.489 | −10.246 | 70.009 | 1.00 | 28.36 |
| ATOM | 14239 | CG | ASP | 2135 | 10.086 | −8.973 | 69.291 | 1.00 | 28.77 |
| ATOM | 14240 | OD1 | ASP | 2135 | 10.963 | −8.137 | 68.993 | 1.00 | 29.20 |
| ATOM | 14241 | OD2 | ASP | 2135 | 8.876 | −8.808 | 69.034 | 1.00 | 28.65 |
| ATOM | 14242 | C | ASP | 2135 | 12.334 | −11.593 | 71.000 | 1.00 | 27.17 |
| ATOM | 14243 | O | ASP | 2135 | 12.518 | −11.342 | 72.189 | 1.00 | 26.00 |
| ATOM | 14244 | N | GLU | 2136 | 12.406 | −12.828 | 70.510 | 1.00 | 27.96 |
| ATOM | 14245 | CA | GLU | 2136 | 12.708 | −13.964 | 71.377 | 1.00 | 28.87 |
| ATOM | 14246 | CB | GLU | 2136 | 12.111 | −15.247 | 70.809 | 1.00 | 27.56 |
| ATOM | 14247 | CG | GLU | 2136 | 10.611 | −15.241 | 70.812 | 1.00 | 30.49 |
| ATOM | 14248 | CD | GLU | 2136 | 10.063 | −14.819 | 72.157 | 1.00 | 31.36 |
| ATOM | 14249 | OE1 | GLU | 2136 | 10.393 | −15.491 | 73.158 | 1.00 | 32.34 |
| ATOM | 14250 | OE2 | GLU | 2136 | 9.319 | −13.816 | 72.207 | 1.00 | 28.17 |
| ATOM | 14251 | C | GLU | 2136 | 14.187 | −14.178 | 71.629 | 1.00 | 29.36 |
| ATOM | 14252 | O | GLU | 2136 | 14.567 | −14.891 | 72.559 | 1.00 | 30.73 |
| ATOM | 14253 | N | GLY | 2137 | 15.029 | −13.581 | 70.794 | 1.00 | 30.45 |
| ATOM | 14254 | CA | GLY | 2137 | 16.461 | −13.738 | 70.988 | 1.00 | 30.66 |
| ATOM | 14255 | C | GLY | 2137 | 17.255 | −14.119 | 69.754 | 1.00 | 29.34 |
| ATOM | 14256 | O | GLY | 2137 | 18.475 | −13.979 | 69.749 | 1.00 | 28.38 |
| ATOM | 14257 | N | LEU | 2138 | 16.572 | −14.607 | 68.720 | 1.00 | 28.35 |
| ATOM | 14258 | CA | LEU | 2138 | 17.229 | −15.003 | 67.471 | 1.00 | 26.93 |
| ATOM | 14259 | CB | LEU | 2138 | 16.304 | −15.885 | 66.634 | 1.00 | 28.35 |
| ATOM | 14260 | CG | LEU | 2138 | 16.435 | −17.385 | 66.848 | 1.00 | 27.76 |
| ATOM | 14261 | CD1 | LEU | 2138 | 15.372 | −18.123 | 66.059 | 1.00 | 24.29 |
| ATOM | 14262 | CD2 | LEU | 2138 | 17.826 | −17.827 | 66.426 | 1.00 | 29.18 |
| ATOM | 14263 | C | LEU | 2138 | 17.590 | −13.765 | 66.674 | 1.00 | 27.25 |
| ATOM | 14264 | O | LEU | 2138 | 16.708 | −13.065 | 66.183 | 1.00 | 27.95 |
| ATOM | 14265 | N | ASN | 2139 | 18.886 | −13.508 | 66.536 | 1.00 | 24.39 |
| ATOM | 14266 | CA | ASN | 2139 | 19.344 | −12.333 | 65.818 | 1.00 | 23.70 |
| ATOM | 14267 | CB | ASN | 2139 | 20.224 | −11.464 | 66.722 | 1.00 | 27.76 |
| ATOM | 14268 | CG | ASN | 2139 | 19.509 | −11.026 | 67.982 | 1.00 | 31.16 |
| ATOM | 14269 | OD1 | ASN | 2139 | 18.415 | −10.458 | 67.924 | 1.00 | 34.02 |
| ATOM | 14270 | ND2 | ASN | 2139 | 20.125 | −11.279 | 69.130 | 1.00 | 33.28 |
| ATOM | 14271 | C | ASN | 2139 | 20.112 | −12.664 | 64.552 | 1.00 | 20.12 |
| ATOM | 14272 | O | ASN | 2139 | 20.800 | −11.804 | 64.016 | 1.00 | 21.01 |
| ATOM | 14273 | N | VAL | 2140 | 20.020 | −13.905 | 64.089 | 1.00 | 17.90 |
| ATOM | 14274 | CA | VAL | 2140 | 20.703 | −14.304 | 62.860 | 1.00 | 17.14 |
| ATOM | 14275 | CB | VAL | 2140 | 21.860 | −15.287 | 63.124 | 1.00 | 19.66 |
| ATOM | 14276 | CG1 | VAL | 2140 | 22.638 | −15.512 | 61.834 | 1.00 | 17.34 |
| ATOM | 14277 | CG2 | VAL | 2140 | 22.770 | −14.735 | 64.215 | 1.00 | 21.32 |
| ATOM | 14278 | C | VAL | 2140 | 19.682 | −14.991 | 61.969 | 1.00 | 14.62 |
| ATOM | 14279 | O | VAL | 2140 | 19.224 | −16.085 | 62.273 | 1.00 | 14.94 |
| ATOM | 14280 | N | MET | 2141 | 19.324 | −14.338 | 60.872 | 1.00 | 15.56 |
| ATOM | 14281 | CA | MET | 2141 | 18.332 | −14.888 | 59.971 | 1.00 | 15.34 |
| ATOM | 14282 | CB | MET | 2141 | 17.066 | −14.034 | 60.024 | 1.00 | 17.74 |
| ATOM | 14283 | CG | MET | 2141 | 16.383 | −14.068 | 61.383 | 1.00 | 20.01 |
| ATOM | 14284 | SD | MET | 2141 | 15.060 | −12.883 | 61.483 | 1.00 | 22.65 |
| ATOM | 14285 | CE | MET | 2141 | 15.531 | −11.942 | 62.941 | 1.00 | 20.39 |
| ATOM | 14286 | C | MET | 2141 | 18.827 | −14.989 | 58.548 | 1.00 | 14.81 |
| ATOM | 14287 | O | MET | 2141 | 19.577 | −14.136 | 58.069 | 1.00 | 14.45 |
| ATOM | 14288 | N | LEU | 2142 | 18.393 | −16.040 | 57.869 | 1.00 | 15.04 |
| ATOM | 14289 | CA | LEU | 2142 | 18.781 | −16.250 | 56.483 | 1.00 | 15.97 |
| ATOM | 14290 | CB | LEU | 2142 | 19.477 | −17.609 | 56.352 | 1.00 | 17.78 |
| ATOM | 14291 | CG | LEU | 2142 | 19.965 | −18.146 | 54.997 | 1.00 | 23.22 |
| ATOM | 14292 | CD1 | LEU | 2142 | 18.838 | −18.861 | 54.306 | 1.00 | 25.80 |
| ATOM | 14293 | CD2 | LEU | 2142 | 20.536 | −17.032 | 54.136 | 1.00 | 22.00 |
| ATOM | 14294 | C | LEU | 2142 | 17.555 | −16.182 | 55.572 | 1.00 | 14.69 |
| ATOM | 14295 | O | LEU | 2142 | 16.570 | −16.886 | 55.781 | 1.00 | 14.54 |
| ATOM | 14296 | N | VAL | 2143 | 17.626 | −15.299 | 54.582 | 1.00 | 13.79 |
| ATOM | 14297 | CA | VAL | 2143 | 16.567 | −15.156 | 53.596 | 1.00 | 14.55 |
| ATOM | 14298 | CB | VAL | 2143 | 16.390 | −13.691 | 53.162 | 1.00 | 15.17 |
| ATOM | 14299 | CG1 | VAL | 2143 | 15.300 | −13.584 | 52.093 | 1.00 | 17.16 |
| ATOM | 14300 | CG2 | VAL | 2143 | 16.006 | −12.837 | 54.379 | 1.00 | 16.68 |
| ATOM | 14301 | C | VAL | 2143 | 17.126 | −15.985 | 52.450 | 1.00 | 14.62 |
| ATOM | 14302 | O | VAL | 2143 | 17.824 | −15.466 | 51.583 | 1.00 | 15.21 |
| ATOM | 14303 | N | GLY | 2144 | 16.843 | −17.283 | 52.474 | 1.00 | 16.51 |
| ATOM | 14304 | CA | GLY | 2144 | 17.379 | −18.166 | 51.454 | 1.00 | 16.80 |
| ATOM | 14305 | C | GLY | 2144 | 16.441 | −18.554 | 50.339 | 1.00 | 16.58 |
| ATOM | 14306 | O | GLY | 2144 | 15.221 | −18.396 | 50.455 | 1.00 | 15.97 |
| ATOM | 14307 | N | ASP | 2145 | 17.009 | −19.077 | 49.254 | 1.00 | 15.26 |
| ATOM | 14308 | CA | ASP | 2145 | 16.193 | −19.474 | 48.107 | 1.00 | 16.47 |
| ATOM | 14309 | CB | ASP | 2145 | 17.066 | −19.746 | 46.875 | 1.00 | 16.11 |
| ATOM | 14310 | CG | ASP | 2145 | 18.084 | −20.849 | 47.095 | 1.00 | 17.05 |
| ATOM | 14311 | OD1 | ASP | 2145 | 18.119 | −21.434 | 48.182 | 1.00 | 15.53 |
| ATOM | 14312 | OD2 | ASP | 2145 | 18.853 | −21.122 | 46.159 | 1.00 | 20.23 |
| ATOM | 14313 | C | ASP | 2145 | 15.289 | −20.667 | 48.406 | 1.00 | 14.31 |
| ATOM | 14314 | O | ASP | 2145 | 14.536 | −21.124 | 47.542 | 1.00 | 13.80 |
| ATOM | 14315 | N | SER | 2146 | 15.350 | −21.158 | 49.642 | 1.00 | 15.43 |
| ATOM | 14316 | CA | SER | 2146 | 14.475 | −22.251 | 50.051 | 1.00 | 13.71 |
| ATOM | 14317 | CB | SER | 2146 | 14.764 | −22.668 | 51.498 | 1.00 | 14.86 |
| ATOM | 14318 | OG | SER | 2146 | 14.708 | −21.562 | 52.384 | 1.00 | 15.94 |
| ATOM | 14319 | C | SER | 2146 | 13.050 | −21.724 | 49.938 | 1.00 | 13.48 |
| ATOM | 14320 | O | SER | 2146 | 12.086 | −22.493 | 49.838 | 1.00 | 13.05 |
| ATOM | 14321 | N | LEU | 2147 | 12.911 | −20.403 | 49.960 | 1.00 | 13.53 |
| ATOM | 14322 | CA | LEU | 2147 | 11.580 | −19.798 | 49.848 | 1.00 | 15.06 |
| ATOM | 14323 | CB | LEU | 2147 | 11.676 | −18.270 | 49.964 | 1.00 | 14.88 |
| ATOM | 14324 | CG | LEU | 2147 | 12.398 | −17.474 | 48.868 | 1.00 | 15.50 |
| ATOM | 14325 | CD1 | LEU | 2147 | 11.425 | −17.211 | 47.718 | 1.00 | 12.97 |
| ATOM | 14326 | CD2 | LEU | 2147 | 12.914 | −16.144 | 49.442 | 1.00 | 14.61 |
| ATOM | 14327 | C | LEU | 2147 | 10.902 | −20.193 | 48.532 | 1.00 | 15.02 |
| ATOM | 14328 | O | LEU | 2147 | 9.674 | −20.120 | 48.406 | 1.00 | 13.70 |
| ATOM | 14329 | N | GLY | 2148 | 11.699 | −20.618 | 47.556 | 1.00 | 13.80 |
| ATOM | 14330 | CA | GLY | 2148 | 11.129 | −21.027 | 46.282 | 1.00 | 14.73 |
| ATOM | 14331 | C | GLY | 2148 | 10.168 | −22.193 | 46.449 | 1.00 | 15.52 |
| ATOM | 14332 | O | GLY | 2148 | 9.240 | −22.374 | 45.664 | 1.00 | 15.06 |
| ATOM | 14333 | N | MET | 2149 | 10.384 | −22.984 | 47.490 | 1.00 | 16.51 |
| ATOM | 14334 | CA | MET | 2149 | 9.543 | −24.142 | 47.739 | 1.00 | 18.26 |
| ATOM | 14335 | CB | MET | 2149 | 10.420 | −25.344 | 48.105 | 1.00 | 19.59 |
| ATOM | 14336 | CG | MET | 2149 | 11.372 | −25.737 | 46.979 | 1.00 | 23.21 |
| ATOM | 14337 | SD | MET | 2149 | 12.577 | −27.009 | 47.403 | 1.00 | 26.89 |
| ATOM | 14338 | CE | MET | 2149 | 11.515 | −28.406 | 47.735 | 1.00 | 27.42 |
| ATOM | 14339 | C | MET | 2149 | 8.525 | −23.867 | 48.836 | 1.00 | 17.57 |
| ATOM | 14340 | O | MET | 2149 | 7.330 | −24.072 | 48.643 | 1.00 | 18.80 |
| ATOM | 14341 | N | THR | 2150 | 9.000 | −23.374 | 49.974 | 1.00 | 17.41 |
| ATOM | 14342 | CA | THR | 2150 | 8.125 | −23.094 | 51.106 | 1.00 | 19.06 |
| ATOM | 14343 | CB | THR | 2150 | 8.947 | −22.896 | 52.395 | 1.00 | 20.48 |
| ATOM | 14344 | OG1 | THR | 2150 | 8.052 | −22.760 | 53.500 | 1.00 | 28.01 |
| ATOM | 14345 | CG2 | THR | 2150 | 9.819 | −21.652 | 52.296 | 1.00 | 16.62 |
| ATOM | 14346 | C | THR | 2150 | 7.189 | −21.893 | 50.948 | 1.00 | 19.09 |
| ATOM | 14347 | O | THR | 2150 | 6.085 | −21.890 | 51.495 | 1.00 | 19.04 |
| ATOM | 14348 | N | VAL | 2151 | 7.631 | −20.878 | 50.212 | 1.00 | 15.32 |
| ATOM | 14349 | CA | VAL | 2151 | 6.816 | −19.688 | 50.000 | 1.00 | 15.78 |
| ATOM | 14350 | CB | VAL | 2151 | 7.660 | −18.400 | 50.167 | 1.00 | 14.07 |
| ATOM | 14351 | CG1 | VAL | 2151 | 6.850 | −17.167 | 49.740 | 1.00 | 14.82 |
| ATOM | 14352 | CG2 | VAL | 2151 | 8.092 | −18.258 | 51.629 | 1.00 | 16.78 |
| ATOM | 14353 | C | VAL | 2151 | 6.122 | −19.683 | 48.634 | 1.00 | 15.00 |
| ATOM | 14354 | O | VAL | 2151 | 4.892 | −19.573 | 48.554 | 1.00 | 12.68 |
| ATOM | 14355 | N | GLN | 2152 | 6.898 | −19.827 | 47.562 | 1.00 | 12.81 |
| ATOM | 14356 | CA | GLN | 2152 | 6.332 | −19.819 | 46.205 | 1.00 | 12.29 |
| ATOM | 14357 | CB | GLN | 2152 | 7.413 | −19.435 | 45.195 | 1.00 | 9.78 |
| ATOM | 14358 | CG | GLN | 2152 | 8.125 | −18.150 | 45.545 | 1.00 | 11.69 |
| ATOM | 14359 | CD | GLN | 2152 | 9.274 | −17.866 | 44.616 | 1.00 | 12.80 |
| ATOM | 14360 | OE1 | GLN | 2152 | 9.733 | −18.750 | 43.901 | 1.00 | 13.02 |
| ATOM | 14361 | NE2 | GLN | 2152 | 9.765 | −16.630 | 44.639 | 1.00 | 10.46 |
| ATOM | 14362 | C | GLN | 2152 | 5.673 | −21.127 | 45.773 | 1.00 | 13.36 |
| ATOM | 14363 | O | GLN | 2152 | 4.812 | −21.127 | 44.897 | 1.00 | 13.82 |
| ATOM | 14364 | N | GLY | 2153 | 6.089 | −22.244 | 46.368 | 1.00 | 14.36 |
| ATOM | 14365 | CA | GLY | 2153 | 5.485 | −23.517 | 46.020 | 1.00 | 13.48 |
| ATOM | 14366 | C | GLY | 2153 | 6.071 | −24.295 | 44.852 | 1.00 | 14.85 |
| ATOM | 14367 | O | GLY | 2153 | 5.409 | −25.187 | 44.308 | 1.00 | 13.64 |
| ATOM | 14368 | N | HIS | 2154 | 7.301 | −23.974 | 44.464 | 1.00 | 14.25 |
| ATOM | 14369 | CA | HIS | 2154 | 7.965 | −24.675 | 43.362 | 1.00 | 16.22 |
| ATOM | 14370 | CB | HIS | 2154 | 9.061 | −23.799 | 42.755 | 1.00 | 14.89 |
| ATOM | 14371 | CG | HIS | 2154 | 8.556 | −22.555 | 42.100 | 1.00 | 16.52 |
| ATOM | 14372 | CD2 | HIS | 2154 | 8.719 | −21.251 | 42.426 | 1.00 | 15.21 |
| ATOM | 14373 | ND1 | HIS | 2154 | 7.799 | −22.572 | 40.949 | 1.00 | 16.03 |
| ATOM | 14374 | CE1 | HIS | 2154 | 7.519 | −21.332 | 40.595 | 1.00 | 18.56 |
| ATOM | 14375 | NE2 | HIS | 2154 | 8.066 | −20.512 | 41.474 | 1.00 | 18.50 |
| ATOM | 14376 | C | HIS | 2154 | 8.586 | −25.991 | 43.836 | 1.00 | 17.17 |
| ATOM | 14377 | O | HIS | 2154 | 8.770 | −26.202 | 45.032 | 1.00 | 17.56 |
| ATOM | 14378 | N | ASP | 2155 | 8.913 | −26.868 | 42.889 | 1.00 | 18.27 |
| ATOM | 14379 | CA | ASP | 2155 | 9.507 | −28.163 | 43.211 | 1.00 | 21.07 |
| ATOM | 14380 | CB | ASP | 2155 | 9.344 | −29.141 | 42.041 | 1.00 | 26.36 |
| ATOM | 14381 | CG | ASP | 2155 | 10.020 | −28.659 | 40.771 | 1.00 | 30.40 |
| ATOM | 14382 | OD1 | ASP | 2155 | 11.238 | −28.378 | 40.795 | 1.00 | 35.46 |
| ATOM | 14383 | OD2 | ASP | 2155 | 9.331 | −28.562 | 39.727 | 1.00 | 38.63 |
| ATOM | 14384 | C | ASP | 2155 | 10.986 | −28.060 | 43.568 | 1.00 | 18.34 |
| ATOM | 14385 | O | ASP | 2155 | 11.600 | −29.037 | 43.990 | 1.00 | 19.68 |
| ATOM | 14386 | N | SER | 2156 | 11.559 | −26.878 | 43.387 | 1.00 | 17.56 |
| ATOM | 14387 | CA | SER | 2156 | 12.968 | −26.667 | 43.692 | 1.00 | 14.88 |
| ATOM | 14388 | CB | SER | 2156 | 13.847 | −27.160 | 42.535 | 1.00 | 15.97 |
| ATOM | 14389 | OG | SER | 2156 | 13.813 | −26.262 | 41.438 | 1.00 | 12.59 |
| ATOM | 14390 | C | SER | 2156 | 13.215 | −25.187 | 43.923 | 1.00 | 15.41 |
| ATOM | 14391 | O | SER | 2156 | 12.300 | −24.372 | 43.791 | 1.00 | 14.28 |
| ATOM | 14392 | N | THR | 2157 | 14.449 | −24.833 | 44.263 | 1.00 | 13.49 |
| ATOM | 14393 | CA | THR | 2157 | 14.776 | −23.433 | 44.514 | 1.00 | 14.04 |
| ATOM | 14394 | CB | THR | 2157 | 15.901 | −23.292 | 45.558 | 1.00 | 14.05 |
| ATOM | 14395 | OG1 | THR | 2157 | 17.117 | −23.825 | 45.018 | 1.00 | 16.03 |
| ATOM | 14396 | CG2 | THR | 2157 | 15.550 | −24.034 | 46.839 | 1.00 | 16.22 |
| ATOM | 14397 | C | THR | 2157 | 15.219 | −22.668 | 43.270 | 1.00 | 13.58 |
| ATOM | 14398 | O | THR | 2157 | 15.329 | −21.440 | 43.313 | 1.00 | 12.41 |
| ATOM | 14399 | N | LEU | 2158 | 15.450 | −23.374 | 42.162 | 1.00 | 13.34 |
| ATOM | 14400 | CA | LEU | 2158 | 15.926 | −22.722 | 40.937 | 1.00 | 12.31 |
| ATOM | 14401 | CB | LEU | 2158 | 16.087 | −23.761 | 39.808 | 1.00 | 14.42 |
| ATOM | 14402 | CG | LEU | 2158 | 17.337 | −24.646 | 39.959 | 1.00 | 16.63 |
| ATOM | 14403 | CD1 | LEU | 2158 | 17.106 | −25.616 | 41.104 | 1.00 | 17.91 |
| ATOM | 14404 | CD2 | LEU | 2158 | 17.631 | −25.409 | 38.669 | 1.00 | 18.40 |
| ATOM | 14405 | C | LEU | 2158 | 15.166 | −21.500 | 40.413 | 1.00 | 12.03 |
| ATOM | 14406 | O | LEU | 2158 | 15.779 | −20.523 | 40.004 | 1.00 | 11.95 |
| ATOM | 14407 | N | PRO | 2159 | 13.825 | −21.526 | 40.437 | 1.00 | 11.73 |
| ATOM | 14408 | CD | PRO | 2159 | 12.922 | −22.627 | 40.802 | 1.00 | 13.77 |
| ATOM | 14409 | CA | PRO | 2159 | 13.060 | −20.378 | 39.941 | 1.00 | 11.00 |
| ATOM | 14410 | CB | PRO | 2159 | 11.629 | −20.913 | 39.914 | 1.00 | 13.29 |
| ATOM | 14411 | CG | PRO | 2159 | 11.619 | −21.891 | 41.005 | 1.00 | 17.80 |
| ATOM | 14412 | C | PRO | 2159 | 13.181 | −19.078 | 40.737 | 1.00 | 8.57 |
| ATOM | 14413 | O | PRO | 2159 | 12.796 | −18.016 | 40.253 | 1.00 | 10.80 |
| ATOM | 14414 | N | VAL | 2160 | 13.717 | −19.147 | 41.949 | 1.00 | 9.75 |
| ATOM | 14415 | CA | VAL | 2160 | 13.856 | −17.939 | 42.764 | 1.00 | 10.64 |
| ATOM | 14416 | CB | VAL | 2160 | 14.334 | −18.295 | 44.191 | 1.00 | 7.98 |
| ATOM | 14417 | CG1 | VAL | 2160 | 14.487 | −17.023 | 45.046 | 1.00 | 8.99 |
| ATOM | 14418 | CG2 | VAL | 2160 | 13.339 | −19.249 | 44.833 | 1.00 | 9.25 |
| ATOM | 14419 | C | VAL | 2160 | 14.827 | −16.956 | 42.120 | 1.00 | 10.30 |
| ATOM | 14420 | O | VAL | 2160 | 15.966 | −17.302 | 41.812 | 1.00 | 12.24 |
| ATOM | 14421 | N | THR | 2161 | 14.373 | −15.718 | 41.940 | 1.00 | 10.56 |
| ATOM | 14422 | CA | THR | 2161 | 15.193 | −14.686 | 41.310 | 1.00 | 13.51 |
| ATOM | 14423 | CB | THR | 2161 | 14.344 | −13.853 | 40.324 | 1.00 | 17.26 |
| ATOM | 14424 | OG1 | THR | 2161 | 13.616 | −14.733 | 39.449 | 1.00 | 18.29 |
| ATOM | 14425 | CG2 | THR | 2161 | 15.234 | −12.954 | 39.478 | 1.00 | 19.77 |
| ATOM | 14426 | C | THR | 2161 | 15.811 | −13.747 | 42.335 | 1.00 | 10.55 |
| ATOM | 14427 | O | THR | 2161 | 15.417 | −13.755 | 43.496 | 1.00 | 11.77 |
| ATOM | 14428 | N | VAL | 2162 | 16.777 | −12.940 | 41.897 | 1.00 | 12.13 |
| ATOM | 14429 | CA | VAL | 2162 | 17.435 | −11.979 | 42.778 | 1.00 | 13.71 |
| ATOM | 14430 | CB | VAL | 2162 | 18.538 | −11.216 | 42.019 | 1.00 | 15.08 |
| ATOM | 14431 | CG1 | VAL | 2162 | 19.051 | −10.057 | 42.858 | 1.00 | 16.93 |
| ATOM | 14432 | CG2 | VAL | 2162 | 19.674 | −12.172 | 41.676 | 1.00 | 15.68 |
| ATOM | 14433 | C | VAL | 2162 | 16.387 | −11.000 | 43.310 | 1.00 | 13.50 |
| ATOM | 14434 | O | VAL | 2162 | 16.436 | −10.571 | 44.469 | 1.00 | 14.68 |
| ATOM | 14435 | N | ALA | 2163 | 15.429 | −10.662 | 42.455 | 1.00 | 13.50 |
| ATOM | 14436 | CA | ALA | 2163 | 14.352 | −9.751 | 42.827 | 1.00 | 12.58 |
| ATOM | 14437 | CB | ALA | 2163 | 13.454 | −9.473 | 41.606 | 1.00 | 13.55 |
| ATOM | 14438 | C | ALA | 2163 | 13.527 | −10.352 | 43.956 | 1.00 | 12.11 |
| ATOM | 14439 | O | ALA | 2163 | 13.094 | −9.637 | 44.872 | 1.00 | 10.50 |
| ATOM | 14440 | N | ASP | 2164 | 13.287 | −11.664 | 43.886 | 1.00 | 10.69 |
| ATOM | 14441 | CA | ASP | 2164 | 12.515 | −12.340 | 44.938 | 1.00 | 12.32 |
| ATOM | 14442 | CB | ASP | 2164 | 12.282 | −13.822 | 44.607 | 1.00 | 10.37 |
| ATOM | 14443 | CG | ASP | 2164 | 11.413 | −14.029 | 43.373 | 1.00 | 14.18 |
| ATOM | 14444 | OD1 | ASP | 2164 | 10.593 | −13.141 | 43.044 | 1.00 | 14.70 |
| ATOM | 14445 | OD2 | ASP | 2164 | 11.546 | −15.107 | 42.745 | 1.00 | 14.94 |
| ATOM | 14446 | C | ASP | 2164 | 13.272 | −12.250 | 46.260 | 1.00 | 12.25 |
| ATOM | 14447 | O | ASP | 2164 | 12.696 | −11.971 | 47.313 | 1.00 | 11.28 |
| ATOM | 14448 | N | ILE | 2165 | 14.574 | −12.500 | 46.212 | 1.00 | 13.30 |
| ATOM | 14449 | CA | ILE | 2165 | 15.363 | −12.429 | 47.433 | 1.00 | 11.13 |
| ATOM | 14450 | CB | ILE | 2165 | 16.827 | −12.815 | 47.165 | 1.00 | 10.95 |
| ATOM | 14451 | CG2 | ILE | 2165 | 17.668 | −12.571 | 48.418 | 1.00 | 12.50 |
| ATOM | 14452 | CG1 | ILE | 2165 | 16.905 | −14.287 | 46.734 | 1.00 | 9.88 |
| ATOM | 14453 | CD1 | ILE | 2165 | 16.581 | −15.294 | 47.819 | 1.00 | 13.15 |
| ATOM | 14454 | C | ILE | 2165 | 15.317 | −11.024 | 48.041 | 1.00 | 11.12 |
| ATOM | 14455 | O | ILE | 2165 | 15.125 | −10.861 | 49.249 | 1.00 | 12.10 |
| ATOM | 14456 | N | ALA | 2166 | 15.478 | −10.016 | 47.195 | 1.00 | 11.76 |
| ATOM | 14457 | CA | ALA | 2166 | 15.471 | −8.618 | 47.634 | 1.00 | 11.59 |
| ATOM | 14458 | CB | ALA | 2166 | 15.742 | −7.710 | 46.439 | 1.00 | 13.22 |
| ATOM | 14459 | C | ALA | 2166 | 14.154 | −8.214 | 48.303 | 1.00 | 13.49 |
| ATOM | 14460 | O | ALA | 2166 | 14.139 | −7.453 | 49.278 | 1.00 | 12.63 |
| ATOM | 14461 | N | TYR | 2167 | 13.056 | −8.704 | 47.738 | 1.00 | 12.03 |
| ATOM | 14462 | CA | TYR | 2167 | 11.710 | −8.448 | 48.230 | 1.00 | 12.41 |
| ATOM | 14463 | CB | TYR | 2167 | 10.711 | −9.189 | 47.334 | 1.00 | 13.48 |
| ATOM | 14464 | CG | TYR | 2167 | 9.281 | −9.135 | 47.805 | 1.00 | 12.75 |
| ATOM | 14465 | CD1 | TYR | 2167 | 8.555 | −7.946 | 47.742 | 1.00 | 15.28 |
| ATOM | 14466 | CE1 | TYR | 2167 | 7.228 | −7.895 | 48.153 | 1.00 | 14.62 |
| ATOM | 14467 | CD2 | TYR | 2167 | 8.645 | −10.276 | 48.299 | 1.00 | 13.65 |
| ATOM | 14468 | CE2 | TYR | 2167 | 7.316 | −10.233 | 48.715 | 1.00 | 13.81 |
| ATOM | 14469 | CZ | TYR | 2167 | 6.617 | −9.038 | 48.637 | 1.00 | 13.06 |
| ATOM | 14470 | OH | TYR | 2167 | 5.303 | −8.988 | 49.050 | 1.00 | 15.51 |
| ATOM | 14471 | C | TYR | 2167 | 11.578 | −8.966 | 49.660 | 1.00 | 13.47 |
| ATOM | 14472 | O | TYR | 2167 | 11.200 | −8.242 | 50.585 | 1.00 | 14.07 |
| ATOM | 14473 | N | HIS | 2168 | 11.902 | −10.241 | 49.824 | 1.00 | 13.89 |
| ATOM | 14474 | CA | HIS | 2168 | 11.823 | −10.890 | 51.120 | 1.00 | 13.79 |
| ATOM | 14475 | CB | HIS | 2168 | 11.943 | −12.403 | 50.929 | 1.00 | 12.97 |
| ATOM | 14476 | CG | HIS | 2168 | 10.731 | −13.014 | 50.289 | 1.00 | 14.62 |
| ATOM | 14477 | CD2 | HIS | 2168 | 10.491 | −13.400 | 49.012 | 1.00 | 13.57 |
| ATOM | 14478 | ND1 | HIS | 2168 | 9.554 | −13.220 | 50.976 | 1.00 | 13.84 |
| ATOM | 14479 | CE1 | HIS | 2168 | 8.641 | −13.706 | 50.152 | 1.00 | 15.74 |
| ATOM | 14480 | NE2 | HIS | 2168 | 9.185 | −13.825 | 48.954 | 1.00 | 11.68 |
| ATOM | 14481 | C | HIS | 2168 | 12.870 | −10.356 | 52.104 | 1.00 | 14.35 |
| ATOM | 14482 | O | HIS | 2168 | 12.634 | −10.331 | 53.313 | 1.00 | 14.35 |
| ATOM | 14483 | N | THR | 2169 | 14.011 | −9.913 | 51.585 | 1.00 | 14.47 |
| ATOM | 14484 | CA | THR | 2169 | 15.068 | −9.369 | 52.428 | 1.00 | 14.38 |
| ATOM | 14485 | CB | THR | 2169 | 16.367 | −9.150 | 51.611 | 1.00 | 15.28 |
| ATOM | 14486 | OG1 | THR | 2169 | 16.949 | −10.425 | 51.308 | 1.00 | 12.29 |
| ATOM | 14487 | CG2 | THR | 2169 | 17.369 | −8.307 | 52.395 | 1.00 | 14.61 |
| ATOM | 14488 | C | THR | 2169 | 14.617 | −8.050 | 53.079 | 1.00 | 14.47 |
| ATOM | 14489 | O | THR | 2169 | 14.784 | −7.857 | 54.288 | 1.00 | 14.08 |
| ATOM | 14490 | N | ALA | 2170 | 14.025 | −7.155 | 52.294 | 1.00 | 13.56 |
| ATOM | 14491 | CA | ALA | 2170 | 13.567 | −5.886 | 52.840 | 1.00 | 13.22 |
| ATOM | 14492 | CB | ALA | 2170 | 13.008 | −4.999 | 51.723 | 1.00 | 12.46 |
| ATOM | 14493 | C | ALA | 2170 | 12.498 | −6.141 | 53.905 | 1.00 | 15.28 |
| ATOM | 14494 | O | ALA | 2170 | 12.467 | −5.471 | 54.946 | 1.00 | 14.21 |
| ATOM | 14495 | N | ALA | 2171 | 11.635 | −7.123 | 53.657 | 1.00 | 15.33 |
| ATOM | 14496 | CA | ALA | 2171 | 10.580 | −7.447 | 54.613 | 1.00 | 15.79 |
| ATOM | 14497 | CB | ALA | 2171 | 9.646 | −8.500 | 54.033 | 1.00 | 15.59 |
| ATOM | 14498 | C | ALA | 2171 | 11.163 | −7.932 | 55.936 | 1.00 | 16.82 |
| ATOM | 14499 | O | ALA | 2171 | 10.733 | −7.500 | 57.010 | 1.00 | 16.97 |
| ATOM | 14500 | N | VAL | 2172 | 12.146 | −8.824 | 55.868 | 1.00 | 16.63 |
| ATOM | 14501 | CA | VAL | 2172 | 12.771 | −9.341 | 57.085 | 1.00 | 16.23 |
| ATOM | 14502 | CB | VAL | 2172 | 13.769 | −10.494 | 56.762 | 1.00 | 17.51 |
| ATOM | 14503 | CG1 | VAL | 2172 | 14.616 | −10.818 | 57.979 | 1.00 | 14.14 |
| ATOM | 14504 | CG2 | VAL | 2172 | 12.999 | −11.742 | 56.338 | 1.00 | 16.91 |
| ATOM | 14505 | C | VAL | 2172 | 13.484 | −8.213 | 57.831 | 1.00 | 16.90 |
| ATOM | 14506 | O | VAL | 2172 | 13.432 | −8.155 | 59.063 | 1.00 | 18.87 |
| ATOM | 14507 | N | ARG | 2173 | 14.138 | −7.321 | 57.089 | 1.00 | 16.84 |
| ATOM | 14508 | CA | ARG | 2173 | 14.860 | −6.195 | 57.689 | 1.00 | 17.87 |
| ATOM | 14509 | CB | ARG | 2173 | 15.594 | −5.380 | 56.613 | 1.00 | 18.06 |
| ATOM | 14510 | CG | ARG | 2173 | 16.334 | −4.149 | 57.156 | 1.00 | 17.82 |
| ATOM | 14511 | CD | ARG | 2173 | 17.239 | −4.512 | 58.340 | 1.00 | 19.45 |
| ATOM | 14512 | NE | ARG | 2173 | 18.482 | −5.163 | 57.929 | 1.00 | 18.22 |
| ATOM | 14513 | CZ | ARG | 2173 | 19.265 | −5.871 | 58.740 | 1.00 | 18.73 |
| ATOM | 14514 | NH1 | ARG | 2173 | 18.946 | −6.037 | 60.017 | 1.00 | 17.92 |
| ATOM | 14515 | NH2 | ARG | 2173 | 20.375 | −6.418 | 58.274 | 1.00 | 17.07 |
| ATOM | 14516 | C | ARG | 2173 | 13.915 | −5.283 | 58.467 | 1.00 | 19.95 |
| ATOM | 14517 | O | ARG | 2173 | 14.280 | −4.753 | 59.523 | 1.00 | 18.61 |
| ATOM | 14518 | N | ARG | 2174 | 12.707 | −5.089 | 57.945 | 1.00 | 18.95 |
| ATOM | 14519 | CA | ARG | 2174 | 11.722 | −4.260 | 58.638 | 1.00 | 20.06 |
| ATOM | 14520 | CB | ARG | 2174 | 10.477 | −4.041 | 57.774 | 1.00 | 18.51 |
| ATOM | 14521 | CG | ARG | 2174 | 10.726 | −3.306 | 56.483 | 1.00 | 20.20 |
| ATOM | 14522 | CD | ARG | 2174 | 9.422 | −2.876 | 55.813 | 1.00 | 22.84 |
| ATOM | 14523 | NE | ARG | 2174 | 9.707 | −2.195 | 54.558 | 1.00 | 24.12 |
| ATOM | 14524 | CZ | ARG | 2174 | 9.783 | −2.796 | 53.375 | 1.00 | 26.27 |
| ATOM | 14525 | NH1 | ARG | 2174 | 9.576 | −4.103 | 53.268 | 1.00 | 23.46 |
| ATOM | 14526 | NH2 | ARG | 2174 | 10.109 | −2.092 | 52.302 | 1.00 | 28.61 |
| ATOM | 14527 | C | ARG | 2174 | 11.319 | −4.976 | 59.916 | 1.00 | 19.12 |
| ATOM | 14528 | O | ARG | 2174 | 11.045 | −4.344 | 60.932 | 1.00 | 22.06 |
| ATOM | 14529 | N | GLY | 2175 | 11.289 | −6.304 | 59.863 | 1.00 | 18.76 |
| ATOM | 14530 | CA | GLY | 2175 | 10.910 | −7.080 | 61.034 | 1.00 | 19.18 |
| ATOM | 14531 | C | GLY | 2175 | 11.974 | −7.136 | 62.117 | 1.00 | 19.83 |
| ATOM | 14532 | O | GLY | 2175 | 11.662 | −7.173 | 63.311 | 1.00 | 19.15 |
| ATOM | 14533 | N | ALA | 2176 | 13.235 | −7.126 | 61.695 | 1.00 | 17.37 |
| ATOM | 14534 | CA | ALA | 2176 | 14.365 | −7.203 | 62.619 | 1.00 | 19.31 |
| ATOM | 14535 | CB | ALA | 2176 | 14.875 | −8.644 | 62.690 | 1.00 | 20.30 |
| ATOM | 14536 | C | ALA | 2176 | 15.481 | −6.278 | 62.155 | 1.00 | 20.49 |
| ATOM | 14537 | O | ALA | 2176 | 16.462 | −6.728 | 61.560 | 1.00 | 20.07 |
| ATOM | 14538 | N | PRO | 2177 | 15.354 | −4.972 | 62.444 | 1.00 | 21.58 |
| ATOM | 14539 | CD | PRO | 2177 | 14.298 | −4.390 | 63.294 | 1.00 | 22.88 |
| ATOM | 14540 | CA | PRO | 2177 | 16.328 | −3.943 | 62.064 | 1.00 | 22.64 |
| ATOM | 14541 | CB | PRO | 2177 | 15.653 | −2.655 | 62.514 | 1.00 | 22.85 |
| ATOM | 14542 | CG | PRO | 2177 | 14.934 | −3.088 | 63.739 | 1.00 | 25.21 |
| ATOM | 14543 | C | PRO | 2177 | 17.734 | −4.080 | 62.631 | 1.00 | 22.77 |
| ATOM | 14544 | O | PRO | 2177 | 18.663 | −3.437 | 62.139 | 1.00 | 23.87 |
| ATOM | 14545 | N | ASN | 2178 | 17.902 | −4.910 | 63.654 | 1.00 | 22.77 |
| ATOM | 14546 | CA | ASN | 2178 | 19.222 | −5.085 | 64.255 | 1.00 | 24.54 |
| ATOM | 14547 | CB | ASN | 2178 | 19.166 | −4.740 | 65.747 | 1.00 | 28.89 |
| ATOM | 14548 | CG | ASN | 2178 | 18.808 | −3.287 | 65.993 | 1.00 | 31.12 |
| ATOM | 14549 | OD1 | ASN | 2178 | 19.380 | −2.389 | 65.380 | 1.00 | 32.75 |
| ATOM | 14550 | ND2 | ASN | 2178 | 17.861 | −3.049 | 66.897 | 1.00 | 35.74 |
| ATOM | 14551 | C | ASN | 2178 | 19.814 | −6.482 | 64.078 | 1.00 | 22.89 |
| ATOM | 14552 | O | ASN | 2178 | 20.908 | −6.771 | 64.573 | 1.00 | 21.60 |
| ATOM | 14553 | N | CYS | 2179 | 19.105 | −7.343 | 63.359 | 1.00 | 21.20 |
| ATOM | 14554 | CA | CYS | 2179 | 19.578 | −8.708 | 63.156 | 1.00 | 19.54 |
| ATOM | 14555 | CB | CYS | 2179 | 18.419 | −9.616 | 62.719 | 1.00 | 22.19 |
| ATOM | 14556 | SG | CYS | 2179 | 18.150 | −9.714 | 60.895 | 1.00 | 21.61 |
| ATOM | 14557 | C | CYS | 2179 | 20.686 | −8.781 | 62.114 | 1.00 | 18.00 |
| ATOM | 14558 | O | CYS | 2179 | 20.898 | −7.845 | 61.336 | 1.00 | 16.56 |
| ATOM | 14559 | N | LEU | 2180 | 21.427 | −9.882 | 62.144 | 1.00 | 17.26 |
| ATOM | 14560 | CA | LEU | 2180 | 22.466 | −10.121 | 61.147 | 1.00 | 18.03 |
| ATOM | 14561 | CB | LEU | 2180 | 23.559 | −11.046 | 61.684 | 1.00 | 18.50 |
| ATOM | 14562 | CG | LEU | 2180 | 24.622 | −11.437 | 60.655 | 1.00 | 18.21 |
| ATOM | 14563 | CD1 | LEU | 2180 | 25.331 | −10.206 | 60.115 | 1.00 | 19.75 |
| ATOM | 14564 | CD2 | LEU | 2180 | 25.616 | −12.392 | 61.293 | 1.00 | 21.75 |
| ATOM | 14565 | C | LEU | 2180 | 21.647 | −10.853 | 60.092 | 1.00 | 15.89 |
| ATOM | 14566 | O | LEU | 2180 | 21.164 | −11.958 | 60.338 | 1.00 | 15.61 |
| ATOM | 14567 | N | LEU | 2181 | 21.483 | −10.229 | 58.933 | 1.00 | 16.34 |
| ATOM | 14568 | CA | LEU | 2181 | 20.675 | −10.802 | 57.865 | 1.00 | 15.32 |
| ATOM | 14569 | CB | LEU | 2181 | 19.730 | −9.734 | 57.308 | 1.00 | 14.91 |
| ATOM | 14570 | CG | LEU | 2181 | 18.435 | −10.175 | 56.612 | 1.00 | 15.55 |
| ATOM | 14571 | CD1 | LEU | 2181 | 17.575 | −8.936 | 56.395 | 1.00 | 16.38 |
| ATOM | 14572 | CD2 | LEU | 2181 | 18.736 | −10.863 | 55.293 | 1.00 | 20.67 |
| ATOM | 14573 | C | LEU | 2181 | 21.498 | −11.382 | 56.727 | 1.00 | 13.75 |
| ATOM | 14574 | O | LEU | 2181 | 22.225 | −10.657 | 56.053 | 1.00 | 14.71 |
| ATOM | 14575 | N | LEU | 2182 | 21.400 | −12.690 | 56.525 | 1.00 | 13.90 |
| ATOM | 14576 | CA | LEU | 2182 | 22.123 | −13.329 | 55.435 | 1.00 | 15.95 |
| ATOM | 14577 | CB | LEU | 2182 | 22.726 | −14.660 | 55.875 | 1.00 | 15.67 |
| ATOM | 14578 | CG | LEU | 2182 | 24.149 | −14.629 | 56.437 | 1.00 | 20.08 |
| ATOM | 14579 | CD1 | LEU | 2182 | 24.196 | −13.819 | 57.715 | 1.00 | 19.21 |
| ATOM | 14580 | CD2 | LEU | 2182 | 24.609 | −16.065 | 56.686 | 1.00 | 19.28 |
| ATOM | 14581 | C | LEU | 2182 | 21.136 | −13.585 | 54.308 | 1.00 | 15.71 |
| ATOM | 14582 | O | LEU | 2182 | 20.040 | −14.075 | 54.552 | 1.00 | 19.38 |
| ATOM | 14583 | N | ALA | 2183 | 21.508 | −13.246 | 53.081 | 1.00 | 13.91 |
| ATOM | 14584 | CA | ALA | 2183 | 20.614 | −13.486 | 51.962 | 1.00 | 14.60 |
| ATOM | 14585 | CB | ALA | 2183 | 20.126 | −12.169 | 51.386 | 1.00 | 13.61 |
| ATOM | 14586 | C | ALA | 2183 | 21.314 | −14.305 | 50.893 | 1.00 | 14.00 |
| ATOM | 14587 | O | ALA | 2183 | 22.471 | −14.049 | 50.538 | 1.00 | 11.35 |
| ATOM | 14588 | N | ASP | 2184 | 20.609 | −15.309 | 50.388 | 1.00 | 14.08 |
| ATOM | 14589 | CA | ASP | 2184 | 21.169 | −16.162 | 49.346 | 1.00 | 16.54 |
| ATOM | 14590 | CB | ASP | 2184 | 20.341 | −17.439 | 49.180 | 1.00 | 16.86 |
| ATOM | 14591 | CG | ASP | 2184 | 20.733 | −18.535 | 50.138 | 1.00 | 20.98 |
| ATOM | 14592 | OD1 | ASP | 2184 | 21.818 | −18.459 | 50.747 | 1.00 | 22.21 |
| ATOM | 14593 | OD2 | ASP | 2184 | 19.945 | −19.496 | 50.255 | 1.00 | 20.02 |
| ATOM | 14594 | C | ASP | 2184 | 21.177 | −15.479 | 47.997 | 1.00 | 14.79 |
| ATOM | 14595 | O | ASP | 2184 | 20.295 | −14.678 | 47.694 | 1.00 | 15.78 |
| ATOM | 14596 | N | LEU | 2185 | 22.200 | −15.764 | 47.200 | 1.00 | 14.30 |
| ATOM | 14597 | CA | LEU | 2185 | 22.205 | −15.275 | 45.831 | 1.00 | 13.25 |
| ATOM | 14598 | CB | LEU | 2185 | 23.614 | −15.010 | 45.302 | 1.00 | 15.09 |
| ATOM | 14599 | CG | LEU | 2185 | 24.210 | −13.667 | 45.767 | 1.00 | 16.23 |
| ATOM | 14600 | CD1 | LEU | 2185 | 25.463 | −13.358 | 44.938 | 1.00 | 17.48 |
| ATOM | 14601 | CD2 | LEU | 2185 | 23.181 | −12.546 | 45.571 | 1.00 | 14.68 |
| ATOM | 14602 | C | LEU | 2185 | 21.618 | −16.538 | 45.211 | 1.00 | 13.97 |
| ATOM | 14603 | O | LEU | 2185 | 22.118 | −17.652 | 45.436 | 1.00 | 13.10 |
| ATOM | 14604 | N | PRO | 2186 | 20.525 | −16.391 | 44.456 | 1.00 | 14.11 |
| ATOM | 14605 | CD | PRO | 2186 | 19.831 | −15.118 | 44.188 | 1.00 | 15.09 |
| ATOM | 14606 | CA | PRO | 2186 | 19.847 | −17.516 | 43.817 | 1.00 | 14.43 |
| ATOM | 14607 | CB | PRO | 2186 | 18.510 | −16.910 | 43.396 | 1.00 | 15.40 |
| ATOM | 14608 | CG | PRO | 2186 | 18.872 | −15.483 | 43.085 | 1.00 | 15.67 |
| ATOM | 14609 | C | PRO | 2186 | 20.584 | −18.177 | 42.663 | 1.00 | 14.33 |
| ATOM | 14610 | O | PRO | 2186 | 21.688 | −17.782 | 42.302 | 1.00 | 15.03 |
| ATOM | 14611 | N | PHE | 2187 | 19.945 | −19.197 | 42.100 | 1.00 | 13.69 |
| ATOM | 14612 | CA | PHE | 2187 | 20.481 | −19.951 | 40.977 | 1.00 | 12.48 |
| ATOM | 14613 | CB | PHE | 2187 | 19.404 | −20.933 | 40.493 | 1.00 | 10.11 |
| ATOM | 14614 | CG | PHE | 2187 | 19.660 | −21.528 | 39.131 | 1.00 | 13.16 |
| ATOM | 14615 | CD1 | PHE | 2187 | 20.749 | −22.365 | 38.904 | 1.00 | 11.33 |
| ATOM | 14616 | CD2 | PHE | 2187 | 18.751 | −21.300 | 38.088 | 1.00 | 12.80 |
| ATOM | 14617 | CE1 | PHE | 2187 | 20.934 | −22.979 | 37.655 | 1.00 | 14.13 |
| ATOM | 14618 | CE2 | PHE | 2187 | 18.914 | −21.899 | 36.837 | 1.00 | 12.25 |
| ATOM | 14619 | CZ | PHE | 2187 | 20.005 | −22.739 | 36.619 | 1.00 | 13.80 |
| ATOM | 14620 | C | PHE | 2187 | 20.938 | −19.024 | 39.849 | 1.00 | 11.25 |
| ATOM | 14621 | O | PHE | 2187 | 20.224 | −18.098 | 39.452 | 1.00 | 11.41 |
| ATOM | 14622 | N | MET | 2188 | 22.147 | −19.282 | 39.358 | 1.00 | 12.61 |
| ATOM | 14623 | CA | MET | 2188 | 22.762 | −18.531 | 38.275 | 1.00 | 13.77 |
| ATOM | 14624 | CB | MET | 2188 | 22.055 | −18.862 | 36.943 | 1.00 | 13.96 |
| ATOM | 14625 | CG | MET | 2188 | 22.956 | −18.723 | 35.707 | 1.00 | 16.90 |
| ATOM | 14626 | SD | MET | 2188 | 24.400 | −19.853 | 35.851 | 1.00 | 12.86 |
| ATOM | 14627 | CE | MET | 2188 | 23.813 | −21.357 | 35.069 | 1.00 | 19.25 |
| ATOM | 14628 | C | MET | 2188 | 22.778 | −17.015 | 38.519 | 1.00 | 14.59 |
| ATOM | 14629 | O | MET | 2188 | 22.648 | −16.226 | 37.584 | 1.00 | 16.18 |
| ATOM | 14630 | N | ALA | 2189 | 22.933 | −16.599 | 39.775 | 1.00 | 13.29 |
| ATOM | 14631 | CA | ALA | 2189 | 22.986 | −15.167 | 40.099 | 1.00 | 13.72 |
| ATOM | 14632 | CB | ALA | 2189 | 22.112 | −14.864 | 41.333 | 1.00 | 12.70 |
| ATOM | 14633 | C | ALA | 2189 | 24.437 | −14.747 | 40.359 | 1.00 | 14.21 |
| ATOM | 14634 | O | ALA | 2189 | 24.719 | −13.580 | 40.633 | 1.00 | 15.89 |
| ATOM | 14635 | N | TYR | 2190 | 25.357 | −15.708 | 40.278 | 1.00 | 12.28 |
| ATOM | 14636 | CA | TYR | 2190 | 26.789 | −15.433 | 40.483 | 1.00 | 13.07 |
| ATOM | 14637 | CB | TYR | 2190 | 27.175 | −15.714 | 41.946 | 1.00 | 12.91 |
| ATOM | 14638 | CG | TYR | 2190 | 26.689 | −17.035 | 42.515 | 1.00 | 11.62 |
| ATOM | 14639 | CD1 | TYR | 2190 | 27.524 | −18.156 | 42.554 | 1.00 | 11.92 |
| ATOM | 14640 | CE1 | TYR | 2190 | 27.087 | −19.366 | 43.090 | 1.00 | 12.64 |
| ATOM | 14641 | CD2 | TYR | 2190 | 25.396 | −17.158 | 43.027 | 1.00 | 14.06 |
| ATOM | 14642 | CE2 | TYR | 2190 | 24.944 | −18.360 | 43.563 | 1.00 | 13.68 |
| ATOM | 14643 | CZ | TYR | 2190 | 25.793 | −19.461 | 43.593 | 1.00 | 14.72 |
| ATOM | 14644 | OH | TYR | 2190 | 25.349 | −20.652 | 44.128 | 1.00 | 15.24 |
| ATOM | 14645 | C | TYR | 2190 | 27.650 | −16.262 | 39.515 | 1.00 | 14.64 |
| ATOM | 14646 | O | TYR | 2190 | 28.681 | −16.822 | 39.887 | 1.00 | 14.18 |
| ATOM | 14647 | N | ALA | 2191 | 27.194 | −16.316 | 38.267 | 1.00 | 13.79 |
| ATOM | 14648 | CA | ALA | 2191 | 27.843 | −17.062 | 37.194 | 1.00 | 14.38 |
| ATOM | 14649 | CB | ALA | 2191 | 26.929 | −17.077 | 35.976 | 1.00 | 13.74 |
| ATOM | 14650 | C | ALA | 2191 | 29.211 | −16.503 | 36.808 | 1.00 | 13.17 |
| ATOM | 14651 | O | ALA | 2191 | 30.072 | −17.240 | 36.331 | 1.00 | 12.13 |
| ATOM | 14652 | N | THR | 2192 | 29.382 | −15.198 | 36.998 | 1.00 | 12.89 |
| ATOM | 14653 | CA | THR | 2192 | 30.629 | −14.500 | 36.705 | 1.00 | 14.63 |
| ATOM | 14654 | CB | THR | 2192 | 30.560 | −13.704 | 35.386 | 1.00 | 15.14 |
| ATOM | 14655 | OG1 | THR | 2192 | 29.676 | −12.583 | 35.546 | 1.00 | 13.87 |
| ATOM | 14656 | CG2 | THR | 2192 | 30.036 | −14.589 | 34.254 | 1.00 | 15.22 |
| ATOM | 14657 | C | THR | 2192 | 30.828 | −13.495 | 37.835 | 1.00 | 16.27 |
| ATOM | 14658 | O | THR | 2192 | 29.873 | −13.126 | 38.517 | 1.00 | 15.41 |
| ATOM | 14659 | N | PRO | 2193 | 32.073 | −13.042 | 38.053 | 1.00 | 16.19 |
| ATOM | 14660 | CD | PRO | 2193 | 33.343 | −13.538 | 37.490 | 1.00 | 16.85 |
| ATOM | 14661 | CA | PRO | 2193 | 32.315 | −12.073 | 39.124 | 1.00 | 16.55 |
| ATOM | 14662 | CB | PRO | 2193 | 33.803 | −11.792 | 38.992 | 1.00 | 18.64 |
| ATOM | 14663 | CG | PRO | 2193 | 34.343 | −13.133 | 38.552 | 1.00 | 17.11 |
| ATOM | 14664 | C | PRO | 2193 | 31.465 | −10.824 | 38.937 | 1.00 | 15.31 |
| ATOM | 14665 | O | PRO | 2193 | 30.831 | −10.354 | 39.876 | 1.00 | 15.59 |
| ATOM | 14666 | N | GLU | 2194 | 31.454 | −10.296 | 37.718 | 1.00 | 16.75 |
| ATOM | 14667 | CA | GLU | 2194 | 30.687 | −9.098 | 37.419 | 1.00 | 17.91 |
| ATOM | 14668 | CB | GLU | 2194 | 30.858 | −8.711 | 35.948 | 1.00 | 23.31 |
| ATOM | 14669 | CG | GLU | 2194 | 32.215 | −8.092 | 35.630 | 1.00 | 30.85 |
| ATOM | 14670 | CD | GLU | 2194 | 32.409 | −7.831 | 34.149 | 1.00 | 35.36 |
| ATOM | 14671 | OE1 | GLU | 2194 | 31.524 | −7.192 | 33.537 | 1.00 | 39.04 |
| ATOM | 14672 | OE2 | GLU | 2194 | 33.450 | −8.260 | 33.598 | 1.00 | 39.02 |
| ATOM | 14673 | C | GLU | 2194 | 29.206 | −9.245 | 37.757 | 1.00 | 17.81 |
| ATOM | 14674 | O | GLU | 2194 | 28.602 | −8.322 | 38.304 | 1.00 | 17.37 |
| ATOM | 14675 | N | GLN | 2195 | 28.612 | −10.390 | 37.435 | 1.00 | 15.20 |
| ATOM | 14676 | CA | GLN | 2195 | 27.199 | −10.586 | 37.744 | 1.00 | 16.01 |
| ATOM | 14677 | CB | GLN | 2195 | 26.642 | −11.799 | 36.994 | 1.00 | 17.15 |
| ATOM | 14678 | CG | GLN | 2195 | 26.551 | −11.574 | 35.488 | 1.00 | 21.26 |
| ATOM | 14679 | CD | GLN | 2195 | 26.048 | −12.795 | 34.750 | 1.00 | 25.19 |
| ATOM | 14680 | OE1 | GLN | 2195 | 24.964 | −13.304 | 35.038 | 1.00 | 25.51 |
| ATOM | 14681 | NE2 | GLN | 2195 | 26.835 | −13.273 | 33.787 | 1.00 | 28.35 |
| ATOM | 14682 | C | GLN | 2195 | 27.006 | −10.756 | 39.248 | 1.00 | 14.45 |
| ATOM | 14683 | O | GLN | 2195 | 26.010 | −10.300 | 39.798 | 1.00 | 14.86 |
| ATOM | 14684 | N | ALA | 2196 | 27.966 | −11.407 | 39.899 | 1.00 | 12.21 |
| ATOM | 14685 | CA | ALA | 2196 | 27.908 | −11.620 | 41.329 | 1.00 | 12.40 |
| ATOM | 14686 | CB | ALA | 2196 | 29.105 | −12.455 | 41.795 | 1.00 | 13.23 |
| ATOM | 14687 | C | ALA | 2196 | 27.898 | −10.272 | 42.047 | 1.00 | 11.64 |
| ATOM | 14688 | O | ALA | 2196 | 27.146 | −10.072 | 43.010 | 1.00 | 11.62 |
| ATOM | 14689 | N | PHE | 2197 | 28.727 | −9.346 | 41.571 | 1.00 | 11.56 |
| ATOM | 14690 | CA | PHE | 2197 | 28.809 | −8.007 | 42.164 | 1.00 | 13.89 |
| ATOM | 14691 | CB | PHE | 2197 | 29.858 | −7.146 | 41.442 | 1.00 | 13.21 |
| ATOM | 14692 | CG | PHE | 2197 | 31.236 | −7.743 | 41.414 | 1.00 | 12.74 |
| ATOM | 14693 | CD1 | PHE | 2197 | 31.673 | −8.576 | 42.432 | 1.00 | 13.16 |
| ATOM | 14694 | CD2 | PHE | 2197 | 32.106 | −7.448 | 40.368 | 1.00 | 17.01 |
| ATOM | 14695 | CE1 | PHE | 2197 | 32.962 | −9.119 | 42.413 | 1.00 | 16.01 |
| ATOM | 14696 | CE2 | PHE | 2197 | 33.397 | −7.981 | 40.332 | 1.00 | 15.05 |
| ATOM | 14697 | CZ | PHE | 2197 | 33.819 | −8.815 | 41.355 | 1.00 | 15.53 |
| ATOM | 14698 | C | PHE | 2197 | 27.469 | −7.282 | 42.071 | 1.00 | 15.38 |
| ATOM | 14699 | O | PHE | 2197 | 26.976 | −6.713 | 43.047 | 1.00 | 15.31 |
| ATOM | 14700 | N | GLU | 2198 | 26.900 | −7.296 | 40.874 | 1.00 | 16.38 |
| ATOM | 14701 | CA | GLU | 2198 | 25.636 | −6.629 | 40.619 | 1.00 | 18.38 |
| ATOM | 14702 | CB | GLU | 2198 | 25.286 | −6.745 | 39.134 | 1.00 | 21.89 |
| ATOM | 14703 | CG | GLU | 2198 | 24.046 | −5.985 | 38.698 | 1.00 | 28.47 |
| ATOM | 14704 | CD | GLU | 2198 | 24.133 | −4.486 | 38.975 | 1.00 | 31.20 |
| ATOM | 14705 | OE1 | GLU | 2198 | 25.253 | −3.974 | 39.213 | 1.00 | 31.05 |
| ATOM | 14706 | OE2 | GLU | 2198 | 23.074 | −3.823 | 38.939 | 1.00 | 33.83 |
| ATOM | 14707 | C | GLU | 2198 | 24.502 | −7.198 | 41.463 | 1.00 | 16.31 |
| ATOM | 14708 | O | GLU | 2198 | 23.748 | −6.451 | 42.088 | 1.00 | 15.63 |
| ATOM | 14709 | N | ASN | 2199 | 24.390 | −8.520 | 41.488 | 1.00 | 14.90 |
| ATOM | 14710 | CA | ASN | 2199 | 23.331 | −9.173 | 42.234 | 1.00 | 15.15 |
| ATOM | 14711 | CB | ASN | 2199 | 23.176 | −10.619 | 41.748 | 1.00 | 14.55 |
| ATOM | 14712 | CG | ASN | 2199 | 22.703 | −10.687 | 40.306 | 1.00 | 14.12 |
| ATOM | 14713 | OD1 | ASN | 2199 | 21.887 | −9.869 | 39.880 | 1.00 | 13.06 |
| ATOM | 14714 | ND2 | ASN | 2199 | 23.196 | −11.666 | 39.551 | 1.00 | 16.16 |
| ATOM | 14715 | C | ASN | 2199 | 23.537 | −9.112 | 43.737 | 1.00 | 13.30 |
| ATOM | 14716 | O | ASN | 2199 | 22.576 | −8.971 | 44.494 | 1.00 | 11.68 |
| ATOM | 14717 | N | ALA | 2200 | 24.790 | −9.200 | 44.170 | 1.00 | 13.38 |
| ATOM | 14718 | CA | ALA | 2200 | 25.106 | −9.121 | 45.596 | 1.00 | 14.01 |
| ATOM | 14719 | CB | ALA | 2200 | 26.601 | −9.377 | 45.819 | 1.00 | 14.48 |
| ATOM | 14720 | C | ALA | 2200 | 24.738 | −7.720 | 46.092 | 1.00 | 12.03 |
| ATOM | 14721 | O | ALA | 2200 | 24.204 | −7.558 | 47.189 | 1.00 | 13.32 |
| ATOM | 14722 | N | ALA | 2201 | 25.022 | −6.713 | 45.271 | 1.00 | 13.67 |
| ATOM | 14723 | CA | ALA | 2201 | 24.716 | −5.335 | 45.625 | 1.00 | 13.13 |
| ATOM | 14724 | CB | ALA | 2201 | 25.285 | −4.370 | 44.573 | 1.00 | 13.81 |
| ATOM | 14725 | C | ALA | 2201 | 23.206 | −5.132 | 45.751 | 1.00 | 13.16 |
| ATOM | 14726 | O | ALA | 2201 | 22.736 | −4.435 | 46.645 | 1.00 | 15.03 |
| ATOM | 14727 | N | THR | 2202 | 22.446 | −5.743 | 44.853 | 1.00 | 13.60 |
| ATOM | 14728 | CA | THR | 2202 | 20.999 | −5.613 | 44.897 | 1.00 | 11.59 |
| ATOM | 14729 | CB | THR | 2202 | 20.353 | −6.445 | 43.798 | 1.00 | 10.38 |
| ATOM | 14730 | OG1 | THR | 2202 | 20.822 | −5.973 | 42.537 | 1.00 | 11.16 |
| ATOM | 14731 | CG2 | THR | 2202 | 18.836 | −6.331 | 43.848 | 1.00 | 11.71 |
| ATOM | 14732 | C | THR | 2202 | 20.446 | −6.068 | 46.244 | 1.00 | 11.76 |
| ATOM | 14733 | O | THR | 2202 | 19.665 | −5.361 | 46.896 | 1.00 | 12.13 |
| ATOM | 14734 | N | VAL | 2203 | 20.867 | −7.252 | 46.658 | 1.00 | 11.71 |
| ATOM | 14735 | CA | VAL | 2203 | 20.431 | −7.835 | 47.908 | 1.00 | 14.15 |
| ATOM | 14736 | CB | VAL | 2203 | 20.883 | −9.303 | 47.970 | 1.00 | 17.90 |
| ATOM | 14737 | CG1 | VAL | 2203 | 20.327 | −9.969 | 49.200 | 1.00 | 22.76 |
| ATOM | 14738 | CG2 | VAL | 2203 | 20.393 | −10.031 | 46.713 | 1.00 | 21.66 |
| ATOM | 14739 | C | VAL | 2203 | 20.953 | −7.062 | 49.116 | 1.00 | 13.49 |
| ATOM | 14740 | O | VAL | 2203 | 20.251 | −6.904 | 50.122 | 1.00 | 11.71 |
| ATOM | 14741 | N | MET | 2204 | 22.182 | −6.570 | 49.026 | 1.00 | 12.79 |
| ATOM | 14742 | CA | MET | 2204 | 22.743 | −5.798 | 50.136 | 1.00 | 15.57 |
| ATOM | 14743 | CB | MET | 2204 | 24.224 | −5.501 | 49.875 | 1.00 | 16.63 |
| ATOM | 14744 | CG | MET | 2204 | 25.104 | −6.724 | 49.887 | 1.00 | 18.75 |
| ATOM | 14745 | SD | MET | 2204 | 25.263 | −7.508 | 51.497 | 1.00 | 19.13 |
| ATOM | 14746 | CE | MET | 2204 | 26.337 | −6.307 | 52.339 | 1.00 | 18.60 |
| ATOM | 14747 | C | MET | 2204 | 21.963 | −4.497 | 50.308 | 1.00 | 14.20 |
| ATOM | 14748 | O | MET | 2204 | 21.556 | −4.147 | 51.421 | 1.00 | 13.48 |
| ATOM | 14749 | N | ARG | 2205 | 21.739 | −3.785 | 49.208 | 1.00 | 13.14 |
| ATOM | 14750 | CA | ARG | 2205 | 20.995 | −2.527 | 49.284 | 1.00 | 13.16 |
| ATOM | 14751 | CB | ARG | 2205 | 20.887 | −1.887 | 47.898 | 1.00 | 13.73 |
| ATOM | 14752 | CG | ARG | 2205 | 22.229 | −1.512 | 47.283 | 1.00 | 15.34 |
| ATOM | 14753 | CD | ARG | 2205 | 22.044 | −0.529 | 46.141 | 1.00 | 14.72 |
| ATOM | 14754 | NE | ARG | 2205 | 23.263 | −0.355 | 45.349 | 1.00 | 16.85 |
| ATOM | 14755 | CZ | ARG | 2205 | 23.591 | −1.101 | 44.299 | 1.00 | 15.47 |
| ATOM | 14756 | NH1 | ARG | 2205 | 22.791 | −2.083 | 43.900 | 1.00 | 16.64 |
| ATOM | 14757 | NH2 | ARG | 2205 | 24.717 | −0.857 | 43.642 | 1.00 | 16.81 |
| ATOM | 14758 | C | ARG | 2205 | 19.601 | −2.780 | 49.859 | 1.00 | 15.15 |
| ATOM | 14759 | O | ARG | 2205 | 19.033 | −1.919 | 50.530 | 1.00 | 13.24 |
| ATOM | 14760 | N | ALA | 2206 | 19.071 | −3.975 | 49.602 | 1.00 | 13.63 |
| ATOM | 14761 | CA | ALA | 2206 | 17.750 | −4.366 | 50.090 | 1.00 | 14.83 |
| ATOM | 14762 | CB | ALA | 2206 | 17.292 | −5.649 | 49.393 | 1.00 | 14.81 |
| ATOM | 14763 | C | ALA | 2206 | 17.717 | −4.559 | 51.609 | 1.00 | 15.51 |
| ATOM | 14764 | O | ALA | 2206 | 16.645 | −4.607 | 52.209 | 1.00 | 15.01 |
| ATOM | 14765 | N | GLY | 2207 | 18.888 | −4.689 | 52.226 | 1.00 | 14.53 |
| ATOM | 14766 | CA | GLY | 2207 | 18.933 | −4.854 | 53.666 | 1.00 | 14.24 |
| ATOM | 14767 | C | GLY | 2207 | 19.806 | −5.969 | 54.210 | 1.00 | 14.79 |
| ATOM | 14768 | O | GLY | 2207 | 19.965 | −6.098 | 55.430 | 1.00 | 13.55 |
| ATOM | 14769 | N | ALA | 2208 | 20.384 | −6.782 | 53.330 | 1.00 | 13.05 |
| ATOM | 14770 | CA | ALA | 2208 | 21.235 | −7.878 | 53.790 | 1.00 | 13.56 |
| ATOM | 14771 | CB | ALA | 2208 | 21.478 | −8.860 | 52.650 | 1.00 | 14.49 |
| ATOM | 14772 | C | ALA | 2208 | 22.568 | −7.351 | 54.310 | 1.00 | 13.56 |
| ATOM | 14773 | O | ALA | 2208 | 22.982 | −6.257 | 53.946 | 1.00 | 14.11 |
| ATOM | 14774 | N | ASN | 2209 | 23.216 | −8.112 | 55.187 | 1.00 | 14.56 |
| ATOM | 14775 | CA | ASN | 2209 | 24.526 | −7.715 | 55.720 | 1.00 | 16.30 |
| ATOM | 14776 | CB | ASN | 2209 | 24.596 | −7.881 | 57.236 | 1.00 | 17.22 |
| ATOM | 14777 | CG | ASN | 2209 | 23.534 | −7.107 | 57.964 | 1.00 | 15.22 |
| ATOM | 14778 | OD1 | ASN | 2209 | 23.458 | −5.884 | 57.868 | 1.00 | 20.45 |
| ATOM | 14779 | ND2 | ASN | 2209 | 22.707 | −7.821 | 58.711 | 1.00 | 13.67 |
| ATOM | 14780 | C | ASN | 2209 | 25.579 | −8.641 | 55.139 | 1.00 | 16.35 |
| ATOM | 14781 | O | ASN | 2209 | 26.776 | −8.360 | 55.194 | 1.00 | 16.50 |
| ATOM | 14782 | N | MET | 2210 | 25.113 | −9.755 | 54.594 | 1.00 | 16.82 |
| ATOM | 14783 | CA | MET | 2210 | 25.996 | −10.772 | 54.051 | 1.00 | 16.51 |
| ATOM | 14784 | CB | MET | 2210 | 26.474 | −11.661 | 55.197 | 1.00 | 18.41 |
| ATOM | 14785 | CG | MET | 2210 | 27.305 | −12.865 | 54.798 | 1.00 | 19.82 |
| ATOM | 14786 | SD | MET | 2210 | 27.830 | −13.789 | 56.264 | 1.00 | 21.89 |
| ATOM | 14787 | CE | MET | 2210 | 29.505 | −13.207 | 56.418 | 1.00 | 21.07 |
| ATOM | 14788 | C | MET | 2210 | 25.262 | −11.605 | 53.015 | 1.00 | 16.91 |
| ATOM | 14789 | O | MET | 2210 | 24.049 | −11.784 | 53.100 | 1.00 | 14.74 |
| ATOM | 14790 | N | VAL | 2211 | 26.009 | −12.110 | 52.041 | 1.00 | 15.70 |
| ATOM | 14791 | CA | VAL | 2211 | 25.444 | −12.926 | 50.978 | 1.00 | 16.74 |
| ATOM | 14792 | CB | VAL | 2211 | 25.795 | −12.306 | 49.613 | 1.00 | 19.57 |
| ATOM | 14793 | CG1 | VAL | 2211 | 25.664 | −13.330 | 48.525 | 1.00 | 23.64 |
| ATOM | 14794 | CG2 | VAL | 2211 | 24.890 | −11.110 | 49.342 | 1.00 | 18.37 |
| ATOM | 14795 | C | VAL | 2211 | 25.982 | −14.356 | 51.052 | 1.00 | 17.23 |
| ATOM | 14796 | O | VAL | 2211 | 27.151 | −14.570 | 51.388 | 1.00 | 17.13 |
| ATOM | 14797 | N | LYS | 2212 | 25.130 | −15.334 | 50.757 | 1.00 | 18.13 |
| ATOM | 14798 | CA | LYS | 2212 | 25.558 | −16.726 | 50.767 | 1.00 | 17.96 |
| ATOM | 14799 | CB | LYS | 2212 | 24.650 | −17.582 | 51.669 | 1.00 | 17.25 |
| ATOM | 14800 | CG | LYS | 2212 | 25.056 | −19.051 | 51.721 | 1.00 | 17.85 |
| ATOM | 14801 | CD | LYS | 2212 | 24.563 | −19.773 | 52.981 | 1.00 | 19.98 |
| ATOM | 14802 | CE | LYS | 2212 | 23.056 | −19.981 | 52.972 | 1.00 | 20.44 |
| ATOM | 14803 | NZ | LYS | 2212 | 22.617 | −20.726 | 51.751 | 1.00 | 19.76 |
| ATOM | 14804 | C | LYS | 2212 | 25.527 | −17.254 | 49.339 | 1.00 | 19.04 |
| ATOM | 14805 | O | LYS | 2212 | 24.612 | −16.947 | 48.568 | 1.00 | 18.54 |
| ATOM | 14806 | N | ILE | 2213 | 26.548 | −18.028 | 48.983 | 1.00 | 17.52 |
| ATOM | 14807 | CA | ILE | 2213 | 26.643 | −18.610 | 47.650 | 1.00 | 17.09 |
| ATOM | 14808 | CB | ILE | 2213 | 27.620 | −17.808 | 46.749 | 1.00 | 17.18 |
| ATOM | 14809 | CG2 | ILE | 2213 | 27.019 | −16.453 | 46.418 | 1.00 | 19.75 |
| ATOM | 14810 | CG1 | ILE | 2213 | 28.972 | −17.636 | 47.447 | 1.00 | 20.12 |
| ATOM | 14811 | CD1 | ILE | 2213 | 29.984 | −16.858 | 46.638 | 1.00 | 18.91 |
| ATOM | 14812 | C | ILE | 2213 | 27.113 | −20.053 | 47.763 | 1.00 | 17.08 |
| ATOM | 14813 | O | ILE | 2213 | 27.899 | −20.384 | 48.648 | 1.00 | 17.01 |
| ATOM | 14814 | N | GLU | 2214 | 26.623 | −20.908 | 46.868 | 1.00 | 17.34 |
| ATOM | 14815 | CA | GLU | 2214 | 26.960 | −22.331 | 46.885 | 1.00 | 18.18 |
| ATOM | 14816 | CB | GLU | 2214 | 25.729 | −23.170 | 46.518 | 1.00 | 19.12 |
| ATOM | 14817 | CG | GLU | 2214 | 24.448 | −22.784 | 47.239 | 1.00 | 20.19 |
| ATOM | 14818 | CD | GLU | 2214 | 23.272 | −23.676 | 46.855 | 1.00 | 23.23 |
| ATOM | 14819 | OE1 | GLU | 2214 | 23.371 | −24.392 | 45.835 | 1.00 | 22.32 |
| ATOM | 14820 | OE2 | GLU | 2214 | 22.243 | −23.657 | 47.563 | 1.00 | 22.19 |
| ATOM | 14821 | C | GLU | 2214 | 28.086 | −22.665 | 45.913 | 1.00 | 18.52 |
| ATOM | 14822 | O | GLU | 2214 | 28.089 | −22.200 | 44.775 | 1.00 | 17.83 |
| ATOM | 14823 | N | GLY | 2215 | 29.048 | −23.467 | 46.361 | 1.00 | 19.25 |
| ATOM | 14824 | CA | GLY | 2215 | 30.148 | −23.837 | 45.484 | 1.00 | 21.18 |
| ATOM | 14825 | C | GLY | 2215 | 31.491 | −23.915 | 46.182 | 1.00 | 21.73 |
| ATOM | 14826 | O | GLY | 2215 | 31.679 | −23.332 | 47.249 | 1.00 | 20.66 |
| ATOM | 14827 | N | GLY | 2216 | 32.434 | −24.629 | 45.575 | 1.00 | 22.21 |
| ATOM | 14828 | CA | GLY | 2216 | 33.749 | −24.765 | 46.176 | 1.00 | 21.89 |
| ATOM | 14829 | C | GLY | 2216 | 34.841 | −23.888 | 45.590 | 1.00 | 21.57 |
| ATOM | 14830 | O | GLY | 2216 | 34.682 | −22.676 | 45.445 | 1.00 | 22.59 |
| ATOM | 14831 | N | GLU | 2217 | 35.962 | −24.524 | 45.266 | 1.00 | 22.40 |
| ATOM | 14832 | CA | GLU | 2217 | 37.140 | −23.869 | 44.701 | 1.00 | 23.58 |
| ATOM | 14833 | CB | GLU | 2217 | 38.119 | −24.934 | 44.188 | 1.00 | 26.77 |
| ATOM | 14834 | CG | GLU | 2217 | 39.116 | −25.430 | 45.213 | 1.00 | 34.27 |
| ATOM | 14835 | CD | GLU | 2217 | 40.230 | −24.431 | 45.458 | 1.00 | 37.56 |
| ATOM | 14836 | OE1 | GLU | 2217 | 39.934 | −23.296 | 45.880 | 1.00 | 41.29 |
| ATOM | 14837 | OE2 | GLU | 2217 | 41.405 | −24.777 | 45.223 | 1.00 | 40.20 |
| ATOM | 14838 | C | GLU | 2217 | 36.881 | −22.869 | 43.584 | 1.00 | 20.77 |
| ATOM | 14839 | O | GLU | 2217 | 37.519 | −21.817 | 43.531 | 1.00 | 19.61 |
| ATOM | 14840 | N | TRP | 2218 | 35.950 | −23.185 | 42.690 | 1.00 | 19.83 |
| ATOM | 14841 | CA | TRP | 2218 | 35.678 | −22.291 | 41.576 | 1.00 | 18.55 |
| ATOM | 14842 | CB | TRP | 2218 | 34.673 | −22.922 | 40.603 | 1.00 | 20.02 |
| ATOM | 14843 | CG | TRP | 2218 | 33.243 | −22.946 | 41.065 | 1.00 | 18.78 |
| ATOM | 14844 | CD2 | TRP | 2218 | 32.217 | −22.023 | 40.687 | 1.00 | 17.40 |
| ATOM | 14845 | CE2 | TRP | 2218 | 31.023 | −22.440 | 41.318 | 1.00 | 16.35 |
| ATOM | 14846 | CE3 | TRP | 2218 | 32.191 | −20.881 | 39.875 | 1.00 | 15.52 |
| ATOM | 14847 | CD1 | TRP | 2218 | 32.654 | −23.860 | 41.889 | 1.00 | 19.14 |
| ATOM | 14848 | NE1 | TRP | 2218 | 31.315 | −23.566 | 42.044 | 1.00 | 19.28 |
| ATOM | 14849 | CZ2 | TRP | 2218 | 29.812 | −21.753 | 41.161 | 1.00 | 17.53 |
| ATOM | 14850 | CZ3 | TRP | 2218 | 30.985 | −20.199 | 39.719 | 1.00 | 17.03 |
| ATOM | 14851 | CH2 | TRP | 2218 | 29.812 | −20.640 | 40.362 | 1.00 | 15.23 |
| ATOM | 14852 | C | TRP | 2218 | 35.204 | −20.906 | 42.006 | 1.00 | 17.22 |
| ATOM | 14853 | O | TRP | 2218 | 35.307 | −19.950 | 41.237 | 1.00 | 17.68 |
| ATOM | 14854 | N | LEU | 2219 | 34.703 | −20.792 | 43.233 | 1.00 | 17.71 |
| ATOM | 14855 | CA | LEU | 2219 | 34.213 | −19.509 | 43.751 | 1.00 | 18.36 |
| ATOM | 14856 | CB | LEU | 2219 | 33.130 | −19.746 | 44.809 | 1.00 | 18.12 |
| ATOM | 14857 | CG | LEU | 2219 | 31.749 | −20.160 | 44.306 | 1.00 | 21.23 |
| ATOM | 14858 | CD1 | LEU | 2219 | 30.838 | −20.464 | 45.498 | 1.00 | 21.25 |
| ATOM | 14859 | CD2 | LEU | 2219 | 31.158 | −19.035 | 43.455 | 1.00 | 19.14 |
| ATOM | 14860 | C | LEU | 2219 | 35.299 | −18.616 | 44.350 | 1.00 | 19.10 |
| ATOM | 14861 | O | LEU | 2219 | 35.067 | −17.432 | 44.601 | 1.00 | 16.47 |
| ATOM | 14862 | N | VAL | 2220 | 36.482 | −19.178 | 44.577 | 1.00 | 18.67 |
| ATOM | 14863 | CA | VAL | 2220 | 37.586 | −18.417 | 45.162 | 1.00 | 19.82 |
| ATOM | 14864 | CB | VAL | 2220 | 38.933 | −19.139 | 44.930 | 1.00 | 20.78 |
| ATOM | 14865 | CG1 | VAL | 2220 | 40.091 | −18.231 | 45.324 | 1.00 | 21.55 |
| ATOM | 14866 | CG2 | VAL | 2220 | 38.966 | −20.415 | 45.742 | 1.00 | 22.40 |
| ATOM | 14867 | C | VAL | 2220 | 37.714 | −16.976 | 44.663 | 1.00 | 18.36 |
| ATOM | 14868 | O | VAL | 2220 | 37.677 | −16.045 | 45.457 | 1.00 | 19.22 |
| ATOM | 14869 | N | GLU | 2221 | 37.864 | −16.797 | 43.353 | 1.00 | 17.93 |
| ATOM | 14870 | CA | GLU | 2221 | 38.010 | −15.462 | 42.790 | 1.00 | 18.36 |
| ATOM | 14871 | CB | GLU | 2221 | 38.208 | −15.543 | 41.281 | 1.00 | 20.61 |
| ATOM | 14872 | CG | GLU | 2221 | 38.200 | −14.198 | 40.587 | 1.00 | 26.76 |
| ATOM | 14873 | CD | GLU | 2221 | 38.529 | −14.303 | 39.113 | 1.00 | 30.40 |
| ATOM | 14874 | OE1 | GLU | 2221 | 37.805 | −15.014 | 38.385 | 1.00 | 30.39 |
| ATOM | 14875 | OE2 | GLU | 2221 | 39.517 | −13.673 | 38.687 | 1.00 | 33.96 |
| ATOM | 14876 | C | GLU | 2221 | 36.811 | −14.567 | 43.091 | 1.00 | 18.56 |
| ATOM | 14877 | O | GLU | 2221 | 36.965 | −13.395 | 43.436 | 1.00 | 16.57 |
| ATOM | 14878 | N | THR | 2222 | 35.617 | −15.126 | 42.946 | 1.00 | 17.43 |
| ATOM | 14879 | CA | THR | 2222 | 34.397 | −14.381 | 43.197 | 1.00 | 17.79 |
| ATOM | 14880 | CB | THR | 2222 | 33.161 | −15.243 | 42.872 | 1.00 | 17.87 |
| ATOM | 14881 | OG1 | THR | 2222 | 33.175 | −15.576 | 41.480 | 1.00 | 19.38 |
| ATOM | 14882 | CG2 | THR | 2222 | 31.882 | −14.485 | 43.188 | 1.00 | 19.18 |
| ATOM | 14883 | C | THR | 2222 | 34.326 | −13.895 | 44.645 | 1.00 | 16.86 |
| ATOM | 14884 | O | THR | 2222 | 33.974 | −12.741 | 44.904 | 1.00 | 15.36 |
| ATOM | 14885 | N | VAL | 2223 | 34.681 | −14.767 | 45.586 | 1.00 | 17.57 |
| ATOM | 14886 | CA | VAL | 2223 | 34.654 | −14.412 | 47.003 | 1.00 | 17.48 |
| ATOM | 14887 | CB | VAL | 2223 | 34.958 | −15.631 | 47.898 | 1.00 | 18.55 |
| ATOM | 14888 | CG1 | VAL | 2223 | 34.953 | −15.211 | 49.373 | 1.00 | 20.37 |
| ATOM | 14889 | CG2 | VAL | 2223 | 33.932 | −16.721 | 47.652 | 1.00 | 16.46 |
| ATOM | 14890 | C | VAL | 2223 | 35.666 | −13.320 | 47.329 | 1.00 | 19.79 |
| ATOM | 14891 | O | VAL | 2223 | 35.352 | −12.349 | 48.024 | 1.00 | 17.16 |
| ATOM | 14892 | N | GLN | 2224 | 36.885 | −13.489 | 46.827 | 1.00 | 18.72 |
| ATOM | 14893 | CA | GLN | 2224 | 37.940 | −12.515 | 47.065 | 1.00 | 21.39 |
| ATOM | 14894 | CB | GLN | 2224 | 39.233 | −12.925 | 46.346 | 1.00 | 24.26 |
| ATOM | 14895 | CG | GLN | 2224 | 39.725 | −14.335 | 46.648 | 1.00 | 29.53 |
| ATOM | 14896 | CD | GLN | 2224 | 41.050 | −14.655 | 45.953 | 1.00 | 33.18 |
| ATOM | 14897 | OE1 | GLN | 2224 | 41.196 | −14.464 | 44.738 | 1.00 | 33.20 |
| ATOM | 14898 | NE2 | GLN | 2224 | 42.018 | −15.150 | 46.722 | 1.00 | 32.66 |
| ATOM | 14899 | C | GLN | 2224 | 37.502 | −11.148 | 46.554 | 1.00 | 19.64 |
| ATOM | 14900 | O | GLN | 2224 | 37.628 | −10.149 | 47.253 | 1.00 | 19.34 |
| ATOM | 14901 | N | MET | 2225 | 36.978 | −11.108 | 45.335 | 1.00 | 19.03 |
| ATOM | 14902 | CA | MET | 2225 | 36.552 | −9.846 | 44.744 | 1.00 | 18.40 |
| ATOM | 14903 | CB | MET | 2225 | 36.306 | −10.026 | 43.249 | 1.00 | 18.27 |
| ATOM | 14904 | CG | MET | 2225 | 37.591 | −10.324 | 42.483 | 1.00 | 22.00 |
| ATOM | 14905 | SD | MET | 2225 | 37.353 | −10.429 | 40.706 | 1.00 | 21.95 |
| ATOM | 14906 | CE | MET | 2225 | 37.447 | −8.712 | 40.259 | 1.00 | 20.84 |
| ATOM | 14907 | C | MET | 2225 | 35.334 | −9.226 | 45.415 | 1.00 | 17.69 |
| ATOM | 14908 | O | MET | 2225 | 35.232 | −8.003 | 45.519 | 1.00 | 16.95 |
| ATOM | 14909 | N | LEU | 2226 | 34.411 | −10.063 | 45.866 | 1.00 | 17.48 |
| ATOM | 14910 | CA | LEU | 2226 | 33.228 | −9.557 | 46.547 | 1.00 | 17.32 |
| ATOM | 14911 | CB | LEU | 2226 | 32.245 | −10.689 | 46.819 | 1.00 | 16.18 |
| ATOM | 14912 | CG | LEU | 2226 | 31.283 | −11.057 | 45.681 | 1.00 | 14.70 |
| ATOM | 14913 | CD1 | LEU | 2226 | 30.554 | −12.330 | 46.056 | 1.00 | 10.61 |
| ATOM | 14914 | CD2 | LEU | 2226 | 30.287 | −9.923 | 45.438 | 1.00 | 15.10 |
| ATOM | 14915 | C | LEU | 2226 | 33.622 | −8.897 | 47.864 | 1.00 | 17.88 |
| ATOM | 14916 | O | LEU | 2226 | 33.203 | −7.777 | 48.158 | 1.00 | 16.44 |
| ATOM | 14917 | N | THR | 2227 | 34.429 | −9.601 | 48.650 | 1.00 | 20.52 |
| ATOM | 14918 | CA | THR | 2227 | 34.889 | −9.099 | 49.946 | 1.00 | 24.25 |
| ATOM | 14919 | CB | THR | 2227 | 35.886 | −10.078 | 50.593 | 1.00 | 25.62 |
| ATOM | 14920 | OG1 | THR | 2227 | 35.234 | −11.334 | 50.830 | 1.00 | 29.56 |
| ATOM | 14921 | CG2 | THR | 2227 | 36.391 | −9.524 | 51.925 | 1.00 | 31.43 |
| ATOM | 14922 | C | THR | 2227 | 35.543 | −7.721 | 49.851 | 1.00 | 24.33 |
| ATOM | 14923 | O | THR | 2227 | 35.235 | −6.825 | 50.643 | 1.00 | 25.20 |
| ATOM | 14924 | N | GLU | 2228 | 36.447 | −7.546 | 48.892 | 1.00 | 25.76 |
| ATOM | 14925 | CA | GLU | 2228 | 37.114 | −6.259 | 48.737 | 1.00 | 26.35 |
| ATOM | 14926 | CB | GLU | 2228 | 38.353 | −6.391 | 47.838 | 1.00 | 30.63 |
| ATOM | 14927 | CG | GLU | 2228 | 38.203 | −7.358 | 46.687 | 1.00 | 31.85 |
| ATOM | 14928 | CD | GLU | 2228 | 39.465 | −7.474 | 45.846 | 1.00 | 33.25 |
| ATOM | 14929 | OE1 | GLU | 2228 | 40.557 | −7.670 | 46.422 | 1.00 | 35.06 |
| ATOM | 14930 | OE2 | GLU | 2228 | 39.368 | −7.385 | 44.607 | 1.00 | 28.52 |
| ATOM | 14931 | C | GLU | 2228 | 36.160 | −5.202 | 48.190 | 1.00 | 26.51 |
| ATOM | 14932 | O | GLU | 2228 | 36.475 | −4.013 | 48.176 | 1.00 | 26.37 |
| ATOM | 14933 | N | ARG | 2229 | 34.983 | −5.632 | 47.747 | 1.00 | 23.68 |
| ATOM | 14934 | CA | ARG | 2229 | 34.004 | −4.690 | 47.228 | 1.00 | 22.45 |
| ATOM | 14935 | CB | ARG | 2229 | 33.456 | −5.199 | 45.893 | 1.00 | 21.74 |
| ATOM | 14936 | CG | ARG | 2229 | 34.481 | −5.021 | 44.779 | 1.00 | 21.55 |
| ATOM | 14937 | CD | ARG | 2229 | 34.202 | −5.839 | 43.529 | 1.00 | 19.25 |
| ATOM | 14938 | NE | ARG | 2229 | 35.257 | −5.628 | 42.538 | 1.00 | 16.72 |
| ATOM | 14939 | CZ | ARG | 2229 | 36.528 | −5.998 | 42.696 | 1.00 | 14.29 |
| ATOM | 14940 | NH1 | ARG | 2229 | 36.918 | −6.607 | 43.804 | 1.00 | 14.68 |
| ATOM | 14941 | NH2 | ARG | 2229 | 37.421 | −5.745 | 41.748 | 1.00 | 16.15 |
| ATOM | 14942 | C | ARG | 2229 | 32.894 | −4.421 | 48.244 | 1.00 | 20.95 |
| ATOM | 14943 | O | ARG | 2229 | 31.753 | −4.099 | 47.889 | 1.00 | 21.04 |
| ATOM | 14944 | N | ALA | 2230 | 33.254 | −4.570 | 49.517 | 1.00 | 19.92 |
| ATOM | 14945 | CA | ALA | 2230 | 32.362 | −4.309 | 50.646 | 1.00 | 17.61 |
| ATOM | 14946 | CB | ALA | 2230 | 31.750 | −2.915 | 50.500 | 1.00 | 20.44 |
| ATOM | 14947 | C | ALA | 2230 | 31.265 | −5.336 | 50.912 | 1.00 | 18.57 |
| ATOM | 14948 | O | ALA | 2230 | 30.374 | −5.085 | 51.722 | 1.00 | 17.70 |
| ATOM | 14949 | N | VAL | 2231 | 31.335 | −6.493 | 50.254 | 1.00 | 17.15 |
| ATOM | 14950 | CA | VAL | 2231 | 30.324 | −7.534 | 50.444 | 1.00 | 17.76 |
| ATOM | 14951 | CB | VAL | 2231 | 29.729 | −8.000 | 49.083 | 1.00 | 17.93 |
| ATOM | 14952 | CG1 | VAL | 2231 | 28.669 | −9.075 | 49.316 | 1.00 | 17.35 |
| ATOM | 14953 | CG2 | VAL | 2231 | 29.125 | −6.822 | 48.335 | 1.00 | 17.32 |
| ATOM | 14954 | C | VAL | 2231 | 30.824 | −8.783 | 51.183 | 1.00 | 17.83 |
| ATOM | 14955 | O | VAL | 2231 | 31.576 | −9.582 | 50.623 | 1.00 | 18.81 |
| ATOM | 14956 | N | PRO | 2232 | 30.412 | −8.969 | 52.450 | 1.00 | 16.77 |
| ATOM | 14957 | CD | PRO | 2232 | 29.701 | −8.061 | 53.370 | 1.00 | 17.78 |
| ATOM | 14958 | CA | PRO | 2232 | 30.884 | −10.168 | 53.145 | 1.00 | 16.29 |
| ATOM | 14959 | CB | PRO | 2232 | 30.523 | −9.898 | 54.608 | 1.00 | 18.07 |
| ATOM | 14960 | CG | PRO | 2232 | 29.390 | −8.958 | 54.529 | 1.00 | 18.75 |
| ATOM | 14961 | C | PRO | 2232 | 30.191 | −11.391 | 52.557 | 1.00 | 15.35 |
| ATOM | 14962 | O | PRO | 2232 | 29.048 | −11.309 | 52.093 | 1.00 | 15.40 |
| ATOM | 14963 | N | VAL | 2233 | 30.887 | −12.520 | 52.576 | 1.00 | 16.70 |
| ATOM | 14964 | CA | VAL | 2233 | 30.365 | −13.753 | 51.995 | 1.00 | 15.99 |
| ATOM | 14965 | CB | VAL | 2233 | 31.225 | −14.174 | 50.772 | 1.00 | 17.33 |
| ATOM | 14966 | CG1 | VAL | 2233 | 30.743 | −15.516 | 50.231 | 1.00 | 15.74 |
| ATOM | 14967 | CG2 | VAL | 2233 | 31.189 | −13.105 | 49.708 | 1.00 | 16.38 |
| ATOM | 14968 | C | VAL | 2233 | 30.316 | −14.958 | 52.924 | 1.00 | 16.80 |
| ATOM | 14969 | O | VAL | 2233 | 31.250 | −15.224 | 53.681 | 1.00 | 16.97 |
| ATOM | 14970 | N | CYS | 2234 | 29.217 | −15.695 | 52.838 | 1.00 | 15.58 |
| ATOM | 14971 | CA | CYS | 2234 | 29.055 | −16.917 | 53.599 | 1.00 | 16.50 |
| ATOM | 14972 | CB | CYS | 2234 | 27.683 | −16.974 | 54.281 | 1.00 | 15.73 |
| ATOM | 14973 | SG | CYS | 2234 | 27.369 | −18.518 | 55.159 | 1.00 | 16.61 |
| ATOM | 14974 | C | CYS | 2234 | 29.140 | −18.027 | 52.552 | 1.00 | 18.28 |
| ATOM | 14975 | O | CYS | 2234 | 28.428 | −17.992 | 51.544 | 1.00 | 19.80 |
| ATOM | 14976 | N | GLY | 2235 | 30.027 | −18.990 | 52.775 | 1.00 | 17.80 |
| ATOM | 14977 | CA | GLY | 2235 | 30.162 | −20.093 | 51.841 | 1.00 | 17.79 |
| ATOM | 14978 | C | GLY | 2235 | 29.081 | −21.125 | 52.090 | 1.00 | 16.39 |
| ATOM | 14979 | O | GLY | 2235 | 28.330 | −21.021 | 53.060 | 1.00 | 16.55 |
| ATOM | 14980 | N | HIS | 2236 | 28.989 | −22.123 | 51.219 | 1.00 | 16.49 |
| ATOM | 14981 | CA | HIS | 2236 | 27.981 | −23.169 | 51.378 | 1.00 | 19.27 |
| ATOM | 14982 | CB | HIS | 2236 | 26.646 | −22.690 | 50.790 | 1.00 | 19.17 |
| ATOM | 14983 | CG | HIS | 2236 | 25.489 | −23.600 | 51.058 | 1.00 | 20.45 |
| ATOM | 14984 | CD2 | HIS | 2236 | 25.434 | −24.925 | 51.334 | 1.00 | 21.15 |
| ATOM | 14985 | ND1 | HIS | 2236 | 24.182 | −23.163 | 51.018 | 1.00 | 20.57 |
| ATOM | 14986 | CE1 | HIS | 2236 | 23.372 | −24.178 | 51.258 | 1.00 | 20.23 |
| ATOM | 14987 | NE2 | HIS | 2236 | 24.107 | −25.260 | 51.454 | 1.00 | 19.86 |
| ATOM | 14988 | C | HIS | 2236 | 28.480 | −24.420 | 50.667 | 1.00 | 19.78 |
| ATOM | 14989 | O | HIS | 2236 | 28.538 | −24.463 | 49.438 | 1.00 | 18.95 |
| ATOM | 14990 | N | LEU | 2237 | 28.849 | −25.427 | 51.455 | 1.00 | 21.72 |
| ATOM | 14991 | CA | LEU | 2237 | 29.375 | −26.686 | 50.926 | 1.00 | 22.90 |
| ATOM | 14992 | CB | LEU | 2237 | 30.823 | −26.880 | 51.383 | 1.00 | 21.03 |
| ATOM | 14993 | CG | LEU | 2237 | 31.850 | −25.837 | 50.920 | 1.00 | 21.00 |
| ATOM | 14994 | CD1 | LEU | 2237 | 33.206 | −26.144 | 51.536 | 1.00 | 20.20 |
| ATOM | 14995 | CD2 | LEU | 2237 | 31.942 | −25.844 | 49.401 | 1.00 | 22.91 |
| ATOM | 14996 | C | LEU | 2237 | 28.542 | −27.881 | 51.365 | 1.00 | 22.62 |
| ATOM | 14997 | O | LEU | 2237 | 27.701 | −27.768 | 52.256 | 1.00 | 22.68 |
| ATOM | 14998 | N | GLY | 2238 | 28.793 | −29.028 | 50.739 | 1.00 | 23.03 |
| ATOM | 14999 | CA | GLY | 2238 | 28.052 | −30.235 | 51.061 | 1.00 | 22.71 |
| ATOM | 15000 | C | GLY | 2238 | 26.899 | −30.398 | 50.096 | 1.00 | 24.92 |
| ATOM | 15001 | O | GLY | 2238 | 27.094 | −30.367 | 48.880 | 1.00 | 24.62 |
| ATOM | 15002 | N | LEU | 2239 | 25.694 | −30.565 | 50.631 | 1.00 | 25.75 |
| ATOM | 15003 | CA | LEU | 2239 | 24.506 | −30.715 | 49.801 | 1.00 | 27.14 |
| ATOM | 15004 | CB | LEU | 2239 | 23.427 | −31.485 | 50.573 | 1.00 | 27.64 |
| ATOM | 15005 | CG | LEU | 2239 | 22.208 | −32.031 | 49.822 | 1.00 | 29.36 |
| ATOM | 15006 | CD1 | LEU | 2239 | 21.373 | −32.870 | 50.782 | 1.00 | 29.18 |
| ATOM | 15007 | CD2 | LEU | 2239 | 21.380 | −30.905 | 49.248 | 1.00 | 29.54 |
| ATOM | 15008 | C | LEU | 2239 | 23.996 | −29.323 | 49.416 | 1.00 | 27.73 |
| ATOM | 15009 | O | LEU | 2239 | 23.270 | −28.681 | 50.181 | 1.00 | 29.81 |
| ATOM | 15010 | N | THR | 2240 | 24.391 | −28.866 | 48.231 | 1.00 | 24.58 |
| ATOM | 15011 | CA | THR | 2240 | 23.998 | −27.559 | 47.714 | 1.00 | 24.57 |
| ATOM | 15012 | CD | THR | 2240 | 25.156 | −26.932 | 46.894 | 1.00 | 25.13 |
| ATOM | 15013 | OG1 | THR | 2240 | 25.600 | −27.858 | 45.890 | 1.00 | 23.49 |
| ATOM | 15014 | CG2 | THR | 2240 | 26.336 | −26.607 | 47.810 | 1.00 | 26.36 |
| ATOM | 15015 | C | THR | 2240 | 22.751 | −27.721 | 46.840 | 1.00 | 24.62 |
| ATOM | 15016 | O | THR | 2240 | 22.843 | −28.055 | 45.661 | 1.00 | 23.83 |
| ATOM | 15017 | N | PRO | 2241 | 21.565 | −27.473 | 47.420 | 1.00 | 24.68 |
| ATOM | 15018 | CD | PRO | 2241 | 21.400 | −26.858 | 48.749 | 1.00 | 25.30 |
| ATOM | 15019 | CA | PRO | 2241 | 20.267 | −27.592 | 46.741 | 1.00 | 24.86 |
| ATOM | 15020 | CB | PRO | 2241 | 19.283 | −27.004 | 47.758 | 1.00 | 24.84 |
| ATOM | 15021 | CG | PRO | 2241 | 20.136 | −26.080 | 48.575 | 1.00 | 27.84 |
| ATOM | 15022 | C | PRO | 2241 | 20.112 | −27.002 | 45.337 | 1.00 | 24.57 |
| ATOM | 15023 | O | PRO | 2241 | 19.338 | −27.530 | 44.534 | 1.00 | 24.58 |
| ATOM | 15024 | N | GLN | 2242 | 20.828 | −25.924 | 45.026 | 1.00 | 22.22 |
| ATOM | 15025 | CA | GLN | 2242 | 20.737 | −25.335 | 43.687 | 1.00 | 22.19 |
| ATOM | 15026 | CB | GLN | 2242 | 21.591 | −24.064 | 43.599 | 1.00 | 21.42 |
| ATOM | 15027 | CG | GLN | 2242 | 20.849 | −22.785 | 43.987 | 1.00 | 19.29 |
| ATOM | 15028 | CD | GLN | 2242 | 21.784 | −21.601 | 44.191 | 1.00 | 20.80 |
| ATOM | 15029 | OE1 | GLN | 2242 | 22.795 | −21.460 | 43.498 | 1.00 | 18.83 |
| ATOM | 15030 | NE2 | GLN | 2242 | 21.441 | −20.736 | 45.140 | 1.00 | 17.73 |
| ATOM | 15031 | C | GLN | 2242 | 21.169 | −26.331 | 42.602 | 1.00 | 21.96 |
| ATOM | 15032 | O | GLN | 2242 | 20.690 | −26.261 | 41.470 | 1.00 | 20.71 |
| ATOM | 15033 | N | SER | 2243 | 22.057 | −27.259 | 42.962 | 1.00 | 20.88 |
| ATOM | 15034 | CA | SER | 2243 | 22.553 | −28.271 | 42.032 | 1.00 | 20.30 |
| ATOM | 15035 | CB | SER | 2243 | 24.043 | −28.559 | 42.293 | 1.00 | 20.51 |
| ATOM | 15036 | OG | SER | 2243 | 24.851 | −27.405 | 42.099 | 1.00 | 18.03 |
| ATOM | 15037 | C | SER | 2243 | 21.772 | −29.587 | 42.140 | 1.00 | 20.10 |
| ATOM | 15038 | O | SER | 2243 | 22.281 | −30.647 | 41.764 | 1.00 | 19.42 |
| ATOM | 15039 | N | VAL | 2244 | 20.538 | −29.519 | 42.633 | 1.00 | 20.27 |
| ATOM | 15040 | CA | VAL | 2244 | 19.710 | −30.712 | 42.806 | 1.00 | 19.69 |
| ATOM | 15041 | CB | VAL | 2244 | 18.285 | −30.340 | 43.313 | 1.00 | 20.85 |
| ATOM | 15042 | CG1 | VAL | 2244 | 17.525 | −29.559 | 42.245 | 1.00 | 19.91 |
| ATOM | 15043 | CG2 | VAL | 2244 | 17.525 | −31.601 | 43.698 | 1.00 | 21.83 |
| ATOM | 15044 | C | VAL | 2244 | 19.581 | −31.558 | 41.538 | 1.00 | 20.01 |
| ATOM | 15045 | O | VAL | 2244 | 19.484 | −32.785 | 41.611 | 1.00 | 19.12 |
| ATOM | 15046 | N | ASN | 2245 | 19.582 | −30.907 | 40.379 | 1.00 | 17.97 |
| ATOM | 15047 | CA | ASN | 2245 | 19.471 | −31.626 | 39.118 | 1.00 | 19.30 |
| ATOM | 15048 | CB | ASN | 2245 | 19.110 | −30.653 | 37.993 | 1.00 | 17.46 |
| ATOM | 15049 | CG | ASN | 2245 | 17.709 | −30.102 | 38.146 | 1.00 | 19.80 |
| ATOM | 15050 | OD1 | ASN | 2245 | 16.727 | −30.850 | 38.058 | 1.00 | 16.40 |
| ATOM | 15051 | ND2 | ASN | 2245 | 17.601 | −28.796 | 38.386 | 1.00 | 18.40 |
| ATOM | 15052 | C | ASN | 2245 | 20.756 | −32.367 | 38.787 | 1.00 | 19.89 |
| ATOM | 15053 | O | ASN | 2245 | 20.734 | −33.401 | 38.114 | 1.00 | 20.19 |
| ATOM | 15054 | N | ILE | 2246 | 21.881 | −31.835 | 39.254 | 1.00 | 20.52 |
| ATOM | 15055 | CA | ILE | 2246 | 23.172 | −32.482 | 39.011 | 1.00 | 21.49 |
| ATOM | 15056 | CB | ILE | 2246 | 24.365 | −31.581 | 39.411 | 1.00 | 20.68 |
| ATOM | 15057 | CG2 | ILE | 2246 | 25.654 | −32.401 | 39.365 | 1.00 | 21.13 |
| ATOM | 15058 | CG1 | ILE | 2246 | 24.464 | −30.365 | 38.472 | 1.00 | 20.19 |
| ATOM | 15059 | CD1 | ILE | 2246 | 25.006 | −30.675 | 37.085 | 1.00 | 18.99 |
| ATOM | 15060 | C | ILE | 2246 | 23.266 | −33.759 | 39.847 | 1.00 | 22.01 |
| ATOM | 15061 | O | ILE | 2246 | 23.619 | −34.821 | 39.333 | 1.00 | 21.51 |
| ATOM | 15062 | N | PHE | 2247 | 22.953 | −33.647 | 41.136 | 1.00 | 22.01 |
| ATOM | 15063 | CA | PHE | 2247 | 23.028 | −34.796 | 42.036 | 1.00 | 24.57 |
| ATOM | 15064 | CB | PHE | 2247 | 22.955 | −34.351 | 43.498 | 1.00 | 27.03 |
| ATOM | 15065 | CG | PHE | 2247 | 23.907 | −33.243 | 43.852 | 1.00 | 30.51 |
| ATOM | 15066 | CD1 | PHE | 2247 | 25.256 | −33.328 | 43.522 | 1.00 | 31.67 |
| ATOM | 15067 | CD2 | PHE | 2247 | 23.449 | −32.113 | 44.530 | 1.00 | 32.37 |
| ATOM | 15068 | CE1 | PHE | 2247 | 26.139 | −32.302 | 43.860 | 1.00 | 33.67 |
| ATOM | 15069 | CE2 | PHE | 2247 | 24.320 | −31.082 | 44.873 | 1.00 | 34.16 |
| ATOM | 15070 | CZ | PHE | 2247 | 25.667 | −31.174 | 44.538 | 1.00 | 34.12 |
| ATOM | 15071 | C | PHE | 2247 | 21.915 | −35.806 | 41.779 | 1.00 | 24.16 |
| ATOM | 15072 | O | PHE | 2247 | 22.042 | −36.975 | 42.136 | 1.00 | 25.60 |
| ATOM | 15073 | N | GLY | 2248 | 20.828 | −35.349 | 41.166 | 1.00 | 22.84 |
| ATOM | 15074 | CA | GLY | 2248 | 19.712 | −36.233 | 40.893 | 1.00 | 23.98 |
| ATOM | 15075 | C | GLY | 2248 | 18.818 | −36.384 | 42.107 | 1.00 | 25.53 |
| ATOM | 15076 | O | GLY | 2248 | 18.079 | −37.362 | 42.238 | 1.00 | 25.77 |
| ATOM | 15077 | N | GLY | 2249 | 18.884 | −35.405 | 43.002 | 1.00 | 26.74 |
| ATOM | 15078 | CA | GLY | 2249 | 18.081 | −35.442 | 44.209 | 1.00 | 29.60 |
| ATOM | 15079 | C | GLY | 2249 | 18.844 | −34.872 | 45.387 | 1.00 | 31.46 |
| ATOM | 15080 | O | GLY | 2249 | 19.994 | −34.452 | 45.241 | 1.00 | 31.11 |
| ATOM | 15081 | N | TYR | 2250 | 18.207 | −34.848 | 46.553 | 1.00 | 33.81 |
| ATOM | 15082 | CA | TYR | 2250 | 18.843 | −34.330 | 47.758 | 1.00 | 36.66 |
| ATOM | 15083 | CB | TYR | 2250 | 17.819 | −33.608 | 48.638 | 1.00 | 38.09 |
| ATOM | 15084 | CG | TYR | 2250 | 17.136 | −32.453 | 47.945 | 1.00 | 40.93 |
| ATOM | 15085 | CD1 | TYR | 2250 | 15.946 | −32.640 | 47.243 | 1.00 | 41.37 |
| ATOM | 15086 | CE1 | TYR | 2250 | 15.325 | −31.580 | 46.584 | 1.00 | 43.82 |
| ATOM | 15087 | CD2 | TYR | 2250 | 17.694 | −31.174 | 47.970 | 1.00 | 41.83 |
| ATOM | 15088 | CE2 | TYR | 2250 | 17.086 | −30.108 | 47.314 | 1.00 | 43.92 |
| ATOM | 15089 | CZ | TYR | 2250 | 15.902 | −30.317 | 46.624 | 1.00 | 43.98 |
| ATOM | 15090 | OH | TYR | 2250 | 15.303 | −29.264 | 45.971 | 1.00 | 45.48 |
| ATOM | 15091 | C | TYR | 2250 | 19.484 | −35.477 | 48.528 | 1.00 | 37.32 |
| ATOM | 15092 | O | TYR | 2250 | 18.859 | −36.090 | 49.392 | 1.00 | 38.37 |
| ATOM | 15093 | N | LYS | 2251 | 20.741 | −35.755 | 48.204 | 1.00 | 37.64 |
| ATOM | 15094 | CA | LYS | 2251 | 21.481 | −36.839 | 48.830 | 1.00 | 37.41 |
| ATOM | 15095 | CB | LYS | 2251 | 22.013 | −37.776 | 47.742 | 1.00 | 37.65 |
| ATOM | 15096 | CG | LYS | 2251 | 20.954 | −38.168 | 46.711 | 1.00 | 37.38 |
| ATOM | 15097 | CD | LYS | 2251 | 21.580 | −38.742 | 45.448 | 1.00 | 37.94 |
| ATOM | 15098 | CE | LYS | 2251 | 20.519 | −39.094 | 44.415 | 1.00 | 37.48 |
| ATOM | 15099 | NZ | LYS | 2251 | 21.114 | −39.517 | 43.119 | 1.00 | 33.97 |
| ATOM | 15100 | C | LYS | 2251 | 22.638 | −36.287 | 49.659 | 1.00 | 37.97 |
| ATOM | 15101 | O | LYS | 2251 | 23.176 | −35.221 | 49.358 | 1.00 | 36.96 |
| ATOM | 15102 | N | VAL | 2252 | 23.012 | −37.018 | 50.706 | 1.00 | 37.82 |
| ATOM | 15103 | CA | VAL | 2252 | 24.106 | −36.605 | 51.576 | 1.00 | 39.23 |
| ATOM | 15104 | CB | VAL | 2252 | 24.319 | −37.621 | 52.717 | 1.00 | 38.53 |
| ATOM | 15105 | CG1 | VAL | 2252 | 25.416 | −37.137 | 53.651 | 1.00 | 39.17 |
| ATOM | 15106 | CG2 | VAL | 2252 | 23.024 | −37.821 | 53.479 | 1.00 | 39.33 |
| ATOM | 15107 | C | VAL | 2252 | 25.390 | −36.497 | 50.759 | 1.00 | 39.59 |
| ATOM | 15108 | O | VAL | 2252 | 25.584 | −37.240 | 49.798 | 1.00 | 39.94 |
| ATOM | 15109 | N | GLN | 2253 | 26.265 | −35.574 | 51.144 | 1.00 | 40.19 |
| ATOM | 15110 | CA | GLN | 2253 | 27.520 | −35.376 | 50.430 | 1.00 | 41.92 |
| ATOM | 15111 | CB | GLN | 2253 | 27.512 | −34.008 | 49.743 | 1.00 | 42.02 |
| ATOM | 15112 | CG | GLN | 2253 | 28.197 | −33.976 | 48.384 | 1.00 | 43.44 |
| ATOM | 15113 | CD | GLN | 2253 | 27.479 | −34.825 | 47.349 | 1.00 | 42.91 |
| ATOM | 15114 | OE1 | GLN | 2253 | 26.275 | −34.672 | 47.128 | 1.00 | 43.91 |
| ATOM | 15115 | NE2 | GLN | 2253 | 28.216 | −35.723 | 46.704 | 1.00 | 44.45 |
| ATOM | 15116 | C | GLN | 2253 | 28.697 | −35.469 | 51.398 | 1.00 | 42.35 |
| ATOM | 15117 | O | GLN | 2253 | 28.522 | −35.358 | 52.610 | 1.00 | 42.17 |
| ATOM | 15118 | N | GLY | 2254 | 29.895 | −35.677 | 50.862 | 1.00 | 43.70 |
| ATOM | 15119 | CA | GLY | 2254 | 31.069 | −35.779 | 51.709 | 1.00 | 45.07 |
| ATOM | 15120 | C | GLY | 2254 | 31.432 | −37.209 | 52.071 | 1.00 | 46.95 |
| ATOM | 15121 | O | GLY | 2254 | 32.521 | −37.466 | 52.581 | 1.00 | 47.08 |
| ATOM | 15122 | N | ARG | 2255 | 30.517 | −38.140 | 51.814 | 1.00 | 48.47 |
| ATOM | 15123 | CA | ARG | 2255 | 30.742 | −39.552 | 52.105 | 1.00 | 49.93 |
| ATOM | 15124 | CB | ARG | 2255 | 29.517 | −40.378 | 51.696 | 1.00 | 50.98 |
| ATOM | 15125 | CG | ARG | 2255 | 28.224 | −40.026 | 52.424 | 1.00 | 51.85 |
| ATOM | 15126 | CD | ARG | 2255 | 28.282 | −40.425 | 53.887 | 1.00 | 52.47 |
| ATOM | 15127 | NE | ARG | 2255 | 27.032 | −40.148 | 54.596 | 1.00 | 53.65 |
| ATOM | 15128 | CZ | ARG | 2255 | 25.864 | −40.724 | 54.322 | 1.00 | 52.93 |
| ATOM | 15129 | NH1 | ARG | 2255 | 25.768 | −41.618 | 53.349 | 1.00 | 52.83 |
| ATOM | 15130 | NH2 | ARG | 2255 | 24.786 | −40.409 | 55.027 | 1.00 | 53.44 |
| ATOM | 15131 | C | ARG | 2255 | 31.966 | −40.058 | 51.342 | 1.00 | 50.92 |
| ATOM | 15132 | O | ARG | 2255 | 31.965 | −40.097 | 50.111 | 1.00 | 50.96 |
| ATOM | 15133 | N | GLY | 2256 | 33.010 | −40.444 | 52.070 | 1.00 | 51.41 |
| ATOM | 15134 | CA | GLY | 2256 | 34.211 | −40.938 | 51.419 | 1.00 | 52.44 |
| ATOM | 15135 | C | GLY | 2256 | 35.428 | −40.069 | 51.677 | 1.00 | 52.97 |
| ATOM | 15136 | O | GLY | 2256 | 35.313 | −38.854 | 51.819 | 1.00 | 52.71 |
| ATOM | 15137 | N | ASP | 2257 | 36.599 | −40.694 | 51.732 | 1.00 | 53.48 |
| ATOM | 15138 | CA | ASP | 2257 | 37.838 | −39.969 | 51.982 | 1.00 | 53.79 |
| ATOM | 15139 | CB | ASP | 2257 | 39.023 | −40.934 | 52.009 | 1.00 | 55.06 |
| ATOM | 15140 | CG | ASP | 2257 | 38.901 | −41.970 | 53.105 | 1.00 | 56.30 |
| ATOM | 15141 | OD1 | ASP | 2257 | 38.722 | −41.576 | 54.277 | 1.00 | 56.85 |
| ATOM | 15142 | OD2 | ASP | 2257 | 38.987 | −43.177 | 52.797 | 1.00 | 58.02 |
| ATOM | 15143 | C | ASP | 2257 | 38.084 | −38.893 | 50.935 | 1.00 | 53.11 |
| ATOM | 15144 | O | ASP | 2257 | 38.356 | −37.741 | 51.269 | 1.00 | 53.00 |
| ATOM | 15145 | N | GLU | 2258 | 37.989 | −39.273 | 49.666 | 1.00 | 52.48 |
| ATOM | 15146 | CA | GLU | 2258 | 38.213 | −38.335 | 48.576 | 1.00 | 51.39 |
| ATOM | 15147 | CB | GLU | 2258 | 38.123 | −39.062 | 47.234 | 1.00 | 53.26 |
| ATOM | 15148 | CG | GLU | 2258 | 38.351 | −38.169 | 46.028 | 1.00 | 55.82 |
| ATOM | 15149 | CD | GLU | 2258 | 38.623 | −38.959 | 44.762 | 1.00 | 57.62 |
| ATOM | 15150 | OE1 | GLU | 2258 | 39.678 | −39.629 | 44.695 | 1.00 | 58.68 |
| ATOM | 15151 | OE2 | GLU | 2258 | 37.783 | −38.913 | 43.837 | 1.00 | 58.41 |
| ATOM | 15152 | C | GLU | 2258 | 37.210 | −37.188 | 48.620 | 1.00 | 49.36 |
| ATOM | 15153 | O | GLU | 2258 | 37.591 | −36.020 | 48.567 | 1.00 | 48.74 |
| ATOM | 15154 | N | ALA | 2259 | 35.929 | −37.527 | 48.721 | 1.00 | 47.48 |
| ATOM | 15155 | CA | ALA | 2259 | 34.877 | −36.521 | 48.778 | 1.00 | 45.65 |
| ATOM | 15156 | CB | ALA | 2259 | 33.510 | −37.191 | 48.747 | 1.00 | 45.32 |
| ATOM | 15157 | C | ALA | 2259 | 35.023 | −35.688 | 50.046 | 1.00 | 43.78 |
| ATOM | 15158 | O | ALA | 2259 | 34.742 | −34.491 | 50.047 | 1.00 | 43.87 |
| ATOM | 15159 | N | GLY | 2260 | 35.466 | −36.330 | 51.121 | 1.00 | 42.56 |
| ATOM | 15160 | CA | GLY | 2260 | 35.644 | −35.632 | 52.380 | 1.00 | 41.13 |
| ATOM | 15161 | C | GLY | 2260 | 36.794 | −34.645 | 52.338 | 1.00 | 40.59 |
| ATOM | 15162 | O | GLY | 2260 | 36.680 | −33.524 | 52.837 | 1.00 | 39.79 |
| ATOM | 15163 | N | ASP | 2261 | 37.909 | −35.056 | 51.744 | 1.00 | 38.76 |
| ATOM | 15164 | CA | ASP | 2261 | 39.070 | −34.182 | 51.647 | 1.00 | 38.24 |
| ATOM | 15165 | CB | ASP | 2261 | 40.291 | −34.959 | 51.141 | 1.00 | 39.33 |
| ATOM | 15166 | CG | ASP | 2261 | 40.635 | −36.142 | 52.025 | 1.00 | 40.38 |
| ATOM | 15167 | OD1 | ASP | 2261 | 40.613 | −35.990 | 53.267 | 1.00 | 39.92 |
| ATOM | 15168 | OD2 | ASP | 2261 | 40.938 | −37.224 | 51.477 | 1.00 | 42.70 |
| ATOM | 15169 | C | ASP | 2261 | 38.775 | −33.022 | 50.707 | 1.00 | 36.18 |
| ATOM | 15170 | O | ASP | 2261 | 39.279 | −31.915 | 50.899 | 1.00 | 35.73 |
| ATOM | 15171 | N | GLN | 2262 | 37.951 | −33.279 | 49.695 | 1.00 | 35.55 |
| ATOM | 15172 | CA | GLN | 2262 | 37.588 | −32.246 | 48.728 | 1.00 | 34.77 |
| ATOM | 15173 | CB | GLN | 2262 | 36.718 | −32.844 | 47.617 | 1.00 | 35.12 |
| ATOM | 15174 | CG | GLN | 2262 | 36.351 | −31.864 | 46.504 | 1.00 | 37.06 |
| ATOM | 15175 | CD | GLN | 2262 | 37.568 | −31.284 | 45.803 | 1.00 | 38.05 |
| ATOM | 15176 | OE1 | GLN | 2262 | 38.409 | −32.019 | 45.281 | 1.00 | 39.24 |
| ATOM | 15177 | NE2 | GLN | 2262 | 37.665 | −29.960 | 45.782 | 1.00 | 38.19 |
| ATOM | 15178 | C | GLN | 2262 | 36.839 | −31.112 | 49.422 | 1.00 | 33.47 |
| ATOM | 15179 | O | GLN | 2262 | 37.118 | −29.939 | 49.177 | 1.00 | 33.28 |
| ATOM | 15180 | N | LEU | 2263 | 35.888 | −31.469 | 50.284 | 1.00 | 32.83 |
| ATOM | 15181 | CA | LEU | 2263 | 35.105 | −30.478 | 51.020 | 1.00 | 31.73 |
| ATOM | 15182 | CB | LEU | 2263 | 33.968 | −31.153 | 51.796 | 1.00 | 32.63 |
| ATOM | 15183 | CG | LEU | 2263 | 32.813 | −31.739 | 50.973 | 1.00 | 35.13 |
| ATOM | 15184 | CD1 | LEU | 2263 | 31.733 | −32.282 | 51.905 | 1.00 | 36.24 |
| ATOM | 15185 | CD2 | LEU | 2263 | 32.230 | −30.652 | 50.073 | 1.00 | 35.79 |
| ATOM | 15186 | C | LEU | 2263 | 35.993 | −29.701 | 51.986 | 1.00 | 30.80 |
| ATOM | 15187 | O | LEU | 2263 | 35.874 | −28.484 | 52.110 | 1.00 | 30.26 |
| ATOM | 15188 | N | LEU | 2264 | 36.883 | −30.408 | 52.674 | 1.00 | 29.92 |
| ATOM | 15189 | CA | LEU | 2264 | 37.789 | −29.762 | 53.613 | 1.00 | 28.33 |
| ATOM | 15190 | CB | LEU | 2264 | 38.704 | −30.804 | 54.259 | 1.00 | 31.47 |
| ATOM | 15191 | CG | LEU | 2264 | 39.283 | −30.483 | 55.639 | 1.00 | 33.11 |
| ATOM | 15192 | CD1 | LEU | 2264 | 40.170 | −31.648 | 56.068 | 1.00 | 32.67 |
| ATOM | 15193 | CD2 | LEU | 2264 | 40.069 | −29.188 | 55.623 | 1.00 | 33.38 |
| ATOM | 15194 | C | LEU | 2264 | 38.631 | −28.742 | 52.855 | 1.00 | 26.63 |
| ATOM | 15195 | O | LEU | 2264 | 38.761 | −27.594 | 53.275 | 1.00 | 26.53 |
| ATOM | 15196 | N | SER | 2265 | 39.198 | −29.170 | 51.732 | 1.00 | 25.34 |
| ATOM | 15197 | CA | SER | 2265 | 40.029 | −28.291 | 50.918 | 1.00 | 25.06 |
| ATOM | 15198 | CB | SER | 2265 | 40.546 | −29.036 | 49.686 | 1.00 | 25.94 |
| ATOM | 15199 | OG | SER | 2265 | 41.362 | −28.193 | 48.895 | 1.00 | 26.74 |
| ATOM | 15200 | C | SER | 2265 | 39.237 | −27.067 | 50.475 | 1.00 | 24.21 |
| ATOM | 15201 | O | SER | 2265 | 39.740 | −25.945 | 50.524 | 1.00 | 22.95 |
| ATOM | 15202 | N | ASP | 2266 | 37.998 | −27.296 | 50.044 | 1.00 | 23.92 |
| ATOM | 15203 | CA | ASP | 2266 | 37.124 | −26.215 | 49.594 | 1.00 | 24.88 |
| ATOM | 15204 | CB | ASP | 2266 | 35.805 | −26.776 | 49.045 | 1.00 | 25.99 |
| ATOM | 15205 | CG | ASP | 2266 | 35.959 | −27.401 | 47.671 | 1.00 | 29.42 |
| ATOM | 15206 | OD1 | ASP | 2266 | 36.533 | −26.744 | 46.775 | 1.00 | 31.95 |
| ATOM | 15207 | OD2 | ASP | 2266 | 35.498 | −28.545 | 47.478 | 1.00 | 34.49 |
| ATOM | 15208 | C | ASP | 2266 | 36.827 | −25.231 | 50.724 | 1.00 | 22.16 |
| ATOM | 15209 | O | ASP | 2266 | 36.865 | −24.013 | 50.529 | 1.00 | 22.29 |
| ATOM | 15210 | N | ALA | 2267 | 36.531 | −25.767 | 51.902 | 1.00 | 22.20 |
| ATOM | 15211 | CA | ALA | 2267 | 36.237 | −24.944 | 53.069 | 1.00 | 20.33 |
| ATOM | 15212 | CB | ALA | 2267 | 35.952 | −25.836 | 54.280 | 1.00 | 19.05 |
| ATOM | 15213 | C | ALA | 2267 | 37.419 | −24.021 | 53.354 | 1.00 | 20.70 |
| ATOM | 15214 | O | ALA | 2267 | 37.247 | −22.817 | 53.546 | 1.00 | 18.09 |
| ATOM | 15215 | N | LEU | 2268 | 38.622 | −24.584 | 53.378 | 1.00 | 19.57 |
| ATOM | 15216 | CA | LEU | 2268 | 39.812 | −23.781 | 53.629 | 1.00 | 22.22 |
| ATOM | 15217 | CB | LEU | 2268 | 41.054 | −24.671 | 53.712 | 1.00 | 22.33 |
| ATOM | 15218 | CG | LEU | 2268 | 41.220 | −25.470 | 54.997 | 1.00 | 23.44 |
| ATOM | 15219 | CD1 | LEU | 2268 | 42.400 | −26.428 | 54.850 | 1.00 | 24.11 |
| ATOM | 15220 | CD2 | LEU | 2268 | 41.430 | −24.519 | 56.173 | 1.00 | 23.26 |
| ATOM | 15221 | C | LEU | 2268 | 40.016 | −22.736 | 52.544 | 1.00 | 21.76 |
| ATOM | 15222 | O | LEU | 2268 | 40.387 | −21.600 | 52.837 | 1.00 | 23.46 |
| ATOM | 15223 | N | ALA | 2269 | 39.772 | −23.120 | 51.295 | 1.00 | 21.45 |
| ATOM | 15224 | CA | ALA | 2269 | 39.944 | −22.204 | 50.174 | 1.00 | 22.17 |
| ATOM | 15225 | CB | ALA | 2269 | 39.709 | −22.938 | 48.853 | 1.00 | 22.22 |
| ATOM | 15226 | C | ALA | 2269 | 39.000 | −21.015 | 50.283 | 1.00 | 21.51 |
| ATOM | 15227 | O | ALA | 2269 | 39.399 | −19.871 | 50.061 | 1.00 | 21.07 |
| ATOM | 15228 | N | LEU | 2270 | 37.745 | −21.285 | 50.620 | 1.00 | 19.82 |
| ATOM | 15229 | CA | LEU | 2270 | 36.761 | −20.215 | 50.753 | 1.00 | 21.19 |
| ATOM | 15230 | CB | LEU | 2270 | 35.377 | −20.804 | 51.028 | 1.00 | 19.10 |
| ATOM | 15231 | CG | LEU | 2270 | 34.777 | −21.647 | 49.897 | 1.00 | 20.93 |
| ATOM | 15232 | CD1 | LEU | 2270 | 33.488 | −22.306 | 50.382 | 1.00 | 20.19 |
| ATOM | 15233 | CD2 | LEU | 2270 | 34.509 | −20.763 | 48.682 | 1.00 | 20.41 |
| ATOM | 15234 | C | LEU | 2270 | 37.162 | −19.269 | 51.882 | 1.00 | 20.84 |
| ATOM | 15235 | O | LEU | 2270 | 37.037 | −18.050 | 51.761 | 1.00 | 21.29 |
| ATOM | 15236 | N | GLU | 2271 | 37.644 | −19.834 | 52.982 | 1.00 | 22.99 |
| ATOM | 15237 | CA | GLU | 2271 | 38.072 | −19.022 | 54.111 | 1.00 | 22.49 |
| ATOM | 15238 | CB | GLU | 2271 | 38.484 | −19.919 | 55.277 | 1.00 | 23.48 |
| ATOM | 15239 | CG | GLU | 2271 | 39.110 | −19.168 | 56.431 | 1.00 | 24.86 |
| ATOM | 15240 | CD | GLU | 2271 | 39.540 | −20.086 | 57.555 | 1.00 | 27.13 |
| ATOM | 15241 | OE1 | GLU | 2271 | 40.279 | −21.059 | 57.280 | 1.00 | 26.64 |
| ATOM | 15242 | OE2 | GLU | 2271 | 39.143 | −19.831 | 58.710 | 1.00 | 25.55 |
| ATOM | 15243 | C | GLU | 2271 | 39.241 | −18.132 | 53.704 | 1.00 | 22.11 |
| ATOM | 15244 | O | GLU | 2271 | 39.270 | −16.943 | 54.018 | 1.00 | 22.92 |
| ATOM | 15245 | N | ALA | 2272 | 40.205 | −18.703 | 52.992 | 1.00 | 23.39 |
| ATOM | 15246 | CA | ALA | 2272 | 41.370 | −17.938 | 52.558 | 1.00 | 23.06 |
| ATOM | 15247 | CB | ALA | 2272 | 42.409 | −18.878 | 51.946 | 1.00 | 23.50 |
| ATOM | 15248 | C | ALA | 2272 | 40.978 | −16.855 | 51.557 | 1.00 | 23.55 |
| ATOM | 15249 | O | ALA | 2272 | 41.648 | −15.826 | 51.455 | 1.00 | 23.19 |
| ATOM | 15250 | N | ALA | 2273 | 39.893 | −17.091 | 50.821 | 1.00 | 23.38 |
| ATOM | 15251 | CA | ALA | 2273 | 39.413 | −16.135 | 49.826 | 1.00 | 22.05 |
| ATOM | 15252 | CB | ALA | 2273 | 38.442 | −16.824 | 48.864 | 1.00 | 21.44 |
| ATOM | 15253 | C | ALA | 2273 | 38.734 | −14.935 | 50.481 | 1.00 | 21.79 |
| ATOM | 15254 | O | ALA | 2273 | 38.515 | −13.908 | 49.839 | 1.00 | 21.90 |
| ATOM | 15255 | N | GLY | 2274 | 38.390 | −15.066 | 51.758 | 1.00 | 21.57 |
| ATOM | 15256 | CA | GLY | 2274 | 37.747 | −13.961 | 52.445 | 1.00 | 21.55 |
| ATOM | 15257 | C | GLY | 2274 | 36.399 | −14.262 | 53.078 | 1.00 | 21.17 |
| ATOM | 15258 | O | GLY | 2274 | 35.818 | −13.392 | 53.725 | 1.00 | 21.65 |
| ATOM | 15259 | N | ALA | 2275 | 35.885 | −15.475 | 52.898 | 1.00 | 22.32 |
| ATOM | 15260 | CA | ALA | 2275 | 34.596 | −15.831 | 53.489 | 1.00 | 20.83 |
| ATOM | 15261 | CB | ALA | 2275 | 34.218 | −17.257 | 53.107 | 1.00 | 22.14 |
| ATOM | 15262 | C | ALA | 2275 | 34.673 | −15.698 | 55.010 | 1.00 | 21.17 |
| ATOM | 15263 | O | ALA | 2275 | 35.616 | −16.184 | 55.634 | 1.00 | 20.62 |
| ATOM | 15264 | N | GLN | 2276 | 33.684 | −15.039 | 55.604 | 1.00 | 21.24 |
| ATOM | 15265 | CA | GLN | 2276 | 33.667 | −14.850 | 57.051 | 1.00 | 21.02 |
| ATOM | 15266 | CB | GLN | 2276 | 33.293 | −13.402 | 57.378 | 1.00 | 22.86 |
| ATOM | 15267 | CG | GLN | 2276 | 34.297 | −12.396 | 56.843 | 1.00 | 24.47 |
| ATOM | 15268 | CD | GLN | 2276 | 33.899 | −10.954 | 57.096 | 1.00 | 27.97 |
| ATOM | 15269 | OE1 | GLN | 2276 | 34.022 | −10.441 | 58.212 | 1.00 | 29.78 |
| ATOM | 15270 | NE2 | GLN | 2276 | 33.418 | −10.292 | 56.055 | 1.00 | 27.80 |
| ATOM | 15271 | C | GLN | 2276 | 32.721 | −15.817 | 57.752 | 1.00 | 20.18 |
| ATOM | 15272 | O | GLN | 2276 | 32.606 | −15.819 | 58.978 | 1.00 | 18.91 |
| ATOM | 15273 | N | LEU | 2277 | 32.048 | −16.645 | 56.961 | 1.00 | 18.68 |
| ATOM | 15274 | CA | LEU | 2277 | 31.113 | −17.638 | 57.480 | 1.00 | 19.05 |
| ATOM | 15275 | CB | LEU | 2277 | 29.735 | −17.009 | 57.677 | 1.00 | 21.48 |
| ATOM | 15276 | CG | LEU | 2277 | 29.290 | −16.728 | 59.113 | 1.00 | 23.83 |
| ATOM | 15277 | CD1 | LEU | 2277 | 27.983 | −15.939 | 59.098 | 1.00 | 24.53 |
| ATOM | 15278 | CD2 | LEU | 2277 | 29.105 | −18.040 | 59.848 | 1.00 | 26.07 |
| ATOM | 15279 | C | LEU | 2277 | 30.996 | −18.787 | 56.485 | 1.00 | 18.50 |
| ATOM | 15280 | O | LEU | 2277 | 31.197 | −18.591 | 55.291 | 1.00 | 19.06 |
| ATOM | 15281 | N | LEU | 2278 | 30.669 | −19.978 | 56.978 | 1.00 | 19.37 |
| ATOM | 15282 | CA | LEU | 2278 | 30.511 | −21.141 | 56.114 | 1.00 | 20.54 |
| ATOM | 15283 | CB | LEU | 2278 | 31.795 | −21.980 | 56.080 | 1.00 | 21.74 |
| ATOM | 15284 | CG | LEU | 2278 | 31.702 | −23.321 | 55.329 | 1.00 | 23.36 |
| ATOM | 15285 | CD1 | LEU | 2278 | 31.185 | −23.110 | 53.910 | 1.00 | 21.69 |
| ATOM | 15286 | CD2 | LEU | 2278 | 33.070 | −23.977 | 55.307 | 1.00 | 23.09 |
| ATOM | 15287 | C | LEU | 2278 | 29.367 | −22.036 | 56.560 | 1.00 | 20.51 |
| ATOM | 15288 | O | LEU | 2278 | 29.232 | −22.346 | 57.746 | 1.00 | 19.68 |
| ATOM | 15289 | N | VAL | 2279 | 28.541 | −22.451 | 55.607 | 1.00 | 19.72 |
| ATOM | 15290 | CA | VAL | 2279 | 27.431 | −23.340 | 55.909 | 1.00 | 20.12 |
| ATOM | 15291 | CB | VAL | 2279 | 26.098 | −22.829 | 55.295 | 1.00 | 19.59 |
| ATOM | 15292 | CG1 | VAL | 2279 | 25.043 | −23.929 | 55.348 | 1.00 | 21.46 |
| ATOM | 15293 | CG2 | VAL | 2279 | 25.599 | −21.615 | 56.063 | 1.00 | 17.21 |
| ATOM | 15294 | C | VAL | 2279 | 27.725 | −24.731 | 55.354 | 1.00 | 19.77 |
| ATOM | 15295 | O | VAL | 2279 | 28.143 | −24.878 | 54.202 | 1.00 | 20.28 |
| ATOM | 15296 | N | LEU | 2280 | 27.527 | −25.744 | 56.191 | 1.00 | 18.40 |
| ATOM | 15297 | CA | LEU | 2280 | 27.727 | −27.133 | 55.787 | 1.00 | 19.76 |
| ATOM | 15298 | CB | LEU | 2280 | 28.710 | −27.847 | 56.717 | 1.00 | 21.85 |
| ATOM | 15299 | CG | LEU | 2280 | 30.177 | −27.418 | 56.656 | 1.00 | 24.85 |
| ATOM | 15300 | CD1 | LEU | 2280 | 30.958 | −28.145 | 57.743 | 1.00 | 25.93 |
| ATOM | 15301 | CD2 | LEU | 2280 | 30.742 | −27.745 | 55.278 | 1.00 | 25.21 |
| ATOM | 15302 | C | LEU | 2280 | 26.376 | −27.812 | 55.895 | 1.00 | 19.77 |
| ATOM | 15303 | O | LEU | 2280 | 25.774 | −27.842 | 56.968 | 1.00 | 20.23 |
| ATOM | 15304 | N | GLU | 2281 | 25.908 | −28.364 | 54.783 | 1.00 | 20.33 |
| ATOM | 15305 | CA | GLU | 2281 | 24.615 | −29.023 | 54.747 | 1.00 | 22.59 |
| ATOM | 15306 | CB | GLU | 2281 | 23.719 | −28.328 | 53.713 | 1.00 | 24.29 |
| ATOM | 15307 | CG | GLU | 2281 | 22.401 | −29.028 | 53.428 | 1.00 | 25.86 |
| ATOM | 15308 | CD | GLU | 2281 | 21.456 | −28.177 | 52.592 | 1.00 | 28.07 |
| ATOM | 15309 | OE1 | GLU | 2281 | 21.924 | −27.208 | 51.960 | 1.00 | 27.88 |
| ATOM | 15310 | OE2 | GLU | 2281 | 20.247 | −28.486 | 52.555 | 1.00 | 30.09 |
| ATOM | 15311 | C | GLU | 2281 | 24.708 | −30.513 | 54.437 | 1.00 | 23.66 |
| ATOM | 15312 | O | GLU | 2281 | 25.362 | −30.918 | 53.476 | 1.00 | 24.09 |
| ATOM | 15313 | N | CYS | 2282 | 24.050 | −31.316 | 55.271 | 1.00 | 24.83 |
| ATOM | 15314 | CA | CYS | 2282 | 24.006 | −32.764 | 55.119 | 1.00 | 26.77 |
| ATOM | 15315 | CB | CYS | 2282 | 22.988 | −33.136 | 54.048 | 1.00 | 27.38 |
| ATOM | 15316 | SG | CYS | 2282 | 21.317 | −32.574 | 54.459 | 1.00 | 27.22 |
| ATOM | 15317 | C | CYS | 2282 | 25.354 | −33.389 | 54.802 | 1.00 | 29.00 |
| ATOM | 15318 | O | CYS | 2282 | 25.623 | −33.800 | 53.670 | 1.00 | 28.72 |
| ATOM | 15319 | N | VAL | 2283 | 26.193 | −33.459 | 55.826 | 1.00 | 29.47 |
| ATOM | 15320 | CA | VAL | 2283 | 27.523 | −34.025 | 55.702 | 1.00 | 32.15 |
| ATOM | 15321 | CB | VAL | 2283 | 28.579 | −32.892 | 55.597 | 1.00 | 32.80 |
| ATOM | 15322 | CG1 | VAL | 2283 | 28.491 | −31.988 | 56.812 | 1.00 | 34.08 |
| ATOM | 15323 | CG2 | VAL | 2283 | 29.967 | −33.473 | 55.474 | 1.00 | 36.16 |
| ATOM | 15324 | C | VAL | 2283 | 27.785 | −34.866 | 56.946 | 1.00 | 31.72 |
| ATOM | 15325 | O | VAL | 2283 | 27.325 | −34.529 | 58.035 | 1.00 | 33.06 |
| ATOM | 15326 | N | PRO | 2284 | 28.510 | −35.986 | 56.799 | 1.00 | 32.37 |
| ATOM | 15327 | CD | PRO | 2284 | 29.211 | −36.504 | 55.614 | 1.00 | 31.82 |
| ATOM | 15328 | CA | PRO | 2284 | 28.787 | −36.818 | 57.970 | 1.00 | 31.61 |
| ATOM | 15329 | CB | PRO | 2284 | 29.763 | −37.866 | 57.430 | 1.00 | 31.74 |
| ATOM | 15330 | CG | PRO | 2284 | 30.393 | −37.187 | 56.241 | 1.00 | 34.69 |
| ATOM | 15331 | C | PRO | 2284 | 29.367 | −36.005 | 59.120 | 1.00 | 31.47 |
| ATOM | 15332 | O | PRO | 2284 | 30.239 | −35.155 | 58.921 | 1.00 | 30.70 |
| ATOM | 15333 | N | VAL | 2285 | 28.865 | −36.263 | 60.322 | 1.00 | 32.63 |
| ATOM | 15334 | CA | VAL | 2285 | 29.316 | −35.561 | 61.514 | 1.00 | 33.69 |
| ATOM | 15335 | CB | VAL | 2285 | 28.795 | −36.249 | 62.793 | 1.00 | 34.08 |
| ATOM | 15336 | CG1 | VAL | 2285 | 29.136 | −35.407 | 64.007 | 1.00 | 34.06 |
| ATOM | 15337 | CG2 | VAL | 2285 | 27.296 | −36.469 | 62.695 | 1.00 | 35.22 |
| ATOM | 15338 | C | VAL | 2285 | 30.835 | −35.506 | 61.583 | 1.00 | 33.95 |
| ATOM | 15339 | O | VAL | 2285 | 31.407 | −34.488 | 61.970 | 1.00 | 32.97 |
| ATOM | 15340 | N | GLU | 2286 | 31.479 | −36.606 | 61.200 | 1.00 | 35.14 |
| ATOM | 15341 | CA | GLU | 2286 | 32.939 | −36.702 | 61.225 | 1.00 | 36.30 |
| ATOM | 15342 | CB | GLU | 2286 | 33.393 | −38.045 | 60.645 | 1.00 | 38.87 |
| ATOM | 15343 | CG | GLU | 2286 | 32.532 | −39.236 | 61.031 | 1.00 | 43.96 |
| ATOM | 15344 | CD | GLU | 2286 | 32.222 | −39.277 | 62.510 | 1.00 | 45.93 |
| ATOM | 15345 | OE1 | GLU | 2286 | 33.165 | −39.136 | 63.317 | 1.00 | 49.42 |
| ATOM | 15346 | OE2 | GLU | 2286 | 31.037 | −39.458 | 62.864 | 1.00 | 46.85 |
| ATOM | 15347 | C | GLU | 2286 | 33.570 | −35.579 | 60.416 | 1.00 | 35.02 |
| ATOM | 15348 | O | GLU | 2286 | 34.452 | −34.868 | 60.898 | 1.00 | 34.61 |
| ATOM | 15349 | N | LEU | 2287 | 33.116 | −35.434 | 59.176 | 1.00 | 34.20 |
| ATOM | 15350 | CA | LEU | 2287 | 33.631 | −34.406 | 58.284 | 1.00 | 33.45 |
| ATOM | 15351 | CB | LEU | 2287 | 32.973 | −34.536 | 56.910 | 1.00 | 35.44 |
| ATOM | 15352 | CG | LEU | 2287 | 33.858 | −34.344 | 55.677 | 1.00 | 36.84 |
| ATOM | 15353 | CD1 | LEU | 2287 | 33.022 | −34.555 | 54.424 | 1.00 | 36.74 |
| ATOM | 15354 | CD2 | LEU | 2287 | 34.479 | −32.965 | 55.682 | 1.00 | 38.47 |
| ATOM | 15355 | C | LEU | 2287 | 33.363 | −33.021 | 58.869 | 1.00 | 32.89 |
| ATOM | 15356 | O | LEU | 2287 | 34.232 | −32.148 | 58.842 | 1.00 | 31.87 |
| ATOM | 15357 | N | ALA | 2288 | 32.158 | −32.826 | 59.401 | 1.00 | 32.19 |
| ATOM | 15358 | CA | ALA | 2288 | 31.779 | −31.547 | 59.999 | 1.00 | 31.97 |
| ATOM | 15359 | CB | ALA | 2288 | 30.367 | −31.630 | 60.571 | 1.00 | 31.39 |
| ATOM | 15360 | C | ALA | 2288 | 32.762 | −31.163 | 61.100 | 1.00 | 31.77 |
| ATOM | 15361 | O | ALA | 2288 | 33.092 | −29.990 | 61.272 | 1.00 | 28.78 |
| ATOM | 15362 | N | LYS | 2289 | 33.219 | −32.168 | 61.842 | 1.00 | 32.20 |
| ATOM | 15363 | CA | LYS | 2289 | 34.171 | −31.966 | 62.927 | 1.00 | 33.72 |
| ATOM | 15364 | CB | LYS | 2289 | 34.452 | −33.293 | 63.632 | 1.00 | 36.63 |
| ATOM | 15365 | CG | LYS | 2289 | 33.229 | −33.940 | 64.255 | 1.00 | 41.13 |
| ATOM | 15366 | CD | LYS | 2289 | 33.491 | −35.400 | 64.616 | 1.00 | 42.87 |
| ATOM | 15367 | CE | LYS | 2289 | 32.259 | −36.040 | 65.241 | 1.00 | 43.20 |
| ATOM | 15368 | NZ | LYS | 2289 | 32.389 | −37.516 | 65.366 | 1.00 | 42.39 |
| ATOM | 15369 | C | LYS | 2289 | 35.476 | −31.401 | 62.384 | 1.00 | 33.12 |
| ATOM | 15370 | O | LYS | 2289 | 35.966 | −30.378 | 62.863 | 1.00 | 32.46 |
| ATOM | 15371 | N | ARG | 2290 | 36.036 | −32.079 | 61.385 | 1.00 | 33.84 |
| ATOM | 15372 | CA | ARG | 2290 | 37.288 | −31.651 | 60.771 | 1.00 | 34.39 |
| ATOM | 15373 | CB | ARG | 2290 | 37.674 | −32.589 | 59.624 | 1.00 | 36.92 |
| ATOM | 15374 | CG | ARG | 2290 | 38.244 | −33.922 | 60.066 | 1.00 | 39.76 |
| ATOM | 15375 | CD | ARG | 2290 | 38.859 | −34.667 | 58.888 | 1.00 | 41.62 |
| ATOM | 15376 | NE | ARG | 2290 | 37.866 | −35.347 | 58.065 | 1.00 | 44.41 |
| ATOM | 15377 | CZ | ARG | 2290 | 38.110 | −35.840 | 56.853 | 1.00 | 45.62 |
| ATOM | 15378 | NH1 | ARG | 2290 | 39.319 | −35.722 | 56.320 | 1.00 | 45.39 |
| ATOM | 15379 | NH2 | ARG | 2290 | 37.149 | −36.460 | 56.176 | 1.00 | 46.26 |
| ATOM | 15380 | C | ARG | 2290 | 37.206 | −30.224 | 60.246 | 1.00 | 33.61 |
| ATOM | 15381 | O | ARG | 2290 | 38.051 | −29.386 | 60.567 | 1.00 | 33.99 |
| ATOM | 15382 | N | ILE | 2291 | 36.189 | −29.956 | 59.432 | 1.00 | 32.56 |
| ATOM | 15383 | CA | ILE | 2291 | 35.996 | −28.629 | 58.858 | 1.00 | 30.31 |
| ATOM | 15384 | CB | ILE | 2291 | 34.739 | −28.599 | 57.950 | 1.00 | 31.03 |
| ATOM | 15385 | CG2 | ILE | 2291 | 34.514 | −27.192 | 57.410 | 1.00 | 30.84 |
| ATOM | 15386 | CG1 | ILE | 2291 | 34.914 | −29.589 | 56.792 | 1.00 | 32.84 |
| ATOM | 15387 | CD1 | ILE | 2291 | 33.694 | −29.725 | 55.888 | 1.00 | 32.14 |
| ATOM | 15388 | C | ILE | 2291 | 35.860 | −27.568 | 59.951 | 1.00 | 29.40 |
| ATOM | 15389 | O | ILE | 2291 | 36.503 | −26.519 | 59.891 | 1.00 | 26.63 |
| ATOM | 15390 | N | THR | 2292 | 35.035 | −27.849 | 60.956 | 1.00 | 27.90 |
| ATOM | 15391 | CA | THR | 2292 | 34.819 | −26.904 | 62.051 | 1.00 | 28.75 |
| ATOM | 15392 | CB | THR | 2292 | 33.718 | −27.406 | 63.010 | 1.00 | 28.08 |
| ATOM | 15393 | OG1 | THR | 2292 | 32.489 | −27.550 | 62.286 | 1.00 | 27.32 |
| ATOM | 15394 | CG2 | THR | 2292 | 33.507 | −26.418 | 64.147 | 1.00 | 27.19 |
| ATOM | 15395 | C | THR | 2292 | 36.086 | −26.630 | 62.860 | 1.00 | 29.42 |
| ATOM | 15396 | O | THR | 2292 | 36.319 | −25.501 | 63.298 | 1.00 | 28.54 |
| ATOM | 15397 | N | GLU | 2293 | 36.900 | −27.663 | 63.052 | 1.00 | 30.75 |
| ATOM | 15398 | CA | GLU | 2293 | 38.138 | −27.529 | 63.811 | 1.00 | 32.20 |
| ATOM | 15399 | CB | GLU | 2293 | 38.578 | −28.894 | 64.348 | 1.00 | 35.28 |
| ATOM | 15400 | CG | GLU | 2293 | 37.547 | −29.570 | 65.239 | 1.00 | 39.73 |
| ATOM | 15401 | CD | GLU | 2293 | 37.976 | −30.958 | 65.686 | 1.00 | 42.81 |
| ATOM | 15402 | OE1 | GLU | 2293 | 38.390 | −31.763 | 64.822 | 1.00 | 44.94 |
| ATOM | 15403 | OE2 | GLU | 2293 | 37.889 | −31.250 | 66.899 | 1.00 | 44.47 |
| ATOM | 15404 | C | GLU | 2293 | 39.252 | −26.929 | 62.963 | 1.00 | 30.90 |
| ATOM | 15405 | O | GLU | 2293 | 40.175 | −26.306 | 63.487 | 1.00 | 31.84 |
| ATOM | 15406 | N | ALA | 2294 | 39.159 | −27.102 | 61.649 | 1.00 | 30.60 |
| ATOM | 15407 | CA | ALA | 2294 | 40.180 | −26.581 | 60.744 | 1.00 | 29.10 |
| ATOM | 15408 | CB | ALA | 2294 | 40.190 | −27.394 | 59.456 | 1.00 | 29.45 |
| ATOM | 15409 | C | ALA | 2294 | 40.015 | −25.099 | 60.417 | 1.00 | 28.58 |
| ATOM | 15410 | O | ALA | 2294 | 40.996 | −24.404 | 60.150 | 1.00 | 27.98 |
| ATOM | 15411 | N | LEU | 2295 | 38.776 | −24.620 | 60.432 | 1.00 | 26.57 |
| ATOM | 15412 | CA | LEU | 2295 | 38.501 | −23.225 | 60.111 | 1.00 | 26.11 |
| ATOM | 15413 | CB | LEU | 2295 | 37.178 | −23.108 | 59.348 | 1.00 | 26.01 |
| ATOM | 15414 | CG | LEU | 2295 | 37.097 | −23.794 | 57.987 | 1.00 | 27.63 |
| ATOM | 15415 | CD1 | LEU | 2295 | 35.761 | −23.461 | 57.323 | 1.00 | 26.37 |
| ATOM | 15416 | CD2 | LEU | 2295 | 38.242 | −23.349 | 57.117 | 1.00 | 28.50 |
| ATOM | 15417 | C | LEU | 2295 | 38.449 | −22.310 | 61.323 | 1.00 | 24.44 |
| ATOM | 15418 | O | LEU | 2295 | 37.961 | −22.692 | 62.385 | 1.00 | 25.12 |
| ATOM | 15419 | N | ALA | 2296 | 38.953 | −21.094 | 61.148 | 1.00 | 23.89 |
| ATOM | 15420 | CA | ALA | 2296 | 38.944 | −20.102 | 62.212 | 1.00 | 23.70 |
| ATOM | 15421 | CB | ALA | 2296 | 40.053 | −19.086 | 61.995 | 1.00 | 25.57 |
| ATOM | 15422 | C | ALA | 2296 | 37.591 | −19.402 | 62.200 | 1.00 | 24.70 |
| ATOM | 15423 | O | ALA | 2296 | 37.079 | −19.000 | 63.244 | 1.00 | 22.57 |
| ATOM | 15424 | N | ILE | 2297 | 37.015 | −19.253 | 61.009 | 1.00 | 23.45 |
| ATOM | 15425 | CA | ILE | 2297 | 35.715 | −18.601 | 60.877 | 1.00 | 23.32 |
| ATOM | 15426 | CB | ILE | 2297 | 35.407 | −18.223 | 59.419 | 1.00 | 22.51 |
| ATOM | 15427 | CG2 | ILE | 2297 | 36.401 | −17.190 | 58.928 | 1.00 | 23.31 |
| ATOM | 15428 | CG1 | ILE | 2297 | 35.410 | −19.481 | 58.548 | 1.00 | 22.06 |
| ATOM | 15429 | CD1 | ILE | 2297 | 34.938 | −19.252 | 57.130 | 1.00 | 23.01 |
| ATOM | 15430 | C | ILE | 2297 | 34.625 | −19.553 | 61.354 | 1.00 | 21.65 |
| ATOM | 15431 | O | ILE | 2297 | 34.782 | −20.768 | 61.288 | 1.00 | 22.46 |
| ATOM | 15432 | N | PRO | 2298 | 33.499 | −19.009 | 61.836 | 1.00 | 22.06 |
| ATOM | 15433 | CD | PRO | 2298 | 33.188 | −17.587 | 62.064 | 1.00 | 22.11 |
| ATOM | 15434 | CA | PRO | 2298 | 32.417 | −19.873 | 62.312 | 1.00 | 20.85 |
| ATOM | 15435 | CB | PRO | 2298 | 31.437 | −18.884 | 62.942 | 1.00 | 21.35 |
| ATOM | 15436 | CG | PRO | 2298 | 31.698 | −17.610 | 62.208 | 1.00 | 24.05 |
| ATOM | 15437 | C | PRO | 2298 | 31.793 | −20.728 | 61.213 | 1.00 | 22.19 |
| ATOM | 15438 | O | PRO | 2298 | 31.678 | −20.304 | 60.062 | 1.00 | 20.87 |
| ATOM | 15439 | N | VAL | 2299 | 31.416 | −21.945 | 61.585 | 1.00 | 20.48 |
| ATOM | 15440 | CA | VAL | 2299 | 30.802 | −22.899 | 60.669 | 1.00 | 21.54 |
| ATOM | 15441 | CB | VAL | 2299 | 31.628 | −24.208 | 60.616 | 1.00 | 21.85 |
| ATOM | 15442 | CG1 | VAL | 2299 | 30.961 | −25.227 | 59.704 | 1.00 | 19.09 |
| ATOM | 15443 | CG2 | VAL | 2299 | 33.035 | −23.901 | 60.144 | 1.00 | 21.05 |
| ATOM | 15444 | C | VAL | 2299 | 29.385 | −23.234 | 61.123 | 1.00 | 22.17 |
| ATOM | 15445 | O | VAL | 2299 | 29.189 | −23.842 | 62.182 | 1.00 | 23.34 |
| ATOM | 15446 | N | ILE | 2300 | 28.402 | −22.837 | 60.322 | 1.00 | 19.12 |
| ATOM | 15447 | CA | ILE | 2300 | 27.001 | −23.099 | 60.641 | 1.00 | 18.65 |
| ATOM | 15448 | CB | ILE | 2300 | 26.087 | −21.953 | 60.142 | 1.00 | 20.40 |
| ATOM | 15449 | CG2 | ILE | 2300 | 24.633 | −22.310 | 60.397 | 1.00 | 19.06 |
| ATOM | 15450 | CG1 | ILE | 2300 | 26.480 | −20.641 | 60.826 | 1.00 | 22.26 |
| ATOM | 15451 | CD1 | ILE | 2300 | 25.706 | −19.424 | 60.334 | 1.00 | 22.74 |
| ATOM | 15452 | C | ILE | 2300 | 26.566 | −24.387 | 59.966 | 1.00 | 18.15 |
| ATOM | 15453 | O | ILE | 2300 | 26.727 | −24.548 | 58.755 | 1.00 | 18.55 |
| ATOM | 15454 | N | GLY | 2301 | 26.007 | −25.305 | 60.741 | 1.00 | 16.44 |
| ATOM | 15455 | CA | GLY | 2301 | 25.581 | −26.564 | 60.168 | 1.00 | 17.23 |
| ATOM | 15456 | C | GLY | 2301 | 24.086 | −26.811 | 60.070 | 1.00 | 20.10 |
| ATOM | 15457 | O | GLY | 2301 | 23.282 | −26.228 | 60.800 | 1.00 | 17.24 |
| ATOM | 15458 | N | ILE | 2302 | 23.727 | −27.683 | 59.134 | 1.00 | 21.06 |
| ATOM | 15459 | CA | ILE | 2302 | 22.348 | −28.094 | 58.899 | 1.00 | 23.05 |
| ATOM | 15460 | CB | ILE | 2302 | 21.621 | −27.163 | 57.872 | 1.00 | 26.00 |
| ATOM | 15461 | CG2 | ILE | 2302 | 22.500 | −26.896 | 56.653 | 1.00 | 27.12 |
| ATOM | 15462 | CG1 | ILE | 2302 | 20.295 | −27.790 | 57.452 | 1.00 | 27.76 |
| ATOM | 15463 | CD1 | ILE | 2302 | 19.321 | −27.937 | 58.577 | 1.00 | 31.67 |
| ATOM | 15464 | C | ILE | 2302 | 22.458 | −29.517 | 58.358 | 1.00 | 23.20 |
| ATOM | 15465 | O | ILE | 2302 | 22.847 | −29.730 | 57.211 | 1.00 | 23.36 |
| ATOM | 15466 | N | GLY | 2303 | 22.136 | −30.493 | 59.200 | 1.00 | 24.09 |
| ATOM | 15467 | CA | GLY | 2303 | 22.254 | −31.882 | 58.791 | 1.00 | 24.67 |
| ATOM | 15468 | C | GLY | 2303 | 23.715 | −32.308 | 58.862 | 1.00 | 26.55 |
| ATOM | 15469 | O | GLY | 2303 | 24.149 | −33.218 | 58.152 | 1.00 | 25.82 |
| ATOM | 15470 | N | ALA | 2304 | 24.472 | −31.640 | 59.731 | 1.00 | 26.01 |
| ATOM | 15471 | CA | ALA | 2304 | 25.895 | −31.923 | 59.910 | 1.00 | 26.83 |
| ATOM | 15472 | CB | ALA | 2304 | 26.721 | −30.726 | 59.447 | 1.00 | 26.51 |
| ATOM | 15473 | C | ALA | 2304 | 26.247 | −32.261 | 61.357 | 1.00 | 27.49 |
| ATOM | 15474 | O | ALA | 2304 | 27.424 | −32.321 | 61.721 | 1.00 | 27.78 |
| ATOM | 15475 | N | GLY | 2305 | 25.227 | −32.470 | 62.184 | 1.00 | 29.32 |
| ATOM | 15476 | CA | GLY | 2305 | 25.465 | −32.796 | 63.580 | 1.00 | 29.51 |
| ATOM | 15477 | C | GLY | 2305 | 25.569 | −31.564 | 64.459 | 1.00 | 29.45 |
| ATOM | 15478 | O | GLY | 2305 | 25.325 | −30.447 | 63.998 | 1.00 | 29.69 |
| ATOM | 15479 | N | ASN | 2306 | 25.934 | −31.766 | 65.725 | 1.00 | 27.86 |
| ATOM | 15480 | CA | ASN | 2306 | 26.062 | −30.663 | 66.672 | 1.00 | 27.30 |
| ATOM | 15481 | CB | ASN | 2306 | 25.404 | −31.029 | 68.014 | 1.00 | 26.47 |
| ATOM | 15482 | CG | ASN | 2306 | 26.124 | −32.165 | 68.750 | 1.00 | 29.23 |
| ATOM | 15483 | OD1 | ASN | 2306 | 25.801 | −32.469 | 69.902 | 1.00 | 30.22 |
| ATOM | 15484 | ND2 | ASN | 2306 | 27.090 | −32.791 | 68.093 | 1.00 | 24.10 |
| ATOM | 15485 | C | ASN | 2306 | 27.508 | −30.240 | 66.915 | 1.00 | 27.30 |
| ATOM | 15486 | O | ASN | 2306 | 27.801 | −29.558 | 67.896 | 1.00 | 26.84 |
| ATOM | 15487 | N | VAL | 2307 | 28.406 | −30.632 | 66.014 | 1.00 | 27.99 |
| ATOM | 15488 | CA | VAL | 2307 | 29.822 | −30.302 | 66.150 | 1.00 | 27.99 |
| ATOM | 15489 | CB | VAL | 2307 | 30.706 | −31.377 | 65.491 | 1.00 | 29.98 |
| ATOM | 15490 | CG1 | VAL | 2307 | 32.168 | −31.134 | 65.843 | 1.00 | 32.46 |
| ATOM | 15491 | CG2 | VAL | 2307 | 30.272 | −32.762 | 65.947 | 1.00 | 29.99 |
| ATOM | 15492 | C | VAL | 2307 | 30.168 | −28.950 | 65.533 | 1.00 | 26.89 |
| ATOM | 15493 | O | VAL | 2307 | 31.257 | −28.414 | 65.756 | 1.00 | 25.00 |
| ATOM | 15494 | N | THR | 2308 | 29.241 | −28.402 | 64.753 | 1.00 | 25.39 |
| ATOM | 15495 | CA | THR | 2308 | 29.465 | −27.113 | 64.116 | 1.00 | 24.44 |
| ATOM | 15496 | CB | THR | 2308 | 28.486 | −26.890 | 62.930 | 1.00 | 24.99 |
| ATOM | 15497 | OG1 | THR | 2308 | 27.134 | −27.038 | 63.384 | 1.00 | 23.48 |
| ATOM | 15498 | CG2 | THR | 2308 | 28.758 | −27.897 | 61.815 | 1.00 | 23.59 |
| ATOM | 15499 | C | THR | 2308 | 29.293 | −25.990 | 65.132 | 1.00 | 23.65 |
| ATOM | 15500 | O | THR | 2308 | 28.656 | −26.169 | 66.167 | 1.00 | 25.62 |
| ATOM | 15501 | N | ASP | 2309 | 29.871 | −24.834 | 64.834 | 1.00 | 23.25 |
| ATOM | 15502 | CA | ASP | 2309 | 29.785 | −23.684 | 65.726 | 1.00 | 22.71 |
| ATOM | 15503 | CB | ASP | 2309 | 30.642 | −22.546 | 65.189 | 1.00 | 22.91 |
| ATOM | 15504 | CG | ASP | 2309 | 32.098 | −22.940 | 65.042 | 1.00 | 25.32 |
| ATOM | 15505 | OD1 | ASP | 2309 | 32.722 | −23.288 | 66.070 | 1.00 | 21.88 |
| ATOM | 15506 | OD2 | ASP | 2309 | 32.609 | −22.900 | 63.904 | 1.00 | 22.96 |
| ATOM | 15507 | C | ASP | 2309 | 28.351 | −23.206 | 65.883 | 1.00 | 22.86 |
| ATOM | 15508 | O | ASP | 2309 | 27.937 | −22.793 | 66.966 | 1.00 | 23.77 |
| ATOM | 15509 | N | GLY | 2310 | 27.592 | −23.257 | 64.798 | 1.00 | 22.07 |
| ATOM | 15510 | CA | GLY | 2310 | 26.209 | −22.814 | 64.857 | 1.00 | 21.62 |
| ATOM | 15511 | C | GLY | 2310 | 25.283 | −23.769 | 64.136 | 1.00 | 20.15 |
| ATOM | 15512 | O | GLY | 2310 | 25.731 | −24.742 | 63.533 | 1.00 | 19.40 |
| ATOM | 15513 | N | GLN | 2311 | 23.986 | −23.487 | 64.190 | 1.00 | 21.21 |
| ATOM | 15514 | CA | GLN | 2311 | 22.990 | −24.332 | 63.541 | 1.00 | 19.77 |
| ATOM | 15515 | CB | GLN | 2311 | 22.204 | −25.118 | 64.592 | 1.00 | 20.52 |
| ATOM | 15516 | CG | GLN | 2311 | 23.001 | −26.179 | 65.329 | 1.00 | 22.52 |
| ATOM | 15517 | CD | GLN | 2311 | 23.541 | −27.243 | 64.390 | 1.00 | 21.75 |
| ATOM | 15518 | OE1 | GLN | 2311 | 22.848 | −27.687 | 63.474 | 1.00 | 21.80 |
| ATOM | 15519 | NE2 | GLN | 2311 | 24.780 | −27.666 | 64.623 | 1.00 | 21.19 |
| ATOM | 15520 | C | GLN | 2311 | 22.009 | −23.485 | 62.736 | 1.00 | 20.67 |
| ATOM | 15521 | O | GLN | 2311 | 21.772 | −22.328 | 63.071 | 1.00 | 17.41 |
| ATOM | 15522 | N | ILE | 2312 | 21.447 | −24.060 | 61.676 | 1.00 | 22.09 |
| ATOM | 15523 | CA | ILE | 2312 | 20.467 | −23.343 | 60.872 | 1.00 | 24.89 |
| ATOM | 15524 | CB | ILE | 2312 | 21.110 | −22.706 | 59.606 | 1.00 | 25.72 |
| ATOM | 15525 | CG2 | ILE | 2312 | 21.564 | −23.786 | 58.635 | 1.00 | 26.02 |
| ATOM | 15526 | CG1 | ILE | 2312 | 20.100 | −21.773 | 58.932 | 1.00 | 27.58 |
| ATOM | 15527 | CD1 | ILE | 2312 | 20.741 | −20.666 | 58.103 | 1.00 | 29.83 |
| ATOM | 15528 | C | ILE | 2312 | 19.342 | −24.292 | 60.477 | 1.00 | 27.16 |
| ATOM | 15529 | O | ILE | 2312 | 19.570 | −25.481 | 60.251 | 1.00 | 28.46 |
| ATOM | 15530 | N | LEU | 2313 | 18.120 | −23.774 | 60.419 | 1.00 | 27.96 |
| ATOM | 15531 | CA | LEU | 2313 | 16.981 | −24.604 | 60.058 | 1.00 | 29.89 |
| ATOM | 15532 | CB | LEU | 2313 | 16.438 | −25.308 | 61.299 | 1.00 | 32.34 |
| ATOM | 15533 | CG | LEU | 2313 | 16.079 | −26.791 | 61.172 | 1.00 | 36.07 |
| ATOM | 15534 | CD1 | LEU | 2313 | 15.437 | −27.236 | 62.479 | 1.00 | 36.26 |
| ATOM | 15535 | CD2 | LEU | 2313 | 15.130 | −27.027 | 60.000 | 1.00 | 35.23 |
| ATOM | 15536 | C | LEU | 2313 | 15.877 | −23.765 | 59.434 | 1.00 | 29.08 |
| ATOM | 15537 | O | LEU | 2313 | 15.752 | −22.578 | 59.735 | 1.00 | 26.94 |
| ATOM | 15538 | N | VAL | 2314 | 15.088 | −24.386 | 58.558 | 1.00 | 28.13 |
| ATOM | 15539 | CA | VAL | 2314 | 13.984 | −23.699 | 57.902 | 1.00 | 26.96 |
| ATOM | 15540 | CB | VAL | 2314 | 13.493 | −24.457 | 56.647 | 1.00 | 28.27 |
| ATOM | 15541 | CG1 | VAL | 2314 | 12.350 | −23.692 | 55.999 | 1.00 | 28.30 |
| ATOM | 15542 | CG2 | VAL | 2314 | 14.638 | −24.638 | 55.666 | 1.00 | 28.58 |
| ATOM | 15543 | C | VAL | 2314 | 12.837 | −23.628 | 58.899 | 1.00 | 25.72 |
| ATOM | 15544 | O | VAL | 2314 | 12.331 | −24.654 | 59.350 | 1.00 | 25.30 |
| ATOM | 15545 | N | MET | 2315 | 12.444 | −22.411 | 59.249 | 1.00 | 25.28 |
| ATOM | 15546 | CA | MET | 2315 | 11.367 | −22.196 | 60.205 | 1.00 | 22.98 |
| ATOM | 15547 | CB | MET | 2315 | 11.025 | −20.711 | 60.278 | 1.00 | 20.69 |
| ATOM | 15548 | CG | MET | 2315 | 10.539 | −20.145 | 58.960 | 1.00 | 20.27 |
| ATOM | 15549 | SD | MET | 2315 | 9.305 | −18.854 | 59.197 | 1.00 | 19.44 |
| ATOM | 15550 | CE | MET | 2315 | 7.864 | −19.873 | 59.416 | 1.00 | 17.93 |
| ATOM | 15551 | C | MET | 2315 | 10.103 | −22.996 | 59.876 | 1.00 | 22.84 |
| ATOM | 15552 | O | MET | 2315 | 9.407 | −23.463 | 60.776 | 1.00 | 24.09 |
| ATOM | 15553 | N | HIS | 2316 | 9.807 | −23.162 | 58.591 | 1.00 | 23.24 |
| ATOM | 15554 | CA | HIS | 2316 | 8.616 | −23.903 | 58.180 | 1.00 | 22.87 |
| ATOM | 15555 | CB | HIS | 2316 | 8.445 | −23.796 | 56.662 | 1.00 | 21.29 |
| ATOM | 15556 | CG | HIS | 2316 | 7.970 | −22.449 | 56.216 | 1.00 | 20.22 |
| ATOM | 15557 | CD2 | HIS | 2316 | 8.651 | −21.318 | 55.913 | 1.00 | 17.37 |
| ATOM | 15558 | ND1 | HIS | 2316 | 6.633 | −22.120 | 56.152 | 1.00 | 20.75 |
| ATOM | 15559 | CE1 | HIS | 2316 | 6.511 | −20.844 | 55.833 | 1.00 | 20.50 |
| ATOM | 15560 | NE2 | HIS | 2316 | 7.721 | −20.333 | 55.684 | 1.00 | 20.64 |
| ATOM | 15561 | C | HIS | 2316 | 8.612 | −25.360 | 58.634 | 1.00 | 24.41 |
| ATOM | 15562 | O | HIS | 2316 | 7.551 | −25.965 | 58.787 | 1.00 | 23.54 |
| ATOM | 15563 | N | ASP | 2317 | 9.790 | −25.936 | 58.845 | 1.00 | 25.53 |
| ATOM | 15564 | CA | ASP | 2317 | 9.850 | −27.313 | 59.330 | 1.00 | 27.90 |
| ATOM | 15565 | CB | ASP | 2317 | 11.101 | −28.029 | 58.813 | 1.00 | 31.10 |
| ATOM | 15566 | CG | ASP | 2317 | 10.984 | −28.436 | 57.358 | 1.00 | 32.92 |
| ATOM | 15567 | OD1 | ASP | 2317 | 9.936 | −29.002 | 56.979 | 1.00 | 36.68 |
| ATOM | 15568 | OD2 | ASP | 2317 | 11.944 | −28.205 | 56.598 | 1.00 | 35.40 |
| ATOM | 15569 | C | ASP | 2317 | 9.874 | −27.291 | 60.857 | 1.00 | 27.46 |
| ATOM | 15570 | O | ASP | 2317 | 9.268 | −28.140 | 61.512 | 1.00 | 28.41 |
| ATOM | 15571 | N | ALA | 2318 | 10.571 | −26.304 | 61.410 | 1.00 | 28.19 |
| ATOM | 15572 | CA | ALA | 2318 | 10.703 | −26.154 | 62.853 | 1.00 | 29.17 |
| ATOM | 15573 | CB | ALA | 2318 | 11.566 | −24.946 | 63.167 | 1.00 | 31.06 |
| ATOM | 15574 | C | ALA | 2318 | 9.357 | −26.025 | 63.553 | 1.00 | 30.52 |
| ATOM | 15575 | O | ALA | 2318 | 9.230 | −26.371 | 64.727 | 1.00 | 30.59 |
| ATOM | 15576 | N | PHE | 2319 | 8.355 | −25.526 | 62.834 | 1.00 | 29.73 |
| ATOM | 15577 | CA | PHE | 2319 | 7.029 | −25.357 | 63.416 | 1.00 | 29.63 |
| ATOM | 15578 | CB | PHE | 2319 | 6.545 | −23.917 | 63.234 | 1.00 | 29.46 |
| ATOM | 15579 | CG | PHE | 2319 | 7.529 | −22.886 | 63.711 | 1.00 | 31.82 |
| ATOM | 15580 | CD1 | PHE | 2319 | 8.150 | −23.020 | 64.950 | 1.00 | 32.14 |
| ATOM | 15581 | CD2 | PHE | 2319 | 7.827 | −21.778 | 62.928 | 1.00 | 32.09 |
| ATOM | 15582 | CE1 | PHE | 2319 | 9.056 | −22.067 | 65.398 | 1.00 | 32.92 |
| ATOM | 15583 | CE2 | PHE | 2319 | 8.731 | −20.818 | 63.366 | 1.00 | 32.42 |
| ATOM | 15584 | CZ | PHE | 2319 | 9.347 | −20.963 | 64.606 | 1.00 | 33.77 |
| ATOM | 15585 | C | PHE | 2319 | 6.017 | −26.318 | 62.818 | 1.00 | 28.48 |
| ATOM | 15586 | O | PHE | 2319 | 4.811 | −26.115 | 62.941 | 1.00 | 29.39 |
| ATOM | 15587 | N | GLY | 2320 | 6.515 | −27.368 | 62.175 | 1.00 | 30.40 |
| ATOM | 15588 | CA | GLY | 2320 | 5.639 | −28.357 | 61.573 | 1.00 | 29.32 |
| ATOM | 15589 | C | GLY | 2320 | 4.618 | −27.800 | 60.597 | 1.00 | 28.92 |
| ATOM | 15590 | O | GLY | 2320 | 3.574 | −28.410 | 60.380 | 1.00 | 28.53 |
| ATOM | 15591 | N | ILE | 2321 | 4.915 | −26.647 | 60.003 | 1.00 | 28.40 |
| ATOM | 15592 | CA | ILE | 2321 | 4.001 | −26.029 | 59.047 | 1.00 | 28.56 |
| ATOM | 15593 | CB | ILE | 2321 | 4.425 | −24.573 | 58.729 | 1.00 | 26.93 |
| ATOM | 15594 | CG2 | ILE | 2321 | 3.532 | −23.991 | 57.646 | 1.00 | 28.95 |
| ATOM | 15595 | CG1 | ILE | 2321 | 4.342 | −23.721 | 60.001 | 1.00 | 27.13 |
| ATOM | 15596 | CD1 | ILE | 2321 | 4.875 | −22.326 | 59.854 | 1.00 | 23.13 |
| ATOM | 15597 | C | ILE | 2321 | 3.946 | −26.823 | 57.746 | 1.00 | 30.05 |
| ATOM | 15598 | O | ILE | 2321 | 2.876 | −27.052 | 57.190 | 1.00 | 28.53 |
| ATOM | 15599 | N | THR | 2322 | 5.106 | −27.249 | 57.264 | 1.00 | 32.75 |
| ATOM | 15600 | CA | THR | 2322 | 5.163 | −28.000 | 56.021 | 1.00 | 37.43 |
| ATOM | 15601 | CB | THR | 2322 | 6.567 | −27.946 | 55.417 | 1.00 | 38.04 |
| ATOM | 15602 | OG1 | THR | 2322 | 7.450 | −28.764 | 56.190 | 1.00 | 41.53 |
| ATOM | 15603 | CG2 | THR | 2322 | 7.084 | −26.520 | 55.431 | 1.00 | 36.66 |
| ATOM | 15604 | C | THR | 2322 | 4.775 | −29.454 | 56.242 | 1.00 | 40.21 |
| ATOM | 15605 | O | THR | 2322 | 5.297 | −30.112 | 57.143 | 1.00 | 40.84 |
| ATOM | 15606 | N | GLY | 2323 | 3.853 | −29.942 | 55.415 | 1.00 | 43.61 |
| ATOM | 15607 | CA | GLY | 2323 | 3.395 | −31.320 | 55.509 | 1.00 | 48.35 |
| ATOM | 15608 | C | GLY | 2323 | 3.586 | −31.967 | 56.868 | 1.00 | 51.33 |
| ATOM | 15609 | O | GLY | 2323 | 3.286 | −31.368 | 57.903 | 1.00 | 51.84 |
| ATOM | 15610 | N | GLY | 2324 | 4.090 | −33.196 | 56.867 | 1.00 | 53.18 |
| ATOM | 15611 | CA | GLY | 2324 | 4.309 | −33.900 | 58.116 | 1.00 | 54.96 |
| ATOM | 15612 | C | GLY | 2324 | 5.535 | −34.783 | 58.065 | 1.00 | 56.02 |
| ATOM | 15613 | O | GLY | 2324 | 5.938 | −35.358 | 59.076 | 1.00 | 57.03 |
| ATOM | 15614 | N | HIS | 2325 | 6.132 | −34.889 | 56.884 | 1.00 | 56.32 |
| ATOM | 15615 | CA | HIS | 2325 | 7.322 | −35.710 | 56.701 | 1.00 | 56.49 |
| ATOM | 15616 | CB | HIS | 2325 | 7.148 | −36.604 | 55.475 | 1.00 | 58.75 |
| ATOM | 15617 | CG | HIS | 2325 | 5.979 | −37.532 | 55.571 | 1.00 | 61.46 |
| ATOM | 15618 | CD2 | HIS | 2325 | 4.882 | −37.670 | 54.788 | 1.00 | 62.32 |
| ATOM | 15619 | ND1 | HIS | 2325 | 5.852 | −38.467 | 56.576 | 1.00 | 62.35 |
| ATOM | 15620 | CE1 | HIS | 2325 | 4.728 | −39.141 | 56.408 | 1.00 | 63.14 |
| ATOM | 15621 | NE2 | HIS | 2325 | 4.121 | −38.677 | 55.330 | 1.00 | 63.31 |
| ATOM | 15622 | C | HIS | 2325 | 8.572 | −34.852 | 56.543 | 1.00 | 55.31 |
| ATOM | 15623 | O | HIS | 2325 | 9.174 | −34.805 | 55.470 | 1.00 | 55.03 |
| ATOM | 15624 | N | ILE | 2326 | 8.959 | −34.177 | 57.622 | 1.00 | 53.27 |
| ATOM | 15625 | CA | ILE | 2326 | 10.137 | −33.318 | 57.608 | 1.00 | 50.77 |
| ATOM | 15626 | CB | ILE | 2326 | 10.230 | −32.483 | 58.898 | 1.00 | 50.89 |
| ATOM | 15627 | CG2 | ILE | 2326 | 8.991 | −31.612 | 59.043 | 1.00 | 50.89 |
| ATOM | 15628 | CG1 | ILE | 2326 | 10.380 | −33.408 | 60.107 | 1.00 | 51.06 |
| ATOM | 15629 | CD1 | ILE | 2326 | 10.620 | −32.672 | 61.410 | 1.00 | 50.63 |
| ATOM | 15630 | C | ILE | 2326 | 11.408 | −34.151 | 57.473 | 1.00 | 48.48 |
| ATOM | 15631 | O | ILE | 2326 | 11.400 | −35.356 | 57.722 | 1.00 | 48.12 |
| ATOM | 15632 | N | PRO | 2327 | 12.523 | −33.517 | 57.076 | 1.00 | 46.61 |
| ATOM | 15633 | CD | PRO | 2327 | 12.695 | −32.092 | 56.746 | 1.00 | 46.53 |
| ATOM | 15634 | CA | PRO | 2327 | 13.785 | −34.247 | 56.923 | 1.00 | 45.07 |
| ATOM | 15635 | CB | PRO | 2327 | 14.710 | −33.205 | 56.296 | 1.00 | 45.91 |
| ATOM | 15636 | CG | PRO | 2327 | 14.194 | −31.918 | 56.854 | 1.00 | 46.83 |
| ATOM | 15637 | C | PRO | 2327 | 14.317 | −34.796 | 58.245 | 1.00 | 43.14 |
| ATOM | 15638 | O | PRO | 2327 | 13.982 | −34.292 | 59.318 | 1.00 | 42.61 |
| ATOM | 15639 | N | LYS | 2328 | 15.144 | −35.833 | 58.163 | 1.00 | 41.21 |
| ATOM | 15640 | CA | LYS | 2328 | 15.716 | −36.455 | 59.354 | 1.00 | 38.78 |
| ATOM | 15641 | CB | LYS | 2328 | 16.536 | −37.692 | 58.967 | 1.00 | 41.48 |
| ATOM | 15642 | CG | LYS | 2328 | 15.696 | −38.917 | 58.617 | 1.00 | 45.03 |
| ATOM | 15643 | CD | LYS | 2328 | 14.760 | −38.644 | 57.447 | 1.00 | 47.19 |
| ATOM | 15644 | CE | LYS | 2328 | 13.770 | −39.779 | 57.245 | 1.00 | 48.48 |
| ATOM | 15645 | NZ | LYS | 2328 | 12.793 | −39.463 | 56.165 | 1.00 | 49.27 |
| ATOM | 15646 | C | LYS | 2328 | 16.583 | −35.513 | 60.181 | 1.00 | 35.61 |
| ATOM | 15647 | O | LYS | 2328 | 16.774 | −35.732 | 61.376 | 1.00 | 34.87 |
| ATOM | 15648 | N | PHE | 2329 | 17.102 | −34.463 | 59.553 | 1.00 | 31.28 |
| ATOM | 15649 | CA | PHE | 2329 | 17.955 | −33.514 | 60.262 | 1.00 | 28.57 |
| ATOM | 15650 | CB | PHE | 2329 | 19.028 | −32.967 | 59.315 | 1.00 | 28.07 |
| ATOM | 15651 | CG | PHE | 2329 | 18.480 | −32.290 | 58.089 | 1.00 | 29.05 |
| ATOM | 15652 | CD1 | PHE | 2329 | 17.954 | −31.004 | 58.157 | 1.00 | 28.21 |
| ATOM | 15653 | CD2 | PHE | 2329 | 18.506 | −32.935 | 56.857 | 1.00 | 29.00 |
| ATOM | 15654 | CE1 | PHE | 2329 | 17.470 | −30.367 | 57.011 | 1.00 | 28.14 |
| ATOM | 15655 | CE2 | PHE | 2329 | 18.026 | −32.309 | 55.708 | 1.00 | 28.09 |
| ATOM | 15656 | CZ | PHE | 2329 | 17.506 | −31.023 | 55.785 | 1.00 | 29.29 |
| ATOM | 15657 | C | PHE | 2329 | 17.179 | −32.365 | 60.896 | 1.00 | 26.22 |
| ATOM | 15658 | O | PHE | 2329 | 17.734 | −31.568 | 61.649 | 1.00 | 24.18 |
| ATOM | 15659 | N | ALA | 2330 | 15.888 | −32.288 | 60.597 | 1.00 | 26.36 |
| ATOM | 15660 | CA | ALA | 2330 | 15.051 | −31.225 | 61.133 | 1.00 | 25.12 |
| ATOM | 15661 | CB | ALA | 2330 | 14.109 | −30.721 | 60.048 | 1.00 | 24.79 |
| ATOM | 15662 | C | ALA | 2330 | 14.250 | −31.701 | 62.336 | 1.00 | 25.45 |
| ATOM | 15663 | O | ALA | 2330 | 14.066 | −32.900 | 62.540 | 1.00 | 26.65 |
| ATOM | 15664 | N | LYS | 2331 | 13.761 | −30.751 | 63.123 | 1.00 | 25.57 |
| ATOM | 15665 | CA | LYS | 2331 | 12.966 | −31.068 | 64.299 | 1.00 | 25.38 |
| ATOM | 15666 | CB | LYS | 2331 | 13.861 | −31.093 | 65.541 | 1.00 | 26.54 |
| ATOM | 15667 | CG | LYS | 2331 | 13.128 | −31.323 | 66.853 | 1.00 | 27.14 |
| ATOM | 15668 | CD | LYS | 2331 | 14.114 | −31.421 | 68.013 | 1.00 | 27.67 |
| ATOM | 15669 | CE | LYS | 2331 | 13.404 | −31.585 | 69.352 | 1.00 | 28.98 |
| ATOM | 15670 | NZ | LYS | 2331 | 14.389 | −31.782 | 70.465 | 1.00 | 25.48 |
| ATOM | 15671 | C | LYS | 2331 | 11.845 | −30.051 | 64.486 | 1.00 | 24.96 |
| ATOM | 15672 | O | LYS | 2331 | 12.060 | −28.842 | 64.371 | 1.00 | 23.50 |
| ATOM | 15673 | N | ASN | 2332 | 10.647 | −30.553 | 64.758 | 1.00 | 24.26 |
| ATOM | 15674 | CA | ASN | 2332 | 9.488 | −29.697 | 64.981 | 1.00 | 23.93 |
| ATOM | 15675 | CB | ASN | 2332 | 8.197 | −30.456 | 64.641 | 1.00 | 23.26 |
| ATOM | 15676 | CG | ASN | 2332 | 6.941 | −29.655 | 64.953 | 1.00 | 22.69 |
| ATOM | 15677 | OD1 | ASN | 2332 | 7.009 | −28.540 | 65.474 | 1.00 | 23.56 |
| ATOM | 15678 | ND2 | ASN | 2332 | 5.788 | −30.226 | 64.641 | 1.00 | 23.07 |
| ATOM | 15679 | C | ASN | 2332 | 9.469 | −29.286 | 66.446 | 1.00 | 23.54 |
| ATOM | 15680 | O | ASN | 2332 | 9.098 | −30.076 | 67.311 | 1.00 | 25.07 |
| ATOM | 15681 | N | PHE | 2333 | 9.874 | −28.054 | 66.728 | 1.00 | 25.71 |
| ATOM | 15682 | CA | PHE | 2333 | 9.894 | −27.567 | 68.103 | 1.00 | 26.02 |
| ATOM | 15683 | CB | PHE | 2333 | 10.884 | −26.411 | 68.245 | 1.00 | 27.67 |
| ATOM | 15684 | CG | PHE | 2333 | 12.317 | −26.826 | 68.102 | 1.00 | 25.56 |
| ATOM | 15685 | CD1 | PHE | 2333 | 12.885 | −26.981 | 66.846 | 1.00 | 24.93 |
| ATOM | 15686 | CD2 | PHE | 2333 | 13.091 | −27.086 | 69.224 | 1.00 | 25.58 |
| ATOM | 15687 | CE1 | PHE | 2333 | 14.210 | −27.392 | 66.706 | 1.00 | 25.54 |
| ATOM | 15688 | CE2 | PHE | 2333 | 14.416 | −27.498 | 69.098 | 1.00 | 27.73 |
| ATOM | 15689 | CZ | PHE | 2333 | 14.972 | −27.649 | 67.836 | 1.00 | 24.35 |
| ATOM | 15690 | C | PHE | 2333 | 8.529 | −27.126 | 68.606 | 1.00 | 28.07 |
| ATOM | 15691 | O | PHE | 2333 | 8.295 | −27.112 | 69.812 | 1.00 | 26.50 |
| ATOM | 15692 | N | LEU | 2334 | 7.633 | −26.759 | 67.693 | 1.00 | 28.99 |
| ATOM | 15693 | CA | LEU | 2334 | 6.296 | −26.330 | 68.095 | 1.00 | 30.49 |
| ATOM | 15694 | CB | LEU | 2334 | 5.497 | −25.820 | 66.889 | 1.00 | 28.12 |
| ATOM | 15695 | CG | LEU | 2334 | 4.023 | −25.483 | 67.170 | 1.00 | 27.45 |
| ATOM | 15696 | CD1 | LEU | 2334 | 3.933 | −24.502 | 68.323 | 1.00 | 24.55 |
| ATOM | 15697 | CD2 | LEU | 2334 | 3.363 | −24.897 | 65.924 | 1.00 | 25.02 |
| ATOM | 15698 | C | LEU | 2334 | 5.548 | −27.485 | 68.751 | 1.00 | 32.07 |
| ATOM | 15699 | O | LEU | 2334 | 4.865 | −27.297 | 69.757 | 1.00 | 32.09 |
| ATOM | 15700 | N | ALA | 2335 | 5.688 | −28.678 | 68.177 | 1.00 | 35.05 |
| ATOM | 15701 | CA | ALA | 2335 | 5.030 | −29.877 | 68.694 | 1.00 | 38.84 |
| ATOM | 15702 | CB | ALA | 2335 | 5.356 | −31.072 | 67.810 | 1.00 | 38.92 |
| ATOM | 15703 | C | ALA | 2335 | 5.452 | −30.162 | 70.130 | 1.00 | 41.82 |
| ATOM | 15704 | O | ALA | 2335 | 4.646 | −30.605 | 70.950 | 1.00 | 42.61 |
| ATOM | 15705 | N | GLU | 2336 | 6.723 | −29.907 | 70.424 | 1.00 | 45.17 |
| ATOM | 15706 | CA | GLU | 2336 | 7.273 | −30.115 | 71.761 | 1.00 | 48.42 |
| ATOM | 15707 | CB | GLU | 2336 | 8.790 | −29.899 | 71.741 | 1.00 | 50.16 |
| ATOM | 15708 | CG | GLU | 2336 | 9.571 | −30.928 | 70.936 | 1.00 | 52.50 |
| ATOM | 15709 | CD | GLU | 2336 | 9.730 | −32.246 | 71.666 | 1.00 | 53.36 |
| ATOM | 15710 | OE1 | GLU | 2336 | 8.701 | −32.871 | 72.005 | 1.00 | 54.49 |
| ATOM | 15711 | OE2 | GLU | 2336 | 10.888 | −32.655 | 71.906 | 1.00 | 54.14 |
| ATOM | 15712 | C | GLU | 2336 | 6.639 | −29.130 | 72.739 | 1.00 | 49.37 |
| ATOM | 15713 | O | GLU | 2336 | 6.895 | −29.184 | 73.941 | 1.00 | 49.41 |
| ATOM | 15714 | N | THR | 2337 | 5.816 | −28.228 | 72.211 | 1.00 | 49.87 |
| ATOM | 15715 | CA | THR | 2337 | 5.150 | −27.220 | 73.026 | 1.00 | 49.76 |
| ATOM | 15716 | CB | THR | 2337 | 6.024 | −25.946 | 73.137 | 1.00 | 50.97 |
| ATOM | 15717 | OG1 | THR | 2337 | 5.387 | −24.995 | 74.000 | 1.00 | 52.74 |
| ATOM | 15718 | CG2 | THR | 2337 | 6.235 | −25.319 | 71.764 | 1.00 | 51.29 |
| ATOM | 15719 | C | THR | 2337 | 3.793 | −26.853 | 72.424 | 1.00 | 48.80 |
| ATOM | 15720 | O | THR | 2337 | 3.150 | −27.676 | 71.768 | 1.00 | 48.94 |
| ATOM | 15721 | N | GLY | 2338 | 3.356 | −25.619 | 72.651 | 1.00 | 47.93 |
| ATOM | 15722 | CA | GLY | 2338 | 2.080 | −25.178 | 72.114 | 1.00 | 45.01 |
| ATOM | 15723 | C | GLY | 2338 | 2.136 | −23.730 | 71.680 | 1.00 | 43.22 |
| ATOM | 15724 | O | GLY | 2338 | 1.137 | −23.163 | 71.243 | 1.00 | 43.86 |
| ATOM | 15725 | N | ASP | 2339 | 3.317 | −23.134 | 71.795 | 1.00 | 40.41 |
| ATOM | 15726 | CA | ASP | 2339 | 3.518 | −21.739 | 71.426 | 1.00 | 38.45 |
| ATOM | 15727 | CB | ASP | 2339 | 3.672 | −20.895 | 72.696 | 1.00 | 40.39 |
| ATOM | 15728 | CG | ASP | 2339 | 3.966 | −19.444 | 72.402 | 1.00 | 43.35 |
| ATOM | 15729 | OD1 | ASP | 2339 | 5.137 | −19.121 | 72.115 | 1.00 | 42.97 |
| ATOM | 15730 | OD2 | ASP | 2339 | 3.018 | −18.624 | 72.448 | 1.00 | 45.56 |
| ATOM | 15731 | C | ASP | 2339 | 4.749 | −21.598 | 70.535 | 1.00 | 35.60 |
| ATOM | 15732 | O | ASP | 2339 | 5.797 | −22.194 | 70.800 | 1.00 | 33.62 |
| ATOM | 15733 | N | ILE | 2340 | 4.618 | −20.810 | 69.474 | 1.00 | 32.17 |
| ATOM | 15734 | CA | ILE | 2340 | 5.722 | −20.606 | 68.540 | 1.00 | 29.10 |
| ATOM | 15735 | CB | ILE | 2340 | 5.268 | −19.753 | 67.333 | 1.00 | 28.53 |
| ATOM | 15736 | CG2 | ILE | 2340 | 6.447 | −19.496 | 66.400 | 1.00 | 26.40 |
| ATOM | 15737 | CG1 | ILE | 2340 | 4.143 | −20.477 | 66.587 | 1.00 | 27.31 |
| ATOM | 15738 | CD1 | ILE | 2340 | 3.507 | −19.653 | 65.474 | 1.00 | 27.21 |
| ATOM | 15739 | C | ILE | 2340 | 6.930 | −19.945 | 69.202 | 1.00 | 27.86 |
| ATOM | 15740 | O | ILE | 2340 | 8.064 | −20.405 | 69.045 | 1.00 | 27.30 |
| ATOM | 15741 | N | ARG | 2341 | 6.693 | −18.867 | 69.942 | 1.00 | 25.33 |
| ATOM | 15742 | CA | ARG | 2341 | 7.788 | −18.180 | 70.611 | 1.00 | 24.52 |
| ATOM | 15743 | CB | ARG | 2341 | 7.268 | −16.927 | 71.323 | 1.00 | 23.55 |
| ATOM | 15744 | CG | ARG | 2341 | 6.902 | −15.805 | 70.349 | 1.00 | 24.38 |
| ATOM | 15745 | CD | ARG | 2341 | 6.378 | −14.561 | 71.051 | 1.00 | 27.22 |
| ATOM | 15746 | NE | ARG | 2341 | 6.060 | −13.502 | 70.091 | 1.00 | 27.15 |
| ATOM | 15747 | CZ | ARG | 2341 | 5.076 | −13.577 | 69.200 | 1.00 | 28.36 |
| ATOM | 15748 | NH1 | ARG | 2341 | 4.308 | −14.657 | 69.149 | 1.00 | 26.92 |
| ATOM | 15749 | NH2 | ARG | 2341 | 4.871 | −12.578 | 68.354 | 1.00 | 27.62 |
| ATOM | 15750 | C | ARG | 2341 | 8.499 | −19.116 | 71.584 | 1.00 | 24.05 |
| ATOM | 15751 | O | ARG | 2341 | 9.713 | −19.030 | 71.753 | 1.00 | 23.07 |
| ATOM | 15752 | N | ALA | 2342 | 7.742 | −20.019 | 72.202 | 1.00 | 23.18 |
| ATOM | 15753 | CA | ALA | 2342 | 8.307 | −20.987 | 73.138 | 1.00 | 24.94 |
| ATOM | 15754 | CB | ALA | 2342 | 7.194 | −21.728 | 73.858 | 1.00 | 24.89 |
| ATOM | 15755 | C | ALA | 2342 | 9.179 | −21.973 | 72.367 | 1.00 | 24.76 |
| ATOM | 15756 | O | ALA | 2342 | 10.204 | −22.436 | 72.865 | 1.00 | 25.19 |
| ATOM | 15757 | N | ALA | 2343 | 8.755 | −22.291 | 71.145 | 1.00 | 22.90 |
| ATOM | 15758 | CA | ALA | 2343 | 9.485 | −23.210 | 70.281 | 1.00 | 21.20 |
| ATOM | 15759 | CB | ALA | 2343 | 8.648 | −23.541 | 69.049 | 1.00 | 23.10 |
| ATOM | 15760 | C | ALA | 2343 | 10.810 | −22.574 | 69.869 | 1.00 | 20.25 |
| ATOM | 15761 | O | ALA | 2343 | 11.840 | −23.246 | 69.810 | 1.00 | 19.96 |
| ATOM | 15762 | N | VAL | 2344 | 10.774 | −21.273 | 69.586 | 1.00 | 20.13 |
| ATOM | 15763 | CA | VAL | 2344 | 11.964 | −20.530 | 69.203 | 1.00 | 20.13 |
| ATOM | 15764 | CB | VAL | 2344 | 11.612 | −19.069 | 68.829 | 1.00 | 19.76 |
| ATOM | 15765 | CG1 | VAL | 2344 | 12.883 | −18.263 | 68.605 | 1.00 | 21.02 |
| ATOM | 15766 | CG2 | VAL | 2344 | 10.751 | −19.057 | 67.560 | 1.00 | 21.33 |
| ATOM | 15767 | C | VAL | 2344 | 12.983 | −20.528 | 70.343 | 1.00 | 20.91 |
| ATOM | 15768 | O | VAL | 2344 | 14.186 | −20.685 | 70.115 | 1.00 | 18.15 |
| ATOM | 15769 | N | ARG | 2345 | 12.504 | −20.367 | 71.572 | 1.00 | 21.37 |
| ATOM | 15770 | CA | ARG | 2345 | 13.400 | −20.354 | 72.721 | 1.00 | 23.97 |
| ATOM | 15771 | CB | ARG | 2345 | 12.664 | −19.858 | 73.968 | 1.00 | 24.17 |
| ATOM | 15772 | CG | ARG | 2345 | 12.197 | −18.414 | 73.882 | 1.00 | 24.76 |
| ATOM | 15773 | CD | ARG | 2345 | 11.797 | −17.895 | 75.260 | 1.00 | 25.91 |
| ATOM | 15774 | NE | ARG | 2345 | 10.763 | −18.723 | 75.872 | 1.00 | 27.57 |
| ATOM | 15775 | CZ | ARG | 2345 | 9.465 | −18.611 | 75.617 | 1.00 | 28.92 |
| ATOM | 15776 | NH1 | ARG | 2345 | 9.027 | −17.698 | 74.758 | 1.00 | 28.97 |
| ATOM | 15777 | NH2 | ARG | 2345 | 8.604 | −19.417 | 76.218 | 1.00 | 30.22 |
| ATOM | 15778 | C | ARG | 2345 | 13.986 | −21.742 | 72.974 | 1.00 | 25.12 |
| ATOM | 15779 | O | ARG | 2345 | 15.155 | −21.870 | 73.337 | 1.00 | 26.08 |
| ATOM | 15780 | N | GLN | 2346 | 13.179 | −22.778 | 72.772 | 1.00 | 24.82 |
| ATOM | 15781 | CA | GLN | 2346 | 13.649 | −24.137 | 72.980 | 1.00 | 25.54 |
| ATOM | 15782 | CB | GLN | 2346 | 12.505 | −25.133 | 72.822 | 1.00 | 27.18 |
| ATOM | 15783 | CG | GLN | 2346 | 12.832 | −26.509 | 73.367 | 1.00 | 32.29 |
| ATOM | 15784 | CD | GLN | 2346 | 11.790 | −27.544 | 72.996 | 1.00 | 35.43 |
| ATOM | 15785 | OE1 | GLN | 2346 | 10.589 | −27.269 | 73.029 | 1.00 | 38.83 |
| ATOM | 15786 | NE2 | GLN | 2346 | 12.242 | −28.746 | 72.648 | 1.00 | 36.41 |
| ATOM | 15787 | C | GLN | 2346 | 14.753 | −24.461 | 71.974 | 1.00 | 25.43 |
| ATOM | 15788 | O | GLN | 2346 | 15.747 | −25.104 | 72.309 | 1.00 | 24.79 |
| ATOM | 15789 | N | TYR | 2347 | 14.571 | −24.008 | 70.738 | 1.00 | 24.34 |
| ATOM | 15790 | CA | TYR | 2347 | 15.555 | −24.230 | 69.685 | 1.00 | 24.09 |
| ATOM | 15791 | CB | TYR | 2347 | 15.043 | −23.659 | 68.358 | 1.00 | 24.72 |
| ATOM | 15792 | CG | TYR | 2347 | 16.087 | −23.560 | 67.259 | 1.00 | 24.42 |
| ATOM | 15793 | CD1 | TYR | 2347 | 16.883 | −24.656 | 66.919 | 1.00 | 25.35 |
| ATOM | 15794 | CE1 | TYR | 2347 | 17.814 | −24.580 | 65.878 | 1.00 | 23.82 |
| ATOM | 15795 | CD2 | TYR | 2347 | 16.248 | −22.378 | 66.533 | 1.00 | 23.75 |
| ATOM | 15796 | CE2 | TYR | 2347 | 17.174 | −22.290 | 65.489 | 1.00 | 24.65 |
| ATOM | 15797 | CZ | TYR | 2347 | 17.952 | −23.397 | 65.169 | 1.00 | 24.72 |
| ATOM | 15798 | OH | TYR | 2347 | 18.864 | −23.316 | 64.135 | 1.00 | 24.29 |
| ATOM | 15799 | C | TYR | 2347 | 16.872 | −23.569 | 70.052 | 1.00 | 24.13 |
| ATOM | 15800 | O | TYR | 2347 | 17.933 | −24.175 | 69.930 | 1.00 | 23.75 |
| ATOM | 15801 | N | MET | 2348 | 16.799 | −22.320 | 70.501 | 1.00 | 24.26 |
| ATOM | 15802 | CA | MET | 2348 | 17.996 | −21.575 | 70.887 | 1.00 | 25.61 |
| ATOM | 15803 | CB | MET | 2348 | 17.606 | −20.179 | 71.376 | 1.00 | 26.13 |
| ATOM | 15804 | CG | MET | 2348 | 17.038 | −19.273 | 70.298 | 1.00 | 26.95 |
| ATOM | 15805 | SD | MET | 2348 | 16.196 | −17.821 | 70.988 | 1.00 | 30.86 |
| ATOM | 15806 | CE | MET | 2348 | 17.540 | −17.070 | 71.910 | 1.00 | 28.64 |
| ATOM | 15807 | C | MET | 2348 | 18.748 | −22.305 | 71.990 | 1.00 | 25.38 |
| ATOM | 15808 | O | MET | 2348 | 19.973 | −22.437 | 71.944 | 1.00 | 23.27 |
| ATOM | 15809 | N | ALA | 2349 | 17.993 | −22.782 | 72.973 | 1.00 | 25.63 |
| ATOM | 15810 | CA | ALA | 2349 | 18.546 | −23.490 | 74.121 | 1.00 | 26.87 |
| ATOM | 15811 | CB | ALA | 2349 | 17.452 | −23.694 | 75.165 | 1.00 | 26.84 |
| ATOM | 15812 | C | ALA | 2349 | 19.189 | −24.832 | 73.784 | 1.00 | 27.52 |
| ATOM | 15813 | O | ALA | 2349 | 20.325 | −25.094 | 74.176 | 1.00 | 27.80 |
| ATOM | 15814 | N | GLU | 2350 | 18.461 | −25.681 | 73.065 | 1.00 | 27.55 |
| ATOM | 15815 | CA | GLU | 2350 | 18.982 | −26.998 | 72.715 | 1.00 | 28.05 |
| ATOM | 15816 | CB | GLU | 2350 | 17.908 | −27.832 | 72.020 | 1.00 | 27.67 |
| ATOM | 15817 | CG | GLU | 2350 | 16.646 | −28.041 | 72.838 | 1.00 | 30.51 |
| ATOM | 15818 | CD | GLU | 2350 | 15.822 | −29.206 | 72.325 | 1.00 | 30.48 |
| ATOM | 15819 | OE1 | GLU | 2350 | 15.869 | −29.475 | 71.108 | 1.00 | 32.21 |
| ATOM | 15820 | OE2 | GLU | 2350 | 15.124 | −29.849 | 73.135 | 1.00 | 33.22 |
| ATOM | 15821 | C | GLU | 2350 | 20.233 | −26.937 | 71.844 | 1.00 | 28.06 |
| ATOM | 15822 | O | GLU | 2350 | 21.058 | −27.849 | 71.877 | 1.00 | 26.63 |
| ATOM | 15823 | N | VAL | 2351 | 20.375 | −25.873 | 71.060 | 1.00 | 28.58 |
| ATOM | 15824 | CA | VAL | 2351 | 21.548 | −25.732 | 70.210 | 1.00 | 28.48 |
| ATOM | 15825 | CB | VAL | 2351 | 21.364 | −24.605 | 69.162 | 1.00 | 29.53 |
| ATOM | 15826 | CG1 | VAL | 2351 | 22.678 | −24.341 | 68.438 | 1.00 | 28.56 |
| ATOM | 15827 | CG2 | VAL | 2351 | 20.291 | −25.003 | 68.162 | 1.00 | 26.63 |
| ATOM | 15828 | C | VAL | 2351 | 22.751 | −25.406 | 71.091 | 1.00 | 30.61 |
| ATOM | 15829 | O | VAL | 2351 | 23.787 | −26.061 | 71.009 | 1.00 | 29.24 |
| ATOM | 15830 | N | GLU | 2352 | 22.597 | −24.404 | 71.951 | 1.00 | 31.03 |
| ATOM | 15831 | CA | GLU | 2352 | 23.676 | −23.997 | 72.840 | 1.00 | 33.56 |
| ATOM | 15832 | CB | GLU | 2352 | 23.245 | −22.796 | 73.681 | 1.00 | 34.00 |
| ATOM | 15833 | CG | GLU | 2352 | 24.353 | −22.236 | 74.554 | 1.00 | 38.30 |
| ATOM | 15834 | CD | GLU | 2352 | 23.923 | −21.011 | 75.328 | 1.00 | 40.92 |
| ATOM | 15835 | OE1 | GLU | 2352 | 24.747 | −20.485 | 76.109 | 1.00 | 42.56 |
| ATOM | 15836 | OE2 | GLU | 2352 | 22.765 | −20.571 | 75.159 | 1.00 | 42.53 |
| ATOM | 15837 | C | GLU | 2352 | 24.108 | −25.137 | 73.760 | 1.00 | 34.18 |
| ATOM | 15838 | O | GLU | 2352 | 25.286 | −25.266 | 74.090 | 1.00 | 34.72 |
| ATOM | 15839 | N | SER | 2353 | 23.149 | −25.963 | 74.166 | 1.00 | 33.74 |
| ATOM | 15840 | CA | SER | 2353 | 23.428 | −27.085 | 75.051 | 1.00 | 34.92 |
| ATOM | 15841 | CB | SER | 2353 | 22.166 | −27.483 | 75.815 | 1.00 | 35.44 |
| ATOM | 15842 | OG | SER | 2353 | 21.737 | −26.429 | 76.657 | 1.00 | 42.27 |
| ATOM | 15843 | C | SER | 2353 | 23.952 | −28.299 | 74.299 | 1.00 | 34.42 |
| ATOM | 15844 | O | SER | 2353 | 24.568 | −29.186 | 74.889 | 1.00 | 33.68 |
| ATOM | 15845 | N | GLY | 2354 | 23.703 | −28.338 | 72.995 | 1.00 | 32.46 |
| ATOM | 15846 | CA | GLY | 2354 | 24.150 | −29.463 | 72.198 | 1.00 | 31.21 |
| ATOM | 15847 | C | GLY | 2354 | 23.068 | −30.518 | 72.070 | 1.00 | 30.92 |
| ATOM | 15848 | O | GLY | 2354 | 23.251 | −31.517 | 71.373 | 1.00 | 31.34 |
| ATOM | 15849 | N | VAL | 2355 | 21.941 | −30.301 | 72.747 | 1.00 | 29.25 |
| ATOM | 15850 | CA | VAL | 2355 | 20.814 | −31.235 | 72.702 | 1.00 | 29.20 |
| ATOM | 15851 | CB | VAL | 2355 | 19.627 | −30.718 | 73.547 | 1.00 | 29.10 |
| ATOM | 15852 | CG1 | VAL | 2355 | 18.484 | −31.725 | 73.510 | 1.00 | 29.45 |
| ATOM | 15853 | CG2 | VAL | 2355 | 20.077 | −30.467 | 74.976 | 1.00 | 32.59 |
| ATOM | 15854 | C | VAL | 2355 | 20.336 | −31.413 | 71.264 | 1.00 | 27.59 |
| ATOM | 15855 | O | VAL | 2355 | 19.911 | −32.498 | 70.863 | 1.00 | 26.05 |
| ATOM | 15856 | N | TYR | 2356 | 20.399 | −30.330 | 70.498 | 1.00 | 28.08 |
| ATOM | 15857 | CA | TYR | 2356 | 19.992 | −30.352 | 69.099 | 1.00 | 26.36 |
| ATOM | 15858 | CB | TYR | 2356 | 18.713 | −29.543 | 68.889 | 1.00 | 25.86 |
| ATOM | 15859 | CG | TYR | 2356 | 18.309 | −29.492 | 67.436 | 1.00 | 25.65 |
| ATOM | 15860 | CD1 | TYR | 2356 | 17.733 | −30.602 | 66.817 | 1.00 | 25.65 |
| ATOM | 15861 | CE1 | TYR | 2356 | 17.433 | −30.596 | 65.463 | 1.00 | 25.60 |
| ATOM | 15862 | CD2 | TYR | 2356 | 18.569 | −28.363 | 66.659 | 1.00 | 24.57 |
| ATOM | 15863 | CE2 | TYR | 2356 | 18.271 | −28.345 | 65.299 | 1.00 | 24.48 |
| ATOM | 15864 | CZ | TYR | 2356 | 17.705 | −29.466 | 64.708 | 1.00 | 25.02 |
| ATOM | 15865 | OH | TYR | 2356 | 17.423 | −29.471 | 63.361 | 1.00 | 25.86 |
| ATOM | 15866 | C | TYR | 2356 | 21.099 | −29.749 | 68.245 | 1.00 | 27.03 |
| ATOM | 15867 | O | TYR | 2356 | 21.644 | −28.695 | 68.580 | 1.00 | 26.04 |
| ATOM | 15868 | N | PRO | 2357 | 21.437 | −30.405 | 67.124 | 1.00 | 29.12 |
| ATOM | 15869 | CD | PRO | 2357 | 22.345 | −29.867 | 66.094 | 1.00 | 29.21 |
| ATOM | 15870 | CA | PRO | 2357 | 20.815 | −31.650 | 66.668 | 1.00 | 30.27 |
| ATOM | 15871 | CB | PRO | 2357 | 21.151 | −31.672 | 65.182 | 1.00 | 30.86 |
| ATOM | 15872 | CG | PRO | 2357 | 22.494 | −31.034 | 65.145 | 1.00 | 30.20 |
| ATOM | 15873 | C | PRO | 2357 | 21.331 | −32.886 | 67.397 | 1.00 | 32.19 |
| ATOM | 15874 | O | PRO | 2357 | 22.491 | −32.943 | 67.804 | 1.00 | 31.74 |
| ATOM | 15875 | N | GLY | 2358 | 20.456 | −33.870 | 67.565 | 1.00 | 33.64 |
| ATOM | 15876 | CA | GLY | 2358 | 20.849 | −35.094 | 68.236 | 1.00 | 35.88 |
| ATOM | 15877 | C | GLY | 2358 | 21.322 | −36.113 | 67.222 | 1.00 | 36.96 |
| ATOM | 15878 | O | GLY | 2358 | 21.388 | −35.816 | 66.030 | 1.00 | 34.73 |
| ATOM | 15879 | N | GLU | 2359 | 21.652 | −37.313 | 67.688 | 1.00 | 38.39 |
| ATOM | 15880 | CA | GLU | 2359 | 22.114 | −38.369 | 66.795 | 1.00 | 40.49 |
| ATOM | 15881 | CB | GLU | 2359 | 22.507 | −39.612 | 67.598 | 1.00 | 42.44 |
| ATOM | 15882 | CG | GLU | 2359 | 22.814 | −40.830 | 66.733 | 1.00 | 45.21 |
| ATOM | 15883 | CD | GLU | 2359 | 23.978 | −40.605 | 65.786 | 1.00 | 46.88 |
| ATOM | 15884 | OE1 | GLU | 2359 | 24.082 | −41.352 | 64.786 | 1.00 | 47.42 |
| ATOM | 15885 | OE2 | GLU | 2359 | 24.792 | −39.691 | 66.045 | 1.00 | 48.14 |
| ATOM | 15886 | C | GLU | 2359 | 21.031 | −38.731 | 65.785 | 1.00 | 41.07 |
| ATOM | 15887 | O | GLU | 2359 | 21.328 | −39.164 | 64.669 | 1.00 | 40.53 |
| ATOM | 15888 | N | GLU | 2360 | 19.773 | −38.548 | 66.180 | 1.00 | 40.84 |
| ATOM | 15889 | CA | GLU | 2360 | 18.647 | −38.862 | 65.302 | 1.00 | 41.55 |
| ATOM | 15890 | CB | GLU | 2360 | 17.321 | −38.762 | 66.069 | 1.00 | 43.54 |
| ATOM | 15891 | CG | GLU | 2360 | 17.420 | −39.113 | 67.543 | 1.00 | 47.07 |
| ATOM | 15892 | CD | GLU | 2360 | 17.895 | −37.944 | 68.389 | 1.00 | 48.49 |
| ATOM | 15893 | OE1 | GLU | 2360 | 17.123 | −36.972 | 68.545 | 1.00 | 48.54 |
| ATOM | 15894 | OE2 | GLU | 2360 | 19.040 | −37.993 | 68.890 | 1.00 | 49.09 |
| ATOM | 15895 | C | GLU | 2360 | 18.600 | −37.912 | 64.109 | 1.00 | 40.20 |
| ATOM | 15896 | O | GLU | 2360 | 18.018 | −38.231 | 63.073 | 1.00 | 40.08 |
| ATOM | 15897 | N | HIS | 2361 | 19.224 | −36.748 | 64.261 | 1.00 | 38.76 |
| ATOM | 15898 | CA | HIS | 2361 | 19.236 | −35.735 | 63.210 | 1.00 | 37.83 |
| ATOM | 15899 | CB | HIS | 2361 | 18.893 | −34.368 | 63.809 | 1.00 | 37.62 |
| ATOM | 15900 | CG | HIS | 2361 | 17.711 | −34.387 | 64.727 | 1.00 | 37.85 |
| ATOM | 15901 | CD2 | HIS | 2361 | 17.616 | −34.163 | 66.060 | 1.00 | 37.85 |
| ATOM | 15902 | ND1 | HIS | 2361 | 16.436 | −34.685 | 64.298 | 1.00 | 37.99 |
| ATOM | 15903 | CE1 | HIS | 2361 | 15.607 | −34.644 | 65.325 | 1.00 | 37.41 |
| ATOM | 15904 | NE2 | HIS | 2361 | 16.297 | −34.329 | 66.406 | 1.00 | 37.44 |
| ATOM | 15905 | C | HIS | 2361 | 20.588 | −35.639 | 62.514 | 1.00 | 37.16 |
| ATOM | 15906 | O | HIS | 2361 | 20.815 | −34.739 | 61.704 | 1.00 | 35.47 |
| ATOM | 15907 | N | SER | 2362 | 21.479 | −36.569 | 62.829 | 1.00 | 37.15 |
| ATOM | 15908 | CA | SER | 2362 | 22.814 | −36.561 | 62.251 | 1.00 | 37.47 |
| ATOM | 15909 | CB | SER | 2362 | 23.845 | −36.519 | 63.376 | 1.00 | 37.24 |
| ATOM | 15910 | OG | SER | 2362 | 23.557 | −35.456 | 64.274 | 1.00 | 38.09 |
| ATOM | 15911 | C | SER | 2362 | 23.087 | −37.757 | 61.342 | 1.00 | 38.18 |
| ATOM | 15912 | O | SER | 2362 | 22.511 | −38.831 | 61.519 | 1.00 | 37.89 |
| ATOM | 15913 | N | PHE | 2363 | 23.966 | −37.559 | 60.363 | 1.00 | 38.60 |
| ATOM | 15914 | CA | PHE | 2363 | 24.333 | −38.617 | 59.429 | 1.00 | 39.77 |
| ATOM | 15915 | CB | PHE | 2363 | 24.257 | −38.119 | 57.983 | 1.00 | 39.50 |
| ATOM | 15916 | CG | PHE | 2363 | 22.932 | −37.524 | 57.613 | 1.00 | 39.62 |
| ATOM | 15917 | CD1 | PHE | 2363 | 22.689 | −36.168 | 57.797 | 1.00 | 40.24 |
| ATOM | 15918 | CD2 | PHE | 2363 | 21.923 | −38.318 | 57.083 | 1.00 | 39.74 |
| ATOM | 15919 | CE1 | PHE | 2363 | 21.461 | −35.608 | 57.455 | 1.00 | 41.07 |
| ATOM | 15920 | CE2 | PHE | 2363 | 20.690 | −37.769 | 56.737 | 1.00 | 40.32 |
| ATOM | 15921 | CZ | PHE | 2363 | 20.459 | −36.410 | 56.924 | 1.00 | 40.17 |
| ATOM | 15922 | C | PHE | 2363 | 25.756 | −39.089 | 59.719 | 1.00 | 40.49 |
| ATOM | 15923 | O | PHE | 2363 | 26.502 | −38.428 | 60.442 | 1.00 | 39.62 |
| ATOM | 15924 | N | HIS | 2364 | 26.129 | −40.228 | 59.145 | 1.00 | 41.96 |
| ATOM | 15925 | CA | HIS | 2364 | 27.462 | −40.791 | 59.336 | 1.00 | 44.57 |
| ATOM | 15926 | CB | HIS | 2364 | 27.468 | −41.742 | 60.532 | 1.00 | 44.77 |
| ATOM | 15927 | CG | HIS | 2364 | 27.333 | −41.053 | 61.852 | 1.00 | 44.87 |
| ATOM | 15928 | CD2 | HIS | 2364 | 26.330 | −41.057 | 62.762 | 1.00 | 45.16 |
| ATOM | 15929 | ND1 | HIS | 2364 | 28.310 | −40.227 | 62.363 | 1.00 | 45.32 |
| ATOM | 15930 | CE1 | HIS | 2364 | 27.916 | −39.752 | 63.531 | 1.00 | 45.82 |
| ATOM | 15931 | NE2 | HIS | 2364 | 26.718 | −40.241 | 63.796 | 1.00 | 45.37 |
| ATOM | 15932 | C | HIS | 2364 | 27.935 | −41.537 | 58.092 | 1.00 | 45.86 |
| ATOM | 15933 | O | HIS | 2364 | 29.117 | −41.375 | 57.723 | 1.00 | 47.15 |
| ATOM | 15934 | OXT | HIS | 2364 | 27.124 | −42.291 | 57.514 | 1.00 | 48.19 |
| ATOM | 15935 | C1 | KPL | 2365 | 18.263 | −24.454 | 54.329 | 1.00 | 39.29 |
| ATOM | 15936 | C2 | KPL | 2365 | 19.445 | −23.498 | 54.099 | 1.00 | 40.45 |
| ATOM | 15937 | C3 | KPL | 2365 | 19.514 | −22.489 | 55.256 | 1.00 | 40.25 |
| ATOM | 15938 | C4 | KPL | 2365 | 20.756 | −24.301 | 54.085 | 1.00 | 41.68 |
| ATOM | 15939 | O1 | KPL | 2365 | 20.736 | −25.261 | 53.017 | 1.00 | 44.61 |
| ATOM | 15940 | C5 | KPL | 2365 | 19.261 | −22.740 | 52.762 | 1.00 | 39.00 |
| ATOM | 15941 | O2 | KPL | 2365 | 20.101 | −22.834 | 51.890 | 1.00 | 40.74 |
| ATOM | 15942 | C6 | KPL | 2365 | 18.054 | −21.873 | 52.491 | 1.00 | 37.37 |
| ATOM | 15943 | O3 | KPL | 2365 | 17.180 | −21.735 | 53.324 | 1.00 | 37.05 |
| ATOM | 15944 | O4 | KPL | 2365 | 17.942 | −21.245 | 51.309 | 1.00 | 32.80 |
| ATOM | 15945 | CB | MET | 2401 | 40.796 | 32.161 | 43.908 | 1.00 | 71.21 |
| ATOM | 15946 | CG | MET | 2401 | 41.476 | 32.073 | 42.556 | 1.00 | 72.09 |
| ATOM | 15947 | SD | MET | 2401 | 42.294 | 33.600 | 42.104 | 1.00 | 73.69 |
| ATOM | 15948 | CE | MET | 2401 | 40.917 | 34.510 | 41.409 | 1.00 | 73.19 |
| ATOM | 15949 | C | MET | 2401 | 38.557 | 31.175 | 43.385 | 1.00 | 69.44 |
| ATOM | 15950 | O | MET | 2401 | 37.456 | 31.030 | 43.915 | 1.00 | 69.61 |
| ATOM | 15951 | N | MET | 2401 | 40.519 | 29.702 | 43.899 | 1.00 | 70.53 |
| ATOM | 15952 | CA | MET | 2401 | 39.842 | 30.999 | 44.194 | 1.00 | 70.42 |
| ATOM | 15953 | N | LYS | 2402 | 38.701 | 31.491 | 42.101 | 1.00 | 67.98 |
| ATOM | 15954 | CA | LYS | 2402 | 37.549 | 31.687 | 41.227 | 1.00 | 65.67 |
| ATOM | 15955 | CB | LYS | 2402 | 37.289 | 33.184 | 41.023 | 1.00 | 66.61 |
| ATOM | 15956 | CG | LYS | 2402 | 36.953 | 33.945 | 42.298 | 1.00 | 67.98 |
| ATOM | 15957 | CD | LYS | 2402 | 35.541 | 33.652 | 42.783 | 1.00 | 68.89 |
| ATOM | 15958 | CE | LYS | 2402 | 34.501 | 34.222 | 41.830 | 1.00 | 69.72 |
| ATOM | 15959 | NZ | LYS | 2402 | 33.116 | 34.024 | 42.339 | 1.00 | 70.87 |
| ATOM | 15960 | C | LYS | 2402 | 37.781 | 31.021 | 39.875 | 1.00 | 62.73 |
| ATOM | 15961 | O | LYS | 2402 | 38.258 | 31.656 | 38.935 | 1.00 | 63.72 |
| ATOM | 15962 | N | PRO | 2403 | 37.454 | 29.725 | 39.763 | 1.00 | 59.41 |
| ATOM | 15963 | CD | PRO | 2403 | 37.320 | 29.060 | 38.454 | 1.00 | 58.79 |
| ATOM | 15964 | CA | PRO | 2403 | 36.895 | 28.884 | 40.825 | 1.00 | 55.82 |
| ATOM | 15965 | CB | PRO | 2403 | 35.981 | 27.948 | 40.054 | 1.00 | 57.33 |
| ATOM | 15966 | CG | PRO | 2403 | 36.797 | 27.681 | 38.829 | 1.00 | 58.38 |
| ATOM | 15967 | C | PRO | 2403 | 37.987 | 28.122 | 41.576 | 1.00 | 52.24 |
| ATOM | 15968 | O | PRO | 2403 | 39.149 | 28.127 | 41.170 | 1.00 | 51.69 |
| ATOM | 15969 | N | THR | 2404 | 37.605 | 27.465 | 42.667 | 1.00 | 47.66 |
| ATOM | 15970 | CA | THR | 2404 | 38.550 | 26.690 | 43.463 | 1.00 | 43.63 |
| ATOM | 15971 | CB | THR | 2404 | 37.971 | 26.341 | 44.846 | 1.00 | 43.73 |
| ATOM | 15972 | OG1 | THR | 2404 | 37.739 | 27.544 | 45.588 | 1.00 | 43.30 |
| ATOM | 15973 | CG2 | THR | 2404 | 38.935 | 25.452 | 45.617 | 1.00 | 42.68 |
| ATOM | 15974 | C | THR | 2404 | 38.887 | 25.393 | 42.740 | 1.00 | 41.58 |
| ATOM | 15975 | O | THR | 2404 | 38.007 | 24.741 | 42.178 | 1.00 | 38.87 |
| ATOM | 15976 | N | THR | 2405 | 40.163 | 25.022 | 42.762 | 1.00 | 40.92 |
| ATOM | 15977 | CA | THR | 2405 | 40.617 | 23.807 | 42.101 | 1.00 | 40.37 |
| ATOM | 15978 | CB | THR | 2405 | 41.289 | 24.127 | 40.753 | 1.00 | 40.36 |
| ATOM | 15979 | OG1 | THR | 2405 | 42.436 | 24.956 | 40.974 | 1.00 | 40.01 |
| ATOM | 15980 | CG2 | THR | 2405 | 40.317 | 24.849 | 39.832 | 1.00 | 39.63 |
| ATOM | 15981 | C | THR | 2405 | 41.607 | 23.020 | 42.954 | 1.00 | 40.56 |
| ATOM | 15982 | O | THR | 2405 | 42.106 | 23.514 | 43.966 | 1.00 | 39.81 |
| ATOM | 15983 | N | ILE | 2406 | 41.883 | 21.790 | 42.531 | 1.00 | 40.97 |
| ATOM | 15984 | CA | ILE | 2406 | 42.815 | 20.909 | 43.228 | 1.00 | 41.27 |
| ATOM | 15985 | CB | ILE | 2406 | 43.064 | 19.621 | 42.414 | 1.00 | 41.43 |
| ATOM | 15986 | CG2 | ILE | 2406 | 43.817 | 18.602 | 43.264 | 1.00 | 39.98 |
| ATOM | 15987 | CG1 | ILE | 2406 | 41.730 | 19.036 | 41.949 | 1.00 | 42.65 |
| ATOM | 15988 | CD1 | ILE | 2406 | 41.859 | 18.074 | 40.783 | 1.00 | 44.77 |
| ATOM | 15989 | C | ILE | 2406 | 44.154 | 21.614 | 43.423 | 1.00 | 41.31 |
| ATOM | 15990 | O | ILE | 2406 | 44.771 | 21.514 | 44.482 | 1.00 | 41.10 |
| ATOM | 15991 | N | SER | 2407 | 44.595 | 22.327 | 42.392 | 1.00 | 41.82 |
| ATOM | 15992 | CA | SER | 2407 | 45.864 | 23.043 | 42.442 | 1.00 | 42.16 |
| ATOM | 15993 | CB | SER | 2407 | 46.042 | 23.881 | 41.175 | 1.00 | 42.77 |
| ATOM | 15994 | OG | SER | 2407 | 46.077 | 23.056 | 40.026 | 1.00 | 45.09 |
| ATOM | 15995 | C | SER | 2407 | 45.974 | 23.942 | 43.669 | 1.00 | 41.86 |
| ATOM | 15996 | O | SER | 2407 | 47.060 | 24.130 | 44.217 | 1.00 | 42.04 |
| ATOM | 15997 | N | LEU | 2408 | 44.846 | 24.495 | 44.101 | 1.00 | 41.20 |
| ATOM | 15998 | CA | LEU | 2408 | 44.838 | 25.374 | 45.262 | 1.00 | 40.42 |
| ATOM | 15999 | CB | LEU | 2408 | 43.483 | 26.071 | 45.390 | 1.00 | 41.71 |
| ATOM | 16000 | CG | LEU | 2408 | 43.521 | 27.558 | 45.753 | 1.00 | 42.81 |
| ATOM | 16001 | CD1 | LEU | 2408 | 42.115 | 28.034 | 46.090 | 1.00 | 42.95 |
| ATOM | 16002 | CD2 | LEU | 2408 | 44.449 | 27.786 | 46.927 | 1.00 | 43.29 |
| ATOM | 16003 | C | LEU | 2408 | 45.126 | 24.580 | 46.533 | 1.00 | 39.32 |
| ATOM | 16004 | O | LEU | 2408 | 45.921 | 25.004 | 47.372 | 1.00 | 38.69 |
| ATOM | 16005 | N | LEU | 2409 | 44.475 | 23.428 | 46.668 | 1.00 | 38.01 |
| ATOM | 16006 | CA | LEU | 2409 | 44.658 | 22.571 | 47.836 | 1.00 | 37.10 |
| ATOM | 16007 | CB | LEU | 2409 | 43.746 | 21.348 | 47.748 | 1.00 | 36.29 |
| ATOM | 16008 | CG | LEU | 2409 | 42.235 | 21.599 | 47.772 | 1.00 | 37.24 |
| ATOM | 16009 | CD1 | LEU | 2409 | 41.507 | 20.264 | 47.730 | 1.00 | 36.39 |
| ATOM | 16010 | CD2 | LEU | 2409 | 41.859 | 22.369 | 49.024 | 1.00 | 36.03 |
| ATOM | 16011 | C | LEU | 2409 | 46.103 | 22.113 | 47.956 | 1.00 | 37.48 |
| ATOM | 16012 | O | LEU | 2409 | 46.654 | 22.037 | 49.055 | 1.00 | 37.24 |
| ATOM | 16013 | N | GLN | 2410 | 46.711 | 21.805 | 46.815 | 1.00 | 37.94 |
| ATOM | 16014 | CA | GLN | 2410 | 48.096 | 21.356 | 46.779 | 1.00 | 38.43 |
| ATOM | 16015 | CB | GLN | 2410 | 48.487 | 20.989 | 45.346 | 1.00 | 38.65 |
| ATOM | 16016 | CG | GLN | 2410 | 49.854 | 20.334 | 45.206 | 1.00 | 39.36 |
| ATOM | 16017 | CD | GLN | 2410 | 49.974 | 19.041 | 45.993 | 1.00 | 40.42 |
| ATOM | 16018 | OE1 | GLN | 2410 | 50.233 | 19.054 | 47.198 | 1.00 | 40.18 |
| ATOM | 16019 | NE2 | GLN | 2410 | 49.772 | 17.912 | 45.314 | 1.00 | 39.42 |
| ATOM | 16020 | C | GLN | 2410 | 48.986 | 22.477 | 47.299 | 1.00 | 38.19 |
| ATOM | 16021 | O | GLN | 2410 | 49.927 | 22.239 | 48.057 | 1.00 | 38.73 |
| ATOM | 16022 | N | LYS | 2411 | 48.676 | 23.701 | 46.888 | 1.00 | 38.25 |
| ATOM | 16023 | CA | LYS | 2411 | 49.431 | 24.866 | 47.324 | 1.00 | 38.59 |
| ATOM | 16024 | CB | LYS | 2411 | 48.887 | 26.129 | 46.652 | 1.00 | 40.48 |
| ATOM | 16025 | CG | LYS | 2411 | 49.561 | 27.410 | 47.114 | 1.00 | 44.48 |
| ATOM | 16026 | CD | LYS | 2411 | 48.792 | 28.639 | 46.664 | 1.00 | 46.76 |
| ATOM | 16027 | CE | LYS | 2411 | 49.479 | 29.911 | 47.132 | 1.00 | 48.94 |
| ATOM | 16028 | NZ | LYS | 2411 | 48.656 | 31.123 | 46.857 | 1.00 | 51.10 |
| ATOM | 16029 | C | LYS | 2411 | 49.313 | 25.009 | 48.839 | 1.00 | 37.82 |
| ATOM | 16030 | O | LYS | 2411 | 50.300 | 25.261 | 49.533 | 1.00 | 36.98 |
| ATOM | 16031 | N | TYR | 2412 | 48.094 | 24.846 | 49.346 | 1.00 | 36.70 |
| ATOM | 16032 | CA | TYR | 2412 | 47.839 | 24.959 | 50.776 | 1.00 | 36.04 |
| ATOM | 16033 | CB | TYR | 2412 | 46.364 | 24.667 | 51.075 | 1.00 | 38.21 |
| ATOM | 16034 | CG | TYR | 2412 | 45.409 | 25.792 | 50.731 | 1.00 | 39.60 |
| ATOM | 16035 | CD1 | TYR | 2412 | 44.029 | 25.579 | 50.728 | 1.00 | 41.45 |
| ATOM | 16036 | CE1 | TYR | 2412 | 43.137 | 26.615 | 50.450 | 1.00 | 42.37 |
| ATOM | 16037 | CD2 | TYR | 2412 | 45.877 | 27.075 | 50.444 | 1.00 | 40.64 |
| ATOM | 16038 | CE2 | TYR | 2412 | 44.995 | 28.117 | 50.166 | 1.00 | 41.94 |
| ATOM | 16039 | CZ | TYR | 2412 | 43.626 | 27.879 | 50.172 | 1.00 | 42.98 |
| ATOM | 16040 | OH | TYR | 2412 | 42.746 | 28.905 | 49.908 | 1.00 | 45.55 |
| ATOM | 16041 | C | TYR | 2412 | 48.726 | 24.020 | 51.587 | 1.00 | 33.83 |
| ATOM | 16042 | O | TYR | 2412 | 49.264 | 24.408 | 52.621 | 1.00 | 32.94 |
| ATOM | 16043 | N | LYS | 2413 | 48.884 | 22.787 | 51.118 | 1.00 | 32.53 |
| ATOM | 16044 | CA | LYS | 2413 | 49.711 | 21.825 | 51.832 | 1.00 | 34.20 |
| ATOM | 16045 | CB | LYS | 2413 | 49.613 | 20.437 | 51.197 | 1.00 | 32.95 |
| ATOM | 16046 | CG | LYS | 2413 | 50.515 | 19.412 | 51.871 | 1.00 | 31.62 |
| ATOM | 16047 | CD | LYS | 2413 | 50.181 | 17.995 | 51.455 | 1.00 | 30.68 |
| ATOM | 16048 | CE | LYS | 2413 | 51.017 | 16.996 | 52.235 | 1.00 | 29.20 |
| ATOM | 16049 | NZ | LYS | 2413 | 50.512 | 15.606 | 52.122 | 1.00 | 28.10 |
| ATOM | 16050 | C | LYS | 2413 | 51.168 | 22.266 | 51.848 | 1.00 | 34.97 |
| ATOM | 16051 | O | LYS | 2413 | 51.864 | 22.101 | 52.848 | 1.00 | 33.55 |
| ATOM | 16052 | N | GLN | 2414 | 51.629 | 22.815 | 50.730 | 1.00 | 37.13 |
| ATOM | 16053 | CA | GLN | 2414 | 53.002 | 23.282 | 50.631 | 1.00 | 40.24 |
| ATOM | 16054 | CB | GLN | 2414 | 53.301 | 23.745 | 49.207 | 1.00 | 43.05 |
| ATOM | 16055 | CG | GLN | 2414 | 53.429 | 22.605 | 48.210 | 1.00 | 47.04 |
| ATOM | 16056 | CD | GLN | 2414 | 53.593 | 23.099 | 46.784 | 1.00 | 49.63 |
| ATOM | 16057 | OE1 | GLN | 2414 | 54.346 | 24.043 | 46.523 | 1.00 | 51.23 |
| ATOM | 16058 | NE2 | GLN | 2414 | 52.897 | 22.457 | 45.850 | 1.00 | 49.77 |
| ATOM | 16059 | C | GLN | 2414 | 53.228 | 24.425 | 51.609 | 1.00 | 41.12 |
| ATOM | 16060 | O | GLN | 2414 | 54.266 | 24.494 | 52.267 | 1.00 | 41.34 |
| ATOM | 16061 | N | GLU | 2415 | 52.247 | 25.315 | 51.704 | 1.00 | 41.66 |
| ATOM | 16062 | CA | GLU | 2415 | 52.325 | 26.454 | 52.609 | 1.00 | 42.25 |
| ATOM | 16063 | CB | GLU | 2415 | 51.333 | 27.536 | 52.180 | 1.00 | 43.79 |
| ATOM | 16064 | CG | GLU | 2415 | 51.495 | 27.981 | 50.740 | 1.00 | 46.53 |
| ATOM | 16065 | CD | GLU | 2415 | 50.637 | 29.182 | 50.404 | 1.00 | 47.48 |
| ATOM | 16066 | OE1 | GLU | 2415 | 49.401 | 29.100 | 50.564 | 1.00 | 48.81 |
| ATOM | 16067 | OE2 | GLU | 2415 | 51.202 | 30.212 | 49.979 | 1.00 | 49.09 |
| ATOM | 16068 | C | GLU | 2415 | 52.011 | 26.010 | 54.034 | 1.00 | 41.63 |
| ATOM | 16069 | O | GLU | 2415 | 52.000 | 26.820 | 54.964 | 1.00 | 41.75 |
| ATOM | 16070 | N | LYS | 2416 | 51.753 | 24.716 | 54.194 | 1.00 | 40.45 |
| ATOM | 16071 | CA | LYS | 2416 | 51.435 | 24.143 | 55.496 | 1.00 | 40.01 |
| ATOM | 16072 | CB | LYS | 2416 | 52.607 | 24.345 | 56.458 | 1.00 | 42.70 |
| ATOM | 16073 | CG | LYS | 2416 | 53.969 | 24.092 | 55.830 | 1.00 | 45.22 |
| ATOM | 16074 | CD | LYS | 2416 | 54.093 | 22.669 | 55.316 | 1.00 | 47.92 |
| ATOM | 16075 | CE | LYS | 2416 | 55.361 | 22.490 | 54.492 | 1.00 | 49.09 |
| ATOM | 16076 | NZ | LYS | 2416 | 56.586 | 22.872 | 55.248 | 1.00 | 50.44 |
| ATOM | 16077 | C | LYS | 2416 | 50.177 | 24.784 | 56.077 | 1.00 | 38.54 |
| ATOM | 16078 | O | LYS | 2416 | 50.012 | 24.850 | 57.295 | 1.00 | 38.60 |
| ATOM | 16079 | N | LYS | 2417 | 49.296 | 25.266 | 55.205 | 1.00 | 36.93 |
| ATOM | 16080 | CA | LYS | 2417 | 48.053 | 25.891 | 55.644 | 1.00 | 34.59 |
| ATOM | 16081 | CB | LYS | 2417 | 47.682 | 27.061 | 54.727 | 1.00 | 35.93 |
| ATOM | 16082 | CG | LYS | 2417 | 46.420 | 27.802 | 55.159 | 1.00 | 36.62 |
| ATOM | 16083 | CD | LYS | 2417 | 45.948 | 28.803 | 54.106 | 1.00 | 40.30 |
| ATOM | 16084 | CE | LYS | 2417 | 46.952 | 29.928 | 53.890 | 1.00 | 41.87 |
| ATOM | 16085 | NZ | LYS | 2417 | 46.553 | 30.824 | 52.762 | 1.00 | 42.71 |
| ATOM | 16086 | C | LYS | 2417 | 46.917 | 24.873 | 55.641 | 1.00 | 33.02 |
| ATOM | 16087 | O | LYS | 2417 | 46.510 | 24.388 | 54.584 | 1.00 | 31.46 |
| ATOM | 16088 | N | ARG | 2418 | 46.414 | 24.552 | 56.829 | 1.00 | 31.31 |
| ATOM | 16089 | CA | ARG | 2418 | 45.320 | 23.595 | 56.968 | 1.00 | 29.79 |
| ATOM | 16090 | CB | ARG | 2418 | 45.198 | 23.161 | 58.429 | 1.00 | 30.22 |
| ATOM | 16091 | CG | ARG | 2418 | 46.304 | 22.203 | 58.847 | 1.00 | 31.76 |
| ATOM | 16092 | CD | ARG | 2418 | 46.446 | 22.096 | 60.355 | 1.00 | 33.22 |
| ATOM | 16093 | NE | ARG | 2418 | 46.949 | 23.344 | 60.925 | 1.00 | 34.67 |
| ATOM | 16094 | CZ | ARG | 2418 | 47.516 | 23.446 | 62.122 | 1.00 | 34.59 |
| ATOM | 16095 | NH1 | ARG | 2418 | 47.659 | 22.371 | 62.884 | 1.00 | 35.64 |
| ATOM | 16096 | NH2 | ARG | 2418 | 47.938 | 24.623 | 62.556 | 1.00 | 33.41 |
| ATOM | 16097 | C | ARG | 2418 | 44.011 | 24.195 | 56.466 | 1.00 | 28.42 |
| ATOM | 16098 | O | ARG | 2418 | 43.638 | 25.300 | 56.854 | 1.00 | 28.67 |
| ATOM | 16099 | N | PHE | 2419 | 43.318 | 23.454 | 55.603 | 1.00 | 27.53 |
| ATOM | 16100 | CA | PHE | 2419 | 42.065 | 23.915 | 55.005 | 1.00 | 24.08 |
| ATOM | 16101 | CB | PHE | 2419 | 42.198 | 23.883 | 53.478 | 1.00 | 25.14 |
| ATOM | 16102 | CG | PHE | 2419 | 42.502 | 22.519 | 52.917 | 1.00 | 27.81 |
| ATOM | 16103 | CD1 | PHE | 2419 | 41.472 | 21.650 | 52.560 | 1.00 | 27.73 |
| ATOM | 16104 | CD2 | PHE | 2419 | 43.821 | 22.100 | 52.742 | 1.00 | 27.96 |
| ATOM | 16105 | CE1 | PHE | 2419 | 41.748 | 20.387 | 52.036 | 1.00 | 26.05 |
| ATOM | 16106 | CE2 | PHE | 2419 | 44.109 | 20.836 | 52.218 | 1.00 | 28.62 |
| ATOM | 16107 | CZ | PHE | 2419 | 43.070 | 19.978 | 51.864 | 1.00 | 27.50 |
| ATOM | 16108 | C | PHE | 2419 | 40.837 | 23.110 | 55.439 | 1.00 | 22.88 |
| ATOM | 16109 | O | PHE | 2419 | 40.930 | 21.924 | 55.734 | 1.00 | 19.16 |
| ATOM | 16110 | N | ALA | 2420 | 39.684 | 23.768 | 55.476 | 1.00 | 21.94 |
| ATOM | 16111 | CA | ALA | 2420 | 38.456 | 23.100 | 55.872 | 1.00 | 22.77 |
| ATOM | 16112 | CB | ALA | 2420 | 37.617 | 24.027 | 56.757 | 1.00 | 21.44 |
| ATOM | 16113 | C | ALA | 2420 | 37.640 | 22.656 | 54.660 | 1.00 | 22.17 |
| ATOM | 16114 | O | ALA | 2420 | 37.611 | 23.332 | 53.626 | 1.00 | 22.74 |
| ATOM | 16115 | N | THR | 2421 | 36.977 | 21.512 | 54.802 | 1.00 | 22.82 |
| ATOM | 16116 | CA | THR | 2421 | 36.138 | 20.942 | 53.750 | 1.00 | 22.02 |
| ATOM | 16117 | CB | THR | 2421 | 36.814 | 19.711 | 53.110 | 1.00 | 24.06 |
| ATOM | 16118 | OG1 | THR | 2421 | 38.061 | 20.108 | 52.514 | 1.00 | 26.62 |
| ATOM | 16119 | CG2 | THR | 2421 | 35.923 | 19.109 | 52.048 | 1.00 | 27.64 |
| ATOM | 16120 | C | THR | 2421 | 34.839 | 20.509 | 54.419 | 1.00 | 18.96 |
| ATOM | 16121 | O | THR | 2421 | 34.814 | 20.246 | 55.619 | 1.00 | 18.47 |
| ATOM | 16122 | N | ILE | 2422 | 33.759 | 20.422 | 53.656 | 1.00 | 19.02 |
| ATOM | 16123 | CA | ILE | 2422 | 32.487 | 20.040 | 54.257 | 1.00 | 17.25 |
| ATOM | 16124 | CB | ILE | 2422 | 31.764 | 21.296 | 54.811 | 1.00 | 19.41 |
| ATOM | 16125 | CG2 | ILE | 2422 | 31.183 | 22.112 | 53.667 | 1.00 | 20.48 |
| ATOM | 16126 | CG1 | ILE | 2422 | 30.664 | 20.889 | 55.792 | 1.00 | 21.17 |
| ATOM | 16127 | CD1 | ILE | 2422 | 30.129 | 22.046 | 56.604 | 1.00 | 25.73 |
| ATOM | 16128 | C | ILE | 2422 | 31.574 | 19.332 | 53.269 | 1.00 | 17.99 |
| ATOM | 16129 | O | ILE | 2422 | 31.726 | 19.474 | 52.059 | 1.00 | 16.35 |
| ATOM | 16130 | N | THR | 2423 | 30.625 | 18.569 | 53.793 | 1.00 | 17.55 |
| ATOM | 16131 | CA | THR | 2423 | 29.686 | 17.873 | 52.932 | 1.00 | 20.49 |
| ATOM | 16132 | CB | THR | 2423 | 29.163 | 16.577 | 53.580 | 1.00 | 21.53 |
| ATOM | 16133 | OG1 | THR | 2423 | 28.287 | 16.899 | 54.668 | 1.00 | 22.21 |
| ATOM | 16134 | CG2 | THR | 2423 | 30.327 | 15.738 | 54.098 | 1.00 | 26.19 |
| ATOM | 16135 | C | THR | 2423 | 28.514 | 18.815 | 52.682 | 1.00 | 19.08 |
| ATOM | 16136 | O | THR | 2423 | 28.210 | 19.678 | 53.504 | 1.00 | 18.44 |
| ATOM | 16137 | N | ALA | 2424 | 27.877 | 18.661 | 51.527 | 1.00 | 18.78 |
| ATOM | 16138 | CA | ALA | 2424 | 26.730 | 19.476 | 51.154 | 1.00 | 16.80 |
| ATOM | 16139 | CB | ALA | 2424 | 27.180 | 20.720 | 50.394 | 1.00 | 17.89 |
| ATOM | 16140 | C | ALA | 2424 | 25.843 | 18.614 | 50.269 | 1.00 | 17.29 |
| ATOM | 16141 | O | ALA | 2424 | 26.342 | 17.787 | 49.510 | 1.00 | 15.04 |
| ATOM | 16142 | N | TYR | 2425 | 24.532 | 18.795 | 50.361 | 1.00 | 16.73 |
| ATOM | 16143 | CA | TYR | 2425 | 23.633 | 17.982 | 49.547 | 1.00 | 17.37 |
| ATOM | 16144 | CB | TYR | 2425 | 23.040 | 16.852 | 50.387 | 1.00 | 16.33 |
| ATOM | 16145 | CG | TYR | 2425 | 24.006 | 16.220 | 51.351 | 1.00 | 17.54 |
| ATOM | 16146 | CD1 | TYR | 2425 | 23.974 | 16.546 | 52.704 | 1.00 | 18.85 |
| ATOM | 16147 | CE1 | TYR | 2425 | 24.846 | 15.954 | 53.605 | 1.00 | 19.50 |
| ATOM | 16148 | CD2 | TYR | 2425 | 24.946 | 15.285 | 50.917 | 1.00 | 17.67 |
| ATOM | 16149 | CE2 | TYR | 2425 | 25.829 | 14.684 | 51.815 | 1.00 | 18.16 |
| ATOM | 16150 | CZ | TYR | 2425 | 25.766 | 15.026 | 53.160 | 1.00 | 19.76 |
| ATOM | 16151 | OH | TYR | 2425 | 26.608 | 14.418 | 54.065 | 1.00 | 21.42 |
| ATOM | 16152 | C | TYR | 2425 | 22.496 | 18.760 | 48.908 | 1.00 | 18.27 |
| ATOM | 16153 | O | TYR | 2425 | 21.639 | 18.172 | 48.248 | 1.00 | 20.53 |
| ATOM | 16154 | N | ASP | 2426 | 22.477 | 20.074 | 49.101 | 1.00 | 17.43 |
| ATOM | 16155 | CA | ASP | 2426 | 21.420 | 20.899 | 48.525 | 1.00 | 17.57 |
| ATOM | 16156 | CB | ASP | 2426 | 20.228 | 20.964 | 49.486 | 1.00 | 17.38 |
| ATOM | 16157 | CG | ASP | 2426 | 20.581 | 21.617 | 50.824 | 1.00 | 18.75 |
| ATOM | 16158 | OD1 | ASP | 2426 | 20.752 | 22.849 | 50.861 | 1.00 | 20.93 |
| ATOM | 16159 | OD2 | ASP | 2426 | 20.687 | 20.892 | 51.836 | 1.00 | 19.12 |
| ATOM | 16160 | C | ASP | 2426 | 21.904 | 22.309 | 48.195 | 1.00 | 19.13 |
| ATOM | 16161 | O | ASP | 2426 | 22.985 | 22.720 | 48.615 | 1.00 | 19.45 |
| ATOM | 16162 | N | TYR | 2427 | 21.084 | 23.040 | 47.448 | 1.00 | 17.80 |
| ATOM | 16163 | CA | TYR | 2427 | 21.385 | 24.404 | 47.026 | 1.00 | 20.14 |
| ATOM | 16164 | CB | TYR | 2427 | 20.232 | 24.941 | 46.175 | 1.00 | 19.94 |
| ATOM | 16165 | CG | TYR | 2427 | 20.321 | 26.420 | 45.862 | 1.00 | 20.70 |
| ATOM | 16166 | CD1 | TYR | 2427 | 21.142 | 26.891 | 44.838 | 1.00 | 20.50 |
| ATOM | 16167 | CE1 | TYR | 2427 | 21.222 | 28.253 | 44.547 | 1.00 | 23.68 |
| ATOM | 16168 | CD2 | TYR | 2427 | 19.581 | 27.351 | 46.594 | 1.00 | 23.01 |
| ATOM | 16169 | CE2 | TYR | 2427 | 19.652 | 28.711 | 46.314 | 1.00 | 23.40 |
| ATOM | 16170 | CZ | TYR | 2427 | 20.470 | 29.156 | 45.291 | 1.00 | 24.74 |
| ATOM | 16171 | OH | TYR | 2427 | 20.526 | 30.499 | 45.006 | 1.00 | 28.18 |
| ATOM | 16172 | C | TYR | 2427 | 21.630 | 25.382 | 48.174 | 1.00 | 20.13 |
| ATOM | 16173 | O | TYR | 2427 | 22.609 | 26.124 | 48.163 | 1.00 | 19.70 |
| ATOM | 16174 | N | SER | 2428 | 20.727 | 25.379 | 49.148 | 1.00 | 20.69 |
| ATOM | 16175 | CA | SER | 2428 | 20.794 | 26.288 | 50.287 | 1.00 | 20.45 |
| ATOM | 16176 | CB | SER | 2428 | 19.592 | 26.055 | 51.200 | 1.00 | 21.08 |
| ATOM | 16177 | OG | SER | 2428 | 18.394 | 26.357 | 50.511 | 1.00 | 19.23 |
| ATOM | 16178 | C | SER | 2428 | 22.077 | 26.252 | 51.107 | 1.00 | 20.33 |
| ATOM | 16179 | O | SER | 2428 | 22.717 | 27.289 | 51.328 | 1.00 | 19.96 |
| ATOM | 16180 | N | PHE | 2429 | 22.456 | 25.073 | 51.580 | 1.00 | 18.92 |
| ATOM | 16181 | CA | PHE | 2429 | 23.678 | 24.976 | 52.363 | 1.00 | 18.46 |
| ATOM | 16182 | CB | PHE | 2429 | 23.739 | 23.643 | 53.120 | 1.00 | 17.47 |
| ATOM | 16183 | CG | PHE | 2429 | 22.916 | 23.633 | 54.381 | 1.00 | 18.24 |
| ATOM | 16184 | CD1 | PHE | 2429 | 21.682 | 22.986 | 54.427 | 1.00 | 17.48 |
| ATOM | 16185 | CD2 | PHE | 2429 | 23.370 | 24.296 | 55.524 | 1.00 | 15.99 |
| ATOM | 16186 | CE1 | PHE | 2429 | 20.911 | 22.995 | 55.590 | 1.00 | 17.58 |
| ATOM | 16187 | CE2 | PHE | 2429 | 22.611 | 24.312 | 56.686 | 1.00 | 13.97 |
| ATOM | 16188 | CZ | PHE | 2429 | 21.376 | 23.661 | 56.727 | 1.00 | 17.59 |
| ATOM | 16189 | C | PHE | 2429 | 24.917 | 25.174 | 51.496 | 1.00 | 19.44 |
| ATOM | 16190 | O | PHE | 2429 | 25.885 | 25.813 | 51.924 | 1.00 | 18.28 |
| ATOM | 16191 | N | ALA | 2430 | 24.892 | 24.653 | 50.271 | 1.00 | 20.34 |
| ATOM | 16192 | CA | ALA | 2430 | 26.035 | 24.831 | 49.381 | 1.00 | 22.75 |
| ATOM | 16193 | CB | ALA | 2430 | 25.805 | 24.092 | 48.064 | 1.00 | 22.82 |
| ATOM | 16194 | C | ALA | 2430 | 26.251 | 26.325 | 49.119 | 1.00 | 23.92 |
| ATOM | 16195 | O | ALA | 2430 | 27.388 | 26.792 | 49.003 | 1.00 | 25.09 |
| ATOM | 16196 | N | LYS | 2431 | 25.148 | 27.065 | 49.037 | 1.00 | 24.33 |
| ATOM | 16197 | CA | LYS | 2431 | 25.180 | 28.506 | 48.797 | 1.00 | 25.15 |
| ATOM | 16198 | CB | LYS | 2431 | 23.767 | 28.999 | 48.460 | 1.00 | 27.16 |
| ATOM | 16199 | CG | LYS | 2431 | 23.646 | 30.478 | 48.124 | 1.00 | 29.73 |
| ATOM | 16200 | CD | LYS | 2431 | 24.318 | 30.816 | 46.811 | 1.00 | 34.33 |
| ATOM | 16201 | CE | LYS | 2431 | 23.775 | 32.117 | 46.241 | 1.00 | 37.47 |
| ATOM | 16202 | NZ | LYS | 2431 | 23.956 | 33.264 | 47.174 | 1.00 | 39.95 |
| ATOM | 16203 | C | LYS | 2431 | 25.712 | 29.246 | 50.027 | 1.00 | 24.27 |
| ATOM | 16204 | O | LYS | 2431 | 26.546 | 30.145 | 49.918 | 1.00 | 23.43 |
| ATOM | 16205 | N | LEU | 2432 | 25.219 | 28.858 | 51.198 | 1.00 | 23.70 |
| ATOM | 16206 | CA | LEU | 2432 | 25.641 | 29.468 | 52.451 | 1.00 | 23.86 |
| ATOM | 16207 | CB | LEU | 2432 | 24.812 | 28.898 | 53.609 | 1.00 | 21.16 |
| ATOM | 16208 | CG | LEU | 2432 | 25.055 | 29.415 | 55.035 | 1.00 | 19.80 |
| ATOM | 16209 | CD1 | LEU | 2432 | 23.849 | 29.079 | 55.909 | 1.00 | 16.46 |
| ATOM | 16210 | CD2 | LEU | 2432 | 26.322 | 28.796 | 55.613 | 1.00 | 16.28 |
| ATOM | 16211 | C | LEU | 2432 | 27.132 | 29.240 | 52.697 | 1.00 | 24.74 |
| ATOM | 16212 | O | LEU | 2432 | 27.825 | 30.134 | 53.180 | 1.00 | 25.72 |
| ATOM | 16213 | N | PHE | 2433 | 27.630 | 28.051 | 52.367 | 1.00 | 25.69 |
| ATOM | 16214 | CA | PHE | 2433 | 29.049 | 27.752 | 52.567 | 1.00 | 25.88 |
| ATOM | 16215 | CB | PHE | 2433 | 29.343 | 26.259 | 52.358 | 1.00 | 25.66 |
| ATOM | 16216 | CG | PHE | 2433 | 28.615 | 25.342 | 53.307 | 1.00 | 23.12 |
| ATOM | 16217 | CD1 | PHE | 2433 | 28.351 | 25.728 | 54.621 | 1.00 | 20.93 |
| ATOM | 16218 | CD2 | PHE | 2433 | 28.204 | 24.076 | 52.884 | 1.00 | 22.54 |
| ATOM | 16219 | CE1 | PHE | 2433 | 27.688 | 24.873 | 55.499 | 1.00 | 21.36 |
| ATOM | 16220 | CE2 | PHE | 2433 | 27.542 | 23.214 | 53.756 | 1.00 | 21.24 |
| ATOM | 16221 | CZ | PHE | 2433 | 27.281 | 23.613 | 55.068 | 1.00 | 23.36 |
| ATOM | 16222 | C | PHE | 2433 | 29.918 | 28.557 | 51.606 | 1.00 | 28.10 |
| ATOM | 16223 | O | PHE | 2433 | 30.890 | 29.192 | 52.019 | 1.00 | 26.80 |
| ATOM | 16224 | N | ALA | 2434 | 29.575 | 28.520 | 50.319 | 1.00 | 28.19 |
| ATOM | 16225 | CA | ALA | 2434 | 30.341 | 29.250 | 49.314 | 1.00 | 29.37 |
| ATOM | 16226 | CB | ALA | 2434 | 29.714 | 29.072 | 47.934 | 1.00 | 28.66 |
| ATOM | 16227 | C | ALA | 2434 | 30.424 | 30.731 | 49.665 | 1.00 | 29.43 |
| ATOM | 16228 | O | ALA | 2434 | 31.465 | 31.358 | 49.480 | 1.00 | 29.86 |
| ATOM | 16229 | N | ASP | 2435 | 29.331 | 31.289 | 50.176 | 1.00 | 29.97 |
| ATOM | 16230 | CA | ASP | 2435 | 29.320 | 32.702 | 50.540 | 1.00 | 32.22 |
| ATOM | 16231 | CB | ASP | 2435 | 27.893 | 33.205 | 50.766 | 1.00 | 33.58 |
| ATOM | 16232 | CG | ASP | 2435 | 27.021 | 33.062 | 49.539 | 1.00 | 36.19 |
| ATOM | 16233 | OD1 | ASP | 2435 | 27.540 | 33.202 | 48.409 | 1.00 | 37.94 |
| ATOM | 16234 | OD2 | ASP | 2435 | 25.807 | 32.825 | 49.706 | 1.00 | 38.98 |
| ATOM | 16235 | C | ASP | 2435 | 30.143 | 32.997 | 51.787 | 1.00 | 32.06 |
| ATOM | 16236 | O | ASP | 2435 | 30.372 | 34.161 | 52.117 | 1.00 | 32.90 |
| ATOM | 16237 | N | GLU | 2436 | 30.580 | 31.955 | 52.486 | 1.00 | 31.24 |
| ATOM | 16238 | CA | GLU | 2436 | 31.376 | 32.149 | 53.687 | 1.00 | 31.97 |
| ATOM | 16239 | CB | GLU | 2436 | 30.816 | 31.326 | 54.850 | 1.00 | 32.11 |
| ATOM | 16240 | CG | GLU | 2436 | 29.464 | 31.801 | 55.357 | 1.00 | 33.39 |
| ATOM | 16241 | CD | GLU | 2436 | 29.453 | 33.285 | 55.680 | 1.00 | 33.83 |
| ATOM | 16242 | OE1 | GLU | 2436 | 30.357 | 33.745 | 56.411 | 1.00 | 35.70 |
| ATOM | 16243 | OE2 | GLU | 2436 | 28.537 | 33.987 | 55.208 | 1.00 | 33.76 |
| ATOM | 16244 | C | GLU | 2436 | 32.836 | 31.790 | 53.472 | 1.00 | 32.46 |
| ATOM | 16245 | O | GLU | 2436 | 33.659 | 31.983 | 54.362 | 1.00 | 32.04 |
| ATOM | 16246 | N | GLY | 2437 | 33.162 | 31.265 | 52.294 | 1.00 | 32.25 |
| ATOM | 16247 | CA | GLY | 2437 | 34.542 | 30.904 | 52.026 | 1.00 | 33.78 |
| ATOM | 16248 | C | GLY | 2437 | 34.786 | 29.425 | 51.797 | 1.00 | 33.68 |
| ATOM | 16249 | O | GLY | 2437 | 35.759 | 29.062 | 51.143 | 1.00 | 36.34 |
| ATOM | 16250 | N | LEU | 2438 | 33.927 | 28.569 | 52.347 | 1.00 | 32.66 |
| ATOM | 16251 | CA | LEU | 2438 | 34.066 | 27.127 | 52.161 | 1.00 | 32.08 |
| ATOM | 16252 | CB | LEU | 2438 | 33.059 | 26.372 | 53.031 | 1.00 | 32.00 |
| ATOM | 16253 | CG | LEU | 2438 | 33.606 | 25.758 | 54.314 | 1.00 | 31.91 |
| ATOM | 16254 | CD1 | LEU | 2438 | 32.472 | 25.144 | 55.116 | 1.00 | 31.31 |
| ATOM | 16255 | CD2 | LEU | 2438 | 34.656 | 24.708 | 53.977 | 1.00 | 32.09 |
| ATOM | 16256 | C | LEU | 2438 | 33.810 | 26.810 | 50.697 | 1.00 | 30.44 |
| ATOM | 16257 | O | LEU | 2438 | 32.659 | 26.721 | 50.267 | 1.00 | 30.98 |
| ATOM | 16258 | N | ASN | 2439 | 34.889 | 26.641 | 49.940 | 1.00 | 29.40 |
| ATOM | 16259 | CA | ASN | 2439 | 34.788 | 26.370 | 48.513 | 1.00 | 28.49 |
| ATOM | 16260 | CB | ASN | 2439 | 35.721 | 27.306 | 47.738 | 1.00 | 31.32 |
| ATOM | 16261 | CG | ASN | 2439 | 35.549 | 28.764 | 48.134 | 1.00 | 34.03 |
| ATOM | 16262 | OD1 | ASN | 2439 | 34.435 | 29.292 | 48.140 | 1.00 | 36.11 |
| ATOM | 16263 | ND2 | ASN | 2439 | 36.655 | 29.422 | 48.461 | 1.00 | 35.10 |
| ATOM | 16264 | C | ASN | 2439 | 35.122 | 24.922 | 48.173 | 1.00 | 26.24 |
| ATOM | 16265 | O | ASN | 2439 | 35.344 | 24.593 | 47.012 | 1.00 | 25.25 |
| ATOM | 16266 | N | VAL | 2440 | 35.155 | 24.065 | 49.188 | 1.00 | 24.29 |
| ATOM | 16267 | CA | VAL | 2440 | 35.446 | 22.657 | 48.985 | 1.00 | 24.09 |
| ATOM | 16268 | CB | VAL | 2440 | 36.774 | 22.248 | 49.640 | 1.00 | 25.05 |
| ATOM | 16269 | CG1 | VAL | 2440 | 37.067 | 20.788 | 49.345 | 1.00 | 24.91 |
| ATOM | 16270 | CG2 | VAL | 2440 | 37.896 | 23.126 | 49.114 | 1.00 | 25.37 |
| ATOM | 16271 | C | VAL | 2440 | 34.323 | 21.843 | 49.597 | 1.00 | 22.62 |
| ATOM | 16272 | O | VAL | 2440 | 34.228 | 21.711 | 50.816 | 1.00 | 19.04 |
| ATOM | 16273 | N | MET | 2441 | 33.471 | 21.297 | 48.737 | 1.00 | 21.81 |
| ATOM | 16274 | CA | MET | 2441 | 32.322 | 20.528 | 49.183 | 1.00 | 20.07 |
| ATOM | 16275 | CB | MET | 2441 | 31.033 | 21.250 | 48.785 | 1.00 | 22.11 |
| ATOM | 16276 | CG | MET | 2441 | 30.682 | 22.414 | 49.695 | 1.00 | 23.98 |
| ATOM | 16277 | SD | MET | 2441 | 29.512 | 23.563 | 48.974 | 1.00 | 25.48 |
| ATOM | 16278 | CE | MET | 2441 | 30.566 | 25.021 | 48.739 | 1.00 | 24.85 |
| ATOM | 16279 | C | MET | 2441 | 32.295 | 19.118 | 48.637 | 1.00 | 20.47 |
| ATOM | 16280 | O | MET | 2441 | 32.712 | 18.867 | 47.508 | 1.00 | 19.61 |
| ATOM | 16281 | N | LEU | 2442 | 31.786 | 18.200 | 49.450 | 1.00 | 19.57 |
| ATOM | 16282 | CA | LEU | 2442 | 31.689 | 16.805 | 49.055 | 1.00 | 20.14 |
| ATOM | 16283 | CB | LEU | 2442 | 32.576 | 15.951 | 49.964 | 1.00 | 20.82 |
| ATOM | 16284 | CG | LEU | 2442 | 32.654 | 14.421 | 49.855 | 1.00 | 24.63 |
| ATOM | 16285 | CD1 | LEU | 2442 | 31.547 | 13.810 | 50.661 | 1.00 | 25.41 |
| ATOM | 16286 | CD2 | LEU | 2442 | 32.611 | 13.956 | 48.399 | 1.00 | 22.10 |
| ATOM | 16287 | C | LEU | 2442 | 30.254 | 16.307 | 49.104 | 1.00 | 18.06 |
| ATOM | 16288 | O | LEU | 2442 | 29.556 | 16.453 | 50.109 | 1.00 | 15.33 |
| ATOM | 16289 | N | VAL | 2443 | 29.816 | 15.730 | 47.998 | 1.00 | 16.74 |
| ATOM | 16290 | CA | VAL | 2443 | 28.481 | 15.155 | 47.931 | 1.00 | 18.13 |
| ATOM | 16291 | CB | VAL | 2443 | 27.850 | 15.367 | 46.553 | 1.00 | 18.92 |
| ATOM | 16292 | CG1 | VAL | 2443 | 26.449 | 14.756 | 46.527 | 1.00 | 18.84 |
| ATOM | 16293 | CG2 | VAL | 2443 | 27.797 | 16.855 | 46.235 | 1.00 | 20.62 |
| ATOM | 16294 | C | VAL | 2443 | 28.743 | 13.675 | 48.162 | 1.00 | 18.17 |
| ATOM | 16295 | O | VAL | 2443 | 28.939 | 12.918 | 47.214 | 1.00 | 18.72 |
| ATOM | 16296 | N | GLY | 2444 | 28.759 | 13.278 | 49.432 | 1.00 | 17.59 |
| ATOM | 16297 | CA | GLY | 2444 | 29.047 | 11.901 | 49.773 | 1.00 | 17.73 |
| ATOM | 16298 | C | GLY | 2444 | 27.848 | 11.015 | 50.003 | 1.00 | 19.75 |
| ATOM | 16299 | O | GLY | 2444 | 26.722 | 11.499 | 50.162 | 1.00 | 18.97 |
| ATOM | 16300 | N | ASP | 2445 | 28.083 | 9.706 | 50.019 | 1.00 | 19.18 |
| ATOM | 16301 | CA | ASP | 2445 | 26.982 | 8.794 | 50.239 | 1.00 | 18.23 |
| ATOM | 16302 | CB | ASP | 2445 | 27.330 | 7.359 | 49.806 | 1.00 | 20.29 |
| ATOM | 16303 | CG | ASP | 2445 | 28.573 | 6.810 | 50.478 | 1.00 | 22.01 |
| ATOM | 16304 | OD1 | ASP | 2445 | 29.074 | 7.424 | 51.443 | 1.00 | 22.09 |
| ATOM | 16305 | OD2 | ASP | 2445 | 29.039 | 5.741 | 50.030 | 1.00 | 23.26 |
| ATOM | 16306 | C | ASP | 2445 | 26.531 | 8.835 | 51.693 | 1.00 | 17.90 |
| ATOM | 16307 | O | ASP | 2445 | 25.646 | 8.079 | 52.095 | 1.00 | 12.82 |
| ATOM | 16308 | N | SER | 2446 | 27.136 | 9.717 | 52.493 | 1.00 | 16.02 |
| ATOM | 16309 | CA | SER | 2446 | 26.710 | 9.842 | 53.884 | 1.00 | 14.42 |
| ATOM | 16310 | CB | SER | 2446 | 27.556 | 10.877 | 54.632 | 1.00 | 17.31 |
| ATOM | 16311 | OG | SER | 2446 | 27.690 | 12.075 | 53.886 | 1.00 | 19.20 |
| ATOM | 16312 | C | SER | 2446 | 25.260 | 10.294 | 53.838 | 1.00 | 13.92 |
| ATOM | 16313 | O | SER | 2446 | 24.510 | 10.114 | 54.799 | 1.00 | 15.62 |
| ATOM | 16314 | N | LEU | 2447 | 24.879 | 10.885 | 52.706 | 1.00 | 13.83 |
| ATOM | 16315 | CA | LEU | 2447 | 23.511 | 11.366 | 52.490 | 1.00 | 15.03 |
| ATOM | 16316 | CB | LEU | 2447 | 23.383 | 12.005 | 51.095 | 1.00 | 13.29 |
| ATOM | 16317 | CG | LEU | 2447 | 23.435 | 11.139 | 49.828 | 1.00 | 14.14 |
| ATOM | 16318 | CD1 | LEU | 2447 | 22.038 | 10.529 | 49.557 | 1.00 | 14.78 |
| ATOM | 16319 | CD2 | LEU | 2447 | 23.856 | 11.983 | 48.647 | 1.00 | 14.51 |
| ATOM | 16320 | C | LEU | 2447 | 22.505 | 10.232 | 52.636 | 1.00 | 16.55 |
| ATOM | 16321 | O | LEU | 2447 | 21.325 | 10.464 | 52.923 | 1.00 | 16.84 |
| ATOM | 16322 | N | GLY | 2448 | 22.974 | 9.003 | 52.433 | 1.00 | 14.34 |
| ATOM | 16323 | CA | GLY | 2448 | 22.092 | 7.856 | 52.549 | 1.00 | 16.03 |
| ATOM | 16324 | C | GLY | 2448 | 21.544 | 7.753 | 53.952 | 1.00 | 16.17 |
| ATOM | 16325 | O | GLY | 2448 | 20.468 | 7.212 | 54.170 | 1.00 | 14.30 |
| ATOM | 16326 | N | MET | 2449 | 22.290 | 8.287 | 54.910 | 1.00 | 16.60 |
| ATOM | 16327 | CA | MET | 2449 | 21.872 | 8.245 | 56.292 | 1.00 | 16.34 |
| ATOM | 16328 | CB | MET | 2449 | 23.056 | 7.814 | 57.157 | 1.00 | 19.82 |
| ATOM | 16329 | CG | MET | 2449 | 23.644 | 6.484 | 56.710 | 1.00 | 21.64 |
| ATOM | 16330 | SD | MET | 2449 | 24.990 | 5.908 | 57.750 | 1.00 | 24.22 |
| ATOM | 16331 | CE | MET | 2449 | 24.077 | 5.356 | 59.175 | 1.00 | 25.30 |
| ATOM | 16332 | C | MET | 2449 | 21.337 | 9.602 | 56.739 | 1.00 | 17.00 |
| ATOM | 16333 | O | MET | 2449 | 20.194 | 9.710 | 57.177 | 1.00 | 17.40 |
| ATOM | 16334 | N | THR | 2450 | 22.158 | 10.636 | 56.599 | 1.00 | 16.35 |
| ATOM | 16335 | CA | THR | 2450 | 21.774 | 11.980 | 57.007 | 1.00 | 18.67 |
| ATOM | 16336 | CB | THR | 2450 | 22.988 | 12.932 | 56.910 | 1.00 | 20.10 |
| ATOM | 16337 | OG1 | THR | 2450 | 22.658 | 14.201 | 57.489 | 1.00 | 27.83 |
| ATOM | 16338 | CG2 | THR | 2450 | 23.394 | 13.137 | 55.468 | 1.00 | 22.03 |
| ATOM | 16339 | C | THR | 2450 | 20.588 | 12.575 | 56.235 | 1.00 | 18.18 |
| ATOM | 16340 | O | THR | 2450 | 19.764 | 13.287 | 56.808 | 1.00 | 16.73 |
| ATOM | 16341 | N | VAL | 2451 | 20.487 | 12.277 | 54.943 | 1.00 | 16.56 |
| ATOM | 16342 | CA | VAL | 2451 | 19.388 | 12.809 | 54.138 | 1.00 | 15.70 |
| ATOM | 16343 | CB | VAL | 2451 | 19.904 | 13.319 | 52.771 | 1.00 | 16.21 |
| ATOM | 16344 | CG1 | VAL | 2451 | 18.737 | 13.687 | 51.857 | 1.00 | 18.82 |
| ATOM | 16345 | CG2 | VAL | 2451 | 20.794 | 14.531 | 52.983 | 1.00 | 18.81 |
| ATOM | 16346 | C | VAL | 2451 | 18.245 | 11.820 | 53.906 | 1.00 | 15.14 |
| ATOM | 16347 | O | VAL | 2451 | 17.073 | 12.131 | 54.168 | 1.00 | 13.37 |
| ATOM | 16348 | N | GLN | 2452 | 18.580 | 10.631 | 53.415 | 1.00 | 14.84 |
| ATOM | 16349 | CA | GLN | 2452 | 17.573 | 9.605 | 53.135 | 1.00 | 15.81 |
| ATOM | 16350 | CB | GLN | 2452 | 18.130 | 8.588 | 52.137 | 1.00 | 11.46 |
| ATOM | 16351 | CG | GLN | 2452 | 18.666 | 9.217 | 50.868 | 1.00 | 14.21 |
| ATOM | 16352 | CD | GLN | 2452 | 19.187 | 8.181 | 49.893 | 1.00 | 10.70 |
| ATOM | 16353 | OE1 | GLN | 2452 | 19.456 | 7.044 | 50.279 | 1.00 | 14.39 |
| ATOM | 16354 | NE2 | GLN | 2452 | 19.335 | 8.568 | 48.627 | 1.00 | 12.31 |
| ATOM | 16355 | C | GLN | 2452 | 17.071 | 8.883 | 54.376 | 1.00 | 15.31 |
| ATOM | 16356 | O | GLN | 2452 | 15.931 | 8.424 | 54.412 | 1.00 | 16.95 |
| ATOM | 16357 | N | GLY | 2453 | 17.919 | 8.760 | 55.392 | 1.00 | 16.51 |
| ATOM | 16358 | CA | GLY | 2453 | 17.480 | 8.111 | 56.619 | 1.00 | 17.32 |
| ATOM | 16359 | C | GLY | 2453 | 17.695 | 6.616 | 56.735 | 1.00 | 16.27 |
| ATOM | 16360 | O | GLY | 2453 | 17.069 | 5.961 | 57.567 | 1.00 | 15.67 |
| ATOM | 16361 | N | HIS | 2454 | 18.576 | 6.070 | 55.903 | 1.00 | 17.26 |
| ATOM | 16362 | CA | HIS | 2454 | 18.878 | 4.638 | 55.945 | 1.00 | 16.67 |
| ATOM | 16363 | CB | HIS | 2454 | 19.485 | 4.188 | 54.616 | 1.00 | 16.80 |
| ATOM | 16364 | CG | HIS | 2454 | 18.521 | 4.210 | 53.477 | 1.00 | 17.61 |
| ATOM | 16365 | CD2 | HIS | 2454 | 18.472 | 4.977 | 52.363 | 1.00 | 17.85 |
| ATOM | 16366 | ND1 | HIS | 2454 | 17.454 | 3.344 | 53.396 | 1.00 | 16.86 |
| ATOM | 16367 | CE1 | HIS | 2454 | 16.790 | 3.573 | 52.278 | 1.00 | 19.45 |
| ATOM | 16368 | NE2 | HIS | 2454 | 17.387 | 4.559 | 51.633 | 1.00 | 18.52 |
| ATOM | 16369 | C | HIS | 2454 | 19.873 | 4.349 | 57.062 | 1.00 | 17.34 |
| ATOM | 16370 | O | HIS | 2454 | 20.558 | 5.255 | 57.533 | 1.00 | 18.36 |
| ATOM | 16371 | N | ASP | 2455 | 19.945 | 3.084 | 57.475 | 1.00 | 18.16 |
| ATOM | 16372 | CA | ASP | 2455 | 20.858 | 2.646 | 58.531 | 1.00 | 21.37 |
| ATOM | 16373 | CB | ASP | 2455 | 20.435 | 1.270 | 59.074 | 1.00 | 25.85 |
| ATOM | 16374 | CG | ASP | 2455 | 20.380 | 0.193 | 57.991 | 1.00 | 30.92 |
| ATOM | 16375 | OD1 | ASP | 2455 | 21.371 | 0.020 | 57.251 | 1.00 | 33.11 |
| ATOM | 16376 | OD2 | ASP | 2455 | 19.344 | −0.496 | 57.883 | 1.00 | 35.21 |
| ATOM | 16377 | C | ASP | 2455 | 22.315 | 2.561 | 58.061 | 1.00 | 20.40 |
| ATOM | 16378 | O | ASP | 2455 | 23.214 | 2.291 | 58.857 | 1.00 | 22.38 |
| ATOM | 16379 | N | SER | 2456 | 22.542 | 2.782 | 56.775 | 1.00 | 19.74 |
| ATOM | 16380 | CA | SER | 2456 | 23.885 | 2.725 | 56.216 | 1.00 | 19.12 |
| ATOM | 16381 | CB | SER | 2456 | 24.308 | 1.266 | 55.985 | 1.00 | 20.10 |
| ATOM | 16382 | OG | SER | 2456 | 23.643 | 0.697 | 54.870 | 1.00 | 21.89 |
| ATOM | 16383 | C | SER | 2456 | 23.918 | 3.497 | 54.905 | 1.00 | 17.48 |
| ATOM | 16384 | O | SER | 2456 | 22.893 | 3.985 | 54.440 | 1.00 | 17.06 |
| ATOM | 16385 | N | THR | 2457 | 25.101 | 3.616 | 54.312 | 1.00 | 14.78 |
| ATOM | 16386 | CA | THR | 2457 | 25.245 | 4.342 | 53.057 | 1.00 | 13.35 |
| ATOM | 16387 | CB | THR | 2457 | 26.641 | 4.990 | 52.951 | 1.00 | 14.64 |
| ATOM | 16388 | OG1 | THR | 2457 | 27.623 | 3.973 | 52.751 | 1.00 | 14.31 |
| ATOM | 16389 | CG2 | THR | 2457 | 26.984 | 5.744 | 54.237 | 1.00 | 15.15 |
| ATOM | 16390 | C | THR | 2457 | 25.050 | 3.469 | 51.816 | 1.00 | 12.59 |
| ATOM | 16391 | O | THR | 2457 | 24.984 | 3.980 | 50.700 | 1.00 | 14.58 |
| ATOM | 16392 | N | LEU | 2458 | 24.977 | 2.158 | 52.008 | 1.00 | 13.46 |
| ATOM | 16393 | CA | LEU | 2458 | 24.836 | 1.230 | 50.880 | 1.00 | 15.15 |
| ATOM | 16394 | CB | LEU | 2458 | 24.789 | −0.210 | 51.399 | 1.00 | 16.30 |
| ATOM | 16395 | CG | LEU | 2458 | 26.150 | −0.857 | 51.711 | 1.00 | 18.84 |
| ATOM | 16396 | CD1 | LEU | 2458 | 26.834 | −0.141 | 52.881 | 1.00 | 18.45 |
| ATOM | 16397 | CD2 | LEU | 2458 | 25.933 | −2.323 | 52.036 | 1.00 | 19.18 |
| ATOM | 16398 | C | LEU | 2458 | 23.682 | 1.454 | 49.894 | 1.00 | 16.48 |
| ATOM | 16399 | O | LEU | 2458 | 23.853 | 1.265 | 48.690 | 1.00 | 16.20 |
| ATOM | 16400 | N | PRO | 2459 | 22.496 | 1.843 | 50.388 | 1.00 | 17.41 |
| ATOM | 16401 | CD | PRO | 2459 | 22.096 | 1.948 | 51.801 | 1.00 | 17.56 |
| ATOM | 16402 | CA | PRO | 2459 | 21.352 | 2.073 | 49.494 | 1.00 | 16.65 |
| ATOM | 16403 | CB | PRO | 2459 | 20.189 | 2.296 | 50.468 | 1.00 | 19.49 |
| ATOM | 16404 | CG | PRO | 2459 | 20.870 | 2.806 | 51.710 | 1.00 | 23.52 |
| ATOM | 16405 | C | PRO | 2459 | 21.524 | 3.224 | 48.504 | 1.00 | 16.06 |
| ATOM | 16406 | O | PRO | 2459 | 20.800 | 3.311 | 47.511 | 1.00 | 13.44 |
| ATOM | 16407 | N | VAL | 2460 | 22.486 | 4.105 | 48.766 | 1.00 | 13.48 |
| ATOM | 16408 | CA | VAL | 2460 | 22.725 | 5.239 | 47.882 | 1.00 | 14.40 |
| ATOM | 16409 | CB | VAL | 2460 | 23.795 | 6.192 | 48.450 | 1.00 | 14.61 |
| ATOM | 16410 | CG1 | VAL | 2460 | 24.065 | 7.299 | 47.458 | 1.00 | 14.36 |
| ATOM | 16411 | CG2 | VAL | 2460 | 23.329 | 6.762 | 49.780 | 1.00 | 13.84 |
| ATOM | 16412 | C | VAL | 2460 | 23.201 | 4.789 | 46.511 | 1.00 | 15.33 |
| ATOM | 16413 | O | VAL | 2460 | 24.202 | 4.080 | 46.397 | 1.00 | 14.73 |
| ATOM | 16414 | N | THR | 2461 | 22.495 | 5.195 | 45.466 | 1.00 | 16.85 |
| ATOM | 16415 | CA | THR | 2461 | 22.921 | 4.803 | 44.139 | 1.00 | 19.10 |
| ATOM | 16416 | CB | THR | 2461 | 21.798 | 3.984 | 43.403 | 1.00 | 24.26 |
| ATOM | 16417 | OG1 | THR | 2461 | 20.513 | 4.292 | 43.952 | 1.00 | 25.02 |
| ATOM | 16418 | CG2 | THR | 2461 | 22.022 | 2.478 | 43.616 | 1.00 | 25.84 |
| ATOM | 16419 | C | THR | 2461 | 23.450 | 6.011 | 43.351 | 1.00 | 18.53 |
| ATOM | 16420 | O | THR | 2461 | 23.338 | 7.153 | 43.802 | 1.00 | 17.30 |
| ATOM | 16421 | N | VAL | 2462 | 24.074 | 5.758 | 42.207 | 1.00 | 16.22 |
| ATOM | 16422 | CA | VAL | 2462 | 24.651 | 6.823 | 41.392 | 1.00 | 16.40 |
| ATOM | 16423 | CB | VAL | 2462 | 25.214 | 6.253 | 40.072 | 1.00 | 15.77 |
| ATOM | 16424 | CG1 | VAL | 2462 | 25.794 | 7.369 | 39.219 | 1.00 | 15.65 |
| ATOM | 16425 | CG2 | VAL | 2462 | 26.275 | 5.213 | 40.385 | 1.00 | 18.55 |
| ATOM | 16426 | C | VAL | 2462 | 23.679 | 7.951 | 41.057 | 1.00 | 16.36 |
| ATOM | 16427 | O | VAL | 2462 | 24.061 | 9.127 | 41.020 | 1.00 | 16.32 |
| ATOM | 16428 | N | ALA | 2463 | 22.425 | 7.594 | 40.804 | 1.00 | 14.77 |
| ATOM | 16429 | CA | ALA | 2463 | 21.417 | 8.586 | 40.475 | 1.00 | 15.24 |
| ATOM | 16430 | CB | ALA | 2463 | 20.100 | 7.900 | 40.118 | 1.00 | 17.40 |
| ATOM | 16431 | C | ALA | 2463 | 21.211 | 9.540 | 41.647 | 1.00 | 15.53 |
| ATOM | 16432 | O | ALA | 2463 | 20.998 | 10.737 | 41.448 | 1.00 | 17.50 |
| ATOM | 16433 | N | ASP | 2464 | 21.260 | 9.009 | 42.864 | 1.00 | 15.85 |
| ATOM | 16434 | CA | ASP | 2464 | 21.096 | 9.837 | 44.051 | 1.00 | 15.44 |
| ATOM | 16435 | CB | ASP | 2464 | 21.161 | 8.999 | 45.338 | 1.00 | 15.36 |
| ATOM | 16436 | CG | ASP | 2464 | 19.964 | 8.076 | 45.514 | 1.00 | 16.71 |
| ATOM | 16437 | OD1 | ASP | 2464 | 18.885 | 8.353 | 44.930 | 1.00 | 13.53 |
| ATOM | 16438 | OD2 | ASP | 2464 | 20.110 | 7.088 | 46.266 | 1.00 | 15.10 |
| ATOM | 16439 | C | ASP | 2464 | 22.229 | 10.859 | 44.078 | 1.00 | 15.31 |
| ATOM | 16440 | O | ASP | 2464 | 22.000 | 12.058 | 44.249 | 1.00 | 14.61 |
| ATOM | 16441 | N | ILE | 2465 | 23.460 | 10.384 | 43.910 | 1.00 | 14.73 |
| ATOM | 16442 | CA | ILE | 2465 | 24.613 | 11.283 | 43.928 | 1.00 | 14.21 |
| ATOM | 16443 | CB | ILE | 2465 | 25.937 | 10.504 | 43.708 | 1.00 | 14.08 |
| ATOM | 16444 | CG2 | ILE | 2465 | 27.104 | 11.484 | 43.609 | 1.00 | 12.92 |
| ATOM | 16445 | CG1 | ILE | 2465 | 26.159 | 9.511 | 44.861 | 1.00 | 11.78 |
| ATOM | 16446 | CD1 | ILE | 2465 | 26.437 | 10.163 | 46.224 | 1.00 | 16.30 |
| ATOM | 16447 | C | ILE | 2465 | 24.482 | 12.377 | 42.867 | 1.00 | 14.68 |
| ATOM | 16448 | O | ILE | 2465 | 24.675 | 13.556 | 43.159 | 1.00 | 15.78 |
| ATOM | 16449 | N | ALA | 2466 | 24.137 | 11.995 | 41.642 | 1.00 | 13.89 |
| ATOM | 16450 | CA | ALA | 2466 | 23.993 | 12.960 | 40.551 | 1.00 | 14.00 |
| ATOM | 16451 | CB | ALA | 2466 | 23.631 | 12.229 | 39.246 | 1.00 | 13.36 |
| ATOM | 16452 | C | ALA | 2466 | 22.942 | 14.027 | 40.849 | 1.00 | 14.38 |
| ATOM | 16453 | O | ALA | 2466 | 23.082 | 15.179 | 40.445 | 1.00 | 16.13 |
| ATOM | 16454 | N | TYR | 2467 | 21.871 | 13.620 | 41.518 | 1.00 | 15.16 |
| ATOM | 16455 | CA | TYR | 2467 | 20.792 | 14.527 | 41.874 | 1.00 | 14.44 |
| ATOM | 16456 | CB | TYR | 2467 | 19.660 | 13.744 | 42.536 | 1.00 | 15.27 |
| ATOM | 16457 | CG | TYR | 2467 | 18.537 | 14.592 | 43.094 | 1.00 | 16.93 |
| ATOM | 16458 | CD1 | TYR | 2467 | 17.661 | 15.271 | 42.252 | 1.00 | 17.28 |
| ATOM | 16459 | CE1 | TYR | 2467 | 16.603 | 16.016 | 42.765 | 1.00 | 17.30 |
| ATOM | 16460 | CD2 | TYR | 2467 | 18.331 | 14.680 | 44.471 | 1.00 | 17.80 |
| ATOM | 16461 | CE2 | TYR | 2467 | 17.281 | 15.419 | 45.000 | 1.00 | 17.99 |
| ATOM | 16462 | CZ | TYR | 2467 | 16.415 | 16.084 | 44.143 | 1.00 | 19.01 |
| ATOM | 16463 | OH | TYR | 2467 | 15.355 | 16.787 | 44.665 | 1.00 | 16.68 |
| ATOM | 16464 | C | TYR | 2467 | 21.293 | 15.606 | 42.833 | 1.00 | 12.99 |
| ATOM | 16465 | O | TYR | 2467 | 21.094 | 16.799 | 42.605 | 1.00 | 14.73 |
| ATOM | 16466 | N | HIS | 2468 | 21.942 | 15.180 | 43.905 | 1.00 | 13.09 |
| ATOM | 16467 | CA | HIS | 2468 | 22.450 | 16.117 | 44.897 | 1.00 | 14.34 |
| ATOM | 16468 | CB | HIS | 2468 | 22.822 | 15.360 | 46.174 | 1.00 | 13.15 |
| ATOM | 16469 | CG | HIS | 2468 | 21.632 | 14.846 | 46.924 | 1.00 | 17.11 |
| ATOM | 16470 | CD2 | HIS | 2468 | 21.090 | 13.606 | 46.999 | 1.00 | 17.53 |
| ATOM | 16471 | ND1 | HIS | 2468 | 20.797 | 15.671 | 47.649 | 1.00 | 15.01 |
| ATOM | 16472 | CE1 | HIS | 2468 | 19.792 | 14.963 | 48.134 | 1.00 | 17.40 |
| ATOM | 16473 | NE2 | HIS | 2468 | 19.945 | 13.707 | 47.753 | 1.00 | 18.53 |
| ATOM | 16474 | C | HIS | 2468 | 23.626 | 16.936 | 44.378 | 1.00 | 15.33 |
| ATOM | 16475 | O | HIS | 2468 | 23.834 | 18.069 | 44.803 | 1.00 | 14.82 |
| ATOM | 16476 | N | THR | 2469 | 24.391 | 16.366 | 43.452 | 1.00 | 15.33 |
| ATOM | 16477 | CA | THR | 2469 | 25.527 | 17.073 | 42.873 | 1.00 | 16.60 |
| ATOM | 16478 | CB | THR | 2469 | 26.348 | 16.132 | 41.968 | 1.00 | 16.20 |
| ATOM | 16479 | OG1 | THR | 2469 | 27.046 | 15.184 | 42.785 | 1.00 | 17.12 |
| ATOM | 16480 | CG2 | THR | 2469 | 27.352 | 16.919 | 41.134 | 1.00 | 14.14 |
| ATOM | 16481 | C | THR | 2469 | 25.066 | 18.298 | 42.078 | 1.00 | 16.62 |
| ATOM | 16482 | O | THR | 2469 | 25.631 | 19.387 | 42.215 | 1.00 | 18.28 |
| ATOM | 16483 | N | ALA | 2470 | 24.034 | 18.122 | 41.256 | 1.00 | 17.01 |
| ATOM | 16484 | CA | ALA | 2470 | 23.508 | 19.222 | 40.461 | 1.00 | 16.73 |
| ATOM | 16485 | CB | ALA | 2470 | 22.383 | 18.726 | 39.549 | 1.00 | 18.75 |
| ATOM | 16486 | C | ALA | 2470 | 22.992 | 20.320 | 41.391 | 1.00 | 17.94 |
| ATOM | 16487 | O | ALA | 2470 | 23.185 | 21.505 | 41.125 | 1.00 | 18.99 |
| ATOM | 16488 | N | ALA | 2471 | 22.342 | 19.921 | 42.479 | 1.00 | 17.24 |
| ATOM | 16489 | CA | ALA | 2471 | 21.807 | 20.875 | 43.445 | 1.00 | 19.28 |
| ATOM | 16490 | CB | ALA | 2471 | 21.024 | 20.137 | 44.529 | 1.00 | 18.10 |
| ATOM | 16491 | C | ALA | 2471 | 22.921 | 21.705 | 44.083 | 1.00 | 20.30 |
| ATOM | 16492 | O | ALA | 2471 | 22.814 | 22.927 | 44.191 | 1.00 | 22.29 |
| ATOM | 16493 | N | VAL | 2472 | 23.987 | 21.036 | 44.510 | 1.00 | 21.49 |
| ATOM | 16494 | CA | VAL | 2472 | 25.126 | 21.708 | 45.144 | 1.00 | 20.58 |
| ATOM | 16495 | CB | VAL | 2472 | 26.149 | 20.670 | 45.669 | 1.00 | 20.42 |
| ATOM | 16496 | CG1 | VAL | 2472 | 27.441 | 21.359 | 46.104 | 1.00 | 18.63 |
| ATOM | 16497 | CG2 | VAL | 2472 | 25.543 | 19.901 | 46.832 | 1.00 | 17.07 |
| ATOM | 16498 | C | VAL | 2472 | 25.829 | 22.656 | 44.178 | 1.00 | 21.47 |
| ATOM | 16499 | O | VAL | 2472 | 26.220 | 23.763 | 44.552 | 1.00 | 20.66 |
| ATOM | 16500 | N | ARG | 2473 | 26.000 | 22.215 | 42.938 | 1.00 | 20.73 |
| ATOM | 16501 | CA | ARG | 2473 | 26.651 | 23.043 | 41.939 | 1.00 | 20.95 |
| ATOM | 16502 | CB | ARG | 2473 | 26.805 | 22.273 | 40.622 | 1.00 | 21.67 |
| ATOM | 16503 | CG | ARG | 2473 | 27.384 | 23.113 | 39.490 | 1.00 | 23.02 |
| ATOM | 16504 | CD | ARG | 2473 | 28.729 | 23.711 | 39.889 | 1.00 | 23.89 |
| ATOM | 16505 | NE | ARG | 2473 | 29.797 | 22.724 | 39.904 | 1.00 | 23.45 |
| ATOM | 16506 | CZ | ARG | 2473 | 30.932 | 22.858 | 40.584 | 1.00 | 26.20 |
| ATOM | 16507 | NH1 | ARG | 2473 | 31.148 | 23.941 | 41.320 | 1.00 | 24.63 |
| ATOM | 16508 | NH2 | ARG | 2473 | 31.864 | 21.915 | 40.515 | 1.00 | 21.81 |
| ATOM | 16509 | C | ARG | 2473 | 25.857 | 24.326 | 41.699 | 1.00 | 22.12 |
| ATOM | 16510 | O | ARG | 2473 | 26.437 | 25.398 | 41.529 | 1.00 | 23.41 |
| ATOM | 16511 | N | ARG | 2474 | 24.532 | 24.229 | 41.684 | 1.00 | 20.61 |
| ATOM | 16512 | CA | ARG | 2474 | 23.728 | 25.424 | 41.459 | 1.00 | 22.05 |
| ATOM | 16513 | CB | ARG | 2474 | 22.237 | 25.075 | 41.369 | 1.00 | 23.15 |
| ATOM | 16514 | CG | ARG | 2474 | 21.883 | 24.197 | 40.178 | 1.00 | 25.72 |
| ATOM | 16515 | CD | ARG | 2474 | 20.381 | 24.163 | 39.933 | 1.00 | 26.31 |
| ATOM | 16516 | NE | ARG | 2474 | 20.023 | 23.166 | 38.930 | 1.00 | 29.12 |
| ATOM | 16517 | CZ | ARG | 2474 | 19.951 | 21.859 | 39.159 | 1.00 | 30.89 |
| ATOM | 16518 | NH1 | ARG | 2474 | 20.205 | 21.375 | 40.370 | 1.00 | 31.05 |
| ATOM | 16519 | NH2 | ARG | 2474 | 19.638 | 21.030 | 38.168 | 1.00 | 29.85 |
| ATOM | 16520 | C | ARG | 2474 | 23.958 | 26.446 | 42.571 | 1.00 | 21.79 |
| ATOM | 16521 | O | ARG | 2474 | 23.888 | 27.651 | 42.337 | 1.00 | 19.95 |
| ATOM | 16522 | N | GLY | 2475 | 24.249 | 25.959 | 43.772 | 1.00 | 21.36 |
| ATOM | 16523 | CA | GLY | 2475 | 24.487 | 26.853 | 44.889 | 1.00 | 23.25 |
| ATOM | 16524 | C | GLY | 2475 | 25.922 | 27.337 | 44.982 | 1.00 | 23.98 |
| ATOM | 16525 | O | GLY | 2475 | 26.186 | 28.396 | 45.552 | 1.00 | 23.43 |
| ATOM | 16526 | N | ALA | 2476 | 26.849 | 26.568 | 44.418 | 1.00 | 23.91 |
| ATOM | 16527 | CA | ALA | 2476 | 28.264 | 26.920 | 44.447 | 1.00 | 25.38 |
| ATOM | 16528 | CB | ALA | 2476 | 28.948 | 26.219 | 45.616 | 1.00 | 25.54 |
| ATOM | 16529 | C | ALA | 2476 | 28.930 | 26.517 | 43.134 | 1.00 | 26.02 |
| ATOM | 16530 | O | ALA | 2476 | 29.616 | 25.498 | 43.066 | 1.00 | 27.80 |
| ATOM | 16531 | N | PRO | 2477 | 28.741 | 27.319 | 42.074 | 1.00 | 26.85 |
| ATOM | 16532 | CD | PRO | 2477 | 27.952 | 28.563 | 42.035 | 1.00 | 28.05 |
| ATOM | 16533 | CA | PRO | 2477 | 29.322 | 27.038 | 40.757 | 1.00 | 29.05 |
| ATOM | 16534 | CB | PRO | 2477 | 28.607 | 28.035 | 39.853 | 1.00 | 28.77 |
| ATOM | 16535 | CG | PRO | 2477 | 28.429 | 29.204 | 40.748 | 1.00 | 29.67 |
| ATOM | 16536 | C | PRO | 2477 | 30.839 | 27.159 | 40.664 | 1.00 | 29.31 |
| ATOM | 16537 | O | PRO | 2477 | 31.441 | 26.680 | 39.706 | 1.00 | 30.76 |
| ATOM | 16538 | N | ASN | 2478 | 31.453 | 27.788 | 41.661 | 1.00 | 30.39 |
| ATOM | 16539 | CA | ASN | 2478 | 32.897 | 27.974 | 41.655 | 1.00 | 29.62 |
| ATOM | 16540 | CB | ASN | 2478 | 33.229 | 29.453 | 41.881 | 1.00 | 30.92 |
| ATOM | 16541 | CG | ASN | 2478 | 32.571 | 30.360 | 40.858 | 1.00 | 31.62 |
| ATOM | 16542 | OD1 | ASN | 2478 | 32.859 | 30.284 | 39.665 | 1.00 | 33.22 |
| ATOM | 16543 | ND2 | ASN | 2478 | 31.667 | 31.215 | 41.322 | 1.00 | 34.26 |
| ATOM | 16544 | C | ASN | 2478 | 33.616 | 27.128 | 42.695 | 1.00 | 28.50 |
| ATOM | 16545 | O | ASN | 2478 | 34.828 | 27.231 | 42.842 | 1.00 | 29.41 |
| ATOM | 16546 | N | CYS | 2479 | 32.877 | 26.280 | 43.402 | 1.00 | 27.76 |
| ATOM | 16547 | CA | CYS | 2479 | 33.487 | 25.444 | 44.428 | 1.00 | 24.78 |
| ATOM | 16548 | CB | CYS | 2479 | 32.444 | 25.031 | 45.475 | 1.00 | 25.50 |
| ATOM | 16549 | SG | CYS | 2479 | 31.459 | 23.551 | 45.037 | 1.00 | 26.02 |
| ATOM | 16550 | C | CYS | 2479 | 34.122 | 24.188 | 43.845 | 1.00 | 24.27 |
| ATOM | 16551 | O | CYS | 2479 | 33.787 | 23.760 | 42.740 | 1.00 | 23.85 |
| ATOM | 16552 | N | LEU | 2480 | 35.065 | 23.616 | 44.589 | 1.00 | 22.97 |
| ATOM | 16553 | CA | LEU | 2480 | 35.697 | 22.375 | 44.175 | 1.00 | 22.48 |
| ATOM | 16554 | CB | LEU | 2480 | 37.062 | 22.189 | 44.852 | 1.00 | 22.33 |
| ATOM | 16555 | CG | LEU | 2480 | 37.763 | 20.855 | 44.584 | 1.00 | 23.00 |
| ATOM | 16556 | CD1 | LEU | 2480 | 37.990 | 20.684 | 43.095 | 1.00 | 23.31 |
| ATOM | 16557 | CD2 | LEU | 2480 | 39.087 | 20.787 | 45.335 | 1.00 | 22.05 |
| ATOM | 16558 | C | LEU | 2480 | 34.693 | 21.342 | 44.680 | 1.00 | 21.39 |
| ATOM | 16559 | O | LEU | 2480 | 34.571 | 21.114 | 45.884 | 1.00 | 20.80 |
| ATOM | 16560 | N | LEU | 2481 | 33.960 | 20.739 | 43.749 | 1.00 | 20.74 |
| ATOM | 16561 | CA | LEU | 2481 | 32.926 | 19.772 | 44.089 | 1.00 | 19.90 |
| ATOM | 16562 | CB | LEU | 2481 | 31.684 | 20.025 | 43.217 | 1.00 | 21.31 |
| ATOM | 16563 | CG | LEU | 2481 | 30.295 | 19.607 | 43.726 | 1.00 | 24.25 |
| ATOM | 16564 | CD1 | LEU | 2481 | 29.231 | 20.182 | 42.806 | 1.00 | 24.77 |
| ATOM | 16565 | CD2 | LEU | 2481 | 30.184 | 18.094 | 43.805 | 1.00 | 25.65 |
| ATOM | 16566 | C | LEU | 2481 | 33.384 | 18.328 | 43.931 | 1.00 | 20.50 |
| ATOM | 16367 | O | LEU | 2481 | 33.681 | 17.875 | 42.824 | 1.00 | 20.28 |
| ATOM | 16568 | N | LEU | 2482 | 33.451 | 17.613 | 45.046 | 1.00 | 19.62 |
| ATOM | 16569 | CA | LEU | 2482 | 33.844 | 16.211 | 45.030 | 1.00 | 19.59 |
| ATOM | 16570 | CB | LEU | 2482 | 34.776 | 15.889 | 46.198 | 1.00 | 19.36 |
| ATOM | 16571 | CG | LEU | 2482 | 36.249 | 16.304 | 46.107 | 1.00 | 21.46 |
| ATOM | 16572 | CD1 | LEU | 2482 | 36.385 | 17.805 | 45.908 | 1.00 | 24.55 |
| ATOM | 16573 | CD2 | LEU | 2482 | 36.947 | 15.854 | 47.372 | 1.00 | 19.98 |
| ATOM | 16574 | C | LEU | 2482 | 32.569 | 15.397 | 45.178 | 1.00 | 19.82 |
| ATOM | 16575 | O | LEU | 2482 | 31.701 | 15.752 | 45.972 | 1.00 | 21.78 |
| ATOM | 16576 | N | ALA | 2483 | 32.432 | 14.323 | 44.411 | 1.00 | 16.25 |
| ATOM | 16577 | CA | ALA | 2483 | 31.240 | 13.493 | 44.541 | 1.00 | 16.04 |
| ATOM | 16578 | CB | ALA | 2483 | 30.348 | 13.636 | 43.323 | 1.00 | 15.47 |
| ATOM | 16579 | C | ALA | 2483 | 31.678 | 12.051 | 44.714 | 1.00 | 14.99 |
| ATOM | 16580 | O | ALA | 2483 | 32.594 | 11.591 | 44.039 | 1.00 | 14.48 |
| ATOM | 16581 | N | ASP | 2484 | 31.033 | 11.346 | 45.636 | 1.00 | 14.00 |
| ATOM | 16582 | CA | ASP | 2484 | 31.363 | 9.952 | 45.907 | 1.00 | 16.62 |
| ATOM | 16583 | CB | ASP | 2484 | 30.819 | 9.508 | 47.265 | 1.00 | 18.12 |
| ATOM | 16584 | CG | ASP | 2484 | 31.751 | 9.800 | 48.420 | 1.00 | 21.09 |
| ATOM | 16585 | OD1 | ASP | 2484 | 32.952 | 10.027 | 48.201 | 1.00 | 23.51 |
| ATOM | 16586 | OD2 | ASP | 2484 | 31.259 | 9.765 | 49.567 | 1.00 | 25.92 |
| ATOM | 16587 | C | ASP | 2484 | 30.751 | 8.990 | 44.906 | 1.00 | 16.88 |
| ATOM | 16588 | O | ASP | 2484 | 29.679 | 9.252 | 44.362 | 1.00 | 17.02 |
| ATOM | 16589 | N | LEU | 2485 | 31.437 | 7.878 | 44.664 | 1.00 | 16.05 |
| ATOM | 16590 | CA | LEU | 2485 | 30.856 | 6.828 | 43.837 | 1.00 | 16.81 |
| ATOM | 16591 | CB | LEU | 2485 | 31.887 | 6.120 | 42.968 | 1.00 | 17.36 |
| ATOM | 16592 | CG | LEU | 2485 | 32.362 | 6.967 | 41.791 | 1.00 | 18.54 |
| ATOM | 16593 | CD1 | LEU | 2485 | 33.158 | 6.088 | 40.829 | 1.00 | 19.06 |
| ATOM | 16594 | CD2 | LEU | 2485 | 31.157 | 7.568 | 41.072 | 1.00 | 18.74 |
| ATOM | 16595 | C | LEU | 2485 | 30.367 | 5.900 | 44.936 | 1.00 | 15.90 |
| ATOM | 16596 | O | LEU | 2485 | 31.145 | 5.447 | 45.773 | 1.00 | 16.45 |
| ATOM | 16597 | N | PRO | 2486 | 29.057 | 5.638 | 44.970 | 1.00 | 17.02 |
| ATOM | 16598 | CD | PRO | 2486 | 28.055 | 6.209 | 44.052 | 1.00 | 16.98 |
| ATOM | 16599 | CA | PRO | 2486 | 28.427 | 4.777 | 45.968 | 1.00 | 15.60 |
| ATOM | 16600 | CB | PRO | 2486 | 26.937 | 5.042 | 45.750 | 1.00 | 15.78 |
| ATOM | 16601 | CG | PRO | 2486 | 26.860 | 5.312 | 44.276 | 1.00 | 16.02 |
| ATOM | 16602 | C | PRO | 2486 | 28.778 | 3.287 | 45.908 | 1.00 | 14.53 |
| ATOM | 16603 | O | PRO | 2486 | 29.499 | 2.822 | 45.025 | 1.00 | 14.20 |
| ATOM | 16604 | N | PHE | 2487 | 28.267 | 2.559 | 46.890 | 1.00 | 14.83 |
| ATOM | 16605 | CA | PHE | 2487 | 28.469 | 1.125 | 47.011 | 1.00 | 15.84 |
| ATOM | 16606 | CB | PHE | 2487 | 27.501 | 0.583 | 48.074 | 1.00 | 15.61 |
| ATOM | 16607 | CG | PHE | 2487 | 27.356 | −0.919 | 48.083 | 1.00 | 17.92 |
| ATOM | 16608 | CD1 | PHE | 2487 | 28.450 | −1.746 | 48.328 | 1.00 | 16.77 |
| ATOM | 16609 | CD2 | PHE | 2487 | 26.106 | −1.504 | 47.874 | 1.00 | 15.14 |
| ATOM | 16610 | CE1 | PHE | 2487 | 28.302 | −3.131 | 48.369 | 1.00 | 18.81 |
| ATOM | 16611 | CE2 | PHE | 2487 | 25.948 | −2.887 | 47.913 | 1.00 | 18.55 |
| ATOM | 16612 | CZ | PHE | 2487 | 27.050 | −3.706 | 48.160 | 1.00 | 17.93 |
| ATOM | 16613 | C | PHE | 2487 | 28.283 | 0.389 | 45.676 | 1.00 | 16.50 |
| ATOM | 16614 | O | PHE | 2487 | 27.257 | 0.535 | 45.007 | 1.00 | 15.22 |
| ATOM | 16615 | N | MET | 2488 | 29.297 | −0.390 | 45.302 | 1.00 | 16.30 |
| ATOM | 16616 | CA | MET | 2488 | 29.305 | −1.193 | 44.077 | 1.00 | 16.43 |
| ATOM | 16617 | CB | MET | 2488 | 28.265 | −2.320 | 44.174 | 1.00 | 17.44 |
| ATOM | 16618 | CG | MET | 2488 | 28.596 | −3.554 | 43.353 | 1.00 | 17.50 |
| ATOM | 16619 | SD | MET | 2488 | 30.158 | −4.247 | 43.921 | 1.00 | 15.86 |
| ATOM | 16620 | CE | MET | 2488 | 29.646 | −5.320 | 45.243 | 1.00 | 15.24 |
| ATOM | 16621 | C | MET | 2488 | 29.084 | −0.384 | 42.804 | 1.00 | 18.13 |
| ATOM | 16622 | O | MET | 2488 | 28.516 | −0.882 | 41.827 | 1.00 | 17.92 |
| ATOM | 16623 | N | ALA | 2489 | 29.547 | 0.862 | 42.810 | 1.00 | 15.73 |
| ATOM | 16624 | CA | ALA | 2489 | 29.417 | 1.732 | 41.643 | 1.00 | 16.90 |
| ATOM | 16625 | CB | ALA | 2489 | 28.963 | 3.133 | 42.074 | 1.00 | 15.45 |
| ATOM | 16626 | C | ALA | 2489 | 30.750 | 1.807 | 40.891 | 1.00 | 14.87 |
| ATOM | 16627 | O | ALA | 2489 | 30.887 | 2.539 | 39.916 | 1.00 | 16.09 |
| ATOM | 16628 | N | TYR | 2490 | 31.731 | 1.040 | 41.352 | 1.00 | 16.77 |
| ATOM | 16629 | CA | TYR | 2490 | 33.043 | 1.000 | 40.705 | 1.00 | 16.61 |
| ATOM | 16630 | CB | TYR | 2490 | 33.978 | 2.071 | 41.293 | 1.00 | 17.08 |
| ATOM | 16631 | CG | TYR | 2490 | 34.015 | 2.131 | 42.812 | 1.00 | 19.16 |
| ATOM | 16632 | CD1 | TYR | 2490 | 35.039 | 1.521 | 43.538 | 1.00 | 18.38 |
| ATOM | 16633 | CE1 | TYR | 2490 | 35.062 | 1.572 | 44.939 | 1.00 | 18.54 |
| ATOM | 16634 | CD2 | TYR | 2490 | 33.011 | 2.795 | 43.519 | 1.00 | 20.42 |
| ATOM | 16635 | CE2 | TYR | 2490 | 33.017 | 2.855 | 44.907 | 1.00 | 20.22 |
| ATOM | 16636 | CZ | TYR | 2490 | 34.042 | 2.244 | 45.613 | 1.00 | 21.20 |
| ATOM | 16637 | OH | TYR | 2490 | 34.038 | 2.326 | 46.990 | 1.00 | 19.10 |
| ATOM | 16638 | C | TYR | 2490 | 33.652 | −0.390 | 40.875 | 1.00 | 16.36 |
| ATOM | 16639 | O | TYR | 2490 | 34.842 | −0.532 | 41.154 | 1.00 | 15.49 |
| ATOM | 16640 | N | ALA | 2491 | 32.821 | −1.413 | 40.687 | 1.00 | 15.34 |
| ATOM | 16641 | CA | ALA | 2491 | 33.240 | −2.806 | 40.845 | 1.00 | 16.21 |
| ATOM | 16642 | CB | ALA | 2491 | 32.016 | −3.716 | 40.865 | 1.00 | 16.00 |
| ATOM | 16643 | C | ALA | 2491 | 34.218 | −3.256 | 39.765 | 1.00 | 16.41 |
| ATOM | 16644 | O | ALA | 2491 | 34.948 | −4.229 | 39.945 | 1.00 | 16.54 |
| ATOM | 16645 | N | THR | 2492 | 34.196 | −2.571 | 38.631 | 1.00 | 16.89 |
| ATOM | 16646 | CA | THR | 2492 | 35.127 | −2.863 | 37.544 | 1.00 | 18.84 |
| ATOM | 16647 | CB | THR | 2492 | 34.507 | −3.747 | 36.423 | 1.00 | 17.42 |
| ATOM | 16648 | OG1 | THR | 2492 | 33.628 | −2.948 | 35.619 | 1.00 | 19.01 |
| ATOM | 16649 | CG2 | THR | 2492 | 33.753 | −4.919 | 37.015 | 1.00 | 16.56 |
| ATOM | 16650 | C | THR | 2492 | 35.476 | −1.517 | 36.939 | 1.00 | 19.20 |
| ATOM | 16651 | O | THR | 2492 | 34.705 | −0.563 | 37.040 | 1.00 | 17.78 |
| ATOM | 16652 | N | PRO | 2493 | 36.655 | −1.416 | 36.316 | 1.00 | 20.58 |
| ATOM | 16653 | CD | PRO | 2493 | 37.710 | −2.440 | 36.217 | 1.00 | 22.28 |
| ATOM | 16654 | CA | PRO | 2493 | 37.080 | −0.163 | 35.696 | 1.00 | 21.26 |
| ATOM | 16655 | CB | PRO | 2493 | 38.359 | −0.561 | 34.968 | 1.00 | 22.19 |
| ATOM | 16656 | CG | PRO | 2493 | 38.925 | −1.615 | 35.861 | 1.00 | 22.73 |
| ATOM | 16657 | C | PRO | 2493 | 36.031 | 0.379 | 34.737 | 1.00 | 21.17 |
| ATOM | 16658 | O | PRO | 2493 | 35.718 | 1.567 | 34.752 | 1.00 | 20.07 |
| ATOM | 16659 | N | GLU | 2494 | 35.477 | −0.497 | 33.906 | 1.00 | 21.51 |
| ATOM | 16660 | CA | GLU | 2494 | 34.485 | −0.062 | 32.934 | 1.00 | 22.98 |
| ATOM | 16661 | CB | GLU | 2494 | 34.027 | −1.249 | 32.078 | 1.00 | 27.93 |
| ATOM | 16662 | CG | GLU | 2494 | 33.193 | −0.865 | 30.862 | 1.00 | 35.30 |
| ATOM | 16663 | CD | GLU | 2494 | 33.127 | −1.979 | 29.825 | 1.00 | 39.26 |
| ATOM | 16664 | OE1 | GLU | 2494 | 32.813 | −3.129 | 30.207 | 1.00 | 40.58 |
| ATOM | 16665 | OE2 | GLU | 2494 | 33.381 | −1.701 | 28.629 | 1.00 | 41.62 |
| ATOM | 16666 | C | GLU | 2494 | 33.296 | 0.596 | 33.629 | 1.00 | 21.02 |
| ATOM | 16667 | O | GLU | 2494 | 32.867 | 1.674 | 33.236 | 1.00 | 20.30 |
| ATOM | 16668 | N | GLN | 2495 | 32.768 | −0.043 | 34.663 | 1.00 | 19.92 |
| ATOM | 16669 | CA | GLN | 2495 | 31.638 | 0.536 | 35.377 | 1.00 | 21.90 |
| ATOM | 16670 | CB | GLN | 2495 | 31.103 | −0.446 | 36.411 | 1.00 | 25.01 |
| ATOM | 16671 | CG | GLN | 2495 | 30.627 | −1.747 | 35.803 | 1.00 | 32.47 |
| ATOM | 16672 | CD | GLN | 2495 | 29.946 | −2.626 | 36.814 | 1.00 | 34.62 |
| ATOM | 16673 | OE1 | GLN | 2495 | 28.836 | −2.332 | 37.259 | 1.00 | 37.69 |
| ATOM | 16674 | NE2 | GLN | 2495 | 30.614 | −3.707 | 37.202 | 1.00 | 39.43 |
| ATOM | 16675 | C | GLN | 2495 | 32.044 | 1.835 | 36.056 | 1.00 | 18.71 |
| ATOM | 16676 | O | GLN | 2495 | 31.269 | 2.788 | 36.101 | 1.00 | 16.24 |
| ATOM | 16677 | N | ALA | 2496 | 33.263 | 1.870 | 36.583 | 1.00 | 18.87 |
| ATOM | 16678 | CA | ALA | 2496 | 33.756 | 3.073 | 37.236 | 1.00 | 18.29 |
| ATOM | 16679 | CB | ALA | 2496 | 35.161 | 2.842 | 37.789 | 1.00 | 19.37 |
| ATOM | 16680 | C | ALA | 2496 | 33.758 | 4.249 | 36.258 | 1.00 | 17.83 |
| ATOM | 16681 | O | ALA | 2496 | 33.373 | 5.355 | 36.626 | 1.00 | 17.54 |
| ATOM | 16682 | N | PHE | 2497 | 34.181 | 4.018 | 35.014 | 1.00 | 17.22 |
| ATOM | 16683 | CA | PHE | 2497 | 34.215 | 5.104 | 34.033 | 1.00 | 16.32 |
| ATOM | 16684 | CB | PHE | 2497 | 34.777 | 4.639 | 32.680 | 1.00 | 15.77 |
| ATOM | 16685 | CG | PHE | 2497 | 36.109 | 3.944 | 32.763 | 1.00 | 16.08 |
| ATOM | 16686 | CD1 | PHE | 2497 | 37.023 | 4.262 | 33.763 | 1.00 | 17.05 |
| ATOM | 16687 | CD2 | PHE | 2497 | 36.453 | 2.976 | 31.822 | 1.00 | 19.05 |
| ATOM | 16688 | CE1 | PHE | 2497 | 38.264 | 3.626 | 33.831 | 1.00 | 18.78 |
| ATOM | 16689 | CE2 | PHE | 2497 | 37.686 | 2.334 | 31.876 | 1.00 | 18.30 |
| ATOM | 16690 | CZ | PHE | 2497 | 38.594 | 2.660 | 32.885 | 1.00 | 19.48 |
| ATOM | 16691 | C | PHE | 2497 | 32.817 | 5.659 | 33.796 | 1.00 | 16.99 |
| ATOM | 16692 | O | PHE | 2497 | 32.616 | 6.872 | 33.761 | 1.00 | 15.02 |
| ATOM | 16693 | N | GLU | 2498 | 31.860 | 4.753 | 33.624 | 1.00 | 17.61 |
| ATOM | 16694 | CA | GLU | 2498 | 30.474 | 5.121 | 33.364 | 1.00 | 19.06 |
| ATOM | 16695 | CB | GLU | 2498 | 29.635 | 3.854 | 33.162 | 1.00 | 22.66 |
| ATOM | 16696 | CG | GLU | 2498 | 28.212 | 4.090 | 32.676 | 1.00 | 28.82 |
| ATOM | 16697 | CD | GLU | 2498 | 28.165 | 4.785 | 31.322 | 1.00 | 33.26 |
| ATOM | 16698 | OE1 | GLU | 2498 | 29.130 | 4.638 | 30.536 | 1.00 | 33.89 |
| ATOM | 16699 | OE2 | GLU | 2498 | 27.156 | 5.469 | 31.038 | 1.00 | 35.72 |
| ATOM | 16700 | C | GLU | 2498 | 29.880 | 5.947 | 34.499 | 1.00 | 18.47 |
| ATOM | 16701 | O | GLU | 2498 | 29.368 | 7.049 | 34.281 | 1.00 | 19.38 |
| ATOM | 16702 | N | ASN | 2499 | 29.954 | 5.413 | 35.711 | 1.00 | 18.25 |
| ATOM | 16703 | CA | ASN | 2499 | 29.392 | 6.105 | 36.869 | 1.00 | 19.15 |
| ATOM | 16704 | CB | ASN | 2499 | 29.335 | 5.157 | 38.070 | 1.00 | 19.68 |
| ATOM | 16705 | CG | ASN | 2499 | 28.385 | 3.997 | 37.839 | 1.00 | 19.41 |
| ATOM | 16706 | OD1 | ASN | 2499 | 27.309 | 4.178 | 37.269 | 1.00 | 21.49 |
| ATOM | 16707 | ND2 | ASN | 2499 | 28.767 | 2.811 | 38.283 | 1.00 | 18.16 |
| ATOM | 16708 | C | ASN | 2499 | 30.125 | 7.397 | 37.225 | 1.00 | 19.51 |
| ATOM | 16709 | O | ASN | 2499 | 29.513 | 8.347 | 37.723 | 1.00 | 18.86 |
| ATOM | 16710 | N | ALA | 2500 | 31.429 | 7.440 | 36.965 | 1.00 | 20.06 |
| ATOM | 16711 | CA | ALA | 2500 | 32.207 | 8.641 | 37.239 | 1.00 | 19.16 |
| ATOM | 16712 | CB | ALA | 2500 | 33.694 | 8.367 | 37.043 | 1.00 | 19.94 |
| ATOM | 16713 | C | ALA | 2500 | 31.748 | 9.734 | 36.284 | 1.00 | 19.10 |
| ATOM | 16714 | O | ALA | 2500 | 31.472 | 10.858 | 36.695 | 1.00 | 18.94 |
| ATOM | 16715 | N | ALA | 2501 | 31.652 | 9.389 | 35.004 | 1.00 | 17.77 |
| ATOM | 16716 | CA | ALA | 2501 | 31.229 | 10.339 | 33.991 | 1.00 | 16.26 |
| ATOM | 16717 | CB | ALA | 2501 | 31.220 | 9.670 | 32.608 | 1.00 | 16.19 |
| ATOM | 16718 | C | ALA | 2501 | 29.850 | 10.905 | 34.303 | 1.00 | 15.89 |
| ATOM | 16719 | O | ALA | 2501 | 29.592 | 12.070 | 34.028 | 1.00 | 18.58 |
| ATOM | 16720 | N | THR | 2502 | 28.963 | 10.081 | 34.862 | 1.00 | 16.35 |
| ATOM | 16721 | CA | THR | 2502 | 27.605 | 10.524 | 35.200 | 1.00 | 16.80 |
| ATOM | 16722 | CB | THR | 2502 | 26.731 | 9.356 | 35.721 | 1.00 | 18.71 |
| ATOM | 16723 | OG1 | THR | 2502 | 26.565 | 8.386 | 34.685 | 1.00 | 18.62 |
| ATOM | 16724 | CG2 | THR | 2502 | 25.358 | 9.859 | 36.148 | 1.00 | 18.69 |
| ATOM | 16725 | C | THR | 2502 | 27.629 | 11.594 | 36.281 | 1.00 | 16.73 |
| ATOM | 16726 | O | THR | 2502 | 26.919 | 12.592 | 36.204 | 1.00 | 16.10 |
| ATOM | 16727 | N | VAL | 2503 | 28.441 | 11.355 | 37.301 | 1.00 | 16.73 |
| ATOM | 16728 | CA | VAL | 2503 | 28.582 | 12.280 | 38.416 | 1.00 | 19.12 |
| ATOM | 16729 | CB | VAL | 2503 | 29.336 | 11.588 | 39.565 | 1.00 | 21.79 |
| ATOM | 16730 | CG1 | VAL | 2503 | 29.985 | 12.604 | 40.454 | 1.00 | 26.41 |
| ATOM | 16731 | CG2 | VAL | 2503 | 28.365 | 10.712 | 40.355 | 1.00 | 18.83 |
| ATOM | 16732 | C | VAL | 2503 | 29.321 | 13.541 | 37.962 | 1.00 | 18.67 |
| ATOM | 16733 | O | VAL | 2503 | 29.040 | 14.649 | 38.435 | 1.00 | 18.94 |
| ATOM | 16734 | N | MET | 2504 | 30.260 | 13.362 | 37.037 | 1.00 | 18.34 |
| ATOM | 16735 | CA | MET | 2504 | 31.026 | 14.471 | 36.495 | 1.00 | 19.37 |
| ATOM | 16736 | CB | MET | 2504 | 32.211 | 13.958 | 35.669 | 1.00 | 22.55 |
| ATOM | 16737 | CG | MET | 2504 | 33.240 | 13.157 | 36.459 | 1.00 | 26.75 |
| ATOM | 16738 | SD | MET | 2504 | 34.195 | 14.200 | 37.555 | 1.00 | 33.58 |
| ATOM | 16739 | CE | MET | 2504 | 35.397 | 14.833 | 36.400 | 1.00 | 29.91 |
| ATOM | 16740 | C | MET | 2504 | 30.138 | 15.357 | 35.623 | 1.00 | 19.08 |
| ATOM | 16741 | O | MET | 2504 | 30.170 | 16.573 | 35.755 | 1.00 | 16.51 |
| ATOM | 16742 | N | ARG | 2505 | 29.341 | 14.757 | 34.738 | 1.00 | 18.93 |
| ATOM | 16743 | CA | ARG | 2505 | 28.476 | 15.553 | 33.883 | 1.00 | 19.02 |
| ATOM | 16744 | CB | ARG | 2505 | 27.748 | 14.672 | 32.853 | 1.00 | 19.49 |
| ATOM | 16745 | CG | ARG | 2505 | 28.673 | 13.917 | 31.888 | 1.00 | 22.49 |
| ATOM | 16746 | CD | ARG | 2505 | 27.903 | 13.299 | 30.717 | 1.00 | 23.75 |
| ATOM | 16747 | NE | ARG | 2505 | 28.708 | 12.314 | 29.985 | 1.00 | 25.72 |
| ATOM | 16748 | CZ | ARG | 2505 | 28.734 | 11.015 | 30.268 | 1.00 | 24.59 |
| ATOM | 16749 | NH1 | ARG | 2505 | 27.997 | 10.536 | 31.261 | 1.00 | 22.81 |
| ATOM | 16750 | NH2 | ARG | 2505 | 29.505 | 10.197 | 29.569 | 1.00 | 26.75 |
| ATOM | 16751 | C | ARG | 2505 | 27.451 | 16.298 | 34.728 | 1.00 | 17.69 |
| ATOM | 16752 | O | ARG | 2505 | 26.955 | 17.351 | 34.327 | 1.00 | 18.42 |
| ATOM | 16753 | N | ALA | 2506 | 27.145 | 15.755 | 35.902 | 1.00 | 17.09 |
| ATOM | 16754 | CA | ALA | 2506 | 26.164 | 16.363 | 36.800 | 1.00 | 18.48 |
| ATOM | 16755 | CB | ALA | 2506 | 25.652 | 15.325 | 37.797 | 1.00 | 17.12 |
| ATOM | 16756 | C | ALA | 2506 | 26.713 | 17.580 | 37.551 | 1.00 | 18.29 |
| ATOM | 16757 | O | ALA | 2506 | 25.957 | 18.298 | 38.208 | 1.00 | 18.33 |
| ATOM | 16758 | N | GLY | 2507 | 28.020 | 17.807 | 37.448 | 1.00 | 18.55 |
| ATOM | 16759 | CA | GLY | 2507 | 28.622 | 18.958 | 38.098 | 1.00 | 19.92 |
| ATOM | 16760 | C | GLY | 2507 | 29.863 | 18.732 | 38.943 | 1.00 | 20.81 |
| ATOM | 16761 | O | GLY | 2507 | 30.537 | 19.694 | 39.333 | 1.00 | 20.95 |
| ATOM | 16762 | N | ALA | 2508 | 30.183 | 17.478 | 39.238 | 1.00 | 20.50 |
| ATOM | 16763 | CA | ALA | 2508 | 31.354 | 17.193 | 40.062 | 1.00 | 19.08 |
| ATOM | 16764 | CB | ALA | 2508 | 31.328 | 15.748 | 40.526 | 1.00 | 18.45 |
| ATOM | 16765 | C | ALA | 2508 | 32.669 | 17.482 | 39.348 | 1.00 | 19.05 |
| ATOM | 16766 | O | ALA | 2508 | 32.776 | 17.332 | 38.134 | 1.00 | 18.04 |
| ATOM | 16767 | N | ASN | 2509 | 33.675 | 17.899 | 40.109 | 1.00 | 19.09 |
| ATOM | 16768 | CA | ASN | 2509 | 34.981 | 18.184 | 39.529 | 1.00 | 18.59 |
| ATOM | 16769 | CB | ASN | 2509 | 35.627 | 19.405 | 40.186 | 1.00 | 20.73 |
| ATOM | 16770 | CG | ASN | 2509 | 34.883 | 20.679 | 39.903 | 1.00 | 18.72 |
| ATOM | 16771 | OD1 | ASN | 2509 | 34.720 | 21.074 | 38.748 | 1.00 | 17.99 |
| ATOM | 16772 | ND2 | ASN | 2509 | 34.424 | 21.338 | 40.957 | 1.00 | 18.40 |
| ATOM | 16773 | C | ASN | 2509 | 35.905 | 17.000 | 39.756 | 1.00 | 19.28 |
| ATOM | 16774 | O | ASN | 2509 | 36.841 | 16.777 | 38.992 | 1.00 | 18.91 |
| ATOM | 16775 | N | MET | 2510 | 35.626 | 16.246 | 40.812 | 1.00 | 17.99 |
| ATOM | 16776 | CA | MET | 2510 | 36.448 | 15.115 | 41.203 | 1.00 | 19.45 |
| ATOM | 16777 | CB | MET | 2510 | 37.466 | 15.577 | 42.251 | 1.00 | 18.97 |
| ATOM | 16778 | CG | MET | 2510 | 38.433 | 14.524 | 42.759 | 1.00 | 23.50 |
| ATOM | 16779 | SD | MET | 2510 | 39.624 | 15.270 | 43.945 | 1.00 | 23.85 |
| ATOM | 16780 | CE | MET | 2510 | 41.036 | 15.618 | 42.863 | 1.00 | 24.62 |
| ATOM | 16781 | C | MET | 2510 | 35.576 | 14.020 | 41.784 | 1.00 | 20.19 |
| ATOM | 16782 | O | MET | 2510 | 34.505 | 14.289 | 42.327 | 1.00 | 18.96 |
| ATOM | 16783 | N | VAL | 2511 | 36.047 | 12.783 | 41.665 | 1.00 | 21.32 |
| ATOM | 16784 | CA | VAL | 2511 | 35.321 | 11.626 | 42.156 | 1.00 | 20.08 |
| ATOM | 16785 | CB | VAL | 2511 | 35.189 | 10.586 | 41.023 | 1.00 | 23.58 |
| ATOM | 16786 | CG1 | VAL | 2511 | 34.622 | 9.304 | 41.560 | 1.00 | 26.75 |
| ATOM | 16787 | CG2 | VAL | 2511 | 34.316 | 11.146 | 39.908 | 1.00 | 23.78 |
| ATOM | 16788 | C | VAL | 2511 | 36.035 | 10.991 | 43.347 | 1.00 | 19.91 |
| ATOM | 16789 | O | VAL | 2511 | 37.267 | 10.916 | 43.369 | 1.00 | 18.34 |
| ATOM | 16790 | N | LYS | 2512 | 35.268 | 10.547 | 44.340 | 1.00 | 17.54 |
| ATOM | 16791 | CA | LYS | 2512 | 35.850 | 9.902 | 45.513 | 1.00 | 18.90 |
| ATOM | 16792 | CB | LYS | 2512 | 35.473 | 10.648 | 46.803 | 1.00 | 19.77 |
| ATOM | 16793 | CG | LYS | 2512 | 36.064 | 9.979 | 48.044 | 1.00 | 21.40 |
| ATOM | 16794 | CD | LYS | 2512 | 36.198 | 10.915 | 49.233 | 1.00 | 23.09 |
| ATOM | 16795 | CE | LYS | 2512 | 34.908 | 11.031 | 50.024 | 1.00 | 23.73 |
| ATOM | 16796 | NZ | LYS | 2512 | 34.461 | 9.709 | 50.567 | 1.00 | 21.38 |
| ATOM | 16797 | C | LYS | 2512 | 35.407 | 8.442 | 45.620 | 1.00 | 19.54 |
| ATOM | 16798 | O | LYS | 2512 | 34.219 | 8.137 | 45.525 | 1.00 | 20.01 |
| ATOM | 16799 | N | ILE | 2513 | 36.370 | 7.545 | 45.817 | 1.00 | 17.93 |
| ATOM | 16800 | CA | ILE | 2513 | 36.085 | 6.117 | 45.923 | 1.00 | 17.40 |
| ATOM | 16801 | CB | ILE | 2513 | 36.447 | 5.384 | 44.607 | 1.00 | 17.97 |
| ATOM | 16802 | CG2 | ILE | 2513 | 35.518 | 5.836 | 43.489 | 1.00 | 17.02 |
| ATOM | 16803 | CG1 | ILE | 2513 | 37.896 | 5.694 | 44.213 | 1.00 | 18.73 |
| ATOM | 16804 | CD1 | ILE | 2513 | 38.353 | 4.974 | 42.946 | 1.00 | 18.72 |
| ATOM | 16805 | C | ILE | 2513 | 36.849 | 5.491 | 47.084 | 1.00 | 18.41 |
| ATOM | 16806 | O | ILE | 2513 | 37.994 | 5.858 | 47.353 | 1.00 | 18.45 |
| ATOM | 16807 | N | GLU | 2514 | 36.204 | 4.548 | 47.765 | 1.00 | 18.60 |
| ATOM | 16808 | CA | GLU | 2514 | 36.776 | 3.861 | 48.922 | 1.00 | 18.49 |
| ATOM | 16809 | CB | GLU | 2514 | 35.662 | 3.542 | 49.925 | 1.00 | 19.81 |
| ATOM | 16810 | CG | GLU | 2514 | 34.827 | 4.743 | 50.360 | 1.00 | 20.46 |
| ATOM | 16811 | CD | GLU | 2514 | 33.621 | 4.347 | 51.222 | 1.00 | 23.36 |
| ATOM | 16812 | OE1 | GLU | 2514 | 33.542 | 3.176 | 51.644 | 1.00 | 22.24 |
| ATOM | 16813 | OE2 | GLU | 2514 | 32.760 | 5.212 | 51.484 | 1.00 | 23.56 |
| ATOM | 16814 | C | GLU | 2514 | 37.512 | 2.566 | 48.571 | 1.00 | 19.66 |
| ATOM | 16815 | O | GLU | 2514 | 37.045 | 1.777 | 47.755 | 1.00 | 18.14 |
| ATOM | 16816 | N | GLY | 2515 | 38.665 | 2.339 | 49.189 | 1.00 | 20.64 |
| ATOM | 16817 | CA | GLY | 2515 | 39.389 | 1.117 | 48.896 | 1.00 | 20.29 |
| ATOM | 16818 | C | GLY | 2515 | 40.890 | 1.288 | 48.808 | 1.00 | 19.74 |
| ATOM | 16819 | O | GLY | 2515 | 41.393 | 2.371 | 48.501 | 1.00 | 18.88 |
| ATOM | 16820 | N | GLY | 2516 | 41.602 | 0.198 | 49.065 | 1.00 | 19.47 |
| ATOM | 16821 | CA | GLY | 2516 | 43.052 | 0.224 | 49.033 | 1.00 | 18.59 |
| ATOM | 16822 | C | GLY | 2516 | 43.687 | −0.132 | 47.703 | 1.00 | 18.48 |
| ATOM | 16823 | O | GLY | 2516 | 43.242 | 0.303 | 46.639 | 1.00 | 17.54 |
| ATOM | 16824 | N | GLU | 2517 | 44.725 | −0.955 | 47.769 | 1.00 | 20.76 |
| ATOM | 16825 | CA | GLU | 2517 | 45.467 | −1.341 | 46.580 | 1.00 | 23.29 |
| ATOM | 16826 | CB | GLU | 2517 | 46.637 | −2.242 | 46.974 | 1.00 | 27.23 |
| ATOM | 16827 | CG | GLU | 2517 | 47.689 | −2.380 | 45.888 | 1.00 | 32.03 |
| ATOM | 16828 | CD | GLU | 2517 | 48.937 | −3.090 | 46.367 | 1.00 | 36.09 |
| ATOM | 16829 | OE1 | GLU | 2517 | 49.853 | −3.300 | 45.539 | 1.00 | 38.84 |
| ATOM | 16830 | OE2 | GLU | 2517 | 49.005 | −3.435 | 47.569 | 1.00 | 37.39 |
| ATOM | 16831 | C | GLU | 2517 | 44.676 | −1.992 | 45.445 | 1.00 | 23.15 |
| ATOM | 16832 | O | GLU | 2517 | 45.079 | −1.892 | 44.286 | 1.00 | 22.58 |
| ATOM | 16833 | N | TRP | 2518 | 43.560 | −2.652 | 45.749 | 1.00 | 22.67 |
| ATOM | 16834 | CA | TRP | 2518 | 42.791 | −3.292 | 44.682 | 1.00 | 23.53 |
| ATOM | 16835 | CB | TRP | 2518 | 41.619 | −4.120 | 45.244 | 1.00 | 24.36 |
| ATOM | 16836 | CG | TRP | 2518 | 40.473 | −3.335 | 45.824 | 1.00 | 24.10 |
| ATOM | 16837 | CD2 | TRP | 2518 | 39.271 | −2.943 | 45.143 | 1.00 | 23.30 |
| ATOM | 16838 | CE2 | TRP | 2518 | 38.466 | −2.252 | 46.078 | 1.00 | 23.70 |
| ATOM | 16839 | CE3 | TRP | 2518 | 38.797 | −3.108 | 43.837 | 1.00 | 22.85 |
| ATOM | 16840 | CD1 | TRP | 2518 | 40.352 | −2.874 | 47.107 | 1.00 | 25.21 |
| ATOM | 16841 | NE1 | TRP | 2518 | 39.147 | −2.226 | 47.266 | 1.00 | 24.49 |
| ATOM | 16842 | CZ2 | TRP | 2518 | 37.209 | −1.726 | 45.745 | 1.00 | 22.66 |
| ATOM | 16843 | CZ3 | TRP | 2518 | 37.541 | −2.585 | 43.503 | 1.00 | 23.85 |
| ATOM | 16844 | CH2 | TRP | 2518 | 36.765 | −1.903 | 44.456 | 1.00 | 20.90 |
| ATOM | 16845 | C | TRP | 2518 | 42.267 | −2.282 | 43.666 | 1.00 | 22.18 |
| ATOM | 16846 | O | TRP | 2518 | 41.875 | −2.651 | 42.560 | 1.00 | 24.23 |
| ATOM | 16847 | N | LEU | 2519 | 42.283 | −1.006 | 44.042 | 1.00 | 21.97 |
| ATOM | 16848 | CA | LEU | 2519 | 41.804 | 0.080 | 43.184 | 1.00 | 19.59 |
| ATOM | 16849 | CB | LEU | 2519 | 41.185 | 1.180 | 44.043 | 1.00 | 20.80 |
| ATOM | 16850 | CG | LEU | 2519 | 39.771 | 0.921 | 44.553 | 1.00 | 20.95 |
| ATOM | 16851 | CD1 | LEU | 2519 | 39.347 | 2.037 | 45.517 | 1.00 | 20.03 |
| ATOM | 16852 | CD2 | LEU | 2519 | 38.824 | 0.853 | 43.358 | 1.00 | 19.11 |
| ATOM | 16853 | C | LEU | 2519 | 42.855 | 0.724 | 42.292 | 1.00 | 20.90 |
| ATOM | 16854 | O | LEU | 2519 | 42.525 | 1.556 | 41.444 | 1.00 | 20.68 |
| ATOM | 16855 | N | VAL | 2520 | 44.117 | 0.361 | 42.483 | 1.00 | 19.40 |
| ATOM | 16856 | CA | VAL | 2520 | 45.186 | 0.955 | 41.692 | 1.00 | 18.98 |
| ATOM | 16857 | CB | VAL | 2520 | 46.513 | 0.172 | 41.864 | 1.00 | 19.23 |
| ATOM | 16858 | CG1 | VAL | 2520 | 47.524 | 0.629 | 40.825 | 1.00 | 20.17 |
| ATOM | 16859 | CG2 | VAL | 2520 | 47.073 | 0.413 | 43.262 | 1.00 | 21.65 |
| ATOM | 16860 | C | VAL | 2520 | 44.870 | 1.070 | 40.206 | 1.00 | 18.55 |
| ATOM | 16861 | O | VAL | 2520 | 44.981 | 2.149 | 39.630 | 1.00 | 18.33 |
| ATOM | 16862 | N | GLU | 2521 | 44.475 | −0.037 | 39.590 | 1.00 | 17.61 |
| ATOM | 16863 | CA | GLU | 2521 | 44.159 | −0.037 | 38.168 | 1.00 | 21.27 |
| ATOM | 16864 | CB | GLU | 2521 | 43.770 | −1.448 | 37.729 | 1.00 | 25.74 |
| ATOM | 16865 | CG | GLU | 2521 | 43.404 | −1.568 | 36.261 | 1.00 | 31.82 |
| ATOM | 16866 | CD | GLU | 2521 | 43.139 | −3.009 | 35.856 | 1.00 | 35.80 |
| ATOM | 16867 | OE1 | GLU | 2521 | 42.203 | −3.633 | 36.408 | 1.00 | 37.34 |
| ATOM | 16868 | OE2 | GLU | 2521 | 43.871 | −3.520 | 34.985 | 1.00 | 39.34 |
| ATOM | 16869 | C | GLU | 2521 | 43.037 | 0.942 | 37.828 | 1.00 | 19.73 |
| ATOM | 16870 | O | GLU | 2521 | 43.137 | 1.721 | 36.879 | 1.00 | 18.47 |
| ATOM | 16871 | N | THR | 2522 | 41.967 | 0.909 | 38.611 | 1.00 | 18.72 |
| ATOM | 16872 | CA | THR | 2522 | 40.834 | 1.789 | 38.360 | 1.00 | 17.06 |
| ATOM | 16873 | CB | THR | 2522 | 39.696 | 1.466 | 39.341 | 1.00 | 17.53 |
| ATOM | 16874 | OG1 | THR | 2522 | 39.327 | 0.087 | 39.182 | 1.00 | 18.32 |
| ATOM | 16875 | CG2 | THR | 2522 | 38.481 | 2.339 | 39.074 | 1.00 | 16.11 |
| ATOM | 16876 | C | THR | 2522 | 41.232 | 3.256 | 38.469 | 1.00 | 17.09 |
| ATOM | 16877 | O | THR | 2522 | 40.808 | 4.089 | 37.668 | 1.00 | 16.74 |
| ATOM | 16878 | N | VAL | 2523 | 42.060 | 3.580 | 39.452 | 1.00 | 18.90 |
| ATOM | 16879 | CA | VAL | 2523 | 42.488 | 4.968 | 39.614 | 1.00 | 18.66 |
| ATOM | 16880 | CB | VAL | 2523 | 43.295 | 5.155 | 40.910 | 1.00 | 19.35 |
| ATOM | 16881 | CG1 | VAL | 2523 | 43.779 | 6.595 | 41.011 | 1.00 | 20.95 |
| ATOM | 16882 | CG2 | VAL | 2523 | 42.442 | 4.799 | 42.116 | 1.00 | 18.48 |
| ATOM | 16883 | C | VAL | 2523 | 43.345 | 5.398 | 38.419 | 1.00 | 18.29 |
| ATOM | 16884 | O | VAL | 2523 | 43.141 | 6.476 | 37.858 | 1.00 | 18.36 |
| ATOM | 16885 | N | GLN | 2524 | 44.309 | 4.563 | 38.038 | 1.00 | 16.98 |
| ATOM | 16886 | CA | GLN | 2524 | 45.174 | 4.894 | 36.907 | 1.00 | 19.97 |
| ATOM | 16887 | CB | GLN | 2524 | 46.169 | 3.761 | 36.626 | 1.00 | 21.97 |
| ATOM | 16888 | CG | GLN | 2524 | 47.125 | 3.462 | 37.769 | 1.00 | 28.49 |
| ATOM | 16889 | CD | GLN | 2524 | 48.108 | 2.355 | 37.429 | 1.00 | 32.31 |
| ATOM | 16890 | OE1 | GLN | 2524 | 47.711 | 1.273 | 36.998 | 1.00 | 33.90 |
| ATOM | 16891 | NE2 | GLN | 2524 | 49.400 | 2.619 | 37.627 | 1.00 | 32.72 |
| ATOM | 16892 | C | GLN | 2524 | 44.359 | 5.153 | 35.648 | 1.00 | 20.47 |
| ATOM | 16893 | O | GLN | 2524 | 44.572 | 6.146 | 34.950 | 1.00 | 19.34 |
| ATOM | 16894 | N | MET | 2525 | 43.420 | 4.258 | 35.360 | 1.00 | 19.30 |
| ATOM | 16895 | CA | MET | 2525 | 42.600 | 4.385 | 34.163 | 1.00 | 20.94 |
| ATOM | 16896 | CB | MET | 2525 | 41.853 | 3.076 | 33.908 | 1.00 | 20.76 |
| ATOM | 16897 | CG | MET | 2525 | 42.796 | 1.931 | 33.555 | 1.00 | 22.97 |
| ATOM | 16898 | SD | MET | 2525 | 41.953 | 0.379 | 33.211 | 1.00 | 25.66 |
| ATOM | 16899 | CE | MET | 2525 | 41.613 | 0.585 | 31.487 | 1.00 | 26.72 |
| ATOM | 16900 | C | MET | 2525 | 41.638 | 5.558 | 34.213 | 1.00 | 21.37 |
| ATOM | 16901 | O | MET | 2525 | 41.439 | 6.233 | 33.208 | 1.00 | 21.66 |
| ATOM | 16902 | N | LEU | 2526 | 41.044 | 5.808 | 35.375 | 1.00 | 21.24 |
| ATOM | 16903 | CA | LEU | 2526 | 40.129 | 6.935 | 35.503 | 1.00 | 20.80 |
| ATOM | 16904 | CB | LEU | 2526 | 39.502 | 6.964 | 36.900 | 1.00 | 17.52 |
| ATOM | 16905 | CG | LEU | 2526 | 38.292 | 6.075 | 37.149 | 1.00 | 16.74 |
| ATOM | 16906 | CD1 | LEU | 2526 | 37.996 | 6.041 | 38.641 | 1.00 | 15.92 |
| ATOM | 16907 | CD2 | LEU | 2526 | 37.095 | 6.606 | 36.373 | 1.00 | 16.60 |
| ATOM | 16908 | C | LEU | 2526 | 40.878 | 8.241 | 35.263 | 1.00 | 21.12 |
| ATOM | 16909 | O | LEU | 2526 | 40.404 | 9.124 | 34.546 | 1.00 | 21.66 |
| ATOM | 16910 | N | THR | 2527 | 42.059 | 8.353 | 35.860 | 1.00 | 22.05 |
| ATOM | 16911 | CA | THR | 2527 | 42.875 | 9.555 | 35.725 | 1.00 | 24.14 |
| ATOM | 16912 | CB | THR | 2527 | 44.186 | 9.419 | 36.521 | 1.00 | 24.39 |
| ATOM | 16913 | OG1 | THR | 2527 | 43.879 | 9.270 | 37.914 | 1.00 | 27.58 |
| ATOM | 16914 | CG2 | THR | 2527 | 45.061 | 10.656 | 36.334 | 1.00 | 27.09 |
| ATOM | 16915 | C | THR | 2527 | 43.217 | 9.883 | 34.276 | 1.00 | 25.83 |
| ATOM | 16916 | O | THR | 2527 | 43.061 | 11.020 | 33.838 | 1.00 | 26.77 |
| ATOM | 16917 | N | GLU | 2528 | 43.679 | 8.888 | 33.529 | 1.00 | 27.93 |
| ATOM | 16918 | CA | GLU | 2528 | 44.042 | 9.127 | 32.138 | 1.00 | 29.80 |
| ATOM | 16919 | CB | GLU | 2528 | 44.815 | 7.935 | 31.578 | 1.00 | 31.80 |
| ATOM | 16920 | CG | GLU | 2528 | 43.995 | 6.697 | 31.350 | 1.00 | 32.54 |
| ATOM | 16921 | CD | GLU | 2528 | 44.836 | 5.567 | 30.791 | 1.00 | 34.95 |
| ATOM | 16922 | OE1 | GLU | 2528 | 45.737 | 5.857 | 29.977 | 1.00 | 35.69 |
| ATOM | 16923 | OE2 | GLU | 2528 | 44.599 | 4.397 | 31.157 | 1.00 | 32.66 |
| ATOM | 16924 | C | GLU | 2528 | 42.827 | 9.429 | 31.265 | 1.00 | 28.28 |
| ATOM | 16925 | O | GLU | 2528 | 42.971 | 9.896 | 30.135 | 1.00 | 28.14 |
| ATOM | 16926 | N | ARG | 2529 | 41.634 | 9.174 | 31.797 | 1.00 | 28.80 |
| ATOM | 16927 | CA | ARG | 2529 | 40.395 | 9.431 | 31.069 | 1.00 | 28.26 |
| ATOM | 16928 | CB | ARG | 2529 | 39.452 | 8.230 | 31.205 | 1.00 | 26.83 |
| ATOM | 16929 | CG | ARG | 2529 | 39.946 | 7.014 | 30.427 | 1.00 | 26.61 |
| ATOM | 16930 | CD | ARG | 2529 | 39.231 | 5.732 | 30.796 | 1.00 | 23.98 |
| ATOM | 16931 | NE | ARG | 2529 | 39.737 | 4.615 | 30.003 | 1.00 | 24.06 |
| ATOM | 16932 | CZ | ARG | 2529 | 40.992 | 4.180 | 30.041 | 1.00 | 25.03 |
| ATOM | 16933 | NH1 | ARG | 2529 | 41.879 | 4.759 | 30.838 | 1.00 | 23.39 |
| ATOM | 16934 | NH2 | ARG | 2529 | 41.368 | 3.171 | 29.270 | 1.00 | 24.77 |
| ATOM | 16935 | C | ARG | 2529 | 39.702 | 10.713 | 31.530 | 1.00 | 28.54 |
| ATOM | 16936 | O | ARG | 2529 | 38.478 | 10.827 | 31.465 | 1.00 | 28.70 |
| ATOM | 16937 | N | ALA | 2530 | 40.503 | 11.667 | 32.001 | 1.00 | 28.59 |
| ATOM | 16938 | CA | ALA | 2530 | 40.018 | 12.970 | 32.452 | 1.00 | 26.75 |
| ATOM | 16939 | CB | ALA | 2530 | 39.218 | 13.637 | 31.332 | 1.00 | 29.24 |
| ATOM | 16940 | C | ALA | 2530 | 39.196 | 12.970 | 33.737 | 1.00 | 26.34 |
| ATOM | 16941 | O | ALA | 2530 | 38.391 | 13.876 | 33.953 | 1.00 | 25.02 |
| ATOM | 16942 | N | VAL | 2531 | 39.400 | 11.974 | 34.595 | 1.00 | 23.38 |
| ATOM | 16943 | CA | VAL | 2531 | 38.655 | 11.907 | 35.849 | 1.00 | 23.24 |
| ATOM | 16944 | CB | VAL | 2531 | 37.802 | 10.617 | 35.933 | 1.00 | 22.38 |
| ATOM | 16945 | CG1 | VAL | 2531 | 37.001 | 10.605 | 37.222 | 1.00 | 24.98 |
| ATOM | 16946 | CG2 | VAL | 2531 | 36.874 | 10.530 | 34.740 | 1.00 | 23.60 |
| ATOM | 16947 | C | VAL | 2531 | 39.549 | 11.968 | 37.089 | 1.00 | 22.08 |
| ATOM | 16948 | O | VAL | 2531 | 40.180 | 10.979 | 37.464 | 1.00 | 20.71 |
| ATOM | 16949 | N | PRO | 2532 | 39.618 | 13.137 | 37.743 | 1.00 | 22.24 |
| ATOM | 16950 | CD | PRO | 2532 | 38.951 | 14.412 | 37.439 | 1.00 | 23.52 |
| ATOM | 16951 | CA | PRO | 2532 | 40.450 | 13.259 | 38.944 | 1.00 | 22.45 |
| ATOM | 16952 | CB | PRO | 2532 | 40.414 | 14.757 | 39.238 | 1.00 | 22.66 |
| ATOM | 16953 | CG | PRO | 2532 | 39.070 | 15.148 | 38.756 | 1.00 | 25.65 |
| ATOM | 16954 | C | PRO | 2532 | 39.836 | 12.421 | 40.057 | 1.00 | 21.12 |
| ATOM | 16955 | O | PRO | 2532 | 38.623 | 12.446 | 40.255 | 1.00 | 21.74 |
| ATOM | 16956 | N | VAL | 2533 | 40.673 | 11.678 | 40.773 | 1.00 | 19.42 |
| ATOM | 16957 | CA | VAL | 2533 | 40.208 | 10.802 | 41.837 | 1.00 | 18.80 |
| ATOM | 16958 | CB | VAL | 2533 | 40.670 | 9.345 | 41.596 | 1.00 | 18.15 |
| ATOM | 16959 | CG1 | VAL | 2533 | 40.209 | 8.462 | 42.736 | 1.00 | 19.03 |
| ATOM | 16960 | CG2 | VAL | 2533 | 40.134 | 8.832 | 40.263 | 1.00 | 18.13 |
| ATOM | 16961 | C | VAL | 2533 | 40.696 | 11.181 | 43.220 | 1.00 | 18.90 |
| ATOM | 16962 | O | VAL | 2533 | 41.844 | 11.587 | 43.389 | 1.00 | 19.13 |
| ATOM | 16963 | N | CYS | 2534 | 39.813 | 11.044 | 44.204 | 1.00 | 17.65 |
| ATOM | 16964 | CA | CYS | 2534 | 40.143 | 11.296 | 45.608 | 1.00 | 17.23 |
| ATOM | 16965 | CB | CYS | 2534 | 39.125 | 12.250 | 46.259 | 1.00 | 16.92 |
| ATOM | 16966 | SG | CYS | 2534 | 39.435 | 12.575 | 48.025 | 1.00 | 17.39 |
| ATOM | 16967 | C | CYS | 2534 | 40.038 | 9.922 | 46.265 | 1.00 | 17.85 |
| ATOM | 16968 | O | CYS | 2534 | 39.004 | 9.258 | 46.159 | 1.00 | 20.42 |
| ATOM | 16969 | N | GLY | 2535 | 41.107 | 9.481 | 46.917 | 1.00 | 16.91 |
| ATOM | 16970 | CA | GLY | 2535 | 41.084 | 8.192 | 47.580 | 1.00 | 17.14 |
| ATOM | 16971 | C | GLY | 2535 | 40.382 | 8.290 | 48.921 | 1.00 | 18.33 |
| ATOM | 16972 | O | GLY | 2535 | 40.041 | 9.388 | 49.362 | 1.00 | 19.66 |
| ATOM | 16973 | N | HIS | 2536 | 40.168 | 7.149 | 49.568 | 1.00 | 17.24 |
| ATOM | 16974 | CA | HIS | 2536 | 39.504 | 7.106 | 50.869 | 1.00 | 18.82 |
| ATOM | 16975 | CB | HIS | 2536 | 37.980 | 7.162 | 50.683 | 1.00 | 20.25 |
| ATOM | 16976 | CG | HIS | 2536 | 37.207 | 7.331 | 51.956 | 1.00 | 18.55 |
| ATOM | 16977 | CD2 | HIS | 2536 | 37.537 | 7.091 | 53.248 | 1.00 | 19.79 |
| ATOM | 16978 | ND1 | HIS | 2536 | 35.897 | 7.761 | 51.971 | 1.00 | 20.89 |
| ATOM | 16979 | CE1 | HIS | 2536 | 35.453 | 7.777 | 53.215 | 1.00 | 19.89 |
| ATOM | 16980 | NE2 | HIS | 2536 | 36.428 | 7.373 | 54.010 | 1.00 | 20.24 |
| ATOM | 16981 | C | HIS | 2536 | 39.922 | 5.823 | 51.575 | 1.00 | 18.66 |
| ATOM | 16982 | O | HIS | 2536 | 39.620 | 4.717 | 51.123 | 1.00 | 19.56 |
| ATOM | 16983 | N | LEU | 2537 | 40.639 | 5.984 | 52.680 | 1.00 | 18.70 |
| ATOM | 16984 | CA | LEU | 2537 | 41.127 | 4.855 | 53.455 | 1.00 | 17.87 |
| ATOM | 16985 | CB | LEU | 2537 | 42.650 | 4.771 | 53.338 | 1.00 | 19.01 |
| ATOM | 16986 | CG | LEU | 2537 | 43.229 | 4.387 | 51.973 | 1.00 | 16.20 |
| ATOM | 16987 | CD1 | LEU | 2537 | 44.757 | 4.537 | 51.995 | 1.00 | 16.71 |
| ATOM | 16988 | CD2 | LEU | 2537 | 42.851 | 2.929 | 51.653 | 1.00 | 17.55 |
| ATOM | 16989 | C | LEU | 2537 | 40.737 | 4.977 | 54.926 | 1.00 | 19.09 |
| ATOM | 16990 | O | LEU | 2537 | 40.273 | 6.026 | 55.371 | 1.00 | 20.25 |
| ATOM | 16991 | N | GLY | 2538 | 40.960 | 3.901 | 55.670 | 1.00 | 18.13 |
| ATOM | 16992 | CA | GLY | 2538 | 40.633 | 3.882 | 57.078 | 1.00 | 19.82 |
| ATOM | 16993 | C | GLY | 2538 | 39.275 | 3.253 | 57.287 | 1.00 | 21.82 |
| ATOM | 16994 | O | GLY | 2538 | 39.013 | 2.131 | 56.843 | 1.00 | 22.22 |
| ATOM | 16995 | N | LEU | 2539 | 38.394 | 3.980 | 57.959 | 1.00 | 21.58 |
| ATOM | 16996 | CA | LEU | 2539 | 37.059 | 3.470 | 58.193 | 1.00 | 22.62 |
| ATOM | 16997 | CB | LEU | 2539 | 36.478 | 4.096 | 59.464 | 1.00 | 23.08 |
| ATOM | 16998 | CG | LEU | 2539 | 35.394 | 3.309 | 60.206 | 1.00 | 25.71 |
| ATOM | 16999 | CD1 | LEU | 2539 | 34.896 | 4.150 | 61.377 | 1.00 | 27.15 |
| ATOM | 17000 | CD2 | LEU | 2539 | 34.254 | 2.962 | 59.280 | 1.00 | 24.37 |
| ATOM | 17001 | C | LEU | 2539 | 36.213 | 3.830 | 56.977 | 1.00 | 22.01 |
| ATOM | 17002 | O | LEU | 2539 | 35.665 | 4.931 | 56.897 | 1.00 | 24.68 |
| ATOM | 17003 | N | THR | 2540 | 36.139 | 2.909 | 56.020 | 1.00 | 21.79 |
| ATOM | 17004 | CA | THR | 2540 | 35.350 | 3.100 | 54.801 | 1.00 | 23.60 |
| ATOM | 17005 | CB | THR | 2540 | 35.991 | 2.337 | 53.599 | 1.00 | 22.72 |
| ATOM | 17006 | OG1 | THR | 2540 | 36.312 | 0.996 | 53.991 | 1.00 | 22.38 |
| ATOM | 17007 | CG2 | THR | 2540 | 37.258 | 3.035 | 53.143 | 1.00 | 25.28 |
| ATOM | 17008 | C | THR | 2540 | 33.934 | 2.574 | 55.072 | 1.00 | 23.37 |
| ATOM | 17009 | O | THR | 2540 | 33.680 | 1.371 | 54.992 | 1.00 | 23.82 |
| ATOM | 17010 | N | PRO | 2541 | 32.997 | 3.483 | 55.403 | 1.00 | 24.67 |
| ATOM | 17011 | CD | PRO | 2541 | 33.189 | 4.941 | 55.330 | 1.00 | 25.29 |
| ATOM | 17012 | CA | PRO | 2541 | 31.595 | 3.165 | 55.711 | 1.00 | 23.91 |
| ATOM | 17013 | CB | PRO | 2541 | 30.927 | 4.543 | 55.801 | 1.00 | 25.13 |
| ATOM | 17014 | CG | PRO | 2541 | 31.817 | 5.420 | 54.989 | 1.00 | 29.14 |
| ATOM | 17015 | C | PRO | 2541 | 30.866 | 2.212 | 54.777 | 1.00 | 22.03 |
| ATOM | 17016 | O | PRO | 2541 | 29.964 | 1.489 | 55.212 | 1.00 | 21.27 |
| ATOM | 17017 | N | GLN | 2542 | 31.241 | 2.196 | 53.504 | 1.00 | 21.17 |
| ATOM | 17018 | CA | GLN | 2542 | 30.593 | 1.280 | 52.562 | 1.00 | 20.34 |
| ATOM | 17019 | CB | GLN | 2542 | 31.081 | 1.542 | 51.126 | 1.00 | 19.40 |
| ATOM | 17020 | CG | GLN | 2542 | 30.468 | 2.776 | 50.462 | 1.00 | 19.10 |
| ATOM | 17021 | CD | GLN | 2542 | 31.057 | 3.072 | 49.090 | 1.00 | 20.00 |
| ATOM | 17022 | OE1 | GLN | 2542 | 31.534 | 2.172 | 48.399 | 1.00 | 21.78 |
| ATOM | 17023 | NE2 | GLN | 2542 | 31.014 | 4.335 | 48.686 | 1.00 | 18.53 |
| ATOM | 17024 | C | GLN | 2542 | 30.887 | −0.166 | 52.962 | 1.00 | 20.22 |
| ATOM | 17025 | O | GLN | 2542 | 30.106 | −1.067 | 52.659 | 1.00 | 21.15 |
| ATOM | 17026 | N | SER | 2543 | 32.005 | −0.381 | 53.655 | 1.00 | 21.08 |
| ATOM | 17027 | CA | SER | 2543 | 32.392 | −1.725 | 54.089 | 1.00 | 20.09 |
| ATOM | 17028 | CB | SER | 2543 | 33.903 | −1.934 | 53.925 | 1.00 | 22.29 |
| ATOM | 17029 | OG | SER | 2543 | 34.275 | −2.037 | 52.553 | 1.00 | 22.70 |
| ATOM | 17030 | C | SER | 2543 | 32.009 | −2.008 | 55.541 | 1.00 | 20.40 |
| ATOM | 17031 | O | SER | 2543 | 32.533 | −2.938 | 56.162 | 1.00 | 19.84 |
| ATOM | 17032 | N | VAL | 2544 | 31.084 | −1.218 | 56.077 | 1.00 | 19.14 |
| ATOM | 17033 | CA | VAL | 2544 | 30.644 | −1.391 | 57.455 | 1.00 | 20.23 |
| ATOM | 17034 | CB | VAL | 2544 | 29.416 | −0.473 | 57.774 | 1.00 | 21.04 |
| ATOM | 17035 | CG1 | VAL | 2544 | 28.242 | −0.812 | 56.867 | 1.00 | 22.60 |
| ATOM | 17036 | CG2 | VAL | 2544 | 29.031 | −0.615 | 59.235 | 1.00 | 24.52 |
| ATOM | 17037 | C | VAL | 2544 | 30.301 | −2.847 | 57.775 | 1.00 | 19.12 |
| ATOM | 17038 | O | VAL | 2544 | 30.623 | −3.343 | 58.854 | 1.00 | 17.37 |
| ATOM | 17039 | N | ASN | 2545 | 29.659 | −3.533 | 56.835 | 1.00 | 20.26 |
| ATOM | 17040 | CA | ASN | 2545 | 29.288 | −4.923 | 57.041 | 1.00 | 21.25 |
| ATOM | 17041 | CB | ASN | 2545 | 28.350 | −5.383 | 55.921 | 1.00 | 20.97 |
| ATOM | 17042 | CG | ASN | 2545 | 27.009 | −4.683 | 55.969 | 1.00 | 19.71 |
| ATOM | 17043 | OD1 | ASN | 2545 | 26.215 | −4.912 | 56.881 | 1.00 | 19.36 |
| ATOM | 17044 | ND2 | ASN | 2545 | 26.755 | −3.806 | 54.997 | 1.00 | 20.75 |
| ATOM | 17045 | C | ASN | 2545 | 30.521 | −5.820 | 57.101 | 1.00 | 22.83 |
| ATOM | 17046 | O | ASN | 2545 | 30.499 | −6.877 | 57.726 | 1.00 | 23.72 |
| ATOM | 17047 | N | ILE | 2546 | 31.594 | −5.395 | 56.443 | 1.00 | 22.99 |
| ATOM | 17048 | CA | ILE | 2546 | 32.839 | −6.154 | 56.447 | 1.00 | 24.98 |
| ATOM | 17049 | CB | ILE | 2546 | 33.842 | −5.623 | 55.379 | 1.00 | 25.25 |
| ATOM | 17050 | CG2 | ILE | 2546 | 35.189 | −6.332 | 55.529 | 1.00 | 24.01 |
| ATOM | 17051 | CG1 | ILE | 2546 | 33.295 | −5.852 | 53.968 | 1.00 | 25.29 |
| ATOM | 17052 | CD1 | ILE | 2546 | 33.166 | −7.330 | 53.587 | 1.00 | 25.88 |
| ATOM | 17053 | C | ILE | 2546 | 33.485 | −6.028 | 57.827 | 1.00 | 26.31 |
| ATOM | 17054 | O | ILE | 2546 | 33.852 | −7.027 | 58.445 | 1.00 | 25.76 |
| ATOM | 17055 | N | PHE | 2547 | 33.606 | −4.794 | 58.309 | 1.00 | 26.71 |
| ATOM | 17056 | CA | PHE | 2547 | 34.214 | −4.534 | 59.615 | 1.00 | 29.32 |
| ATOM | 17057 | CB | PHE | 2547 | 34.482 | −3.033 | 59.802 | 1.00 | 29.87 |
| ATOM | 17058 | CG | PHE | 2547 | 35.258 | −2.401 | 58.679 | 1.00 | 32.32 |
| ATOM | 17059 | CD1 | PHE | 2547 | 36.474 | −2.939 | 58.258 | 1.00 | 33.03 |
| ATOM | 17060 | CD2 | PHE | 2547 | 34.782 | −1.253 | 58.054 | 1.00 | 32.76 |
| ATOM | 17061 | CE1 | PHE | 2547 | 37.204 | −2.342 | 57.229 | 1.00 | 32.42 |
| ATOM | 17062 | CE2 | PHE | 2547 | 35.504 | −0.647 | 57.024 | 1.00 | 33.54 |
| ATOM | 17063 | CZ | PHE | 2547 | 36.719 | −1.195 | 56.612 | 1.00 | 33.74 |
| ATOM | 17064 | C | PHE | 2547 | 33.325 | −5.011 | 60.756 | 1.00 | 30.19 |
| ATOM | 17065 | O | PHE | 2547 | 33.814 | −5.329 | 61.837 | 1.00 | 31.92 |
| ATOM | 17066 | N | GLY | 2548 | 32.020 | −5.063 | 60.509 | 1.00 | 30.16 |
| ATOM | 17067 | CA | GLY | 2548 | 31.093 | −5.483 | 61.541 | 1.00 | 30.32 |
| ATOM | 17068 | C | GLY | 2548 | 30.665 | −4.284 | 62.367 | 1.00 | 31.76 |
| ATOM | 17069 | O | GLY | 2548 | 30.281 | −4.412 | 63.529 | 1.00 | 33.19 |
| ATOM | 17070 | N | GLY | 2549 | 30.741 | −3.106 | 61.756 | 1.00 | 32.09 |
| ATOM | 17071 | CA | GLY | 2549 | 30.361 | −1.882 | 62.438 | 1.00 | 31.70 |
| ATOM | 17072 | C | GLY | 2549 | 31.326 | −0.764 | 62.095 | 1.00 | 31.81 |
| ATOM | 17073 | O | GLY | 2549 | 32.223 | −0.943 | 61.273 | 1.00 | 30.10 |
| ATOM | 17074 | N | TYR | 2550 | 31.151 | 0.394 | 62.722 | 1.00 | 32.22 |
| ATOM | 17075 | CA | TYR | 2550 | 32.030 | 1.525 | 62.467 | 1.00 | 33.59 |
| ATOM | 17076 | CB | TYR | 2550 | 31.229 | 2.827 | 62.456 | 1.00 | 34.84 |
| ATOM | 17077 | CG | TYR | 2550 | 30.084 | 2.806 | 61.470 | 1.00 | 36.99 |
| ATOM | 17078 | CD1 | TYR | 2550 | 28.863 | 2.207 | 61.800 | 1.00 | 37.25 |
| ATOM | 17079 | CE1 | TYR | 2550 | 27.821 | 2.140 | 60.875 | 1.00 | 38.43 |
| ATOM | 17080 | CD2 | TYR | 2550 | 30.233 | 3.339 | 60.189 | 1.00 | 37.03 |
| ATOM | 17081 | CE2 | TYR | 2550 | 29.201 | 3.275 | 59.255 | 1.00 | 37.75 |
| ATOM | 17082 | CZ | TYR | 2550 | 27.999 | 2.675 | 59.600 | 1.00 | 38.27 |
| ATOM | 17083 | OH | TYR | 2550 | 26.986 | 2.600 | 58.671 | 1.00 | 36.43 |
| ATOM | 17084 | C | TYR | 2550 | 33.105 | 1.564 | 63.546 | 1.00 | 34.34 |
| ATOM | 17085 | O | TYR | 2550 | 32.948 | 2.226 | 64.572 | 1.00 | 35.56 |
| ATOM | 17086 | N | LYS | 2551 | 34.194 | 0.840 | 63.306 | 1.00 | 32.76 |
| ATOM | 17087 | CA | LYS | 2551 | 35.298 | 0.752 | 64.254 | 1.00 | 32.19 |
| ATOM | 17088 | CB | LYS | 2551 | 35.598 | −0.719 | 64.546 | 1.00 | 34.81 |
| ATOM | 17089 | CG | LYS | 2551 | 34.364 | −1.541 | 64.900 | 1.00 | 38.01 |
| ATOM | 17090 | CD | LYS | 2551 | 34.699 | −3.015 | 65.008 | 1.00 | 40.22 |
| ATOM | 17091 | CE | LYS | 2551 | 33.462 | −3.851 | 65.283 | 1.00 | 41.88 |
| ATOM | 17092 | NZ | LYS | 2551 | 33.802 | −5.302 | 65.396 | 1.00 | 44.79 |
| ATOM | 17093 | C | LYS | 2551 | 36.553 | 1.435 | 63.719 | 1.00 | 30.37 |
| ATOM | 17094 | O | LYS | 2551 | 36.786 | 1.462 | 62.513 | 1.00 | 30.25 |
| ATOM | 17095 | N | VAL | 2552 | 37.363 | 1.982 | 64.619 | 1.00 | 29.43 |
| ATOM | 17096 | CA | VAL | 2552 | 38.590 | 2.658 | 64.215 | 1.00 | 28.03 |
| ATOM | 17097 | CB | VAL | 2552 | 39.312 | 3.274 | 65.433 | 1.00 | 28.51 |
| ATOM | 17098 | CG1 | VAL | 2552 | 40.619 | 3.922 | 65.001 | 1.00 | 26.74 |
| ATOM | 17099 | CG2 | VAL | 2552 | 38.410 | 4.304 | 66.096 | 1.00 | 26.18 |
| ATOM | 17100 | C | VAL | 2552 | 39.512 | 1.662 | 63.518 | 1.00 | 27.85 |
| ATOM | 17101 | O | VAL | 2552 | 39.697 | 0.538 | 63.988 | 1.00 | 26.93 |
| ATOM | 17102 | N | GLN | 2553 | 40.085 | 2.080 | 62.395 | 1.00 | 27.87 |
| ATOM | 17103 | CA | GLN | 2553 | 40.973 | 1.217 | 61.626 | 1.00 | 29.57 |
| ATOM | 17104 | CB | GLN | 2553 | 40.523 | 1.196 | 60.162 | 1.00 | 30.19 |
| ATOM | 17105 | CG | GLN | 2553 | 40.399 | −0.196 | 59.554 | 1.00 | 34.54 |
| ATOM | 17106 | CD | GLN | 2553 | 39.402 | −1.069 | 60.292 | 1.00 | 34.33 |
| ATOM | 17107 | OE1 | GLN | 2553 | 38.286 | −0.641 | 60.594 | 1.00 | 35.02 |
| ATOM | 17108 | NE2 | GLN | 2553 | 39.798 | −2.303 | 60.582 | 1.00 | 37.14 |
| ATOM | 17109 | C | GLN | 2553 | 42.419 | 1.695 | 61.715 | 1.00 | 29.23 |
| ATOM | 17110 | O | GLN | 2553 | 42.686 | 2.813 | 62.152 | 1.00 | 29.38 |
| ATOM | 17111 | N | GLY | 2554 | 43.346 | 0.837 | 61.303 | 1.00 | 30.12 |
| ATOM | 17112 | CA | GLY | 2554 | 44.752 | 1.200 | 61.328 | 1.00 | 31.55 |
| ATOM | 17113 | C | GLY | 2554 | 45.499 | 0.751 | 62.568 | 1.00 | 33.25 |
| ATOM | 17114 | O | GLY | 2554 | 46.730 | 0.754 | 62.590 | 1.00 | 33.26 |
| ATOM | 17115 | N | ARG | 2555 | 44.755 | 0.371 | 63.602 | 1.00 | 33.75 |
| ATOM | 17116 | CA | ARG | 2555 | 45.350 | −0.088 | 64.851 | 1.00 | 34.75 |
| ATOM | 17117 | CB | ARG | 2555 | 44.253 | −0.548 | 65.818 | 1.00 | 36.93 |
| ATOM | 17118 | CG | ARG | 2555 | 43.309 | 0.558 | 66.303 | 1.00 | 39.18 |
| ATOM | 17119 | CD | ARG | 2555 | 43.894 | 1.309 | 67.489 | 1.00 | 40.08 |
| ATOM | 17120 | NE | ARG | 2555 | 42.993 | 2.331 | 68.030 | 1.00 | 39.43 |
| ATOM | 17121 | CZ | ARG | 2555 | 41.807 | 2.081 | 68.580 | 1.00 | 40.99 |
| ATOM | 17122 | NH1 | ARG | 2555 | 41.356 | 0.836 | 68.666 | 1.00 | 41.89 |
| ATOM | 17123 | NH2 | ARG | 2555 | 41.075 | 3.079 | 69.058 | 1.00 | 38.37 |
| ATOM | 17124 | C | ARG | 2555 | 46.310 | −1.242 | 64.581 | 1.00 | 34.23 |
| ATOM | 17125 | O | ARG | 2555 | 45.958 | −2.208 | 63.903 | 1.00 | 33.15 |
| ATOM | 17126 | N | GLY | 2556 | 47.523 | −1.140 | 65.114 | 1.00 | 34.60 |
| ATOM | 17127 | CA | GLY | 2556 | 48.503 | −2.194 | 64.912 | 1.00 | 35.58 |
| ATOM | 17128 | C | GLY | 2556 | 49.469 | −1.893 | 63.784 | 1.00 | 35.76 |
| ATOM | 17129 | O | GLY | 2556 | 49.157 | −1.128 | 62.872 | 1.00 | 35.13 |
| ATOM | 17130 | N | ASP | 2557 | 50.647 | −2.505 | 63.839 | 1.00 | 36.48 |
| ATOM | 17131 | CA | ASP | 2557 | 51.663 | −2.282 | 62.819 | 1.00 | 36.98 |
| ATOM | 17132 | CB | ASP | 2557 | 52.984 | −2.932 | 63.231 | 1.00 | 37.65 |
| ATOM | 17133 | CG | ASP | 2557 | 53.604 | −2.265 | 64.437 | 1.00 | 38.08 |
| ATOM | 17134 | OD1 | ASP | 2557 | 53.271 | −1.088 | 64.697 | 1.00 | 38.16 |
| ATOM | 17135 | OD2 | ASP | 2557 | 54.434 | −2.909 | 65.115 | 1.00 | 38.66 |
| ATOM | 17136 | C | ASP | 2557 | 51.266 | −2.776 | 61.439 | 1.00 | 36.71 |
| ATOM | 17137 | O | ASP | 2557 | 51.405 | −2.049 | 60.457 | 1.00 | 38.15 |
| ATOM | 17138 | N | GLU | 2558 | 50.777 | −4.009 | 61.358 | 1.00 | 36.83 |
| ATOM | 17139 | CA | GLU | 2558 | 50.381 | −4.573 | 60.074 | 1.00 | 37.15 |
| ATOM | 17140 | CB | GLU | 2558 | 49.895 | −6.012 | 60.242 | 1.00 | 40.41 |
| ATOM | 17141 | CG | GLU | 2558 | 49.768 | −6.763 | 58.928 | 1.00 | 44.93 |
| ATOM | 17142 | CD | GLU | 2558 | 48.831 | −7.946 | 59.018 | 1.00 | 47.28 |
| ATOM | 17143 | OE1 | GLU | 2558 | 49.026 | −8.794 | 59.916 | 1.00 | 48.79 |
| ATOM | 17144 | OE2 | GLU | 2558 | 47.899 | −8.027 | 58.187 | 1.00 | 48.79 |
| ATOM | 17145 | C | GLU | 2558 | 49.270 | −3.740 | 59.450 | 1.00 | 36.06 |
| ATOM | 17146 | O | GLU | 2558 | 49.344 | −3.363 | 58.280 | 1.00 | 34.74 |
| ATOM | 17147 | N | ALA | 2559 | 48.239 | −3.456 | 60.240 | 1.00 | 33.89 |
| ATOM | 17148 | CA | ALA | 2559 | 47.114 | −2.665 | 59.763 | 1.00 | 32.57 |
| ATOM | 17149 | CB | ALA | 2559 | 46.037 | −2.571 | 60.846 | 1.00 | 32.43 |
| ATOM | 17150 | C | ALA | 2559 | 47.590 | −1.274 | 59.366 | 1.00 | 30.42 |
| ATOM | 17151 | O | ALA | 2559 | 47.180 | −0.739 | 58.338 | 1.00 | 30.62 |
| ATOM | 17152 | N | GLY | 2560 | 48.460 | −0.694 | 60.188 | 1.00 | 28.46 |
| ATOM | 17153 | CA | GLY | 2560 | 48.978 | 0.630 | 59.898 | 1.00 | 26.73 |
| ATOM | 17154 | C | GLY | 2560 | 49.817 | 0.672 | 58.633 | 1.00 | 25.98 |
| ATOM | 17155 | O | GLY | 2560 | 49.664 | 1.574 | 57.811 | 1.00 | 25.52 |
| ATOM | 17156 | N | ASP | 2561 | 50.703 | −0.305 | 58.465 | 1.00 | 25.18 |
| ATOM | 17157 | CA | ASP | 2561 | 51.558 | −0.339 | 57.285 | 1.00 | 24.81 |
| ATOM | 17158 | CB | ASP | 2561 | 52.603 | −1.451 | 57.401 | 1.00 | 26.12 |
| ATOM | 17159 | CG | ASP | 2561 | 53.606 | −1.197 | 58.507 | 1.00 | 26.29 |
| ATOM | 17160 | OD1 | ASP | 2561 | 53.951 | −0.020 | 58.744 | 1.00 | 27.90 |
| ATOM | 17161 | OD2 | ASP | 2561 | 54.064 | −2.178 | 59.127 | 1.00 | 29.88 |
| ATOM | 17162 | C | ASP | 2561 | 50.731 | −0.545 | 56.022 | 1.00 | 24.94 |
| ATOM | 17163 | O | ASP | 2561 | 51.032 | 0.020 | 54.967 | 1.00 | 25.26 |
| ATOM | 17164 | N | GLN | 2562 | 49.681 | −1.351 | 56.127 | 1.00 | 25.61 |
| ATOM | 17165 | CA | GLN | 2562 | 48.829 | −1.612 | 54.975 | 1.00 | 24.70 |
| ATOM | 17166 | CB | GLN | 2562 | 47.763 | −2.651 | 55.324 | 1.00 | 27.82 |
| ATOM | 17167 | CG | GLN | 2562 | 46.939 | −3.080 | 54.123 | 1.00 | 28.73 |
| ATOM | 17168 | CD | GLN | 2562 | 47.781 | −3.794 | 53.087 | 1.00 | 29.94 |
| ATOM | 17169 | OE1 | GLN | 2562 | 48.326 | −4.865 | 53.356 | 1.00 | 31.90 |
| ATOM | 17170 | NE2 | GLN | 2562 | 47.900 | −3.204 | 51.898 | 1.00 | 29.29 |
| ATOM | 17171 | C | GLN | 2562 | 48.157 | −0.327 | 54.504 | 1.00 | 24.50 |
| ATOM | 17172 | O | GLN | 2562 | 48.084 | −0.059 | 53.303 | 1.00 | 23.09 |
| ATOM | 17173 | N | LEU | 2563 | 47.658 | 0.468 | 55.448 | 1.00 | 25.26 |
| ATOM | 17174 | CA | LEU | 2563 | 46.999 | 1.728 | 55.102 | 1.00 | 24.24 |
| ATOM | 17175 | CB | LEU | 2563 | 46.331 | 2.353 | 56.333 | 1.00 | 24.36 |
| ATOM | 17176 | CG | LEU | 2563 | 45.074 | 1.675 | 56.882 | 1.00 | 26.38 |
| ATOM | 17177 | CD1 | LEU | 2563 | 44.544 | 2.465 | 58.070 | 1.00 | 27.10 |
| ATOM | 17178 | CD2 | LEU | 2563 | 44.018 | 1.599 | 55.797 | 1.00 | 27.72 |
| ATOM | 17179 | C | LEU | 2563 | 48.006 | 2.707 | 54.528 | 1.00 | 24.78 |
| ATOM | 17180 | O | LEU | 2563 | 47.694 | 3.476 | 53.619 | 1.00 | 22.15 |
| ATOM | 17181 | N | LEU | 2564 | 49.220 | 2.682 | 55.067 | 1.00 | 24.45 |
| ATOM | 17182 | CA | LEU | 2564 | 50.264 | 3.580 | 54.590 | 1.00 | 23.89 |
| ATOM | 17183 | CB | LEU | 2564 | 51.495 | 3.480 | 55.491 | 1.00 | 25.26 |
| ATOM | 17184 | CG | LEU | 2564 | 52.282 | 4.773 | 55.744 | 1.00 | 27.78 |
| ATOM | 17185 | CD1 | LEU | 2564 | 53.598 | 4.405 | 56.410 | 1.00 | 29.55 |
| ATOM | 17186 | CD2 | LEU | 2564 | 52.535 | 5.535 | 54.457 | 1.00 | 27.60 |
| ATOM | 17187 | C | LEU | 2564 | 50.638 | 3.173 | 53.169 | 1.00 | 21.75 |
| ATOM | 17188 | O | LEU | 2564 | 50.738 | 4.013 | 52.277 | 1.00 | 22.25 |
| ATOM | 17189 | N | SER | 2565 | 50.838 | 1.875 | 52.968 | 1.00 | 21.52 |
| ATOM | 17190 | CA | SER | 2565 | 51.205 | 1.361 | 51.658 | 1.00 | 23.48 |
| ATOM | 17191 | CB | SER | 2565 | 51.473 | −0.142 | 51.745 | 1.00 | 26.47 |
| ATOM | 17192 | OG | SER | 2565 | 51.998 | −0.634 | 50.523 | 1.00 | 34.86 |
| ATOM | 17193 | C | SER | 2565 | 50.107 | 1.645 | 50.632 | 1.00 | 22.74 |
| ATOM | 17194 | O | SER | 2565 | 50.390 | 1.989 | 49.481 | 1.00 | 21.74 |
| ATOM | 17195 | N | ASP | 2566 | 48.853 | 1.500 | 51.054 | 1.00 | 19.93 |
| ATOM | 17196 | CA | ASP | 2566 | 47.718 | 1.753 | 50.168 | 1.00 | 18.63 |
| ATOM | 17197 | CB | ASP | 2566 | 46.399 | 1.335 | 50.836 | 1.00 | 19.80 |
| ATOM | 17198 | CG | ASP | 2566 | 46.206 | −0.174 | 50.867 | 1.00 | 21.76 |
| ATOM | 17199 | OD1 | ASP | 2566 | 46.935 | −0.883 | 50.146 | 1.00 | 24.30 |
| ATOM | 17200 | OD2 | ASP | 2566 | 45.313 | −0.655 | 51.605 | 1.00 | 20.99 |
| ATOM | 17201 | C | ASP | 2566 | 47.647 | 3.226 | 49.785 | 1.00 | 18.77 |
| ATOM | 17202 | O | ASP | 2566 | 47.329 | 3.560 | 48.650 | 1.00 | 19.96 |
| ATOM | 17203 | N | ALA | 2567 | 47.945 | 4.109 | 50.732 | 1.00 | 17.84 |
| ATOM | 17204 | CA | ALA | 2567 | 47.904 | 5.536 | 50.451 | 1.00 | 17.61 |
| ATOM | 17205 | CB | ALA | 2567 | 48.135 | 6.341 | 51.731 | 1.00 | 18.06 |
| ATOM | 17206 | C | ALA | 2567 | 48.964 | 5.894 | 49.412 | 1.00 | 16.53 |
| ATOM | 17207 | O | ALA | 2567 | 48.705 | 6.675 | 48.504 | 1.00 | 15.81 |
| ATOM | 17208 | N | LEU | 2568 | 50.162 | 5.338 | 49.560 | 1.00 | 16.61 |
| ATOM | 17209 | CA | LEU | 2568 | 51.232 | 5.615 | 48.613 | 1.00 | 18.69 |
| ATOM | 17210 | CB | LEU | 2568 | 52.563 | 5.047 | 49.127 | 1.00 | 21.36 |
| ATOM | 17211 | CG | LEU | 2568 | 53.121 | 5.741 | 50.379 | 1.00 | 22.49 |
| ATOM | 17212 | CD1 | LEU | 2568 | 54.332 | 4.997 | 50.913 | 1.00 | 25.07 |
| ATOM | 17213 | CD2 | LEU | 2568 | 53.484 | 7.182 | 50.031 | 1.00 | 24.13 |
| ATOM | 17214 | C | LEU | 2568 | 50.887 | 5.011 | 47.249 | 1.00 | 18.91 |
| ATOM | 17215 | O | LEU | 2568 | 51.158 | 5.611 | 46.214 | 1.00 | 20.34 |
| ATOM | 17216 | N | ALA | 2569 | 50.268 | 3.835 | 47.254 | 1.00 | 19.41 |
| ATOM | 17217 | CA | ALA | 2569 | 49.892 | 3.155 | 46.015 | 1.00 | 20.41 |
| ATOM | 17218 | CB | ALA | 2569 | 49.395 | 1.738 | 46.317 | 1.00 | 19.39 |
| ATOM | 17219 | C | ALA | 2569 | 48.820 | 3.929 | 45.257 | 1.00 | 20.57 |
| ATOM | 17220 | O | ALA | 2569 | 48.834 | 3.982 | 44.024 | 1.00 | 18.66 |
| ATOM | 17221 | N | LEU | 2570 | 47.875 | 4.506 | 45.996 | 1.00 | 21.33 |
| ATOM | 17222 | CA | LEU | 2570 | 46.810 | 5.295 | 45.385 | 1.00 | 20.35 |
| ATOM | 17223 | CB | LEU | 2570 | 45.718 | 5.630 | 46.422 | 1.00 | 20.88 |
| ATOM | 17224 | CG | LEU | 2570 | 44.822 | 4.476 | 46.878 | 1.00 | 20.36 |
| ATOM | 17225 | CD1 | LEU | 2570 | 43.854 | 4.960 | 47.959 | 1.00 | 20.64 |
| ATOM | 17226 | CD2 | LEU | 2570 | 44.056 | 3.926 | 45.682 | 1.00 | 21.94 |
| ATOM | 17227 | C | LEU | 2570 | 47.401 | 6.582 | 44.824 | 1.00 | 20.57 |
| ATOM | 17228 | O | LEU | 2570 | 46.999 | 7.049 | 43.754 | 1.00 | 16.60 |
| ATOM | 17229 | N | GLU | 2571 | 48.356 | 7.163 | 45.547 | 1.00 | 19.59 |
| ATOM | 17230 | CA | GLU | 2571 | 48.990 | 8.389 | 45.076 | 1.00 | 20.08 |
| ATOM | 17231 | CB | GLU | 2571 | 49.965 | 8.937 | 46.127 | 1.00 | 19.80 |
| ATOM | 17232 | CG | GLU | 2571 | 50.705 | 10.184 | 45.662 | 1.00 | 22.18 |
| ATOM | 17233 | CD | GLU | 2571 | 51.688 | 10.684 | 46.706 | 1.00 | 22.20 |
| ATOM | 17234 | OE1 | GLU | 2571 | 52.470 | 9.854 | 47.213 | 1.00 | 23.24 |
| ATOM | 17235 | OE2 | GLU | 2571 | 51.677 | 11.891 | 47.003 | 1.00 | 21.21 |
| ATOM | 17236 | C | GLU | 2571 | 49.748 | 8.124 | 43.779 | 1.00 | 19.41 |
| ATOM | 17237 | O | GLU | 2571 | 49.658 | 8.904 | 42.833 | 1.00 | 20.18 |
| ATOM | 17238 | N | ALA | 2572 | 50.491 | 7.020 | 43.745 | 1.00 | 20.49 |
| ATOM | 17239 | CA | ALA | 2572 | 51.266 | 6.660 | 42.564 | 1.00 | 21.39 |
| ATOM | 17240 | CB | ALA | 2572 | 52.137 | 5.453 | 42.859 | 1.00 | 23.82 |
| ATOM | 17241 | C | ALA | 2572 | 50.347 | 6.363 | 41.388 | 1.00 | 22.09 |
| ATOM | 17242 | O | ALA | 2572 | 50.718 | 6.555 | 40.228 | 1.00 | 20.31 |
| ATOM | 17243 | N | ALA | 2573 | 49.142 | 5.903 | 41.702 | 1.00 | 22.04 |
| ATOM | 17244 | CA | ALA | 2573 | 48.152 | 5.564 | 40.689 | 1.00 | 21.67 |
| ATOM | 17245 | CB | ALA | 2573 | 47.038 | 4.725 | 41.313 | 1.00 | 20.41 |
| ATOM | 17246 | C | ALA | 2573 | 47.569 | 6.807 | 40.029 | 1.00 | 20.80 |
| ATOM | 17247 | O | ALA | 2573 | 47.084 | 6.746 | 38.900 | 1.00 | 20.43 |
| ATOM | 17248 | N | GLY | 2574 | 47.615 | 7.934 | 40.733 | 1.00 | 19.07 |
| ATOM | 17249 | CA | GLY | 2574 | 47.089 | 9.163 | 40.168 | 1.00 | 17.50 |
| ATOM | 17250 | C | GLY | 2574 | 46.141 | 9.944 | 41.060 | 1.00 | 17.16 |
| ATOM | 17251 | O | GLY | 2574 | 45.716 | 11.041 | 40.701 | 1.00 | 17.95 |
| ATOM | 17252 | N | ALA | 2575 | 45.796 | 9.390 | 42.217 | 1.00 | 17.09 |
| ATOM | 17253 | CA | ALA | 2575 | 44.904 | 10.083 | 43.137 | 1.00 | 19.39 |
| ATOM | 17254 | CB | ALA | 2575 | 44.694 | 9.246 | 44.388 | 1.00 | 17.57 |
| ATOM | 17255 | C | ALA | 2575 | 45.516 | 11.444 | 43.498 | 1.00 | 20.74 |
| ATOM | 17256 | O | ALA | 2575 | 46.687 | 11.526 | 43.876 | 1.00 | 22.35 |
| ATOM | 17257 | N | GLN | 2576 | 44.731 | 12.510 | 43.375 | 1.00 | 20.06 |
| ATOM | 17258 | CA | GLN | 2576 | 45.226 | 13.848 | 43.684 | 1.00 | 21.20 |
| ATOM | 17259 | CB | GLN | 2576 | 44.733 | 14.836 | 42.628 | 1.00 | 24.45 |
| ATOM | 17260 | CG | GLN | 2576 | 45.226 | 14.509 | 41.223 | 1.00 | 29.02 |
| ATOM | 17261 | CD | GLN | 2576 | 44.551 | 15.337 | 40.149 | 1.00 | 31.24 |
| ATOM | 17262 | OE1 | GLN | 2576 | 44.686 | 16.559 | 40.109 | 1.00 | 36.20 |
| ATOM | 17263 | NE2 | GLN | 2576 | 43.811 | 14.671 | 39.271 | 1.00 | 32.65 |
| ATOM | 17264 | C | GLN | 2576 | 44.822 | 14.316 | 45.082 | 1.00 | 21.51 |
| ATOM | 17265 | O | GLN | 2576 | 45.260 | 15.371 | 45.549 | 1.00 | 19.45 |
| ATOM | 17266 | N | LEU | 2577 | 43.991 | 13.519 | 45.744 | 1.00 | 20.67 |
| ATOM | 17267 | CA | LEU | 2577 | 43.517 | 13.817 | 47.093 | 1.00 | 21.57 |
| ATOM | 17268 | CB | LEU | 2577 | 42.207 | 14.609 | 47.032 | 1.00 | 25.05 |
| ATOM | 17269 | CG | LEU | 2577 | 42.283 | 16.133 | 47.063 | 1.00 | 26.27 |
| ATOM | 17270 | CD1 | LEU | 2577 | 40.924 | 16.728 | 46.738 | 1.00 | 27.14 |
| ATOM | 17271 | CD2 | LEU | 2577 | 42.751 | 16.576 | 48.449 | 1.00 | 27.72 |
| ATOM | 17272 | C | LEU | 2577 | 43.280 | 12.515 | 47.850 | 1.00 | 21.36 |
| ATOM | 17273 | O | LEU | 2577 | 43.059 | 11.472 | 47.239 | 1.00 | 22.18 |
| ATOM | 17274 | N | LEU | 2578 | 43.327 | 12.571 | 49.178 | 1.00 | 19.22 |
| ATOM | 17275 | CA | LEU | 2578 | 43.085 | 11.383 | 49.991 | 1.00 | 19.03 |
| ATOM | 17276 | CB | LEU | 2578 | 44.403 | 10.691 | 50.389 | 1.00 | 19.80 |
| ATOM | 17277 | CG | LEU | 2578 | 44.235 | 9.475 | 51.320 | 1.00 | 20.24 |
| ATOM | 17278 | CD1 | LEU | 2578 | 43.537 | 8.346 | 50.556 | 1.00 | 21.15 |
| ATOM | 17279 | CD2 | LEU | 2578 | 45.587 | 8.989 | 51.840 | 1.00 | 20.62 |
| ATOM | 17280 | C | LEU | 2578 | 42.321 | 11.722 | 51.261 | 1.00 | 20.13 |
| ATOM | 17281 | O | LEU | 2578 | 42.636 | 12.694 | 51.949 | 1.00 | 20.75 |
| ATOM | 17282 | N | VAL | 2579 | 41.303 | 10.924 | 51.560 | 1.00 | 19.83 |
| ATOM | 17283 | CA | VAL | 2579 | 40.529 | 11.105 | 52.779 | 1.00 | 18.16 |
| ATOM | 17284 | CB | VAL | 2579 | 39.000 | 11.083 | 52.508 | 1.00 | 17.51 |
| ATOM | 17285 | CG1 | VAL | 2579 | 38.230 | 10.860 | 53.822 | 1.00 | 16.02 |
| ATOM | 17286 | CG2 | VAL | 2579 | 38.569 | 12.401 | 51.890 | 1.00 | 17.32 |
| ATOM | 17287 | C | VAL | 2579 | 40.884 | 9.962 | 53.720 | 1.00 | 19.66 |
| ATOM | 17288 | O | VAL | 2579 | 40.874 | 8.793 | 53.321 | 1.00 | 19.81 |
| ATOM | 17289 | N | LEU | 2580 | 41.232 | 10.316 | 54.956 | 1.00 | 19.69 |
| ATOM | 17290 | CA | LEU | 2580 | 41.576 | 9.350 | 55.997 | 1.00 | 20.23 |
| ATOM | 17291 | CB | LEU | 2580 | 42.912 | 9.684 | 56.656 | 1.00 | 21.71 |
| ATOM | 17292 | CG | LEU | 2580 | 44.174 | 9.119 | 56.045 | 1.00 | 24.93 |
| ATOM | 17293 | CD1 | LEU | 2580 | 45.332 | 9.429 | 56.983 | 1.00 | 22.17 |
| ATOM | 17294 | CD2 | LEU | 2580 | 44.027 | 7.607 | 55.848 | 1.00 | 22.55 |
| ATOM | 17295 | C | LEU | 2580 | 40.510 | 9.440 | 57.063 | 1.00 | 19.52 |
| ATOM | 17296 | O | LEU | 2580 | 40.321 | 10.502 | 57.648 | 1.00 | 20.28 |
| ATOM | 17297 | N | GLU | 2581 | 39.840 | 8.327 | 57.330 | 1.00 | 19.37 |
| ATOM | 17298 | CA | GLU | 2581 | 38.759 | 8.310 | 58.308 | 1.00 | 19.88 |
| ATOM | 17299 | CB | GLU | 2581 | 37.452 | 7.906 | 57.611 | 1.00 | 20.26 |
| ATOM | 17300 | CG | GLU | 2581 | 36.289 | 7.660 | 58.564 | 1.00 | 22.54 |
| ATOM | 17301 | CD | GLU | 2581 | 34.945 | 7.737 | 57.869 | 1.00 | 24.53 |
| ATOM | 17302 | OE1 | GLU | 2581 | 34.920 | 7.787 | 56.622 | 1.00 | 21.82 |
| ATOM | 17303 | OE2 | GLU | 2581 | 33.912 | 7.748 | 58.572 | 1.00 | 26.36 |
| ATOM | 17304 | C | GLU | 2581 | 38.974 | 7.416 | 59.519 | 1.00 | 17.68 |
| ATOM | 17305 | O | GLU | 2581 | 39.317 | 6.247 | 59.388 | 1.00 | 18.36 |
| ATOM | 17306 | N | CYS | 2582 | 38.779 | 7.988 | 60.701 | 1.00 | 19.70 |
| ATOM | 17307 | CA | CYS | 2582 | 38.896 | 7.263 | 61.955 | 1.00 | 19.88 |
| ATOM | 17308 | CB | CYS | 2582 | 37.605 | 6.489 | 62.218 | 1.00 | 21.29 |
| ATOM | 17309 | SG | CYS | 2582 | 36.197 | 7.601 | 62.460 | 1.00 | 24.19 |
| ATOM | 17310 | C | CYS | 2582 | 40.091 | 6.340 | 62.067 | 1.00 | 19.95 |
| ATOM | 17311 | O | CYS | 2582 | 39.963 | 5.118 | 62.120 | 1.00 | 21.49 |
| ATOM | 17312 | N | VAL | 2583 | 41.261 | 6.954 | 62.127 | 1.00 | 21.05 |
| ATOM | 17313 | CA | VAL | 2583 | 42.506 | 6.226 | 62.241 | 1.00 | 23.03 |
| ATOM | 17314 | CB | VAL | 2583 | 43.317 | 6.351 | 60.924 | 1.00 | 23.48 |
| ATOM | 17315 | CG1 | VAL | 2583 | 43.590 | 7.812 | 60.617 | 1.00 | 23.25 |
| ATOM | 17316 | CG2 | VAL | 2583 | 44.609 | 5.593 | 61.029 | 1.00 | 25.06 |
| ATOM | 17317 | C | VAL | 2583 | 43.277 | 6.871 | 63.385 | 1.00 | 23.22 |
| ATOM | 17318 | O | VAL | 2583 | 43.094 | 8.053 | 63.675 | 1.00 | 22.61 |
| ATOM | 17319 | N | PRO | 2584 | 44.132 | 6.099 | 64.067 | 1.00 | 24.47 |
| ATOM | 17320 | CD | PRO | 2584 | 44.412 | 4.662 | 63.956 | 1.00 | 23.45 |
| ATOM | 17321 | CA | PRO | 2584 | 44.891 | 6.701 | 65.163 | 1.00 | 24.14 |
| ATOM | 17322 | CB | PRO | 2584 | 45.745 | 5.538 | 65.680 | 1.00 | 25.70 |
| ATOM | 17323 | CG | PRO | 2584 | 45.792 | 4.581 | 64.518 | 1.00 | 28.17 |
| ATOM | 17324 | C | PRO | 2584 | 45.708 | 7.873 | 64.635 | 1.00 | 24.60 |
| ATOM | 17325 | O | PRO | 2584 | 46.285 | 7.804 | 63.545 | 1.00 | 23.19 |
| ATOM | 17326 | N | VAL | 2585 | 45.727 | 8.958 | 65.400 | 1.00 | 23.51 |
| ATOM | 17327 | CA | VAL | 2585 | 46.444 | 10.172 | 65.025 | 1.00 | 23.75 |
| ATOM | 17328 | CB | VAL | 2585 | 46.511 | 11.158 | 66.214 | 1.00 | 24.99 |
| ATOM | 17329 | CG1 | VAL | 2585 | 47.098 | 12.491 | 65.758 | 1.00 | 23.50 |
| ATOM | 17330 | CG2 | VAL | 2585 | 45.124 | 11.363 | 66.790 | 1.00 | 25.53 |
| ATOM | 17331 | C | VAL | 2585 | 47.865 | 9.895 | 64.549 | 1.00 | 25.46 |
| ATOM | 17332 | O | VAL | 2585 | 48.317 | 10.477 | 63.563 | 1.00 | 24.79 |
| ATOM | 17333 | N | GLU | 2586 | 48.563 | 9.002 | 65.245 | 1.00 | 26.45 |
| ATOM | 17334 | CA | GLU | 2586 | 49.939 | 8.683 | 64.879 | 1.00 | 28.54 |
| ATOM | 17335 | CB | GLU | 2586 | 50.559 | 7.688 | 65.873 | 1.00 | 30.14 |
| ATOM | 17336 | CG | GLU | 2586 | 49.580 | 6.852 | 66.695 | 1.00 | 35.51 |
| ATOM | 17337 | CD | GLU | 2586 | 48.843 | 7.665 | 67.757 | 1.00 | 37.22 |
| ATOM | 17338 | OE1 | GLU | 2586 | 49.464 | 8.566 | 68.363 | 1.00 | 38.20 |
| ATOM | 17339 | OE2 | GLU | 2586 | 47.649 | 7.388 | 67.992 | 1.00 | 39.25 |
| ATOM | 17340 | C | GLU | 2586 | 50.064 | 8.150 | 63.456 | 1.00 | 28.46 |
| ATOM | 17341 | O | GLU | 2586 | 51.045 | 8.425 | 62.769 | 1.00 | 27.67 |
| ATOM | 17342 | N | LEU | 2587 | 49.065 | 7.394 | 63.017 | 1.00 | 27.65 |
| ATOM | 17343 | CA | LEU | 2587 | 49.077 | 6.834 | 61.674 | 1.00 | 26.73 |
| ATOM | 17344 | CB | LEU | 2587 | 48.026 | 5.731 | 61.554 | 1.00 | 28.21 |
| ATOM | 17345 | CG | LEU | 2587 | 48.378 | 4.497 | 60.721 | 1.00 | 30.72 |
| ATOM | 17346 | CD1 | LEU | 2587 | 47.099 | 3.720 | 60.454 | 1.00 | 29.14 |
| ATOM | 17347 | CD2 | LEU | 2587 | 49.043 | 4.886 | 59.409 | 1.00 | 30.86 |
| ATOM | 17348 | C | LEU | 2587 | 48.785 | 7.937 | 60.654 | 1.00 | 26.34 |
| ATOM | 17349 | O | LEU | 2587 | 49.398 | 7.987 | 59.583 | 1.00 | 24.48 |
| ATOM | 17350 | N | ALA | 2588 | 47.848 | 8.820 | 60.994 | 1.00 | 24.00 |
| ATOM | 17351 | CA | ALA | 2588 | 47.484 | 9.922 | 60.111 | 1.00 | 23.70 |
| ATOM | 17352 | CB | ALA | 2588 | 46.331 | 10.737 | 60.712 | 1.00 | 23.08 |
| ATOM | 17353 | C | ALA | 2588 | 48.691 | 10.815 | 59.887 | 1.00 | 24.14 |
| ATOM | 17354 | O | ALA | 2588 | 48.839 | 11.415 | 58.822 | 1.00 | 22.54 |
| ATOM | 17355 | N | LYS | 2589 | 49.548 | 10.904 | 60.901 | 1.00 | 24.22 |
| ATOM | 17356 | CA | LYS | 2589 | 50.760 | 11.709 | 60.816 | 1.00 | 25.74 |
| ATOM | 17357 | CB | LYS | 2589 | 51.471 | 11.751 | 62.172 | 1.00 | 27.20 |
| ATOM | 17358 | CG | LYS | 2589 | 50.586 | 12.130 | 63.345 | 1.00 | 32.13 |
| ATOM | 17359 | CD | LYS | 2589 | 51.399 | 12.323 | 64.625 | 1.00 | 36.29 |
| ATOM | 17360 | CE | LYS | 2589 | 50.510 | 12.760 | 65.784 | 1.00 | 35.55 |
| ATOM | 17361 | NZ | LYS | 2589 | 51.296 | 13.309 | 66.917 | 1.00 | 39.50 |
| ATOM | 17362 | C | LYS | 2589 | 51.694 | 11.086 | 59.781 | 1.00 | 23.37 |
| ATOM | 17363 | O | LYS | 2589 | 52.171 | 11.768 | 58.881 | 1.00 | 26.48 |
| ATOM | 17364 | N | ARG | 2590 | 51.934 | 9.785 | 59.915 | 1.00 | 23.58 |
| ATOM | 17365 | CA | ARG | 2590 | 52.813 | 9.062 | 59.005 | 1.00 | 22.93 |
| ATOM | 17366 | CB | ARG | 2590 | 52.882 | 7.575 | 59.374 | 1.00 | 25.56 |
| ATOM | 17367 | CG | ARG | 2590 | 52.959 | 7.280 | 60.863 | 1.00 | 27.56 |
| ATOM | 17368 | CD | ARG | 2590 | 53.951 | 6.177 | 61.187 | 1.00 | 28.90 |
| ATOM | 17369 | NE | ARG | 2590 | 53.876 | 4.999 | 60.319 | 1.00 | 28.63 |
| ATOM | 17370 | CZ | ARG | 2590 | 53.110 | 3.933 | 60.536 | 1.00 | 30.48 |
| ATOM | 17371 | NH1 | ARG | 2590 | 52.322 | 3.872 | 61.601 | 1.00 | 30.99 |
| ATOM | 17372 | NH2 | ARG | 2590 | 53.159 | 2.903 | 59.701 | 1.00 | 30.22 |
| ATOM | 17373 | C | ARG | 2590 | 52.346 | 9.183 | 57.556 | 1.00 | 22.50 |
| ATOM | 17374 | O | ARG | 2590 | 53.152 | 9.366 | 56.641 | 1.00 | 21.17 |
| ATOM | 17375 | N | ILE | 2591 | 51.040 | 9.073 | 57.342 | 1.00 | 20.92 |
| ATOM | 17376 | CA | ILE | 2591 | 50.493 | 9.150 | 55.993 | 1.00 | 19.61 |
| ATOM | 17377 | CB | ILE | 2591 | 49.023 | 8.673 | 55.980 | 1.00 | 20.34 |
| ATOM | 17378 | CG2 | ILE | 2591 | 48.418 | 8.871 | 54.600 | 1.00 | 21.46 |
| ATOM | 17379 | CG1 | ILE | 2591 | 48.971 | 7.206 | 56.395 | 1.00 | 20.56 |
| ATOM | 17380 | CD1 | ILE | 2591 | 47.585 | 6.658 | 56.588 | 1.00 | 25.52 |
| ATOM | 17381 | C | ILE | 2591 | 50.591 | 10.552 | 55.410 | 1.00 | 20.03 |
| ATOM | 17382 | O | ILE | 2591 | 51.005 | 10.732 | 54.261 | 1.00 | 19.07 |
| ATOM | 17383 | N | THR | 2592 | 50.225 | 11.549 | 56.207 | 1.00 | 20.25 |
| ATOM | 17384 | CA | THR | 2592 | 50.276 | 12.928 | 55.749 | 1.00 | 22.08 |
| ATOM | 17385 | CB | THR | 2592 | 49.743 | 13.894 | 56.826 | 1.00 | 21.69 |
| ATOM | 17386 | OG1 | THR | 2592 | 48.358 | 13.615 | 57.070 | 1.00 | 22.71 |
| ATOM | 17387 | CG2 | THR | 2592 | 49.889 | 15.343 | 56.368 | 1.00 | 22.53 |
| ATOM | 17388 | C | THR | 2592 | 51.693 | 13.346 | 55.376 | 1.00 | 23.76 |
| ATOM | 17389 | O | THR | 2592 | 51.896 | 14.092 | 54.417 | 1.00 | 24.12 |
| ATOM | 17390 | N | GLU | 2593 | 52.676 | 12.871 | 56.129 | 1.00 | 25.91 |
| ATOM | 17391 | CA | GLU | 2593 | 54.059 | 13.227 | 55.834 | 1.00 | 27.67 |
| ATOM | 17392 | CB | GLU | 2593 | 54.936 | 13.023 | 57.071 | 1.00 | 29.33 |
| ATOM | 17393 | CG | GLU | 2593 | 54.427 | 13.761 | 58.299 | 1.00 | 33.19 |
| ATOM | 17394 | CD | GLU | 2593 | 55.367 | 13.650 | 59.486 | 1.00 | 36.93 |
| ATOM | 17395 | OE1 | GLU | 2593 | 55.815 | 12.520 | 59.792 | 1.00 | 38.76 |
| ATOM | 17396 | OE2 | GLU | 2593 | 55.649 | 14.689 | 60.121 | 1.00 | 38.10 |
| ATOM | 17397 | C | GLU | 2593 | 54.600 | 12.407 | 54.664 | 1.00 | 26.14 |
| ATOM | 17398 | O | GLU | 2593 | 55.381 | 12.911 | 53.855 | 1.00 | 26.29 |
| ATOM | 17399 | N | ALA | 2594 | 54.172 | 11.153 | 54.565 | 1.00 | 24.55 |
| ATOM | 17400 | CA | ALA | 2594 | 54.626 | 10.281 | 53.489 | 1.00 | 24.61 |
| ATOM | 17401 | CB | ALA | 2594 | 54.252 | 8.833 | 53.801 | 1.00 | 24.77 |
| ATOM | 17402 | C | ALA | 2594 | 54.088 | 10.679 | 52.115 | 1.00 | 24.72 |
| ATOM | 17403 | O | ALA | 2594 | 54.781 | 10.519 | 51.106 | 1.00 | 24.92 |
| ATOM | 17404 | N | LEU | 2595 | 52.862 | 11.198 | 52.069 | 1.00 | 23.86 |
| ATOM | 17405 | CA | LEU | 2595 | 52.259 | 11.602 | 50.796 | 1.00 | 23.39 |
| ATOM | 17406 | CB | LEU | 2595 | 50.739 | 11.377 | 50.818 | 1.00 | 23.16 |
| ATOM | 17407 | CG | LEU | 2595 | 50.211 | 9.935 | 50.851 | 1.00 | 25.00 |
| ATOM | 17408 | CD1 | LEU | 2595 | 48.681 | 9.954 | 50.756 | 1.00 | 24.60 |
| ATOM | 17409 | CD2 | LEU | 2595 | 50.781 | 9.145 | 49.698 | 1.00 | 27.49 |
| ATOM | 17410 | C | LEU | 2595 | 52.526 | 13.050 | 50.410 | 1.00 | 22.38 |
| ATOM | 17411 | O | LEU | 2595 | 52.590 | 13.934 | 51.260 | 1.00 | 22.82 |
| ATOM | 17412 | N | ALA | 2596 | 52.673 | 13.280 | 49.112 | 1.00 | 21.91 |
| ATOM | 17413 | CA | ALA | 2596 | 52.907 | 14.618 | 48.580 | 1.00 | 20.98 |
| ATOM | 17414 | CB | ALA | 2596 | 53.676 | 14.532 | 47.259 | 1.00 | 21.84 |
| ATOM | 17415 | C | ALA | 2596 | 51.551 | 15.284 | 48.358 | 1.00 | 21.83 |
| ATOM | 17416 | O | ALA | 2596 | 51.402 | 16.500 | 48.522 | 1.00 | 19.07 |
| ATOM | 17417 | N | ILE | 2597 | 50.563 | 14.477 | 47.972 | 1.00 | 22.36 |
| ATOM | 17418 | CA | ILE | 2597 | 49.206 | 14.975 | 47.744 | 1.00 | 22.38 |
| ATOM | 17419 | CB | ILE | 2597 | 48.329 | 13.917 | 47.038 | 1.00 | 20.84 |
| ATOM | 17420 | CG2 | ILE | 2597 | 48.874 | 13.629 | 45.652 | 1.00 | 21.38 |
| ATOM | 17421 | CG1 | ILE | 2597 | 48.274 | 12.642 | 47.887 | 1.00 | 20.61 |
| ATOM | 17422 | CD1 | ILE | 2597 | 47.252 | 11.612 | 47.410 | 1.00 | 22.88 |
| ATOM | 17423 | C | ILE | 2597 | 48.556 | 15.315 | 49.084 | 1.00 | 23.26 |
| ATOM | 17424 | O | ILE | 2597 | 48.889 | 14.724 | 50.109 | 1.00 | 24.06 |
| ATOM | 17425 | N | PRO | 2598 | 47.618 | 16.276 | 49.091 | 1.00 | 24.26 |
| ATOM | 17426 | CD | PRO | 2598 | 47.190 | 17.134 | 47.973 | 1.00 | 24.78 |
| ATOM | 17427 | CA | PRO | 2598 | 46.951 | 16.657 | 50.339 | 1.00 | 24.43 |
| ATOM | 17428 | CB | PRO | 2598 | 46.158 | 17.908 | 49.946 | 1.00 | 24.29 |
| ATOM | 17429 | CG | PRO | 2598 | 45.908 | 17.716 | 48.487 | 1.00 | 27.18 |
| ATOM | 17430 | C | PRO | 2598 | 46.079 | 15.553 | 50.935 | 1.00 | 23.63 |
| ATOM | 17431 | O | PRO | 2598 | 45.401 | 14.813 | 50.218 | 1.00 | 23.16 |
| ATOM | 17432 | N | VAL | 2599 | 46.126 | 15.440 | 52.257 | 1.00 | 22.78 |
| ATOM | 17433 | CA | VAL | 2599 | 45.360 | 14.441 | 52.984 | 1.00 | 22.33 |
| ATOM | 17434 | CB | VAL | 2599 | 46.286 | 13.580 | 53.873 | 1.00 | 22.48 |
| ATOM | 17435 | CG1 | VAL | 2599 | 45.475 | 12.535 | 54.623 | 1.00 | 22.67 |
| ATOM | 17436 | CG2 | VAL | 2599 | 47.350 | 12.915 | 53.013 | 1.00 | 22.46 |
| ATOM | 17437 | C | VAL | 2599 | 44.311 | 15.133 | 53.857 | 1.00 | 23.71 |
| ATOM | 17438 | O | VAL | 2599 | 44.638 | 15.979 | 54.692 | 1.00 | 22.13 |
| ATOM | 17439 | N | ILE | 2600 | 43.048 | 14.779 | 53.642 | 1.00 | 21.85 |
| ATOM | 17440 | CA | ILE | 2600 | 41.944 | 15.360 | 54.397 | 1.00 | 20.33 |
| ATOM | 17441 | CB | ILE | 2600 | 40.774 | 15.719 | 53.459 | 1.00 | 19.87 |
| ATOM | 17442 | CG2 | ILE | 2600 | 39.599 | 16.283 | 54.269 | 1.00 | 20.69 |
| ATOM | 17443 | CG1 | ILE | 2600 | 41.265 | 16.720 | 52.409 | 1.00 | 22.41 |
| ATOM | 17444 | CD1 | ILE | 2600 | 40.294 | 16.981 | 51.262 | 1.00 | 23.07 |
| ATOM | 17445 | C | ILE | 2600 | 41.492 | 14.343 | 55.429 | 1.00 | 19.49 |
| ATOM | 17446 | O | ILE | 2600 | 41.199 | 13.199 | 55.094 | 1.00 | 18.22 |
| ATOM | 17447 | N | GLY | 2601 | 41.442 | 14.754 | 56.690 | 1.00 | 19.36 |
| ATOM | 17448 | CA | GLY | 2601 | 41.053 | 13.813 | 57.715 | 1.00 | 18.13 |
| ATOM | 17449 | C | GLY | 2601 | 39.700 | 14.052 | 58.342 | 1.00 | 18.77 |
| ATOM | 17450 | O | GLY | 2601 | 39.154 | 15.155 | 58.303 | 1.00 | 18.02 |
| ATOM | 17451 | N | ILE | 2602 | 39.155 | 12.976 | 58.892 | 1.00 | 18.53 |
| ATOM | 17452 | CA | ILE | 2602 | 37.892 | 12.998 | 59.600 | 1.00 | 19.83 |
| ATOM | 17453 | CB | ILE | 2602 | 36.684 | 12.679 | 58.671 | 1.00 | 21.77 |
| ATOM | 17454 | CG2 | ILE | 2602 | 36.964 | 11.449 | 57.822 | 1.00 | 21.95 |
| ATOM | 17455 | CG1 | ILE | 2602 | 35.424 | 12.479 | 59.518 | 1.00 | 24.57 |
| ATOM | 17456 | CD1 | ILE | 2602 | 35.090 | 13.650 | 60.403 | 1.00 | 27.84 |
| ATOM | 17457 | C | ILE | 2602 | 38.059 | 11.919 | 60.658 | 1.00 | 19.03 |
| ATOM | 17458 | O | ILE | 2602 | 38.075 | 10.724 | 60.354 | 1.00 | 20.33 |
| ATOM | 17459 | N | GLY | 2603 | 38.217 | 12.352 | 61.901 | 1.00 | 16.91 |
| ATOM | 17460 | CA | GLY | 2603 | 38.431 | 11.411 | 62.980 | 1.00 | 18.24 |
| ATOM | 17461 | C | GLY | 2603 | 39.864 | 10.914 | 62.930 | 1.00 | 18.71 |
| ATOM | 17462 | O | GLY | 2603 | 40.169 | 9.825 | 63.410 | 1.00 | 19.69 |
| ATOM | 17463 | N | ALA | 2604 | 40.743 | 11.717 | 62.335 | 1.00 | 20.83 |
| ATOM | 17464 | CA | ALA | 2604 | 42.160 | 11.363 | 62.210 | 1.00 | 22.46 |
| ATOM | 17465 | CB | ALA | 2604 | 42.534 | 11.229 | 60.735 | 1.00 | 21.76 |
| ATOM | 17466 | C | ALA | 2604 | 43.076 | 12.382 | 62.885 | 1.00 | 23.51 |
| ATOM | 17467 | O | ALA | 2604 | 44.292 | 12.370 | 62.671 | 1.00 | 23.26 |
| ATOM | 17468 | N | GLY | 2605 | 42.492 | 13.268 | 63.689 | 1.00 | 22.78 |
| ATOM | 17469 | CA | GLY | 2605 | 43.285 | 14.273 | 64.381 | 1.00 | 24.37 |
| ATOM | 17470 | C | GLY | 2605 | 43.540 | 15.523 | 63.560 | 1.00 | 23.63 |
| ATOM | 17471 | O | GLY | 2605 | 43.042 | 15.650 | 62.444 | 1.00 | 23.74 |
| ATOM | 17472 | N | ASN | 2606 | 44.322 | 16.453 | 64.102 | 1.00 | 22.37 |
| ATOM | 17473 | CA | ASN | 2606 | 44.610 | 17.696 | 63.391 | 1.00 | 22.84 |
| ATOM | 17474 | CB | ASN | 2606 | 44.614 | 18.884 | 64.368 | 1.00 | 24.03 |
| ATOM | 17475 | CG | ASN | 2606 | 45.751 | 18.816 | 65.384 | 1.00 | 27.25 |
| ATOM | 17476 | OD1 | ASN | 2606 | 45.935 | 19.737 | 66.182 | 1.00 | 30.24 |
| ATOM | 17477 | ND2 | ASN | 2606 | 46.513 | 17.729 | 65.360 | 1.00 | 21.63 |
| ATOM | 17478 | C | ASN | 2606 | 45.935 | 17.649 | 62.644 | 1.00 | 22.00 |
| ATOM | 17479 | O | ASN | 2606 | 46.428 | 18.680 | 62.185 | 1.00 | 22.27 |
| ATOM | 17480 | N | VAL | 2607 | 46.491 | 16.450 | 62.501 | 1.00 | 21.97 |
| ATOM | 17481 | CA | VAL | 2607 | 47.782 | 16.264 | 61.835 | 1.00 | 23.06 |
| ATOM | 17482 | CB | VAL | 2607 | 48.473 | 14.975 | 62.343 | 1.00 | 25.16 |
| ATOM | 17483 | CG1 | VAL | 2607 | 49.896 | 14.903 | 61.813 | 1.00 | 30.68 |
| ATOM | 17484 | CG2 | VAL | 2607 | 48.478 | 14.951 | 63.863 | 1.00 | 28.00 |
| ATOM | 17485 | C | VAL | 2607 | 47.698 | 16.210 | 60.308 | 1.00 | 22.15 |
| ATOM | 17486 | O | VAL | 2607 | 48.708 | 16.350 | 59.614 | 1.00 | 19.36 |
| ATOM | 17487 | N | THR | 2608 | 46.489 | 16.010 | 59.791 | 1.00 | 20.18 |
| ATOM | 17488 | CA | THR | 2608 | 46.273 | 15.945 | 58.354 | 1.00 | 18.55 |
| ATOM | 17489 | CB | THR | 2608 | 44.931 | 15.247 | 58.040 | 1.00 | 17.48 |
| ATOM | 17490 | OG1 | THR | 2608 | 43.864 | 15.942 | 58.698 | 1.00 | 17.94 |
| ATOM | 17491 | CG2 | THR | 2608 | 44.958 | 13.815 | 58.538 | 1.00 | 16.25 |
| ATOM | 17492 | C | THR | 2608 | 46.288 | 17.340 | 57.736 | 1.00 | 18.46 |
| ATOM | 17493 | O | THR | 2608 | 46.208 | 18.344 | 58.444 | 1.00 | 18.52 |
| ATOM | 17494 | N | ASP | 2609 | 46.392 | 17.392 | 56.414 | 1.00 | 18.90 |
| ATOM | 17495 | CA | ASP | 2609 | 46.438 | 18.653 | 55.685 | 1.00 | 19.61 |
| ATOM | 17496 | CB | ASP | 2609 | 46.858 | 18.384 | 54.238 | 1.00 | 20.80 |
| ATOM | 17497 | CG | ASP | 2609 | 48.209 | 17.698 | 54.143 | 1.00 | 22.85 |
| ATOM | 17498 | OD1 | ASP | 2609 | 49.214 | 18.315 | 54.550 | 1.00 | 25.80 |
| ATOM | 17499 | OD2 | ASP | 2609 | 48.269 | 16.544 | 53.662 | 1.00 | 25.46 |
| ATOM | 17500 | C | ASP | 2609 | 45.101 | 19.380 | 55.711 | 1.00 | 19.79 |
| ATOM | 17501 | O | ASP | 2609 | 45.051 | 20.607 | 55.693 | 1.00 | 18.76 |
| ATOM | 17502 | N | GLY | 2610 | 44.014 | 18.620 | 55.754 | 1.00 | 19.79 |
| ATOM | 17503 | CA | GLY | 2610 | 42.703 | 19.239 | 55.771 | 1.00 | 17.70 |
| ATOM | 17504 | C | GLY | 2610 | 41.726 | 18.492 | 56.646 | 1.00 | 14.64 |
| ATOM | 17505 | O | GLY | 2610 | 42.051 | 17.457 | 57.224 | 1.00 | 17.46 |
| ATOM | 17506 | N | GLN | 2611 | 40.516 | 19.024 | 56.752 | 1.00 | 17.50 |
| ATOM | 17507 | CA | GLN | 2611 | 39.482 | 18.398 | 57.559 | 1.00 | 17.89 |
| ATOM | 17508 | CB | GLN | 2611 | 39.268 | 19.190 | 58.853 | 1.00 | 19.92 |
| ATOM | 17509 | CG | GLN | 2611 | 40.465 | 19.210 | 59.799 | 1.00 | 20.42 |
| ATOM | 17510 | CD | GLN | 2611 | 40.848 | 17.829 | 60.291 | 1.00 | 22.00 |
| ATOM | 17511 | OE1 | GLN | 2611 | 39.984 | 17.015 | 60.627 | 1.00 | 24.29 |
| ATOM | 17512 | NE2 | GLN | 2611 | 42.148 | 17.561 | 60.354 | 1.00 | 19.83 |
| ATOM | 17513 | C | GLN | 2611 | 38.164 | 18.341 | 56.800 | 1.00 | 17.01 |
| ATOM | 17514 | O | GLN | 2611 | 37.911 | 19.151 | 55.905 | 1.00 | 17.06 |
| ATOM | 17515 | N | ILE | 2612 | 37.327 | 17.379 | 57.168 | 1.00 | 20.01 |
| ATOM | 17516 | CA | ILE | 2612 | 36.013 | 17.239 | 56.557 | 1.00 | 21.18 |
| ATOM | 17517 | CB | ILE | 2612 | 36.047 | 16.279 | 55.336 | 1.00 | 21.95 |
| ATOM | 17518 | CG2 | ILE | 2612 | 36.253 | 14.835 | 55.791 | 1.00 | 17.51 |
| ATOM | 17519 | CG1 | ILE | 2612 | 34.743 | 16.421 | 54.542 | 1.00 | 21.60 |
| ATOM | 17520 | CD1 | ILE | 2612 | 34.855 | 15.977 | 53.097 | 1.00 | 22.16 |
| ATOM | 17521 | C | ILE | 2612 | 35.043 | 16.724 | 57.611 | 1.00 | 23.20 |
| ATOM | 17522 | O | ILE | 2612 | 35.435 | 16.034 | 58.559 | 1.00 | 23.34 |
| ATOM | 17523 | N | LEU | 2613 | 33.774 | 17.072 | 57.458 | 1.00 | 25.17 |
| ATOM | 17524 | CA | LEU | 2613 | 32.774 | 16.644 | 58.416 | 1.00 | 27.65 |
| ATOM | 17525 | CB | LEU | 2613 | 32.932 | 17.476 | 59.686 | 1.00 | 31.09 |
| ATOM | 17526 | CG | LEU | 2613 | 32.465 | 16.923 | 61.030 | 1.00 | 34.00 |
| ATOM | 17527 | CD1 | LEU | 2613 | 32.834 | 17.930 | 62.111 | 1.00 | 34.54 |
| ATOM | 17528 | CD2 | LEU | 2613 | 30.962 | 16.673 | 61.020 | 1.00 | 35.29 |
| ATOM | 17529 | C | LEU | 2613 | 31.382 | 16.842 | 57.821 | 1.00 | 27.54 |
| ATOM | 17530 | O | LEU | 2613 | 31.168 | 17.767 | 57.044 | 1.00 | 26.77 |
| ATOM | 17531 | N | VAL | 2614 | 30.450 | 15.961 | 58.173 | 1.00 | 27.38 |
| ATOM | 17532 | CA | VAL | 2614 | 29.087 | 16.073 | 57.678 | 1.00 | 27.05 |
| ATOM | 17533 | CB | VAL | 2614 | 28.210 | 14.890 | 58.141 | 1.00 | 28.19 |
| ATOM | 17534 | CG1 | VAL | 2614 | 26.793 | 15.037 | 57.597 | 1.00 | 28.81 |
| ATOM | 17535 | CG2 | VAL | 2614 | 28.825 | 13.577 | 57.671 | 1.00 | 28.09 |
| ATOM | 17536 | C | VAL | 2614 | 28.523 | 17.375 | 58.238 | 1.00 | 25.84 |
| ATOM | 17537 | O | VAL | 2614 | 28.502 | 17.590 | 59.449 | 1.00 | 26.11 |
| ATOM | 17538 | N | MET | 2615 | 28.085 | 18.251 | 57.344 | 1.00 | 24.55 |
| ATOM | 17539 | CA | MET | 2615 | 27.541 | 19.532 | 57.753 | 1.00 | 22.58 |
| ATOM | 17540 | CB | MET | 2615 | 26.987 | 20.274 | 56.546 | 1.00 | 20.69 |
| ATOM | 17541 | CG | MET | 2615 | 25.813 | 19.564 | 55.890 | 1.00 | 18.65 |
| ATOM | 17542 | SD | MET | 2615 | 24.658 | 20.775 | 55.265 | 1.00 | 19.02 |
| ATOM | 17543 | CE | MET | 2615 | 23.584 | 20.962 | 56.690 | 1.00 | 18.27 |
| ATOM | 17544 | C | MET | 2615 | 26.440 | 19.393 | 58.806 | 1.00 | 22.84 |
| ATOM | 17545 | O | MET | 2615 | 26.255 | 20.281 | 59.632 | 1.00 | 22.93 |
| ATOM | 17546 | N | HIS | 2616 | 25.701 | 18.288 | 58.776 | 1.00 | 21.47 |
| ATOM | 17547 | CA | HIS | 2616 | 24.627 | 18.100 | 59.737 | 1.00 | 21.54 |
| ATOM | 17548 | CB | HIS | 2616 | 23.741 | 16.930 | 59.314 | 1.00 | 21.41 |
| ATOM | 17549 | CG | HIS | 2616 | 23.013 | 17.181 | 58.030 | 1.00 | 18.70 |
| ATOM | 17550 | CD2 | HIS | 2616 | 23.417 | 17.071 | 56.743 | 1.00 | 16.47 |
| ATOM | 17551 | ND1 | HIS | 2616 | 21.736 | 17.697 | 57.987 | 1.00 | 20.75 |
| ATOM | 17552 | CE1 | HIS | 2616 | 21.385 | 17.895 | 56.729 | 1.00 | 18.61 |
| ATOM | 17553 | NE2 | HIS | 2616 | 22.388 | 17.524 | 55.955 | 1.00 | 20.61 |
| ATOM | 17554 | C | HIS | 2616 | 25.122 | 17.916 | 61.161 | 1.00 | 23.40 |
| ATOM | 17555 | O | HIS | 2616 | 24.420 | 18.266 | 62.113 | 1.00 | 21.84 |
| ATOM | 17556 | N | ASP | 2617 | 26.328 | 17.379 | 61.322 | 1.00 | 24.60 |
| ATOM | 17557 | CA | ASP | 2617 | 26.880 | 17.188 | 62.664 | 1.00 | 28.05 |
| ATOM | 17558 | CB | ASP | 2617 | 27.918 | 16.060 | 62.677 | 1.00 | 29.84 |
| ATOM | 17559 | CG | ASP | 2617 | 27.311 | 14.703 | 62.396 | 1.00 | 32.71 |
| ATOM | 17560 | OD1 | ASP | 2617 | 26.339 | 14.322 | 63.088 | 1.00 | 30.70 |
| ATOM | 17561 | OD2 | ASP | 2617 | 27.818 | 14.012 | 61.487 | 1.00 | 34.71 |
| ATOM | 17562 | C | ASP | 2617 | 27.535 | 18.473 | 63.153 | 1.00 | 28.63 |
| ATOM | 17563 | O | ASP | 2617 | 27.681 | 18.679 | 64.356 | 1.00 | 30.14 |
| ATOM | 17564 | N | ALA | 2618 | 27.928 | 19.333 | 62.217 | 1.00 | 29.76 |
| ATOM | 17565 | CA | ALA | 2618 | 28.577 | 20.593 | 62.561 | 1.00 | 31.50 |
| ATOM | 17566 | CB | ALA | 2618 | 29.311 | 21.152 | 61.339 | 1.00 | 30.82 |
| ATOM | 17567 | C | ALA | 2618 | 27.606 | 21.635 | 63.116 | 1.00 | 32.49 |
| ATOM | 17568 | O | ALA | 2618 | 28.029 | 22.630 | 63.707 | 1.00 | 33.61 |
| ATOM | 17569 | N | PHE | 2619 | 26.308 | 21.411 | 62.931 | 1.00 | 33.51 |
| ATOM | 17570 | CA | PHE | 2619 | 25.311 | 22.350 | 63.436 | 1.00 | 33.26 |
| ATOM | 17571 | CB | PHE | 2619 | 24.524 | 22.962 | 62.280 | 1.00 | 35.56 |
| ATOM | 17572 | CG | PHE | 2619 | 25.394 | 23.485 | 61.174 | 1.00 | 37.64 |
| ATOM | 17573 | CD1 | PHE | 2619 | 26.457 | 24.336 | 61.452 | 1.00 | 39.02 |
| ATOM | 17574 | CD2 | PHE | 2619 | 25.156 | 23.119 | 59.852 | 1.00 | 38.68 |
| ATOM | 17575 | CE1 | PHE | 2619 | 27.277 | 24.814 | 60.432 | 1.00 | 39.78 |
| ATOM | 17576 | CE2 | PHE | 2619 | 25.969 | 23.591 | 58.821 | 1.00 | 39.59 |
| ATOM | 17577 | CZ | PHE | 2619 | 27.030 | 24.439 | 59.114 | 1.00 | 40.73 |
| ATOM | 17578 | C | PHE | 2619 | 24.359 | 21.694 | 64.423 | 1.00 | 32.67 |
| ATOM | 17579 | O | PHE | 2619 | 23.247 | 22.173 | 64.642 | 1.00 | 33.99 |
| ATOM | 17580 | N | GLY | 2620 | 24.806 | 20.596 | 65.023 | 1.00 | 33.38 |
| ATOM | 17581 | CA | GLY | 2620 | 23.989 | 19.889 | 65.996 | 1.00 | 32.34 |
| ATOM | 17582 | C | GLY | 2620 | 22.618 | 19.483 | 65.490 | 1.00 | 32.02 |
| ATOM | 17583 | O | GLY | 2620 | 21.715 | 19.214 | 66.283 | 1.00 | 32.91 |
| ATOM | 17584 | N | ILE | 2621 | 22.454 | 19.435 | 64.172 | 1.00 | 30.10 |
| ATOM | 17585 | CA | ILE | 2621 | 21.176 | 19.046 | 63.589 | 1.00 | 29.00 |
| ATOM | 17586 | CB | ILE | 2621 | 21.170 | 19.248 | 62.057 | 1.00 | 26.35 |
| ATOM | 17587 | CG2 | ILE | 2621 | 19.867 | 18.713 | 61.464 | 1.00 | 25.62 |
| ATOM | 17588 | CG1 | ILE | 2621 | 21.332 | 20.730 | 61.729 | 1.00 | 24.08 |
| ATOM | 17589 | CD1 | ILE | 2621 | 21.695 | 21.009 | 60.295 | 1.00 | 24.54 |
| ATOM | 17590 | C | ILE | 2621 | 20.900 | 17.581 | 63.895 | 1.00 | 30.31 |
| ATOM | 17591 | O | ILE | 2621 | 19.780 | 17.209 | 64.224 | 1.00 | 29.60 |
| ATOM | 17592 | N | THR | 2622 | 21.936 | 16.756 | 63.799 | 1.00 | 32.59 |
| ATOM | 17593 | CA | THR | 2622 | 21.794 | 15.329 | 64.053 | 1.00 | 35.67 |
| ATOM | 17594 | CB | THR | 2622 | 23.035 | 14.556 | 63.560 | 1.00 | 35.42 |
| ATOM | 17595 | OG1 | THR | 2622 | 24.141 | 14.813 | 64.434 | 1.00 | 36.19 |
| ATOM | 17596 | CG2 | THR | 2622 | 23.407 | 15.005 | 62.158 | 1.00 | 31.84 |
| ATOM | 17597 | C | THR | 2622 | 21.581 | 15.037 | 65.535 | 1.00 | 38.18 |
| ATOM | 17598 | O | THR | 2622 | 22.311 | 15.549 | 66.383 | 1.00 | 38.40 |
| ATOM | 17599 | N | GLY | 2623 | 20.571 | 14.220 | 65.827 | 1.00 | 41.10 |
| ATOM | 17600 | CA | GLY | 2623 | 20.252 | 13.844 | 67.194 | 1.00 | 46.07 |
| ATOM | 17601 | C | GLY | 2623 | 20.921 | 14.664 | 68.279 | 1.00 | 49.46 |
| ATOM | 17602 | O | GLY | 2623 | 20.755 | 15.884 | 68.342 | 1.00 | 50.21 |
| ATOM | 17603 | N | GLY | 2624 | 21.685 | 13.992 | 69.135 | 1.00 | 51.80 |
| ATOM | 17604 | CA | GLY | 2624 | 22.370 | 14.681 | 70.213 | 1.00 | 53.86 |
| ATOM | 17605 | C | GLY | 2624 | 23.726 | 14.086 | 70.538 | 1.00 | 55.07 |
| ATOM | 17606 | O | GLY | 2624 | 24.670 | 14.811 | 70.856 | 1.00 | 56.17 |
| ATOM | 17607 | N | HIS | 2625 | 23.829 | 12.763 | 70.461 | 1.00 | 55.75 |
| ATOM | 17608 | CA | HIS | 2625 | 25.088 | 12.087 | 70.754 | 1.00 | 56.39 |
| ATOM | 17609 | CB | HIS | 2625 | 24.817 | 10.702 | 71.352 | 1.00 | 58.60 |
| ATOM | 17610 | CG | HIS | 2625 | 24.122 | 10.744 | 72.678 | 1.00 | 61.61 |
| ATOM | 17611 | CD2 | HIS | 2625 | 22.939 | 10.223 | 73.083 | 1.00 | 62.23 |
| ATOM | 17612 | ND1 | HIS | 2625 | 24.651 | 11.389 | 73.776 | 1.00 | 62.65 |
| ATOM | 17613 | CE1 | HIS | 2625 | 23.825 | 11.264 | 74.799 | 1.00 | 62.69 |
| ATOM | 17614 | NE2 | HIS | 2625 | 22.778 | 10.561 | 74.406 | 1.00 | 63.05 |
| ATOM | 17615 | C | HIS | 2625 | 25.957 | 11.959 | 69.504 | 1.00 | 55.11 |
| ATOM | 17616 | O | HIS | 2625 | 26.250 | 10.852 | 69.053 | 1.00 | 55.29 |
| ATOM | 17617 | N | ILE | 2626 | 26.368 | 13.099 | 68.953 | 1.00 | 53.22 |
| ATOM | 17618 | CA | ILE | 2626 | 27.207 | 13.123 | 67.756 | 1.00 | 50.84 |
| ATOM | 17619 | CB | ILE | 2626 | 27.596 | 14.566 | 67.365 | 1.00 | 51.18 |
| ATOM | 17620 | CG2 | ILE | 2626 | 26.352 | 15.369 | 67.024 | 1.00 | 52.29 |
| ATOM | 17621 | CG1 | ILE | 2626 | 28.377 | 15.218 | 68.508 | 1.00 | 50.93 |
| ATOM | 17622 | CD1 | ILE | 2626 | 29.050 | 16.524 | 68.131 | 1.00 | 51.04 |
| ATOM | 17623 | C | ILE | 2626 | 28.494 | 12.335 | 67.984 | 1.00 | 47.86 |
| ATOM | 17624 | O | ILE | 2626 | 28.875 | 12.067 | 69.122 | 1.00 | 47.90 |
| ATOM | 17625 | N | PRO | 2627 | 29.189 | 11.963 | 66.899 | 1.00 | 45.93 |
| ATOM | 17626 | CD | PRO | 2627 | 28.901 | 12.266 | 65.485 | 1.00 | 45.13 |
| ATOM | 17627 | CA | PRO | 2627 | 30.436 | 11.203 | 67.026 | 1.00 | 43.64 |
| ATOM | 17628 | CB | PRO | 2627 | 30.801 | 10.904 | 65.574 | 1.00 | 43.90 |
| ATOM | 17629 | CG | PRO | 2627 | 30.254 | 12.089 | 64.843 | 1.00 | 45.31 |
| ATOM | 17630 | C | PRO | 2627 | 31.533 | 11.973 | 67.762 | 1.00 | 41.87 |
| ATOM | 17631 | O | PRO | 2627 | 31.599 | 13.198 | 67.687 | 1.00 | 41.53 |
| ATOM | 17632 | N | LYS | 2628 | 32.390 | 11.243 | 68.472 | 1.00 | 40.14 |
| ATOM | 17633 | CA | LYS | 2628 | 33.486 | 11.846 | 69.224 | 1.00 | 39.09 |
| ATOM | 17634 | CB | LYS | 2628 | 34.362 | 10.759 | 69.864 | 1.00 | 41.61 |
| ATOM | 17635 | CG | LYS | 2628 | 34.081 | 10.500 | 71.340 | 1.00 | 44.14 |
| ATOM | 17636 | CD | LYS | 2628 | 32.661 | 9.998 | 71.567 | 1.00 | 46.92 |
| ATOM | 17637 | CE | LYS | 2628 | 32.359 | 9.839 | 73.051 | 1.00 | 47.79 |
| ATOM | 17638 | NZ | LYS | 2628 | 30.956 | 9.403 | 73.304 | 1.00 | 49.12 |
| ATOM | 17639 | C | LYS | 2628 | 34.372 | 12.748 | 68.376 | 1.00 | 36.45 |
| ATOM | 17640 | O | LYS | 2628 | 34.905 | 13.743 | 68.865 | 1.00 | 36.12 |
| ATOM | 17641 | N | PHE | 2629 | 34.531 | 12.405 | 67.103 | 1.00 | 32.52 |
| ATOM | 17642 | CA | PHE | 2629 | 35.387 | 13.189 | 66.224 | 1.00 | 29.15 |
| ATOM | 17643 | CB | PHE | 2629 | 35.876 | 12.319 | 65.057 | 1.00 | 29.64 |
| ATOM | 17644 | CG | PHE | 2629 | 34.772 | 11.753 | 64.216 | 1.00 | 29.08 |
| ATOM | 17645 | CD1 | PHE | 2629 | 34.068 | 12.561 | 63.328 | 1.00 | 29.61 |
| ATOM | 17646 | CD2 | PHE | 2629 | 34.431 | 10.411 | 64.318 | 1.00 | 30.24 |
| ATOM | 17647 | CE1 | PHE | 2629 | 33.035 | 12.036 | 62.550 | 1.00 | 32.08 |
| ATOM | 17648 | CE2 | PHE | 2629 | 33.403 | 9.873 | 63.550 | 1.00 | 32.17 |
| ATOM | 17649 | CZ | PHE | 2629 | 32.702 | 10.688 | 62.661 | 1.00 | 31.61 |
| ATOM | 17650 | C | PHE | 2629 | 34.742 | 14.458 | 65.692 | 1.00 | 26.59 |
| ATOM | 17651 | O | PHE | 2629 | 35.426 | 15.316 | 65.135 | 1.00 | 24.60 |
| ATOM | 17652 | N | ALA | 2630 | 33.432 | 14.587 | 65.869 | 1.00 | 24.68 |
| ATOM | 17653 | CA | ALA | 2630 | 32.720 | 15.764 | 65.388 | 1.00 | 24.35 |
| ATOM | 17654 | CB | ALA | 2630 | 31.342 | 15.359 | 64.856 | 1.00 | 24.75 |
| ATOM | 17655 | C | ALA | 2630 | 32.559 | 16.822 | 66.473 | 1.00 | 23.16 |
| ATOM | 17656 | O | ALA | 2630 | 32.719 | 16.539 | 67.659 | 1.00 | 23.58 |
| ATOM | 17657 | N | LYS | 2631 | 32.252 | 18.043 | 66.046 | 1.00 | 23.38 |
| ATOM | 17658 | CA | LYS | 2631 | 32.039 | 19.164 | 66.955 | 1.00 | 23.65 |
| ATOM | 17659 | CB | LYS | 2631 | 33.319 | 19.993 | 67.105 | 1.00 | 23.84 |
| ATOM | 17660 | CG | LYS | 2631 | 33.127 | 21.250 | 67.947 | 1.00 | 25.08 |
| ATOM | 17661 | CD | LYS | 2631 | 34.435 | 21.990 | 68.159 | 1.00 | 25.96 |
| ATOM | 17662 | CE | LYS | 2631 | 34.227 | 23.261 | 68.968 | 1.00 | 28.22 |
| ATOM | 17663 | NZ | LYS | 2631 | 35.499 | 24.020 | 69.116 | 1.00 | 29.85 |
| ATOM | 17664 | C | LYS | 2631 | 30.921 | 20.068 | 66.444 | 1.00 | 22.92 |
| ATOM | 17665 | O | LYS | 2631 | 30.885 | 20.422 | 65.263 | 1.00 | 21.69 |
| ATOM | 17666 | N | ASN | 2632 | 30.015 | 20.436 | 67.345 | 1.00 | 23.42 |
| ATOM | 17667 | CA | ASN | 2632 | 28.889 | 21.310 | 67.026 | 1.00 | 22.87 |
| ATOM | 17668 | CB | ASN | 2632 | 27.771 | 21.121 | 68.058 | 1.00 | 22.06 |
| ATOM | 17669 | CG | ASN | 2632 | 26.566 | 22.005 | 67.792 | 1.00 | 22.77 |
| ATOM | 17670 | OD1 | ASN | 2632 | 26.626 | 22.943 | 66.994 | 1.00 | 23.52 |
| ATOM | 17671 | ND2 | ASN | 2632 | 25.463 | 21.716 | 68.475 | 1.00 | 20.23 |
| ATOM | 17672 | C | ASN | 2632 | 29.384 | 22.748 | 67.080 | 1.00 | 22.32 |
| ATOM | 17673 | O | ASN | 2632 | 29.559 | 23.306 | 68.167 | 1.00 | 22.04 |
| ATOM | 17674 | N | PHE | 2633 | 29.617 | 23.348 | 65.918 | 1.00 | 20.88 |
| ATOM | 17675 | CA | PHE | 2633 | 30.103 | 24.724 | 65.871 | 1.00 | 22.92 |
| ATOM | 17676 | CB | PHE | 2633 | 30.879 | 24.987 | 64.576 | 1.00 | 23.76 |
| ATOM | 17677 | CG | PHE | 2633 | 32.182 | 24.245 | 64.492 | 1.00 | 25.62 |
| ATOM | 17678 | CD1 | PHE | 2633 | 32.224 | 22.938 | 64.020 | 1.00 | 25.78 |
| ATOM | 17679 | CD2 | PHE | 2633 | 33.363 | 24.837 | 64.933 | 1.00 | 25.99 |
| ATOM | 17680 | CE1 | PHE | 2633 | 33.421 | 22.229 | 63.988 | 1.00 | 25.39 |
| ATOM | 17681 | CE2 | PHE | 2633 | 34.565 | 24.138 | 64.908 | 1.00 | 24.67 |
| ATOM | 17682 | CZ | PHE | 2633 | 34.593 | 22.831 | 64.435 | 1.00 | 27.61 |
| ATOM | 17683 | C | PHE | 2633 | 29.002 | 25.762 | 66.021 | 1.00 | 23.51 |
| ATOM | 17684 | O | PHE | 2633 | 29.287 | 26.940 | 66.230 | 1.00 | 22.07 |
| ATOM | 17685 | N | LEU | 2634 | 27.748 | 25.329 | 65.917 | 1.00 | 23.90 |
| ATOM | 17686 | CA | LEU | 2634 | 26.625 | 26.246 | 66.056 | 1.00 | 25.40 |
| ATOM | 17687 | CB | LEU | 2634 | 25.351 | 25.628 | 65.485 | 1.00 | 23.46 |
| ATOM | 17688 | CG | LEU | 2634 | 24.073 | 26.438 | 65.713 | 1.00 | 19.60 |
| ATOM | 17689 | CD1 | LEU | 2634 | 24.167 | 27.786 | 65.009 | 1.00 | 19.80 |
| ATOM | 17690 | CD2 | LEU | 2634 | 22.876 | 25.651 | 65.185 | 1.00 | 17.49 |
| ATOM | 17691 | C | LEU | 2634 | 26.397 | 26.597 | 67.521 | 1.00 | 28.16 |
| ATOM | 17692 | O | LEU | 2634 | 26.108 | 27.748 | 67.853 | 1.00 | 27.99 |
| ATOM | 17693 | N | ALA | 2635 | 26.516 | 25.592 | 68.384 | 1.00 | 30.92 |
| ATOM | 17694 | CA | ALA | 2635 | 26.326 | 25.760 | 69.820 | 1.00 | 36.08 |
| ATOM | 17695 | CB | ALA | 2635 | 26.545 | 24.434 | 70.529 | 1.00 | 35.24 |
| ATOM | 17696 | C | ALA | 2635 | 27.297 | 26.801 | 70.356 | 1.00 | 40.21 |
| ATOM | 17697 | O | ALA | 2635 | 26.965 | 27.569 | 71.260 | 1.00 | 41.94 |
| ATOM | 17698 | N | GLU | 2636 | 28.498 | 26.812 | 69.786 | 1.00 | 43.52 |
| ATOM | 17699 | CA | GLU | 2636 | 29.550 | 27.748 | 70.170 | 1.00 | 47.26 |
| ATOM | 17700 | CB | GLU | 2636 | 30.885 | 27.313 | 69.550 | 1.00 | 48.66 |
| ATOM | 17701 | CG | GLU | 2636 | 31.220 | 25.832 | 69.730 | 1.00 | 52.15 |
| ATOM | 17702 | CD | GLU | 2636 | 31.594 | 25.471 | 71.158 | 1.00 | 53.80 |
| ATOM | 17703 | OE1 | GLU | 2636 | 30.830 | 25.815 | 72.085 | 1.00 | 55.93 |
| ATOM | 17704 | OE2 | GLU | 2636 | 32.652 | 24.834 | 71.354 | 1.00 | 54.50 |
| ATOM | 17705 | C | GLU | 2636 | 29.183 | 29.147 | 69.670 | 1.00 | 47.91 |
| ATOM | 17706 | O | GLU | 2636 | 30.017 | 30.052 | 69.645 | 1.00 | 48.49 |
| ATOM | 17707 | N | THR | 2637 | 27.927 | 29.306 | 69.263 | 1.00 | 48.71 |
| ATOM | 17708 | CA | THR | 2637 | 27.418 | 30.575 | 68.764 | 1.00 | 48.27 |
| ATOM | 17709 | CB | THR | 2637 | 28.031 | 30.914 | 67.384 | 1.00 | 48.89 |
| ATOM | 17710 | OG1 | THR | 2637 | 27.518 | 32.171 | 66.927 | 1.00 | 48.26 |
| ATOM | 17711 | CG2 | THR | 2637 | 27.693 | 29.833 | 66.365 | 1.00 | 49.85 |
| ATOM | 17712 | C | THR | 2637 | 25.894 | 30.501 | 68.636 | 1.00 | 47.68 |
| ATOM | 17713 | O | THR | 2637 | 25.236 | 29.744 | 69.356 | 1.00 | 48.21 |
| ATOM | 17714 | N | GLY | 2638 | 25.336 | 31.289 | 67.724 | 1.00 | 45.54 |
| ATOM | 17715 | CA | GLY | 2638 | 23.898 | 31.283 | 67.526 | 1.00 | 43.10 |
| ATOM | 17716 | C | GLY | 2638 | 23.555 | 31.571 | 66.082 | 1.00 | 40.35 |
| ATOM | 17717 | O | GLY | 2638 | 22.403 | 31.851 | 65.749 | 1.00 | 40.49 |
| ATOM | 17718 | N | ASP | 2639 | 24.567 | 31.487 | 65.224 | 1.00 | 37.74 |
| ATOM | 17719 | CA | ASP | 2639 | 24.411 | 31.755 | 63.799 | 1.00 | 35.76 |
| ATOM | 17720 | CB | ASP | 2639 | 25.025 | 33.116 | 63.469 | 1.00 | 39.24 |
| ATOM | 17721 | CG | ASP | 2639 | 24.688 | 33.573 | 62.078 | 1.00 | 41.90 |
| ATOM | 17722 | OD1 | ASP | 2639 | 25.570 | 34.144 | 61.405 | 1.00 | 42.76 |
| ATOM | 17723 | OD2 | ASP | 2639 | 23.526 | 33.367 | 61.662 | 1.00 | 46.27 |
| ATOM | 17724 | C | ASP | 2639 | 25.110 | 30.676 | 62.967 | 1.00 | 32.25 |
| ATOM | 17725 | O | ASP | 2639 | 26.276 | 30.361 | 63.206 | 1.00 | 30.44 |
| ATOM | 17726 | N | ILE | 2640 | 24.409 | 30.119 | 61.985 | 1.00 | 28.72 |
| ATOM | 17727 | CA | ILE | 2640 | 25.005 | 29.083 | 61.149 | 1.00 | 25.76 |
| ATOM | 17728 | CB | ILE | 2640 | 23.959 | 28.470 | 60.176 | 1.00 | 25.25 |
| ATOM | 17729 | CG2 | ILE | 2640 | 24.657 | 27.628 | 59.111 | 1.00 | 23.48 |
| ATOM | 17730 | CG1 | ILE | 2640 | 22.952 | 27.638 | 60.969 | 1.00 | 22.50 |
| ATOM | 17731 | CD1 | ILE | 2640 | 21.751 | 27.150 | 60.161 | 1.00 | 23.27 |
| ATOM | 17732 | C | ILE | 2640 | 26.204 | 29.612 | 60.358 | 1.00 | 25.06 |
| ATOM | 17733 | O | ILE | 2640 | 27.251 | 28.963 | 60.299 | 1.00 | 25.46 |
| ATOM | 17734 | N | ARG | 2641 | 26.061 | 30.785 | 59.752 | 1.00 | 24.74 |
| ATOM | 17735 | CA | ARG | 2641 | 27.169 | 31.356 | 58.993 | 1.00 | 23.19 |
| ATOM | 17736 | CB | ARG | 2641 | 26.739 | 32.642 | 58.285 | 1.00 | 22.91 |
| ATOM | 17737 | CG | ARG | 2641 | 25.858 | 32.392 | 57.077 | 1.00 | 25.25 |
| ATOM | 17738 | CD | ARG | 2641 | 25.407 | 33.687 | 56.433 | 1.00 | 24.40 |
| ATOM | 17739 | NE | ARG | 2641 | 24.650 | 33.441 | 55.210 | 1.00 | 27.34 |
| ATOM | 17740 | CZ | ARG | 2641 | 23.453 | 32.863 | 55.166 | 1.00 | 26.81 |
| ATOM | 17741 | NH1 | ARG | 2641 | 22.858 | 32.468 | 56.283 | 1.00 | 25.30 |
| ATOM | 17742 | NH2 | ARG | 2641 | 22.854 | 32.674 | 53.996 | 1.00 | 28.38 |
| ATOM | 17743 | C | ARG | 2641 | 28.348 | 31.635 | 59.917 | 1.00 | 23.31 |
| ATOM | 17744 | O | ARG | 2641 | 29.508 | 31.524 | 59.511 | 1.00 | 20.74 |
| ATOM | 17745 | N | ALA | 2642 | 28.047 | 31.992 | 61.162 | 1.00 | 23.35 |
| ATOM | 17746 | CA | ALA | 2642 | 29.089 | 32.253 | 62.143 | 1.00 | 25.68 |
| ATOM | 17747 | CB | ALA | 2642 | 28.484 | 32.881 | 63.404 | 1.00 | 26.54 |
| ATOM | 17748 | C | ALA | 2642 | 29.765 | 30.929 | 62.484 | 1.00 | 26.20 |
| ATOM | 17749 | O | ALA | 2642 | 30.979 | 30.874 | 62.687 | 1.00 | 26.09 |
| ATOM | 17750 | N | ALA | 2643 | 28.972 | 29.861 | 62.538 | 1.00 | 24.61 |
| ATOM | 17751 | CA | ALA | 2643 | 29.504 | 28.538 | 62.847 | 1.00 | 24.08 |
| ATOM | 17752 | CB | ALA | 2643 | 28.360 | 27.535 | 62.989 | 1.00 | 25.00 |
| ATOM | 17753 | C | ALA | 2643 | 30.460 | 28.098 | 61.738 | 1.00 | 23.31 |
| ATOM | 17754 | O | ALA | 2643 | 31.491 | 27.492 | 62.008 | 1.00 | 22.53 |
| ATOM | 17755 | N | VAL | 2644 | 30.107 | 28.413 | 60.494 | 1.00 | 23.63 |
| ATOM | 17756 | CA | VAL | 2644 | 30.932 | 28.066 | 59.340 | 1.00 | 23.56 |
| ATOM | 17757 | CB | VAL | 2644 | 30.262 | 28.501 | 58.019 | 1.00 | 23.61 |
| ATOM | 17758 | CG1 | VAL | 2644 | 31.193 | 28.248 | 56.850 | 1.00 | 23.86 |
| ATOM | 17759 | CG2 | VAL | 2644 | 28.953 | 27.737 | 57.817 | 1.00 | 24.12 |
| ATOM | 17760 | C | VAL | 2644 | 32.287 | 28.762 | 59.443 | 1.00 | 25.21 |
| ATOM | 17761 | O | VAL | 2644 | 33.332 | 28.135 | 59.290 | 1.00 | 23.86 |
| ATOM | 17762 | N | ARG | 2645 | 32.257 | 30.066 | 59.689 | 1.00 | 24.95 |
| ATOM | 17763 | CA | ARG | 2645 | 33.478 | 30.846 | 59.828 | 1.00 | 27.28 |
| ATOM | 17764 | CB | ARG | 2645 | 33.136 | 32.318 | 60.084 | 1.00 | 27.82 |
| ATOM | 17765 | CG | ARG | 2645 | 32.446 | 33.015 | 58.922 | 1.00 | 28.30 |
| ATOM | 17766 | CD | ARG | 2645 | 32.294 | 34.509 | 59.187 | 1.00 | 32.06 |
| ATOM | 17767 | NE | ARG | 2645 | 31.361 | 34.790 | 60.279 | 1.00 | 34.28 |
| ATOM | 17768 | CZ | ARG | 2645 | 30.060 | 35.018 | 60.120 | 1.00 | 36.23 |
| ATOM | 17769 | NH1 | ARG | 2645 | 29.522 | 35.002 | 58.906 | 1.00 | 34.78 |
| ATOM | 17770 | NH2 | ARG | 2645 | 29.297 | 35.265 | 61.177 | 1.00 | 37.25 |
| ATOM | 17771 | C | ARG | 2645 | 34.340 | 30.303 | 60.970 | 1.00 | 27.28 |
| ATOM | 17772 | O | ARG | 2645 | 35.561 | 30.227 | 60.849 | 1.00 | 29.76 |
| ATOM | 17773 | N | GLN | 2646 | 33.702 | 29.918 | 62.072 | 1.00 | 27.84 |
| ATOM | 17774 | CA | GLN | 2646 | 34.411 | 29.384 | 63.233 | 1.00 | 27.15 |
| ATOM | 17775 | CB | GLN | 2646 | 33.428 | 29.144 | 64.384 | 1.00 | 31.32 |
| ATOM | 17776 | CG | GLN | 2646 | 34.082 | 28.858 | 65.726 | 1.00 | 36.87 |
| ATOM | 17777 | CD | GLN | 2646 | 33.092 | 28.337 | 66.757 | 1.00 | 41.60 |
| ATOM | 17778 | OE1 | GLN | 2646 | 31.974 | 28.849 | 66.878 | 1.00 | 45.33 |
| ATOM | 17779 | NE2 | GLN | 2646 | 33.503 | 27.319 | 67.512 | 1.00 | 43.04 |
| ATOM | 17780 | C | GLN | 2646 | 35.108 | 28.074 | 62.879 | 1.00 | 27.46 |
| ATOM | 17781 | O | GLN | 2646 | 36.251 | 27.835 | 63.275 | 1.00 | 24.55 |
| ATOM | 17782 | N | TYR | 2647 | 34.406 | 27.222 | 62.135 | 1.00 | 24.93 |
| ATOM | 17783 | CA | TYR | 2647 | 34.946 | 25.936 | 61.719 | 1.00 | 23.55 |
| ATOM | 17784 | CB | TYR | 2647 | 33.881 | 25.159 | 60.934 | 1.00 | 22.15 |
| ATOM | 17785 | CG | TYR | 2647 | 34.399 | 23.949 | 60.194 | 1.00 | 20.81 |
| ATOM | 17786 | CD1 | TYR | 2647 | 35.147 | 22.972 | 60.848 | 1.00 | 22.12 |
| ATOM | 17787 | CE1 | TYR | 2647 | 35.599 | 21.833 | 60.171 | 1.00 | 22.90 |
| ATOM | 17788 | CD2 | TYR | 2647 | 34.113 | 23.764 | 58.846 | 1.00 | 21.36 |
| ATOM | 17789 | CE2 | TYR | 2647 | 34.555 | 22.632 | 58.160 | 1.00 | 21.37 |
| ATOM | 17790 | CZ | TYR | 2647 | 35.296 | 21.675 | 58.827 | 1.00 | 23.37 |
| ATOM | 17791 | OH | TYR | 2647 | 35.742 | 20.558 | 58.149 | 1.00 | 23.07 |
| ATOM | 17792 | C | TYR | 2647 | 36.195 | 26.131 | 60.861 | 1.00 | 23.03 |
| ATOM | 17793 | O | TYR | 2647 | 37.210 | 25.465 | 61.064 | 1.00 | 22.24 |
| ATOM | 17794 | N | MET | 2648 | 36.112 | 27.053 | 59.909 | 1.00 | 23.54 |
| ATOM | 17795 | CA | MET | 2648 | 37.233 | 27.341 | 59.023 | 1.00 | 24.40 |
| ATOM | 17796 | CB | MET | 2648 | 36.817 | 28.368 | 57.968 | 1.00 | 24.61 |
| ATOM | 17797 | CG | MET | 2648 | 35.748 | 27.868 | 57.000 | 1.00 | 25.72 |
| ATOM | 17798 | SD | MET | 2648 | 35.032 | 29.213 | 56.049 | 1.00 | 29.67 |
| ATOM | 17799 | CE | MET | 2648 | 36.345 | 29.525 | 54.905 | 1.00 | 29.14 |
| ATOM | 17800 | C | MET | 2648 | 38.420 | 27.870 | 59.816 | 1.00 | 25.45 |
| ATOM | 17801 | O | MET | 2648 | 39.573 | 27.589 | 59.490 | 1.00 | 25.58 |
| ATOM | 17802 | N | ALA | 2649 | 38.133 | 28.636 | 60.861 | 1.00 | 25.72 |
| ATOM | 17803 | CA | ALA | 2649 | 39.188 | 29.208 | 61.690 | 1.00 | 25.41 |
| ATOM | 17804 | CB | ALA | 2649 | 38.609 | 30.311 | 62.575 | 1.00 | 23.43 |
| ATOM | 17805 | C | ALA | 2649 | 39.901 | 28.165 | 62.552 | 1.00 | 25.02 |
| ATOM | 17806 | O | ALA | 2649 | 41.131 | 28.091 | 62.557 | 1.00 | 25.14 |
| ATOM | 17807 | N | GLU | 2650 | 39.133 | 27.361 | 63.280 | 1.00 | 25.28 |
| ATOM | 17808 | CA | GLU | 2650 | 39.715 | 26.346 | 64.152 | 1.00 | 26.69 |
| ATOM | 17809 | CB | GLU | 2650 | 38.622 | 25.662 | 64.981 | 1.00 | 28.05 |
| ATOM | 17810 | CG | GLU | 2650 | 38.206 | 26.465 | 66.208 | 1.00 | 29.62 |
| ATOM | 17811 | CD | GLU | 2650 | 37.113 | 25.799 | 67.014 | 1.00 | 29.84 |
| ATOM | 17812 | OE1 | GLU | 2650 | 37.152 | 24.562 | 67.173 | 1.00 | 32.30 |
| ATOM | 17813 | OE2 | GLU | 2650 | 36.221 | 26.518 | 67.506 | 1.00 | 33.86 |
| ATOM | 17814 | C | GLU | 2650 | 40.539 | 25.296 | 63.418 | 1.00 | 26.90 |
| ATOM | 17815 | O | GLU | 2650 | 41.482 | 24.738 | 63.980 | 1.00 | 27.32 |
| ATOM | 17816 | N | VAL | 2651 | 40.181 | 25.021 | 62.168 | 1.00 | 26.19 |
| ATOM | 17817 | CA | VAL | 2651 | 40.914 | 24.042 | 61.380 | 1.00 | 25.27 |
| ATOM | 17818 | CB | VAL | 2651 | 40.163 | 23.681 | 60.078 | 1.00 | 24.96 |
| ATOM | 17819 | CG1 | VAL | 2651 | 41.063 | 22.849 | 59.179 | 1.00 | 23.48 |
| ATOM | 17820 | CG2 | VAL | 2651 | 38.891 | 22.900 | 60.411 | 1.00 | 23.49 |
| ATOM | 17821 | C | VAL | 2651 | 42.296 | 24.576 | 61.010 | 1.00 | 25.94 |
| ATOM | 17822 | O | VAL | 2651 | 43.279 | 23.836 | 61.032 | 1.00 | 25.81 |
| ATOM | 17823 | N | GLU | 2652 | 42.362 | 25.863 | 60.683 | 1.00 | 27.40 |
| ATOM | 17824 | CA | GLU | 2652 | 43.616 | 26.493 | 60.293 | 1.00 | 30.89 |
| ATOM | 17825 | CB | GLU | 2652 | 43.345 | 27.865 | 59.665 | 1.00 | 31.68 |
| ATOM | 17826 | CG | GLU | 2652 | 44.595 | 28.556 | 59.129 | 1.00 | 36.32 |
| ATOM | 17827 | CD | GLU | 2652 | 44.282 | 29.686 | 58.159 | 1.00 | 39.22 |
| ATOM | 17828 | OE1 | GLU | 2652 | 45.229 | 30.374 | 57.715 | 1.00 | 40.91 |
| ATOM | 17829 | OE2 | GLU | 2652 | 43.091 | 29.885 | 57.836 | 1.00 | 40.55 |
| ATOM | 17830 | C | GLU | 2652 | 44.614 | 26.638 | 61.438 | 1.00 | 32.09 |
| ATOM | 17831 | O | GLU | 2652 | 45.791 | 26.305 | 61.290 | 1.00 | 32.01 |
| ATOM | 17832 | N | SER | 2653 | 44.146 | 27.125 | 62.582 | 1.00 | 34.17 |
| ATOM | 17833 | CA | SER | 2653 | 45.026 | 27.310 | 63.727 | 1.00 | 36.02 |
| ATOM | 17834 | CB | SER | 2653 | 44.427 | 28.338 | 64.687 | 1.00 | 39.04 |
| ATOM | 17835 | OG | SER | 2653 | 44.357 | 29.613 | 64.066 | 1.00 | 44.62 |
| ATOM | 17836 | C | SER | 2653 | 45.317 | 26.001 | 64.454 | 1.00 | 35.06 |
| ATOM | 17837 | O | SER | 2653 | 46.243 | 25.923 | 65.262 | 1.00 | 36.58 |
| ATOM | 17838 | N | GLY | 2654 | 44.528 | 24.971 | 64.165 | 1.00 | 33.11 |
| ATOM | 17839 | CA | GLY | 2654 | 44.754 | 23.679 | 64.789 | 1.00 | 29.91 |
| ATOM | 17840 | C | GLY | 2654 | 43.918 | 23.352 | 66.012 | 1.00 | 27.78 |
| ATOM | 17841 | O | GLY | 2654 | 44.111 | 22.302 | 66.625 | 1.00 | 30.35 |
| ATOM | 17842 | N | VAL | 2655 | 42.999 | 24.239 | 66.376 | 1.00 | 26.05 |
| ATOM | 17843 | CA | VAL | 2655 | 42.129 | 24.020 | 67.529 | 1.00 | 24.30 |
| ATOM | 17844 | CB | VAL | 2655 | 41.290 | 25.277 | 67.832 | 1.00 | 25.65 |
| ATOM | 17845 | CG1 | VAL | 2655 | 40.191 | 24.946 | 68.833 | 1.00 | 27.96 |
| ATOM | 17846 | CG2 | VAL | 2655 | 42.196 | 26.371 | 68.399 | 1.00 | 26.98 |
| ATOM | 17847 | C | VAL | 2655 | 41.190 | 22.839 | 67.288 | 1.00 | 23.05 |
| ATOM | 17848 | O | VAL | 2655 | 40.821 | 22.125 | 68.221 | 1.00 | 19.84 |
| ATOM | 17849 | N | TYR | 2656 | 40.807 | 22.644 | 66.030 | 1.00 | 23.31 |
| ATOM | 17850 | CA | TYR | 2656 | 39.926 | 21.541 | 65.659 | 1.00 | 23.29 |
| ATOM | 17851 | CB | TYR | 2656 | 38.554 | 22.054 | 65.203 | 1.00 | 24.58 |
| ATOM | 17852 | CG | TYR | 2656 | 37.625 | 20.927 | 64.795 | 1.00 | 26.25 |
| ATOM | 17853 | CD1 | TYR | 2656 | 37.053 | 20.092 | 65.755 | 1.00 | 26.34 |
| ATOM | 17854 | CE1 | TYR | 2656 | 36.272 | 19.000 | 65.389 | 1.00 | 27.58 |
| ATOM | 17855 | CD2 | TYR | 2656 | 37.384 | 20.646 | 63.450 | 1.00 | 26.60 |
| ATOM | 17856 | CE2 | TYR | 2656 | 36.602 | 19.553 | 63.069 | 1.00 | 26.50 |
| ATOM | 17857 | CZ | TYR | 2656 | 36.052 | 18.734 | 64.048 | 1.00 | 28.77 |
| ATOM | 17858 | OH | TYR | 2656 | 35.298 | 17.633 | 63.693 | 1.00 | 29.76 |
| ATOM | 17859 | C | TYR | 2656 | 40.539 | 20.739 | 64.520 | 1.00 | 22.07 |
| ATOM | 17860 | O | TYR | 2656 | 40.971 | 21.308 | 63.519 | 1.00 | 21.40 |
| ATOM | 17861 | N | PRO | 2657 | 40.575 | 19.407 | 64.659 | 1.00 | 23.11 |
| ATOM | 17862 | CD | PRO | 2657 | 40.839 | 18.474 | 63.551 | 1.00 | 25.30 |
| ATOM | 17863 | CA | PRO | 2657 | 40.064 | 18.682 | 65.827 | 1.00 | 25.15 |
| ATOM | 17864 | CB | PRO | 2657 | 39.863 | 17.266 | 65.293 | 1.00 | 25.49 |
| ATOM | 17865 | CG | PRO | 2657 | 40.930 | 17.153 | 64.267 | 1.00 | 26.67 |
| ATOM | 17866 | C | PRO | 2657 | 40.987 | 18.704 | 67.047 | 1.00 | 26.33 |
| ATOM | 17867 | O | PRO | 2657 | 42.194 | 18.937 | 66.934 | 1.00 | 24.35 |
| ATOM | 17868 | N | GLY | 2658 | 40.402 | 18.451 | 68.212 | 1.00 | 28.19 |
| ATOM | 17869 | CA | GLY | 2658 | 41.175 | 18.424 | 69.435 | 1.00 | 29.27 |
| ATOM | 17870 | C | GLY | 2658 | 41.405 | 16.990 | 69.870 | 1.00 | 31.45 |
| ATOM | 17871 | O | GLY | 2658 | 41.011 | 16.048 | 69.175 | 1.00 | 29.26 |
| ATOM | 17872 | N | GLU | 2659 | 42.039 | 16.825 | 71.025 | 1.00 | 31.72 |
| ATOM | 17873 | CA | GLU | 2659 | 42.328 | 15.506 | 71.568 | 1.00 | 33.60 |
| ATOM | 17874 | CB | GLU | 2659 | 43.166 | 15.638 | 72.845 | 1.00 | 35.63 |
| ATOM | 17875 | CG | GLU | 2659 | 43.629 | 14.310 | 73.412 | 1.00 | 38.07 |
| ATOM | 17876 | CD | GLU | 2659 | 44.583 | 13.585 | 72.476 | 1.00 | 40.89 |
| ATOM | 17877 | OE1 | GLU | 2659 | 44.763 | 12.360 | 72.642 | 1.00 | 41.36 |
| ATOM | 17878 | OE2 | GLU | 2659 | 45.156 | 14.245 | 71.582 | 1.00 | 41.02 |
| ATOM | 17879 | C | GLU | 2659 | 41.043 | 14.746 | 71.874 | 1.00 | 33.45 |
| ATOM | 17880 | O | GLU | 2659 | 41.012 | 13.514 | 71.823 | 1.00 | 33.66 |
| ATOM | 17881 | N | GLU | 2660 | 39.981 | 15.484 | 72.193 | 1.00 | 34.34 |
| ATOM | 17882 | CA | GLU | 2660 | 38.692 | 14.869 | 72.507 | 1.00 | 34.69 |
| ATOM | 17883 | CB | GLU | 2660 | 37.738 | 15.890 | 73.136 | 1.00 | 36.58 |
| ATOM | 17884 | CG | GLU | 2660 | 38.416 | 17.063 | 73.818 | 1.00 | 41.62 |
| ATOM | 17885 | CD | GLU | 2660 | 38.836 | 18.147 | 72.839 | 1.00 | 43.50 |
| ATOM | 17886 | OE1 | GLU | 2660 | 37.945 | 18.737 | 72.186 | 1.00 | 43.66 |
| ATOM | 17887 | OE2 | GLU | 2660 | 40.055 | 18.409 | 72.727 | 1.00 | 44.50 |
| ATOM | 17888 | C | GLU | 2660 | 38.057 | 14.328 | 71.233 | 1.00 | 33.13 |
| ATOM | 17889 | O | GLU | 2660 | 37.165 | 13.482 | 71.287 | 1.00 | 33.50 |
| ATOM | 17890 | N | HIS | 2661 | 38.526 | 14.827 | 70.093 | 1.00 | 31.20 |
| ATOM | 17891 | CA | HIS | 2661 | 38.010 | 14.415 | 68.792 | 1.00 | 29.37 |
| ATOM | 17892 | CB | HIS | 2661 | 37.777 | 15.643 | 67.903 | 1.00 | 29.29 |
| ATOM | 17893 | CG | HIS | 2661 | 36.937 | 16.708 | 68.537 | 1.00 | 29.26 |
| ATOM | 17894 | CD2 | HIS | 2661 | 37.231 | 17.984 | 68.884 | 1.00 | 28.56 |
| ATOM | 17895 | ND1 | HIS | 2661 | 35.613 | 16.517 | 68.868 | 1.00 | 29.92 |
| ATOM | 17896 | CE1 | HIS | 2661 | 35.126 | 17.630 | 69.391 | 1.00 | 29.59 |
| ATOM | 17897 | NE2 | HIS | 2661 | 36.088 | 18.535 | 69.411 | 1.00 | 28.86 |
| ATOM | 17898 | C | HIS | 2661 | 38.983 | 13.480 | 68.076 | 1.00 | 28.74 |
| ATOM | 17899 | O | HIS | 2661 | 38.755 | 13.119 | 66.924 | 1.00 | 28.41 |
| ATOM | 17900 | N | SER | 2662 | 40.055 | 13.088 | 68.761 | 1.00 | 27.65 |
| ATOM | 17901 | CA | SER | 2662 | 41.077 | 12.226 | 68.171 | 1.00 | 27.75 |
| ATOM | 17902 | CB | SER | 2662 | 42.453 | 12.873 | 68.346 | 1.00 | 24.62 |
| ATOM | 17903 | OG | SER | 2662 | 42.507 | 14.139 | 67.714 | 1.00 | 24.83 |
| ATOM | 17904 | C | SER | 2662 | 41.128 | 10.800 | 68.711 | 1.00 | 27.87 |
| ATOM | 17905 | O | SER | 2662 | 40.798 | 10.541 | 69.872 | 1.00 | 28.57 |
| ATOM | 17906 | N | PHE | 2663 | 41.562 | 9.876 | 67.857 | 1.00 | 27.35 |
| ATOM | 17907 | CA | PHE | 2663 | 41.680 | 8.472 | 68.231 | 1.00 | 27.58 |
| ATOM | 17908 | CB | PHE | 2663 | 40.990 | 7.581 | 67.195 | 1.00 | 28.72 |
| ATOM | 17909 | CG | PHE | 2663 | 39.507 | 7.795 | 67.099 | 1.00 | 28.64 |
| ATOM | 17910 | CD1 | PHE | 2663 | 38.948 | 8.405 | 65.980 | 1.00 | 28.22 |
| ATOM | 17911 | CD2 | PHE | 2663 | 38.664 | 7.369 | 68.123 | 1.00 | 28.60 |
| ATOM | 17912 | CE1 | PHE | 2663 | 37.568 | 8.585 | 65.879 | 1.00 | 28.53 |
| ATOM | 17913 | CE2 | PHE | 2663 | 37.284 | 7.545 | 68.033 | 1.00 | 28.38 |
| ATOM | 17914 | CZ | PHE | 2663 | 36.736 | 8.152 | 66.909 | 1.00 | 27.60 |
| ATOM | 17915 | C | PHE | 2663 | 43.147 | 8.070 | 68.337 | 1.00 | 29.19 |
| ATOM | 17916 | O | PHE | 2663 | 44.009 | 8.662 | 67.693 | 1.00 | 28.67 |
| ATOM | 17917 | N | HIS | 2664 | 43.425 | 7.061 | 69.154 | 1.00 | 30.62 |
| ATOM | 17918 | CA | HIS | 2664 | 44.793 | 6.580 | 69.335 | 1.00 | 33.11 |
| ATOM | 17919 | CB | HIS | 2664 | 45.390 | 7.136 | 70.631 | 1.00 | 32.47 |
| ATOM | 17920 | CG | HIS | 2664 | 45.696 | 8.600 | 70.569 | 1.00 | 31.82 |
| ATOM | 17921 | CD2 | HIS | 2664 | 45.143 | 9.657 | 71.209 | 1.00 | 31.31 |
| ATOM | 17922 | ND1 | HIS | 2664 | 46.670 | 9.119 | 69.743 | 1.00 | 31.66 |
| ATOM | 17923 | CE1 | HIS | 2664 | 46.704 | 10.433 | 69.877 | 1.00 | 32.26 |
| ATOM | 17924 | NE2 | HIS | 2664 | 45.787 | 10.785 | 70.761 | 1.00 | 31.44 |
| ATOM | 17925 | C | HIS | 2664 | 44.850 | 5.060 | 69.347 | 1.00 | 34.16 |
| ATOM | 17926 | O | HIS | 2664 | 45.924 | 4.517 | 69.013 | 1.00 | 34.77 |
| ATOM | 17927 | OXT | HIS | 2664 | 43.825 | 4.436 | 69.698 | 1.00 | 34.27 |
| ATOM | 17928 | C1 | KPL | 2665 | 32.243 | 11.877 | 55.862 | 1.00 | 40.79 |
| ATOM | 17929 | C2 | KPL | 2665 | 32.961 | 11.755 | 54.501 | 1.00 | 41.02 |
| ATOM | 17930 | C3 | KPL | 2665 | 33.077 | 13.146 | 53.870 | 1.00 | 41.20 |
| ATOM | 17931 | C4 | KPL | 2665 | 34.387 | 11.202 | 54.712 | 1.00 | 42.66 |
| ATOM | 17932 | O1 | KPL | 2665 | 34.336 | 9.899 | 55.310 | 1.00 | 45.13 |
| ATOM | 17933 | C5 | KPL | 2665 | 32.150 | 10.836 | 53.550 | 1.00 | 39.59 |
| ATOM | 17934 | O2 | KPL | 2665 | 32.663 | 9.838 | 53.081 | 1.00 | 38.96 |
| ATOM | 17935 | C6 | KPL | 2665 | 30.715 | 11.146 | 53.180 | 1.00 | 37.68 |
| ATOM | 17936 | O3 | KPL | 2665 | 30.159 | 12.135 | 53.620 | 1.00 | 35.87 |
| ATOM | 17937 | O4 | KPL | 2665 | 30.039 | 10.319 | 52.357 | 1.00 | 34.01 |
| ATOM | 17938 | CB | MET | 2701 | 34.899 | 19.058 | −4.231 | 1.00 | 73.64 |
| ATOM | 17939 | CG | MET | 2701 | 35.731 | 17.961 | −3.594 | 1.00 | 74.75 |
| ATOM | 17940 | SD | MET | 2701 | 37.148 | 17.478 | −4.583 | 1.00 | 76.63 |
| ATOM | 17941 | CE | MET | 2701 | 36.494 | 16.017 | −5.398 | 1.00 | 76.51 |
| ATOM | 17942 | C | MET | 2701 | 32.655 | 18.015 | −4.580 | 1.00 | 71.20 |
| ATOM | 17943 | O | MET | 2701 | 31.627 | 18.329 | −5.182 | 1.00 | 71.65 |
| ATOM | 17944 | N | MET | 2701 | 33.341 | 18.824 | −2.319 | 1.00 | 71.84 |
| ATOM | 17945 | CA | MET | 2701 | 33.434 | 19.060 | −3.787 | 1.00 | 72.13 |
| ATOM | 17946 | N | LYS | 2702 | 33.136 | 16.777 | −4.578 | 1.00 | 69.46 |
| ATOM | 17947 | CA | LYS | 2702 | 32.480 | 15.710 | −5.314 | 1.00 | 67.58 |
| ATOM | 17948 | CB | LYS | 2702 | 33.180 | 15.500 | −6.657 | 1.00 | 68.24 |
| ATOM | 17949 | CG | LYS | 2702 | 33.257 | 16.745 | −7.532 | 1.00 | 69.09 |
| ATOM | 17950 | CD | LYS | 2702 | 31.884 | 17.196 | −8.031 | 1.00 | 69.83 |
| ATOM | 17951 | CE | LYS | 2702 | 31.288 | 16.208 | −9.025 | 1.00 | 70.29 |
| ATOM | 17952 | NZ | LYS | 2702 | 29.978 | 16.690 | −9.549 | 1.00 | 70.64 |
| ATOM | 17953 | C | LYS | 2702 | 32.359 | 14.356 | −4.586 | 1.00 | 65.56 |
| ATOM | 17954 | O | LYS | 2702 | 32.309 | 13.312 | −5.230 | 1.00 | 66.40 |
| ATOM | 17955 | N | PRO | 2703 | 32.359 | 14.356 | −3.236 | 1.00 | 62.79 |
| ATOM | 17956 | CD | PRO | 2703 | 31.899 | 13.174 | −2.477 | 1.00 | 62.15 |
| ATOM | 17957 | CA | PRO | 2703 | 32.399 | 15.527 | −2.353 | 1.00 | 60.20 |
| ATOM | 17958 | CB | PRO | 2703 | 31.472 | 15.124 | −1.221 | 1.00 | 60.98 |
| ATOM | 17959 | CG | PRO | 2703 | 31.824 | 13.700 | −1.045 | 1.00 | 61.24 |
| ATOM | 17960 | C | PRO | 2703 | 33.838 | 15.784 | −1.867 | 1.00 | 57.10 |
| ATOM | 17961 | O | PRO | 2703 | 34.808 | 15.402 | −2.523 | 1.00 | 56.61 |
| ATOM | 17962 | N | THR | 2704 | 33.954 | 16.429 | −0.711 | 1.00 | 54.53 |
| ATOM | 17963 | CA | THR | 2704 | 35.261 | 16.729 | −0.126 | 1.00 | 51.38 |
| ATOM | 17964 | CB | THR | 2704 | 35.267 | 18.095 | 0.603 | 1.00 | 51.73 |
| ATOM | 17965 | OG1 | THR | 2704 | 34.978 | 19.147 | −0.328 | 1.00 | 52.06 |
| ATOM | 17966 | CG2 | THR | 2704 | 36.630 | 18.346 | 1.240 | 1.00 | 50.43 |
| ATOM | 17967 | C | THR | 2704 | 35.628 | 15.647 | 0.884 | 1.00 | 49.86 |
| ATOM | 17968 | O | THR | 2704 | 35.041 | 15.571 | 1.963 | 1.00 | 48.30 |
| ATOM | 17969 | N | THR | 2705 | 36.605 | 14.819 | 0.532 | 1.00 | 48.07 |
| ATOM | 17970 | CA | THR | 2705 | 37.040 | 13.743 | 1.410 | 1.00 | 47.18 |
| ATOM | 17971 | CB | THR | 2705 | 37.087 | 12.397 | 0.664 | 1.00 | 46.54 |
| ATOM | 17972 | OG1 | THR | 2705 | 38.071 | 12.459 | −0.375 | 1.00 | 45.99 |
| ATOM | 17973 | CG2 | THR | 2705 | 35.728 | 12.081 | 0.051 | 1.00 | 46.51 |
| ATOM | 17974 | C | THR | 2705 | 38.422 | 14.017 | 1.995 | 1.00 | 46.48 |
| ATOM | 17975 | O | THR | 2705 | 39.061 | 15.016 | 1.664 | 1.00 | 45.98 |
| ATOM | 17976 | N | ILE | 2706 | 38.875 | 13.119 | 2.864 | 1.00 | 45.87 |
| ATOM | 17977 | CA | ILE | 2706 | 40.180 | 13.245 | 3.502 | 1.00 | 45.37 |
| ATOM | 17978 | CB | ILE | 2706 | 40.461 | 12.049 | 4.438 | 1.00 | 45.76 |
| ATOM | 17979 | CG2 | ILE | 2706 | 41.744 | 12.292 | 5.223 | 1.00 | 45.05 |
| ATOM | 17980 | CG1 | ILE | 2706 | 39.287 | 11.852 | 5.400 | 1.00 | 47.29 |
| ATOM | 17981 | CD1 | ILE | 2706 | 39.380 | 10.579 | 6.232 | 1.00 | 47.98 |
| ATOM | 17982 | C | ILE | 2706 | 41.278 | 13.293 | 2.443 | 1.00 | 45.82 |
| ATOM | 17983 | O | ILE | 2706 | 42.361 | 13.834 | 2.676 | 1.00 | 44.79 |
| ATOM | 17984 | N | SER | 2707 | 40.992 | 12.719 | 1.278 | 1.00 | 45.94 |
| ATOM | 17985 | CA | SER | 2707 | 41.949 | 12.697 | 0.174 | 1.00 | 46.20 |
| ATOM | 17986 | CB | SER | 2707 | 41.338 | 11.997 | −1.045 | 1.00 | 46.03 |
| ATOM | 17987 | OG | SER | 2707 | 41.014 | 10.650 | −0.758 | 1.00 | 47.96 |
| ATOM | 17988 | C | SER | 2707 | 42.380 | 14.110 | −0.214 | 1.00 | 45.55 |
| ATOM | 17989 | O | SER | 2707 | 43.555 | 14.357 | −0.489 | 1.00 | 44.61 |
| ATOM | 17990 | N | LEU | 2708 | 41.423 | 15.032 | −0.236 | 1.00 | 45.78 |
| ATOM | 17991 | CA | LEU | 2708 | 41.699 | 16.422 | −0.590 | 1.00 | 46.61 |
| ATOM | 17992 | CB | LEU | 2708 | 40.410 | 17.250 | −0.538 | 1.00 | 47.45 |
| ATOM | 17993 | CG | LEU | 2708 | 39.904 | 17.852 | −1.853 | 1.00 | 47.62 |
| ATOM | 17994 | CD1 | LEU | 2708 | 38.612 | 18.619 | −1.609 | 1.00 | 48.54 |
| ATOM | 17995 | CD2 | LEU | 2708 | 40.960 | 18.772 | −2.436 | 1.00 | 48.50 |
| ATOM | 17996 | C | LEU | 2708 | 42.741 | 17.051 | 0.333 | 1.00 | 46.79 |
| ATOM | 17997 | O | LEU | 2708 | 43.699 | 17.671 | −0.133 | 1.00 | 46.13 |
| ATOM | 17998 | N | LEU | 2709 | 42.549 | 16.890 | 1.640 | 1.00 | 47.03 |
| ATOM | 17999 | CA | LEU | 2709 | 43.469 | 17.452 | 2.626 | 1.00 | 48.38 |
| ATOM | 18000 | CB | LEU | 2709 | 42.935 | 17.231 | 4.044 | 1.00 | 47.81 |
| ATOM | 18001 | CG | LEU | 2709 | 41.545 | 17.782 | 4.367 | 1.00 | 48.04 |
| ATOM | 18002 | CD1 | LEU | 2709 | 41.209 | 17.497 | 5.827 | 1.00 | 46.71 |
| ATOM | 18003 | CD2 | LEU | 2709 | 41.510 | 19.276 | 4.098 | 1.00 | 48.90 |
| ATOM | 18004 | C | LEU | 2709 | 44.854 | 16.829 | 2.505 | 1.00 | 49.28 |
| ATOM | 18005 | O | LEU | 2709 | 45.861 | 17.455 | 2.835 | 1.00 | 48.60 |
| ATOM | 18006 | N | GLN | 2710 | 44.895 | 15.589 | 2.032 | 1.00 | 50.99 |
| ATOM | 18007 | CA | GLN | 2710 | 46.154 | 14.880 | 1.867 | 1.00 | 52.96 |
| ATOM | 18008 | CB | GLN | 2710 | 45.893 | 13.391 | 1.632 | 1.00 | 54.28 |
| ATOM | 18009 | CG | GLN | 2710 | 47.137 | 12.520 | 1.688 | 1.00 | 57.12 |
| ATOM | 18010 | CD | GLN | 2710 | 47.721 | 12.419 | 3.087 | 1.00 | 58.97 |
| ATOM | 18011 | OE1 | GLN | 2710 | 48.102 | 13.423 | 3.692 | 1.00 | 60.04 |
| ATOM | 18012 | NE2 | GLN | 2710 | 47.794 | 11.198 | 3.607 | 1.00 | 59.77 |
| ATOM | 18013 | C | GLN | 2710 | 46.909 | 15.476 | 0.683 | 1.00 | 53.46 |
| ATOM | 18014 | O | GLN | 2710 | 48.096 | 15.790 | 0.787 | 1.00 | 53.06 |
| ATOM | 18015 | N | LYS | 2711 | 46.214 | 15.635 | −0.441 | 1.00 | 53.98 |
| ATOM | 18016 | CA | LYS | 2711 | 46.824 | 16.205 | −1.636 | 1.00 | 54.78 |
| ATOM | 18017 | CB | LYS | 2711 | 45.817 | 16.278 | −2.789 | 1.00 | 55.24 |
| ATOM | 18018 | CG | LYS | 2711 | 46.421 | 16.833 | −4.076 | 1.00 | 55.62 |
| ATOM | 18019 | CD | LYS | 2711 | 45.396 | 17.536 | −4.955 | 1.00 | 57.13 |
| ATOM | 18020 | CE | LYS | 2711 | 44.346 | 16.586 | −5.502 | 1.00 | 57.37 |
| ATOM | 18021 | NZ | LYS | 2711 | 43.404 | 17.307 | −6.407 | 1.00 | 58.73 |
| ATOM | 18022 | C | LYS | 2711 | 47.318 | 17.614 | −1.332 | 1.00 | 54.66 |
| ATOM | 18023 | O | LYS | 2711 | 48.382 | 18.025 | −1.792 | 1.00 | 55.31 |
| ATOM | 18024 | N | TYR | 2712 | 46.531 | 18.354 | −0.558 | 1.00 | 54.70 |
| ATOM | 18025 | CA | TYR | 2712 | 46.881 | 19.721 | −0.193 | 1.00 | 54.60 |
| ATOM | 18026 | CB | TYR | 2712 | 45.796 | 20.328 | 0.698 | 1.00 | 54.84 |
| ATOM | 18027 | CG | TYR | 2712 | 44.590 | 20.842 | −0.059 | 1.00 | 55.24 |
| ATOM | 18028 | CD1 | TYR | 2712 | 43.480 | 21.344 | 0.620 | 1.00 | 55.21 |
| ATOM | 18029 | CE1 | TYR | 2712 | 42.375 | 21.837 | −0.071 | 1.00 | 55.78 |
| ATOM | 18030 | CD2 | TYR | 2712 | 44.564 | 20.847 | −1.456 | 1.00 | 55.42 |
| ATOM | 18031 | CE2 | TYR | 2712 | 43.465 | 21.338 | −2.155 | 1.00 | 55.89 |
| ATOM | 18032 | CZ | TYR | 2712 | 42.376 | 21.830 | −1.458 | 1.00 | 55.09 |
| ATOM | 18033 | OH | TYR | 2712 | 41.290 | 22.313 | −2.145 | 1.00 | 55.85 |
| ATOM | 18034 | C | TYR | 2712 | 48.227 | 19.825 | 0.505 | 1.00 | 54.49 |
| ATOM | 18035 | O | TYR | 2712 | 49.023 | 20.709 | 0.191 | 1.00 | 54.80 |
| ATOM | 18036 | N | LYS | 2713 | 48.485 | 18.928 | 1.450 | 1.00 | 54.67 |
| ATOM | 18037 | CA | LYS | 2713 | 49.747 | 18.958 | 2.175 | 1.00 | 55.19 |
| ATOM | 18038 | CB | LYS | 2713 | 49.787 | 17.860 | 3.241 | 1.00 | 53.74 |
| ATOM | 18039 | CG | LYS | 2713 | 51.072 | 17.868 | 4.056 | 1.00 | 52.19 |
| ATOM | 18040 | CD | LYS | 2713 | 50.915 | 17.134 | 5.372 | 1.00 | 50.87 |
| ATOM | 18041 | CE | LYS | 2713 | 52.169 | 17.275 | 6.218 | 1.00 | 50.54 |
| ATOM | 18042 | NZ | LYS | 2713 | 51.974 | 16.777 | 7.605 | 1.00 | 49.42 |
| ATOM | 18043 | C | LYS | 2713 | 50.935 | 18.809 | 1.232 | 1.00 | 55.89 |
| ATOM | 18044 | O | LYS | 2713 | 51.998 | 19.380 | 1.471 | 1.00 | 56.25 |
| ATOM | 18045 | N | GLN | 2714 | 50.753 | 18.042 | 0.161 | 1.00 | 57.18 |
| ATOM | 18046 | CA | GLN | 2714 | 51.820 | 17.844 | −0.812 | 1.00 | 58.36 |
| ATOM | 18047 | CB | GLN | 2714 | 51.438 | 16.763 | −1.819 | 1.00 | 59.42 |
| ATOM | 18048 | CG | GLN | 2714 | 51.254 | 15.390 | −1.216 | 1.00 | 61.57 |
| ATOM | 18049 | CD | GLN | 2714 | 50.997 | 14.336 | −2.270 | 1.00 | 62.96 |
| ATOM | 18050 | OE1 | GLN | 2714 | 51.813 | 14.134 | −3.171 | 1.00 | 64.00 |
| ATOM | 18051 | NE2 | GLN | 2714 | 49.859 | 13.656 | −2.166 | 1.00 | 63.63 |
| ATOM | 18052 | C | GLN | 2714 | 52.073 | 19.146 | −1.551 | 1.00 | 58.50 |
| ATOM | 18053 | O | GLN | 2714 | 53.217 | 19.563 | −1.722 | 1.00 | 59.24 |
| ATOM | 18054 | N | GLU | 2715 | 50.992 | 19.785 | −1.986 | 1.00 | 58.54 |
| ATOM | 18055 | CA | GLU | 2715 | 51.082 | 21.042 | −2.714 | 1.00 | 58.89 |
| ATOM | 18056 | CB | GLU | 2715 | 49.783 | 21.294 | −3.477 | 1.00 | 59.02 |
| ATOM | 18057 | CG | GLU | 2715 | 49.372 | 20.144 | −4.373 | 1.00 | 59.98 |
| ATOM | 18058 | CD | GLU | 2715 | 48.032 | 20.381 | −5.034 | 1.00 | 60.87 |
| ATOM | 18059 | OE1 | GLU | 2715 | 47.061 | 20.683 | −4.309 | 1.00 | 61.95 |
| ATOM | 18060 | OE2 | GLU | 2715 | 47.946 | 20.260 | −6.274 | 1.00 | 61.16 |
| ATOM | 18061 | C | GLU | 2715 | 51.352 | 22.199 | −1.761 | 1.00 | 58.97 |
| ATOM | 18062 | O | GLU | 2715 | 51.366 | 23.360 | −2.169 | 1.00 | 59.41 |
| ATOM | 18063 | N | LYS | 2716 | 51.563 | 21.874 | −0.489 | 1.00 | 58.61 |
| ATOM | 18064 | CA | LYS | 2716 | 51.836 | 22.882 | 0.531 | 1.00 | 58.78 |
| ATOM | 18065 | CB | LYS | 2716 | 53.169 | 23.578 | 0.244 | 1.00 | 58.70 |
| ATOM | 18066 | CG | LYS | 2716 | 54.325 | 22.634 | −0.032 | 1.00 | 59.65 |
| ATOM | 18067 | CD | LYS | 2716 | 54.665 | 21.785 | 1.178 | 1.00 | 60.37 |
| ATOM | 18068 | CE | LYS | 2716 | 55.783 | 20.805 | 0.853 | 1.00 | 61.03 |
| ATOM | 18069 | NZ | LYS | 2716 | 57.003 | 21.501 | 0.359 | 1.00 | 60.83 |
| ATOM | 18070 | C | LYS | 2716 | 50.722 | 23.924 | 0.561 | 1.00 | 58.16 |
| ATOM | 18071 | O | LYS | 2716 | 50.926 | 25.047 | 1.020 | 1.00 | 58.70 |
| ATOM | 18072 | N | LYS | 2717 | 49.548 | 23.544 | 0.065 | 1.00 | 57.44 |
| ATOM | 18073 | CA | LYS | 2717 | 48.400 | 24.442 | 0.024 | 1.00 | 56.21 |
| ATOM | 18074 | CB | LYS | 2717 | 47.502 | 24.083 | −1.167 | 1.00 | 57.20 |
| ATOM | 18075 | CG | LYS | 2717 | 46.344 | 25.048 | −1.400 | 1.00 | 58.48 |
| ATOM | 18076 | CD | LYS | 2717 | 45.574 | 24.717 | −2.679 | 1.00 | 59.26 |
| ATOM | 18077 | CE | LYS | 2717 | 46.441 | 24.883 | −3.925 | 1.00 | 60.27 |
| ATOM | 18078 | NZ | LYS | 2717 | 45.681 | 24.594 | −5.174 | 1.00 | 59.88 |
| ATOM | 18079 | C | LYS | 2717 | 47.604 | 24.368 | 1.326 | 1.00 | 55.15 |
| ATOM | 18080 | O | LYS | 2717 | 46.819 | 23.443 | 1.536 | 1.00 | 55.14 |
| ATOM | 18081 | N | ARG | 2718 | 47.820 | 25.350 | 2.197 | 1.00 | 53.27 |
| ATOM | 18082 | CA | ARG | 2718 | 47.135 | 25.413 | 3.483 | 1.00 | 52.15 |
| ATOM | 18083 | CB | ARG | 2718 | 47.728 | 26.537 | 4.334 | 1.00 | 52.42 |
| ATOM | 18084 | CG | ARG | 2718 | 49.122 | 26.226 | 4.849 | 1.00 | 53.20 |
| ATOM | 18085 | CD | ARG | 2718 | 49.749 | 27.416 | 5.548 | 1.00 | 54.76 |
| ATOM | 18086 | NE | ARG | 2718 | 50.135 | 28.463 | 4.605 | 1.00 | 55.74 |
| ATOM | 18087 | CZ | ARG | 2718 | 50.810 | 29.557 | 4.944 | 1.00 | 56.58 |
| ATOM | 18088 | NH1 | ARG | 2718 | 51.173 | 29.750 | 6.204 | 1.00 | 56.22 |
| ATOM | 18089 | NH2 | ARG | 2718 | 51.131 | 30.454 | 4.021 | 1.00 | 57.45 |
| ATOM | 18090 | C | ARG | 2718 | 45.632 | 25.612 | 3.328 | 1.00 | 50.84 |
| ATOM | 18091 | O | ARG | 2718 | 45.182 | 26.468 | 2.565 | 1.00 | 50.29 |
| ATOM | 18092 | N | PHE | 2719 | 44.863 | 24.816 | 4.067 | 1.00 | 48.72 |
| ATOM | 18093 | CA | PHE | 2719 | 43.406 | 24.872 | 4.016 | 1.00 | 46.09 |
| ATOM | 18094 | CB | PHE | 2719 | 42.856 | 23.483 | 3.674 | 1.00 | 47.13 |
| ATOM | 18095 | CG | PHE | 2719 | 43.372 | 22.390 | 4.565 | 1.00 | 46.19 |
| ATOM | 18096 | CD1 | PHE | 2719 | 42.764 | 22.122 | 5.788 | 1.00 | 46.63 |
| ATOM | 18097 | CD2 | PHE | 2719 | 44.483 | 21.643 | 4.193 | 1.00 | 46.52 |
| ATOM | 18098 | CE1 | PHE | 2719 | 43.257 | 21.124 | 6.628 | 1.00 | 46.28 |
| ATOM | 18099 | CE2 | PHE | 2719 | 44.984 | 20.644 | 5.024 | 1.00 | 46.72 |
| ATOM | 18100 | CZ | PHE | 2719 | 44.369 | 20.383 | 6.245 | 1.00 | 46.49 |
| ATOM | 18101 | C | PHE | 2719 | 42.788 | 25.375 | 5.318 | 1.00 | 44.37 |
| ATOM | 18102 | O | PHE | 2719 | 43.406 | 25.307 | 6.381 | 1.00 | 43.11 |
| ATOM | 18103 | N | ALA | 2720 | 41.561 | 25.879 | 5.223 | 1.00 | 42.30 |
| ATOM | 18104 | CA | ALA | 2720 | 40.848 | 26.403 | 6.381 | 1.00 | 41.78 |
| ATOM | 18105 | CB | ALA | 2720 | 40.263 | 27.772 | 6.049 | 1.00 | 41.45 |
| ATOM | 18106 | C | ALA | 2720 | 39.738 | 25.467 | 6.860 | 1.00 | 40.79 |
| ATOM | 18107 | O | ALA | 2720 | 39.120 | 24.756 | 6.065 | 1.00 | 39.99 |
| ATOM | 18108 | N | THR | 2721 | 39.495 | 25.481 | 8.169 | 1.00 | 40.42 |
| ATOM | 18109 | CA | THR | 2721 | 38.459 | 24.659 | 8.797 | 1.00 | 38.92 |
| ATOM | 18110 | CB | THR | 2721 | 39.074 | 23.502 | 9.607 | 1.00 | 39.08 |
| ATOM | 18111 | OG1 | THR | 2721 | 40.006 | 22.786 | 8.787 | 1.00 | 41.05 |
| ATOM | 18112 | CG2 | THR | 2721 | 37.986 | 22.545 | 10.073 | 1.00 | 41.39 |
| ATOM | 18113 | C | THR | 2721 | 37.660 | 25.543 | 9.757 | 1.00 | 37.36 |
| ATOM | 18114 | O | THR | 2721 | 38.181 | 26.531 | 10.269 | 1.00 | 37.24 |
| ATOM | 18115 | N | ILE | 2722 | 36.404 | 25.188 | 10.010 | 1.00 | 34.57 |
| ATOM | 18116 | CA | ILE | 2722 | 35.575 | 25.983 | 10.907 | 1.00 | 33.33 |
| ATOM | 18117 | CB | ILE | 2722 | 34.856 | 27.114 | 10.121 | 1.00 | 33.46 |
| ATOM | 18118 | CG2 | ILE | 2722 | 33.767 | 26.519 | 9.232 | 1.00 | 32.91 |
| ATOM | 18119 | CG1 | ILE | 2722 | 34.256 | 28.136 | 11.091 | 1.00 | 33.34 |
| ATOM | 18120 | CD1 | ILE | 2722 | 33.727 | 29.396 | 10.411 | 1.00 | 33.25 |
| ATOM | 18121 | C | ILE | 2722 | 34.539 | 25.121 | 11.633 | 1.00 | 33.03 |
| ATOM | 18122 | O | ILE | 2722 | 34.165 | 24.048 | 11.160 | 1.00 | 32.21 |
| ATOM | 18123 | N | THR | 2723 | 34.089 | 25.591 | 12.791 | 1.00 | 31.25 |
| ATOM | 18124 | CA | THR | 2723 | 33.099 | 24.861 | 13.563 | 1.00 | 30.50 |
| ATOM | 18125 | CB | THR | 2723 | 33.120 | 25.275 | 15.044 | 1.00 | 31.78 |
| ATOM | 18126 | OG1 | THR | 2723 | 32.810 | 26.669 | 15.150 | 1.00 | 33.34 |
| ATOM | 18127 | CG2 | THR | 2723 | 34.489 | 25.014 | 15.648 | 1.00 | 31.14 |
| ATOM | 18128 | C | THR | 2723 | 31.716 | 25.143 | 12.996 | 1.00 | 29.87 |
| ATOM | 18129 | O | THR | 2723 | 31.491 | 26.174 | 12.360 | 1.00 | 29.14 |
| ATOM | 18130 | N | ALA | 2724 | 30.794 | 24.214 | 13.218 | 1.00 | 27.82 |
| ATOM | 18131 | CA | ALA | 2724 | 29.428 | 24.364 | 12.739 | 1.00 | 26.76 |
| ATOM | 18132 | CB | ALA | 2724 | 29.332 | 23.966 | 11.271 | 1.00 | 26.45 |
| ATOM | 18133 | C | ALA | 2724 | 28.556 | 23.462 | 13.594 | 1.00 | 26.25 |
| ATOM | 18134 | O | ALA | 2724 | 28.991 | 22.388 | 13.999 | 1.00 | 24.53 |
| ATOM | 18135 | N | TYR | 2725 | 27.336 | 23.905 | 13.877 | 1.00 | 25.34 |
| ATOM | 18136 | CA | TYR | 2725 | 26.422 | 23.126 | 14.702 | 1.00 | 25.41 |
| ATOM | 18137 | CB | TYR | 2725 | 26.441 | 23.634 | 16.145 | 1.00 | 25.02 |
| ATOM | 18138 | CG | TYR | 2725 | 27.818 | 23.940 | 16.681 | 1.00 | 25.72 |
| ATOM | 18139 | CD1 | TYR | 2725 | 28.264 | 25.257 | 16.791 | 1.00 | 27.23 |
| ATOM | 18140 | CE1 | TYR | 2725 | 29.534 | 25.548 | 17.278 | 1.00 | 27.51 |
| ATOM | 18141 | CD2 | TYR | 2725 | 28.678 | 22.916 | 17.071 | 1.00 | 24.40 |
| ATOM | 18142 | CE2 | TYR | 2725 | 29.951 | 23.195 | 17.557 | 1.00 | 27.21 |
| ATOM | 18143 | CZ | TYR | 2725 | 30.372 | 24.515 | 17.659 | 1.00 | 27.25 |
| ATOM | 18144 | OH | TYR | 2725 | 31.632 | 24.797 | 18.149 | 1.00 | 29.59 |
| ATOM | 18145 | C | TYR | 2725 | 24.996 | 23.188 | 14.181 | 1.00 | 25.48 |
| ATOM | 18146 | O | TYR | 2725 | 24.073 | 22.716 | 14.847 | 1.00 | 25.90 |
| ATOM | 18147 | N | ASP | 2726 | 24.812 | 23.773 | 12.999 | 1.00 | 24.99 |
| ATOM | 18148 | CA | ASP | 2726 | 23.475 | 23.895 | 12.424 | 1.00 | 25.25 |
| ATOM | 18149 | CB | ASP | 2726 | 22.736 | 25.085 | 13.049 | 1.00 | 24.48 |
| ATOM | 18150 | CG | ASP | 2726 | 23.368 | 26.424 | 12.701 | 1.00 | 27.30 |
| ATOM | 18151 | OD1 | ASP | 2726 | 23.278 | 26.844 | 11.529 | 1.00 | 27.92 |
| ATOM | 18152 | OD2 | ASP | 2726 | 23.954 | 27.055 | 13.608 | 1.00 | 26.94 |
| ATOM | 18153 | C | ASP | 2726 | 23.463 | 24.023 | 10.906 | 1.00 | 25.69 |
| ATOM | 18154 | O | ASP | 2726 | 24.482 | 24.317 | 10.282 | 1.00 | 25.88 |
| ATOM | 18155 | N | TYR | 2727 | 22.289 | 23.792 | 10.329 | 1.00 | 25.18 |
| ATOM | 18156 | CA | TYR | 2727 | 22.080 | 23.855 | 8.890 | 1.00 | 27.78 |
| ATOM | 18157 | CB | TYR | 2727 | 20.607 | 23.601 | 8.575 | 1.00 | 29.33 |
| ATOM | 18158 | CG | TYR | 2727 | 20.215 | 23.942 | 7.156 | 1.00 | 32.36 |
| ATOM | 18159 | CD1 | TYR | 2727 | 20.418 | 23.035 | 6.117 | 1.00 | 33.43 |
| ATOM | 18160 | CE1 | TYR | 2727 | 20.049 | 23.348 | 4.808 | 1.00 | 35.74 |
| ATOM | 18161 | CD2 | TYR | 2727 | 19.636 | 25.177 | 6.853 | 1.00 | 32.74 |
| ATOM | 18162 | CE2 | TYR | 2727 | 19.266 | 25.501 | 5.550 | 1.00 | 34.63 |
| ATOM | 18163 | CZ | TYR | 2727 | 19.473 | 24.581 | 4.535 | 1.00 | 34.95 |
| ATOM | 18164 | OH | TYR | 2727 | 19.090 | 24.890 | 3.250 | 1.00 | 36.38 |
| ATOM | 18165 | C | TYR | 2727 | 22.481 | 25.185 | 8.261 | 1.00 | 28.49 |
| ATOM | 18166 | O | TYR | 2727 | 23.244 | 25.219 | 7.296 | 1.00 | 26.62 |
| ATOM | 18167 | N | SER | 2728 | 21.940 | 26.273 | 8.798 | 1.00 | 29.50 |
| ATOM | 18168 | CA | SER | 2728 | 22.215 | 27.601 | 8.270 | 1.00 | 31.15 |
| ATOM | 18169 | CB | SER | 2728 | 21.606 | 28.670 | 9.174 | 1.00 | 30.32 |
| ATOM | 18170 | OG | SER | 2728 | 20.195 | 28.606 | 9.127 | 1.00 | 30.03 |
| ATOM | 18171 | C | SER | 2728 | 23.691 | 27.885 | 8.063 | 1.00 | 32.53 |
| ATOM | 18172 | O | SER | 2728 | 24.151 | 28.017 | 6.927 | 1.00 | 34.08 |
| ATOM | 18173 | N | PHE | 2729 | 24.441 | 27.979 | 9.152 | 1.00 | 33.01 |
| ATOM | 18174 | CA | PHE | 2729 | 25.860 | 28.263 | 9.026 | 1.00 | 34.03 |
| ATOM | 18175 | CB | PHE | 2729 | 26.514 | 28.379 | 10.402 | 1.00 | 32.92 |
| ATOM | 18176 | CG | PHE | 2729 | 26.244 | 29.689 | 11.084 | 1.00 | 33.06 |
| ATOM | 18177 | CD1 | PHE | 2729 | 25.190 | 29.824 | 11.979 | 1.00 | 32.75 |
| ATOM | 18178 | CD2 | PHE | 2729 | 27.041 | 30.800 | 10.816 | 1.00 | 33.37 |
| ATOM | 18179 | CE1 | PHE | 2729 | 24.934 | 31.046 | 12.601 | 1.00 | 33.58 |
| ATOM | 18180 | CE2 | PHE | 2729 | 26.793 | 32.024 | 11.430 | 1.00 | 33.46 |
| ATOM | 18181 | CZ | PHE | 2729 | 25.738 | 32.148 | 12.325 | 1.00 | 33.47 |
| ATOM | 18182 | C | PHE | 2729 | 26.598 | 27.241 | 8.171 | 1.00 | 34.90 |
| ATOM | 18183 | O | PHE | 2729 | 27.417 | 27.614 | 7.333 | 1.00 | 35.39 |
| ATOM | 18184 | N | ALA | 2730 | 26.310 | 25.958 | 8.367 | 1.00 | 34.77 |
| ATOM | 18185 | CA | ALA | 2730 | 26.968 | 24.919 | 7.582 | 1.00 | 34.81 |
| ATOM | 18186 | CB | ALA | 2730 | 26.409 | 23.544 | 7.949 | 1.00 | 34.81 |
| ATOM | 18187 | C | ALA | 2730 | 26.744 | 25.198 | 6.099 | 1.00 | 34.77 |
| ATOM | 18188 | O | ALA | 2730 | 27.638 | 25.002 | 5.275 | 1.00 | 34.75 |
| ATOM | 18189 | N | LYS | 2731 | 25.540 | 25.659 | 5.777 | 1.00 | 35.58 |
| ATOM | 18190 | CA | LYS | 2731 | 25.154 | 25.983 | 4.408 | 1.00 | 36.11 |
| ATOM | 18191 | CB | LYS | 2731 | 23.657 | 26.294 | 4.361 | 1.00 | 37.50 |
| ATOM | 18192 | CG | LYS | 2731 | 23.151 | 26.818 | 3.033 | 1.00 | 38.67 |
| ATOM | 18193 | CD | LYS | 2731 | 23.122 | 25.740 | 1.977 | 1.00 | 40.61 |
| ATOM | 18194 | CE | LYS | 2731 | 22.524 | 26.278 | 0.685 | 1.00 | 42.74 |
| ATOM | 18195 | NZ | LYS | 2731 | 22.418 | 25.227 | −0.361 | 1.00 | 43.93 |
| ATOM | 18196 | C | LYS | 2731 | 25.938 | 27.189 | 3.909 | 1.00 | 36.82 |
| ATOM | 18197 | O | LYS | 2731 | 26.426 | 27.204 | 2.777 | 1.00 | 35.95 |
| ATOM | 18198 | N | LEU | 2732 | 26.048 | 28.198 | 4.766 | 1.00 | 37.43 |
| ATOM | 18199 | CA | LEU | 2732 | 26.762 | 29.421 | 4.434 | 1.00 | 38.34 |
| ATOM | 18200 | CB | LEU | 2732 | 26.576 | 30.449 | 5.558 | 1.00 | 38.63 |
| ATOM | 18201 | CG | LEU | 2732 | 27.150 | 31.858 | 5.367 | 1.00 | 38.74 |
| ATOM | 18202 | CD1 | LEU | 2732 | 26.427 | 32.832 | 6.281 | 1.00 | 37.58 |
| ATOM | 18203 | CD2 | LEU | 2732 | 28.643 | 31.853 | 5.652 | 1.00 | 38.30 |
| ATOM | 18204 | C | LEU | 2732 | 28.247 | 29.163 | 4.179 | 1.00 | 38.90 |
| ATOM | 18205 | O | LEU | 2732 | 28.830 | 29.742 | 3.264 | 1.00 | 39.61 |
| ATOM | 18206 | N | PHE | 2733 | 28.861 | 28.292 | 4.976 | 1.00 | 38.38 |
| ATOM | 18207 | CA | PHE | 2733 | 30.278 | 27.992 | 4.793 | 1.00 | 39.32 |
| ATOM | 18208 | CB | PHE | 2733 | 30.828 | 27.199 | 5.984 | 1.00 | 38.73 |
| ATOM | 18209 | CG | PHE | 2733 | 30.623 | 27.870 | 7.314 | 1.00 | 38.08 |
| ATOM | 18210 | CD1 | PHE | 2733 | 30.813 | 29.241 | 7.459 | 1.00 | 37.35 |
| ATOM | 18211 | CD2 | PHE | 2733 | 30.267 | 27.123 | 8.430 | 1.00 | 36.25 |
| ATOM | 18212 | CE1 | PHE | 2733 | 30.652 | 29.856 | 8.698 | 1.00 | 36.41 |
| ATOM | 18213 | CE2 | PHE | 2733 | 30.104 | 27.729 | 9.671 | 1.00 | 36.51 |
| ATOM | 18214 | CZ | PHE | 2733 | 30.297 | 29.098 | 9.804 | 1.00 | 36.45 |
| ATOM | 18215 | C | PHE | 2733 | 30.509 | 27.192 | 3.514 | 1.00 | 39.89 |
| ATOM | 18216 | O | PHE | 2733 | 31.449 | 27.455 | 2.764 | 1.00 | 39.04 |
| ATOM | 18217 | N | ALA | 2734 | 29.642 | 26.213 | 3.274 | 1.00 | 40.69 |
| ATOM | 18218 | CA | ALA | 2734 | 29.746 | 25.366 | 2.094 | 1.00 | 42.63 |
| ATOM | 18219 | CB | ALA | 2734 | 28.712 | 24.246 | 2.166 | 1.00 | 41.54 |
| ATOM | 18220 | C | ALA | 2734 | 29.564 | 26.157 | 0.800 | 1.00 | 44.13 |
| ATOM | 18221 | O | ALA | 2734 | 30.273 | 25.926 | −0.177 | 1.00 | 44.26 |
| ATOM | 18222 | N | ASP | 2735 | 28.612 | 27.085 | 0.795 | 1.00 | 45.42 |
| ATOM | 18223 | CA | ASP | 2735 | 28.354 | 27.889 | −0.394 | 1.00 | 47.29 |
| ATOM | 18224 | CB | ASP | 2735 | 27.145 | 28.802 | −0.185 | 1.00 | 47.94 |
| ATOM | 18225 | CG | ASP | 2735 | 25.840 | 28.038 | −0.114 | 1.00 | 49.27 |
| ATOM | 18226 | OD1 | ASP | 2735 | 25.710 | 27.008 | −0.815 | 1.00 | 49.87 |
| ATOM | 18227 | OD2 | ASP | 2735 | 24.937 | 28.478 | 0.630 | 1.00 | 49.47 |
| ATOM | 18228 | C | ASP | 2735 | 29.555 | 28.738 | −0.794 | 1.00 | 47.96 |
| ATOM | 18229 | O | ASP | 2735 | 29.759 | 29.013 | −1.976 | 1.00 | 47.45 |
| ATOM | 18230 | N | GLU | 2736 | 30.343 | 29.154 | 0.193 | 1.00 | 48.95 |
| ATOM | 18231 | CA | GLU | 2736 | 31.520 | 29.976 | −0.062 | 1.00 | 50.11 |
| ATOM | 18232 | CB | GLU | 2736 | 31.884 | 30.780 | 1.187 | 1.00 | 50.79 |
| ATOM | 18233 | CG | GLU | 2736 | 30.787 | 31.710 | 1.663 | 1.00 | 51.69 |
| ATOM | 18234 | CD | GLU | 2736 | 30.351 | 32.690 | 0.594 | 1.00 | 52.87 |
| ATOM | 18235 | OE1 | GLU | 2736 | 31.200 | 33.477 | 0.129 | 1.00 | 54.23 |
| ATOM | 18236 | OE2 | GLU | 2736 | 29.160 | 32.673 | 0.219 | 1.00 | 52.97 |
| ATOM | 18237 | C | GLU | 2736 | 32.717 | 29.139 | −0.494 | 1.00 | 50.56 |
| ATOM | 18238 | O | GLU | 2736 | 33.330 | 29.405 | −1.528 | 1.00 | 51.77 |
| ATOM | 18239 | N | GLY | 2737 | 33.051 | 28.127 | 0.299 | 1.00 | 50.60 |
| ATOM | 18240 | CA | GLY | 2737 | 34.181 | 27.281 | −0.044 | 1.00 | 49.87 |
| ATOM | 18241 | C | GLY | 2737 | 34.630 | 26.374 | 1.082 | 1.00 | 49.32 |
| ATOM | 18242 | O | GLY | 2737 | 35.293 | 25.367 | 0.839 | 1.00 | 49.07 |
| ATOM | 18243 | N | LEU | 2738 | 34.281 | 26.735 | 2.313 | 1.00 | 49.01 |
| ATOM | 18244 | CA | LEU | 2738 | 34.646 | 25.936 | 3.475 | 1.00 | 48.38 |
| ATOM | 18245 | CB | LEU | 2738 | 34.252 | 26.658 | 4.765 | 1.00 | 48.66 |
| ATOM | 18246 | CG | LEU | 2738 | 35.241 | 27.691 | 5.301 | 1.00 | 47.93 |
| ATOM | 18247 | CD1 | LEU | 2738 | 34.628 | 28.435 | 6.473 | 1.00 | 47.98 |
| ATOM | 18248 | CD2 | LEU | 2738 | 36.523 | 26.992 | 5.726 | 1.00 | 49.25 |
| ATOM | 18249 | C | LEU | 2738 | 33.958 | 24.579 | 3.418 | 1.00 | 47.74 |
| ATOM | 18250 | O | LEU | 2738 | 32.781 | 24.457 | 3.757 | 1.00 | 48.29 |
| ATOM | 18251 | N | ASN | 2739 | 34.701 | 23.564 | 2.986 | 1.00 | 46.36 |
| ATOM | 18252 | CA | ASN | 2739 | 34.168 | 22.214 | 2.873 | 1.00 | 45.27 |
| ATOM | 18253 | CB | ASN | 2739 | 34.513 | 21.624 | 1.502 | 1.00 | 46.52 |
| ATOM | 18254 | CG | ASN | 2739 | 34.170 | 22.564 | 0.356 | 1.00 | 48.47 |
| ATOM | 18255 | OD1 | ASN | 2739 | 33.080 | 23.142 | 0.315 | 1.00 | 49.82 |
| ATOM | 18256 | ND2 | ASN | 2739 | 35.096 | 22.714 | −0.586 | 1.00 | 48.29 |
| ATOM | 18257 | C | ASN | 2739 | 34.733 | 21.319 | 3.975 | 1.00 | 43.36 |
| ATOM | 18258 | O | ASN | 2739 | 34.751 | 20.097 | 3.850 | 1.00 | 44.23 |
| ATOM | 18259 | N | VAL | 2740 | 35.196 | 21.938 | 5.054 | 1.00 | 40.65 |
| ATOM | 18260 | CA | VAL | 2740 | 35.753 | 21.200 | 6.179 | 1.00 | 38.58 |
| ATOM | 18261 | CB | VAL | 2740 | 37.294 | 21.276 | 6.180 | 1.00 | 39.42 |
| ATOM | 18262 | CG1 | VAL | 2740 | 37.863 | 20.397 | 7.282 | 1.00 | 38.41 |
| ATOM | 18263 | CG2 | VAL | 2740 | 37.836 | 20.840 | 4.826 | 1.00 | 39.41 |
| ATOM | 18264 | C | VAL | 2740 | 35.206 | 21.797 | 7.472 | 1.00 | 37.55 |
| ATOM | 18265 | O | VAL | 2740 | 35.670 | 22.843 | 7.932 | 1.00 | 36.45 |
| ATOM | 18266 | N | MET | 2741 | 34.213 | 21.128 | 8.052 | 1.00 | 35.79 |
| ATOM | 18267 | CA | MET | 2741 | 33.586 | 21.605 | 9.280 | 1.00 | 33.14 |
| ATOM | 18268 | CB | MET | 2741 | 32.097 | 21.858 | 9.019 | 1.00 | 33.81 |
| ATOM | 18269 | CG | MET | 2741 | 31.862 | 23.092 | 8.157 | 1.00 | 33.62 |
| ATOM | 18270 | SD | MET | 2741 | 30.216 | 23.250 | 7.476 | 1.00 | 35.47 |
| ATOM | 18271 | CE | MET | 2741 | 30.583 | 23.290 | 5.712 | 1.00 | 31.93 |
| ATOM | 18272 | C | MET | 2741 | 33.781 | 20.666 | 10.466 | 1.00 | 31.98 |
| ATOM | 18273 | O | MET | 2741 | 33.970 | 19.461 | 10.301 | 1.00 | 30.77 |
| ATOM | 18274 | N | LEU | 2742 | 33.737 | 21.228 | 11.666 | 1.00 | 30.44 |
| ATOM | 18275 | CA | LEU | 2742 | 33.927 | 20.442 | 12.870 | 1.00 | 29.16 |
| ATOM | 18276 | CB | LEU | 2742 | 35.268 | 20.818 | 13.517 | 1.00 | 31.59 |
| ATOM | 18277 | CG | LEU | 2742 | 35.690 | 20.194 | 14.857 | 1.00 | 34.91 |
| ATOM | 18278 | CD1 | LEU | 2742 | 34.985 | 20.907 | 15.998 | 1.00 | 36.28 |
| ATOM | 18279 | CD2 | LEU | 2742 | 35.382 | 18.698 | 14.872 | 1.00 | 35.47 |
| ATOM | 18280 | C | LEU | 2742 | 32.784 | 20.603 | 13.871 | 1.00 | 28.08 |
| ATOM | 18281 | O | LEU | 2742 | 32.471 | 21.710 | 14.315 | 1.00 | 26.39 |
| ATOM | 18282 | N | VAL | 2743 | 32.154 | 19.479 | 14.205 | 1.00 | 26.29 |
| ATOM | 18283 | CA | VAL | 2743 | 31.058 | 19.457 | 15.165 | 1.00 | 24.79 |
| ATOM | 18284 | CB | VAL | 2743 | 29.926 | 18.504 | 14.705 | 1.00 | 24.91 |
| ATOM | 18285 | CG1 | VAL | 2743 | 28.798 | 18.501 | 15.733 | 1.00 | 23.46 |
| ATOM | 18286 | CG2 | VAL | 2743 | 29.399 | 18.927 | 13.336 | 1.00 | 23.03 |
| ATOM | 18287 | C | VAL | 2743 | 31.660 | 18.942 | 16.468 | 1.00 | 25.50 |
| ATOM | 18288 | O | VAL | 2743 | 31.584 | 17.751 | 16.765 | 1.00 | 24.89 |
| ATOM | 18289 | N | GLY | 2744 | 32.271 | 19.846 | 17.234 | 1.00 | 25.53 |
| ATOM | 18290 | CA | GLY | 2744 | 32.909 | 19.460 | 18.482 | 1.00 | 24.69 |
| ATOM | 18291 | C | GLY | 2744 | 32.088 | 19.678 | 19.739 | 1.00 | 25.51 |
| ATOM | 18292 | O | GLY | 2744 | 31.083 | 20.389 | 19.727 | 1.00 | 23.34 |
| ATOM | 18293 | N | ASP | 2745 | 32.530 | 19.059 | 20.829 | 1.00 | 26.45 |
| ATOM | 18294 | CA | ASP | 2745 | 31.846 | 19.170 | 22.109 | 1.00 | 27.04 |
| ATOM | 18295 | CB | ASP | 2745 | 32.468 | 18.218 | 23.136 | 1.00 | 28.02 |
| ATOM | 18296 | CG | ASP | 2745 | 33.963 | 18.422 | 23.283 | 1.00 | 31.06 |
| ATOM | 18297 | OD1 | ASP | 2745 | 34.442 | 19.544 | 23.010 | 1.00 | 31.67 |
| ATOM | 18298 | OD2 | ASP | 2745 | 34.655 | 17.469 | 23.674 | 1.00 | 33.16 |
| ATOM | 18299 | C | ASP | 2745 | 31.888 | 20.599 | 22.646 | 1.00 | 27.84 |
| ATOM | 18300 | O | ASP | 2745 | 31.295 | 20.902 | 23.682 | 1.00 | 26.58 |
| ATOM | 18301 | N | SER | 2746 | 32.598 | 21.476 | 21.946 | 1.00 | 27.48 |
| ATOM | 18302 | CA | SER | 2746 | 32.675 | 22.864 | 22.368 | 1.00 | 26.36 |
| ATOM | 18303 | CB | SER | 2746 | 33.541 | 23.670 | 21.397 | 1.00 | 26.95 |
| ATOM | 18304 | OG | SER | 2746 | 33.076 | 23.552 | 20.064 | 1.00 | 29.19 |
| ATOM | 18305 | C | SER | 2746 | 31.254 | 23.417 | 22.398 | 1.00 | 27.45 |
| ATOM | 18306 | O | SER | 2746 | 30.946 | 24.342 | 23.154 | 1.00 | 26.43 |
| ATOM | 18307 | N | LEU | 2747 | 30.388 | 22.830 | 21.575 | 1.00 | 25.07 |
| ATOM | 18308 | CA | LEU | 2747 | 28.988 | 23.244 | 21.501 | 1.00 | 24.80 |
| ATOM | 18309 | CB | LEU | 2747 | 28.220 | 22.325 | 20.539 | 1.00 | 24.87 |
| ATOM | 18310 | CG | LEU | 2747 | 28.127 | 20.828 | 20.860 | 1.00 | 26.63 |
| ATOM | 18311 | CD1 | LEU | 2747 | 26.966 | 20.567 | 21.816 | 1.00 | 25.96 |
| ATOM | 18312 | CD2 | LEU | 2747 | 27.912 | 20.050 | 19.564 | 1.00 | 25.74 |
| ATOM | 18313 | C | LEU | 2747 | 28.343 | 23.215 | 22.887 | 1.00 | 22.90 |
| ATOM | 18314 | O | LEU | 2747 | 27.315 | 23.851 | 23.122 | 1.00 | 22.29 |
| ATOM | 18315 | N | GLY | 2748 | 28.952 | 22.473 | 23.805 | 1.00 | 22.88 |
| ATOM | 18316 | CA | GLY | 2748 | 28.414 | 22.396 | 25.151 | 1.00 | 25.99 |
| ATOM | 18317 | C | GLY | 2748 | 28.449 | 23.744 | 25.842 | 1.00 | 27.51 |
| ATOM | 18318 | O | GLY | 2748 | 27.725 | 23.975 | 26.810 | 1.00 | 27.90 |
| ATOM | 18319 | N | MET | 2749 | 29.291 | 24.641 | 25.342 | 1.00 | 29.54 |
| ATOM | 18320 | CA | MET | 2749 | 29.411 | 25.970 | 25.930 | 1.00 | 30.31 |
| ATOM | 18321 | CB | MET | 2749 | 30.884 | 26.294 | 26.194 | 1.00 | 32.91 |
| ATOM | 18322 | CG | MET | 2749 | 31.546 | 25.370 | 27.208 | 1.00 | 35.18 |
| ATOM | 18323 | SD | MET | 2749 | 33.294 | 25.754 | 27.464 | 1.00 | 42.41 |
| ATOM | 18324 | CE | MET | 2749 | 33.162 | 27.152 | 28.569 | 1.00 | 40.70 |
| ATOM | 18325 | C | MET | 2749 | 28.798 | 27.044 | 25.042 | 1.00 | 29.17 |
| ATOM | 18326 | O | MET | 2749 | 28.003 | 27.863 | 25.500 | 1.00 | 30.78 |
| ATOM | 18327 | N | THR | 2750 | 29.162 | 27.029 | 23.767 | 1.00 | 28.64 |
| ATOM | 18328 | CA | THR | 2750 | 28.662 | 28.016 | 22.827 | 1.00 | 28.34 |
| ATOM | 18329 | CB | THR | 2750 | 29.546 | 28.057 | 21.570 | 1.00 | 29.53 |
| ATOM | 18330 | OG1 | THR | 2750 | 29.124 | 29.130 | 20.718 | 1.00 | 33.35 |
| ATOM | 18331 | CG2 | THR | 2750 | 29.450 | 26.744 | 20.816 | 1.00 | 29.90 |
| ATOM | 18332 | C | THR | 2750 | 27.213 | 27.787 | 22.409 | 1.00 | 27.81 |
| ATOM | 18333 | O | THR | 2750 | 26.495 | 28.731 | 22.073 | 1.00 | 27.33 |
| ATOM | 18334 | N | VAL | 2751 | 26.779 | 26.533 | 22.425 | 1.00 | 27.07 |
| ATOM | 18335 | CA | VAL | 2751 | 25.411 | 26.220 | 22.033 | 1.00 | 26.65 |
| ATOM | 18336 | CB | VAL | 2751 | 25.380 | 25.004 | 21.076 | 1.00 | 27.34 |
| ATOM | 18337 | CG1 | VAL | 2751 | 23.945 | 24.661 | 20.717 | 1.00 | 25.72 |
| ATOM | 18338 | CG2 | VAL | 2751 | 26.182 | 25.316 | 19.817 | 1.00 | 27.61 |
| ATOM | 18339 | C | VAL | 2751 | 24.508 | 25.940 | 23.231 | 1.00 | 25.59 |
| ATOM | 18340 | O | VAL | 2751 | 23.459 | 26.565 | 23.380 | 1.00 | 25.62 |
| ATOM | 18341 | N | GLN | 2752 | 24.924 | 25.008 | 24.085 | 1.00 | 25.08 |
| ATOM | 18342 | CA | GLN | 2752 | 24.140 | 24.633 | 25.261 | 1.00 | 25.15 |
| ATOM | 18343 | CB | GLN | 2752 | 24.556 | 23.237 | 25.735 | 1.00 | 25.01 |
| ATOM | 18344 | CG | GLN | 2752 | 24.136 | 22.122 | 24.776 | 1.00 | 21.90 |
| ATOM | 18345 | CD | GLN | 2752 | 24.763 | 20.776 | 25.103 | 1.00 | 19.31 |
| ATOM | 18346 | OE1 | GLN | 2752 | 25.425 | 20.616 | 26.126 | 1.00 | 20.25 |
| ATOM | 18347 | NE2 | GLN | 2752 | 24.563 | 19.802 | 24.223 | 1.00 | 16.51 |
| ATOM | 18348 | C | GLN | 2752 | 24.248 | 25.637 | 26.406 | 1.00 | 27.38 |
| ATOM | 18349 | O | GLN | 2752 | 23.303 | 25.818 | 27.179 | 1.00 | 26.43 |
| ATOM | 18350 | N | GLY | 2753 | 25.401 | 26.286 | 26.522 | 1.00 | 27.57 |
| ATOM | 18351 | CA | GLY | 2753 | 25.577 | 27.273 | 27.574 | 1.00 | 29.69 |
| ATOM | 18352 | C | GLY | 2753 | 26.144 | 26.765 | 28.886 | 1.00 | 30.82 |
| ATOM | 18353 | O | GLY | 2753 | 25.954 | 27.394 | 29.928 | 1.00 | 30.56 |
| ATOM | 18354 | N | HIS | 2754 | 26.836 | 25.631 | 28.842 | 1.00 | 30.11 |
| ATOM | 18355 | CA | HIS | 2754 | 27.448 | 25.063 | 30.036 | 1.00 | 32.21 |
| ATOM | 18356 | CB | HIS | 2754 | 27.639 | 23.557 | 29.877 | 1.00 | 33.27 |
| ATOM | 18357 | CG | HIS | 2754 | 26.359 | 22.794 | 29.763 | 1.00 | 34.86 |
| ATOM | 18358 | CD2 | HIS | 2754 | 25.852 | 22.052 | 28.751 | 1.00 | 34.88 |
| ATOM | 18359 | ND1 | HIS | 2754 | 25.433 | 22.735 | 30.783 | 1.00 | 35.48 |
| ATOM | 18360 | CE1 | HIS | 2754 | 24.411 | 21.988 | 30.404 | 1.00 | 35.76 |
| ATOM | 18361 | NE2 | HIS | 2754 | 24.641 | 21.562 | 29.175 | 1.00 | 35.21 |
| ATOM | 18362 | C | HIS | 2754 | 28.808 | 25.721 | 30.258 | 1.00 | 33.24 |
| ATOM | 18363 | O | HIS | 2754 | 29.329 | 26.392 | 29.365 | 1.00 | 31.50 |
| ATOM | 18364 | N | ASP | 2755 | 29.381 | 25.511 | 31.442 | 1.00 | 34.08 |
| ATOM | 18365 | CA | ASP | 2755 | 30.678 | 26.084 | 31.795 | 1.00 | 35.48 |
| ATOM | 18366 | CB | ASP | 2755 | 30.789 | 26.256 | 33.319 | 1.00 | 38.54 |
| ATOM | 18367 | CG | ASP | 2755 | 30.687 | 24.941 | 34.070 | 1.00 | 40.66 |
| ATOM | 18368 | OD1 | ASP | 2755 | 31.598 | 24.093 | 33.934 | 1.00 | 44.22 |
| ATOM | 18369 | OD2 | ASP | 2755 | 29.694 | 24.749 | 34.805 | 1.00 | 43.84 |
| ATOM | 18370 | C | ASP | 2755 | 31.841 | 25.240 | 31.285 | 1.00 | 35.23 |
| ATOM | 18371 | O | ASP | 2755 | 32.999 | 25.650 | 31.363 | 1.00 | 36.04 |
| ATOM | 18372 | N | SER | 2756 | 31.531 | 24.055 | 30.770 | 1.00 | 33.43 |
| ATOM | 18373 | CA | SER | 2756 | 32.550 | 23.161 | 30.239 | 1.00 | 31.37 |
| ATOM | 18374 | CB | SER | 2756 | 33.111 | 22.252 | 31.339 | 1.00 | 29.97 |
| ATOM | 18375 | OG | SER | 2756 | 32.155 | 21.301 | 31.769 | 1.00 | 28.60 |
| ATOM | 18376 | C | SER | 2756 | 31.929 | 22.316 | 29.134 | 1.00 | 30.66 |
| ATOM | 18377 | O | SER | 2756 | 30.767 | 22.506 | 28.776 | 1.00 | 29.90 |
| ATOM | 18378 | N | THR | 2757 | 32.708 | 21.382 | 28.601 | 1.00 | 30.30 |
| ATOM | 18379 | CA | THR | 2757 | 32.236 | 20.520 | 27.526 | 1.00 | 29.72 |
| ATOM | 18380 | CB | THR | 2757 | 33.296 | 20.404 | 26.415 | 1.00 | 29.31 |
| ATOM | 18381 | OG1 | THR | 2757 | 34.491 | 19.832 | 26.957 | 1.00 | 27.82 |
| ATOM | 18382 | CG2 | THR | 2757 | 33.615 | 21.773 | 25.831 | 1.00 | 29.02 |
| ATOM | 18383 | C | THR | 2757 | 31.889 | 19.109 | 28.009 | 1.00 | 29.19 |
| ATOM | 18384 | O | THR | 2757 | 31.433 | 18.275 | 27.227 | 1.00 | 28.12 |
| ATOM | 18385 | N | LEU | 2758 | 32.107 | 18.842 | 29.293 | 1.00 | 28.20 |
| ATOM | 18386 | CA | LEU | 2758 | 31.824 | 17.519 | 29.849 | 1.00 | 27.55 |
| ATOM | 18387 | CB | LEU | 2758 | 32.241 | 17.453 | 31.322 | 1.00 | 28.03 |
| ATOM | 18388 | CG | LEU | 2758 | 33.689 | 17.047 | 31.630 | 1.00 | 29.70 |
| ATOM | 18389 | CD1 | LEU | 2758 | 34.658 | 18.019 | 30.978 | 1.00 | 30.55 |
| ATOM | 18390 | CD2 | LEU | 2758 | 33.895 | 17.011 | 33.135 | 1.00 | 30.83 |
| ATOM | 18391 | C | LEU | 2758 | 30.367 | 17.072 | 29.716 | 1.00 | 26.67 |
| ATOM | 18392 | O | LEU | 2758 | 30.095 | 15.899 | 29.450 | 1.00 | 26.40 |
| ATOM | 18393 | N | PRO | 2759 | 29.412 | 17.993 | 29.908 | 1.00 | 26.39 |
| ATOM | 18394 | CD | PRO | 2759 | 29.559 | 19.394 | 30.346 | 1.00 | 28.12 |
| ATOM | 18395 | CA | PRO | 2759 | 27.995 | 17.626 | 29.795 | 1.00 | 26.06 |
| ATOM | 18396 | CB | PRO | 2759 | 27.269 | 18.895 | 30.250 | 1.00 | 28.18 |
| ATOM | 18397 | CG | PRO | 2759 | 28.250 | 19.993 | 29.929 | 1.00 | 27.99 |
| ATOM | 18398 | C | PRO | 2759 | 27.560 | 17.161 | 28.401 | 1.00 | 25.95 |
| ATOM | 18399 | O | PRO | 2759 | 26.499 | 16.557 | 28.244 | 1.00 | 24.22 |
| ATOM | 18400 | N | VAL | 2760 | 28.378 | 17.435 | 27.389 | 1.00 | 24.76 |
| ATOM | 18401 | CA | VAL | 2760 | 28.045 | 17.037 | 26.022 | 1.00 | 23.60 |
| ATOM | 18402 | CB | VAL | 2760 | 28.999 | 17.697 | 24.999 | 1.00 | 23.62 |
| ATOM | 18403 | CG1 | VAL | 2760 | 28.613 | 17.292 | 23.592 | 1.00 | 23.23 |
| ATOM | 18404 | CG2 | VAL | 2780 | 28.949 | 19.204 | 25.135 | 1.00 | 24.42 |
| ATOM | 18405 | C | VAL | 2760 | 28.120 | 15.528 | 25.853 | 1.00 | 21.28 |
| ATOM | 18406 | O | VAL | 2760 | 29.142 | 14.912 | 26.145 | 1.00 | 21.37 |
| ATOM | 18407 | N | THR | 2761 | 27.041 | 14.914 | 25.381 | 1.00 | 21.58 |
| ATOM | 18408 | CA | THR | 2761 | 27.096 | 13.475 | 25.205 | 1.00 | 22.07 |
| ATOM | 18409 | CB | THR | 2761 | 26.038 | 12.765 | 26.109 | 1.00 | 25.74 |
| ATOM | 18410 | OG1 | THR | 2761 | 25.041 | 13.706 | 26.540 | 1.00 | 27.70 |
| ATOM | 18411 | CG2 | THR | 2761 | 26.713 | 12.205 | 27.362 | 1.00 | 29.26 |
| ATOM | 18412 | C | THR | 2761 | 27.007 | 13.062 | 23.731 | 1.00 | 20.47 |
| ATOM | 18413 | O | THR | 2761 | 26.850 | 13.907 | 22.851 | 1.00 | 18.18 |
| ATOM | 18414 | N | VAL | 2762 | 27.146 | 11.769 | 23.466 | 1.00 | 17.63 |
| ATOM | 18415 | CA | VAL | 2762 | 27.127 | 11.268 | 22.102 | 1.00 | 18.14 |
| ATOM | 18416 | CB | VAL | 2762 | 27.260 | 9.724 | 22.092 | 1.00 | 16.98 |
| ATOM | 18417 | CG1 | VAL | 2762 | 27.180 | 9.186 | 20.676 | 1.00 | 18.56 |
| ATOM | 18418 | CG2 | VAL | 2762 | 28.601 | 9.330 | 22.709 | 1.00 | 17.91 |
| ATOM | 18419 | C | VAL | 2762 | 25.884 | 11.699 | 21.335 | 1.00 | 17.74 |
| ATOM | 18420 | O | VAL | 2762 | 25.986 | 12.166 | 20.204 | 1.00 | 20.33 |
| ATOM | 18421 | N | ALA | 2763 | 24.714 | 11.555 | 21.950 | 1.00 | 16.50 |
| ATOM | 18422 | CA | ALA | 2763 | 23.461 | 11.939 | 21.308 | 1.00 | 16.78 |
| ATOM | 18423 | CB | ALA | 2763 | 22.291 | 11.727 | 22.274 | 1.00 | 17.39 |
| ATOM | 18424 | C | ALA | 2763 | 23.475 | 13.387 | 20.814 | 1.00 | 16.99 |
| ATOM | 18425 | O | ALA | 2763 | 22.967 | 13.679 | 19.732 | 1.00 | 16.49 |
| ATOM | 18426 | N | ASP | 2764 | 24.049 | 14.289 | 21.607 | 1.00 | 16.36 |
| ATOM | 18427 | CA | ASP | 2764 | 24.118 | 15.696 | 21.228 | 1.00 | 17.12 |
| ATOM | 18428 | CB | ASP | 2764 | 24.719 | 16.544 | 22.355 | 1.00 | 16.73 |
| ATOM | 18429 | CG | ASP | 2764 | 23.886 | 16.518 | 23.622 | 1.00 | 18.74 |
| ATOM | 18430 | OD1 | ASP | 2764 | 22.638 | 16.468 | 23.530 | 1.00 | 17.10 |
| ATOM | 18431 | OD2 | ASP | 2764 | 24.482 | 16.570 | 24.718 | 1.00 | 18.91 |
| ATOM | 18432 | C | ASP | 2764 | 24.968 | 15.875 | 19.977 | 1.00 | 16.55 |
| ATOM | 18433 | O | ASP | 2764 | 24.617 | 16.642 | 19.084 | 1.00 | 15.59 |
| ATOM | 18434 | N | ILE | 2765 | 26.094 | 15.171 | 19.923 | 1.00 | 17.15 |
| ATOM | 18435 | CA | ILE | 2765 | 26.991 | 15.261 | 18.774 | 1.00 | 18.11 |
| ATOM | 18436 | CB | ILE | 2765 | 28.270 | 14.399 | 18.985 | 1.00 | 18.63 |
| ATOM | 18437 | CG2 | ILE | 2765 | 29.151 | 14.452 | 17.740 | 1.00 | 19.14 |
| ATOM | 18438 | CG1 | ILE | 2765 | 29.057 | 14.901 | 20.207 | 1.00 | 18.27 |
| ATOM | 18439 | CD1 | ILE | 2765 | 29.657 | 16.300 | 20.041 | 1.00 | 20.11 |
| ATOM | 18440 | C | ILE | 2765 | 26.263 | 14.787 | 17.509 | 1.00 | 18.09 |
| ATOM | 18441 | O | ILE | 2765 | 26.328 | 15.432 | 16.461 | 1.00 | 17.25 |
| ATOM | 18442 | N | ALA | 2766 | 25.563 | 13.661 | 17.615 | 1.00 | 17.05 |
| ATOM | 18443 | CA | ALA | 2766 | 24.831 | 13.106 | 16.478 | 1.00 | 16.59 |
| ATOM | 18444 | CB | ALA | 2766 | 24.201 | 11.765 | 16.858 | 1.00 | 18.04 |
| ATOM | 18445 | C | ALA | 2766 | 23.749 | 14.060 | 15.983 | 1.00 | 16.20 |
| ATOM | 18446 | O | ALA | 2766 | 23.486 | 14.167 | 14.778 | 1.00 | 14.36 |
| ATOM | 18447 | N | TYR | 2767 | 23.099 | 14.737 | 16.921 | 1.00 | 15.60 |
| ATOM | 18448 | CA | TYR | 2767 | 22.048 | 15.682 | 16.578 | 1.00 | 17.66 |
| ATOM | 18449 | CB | TYR | 2767 | 21.439 | 16.269 | 17.853 | 1.00 | 17.15 |
| ATOM | 18450 | CG | TYR | 2767 | 20.432 | 17.370 | 17.611 | 1.00 | 18.50 |
| ATOM | 18451 | CD1 | TYR | 2767 | 19.212 | 17.107 | 16.998 | 1.00 | 19.60 |
| ATOM | 18452 | CE1 | TYR | 2767 | 18.269 | 18.113 | 16.811 | 1.00 | 19.55 |
| ATOM | 18453 | CD2 | TYR | 2767 | 20.689 | 18.671 | 18.028 | 1.00 | 20.43 |
| ATOM | 18454 | CE2 | TYR | 2767 | 19.754 | 19.684 | 17.845 | 1.00 | 17.99 |
| ATOM | 18455 | CZ | TYR | 2767 | 18.547 | 19.396 | 17.238 | 1.00 | 20.45 |
| ATOM | 18456 | OH | TYR | 2767 | 17.607 | 20.389 | 17.067 | 1.00 | 19.83 |
| ATOM | 18457 | C | TYR | 2767 | 22.612 | 16.812 | 15.715 | 1.00 | 18.01 |
| ATOM | 18458 | O | TYR | 2767 | 22.130 | 17.075 | 14.614 | 1.00 | 19.81 |
| ATOM | 18459 | N | HIS | 2768 | 23.639 | 17.482 | 16.220 | 1.00 | 19.41 |
| ATOM | 18460 | CA | HIS | 2768 | 24.239 | 18.581 | 15.479 | 1.00 | 18.71 |
| ATOM | 18461 | CB | HIS | 2768 | 25.205 | 19.354 | 16.387 | 1.00 | 18.01 |
| ATOM | 18462 | CG | HIS | 2768 | 24.513 | 20.100 | 17.490 | 1.00 | 19.91 |
| ATOM | 18463 | CD2 | HIS | 2768 | 24.343 | 19.799 | 18.801 | 1.00 | 20.46 |
| ATOM | 18464 | ND1 | HIS | 2768 | 23.816 | 21.271 | 17.275 | 1.00 | 19.52 |
| ATOM | 18465 | CE1 | HIS | 2768 | 23.246 | 21.657 | 18.403 | 1.00 | 19.84 |
| ATOM | 18466 | NE2 | HIS | 2768 | 23.549 | 20.782 | 19.344 | 1.00 | 19.32 |
| ATOM | 18467 | C | HIS | 2768 | 24.929 | 18.102 | 14.199 | 1.00 | 17.93 |
| ATOM | 18468 | O | HIS | 2768 | 24.916 | 18.802 | 13.185 | 1.00 | 17.64 |
| ATOM | 18469 | N | THR | 2769 | 25.508 | 16.904 | 14.234 | 1.00 | 17.47 |
| ATOM | 18470 | CA | THR | 2769 | 26.183 | 16.352 | 13.064 | 1.00 | 18.99 |
| ATOM | 18471 | CB | THR | 2769 | 26.810 | 14.970 | 13.381 | 1.00 | 19.01 |
| ATOM | 18472 | OG1 | THR | 2769 | 27.915 | 15.147 | 14.269 | 1.00 | 19.38 |
| ATOM | 18473 | CG2 | THR | 2769 | 27.295 | 14.277 | 12.109 | 1.00 | 19.98 |
| ATOM | 18474 | C | THR | 2769 | 25.207 | 16.216 | 11.894 | 1.00 | 19.84 |
| ATOM | 18475 | O | THR | 2769 | 25.539 | 16.554 | 10.759 | 1.00 | 20.75 |
| ATOM | 18476 | N | ALA | 2770 | 24.005 | 15.720 | 12.173 | 1.00 | 18.34 |
| ATOM | 18477 | CA | ALA | 2770 | 22.997 | 15.563 | 11.137 | 1.00 | 20.30 |
| ATOM | 18478 | CB | ALA | 2770 | 21.784 | 14.809 | 11.686 | 1.00 | 19.96 |
| ATOM | 18479 | C | ALA | 2770 | 22.562 | 16.923 | 10.586 | 1.00 | 19.65 |
| ATOM | 18480 | O | ALA | 2770 | 22.303 | 17.058 | 9.387 | 1.00 | 19.44 |
| ATOM | 18481 | N | ALA | 2771 | 22.481 | 17.931 | 11.453 | 1.00 | 19.52 |
| ATOM | 18482 | CA | ALA | 2771 | 22.083 | 19.265 | 11.010 | 1.00 | 19.17 |
| ATOM | 18483 | CB | ALA | 2771 | 21.887 | 20.199 | 12.213 | 1.00 | 18.60 |
| ATOM | 18484 | C | ALA | 2771 | 23.146 | 19.828 | 10.072 | 1.00 | 19.51 |
| ATOM | 18485 | O | ALA | 2771 | 22.833 | 20.325 | 8.993 | 1.00 | 20.21 |
| ATOM | 18486 | N | VAL | 2772 | 24.405 | 19.740 | 10.481 | 1.00 | 20.13 |
| ATOM | 18487 | CA | VAL | 2772 | 25.498 | 20.238 | 9.653 | 1.00 | 22.46 |
| ATOM | 18488 | CB | VAL | 2772 | 26.864 | 19.996 | 10.332 | 1.00 | 22.59 |
| ATOM | 18489 | CG1 | VAL | 2772 | 27.998 | 20.293 | 9.355 | 1.00 | 22.59 |
| ATOM | 18490 | CG2 | VAL | 2772 | 26.983 | 20.869 | 11.575 | 1.00 | 20.73 |
| ATOM | 18491 | C | VAL | 2772 | 25.485 | 19.544 | 8.291 | 1.00 | 23.49 |
| ATOM | 18492 | O | VAL | 2772 | 25.567 | 20.198 | 7.252 | 1.00 | 23.77 |
| ATOM | 18493 | N | ARG | 2773 | 25.375 | 18.219 | 8.307 | 1.00 | 23.50 |
| ATOM | 18494 | CA | ARG | 2773 | 25.352 | 17.435 | 7.079 | 1.00 | 23.91 |
| ATOM | 18495 | CB | ARG | 2773 | 25.182 | 15.949 | 7.402 | 1.00 | 23.75 |
| ATOM | 18496 | CG | ARG | 2773 | 25.032 | 15.065 | 6.181 | 1.00 | 24.41 |
| ATOM | 18497 | CD | ARG | 2773 | 26.215 | 15.217 | 5.241 | 1.00 | 25.43 |
| ATOM | 18498 | NE | ARG | 2773 | 27.458 | 14.746 | 5.840 | 1.00 | 25.65 |
| ATOM | 18499 | CZ | ARG | 2773 | 28.661 | 14.919 | 5.301 | 1.00 | 27.67 |
| ATOM | 18500 | NH1 | ARG | 2773 | 28.787 | 15.560 | 4.147 | 1.00 | 27.55 |
| ATOM | 18501 | NH2 | ARG | 2773 | 29.742 | 14.449 | 5.912 | 1.00 | 24.82 |
| ATOM | 18502 | C | ARG | 2773 | 24.260 | 17.873 | 6.107 | 1.00 | 23.35 |
| ATOM | 18503 | O | ARG | 2773 | 24.485 | 17.909 | 4.897 | 1.00 | 25.70 |
| ATOM | 18504 | N | ARG | 2774 | 23.081 | 18.198 | 6.626 | 1.00 | 23.07 |
| ATOM | 18505 | CA | ARG | 2774 | 21.979 | 18.639 | 5.777 | 1.00 | 24.60 |
| ATOM | 18506 | CB | ARG | 2774 | 20.698 | 18.814 | 6.597 | 1.00 | 24.60 |
| ATOM | 18507 | CG | ARG | 2774 | 20.163 | 17.532 | 7.217 | 1.00 | 25.89 |
| ATOM | 18508 | CD | ARG | 2774 | 18.759 | 17.730 | 7.780 | 1.00 | 27.00 |
| ATOM | 18509 | NE | ARG | 2774 | 18.237 | 16.502 | 8.381 | 1.00 | 26.44 |
| ATOM | 18510 | CZ | ARG | 2774 | 18.464 | 16.116 | 9.634 | 1.00 | 27.67 |
| ATOM | 18511 | NH1 | ARG | 2774 | 19.203 | 16.862 | 10.445 | 1.00 | 26.75 |
| ATOM | 18512 | NH2 | ARG | 2774 | 17.953 | 14.976 | 10.078 | 1.00 | 29.16 |
| ATOM | 18513 | C | ARG | 2774 | 22.329 | 19.962 | 5.107 | 1.00 | 26.29 |
| ATOM | 18514 | O | ARG | 2774 | 21.917 | 20.225 | 3.975 | 1.00 | 25.64 |
| ATOM | 18515 | N | GLY | 2775 | 23.095 | 20.786 | 5.818 | 1.00 | 26.82 |
| ATOM | 18516 | CA | GLY | 2775 | 23.491 | 22.081 | 5.295 | 1.00 | 29.16 |
| ATOM | 18517 | C | GLY | 2775 | 24.688 | 22.025 | 4.365 | 1.00 | 30.64 |
| ATOM | 18518 | O | GLY | 2775 | 24.869 | 22.911 | 3.528 | 1.00 | 30.02 |
| ATOM | 18519 | N | ALA | 2776 | 25.506 | 20.987 | 4.517 | 1.00 | 30.40 |
| ATOM | 18520 | CA | ALA | 2776 | 26.694 | 20.802 | 3.691 | 1.00 | 32.03 |
| ATOM | 18521 | CB | ALA | 2776 | 27.914 | 21.368 | 4.403 | 1.00 | 31.76 |
| ATOM | 18522 | C | ALA | 2776 | 26.900 | 19.315 | 3.404 | 1.00 | 32.26 |
| ATOM | 18523 | O | ALA | 2776 | 27.754 | 18.670 | 4.006 | 1.00 | 33.25 |
| ATOM | 18524 | N | PRO | 2777 | 26.118 | 18.753 | 2.470 | 1.00 | 32.99 |
| ATOM | 18525 | CD | PRO | 2777 | 25.068 | 19.418 | 1.678 | 1.00 | 33.32 |
| ATOM | 18526 | CA | PRO | 2777 | 26.215 | 17.335 | 2.109 | 1.00 | 33.05 |
| ATOM | 18527 | CB | PRO | 2777 | 25.007 | 17.135 | 1.202 | 1.00 | 34.47 |
| ATOM | 18528 | CG | PRO | 2777 | 24.886 | 18.461 | 0.526 | 1.00 | 34.66 |
| ATOM | 18529 | C | PRO | 2777 | 27.520 | 16.922 | 1.435 | 1.00 | 33.57 |
| ATOM | 18530 | O | PRO | 2777 | 27.824 | 15.734 | 1.349 | 1.00 | 31.96 |
| ATOM | 18531 | N | ASN | 2778 | 28.288 | 17.902 | 0.966 | 1.00 | 33.97 |
| ATOM | 18532 | CA | ASN | 2778 | 29.555 | 17.625 | 0.296 | 1.00 | 35.27 |
| ATOM | 18533 | CB | ASN | 2778 | 29.619 | 18.366 | −1.046 | 1.00 | 36.46 |
| ATOM | 18534 | CG | ASN | 2778 | 28.554 | 17.909 | −2.023 | 1.00 | 38.38 |
| ATOM | 18535 | OD1 | ASN | 2778 | 28.532 | 16.752 | −2.435 | 1.00 | 38.91 |
| ATOM | 18536 | ND2 | ASN | 2778 | 27.661 | 18.822 | −2.399 | 1.00 | 39.92 |
| ATOM | 18537 | C | ASN | 2778 | 30.768 | 18.023 | 1.139 | 1.00 | 34.56 |
| ATOM | 18538 | O | ASN | 2778 | 31.884 | 18.090 | 0.629 | 1.00 | 35.51 |
| ATOM | 18539 | N | CYS | 2779 | 30.558 | 18.284 | 2.425 | 1.00 | 32.91 |
| ATOM | 18540 | CA | CYS | 2779 | 31.664 | 18.683 | 3.285 | 1.00 | 31.31 |
| ATOM | 18541 | CB | CYS | 2779 | 31.202 | 19.708 | 4.335 | 1.00 | 32.14 |
| ATOM | 18542 | SG | CYS | 2779 | 30.456 | 19.001 | 5.868 | 1.00 | 30.62 |
| ATOM | 18543 | C | CYS | 2779 | 32.287 | 17.497 | 4.006 | 1.00 | 31.79 |
| ATOM | 18544 | O | CYS | 2779 | 31.665 | 16.444 | 4.151 | 1.00 | 31.60 |
| ATOM | 18545 | N | LEU | 2780 | 33.531 | 17.673 | 4.436 | 1.00 | 30.11 |
| ATOM | 18546 | CA | LEU | 2780 | 34.231 | 16.647 | 5.191 | 1.00 | 29.59 |
| ATOM | 18547 | CB | LEU | 2780 | 35.744 | 16.787 | 5.018 | 1.00 | 29.72 |
| ATOM | 18548 | CG | LEU | 2780 | 36.593 | 15.754 | 5.768 | 1.00 | 29.00 |
| ATOM | 18549 | CD1 | LEU | 2780 | 36.280 | 14.356 | 5.241 | 1.00 | 29.73 |
| ATOM | 18550 | CD2 | LEU | 2780 | 38.070 | 16.064 | 5.591 | 1.00 | 30.17 |
| ATOM | 18551 | C | LEU | 2780 | 33.841 | 16.958 | 6.632 | 1.00 | 29.34 |
| ATOM | 18552 | O | LEU | 2780 | 34.395 | 17.868 | 7.245 | 1.00 | 28.84 |
| ATOM | 18553 | N | LEU | 2781 | 32.883 | 16.204 | 7.160 | 1.00 | 27.59 |
| ATOM | 18554 | CA | LEU | 2781 | 32.384 | 16.422 | 8.509 | 1.00 | 25.37 |
| ATOM | 18555 | CB | LEU | 2781 | 30.891 | 16.063 | 8.558 | 1.00 | 24.75 |
| ATOM | 18556 | CG | LEU | 2781 | 29.977 | 16.721 | 9.604 | 1.00 | 23.53 |
| ATOM | 18557 | CD1 | LEU | 2781 | 28.525 | 16.303 | 9.359 | 1.00 | 23.06 |
| ATOM | 18558 | CD2 | LEU | 2781 | 30.414 | 16.333 | 10.994 | 1.00 | 22.38 |
| ATOM | 18559 | C | LEU | 2781 | 33.149 | 15.643 | 9.584 | 1.00 | 25.37 |
| ATOM | 18560 | O | LEU | 2781 | 33.200 | 14.415 | 9.563 | 1.00 | 23.46 |
| ATOM | 18561 | N | LEU | 2782 | 33.758 | 16.372 | 10.514 | 1.00 | 26.91 |
| ATOM | 18562 | CA | LEU | 2782 | 34.488 | 15.760 | 11.617 | 1.00 | 26.65 |
| ATOM | 18563 | CB | LEU | 2782 | 35.891 | 16.349 | 11.738 | 1.00 | 29.44 |
| ATOM | 18564 | CG | LEU | 2782 | 36.940 | 15.866 | 10.736 | 1.00 | 31.77 |
| ATOM | 18565 | CD1 | LEU | 2782 | 36.836 | 16.642 | 9.435 | 1.00 | 32.73 |
| ATOM | 18566 | CD2 | LEU | 2782 | 38.316 | 16.046 | 11.354 | 1.00 | 34.44 |
| ATOM | 18567 | C | LEU | 2782 | 33.729 | 16.016 | 12.913 | 1.00 | 26.25 |
| ATOM | 18568 | O | LEU | 2782 | 33.362 | 17.152 | 13.208 | 1.00 | 26.93 |
| ATOM | 18569 | N | ALA | 2783 | 33.495 | 14.966 | 13.692 | 1.00 | 25.81 |
| ATOM | 18570 | CA | ALA | 2783 | 32.775 | 15.116 | 14.953 | 1.00 | 26.30 |
| ATOM | 18571 | CB | ALA | 2783 | 31.397 | 14.481 | 14.843 | 1.00 | 25.81 |
| ATOM | 18572 | C | ALA | 2783 | 33.555 | 14.483 | 16.096 | 1.00 | 23.83 |
| ATOM | 18573 | O | ALA | 2783 | 34.175 | 13.439 | 15.921 | 1.00 | 23.35 |
| ATOM | 18574 | N | ASP | 2784 | 33.532 | 15.112 | 17.267 | 1.00 | 24.55 |
| ATOM | 18575 | CA | ASP | 2784 | 34.258 | 14.565 | 18.409 | 1.00 | 24.44 |
| ATOM | 18576 | CB | ASP | 2784 | 34.644 | 15.649 | 19.420 | 1.00 | 29.43 |
| ATOM | 18577 | CG | ASP | 2784 | 35.301 | 16.852 | 18.787 | 1.00 | 30.77 |
| ATOM | 18578 | OD1 | ASP | 2784 | 35.870 | 16.717 | 17.687 | 1.00 | 34.29 |
| ATOM | 18579 | OD2 | ASP | 2784 | 35.253 | 17.929 | 19.416 | 1.00 | 32.28 |
| ATOM | 18580 | C | ASP | 2784 | 33.445 | 13.549 | 19.184 | 1.00 | 22.89 |
| ATOM | 18581 | O | ASP | 2784 | 32.213 | 13.581 | 19.176 | 1.00 | 22.27 |
| ATOM | 18582 | N | LEU | 2785 | 34.151 | 12.641 | 19.843 | 1.00 | 22.16 |
| ATOM | 18583 | CA | LEU | 2785 | 33.506 | 11.683 | 20.718 | 1.00 | 21.83 |
| ATOM | 18584 | CB | LEU | 2785 | 34.278 | 10.367 | 20.788 | 1.00 | 21.35 |
| ATOM | 18585 | CG | LEU | 2785 | 34.145 | 9.458 | 19.559 | 1.00 | 21.98 |
| ATOM | 18586 | CD1 | LEU | 2785 | 34.687 | 8.077 | 19.896 | 1.00 | 22.46 |
| ATOM | 18587 | CD2 | LEU | 2785 | 32.680 | 9.356 | 19.147 | 1.00 | 22.46 |
| ATOM | 18588 | C | LEU | 2785 | 33.629 | 12.440 | 22.032 | 1.00 | 23.06 |
| ATOM | 18589 | O | LEU | 2785 | 34.738 | 12.760 | 22.472 | 1.00 | 25.15 |
| ATOM | 18590 | N | PRO | 2786 | 32.495 | 12.771 | 22.658 | 1.00 | 22.63 |
| ATOM | 18591 | CD | PRO | 2786 | 31.134 | 12.348 | 22.285 | 1.00 | 22.73 |
| ATOM | 18592 | CA | PRO | 2786 | 32.483 | 13.509 | 23.925 | 1.00 | 22.23 |
| ATOM | 18593 | CB | PRO | 2786 | 30.993 | 13.690 | 24.200 | 1.00 | 22.61 |
| ATOM | 18594 | CG | PRO | 2786 | 30.400 | 12.444 | 23.604 | 1.00 | 24.03 |
| ATOM | 18595 | C | PRO | 2786 | 33.201 | 12.840 | 25.084 | 1.00 | 22.35 |
| ATOM | 18596 | O | PRO | 2786 | 33.774 | 11.761 | 24.945 | 1.00 | 23.47 |
| ATOM | 18597 | N | PHE | 2787 | 33.157 | 13.511 | 26.232 | 1.00 | 23.08 |
| ATOM | 18598 | CA | PHE | 2787 | 33.771 | 13.036 | 27.464 | 1.00 | 21.93 |
| ATOM | 18599 | CB | PHE | 2787 | 33.378 | 13.979 | 28.609 | 1.00 | 24.24 |
| ATOM | 18600 | CG | PHE | 2787 | 33.721 | 13.461 | 29.974 | 1.00 | 25.46 |
| ATOM | 18601 | CD1 | PHE | 2787 | 35.047 | 13.282 | 30.355 | 1.00 | 28.52 |
| ATOM | 18602 | CD2 | PHE | 2787 | 32.714 | 13.151 | 30.885 | 1.00 | 29.32 |
| ATOM | 18603 | CE1 | PHE | 2787 | 35.368 | 12.802 | 31.624 | 1.00 | 29.53 |
| ATOM | 18604 | CE2 | PHE | 2787 | 33.023 | 12.671 | 32.157 | 1.00 | 30.35 |
| ATOM | 18605 | CZ | PHE | 2787 | 34.356 | 12.496 | 32.529 | 1.00 | 31.34 |
| ATOM | 18606 | C | PHE | 2787 | 33.359 | 11.598 | 27.800 | 1.00 | 20.61 |
| ATOM | 18607 | O | PHE | 2787 | 32.174 | 11.260 | 27.788 | 1.00 | 21.01 |
| ATOM | 18608 | N | MET | 2788 | 34.354 | 10.766 | 28.091 | 1.00 | 17.86 |
| ATOM | 18609 | CA | MET | 2788 | 34.156 | 9.364 | 28.452 | 1.00 | 20.41 |
| ATOM | 18610 | CB | MET | 2788 | 33.417 | 9.269 | 29.793 | 1.00 | 21.52 |
| ATOM | 18611 | CG | MET | 2788 | 33.645 | 7.961 | 30.546 | 1.00 | 24.11 |
| ATOM | 18612 | SD | MET | 2788 | 35.397 | 7.699 | 30.917 | 1.00 | 25.02 |
| ATOM | 18613 | CE | MET | 2788 | 35.761 | 9.174 | 31.844 | 1.00 | 27.40 |
| ATOM | 18614 | C | MET | 2788 | 33.405 | 8.555 | 27.396 | 1.00 | 19.70 |
| ATOM | 18615 | O | MET | 2788 | 32.811 | 7.516 | 27.705 | 1.00 | 22.61 |
| ATOM | 18616 | N | ALA | 2789 | 33.430 | 9.020 | 26.152 | 1.00 | 19.87 |
| ATOM | 18617 | CA | ALA | 2789 | 32.736 | 8.321 | 25.069 | 1.00 | 18.87 |
| ATOM | 18618 | CB | ALA | 2789 | 32.236 | 9.326 | 24.036 | 1.00 | 19.03 |
| ATOM | 18619 | C | ALA | 2789 | 33.627 | 7.287 | 24.400 | 1.00 | 19.61 |
| ATOM | 18620 | O | ALA | 2789 | 33.190 | 6.566 | 23.502 | 1.00 | 18.35 |
| ATOM | 18621 | N | TYR | 2790 | 34.880 | 7.213 | 24.832 | 1.00 | 19.23 |
| ATOM | 18622 | CA | TYR | 2790 | 35.813 | 6.250 | 24.257 | 1.00 | 20.39 |
| ATOM | 18623 | CB | TYR | 2790 | 36.632 | 6.911 | 23.146 | 1.00 | 21.61 |
| ATOM | 18624 | CG | TYR | 2790 | 37.208 | 8.255 | 23.518 | 1.00 | 21.98 |
| ATOM | 18625 | CD1 | TYR | 2790 | 38.509 | 8.373 | 24.012 | 1.00 | 24.51 |
| ATOM | 18626 | CE1 | TYR | 2790 | 39.039 | 9.625 | 24.362 | 1.00 | 26.09 |
| ATOM | 18627 | CD2 | TYR | 2790 | 36.445 | 9.413 | 23.385 | 1.00 | 22.55 |
| ATOM | 18628 | CE2 | TYR | 2790 | 36.958 | 10.661 | 23.731 | 1.00 | 25.20 |
| ATOM | 18629 | CZ | TYR | 2790 | 38.252 | 10.760 | 24.216 | 1.00 | 26.07 |
| ATOM | 18630 | OH | TYR | 2790 | 38.752 | 12.006 | 24.538 | 1.00 | 28.26 |
| ATOM | 18631 | C | TYR | 2790 | 36.720 | 5.685 | 25.331 | 1.00 | 21.20 |
| ATOM | 18632 | O | TYR | 2790 | 37.905 | 5.452 | 25.099 | 1.00 | 20.79 |
| ATOM | 18633 | N | ALA | 2791 | 36.136 | 5.443 | 26.501 | 1.00 | 20.62 |
| ATOM | 18634 | CA | ALA | 2791 | 36.863 | 4.902 | 27.649 | 1.00 | 22.09 |
| ATOM | 18635 | CB | ALA | 2791 | 35.957 | 4.894 | 28.876 | 1.00 | 23.98 |
| ATOM | 18636 | C | ALA | 2791 | 37.401 | 3.496 | 27.383 | 1.00 | 21.97 |
| ATOM | 18637 | O | ALA | 2791 | 38.381 | 3.075 | 27.994 | 1.00 | 22.52 |
| ATOM | 18638 | N | THR | 2792 | 36.740 | 2.766 | 26.491 | 1.00 | 20.00 |
| ATOM | 18639 | CA | THR | 2792 | 37.186 | 1.431 | 26.123 | 1.00 | 20.70 |
| ATOM | 18640 | CB | THR | 2792 | 36.348 | 0.313 | 26.786 | 1.00 | 21.59 |
| ATOM | 18641 | OG1 | THR | 2792 | 35.030 | 0.310 | 26.220 | 1.00 | 23.40 |
| ATOM | 18642 | CG2 | THR | 2792 | 36.250 | 0.532 | 28.297 | 1.00 | 20.98 |
| ATOM | 18643 | C | THR | 2792 | 36.999 | 1.325 | 24.616 | 1.00 | 21.35 |
| ATOM | 18644 | O | THR | 2792 | 36.128 | 1.980 | 24.052 | 1.00 | 19.07 |
| ATOM | 18645 | N | PRO | 2793 | 37.824 | 0.510 | 23.945 | 1.00 | 21.50 |
| ATOM | 18646 | CD | PRO | 2793 | 38.961 | −0.267 | 24.471 | 1.00 | 22.59 |
| ATOM | 18647 | CA | PRO | 2793 | 37.711 | 0.348 | 22.494 | 1.00 | 22.71 |
| ATOM | 18648 | CB | PRO | 2793 | 38.738 | −0.737 | 22.197 | 1.00 | 23.86 |
| ATOM | 18649 | CG | PRO | 2793 | 39.806 | −0.458 | 23.233 | 1.00 | 24.23 |
| ATOM | 18650 | C | PRO | 2793 | 36.290 | −0.051 | 22.097 | 1.00 | 21.79 |
| ATOM | 18651 | O | PRO | 2793 | 35.740 | 0.462 | 21.124 | 1.00 | 20.91 |
| ATOM | 18652 | N | GLU | 2794 | 35.694 | −0.954 | 22.867 | 1.00 | 22.08 |
| ATOM | 18653 | CA | GLU | 2794 | 34.341 | −1.412 | 22.588 | 1.00 | 22.74 |
| ATOM | 18654 | CB | GLU | 2794 | 33.932 | −2.482 | 23.603 | 1.00 | 26.70 |
| ATOM | 18655 | CG | GLU | 2794 | 32.778 | −3.362 | 23.157 | 1.00 | 32.07 |
| ATOM | 18656 | CD | GLU | 2794 | 32.483 | −4.484 | 24.143 | 1.00 | 37.26 |
| ATOM | 18657 | OE1 | GLU | 2794 | 32.076 | −4.182 | 25.291 | 1.00 | 38.96 |
| ATOM | 18658 | OE2 | GLU | 2794 | 32.659 | −5.669 | 23.771 | 1.00 | 39.39 |
| ATOM | 18659 | C | GLU | 2794 | 33.367 | −0.231 | 22.628 | 1.00 | 21.49 |
| ATOM | 18660 | O | GLU | 2794 | 32.536 | −0.073 | 21.734 | 1.00 | 18.66 |
| ATOM | 18661 | N | GLN | 2795 | 33.465 | 0.609 | 23.652 | 1.00 | 19.76 |
| ATOM | 18662 | CA | GLN | 2795 | 32.574 | 1.758 | 23.728 | 1.00 | 19.97 |
| ATOM | 18663 | CB | GLN | 2795 | 32.708 | 2.444 | 25.086 | 1.00 | 23.38 |
| ATOM | 18664 | CG | GLN | 2795 | 32.276 | 1.563 | 26.239 | 1.00 | 30.06 |
| ATOM | 18665 | CD | GLN | 2795 | 32.302 | 2.296 | 27.555 | 1.00 | 33.76 |
| ATOM | 18666 | OE1 | GLN | 2795 | 31.557 | 3.257 | 27.755 | 1.00 | 37.23 |
| ATOM | 18667 | NE2 | GLN | 2795 | 33.164 | 1.854 | 28.463 | 1.00 | 37.36 |
| ATOM | 18668 | C | GLN | 2795 | 32.883 | 2.738 | 22.602 | 1.00 | 18.75 |
| ATOM | 18669 | O | GLN | 2795 | 31.979 | 3.379 | 22.055 | 1.00 | 16.21 |
| ATOM | 18670 | N | ALA | 2796 | 34.160 | 2.853 | 22.251 | 1.00 | 17.68 |
| ATOM | 18671 | CA | ALA | 2796 | 34.561 | 3.740 | 21.161 | 1.00 | 16.76 |
| ATOM | 18672 | CB | ALA | 2796 | 36.077 | 3.731 | 21.019 | 1.00 | 17.78 |
| ATOM | 18673 | C | ALA | 2796 | 33.909 | 3.306 | 19.844 | 1.00 | 15.71 |
| ATOM | 18674 | O | ALA | 2796 | 33.408 | 4.146 | 19.097 | 1.00 | 13.16 |
| ATOM | 18675 | N | PHE | 2797 | 33.897 | 2.002 | 19.567 | 1.00 | 16.19 |
| ATOM | 18676 | CA | PHE | 2797 | 33.302 | 1.508 | 18.319 | 1.00 | 16.66 |
| ATOM | 18677 | CB | PHE | 2797 | 33.374 | −0.023 | 18.233 | 1.00 | 15.73 |
| ATOM | 18678 | CG | PHE | 2797 | 34.749 | −0.596 | 18.450 | 1.00 | 19.24 |
| ATOM | 18679 | CD1 | PHE | 2797 | 35.882 | 0.081 | 18.013 | 1.00 | 17.94 |
| ATOM | 18680 | CD2 | PHE | 2797 | 34.907 | −1.823 | 19.088 | 1.00 | 17.33 |
| ATOM | 18681 | CE1 | PHE | 2797 | 37.153 | −0.453 | 18.212 | 1.00 | 20.94 |
| ATOM | 18682 | CE2 | PHE | 2797 | 36.175 | −2.369 | 19.293 | 1.00 | 20.14 |
| ATOM | 18683 | CZ | PHE | 2797 | 37.299 | −1.678 | 18.853 | 1.00 | 18.91 |
| ATOM | 18684 | C | PHE | 2797 | 31.840 | 1.925 | 18.221 | 1.00 | 15.33 |
| ATOM | 18685 | O | PHE | 2797 | 31.398 | 2.438 | 17.197 | 1.00 | 14.05 |
| ATOM | 18686 | N | GLU | 2798 | 31.105 | 1.688 | 19.305 | 1.00 | 17.31 |
| ATOM | 18687 | CA | GLU | 2798 | 29.687 | 2.015 | 19.392 | 1.00 | 18.59 |
| ATOM | 18688 | CB | GLU | 2798 | 29.139 | 1.537 | 20.741 | 1.00 | 23.40 |
| ATOM | 18689 | CG | GLU | 2798 | 27.645 | 1.743 | 20.953 | 1.00 | 27.48 |
| ATOM | 18690 | CD | GLU | 2798 | 26.801 | 0.925 | 19.996 | 1.00 | 31.32 |
| ATOM | 18691 | OE1 | GLU | 2798 | 27.266 | −0.155 | 19.566 | 1.00 | 34.20 |
| ATOM | 18692 | OE2 | GLU | 2798 | 25.667 | 1.353 | 19.685 | 1.00 | 34.45 |
| ATOM | 18693 | C | GLU | 2798 | 29.379 | 3.507 | 19.220 | 1.00 | 17.82 |
| ATOM | 18694 | O | GLU | 2798 | 28.510 | 3.882 | 18.432 | 1.00 | 16.21 |
| ATOM | 18695 | N | ASN | 2799 | 30.082 | 4.363 | 19.956 | 1.00 | 17.26 |
| ATOM | 18696 | CA | ASN | 2799 | 29.808 | 5.790 | 19.863 | 1.00 | 16.33 |
| ATOM | 18697 | CB | ASN | 2799 | 30.402 | 6.521 | 21.069 | 1.00 | 17.15 |
| ATOM | 18698 | CG | ASN | 2799 | 29.710 | 6.125 | 22.369 | 1.00 | 20.67 |
| ATOM | 18699 | OD1 | ASN | 2799 | 28.495 | 5.923 | 22.388 | 1.00 | 17.99 |
| ATOM | 18700 | ND2 | ASN | 2799 | 30.474 | 6.017 | 23.455 | 1.00 | 17.98 |
| ATOM | 18701 | C | ASN | 2799 | 30.267 | 6.411 | 18.553 | 1.00 | 15.61 |
| ATOM | 18702 | O | ASN | 2799 | 29.615 | 7.313 | 18.031 | 1.00 | 15.62 |
| ATOM | 18703 | N | ALA | 2800 | 31.379 | 5.915 | 18.019 | 1.00 | 16.20 |
| ATOM | 18704 | CA | ALA | 2800 | 31.891 | 6.395 | 16.748 | 1.00 | 16.55 |
| ATOM | 18705 | CB | ALA | 2800 | 33.233 | 5.752 | 16.443 | 1.00 | 14.64 |
| ATOM | 18706 | C | ALA | 2800 | 30.885 | 6.050 | 15.653 | 1.00 | 16.73 |
| ATOM | 18707 | O | ALA | 2800 | 30.570 | 6.877 | 14.792 | 1.00 | 17.09 |
| ATOM | 18708 | N | ALA | 2801 | 30.375 | 4.825 | 15.678 | 1.00 | 16.50 |
| ATOM | 18709 | CA | ALA | 2801 | 29.413 | 4.412 | 14.663 | 1.00 | 16.96 |
| ATOM | 18710 | CB | ALA | 2801 | 29.023 | 2.953 | 14.865 | 1.00 | 17.94 |
| ATOM | 18711 | C | ALA | 2801 | 28.180 | 5.306 | 14.726 | 1.00 | 15.19 |
| ATOM | 18712 | O | ALA | 2801 | 27.618 | 5.662 | 13.696 | 1.00 | 17.52 |
| ATOM | 18713 | N | THR | 2802 | 27.765 | 5.679 | 15.934 | 1.00 | 16.31 |
| ATOM | 18714 | CA | THR | 2802 | 26.596 | 6.528 | 16.082 | 1.00 | 15.68 |
| ATOM | 18715 | CB | THR | 2802 | 26.265 | 6.808 | 17.572 | 1.00 | 16.35 |
| ATOM | 18716 | OG1 | THR | 2802 | 25.995 | 5.577 | 18.252 | 1.00 | 14.35 |
| ATOM | 18717 | CG2 | THR | 2802 | 25.041 | 7.713 | 17.681 | 1.00 | 14.96 |
| ATOM | 18718 | C | THR | 2802 | 26.778 | 7.869 | 15.369 | 1.00 | 16.13 |
| ATOM | 18719 | O | THR | 2802 | 25.895 | 8.318 | 14.636 | 1.00 | 17.22 |
| ATOM | 18720 | N | VAL | 2803 | 27.921 | 8.509 | 15.580 | 1.00 | 16.63 |
| ATOM | 18721 | CA | VAL | 2803 | 28.186 | 9.798 | 14.956 | 1.00 | 17.54 |
| ATOM | 18722 | CB | VAL | 2803 | 29.380 | 10.502 | 15.644 | 1.00 | 18.75 |
| ATOM | 18723 | CG1 | VAL | 2803 | 29.719 | 11.786 | 14.915 | 1.00 | 23.96 |
| ATOM | 18724 | CG2 | VAL | 2803 | 29.023 | 10.813 | 17.098 | 1.00 | 20.56 |
| ATOM | 18725 | C | VAL | 2803 | 28.449 | 9.669 | 13.458 | 1.00 | 16.45 |
| ATOM | 18726 | O | VAL | 2803 | 28.126 | 10.574 | 12.684 | 1.00 | 18.21 |
| ATOM | 18727 | N | MET | 2804 | 29.029 | 8.547 | 13.051 | 1.00 | 16.81 |
| ATOM | 18728 | CA | MET | 2804 | 29.303 | 8.304 | 11.639 | 1.00 | 17.14 |
| ATOM | 18729 | CB | MET | 2804 | 30.229 | 7.093 | 11.480 | 1.00 | 18.19 |
| ATOM | 18730 | CG | MET | 2804 | 31.654 | 7.295 | 11.995 | 1.00 | 22.52 |
| ATOM | 18731 | SD | MET | 2804 | 32.594 | 8.461 | 10.990 | 1.00 | 24.27 |
| ATOM | 18732 | CE | MET | 2804 | 32.659 | 7.556 | 9.422 | 1.00 | 20.51 |
| ATOM | 18733 | C | MET | 2804 | 27.984 | 8.063 | 10.903 | 1.00 | 16.01 |
| ATOM | 18734 | O | MET | 2804 | 27.761 | 8.618 | 9.824 | 1.00 | 15.88 |
| ATOM | 18735 | N | ARG | 2805 | 27.101 | 7.248 | 11.481 | 1.00 | 15.73 |
| ATOM | 18736 | CA | ARG | 2805 | 25.822 | 6.986 | 10.831 | 1.00 | 15.34 |
| ATOM | 18737 | CB | ARG | 2805 | 25.011 | 5.925 | 11.596 | 1.00 | 13.01 |
| ATOM | 18738 | CG | ARG | 2805 | 25.647 | 4.539 | 11.632 | 1.00 | 16.75 |
| ATOM | 18739 | CD | ARG | 2805 | 24.645 | 3.460 | 12.060 | 1.00 | 13.35 |
| ATOM | 18740 | NE | ARG | 2805 | 25.314 | 2.212 | 12.432 | 1.00 | 18.35 |
| ATOM | 18741 | CZ | ARG | 2805 | 25.762 | 1.941 | 13.652 | 1.00 | 16.28 |
| ATOM | 18742 | NH1 | ARG | 2805 | 25.609 | 2.829 | 14.630 | 1.00 | 18.45 |
| ATOM | 18743 | NH2 | ARG | 2805 | 26.369 | 0.789 | 13.894 | 1.00 | 17.59 |
| ATOM | 18744 | C | ARG | 2805 | 24.999 | 8.268 | 10.718 | 1.00 | 15.55 |
| ATOM | 18745 | O | ARG | 2805 | 24.179 | 8.404 | 9.817 | 1.00 | 14.99 |
| ATOM | 18746 | N | ALA | 2806 | 25.232 | 9.207 | 11.633 | 1.00 | 16.80 |
| ATOM | 18747 | CA | ALA | 2806 | 24.502 | 10.473 | 11.639 | 1.00 | 16.91 |
| ATOM | 18748 | CB | ALA | 2806 | 24.548 | 11.095 | 13.030 | 1.00 | 16.95 |
| ATOM | 18749 | C | ALA | 2806 | 24.997 | 11.483 | 10.602 | 1.00 | 18.90 |
| ATOM | 18750 | O | ALA | 2806 | 24.425 | 12.567 | 10.466 | 1.00 | 16.85 |
| ATOM | 18751 | N | GLY | 2807 | 26.062 | 11.144 | 9.880 | 1.00 | 17.86 |
| ATOM | 18752 | CA | GLY | 2807 | 26.555 | 12.055 | 8.862 | 1.00 | 20.83 |
| ATOM | 18753 | C | GLY | 2807 | 28.042 | 12.344 | 8.866 | 1.00 | 19.96 |
| ATOM | 18754 | O | GLY | 2807 | 28.583 | 12.841 | 7.873 | 1.00 | 20.33 |
| ATOM | 18755 | N | ALA | 2808 | 28.706 | 12.039 | 9.976 | 1.00 | 20.84 |
| ATOM | 18756 | CA | ALA | 2808 | 30.141 | 12.279 | 10.094 | 1.00 | 20.51 |
| ATOM | 18757 | CB | ALA | 2808 | 30.596 | 12.030 | 11.532 | 1.00 | 21.43 |
| ATOM | 18758 | C | ALA | 2808 | 30.983 | 11.433 | 9.140 | 1.00 | 22.32 |
| ATOM | 18759 | O | ALA | 2808 | 30.583 | 10.340 | 8.734 | 1.00 | 22.72 |
| ATOM | 18760 | N | ASN | 2809 | 32.154 | 11.954 | 8.783 | 1.00 | 21.83 |
| ATOM | 18761 | CA | ASN | 2809 | 33.076 | 11.252 | 7.895 | 1.00 | 23.00 |
| ATOM | 18762 | CB | ASN | 2809 | 33.563 | 12.161 | 6.756 | 1.00 | 23.04 |
| ATOM | 18763 | CG | ASN | 2809 | 32.454 | 12.563 | 5.813 | 1.00 | 22.97 |
| ATOM | 18764 | OD1 | ASN | 2809 | 31.720 | 11.715 | 5.303 | 1.00 | 26.20 |
| ATOM | 18765 | ND2 | ASN | 2809 | 32.329 | 13.856 | 5.567 | 1.00 | 24.82 |
| ATOM | 18766 | C | ASN | 2809 | 34.281 | 10.828 | 8.713 | 1.00 | 23.50 |
| ATOM | 18767 | O | ASN | 2809 | 34.991 | 9.885 | 8.365 | 1.00 | 22.98 |
| ATOM | 18768 | N | MET | 2810 | 34.503 | 11.531 | 9.815 | 1.00 | 23.62 |
| ATOM | 18769 | CA | MET | 2810 | 35.642 | 11.247 | 10.669 | 1.00 | 23.93 |
| ATOM | 18770 | CB | MET | 2810 | 36.853 | 12.052 | 10.181 | 1.00 | 25.09 |
| ATOM | 18771 | CG | MET | 2810 | 38.126 | 11.879 | 10.995 | 1.00 | 24.78 |
| ATOM | 18772 | SD | MET | 2810 | 39.481 | 12.847 | 10.255 | 1.00 | 28.00 |
| ATOM | 18773 | CE | MET | 2810 | 40.353 | 11.582 | 9.303 | 1.00 | 27.11 |
| ATOM | 18774 | C | MET | 2810 | 35.318 | 11.602 | 12.112 | 1.00 | 22.92 |
| ATOM | 18775 | O | MET | 2810 | 34.490 | 12.469 | 12.377 | 1.00 | 22.76 |
| ATOM | 18776 | N | VAL | 2811 | 35.972 | 10.912 | 13.037 | 1.00 | 21.54 |
| ATOM | 18777 | CA | VAL | 2811 | 35.767 | 11.144 | 14.452 | 1.00 | 22.27 |
| ATOM | 18778 | CB | VAL | 2811 | 35.345 | 9.840 | 15.156 | 1.00 | 22.64 |
| ATOM | 18779 | CG1 | VAL | 2811 | 35.310 | 10.042 | 16.639 | 1.00 | 27.23 |
| ATOM | 18780 | CG2 | VAL | 2811 | 33.972 | 9.399 | 14.652 | 1.00 | 23.31 |
| ATOM | 18781 | C | VAL | 2811 | 37.052 | 11.665 | 15.092 | 1.00 | 21.34 |
| ATOM | 18782 | O | VAL | 2811 | 38.151 | 11.238 | 14.729 | 1.00 | 20.30 |
| ATOM | 18783 | N | LYS | 2812 | 36.912 | 12.593 | 16.037 | 1.00 | 21.70 |
| ATOM | 18784 | CA | LYS | 2812 | 38.066 | 13.153 | 16.737 | 1.00 | 21.55 |
| ATOM | 18785 | CB | LYS | 2812 | 38.114 | 14.679 | 16.577 | 1.00 | 20.79 |
| ATOM | 18786 | CG | LYS | 2812 | 39.283 | 15.330 | 17.324 | 1.00 | 22.48 |
| ATOM | 18787 | CD | LYS | 2812 | 39.567 | 16.749 | 16.830 | 1.00 | 21.07 |
| ATOM | 18788 | CE | LYS | 2812 | 38.508 | 17.730 | 17.295 | 1.00 | 21.59 |
| ATOM | 18789 | NZ | LYS | 2812 | 38.526 | 17.887 | 18.777 | 1.00 | 19.95 |
| ATOM | 18790 | C | LYS | 2812 | 38.027 | 12.803 | 18.216 | 1.00 | 21.09 |
| ATOM | 18791 | O | LYS | 2812 | 37.000 | 12.967 | 18.873 | 1.00 | 20.83 |
| ATOM | 18792 | N | ILE | 2813 | 39.151 | 12.311 | 18.729 | 1.00 | 23.63 |
| ATOM | 18793 | CA | ILE | 2813 | 39.271 | 11.941 | 20.138 | 1.00 | 25.12 |
| ATOM | 18794 | CB | ILE | 2813 | 39.341 | 10.403 | 20.318 | 1.00 | 26.14 |
| ATOM | 18795 | CG2 | ILE | 2813 | 38.018 | 9.763 | 19.878 | 1.00 | 25.11 |
| ATOM | 18796 | CG1 | ILE | 2813 | 40.495 | 9.829 | 19.499 | 1.00 | 27.05 |
| ATOM | 18797 | CD1 | ILE | 2813 | 40.733 | 8.341 | 19.742 | 1.00 | 28.78 |
| ATOM | 18798 | C | ILE | 2813 | 40.542 | 12.560 | 20.735 | 1.00 | 27.72 |
| ATOM | 18799 | O | ILE | 2813 | 41.580 | 12.607 | 20.078 | 1.00 | 26.81 |
| ATOM | 18800 | N | GLU | 2814 | 40.454 | 13.028 | 21.978 | 1.00 | 29.43 |
| ATOM | 18801 | CA | GLU | 2814 | 41.591 | 13.651 | 22.650 | 1.00 | 32.04 |
| ATOM | 18802 | CB | GLU | 2814 | 41.106 | 14.813 | 23.522 | 1.00 | 34.21 |
| ATOM | 18803 | CG | GLU | 2814 | 40.144 | 15.750 | 22.806 | 1.00 | 39.30 |
| ATOM | 18804 | CD | GLU | 2814 | 39.764 | 16.966 | 23.636 | 1.00 | 41.47 |
| ATOM | 18805 | OE1 | GLU | 2814 | 39.370 | 16.798 | 24.812 | 1.00 | 41.55 |
| ATOM | 18806 | OE2 | GLU | 2814 | 39.852 | 18.093 | 23.105 | 1.00 | 44.11 |
| ATOM | 18807 | C | GLU | 2814 | 42.364 | 12.655 | 23.509 | 1.00 | 31.92 |
| ATOM | 18808 | O | GLU | 2814 | 41.779 | 11.914 | 24.296 | 1.00 | 33.22 |
| ATOM | 18809 | N | GLY | 2815 | 43.684 | 12.641 | 23.354 | 1.00 | 30.77 |
| ATOM | 18810 | CA | GLY | 2815 | 44.506 | 11.729 | 24.126 | 1.00 | 30.34 |
| ATOM | 18811 | C | GLY | 2815 | 45.801 | 11.392 | 23.415 | 1.00 | 31.29 |
| ATOM | 18812 | O | GLY | 2815 | 45.975 | 11.705 | 22.234 | 1.00 | 30.87 |
| ATOM | 18813 | N | GLY | 2816 | 46.715 | 10.748 | 24.134 | 1.00 | 31.99 |
| ATOM | 18814 | CA | GLY | 2816 | 47.992 | 10.387 | 23.547 | 1.00 | 33.07 |
| ATOM | 18815 | C | GLY | 2816 | 48.172 | 8.903 | 23.294 | 1.00 | 34.28 |
| ATOM | 18816 | O | GLY | 2816 | 47.325 | 8.261 | 22.675 | 1.00 | 34.33 |
| ATOM | 18817 | N | GLU | 2817 | 49.285 | 8.364 | 23.783 | 1.00 | 34.03 |
| ATOM | 18818 | CA | GLU | 2817 | 49.631 | 6.956 | 23.615 | 1.00 | 34.66 |
| ATOM | 18819 | CB | GLU | 2817 | 50.925 | 6.647 | 24.376 | 1.00 | 37.31 |
| ATOM | 18820 | CG | GLU | 2817 | 52.196 | 7.025 | 23.634 | 1.00 | 40.46 |
| ATOM | 18821 | CD | GLU | 2817 | 52.586 | 5.990 | 22.591 | 1.00 | 42.88 |
| ATOM | 18822 | OE1 | GLU | 2817 | 51.711 | 5.590 | 21.793 | 1.00 | 44.37 |
| ATOM | 18823 | OE2 | GLU | 2817 | 53.767 | 5.582 | 22.567 | 1.00 | 42.78 |
| ATOM | 18824 | C | GLU | 2817 | 48.569 | 5.946 | 24.032 | 1.00 | 33.69 |
| ATOM | 18825 | O | GLU | 2817 | 48.395 | 4.923 | 23.369 | 1.00 | 34.29 |
| ATOM | 18826 | N | TRP | 2818 | 47.866 | 6.220 | 25.127 | 1.00 | 32.46 |
| ATOM | 18827 | CA | TRP | 2818 | 46.855 | 5.290 | 25.613 | 1.00 | 30.43 |
| ATOM | 18828 | CB | TRP | 2818 | 46.254 | 5.779 | 26.942 | 1.00 | 30.40 |
| ATOM | 18829 | CG | TRP | 2818 | 45.293 | 6.930 | 26.822 | 1.00 | 29.06 |
| ATOM | 18830 | CD2 | TRP | 2818 | 43.862 | 6.850 | 26.831 | 1.00 | 28.87 |
| ATOM | 18831 | CE2 | TRP | 2818 | 43.367 | 8.166 | 26.698 | 1.00 | 28.69 |
| ATOM | 18832 | CE3 | TRP | 2818 | 42.950 | 5.791 | 26.941 | 1.00 | 27.56 |
| ATOM | 18833 | CD1 | TRP | 2818 | 45.601 | 8.252 | 26.684 | 1.00 | 30.79 |
| ATOM | 18834 | NE1 | TRP | 2818 | 44.448 | 9.005 | 26.609 | 1.00 | 30.25 |
| ATOM | 18835 | CZ2 | TRP | 2818 | 41.997 | 8.451 | 26.667 | 1.00 | 27.27 |
| ATOM | 18836 | CZ3 | TRP | 2818 | 41.590 | 6.074 | 26.910 | 1.00 | 27.33 |
| ATOM | 18837 | CH2 | TRP | 2818 | 41.127 | 7.399 | 26.776 | 1.00 | 27.02 |
| ATOM | 18838 | C | TRP | 2818 | 45.737 | 5.048 | 24.604 | 1.00 | 28.89 |
| ATOM | 18839 | O | TRP | 2818 | 44.964 | 4.105 | 24.741 | 1.00 | 30.31 |
| ATOM | 18840 | N | LEU | 2819 | 45.656 | 5.892 | 23.585 | 1.00 | 27.35 |
| ATOM | 18841 | CA | LEU | 2819 | 44.616 | 5.744 | 22.579 | 1.00 | 26.42 |
| ATOM | 18842 | CB | LEU | 2819 | 44.132 | 7.118 | 22.115 | 1.00 | 26.77 |
| ATOM | 18843 | CG | LEU | 2819 | 43.245 | 7.896 | 23.085 | 1.00 | 28.13 |
| ATOM | 18844 | CD1 | LEU | 2819 | 42.926 | 9.257 | 22.487 | 1.00 | 29.42 |
| ATOM | 18845 | CD2 | LEU | 2819 | 41.969 | 7.121 | 23.358 | 1.00 | 29.42 |
| ATOM | 18846 | C | LEU | 2819 | 45.040 | 4.930 | 21.364 | 1.00 | 25.13 |
| ATOM | 18847 | O | LEU | 2819 | 44.218 | 4.647 | 20.497 | 1.00 | 21.39 |
| ATOM | 18848 | N | VAL | 2820 | 46.315 | 4.550 | 21.297 | 1.00 | 24.43 |
| ATOM | 18849 | CA | VAL | 2820 | 46.814 | 3.783 | 20.155 | 1.00 | 26.56 |
| ATOM | 18850 | CB | VAL | 2820 | 48.258 | 3.281 | 20.395 | 1.00 | 26.68 |
| ATOM | 18851 | CG1 | VAL | 2820 | 48.671 | 2.330 | 19.282 | 1.00 | 27.02 |
| ATOM | 18852 | CG2 | VAL | 2820 | 49.210 | 4.461 | 20.446 | 1.00 | 28.43 |
| ATOM | 18853 | C | VAL | 2820 | 45.942 | 2.587 | 19.774 | 1.00 | 25.78 |
| ATOM | 18854 | O | VAL | 2820 | 45.503 | 2.471 | 18.631 | 1.00 | 27.60 |
| ATOM | 18855 | N | GLU | 2821 | 45.704 | 1.696 | 20.729 | 1.00 | 24.73 |
| ATOM | 18856 | CA | GLU | 2821 | 44.895 | 0.514 | 20.477 | 1.00 | 25.30 |
| ATOM | 18857 | CB | GLU | 2821 | 44.782 | −0.321 | 21.755 | 1.00 | 28.80 |
| ATOM | 18858 | CG | GLU | 2821 | 44.051 | −1.643 | 21.574 | 1.00 | 32.13 |
| ATOM | 18859 | CD | GLU | 2821 | 43.926 | −2.416 | 22.870 | 1.00 | 35.13 |
| ATOM | 18860 | OE1 | GLU | 2821 | 43.340 | −1.871 | 23.830 | 1.00 | 36.98 |
| ATOM | 18861 | OE2 | GLU | 2821 | 44.410 | −3.566 | 22.927 | 1.00 | 36.11 |
| ATOM | 18862 | C | GLU | 2821 | 43.501 | 0.876 | 19.962 | 1.00 | 24.62 |
| ATOM | 18863 | O | GLU | 2821 | 42.979 | 0.236 | 19.046 | 1.00 | 21.97 |
| ATOM | 18864 | N | THR | 2822 | 42.900 | 1.905 | 20.549 | 1.00 | 24.29 |
| ATOM | 18865 | CA | THR | 2822 | 41.568 | 2.336 | 20.141 | 1.00 | 23.99 |
| ATOM | 18866 | CB | THR | 2822 | 41.021 | 3.415 | 21.099 | 1.00 | 24.68 |
| ATOM | 18867 | OG1 | THR | 2822 | 40.929 | 2.868 | 22.422 | 1.00 | 24.15 |
| ATOM | 18868 | CG2 | THR | 2822 | 39.635 | 3.882 | 20.652 | 1.00 | 23.04 |
| ATOM | 18869 | C | THR | 2822 | 41.590 | 2.876 | 18.715 | 1.00 | 24.63 |
| ATOM | 18870 | O | THR | 2822 | 40.671 | 2.631 | 17.927 | 1.00 | 21.47 |
| ATOM | 18871 | N | VAL | 2823 | 42.645 | 3.611 | 18.381 | 1.00 | 23.83 |
| ATOM | 18872 | CA | VAL | 2823 | 42.785 | 4.170 | 17.041 | 1.00 | 23.79 |
| ATOM | 18873 | CB | VAL | 2823 | 44.002 | 5.116 | 16.961 | 1.00 | 25.30 |
| ATOM | 18874 | CG1 | VAL | 2823 | 44.159 | 5.638 | 15.535 | 1.00 | 25.93 |
| ATOM | 18875 | CG2 | VAL | 2823 | 43.816 | 6.276 | 17.928 | 1.00 | 25.50 |
| ATOM | 18876 | C | VAL | 2823 | 42.948 | 3.063 | 16.005 | 1.00 | 23.15 |
| ATOM | 18877 | O | VAL | 2823 | 42.275 | 3.063 | 14.974 | 1.00 | 21.77 |
| ATOM | 18878 | N | GLN | 2824 | 43.839 | 2.117 | 16.296 | 1.00 | 24.00 |
| ATOM | 18879 | CA | GLN | 2824 | 44.106 | 1.000 | 15.395 | 1.00 | 24.92 |
| ATOM | 18880 | CB | GLN | 2824 | 45.156 | 0.068 | 16.005 | 1.00 | 27.02 |
| ATOM | 18881 | CG | GLN | 2824 | 46.476 | 0.737 | 16.333 | 1.00 | 33.50 |
| ATOM | 18882 | CD | GLN | 2824 | 47.494 | −0.229 | 16.908 | 1.00 | 35.79 |
| ATOM | 18883 | OE1 | GLN | 2824 | 47.242 | −0.891 | 17.917 | 1.00 | 38.12 |
| ATOM | 18884 | NE2 | GLN | 2824 | 48.656 | −0.312 | 16.267 | 1.00 | 38.24 |
| ATOM | 18885 | C | GLN | 2824 | 42.840 | 0.207 | 15.101 | 1.00 | 23.85 |
| ATOM | 18886 | O | GLN | 2824 | 42.518 | −0.057 | 13.946 | 1.00 | 25.38 |
| ATOM | 18887 | N | MET | 2825 | 42.126 | −0.167 | 16.155 | 1.00 | 23.02 |
| ATOM | 18888 | CA | MET | 2825 | 40.905 | −0.946 | 16.010 | 1.00 | 22.80 |
| ATOM | 18889 | CB | MET | 2825 | 40.438 | −1.426 | 17.385 | 1.00 | 23.09 |
| ATOM | 18890 | CG | MET | 2825 | 41.414 | −2.396 | 18.028 | 1.00 | 24.85 |
| ATOM | 18891 | SD | MET | 2825 | 40.854 | −3.051 | 19.588 | 1.00 | 26.27 |
| ATOM | 18892 | CE | MET | 2825 | 39.744 | −4.323 | 19.026 | 1.00 | 27.47 |
| ATOM | 18893 | C | MET | 2825 | 39.786 | −0.196 | 15.298 | 1.00 | 20.09 |
| ATOM | 18894 | O | MET | 2825 | 39.024 | −0.793 | 14.543 | 1.00 | 19.10 |
| ATOM | 18895 | N | LEU | 2826 | 39.685 | 1.109 | 15.537 | 1.00 | 20.85 |
| ATOM | 18896 | CA | LEU | 2826 | 38.655 | 1.915 | 14.893 | 1.00 | 22.61 |
| ATOM | 18897 | CB | LEU | 2826 | 38.620 | 3.325 | 15.488 | 1.00 | 20.71 |
| ATOM | 18898 | CG | LEU | 2826 | 37.867 | 3.457 | 16.819 | 1.00 | 23.07 |
| ATOM | 18899 | CD1 | LEU | 2826 | 38.183 | 4.783 | 17.487 | 1.00 | 21.86 |
| ATOM | 18900 | CD2 | LEU | 2826 | 36.373 | 3.333 | 16.548 | 1.00 | 21.56 |
| ATOM | 18901 | C | LEU | 2826 | 38.883 | 1.998 | 13.385 | 1.00 | 24.44 |
| ATOM | 18902 | O | LEU | 2826 | 37.944 | 1.870 | 12.597 | 1.00 | 22.55 |
| ATOM | 18903 | N | THR | 2827 | 40.135 | 2.204 | 12.990 | 1.00 | 26.28 |
| ATOM | 18904 | CA | THR | 2827 | 40.474 | 2.301 | 11.578 | 1.00 | 29.53 |
| ATOM | 18905 | CB | THR | 2827 | 41.988 | 2.511 | 11.384 | 1.00 | 30.21 |
| ATOM | 18906 | OG1 | THR | 2827 | 42.382 | 3.734 | 12.020 | 1.00 | 34.27 |
| ATOM | 18907 | CG2 | THR | 2827 | 42.327 | 2.588 | 9.901 | 1.00 | 33.86 |
| ATOM | 18908 | C | THR | 2827 | 40.034 | 1.060 | 10.795 | 1.00 | 27.89 |
| ATOM | 18909 | O | THR | 2827 | 39.349 | 1.173 | 9.779 | 1.00 | 28.82 |
| ATOM | 18910 | N | GLU | 2828 | 40.416 | −0.121 | 11.268 | 1.00 | 28.30 |
| ATOM | 18911 | CA | GLU | 2828 | 40.041 | −1.350 | 10.580 | 1.00 | 28.55 |
| ATOM | 18912 | CB | GLU | 2828 | 40.775 | −2.548 | 11.179 | 1.00 | 30.32 |
| ATOM | 18913 | CG | GLU | 2828 | 41.107 | −2.404 | 12.638 | 1.00 | 31.98 |
| ATOM | 18914 | CD | GLU | 2828 | 41.836 | −3.614 | 13.180 | 1.00 | 32.11 |
| ATOM | 18915 | OE1 | GLU | 2828 | 42.836 | −4.041 | 12.564 | 1.00 | 32.16 |
| ATOM | 18916 | OE2 | GLU | 2828 | 41.415 | −4.134 | 14.235 | 1.00 | 30.79 |
| ATOM | 18917 | C | GLU | 2828 | 38.540 | −1.588 | 10.592 | 1.00 | 27.06 |
| ATOM | 18918 | O | GLU | 2828 | 38.030 | −2.412 | 9.831 | 1.00 | 27.55 |
| ATOM | 18919 | N | ARG | 2829 | 37.829 | −0.857 | 11.444 | 1.00 | 23.40 |
| ATOM | 18920 | CA | ARG | 2829 | 36.384 | −0.997 | 11.513 | 1.00 | 22.00 |
| ATOM | 18921 | CB | ARG | 2829 | 35.931 | −1.037 | 12.973 | 1.00 | 20.32 |
| ATOM | 18922 | CG | ARG | 2829 | 36.241 | −2.378 | 13.629 | 1.00 | 19.39 |
| ATOM | 18923 | CD | ARG | 2829 | 36.285 | −2.294 | 15.140 | 1.00 | 18.70 |
| ATOM | 18924 | NE | ARG | 2829 | 36.650 | −3.585 | 15.719 | 1.00 | 16.89 |
| ATOM | 18925 | CZ | ARG | 2829 | 37.869 | −4.112 | 15.666 | 1.00 | 17.72 |
| ATOM | 18926 | NH1 | ARG | 2829 | 38.856 | −3.459 | 15.065 | 1.00 | 16.97 |
| ATOM | 18927 | NH2 | ARG | 2829 | 38.102 | −5.303 | 16.201 | 1.00 | 21.33 |
| ATOM | 18928 | C | ARG | 2829 | 35.674 | 0.104 | 10.739 | 1.00 | 20.80 |
| ATOM | 18929 | O | ARG | 2829 | 34.536 | 0.458 | 11.040 | 1.00 | 19.63 |
| ATOM | 18930 | N | ALA | 2830 | 36.368 | 0.642 | 9.737 | 1.00 | 21.90 |
| ATOM | 18931 | CA | ALA | 2830 | 35.825 | 1.680 | 8.856 | 1.00 | 21.97 |
| ATOM | 18932 | CB | ALA | 2830 | 34.474 | 1.223 | 8.305 | 1.00 | 22.04 |
| ATOM | 18933 | C | ALA | 2830 | 35.690 | 3.086 | 9.435 | 1.00 | 22.69 |
| ATOM | 18934 | O | ALA | 2830 | 35.042 | 3.937 | 8.828 | 1.00 | 22.61 |
| ATOM | 18935 | N | VAL | 2831 | 36.297 | 3.341 | 10.589 | 1.00 | 22.14 |
| ATOM | 18936 | CA | VAL | 2831 | 36.194 | 4.662 | 11.194 | 1.00 | 22.55 |
| ATOM | 18937 | CB | VAL | 2831 | 35.679 | 4.570 | 12.651 | 1.00 | 23.25 |
| ATOM | 18938 | CG1 | VAL | 2831 | 35.612 | 5.957 | 13.264 | 1.00 | 22.11 |
| ATOM | 18939 | CG2 | VAL | 2831 | 34.300 | 3.910 | 12.684 | 1.00 | 21.29 |
| ATOM | 18940 | C | VAL | 2831 | 37.520 | 5.427 | 11.207 | 1.00 | 22.41 |
| ATOM | 18941 | O | VAL | 2831 | 38.439 | 5.071 | 11.940 | 1.00 | 21.58 |
| ATOM | 18942 | N | PRO | 2832 | 37.634 | 6.490 | 10.389 | 1.00 | 22.74 |
| ATOM | 18943 | CD | PRO | 2832 | 36.681 | 6.962 | 9.370 | 1.00 | 23.21 |
| ATOM | 18944 | CA | PRO | 2832 | 38.870 | 7.279 | 10.350 | 1.00 | 23.36 |
| ATOM | 18945 | CB | PRO | 2832 | 38.710 | 8.110 | 9.081 | 1.00 | 24.93 |
| ATOM | 18946 | CG | PRO | 2832 | 37.244 | 8.323 | 9.011 | 1.00 | 26.31 |
| ATOM | 18947 | C | PRO | 2832 | 38.961 | 8.129 | 11.611 | 1.00 | 23.38 |
| ATOM | 18948 | O | PRO | 2832 | 37.954 | 8.644 | 12.093 | 1.00 | 20.50 |
| ATOM | 18949 | N | VAL | 2833 | 40.169 | 8.274 | 12.143 | 1.00 | 23.46 |
| ATOM | 18950 | CA | VAL | 2833 | 40.352 | 9.035 | 13.368 | 1.00 | 24.56 |
| ATOM | 18951 | CB | VAL | 2833 | 40.985 | 8.147 | 14.466 | 1.00 | 25.29 |
| ATOM | 18952 | CG1 | VAL | 2833 | 41.061 | 8.909 | 15.778 | 1.00 | 26.10 |
| ATOM | 18953 | CG2 | VAL | 2833 | 40.167 | 6.869 | 14.638 | 1.00 | 26.15 |
| ATOM | 18954 | C | VAL | 2833 | 41.206 | 10.284 | 13.203 | 1.00 | 26.06 |
| ATOM | 18955 | O | VAL | 2833 | 42.156 | 10.307 | 12.420 | 1.00 | 22.96 |
| ATOM | 18956 | N | CYS | 2834 | 40.842 | 11.325 | 13.943 | 1.00 | 26.64 |
| ATOM | 18957 | CA | CYS | 2834 | 41.580 | 12.583 | 13.943 | 1.00 | 27.09 |
| ATOM | 18958 | CB | CYS | 2834 | 40.683 | 13.755 | 13.553 | 1.00 | 27.30 |
| ATOM | 18959 | SG | CYS | 2834 | 41.516 | 15.369 | 13.665 | 1.00 | 27.45 |
| ATOM | 18960 | C | CYS | 2834 | 42.098 | 12.794 | 15.358 | 1.00 | 28.85 |
| ATOM | 18961 | O | CYS | 2834 | 41.316 | 12.957 | 16.297 | 1.00 | 28.83 |
| ATOM | 18962 | N | GLY | 2835 | 43.418 | 12.776 | 15.508 | 1.00 | 29.22 |
| ATOM | 18963 | CA | GLY | 2835 | 44.014 | 12.970 | 16.816 | 1.00 | 29.92 |
| ATOM | 18964 | C | GLY | 2835 | 43.832 | 14.382 | 17.336 | 1.00 | 30.47 |
| ATOM | 18965 | O | GLY | 2835 | 43.460 | 15.288 | 16.590 | 1.00 | 28.61 |
| ATOM | 18966 | N | HIS | 2836 | 44.100 | 14.561 | 18.624 | 1.00 | 31.84 |
| ATOM | 18967 | CA | HIS | 2836 | 43.971 | 15.860 | 19.274 | 1.00 | 35.35 |
| ATOM | 18968 | CB | HIS | 2836 | 42.553 | 16.033 | 19.816 | 1.00 | 36.73 |
| ATOM | 18969 | CG | HIS | 2836 | 42.276 | 17.396 | 20.366 | 1.00 | 38.60 |
| ATOM | 18970 | CD2 | HIS | 2836 | 42.821 | 18.067 | 21.409 | 1.00 | 39.81 |
| ATOM | 18971 | ND1 | HIS | 2836 | 41.313 | 18.226 | 19.837 | 1.00 | 39.91 |
| ATOM | 18972 | CE1 | HIS | 2836 | 41.274 | 19.350 | 20.531 | 1.00 | 41.13 |
| ATOM | 18973 | NE2 | HIS | 2836 | 42.180 | 19.278 | 21.490 | 1.00 | 41.39 |
| ATOM | 18974 | C | HIS | 2836 | 44.973 | 15.941 | 20.420 | 1.00 | 36.62 |
| ATOM | 18975 | O | HIS | 2836 | 44.800 | 15.291 | 21.452 | 1.00 | 34.99 |
| ATOM | 18976 | N | LEU | 2837 | 46.018 | 16.742 | 20.231 | 1.00 | 38.21 |
| ATOM | 18977 | CA | LEU | 2837 | 47.059 | 16.905 | 21.239 | 1.00 | 41.31 |
| ATOM | 18978 | CB | LEU | 2837 | 48.365 | 16.279 | 20.745 | 1.00 | 40.92 |
| ATOM | 18979 | CG | LEU | 2837 | 48.343 | 14.765 | 20.510 | 1.00 | 40.97 |
| ATOM | 18980 | CD1 | LEU | 2837 | 49.609 | 14.330 | 19.794 | 1.00 | 41.01 |
| ATOM | 18981 | CD2 | LEU | 2837 | 48.201 | 14.051 | 21.840 | 1.00 | 41.35 |
| ATOM | 18982 | C | LEU | 2837 | 47.285 | 18.377 | 21.557 | 1.00 | 43.49 |
| ATOM | 18983 | O | LEU | 2837 | 46.981 | 19.249 | 20.745 | 1.00 | 43.31 |
| ATOM | 18984 | N | GLY | 2838 | 47.820 | 18.647 | 22.743 | 1.00 | 46.60 |
| ATOM | 18985 | CA | GLY | 2838 | 48.077 | 20.018 | 23.141 | 1.00 | 50.65 |
| ATOM | 18986 | C | GLY | 2838 | 47.359 | 20.378 | 24.425 | 1.00 | 53.60 |
| ATOM | 18987 | O | GLY | 2838 | 47.800 | 20.013 | 25.514 | 1.00 | 54.57 |
| ATOM | 18988 | N | LEU | 2839 | 46.246 | 21.092 | 24.297 | 1.00 | 56.02 |
| ATOM | 18989 | CA | LEU | 2839 | 45.466 | 21.503 | 25.457 | 1.00 | 58.56 |
| ATOM | 18990 | CB | LEU | 2839 | 45.363 | 23.032 | 25.490 | 1.00 | 58.83 |
| ATOM | 18991 | CG | LEU | 2839 | 45.049 | 23.710 | 26.827 | 1.00 | 59.71 |
| ATOM | 18992 | CD1 | LEU | 2839 | 45.256 | 25.209 | 26.685 | 1.00 | 60.17 |
| ATOM | 18993 | CD2 | LEU | 2839 | 43.631 | 23.394 | 27.263 | 1.00 | 59.46 |
| ATOM | 18994 | C | LEU | 2839 | 44.074 | 20.875 | 25.377 | 1.00 | 60.00 |
| ATOM | 18995 | O | LEU | 2839 | 43.142 | 21.473 | 24.843 | 1.00 | 60.93 |
| ATOM | 18996 | N | THR | 2840 | 43.944 | 19.663 | 25.910 | 1.00 | 61.24 |
| ATOM | 18997 | CA | THR | 2840 | 42.672 | 18.946 | 25.892 | 1.00 | 62.45 |
| ATOM | 18998 | CB | THR | 2840 | 42.879 | 17.438 | 26.157 | 1.00 | 62.30 |
| ATOM | 18999 | OG1 | THR | 2840 | 43.477 | 17.254 | 27.446 | 1.00 | 62.47 |
| ATOM | 19000 | CG2 | THR | 2840 | 43.782 | 16.831 | 25.094 | 1.00 | 62.36 |
| ATOM | 19001 | C | THR | 2840 | 41.688 | 19.493 | 26.925 | 1.00 | 63.01 |
| ATOM | 19002 | O | THR | 2840 | 41.915 | 19.383 | 28.131 | 1.00 | 63.56 |
| ATOM | 19003 | N | PRO | 2841 | 40.577 | 20.090 | 26.460 | 1.00 | 63.41 |
| ATOM | 19004 | CD | PRO | 2841 | 40.230 | 20.321 | 25.046 | 1.00 | 63.65 |
| ATOM | 19005 | CA | PRO | 2841 | 39.553 | 20.658 | 27.343 | 1.00 | 63.37 |
| ATOM | 19006 | CB | PRO | 2841 | 38.678 | 21.457 | 26.380 | 1.00 | 63.72 |
| ATOM | 19007 | CG | PRO | 2841 | 38.754 | 20.648 | 25.127 | 1.00 | 64.24 |
| ATOM | 19008 | C | PRO | 2841 | 38.756 | 19.614 | 28.127 | 1.00 | 62.86 |
| ATOM | 19009 | O | PRO | 2841 | 38.222 | 19.911 | 29.196 | 1.00 | 62.53 |
| ATOM | 19010 | N | GLN | 2842 | 38.676 | 18.397 | 27.594 | 1.00 | 62.50 |
| ATOM | 19011 | CA | GLN | 2842 | 37.945 | 17.320 | 28.258 | 1.00 | 62.12 |
| ATOM | 19012 | CB | GLN | 2842 | 37.873 | 16.081 | 27.359 | 1.00 | 62.10 |
| ATOM | 19013 | CG | GLN | 2842 | 36.798 | 16.147 | 26.282 | 1.00 | 61.81 |
| ATOM | 19014 | CD | GLN | 2842 | 36.712 | 14.871 | 25.465 | 1.00 | 61.57 |
| ATOM | 19015 | OE1 | GLN | 2842 | 36.650 | 13.772 | 26.016 | 1.00 | 61.29 |
| ATOM | 19016 | NE2 | GLN | 2842 | 36.698 | 15.012 | 24.145 | 1.00 | 61.54 |
| ATOM | 19017 | C | GLN | 2842 | 38.583 | 16.942 | 29.591 | 1.00 | 61.81 |
| ATOM | 19018 | O | GLN | 2842 | 37.955 | 16.293 | 30.429 | 1.00 | 61.09 |
| ATOM | 19019 | N | SER | 2843 | 39.833 | 17.352 | 29.781 | 1.00 | 61.46 |
| ATOM | 19020 | CA | SER | 2843 | 40.557 | 17.060 | 31.012 | 1.00 | 61.52 |
| ATOM | 19021 | CB | SER | 2843 | 41.890 | 16.381 | 30.689 | 1.00 | 61.26 |
| ATOM | 19022 | OG | SER | 2843 | 41.690 | 15.191 | 29.947 | 1.00 | 61.14 |
| ATOM | 19023 | C | SER | 2843 | 40.810 | 18.342 | 31.798 | 1.00 | 61.57 |
| ATOM | 19024 | O | SER | 2843 | 41.905 | 18.555 | 32.320 | 1.00 | 62.21 |
| ATOM | 19025 | N | VAL | 2844 | 39.790 | 19.191 | 31.879 | 1.00 | 61.40 |
| ATOM | 19026 | CA | VAL | 2844 | 39.894 | 20.460 | 32.593 | 1.00 | 61.08 |
| ATOM | 19027 | CB | VAL | 2844 | 38.645 | 21.343 | 32.336 | 1.00 | 60.81 |
| ATOM | 19028 | CG1 | VAL | 2844 | 37.385 | 20.609 | 32.764 | 1.00 | 60.34 |
| ATOM | 19029 | CG2 | VAL | 2844 | 38.775 | 22.662 | 33.080 | 1.00 | 60.79 |
| ATOM | 19030 | C | VAL | 2844 | 40.066 | 20.259 | 34.101 | 1.00 | 61.01 |
| ATOM | 19031 | O | VAL | 2844 | 40.803 | 21.001 | 34.754 | 1.00 | 60.58 |
| ATOM | 19032 | N | ASN | 2845 | 39.389 | 19.251 | 34.644 | 1.00 | 61.15 |
| ATOM | 19033 | CA | ASN | 2845 | 39.460 | 18.947 | 36.071 | 1.00 | 61.73 |
| ATOM | 19034 | CB | ASN | 2845 | 38.364 | 17.946 | 36.449 | 1.00 | 60.56 |
| ATOM | 19035 | CG | ASN | 2845 | 36.970 | 18.499 | 36.230 | 1.00 | 59.61 |
| ATOM | 19036 | OD1 | ASN | 2845 | 36.548 | 19.436 | 36.907 | 1.00 | 58.50 |
| ATOM | 19037 | ND2 | ASN | 2845 | 36.249 | 17.924 | 35.275 | 1.00 | 59.70 |
| ATOM | 19038 | C | ASN | 2845 | 40.821 | 18.388 | 36.475 | 1.00 | 62.60 |
| ATOM | 19039 | O | ASN | 2845 | 41.296 | 18.629 | 37.586 | 1.00 | 62.03 |
| ATOM | 19040 | N | ILE | 2846 | 41.441 | 17.636 | 35.570 | 1.00 | 64.05 |
| ATOM | 19041 | CA | ILE | 2846 | 42.750 | 17.046 | 35.831 | 1.00 | 65.55 |
| ATOM | 19042 | CB | ILE | 2846 | 43.208 | 16.151 | 34.651 | 1.00 | 65.57 |
| ATOM | 19043 | CG2 | ILE | 2846 | 44.609 | 15.612 | 34.915 | 1.00 | 65.70 |
| ATOM | 19044 | CG1 | ILE | 2846 | 42.223 | 14.995 | 34.453 | 1.00 | 65.59 |
| ATOM | 19045 | CD1 | ILE | 2846 | 42.118 | 14.057 | 35.643 | 1.00 | 65.41 |
| ATOM | 19046 | C | ILE | 2846 | 43.795 | 18.137 | 36.051 | 1.00 | 66.57 |
| ATOM | 19047 | O | ILE | 2846 | 44.490 | 18.150 | 37.066 | 1.00 | 66.67 |
| ATOM | 19048 | N | PHE | 2847 | 43.896 | 19.053 | 35.092 | 1.00 | 67.71 |
| ATOM | 19049 | CA | PHE | 2847 | 44.855 | 20.149 | 35.172 | 1.00 | 68.89 |
| ATOM | 19050 | CB | PHE | 2847 | 45.158 | 20.684 | 33.771 | 1.00 | 69.22 |
| ATOM | 19051 | CG | PHE | 2847 | 45.618 | 19.630 | 32.807 | 1.00 | 69.88 |
| ATOM | 19052 | CD1 | PHE | 2847 | 46.776 | 18.899 | 33.055 | 1.00 | 70.09 |
| ATOM | 19053 | CD2 | PHE | 2847 | 44.892 | 19.365 | 31.649 | 1.00 | 70.08 |
| ATOM | 19054 | CE1 | PHE | 2847 | 47.206 | 17.918 | 32.164 | 1.00 | 70.40 |
| ATOM | 19055 | CE2 | PHE | 2847 | 45.311 | 18.387 | 30.751 | 1.00 | 70.36 |
| ATOM | 19056 | CZ | PHE | 2847 | 46.471 | 17.661 | 31.009 | 1.00 | 70.74 |
| ATOM | 19057 | C | PHE | 2847 | 44.333 | 21.284 | 36.050 | 1.00 | 69.49 |
| ATOM | 19058 | O | PHE | 2847 | 45.040 | 22.262 | 36.299 | 1.00 | 69.68 |
| ATOM | 19059 | N | GLY | 2848 | 43.094 | 21.149 | 36.513 | 1.00 | 69.83 |
| ATOM | 19060 | CA | GLY | 2848 | 42.502 | 22.174 | 37.353 | 1.00 | 70.24 |
| ATOM | 19061 | C | GLY | 2848 | 42.298 | 23.478 | 36.605 | 1.00 | 70.50 |
| ATOM | 19062 | O | GLY | 2848 | 42.086 | 24.528 | 37.212 | 1.00 | 70.31 |
| ATOM | 19063 | N | GLY | 2849 | 42.363 | 23.407 | 35.280 | 1.00 | 71.04 |
| ATOM | 19064 | CA | GLY | 2849 | 42.185 | 24.591 | 34.459 | 1.00 | 71.60 |
| ATOM | 19065 | C | GLY | 2849 | 42.700 | 24.367 | 33.051 | 1.00 | 72.16 |
| ATOM | 19066 | O | GLY | 2849 | 42.776 | 23.228 | 32.589 | 1.00 | 72.14 |
| ATOM | 19067 | N | TYR | 2850 | 43.056 | 25.450 | 32.366 | 1.00 | 72.95 |
| ATOM | 19068 | CA | TYR | 2850 | 43.570 | 25.353 | 31.005 | 1.00 | 73.65 |
| ATOM | 19069 | CB | TYR | 2850 | 42.679 | 26.142 | 30.038 | 1.00 | 73.69 |
| ATOM | 19070 | CG | TYR | 2850 | 41.257 | 25.633 | 29.969 | 1.00 | 74.01 |
| ATOM | 19071 | CD1 | TYR | 2850 | 40.353 | 25.887 | 31.001 | 1.00 | 74.35 |
| ATOM | 19072 | CE1 | TYR | 2850 | 39.050 | 25.395 | 30.956 | 1.00 | 74.49 |
| ATOM | 19073 | CD2 | TYR | 2850 | 40.821 | 24.871 | 28.883 | 1.00 | 74.02 |
| ATOM | 19074 | CE2 | TYR | 2850 | 39.520 | 24.373 | 28.828 | 1.00 | 74.41 |
| ATOM | 19075 | CZ | TYR | 2850 | 38.641 | 24.639 | 29.868 | 1.00 | 74.86 |
| ATOM | 19076 | OH | TYR | 2850 | 37.355 | 24.146 | 29.826 | 1.00 | 75.22 |
| ATOM | 19077 | C | TYR | 2850 | 45.007 | 25.853 | 30.907 | 1.00 | 74.10 |
| ATOM | 19078 | O | TYR | 2850 | 45.256 | 27.059 | 30.847 | 1.00 | 74.16 |
| ATOM | 19079 | N | LYS | 2851 | 45.947 | 24.913 | 30.892 | 1.00 | 74.74 |
| ATOM | 19080 | CA | LYS | 2851 | 47.369 | 25.229 | 30.801 | 1.00 | 75.33 |
| ATOM | 19081 | CB | LYS | 2851 | 48.166 | 24.299 | 31.719 | 1.00 | 75.40 |
| ATOM | 19082 | CG | LYS | 2851 | 47.704 | 24.325 | 33.168 | 1.00 | 75.61 |
| ATOM | 19083 | CD | LYS | 2851 | 48.399 | 23.258 | 33.998 | 1.00 | 75.38 |
| ATOM | 19084 | CE | LYS | 2851 | 47.875 | 23.249 | 35.426 | 1.00 | 75.26 |
| ATOM | 19085 | NZ | LYS | 2851 | 48.476 | 22.152 | 36.233 | 1.00 | 74.92 |
| ATOM | 19086 | C | LYS | 2851 | 47.845 | 25.070 | 29.358 | 1.00 | 75.66 |
| ATOM | 19087 | O | LYS | 2851 | 47.572 | 24.057 | 28.714 | 1.00 | 76.01 |
| ATOM | 19088 | N | VAL | 2852 | 48.557 | 26.073 | 28.857 | 1.00 | 75.85 |
| ATOM | 19089 | CA | VAL | 2852 | 49.062 | 26.043 | 27.489 | 1.00 | 76.08 |
| ATOM | 19090 | CB | VAL | 2852 | 49.525 | 27.448 | 27.036 | 1.00 | 75.99 |
| ATOM | 19091 | CG1 | VAL | 2852 | 49.922 | 27.421 | 25.568 | 1.00 | 75.65 |
| ATOM | 19092 | CG2 | VAL | 2852 | 48.417 | 28.463 | 27.273 | 1.00 | 75.82 |
| ATOM | 19093 | C | VAL | 2852 | 50.234 | 25.074 | 27.346 | 1.00 | 76.39 |
| ATOM | 19094 | O | VAL | 2852 | 51.395 | 25.484 | 27.394 | 1.00 | 76.33 |
| ATOM | 19095 | N | GLN | 2853 | 49.930 | 23.789 | 27.173 | 1.00 | 76.70 |
| ATOM | 19096 | CA | GLN | 2853 | 50.971 | 22.777 | 27.016 | 1.00 | 77.12 |
| ATOM | 19097 | CB | GLN | 2853 | 50.360 | 21.375 | 26.913 | 1.00 | 77.23 |
| ATOM | 19098 | CG | GLN | 2853 | 50.094 | 20.680 | 28.247 | 1.00 | 76.94 |
| ATOM | 19099 | CD | GLN | 2853 | 48.944 | 21.289 | 29.021 | 1.00 | 76.79 |
| ATOM | 19100 | OE1 | GLN | 2853 | 47.825 | 21.379 | 28.519 | 1.00 | 76.67 |
| ATOM | 19101 | NE2 | GLN | 2853 | 49.210 | 21.699 | 30.256 | 1.00 | 77.11 |
| ATOM | 19102 | C | GLN | 2853 | 51.813 | 23.054 | 25.774 | 1.00 | 77.33 |
| ATOM | 19103 | O | GLN | 2853 | 51.369 | 23.740 | 24.853 | 1.00 | 77.15 |
| ATOM | 19104 | N | GLY | 2854 | 53.027 | 22.514 | 25.755 | 1.00 | 77.75 |
| ATOM | 19105 | CA | GLY | 2854 | 53.911 | 22.717 | 24.622 | 1.00 | 78.29 |
| ATOM | 19106 | C | GLY | 2854 | 55.254 | 23.293 | 25.028 | 1.00 | 78.76 |
| ATOM | 19107 | O | GLY | 2854 | 56.190 | 23.331 | 24.228 | 1.00 | 78.78 |
| ATOM | 19108 | N | ARG | 2855 | 55.348 | 23.744 | 26.276 | 1.00 | 79.02 |
| ATOM | 19109 | CA | ARG | 2855 | 56.584 | 24.320 | 26.800 | 1.00 | 79.07 |
| ATOM | 19110 | CB | ARG | 2855 | 56.271 | 25.282 | 27.954 | 1.00 | 79.32 |
| ATOM | 19111 | CG | ARG | 2855 | 55.397 | 26.471 | 27.572 | 1.00 | 79.76 |
| ATOM | 19112 | CD | ARG | 2855 | 56.115 | 27.420 | 26.620 | 1.00 | 80.09 |
| ATOM | 19113 | NE | ARG | 2855 | 55.254 | 28.516 | 26.174 | 1.00 | 80.24 |
| ATOM | 19114 | CZ | ARG | 2855 | 54.727 | 29.437 | 26.976 | 1.00 | 79.98 |
| ATOM | 19115 | NH1 | ARG | 2855 | 54.968 | 29.406 | 28.280 | 1.00 | 79.86 |
| ATOM | 19116 | NH2 | ARG | 2855 | 53.956 | 30.393 | 26.473 | 1.00 | 79.79 |
| ATOM | 19117 | C | ARG | 2855 | 57.524 | 23.222 | 27.293 | 1.00 | 78.89 |
| ATOM | 19118 | O | ARG | 2855 | 57.079 | 22.176 | 27.767 | 1.00 | 78.88 |
| ATOM | 19119 | N | GLY | 2856 | 58.826 | 23.468 | 27.179 | 1.00 | 78.53 |
| ATOM | 19120 | CA | GLY | 2856 | 59.805 | 22.492 | 27.619 | 1.00 | 78.02 |
| ATOM | 19121 | C | GLY | 2856 | 60.118 | 21.456 | 26.557 | 1.00 | 77.79 |
| ATOM | 19122 | O | GLY | 2856 | 59.439 | 21.382 | 25.532 | 1.00 | 77.80 |
| ATOM | 19123 | N | ASP | 2857 | 61.150 | 20.655 | 26.802 | 1.00 | 77.36 |
| ATOM | 19124 | CA | ASP | 2857 | 61.555 | 19.616 | 25.861 | 1.00 | 76.70 |
| ATOM | 19125 | CB | ASP | 2857 | 63.077 | 19.446 | 25.875 | 1.00 | 76.96 |
| ATOM | 19126 | CG | ASP | 2857 | 63.801 | 20.634 | 25.273 | 1.00 | 77.36 |
| ATOM | 19127 | OD1 | ASP | 2857 | 63.527 | 20.962 | 24.099 | 1.00 | 77.51 |
| ATOM | 19128 | OD2 | ASP | 2857 | 64.644 | 21.235 | 25.971 | 1.00 | 77.38 |
| ATOM | 19129 | C | ASP | 2857 | 60.892 | 18.282 | 26.177 | 1.00 | 75.97 |
| ATOM | 19130 | O | ASP | 2857 | 60.391 | 17.606 | 25.280 | 1.00 | 76.56 |
| ATOM | 19131 | N | GLU | 2858 | 60.896 | 17.901 | 27.450 | 1.00 | 74.75 |
| ATOM | 19132 | CA | GLU | 2858 | 60.288 | 16.644 | 27.863 | 1.00 | 73.64 |
| ATOM | 19133 | CB | GLU | 2858 | 60.368 | 16.484 | 29.383 | 1.00 | 74.08 |
| ATOM | 19134 | CG | GLU | 2858 | 59.765 | 15.184 | 29.899 | 1.00 | 74.86 |
| ATOM | 19135 | CD | GLU | 2858 | 59.856 | 15.050 | 31.408 | 1.00 | 75.46 |
| ATOM | 19136 | OE1 | GLU | 2858 | 60.986 | 15.074 | 31.941 | 1.00 | 75.97 |
| ATOM | 19137 | OE2 | GLU | 2858 | 58.798 | 14.918 | 32.061 | 1.00 | 75.55 |
| ATOM | 19138 | C | GLU | 2858 | 58.831 | 16.596 | 27.416 | 1.00 | 72.68 |
| ATOM | 19139 | O | GLU | 2858 | 58.319 | 15.537 | 27.056 | 1.00 | 72.94 |
| ATOM | 19140 | N | ALA | 2859 | 58.172 | 17.751 | 27.441 | 1.00 | 71.17 |
| ATOM | 19141 | CA | ALA | 2859 | 56.774 | 17.848 | 27.036 | 1.00 | 69.66 |
| ATOM | 19142 | CB | ALA | 2859 | 56.091 | 18.987 | 27.786 | 1.00 | 69.37 |
| ATOM | 19143 | C | ALA | 2859 | 56.682 | 18.083 | 25.534 | 1.00 | 68.42 |
| ATOM | 19144 | O | ALA | 2859 | 55.790 | 17.560 | 24.864 | 1.00 | 68.24 |
| ATOM | 19145 | N | GLY | 2860 | 57.613 | 18.876 | 25.014 | 1.00 | 67.04 |
| ATOM | 19146 | CA | GLY | 2860 | 57.628 | 19.171 | 23.595 | 1.00 | 65.45 |
| ATOM | 19147 | C | GLY | 2860 | 57.836 | 17.929 | 22.753 | 1.00 | 64.41 |
| ATOM | 19148 | O | GLY | 2860 | 57.153 | 17.734 | 21.748 | 1.00 | 64.15 |
| ATOM | 19149 | N | ASP | 2861 | 58.782 | 17.087 | 23.160 | 1.00 | 63.48 |
| ATOM | 19150 | CA | ASP | 2861 | 59.070 | 15.856 | 22.433 | 1.00 | 62.53 |
| ATOM | 19151 | CB | ASP | 2861 | 60.439 | 15.302 | 22.831 | 1.00 | 62.67 |
| ATOM | 19152 | CG | ASP | 2861 | 61.562 | 16.288 | 22.579 | 1.00 | 63.21 |
| ATOM | 19153 | OD1 | ASP | 2861 | 61.615 | 16.865 | 21.471 | 1.00 | 62.95 |
| ATOM | 19154 | OD2 | ASP | 2861 | 62.397 | 16.478 | 23.487 | 1.00 | 63.46 |
| ATOM | 19155 | C | ASP | 2861 | 57.999 | 14.811 | 22.716 | 1.00 | 61.69 |
| ATOM | 19156 | O | ASP | 2861 | 57.830 | 13.860 | 21.953 | 1.00 | 61.58 |
| ATOM | 19157 | N | GLN | 2862 | 57.285 | 14.992 | 23.822 | 1.00 | 60.47 |
| ATOM | 19158 | CA | GLN | 2862 | 56.221 | 14.073 | 24.207 | 1.00 | 59.44 |
| ATOM | 19159 | CB | GLN | 2862 | 55.729 | 14.389 | 25.620 | 1.00 | 59.54 |
| ATOM | 19160 | CG | GLN | 2862 | 54.527 | 13.569 | 26.050 | 1.00 | 59.67 |
| ATOM | 19161 | CD | GLN | 2862 | 54.800 | 12.080 | 26.016 | 1.00 | 59.97 |
| ATOM | 19162 | OE1 | GLN | 2862 | 55.653 | 11.578 | 26.748 | 1.00 | 60.86 |
| ATOM | 19163 | NE2 | GLN | 2862 | 54.078 | 11.366 | 25.160 | 1.00 | 59.63 |
| ATOM | 19164 | C | GLN | 2862 | 55.061 | 14.194 | 23.227 | 1.00 | 58.25 |
| ATOM | 19165 | O | GLN | 2862 | 54.494 | 13.193 | 22.797 | 1.00 | 58.14 |
| ATOM | 19166 | N | LEU | 2863 | 54.712 | 15.429 | 22.884 | 1.00 | 57.06 |
| ATOM | 19167 | CA | LEU | 2863 | 53.625 | 15.679 | 21.951 | 1.00 | 56.74 |
| ATOM | 19168 | CB | LEU | 2863 | 53.350 | 17.181 | 21.844 | 1.00 | 57.27 |
| ATOM | 19169 | CG | LEU | 2863 | 52.854 | 17.884 | 23.111 | 1.00 | 57.78 |
| ATOM | 19170 | CD1 | LEU | 2863 | 52.796 | 19.385 | 22.873 | 1.00 | 58.08 |
| ATOM | 19171 | CD2 | LEU | 2863 | 51.485 | 17.347 | 23.497 | 1.00 | 57.79 |
| ATOM | 19172 | C | LEU | 2863 | 53.988 | 15.128 | 20.580 | 1.00 | 56.01 |
| ATOM | 19173 | O | LEU | 2863 | 53.163 | 14.504 | 19.911 | 1.00 | 56.51 |
| ATOM | 19174 | N | LEU | 2864 | 55.230 | 15.358 | 20.168 | 1.00 | 54.58 |
| ATOM | 19175 | CA | LEU | 2864 | 55.700 | 14.882 | 18.873 | 1.00 | 53.21 |
| ATOM | 19176 | CB | LEU | 2864 | 57.106 | 15.427 | 18.596 | 1.00 | 53.69 |
| ATOM | 19177 | CG | LEU | 2864 | 57.598 | 15.447 | 17.145 | 1.00 | 54.10 |
| ATOM | 19178 | CD1 | LEU | 2864 | 58.822 | 16.342 | 17.047 | 1.00 | 54.57 |
| ATOM | 19179 | CD2 | LEU | 2864 | 57.911 | 14.043 | 16.669 | 1.00 | 53.90 |
| ATOM | 19180 | C | LEU | 2864 | 55.707 | 13.355 | 18.879 | 1.00 | 51.65 |
| ATOM | 19181 | O | LEU | 2864 | 55.486 | 12.717 | 17.847 | 1.00 | 50.82 |
| ATOM | 19182 | N | SER | 2865 | 55.954 | 12.781 | 20.053 | 1.00 | 49.04 |
| ATOM | 19183 | CA | SER | 2865 | 55.975 | 11.332 | 20.209 | 1.00 | 47.06 |
| ATOM | 19184 | CB | SER | 2865 | 56.542 | 10.954 | 21.581 | 1.00 | 46.62 |
| ATOM | 19185 | OG | SER | 2865 | 56.593 | 9.548 | 21.749 | 1.00 | 44.01 |
| ATOM | 19186 | C | SER | 2865 | 54.561 | 10.774 | 20.062 | 1.00 | 45.80 |
| ATOM | 19187 | O | SER | 2865 | 54.346 | 9.778 | 19.367 | 1.00 | 44.53 |
| ATOM | 19188 | N | ASP | 2866 | 53.600 | 11.417 | 20.720 | 1.00 | 44.91 |
| ATOM | 19189 | CA | ASP | 2866 | 52.210 | 10.976 | 20.645 | 1.00 | 44.58 |
| ATOM | 19190 | CB | ASP | 2866 | 51.329 | 11.752 | 21.631 | 1.00 | 45.13 |
| ATOM | 19191 | CG | ASP | 2866 | 51.719 | 11.514 | 23.080 | 1.00 | 45.03 |
| ATOM | 19192 | OD1 | ASP | 2866 | 51.989 | 10.352 | 23.452 | 1.00 | 44.80 |
| ATOM | 19193 | OD2 | ASP | 2866 | 51.740 | 12.489 | 23.855 | 1.00 | 46.12 |
| ATOM | 19194 | C | ASP | 2866 | 51.687 | 11.180 | 19.229 | 1.00 | 44.24 |
| ATOM | 19195 | O | ASP | 2866 | 50.975 | 10.329 | 18.694 | 1.00 | 45.01 |
| ATOM | 19196 | N | ALA | 2867 | 52.043 | 12.310 | 18.626 | 1.00 | 42.65 |
| ATOM | 19197 | CA | ALA | 2867 | 51.612 | 12.610 | 17.267 | 1.00 | 41.30 |
| ATOM | 19198 | CB | ALA | 2867 | 52.178 | 13.952 | 16.820 | 1.00 | 40.89 |
| ATOM | 19199 | C | ALA | 2867 | 52.077 | 11.498 | 16.328 | 1.00 | 40.67 |
| ATOM | 19200 | O | ALA | 2867 | 51.325 | 11.051 | 15.461 | 1.00 | 38.64 |
| ATOM | 19201 | N | LEU | 2868 | 53.318 | 11.052 | 16.506 | 1.00 | 39.50 |
| ATOM | 19202 | CA | LEU | 2868 | 53.862 | 9.982 | 15.674 | 1.00 | 39.13 |
| ATOM | 19203 | CB | LEU | 2868 | 55.375 | 9.852 | 15.881 | 1.00 | 39.80 |
| ATOM | 19204 | CG | LEU | 2868 | 56.263 | 10.908 | 15.223 | 1.00 | 39.60 |
| ATOM | 19205 | CD1 | LEU | 2868 | 57.701 | 10.724 | 15.682 | 1.00 | 40.79 |
| ATOM | 19206 | CD2 | LEU | 2868 | 56.167 | 10.790 | 13.709 | 1.00 | 40.12 |
| ATOM | 19207 | C | LEU | 2868 | 53.189 | 8.655 | 16.003 | 1.00 | 38.24 |
| ATOM | 19208 | O | LEU | 2868 | 52.981 | 7.817 | 15.124 | 1.00 | 37.72 |
| ATOM | 19209 | N | ALA | 2869 | 52.853 | 8.470 | 17.275 | 1.00 | 36.58 |
| ATOM | 19210 | CA | ALA | 2869 | 52.197 | 7.246 | 17.723 | 1.00 | 36.98 |
| ATOM | 19211 | CB | ALA | 2869 | 52.156 | 7.206 | 19.245 | 1.00 | 36.32 |
| ATOM | 19212 | C | ALA | 2869 | 50.782 | 7.165 | 17.157 | 1.00 | 36.13 |
| ATOM | 19213 | O | ALA | 2869 | 50.346 | 6.109 | 16.699 | 1.00 | 37.03 |
| ATOM | 19214 | N | LEU | 2870 | 50.070 | 8.287 | 17.195 | 1.00 | 35.62 |
| ATOM | 19215 | CA | LEU | 2870 | 48.705 | 8.349 | 16.684 | 1.00 | 35.07 |
| ATOM | 19216 | CB | LEU | 2870 | 48.091 | 9.719 | 16.990 | 1.00 | 35.82 |
| ATOM | 19217 | CG | LEU | 2870 | 47.905 | 10.070 | 18.471 | 1.00 | 36.77 |
| ATOM | 19218 | CD1 | LEU | 2870 | 47.517 | 11.530 | 18.606 | 1.00 | 37.65 |
| ATOM | 19219 | CD2 | LEU | 2870 | 46.842 | 9.171 | 19.087 | 1.00 | 37.97 |
| ATOM | 19220 | C | LEU | 2870 | 48.703 | 8.099 | 15.180 | 1.00 | 33.74 |
| ATOM | 19221 | O | LEU | 2870 | 47.826 | 7.413 | 14.655 | 1.00 | 32.74 |
| ATOM | 19222 | N | GLU | 2871 | 49.692 | 8.656 | 14.489 | 1.00 | 33.03 |
| ATOM | 19223 | CA | GLU | 2871 | 49.807 | 8.480 | 13.047 | 1.00 | 31.65 |
| ATOM | 19224 | CB | GLU | 2871 | 50.952 | 9.338 | 12.501 | 1.00 | 32.99 |
| ATOM | 19225 | CG | GLU | 2871 | 51.193 | 9.165 | 11.011 | 1.00 | 33.67 |
| ATOM | 19226 | CD | GLU | 2871 | 52.375 | 9.974 | 10.518 | 1.00 | 34.78 |
| ATOM | 19227 | OE1 | GLU | 2871 | 53.477 | 9.806 | 11.085 | 1.00 | 33.73 |
| ATOM | 19228 | OE2 | GLU | 2871 | 52.205 | 10.766 | 9.567 | 1.00 | 35.40 |
| ATOM | 19229 | C | GLU | 2871 | 50.061 | 7.015 | 12.715 | 1.00 | 31.63 |
| ATOM | 19230 | O | GLU | 2871 | 49.438 | 6.460 | 11.811 | 1.00 | 31.20 |
| ATOM | 19231 | N | ALA | 2872 | 50.975 | 6.391 | 13.451 | 1.00 | 30.57 |
| ATOM | 19232 | CA | ALA | 2872 | 51.306 | 4.988 | 13.221 | 1.00 | 30.98 |
| ATOM | 19233 | CB | ALA | 2872 | 52.506 | 4.588 | 14.076 | 1.00 | 30.03 |
| ATOM | 19234 | C | ALA | 2872 | 50.110 | 4.095 | 13.544 | 1.00 | 29.80 |
| ATOM | 19235 | O | ALA | 2872 | 49.949 | 3.020 | 12.965 | 1.00 | 28.80 |
| ATOM | 19236 | N | ALA | 2873 | 49.274 | 4.561 | 14.468 | 1.00 | 29.24 |
| ATOM | 19237 | CA | ALA | 2873 | 48.086 | 3.832 | 14.898 | 1.00 | 27.99 |
| ATOM | 19238 | CB | ALA | 2873 | 47.538 | 4.449 | 16.185 | 1.00 | 27.72 |
| ATOM | 19239 | C | ALA | 2873 | 47.010 | 3.825 | 13.818 | 1.00 | 28.11 |
| ATOM | 19240 | O | ALA | 2873 | 46.158 | 2.935 | 13.782 | 1.00 | 27.45 |
| ATOM | 19241 | N | GLY | 2874 | 47.044 | 4.821 | 12.939 | 1.00 | 26.89 |
| ATOM | 19242 | CA | GLY | 2874 | 46.059 | 4.883 | 11.874 | 1.00 | 27.96 |
| ATOM | 19243 | C | GLY | 2874 | 45.396 | 6.235 | 11.685 | 1.00 | 27.38 |
| ATOM | 19244 | O | GLY | 2874 | 44.639 | 6.428 | 10.736 | 1.00 | 27.45 |
| ATOM | 19245 | N | ALA | 2875 | 45.671 | 7.175 | 12.583 | 1.00 | 28.20 |
| ATOM | 19246 | CA | ALA | 2875 | 45.085 | 8.502 | 12.481 | 1.00 | 28.33 |
| ATOM | 19247 | CB | ALA | 2875 | 45.556 | 9.366 | 13.640 | 1.00 | 29.66 |
| ATOM | 19248 | C | ALA | 2875 | 45.462 | 9.154 | 11.147 | 1.00 | 30.07 |
| ATOM | 19249 | O | ALA | 2875 | 46.634 | 9.176 | 10.768 | 1.00 | 30.07 |
| ATOM | 19250 | N | GLN | 2876 | 44.466 | 9.679 | 10.436 | 1.00 | 29.85 |
| ATOM | 19251 | CA | GLN | 2876 | 44.704 | 10.324 | 9.146 | 1.00 | 31.07 |
| ATOM | 19252 | CB | GLN | 2876 | 43.634 | 9.895 | 8.141 | 1.00 | 32.52 |
| ATOM | 19253 | CG | GLN | 2876 | 43.582 | 8.393 | 7.917 | 1.00 | 33.27 |
| ATOM | 19254 | CD | GLN | 2876 | 42.514 | 7.983 | 6.922 | 1.00 | 35.64 |
| ATOM | 19255 | OE1 | GLN | 2876 | 42.635 | 8.238 | 5.720 | 1.00 | 37.92 |
| ATOM | 19256 | NE2 | GLN | 2876 | 41.459 | 7.346 | 7.417 | 1.00 | 34.07 |
| ATOM | 19257 | C | GLN | 2876 | 44.716 | 11.842 | 9.270 | 1.00 | 32.63 |
| ATOM | 19258 | O | GLN | 2876 | 44.871 | 12.556 | 8.278 | 1.00 | 32.11 |
| ATOM | 19259 | N | LEU | 2877 | 44.560 | 12.321 | 10.500 | 1.00 | 34.15 |
| ATOM | 19260 | CA | LEU | 2877 | 44.547 | 13.750 | 10.794 | 1.00 | 35.91 |
| ATOM | 19261 | CB | LEU | 2877 | 43.154 | 14.329 | 10.547 | 1.00 | 36.23 |
| ATOM | 19262 | CG | LEU | 2877 | 42.960 | 15.163 | 9.283 | 1.00 | 37.74 |
| ATOM | 19263 | CD1 | LEU | 2877 | 41.505 | 15.594 | 9.176 | 1.00 | 37.26 |
| ATOM | 19264 | CD2 | LEU | 2877 | 43.877 | 16.373 | 9.332 | 1.00 | 37.89 |
| ATOM | 19265 | C | LEU | 2877 | 44.933 | 13.990 | 12.244 | 1.00 | 36.30 |
| ATOM | 19266 | O | LEU | 2877 | 44.750 | 13.117 | 13.090 | 1.00 | 36.32 |
| ATOM | 19267 | N | LEU | 2878 | 45.465 | 15.178 | 12.522 | 1.00 | 37.48 |
| ATOM | 19268 | CA | LEU | 2878 | 45.875 | 15.555 | 13.873 | 1.00 | 38.35 |
| ATOM | 19269 | CB | LEU | 2878 | 47.379 | 15.346 | 14.057 | 1.00 | 37.59 |
| ATOM | 19270 | CG | LEU | 2878 | 47.944 | 15.762 | 15.418 | 1.00 | 38.15 |
| ATOM | 19271 | CD1 | LEU | 2878 | 47.332 | 14.906 | 16.517 | 1.00 | 37.53 |
| ATOM | 19272 | CD2 | LEU | 2878 | 49.458 | 15.611 | 15.411 | 1.00 | 38.68 |
| ATOM | 19273 | C | LEU | 2878 | 45.535 | 17.017 | 14.151 | 1.00 | 39.83 |
| ATOM | 19274 | O | LEU | 2878 | 45.609 | 17.861 | 13.257 | 1.00 | 39.08 |
| ATOM | 19275 | N | VAL | 2879 | 45.162 | 17.309 | 15.393 | 1.00 | 41.38 |
| ATOM | 19276 | CA | VAL | 2879 | 44.820 | 18.670 | 15.793 | 1.00 | 42.90 |
| ATOM | 19277 | CB | VAL | 2879 | 43.342 | 18.778 | 16.231 | 1.00 | 42.29 |
| ATOM | 19278 | CG1 | VAL | 2879 | 43.035 | 20.196 | 16.685 | 1.00 | 42.71 |
| ATOM | 19279 | CG2 | VAL | 2879 | 42.433 | 18.384 | 15.086 | 1.00 | 41.99 |
| ATOM | 19280 | C | VAL | 2879 | 45.700 | 19.133 | 16.949 | 1.00 | 44.01 |
| ATOM | 19281 | O | VAL | 2879 | 45.791 | 18.468 | 17.983 | 1.00 | 43.86 |
| ATOM | 19282 | N | LEU | 2880 | 46.352 | 20.276 | 16.760 | 1.00 | 45.45 |
| ATOM | 19283 | CA | LEU | 2880 | 47.221 | 20.852 | 17.781 | 1.00 | 47.07 |
| ATOM | 19284 | CB | LEU | 2880 | 48.564 | 21.261 | 17.170 | 1.00 | 47.30 |
| ATOM | 19285 | CG | LEU | 2880 | 49.446 | 20.136 | 16.632 | 1.00 | 47.79 |
| ATOM | 19286 | CD1 | LEU | 2880 | 50.688 | 20.726 | 15.975 | 1.00 | 48.71 |
| ATOM | 19287 | CD2 | LEU | 2880 | 49.832 | 19.201 | 17.770 | 1.00 | 47.63 |
| ATOM | 19288 | C | LEU | 2880 | 46.539 | 22.073 | 18.381 | 1.00 | 48.19 |
| ATOM | 19289 | O | LEU | 2880 | 46.213 | 23.022 | 17.667 | 1.00 | 48.54 |
| ATOM | 19290 | N | GLU | 2881 | 46.326 | 22.047 | 19.692 | 1.00 | 49.26 |
| ATOM | 19291 | CA | GLU | 2881 | 45.668 | 23.154 | 20.377 | 1.00 | 51.27 |
| ATOM | 19292 | CB | GLU | 2881 | 44.478 | 22.635 | 21.186 | 1.00 | 51.11 |
| ATOM | 19293 | CG | GLU | 2881 | 43.547 | 23.724 | 21.684 | 1.00 | 52.03 |
| ATOM | 19294 | CD | GLU | 2881 | 42.497 | 23.196 | 22.631 | 1.00 | 51.94 |
| ATOM | 19295 | OE1 | GLU | 2881 | 41.810 | 22.220 | 22.271 | 1.00 | 51.97 |
| ATOM | 19296 | OE2 | GLU | 2881 | 42.358 | 23.756 | 23.740 | 1.00 | 53.44 |
| ATOM | 19297 | C | GLU | 2881 | 46.629 | 23.894 | 21.304 | 1.00 | 52.45 |
| ATOM | 19298 | O | GLU | 2881 | 47.177 | 23.308 | 22.239 | 1.00 | 51.90 |
| ATOM | 19299 | N | CYS | 2882 | 46.818 | 25.185 | 21.038 | 1.00 | 53.90 |
| ATOM | 19300 | CA | CYS | 2882 | 47.705 | 26.033 | 21.832 | 1.00 | 55.11 |
| ATOM | 19301 | CB | CYS | 2882 | 47.038 | 26.398 | 23.159 | 1.00 | 55.59 |
| ATOM | 19302 | SG | CYS | 2882 | 45.596 | 27.473 | 22.965 | 1.00 | 57.79 |
| ATOM | 19303 | C | CYS | 2882 | 49.054 | 25.381 | 22.095 | 1.00 | 55.25 |
| ATOM | 19304 | O | CYS | 2882 | 49.260 | 24.729 | 23.119 | 1.00 | 55.61 |
| ATOM | 19305 | N | VAL | 2883 | 49.974 | 25.570 | 21.158 | 1.00 | 55.88 |
| ATOM | 19306 | CA | VAL | 2883 | 51.312 | 25.006 | 21.264 | 1.00 | 56.54 |
| ATOM | 19307 | CB | VAL | 2883 | 51.364 | 23.606 | 20.601 | 1.00 | 56.77 |
| ATOM | 19308 | CG1 | VAL | 2883 | 50.923 | 23.700 | 19.150 | 1.00 | 57.14 |
| ATOM | 19309 | CG2 | VAL | 2883 | 52.764 | 23.029 | 20.694 | 1.00 | 56.92 |
| ATOM | 19310 | C | VAL | 2883 | 52.291 | 25.949 | 20.568 | 1.00 | 56.70 |
| ATOM | 19311 | O | VAL | 2883 | 51.988 | 26.489 | 19.502 | 1.00 | 56.49 |
| ATOM | 19312 | N | PRO | 2884 | 53.476 | 26.168 | 21.167 | 1.00 | 56.87 |
| ATOM | 19313 | CD | PRO | 2884 | 54.038 | 25.483 | 22.342 | 1.00 | 56.82 |
| ATOM | 19314 | CA | PRO | 2884 | 54.473 | 27.060 | 20.565 | 1.00 | 56.93 |
| ATOM | 19315 | CB | PRO | 2884 | 55.699 | 26.863 | 21.459 | 1.00 | 56.72 |
| ATOM | 19316 | CG | PRO | 2884 | 55.503 | 25.482 | 22.019 | 1.00 | 57.13 |
| ATOM | 19317 | C | PRO | 2884 | 54.747 | 26.718 | 19.105 | 1.00 | 57.27 |
| ATOM | 19318 | O | PRO | 2884 | 54.999 | 25.562 | 18.766 | 1.00 | 57.23 |
| ATOM | 19319 | N | VAL | 2885 | 54.689 | 27.735 | 18.250 | 1.00 | 56.98 |
| ATOM | 19320 | CA | VAL | 2885 | 54.908 | 27.564 | 16.818 | 1.00 | 57.68 |
| ATOM | 19321 | CB | VAL | 2885 | 55.176 | 28.918 | 16.129 | 1.00 | 57.41 |
| ATOM | 19322 | CG1 | VAL | 2885 | 55.141 | 28.747 | 14.620 | 1.00 | 57.11 |
| ATOM | 19323 | CG2 | VAL | 2885 | 54.150 | 29.942 | 16.578 | 1.00 | 57.26 |
| ATOM | 19324 | C | VAL | 2885 | 56.079 | 26.632 | 16.519 | 1.00 | 58.32 |
| ATOM | 19325 | O | VAL | 2885 | 56.046 | 25.867 | 15.553 | 1.00 | 58.54 |
| ATOM | 19326 | N | GLU | 2886 | 57.108 | 26.701 | 17.358 | 1.00 | 58.72 |
| ATOM | 19327 | CA | GLU | 2886 | 58.300 | 25.876 | 17.194 | 1.00 | 58.51 |
| ATOM | 19328 | CB | GLU | 2886 | 59.282 | 26.138 | 18.341 | 1.00 | 59.10 |
| ATOM | 19329 | CG | GLU | 2886 | 59.379 | 27.602 | 18.763 | 1.00 | 59.54 |
| ATOM | 19330 | CD | GLU | 2886 | 59.529 | 28.549 | 17.586 | 1.00 | 59.84 |
| ATOM | 19331 | OE1 | GLU | 2886 | 60.465 | 28.357 | 16.779 | 1.00 | 60.96 |
| ATOM | 19332 | OE2 | GLU | 2886 | 58.711 | 29.488 | 17.473 | 1.00 | 59.20 |
| ATOM | 19333 | C | GLU | 2886 | 57.937 | 24.394 | 17.164 | 1.00 | 58.20 |
| ATOM | 19334 | O | GLU | 2886 | 58.373 | 23.650 | 16.283 | 1.00 | 57.63 |
| ATOM | 19335 | N | LEU | 2887 | 57.131 | 23.975 | 18.134 | 1.00 | 57.62 |
| ATOM | 19336 | CA | LEU | 2887 | 56.706 | 22.586 | 18.235 | 1.00 | 57.41 |
| ATOM | 19337 | CB | LEU | 2887 | 55.937 | 22.371 | 19.540 | 1.00 | 56.08 |
| ATOM | 19338 | CG | LEU | 2887 | 56.331 | 21.145 | 20.368 | 1.00 | 55.97 |
| ATOM | 19339 | CD1 | LEU | 2887 | 55.545 | 21.140 | 21.668 | 1.00 | 55.92 |
| ATOM | 19340 | CD2 | LEU | 2887 | 56.075 | 19.875 | 19.578 | 1.00 | 55.54 |
| ATOM | 19341 | C | LEU | 2887 | 55.833 | 22.193 | 17.046 | 1.00 | 57.47 |
| ATOM | 19342 | O | LEU | 2887 | 55.948 | 21.086 | 16.519 | 1.00 | 57.79 |
| ATOM | 19343 | N | ALA | 2888 | 54.962 | 23.106 | 16.628 | 1.00 | 57.65 |
| ATOM | 19344 | CA | ALA | 2888 | 54.071 | 22.861 | 15.500 | 1.00 | 57.47 |
| ATOM | 19345 | CB | ALA | 2888 | 53.138 | 24.051 | 15.306 | 1.00 | 57.84 |
| ATOM | 19346 | C | ALA | 2888 | 54.859 | 22.604 | 14.219 | 1.00 | 57.89 |
| ATOM | 19347 | O | ALA | 2888 | 54.491 | 21.751 | 13.411 | 1.00 | 57.53 |
| ATOM | 19348 | N | LYS | 2889 | 55.944 | 23.352 | 14.040 | 1.00 | 57.61 |
| ATOM | 19349 | CA | LYS | 2889 | 56.790 | 23.211 | 12.862 | 1.00 | 57.71 |
| ATOM | 19350 | CB | LYS | 2889 | 57.952 | 24.205 | 12.929 | 1.00 | 58.61 |
| ATOM | 19351 | CG | LYS | 2889 | 57.518 | 25.662 | 12.982 | 1.00 | 60.67 |
| ATOM | 19352 | CD | LYS | 2889 | 58.694 | 26.590 | 13.263 | 1.00 | 62.37 |
| ATOM | 19353 | CE | LYS | 2889 | 58.243 | 28.043 | 13.347 | 1.00 | 62.84 |
| ATOM | 19354 | NZ | LYS | 2889 | 59.353 | 28.959 | 13.729 | 1.00 | 63.21 |
| ATOM | 19355 | C | LYS | 2889 | 57.337 | 21.794 | 12.763 | 1.00 | 57.10 |
| ATOM | 19356 | O | LYS | 2889 | 57.302 | 21.180 | 11.696 | 1.00 | 57.17 |
| ATOM | 19357 | N | ARG | 2890 | 57.840 | 21.281 | 13.881 | 1.00 | 56.24 |
| ATOM | 19358 | CA | ARG | 2890 | 58.401 | 19.936 | 13.923 | 1.00 | 55.63 |
| ATOM | 19359 | CB | ARG | 2890 | 58.973 | 19.639 | 15.311 | 1.00 | 56.65 |
| ATOM | 19360 | CG | ARG | 2890 | 60.157 | 20.501 | 15.697 | 1.00 | 58.26 |
| ATOM | 19361 | CD | ARG | 2890 | 60.844 | 19.945 | 16.934 | 1.00 | 60.00 |
| ATOM | 19362 | NE | ARG | 2890 | 59.947 | 19.894 | 18.084 | 1.00 | 61.53 |
| ATOM | 19363 | CZ | ARG | 2890 | 60.260 | 19.344 | 19.254 | 1.00 | 62.29 |
| ATOM | 19364 | NH1 | ARG | 2890 | 61.451 | 18.791 | 19.433 | 1.00 | 62.17 |
| ATOM | 19365 | NH2 | ARG | 2890 | 59.381 | 19.349 | 20.246 | 1.00 | 63.05 |
| ATOM | 19366 | C | ARG | 2890 | 57.364 | 18.880 | 13.573 | 1.00 | 54.19 |
| ATOM | 19367 | O | ARG | 2890 | 57.502 | 18.162 | 12.584 | 1.00 | 53.84 |
| ATOM | 19368 | N | ILE | 2891 | 56.329 | 18.790 | 14.401 | 1.00 | 52.97 |
| ATOM | 19369 | CA | ILE | 2891 | 55.256 | 17.826 | 14.202 | 1.00 | 51.09 |
| ATOM | 19370 | CB | ILE | 2891 | 54.099 | 18.088 | 15.187 | 1.00 | 51.37 |
| ATOM | 19371 | CG2 | ILE | 2891 | 52.970 | 17.095 | 14.947 | 1.00 | 50.95 |
| ATOM | 19372 | CG1 | ILE | 2891 | 54.613 | 17.981 | 16.626 | 1.00 | 50.98 |
| ATOM | 19373 | CD1 | ILE | 2891 | 53.598 | 18.382 | 17.676 | 1.00 | 50.45 |
| ATOM | 19374 | C | ILE | 2891 | 54.712 | 17.874 | 12.777 | 1.00 | 50.48 |
| ATOM | 19375 | O | ILE | 2891 | 54.621 | 16.845 | 12.108 | 1.00 | 50.45 |
| ATOM | 19376 | N | THR | 2892 | 54.358 | 19.069 | 12.316 | 1.00 | 49.45 |
| ATOM | 19377 | CA | THR | 2892 | 53.816 | 19.230 | 10.973 | 1.00 | 49.23 |
| ATOM | 19378 | CB | THR | 2892 | 53.557 | 20.713 | 10.637 | 1.00 | 48.82 |
| ATOM | 19379 | OG1 | THR | 2892 | 52.662 | 21.275 | 11.603 | 1.00 | 49.13 |
| ATOM | 19380 | CG2 | THR | 2892 | 52.937 | 20.843 | 9.249 | 1.00 | 48.85 |
| ATOM | 19381 | C | THR | 2892 | 54.740 | 18.649 | 9.908 | 1.00 | 49.30 |
| ATOM | 19382 | O | THR | 2892 | 54.280 | 18.005 | 8.965 | 1.00 | 48.82 |
| ATOM | 19383 | N | GLU | 2893 | 56.041 | 18.878 | 10.056 | 1.00 | 49.78 |
| ATOM | 19384 | CA | GLU | 2893 | 57.007 | 18.369 | 9.088 | 1.00 | 50.32 |
| ATOM | 19385 | CB | GLU | 2893 | 58.245 | 19.268 | 9.043 | 1.00 | 51.93 |
| ATOM | 19386 | CG | GLU | 2893 | 57.979 | 20.650 | 8.460 | 1.00 | 54.14 |
| ATOM | 19387 | CD | GLU | 2893 | 59.255 | 21.412 | 8.163 | 1.00 | 55.53 |
| ATOM | 19388 | OE1 | GLU | 2893 | 60.036 | 21.666 | 9.106 | 1.00 | 55.46 |
| ATOM | 19389 | OE2 | GLU | 2893 | 59.477 | 21.757 | 6.982 | 1.00 | 56.24 |
| ATOM | 19390 | C | GLU | 2893 | 57.423 | 16.933 | 9.381 | 1.00 | 49.42 |
| ATOM | 19391 | O | GLU | 2893 | 57.953 | 16.243 | 8.509 | 1.00 | 49.42 |
| ATOM | 19392 | N | ALA | 2894 | 57.174 | 16.487 | 10.609 | 1.00 | 48.11 |
| ATOM | 19393 | CA | ALA | 2894 | 57.518 | 15.131 | 11.021 | 1.00 | 46.74 |
| ATOM | 19394 | CB | ALA | 2894 | 57.718 | 15.079 | 12.531 | 1.00 | 46.75 |
| ATOM | 19395 | C | ALA | 2894 | 56.438 | 14.135 | 10.604 | 1.00 | 45.44 |
| ATOM | 19396 | O | ALA | 2894 | 56.721 | 12.959 | 10.379 | 1.00 | 45.28 |
| ATOM | 19397 | N | LEU | 2895 | 55.200 | 14.608 | 10.500 | 1.00 | 44.09 |
| ATOM | 19398 | CA | LEU | 2895 | 54.097 | 13.740 | 10.112 | 1.00 | 42.66 |
| ATOM | 19399 | CB | LEU | 2895 | 52.845 | 14.067 | 10.932 | 1.00 | 43.61 |
| ATOM | 19400 | CG | LEU | 2895 | 52.966 | 13.940 | 12.450 | 1.00 | 43.86 |
| ATOM | 19401 | CD1 | LEU | 2895 | 51.651 | 14.323 | 13.105 | 1.00 | 44.30 |
| ATOM | 19402 | CD2 | LEU | 2895 | 53.350 | 12.520 | 12.814 | 1.00 | 45.06 |
| ATOM | 19403 | C | LEU | 2895 | 53.777 | 13.884 | 8.631 | 1.00 | 41.50 |
| ATOM | 19404 | O | LEU | 2895 | 53.887 | 14.973 | 8.067 | 1.00 | 41.74 |
| ATOM | 19405 | N | ALA | 2896 | 53.384 | 12.776 | 8.009 | 1.00 | 39.31 |
| ATOM | 19406 | CA | ALA | 2896 | 53.020 | 12.769 | 6.597 | 1.00 | 37.81 |
| ATOM | 19407 | CB | ALA | 2896 | 53.230 | 11.380 | 6.003 | 1.00 | 37.19 |
| ATOM | 19408 | C | ALA | 2896 | 51.558 | 13.178 | 6.468 | 1.00 | 37.41 |
| ATOM | 19409 | O | ALA | 2896 | 51.144 | 13.749 | 5.458 | 1.00 | 35.35 |
| ATOM | 19410 | N | ILE | 2897 | 50.776 | 12.874 | 7.499 | 1.00 | 37.72 |
| ATOM | 19411 | CA | ILE | 2897 | 49.359 | 13.222 | 7.510 | 1.00 | 37.82 |
| ATOM | 19412 | CB | ILE | 2897 | 48.589 | 12.404 | 8.572 | 1.00 | 38.19 |
| ATOM | 19413 | CG2 | ILE | 2897 | 48.562 | 10.938 | 8.182 | 1.00 | 35.52 |
| ATOM | 19414 | CG1 | ILE | 2897 | 49.237 | 12.602 | 9.947 | 1.00 | 36.77 |
| ATOM | 19415 | CD1 | ILE | 2897 | 48.490 | 11.937 | 11.083 | 1.00 | 38.83 |
| ATOM | 19416 | C | ILE | 2897 | 49.202 | 14.706 | 7.825 | 1.00 | 38.40 |
| ATOM | 19417 | O | ILE | 2897 | 49.987 | 15.274 | 8.585 | 1.00 | 37.92 |
| ATOM | 19418 | N | PRO | 2898 | 48.180 | 15.354 | 7.247 | 1.00 | 38.51 |
| ATOM | 19419 | CD | PRO | 2898 | 47.116 | 14.796 | 6.397 | 1.00 | 38.81 |
| ATOM | 19420 | CA | PRO | 2898 | 47.953 | 16.781 | 7.492 | 1.00 | 39.39 |
| ATOM | 19421 | CB | PRO | 2898 | 46.729 | 17.090 | 6.629 | 1.00 | 39.31 |
| ATOM | 19422 | CG | PRO | 2898 | 46.001 | 15.787 | 6.601 | 1.00 | 40.01 |
| ATOM | 19423 | C | PRO | 2898 | 47.730 | 17.097 | 8.968 | 1.00 | 40.59 |
| ATOM | 19424 | O | PRO | 2898 | 46.998 | 16.391 | 9.665 | 1.00 | 40.56 |
| ATOM | 19425 | N | VAL | 2899 | 48.375 | 18.158 | 9.441 | 1.00 | 40.40 |
| ATOM | 19426 | CA | VAL | 2899 | 48.252 | 18.569 | 10.832 | 1.00 | 40.89 |
| ATOM | 19427 | CB | VAL | 2899 | 49.641 | 18.739 | 11.484 | 1.00 | 41.08 |
| ATOM | 19428 | CG1 | VAL | 2899 | 49.487 | 19.099 | 12.952 | 1.00 | 40.58 |
| ATOM | 19429 | CG2 | VAL | 2899 | 50.442 | 17.457 | 11.334 | 1.00 | 41.04 |
| ATOM | 19430 | C | VAL | 2899 | 47.489 | 19.882 | 10.942 | 1.00 | 41.61 |
| ATOM | 19431 | O | VAL | 2899 | 48.002 | 20.946 | 10.594 | 1.00 | 42.66 |
| ATOM | 19432 | N | ILE | 2900 | 46.254 | 19.797 | 11.424 | 1.00 | 41.85 |
| ATOM | 19433 | CA | ILE | 2900 | 45.409 | 20.973 | 11.588 | 1.00 | 40.80 |
| ATOM | 19434 | CB | ILE | 2900 | 43.918 | 20.589 | 11.510 | 1.00 | 40.42 |
| ATOM | 19435 | CG2 | ILE | 2900 | 43.046 | 21.818 | 11.739 | 1.00 | 40.48 |
| ATOM | 19436 | CG1 | ILE | 2900 | 43.627 | 19.954 | 10.146 | 1.00 | 40.12 |
| ATOM | 19437 | CD1 | ILE | 2900 | 42.223 | 19.401 | 10.000 | 1.00 | 38.79 |
| ATOM | 19438 | C | ILE | 2900 | 45.701 | 21.599 | 12.943 | 1.00 | 40.37 |
| ATOM | 19439 | O | ILE | 2900 | 45.760 | 20.902 | 13.955 | 1.00 | 40.86 |
| ATOM | 19440 | N | GLY | 2901 | 45.892 | 22.913 | 12.964 | 1.00 | 40.33 |
| ATOM | 19441 | CA | GLY | 2901 | 46.189 | 23.576 | 14.219 | 1.00 | 39.83 |
| ATOM | 19442 | C | GLY | 2901 | 45.312 | 24.771 | 14.534 | 1.00 | 39.82 |
| ATOM | 19443 | O | GLY | 2901 | 44.743 | 25.400 | 13.644 | 1.00 | 39.28 |
| ATOM | 19444 | N | ILE | 2902 | 45.198 | 25.071 | 15.822 | 1.00 | 40.24 |
| ATOM | 19445 | CA | ILE | 2902 | 44.414 | 26.203 | 16.287 | 1.00 | 41.59 |
| ATOM | 19446 | CB | ILE | 2902 | 43.043 | 25.758 | 16.859 | 1.00 | 41.56 |
| ATOM | 19447 | CG2 | ILE | 2902 | 43.232 | 24.634 | 17.871 | 1.00 | 39.96 |
| ATOM | 19448 | CG1 | ILE | 2902 | 42.333 | 26.957 | 17.490 | 1.00 | 42.23 |
| ATOM | 19449 | CD1 | ILE | 2902 | 40.886 | 26.696 | 17.857 | 1.00 | 42.44 |
| ATOM | 19450 | C | ILE | 2902 | 45.225 | 26.913 | 17.364 | 1.00 | 42.95 |
| ATOM | 19451 | O | ILE | 2902 | 45.405 | 26.397 | 18.468 | 1.00 | 41.71 |
| ATOM | 19452 | N | GLY | 2903 | 45.729 | 28.096 | 17.027 | 1.00 | 44.58 |
| ATOM | 19453 | CA | GLY | 2903 | 46.535 | 28.845 | 17.972 | 1.00 | 45.63 |
| ATOM | 19454 | C | GLY | 2903 | 47.918 | 28.234 | 18.068 | 1.00 | 46.17 |
| ATOM | 19455 | O | GLY | 2903 | 48.531 | 28.218 | 19.136 | 1.00 | 46.79 |
| ATOM | 19456 | N | ALA | 2904 | 48.407 | 27.722 | 16.942 | 1.00 | 46.61 |
| ATOM | 19457 | CA | ALA | 2904 | 49.725 | 27.102 | 16.887 | 1.00 | 46.93 |
| ATOM | 19458 | CB | ALA | 2904 | 49.584 | 25.594 | 16.723 | 1.00 | 46.40 |
| ATOM | 19459 | C | ALA | 2904 | 50.565 | 27.674 | 15.749 | 1.00 | 46.66 |
| ATOM | 19460 | O | ALA | 2904 | 51.652 | 27.174 | 15.461 | 1.00 | 46.67 |
| ATOM | 19461 | N | GLY | 2905 | 50.056 | 28.718 | 15.100 | 1.00 | 47.11 |
| ATOM | 19462 | CA | GLY | 2905 | 50.786 | 29.331 | 14.004 | 1.00 | 47.45 |
| ATOM | 19463 | C | GLY | 2905 | 50.290 | 28.905 | 12.634 | 1.00 | 48.01 |
| ATOM | 19464 | O | GLY | 2905 | 49.434 | 28.027 | 12.517 | 1.00 | 48.29 |
| ATOM | 19465 | N | ASN | 2906 | 50.832 | 29.528 | 11.591 | 1.00 | 47.47 |
| ATOM | 19466 | CA | ASN | 2906 | 50.439 | 29.213 | 10.221 | 1.00 | 47.27 |
| ATOM | 19467 | CB | ASN | 2906 | 50.423 | 30.488 | 9.373 | 1.00 | 46.79 |
| ATOM | 19468 | CG | ASN | 2906 | 51.771 | 31.187 | 9.346 | 1.00 | 46.59 |
| ATOM | 19469 | OD1 | ASN | 2906 | 51.940 | 32.206 | 8.674 | 1.00 | 47.15 |
| ATOM | 19470 | ND2 | ASN | 2906 | 52.736 | 30.644 | 10.079 | 1.00 | 45.79 |
| ATOM | 19471 | C | ASN | 2906 | 51.374 | 28.190 | 9.585 | 1.00 | 47.11 |
| ATOM | 19472 | O | ASN | 2906 | 51.368 | 28.002 | 8.366 | 1.00 | 46.64 |
| ATOM | 19473 | N | VAL | 2907 | 52.173 | 27.530 | 10.418 | 1.00 | 47.09 |
| ATOM | 19474 | CA | VAL | 2907 | 53.118 | 26.526 | 9.941 | 1.00 | 47.56 |
| ATOM | 19475 | CB | VAL | 2907 | 54.234 | 26.269 | 10.980 | 1.00 | 48.05 |
| ATOM | 19476 | CG1 | VAL | 2907 | 55.286 | 25.336 | 10.394 | 1.00 | 49.38 |
| ATOM | 19477 | CG2 | VAL | 2907 | 54.866 | 27.584 | 11.404 | 1.00 | 48.66 |
| ATOM | 19478 | C | VAL | 2907 | 52.415 | 25.204 | 9.645 | 1.00 | 47.40 |
| ATOM | 19479 | O | VAL | 2907 | 52.875 | 24.419 | 8.816 | 1.00 | 46.82 |
| ATOM | 19480 | N | THR | 2908 | 51.299 | 24.965 | 10.326 | 1.00 | 46.84 |
| ATOM | 19481 | CA | THR | 2908 | 50.538 | 23.737 | 10.134 | 1.00 | 46.83 |
| ATOM | 19482 | CB | THR | 2908 | 49.387 | 23.644 | 11.145 | 1.00 | 46.34 |
| ATOM | 19483 | OG1 | THR | 2908 | 48.608 | 24.845 | 11.091 | 1.00 | 47.58 |
| ATOM | 19484 | CG2 | THR | 2908 | 49.932 | 23.460 | 12.550 | 1.00 | 45.22 |
| ATOM | 19485 | C | THR | 2908 | 49.966 | 23.646 | 8.722 | 1.00 | 47.30 |
| ATOM | 19486 | O | THR | 2908 | 49.862 | 24.649 | 8.013 | 1.00 | 47.83 |
| ATOM | 19487 | N | ASP | 2909 | 49.598 | 22.436 | 8.318 | 1.00 | 46.81 |
| ATOM | 19488 | CA | ASP | 2909 | 49.047 | 22.204 | 6.989 | 1.00 | 47.02 |
| ATOM | 19489 | CB | ASP | 2909 | 48.974 | 20.700 | 6.718 | 1.00 | 47.68 |
| ATOM | 19490 | CG | ASP | 2909 | 50.313 | 20.009 | 6.906 | 1.00 | 48.92 |
| ATOM | 19491 | OD1 | ASP | 2909 | 51.248 | 20.299 | 6.127 | 1.00 | 49.06 |
| ATOM | 19492 | OD2 | ASP | 2909 | 50.432 | 19.182 | 7.836 | 1.00 | 47.98 |
| ATOM | 19493 | C | ASP | 2909 | 47.666 | 22.831 | 6.832 | 1.00 | 47.23 |
| ATOM | 19494 | O | ASP | 2909 | 47.219 | 23.110 | 5.717 | 1.00 | 46.70 |
| ATOM | 19495 | N | GLY | 2910 | 46.995 | 23.057 | 7.956 | 1.00 | 46.85 |
| ATOM | 19496 | CA | GLY | 2910 | 45.672 | 23.650 | 7.914 | 1.00 | 46.10 |
| ATOM | 19497 | C | GLY | 2910 | 45.335 | 24.437 | 9.163 | 1.00 | 45.53 |
| ATOM | 19498 | O | GLY | 2910 | 46.113 | 24.473 | 10.115 | 1.00 | 44.71 |
| ATOM | 19499 | N | GLN | 2911 | 44.166 | 25.067 | 9.157 | 1.00 | 45.89 |
| ATOM | 19500 | CA | GLN | 2911 | 43.712 | 25.863 | 10.292 | 1.00 | 46.51 |
| ATOM | 19501 | CB | GLN | 2911 | 43.873 | 27.357 | 9.990 | 1.00 | 46.39 |
| ATOM | 19502 | CG | GLN | 2911 | 45.315 | 27.843 | 9.878 | 1.00 | 44.82 |
| ATOM | 19503 | CD | GLN | 2911 | 46.100 | 27.649 | 11.162 | 1.00 | 44.00 |
| ATOM | 19504 | OE1 | GLN | 2911 | 45.634 | 27.997 | 12.246 | 1.00 | 43.65 |
| ATOM | 19505 | NE2 | GLN | 2911 | 47.305 | 27.103 | 11.043 | 1.00 | 44.12 |
| ATOM | 19506 | C | GLN | 2911 | 42.250 | 25.577 | 10.619 | 1.00 | 47.63 |
| ATOM | 19507 | O | GLN | 2911 | 41.487 | 25.114 | 9.771 | 1.00 | 46.88 |
| ATOM | 19508 | N | ILE | 2912 | 41.867 | 25.853 | 11.860 | 1.00 | 48.52 |
| ATOM | 19509 | CA | ILE | 2912 | 40.492 | 25.652 | 12.290 | 1.00 | 50.21 |
| ATOM | 19510 | CB | ILE | 2912 | 40.320 | 24.298 | 13.023 | 1.00 | 50.95 |
| ATOM | 19511 | CG2 | ILE | 2912 | 41.195 | 24.257 | 14.270 | 1.00 | 51.75 |
| ATOM | 19512 | CG1 | ILE | 2912 | 38.849 | 24.088 | 13.376 | 1.00 | 51.34 |
| ATOM | 19513 | CD1 | ILE | 2912 | 38.484 | 22.641 | 13.645 | 1.00 | 52.84 |
| ATOM | 19514 | C | ILE | 2912 | 40.093 | 26.806 | 13.205 | 1.00 | 50.85 |
| ATOM | 19515 | O | ILE | 2912 | 40.870 | 27.222 | 14.066 | 1.00 | 51.15 |
| ATOM | 19516 | N | LEU | 2913 | 38.889 | 27.334 | 13.005 | 1.00 | 51.43 |
| ATOM | 19517 | CA | LEU | 2913 | 38.417 | 28.456 | 13.808 | 1.00 | 51.62 |
| ATOM | 19518 | CB | LEU | 2913 | 38.566 | 29.760 | 13.012 | 1.00 | 52.42 |
| ATOM | 19519 | CG | LEU | 2913 | 39.031 | 31.024 | 13.747 | 1.00 | 53.65 |
| ATOM | 19520 | CD1 | LEU | 2913 | 39.235 | 32.140 | 12.739 | 1.00 | 53.91 |
| ATOM | 19521 | CD2 | LEU | 2913 | 38.016 | 31.436 | 14.802 | 1.00 | 54.69 |
| ATOM | 19522 | C | LEU | 2913 | 36.964 | 28.277 | 14.240 | 1.00 | 50.63 |
| ATOM | 19523 | O | LEU | 2913 | 36.170 | 27.629 | 13.553 | 1.00 | 50.30 |
| ATOM | 19524 | N | VAL | 2914 | 36.627 | 28.853 | 15.389 | 1.00 | 49.32 |
| ATOM | 19525 | CA | VAL | 2914 | 35.274 | 28.778 | 15.922 | 1.00 | 47.68 |
| ATOM | 19526 | CB | VAL | 2914 | 35.275 | 28.908 | 17.455 | 1.00 | 48.06 |
| ATOM | 19527 | CG1 | VAL | 2914 | 33.877 | 28.658 | 18.003 | 1.00 | 49.28 |
| ATOM | 19528 | CG2 | VAL | 2914 | 36.268 | 27.930 | 18.050 | 1.00 | 47.75 |
| ATOM | 19529 | C | VAL | 2914 | 34.463 | 29.920 | 15.324 | 1.00 | 46.26 |
| ATOM | 19530 | O | VAL | 2914 | 34.721 | 31.092 | 15.601 | 1.00 | 45.66 |
| ATOM | 19531 | N | MET | 2915 | 33.486 | 29.570 | 14.496 | 1.00 | 43.97 |
| ATOM | 19532 | CA | MET | 2915 | 32.649 | 30.563 | 13.839 | 1.00 | 42.92 |
| ATOM | 19533 | CB | MET | 2915 | 31.500 | 29.873 | 13.106 | 1.00 | 40.64 |
| ATOM | 19534 | CG | MET | 2915 | 30.616 | 29.023 | 14.000 | 1.00 | 38.03 |
| ATOM | 19535 | SD | MET | 2915 | 28.938 | 28.996 | 13.368 | 1.00 | 33.08 |
| ATOM | 19536 | CE | MET | 2915 | 28.260 | 30.449 | 14.185 | 1.00 | 34.77 |
| ATOM | 19537 | C | MET | 2915 | 32.087 | 31.596 | 14.813 | 1.00 | 42.94 |
| ATOM | 19538 | O | MET | 2915 | 31.937 | 32.766 | 14.468 | 1.00 | 43.50 |
| ATOM | 19539 | N | HIS | 2916 | 31.778 | 31.156 | 16.028 | 1.00 | 43.84 |
| ATOM | 19540 | CA | HIS | 2916 | 31.228 | 32.041 | 17.047 | 1.00 | 43.99 |
| ATOM | 19541 | CB | HIS | 2916 | 30.839 | 31.221 | 18.279 | 1.00 | 43.48 |
| ATOM | 19542 | CG | HIS | 2916 | 29.606 | 30.396 | 18.080 | 1.00 | 42.85 |
| ATOM | 19543 | CD2 | HIS | 2916 | 29.446 | 29.097 | 17.733 | 1.00 | 42.66 |
| ATOM | 19544 | ND1 | HIS | 2916 | 28.336 | 30.925 | 18.176 | 1.00 | 42.76 |
| ATOM | 19545 | CE1 | HIS | 2916 | 27.448 | 29.988 | 17.897 | 1.00 | 42.12 |
| ATOM | 19546 | NE2 | HIS | 2916 | 28.095 | 28.870 | 17.624 | 1.00 | 42.80 |
| ATOM | 19547 | C | HIS | 2916 | 32.192 | 33.165 | 17.424 | 1.00 | 45.02 |
| ATOM | 19548 | O | HIS | 2916 | 31.772 | 34.232 | 17.871 | 1.00 | 43.97 |
| ATOM | 19549 | N | ASP | 2917 | 33.485 | 32.924 | 17.238 | 1.00 | 46.22 |
| ATOM | 19550 | CA | ASP | 2917 | 34.493 | 33.931 | 17.539 | 1.00 | 47.60 |
| ATOM | 19551 | CB | ASP | 2917 | 35.702 | 33.296 | 18.227 | 1.00 | 48.54 |
| ATOM | 19552 | CG | ASP | 2917 | 35.463 | 33.045 | 19.699 | 1.00 | 49.88 |
| ATOM | 19553 | OD1 | ASP | 2917 | 35.083 | 34.002 | 20.404 | 1.00 | 51.64 |
| ATOM | 19554 | OD2 | ASP | 2917 | 35.658 | 31.901 | 20.156 | 1.00 | 51.53 |
| ATOM | 19555 | C | ASP | 2917 | 34.938 | 34.638 | 16.266 | 1.00 | 47.64 |
| ATOM | 19556 | O | ASP | 2917 | 35.581 | 35.685 | 16.317 | 1.00 | 49.23 |
| ATOM | 19557 | N | ALA | 2918 | 34.587 | 34.060 | 15.123 | 1.00 | 47.15 |
| ATOM | 19558 | CA | ALA | 2918 | 34.944 | 34.631 | 13.833 | 1.00 | 46.36 |
| ATOM | 19559 | CB | ALA | 2918 | 34.938 | 33.551 | 12.764 | 1.00 | 45.96 |
| ATOM | 19560 | C | ALA | 2918 | 33.965 | 35.736 | 13.463 | 1.00 | 46.60 |
| ATOM | 19561 | O | ALA | 2918 | 34.275 | 36.607 | 12.651 | 1.00 | 45.86 |
| ATOM | 19562 | N | PHE | 2919 | 32.780 | 35.696 | 14.063 | 1.00 | 45.95 |
| ATOM | 19563 | CA | PHE | 2919 | 31.762 | 36.699 | 13.794 | 1.00 | 46.21 |
| ATOM | 19564 | CB | PHE | 2919 | 30.492 | 36.027 | 13.274 | 1.00 | 47.19 |
| ATOM | 19565 | CG | PHE | 2919 | 30.738 | 35.054 | 12.157 | 1.00 | 48.64 |
| ATOM | 19566 | CD1 | PHE | 2919 | 31.476 | 35.427 | 11.038 | 1.00 | 49.33 |
| ATOM | 19567 | CD2 | PHE | 2919 | 30.224 | 33.764 | 12.219 | 1.00 | 49.14 |
| ATOM | 19568 | CE1 | PHE | 2919 | 31.698 | 34.527 | 9.996 | 1.00 | 49.77 |
| ATOM | 19569 | CE2 | PHE | 2919 | 30.440 | 32.857 | 11.183 | 1.00 | 49.47 |
| ATOM | 19570 | CZ | PHE | 2919 | 31.177 | 33.239 | 10.070 | 1.00 | 49.73 |
| ATOM | 19571 | C | PHE | 2919 | 31.450 | 37.498 | 15.051 | 1.00 | 45.77 |
| ATOM | 19572 | O | PHE | 2919 | 30.435 | 38.193 | 15.122 | 1.00 | 45.54 |
| ATOM | 19573 | N | GLY | 2920 | 32.332 | 37.394 | 16.041 | 1.00 | 45.81 |
| ATOM | 19574 | CA | GLY | 2920 | 32.142 | 38.112 | 17.289 | 1.00 | 46.37 |
| ATOM | 19575 | C | GLY | 2920 | 30.782 | 37.867 | 17.909 | 1.00 | 46.48 |
| ATOM | 19576 | O | GLY | 2920 | 30.207 | 38.760 | 18.530 | 1.00 | 45.85 |
| ATOM | 19577 | N | ILE | 2921 | 30.268 | 36.653 | 17.741 | 1.00 | 46.20 |
| ATOM | 19578 | CA | ILE | 2921 | 28.965 | 36.290 | 18.286 | 1.00 | 47.25 |
| ATOM | 19579 | CB | ILE | 2921 | 28.450 | 34.974 | 17.665 | 1.00 | 46.36 |
| ATOM | 19580 | CG2 | ILE | 2921 | 27.066 | 34.651 | 18.210 | 1.00 | 46.13 |
| ATOM | 19581 | CG1 | ILE | 2921 | 28.410 | 35.101 | 16.140 | 1.00 | 46.06 |
| ATOM | 19582 | CD1 | ILE | 2921 | 28.049 | 33.815 | 15.420 | 1.00 | 45.17 |
| ATOM | 19583 | C | ILE | 2921 | 29.040 | 36.120 | 19.801 | 1.00 | 48.32 |
| ATOM | 19584 | O | ILE | 2921 | 28.137 | 36.534 | 20.526 | 1.00 | 47.30 |
| ATOM | 19585 | N | THR | 2922 | 30.126 | 35.515 | 20.271 | 1.00 | 50.35 |
| ATOM | 19586 | CA | THR | 2922 | 30.316 | 35.285 | 21.696 | 1.00 | 53.99 |
| ATOM | 19587 | CB | THR | 2922 | 31.599 | 34.487 | 21.960 | 1.00 | 53.75 |
| ATOM | 19588 | OG1 | THR | 2922 | 32.721 | 35.209 | 21.442 | 1.00 | 55.46 |
| ATOM | 19589 | CG2 | THR | 2922 | 31.524 | 33.127 | 21.287 | 1.00 | 53.60 |
| ATOM | 19590 | C | THR | 2922 | 30.394 | 36.593 | 22.476 | 1.00 | 56.52 |
| ATOM | 19591 | O | THR | 2922 | 30.209 | 37.673 | 21.915 | 1.00 | 57.67 |
| ATOM | 19592 | N | GLY | 2923 | 30.672 | 36.483 | 23.772 | 1.00 | 58.60 |
| ATOM | 19593 | CA | GLY | 2923 | 30.768 | 37.657 | 24.622 | 1.00 | 61.66 |
| ATOM | 19594 | C | GLY | 2923 | 31.545 | 38.803 | 24.004 | 1.00 | 63.68 |
| ATOM | 19595 | O | GLY | 2923 | 30.961 | 39.699 | 23.394 | 1.00 | 64.36 |
| ATOM | 19596 | N | GLY | 2924 | 32.864 | 38.779 | 24.162 | 1.00 | 64.67 |
| ATOM | 19597 | CA | GLY | 2924 | 33.688 | 39.835 | 23.607 | 1.00 | 66.50 |
| ATOM | 19598 | C | GLY | 2924 | 35.161 | 39.619 | 23.880 | 1.00 | 67.68 |
| ATOM | 19599 | O | GLY | 2924 | 36.015 | 39.989 | 23.072 | 1.00 | 68.28 |
| ATOM | 19600 | N | HIS | 2925 | 35.461 | 39.018 | 25.026 | 1.00 | 68.21 |
| ATOM | 19601 | CA | HIS | 2925 | 36.839 | 38.748 | 25.410 | 1.00 | 68.98 |
| ATOM | 19602 | CB | HIS | 2925 | 36.922 | 38.531 | 26.924 | 1.00 | 70.40 |
| ATOM | 19603 | CG | HIS | 2925 | 36.513 | 39.726 | 27.730 | 1.00 | 71.99 |
| ATOM | 19604 | CD2 | HIS | 2925 | 35.508 | 39.900 | 28.620 | 1.00 | 72.41 |
| ATOM | 19605 | ND1 | HIS | 2925 | 37.178 | 40.932 | 27.662 | 1.00 | 72.60 |
| ATOM | 19606 | CE1 | HIS | 2925 | 36.601 | 41.797 | 28.477 | 1.00 | 72.46 |
| ATOM | 19607 | NE2 | HIS | 2925 | 35.585 | 41.197 | 29.070 | 1.00 | 72.60 |
| ATOM | 19608 | C | HIS | 2925 | 37.384 | 37.524 | 24.674 | 1.00 | 68.50 |
| ATOM | 19609 | O | HIS | 2925 | 37.745 | 36.524 | 25.294 | 1.00 | 68.44 |
| ATOM | 19610 | N | ILE | 2926 | 37.442 | 37.613 | 23.348 | 1.00 | 67.83 |
| ATOM | 19611 | CA | ILE | 2926 | 37.940 | 36.516 | 22.524 | 1.00 | 67.07 |
| ATOM | 19612 | CB | ILE | 2926 | 37.816 | 36.838 | 21.019 | 1.00 | 67.36 |
| ATOM | 19613 | CG2 | ILE | 2926 | 36.353 | 37.011 | 20.641 | 1.00 | 67.76 |
| ATOM | 19614 | CG1 | ILE | 2926 | 38.613 | 38.102 | 20.691 | 1.00 | 67.33 |
| ATOM | 19615 | CD1 | ILE | 2926 | 38.682 | 38.416 | 19.212 | 1.00 | 67.17 |
| ATOM | 19616 | C | ILE | 2926 | 39.406 | 36.224 | 22.827 | 1.00 | 66.38 |
| ATOM | 19617 | O | ILE | 2926 | 40.136 | 37.094 | 23.300 | 1.00 | 66.76 |
| ATOM | 19618 | N | PRO | 2927 | 39.856 | 34.987 | 22.557 | 1.00 | 65.66 |
| ATOM | 19619 | CD | PRO | 2927 | 39.075 | 33.857 | 22.023 | 1.00 | 65.23 |
| ATOM | 19620 | CA | PRO | 2927 | 41.244 | 34.585 | 22.804 | 1.00 | 64.79 |
| ATOM | 19621 | CB | PRO | 2927 | 41.198 | 33.073 | 22.613 | 1.00 | 65.30 |
| ATOM | 19622 | CG | PRO | 2927 | 40.158 | 32.914 | 21.553 | 1.00 | 65.30 |
| ATOM | 19623 | C | PRO | 2927 | 42.229 | 35.261 | 21.854 | 1.00 | 64.22 |
| ATOM | 19624 | O | PRO | 2927 | 41.838 | 35.812 | 20.824 | 1.00 | 63.39 |
| ATOM | 19625 | N | LYS | 2928 | 43.509 | 35.210 | 22.206 | 1.00 | 63.55 |
| ATOM | 19626 | CA | LYS | 2928 | 44.552 | 35.820 | 21.392 | 1.00 | 63.10 |
| ATOM | 19627 | CB | LYS | 2928 | 45.862 | 35.910 | 22.182 | 1.00 | 63.96 |
| ATOM | 19628 | CG | LYS | 2928 | 45.893 | 36.997 | 23.252 | 1.00 | 64.57 |
| ATOM | 19629 | CD | LYS | 2928 | 44.948 | 36.705 | 24.408 | 1.00 | 65.11 |
| ATOM | 19630 | CE | LYS | 2928 | 45.029 | 37.801 | 25.466 | 1.00 | 65.21 |
| ATOM | 19631 | NZ | LYS | 2928 | 44.169 | 37.518 | 26.651 | 1.00 | 64.88 |
| ATOM | 19632 | C | LYS | 2928 | 44.803 | 35.067 | 20.091 | 1.00 | 62.38 |
| ATOM | 19633 | O | LYS | 2928 | 45.363 | 35.622 | 19.145 | 1.00 | 62.12 |
| ATOM | 19634 | N | PHE | 2929 | 44.387 | 33.806 | 20.043 | 1.00 | 61.22 |
| ATOM | 19635 | CA | PHE | 2929 | 44.586 | 32.993 | 18.850 | 1.00 | 59.91 |
| ATOM | 19636 | CB | PHE | 2929 | 44.905 | 31.550 | 19.248 | 1.00 | 59.84 |
| ATOM | 19637 | CG | PHE | 2929 | 43.882 | 30.926 | 20.154 | 1.00 | 60.10 |
| ATOM | 19638 | CD1 | PHE | 2929 | 42.659 | 30.490 | 19.655 | 1.00 | 60.07 |
| ATOM | 19639 | CD2 | PHE | 2929 | 44.144 | 30.772 | 21.511 | 1.00 | 60.02 |
| ATOM | 19640 | CE1 | PHE | 2929 | 41.711 | 29.907 | 20.494 | 1.00 | 59.46 |
| ATOM | 19641 | CE2 | PHE | 2929 | 43.202 | 30.191 | 22.358 | 1.00 | 60.29 |
| ATOM | 19642 | CZ | PHE | 2929 | 41.983 | 29.758 | 21.848 | 1.00 | 59.76 |
| ATOM | 19643 | C | PHE | 2929 | 43.393 | 33.028 | 17.904 | 1.00 | 59.23 |
| ATOM | 19644 | O | PHE | 2929 | 43.370 | 32.321 | 16.897 | 1.00 | 59.32 |
| ATOM | 19645 | N | ALA | 2930 | 42.409 | 33.863 | 18.227 | 1.00 | 57.98 |
| ATOM | 19646 | CA | ALA | 2930 | 41.212 | 33.995 | 17.404 | 1.00 | 56.99 |
| ATOM | 19647 | CB | ALA | 2930 | 39.967 | 33.889 | 18.273 | 1.00 | 56.84 |
| ATOM | 19648 | C | ALA | 2930 | 41.217 | 35.324 | 16.662 | 1.00 | 56.37 |
| ATOM | 19649 | O | ALA | 2930 | 42.091 | 36.161 | 16.876 | 1.00 | 56.26 |
| ATOM | 19650 | N | LYS | 2931 | 40.236 | 35.517 | 15.788 | 1.00 | 56.19 |
| ATOM | 19651 | CA | LYS | 2931 | 40.140 | 36.753 | 15.022 | 1.00 | 56.47 |
| ATOM | 19652 | CB | LYS | 2931 | 41.140 | 36.734 | 13.862 | 1.00 | 56.43 |
| ATOM | 19653 | CG | LYS | 2931 | 41.165 | 38.013 | 13.037 | 1.00 | 56.84 |
| ATOM | 19654 | CD | LYS | 2931 | 42.212 | 37.935 | 11.939 | 1.00 | 57.32 |
| ATOM | 19655 | CE | LYS | 2931 | 42.283 | 39.226 | 11.137 | 1.00 | 57.79 |
| ATOM | 19656 | NZ | LYS | 2931 | 43.308 | 39.151 | 10.053 | 1.00 | 57.52 |
| ATOM | 19657 | C | LYS | 2931 | 38.730 | 36.963 | 14.481 | 1.00 | 56.42 |
| ATOM | 19658 | O | LYS | 2931 | 38.137 | 36.059 | 13.892 | 1.00 | 56.44 |
| ATOM | 19659 | N | ASN | 2932 | 38.202 | 38.165 | 14.687 | 1.00 | 56.03 |
| ATOM | 19660 | CA | ASN | 2932 | 36.867 | 38.510 | 14.220 | 1.00 | 55.89 |
| ATOM | 19661 | CB | ASN | 2932 | 36.274 | 39.617 | 15.095 | 1.00 | 55.63 |
| ATOM | 19662 | CG | ASN | 2932 | 34.795 | 39.827 | 14.847 | 1.00 | 55.62 |
| ATOM | 19663 | OD1 | ASN | 2932 | 34.324 | 39.747 | 13.711 | 1.00 | 55.34 |
| ATOM | 19664 | ND2 | ASN | 2932 | 34.053 | 40.111 | 15.911 | 1.00 | 55.48 |
| ATOM | 19665 | C | ASN | 2932 | 36.946 | 38.999 | 12.777 | 1.00 | 56.13 |
| ATOM | 19666 | O | ASN | 2932 | 37.269 | 40.160 | 12.527 | 1.00 | 56.78 |
| ATOM | 19667 | N | PHE | 2933 | 36.656 | 38.115 | 11.829 | 1.00 | 56.14 |
| ATOM | 19668 | CA | PHE | 2933 | 36.697 | 38.482 | 10.422 | 1.00 | 57.27 |
| ATOM | 19669 | CB | PHE | 2933 | 36.817 | 37.234 | 9.543 | 1.00 | 57.12 |
| ATOM | 19670 | CG | PHE | 2933 | 38.141 | 36.540 | 9.658 | 1.00 | 57.30 |
| ATOM | 19671 | CD1 | PHE | 2933 | 38.472 | 35.829 | 10.807 | 1.00 | 56.85 |
| ATOM | 19672 | CD2 | PHE | 2933 | 39.073 | 36.621 | 8.627 | 1.00 | 57.13 |
| ATOM | 19673 | CE1 | PHE | 2933 | 39.713 | 35.209 | 10.927 | 1.00 | 57.60 |
| ATOM | 19674 | CE2 | PHE | 2933 | 40.315 | 36.006 | 8.737 | 1.00 | 57.30 |
| ATOM | 19675 | CZ | PHE | 2933 | 40.637 | 35.299 | 9.889 | 1.00 | 57.81 |
| ATOM | 19676 | C | PHE | 2933 | 35.471 | 39.284 | 10.009 | 1.00 | 58.04 |
| ATOM | 19677 | O | PHE | 2933 | 35.464 | 39.925 | 8.957 | 1.00 | 58.08 |
| ATOM | 19678 | N | LEU | 2934 | 34.432 | 39.242 | 10.837 | 1.00 | 59.10 |
| ATOM | 19679 | CA | LEU | 2934 | 33.205 | 39.980 | 10.556 | 1.00 | 59.92 |
| ATOM | 19680 | CB | LEU | 2934 | 32.050 | 39.449 | 11.409 | 1.00 | 58.78 |
| ATOM | 19681 | CG | LEU | 2934 | 30.721 | 40.200 | 11.278 | 1.00 | 58.03 |
| ATOM | 19682 | CD1 | LEU | 2934 | 30.222 | 40.120 | 9.844 | 1.00 | 57.10 |
| ATOM | 19683 | CD2 | LEU | 2934 | 29.698 | 39.610 | 12.232 | 1.00 | 57.76 |
| ATOM | 19684 | C | LEU | 2934 | 33.426 | 41.454 | 10.864 | 1.00 | 61.04 |
| ATOM | 19685 | O | LEU | 2934 | 32.864 | 42.326 | 10.204 | 1.00 | 61.19 |
| ATOM | 19686 | N | ALA | 2935 | 34.247 | 41.721 | 11.874 | 1.00 | 62.38 |
| ATOM | 19687 | CA | ALA | 2935 | 34.552 | 43.086 | 12.274 | 1.00 | 64.22 |
| ATOM | 19688 | CB | ALA | 2935 | 35.328 | 43.085 | 13.583 | 1.00 | 63.92 |
| ATOM | 19689 | C | ALA | 2935 | 35.362 | 43.775 | 11.182 | 1.00 | 65.41 |
| ATOM | 19690 | O | ALA | 2935 | 35.204 | 44.972 | 10.935 | 1.00 | 65.96 |
| ATOM | 19691 | N | GLU | 2936 | 36.229 | 43.005 | 10.529 | 1.00 | 66.66 |
| ATOM | 19692 | CA | GLU | 2936 | 37.070 | 43.523 | 9.454 | 1.00 | 67.61 |
| ATOM | 19693 | CB | GLU | 2936 | 38.167 | 42.511 | 9.105 | 1.00 | 68.36 |
| ATOM | 19694 | CG | GLU | 2936 | 38.961 | 41.982 | 10.294 | 1.00 | 69.12 |
| ATOM | 19695 | CD | GLU | 2936 | 39.699 | 43.070 | 11.051 | 1.00 | 69.89 |
| ATOM | 19696 | OE1 | GLU | 2936 | 39.034 | 43.903 | 11.703 | 1.00 | 70.02 |
| ATOM | 19697 | OE2 | GLU | 2936 | 40.948 | 43.094 | 10.989 | 1.00 | 70.26 |
| ATOM | 19698 | C | GLU | 2936 | 36.216 | 43.788 | 8.217 | 1.00 | 67.80 |
| ATOM | 19699 | O | GLU | 2936 | 36.719 | 44.238 | 7.187 | 1.00 | 67.71 |
| ATOM | 19700 | N | THR | 2937 | 34.923 | 43.497 | 8.328 | 1.00 | 67.74 |
| ATOM | 19701 | CA | THR | 2937 | 33.988 | 43.696 | 7.228 | 1.00 | 67.18 |
| ATOM | 19702 | CB | THR | 2937 | 33.961 | 42.455 | 6.306 | 1.00 | 67.51 |
| ATOM | 19703 | OG1 | TSR | 2937 | 33.149 | 42.726 | 5.157 | 1.00 | 67.78 |
| ATOM | 19704 | CG2 | THR | 2937 | 33.399 | 41.249 | 7.048 | 1.00 | 67.76 |
| ATOM | 19705 | C | THR | 2937 | 32.577 | 43.974 | 7.757 | 1.00 | 66.84 |
| ATOM | 19706 | O | THR | 2937 | 32.410 | 44.481 | 8.869 | 1.00 | 66.83 |
| ATOM | 19707 | N | GLY | 2938 | 31.565 | 43.650 | 6.958 | 1.00 | 66.24 |
| ATOM | 19708 | CA | GLY | 2938 | 30.192 | 43.873 | 7.374 | 1.00 | 65.54 |
| ATOM | 19709 | C | GLY | 2938 | 29.275 | 42.763 | 6.899 | 1.00 | 64.98 |
| ATOM | 19710 | O | GLY | 2938 | 28.054 | 42.842 | 7.043 | 1.00 | 64.61 |
| ATOM | 19711 | N | ASP | 2939 | 29.874 | 41.721 | 6.331 | 1.00 | 64.32 |
| ATOM | 19712 | CA | ASP | 2939 | 29.123 | 40.582 | 5.822 | 1.00 | 63.45 |
| ATOM | 19713 | CB | ASP | 2939 | 29.128 | 40.595 | 4.291 | 1.00 | 64.45 |
| ATOM | 19714 | CG | ASP | 2939 | 28.216 | 39.544 | 3.698 | 1.00 | 65.72 |
| ATOM | 19715 | OD1 | ASP | 2939 | 28.488 | 38.340 | 3.889 | 1.00 | 66.50 |
| ATOM | 19716 | OD2 | ASP | 2939 | 27.222 | 39.924 | 3.042 | 1.00 | 66.35 |
| ATOM | 19717 | C | ASP | 2939 | 29.725 | 39.277 | 6.336 | 1.00 | 62.43 |
| ATOM | 19718 | O | ASP | 2939 | 30.928 | 39.044 | 6.209 | 1.00 | 61.79 |
| ATOM | 19719 | N | ILE | 2940 | 28.878 | 38.433 | 6.920 | 1.00 | 61.10 |
| ATOM | 19720 | CA | ILE | 2940 | 29.310 | 37.148 | 7.463 | 1.00 | 59.37 |
| ATOM | 19721 | CB | ILE | 2940 | 28.105 | 36.342 | 8.007 | 1.00 | 59.30 |
| ATOM | 19722 | CG2 | ILE | 2940 | 28.577 | 34.996 | 8.539 | 1.00 | 59.02 |
| ATOM | 19723 | CG1 | ILE | 2940 | 27.404 | 37.136 | 9.113 | 1.00 | 59.59 |
| ATOM | 19724 | CD1 | ILE | 2940 | 26.152 | 36.472 | 9.656 | 1.00 | 59.03 |
| ATOM | 19725 | C | ILE | 2940 | 30.026 | 36.308 | 6.408 | 1.00 | 58.22 |
| ATOM | 19726 | O | ILE | 2940 | 31.116 | 35.792 | 6.652 | 1.00 | 57.54 |
| ATOM | 19727 | N | ARG | 2941 | 29.407 | 36.171 | 5.240 | 1.00 | 57.25 |
| ATOM | 19728 | CA | ARG | 2941 | 29.991 | 35.395 | 4.154 | 1.00 | 56.77 |
| ATOM | 19729 | CB | ARG | 2941 | 29.033 | 35.348 | 2.963 | 1.00 | 55.80 |
| ATOM | 19730 | CG | ARG | 2941 | 27.778 | 34.525 | 3.206 | 1.00 | 53.72 |
| ATOM | 19731 | CD | ARG | 2941 | 26.838 | 34.590 | 2.013 | 1.00 | 52.37 |
| ATOM | 19732 | NE | ARG | 2941 | 25.691 | 33.698 | 2.165 | 1.00 | 51.33 |
| ATOM | 19733 | CZ | ARG | 2941 | 24.805 | 33.775 | 3.153 | 1.00 | 50.67 |
| ATOM | 19734 | NH1 | ARG | 2941 | 24.927 | 34.706 | 4.089 | 1.00 | 50.47 |
| ATOM | 19735 | NH2 | ARG | 2941 | 23.793 | 32.919 | 3.204 | 1.00 | 50.57 |
| ATOM | 19736 | C | ARG | 2941 | 31.328 | 35.986 | 3.721 | 1.00 | 57.51 |
| ATOM | 19737 | O | ARG | 2941 | 32.278 | 35.254 | 3.441 | 1.00 | 58.07 |
| ATOM | 19738 | N | ALA | 2942 | 31.394 | 37.313 | 3.666 | 1.00 | 57.53 |
| ATOM | 19739 | CA | ALA | 2942 | 32.618 | 38.001 | 3.275 | 1.00 | 57.14 |
| ATOM | 19740 | CB | ALA | 2942 | 32.384 | 39.506 | 3.243 | 1.00 | 57.24 |
| ATOM | 19741 | C | ALA | 2942 | 33.729 | 37.663 | 4.262 | 1.00 | 56.97 |
| ATOM | 19742 | O | ALA | 2942 | 34.882 | 37.475 | 3.874 | 1.00 | 57.28 |
| ATOM | 19743 | N | ALA | 2943 | 33.372 | 37.584 | 5.540 | 1.00 | 56.48 |
| ATOM | 19744 | CA | ALA | 2943 | 34.338 | 37.263 | 6.583 | 1.00 | 56.25 |
| ATOM | 19745 | CB | ALA | 2943 | 33.679 | 37.359 | 7.950 | 1.00 | 56.65 |
| ATOM | 19746 | C | ALA | 2943 | 34.896 | 35.861 | 6.368 | 1.00 | 56.16 |
| ATOM | 19747 | O | ALA | 2943 | 36.044 | 35.583 | 6.712 | 1.00 | 56.09 |
| ATOM | 19748 | N | VAL | 2944 | 34.077 | 34.984 | 5.792 | 1.00 | 56.16 |
| ATOM | 19749 | CA | VAL | 2944 | 34.485 | 33.609 | 5.526 | 1.00 | 55.63 |
| ATOM | 19750 | CB | VAL | 2944 | 33.279 | 32.735 | 5.106 | 1.00 | 56.09 |
| ATOM | 19751 | CG1 | VAL | 2944 | 33.745 | 31.318 | 4.801 | 1.00 | 55.49 |
| ATOM | 19752 | CG2 | VAL | 2944 | 32.234 | 32.722 | 6.213 | 1.00 | 55.66 |
| ATOM | 19753 | C | VAL | 2944 | 35.532 | 33.557 | 4.420 | 1.00 | 55.75 |
| ATOM | 19754 | O | VAL | 2944 | 36.568 | 32.909 | 4.569 | 1.00 | 55.47 |
| ATOM | 19755 | N | ARG | 2945 | 35.257 | 34.238 | 3.311 | 1.00 | 55.89 |
| ATOM | 19756 | CA | ARG | 2945 | 36.188 | 34.264 | 2.189 | 1.00 | 55.76 |
| ATOM | 19757 | CB | ARG | 2945 | 35.622 | 35.094 | 1.034 | 1.00 | 55.60 |
| ATOM | 19758 | CG | ARG | 2945 | 34.413 | 34.471 | 0.356 | 1.00 | 56.71 |
| ATOM | 19759 | CD | ARG | 2945 | 34.101 | 35.175 | −0.956 | 1.00 | 57.24 |
| ATOM | 19760 | NE | ARG | 2945 | 33.775 | 36.585 | −0.762 | 1.00 | 57.48 |
| ATOM | 19761 | CZ | ARG | 2945 | 32.653 | 37.024 | −0.199 | 1.00 | 57.88 |
| ATOM | 19762 | NH1 | ARG | 2945 | 31.740 | 36.163 | 0.228 | 1.00 | 57.99 |
| ATOM | 19763 | NH2 | ARG | 2945 | 32.445 | 38.327 | −0.061 | 1.00 | 57.70 |
| ATOM | 19764 | C | ARG | 2945 | 37.532 | 34.836 | 2.617 | 1.00 | 55.48 |
| ATOM | 19765 | O | ARG | 2945 | 38.585 | 34.335 | 2.216 | 1.00 | 55.77 |
| ATOM | 19766 | N | GLN | 2946 | 37.491 | 35.886 | 3.431 | 1.00 | 54.96 |
| ATOM | 19767 | CA | GLN | 2946 | 38.710 | 36.520 | 3.918 | 1.00 | 54.60 |
| ATOM | 19768 | CB | GLN | 2946 | 38.368 | 37.747 | 4.769 | 1.00 | 55.75 |
| ATOM | 19769 | CG | GLN | 2946 | 39.574 | 38.580 | 5.179 | 1.00 | 57.10 |
| ATOM | 19770 | CD | GLN | 2946 | 39.215 | 39.681 | 6.161 | 1.00 | 58.43 |
| ATOM | 19771 | OE1 | GLN | 2946 | 38.307 | 40.478 | 5.917 | 1.00 | 59.03 |
| ATOM | 19772 | NE2 | GLN | 2946 | 39.932 | 39.733 | 7.279 | 1.00 | 58.79 |
| ATOM | 19773 | C | GLN | 2946 | 39.482 | 35.509 | 4.757 | 1.00 | 53.60 |
| ATOM | 19774 | O | GLN | 2946 | 40.707 | 35.416 | 4.670 | 1.00 | 53.90 |
| ATOM | 19775 | N | TYR | 2947 | 38.752 | 34.754 | 5.571 | 1.00 | 52.23 |
| ATOM | 19776 | CA | TYR | 2947 | 39.350 | 33.736 | 6.426 | 1.00 | 50.90 |
| ATOM | 19777 | CB | TYR | 2947 | 38.267 | 33.086 | 7.291 | 1.00 | 49.81 |
| ATOM | 19778 | CG | TYR | 2947 | 38.721 | 31.849 | 8.030 | 1.00 | 48.86 |
| ATOM | 19779 | CD1 | TYR | 2947 | 39.824 | 31.887 | 8.882 | 1.00 | 48.05 |
| ATOM | 19780 | CE1 | TYR | 2947 | 40.240 | 30.751 | 9.570 | 1.00 | 48.01 |
| ATOM | 19781 | CD2 | TYR | 2947 | 38.042 | 30.639 | 7.884 | 1.00 | 48.25 |
| ATOM | 19782 | CE2 | TYR | 2947 | 38.449 | 29.497 | 8.567 | 1.00 | 47.92 |
| ATOM | 19783 | CZ | TYR | 2947 | 39.548 | 29.559 | 9.408 | 1.00 | 48.00 |
| ATOM | 19784 | OH | TYR | 2947 | 39.957 | 28.434 | 10.087 | 1.00 | 46.78 |
| ATOM | 19785 | C | TYR | 2947 | 40.045 | 32.679 | 5.574 | 1.00 | 50.61 |
| ATOM | 19786 | O | TYR | 2947 | 41.195 | 32.315 | 5.828 | 1.00 | 50.43 |
| ATOM | 19787 | N | MET | 2948 | 39.333 | 32.194 | 4.562 | 1.00 | 50.53 |
| ATOM | 19788 | CA | MET | 2948 | 39.856 | 31.183 | 3.652 | 1.00 | 50.34 |
| ATOM | 19789 | CB | MET | 2948 | 38.785 | 30.803 | 2.627 | 1.00 | 50.02 |
| ATOM | 19790 | CG | MET | 2948 | 37.518 | 30.204 | 3.220 | 1.00 | 47.76 |
| ATOM | 19791 | SD | MET | 2948 | 36.190 | 30.122 | 1.998 | 1.00 | 48.64 |
| ATOM | 19792 | CE | MET | 2948 | 36.932 | 29.063 | 0.769 | 1.00 | 48.77 |
| ATOM | 19793 | C | MET | 2948 | 41.094 | 31.701 | 2.921 | 1.00 | 51.03 |
| ATOM | 19794 | O | MET | 2948 | 42.109 | 31.008 | 2.827 | 1.00 | 51.21 |
| ATOM | 19795 | N | ALA | 2949 | 41.001 | 32.923 | 2.407 | 1.00 | 50.88 |
| ATOM | 19796 | CA | ALA | 2949 | 42.103 | 33.533 | 1.674 | 1.00 | 51.33 |
| ATOM | 19797 | CB | ALA | 2949 | 41.640 | 34.835 | 1.033 | 1.00 | 51.05 |
| ATOM | 19798 | C | ALA | 2949 | 43.328 | 33.789 | 2.547 | 1.00 | 51.80 |
| ATOM | 19799 | O | ALA | 2949 | 44.457 | 33.528 | 2.128 | 1.00 | 52.35 |
| ATOM | 19800 | N | GLU | 2950 | 43.112 | 34.296 | 3.758 | 1.00 | 52.03 |
| ATOM | 19801 | CA | GLU | 2950 | 44.222 | 34.583 | 4.662 | 1.00 | 52.73 |
| ATOM | 19802 | CB | GLU | 2950 | 43.735 | 35.373 | 5.880 | 1.00 | 52.54 |
| ATOM | 19803 | CG | GLU | 2950 | 43.593 | 36.864 | 5.622 | 1.00 | 53.18 |
| ATOM | 19804 | CD | GLU | 2950 | 43.213 | 37.643 | 6.867 | 1.00 | 53.56 |
| ATOM | 19805 | OE1 | GLU | 2950 | 43.847 | 37.429 | 7.923 | 1.00 | 53.35 |
| ATOM | 19806 | OE2 | GLU | 2950 | 42.287 | 38.478 | 6.787 | 1.00 | 54.33 |
| ATOM | 19807 | C | GLU | 2950 | 44.971 | 33.340 | 5.128 | 1.00 | 53.26 |
| ATOM | 19808 | O | GLU | 2950 | 46.168 | 33.404 | 5.413 | 1.00 | 53.12 |
| ATOM | 19809 | N | VAL | 2951 | 44.272 | 32.213 | 5.210 | 1.00 | 53.26 |
| ATOM | 19810 | CA | VAL | 2951 | 44.903 | 30.971 | 5.641 | 1.00 | 53.29 |
| ATOM | 19811 | CB | VAL | 2951 | 43.849 | 29.891 | 5.970 | 1.00 | 53.31 |
| ATOM | 19812 | CG1 | VAL | 2951 | 44.533 | 28.561 | 6.259 | 1.00 | 53.04 |
| ATOM | 19813 | CG2 | VAL | 2951 | 43.025 | 30.327 | 7.174 | 1.00 | 52.46 |
| ATOM | 19814 | C | VAL | 2951 | 45.849 | 30.442 | 4.568 | 1.00 | 53.26 |
| ATOM | 19815 | O | VAL | 2951 | 47.012 | 30.145 | 4.847 | 1.00 | 53.25 |
| ATOM | 19816 | N | GLU | 2952 | 45.348 | 30.328 | 3.342 | 1.00 | 53.64 |
| ATOM | 19817 | CA | GLU | 2952 | 46.157 | 29.835 | 2.232 | 1.00 | 54.22 |
| ATOM | 19818 | CB | GLU | 2952 | 45.328 | 29.793 | 0.947 | 1.00 | 54.72 |
| ATOM | 19819 | CG | GLU | 2952 | 46.082 | 29.252 | −0.261 | 1.00 | 55.51 |
| ATOM | 19820 | CD | GLU | 2952 | 45.257 | 29.300 | −1.532 | 1.00 | 56.73 |
| ATOM | 19821 | OE1 | GLU | 2952 | 45.774 | 28.890 | −2.594 | 1.00 | 57.69 |
| ATOM | 19822 | OE2 | GLU | 2952 | 44.092 | 29.750 | −1.473 | 1.00 | 56.56 |
| ATOM | 19823 | C | GLU | 2952 | 47.370 | 30.730 | 2.023 | 1.00 | 54.22 |
| ATOM | 19824 | O | GLU | 2952 | 48.430 | 30.266 | 1.608 | 1.00 | 54.53 |
| ATOM | 19825 | N | SER | 2953 | 47.202 | 32.015 | 2.318 | 1.00 | 54.17 |
| ATOM | 19826 | CA | SER | 2953 | 48.272 | 32.993 | 2.162 | 1.00 | 54.43 |
| ATOM | 19827 | CB | SER | 2953 | 47.690 | 34.407 | 2.129 | 1.00 | 54.29 |
| ATOM | 19828 | OG | SER | 2953 | 46.736 | 34.542 | 1.092 | 1.00 | 56.12 |
| ATOM | 19829 | C | SER | 2953 | 49.279 | 32.892 | 3.297 | 1.00 | 54.04 |
| ATOM | 19830 | O | SER | 2953 | 50.487 | 32.943 | 3.074 | 1.00 | 54.27 |
| ATOM | 19831 | N | GLY | 2954 | 48.771 | 32.750 | 4.516 | 1.00 | 53.77 |
| ATOM | 19832 | CA | GLY | 2954 | 49.642 | 32.657 | 5.672 | 1.00 | 53.06 |
| ATOM | 19833 | C | GLY | 2954 | 49.442 | 33.832 | 6.609 | 1.00 | 52.88 |
| ATOM | 19834 | O | GLY | 2954 | 49.975 | 33.854 | 7.719 | 1.00 | 51.86 |
| ATOM | 19835 | N | VAL | 2955 | 48.670 | 34.815 | 6.156 | 1.00 | 52.98 |
| ATOM | 19836 | CA | VAL | 2955 | 48.388 | 36.003 | 6.953 | 1.00 | 53.65 |
| ATOM | 19837 | CB | VAL | 2955 | 47.426 | 36.952 | 6.214 | 1.00 | 54.01 |
| ATOM | 19838 | CG1 | VAL | 2955 | 47.201 | 38.208 | 7.039 | 1.00 | 53.59 |
| ATOM | 19839 | CG2 | VAL | 2955 | 47.990 | 37.300 | 4.846 | 1.00 | 54.14 |
| ATOM | 19840 | C | VAL | 2955 | 47.756 | 35.606 | 8.281 | 1.00 | 54.39 |
| ATOM | 19841 | O | VAL | 2955 | 48.003 | 36.236 | 9.309 | 1.00 | 53.92 |
| ATOM | 19842 | N | TYR | 2956 | 46.934 | 34.560 | 8.249 | 1.00 | 54.97 |
| ATOM | 19843 | CA | TYR | 2956 | 46.271 | 34.070 | 9.451 | 1.00 | 55.91 |
| ATOM | 19844 | CB | TYR | 2956 | 44.756 | 34.258 | 9.346 | 1.00 | 56.40 |
| ATOM | 19845 | CG | TYR | 2956 | 44.014 | 33.837 | 10.595 | 1.00 | 57.63 |
| ATOM | 19846 | CD1 | TYR | 2956 | 44.082 | 34.600 | 11.761 | 1.00 | 57.68 |
| ATOM | 19847 | CE1 | TYR | 2956 | 43.423 | 34.202 | 12.923 | 1.00 | 58.33 |
| ATOM | 19848 | CD2 | TYR | 2956 | 43.265 | 32.660 | 10.621 | 1.00 | 58.32 |
| ATOM | 19849 | CE2 | TYR | 2956 | 42.603 | 32.251 | 11.778 | 1.00 | 58.41 |
| ATOM | 19850 | CZ | TYR | 2956 | 42.686 | 33.027 | 12.924 | 1.00 | 58.65 |
| ATOM | 19851 | OH | TYR | 2956 | 42.033 | 32.631 | 14.069 | 1.00 | 58.77 |
| ATOM | 19852 | C | TYR | 2956 | 46.577 | 32.591 | 9.675 | 1.00 | 56.09 |
| ATOM | 19853 | O | TYR | 2956 | 46.478 | 31.779 | 8.754 | 1.00 | 56.17 |
| ATOM | 19854 | N | PRO | 2957 | 46.962 | 32.223 | 10.908 | 1.00 | 56.16 |
| ATOM | 19855 | CD | PRO | 2957 | 47.177 | 30.824 | 11.320 | 1.00 | 56.15 |
| ATOM | 19856 | CA | PRO | 2957 | 47.109 | 33.126 | 12.052 | 1.00 | 56.59 |
| ATOM | 19857 | CB | PRO | 2957 | 47.081 | 32.173 | 13.239 | 1.00 | 56.39 |
| ATOM | 19858 | CG | PRO | 2957 | 47.791 | 30.980 | 12.698 | 1.00 | 56.47 |
| ATOM | 19859 | C | PRO | 2957 | 48.405 | 33.928 | 11.984 | 1.00 | 57.30 |
| ATOM | 19860 | O | PRO | 2957 | 49.413 | 33.452 | 11.462 | 1.00 | 57.47 |
| ATOM | 19861 | N | GLY | 2958 | 48.370 | 35.148 | 12.512 | 1.00 | 58.29 |
| ATOM | 19862 | CA | GLY | 2958 | 49.551 | 35.991 | 12.504 | 1.00 | 59.19 |
| ATOM | 19863 | C | GLY | 2958 | 50.469 | 35.674 | 13.668 | 1.00 | 59.90 |
| ATOM | 19864 | O | GLY | 2958 | 50.359 | 34.612 | 14.282 | 1.00 | 59.93 |
| ATOM | 19865 | N | GLU | 2959 | 51.376 | 36.595 | 13.975 | 1.00 | 60.38 |
| ATOM | 19866 | CA | GLU | 2959 | 52.312 | 36.400 | 15.076 | 1.00 | 60.87 |
| ATOM | 19867 | CB | GLU | 2959 | 53.457 | 37.413 | 14.988 | 1.00 | 61.40 |
| ATOM | 19868 | CG | GLU | 2959 | 54.521 | 37.227 | 16.060 | 1.00 | 61.82 |
| ATOM | 19869 | CD | GLU | 2959 | 55.285 | 35.926 | 15.903 | 1.00 | 62.12 |
| ATOM | 19870 | OE1 | GLU | 2959 | 56.067 | 35.579 | 16.813 | 1.00 | 62.78 |
| ATOM | 19871 | OE2 | GLU | 2959 | 55.108 | 35.251 | 14.867 | 1.00 | 62.30 |
| ATOM | 19872 | C | GLU | 2959 | 51.605 | 36.548 | 16.419 | 1.00 | 60.84 |
| ATOM | 19873 | O | GLU | 2959 | 51.933 | 35.855 | 17.384 | 1.00 | 60.59 |
| ATOM | 19874 | N | GLU | 2960 | 50.634 | 37.454 | 16.477 | 1.00 | 61.19 |
| ATOM | 19875 | CA | GLU | 2960 | 49.889 | 37.689 | 17.709 | 1.00 | 61.99 |
| ATOM | 19876 | CB | GLU | 2960 | 48.941 | 38.882 | 17.554 | 1.00 | 62.57 |
| ATOM | 19877 | CG | GLU | 2960 | 49.390 | 39.924 | 16.546 | 1.00 | 63.87 |
| ATOM | 19878 | CD | GLU | 2960 | 49.017 | 39.551 | 15.123 | 1.00 | 64.41 |
| ATOM | 19879 | OE1 | GLU | 2960 | 47.804 | 39.475 | 14.828 | 1.00 | 64.20 |
| ATOM | 19880 | OE2 | GLU | 2960 | 49.933 | 39.330 | 14.301 | 1.00 | 64.88 |
| ATOM | 19881 | C | GLU | 2960 | 49.077 | 36.450 | 18.052 | 1.00 | 61.91 |
| ATOM | 19882 | O | GLU | 2960 | 48.947 | 36.081 | 19.219 | 1.00 | 62.32 |
| ATOM | 19883 | N | HIS | 2961 | 48.534 | 35.813 | 17.020 | 1.00 | 61.34 |
| ATOM | 19884 | CA | HIS | 2961 | 47.723 | 34.615 | 17.196 | 1.00 | 60.73 |
| ATOM | 19885 | CB | HIS | 2961 | 46.855 | 34.386 | 15.957 | 1.00 | 59.89 |
| ATOM | 19886 | CG | HIS | 2961 | 46.116 | 35.607 | 15.504 | 1.00 | 58.97 |
| ATOM | 19887 | CD2 | HIS | 2961 | 46.080 | 36.231 | 14.302 | 1.00 | 58.22 |
| ATOM | 19888 | ND1 | HIS | 2961 | 45.285 | 36.326 | 16.334 | 1.00 | 58.84 |
| ATOM | 19889 | CE1 | HIS | 2961 | 44.768 | 37.341 | 15.665 | 1.00 | 58.19 |
| ATOM | 19890 | NE2 | HIS | 2961 | 45.233 | 37.306 | 14.430 | 1.00 | 57.99 |
| ATOM | 19891 | C | HIS | 2961 | 48.599 | 33.391 | 17.437 | 1.00 | 60.75 |
| ATOM | 19892 | O | HIS | 2961 | 48.106 | 32.261 | 17.467 | 1.00 | 60.65 |
| ATOM | 19893 | N | SER | 2962 | 49.897 | 33.619 | 17.614 | 1.00 | 60.45 |
| ATOM | 19894 | CA | SER | 2962 | 50.839 | 32.529 | 17.844 | 1.00 | 60.48 |
| ATOM | 19895 | CB | SER | 2962 | 52.007 | 32.630 | 16.862 | 1.00 | 60.35 |
| ATOM | 19896 | OG | SER | 2962 | 51.552 | 32.553 | 15.522 | 1.00 | 59.02 |
| ATOM | 19897 | C | SER | 2962 | 51.370 | 32.518 | 19.273 | 1.00 | 60.73 |
| ATOM | 19898 | O | SER | 2962 | 51.339 | 33.534 | 19.964 | 1.00 | 60.10 |
| ATOM | 19899 | N | PHE | 2963 | 51.851 | 31.356 | 19.708 | 1.00 | 61.57 |
| ATOM | 19900 | CA | PHE | 2963 | 52.394 | 31.194 | 21.053 | 1.00 | 62.25 |
| ATOM | 19901 | CB | PHE | 2963 | 51.644 | 30.091 | 21.807 | 1.00 | 63.03 |
| ATOM | 19902 | CG | PHE | 2963 | 50.248 | 30.470 | 22.214 | 1.00 | 64.44 |
| ATOM | 19903 | CD1 | PHE | 2963 | 49.278 | 30.750 | 21.256 | 1.00 | 64.83 |
| ATOM | 19904 | CD2 | PHE | 2963 | 49.903 | 30.552 | 23.560 | 1.00 | 64.71 |
| ATOM | 19905 | CE1 | PHE | 2963 | 47.983 | 31.107 | 21.632 | 1.00 | 64.99 |
| ATOM | 19906 | CE2 | PHE | 2963 | 48.614 | 30.906 | 23.948 | 1.00 | 65.11 |
| ATOM | 19907 | CZ | PHE | 2963 | 47.651 | 31.185 | 22.981 | 1.00 | 65.30 |
| ATOM | 19908 | C | PHE | 2963 | 53.880 | 30.853 | 21.006 | 1.00 | 62.31 |
| ATOM | 19909 | O | PHE | 2963 | 54.379 | 30.323 | 20.011 | 1.00 | 61.84 |
| ATOM | 19910 | N | HIS | 2964 | 54.577 | 31.156 | 22.096 | 1.00 | 62.35 |
| ATOM | 19911 | CA | HIS | 2964 | 56.007 | 30.897 | 22.201 | 1.00 | 62.42 |
| ATOM | 19912 | CB | HIS | 2964 | 56.795 | 32.135 | 21.772 | 1.00 | 62.17 |
| ATOM | 19913 | CG | HIS | 2964 | 56.654 | 32.468 | 20.318 | 1.00 | 62.21 |
| ATOM | 19914 | CD2 | HIS | 2964 | 56.082 | 33.526 | 19.696 | 1.00 | 62.06 |
| ATOM | 19915 | ND1 | HIS | 2964 | 57.137 | 31.652 | 19.319 | 1.00 | 62.16 |
| ATOM | 19916 | CE1 | HIS | 2964 | 56.870 | 32.193 | 18.143 | 1.00 | 62.55 |
| ATOM | 19917 | NE2 | HIS | 2964 | 56.230 | 33.330 | 18.344 | 1.00 | 62.23 |
| ATOM | 19918 | C | HIS | 2964 | 56.382 | 30.514 | 23.628 | 1.00 | 62.87 |
| ATOM | 19919 | O | HIS | 2964 | 57.101 | 29.507 | 23.800 | 1.00 | 63.17 |
| ATOM | 19920 | OXT | HIS | 2964 | 55.958 | 31.232 | 24.558 | 1.00 | 63.88 |
| ATOM | 19921 | C1 | KPL | 2965 | 38.359 | 24.260 | 19.395 | 1.00 | 44.32 |
| ATOM | 19922 | C2 | KPL | 2965 | 38.509 | 22.957 | 18.586 | 1.00 | 44.37 |
| ATOM | 19923 | C3 | KPL | 2965 | 38.070 | 23.219 | 17.138 | 1.00 | 44.11 |
| ATOM | 19924 | C4 | KPL | 2965 | 39.994 | 22.528 | 18.576 | 1.00 | 44.41 |
| ATOM | 19925 | O1 | KPL | 2965 | 40.466 | 22.273 | 19.906 | 1.00 | 46.59 |
| ATOM | 19926 | C5 | KPL | 2965 | 37.616 | 21.846 | 19.201 | 1.00 | 44.46 |
| ATOM | 19927 | O2 | KPL | 2965 | 38.120 | 20.811 | 19.596 | 1.00 | 45.29 |
| ATOM | 19928 | C6 | KPL | 2965 | 36.112 | 22.005 | 19.331 | 1.00 | 44.39 |
| ATOM | 19929 | O3 | KPL | 2965 | 35.550 | 23.016 | 18.951 | 1.00 | 44.59 |
| ATOM | 19930 | O4 | KPL | 2965 | 35.382 | 21.012 | 19.874 | 1.00 | 43.64 |
| ATOM | 19931 | MG+2 | MG2 | 3001 | 3.994 | 24.216 | 47.085 | 1.00 | 41.37 |
| ATOM | 19932 | MG+2 | MG2 | 3002 | 6.567 | 28.508 | 15.105 | 1.00 | 41.66 |
| ATOM | 19933 | MG+2 | MG2 | 3003 | −3.352 | 1.040 | 0.322 | 1.00 | 26.64 |
| ATOM | 19934 | MG+2 | MG2 | 3004 | −12.375 | −19.811 | 23.437 | 1.00 | 24.57 |
| ATOM | 19935 | MG+2 | MG2 | 3005 | −7.605 | −5.894 | 52.220 | 1.00 | 34.50 |
| ATOM | 19936 | MG+2 | MG2 | 3006 | 27.460 | −4.705 | 0.894 | 1.00 | 30.26 |
| ATOM | 19937 | MG+2 | MG2 | 3007 | 17.331 | −30.253 | 18.884 | 1.00 | 32.29 |
| ATOM | 19938 | MG+2 | MG2 | 3008 | 19.663 | −21.554 | 49.976 | 1.00 | 25.48 |
| ATOM | 19939 | MG+2 | MG2 | 3009 | 31.302 | 8.730 | 51.343 | 1.00 | 41.34 |
| ATOM | 19940 | MG+2 | MG2 | 3010 | 36.277 | 19.579 | 21.091 | 1.00 | 52.60 |
| ATOM | 19941 | OH2 | WAT | 3011 | 31.424 | −16.107 | 39.470 | 1.00 | 10.76 |
| ATOM | 19942 | OH2 | WAT | 3012 | 12.698 | −18.611 | 32.844 | 1.00 | 10.49 |
| ATOM | 19943 | OH2 | WAT | 3013 | −8.246 | 1.949 | 11.544 | 1.00 | 13.19 |
| ATOM | 19944 | OH2 | WAT | 3014 | 27.207 | −31.545 | 22.513 | 1.00 | 13.62 |
| ATOM | 19945 | OH2 | WAT | 3015 | 25.517 | −21.182 | 20.257 | 1.00 | 10.14 |
| ATOM | 19946 | OH2 | WAT | 3016 | −7.674 | 17.525 | 37.246 | 1.00 | 13.74 |
| ATOM | 19947 | OH2 | WAT | 3017 | −2.159 | −6.488 | 21.338 | 1.00 | 15.76 |
| ATOM | 19948 | OH2 | WAT | 3018 | 0.835 | −9.327 | 15.447 | 1.00 | 11.52 |
| ATOM | 19949 | OH2 | WAT | 3019 | 3.764 | −32.000 | 22.104 | 1.00 | 11.84 |
| ATOM | 19950 | OH2 | WAT | 3020 | 37.123 | −1.512 | 39.521 | 1.00 | 16.58 |
| ATOM | 19951 | OH2 | WAT | 3021 | 17.933 | −46.120 | 14.538 | 1.00 | 11.66 |
| ATOM | 19952 | OH2 | WAT | 3022 | 10.775 | −17.843 | 17.172 | 1.00 | 15.20 |
| ATOM | 19953 | OH2 | WAT | 3023 | 15.784 | −11.897 | 13.470 | 1.00 | 11.48 |
| ATOM | 19954 | OH2 | WAT | 3024 | −14.336 | 22.570 | −15.394 | 1.00 | 13.53 |
| ATOM | 19955 | OH2 | WAT | 3025 | 12.577 | −9.564 | 9.580 | 1.00 | 14.31 |
| ATOM | 19956 | OH2 | WAT | 3026 | −23.367 | 12.451 | 2.123 | 1.00 | 20.69 |
| ATOM | 19957 | OH2 | WAT | 3027 | 0.767 | −19.320 | 30.486 | 1.00 | 16.05 |
| ATOM | 19958 | OH2 | WAT | 3028 | −0.982 | −8.203 | 33.657 | 1.00 | 15.14 |
| ATOM | 19959 | OH2 | WAT | 3029 | 5.689 | 5.614 | 45.308 | 1.00 | 11.24 |
| ATOM | 19960 | OE2 | WAT | 3030 | 4.143 | 4.088 | 43.662 | 1.00 | 15.33 |
| ATOM | 19961 | OH2 | WAT | 3031 | −16.843 | −2.595 | 47.938 | 1.00 | 11.00 |
| ATOM | 19962 | OH2 | WAT | 3032 | 12.241 | 13.450 | 38.158 | 1.00 | 23.70 |
| ATOM | 19963 | OH2 | WAT | 3033 | −5.960 | −12.449 | 28.625 | 1.00 | 14.48 |
| ATOM | 19964 | OH2 | WAT | 3034 | 16.382 | −12.732 | 16.031 | 1.00 | 14.41 |
| ATOM | 19965 | OH2 | WAT | 3035 | 33.192 | −17.361 | 37.635 | 1.00 | 14.11 |
| ATOM | 19966 | OH2 | WAT | 3036 | 29.576 | −3.425 | 53.736 | 1.00 | 16.58 |
| ATOM | 19967 | OH2 | WAT | 3037 | 25.016 | −14.473 | 37.183 | 1.00 | 18.91 |
| ATOM | 19968 | OH2 | WAT | 3038 | 10.453 | 7.434 | 47.450 | 1.00 | 13.74 |
| ATOM | 19969 | OH2 | WAT | 3039 | 0.953 | −32.769 | 9.383 | 1.00 | 15.20 |
| ATOM | 19970 | OH2 | WAT | 3040 | −7.328 | 8.718 | −18.137 | 1.00 | 18.80 |
| ATOM | 19971 | OH2 | WAT | 3041 | −8.835 | 2.730 | 43.501 | 1.00 | 10.87 |
| ATOM | 19972 | OH2 | WAT | 3042 | −1.580 | −10.794 | 33.592 | 1.00 | 12.62 |
| ATOM | 19973 | OH2 | WAT | 3043 | −12.914 | −8.541 | 27.617 | 1.00 | 12.73 |
| ATOM | 19974 | OH2 | WAT | 3044 | 21.710 | 13.409 | 25.403 | 1.00 | 31.57 |
| ATOM | 19975 | OH2 | WAT | 3045 | 7.683 | −20.302 | 30.445 | 1.00 | 10.85 |
| ATOM | 19976 | OH2 | WAT | 3046 | 1.102 | −30.671 | 18.260 | 1.00 | 15.68 |
| ATOM | 19977 | OH2 | WAT | 3047 | 28.173 | 7.798 | 31.821 | 1.00 | 20.07 |
| ATOM | 19978 | OH2 | WAT | 3048 | −1.179 | −14.949 | 31.883 | 1.00 | 13.42 |
| ATOM | 19979 | OH2 | WAT | 3049 | 13.221 | 14.116 | 19.297 | 1.00 | 20.69 |
| ATOM | 19980 | OH2 | WAT | 3050 | −2.044 | 20.189 | 37.466 | 1.00 | 16.68 |
| ATOM | 19981 | OH2 | WAT | 3051 | 17.876 | −14.432 | 39.307 | 1.00 | 16.30 |
| ATOM | 19982 | OH2 | WAT | 3052 | −0.744 | −30.549 | 20.671 | 1.00 | 17.77 |
| ATOM | 19983 | OH2 | WAT | 3053 | 35.481 | −39.258 | 29.142 | 1.00 | 12.21 |
| ATOM | 19984 | OH2 | WAT | 3054 | −9.507 | 4.070 | −12.790 | 1.00 | 16.79 |
| ATOM | 19985 | OH2 | WAT | 3055 | 31.604 | −1.451 | 46.191 | 1.00 | 14.27 |
| ATOM | 19986 | OH2 | WAT | 3056 | −14.406 | 5.407 | 38.561 | 1.00 | 16.53 |
| ATOM | 19987 | OH2 | WAT | 3057 | −0.928 | −11.952 | 36.038 | 1.00 | 12.23 |
| ATOM | 19988 | OH2 | WAT | 3058 | 0.468 | −17.148 | 54.501 | 1.00 | 17.45 |
| ATOM | 19989 | OH2 | WAT | 3059 | 5.125 | −36.602 | 35.570 | 1.00 | 20.20 |
| ATOM | 19990 | OH2 | WAT | 3060 | 0.620 | −51.357 | 23.066 | 1.00 | 14.88 |
| ATOM | 19991 | OH2 | WAT | 3061 | 17.818 | −20.565 | 43.493 | 1.00 | 14.36 |
| ATOM | 19992 | OH2 | WAT | 3062 | 23.320 | 7.615 | 14.657 | 1.00 | 12.79 |
| ATOM | 19993 | OH2 | WAT | 3063 | −13.311 | 2.901 | 39.033 | 1.00 | 16.11 |
| ATOM | 19994 | OH2 | WAT | 3064 | 32.528 | −20.167 | 23.589 | 1.00 | 13.88 |
| ATOM | 19995 | OH2 | WAT | 3065 | 13.191 | −4.951 | 48.336 | 1.00 | 19.12 |
| ATOM | 19996 | OH2 | WAT | 3066 | −11.736 | 7.247 | 3.147 | 1.00 | 13.94 |
| ATOM | 19997 | OH2 | WAT | 3067 | 32.586 | −0.717 | 43.888 | 1.00 | 14.86 |
| ATOM | 19998 | OH2 | WAT | 3068 | 11.327 | 19.214 | 30.461 | 1.00 | 15.85 |
| ATOM | 19999 | OH2 | WAT | 3069 | 19.105 | −45.037 | 17.671 | 1.00 | 11.69 |
| ATOM | 20000 | OH2 | WAT | 3070 | −9.957 | −7.454 | 6.721 | 1.00 | 17.25 |
| ATOM | 20001 | OH2 | WAT | 3071 | 9.085 | −16.151 | 57.247 | 1.00 | 14.59 |
| ATOM | 20002 | OH2 | WAT | 3072 | 39.426 | 3.322 | 24.588 | 1.00 | 19.14 |
| ATOM | 20003 | OH2 | WAT | 3073 | 3.530 | 9.016 | 39.444 | 1.00 | 15.75 |
| ATOM | 20004 | OH2 | WAT | 3074 | −10.708 | 15.943 | −14.582 | 1.00 | 14.94 |
| ATOM | 20005 | OH2 | WAT | 3075 | −0.027 | −17.054 | 29.261 | 1.00 | 12.27 |
| ATOM | 20006 | OH2 | WAT | 3076 | 14.927 | −5.859 | −4.822 | 1.00 | 16.81 |
| ATOM | 20007 | OH2 | WAT | 3077 | −6.247 | 19.997 | 21.778 | 1.00 | 16.82 |
| ATOM | 20008 | OH2 | WAT | 3078 | 23.194 | −3.748 | 41.708 | 1.00 | 13.63 |
| ATOM | 20009 | OH2 | WAT | 3079 | 3.044 | −14.551 | 15.690 | 1.00 | 11.83 |
| ATOM | 20010 | OH2 | WAT | 3080 | 15.856 | −44.437 | 15.495 | 1.00 | 16.34 |
| ATOM | 20011 | OH2 | WAT | 3081 | 2.658 | −6.744 | 11.642 | 1.00 | 13.95 |
| ATOM | 20012 | OH2 | WAT | 3082 | 18.097 | −3.425 | 45.902 | 1.00 | 14.60 |
| ATOM | 20013 | OH2 | WAT | 3083 | 17.808 | −10.614 | 17.279 | 1.00 | 20.73 |
| ATOM | 20014 | OH2 | WAT | 3084 | 17.717 | −23.621 | 16.919 | 1.00 | 14.50 |
| ATOM | 20015 | OH2 | WAT | 3085 | 20.450 | −28.027 | 39.405 | 1.00 | 20.21 |
| ATOM | 20016 | OH2 | WAT | 3086 | −16.370 | 8.639 | 40.975 | 1.00 | 19.28 |
| ATOM | 20017 | OH2 | WAT | 3087 | 25.222 | −0.313 | 11.241 | 1.00 | 15.89 |
| ATOM | 20018 | OH2 | WAT | 3088 | 11.033 | −3.025 | −5.467 | 1.00 | 16.78 |
| ATOM | 20019 | OH2 | WAT | 3089 | −2.490 | −3.684 | 17.486 | 1.00 | 14.21 |
| ATOM | 20020 | OH2 | WAT | 3090 | 22.173 | −49.823 | 28.580 | 1.00 | 17.88 |
| ATOM | 20021 | OH2 | WAT | 3091 | −8.999 | −16.320 | 27.976 | 1.00 | 15.29 |
| ATOM | 20022 | OH2 | WAT | 3092 | −19.387 | −2.818 | 23.319 | 1.00 | 14.53 |
| ATOM | 20023 | OH2 | WAT | 3093 | 35.100 | −8.958 | 15.354 | 1.00 | 16.00 |
| ATOM | 20024 | OH2 | WAT | 3094 | −10.894 | 6.982 | 14.192 | 1.00 | 14.83 |
| ATOM | 20025 | OH2 | WAT | 3095 | 38.364 | −11.130 | 14.395 | 1.00 | 14.78 |
| ATOM | 20026 | OH2 | WAT | 3096 | −14.355 | 9.093 | 12.720 | 1.00 | 16.02 |
| ATOM | 20027 | OH2 | WAT | 3097 | −2.640 | −2.142 | 6.218 | 1.00 | 14.25 |
| ATOM | 20028 | OH2 | WAT | 3098 | 14.236 | −16.887 | 34.025 | 1.00 | 14.37 |
| ATOM | 20029 | OH2 | WAT | 3099 | −6.286 | −20.729 | 35.017 | 1.00 | 17.92 |
| ATOM | 20030 | OH2 | WAT | 3100 | 5.618 | −54.564 | 19.599 | 1.00 | 18.87 |
| ATOM | 20031 | OH2 | WAT | 3101 | 24.798 | 12.998 | 34.734 | 1.00 | 20.61 |
| ATOM | 20032 | OH2 | WAT | 3102 | 18.157 | −48.389 | 22.944 | 1.00 | 18.26 |
| ATOM | 20033 | OH2 | WAT | 3103 | 3.448 | −22.348 | 28.656 | 1.00 | 16.83 |
| ATOM | 20034 | OH2 | WAT | 3104 | 37.310 | 16.574 | 60.707 | 1.00 | 13.63 |
| ATOM | 20035 | OH2 | WAT | 3105 | 7.399 | 10.930 | 14.456 | 1.00 | 15.32 |
| ATOM | 20036 | OH2 | WAT | 3106 | −4.065 | −6.934 | 32.716 | 1.00 | 13.85 |
| ATOM | 20037 | OH2 | WAT | 3107 | 26.681 | −0.650 | 6.361 | 1.00 | 16.84 |
| ATOM | 20038 | OH2 | WAT | 3108 | 2.621 | −29.273 | 20.371 | 1.00 | 18.02 |
| ATOM | 20039 | OH2 | WAT | 3109 | −8.257 | 9.695 | −15.506 | 1.00 | 17.18 |
| ATOM | 20040 | OH2 | WAT | 3110 | −5.408 | 3.177 | −13.054 | 1.00 | 16.87 |
| ATOM | 20041 | OH2 | WAT | 3111 | −14.226 | −15.880 | 35.035 | 1.00 | 19.49 |
| ATOM | 20042 | OH2 | WAT | 3112 | 8.844 | −18.039 | 14.940 | 1.00 | 13.32 |
| ATOM | 20043 | OH2 | WAT | 3113 | 10.416 | −45.629 | 8.580 | 1.00 | 19.28 |
| ATOM | 20044 | OH2 | WAT | 3114 | 24.321 | −21.344 | 39.443 | 1.00 | 14.51 |
| ATOM | 20045 | OH2 | WAT | 3115 | 32.620 | −1.644 | 10.966 | 1.00 | 18.64 |
| ATOM | 20046 | OH2 | WAT | 3116 | 21.077 | −7.973 | 16.352 | 1.00 | 16.09 |
| ATOM | 20047 | OH2 | WAT | 3117 | 11.416 | 8.982 | 43.352 | 1.00 | 19.27 |
| ATOM | 20048 | OH2 | WAT | 3118 | 35.781 | −10.327 | 4.606 | 1.00 | 17.92 |
| ATOM | 20049 | OH2 | WAT | 3119 | 11.474 | 7.062 | 8.572 | 1.00 | 18.56 |
| ATOM | 20050 | OH2 | WAT | 3120 | 17.899 | −18.896 | 18.746 | 1.00 | 14.76 |
| ATOM | 20051 | OH2 | WAT | 3121 | 9.407 | 1.445 | −5.210 | 1.00 | 17.30 |
| ATOM | 20052 | OH2 | WAT | 3122 | 11.378 | −25.783 | 40.687 | 1.00 | 19.55 |
| ATOM | 20053 | OH2 | WAT | 3123 | 30.129 | −21.017 | 24.522 | 1.00 | 15.05 |
| ATOM | 20054 | OH2 | WAT | 3124 | 15.046 | −43.858 | 18.014 | 1.00 | 11.77 |
| ATOM | 20055 | OH2 | WAT | 3125 | −9.121 | 2.705 | 47.977 | 1.00 | 17.81 |
| ATOM | 20056 | OH2 | WAT | 3126 | 34.332 | −14.474 | 60.657 | 1.00 | 24.94 |
| ATOM | 20057 | OH2 | WAT | 3127 | −3.766 | 0.667 | 55.823 | 1.00 | 21.51 |
| ATOM | 20058 | OH2 | WAT | 3128 | 33.157 | −10.971 | 35.543 | 1.00 | 17.96 |
| ATOM | 20059 | OH2 | WAT | 3129 | 20.042 | −48.084 | 29.393 | 1.00 | 17.39 |
| ATOM | 20060 | OH2 | WAT | 3130 | 7.739 | −10.910 | −9.034 | 1.00 | 16.41 |
| ATOM | 20061 | OH2 | WAT | 3131 | −2.901 | −28.128 | 17.100 | 1.00 | 11.64 |
| ATOM | 20062 | OH2 | WAT | 3132 | 8.775 | −17.442 | 26.955 | 1.00 | 15.53 |
| ATOM | 20063 | OH2 | WAT | 3133 | 10.635 | −14.815 | 5.424 | 1.00 | 17.26 |
| ATOM | 20064 | OH2 | WAT | 3134 | −1.407 | 15.539 | 35.202 | 1.00 | 21.03 |
| ATOM | 20065 | OH2 | WAT | 3135 | 22.262 | −3.590 | 54.476 | 1.00 | 17.12 |
| ATOM | 20066 | OH2 | WAT | 3136 | −13.401 | −21.508 | 25.158 | 1.00 | 27.63 |
| ATOM | 20067 | OH2 | WAT | 3137 | 5.128 | −2.847 | 47.443 | 1.00 | 12.45 |
| ATOM | 20068 | OH2 | WAT | 3138 | −15.619 | −3.485 | 24.661 | 1.00 | 9.97 |
| ATOM | 20069 | OH2 | WAT | 3139 | −2.531 | −40.976 | 12.328 | 1.00 | 13.52 |
| ATOM | 20070 | OH2 | WAT | 3140 | −1.909 | −4.892 | 36.246 | 1.00 | 16.91 |
| ATOM | 20071 | OH2 | WAT | 3141 | −19.604 | 14.972 | 6.233 | 1.00 | 20.13 |
| ATOM | 20072 | OH2 | WAT | 3142 | −6.017 | −42.085 | 21.509 | 1.00 | 16.39 |
| ATOM | 20073 | OH2 | WAT | 3143 | −7.945 | 16.282 | −13.491 | 1.00 | 17.83 |
| ATOM | 20074 | OH2 | WAT | 3144 | 30.702 | 28.827 | 44.434 | 1.00 | 22.57 |
| ATOM | 20075 | OH2 | WAT | 3145 | −9.294 | 18.400 | 35.274 | 1.00 | 15.61 |
| ATOM | 20076 | OH2 | WAT | 3146 | 30.373 | −21.453 | 48.978 | 1.00 | 13.66 |
| ATOM | 20077 | OH2 | WAT | 3147 | −11.335 | 18.904 | 38.629 | 1.00 | 18.20 |
| ATOM | 20078 | OH2 | WAT | 3148 | 5.189 | 16.665 | 30.863 | 1.00 | 16.04 |
| ATOM | 20079 | OH2 | WAT | 3149 | 8.219 | −17.924 | 40.764 | 1.00 | 16.89 |
| ATOM | 20080 | OH2 | WAT | 3150 | 41.387 | −1.649 | 40.145 | 1.00 | 18.07 |
| ATOM | 20081 | OH2 | WAT | 3151 | −3.245 | −39.921 | 9.833 | 1.00 | 18.23 |
| ATOM | 20082 | OH2 | WAT | 3152 | 8.198 | 9.770 | 11.939 | 1.00 | 15.65 |
| ATOM | 20083 | OH2 | WAT | 3153 | −13.314 | 6.554 | 15.618 | 1.00 | 21.95 |
| ATOM | 20084 | OH2 | WAT | 3154 | 11.910 | −17.540 | 37.668 | 1.00 | 13.80 |
| ATOM | 20085 | OH2 | WAT | 3155 | −20.114 | −12.582 | 21.243 | 1.00 | 16.42 |
| ATOM | 20086 | OH2 | WAT | 3156 | −1.281 | −11.060 | 5.278 | 1.00 | 17.26 |
| ATOM | 20087 | OH2 | WAT | 3157 | −17.431 | 3.089 | 39.729 | 1.00 | 12.26 |
| ATOM | 20088 | OH2 | WAT | 3158 | −3.548 | −35.710 | 27.746 | 1.00 | 16.68 |
| ATOM | 20089 | OH2 | WAT | 3159 | −17.120 | 5.018 | 37.848 | 1.00 | 16.94 |
| ATOM | 20090 | OH2 | WAT | 3160 | 13.477 | −2.627 | 54.843 | 1.00 | 18.64 |
| ATOM | 20091 | OH2 | WAT | 3161 | −5.243 | −43.014 | 23.849 | 1.00 | 19.60 |
| ATOM | 20092 | OH2 | WAT | 3162 | −13.199 | −10.151 | 29.903 | 1.00 | 15.06 |
| ATOM | 20093 | OH2 | WAT | 3163 | 2.054 | −26.977 | 54.316 | 1.00 | 21.37 |
| ATOM | 20094 | OH2 | WAT | 3164 | 23.993 | −29.599 | 61.802 | 1.00 | 20.85 |
| ATOM | 20095 | OH2 | WAT | 3165 | 24.933 | 1.982 | 44.938 | 1.00 | 17.95 |
| ATOM | 20096 | OH2 | WAT | 3166 | 16.321 | −50.335 | 21.944 | 1.00 | 14.95 |
| ATOM | 20097 | OH2 | WAT | 3167 | 4.185 | −10.137 | 40.452 | 1.00 | 18.49 |
| ATOM | 20098 | OH2 | WAT | 3168 | −6.542 | 2.530 | 38.971 | 1.00 | 18.82 |
| ATOM | 20099 | OH2 | WAT | 3169 | 13.980 | −5.493 | 11.385 | 1.00 | 15.38 |
| ATOM | 20100 | OH2 | WAT | 3170 | 23.236 | 4.920 | 15.059 | 1.00 | 16.06 |
| ATOM | 20101 | OH2 | WAT | 3171 | −18.040 | 24.374 | 52.309 | 1.00 | 20.78 |
| ATOM | 20102 | OH2 | WAT | 3172 | 37.479 | 14.626 | 63.159 | 1.00 | 18.93 |
| ATOM | 20103 | OH2 | WAT | 3173 | 40.548 | 4.987 | 47.799 | 1.00 | 20.00 |
| ATOM | 20104 | OH2 | WAT | 3174 | −7.860 | 3.921 | 45.943 | 1.00 | 16.64 |
| ATOM | 20105 | OH2 | WAT | 3175 | 4.737 | −16.653 | 43.656 | 1.00 | 23.24 |
| ATOM | 20106 | OH2 | WAT | 3176 | 19.671 | −2.177 | 44.183 | 1.00 | 15.91 |
| ATOM | 20107 | OH2 | WAT | 3177 | 9.864 | −0.921 | 9.302 | 1.00 | 19.67 |
| ATOM | 20108 | OH2 | WAT | 3178 | 25.798 | −18.855 | 39.813 | 1.00 | 15.04 |
| ATOM | 20109 | OH2 | WAT | 3179 | −3.409 | −1.361 | 49.300 | 1.00 | 15.09 |
| ATOM | 20110 | OH2 | WAT | 3180 | 5.816 | 22.238 | 40.960 | 1.00 | 19.83 |
| ATOM | 20111 | OH2 | WAT | 3181 | 21.304 | 15.686 | 38.364 | 1.00 | 19.23 |
| ATOM | 20112 | OH2 | WAT | 3182 | 33.264 | −12.303 | 53.961 | 1.00 | 22.43 |
| ATOM | 20113 | OH2 | WAT | 3183 | −19.721 | −30.018 | 18.220 | 1.00 | 17.02 |
| ATOM | 20114 | OH2 | WAT | 3184 | 30.616 | 19.768 | 70.046 | 1.00 | 18.96 |
| ATOM | 20115 | OH2 | WAT | 3185 | 13.397 | 11.894 | 3.881 | 1.00 | 22.84 |
| ATOM | 20116 | OH2 | WAT | 3186 | 21.307 | 4.413 | 11.054 | 1.00 | 18.87 |
| ATOM | 20117 | OH2 | WAT | 3187 | −14.018 | 16.237 | −13.520 | 1.00 | 13.75 |
| ATOM | 20118 | OH2 | WAT | 3188 | 27.233 | 3.845 | 48.990 | 1.00 | 15.76 |
| ATOM | 20119 | OH2 | WAT | 3189 | 18.322 | −35.199 | 37.473 | 1.00 | 21.41 |
| ATOM | 20120 | OH2 | WAT | 3190 | 3.346 | 10.973 | 16.658 | 1.00 | 17.09 |
| ATOM | 20121 | OH2 | WAT | 3191 | −1.268 | 0.118 | 45.115 | 1.00 | 20.12 |
| ATOM | 20122 | OH2 | WAT | 3192 | 8.866 | −56.007 | 22.359 | 1.00 | 22.50 |
| ATOM | 20123 | OH2 | WAT | 3193 | 4.755 | 11.704 | −21.103 | 1.00 | 21.21 |
| ATOM | 20124 | OH2 | WAT | 3194 | 29.496 | 7.747 | 5.130 | 1.00 | 24.25 |
| ATOM | 20125 | OH2 | WAT | 3195 | 6.916 | −2.587 | 12.344 | 1.00 | 16.98 |
| ATOM | 20126 | OH2 | WAT | 3196 | 6.422 | 15.829 | 26.439 | 1.00 | 19.29 |
| ATOM | 20127 | OH2 | WAT | 3197 | −13.894 | −11.050 | 64.371 | 1.00 | 18.77 |
| ATOM | 20128 | OH2 | WAT | 3198 | 3.453 | −13.631 | 20.195 | 1.00 | 16.50 |
| ATOM | 20129 | OH2 | WAT | 3199 | 13.511 | −27.216 | 36.881 | 1.00 | 18.17 |
| ATOM | 20130 | OH2 | WAT | 3200 | 10.479 | −16.462 | 40.801 | 1.00 | 14.13 |
| ATOM | 20131 | OH2 | WAT | 3201 | 19.084 | 1.615 | 46.456 | 1.00 | 19.77 |
| ATOM | 20132 | OH2 | WAT | 3202 | 13.651 | −21.751 | 8.937 | 1.00 | 19.12 |
| ATOM | 20133 | OH2 | WAT | 3203 | 30.258 | −1.193 | 39.421 | 1.00 | 23.25 |
| ATOM | 20134 | OH2 | WAT | 3204 | 22.221 | −46.203 | 12.674 | 1.00 | 18.08 |
| ATOM | 20135 | OH2 | WAT | 3205 | −10.806 | 20.587 | 22.314 | 1.00 | 22.59 |
| ATOM | 20136 | OH2 | WAT | 3206 | 25.446 | 8.006 | 4.362 | 1.00 | 22.71 |
| ATOM | 20137 | OH2 | WAT | 3207 | −1.437 | −43.569 | 11.596 | 1.00 | 14.68 |
| ATOM | 20138 | OH2 | WAT | 3208 | 6.165 | 17.099 | 16.294 | 1.00 | 19.35 |
| ATOM | 20139 | OH2 | WAT | 3209 | 0.456 | −5.728 | 18.027 | 1.00 | 16.11 |
| ATOM | 20140 | OH2 | WAT | 3210 | 17.554 | −15.937 | 28.233 | 1.00 | 15.55 |
| ATOM | 20141 | OH2 | WAT | 3211 | 2.388 | −14.201 | 39.957 | 1.00 | 16.30 |
| ATOM | 20142 | OH2 | WAT | 3212 | −8.445 | −43.392 | 21.151 | 1.00 | 15.83 |
| ATOM | 20143 | OH2 | WAT | 3213 | −1.149 | −2.138 | 11.071 | 1.00 | 16.20 |
| ATOM | 20144 | OH2 | WAT | 3214 | 16.006 | −18.966 | 9.491 | 1.00 | 22.11 |
| ATOM | 20145 | OH2 | WAT | 3215 | 40.917 | 14.465 | 61.223 | 1.00 | 17.97 |
| ATOM | 20146 | OH2 | WAT | 3216 | 20.418 | −7.448 | 40.411 | 1.00 | 18.22 |
| ATOM | 20147 | OH2 | WAT | 3217 | 7.210 | 6.558 | −23.993 | 1.00 | 22.59 |
| ATOM | 20148 | OH2 | WAT | 3218 | 13.270 | −1.290 | −5.622 | 1.00 | 17.55 |
| ATOM | 20149 | OH2 | WAT | 3219 | 16.193 | −6.804 | 65.086 | 1.00 | 23.17 |
| ATOM | 20150 | OH2 | WAT | 3220 | 1.345 | 6.829 | 53.690 | 1.00 | 19.22 |
| ATOM | 20151 | OH2 | WAT | 3221 | −24.487 | −28.861 | 12.302 | 1.00 | 24.58 |
| ATOM | 20152 | OH2 | WAT | 3222 | −5.861 | 6.465 | 53.062 | 1.00 | 22.85 |
| ATOM | 20153 | OH2 | WAT | 3223 | 2.196 | −17.385 | 9.566 | 1.00 | 14.82 |
| ATOM | 20154 | OH2 | WAT | 3224 | 5.907 | 18.688 | 37.560 | 1.00 | 23.71 |
| ATOM | 20155 | OH2 | WAT | 3225 | 9.404 | −6.985 | −17.364 | 1.00 | 18.51 |
| ATOM | 20156 | OH2 | WAT | 3226 | −19.508 | 18.345 | −4.134 | 1.00 | 18.87 |
| ATOM | 20157 | OH2 | WAT | 3227 | 43.927 | 19.766 | 59.453 | 1.00 | 22.41 |
| ATOM | 20158 | OH2 | WAT | 3228 | 10.197 | 3.381 | 10.668 | 1.00 | 16.51 |
| ATOM | 20159 | OH2 | WAT | 3229 | −14.378 | 18.764 | 53.775 | 1.00 | 20.79 |
| ATOM | 20160 | OH2 | WAT | 3230 | −20.438 | −16.179 | 61.040 | 1.00 | 22.89 |
| ATOM | 20161 | OH2 | WAT | 3231 | 30.282 | −36.634 | 32.910 | 1.00 | 15.84 |
| ATOM | 20162 | OH2 | WAT | 3232 | 39.788 | 13.875 | 64.578 | 1.00 | 17.48 |
| ATOM | 20163 | OH2 | WAT | 3233 | −21.813 | −14.345 | 7.149 | 1.00 | 19.96 |
| ATOM | 20164 | OH2 | WAT | 3234 | 3.481 | −16.263 | 23.755 | 1.00 | 24.36 |
| ATOM | 20165 | OH2 | WAT | 3235 | 32.740 | 7.544 | 50.333 | 1.00 | 19.77 |
| ATOM | 20166 | OH2 | WAT | 3236 | −16.527 | −40.590 | 22.042 | 1.00 | 15.97 |
| ATOM | 20167 | OH2 | WAT | 3237 | −20.321 | −1.940 | 29.261 | 1.00 | 17.79 |
| ATOM | 20168 | OH2 | WAT | 3238 | 6.910 | −16.348 | 54.593 | 1.00 | 23.74 |
| ATOM | 20169 | OH2 | WAT | 3239 | 32.211 | −4.106 | 12.342 | 1.00 | 12.74 |
| ATOM | 20170 | OH2 | WAT | 3240 | 6.833 | −38.308 | 33.861 | 1.00 | 27.01 |
| ATOM | 20171 | OH2 | WAT | 3241 | −5.435 | 3.091 | 15.587 | 1.00 | 25.68 |
| ATOM | 20172 | OH2 | WAT | 3242 | 5.162 | −5.435 | 9.145 | 1.00 | 15.66 |
| ATOM | 20173 | OH2 | WAT | 3243 | −12.976 | 18.875 | 1.055 | 1.00 | 26.56 |
| ATOM | 20174 | OH2 | WAT | 3244 | 4.941 | −50.548 | 27.523 | 1.00 | 18.25 |
| ATOM | 20175 | OH2 | WAT | 3245 | 23.502 | 4.496 | 17.712 | 1.00 | 21.76 |
| ATOM | 20176 | OH2 | WAT | 3246 | 29.781 | −8.409 | −12.106 | 1.00 | 22.34 |
| ATOM | 20177 | OH2 | WAT | 3247 | 16.179 | 20.627 | 27.934 | 1.00 | 17.27 |
| ATOM | 20178 | OH2 | WAT | 3248 | 25.630 | 6.081 | 35.873 | 1.00 | 24.47 |
| ATOM | 20179 | OH2 | WAT | 3249 | 19.075 | 2.823 | 8.867 | 1.00 | 17.88 |
| ATOM | 20180 | OH2 | WAT | 3250 | 24.463 | −1.978 | 55.349 | 1.00 | 19.50 |
| ATOM | 20181 | OH2 | WAT | 3251 | 6.006 | −7.737 | 8.134 | 1.00 | 18.83 |
| ATOM | 20182 | OH2 | WAT | 3252 | 24.297 | −22.682 | 41.765 | 1.00 | 22.36 |
| ATOM | 20183 | OH2 | WAT | 3253 | −14.877 | 8.810 | 15.278 | 1.00 | 19.04 |
| ATOM | 20184 | OH2 | WAT | 3254 | 6.808 | −2.819 | 49.732 | 1.00 | 18.14 |
| ATOM | 20185 | OH2 | WAT | 3255 | 26.166 | 5.603 | 20.868 | 1.00 | 21.17 |
| ATOM | 20186 | OH2 | WAT | 3256 | 4.305 | −15.545 | 56.992 | 1.00 | 16.97 |
| ATOM | 20187 | OH2 | WAT | 3257 | 13.421 | −15.839 | 15.404 | 1.00 | 19.34 |
| ATOM | 20188 | OH2 | WAT | 3258 | −12.696 | −19.398 | 26.900 | 1.00 | 26.15 |
| ATOM | 20189 | OH2 | WAT | 3259 | 5.047 | −18.352 | 55.566 | 1.00 | 20.95 |
| ATOM | 20190 | OH2 | WAT | 3260 | −21.140 | −30.966 | 20.114 | 1.00 | 22.15 |
| ATOM | 20191 | OH2 | WAT | 3261 | −0.778 | 32.093 | 65.599 | 1.00 | 22.89 |
| ATOM | 20192 | OH2 | WAT | 3262 | −4.568 | −12.190 | 8.019 | 1.00 | 19.08 |
| ATOM | 20193 | OH2 | WAT | 3263 | −9.797 | 1.121 | −14.744 | 1.00 | 15.93 |
| ATOM | 20194 | OH2 | WAT | 3264 | −6.689 | 23.825 | 46.121 | 1.00 | 15.64 |
| ATOM | 20195 | OH2 | WAT | 3265 | 14.061 | 12.604 | 42.176 | 1.00 | 31.75 |
| ATOM | 20196 | OH2 | WAT | 3266 | −0.527 | −0.897 | 42.399 | 1.00 | 20.09 |
| ATOM | 20197 | OH2 | WAT | 3267 | −0.194 | 33.276 | 58.037 | 1.00 | 23.96 |
| ATOM | 20198 | OH2 | WAT | 3268 | 24.901 | −17.073 | 22.925 | 1.00 | 20.92 |
| ATOM | 20199 | OH2 | WAT | 3269 | 41.330 | −8.601 | 15.013 | 1.00 | 23.33 |
| ATOM | 20200 | OH2 | WAT | 3270 | 4.620 | −8.727 | −6.543 | 1.00 | 24.77 |
| ATOM | 20201 | OH2 | WAT | 3271 | 35.246 | −17.451 | 41.068 | 1.00 | 18.93 |
| ATOM | 20202 | OH2 | WAT | 3272 | 25.285 | 11.482 | 32.566 | 1.00 | 19.14 |
| ATOM | 20203 | OH2 | WAT | 3273 | 41.753 | −19.624 | 48.472 | 1.00 | 23.10 |
| ATOM | 20204 | OH2 | WAT | 3274 | 4.615 | 0.859 | 46.531 | 1.00 | 16.54 |
| ATOM | 20205 | OH2 | WAT | 3275 | 31.542 | −15.777 | 28.303 | 1.00 | 20.38 |
| ATOM | 20206 | OH2 | WAT | 3276 | 19.527 | 11.849 | 39.254 | 1.00 | 15.70 |
| ATOM | 20207 | OH2 | WAT | 3277 | 33.147 | −23.760 | 25.248 | 1.00 | 16.58 |
| ATOM | 20208 | OH2 | WAT | 3278 | −5.916 | −49.095 | 13.770 | 1.00 | 29.58 |
| ATOM | 20209 | OH2 | WAT | 3279 | 23.794 | −15.430 | 33.966 | 1.00 | 21.14 |
| ATOM | 20210 | OH2 | WAT | 3280 | −14.122 | 0.241 | −14.020 | 1.00 | 21.60 |
| ATOM | 20211 | OH2 | WAT | 3281 | −31.126 | −7.465 | 48.968 | 1.00 | 18.37 |
| ATOM | 20212 | OH2 | WAT | 3282 | 33.182 | 4.867 | 26.769 | 1.00 | 29.67 |
| ATOM | 20213 | OH2 | WAT | 3283 | 24.781 | 8.897 | 32.613 | 1.00 | 23.60 |
| ATOM | 20214 | OH2 | WAT | 3284 | 37.324 | −32.782 | 27.936 | 1.00 | 20.55 |
| ATOM | 20215 | OH2 | WAT | 3285 | 36.703 | −2.932 | 24.810 | 1.00 | 20.31 |
| ATOM | 20216 | OH2 | WAT | 3286 | 16.737 | 6.043 | −10.203 | 1.00 | 14.77 |
| ATOM | 20217 | OH2 | WAT | 3287 | 42.281 | 10.193 | 65.227 | 1.00 | 20.86 |
| ATOM | 20218 | OH2 | WAT | 3288 | −24.643 | −24.852 | 6.883 | 1.00 | 22.27 |
| ATOM | 20219 | OH2 | WAT | 3289 | 3.427 | 33.777 | 58.006 | 1.00 | 17.87 |
| ATOM | 20220 | OH2 | WAT | 3290 | 26.392 | 2.408 | 17.399 | 1.00 | 19.04 |
| ATOM | 20221 | OH2 | WAT | 3291 | −18.654 | −5.950 | 66.016 | 1.00 | 23.24 |
| ATOM | 20222 | OH2 | WAT | 3292 | −13.093 | −15.210 | 22.791 | 1.00 | 20.23 |
| ATOM | 20223 | OH2 | WAT | 3293 | 9.173 | −6.311 | 51.088 | 1.00 | 24.64 |
| ATOM | 20224 | OH2 | WAT | 3294 | 38.564 | −37.037 | 23.715 | 1.00 | 17.22 |
| ATOM | 20225 | OH2 | WAT | 3295 | −23.611 | −28.491 | 18.445 | 1.00 | 21.94 |
| ATOM | 20226 | OH2 | WAT | 3296 | 43.474 | −20.767 | 2.623 | 1.00 | 21.92 |
| ATOM | 20227 | OH2 | WAT | 3297 | 5.370 | −10.917 | 64.991 | 1.00 | 20.49 |
| ATOM | 20228 | OH2 | WAT | 3298 | 21.104 | 12.701 | 14.311 | 1.00 | 20.74 |
| ATOM | 20229 | OH2 | WAT | 3299 | −8.121 | 27.589 | 65.671 | 1.00 | 30.46 |
| ATOM | 20230 | OH2 | WAT | 3300 | 17.972 | 5.158 | 7.455 | 1.00 | 16.91 |
| ATOM | 20231 | OH2 | WAT | 3301 | 10.494 | −0.984 | −3.411 | 1.00 | 21.18 |
| ATOM | 20232 | OH2 | WAT | 3302 | 4.032 | 42.243 | 10.424 | 1.00 | 30.91 |
| ATOM | 20233 | OH2 | WAT | 3303 | 24.441 | −27.550 | 68.904 | 1.00 | 18.25 |
| ATOM | 20234 | OH2 | WAT | 3304 | 6.219 | 21.783 | 14.079 | 1.00 | 25.97 |
| ATOM | 20235 | OH2 | WAT | 3305 | 23.593 | −1.831 | 13.125 | 1.00 | 19.74 |
| ATOM | 20236 | OH2 | WAT | 3306 | −17.736 | 1.565 | 54.302 | 1.00 | 19.29 |
| ATOM | 20237 | OH2 | WAT | 3307 | 13.850 | −2.899 | −3.293 | 1.00 | 22.15 |
| ATOM | 20238 | OH2 | WAT | 3308 | 34.332 | −43.289 | 29.457 | 1.00 | 20.15 |
| ATOM | 20239 | OH2 | WAT | 3309 | −5.197 | −6.814 | 1.773 | 1.00 | 19.55 |
| ATOM | 20240 | OH2 | WAT | 3310 | −12.090 | 18.072 | 36.228 | 1.00 | 19.58 |
| ATOM | 20241 | OH2 | WAT | 3311 | 17.354 | 10.254 | 43.120 | 1.00 | 34.77 |
| ATOM | 20242 | OH2 | WAT | 3312 | −3.313 | 11.757 | 19.670 | 1.00 | 26.41 |
| ATOM | 20243 | OH2 | WAT | 3313 | 8.528 | −13.751 | 41.287 | 1.00 | 21.05 |
| ATOM | 20244 | OH2 | WAT | 3314 | 32.008 | −20.113 | 6.850 | 1.00 | 25.55 |
| ATOM | 20245 | OH2 | WAT | 3315 | 13.281 | 16.160 | 15.117 | 1.00 | 24.88 |
| ATOM | 20246 | OH2 | WAT | 3316 | 28.691 | 9.379 | 7.117 | 1.00 | 20.59 |
| ATOM | 20247 | OH2 | WAT | 3317 | 23.789 | −6.461 | 61.180 | 1.00 | 26.68 |
| ATOM | 20248 | OH2 | WAT | 3318 | 1.515 | 36.096 | 58.072 | 1.00 | 18.91 |
| ATOM | 20249 | OH2 | WAT | 3319 | 48.872 | 11.459 | 42.531 | 1.00 | 27.53 |
| ATOM | 20250 | OH2 | WAT | 3320 | 5.225 | −25.223 | 34.596 | 1.00 | 14.92 |
| ATOM | 20251 | OH2 | WAT | 3321 | 37.106 | −22.432 | 3.847 | 1.00 | 17.63 |
| ATOM | 20252 | OH2 | WAT | 3322 | −9.345 | −4.853 | 27.183 | 1.00 | 24.67 |
| ATOM | 20253 | OH2 | WAT | 3323 | 34.721 | −21.213 | 36.495 | 1.00 | 21.39 |
| ATOM | 20254 | OH2 | WAT | 3324 | 32.652 | −14.927 | −13.607 | 1.00 | 25.32 |
| ATOM | 20255 | OH2 | WAT | 3325 | −13.339 | 21.021 | −8.922 | 1.00 | 22.80 |
| ATOM | 20256 | OH2 | WAT | 3326 | −3.296 | −1.036 | 16.162 | 1.00 | 24.45 |
| ATOM | 20257 | OH2 | WAT | 3327 | 9.522 | −32.401 | 16.459 | 1.00 | 23.66 |
| ATOM | 20258 | OH2 | WAT | 3328 | 18.151 | 15.055 | 63.606 | 1.00 | 19.05 |
| ATOM | 20259 | OH2 | WAT | 3329 | 6.249 | −47.125 | 10.644 | 1.00 | 21.08 |
| ATOM | 20260 | OH2 | WAT | 3330 | −9.192 | 11.917 | 33.840 | 1.00 | 20.79 |
| ATOM | 20261 | OH2 | WAT | 3331 | 4.631 | −19.143 | 15.726 | 1.00 | 20.59 |
| ATOM | 20262 | OH2 | WAT | 3332 | 18.911 | 18.491 | 41.568 | 1.00 | 24.07 |
| ATOM | 20263 | OH2 | WAT | 3333 | −17.813 | 22.262 | 45.797 | 1.00 | 26.58 |
| ATOM | 20264 | OH2 | WAT | 3334 | 10.713 | −28.271 | 37.324 | 1.00 | 25.49 |
| ATOM | 20265 | OH2 | WAT | 3335 | 29.350 | −5.186 | 2.361 | 1.00 | 31.20 |
| ATOM | 20266 | OH2 | WAT | 3336 | 7.196 | −5.221 | 46.352 | 1.00 | 22.56 |
| ATOM | 20267 | OH2 | WAT | 3337 | 54.614 | 10.503 | 48.370 | 1.00 | 21.87 |
| ATOM | 20268 | OH2 | WAT | 3338 | 12.377 | −24.993 | 37.751 | 1.00 | 21.71 |
| ATOM | 20269 | OH2 | WAT | 3339 | 16.863 | 5.321 | 48.931 | 1.00 | 23.14 |
| ATOM | 20270 | OH2 | WAT | 3340 | −8.724 | 19.145 | 20.888 | 1.00 | 19.11 |
| ATOM | 20271 | OH2 | WAT | 3341 | 13.381 | −19.714 | −4.203 | 1.00 | 16.96 |
| ATOM | 20272 | OH2 | WAT | 3342 | 11.141 | 15.500 | −6.398 | 1.00 | 25.52 |
| ATOM | 20273 | OH2 | WAT | 3343 | −0.771 | 12.885 | 18.816 | 1.00 | 31.48 |
| ATOM | 20274 | OH2 | WAT | 3344 | −16.965 | 4.474 | 14.900 | 1.00 | 22.48 |
| ATOM | 20275 | OH2 | WAT | 3345 | 19.464 | 21.926 | 68.546 | 1.00 | 24.61 |
| ATOM | 20276 | OH2 | WAT | 3346 | −8.665 | −0.567 | 10.532 | 1.00 | 17.50 |
| ATOM | 20277 | OH2 | WAT | 3347 | 19.548 | −3.441 | 41.810 | 1.00 | 18.14 |
| ATOM | 20278 | OH2 | WAT | 3348 | 3.984 | 5.093 | 41.179 | 1.00 | 19.65 |
| ATOM | 20279 | OH2 | WAT | 3349 | 34.101 | 4.713 | 6.355 | 1.00 | 26.04 |
| ATOM | 20280 | OH2 | WAT | 3350 | 9.510 | −54.905 | 26.309 | 1.00 | 22.92 |
| ATOM | 20281 | OH2 | WAT | 3351 | 2.738 | 2.375 | 54.788 | 1.00 | 21.38 |
| ATOM | 20282 | OH2 | WAT | 3352 | 38.200 | −3.877 | 39.235 | 1.00 | 17.67 |
| ATOM | 20283 | OH2 | WAT | 3353 | 10.728 | 14.126 | 13.818 | 1.00 | 21.22 |
| ATOM | 20284 | OH2 | WAT | 3354 | −1.810 | −3.501 | −10.761 | 1.00 | 32.21 |
| ATOM | 20285 | OH2 | WAT | 3355 | 23.131 | 20.084 | 52.484 | 1.00 | 21.25 |
| ATOM | 20286 | OH2 | WAT | 3356 | 13.275 | 37.450 | 61.583 | 1.00 | 23.72 |
| ATOM | 20287 | OH2 | WAT | 3357 | 14.902 | −27.854 | 39.228 | 1.00 | 17.88 |
| ATOM | 20288 | OH2 | WAT | 3358 | 17.196 | 0.708 | −19.731 | 1.00 | 22.56 |
| ATOM | 20289 | OH2 | WAT | 3359 | 12.894 | 27.941 | 72.812 | 1.00 | 22.10 |
| ATOM | 20290 | OH2 | WAT | 3360 | 51.803 | 18.337 | 55.429 | 1.00 | 23.28 |
| ATOM | 20291 | OH2 | WAT | 3361 | 23.951 | 22.297 | 38.321 | 1.00 | 19.39 |
| ATOM | 20292 | OH2 | WAT | 3362 | 28.231 | 10.502 | 25.782 | 1.00 | 29.33 |
| ATOM | 20293 | OH2 | WAT | 3363 | −19.632 | −10.100 | 40.923 | 1.00 | 24.00 |
| ATOM | 20294 | OH2 | WAT | 3364 | 0.381 | −18.881 | 70.964 | 1.00 | 24.24 |
| ATOM | 20295 | OH2 | WAT | 3365 | 41.050 | −6.012 | 15.791 | 1.00 | 25.68 |
| ATOM | 20296 | OH2 | WAT | 3366 | 25.036 | −21.624 | 17.420 | 1.00 | 19.61 |
| ATOM | 20297 | OH2 | WAT | 3367 | 20.757 | −12.469 | 18.162 | 1.00 | 19.66 |
| ATOM | 20298 | OH2 | WAT | 3368 | 25.031 | −39.595 | 34.156 | 1.00 | 17.92 |
| ATOM | 20299 | OH2 | WAT | 3369 | −29.570 | −5.102 | 46.308 | 1.00 | 22.63 |
| ATOM | 20300 | OH2 | WAT | 3370 | −4.963 | 11.491 | 58.938 | 1.00 | 26.60 |
| ATOM | 20301 | OH2 | WAT | 3371 | −1.293 | 15.137 | −16.952 | 1.00 | 22.14 |
| ATOM | 20302 | OH2 | WAT | 3372 | 7.371 | 10.814 | 64.052 | 1.00 | 22.29 |
| ATOM | 20303 | OH2 | WAT | 3373 | 16.271 | −27.044 | 44.698 | 1.00 | 21.59 |
| ATOM | 20304 | OH2 | WAT | 3374 | −9.825 | −19.028 | 36.378 | 1.00 | 15.87 |
| ATOM | 20305 | OH2 | WAT | 3375 | −24.137 | −9.482 | 10.270 | 1.00 | 23.02 |
| ATOM | 20306 | OH2 | WAT | 3376 | −13.705 | −13.121 | 62.268 | 1.00 | 20.24 |
| ATOM | 20307 | OH2 | WAT | 3377 | −11.384 | 0.851 | 7.106 | 1.00 | 24.50 |
| ATOM | 20308 | OH2 | WAT | 3378 | 41.978 | −12.471 | 14.199 | 1.00 | 23.08 |
| ATOM | 20309 | OH2 | WAT | 3379 | −3.147 | −14.517 | 6.808 | 1.00 | 23.32 |
| ATOM | 20310 | OH2 | WAT | 3380 | −0.728 | −27.767 | 19.798 | 1.00 | 21.29 |
| ATOM | 20311 | OH2 | WAT | 3381 | −0.354 | −4.894 | −20.116 | 1.00 | 24.42 |
| ATOM | 20312 | OH2 | WAT | 3382 | −21.061 | −11.267 | 68.198 | 1.00 | 30.13 |
| ATOM | 20313 | OH2 | WAT | 3383 | −14.706 | −12.293 | 29.188 | 1.00 | 20.50 |
| ATOM | 20314 | OH2 | WAT | 3384 | 43.391 | 2.510 | 23.335 | 1.00 | 25.76 |
| ATOM | 20315 | OH2 | WAT | 3385 | 3.515 | 39.540 | 67.856 | 1.00 | 26.52 |
| ATOM | 20316 | OH2 | WAT | 3386 | −1.928 | 8.812 | −25.826 | 1.00 | 33.73 |
| ATOM | 20317 | OH2 | WAT | 3387 | 50.385 | 2.215 | 42.463 | 1.00 | 29.09 |
| ATOM | 20318 | OH2 | WAT | 3388 | 17.087 | 16.878 | 20.716 | 1.00 | 37.71 |
| ATOM | 20319 | OH2 | WAT | 3389 | 8.298 | 15.973 | 7.123 | 1.00 | 32.77 |
| ATOM | 20320 | OH2 | WAT | 3390 | 34.661 | −21.425 | 25.109 | 1.00 | 19.60 |
| ATOM | 20321 | OH2 | WAT | 3391 | 19.966 | −51.330 | 15.176 | 1.00 | 26.16 |
| ATOM | 20322 | OH2 | WAT | 3392 | −9.059 | −4.483 | 51.040 | 1.00 | 20.52 |
| ATOM | 20323 | OH2 | WAT | 3393 | 25.707 | −32.629 | 33.317 | 1.00 | 17.81 |
| ATOM | 20324 | OH2 | WAT | 3394 | 5.534 | 19.085 | 5.472 | 1.00 | 33.14 |
| ATOM | 20325 | OH2 | WAT | 3395 | 14.622 | 19.551 | 55.214 | 1.00 | 19.96 |
| ATOM | 20326 | OH2 | WAT | 3396 | 24.959 | −14.087 | 20.835 | 1.00 | 28.72 |
| ATOM | 20327 | OH2 | WAT | 3397 | 13.290 | 17.903 | 38.782 | 1.00 | 20.42 |
| ATOM | 20328 | OH2 | WAT | 3398 | −5.862 | −4.263 | 56.954 | 1.00 | 32.48 |
| ATOM | 20329 | OH2 | WAT | 3399 | 6.562 | 15.994 | 19.058 | 1.00 | 25.19 |
| ATOM | 20330 | OH2 | WAT | 3400 | 14.970 | 3.846 | −18.083 | 1.00 | 19.32 |
| ATOM | 20331 | OH2 | WAT | 3401 | −7.124 | 15.004 | −19.783 | 1.00 | 24.92 |
| ATOM | 20332 | OH2 | WAT | 3402 | 34.119 | −8.981 | −11.775 | 1.00 | 21.64 |
| ATOM | 20333 | OH2 | WAT | 3403 | 5.848 | 15.691 | 37.447 | 1.00 | 21.96 |
| ATOM | 20334 | OH2 | WAT | 3404 | −4.384 | 5.307 | 38.927 | 1.00 | 28.93 |
| ATOM | 20335 | OH2 | WAT | 3405 | −8.462 | 19.489 | −3.041 | 1.00 | 23.36 |
| ATOM | 20336 | OH2 | WAT | 3406 | 21.504 | −14.699 | 19.950 | 1.00 | 29.34 |
| ATOM | 20337 | OH2 | WAT | 3407 | −21.088 | 4.227 | 39.634 | 1.00 | 22.61 |
| ATOM | 20338 | OH2 | WAT | 3408 | −5.619 | −10.651 | 5.927 | 1.00 | 24.23 |
| ATOM | 20339 | OH2 | WAT | 3409 | 52.693 | 1.167 | 48.206 | 1.00 | 21.20 |
| ATOM | 20340 | OH2 | WAT | 3410 | −16.221 | −9.667 | 65.559 | 1.00 | 20.52 |
| ATOM | 20341 | OH2 | WAT | 3411 | −25.467 | −9.423 | 43.328 | 1.00 | 24.48 |
| ATOM | 20342 | OH2 | WAT | 3412 | −20.988 | 4.741 | 56.477 | 1.00 | 26.96 |
| ATOM | 20343 | OH2 | WAT | 3413 | 19.198 | 18.956 | 55.185 | 1.00 | 23.07 |
| ATOM | 20344 | OH2 | WAT | 3414 | −24.253 | −31.934 | 12.026 | 1.00 | 26.24 |
| ATOM | 20345 | OH2 | WAT | 3415 | 12.072 | 18.774 | 35.664 | 1.00 | 23.52 |
| ATOM | 20346 | OH2 | WAT | 3416 | −12.454 | 3.743 | 32.390 | 1.00 | 17.67 |
| ATOM | 20347 | OH2 | WAT | 3417 | −18.447 | 6.868 | 40.520 | 1.00 | 24.96 |
| ATOM | 20348 | OH2 | WAT | 3418 | 6.103 | −8.025 | 44.128 | 1.00 | 27.53 |
| ATOM | 20349 | OH2 | WAT | 3419 | −15.120 | −36.912 | 4.961 | 1.00 | 24.66 |
| ATOM | 20350 | OH2 | WAT | 3420 | 18.742 | 5.029 | 46.950 | 1.00 | 28.56 |
| ATOM | 20351 | OH2 | WAT | 3421 | 14.049 | −14.727 | 75.131 | 1.00 | 22.18 |
| ATOM | 20352 | OH2 | WAT | 3422 | 25.532 | −35.284 | 59.886 | 1.00 | 25.87 |
| ATOM | 20353 | OH2 | WAT | 3423 | 14.242 | 4.864 | 4.801 | 1.00 | 26.11 |
| ATOM | 20354 | OH2 | WAT | 3424 | 2.505 | −8.983 | 62.076 | 1.00 | 23.05 |
| ATOM | 20355 | OH2 | WAT | 3425 | 6.236 | 16.265 | 5.305 | 1.00 | 24.87 |
| ATOM | 20356 | OH2 | WAT | 3426 | 7.628 | −26.541 | 40.424 | 1.00 | 27.19 |
| ATOM | 20357 | OH2 | WAT | 3427 | 10.084 | 5.888 | 54.362 | 1.00 | 18.70 |
| ATOM | 20358 | OH2 | WAT | 3428 | −27.669 | −4.824 | 17.585 | 1.00 | 22.63 |
| ATOM | 20359 | OH2 | WAT | 3429 | 16.131 | −57.526 | 20.651 | 1.00 | 27.80 |
| ATOM | 20360 | OH2 | WAT | 3430 | 33.810 | −10.761 | 21.863 | 1.00 | 20.66 |
| ATOM | 20361 | OH2 | WAT | 3431 | 38.906 | −5.089 | −12.711 | 1.00 | 32.66 |
| ATOM | 20362 | OH2 | WAT | 3432 | 3.036 | 6.220 | 15.329 | 1.00 | 21.16 |
| ATOM | 20363 | OH2 | WAT | 3433 | −5.338 | 0.253 | 17.916 | 1.00 | 28.50 |
| ATOM | 20364 | OH2 | WAT | 3434 | −6.431 | 25.378 | 65.275 | 1.00 | 28.77 |
| ATOM | 20365 | OH2 | WAT | 3435 | 30.183 | −20.851 | 68.133 | 1.00 | 27.16 |
| ATOM | 20366 | OH2 | WAT | 3436 | 43.659 | −22.333 | −1.106 | 1.00 | 24.34 |
| ATOM | 20367 | OH2 | WAT | 3437 | 30.093 | 19.087 | 34.416 | 1.00 | 35.14 |
| ATOM | 20368 | OH2 | WAT | 3438 | −30.035 | −9.535 | 13.285 | 1.00 | 30.59 |
| ATOM | 20369 | OH2 | WAT | 3439 | −17.340 | −10.617 | 62.281 | 1.00 | 21.35 |
| ATOM | 20370 | OH2 | WAT | 3440 | 4.191 | −8.621 | 64.549 | 1.00 | 25.99 |
| ATOM | 20371 | OH2 | WAT | 3441 | 26.000 | −4.755 | 17.793 | 1.00 | 27.01 |
| ATOM | 20372 | OH2 | WAT | 3442 | −7.412 | 21.856 | −20.059 | 1.00 | 21.29 |
| ATOM | 20373 | OH2 | WAT | 3443 | −7.879 | 17.002 | 4.254 | 1.00 | 26.02 |
| ATOM | 20374 | OH2 | WAT | 3444 | −9.087 | −0.369 | 8.012 | 1.00 | 22.06 |
| ATOM | 20375 | OH2 | WAT | 3445 | −23.952 | 1.102 | −9.034 | 1.00 | 28.67 |
| ATOM | 20376 | OH2 | WAT | 3446 | −10.640 | 18.228 | 58.792 | 1.00 | 24.48 |
| ATOM | 20377 | OH2 | WAT | 3447 | −28.943 | 1.338 | 47.872 | 1.00 | 24.52 |
| ATOM | 20378 | OH2 | WAT | 3448 | 6.713 | −26.895 | 47.186 | 1.00 | 25.17 |
| ATOM | 20379 | OH2 | WAT | 3449 | −19.226 | 4.149 | 7.394 | 1.00 | 20.22 |
| ATOM | 20380 | OH2 | WAT | 3450 | 16.180 | −54.471 | 16.613 | 1.00 | 19.97 |
| ATOM | 20381 | OH2 | WAT | 3451 | 7.659 | −6.722 | −19.699 | 1.00 | 24.66 |
| ATOM | 20382 | OH2 | WAT | 3452 | 37.335 | −8.150 | 16.691 | 1.00 | 18.64 |
| ATOM | 20383 | OH2 | WAT | 3453 | 20.745 | 12.571 | 18.819 | 1.00 | 24.11 |
| ATOM | 20384 | OH2 | WAT | 3454 | −17.203 | −1.956 | 26.377 | 1.00 | 19.51 |
| ATOM | 20385 | OH2 | WAT | 3455 | 15.288 | −15.336 | 31.913 | 1.00 | 18.81 |
| ATOM | 20386 | OH2 | WAT | 3456 | 8.707 | −48.754 | 10.866 | 1.00 | 27.33 |
| ATOM | 20387 | OH2 | WAT | 3457 | 15.343 | 19.454 | 38.824 | 1.00 | 23.12 |
| ATOM | 20388 | OH2 | WAT | 3458 | 20.577 | −15.545 | 35.392 | 1.00 | 27.66 |
| ATOM | 20389 | OH2 | WAT | 3459 | 41.003 | 1.947 | 26.461 | 1.00 | 21.44 |
| ATOM | 20390 | OH2 | WAT | 3460 | −19.312 | 17.129 | −0.087 | 1.00 | 24.24 |
| ATOM | 20391 | OH2 | WAT | 3461 | 41.814 | −22.072 | 59.142 | 1.00 | 29.16 |
| ATOM | 20392 | OH2 | WAT | 3462 | −23.822 | −2.922 | 29.368 | 1.00 | 26.46 |
| ATOM | 20393 | OH2 | WAT | 3463 | −19.777 | −17.397 | 63.713 | 1.00 | 22.86 |
| ATOM | 20394 | OH2 | WAT | 3464 | 9.958 | 49.419 | 21.653 | 1.00 | 31.91 |
| ATOM | 20395 | OH2 | WAT | 3465 | −14.397 | 0.840 | 6.887 | 1.00 | 19.97 |
| ATOM | 20396 | OH2 | WAT | 3466 | 41.165 | 1.509 | 54.227 | 1.00 | 20.56 |
| ATOM | 20397 | OH2 | WAT | 3467 | 34.145 | −48.601 | 22.561 | 1.00 | 23.68 |
| ATOM | 20398 | OH2 | WAT | 3468 | 14.133 | −2.489 | −25.544 | 1.00 | 32.11 |
| ATOM | 20399 | OH2 | WAT | 3469 | −1.808 | −41.623 | 29.404 | 1.00 | 23.41 |
| ATOM | 20400 | OH2 | WAT | 3470 | 37.724 | −31.627 | 30.385 | 1.00 | 31.57 |
| ATOM | 20401 | OH2 | WAT | 3471 | 14.985 | −55.814 | 12.662 | 1.00 | 21.80 |
| ATOM | 20402 | OH2 | WAT | 3472 | 24.908 | −25.348 | 43.709 | 1.00 | 20.59 |
| ATOM | 20403 | OH2 | WAT | 3473 | 25.698 | −49.669 | 27.512 | 1.00 | 22.17 |
| ATOM | 20404 | OH2 | WAT | 3474 | 17.811 | −17.127 | 39.808 | 1.00 | 17.19 |
| ATOM | 20405 | OH2 | WAT | 3475 | 21.718 | 14.837 | 7.814 | 1.00 | 24.90 |
| ATOM | 20406 | OH2 | WAT | 3476 | 22.441 | −3.352 | 58.930 | 1.00 | 24.81 |
| ATOM | 20407 | OH2 | WAT | 3477 | 24.448 | 30.315 | 43.032 | 1.00 | 26.95 |
| ATOM | 20408 | OH2 | WAT | 3478 | 44.123 | 12.038 | 19.936 | 1.00 | 31.08 |
| ATOM | 20409 | OH2 | WAT | 3479 | 20.286 | −30.479 | 61.570 | 1.00 | 20.29 |
| ATOM | 20410 | OH2 | WAT | 3480 | 38.326 | −19.073 | 41.512 | 1.00 | 22.18 |
| ATOM | 20411 | OH2 | WAT | 3481 | −14.815 | 27.992 | 61.688 | 1.00 | 40.66 |
| ATOM | 20412 | OH2 | WAT | 3482 | 34.625 | −26.878 | 39.822 | 1.00 | 33.03 |
| ATOM | 20413 | OH2 | WAT | 3483 | −3.096 | 35.761 | 7.054 | 1.00 | 38.60 |
| ATOM | 20414 | OH2 | WAT | 3484 | 9.956 | −32.525 | 39.001 | 1.00 | 30.29 |
| ATOM | 20415 | OH2 | WAT | 3485 | 4.975 | −31.893 | 10.831 | 1.00 | 21.28 |
| ATOM | 20416 | OH2 | WAT | 3486 | 17.184 | −13.200 | −22.519 | 1.00 | 28.57 |
| ATOM | 20417 | OH2 | WAT | 3487 | 7.504 | 28.777 | 40.806 | 1.00 | 31.23 |
| ATOM | 20418 | OH2 | WAT | 3488 | −9.436 | 22.775 | 21.943 | 1.00 | 18.82 |
| ATOM | 20419 | OH2 | WAT | 3489 | 29.474 | 3.455 | 1.487 | 1.00 | 32.63 |
| ATOM | 20420 | OH2 | WAT | 3490 | 0.602 | 20.241 | 7.100 | 1.00 | 22.23 |
| ATOM | 20421 | OH2 | WAT | 3491 | 9.850 | −7.937 | 5.724 | 1.00 | 28.64 |
| ATOM | 20422 | OH2 | WAT | 3492 | 32.489 | −21.034 | −10.996 | 1.00 | 30.63 |
| ATOM | 20423 | OH2 | WAT | 3493 | 7.930 | −16.894 | 18.890 | 1.00 | 20.22 |
| ATOM | 20424 | OH2 | WAT | 3494 | −4.506 | −24.196 | 3.635 | 1.00 | 18.30 |
| ATOM | 20425 | OH2 | WAT | 3495 | 10.221 | −19.687 | 36.867 | 1.00 | 24.52 |
| ATOM | 20426 | OH2 | WAT | 3496 | −1.557 | 19.729 | 17.609 | 1.00 | 19.45 |
| ATOM | 20427 | OH2 | WAT | 3497 | −29.812 | −4.805 | 64.317 | 1.00 | 19.66 |
| ATOM | 20428 | OH2 | WAT | 3498 | −26.364 | 8.727 | −2.492 | 1.00 | 23.78 |
| ATOM | 20429 | OH2 | WAT | 3499 | 3.181 | −15.486 | 54.400 | 1.00 | 26.52 |
| ATOM | 20430 | OH2 | WAT | 3500 | −4.211 | 28.797 | 70.359 | 1.00 | 29.23 |
| ATOM | 20431 | OH2 | WAT | 3501 | 23.091 | −1.819 | −22.141 | 1.00 | 27.87 |
| ATOM | 20432 | OH2 | WAT | 3502 | 53.226 | 7.462 | 46.155 | 1.00 | 28.40 |
| ATOM | 20433 | OH2 | WAT | 3503 | 47.327 | 21.844 | 54.130 | 1.00 | 25.03 |
| ATOM | 20434 | OH2 | WAT | 3504 | −7.836 | 15.601 | 26.509 | 1.00 | 22.64 |
| ATOM | 20435 | OH2 | WAT | 3505 | 4.797 | −16.467 | −3.276 | 1.00 | 30.07 |
| ATOM | 20436 | OH2 | WAT | 3506 | 31.664 | −6.522 | −13.027 | 1.00 | 22.55 |
| ATOM | 20437 | OH2 | WAT | 3507 | 21.645 | 31.075 | 60.847 | 1.00 | 17.96 |
| ATOM | 20438 | OH2 | WAT | 3508 | 14.391 | 8.824 | 50.006 | 1.00 | 23.64 |
| ATOM | 20439 | OH2 | WAT | 3509 | −17.132 | 4.640 | 49.766 | 1.00 | 23.89 |
| ATOM | 20440 | OH2 | WAT | 3510 | 6.062 | −26.464 | 52.738 | 1.00 | 30.15 |
| ATOM | 20441 | OH2 | WAT | 3511 | 5.200 | 26.208 | 12.471 | 1.00 | 34.21 |
| ATOM | 20442 | OH2 | WAT | 3512 | 16.071 | 0.370 | −25.723 | 1.00 | 33.79 |
| ATOM | 20443 | OH2 | WAT | 3513 | 32.797 | −0.260 | 48.448 | 1.00 | 20.41 |
| ATOM | 20444 | OH2 | WAT | 3514 | −16.216 | 1.768 | 0.259 | 1.00 | 23.90 |
| ATOM | 20445 | OH2 | WAT | 3515 | −5.097 | 2.007 | 1.772 | 1.00 | 19.02 |
| ATOM | 20446 | OH2 | WAT | 3516 | −14.399 | −28.368 | 53.744 | 1.00 | 43.61 |
| ATOM | 20447 | OH2 | WAT | 3517 | 1.256 | −15.041 | 9.961 | 1.00 | 24.99 |
| ATOM | 20448 | OH2 | WAT | 3518 | 29.943 | 12.201 | 27.604 | 1.00 | 32.31 |
| ATOM | 20449 | OH2 | WAT | 3519 | 20.357 | 22.467 | 26.147 | 1.00 | 24.34 |
| ATOM | 20450 | OH2 | WAT | 3520 | 11.136 | −14.234 | 75.415 | 1.00 | 25.64 |
| ATOM | 20451 | OH2 | WAT | 3521 | 38.180 | −15.356 | 56.012 | 1.00 | 22.35 |
| ATOM | 20452 | OH2 | WAT | 3522 | 24.649 | −24.017 | 2.168 | 1.00 | 29.47 |
| ATOM | 20453 | OH2 | WAT | 3523 | 36.842 | 8.360 | 26.719 | 1.00 | 24.17 |
| ATOM | 20454 | OH2 | WAT | 3524 | −30.258 | −8.726 | 23.554 | 1.00 | 27.51 |
| ATOM | 20455 | OH2 | WAT | 3525 | 17.453 | −26.604 | 11.072 | 1.00 | 24.69 |
| ATOM | 20456 | OH2 | WAT | 3526 | −0.804 | −51.986 | 25.853 | 1.00 | 26.03 |
| ATOM | 20457 | OH2 | WAT | 3527 | −9.459 | −21.976 | 39.845 | 1.00 | 29.39 |
| ATOM | 20458 | OH2 | WAT | 3528 | 29.511 | −4.884 | 16.485 | 1.00 | 27.39 |
| ATOM | 20459 | OH2 | WAT | 3529 | 14.158 | −1.502 | 9.556 | 1.00 | 35.97 |
| ATOM | 20460 | OH2 | WAT | 3530 | −14.703 | 20.404 | 38.717 | 1.00 | 28.71 |
| ATOM | 20461 | OH2 | WAT | 3531 | −7.105 | 1.156 | −13.754 | 1.00 | 18.83 |
| ATOM | 20462 | OH2 | WAT | 3532 | 21.848 | −21.505 | 48.844 | 1.00 | 27.48 |
| ATOM | 20463 | OH2 | WAT | 3533 | 36.224 | −2.915 | 32.963 | 1.00 | 27.24 |
| ATOM | 20464 | OH2 | WAT | 3534 | 23.251 | 23.615 | 68.702 | 1.00 | 18.75 |
| ATOM | 20465 | OH2 | WAT | 3535 | −13.136 | −41.931 | 8.140 | 1.00 | 22.80 |
| ATOM | 20466 | OH2 | WAT | 3536 | −3.662 | 29.839 | 18.235 | 1.00 | 22.35 |
| ATOM | 20467 | OH2 | WAT | 3537 | 42.444 | −25.120 | 50.779 | 1.00 | 30.29 |
| ATOM | 20468 | OH2 | WAT | 3538 | 30.684 | 13.800 | 60.308 | 1.00 | 26.04 |
| ATOM | 20469 | OH2 | WAT | 3539 | −1.141 | 19.587 | 14.828 | 1.00 | 19.25 |
| ATOM | 20470 | OH2 | WAT | 3540 | −5.566 | −28.227 | 31.527 | 1.00 | 31.13 |
| ATOM | 20471 | OH2 | WAT | 3541 | 37.483 | −2.016 | 49.590 | 1.00 | 23.36 |
| ATOM | 20472 | OH2 | WAT | 3542 | −4.059 | −1.055 | −0.721 | 1.00 | 31.71 |
| ATOM | 20473 | OH2 | WAT | 3543 | 43.257 | −3.965 | 48.382 | 1.00 | 24.84 |
| ATOM | 20474 | OH2 | WAT | 3544 | 16.003 | 15.425 | 17.335 | 1.00 | 27.51 |
| ATOM | 20475 | OH2 | WAT | 3545 | −12.199 | 21.137 | 35.789 | 1.00 | 24.22 |
| ATOM | 20476 | OH2 | WAT | 3546 | 43.270 | 11.938 | 40.176 | 1.00 | 30.17 |
| ATOM | 20477 | OH2 | WAT | 3547 | 8.127 | −56.905 | 24.805 | 1.00 | 19.10 |
| ATOM | 20478 | OH2 | WAT | 3548 | 20.613 | 33.154 | 62.310 | 1.00 | 30.19 |
| ATOM | 20479 | OH2 | WAT | 3549 | 25.111 | 15.458 | 30.152 | 1.00 | 22.79 |
| ATOM | 20480 | OH2 | WAT | 3550 | 45.070 | 2.400 | 26.630 | 1.00 | 30.97 |
| ATOM | 20481 | OH2 | WAT | 3551 | 2.048 | −27.128 | 63.747 | 1.00 | 22.51 |
| ATOM | 20482 | OH2 | WAT | 3552 | −5.000 | 1.632 | −22.794 | 1.00 | 28.73 |
| ATOM | 20483 | OH2 | WAT | 3553 | 40.494 | −30.756 | 61.314 | 1.00 | 31.19 |
| ATOM | 20484 | OH2 | WAT | 3554 | 21.635 | 29.643 | 51.600 | 1.00 | 24.47 |
| ATOM | 20485 | OH2 | WAT | 3555 | 20.234 | 18.268 | 52.367 | 1.00 | 25.61 |
| ATOM | 20486 | OH2 | WAT | 3556 | 6.420 | 8.840 | 15.848 | 1.00 | 17.10 |
| ATOM | 20487 | OH2 | WAT | 3557 | −23.960 | −31.184 | 20.786 | 1.00 | 23.74 |
| ATOM | 20488 | OH2 | WAT | 3558 | 3.694 | −28.168 | 52.744 | 1.00 | 28.76 |
| ATOM | 20489 | OH2 | WAT | 3559 | −0.279 | 24.388 | 69.202 | 1.00 | 29.63 |
| ATOM | 20490 | OH2 | WAT | 3560 | −13.233 | −14.029 | 5.054 | 1.00 | 30.16 |
| ATOM | 20491 | OH2 | WAT | 3561 | −11.730 | 2.751 | 48.422 | 1.00 | 23.44 |
| ATOM | 20492 | OH2 | WAT | 3562 | 25.841 | −1.986 | 41.378 | 1.00 | 30.71 |
| ATOM | 20493 | OH2 | WAT | 3563 | −19.840 | 9.517 | 11.289 | 1.00 | 30.98 |
| ATOM | 20494 | OH2 | WAT | 3564 | 35.901 | −16.807 | 38.350 | 1.00 | 23.60 |
| ATOM | 20495 | OH2 | WAT | 3565 | 5.279 | 14.511 | 23.101 | 1.00 | 32.53 |
| ATOM | 20496 | OH2 | WAT | 3566 | −12.669 | 24.543 | 21.588 | 1.00 | 27.87 |
| ATOM | 20497 | OH2 | WAT | 3567 | 2.422 | 29.279 | 73.739 | 1.00 | 26.24 |
| ATOM | 20498 | OH2 | WAT | 3568 | 24.933 | 2.790 | 38.157 | 1.00 | 31.71 |
| ATOM | 20499 | OH2 | WAT | 3569 | −6.126 | 31.127 | 44.730 | 1.00 | 24.66 |
| ATOM | 20500 | OH2 | WAT | 3570 | 12.732 | −49.375 | 29.754 | 1.00 | 24.42 |
| ATOM | 20501 | OH2 | WAT | 3571 | 54.364 | −4.263 | 57.668 | 1.00 | 28.75 |
| ATOM | 20502 | OH2 | WAT | 3572 | 8.524 | 5.432 | 51.993 | 1.00 | 29.48 |
| ATOM | 20503 | OH2 | WAT | 3573 | 2.845 | −17.320 | 27.601 | 1.00 | 23.94 |
| ATOM | 20504 | OH2 | WAT | 3574 | −16.502 | 21.486 | −18.054 | 1.00 | 27.10 |
| ATOM | 20505 | OH2 | WAT | 3575 | 43.324 | 21.268 | 62.048 | 1.00 | 21.29 |
| ATOM | 20506 | OH2 | WAT | 3576 | −13.809 | −17.874 | 23.626 | 1.00 | 21.22 |
| ATOM | 20507 | OH2 | WAT | 3577 | 6.416 | −10.418 | 67.564 | 1.00 | 25.45 |
| ATOM | 20508 | OH2 | WAT | 3578 | 23.420 | −23.177 | 10.075 | 1.00 | 31.50 |
| ATOM | 20509 | OH2 | WAT | 3579 | 18.146 | 5.699 | 42.569 | 1.00 | 27.60 |
| ATOM | 20510 | OH2 | WAT | 3580 | 10.508 | 14.399 | 39.740 | 1.00 | 21.36 |
| ATOM | 20511 | OH2 | WAT | 3581 | 0.784 | −6.629 | 15.625 | 1.00 | 15.08 |
| ATOM | 20512 | OH2 | WAT | 3582 | 5.497 | −17.436 | 23.138 | 1.00 | 25.09 |
| ATOM | 20513 | OH2 | WAT | 3583 | 7.991 | 6.071 | 43.749 | 1.00 | 15.60 |
| ATOM | 20514 | OH2 | WAT | 3584 | 3.189 | −11.871 | 38.573 | 1.00 | 16.47 |
| ATOM | 20515 | OH2 | WAT | 3585 | −0.207 | −12.212 | 31.665 | 1.00 | 17.46 |
| ATOM | 20516 | OH2 | WAT | 3586 | 13.002 | −15.561 | 35.959 | 1.00 | 21.13 |
| ATOM | 20517 | OH2 | WAT | 3587 | 20.589 | 15.736 | 21.973 | 1.00 | 26.75 |
| ATOM | 20518 | OH2 | WAT | 3588 | 21.090 | 3.770 | 13.740 | 1.00 | 22.84 |
| ATOM | 20519 | OH2 | WAT | 3589 | 9.607 | 17.572 | 31.827 | 1.00 | 19.08 |
| ATOM | 20520 | OH2 | WAT | 3590 | 0.276 | −14.520 | 35.798 | 1.00 | 20.57 |
| ATOM | 20521 | OH2 | WAT | 3591 | −13.859 | 19.020 | −13.607 | 1.00 | 20.14 |
| ATOM | 20522 | OH2 | WAT | 3592 | 22.420 | 11.927 | 35.726 | 1.00 | 18.41 |
| ATOM | 20523 | OH2 | WAT | 3593 | −3.566 | −2.525 | −6.831 | 1.00 | 34.12 |
| ATOM | 20524 | OH2 | WAT | 3594 | 24.320 | 2.280 | 42.362 | 1.00 | 24.09 |
| ATOM | 20525 | OH2 | WAT | 3595 | 19.088 | 0.614 | 44.019 | 1.00 | 22.35 |
| ATOM | 20526 | OH2 | WAT | 3596 | 15.248 | 1.171 | −17.886 | 1.00 | 22.97 |
| ATOM | 20527 | OH2 | WAT | 3597 | 24.372 | 9.943 | 24.127 | 1.00 | 21.06 |
| ATOM | 20528 | OH2 | WAT | 3598 | 16.764 | −1.795 | 47.475 | 1.00 | 21.98 |
| ATOM | 20529 | OH2 | WAT | 3599 | 0.414 | −0.352 | 12.854 | 1.00 | 18.76 |
| ATOM | 20530 | OH2 | WAT | 3600 | −4.201 | −10.546 | 27.179 | 1.00 | 23.37 |
| ATOM | 20531 | OH2 | WAT | 3601 | 6.884 | −2.275 | 45.268 | 1.00 | 28.10 |
| ATOM | 20532 | OH2 | WAT | 3602 | 23.685 | 15.179 | 34.059 | 1.00 | 22.99 |
| ATOM | 20533 | OH2 | WAT | 3603 | 2.551 | −10.373 | 13.582 | 1.00 | 21.06 |
| ATOM | 20534 | OH2 | WAT | 3604 | 21.695 | 8.786 | 16.503 | 1.00 | 23.43 |
| ATOM | 20535 | OH2 | WAT | 3605 | 12.902 | 6.151 | 48.325 | 1.00 | 21.73 |
| ATOM | 20536 | OH2 | WAT | 3606 | 9.189 | 5.244 | 48.426 | 1.00 | 21.63 |
| ATOM | 20537 | OH2 | WAT | 3607 | 2.378 | −10.193 | 17.669 | 1.00 | 16.25 |
| ATOM | 20538 | OH2 | WAT | 3608 | 4.820 | −14.212 | 23.885 | 1.00 | 30.32 |
| ATOM | 20539 | OH2 | WAT | 3609 | 42.612 | 26.598 | 65.519 | 1.00 | 33.56 |
| ATOM | 20540 | OH2 | WAT | 3610 | 12.232 | −7.077 | 44.497 | 1.00 | 15.58 |
| ATOM | 20541 | OH2 | WAT | 3611 | 11.183 | −17.225 | 30.756 | 1.00 | 22.33 |
| ATOM | 20542 | OH2 | WAT | 3612 | 34.209 | −6.592 | −13.887 | 1.00 | 24.62 |
| ATOM | 20543 | OH2 | WAT | 3613 | 6.463 | 24.230 | 7.734 | 1.00 | 20.77 |
| ATOM | 20544 | OH2 | WAT | 3614 | 33.223 | −2.006 | 8.282 | 1.00 | 23.63 |
| ATOM | 20545 | OH2 | WAT | 3615 | −11.992 | −21.341 | 31.338 | 1.00 | 21.59 |
| ATOM | 20546 | OH2 | WAT | 3616 | 20.404 | −27.602 | 62.377 | 1.00 | 21.88 |
| ATOM | 20547 | OH2 | WAT | 3617 | 10.020 | −10.313 | 43.379 | 1.00 | 30.16 |
| ATOM | 20548 | OH2 | WAT | 3618 | 10.356 | −15.159 | 17.815 | 1.00 | 21.94 |
| ATOM | 20549 | OH2 | WAT | 3619 | 1.389 | −11.017 | 36.934 | 1.00 | 20.03 |
| ATOM | 20550 | OH2 | WAT | 3620 | 4.363 | −14.468 | 52.055 | 1.00 | 24.43 |
| ATOM | 20551 | OH2 | WAT | 3621 | 27.484 | 2.764 | 55.962 | 1.00 | 21.15 |
| ATOM | 20552 | OH2 | WAT | 3622 | 24.726 | −24.186 | 16.875 | 1.00 | 24.05 |
| ATOM | 20553 | OH2 | WAT | 3623 | 0.014 | −20.733 | 40.658 | 1.00 | 16.17 |
| ATOM | 20554 | OH2 | WAT | 3624 | 2.725 | 4.728 | 52.388 | 1.00 | 26.67 |
| ATOM | 20555 | OH2 | WAT | 3625 | 9.746 | −14.612 | 38.887 | 1.00 | 21.86 |
| ATOM | 20556 | OH2 | WAT | 3626 | −4.757 | −1.899 | 1.970 | 1.00 | 35.13 |
| ATOM | 20557 | OH2 | WAT | 3627 | 16.835 | 4.432 | 45.116 | 1.00 | 18.72 |
| ATOM | 20558 | OH2 | WAT | 3628 | 12.251 | −47.696 | 7.896 | 1.00 | 22.73 |
| ATOM | 20559 | OH2 | WAT | 3629 | 39.019 | −10.307 | 16.860 | 1.00 | 20.92 |
| ATOM | 20560 | OH2 | WAT | 3630 | −15.949 | −40.710 | 24.697 | 1.00 | 22.11 |
| ATOM | 20561 | OH2 | WAT | 3631 | 25.811 | −15.143 | 32.335 | 1.00 | 27.41 |
| ATOM | 20562 | OH2 | WAT | 3632 | 15.761 | −3.942 | 44.388 | 1.00 | 18.59 |
| ATOM | 20563 | OH2 | WAT | 3633 | 31.978 | −22.985 | 68.623 | 1.00 | 27.31 |
| ATOM | 20564 | OH2 | WAT | 3634 | −26.509 | −0.176 | 42.412 | 1.00 | 18.54 |
| ATOM | 20565 | OH2 | WAT | 3635 | −15.520 | −42.723 | 20.234 | 1.00 | 26.20 |
| ATOM | 20566 | OH2 | WAT | 3636 | 12.167 | −11.989 | 10.440 | 1.00 | 19.28 |
| ATOM | 20567 | OH2 | WAT | 3637 | −4.154 | −47.994 | 12.332 | 1.00 | 35.26 |
| ATOM | 20568 | OH2 | WAT | 3638 | −1.615 | 3.195 | 59.259 | 1.00 | 25.77 |
| ATOM | 20569 | OH2 | WAT | 3639 | −9.493 | −3.891 | 48.064 | 1.00 | 28.70 |
| ATOM | 20570 | OH2 | WAT | 3640 | 16.898 | 8.348 | −11.925 | 1.00 | 34.50 |
| ATOM | 20571 | OH2 | WAT | 3641 | 32.002 | −14.281 | −16.376 | 1.00 | 28.21 |
| ATOM | 20572 | OH2 | WAT | 3642 | −2.196 | −11.144 | 8.403 | 1.00 | 24.46 |
| ATOM | 20573 | OH2 | WAT | 3643 | 29.916 | 22.387 | 32.574 | 1.00 | 37.43 |
| ATOM | 20574 | OH2 | WAT | 3644 | 18.608 | 21.247 | 53.393 | 1.00 | 17.98 |
| ATOM | 20575 | OH2 | WAT | 3645 | 38.976 | −31.818 | 26.231 | 1.00 | 30.85 |
| ATOM | 20576 | OH2 | WAT | 3646 | 20.552 | −7.819 | 18.900 | 1.00 | 25.27 |
| ATOM | 20577 | OH2 | WAT | 3647 | 12.060 | 9.642 | 9.127 | 1.00 | 22.18 |
| ATOM | 20578 | OH2 | WAT | 3648 | −31.694 | −12.599 | 11.058 | 1.00 | 34.99 |
| ATOM | 20579 | OH2 | WAT | 3649 | 37.537 | −7.544 | 19.487 | 1.00 | 26.06 |
| ATOM | 20580 | OH2 | WAT | 3650 | 0.189 | −29.389 | 54.461 | 1.00 | 23.97 |
| ATOM | 20581 | OH2 | WAT | 3651 | −5.852 | −18.327 | 36.510 | 1.00 | 26.63 |
| ATOM | 20582 | OH2 | WAT | 3652 | 14.864 | −9.336 | 13.735 | 1.00 | 20.83 |
| ATOM | 20583 | OH2 | WAT | 3653 | −12.948 | 3.519 | −18.993 | 1.00 | 25.64 |
| ATOM | 20584 | OH2 | WAT | 3654 | −12.613 | 2.553 | 24.159 | 1.00 | 22.97 |
| ATOM | 20585 | OH2 | WAT | 3655 | 25.889 | −19.104 | −21.194 | 1.00 | 24.77 |
| ATOM | 20586 | OH2 | WAT | 3656 | 31.173 | −1.987 | 20.477 | 1.00 | 24.59 |
| ATOM | 20587 | OH2 | WAT | 3657 | 40.085 | −39.316 | 22.789 | 1.00 | 28.48 |
| ATOM | 20588 | OH2 | WAT | 3658 | 3.870 | −32.022 | 8.417 | 1.00 | 26.65 |
| ATOM | 20589 | OH2 | WAT | 3659 | 18.544 | −28.551 | 16.087 | 1.00 | 34.40 |
| ATOM | 20590 | OH2 | WAT | 3660 | −6.861 | −27.077 | 47.127 | 1.00 | 22.70 |
| ATOM | 20591 | OH2 | WAT | 3661 | 13.344 | −12.961 | 12.757 | 1.00 | 21.21 |
| ATOM | 20592 | OH2 | WAT | 3662 | 23.924 | 13.058 | 30.417 | 1.00 | 26.23 |
| ATOM | 20593 | OH2 | WAT | 3663 | −16.463 | 0.666 | 27.530 | 1.00 | 23.58 |
| ATOM | 20594 | OH2 | WAT | 3664 | 29.982 | −28.850 | 48.332 | 1.00 | 25.57 |
| ATOM | 20595 | OH2 | WAT | 3665 | −17.510 | 7.033 | 13.800 | 1.00 | 22.45 |
| ATOM | 20596 | OH2 | WAT | 3666 | 1.881 | −15.322 | 29.876 | 1.00 | 24.08 |
| ATOM | 20597 | OH2 | WAT | 3667 | 6.507 | −5.396 | 11.525 | 1.00 | 29.49 |
| ATOM | 20598 | OH2 | WAT | 3668 | 46.905 | 1.491 | 23.229 | 1.00 | 29.41 |
| ATOM | 20599 | OH2 | WAT | 3669 | −32.090 | −8.170 | 19.323 | 1.00 | 30.01 |
| ATOM | 20600 | OH2 | WAT | 3670 | 22.927 | −19.385 | 47.561 | 1.00 | 22.37 |
| ATOM | 20601 | OH2 | WAT | 3671 | −22.132 | 17.236 | 0.786 | 1.00 | 28.38 |
| ATOM | 20602 | OH2 | WAT | 3672 | 33.600 | −44.658 | 11.611 | 1.00 | 34.36 |
| ATOM | 20603 | OH2 | WAT | 3673 | −13.806 | 7.350 | 36.529 | 1.00 | 29.31 |
| ATOM | 20604 | OH2 | WAT | 3674 | −27.433 | −25.257 | 7.569 | 1.00 | 36.53 |
| ATOM | 20605 | OH2 | WAT | 3675 | −8.731 | −1.580 | 30.817 | 1.00 | 29.08 |
| ATOM | 20606 | OH2 | WAT | 3676 | 20.432 | −3.056 | 56.541 | 1.00 | 26.44 |
| ATOM | 20607 | OH2 | WAT | 3677 | 3.370 | −13.090 | 13.599 | 1.00 | 17.38 |
| ATOM | 20608 | OH2 | WAT | 3678 | 8.012 | −34.825 | 12.677 | 1.00 | 31.51 |
| ATOM | 20609 | OH2 | WAT | 3679 | 1.072 | −19.415 | 5.239 | 1.00 | 20.20 |
| ATOM | 20610 | OH2 | WAT | 3680 | 21.321 | 8.443 | 12.592 | 1.00 | 22.90 |
| ATOM | 20611 | OH2 | WAT | 3681 | 52.644 | 0.777 | 61.381 | 1.00 | 35.77 |
| ATOM | 20612 | OH2 | WAT | 3682 | −3.097 | −41.815 | 7.665 | 1.00 | 31.57 |
| ATOM | 20613 | OH2 | WAT | 3683 | 20.159 | −13.750 | 38.154 | 1.00 | 29.71 |
| ATOM | 20614 | OH2 | WAT | 3684 | −9.685 | 15.081 | 52.281 | 1.00 | 25.91 |
| ATOM | 20615 | OH2 | WAT | 3685 | 28.135 | 20.650 | 0.860 | 1.00 | 27.21 |
| ATOM | 20616 | OH2 | WAT | 3686 | 39.001 | −41.238 | 18.806 | 1.00 | 26.62 |
| ATOM | 20617 | OH2 | WAT | 3687 | 40.023 | −7.002 | 42.151 | 1.00 | 26.60 |
| ATOM | 20618 | OH2 | WAT | 3688 | −17.015 | 2.383 | 5.482 | 1.00 | 22.76 |
| ATOM | 20619 | OH2 | WAT | 3689 | 31.736 | −26.387 | 15.172 | 1.00 | 27.64 |
| ATOM | 20620 | OH2 | WAT | 3690 | 37.471 | −8.993 | −17.582 | 1.00 | 30.40 |
| ATOM | 20621 | OH2 | WAT | 3691 | 10.812 | 10.675 | 11.247 | 1.00 | 23.49 |
| ATOM | 20622 | OH2 | WAT | 3692 | 36.504 | −25.533 | 25.441 | 1.00 | 23.11 |
| ATOM | 20623 | OH2 | WAT | 3693 | 22.254 | −32.457 | 61.678 | 1.00 | 26.40 |
| ATOM | 20624 | OH2 | WAT | 3694 | 11.931 | −14.704 | 7.800 | 1.00 | 21.35 |
| ATOM | 20625 | OH2 | WAT | 3695 | −23.681 | −14.788 | 45.643 | 1.00 | 24.78 |
| ATOM | 20626 | OH2 | WAT | 3696 | 7.182 | −51.466 | 28.627 | 1.00 | 25.44 |
| ATOM | 20627 | OH2 | WAT | 3697 | −22.958 | −2.211 | 23.235 | 1.00 | 26.83 |
| ATOM | 20628 | OH2 | WAT | 3698 | 32.975 | −23.395 | −0.121 | 1.00 | 22.51 |
| ATOM | 20629 | OH2 | WAT | 3699 | −4.858 | 3.626 | 3.892 | 1.00 | 28.24 |
| ATOM | 20630 | OH2 | WAT | 3700 | −25.802 | −17.472 | 57.336 | 1.00 | 24.02 |
| ATOM | 20631 | OH2 | WAT | 3701 | −24.961 | −8.029 | 27.571 | 1.00 | 30.46 |
| ATOM | 20632 | OH2 | WAT | 3702 | −19.843 | 6.476 | 47.329 | 1.00 | 25.78 |
| ATOM | 20633 | OH2 | WAT | 3703 | −22.522 | 14.871 | 2.427 | 1.00 | 24.66 |
| ATOM | 20634 | OH2 | WAT | 3704 | 18.502 | 41.801 | 32.590 | 1.00 | 31.05 |
| ATOM | 20635 | OH2 | WAT | 3705 | −2.285 | 23.880 | −16.394 | 1.00 | 41.88 |
| ATOM | 20636 | OH2 | WAT | 3706 | 9.076 | 7.121 | 12.265 | 1.00 | 24.55 |
| ATOM | 20637 | OH2 | WAT | 3707 | 22.472 | 16.738 | 36.131 | 1.00 | 28.80 |
| ATOM | 20638 | OH2 | WAT | 3708 | 23.106 | 19.524 | 28.362 | 1.00 | 29.35 |
| ATOM | 20639 | OH2 | WAT | 3709 | 20.660 | −1.685 | 53.178 | 1.00 | 28.07 |
| ATOM | 20640 | OH2 | WAT | 3710 | 47.414 | −15.667 | 4.341 | 1.00 | 26.85 |
| ATOM | 20641 | OH2 | WAT | 3711 | 35.787 | −27.874 | 44.447 | 1.00 | 27.28 |
| ATOM | 20642 | OH2 | WAT | 3712 | −9.662 | 17.345 | 32.733 | 1.00 | 23.43 |
| ATOM | 20643 | OH2 | WAT | 3713 | −23.756 | −18.816 | 55.654 | 1.00 | 27.14 |
| ATOM | 20644 | OH2 | WAT | 3714 | 26.158 | −26.333 | 67.093 | 1.00 | 25.71 |
| ATOM | 20645 | OH2 | WAT | 3715 | 8.560 | 15.732 | 30.041 | 1.00 | 29.85 |
| ATOM | 20646 | OH2 | WAT | 3716 | 47.272 | 25.719 | 59.342 | 1.00 | 32.35 |
| ATOM | 20647 | OH2 | WAT | 3717 | 17.487 | −9.851 | −23.543 | 1.00 | 38.83 |
| ATOM | 20648 | OH2 | WAT | 3718 | −18.150 | −9.422 | 11.110 | 1.00 | 24.14 |
| ATOM | 20649 | OH2 | WAT | 3719 | 28.787 | −33.914 | 15.756 | 1.00 | 21.48 |
| ATOM | 20650 | OH2 | WAT | 3720 | −2.825 | 9.854 | 58.691 | 1.00 | 29.62 |
| ATOM | 20651 | OH2 | WAT | 3721 | 35.002 | −40.084 | 48.371 | 1.00 | 34.58 |
| ATOM | 20652 | OH2 | WAT | 3722 | 33.278 | −36.251 | 12.757 | 1.00 | 26.17 |
| ATOM | 20653 | OH2 | WAT | 3723 | 7.979 | −20.213 | 33.159 | 1.00 | 18.19 |
| ATOM | 20654 | OH2 | WAT | 3724 | −18.398 | 28.206 | 46.922 | 1.00 | 33.34 |
| ATOM | 20655 | OH2 | WAT | 3725 | 14.111 | 10.971 | 39.946 | 1.00 | 39.42 |
| ATOM | 20656 | OH2 | WAT | 3726 | 20.899 | −23.856 | 76.684 | 1.00 | 26.04 |
| ATOM | 20657 | OH2 | WAT | 3727 | 12.600 | −30.532 | 39.040 | 1.00 | 49.07 |
| ATOM | 20658 | OH2 | WAT | 3728 | 0.214 | −28.712 | 62.508 | 1.00 | 28.93 |
| ATOM | 20659 | OH2 | WAT | 3729 | 29.212 | −6.590 | 4.979 | 1.00 | 24.84 |
| ATOM | 20660 | OH2 | WAT | 3730 | −17.997 | 34.615 | 48.280 | 1.00 | 28.78 |
| ATOM | 20661 | OH2 | WAT | 3731 | 21.283 | 23.102 | 66.907 | 1.00 | 26.75 |
| ATOM | 20662 | OH2 | WAT | 3732 | 6.942 | −10.436 | 70.783 | 1.00 | 30.98 |
| ATOM | 20663 | OH2 | WAT | 3733 | −13.774 | 4.431 | 49.066 | 1.00 | 24.84 |
| ATOM | 20664 | OH2 | WAT | 3734 | 27.594 | 0.198 | 38.138 | 1.00 | 33.10 |
| ATOM | 20665 | OH2 | WAT | 3735 | 20.794 | 11.104 | 12.321 | 1.00 | 27.55 |
| ATOM | 20666 | OH2 | WAT | 3736 | 51.256 | 16.589 | 59.899 | 1.00 | 30.84 |
| ATOM | 20667 | OH2 | WAT | 3737 | 22.183 | −14.735 | 24.243 | 1.00 | 28.02 |
| ATOM | 20668 | OH2 | WAT | 3738 | 44.096 | −11.703 | 13.054 | 1.00 | 28.23 |
| ATOM | 20669 | OH2 | WAT | 3739 | 18.453 | −23.992 | 49.772 | 1.00 | 32.67 |
| ATOM | 20670 | OH2 | WAT | 3740 | 10.967 | 16.592 | 34.278 | 1.00 | 31.80 |
| ATOM | 20671 | OH2 | WAT | 3741 | −9.858 | 9.563 | 20.327 | 1.00 | 20.75 |
| ATOM | 20672 | OH2 | WAT | 3742 | 0.898 | 42.404 | 5.444 | 1.00 | 33.16 |
| ATOM | 20673 | OH2 | WAT | 3743 | 3.933 | −29.127 | 37.039 | 1.00 | 23.75 |
| ATOM | 20674 | OH2 | WAT | 3744 | 36.228 | −24.732 | 33.177 | 1.00 | 44.37 |
| ATOM | 20675 | OH2 | WAT | 3745 | 3.159 | 7.122 | 37.485 | 1.00 | 29.60 |
| ATOM | 20676 | OH2 | WAT | 3746 | −10.921 | 2.269 | 51.233 | 1.00 | 27.65 |
| ATOM | 20677 | OH2 | WAT | 3747 | 53.348 | 17.944 | 49.378 | 1.00 | 27.14 |
| ATOM | 20678 | OH2 | WAT | 3748 | 7.606 | 22.178 | 51.653 | 1.00 | 40.12 |
| ATOM | 20679 | OH2 | WAT | 3749 | 39.204 | −34.717 | 27.730 | 1.00 | 24.03 |
| ATOM | 20680 | OH2 | WAT | 3750 | 13.564 | 19.874 | 31.976 | 1.00 | 23.45 |
| ATOM | 20681 | OH2 | WAT | 3751 | −3.814 | −23.747 | 36.732 | 1.00 | 26.02 |
| ATOM | 20682 | OH2 | WAT | 3752 | 47.146 | 16.831 | 44.337 | 1.00 | 26.65 |
| ATOM | 20683 | OH2 | WAT | 3753 | 13.930 | −30.419 | 44.084 | 1.00 | 25.81 |
| ATOM | 20684 | OH2 | WAT | 3754 | 9.861 | −9.263 | 8.727 | 1.00 | 30.58 |
| ATOM | 20685 | OH2 | WAT | 3755 | 23.683 | 32.606 | 59.197 | 1.00 | 32.94 |
| ATOM | 20686 | OH2 | WAT | 3756 | 20.056 | 31.032 | 18.101 | 1.00 | 36.08 |
| ATOM | 20687 | OH2 | WAT | 3757 | −10.879 | 29.267 | 63.971 | 1.00 | 35.86 |
| ATOM | 20688 | OH2 | WAT | 3758 | 4.038 | −17.119 | 70.287 | 1.00 | 32.53 |
| ATOM | 20689 | OH2 | WAT | 3759 | −8.558 | −23.729 | 1.645 | 1.00 | 40.40 |
| ATOM | 20690 | OH2 | WAT | 3760 | −9.873 | 16.727 | 21.573 | 1.00 | 27.26 |
| ATOM | 20691 | OH2 | WAT | 3761 | 35.205 | −25.918 | 42.503 | 1.00 | 30.87 |
| ATOM | 20692 | OH2 | WAT | 3762 | 1.971 | 24.125 | 46.097 | 1.00 | 25.29 |
| ATOM | 20693 | OH2 | WAT | 3763 | 53.325 | −5.919 | 60.231 | 1.00 | 28.08 |
| ATOM | 20694 | OH2 | WAT | 3764 | 10.053 | 18.498 | 6.878 | 1.00 | 33.94 |
| ATOM | 20695 | OH2 | WAT | 3765 | 19.406 | −29.642 | 19.266 | 1.00 | 28.80 |
| ATOM | 20696 | OH2 | WAT | 3766 | 16.098 | 14.581 | 21.816 | 1.00 | 36.36 |
| ATOM | 20697 | OH2 | WAT | 3767 | −6.338 | 25.111 | −7.103 | 1.00 | 28.61 |
| ATOM | 20698 | OH2 | WAT | 3768 | 48.102 | 21.342 | 3.587 | 1.00 | 36.19 |
| ATOM | 20699 | OH2 | WAT | 3769 | −9.125 | −25.882 | 48.916 | 1.00 | 39.40 |
| ATOM | 20700 | OH2 | WAT | 3770 | 6.373 | 14.161 | 30.878 | 1.00 | 25.95 |
| ATOM | 20701 | OH2 | WAT | 3771 | 18.098 | 37.562 | 30.318 | 1.00 | 29.01 |
| ATOM | 20702 | OH2 | WAT | 3772 | −4.272 | −41.366 | 30.616 | 1.00 | 33.77 |
| ATOM | 20703 | OH2 | WAT | 3773 | −24.088 | −15.889 | 26.441 | 1.00 | 33.05 |
| ATOM | 20704 | OH2 | WAT | 3774 | 5.765 | 13.265 | 64.636 | 1.00 | 43.14 |
| ATOM | 20705 | OH2 | WAT | 3775 | −24.832 | −2.210 | 12.951 | 1.00 | 32.20 |
| ATOM | 20706 | OH2 | WAT | 3776 | 35.826 | −23.014 | 22.538 | 1.00 | 27.58 |
| ATOM | 20707 | OH2 | WAT | 3777 | 34.896 | −31.558 | 17.395 | 1.00 | 35.22 |
| ATOM | 20708 | OH2 | WAT | 3778 | 12.653 | 27.171 | 37.327 | 1.00 | 34.77 |
| ATOM | 20709 | OH2 | WAT | 3779 | 26.085 | 8.717 | 7.225 | 1.00 | 27.52 |
| ATOM | 20710 | OH2 | WAT | 3780 | 14.808 | 13.935 | 2.743 | 1.00 | 28.55 |
| ATOM | 20711 | OH2 | WAT | 3781 | 7.008 | 41.307 | 8.799 | 1.00 | 21.88 |
| ATOM | 20712 | OH2 | WAT | 3782 | −29.750 | −15.615 | 50.483 | 1.00 | 33.58 |
| ATOM | 20713 | OH2 | WAT | 3783 | 42.042 | 7.501 | 10.339 | 1.00 | 32.40 |
| ATOM | 20714 | OH2 | WAT | 3784 | 38.647 | −37.049 | 26.470 | 1.00 | 27.62 |
| ATOM | 20715 | OH2 | WAT | 3785 | 20.114 | −10.501 | 38.007 | 1.00 | 33.84 |
| ATOM | 20716 | OH2 | WAT | 3786 | 33.193 | 13.567 | 2.822 | 1.00 | 33.07 |
| ATOM | 20717 | OH2 | WAT | 3787 | 41.018 | −5.644 | 6.389 | 1.00 | 32.27 |
| ATOM | 20718 | OH2 | WAT | 3788 | 1.927 | 13.217 | 17.887 | 1.00 | 23.25 |
| ATOM | 20719 | OH2 | WAT | 3789 | 0.063 | −39.198 | 5.123 | 1.00 | 40.67 |
| ATOM | 20720 | OH2 | WAT | 3790 | 14.610 | −2.645 | 49.001 | 1.00 | 23.41 |
| ATOM | 20721 | OH2 | WAT | 3791 | 29.017 | −41.765 | 34.853 | 1.00 | 29.00 |
| ATOM | 20722 | OH2 | WAT | 3792 | 16.478 | 28.346 | 37.634 | 1.00 | 26.47 |
| ATOM | 20723 | OH2 | WAT | 3793 | 2.863 | −52.758 | 24.049 | 1.00 | 35.61 |
| ATOM | 20724 | OH2 | WAT | 3794 | −11.416 | 19.596 | 17.671 | 1.00 | 27.30 |
| ATOM | 20725 | OH2 | WAT | 3795 | 32.706 | −47.734 | 14.100 | 1.00 | 27.60 |
| ATOM | 20726 | OH2 | WAT | 3796 | 3.656 | 6.892 | 17.830 | 1.00 | 29.51 |
| ATOM | 20727 | OH2 | WAT | 3797 | 34.924 | −21.245 | −11.957 | 1.00 | 26.01 |
| ATOM | 20728 | OH2 | WAT | 3798 | −23.350 | 8.284 | 62.170 | 1.00 | 26.06 |
| ATOM | 20729 | OH2 | WAT | 3799 | 21.618 | 5.515 | 19.392 | 1.00 | 30.34 |
| ATOM | 20730 | OH2 | WAT | 3800 | 3.296 | 31.352 | 59.084 | 1.00 | 20.90 |
| ATOM | 20731 | OH2 | WAT | 3801 | 11.215 | −4.478 | 10.504 | 1.00 | 26.27 |
| ATOM | 20732 | OH2 | WAT | 3802 | 17.465 | 28.856 | 49.985 | 1.00 | 32.00 |
| ATOM | 20733 | OH2 | WAT | 3803 | 30.546 | 21.709 | 36.983 | 1.00 | 33.86 |
| ATOM | 20734 | OH2 | WAT | 3804 | −13.822 | 6.385 | 18.289 | 1.00 | 26.93 |
| ATOM | 20735 | OH2 | WAT | 3805 | 40.370 | 26.108 | 57.308 | 1.00 | 33.23 |
| ATOM | 20736 | OH2 | WAT | 3806 | −18.730 | 19.657 | 0.767 | 1.00 | 34.40 |
| ATOM | 20737 | OH2 | WAT | 3807 | 46.787 | −11.310 | 5.258 | 1.00 | 33.73 |
| ATOM | 20738 | OH2 | WAT | 3808 | 6.196 | 37.401 | 65.886 | 1.00 | 25.32 |
| ATOM | 20739 | OH2 | WAT | 3809 | 33.898 | −16.337 | 34.979 | 1.00 | 25.93 |
| ATOM | 20740 | OH2 | WAT | 3810 | −29.017 | −32.892 | 18.794 | 1.00 | 34.70 |
| ATOM | 20741 | OH2 | WAT | 3811 | 5.143 | 46.820 | 14.606 | 1.00 | 30.65 |
| ATOM | 20742 | OH2 | WAT | 3812 | 7.988 | −3.484 | 64.242 | 1.00 | 31.97 |
| ATOM | 20743 | OH2 | WAT | 3813 | 5.432 | −9.598 | 10.054 | 1.00 | 29.06 |
| ATOM | 20744 | OH2 | WAT | 3814 | 19.243 | 6.256 | −9.064 | 1.00 | 30.91 |
| ATOM | 20745 | OH2 | WAT | 3815 | 43.635 | −21.259 | 49.737 | 1.00 | 28.64 |
| ATOM | 20746 | OH2 | WAT | 3816 | −5.313 | 6.920 | 37.151 | 1.00 | 30.93 |
| ATOM | 20747 | OH2 | WAT | 3817 | 4.504 | 24.582 | −5.136 | 1.00 | 32.05 |
| ATOM | 20748 | OH2 | WAT | 3818 | 10.520 | 3.740 | −26.086 | 1.00 | 34.31 |
| ATOM | 20749 | OH2 | WAT | 3819 | 62.545 | 14.377 | 25.314 | 1.00 | 43.40 |
| ATOM | 20750 | OH2 | WAT | 3820 | 9.862 | −15.964 | 13.553 | 1.00 | 29.65 |
| ATOM | 20751 | OH2 | WAT | 3821 | −0.387 | −50.398 | 19.754 | 1.00 | 33.87 |
| ATOM | 20752 | OH2 | WAT | 3822 | 2.304 | −6.547 | 19.517 | 1.00 | 31.26 |
| ATOM | 20753 | OH2 | WAT | 3823 | 41.519 | −23.035 | −7.279 | 1.00 | 42.38 |
| ATOM | 20754 | OH2 | WAT | 3824 | 16.162 | −20.007 | 75.071 | 1.00 | 26.45 |
| ATOM | 20755 | OH2 | WAT | 3825 | 10.546 | −21.372 | 6.968 | 1.00 | 26.45 |
| ATOM | 20756 | OH2 | WAT | 3826 | −11.694 | −23.711 | 34.633 | 1.00 | 29.90 |
| ATOM | 20757 | OH2 | WAT | 3827 | 14.382 | −28.322 | 57.340 | 1.00 | 41.11 |
| ATOM | 20758 | OH2 | WAT | 3828 | 33.815 | −17.624 | 23.792 | 1.00 | 26.84 |
| ATOM | 20759 | OH2 | WAT | 3829 | −10.831 | −0.831 | 4.970 | 1.00 | 24.36 |
| ATOM | 20760 | OH2 | WAT | 3830 | 7.070 | 13.238 | −20.561 | 1.00 | 30.58 |
| ATOM | 20761 | OH2 | WAT | 3831 | −1.423 | −43.194 | 35.158 | 1.00 | 28.74 |
| ATOM | 20762 | OH2 | WAT | 3832 | 16.519 | −33.636 | 38.322 | 1.00 | 24.42 |
| ATOM | 20763 | OH2 | WAT | 3833 | 17.520 | 22.887 | 73.186 | 1.00 | 33.05 |
| ATOM | 20764 | OH2 | WAT | 3834 | −2.872 | −6.802 | 25.146 | 1.00 | 32.72 |
| ATOM | 20765 | OH2 | WAT | 3835 | 18.475 | −54.085 | 15.078 | 1.00 | 34.15 |
| ATOM | 20766 | OH2 | WAT | 3836 | 41.800 | −20.963 | 55.130 | 1.00 | 27.59 |
| ATOM | 20767 | OH2 | WAT | 3837 | −7.482 | −4.152 | 30.462 | 1.00 | 29.41 |
| ATOM | 20768 | OH2 | WAT | 3838 | −17.203 | −14.842 | 28.926 | 1.00 | 26.31 |
| ATOM | 20769 | OH2 | WAT | 3839 | 23.941 | 17.177 | 27.224 | 1.00 | 23.52 |
| ATOM | 20770 | OH2 | WAT | 3840 | −17.547 | 18.133 | −10.035 | 1.00 | 26.05 |
| ATOM | 20771 | OH2 | WAT | 3841 | −11.739 | 21.608 | 19.442 | 1.00 | 29.94 |
| ATOM | 20772 | OH2 | WAT | 3842 | −1.775 | −31.809 | 56.054 | 1.00 | 29.15 |
| ATOM | 20773 | OH2 | WAT | 3843 | 26.925 | −28.472 | 40.778 | 1.00 | 31.95 |
| ATOM | 20774 | OH2 | WAT | 3844 | 13.649 | −18.971 | 8.359 | 1.00 | 28.20 |
| ATOM | 20775 | OH2 | WAT | 3845 | −1.364 | 38.574 | 55.862 | 1.00 | 34.22 |
| ATOM | 20776 | OH2 | WAT | 3846 | 49.423 | 21.080 | 55.366 | 1.00 | 24.86 |
| ATOM | 20777 | OH2 | WAT | 3847 | −0.268 | −12.614 | 7.599 | 1.00 | 38.86 |
| ATOM | 20778 | OH2 | WAT | 3848 | −4.305 | −53.041 | 14.218 | 1.00 | 31.46 |
| ATOM | 20779 | OH2 | WAT | 3849 | 12.067 | −31.885 | 21.029 | 1.00 | 27.96 |
| ATOM | 20780 | OH2 | WAT | 3850 | 1.046 | −35.638 | 34.940 | 1.00 | 25.32 |
| ATOM | 20781 | OH2 | WAT | 3851 | −0.470 | 22.594 | 36.668 | 1.00 | 25.41 |
| ATOM | 20782 | OH2 | WAT | 3852 | −1.223 | −1.667 | 38.153 | 1.00 | 24.06 |
| ATOM | 20783 | OH2 | WAT | 3853 | 24.503 | −1.990 | 58.124 | 1.00 | 28.69 |
| ATOM | 20784 | OH2 | WAT | 3854 | 17.864 | −13.588 | 29.400 | 1.00 | 23.88 |
| ATOM | 20785 | OH2 | WAT | 3855 | −28.291 | −14.815 | 57.051 | 1.00 | 26.82 |
| ATOM | 20786 | OH2 | WAT | 3856 | 20.622 | −5.682 | 38.418 | 1.00 | 33.21 |
| ATOM | 20787 | OH2 | WAT | 3857 | −7.378 | −24.739 | 4.250 | 1.00 | 36.23 |
| ATOM | 20788 | OH2 | WAT | 3858 | 50.437 | −6.107 | 52.678 | 1.00 | 25.23 |
| ATOM | 20789 | OH2 | WAT | 3859 | −32.230 | −8.257 | 14.449 | 1.00 | 36.13 |
| ATOM | 20790 | OH2 | WAT | 3860 | 24.061 | −50.798 | 39.085 | 1.00 | 41.51 |
| ATOM | 20791 | OH2 | WAT | 3861 | 29.292 | 7.952 | 26.061 | 1.00 | 36.29 |
| ATOM | 20792 | OH2 | WAT | 3862 | 37.184 | 10.315 | 28.928 | 1.00 | 28.67 |
| ATOM | 20793 | OH2 | WAT | 3863 | 36.355 | −20.183 | 38.839 | 1.00 | 25.50 |
| ATOM | 20794 | OH2 | WAT | 3864 | −0.607 | 5.406 | 54.644 | 1.00 | 23.50 |
| ATOM | 20795 | OH2 | WAT | 3865 | 11.302 | −47.890 | 27.551 | 1.00 | 20.57 |
| ATOM | 20796 | OH2 | WAT | 3866 | −15.007 | 23.732 | 62.688 | 1.00 | 31.11 |
| ATOM | 20797 | OH2 | WAT | 3867 | 42.181 | −21.336 | 46.400 | 1.00 | 28.39 |
| ATOM | 20798 | OH2 | WAT | 3868 | 32.816 | 16.299 | 26.091 | 1.00 | 28.85 |
| ATOM | 20799 | OH2 | WAT | 3869 | 15.315 | 17.295 | 25.040 | 1.00 | 20.18 |
| ATOM | 20800 | OH2 | WAT | 3870 | 7.289 | −17.152 | −3.029 | 1.00 | 29.44 |
| ATOM | 20801 | OH2 | WAT | 3871 | 2.717 | 42.008 | 66.372 | 1.00 | 27.89 |
| ATOM | 20802 | OH2 | WAT | 3872 | 34.914 | −28.691 | 18.175 | 1.00 | 19.17 |
| ATOM | 20803 | OH2 | WAT | 3873 | 18.616 | 13.919 | 13.090 | 1.00 | 32.78 |
| ATOM | 20804 | OH2 | WAT | 3874 | −18.466 | −37.393 | 25.244 | 1.00 | 32.88 |
| ATOM | 20805 | OH2 | WAT | 3875 | 12.836 | 43.517 | 24.922 | 1.00 | 31.86 |
| ATOM | 20806 | OH2 | WAT | 3876 | −25.602 | 12.369 | 0.165 | 1.00 | 27.51 |
| ATOM | 20807 | OH2 | WAT | 3877 | 24.200 | 7.550 | 21.778 | 1.00 | 31.56 |
| ATOM | 20808 | OH2 | WAT | 3878 | −0.145 | −8.461 | 22.519 | 1.00 | 26.05 |
| ATOM | 20809 | OH2 | WAT | 3879 | −23.514 | −16.029 | 43.033 | 1.00 | 33.79 |
| ATOM | 20810 | OH2 | WAT | 3880 | 9.948 | 50.191 | −0.282 | 1.00 | 30.98 |
| ATOM | 20811 | OH2 | WAT | 3881 | 2.703 | −13.370 | 5.739 | 1.00 | 35.46 |
| ATOM | 20812 | OH2 | WAT | 3882 | 20.914 | 25.963 | 17.772 | 1.00 | 28.78 |
| ATOM | 20813 | OH2 | WAT | 3883 | 34.528 | 11.261 | 3.220 | 1.00 | 31.17 |
| ATOM | 20814 | OH2 | WAT | 3884 | 16.134 | −53.497 | 28.465 | 1.00 | 33.54 |
| ATOM | 20815 | OH2 | WAT | 3885 | −2.387 | 38.437 | 61.965 | 1.00 | 31.00 |
| ATOM | 20816 | OH2 | WAT | 3886 | 43.549 | −6.595 | 8.700 | 1.00 | 33.92 |
| ATOM | 20817 | OH2 | WAT | 3887 | 35.170 | −2.113 | 5.111 | 1.00 | 23.74 |
| ATOM | 20818 | OH2 | WAT | 3888 | −25.332 | −27.546 | 33.584 | 1.00 | 32.68 |
| ATOM | 20819 | OH2 | WAT | 3889 | 9.100 | 5.403 | 58.911 | 1.00 | 28.07 |
| ATOM | 20820 | OH2 | WAT | 3890 | −2.005 | 3.998 | 40.706 | 1.00 | 30.75 |
| ATOM | 20821 | OH2 | WAT | 3891 | −7.292 | −27.296 | 1.998 | 1.00 | 38.01 |
| ATOM | 20822 | OH2 | WAT | 3892 | 19.157 | −5.927 | 15.506 | 1.00 | 31.49 |
| ATOM | 20823 | OH2 | WAT | 3893 | −18.372 | 23.651 | 43.344 | 1.00 | 37.66 |
| ATOM | 20824 | OH2 | WAT | 3894 | 31.262 | −51.492 | 16.861 | 1.00 | 24.13 |
| ATOM | 20825 | OH2 | WAT | 3895 | −26.499 | 9.900 | 3.726 | 1.00 | 39.65 |
| ATOM | 20826 | OH2 | WAT | 3896 | 4.097 | 31.182 | 75.075 | 1.00 | 32.65 |
| ATOM | 20827 | OH2 | WAT | 3897 | 2.547 | 4.374 | 39.181 | 1.00 | 30.86 |
| ATOM | 20828 | OH2 | WAT | 3898 | 15.775 | −19.780 | −5.586 | 1.00 | 40.14 |
| ATOM | 20829 | OH2 | WAT | 3899 | 25.581 | −38.670 | 36.519 | 1.00 | 28.00 |
| ATOM | 20830 | OH2 | WAT | 3900 | −5.538 | −11.112 | 0.952 | 1.00 | 35.04 |
| ATOM | 20831 | OH2 | WAT | 3901 | 22.835 | −4.612 | 16.494 | 1.00 | 29.12 |
| ATOM | 20832 | OH2 | WAT | 3902 | 12.797 | 17.517 | 28.765 | 1.00 | 25.18 |
| ATOM | 20833 | OH2 | WAT | 3903 | 4.058 | −26.546 | 4.576 | 1.00 | 30.15 |
| ATOM | 20834 | OH2 | WAT | 3904 | 29.516 | −1.506 | 18.412 | 1.00 | 35.13 |
| ATOM | 20835 | OH2 | WAT | 3905 | 8.641 | −41.868 | 14.364 | 1.00 | 24.18 |
| ATOM | 20836 | OH2 | WAT | 3906 | 17.066 | 16.097 | 14.366 | 1.00 | 23.39 |
| ATOM | 20837 | OH2 | WAT | 3907 | 24.988 | −0.851 | −20.057 | 1.00 | 29.57 |
| ATOM | 20838 | OH2 | WAT | 3908 | −5.479 | −33.973 | 28.883 | 1.00 | 27.02 |
| ATOM | 20839 | OH2 | WAT | 3909 | −35.530 | −14.033 | 12.897 | 1.00 | 34.50 |
| ATOM | 20840 | OH2 | WAT | 3910 | −19.863 | 23.952 | 46.624 | 1.00 | 32.52 |
| ATOM | 20841 | OH2 | WAT | 3911 | 11.250 | −32.899 | 13.772 | 1.00 | 26.39 |
| ATOM | 20842 | OH2 | WAT | 3912 | 18.533 | 26.176 | 31.373 | 1.00 | 26.30 |
| ATOM | 20843 | OH2 | WAT | 3913 | 11.461 | −44.385 | 34.329 | 1.00 | 36.66 |
| ATOM | 20844 | OH2 | WAT | 3914 | 49.046 | 8.099 | 27.030 | 1.00 | 31.36 |
| ATOM | 20845 | OH2 | WAT | 3915 | −6.962 | 47.797 | 20.546 | 1.00 | 35.11 |
| ATOM | 20846 | OH2 | WAT | 3916 | −11.454 | 27.697 | 39.038 | 1.00 | 34.68 |
| ATOM | 20847 | OH2 | WAT | 3917 | −17.534 | −6.063 | −15.347 | 1.00 | 33.23 |
| ATOM | 20848 | OH2 | WAT | 3918 | −20.662 | 24.797 | 51.285 | 1.00 | 38.11 |
| ATOM | 20849 | OH2 | WAT | 3919 | −15.182 | 22.940 | −8.359 | 1.00 | 32.06 |
| ATOM | 20850 | OH2 | WAT | 3920 | −5.318 | 20.153 | −21.024 | 1.00 | 36.60 |
| ATOM | 20851 | OH2 | WAT | 3921 | 29.287 | 32.027 | 23.219 | 1.00 | 29.21 |
| ATOM | 20852 | OH2 | WAT | 3922 | 5.296 | 4.996 | 60.230 | 1.00 | 27.47 |
| ATOM | 20853 | OH2 | WAT | 3923 | 0.917 | −45.819 | 34.364 | 1.00 | 33.20 |
| ATOM | 20854 | OH2 | WAT | 3924 | 3.488 | 6.382 | 55.858 | 1.00 | 25.52 |
| ATOM | 20855 | OH2 | WAT | 3925 | 32.958 | 6.677 | 47.699 | 1.00 | 22.12 |
| ATOM | 20856 | OH2 | WAT | 3926 | −23.321 | −11.254 | 27.790 | 1.00 | 35.27 |
| ATOM | 20857 | OH2 | WAT | 3927 | 32.860 | 27.703 | 46.551 | 1.00 | 32.44 |
| ATOM | 20858 | OH2 | WAT | 3928 | −1.426 | 25.054 | 11.614 | 1.00 | 29.69 |
| ATOM | 20859 | OH2 | WAT | 3929 | −15.519 | 16.582 | 14.184 | 1.00 | 28.11 |
| ATOM | 20860 | OH2 | WAT | 3930 | 43.595 | −17.787 | 8.451 | 1.00 | 30.25 |
| ATOM | 20861 | OH2 | WAT | 3931 | 23.063 | 6.691 | 36.902 | 1.00 | 33.20 |
| ATOM | 20862 | OH2 | WAT | 3932 | −9.032 | 1.177 | 27.078 | 1.00 | 34.25 |
| ATOM | 20863 | OH2 | WAT | 3933 | 31.257 | 21.599 | 1.621 | 1.00 | 37.72 |
| ATOM | 20864 | OH2 | WAT | 3934 | 38.507 | −21.072 | −10.854 | 1.00 | 25.61 |
| ATOM | 20865 | OH2 | WAT | 3935 | −23.025 | 12.738 | −10.126 | 1.00 | 28.93 |
| ATOM | 20866 | OH2 | WAT | 3936 | 22.699 | −11.887 | 36.490 | 1.00 | 24.74 |
| ATOM | 20867 | OH2 | WAT | 3937 | 17.275 | −3.978 | 40.410 | 1.00 | 32.51 |
| ATOM | 20868 | OH2 | WAT | 3938 | 7.861 | 15.365 | 34.973 | 1.00 | 44.23 |
| ATOM | 20869 | OH2 | WAT | 3939 | 11.275 | 25.692 | 34.511 | 1.00 | 32.64 |
| ATOM | 20870 | OH2 | WAT | 3940 | −3.313 | −38.226 | 29.667 | 1.00 | 33.46 |
| ATOM | 20871 | OH2 | WAT | 3941 | −7.692 | −42.437 | 29.109 | 1.00 | 43.80 |
| ATOM | 20872 | OH2 | WAT | 3942 | −13.863 | 31.441 | 25.663 | 1.00 | 37.38 |
| ATOM | 20873 | OH2 | WAT | 3943 | −12.474 | −21.794 | 42.793 | 1.00 | 34.53 |
| ATOM | 20874 | OH2 | WAT | 3944 | −3.638 | 41.102 | 62.525 | 1.00 | 37.56 |
| ATOM | 20875 | OH2 | WAT | 3945 | −18.227 | 25.816 | 55.937 | 1.00 | 30.59 |
| ATOM | 20876 | OH2 | WAT | 3946 | −15.985 | −38.554 | 28.518 | 1.00 | 32.77 |
| ATOM | 20877 | OH2 | WAT | 3947 | 34.669 | −24.614 | 67.209 | 1.00 | 25.76 |
| ATOM | 20878 | OH2 | WAT | 3948 | 54.577 | 15.128 | 52.543 | 1.00 | 33.52 |
| ATOM | 20879 | OH2 | WAT | 3949 | 0.887 | −14.120 | 4.073 | 1.00 | 36.74 |
| ATOM | 20880 | OH2 | WAT | 3950 | −15.097 | 35.053 | 18.039 | 1.00 | 37.02 |
| ATOM | 20881 | OH2 | WAT | 3951 | 15.818 | 2.198 | 9.301 | 1.00 | 29.29 |
| ATOM | 20882 | OH2 | WAT | 3952 | −22.631 | 7.920 | −14.709 | 1.00 | 23.92 |
| ATOM | 20883 | OH2 | WAT | 3953 | 1.132 | −17.635 | 32.905 | 1.00 | 29.71 |
| ATOM | 20884 | OH2 | WAT | 3954 | −24.033 | −11.665 | 68.949 | 1.00 | 36.66 |
| ATOM | 20885 | OH2 | WAT | 3955 | 31.947 | −26.774 | 44.122 | 1.00 | 27.95 |
| ATOM | 20886 | OH2 | WAT | 3956 | 27.559 | −23.512 | 73.698 | 1.00 | 34.23 |
| ATOM | 20887 | OH2 | WAT | 3957 | 40.516 | 20.972 | 40.033 | 1.00 | 37.10 |
| ATOM | 20888 | OH2 | WAT | 3958 | −21.789 | −38.079 | 25.502 | 1.00 | 23.71 |
| ATOM | 20889 | OH2 | WAT | 3959 | 27.138 | 35.825 | 22.971 | 1.00 | 29.72 |
| ATOM | 20890 | OH2 | WAT | 3960 | 6.627 | 13.854 | 67.881 | 1.00 | 33.43 |
| ATOM | 20891 | OH2 | WAT | 3961 | −7.495 | 30.319 | 65.877 | 1.00 | 47.88 |
| ATOM | 20892 | OH2 | WAT | 3962 | 39.945 | −16.981 | 59.063 | 1.00 | 41.80 |
| ATOM | 20893 | OH2 | WAT | 3963 | 10.451 | −0.990 | 11.902 | 1.00 | 42.44 |
| ATOM | 20894 | OH2 | WAT | 3964 | −19.187 | 17.170 | 7.811 | 1.00 | 34.07 |
| ATOM | 20895 | OH2 | WAT | 3965 | 55.677 | 8.381 | 57.068 | 1.00 | 34.33 |
| ATOM | 20896 | OH2 | WAT | 3966 | −21.554 | 13.101 | 7.475 | 1.00 | 24.12 |
| ATOM | 20897 | OH2 | WAT | 3967 | 8.150 | −27.034 | 50.434 | 1.00 | 39.84 |
| ATOM | 20898 | OH2 | WAT | 3968 | −18.831 | 9.121 | 46.959 | 1.00 | 30.55 |
| ATOM | 20899 | OH2 | WAT | 3969 | 50.680 | 1.961 | 15.800 | 1.00 | 35.02 |
| ATOM | 20900 | OH2 | WAT | 3970 | 35.217 | −49.138 | 25.289 | 1.00 | 34.87 |
| ATOM | 20901 | OH2 | WAT | 3971 | −29.398 | −8.042 | 11.100 | 1.00 | 31.40 |
| ATOM | 20902 | OH2 | WAT | 3972 | 19.447 | 17.441 | 13.441 | 1.00 | 29.33 |
| ATOM | 20903 | OH2 | WAT | 3973 | −13.103 | 5.510 | 51.486 | 1.00 | 32.37 |
| ATOM | 20904 | OH2 | WAT | 3974 | 35.572 | −38.920 | 31.797 | 1.00 | 29.21 |
| ATOM | 20905 | OH2 | WAT | 3975 | 42.490 | −6.432 | 48.177 | 1.00 | 37.33 |
| ATOM | 20906 | OH2 | WAT | 3976 | 55.908 | 7.452 | 19.225 | 1.00 | 41.37 |
| ATOM | 20907 | OH2 | WAT | 3977 | −23.545 | 12.346 | −12.731 | 1.00 | 36.65 |
| ATOM | 20908 | OH2 | WAT | 3978 | 29.194 | −25.699 | 42.423 | 1.00 | 28.23 |
| ATOM | 20909 | OH2 | WAT | 3979 | 24.546 | 4.768 | 33.624 | 1.00 | 34.00 |
| ATOM | 20910 | OH2 | WAT | 3980 | 11.846 | −56.638 | 27.547 | 1.00 | 36.56 |
| ATOM | 20911 | OH2 | WAT | 3981 | −11.970 | −28.236 | 68.101 | 1.00 | 31.56 |
| ATOM | 20912 | OH2 | WAT | 3982 | 34.680 | 0.521 | 51.130 | 1.00 | 26.18 |
| ATOM | 20913 | OH2 | WAT | 3983 | 24.960 | −27.638 | 15.617 | 1.00 | 35.13 |
| ATOM | 20914 | OH2 | WAT | 3984 | 12.178 | −47.174 | 31.370 | 1.00 | 34.11 |
| ATOM | 20915 | OH2 | WAT | 3985 | −12.298 | −29.408 | 25.980 | 1.00 | 20.06 |
| ATOM | 20916 | OH2 | WAT | 3986 | −5.239 | −4.738 | 26.410 | 1.00 | 30.64 |
| ATOM | 20917 | OH2 | WAT | 3987 | 7.222 | 25.190 | 72.525 | 1.00 | 29.80 |
| ATOM | 20918 | OH2 | WAT | 3988 | −8.316 | −17.085 | 4.491 | 1.00 | 32.12 |
| ATOM | 20919 | OH2 | WAT | 3989 | 14.875 | −37.478 | 62.713 | 1.00 | 36.51 |
| ATOM | 20920 | OH2 | WAT | 3990 | −0.318 | −32.480 | 35.647 | 1.00 | 31.85 |
| ATOM | 20921 | OH2 | WAT | 3991 | 26.991 | 6.700 | 24.647 | 1.00 | 28.93 |
| ATOM | 20922 | OH2 | WAT | 3992 | 9.431 | −16.988 | 4.689 | 1.00 | 31.20 |
| ATOM | 20923 | OH2 | WAT | 3993 | 23.751 | 35.159 | 59.828 | 1.00 | 30.73 |
| ATOM | 20924 | OH2 | WAT | 3994 | −29.524 | 4.920 | 48.463 | 1.00 | 38.77 |
| ATOM | 20925 | OH2 | WAT | 3995 | −10.036 | 20.469 | 3.697 | 1.00 | 27.33 |
| ATOM | 20926 | OH2 | WAT | 3996 | 13.430 | −13.926 | 30.486 | 1.00 | 27.55 |
| ATOM | 20927 | OH2 | WAT | 3997 | −19.648 | −30.155 | 4.910 | 1.00 | 27.29 |
| ATOM | 20928 | OH2 | WAT | 3998 | −1.861 | 2.934 | 16.906 | 1.00 | 22.81 |
| ATOM | 20929 | OH2 | WAT | 3999 | −6.312 | −20.345 | 39.194 | 1.00 | 36.98 |
| ATOM | 20930 | OH2 | WAT | 4000 | 0.162 | 16.385 | 31.968 | 1.00 | 40.85 |
| ATOM | 20931 | OH2 | WAT | 4001 | 37.331 | −42.808 | 29.079 | 1.00 | 35.13 |
| ATOM | 20932 | OH2 | WAT | 4002 | 1.728 | 0.651 | 41.556 | 1.00 | 40.93 |
| ATOM | 20933 | OH2 | WAT | 4003 | 7.955 | −18.489 | −7.241 | 1.00 | 41.83 |
| ATOM | 20934 | OH2 | WAT | 4004 | −2.324 | −24.753 | 41.326 | 1.00 | 36.27 |
| ATOM | 20935 | OH2 | WAT | 4005 | −14.001 | −19.794 | −0.066 | 1.00 | 34.42 |
| ATOM | 20936 | OH2 | WAT | 4006 | 19.662 | −17.365 | −18.983 | 1.00 | 32.80 |
| ATOM | 20937 | OH2 | WAT | 4007 | 10.564 | −14.743 | −13.275 | 1.00 | 29.84 |
| ATOM | 20938 | OH2 | WAT | 4008 | 15.113 | 15.031 | 39.754 | 1.00 | 30.34 |
| ATOM | 20939 | OH2 | WAT | 4009 | 35.331 | −21.374 | 20.527 | 1.00 | 31.28 |
| ATOM | 20940 | OH2 | WAT | 4010 | 22.819 | 15.879 | 3.191 | 1.00 | 35.03 |
| ATOM | 20941 | OH2 | WAT | 4011 | −10.543 | −46.147 | 9.362 | 1.00 | 38.45 |
| ATOM | 20942 | OH2 | WAT | 4012 | −16.594 | 34.561 | 56.964 | 1.00 | 28.81 |
| ATOM | 20943 | OH2 | WAT | 4013 | −10.146 | 32.295 | 63.758 | 1.00 | 37.29 |
| ATOM | 20944 | OH2 | WAT | 4014 | 48.882 | −7.382 | 51.071 | 1.00 | 37.37 |
| ATOM | 20945 | OH2 | WAT | 4015 | 29.920 | 7.293 | 29.541 | 1.00 | 33.85 |
| ATOM | 20946 | OH2 | WAT | 4016 | 19.625 | −12.921 | 72.073 | 1.00 | 36.63 |
| ATOM | 20947 | OH2 | WAT | 4017 | 13.368 | −34.511 | 37.285 | 1.00 | 29.54 |
| ATOM | 20948 | OH2 | WAT | 4018 | −5.762 | −7.177 | 29.837 | 1.00 | 31.23 |
| ATOM | 20949 | OH2 | WAT | 4019 | 11.378 | −38.956 | 13.198 | 1.00 | 31.14 |
| ATOM | 20950 | OH2 | WAT | 4020 | −7.090 | −14.815 | 72.040 | 1.00 | 26.77 |
| ATOM | 20951 | OH2 | WAT | 4021 | 2.252 | 22.804 | −7.049 | 1.00 | 32.91 |
| ATOM | 20952 | OH2 | WAT | 4022 | −3.449 | 14.552 | −24.093 | 1.00 | 36.29 |
| ATOM | 20953 | OH2 | WAT | 4023 | −8.112 | 3.142 | −24.672 | 1.00 | 33.84 |
| ATOM | 20954 | OH2 | WAT | 4024 | 18.026 | −21.806 | 8.212 | 1.00 | 29.93 |
| ATOM | 20955 | OH2 | WAT | 4025 | 60.020 | 17.539 | 13.000 | 1.00 | 44.67 |
| ATOM | 20956 | OH2 | WAT | 4026 | −26.370 | −39.869 | 16.104 | 1.00 | 41.85 |
| ATOM | 20957 | OH2 | WAT | 4027 | 16.764 | 19.360 | 52.609 | 1.00 | 28.33 |
| ATOM | 20958 | OH2 | WAT | 4028 | 35.990 | 24.016 | 40.511 | 1.00 | 34.74 |
| ATOM | 20959 | OH2 | WAT | 4029 | 57.458 | 10.198 | 50.817 | 1.00 | 32.75 |
| ATOM | 20960 | OH2 | WAT | 4030 | 1.804 | −47.829 | 30.519 | 1.00 | 26.50 |
| ATOM | 20961 | OH2 | WAT | 4031 | −10.626 | −9.725 | 72.521 | 1.00 | 38.53 |
| ATOM | 20962 | OH2 | WAT | 4032 | 43.991 | −27.164 | 51.358 | 1.00 | 25.47 |
| ATOM | 20963 | OH2 | WAT | 4033 | −28.328 | −39.519 | 17.822 | 1.00 | 37.78 |
| ATOM | 20964 | OH2 | WAT | 4034 | −29.987 | −22.766 | 8.334 | 1.00 | 31.96 |
| ATOM | 20965 | OH2 | WAT | 4035 | 2.256 | 13.057 | −20.781 | 1.00 | 40.03 |
| ATOM | 20966 | OH2 | WAT | 4036 | 25.989 | −20.630 | −14.613 | 1.00 | 32.18 |
| ATOM | 20967 | OH2 | WAT | 4037 | 2.215 | −18.558 | 69.068 | 1.00 | 34.21 |
| ATOM | 20968 | OH2 | WAT | 4038 | −36.511 | −18.857 | 14.921 | 1.00 | 29.01 |
| ATOM | 20969 | OH2 | WAT | 4039 | 2.072 | 25.410 | 67.667 | 1.00 | 25.96 |
| ATOM | 20970 | OH2 | WAT | 4040 | −4.117 | −40.064 | 35.025 | 1.00 | 27.36 |
| ATOM | 20971 | OH2 | WAT | 4041 | 3.196 | 26.403 | 17.608 | 1.00 | 32.65 |
| ATOM | 20972 | OH2 | WAT | 4042 | 4.213 | −11.022 | 21.040 | 1.00 | 25.61 |
| ATOM | 20973 | OH2 | WAT | 4043 | 20.672 | 6.288 | 43.154 | 1.00 | 11.39 |
| ATOM | 20974 | OH2 | WAT | 4044 | 24.230 | 13.675 | 24.481 | 1.00 | 17.87 |
| ATOM | 20975 | OH2 | WAT | 4045 | 11.491 | −12.039 | 39.730 | 1.00 | 16.52 |
| ATOM | 20976 | OH2 | WAT | 4046 | −2.958 | −45.541 | 12.487 | 1.00 | 25.04 |
| ATOM | 20977 | OH2 | WAT | 4047 | 21.124 | 0.639 | 54.913 | 1.00 | 19.00 |
| ATOM | 20978 | OH2 | WAT | 4048 | 10.830 | −15.313 | 20.740 | 1.00 | 17.94 |
| ATOM | 20979 | OH2 | WAT | 4049 | 7.082 | 3.993 | 47.118 | 1.00 | 22.59 |
| ATOM | 20980 | OH2 | WAT | 4050 | −15.003 | 20.551 | −11.225 | 1.00 | 23.46 |
| ATOM | 20981 | OH2 | WAT | 4051 | 5.614 | −3.781 | 42.911 | 1.00 | 18.68 |
| ATOM | 20982 | OH2 | WAT | 4052 | 2.878 | −5.383 | 14.276 | 1.00 | 21.86 |
| ATOM | 20983 | OH2 | WAT | 4053 | −28.975 | 0.173 | 43.415 | 1.00 | 25.48 |
| ATOM | 20984 | OH2 | WAT | 4054 | 18.990 | 13.786 | 23.082 | 1.00 | 34.23 |
| ATOM | 20985 | OH2 | WAT | 4055 | 14.863 | 15.999 | 12.872 | 1.00 | 22.69 |
| ATOM | 20986 | OH2 | WAT | 4056 | 4.404 | −26.590 | 36.835 | 1.00 | 20.51 |
| ATOM | 20987 | OH2 | WAT | 4057 | 16.881 | 17.463 | 50.738 | 1.00 | 25.65 |
| ATOM | 20988 | OH2 | WAT | 4058 | 7.759 | −15.429 | 23.245 | 1.00 | 25.72 |
| ATOM | 20989 | OH2 | WAT | 4059 | 1.948 | −8.274 | 37.874 | 1.00 | 24.67 |
| ATOM | 20990 | OH2 | WAT | 4060 | −0.846 | 5.377 | 57.566 | 1.00 | 26.92 |
| ATOM | 20991 | OH2 | WAT | 4061 | 11.838 | −16.631 | 28.299 | 1.00 | 26.60 |
| ATOM | 20992 | OH2 | WAT | 4062 | 8.670 | −7.127 | 7.982 | 1.00 | 23.89 |
| ATOM | 20993 | OH2 | WAT | 4063 | 7.943 | 4.878 | 13.325 | 1.00 | 29.72 |
| ATOM | 20994 | OH2 | WAT | 4064 | −7.873 | −18.149 | 38.159 | 1.00 | 24.67 |
| ATOM | 20995 | OH2 | WAT | 4065 | 19.946 | −16.578 | 22.471 | 1.00 | 21.68 |
| ATOM | 20996 | OH2 | WAT | 4066 | 26.755 | −23.578 | 42.730 | 1.00 | 30.96 |
| ATOM | 20997 | OH2 | WAT | 4067 | 19.813 | −21.428 | 77.332 | 1.00 | 26.96 |
| ATOM | 20998 | OH2 | WAT | 4068 | −14.611 | −27.813 | 24.596 | 1.00 | 22.85 |
| ATOM | 20999 | OH2 | WAT | 4069 | 10.556 | −42.741 | 36.183 | 1.00 | 30.39 |
| ATOM | 21000 | OH2 | WAT | 4070 | 9.656 | 11.940 | 15.495 | 1.00 | 33.39 |
| ATOM | 21001 | OH2 | WAT | 4071 | 15.586 | −6.472 | 42.294 | 1.00 | 26.75 |
| ATOM | 21002 | OH2 | WAT | 4072 | 21.817 | 15.587 | 28.120 | 1.00 | 36.02 |
| ATOM | 21003 | OH2 | WAT | 4073 | 42.955 | −29.610 | 52.935 | 1.00 | 30.64 |
| ATOM | 21004 | OH2 | WAT | 4074 | −5.163 | −46.625 | 9.621 | 1.00 | 25.65 |
| ATOM | 21005 | OH2 | WAT | 4075 | −22.661 | −39.337 | 16.988 | 1.00 | 29.68 |
| ATOM | 21006 | OH2 | WAT | 4076 | 12.558 | 54.160 | 17.405 | 1.00 | 42.52 |
| ATOM | 21007 | OH2 | WAT | 4077 | 14.558 | −21.050 | −8.217 | 1.00 | 35.25 |
| ATOM | 21008 | OH2 | WAT | 4078 | 14.627 | −21.292 | 77.322 | 1.00 | 26.15 |
| ATOM | 21009 | OH2 | WAT | 4079 | 38.511 | 12.796 | 27.817 | 1.00 | 32.17 |
| ATOM | 21010 | OH2 | WAT | 4080 | 6.634 | −11.234 | 41.030 | 1.00 | 33.51 |
| ATOM | 21011 | OH2 | WAT | 4081 | 19.800 | 4.002 | −7.345 | 1.00 | 31.84 |
| ATOM | 21012 | OH2 | WAT | 4082 | −11.712 | 14.246 | −16.297 | 1.00 | 37.20 |
| ATOM | 21013 | OH2 | WAT | 4083 | −1.181 | 12.792 | −23.740 | 1.00 | 27.77 |
| ATOM | 21014 | OH2 | WAT | 4084 | −13.478 | 31.890 | 22.898 | 1.00 | 35.63 |
| ATOM | 21015 | OH2 | WAT | 4085 | −26.720 | −9.960 | 27.431 | 1.00 | 31.59 |
| ATOM | 21016 | OH2 | WAT | 4086 | 21.413 | −27.440 | 21.028 | 1.00 | 46.92 |
| ATOM | 21017 | OH2 | WAT | 4087 | 21.955 | −19.353 | −18.425 | 1.00 | 22.71 |
| ATOM | 21018 | OH2 | WAT | 4088 | 12.468 | −6.264 | 66.043 | 1.00 | 33.46 |
| ATOM | 21019 | OH2 | WAT | 4089 | 13.249 | −36.608 | 12.813 | 1.00 | 30.40 |
| ATOM | 21020 | OH2 | WAT | 4090 | 31.960 | −4.177 | 33.906 | 1.00 | 33.61 |
| ATOM | 21021 | OH2 | WAT | 4091 | 7.583 | −54.047 | 28.356 | 1.00 | 26.52 |
| ATOM | 21022 | OH2 | WAT | 4092 | 0.614 | 21.888 | 44.748 | 1.00 | 35.94 |
| ATOM | 21023 | OH2 | WAT | 4093 | 37.538 | −5.989 | 37.641 | 1.00 | 32.64 |
| ATOM | 21024 | OH2 | WAT | 4094 | 21.638 | 5.075 | 39.740 | 1.00 | 17.05 |
| ATOM | 21025 | OH2 | WAT | 4095 | 28.273 | −46.828 | 32.920 | 1.00 | 24.39 |
| ATOM | 21026 | OH2 | WAT | 4096 | 11.835 | −19.653 | 5.133 | 1.00 | 25.49 |
| ATOM | 21027 | OH2 | WAT | 4097 | −13.207 | −44.364 | 8.973 | 1.00 | 37.09 |
| ATOM | 21028 | OH2 | WAT | 4098 | 27.647 | 11.322 | 5.334 | 1.00 | 32.90 |
| ATOM | 21029 | OH2 | WAT | 4099 | 10.259 | −49.393 | 8.411 | 1.00 | 26.63 |
| ATOM | 21030 | OH2 | WAT | 4100 | 4.110 | −39.224 | 10.206 | 1.00 | 32.32 |
| ATOM | 21031 | OH2 | WAT | 4101 | 23.743 | −40.087 | 37.856 | 1.00 | 50.14 |
| ATOM | 21032 | OH2 | WAT | 4102 | −24.342 | −7.153 | 30.038 | 1.00 | 30.58 |
| ATOM | 21033 | OH2 | WAT | 4103 | −0.619 | 11.724 | −26.102 | 1.00 | 35.65 |
| ATOM | 21034 | OH2 | WAT | 4104 | −9.425 | 29.111 | 31.719 | 1.00 | 37.15 |
| ATOM | 21035 | OH2 | WAT | 4105 | 32.807 | −16.675 | 32.368 | 1.00 | 36.40 |
| ATOM | 21036 | OH2 | WAT | 4106 | 14.318 | 14.720 | 0.095 | 1.00 | 27.76 |
| ATOM | 21037 | OH2 | WAT | 4107 | 46.641 | −18.008 | 5.068 | 1.00 | 28.99 |
| ATOM | 21038 | OH2 | WAT | 4108 | 40.504 | −35.415 | 30.077 | 1.00 | 30.68 |
| ATOM | 21039 | OH2 | WAT | 4109 | −21.465 | 1.072 | −0.752 | 1.00 | 33.32 |
| ATOM | 21040 | OH2 | WAT | 4110 | 46.498 | −3.634 | 42.684 | 1.00 | 31.74 |
| ATOM | 21041 | OH2 | WAT | 4111 | 18.777 | −56.294 | 21.233 | 1.00 | 25.38 |
| ATOM | 21042 | OH2 | WAT | 4112 | 4.896 | −54.443 | 22.412 | 1.00 | 30.18 |
| ATOM | 21043 | OH2 | WAT | 4113 | −6.278 | −12.430 | 3.805 | 1.00 | 29.79 |
| ATOM | 21044 | OH2 | WAT | 4114 | 31.595 | −33.290 | 16.444 | 1.00 | 31.75 |
| ATOM | 21045 | OH2 | WAT | 4115 | 26.923 | 26.631 | 13.935 | 1.00 | 33.99 |
| ATOM | 21046 | OH2 | WAT | 4116 | −29.043 | 5.487 | −7.898 | 1.00 | 45.43 |
| ATOM | 21047 | OH2 | WAT | 4117 | −16.588 | −19.270 | 41.769 | 1.00 | 31.95 |
| ATOM | 21048 | OH2 | WAT | 4118 | 6.070 | 9.092 | 39.715 | 1.00 | 30.14 |
| ATOM | 21049 | OH2 | WAT | 4119 | −3.516 | −31.633 | 64.053 | 1.00 | 45.65 |
| ATOM | 21050 | OH2 | WAT | 4120 | 52.126 | −3.822 | 53.340 | 1.00 | 45.42 |
| ATOM | 21051 | OH2 | WAT | 4121 | 46.817 | −4.484 | −14.025 | 1.00 | 46.99 |
| ATOM | 22052 | OH2 | WAT | 4122 | −12.592 | 3.190 | 66.991 | 1.00 | 35.76 |
| ATOM | 21053 | OH2 | WAT | 4123 | 3.350 | 29.034 | 35.859 | 1.00 | 34.34 |
| ATOM | 21054 | OH2 | WAT | 4124 | 0.606 | −33.642 | 55.873 | 1.00 | 32.91 |
| ATOM | 21055 | OH2 | WAT | 4125 | −14.891 | 36.065 | 20.896 | 1.00 | 37.79 |
| ATOM | 21056 | OH2 | WAT | 4126 | −25.092 | 9.956 | 50.529 | 1.00 | 32.04 |
| ATOM | 21057 | OH2 | WAT | 4127 | 24.645 | 3.578 | 21.558 | 1.00 | 33.84 |
| ATOM | 21058 | OH2 | WAT | 4128 | 14.966 | −15.407 | 27.047 | 1.00 | 37.48 |
| ATOM | 21059 | OH2 | WAT | 4129 | 25.311 | 0.590 | 59.424 | 1.00 | 32.26 |
| ATOM | 21060 | OH2 | WAT | 4130 | 5.921 | 2.186 | 48.638 | 1.00 | 28.21 |
| ATOM | 21061 | OH2 | WAT | 4131 | −0.194 | −33.527 | 6.693 | 1.00 | 29.78 |
| ATOM | 21062 | OH2 | WAT | 4132 | −9.038 | 35.946 | 43.790 | 1.00 | 37.55 |
| ATOM | 21063 | OH2 | WAT | 4133 | 17.435 | −31.315 | 16.469 | 1.00 | 31.72 |
| ATOM | 21064 | OH2 | WAT | 4134 | 30.359 | 5.569 | 52.711 | 1.00 | 23.92 |
| ATOM | 21065 | OH2 | WAT | 4135 | 3.683 | −12.556 | 17.583 | 1.00 | 19.93 |
| ATOM | 21066 | OH2 | WAT | 4136 | 19.428 | −23.653 | 47.102 | 1.00 | 24.33 |
| ATOM | 21067 | OH2 | WAT | 4137 | 4.278 | 27.765 | 15.197 | 1.00 | 31.50 |
| ATOM | 21068 | OH2 | WAT | 4138 | 6.578 | −15.894 | 41.477 | 1.00 | 20.38 |
| ATOM | 21069 | OH2 | WAT | 4139 | 28.301 | −2.532 | −0.522 | 1.00 | 24.30 |
| ATOM | 21070 | OH2 | WAT | 4140 | 37.632 | −34.367 | 20.394 | 1.00 | 26.22 |
| ATOM | 21071 | OH2 | WAT | 4141 | −17.982 | 10.709 | 12.655 | 1.00 | 28.44 |
| ATOM | 21072 | OH2 | WAT | 4142 | 14.342 | −13.857 | 17.205 | 1.00 | 25.59 |
| ATOM | 21073 | OH2 | WAT | 4143 | 1.448 | −15.812 | 37.733 | 1.00 | 25.27 |
| ATOM | 21074 | OH2 | WAT | 4144 | 17.870 | −19.299 | 7.566 | 1.00 | 26.09 |
| ATOM | 21075 | OH2 | WAT | 4145 | 9.956 | −49.449 | 29.174 | 1.00 | 28.96 |
| ATOM | 21076 | OH2 | WAT | 4146 | 17.480 | 3.336 | 10.956 | 1.00 | 30.39 |
| ATOM | 21077 | OH2 | WAT | 4147 | 8.517 | −4.567 | 9.940 | 1.00 | 31.22 |
| ATOM | 21078 | OH2 | WAT | 4148 | −7.175 | 0.887 | 19.891 | 1.00 | 27.12 |
| ATOM | 21079 | OH2 | WAT | 4149 | 28.989 | 41.203 | 15.263 | 1.00 | 29.63 |
| ATOM | 21080 | OH2 | WAT | 4150 | 23.580 | 3.660 | 40.283 | 1.00 | 39.46 |
| ATOM | 21081 | OH2 | WAT | 4151 | 13.879 | 13.838 | 16.264 | 1.00 | 28.18 |
| ATOM | 21082 | OH2 | WAT | 4152 | 3.363 | −53.015 | 26.505 | 1.00 | 26.41 |
| ATOM | 21083 | OH2 | WAT | 4153 | 41.742 | −31.794 | 51.912 | 1.00 | 29.88 |
| ATOM | 21084 | OH2 | WAT | 4154 | 15.462 | 21.257 | 30.971 | 1.00 | 33.63 |
| ATOM | 21085 | OH2 | WAT | 4155 | 13.429 | −11.032 | 37.773 | 1.00 | 32.69 |
| ATOM | 21086 | OH2 | WAT | 4156 | 4.765 | −2.386 | 40.740 | 1.00 | 35.83 |
| ATOM | 21087 | OH2 | WAT | 4157 | 19.023 | −19.667 | 75.203 | 1.00 | 37.72 |
| ATOM | 21088 | OH2 | WAT | 4158 | 19.034 | −11.102 | 19.527 | 1.00 | 33.73 |
| ATOM | 21089 | OH2 | WAT | 4159 | −1.263 | 16.009 | 20.936 | 1.00 | 27.24 |
| ATOM | 21090 | OH2 | WAT | 4160 | −6.256 | −45.054 | 6.939 | 1.00 | 41.51 |
| ATOM | 21091 | OH2 | WAT | 4161 | 15.761 | −28.451 | 54.807 | 1.00 | 36.09 |
| ATOM | 21092 | OH2 | WAT | 4162 | 18.439 | 14.295 | 19.227 | 1.00 | 38.49 |
| ATOM | 21093 | OH2 | WAT | 4163 | −3.972 | −20.387 | 37.814 | 1.00 | 31.16 |
| ATOM | 21094 | OH2 | WAT | 4164 | 18.013 | −1.645 | 54.687 | 1.00 | 30.47 |
| ATOM | 21095 | OH2 | WAT | 4165 | −15.633 | 33.909 | 22.604 | 1.00 | 31.22 |
| ATOM | 21096 | OH2 | WAT | 4166 | 40.780 | −8.375 | 40.145 | 1.00 | 32.51 |
| ATOM | 21097 | OH2 | WAT | 4167 | 47.802 | 30.040 | 7.228 | 1.00 | 40.79 |
| ATOM | 21098 | OH2 | WAT | 4168 | −3.032 | −32.860 | 34.864 | 1.00 | 27.66 |
| ATOM | 21099 | OH2 | WAT | 4169 | 9.198 | 20.743 | 72.320 | 1.00 | 35.07 |
| ATOM | 21100 | OH2 | WAT | 4170 | 11.975 | −21.527 | 2.995 | 1.00 | 36.62 |
| ATOM | 21101 | OH2 | WAT | 4171 | −30.445 | −6.086 | 44.089 | 1.00 | 34.70 |
| ATOM | 21102 | OH2 | WAT | 4172 | −5.261 | −11.466 | 53.111 | 1.00 | 44.39 |
| ATOM | 21103 | OH2 | WAT | 4173 | −14.399 | −20.387 | 40.817 | 1.00 | 30.20 |
| ATOM | 21104 | OH2 | WAT | 4174 | −10.618 | −14.092 | 4.622 | 1.00 | 31.64 |
| ATOM | 21105 | OH2 | WAT | 4175 | 14.591 | −19.755 | 5.914 | 1.00 | 37.61 |
| ATOM | 21106 | OH2 | WAT | 4176 | 19.353 | −22.226 | 0.817 | 1.00 | 32.55 |
| ATOM | 21107 | OH2 | WAT | 4177 | −29.776 | −5.355 | 39.885 | 1.00 | 30.32 |
| ATOM | 21108 | OH2 | WAT | 4178 | 10.534 | −13.068 | −20.889 | 1.00 | 35.70 |
| ATOM | 21109 | OH2 | WAT | 4179 | −8.062 | 5.216 | 33.169 | 1.00 | 25.98 |
| ATOM | 21110 | OH2 | WAT | 4180 | −2.522 | 30.394 | 63.742 | 1.00 | 28.47 |
| ATOM | 21111 | OH2 | WAT | 4181 | 46.009 | 3.328 | 33.070 | 1.00 | 35.13 |
| ATOM | 21112 | OH2 | WAT | 4182 | 8.360 | 8.234 | 41.757 | 1.00 | 29.18 |
| ATOM | 21113 | OH2 | WAT | 4183 | 42.814 | −18.598 | 55.686 | 1.00 | 37.99 |
| ATOM | 21114 | OH2 | WAT | 4184 | −16.001 | 22.656 | 38.987 | 1.00 | 29.36 |
| ATOM | 21115 | OH2 | WAT | 4185 | 10.259 | −36.135 | 10.535 | 1.00 | 37.88 |
| ATOM | 21116 | OH2 | WAT | 4186 | −15.684 | −9.143 | −8.092 | 1.00 | 31.91 |
| ATOM | 21117 | OH2 | WAT | 4187 | −1.090 | 1.204 | 14.459 | 1.00 | 30.11 |
| ATOM | 21118 | OH2 | WAT | 4188 | 44.289 | 2.682 | 29.166 | 1.00 | 28.69 |
| ATOM | 21119 | OH2 | WAT | 4189 | −0.552 | −28.362 | 46.000 | 1.00 | 28.10 |
| ATOM | 21120 | OH2 | WAT | 4190 | 13.135 | −5.781 | 41.932 | 1.00 | 35.53 |
| ATOM | 21121 | OH2 | WAT | 4191 | 19.734 | 30.686 | 49.844 | 1.00 | 35.70 |
| ATOM | 21122 | OH2 | WAT | 4192 | −36.378 | −21.364 | 23.565 | 1.00 | 35.60 |
| ATOM | 21123 | OH2 | WAT | 4193 | 17.251 | 0.350 | 12.314 | 1.00 | 23.98 |
| ATOM | 21124 | OH2 | WAT | 4194 | −10.271 | −38.090 | 23.438 | 1.00 | 32.22 |
| ATOM | 21125 | OH2 | WAT | 4195 | −22.687 | 7.954 | 3.699 | 1.00 | 33.28 |
| ATOM | 21126 | OH2 | WAT | 4196 | −21.951 | 15.459 | 4.933 | 1.00 | 31.65 |
| ATOM | 21127 | OH2 | WAT | 4197 | −17.403 | 21.503 | −8.388 | 1.00 | 32.40 |
| ATOM | 21128 | OH2 | WAT | 4198 | 21.977 | 9.231 | 37.108 | 1.00 | 32.87 |
| ATOM | 21129 | OH2 | WAT | 4199 | 37.419 | 17.868 | 21.884 | 1.00 | 46.31 |
| ATOM | 21130 | OH2 | WAT | 4200 | 22.196 | 9.629 | 18.773 | 1.00 | 31.77 |
| ATOM | 21131 | OH2 | WAT | 4201 | 16.692 | −23.322 | −5.245 | 1.00 | 36.91 |
| ATOM | 21132 | OH2 | WAT | 4202 | 42.777 | −33.719 | 53.939 | 1.00 | 35.86 |
| ATOM | 21133 | OH2 | WAT | 4203 | 1.354 | −2.239 | 14.757 | 1.00 | 37.10 |
| ATOM | 21134 | OH2 | WAT | 4204 | −26.952 | −3.544 | 38.440 | 1.00 | 33.73 |
| ATOM | 21135 | OH2 | WAT | 4205 | 29.239 | −5.609 | 38.042 | 1.00 | 29.97 |
| ATOM | 21136 | OH2 | WAT | 4206 | 43.138 | 0.368 | 25.089 | 1.00 | 32.24 |
| ATOM | 21137 | OH2 | WAT | 4207 | 52.415 | −7.788 | 51.945 | 1.00 | 30.80 |
| ATOM | 21138 | OH2 | WAT | 4208 | −2.220 | 0.196 | 40.301 | 1.00 | 41.13 |
| ATOM | 21139 | OH2 | WAT | 4209 | 9.166 | −36.364 | 8.279 | 1.00 | 32.28 |
| ATOM | 21140 | OH2 | WAT | 4210 | 37.549 | −23.883 | 19.085 | 1.00 | 35.85 |
| ATOM | 21141 | OH2 | WAT | 4211 | 40.366 | −43.408 | 18.178 | 1.00 | 35.18 |
| ATOM | 21142 | OH2 | WAT | 4212 | 41.259 | −39.426 | 17.720 | 1.00 | 35.03 |
| ATOM | 21143 | OH2 | WAT | 4213 | 5.966 | −16.794 | 26.485 | 1.00 | 33.99 |
| ATOM | 21144 | OH2 | WAT | 4214 | −0.504 | 24.436 | 38.709 | 1.00 | 31.34 |
| ATOM | 21145 | OH2 | WAT | 4215 | −31.816 | −7.311 | 63.265 | 1.00 | 46.67 |
| ATOM | 21146 | OH2 | WAT | 4216 | 31.602 | −27.488 | 41.591 | 1.00 | 32.87 |
| ATOM | 21147 | OH2 | WAT | 4217 | 22.491 | 8.396 | 33.548 | 1.00 | 37.01 |
| ATOM | 21148 | OH2 | WAT | 4218 | 8.588 | 14.998 | −7.928 | 1.00 | 34.59 |
| ATOM | 21149 | OH2 | WAT | 4219 | −5.071 | 39.648 | 64.146 | 1.00 | 36.17 |
| ATOM | 21150 | OH2 | WAT | 4220 | 30.649 | 7.956 | 54.006 | 1.00 | 28.56 |
| ATOM | 21151 | OH2 | WAT | 4221 | −25.150 | 4.421 | 48.986 | 1.00 | 31.32 |
| ATOM | 21152 | OH2 | WAT | 4222 | 4.603 | −15.946 | 39.930 | 1.00 | 28.11 |
| ATOM | 21153 | OH2 | WAT | 4223 | −19.155 | 22.017 | 52.855 | 1.00 | 36.51 |
| ATOM | 21154 | OH2 | WAT | 4224 | −1.225 | 27.223 | 40.496 | 1.00 | 34.03 |
| ATOM | 21155 | OH2 | WAT | 4225 | 9.323 | −6.838 | 44.620 | 1.00 | 32.56 |
| ATOM | 21156 | OH2 | WAT | 4226 | −13.348 | −40.907 | 25.217 | 1.00 | 36.02 |
| ATOM | 21157 | OH2 | WAT | 4227 | −29.811 | −16.829 | 55.472 | 1.00 | 40.39 |
| ATOM | 21158 | OH2 | WAT | 4228 | 1.208 | 13.403 | −12.804 | 1.00 | 25.50 |
| ATOM | 21159 | OH2 | WAT | 4229 | 31.515 | 42.188 | 14.797 | 1.00 | 36.19 |
| ATOM | 21160 | OH2 | WAT | 4230 | −22.435 | −40.140 | 8.269 | 1.00 | 32.85 |
| ATOM | 21161 | OH2 | WAT | 4231 | 4.884 | −8.621 | 38.037 | 1.00 | 29.97 |
| ATOM | 21162 | OH2 | WAT | 4232 | 41.136 | −12.337 | 43.000 | 1.00 | 34.40 |
| ATOM | 21163 | OH2 | WAT | 4233 | 22.346 | 33.851 | 68.574 | 1.00 | 47.30 |
| ATOM | 21164 | OH2 | WAT | 4234 | 6.737 | 5.652 | 40.043 | 1.00 | 42.41 |
| ATOM | 21165 | OH2 | WAT | 4235 | 5.196 | −13.093 | 37.173 | 1.00 | 33.25 |
| ATOM | 21166 | OH2 | WAT | 4236 | 5.818 | −1.680 | 14.657 | 1.00 | 40.01 |
| ATOM | 21167 | OH2 | WAT | 4237 | −5.653 | −17.045 | 3.161 | 1.00 | 38.26 |
| ATOM | 21168 | OH2 | WAT | 4238 | −22.623 | 5.252 | 41.872 | 1.00 | 32.25 |
| ATOM | 21169 | OH2 | WAT | 4239 | −13.321 | −28.124 | 62.372 | 1.00 | 31.95 |
| ATOM | 21170 | OH2 | WAT | 4240 | −22.968 | −6.416 | 35.146 | 1.00 | 22.64 |
| ATOM | 21171 | OH2 | WAT | 4241 | −27.579 | −29.214 | 23.224 | 1.00 | 28.40 |
| ATOM | 21172 | OH2 | WAT | 4242 | −17.589 | −1.229 | 71.448 | 1.00 | 40.90 |
| ATOM | 21173 | OH2 | WAT | 4243 | −1.591 | −26.804 | 3.938 | 1.00 | 30.62 |
| ATOM | 21174 | OH2 | WAT | 4244 | 11.074 | −44.495 | 29.557 | 1.00 | 40.76 |
| ATOM | 21175 | OH2 | WAT | 4245 | −4.840 | 12.652 | 24.363 | 1.00 | 35.77 |
| ATOM | 21176 | OH2 | WAT | 4246 | 47.599 | −6.430 | 55.516 | 1.00 | 36.89 |
| ATOM | 21177 | OH2 | WAT | 4247 | −28.043 | 2.899 | −0.586 | 1.00 | 42.68 |
| ATOM | 21178 | OH2 | WAT | 4248 | 39.767 | −37.649 | 16.094 | 1.00 | 42.67 |
| ATOM | 21179 | OH2 | WAT | 4249 | 22.684 | 29.859 | 5.493 | 1.00 | 39.59 |
| ATOM | 21180 | OH2 | WAT | 4250 | −8.757 | 40.956 | 1.091 | 1.00 | 32.81 |
| ATOM | 21181 | OH2 | WAT | 4251 | 40.067 | −45.503 | 20.265 | 1.00 | 35.71 |
| ATOM | 21182 | OH2 | WAT | 4252 | 26.027 | 1.180 | 40.411 | 1.00 | 31.92 |
| ATOM | 21183 | OH2 | WAT | 4253 | 17.254 | −13.580 | 32.677 | 1.00 | 37.95 |
| ATOM | 21184 | OH2 | WAT | 4254 | 38.172 | 16.681 | 33.750 | 1.00 | 36.90 |
| ATOM | 21185 | OH2 | WAT | 4255 | −23.882 | 11.970 | 4.626 | 1.00 | 35.75 |
| ATOM | 21186 | OH2 | WAT | 4256 | 24.484 | 29.594 | 17.149 | 1.00 | 36.63 |
| ATOM | 21187 | OH2 | WAT | 4257 | 14.330 | 14.056 | −7.663 | 1.00 | 32.35 |
| ATOM | 21188 | OH2 | WAT | 4258 | 9.933 | −26.844 | 55.259 | 1.00 | 34.01 |
| ATOM | 21189 | OH2 | WAT | 4259 | 12.646 | −9.356 | −24.468 | 1.00 | 43.12 |
| ATOM | 21190 | OH2 | WAT | 4260 | 40.273 | −5.490 | 1.123 | 1.00 | 31.20 |
| ATOM | 21191 | OH2 | WAT | 4261 | 17.384 | −0.963 | 15.304 | 1.00 | 40.38 |
| ATOM | 21192 | OH2 | WAT | 4262 | 7.357 | 1.338 | 55.482 | 1.00 | 34.09 |
| ATOM | 21193 | OH2 | WAT | 4263 | −7.701 | 28.368 | 40.512 | 1.00 | 31.74 |
| ATOM | 21194 | OH2 | WAT | 4264 | 6.424 | 28.970 | 12.284 | 1.00 | 35.75 |
| ATOM | 21195 | OH2 | WAT | 4265 | 28.293 | −7.969 | 59.997 | 1.00 | 37.02 |
| ATOM | 21196 | OH2 | WAT | 4266 | 33.471 | −50.990 | 21.231 | 1.00 | 34.39 |
| ATOM | 21197 | OH2 | WAT | 4267 | 36.422 | 1.406 | 67.198 | 1.00 | 37.21 |
| ATOM | 21198 | OH2 | WAT | 4268 | 4.155 | 15.109 | 20.451 | 1.00 | 40.58 |
| ATOM | 21199 | OH2 | WAT | 4269 | −26.153 | −3.024 | −4.695 | 1.00 | 24.77 |
| ATOM | 21200 | OH2 | WAT | 4270 | 30.499 | −25.454 | 72.705 | 1.00 | 38.88 |
| ATOM | 21201 | OH2 | WAT | 4271 | 4.763 | 51.788 | 4.914 | 1.00 | 27.90 |
| ATOM | 21202 | OH2 | WAT | 4272 | −11.498 | 46.346 | 14.162 | 1.00 | 32.61 |
| ATOM | 21203 | OH2 | WAT | 4273 | 39.036 | −19.110 | 16.994 | 1.00 | 32.98 |
| ATOM | 21204 | OH2 | WAT | 4274 | −6.998 | 36.928 | 26.870 | 1.00 | 43.35 |
| ATOM | 21205 | OH2 | WAT | 4275 | 46.948 | 7.501 | 34.599 | 1.00 | 24.81 |
| ATOM | 21206 | OH2 | WAT | 4276 | 50.983 | −8.055 | −5.708 | 1.00 | 40.55 |
| ATOM | 21207 | OH2 | WAT | 4277 | 19.452 | −39.708 | 61.023 | 1.00 | 34.30 |
| ATOM | 21208 | OH2 | WAT | 4278 | 7.397 | −30.621 | 39.404 | 1.00 | 33.65 |
| ATOM | 21209 | OH2 | WAT | 4279 | 13.876 | −1.133 | 12.109 | 1.00 | 41.50 |
| ATOM | 21210 | OH2 | WAT | 4280 | −0.160 | 20.020 | −0.572 | 1.00 | 32.16 |
| ATOM | 21211 | OH2 | WAT | 4281 | 45.793 | −19.678 | 50.360 | 1.00 | 35.75 |
| ATOM | 21212 | OH2 | WAT | 4282 | 47.155 | 27.281 | 14.452 | 1.00 | 38.21 |
| ATOM | 21213 | OH2 | WAT | 4283 | 8.531 | −12.525 | 69.846 | 1.00 | 42.83 |
| ATOM | 21214 | OH2 | WAT | 4284 | 38.230 | 21.846 | 69.235 | 1.00 | 34.37 |
| ATOM | 21215 | OH2 | WAT | 4285 | 49.714 | −15.553 | 2.722 | 1.00 | 31.07 |
| ATOM | 21216 | OH2 | WAT | 4286 | 8.106 | 30.082 | 54.050 | 1.00 | 33.73 |
| ATOM | 21217 | OH2 | WAT | 4287 | 23.244 | 32.218 | 50.889 | 1.00 | 33.29 |
| ATOM | 21218 | OH2 | WAT | 4288 | 17.581 | 21.404 | 35.067 | 1.00 | 43.77 |
| ATOM | 21219 | OH2 | WAT | 4289 | 19.334 | 9.501 | 37.368 | 1.00 | 34.18 |
| ATOM | 21220 | OH2 | WAT | 4290 | −32.573 | −19.162 | 54.321 | 1.00 | 42.08 |
| ATOM | 21221 | OH2 | WAT | 4291 | 36.584 | −48.023 | 13.443 | 1.00 | 37.99 |
| ATOM | 21222 | OH2 | WAT | 4292 | 53.608 | 2.609 | 45.918 | 1.00 | 29.33 |
| ATOM | 21223 | OH2 | WAT | 4293 | −28.355 | −3.724 | 11.537 | 1.00 | 36.13 |
| ATOM | 21224 | OH2 | WAT | 4294 | 29.801 | 34.256 | 66.452 | 1.00 | 42.75 |
| ATOM | 21225 | OH2 | WAT | 4295 | 37.391 | 14.555 | 21.327 | 1.00 | 29.90 |
| ATOM | 21226 | OH2 | WAT | 4296 | −0.961 | 22.126 | 13.597 | 1.00 | 30.11 |
| ATOM | 21227 | OH2 | WAT | 4297 | 31.707 | 0.427 | −19.155 | 1.00 | 40.22 |
| ATOM | 21228 | OH2 | WAT | 4298 | 31.457 | −20.855 | −17.283 | 1.00 | 37.42 |
| ATOM | 21229 | OH2 | WAT | 4299 | 25.101 | −17.682 | −14.424 | 1.00 | 32.68 |
| ATOM | 21230 | OH2 | WAT | 4300 | 20.756 | 8.915 | −7.538 | 1.00 | 38.06 |
| ATOM | 21231 | OH2 | WAT | 4301 | 8.289 | −46.227 | 33.339 | 1.00 | 36.23 |
| ATOM | 21232 | OH2 | WAT | 4302 | −30.592 | −2.404 | 23.454 | 1.00 | 36.26 |
| ATOM | 21233 | OH2 | WAT | 4303 | −0.554 | 12.427 | −16.863 | 1.00 | 29.01 |
| ATOM | 21234 | OH2 | WAT | 4304 | 30.322 | −21.630 | −20.939 | 1.00 | 33.55 |
| ATOM | 21235 | OH2 | WAT | 4305 | −15.891 | 14.303 | 49.053 | 1.00 | 41.02 |
| ATOM | 21236 | OH2 | WAT | 4306 | −5.861 | −24.996 | 39.922 | 1.00 | 37.54 |
| ATOM | 21237 | OH2 | WAT | 4307 | −18.592 | 5.594 | 59.709 | 1.00 | 44.08 |
| ATOM | 21238 | OH2 | WAT | 4308 | −13.782 | −29.666 | 2.092 | 1.00 | 36.94 |
| ATOM | 21239 | OH2 | WAT | 4309 | −0.006 | 10.614 | 60.001 | 1.00 | 43.52 |
| ATOM | 21240 | OH2 | WAT | 4310 | 32.762 | −9.491 | −23.904 | 1.00 | 33.22 |
| ATOM | 21241 | OH2 | WAT | 4311 | 38.244 | −3.752 | 7.396 | 1.00 | 36.21 |
| ATOM | 21242 | OH2 | WAT | 4312 | 46.881 | −21.498 | −8.257 | 1.00 | 46.78 |
| ATOM | 21243 | OH2 | WAT | 4313 | 29.381 | −44.133 | 36.141 | 1.00 | 38.69 |
| ATOM | 21244 | OH2 | WAT | 4314 | 44.697 | −1.657 | 57.305 | 1.00 | 27.49 |
| ATOM | 21245 | OH2 | WAT | 4315 | 3.764 | −16.191 | 73.425 | 1.00 | 46.00 |
| ATOM | 21246 | OH2 | WAT | 4316 | 1.552 | 2.561 | 15.840 | 1.00 | 39.61 |
| ATOM | 21247 | OH2 | WAT | 4317 | 22.489 | 10.399 | 26.199 | 1.00 | 41.03 |
| ATOM | 21248 | OH2 | WAT | 4318 | −28.455 | −3.493 | 20.182 | 1.00 | 29.54 |
| ATOM | 21249 | OH2 | WAT | 4319 | 22.485 | −54.018 | 16.709 | 1.00 | 30.01 |
| ATOM | 21250 | OH2 | WAT | 4320 | 17.660 | −27.711 | 52.097 | 1.00 | 38.58 |
| ATOM | 21251 | OH2 | WAT | 4321 | 56.912 | 13.287 | 51.121 | 1.00 | 44.62 |
| ATOM | 21252 | OH2 | WAT | 4322 | −21.611 | −37.255 | 5.058 | 1.00 | 25.63 |
| ATOM | 21253 | OH2 | WAT | 4323 | 36.941 | −3.556 | 52.524 | 1.00 | 35.09 |
| ATOM | 21254 | OH2 | WAT | 4324 | 13.800 | 6.771 | −21.931 | 1.00 | 38.56 |
| ATOM | 21255 | OH2 | WAT | 4325 | −30.772 | −37.278 | 18.011 | 1.00 | 47.52 |
| ATOM | 21256 | OH2 | WAT | 4326 | −31.800 | −32.961 | 17.765 | 1.00 | 43.84 |
| ATOM | 21257 | OH2 | WAT | 4327 | 42.719 | 27.673 | 55.936 | 1.00 | 44.26 |
| ATOM | 21258 | OH2 | WAT | 4328 | 45.545 | 11.091 | 29.384 | 1.00 | 35.10 |
| ATOM | 21259 | OH2 | WAT | 4329 | 25.956 | −50.998 | 23.510 | 1.00 | 34.18 |
| ATOM | 21260 | OH2 | WAT | 4330 | 37.100 | −20.795 | 16.046 | 1.00 | 44.28 |
| ATOM | 21261 | OH2 | WAT | 4331 | 22.014 | 2.258 | −19.556 | 1.00 | 33.51 |
| ATOM | 21262 | OH2 | WAT | 4332 | −22.317 | 18.441 | 5.383 | 1.00 | 36.71 |
| ATOM | 21263 | OH2 | WAT | 4333 | −10.019 | −27.346 | 26.266 | 1.00 | 26.18 |
| ATOM | 21264 | OH2 | WAT | 4334 | −9.502 | −49.756 | 12.668 | 1.00 | 45.74 |
| ATOM | 21265 | OH2 | WAT | 4335 | −20.898 | −40.436 | 24.117 | 1.00 | 33.74 |
| ATOM | 21266 | OH2 | WAT | 4336 | −19.836 | 21.671 | −18.554 | 1.00 | 46.45 |
| ATOM | 21267 | OH2 | WAT | 4337 | 1.661 | −6.925 | 34.463 | 1.00 | 36.93 |
| ATOM | 21268 | OH2 | WAT | 4338 | 42.933 | 18.805 | 72.619 | 1.00 | 34.61 |
| ATOM | 21269 | OH2 | WAT | 4339 | −0.593 | −4.255 | 21.944 | 1.00 | 32.00 |
| ATOM | 21270 | OH2 | WAT | 4340 | 6.393 | 20.185 | 3.149 | 1.00 | 33.81 |
| ATOM | 21271 | OH2 | WAT | 4341 | 15.614 | 24.362 | 5.634 | 1.00 | 46.42 |
| ATOM | 21272 | OH2 | WAT | 4342 | 11.089 | 39.084 | 58.882 | 1.00 | 38.94 |
| ATOM | 21273 | OH2 | WAT | 4343 | 30.841 | −39.067 | 34.205 | 1.00 | 44.48 |
| ATOM | 21274 | OH2 | WAT | 4344 | 35.060 | −10.488 | 67.360 | 1.00 | 46.38 |
| ATOM | 21275 | OH2 | WAT | 4345 | 3.613 | −41.917 | 8.912 | 1.00 | 42.89 |
| ATOM | 21276 | OH2 | WAT | 4346 | −10.943 | 42.604 | 43.714 | 1.00 | 40.74 |
| ATOM | 21277 | OH2 | WAT | 4347 | 16.343 | 51.120 | 19.143 | 1.00 | 39.64 |
| ATOM | 21278 | OH2 | WAT | 4348 | 3.496 | 14.826 | 36.570 | 1.00 | 28.52 |
| ATOM | 21279 | OH2 | WAT | 4349 | 46.266 | 37.133 | 2.322 | 1.00 | 37.96 |
| ATOM | 21280 | OH2 | WAT | 4350 | −11.265 | 42.564 | 22.557 | 1.00 | 35.16 |
| ATOM | 21281 | OH2 | WAT | 4351 | −17.123 | 13.946 | 41.412 | 1.00 | 37.12 |
| ATOM | 21282 | OH2 | WAT | 4352 | −29.129 | −17.466 | 5.755 | 1.00 | 37.27 |
| ATOM | 21283 | OH2 | WAT | 4353 | 31.454 | −25.540 | 69.971 | 1.00 | 28.84 |
| ATOM | 21284 | OH2 | WAT | 4354 | 29.447 | −7.929 | −21.989 | 1.00 | 31.86 |
| ATOM | 21285 | OH2 | WAT | 4355 | −16.353 | 12.502 | 47.101 | 1.00 | 30.21 |
| ATOM | 21286 | OH2 | WAT | 4356 | 12.706 | −13.430 | 21.716 | 1.00 | 35.82 |
| ATOM | 21287 | OH2 | WAT | 4357 | 7.648 | −13.329 | −10.841 | 1.00 | 33.89 |
| ATOM | 21288 | OH2 | WAT | 4358 | 44.573 | 15.736 | 67.094 | 1.00 | 35.84 |
| ATOM | 21289 | OH2 | WAT | 4359 | −12.313 | 23.838 | 12.610 | 1.00 | 35.05 |
| ATOM | 21290 | OH2 | WAT | 4360 | 5.658 | 11.876 | −23.706 | 1.00 | 41.15 |
| ATOM | 21291 | OH2 | WAT | 4361 | −14.272 | −30.125 | 22.863 | 1.00 | 24.72 |
| ATOM | 21292 | OH2 | WAT | 4362 | 5.304 | −48.318 | 25.587 | 1.00 | 39.16 |
| ATOM | 21293 | OH2 | WAT | 4363 | 30.700 | −35.265 | 45.218 | 1.00 | 38.38 |
| ATOM | 21294 | OH2 | WAT | 4364 | −28.043 | −13.694 | 45.466 | 1.00 | 34.76 |
| ATOM | 21295 | OH2 | WAT | 4365 | 36.395 | −45.668 | 27.357 | 1.00 | 32.81 |
| ATOM | 21296 | OH2 | WAT | 4366 | 45.633 | −16.075 | 9.631 | 1.00 | 27.97 |
| ATOM | 21297 | OH2 | WAT | 4367 | 5.551 | 1.705 | 57.364 | 1.00 | 37.70 |
| ATOM | 21298 | OH2 | WAT | 4368 | 4.335 | 33.487 | 73.208 | 1.00 | 34.45 |
| ATOM | 21299 | OH2 | WAT | 4369 | −16.577 | −5.490 | −18.466 | 1.00 | 39.46 |
| ATOM | 21300 | OH2 | WAT | 4370 | 44.958 | −2.975 | 40.294 | 1.00 | 41.84 |
| ATOM | 21301 | OH2 | WAT | 4371 | −11.628 | 28.838 | 13.039 | 1.00 | 42.95 |
| ATOM | 21302 | OH2 | WAT | 4372 | 40.841 | 14.763 | 27.236 | 1.00 | 45.00 |
| ATOM | 21303 | OH2 | WAT | 4373 | 10.333 | 28.827 | 73.761 | 1.00 | 37.30 |
| ATOM | 21304 | OH2 | WAT | 4374 | 1.040 | 49.430 | 3.854 | 1.00 | 40.14 |
| ATOM | 21305 | OH2 | WAT | 4375 | 16.228 | 2.460 | −24.363 | 1.00 | 29.98 |
| ATOM | 21306 | OH2 | WAT | 4376 | −10.817 | −39.207 | 26.234 | 1.00 | 28.48 |
| ATOM | 21307 | OH2 | WAT | 4377 | −13.169 | −26.151 | 69.385 | 1.00 | 42.50 |
| ATOM | 21308 | OH2 | WAT | 4378 | 17.911 | −12.633 | 35.282 | 1.00 | 30.10 |
| ATOM | 21309 | OH2 | WAT | 4379 | 9.833 | −18.262 | −5.406 | 1.00 | 33.81 |
| ATOM | 21310 | OH2 | WAT | 4380 | −23.233 | −9.084 | 34.300 | 1.00 | 30.72 |
| ATOM | 21311 | OH2 | WAT | 4381 | 52.131 | 29.747 | 28.709 | 1.00 | 41.21 |
| ATOM | 21312 | OH2 | WAT | 4382 | 1.375 | 6.960 | 58.261 | 1.00 | 35.75 |
| ATOM | 21313 | OH2 | WAT | 4383 | −31.130 | −3.570 | 19.795 | 1.00 | 39.36 |
| ATOM | 21314 | OH2 | WAT | 4384 | 37.817 | 3.006 | 69.575 | 1.00 | 41.33 |
| ATOM | 21315 | OH2 | WAT | 4385 | −4.563 | −16.096 | 0.551 | 1.00 | 37.46 |
| ATOM | 21316 | OH2 | WAT | 4386 | 4.037 | −3.919 | 18.274 | 1.00 | 32.56 |
| ATOM | 21317 | OH2 | WAT | 4387 | −21.141 | −1.984 | 26.707 | 1.00 | 33.58 |
| ATOM | 21318 | OH2 | WAT | 4388 | −22.717 | 1.730 | −11.319 | 1.00 | 44.33 |
| ATOM | 21319 | OH2 | WAT | 4389 | 38.993 | −41.394 | 21.353 | 1.00 | 26.94 |
| ATOM | 21320 | OH2 | WAT | 4390 | 35.457 | −6.585 | 23.306 | 1.00 | 41.41 |
| ATOM | 21321 | OH2 | WAT | 4391 | −28.515 | −11.015 | 44.907 | 1.00 | 44.09 |
| ATOM | 21322 | OH2 | WAT | 4392 | 21.162 | 12.941 | 61.076 | 1.00 | 33.81 |
| ATOM | 21323 | OH2 | WAT | 4393 | −15.197 | 2.825 | 26.060 | 1.00 | 39.04 |
| ATOM | 21324 | OH2 | WAT | 4394 | −15.450 | −25.101 | 26.034 | 1.00 | 25.87 |
| ATOM | 21325 | OH2 | WAT | 4395 | 2.390 | 19.780 | −0.161 | 1.00 | 34.76 |
| ATOM | 21326 | OH2 | WAT | 4396 | 43.487 | −25.747 | 59.438 | 1.00 | 35.57 |
| ATOM | 21327 | OH2 | WAT | 4397 | 48.629 | 36.856 | 21.672 | 1.00 | 44.39 |
| ATOM | 21328 | OH2 | WAT | 4398 | 17.100 | 12.356 | 40.201 | 1.00 | 40.21 |
| ATOM | 21329 | OH2 | WAT | 4399 | 4.507 | −10.245 | −15.502 | 1.00 | 39.12 |
| ATOM | 21330 | OH2 | WAT | 4400 | −27.080 | 5.572 | 0.821 | 1.00 | 39.35 |
| ATOM | 21331 | OH2 | WAT | 4401 | −3.672 | 0.984 | 37.698 | 1.00 | 39.93 |
| ATOM | 21332 | OH2 | WAT | 4402 | −20.583 | 18.697 | 49.997 | 1.00 | 34.01 |
| ATOM | 21333 | OH2 | WAT | 4403 | 39.072 | −14.613 | 58.597 | 1.00 | 31.42 |
| ATOM | 21334 | OH2 | WAT | 4404 | 29.709 | 12.185 | 3.398 | 1.00 | 44.25 |
| ATOM | 21335 | OH2 | WAT | 4405 | 35.880 | −22.841 | 12.910 | 1.00 | 33.52 |
| ATOM | 21336 | OH2 | WAT | 4406 | −35.845 | −12.885 | 22.300 | 1.00 | 34.99 |
| ATOM | 21337 | OH2 | WAT | 4407 | 15.319 | 26.997 | 5.574 | 1.00 | 32.51 |
| ATOM | 21338 | OH2 | WAT | 4408 | 8.333 | 17.040 | 3.228 | 1.00 | 40.92 |
| ATOM | 21339 | OH2 | WAT | 4409 | −10.936 | 26.210 | 13.895 | 1.00 | 37.53 |
| ATOM | 21340 | OH2 | WAT | 4410 | 55.583 | 6.631 | 16.381 | 1.00 | 45.01 |
| ATOM | 21341 | OH2 | WAT | 4411 | 27.248 | −0.852 | 16.193 | 1.00 | 32.03 |
| ATOM | 21342 | OH2 | WAT | 4412 | 11.166 | −30.270 | 54.430 | 1.00 | 35.34 |
| ATOM | 21343 | OH2 | WAT | 4413 | 27.193 | 30.827 | 45.265 | 1.00 | 48.03 |
| ATOM | 21344 | OH2 | WAT | 4414 | 1.816 | −4.001 | 33.376 | 1.00 | 38.02 |
| ATOM | 21345 | OH2 | WAT | 4415 | −7.358 | 6.238 | 21.426 | 1.00 | 31.68 |
| ATOM | 21346 | OH2 | WAT | 4416 | 23.525 | 29.907 | 2.579 | 1.00 | 40.00 |
| ATOM | 21347 | OH2 | WAT | 4417 | 19.207 | 7.060 | −17.753 | 1.00 | 45.08 |
| ATOM | 21348 | OH2 | WAT | 4418 | 27.504 | 25.535 | 33.468 | 1.00 | 36.75 |
| ATOM | 21349 | OH2 | WAT | 4419 | 6.892 | −32.295 | 41.776 | 1.00 | 41.26 |
| ATOM | 21350 | OH2 | WAT | 4420 | 17.067 | −1.474 | 58.088 | 1.00 | 34.61 |
| ATOM | 21351 | OH2 | WAT | 4421 | 22.907 | 29.267 | 14.656 | 1.00 | 39.78 |
| ATOM | 21352 | OH2 | WAT | 4422 | 6.224 | −49.951 | 10.824 | 1.00 | 28.84 |
| ATOM | 21353 | OH2 | WAT | 4423 | −16.207 | 35.901 | 25.434 | 1.00 | 40.39 |
| ATOM | 21354 | OH2 | WAT | 4424 | 0.738 | 15.078 | 67.744 | 1.00 | 35.60 |
| ATOM | 21355 | OH2 | WAT | 4425 | −22.384 | 0.170 | 34.473 | 1.00 | 38.76 |
| ATOM | 21356 | OH2 | WAT | 4426 | 52.264 | 32.015 | 13.016 | 1.00 | 52.37 |
| ATOM | 21357 | OH2 | WAT | 4427 | 28.668 | −23.464 | −19.783 | 1.00 | 40.92 |
| ATOM | 21358 | OH2 | WAT | 4428 | 29.624 | 26.370 | 37.226 | 1.00 | 43.83 |
| ATOM | 21359 | OH2 | WAT | 4429 | 17.324 | −33.128 | 69.819 | 1.00 | 36.99 |
| ATOM | 21360 | OH2 | WAT | 4430 | 10.556 | 3.415 | 13.566 | 1.00 | 46.02 |
| ATOM | 21361 | OH2 | WAT | 4431 | 8.523 | 41.333 | 58.843 | 1.00 | 39.73 |
| ATOM | 21362 | OH2 | WAT | 4432 | 33.167 | 23.763 | 37.373 | 1.00 | 28.85 |
| ATOM | 21363 | OH2 | WAT | 4433 | 15.195 | −18.491 | −17.090 | 1.00 | 47.32 |
| ATOM | 21364 | OH2 | WAT | 4434 | −23.836 | 2.684 | 36.535 | 1.00 | 38.09 |
| ATOM | 21365 | OH2 | WAT | 4435 | 19.214 | 12.147 | −5.475 | 1.00 | 35.97 |
| ATOM | 21366 | OH2 | WAT | 4436 | 27.162 | −7.740 | 67.562 | 1.00 | 31.45 |
| ATOM | 21367 | OH2 | WAT | 4437 | −20.971 | −1.223 | 12.183 | 1.00 | 47.05 |
| ATOM | 21368 | OH2 | WAT | 4438 | 19.674 | 12.470 | 63.556 | 1.00 | 44.37 |
| ATOM | 21369 | OH2 | WAT | 4439 | −1.525 | 40.553 | 27.756 | 1.00 | 41.21 |
| ATOM | 21370 | OH2 | WAT | 4440 | −18.654 | 1.585 | 28.426 | 1.00 | 32.42 |
| ATOM | 21371 | OH2 | WAT | 4441 | 17.125 | 0.453 | 50.867 | 1.00 | 40.97 |
| ATOM | 21372 | OH2 | WAT | 4442 | 46.616 | −22.499 | −3.435 | 1.00 | 40.16 |
| ATOM | 21373 | OH2 | WAT | 4443 | 14.381 | 25.984 | 34.791 | 1.00 | 37.01 |
| ATOM | 21374 | OH2 | WAT | 4444 | −1.785 | −17.781 | 2.660 | 1.00 | 35.38 |
| ATOM | 21375 | OH2 | WAT | 4445 | −34.581 | −22.257 | 26.233 | 1.00 | 42.91 |
| ATOM | 21376 | OH2 | WAT | 4446 | 24.684 | 23.141 | 0.831 | 1.00 | 35.73 |
| ATOM | 21377 | OH2 | WAT | 4447 | 15.504 | 6.715 | 63.224 | 1.00 | 38.96 |
| ATOM | 21378 | OH2 | WAT | 4448 | 19.904 | 21.033 | 71.538 | 1.00 | 43.76 |
| ATOM | 21379 | OH2 | WAT | 4449 | −11.227 | 22.621 | −8.864 | 1.00 | 27.21 |
| ATOM | 21380 | OH2 | WAT | 4450 | 22.345 | 0.986 | 14.851 | 1.00 | 34.11 |
| ATOM | 21381 | OH2 | WAT | 4451 | −10.539 | −25.144 | 27.450 | 1.00 | 36.84 |
| ATOM | 21382 | OH2 | WAT | 4452 | 16.140 | 25.932 | 32.224 | 1.00 | 35.40 |
| ATOM | 21383 | OH2 | WAT | 4453 | 44.961 | 30.241 | 15.396 | 1.00 | 37.99 |
| ATOM | 21384 | OH2 | WAT | 4454 | 34.177 | −25.015 | 12.520 | 1.00 | 36.11 |
| ATOM | 21385 | OH2 | WAT | 4455 | −20.127 | −20.201 | 41.569 | 1.00 | 32.33 |
| ATOM | 21386 | OH2 | WAT | 4456 | 5.429 | −56.224 | 24.411 | 1.00 | 42.80 |
| ATOM | 21387 | OH2 | WAT | 4457 | 32.065 | 2.167 | 30.883 | 1.00 | 43.06 |
| ATOM | 21388 | OH2 | WAT | 4458 | 5.091 | 42.861 | 7.752 | 1.00 | 39.50 |
| ATOM | 21389 | OH2 | WAT | 4459 | −21.209 | −11.056 | 5.163 | 1.00 | 41.80 |
| ATOM | 21390 | OH2 | WAT | 4460 | 19.803 | 9.836 | 20.265 | 1.00 | 40.87 |
| ATOM | 21391 | OH2 | WAT | 4461 | 29.343 | 4.465 | 26.139 | 1.00 | 29.91 |
| ATOM | 21392 | OH2 | WAT | 4462 | 10.788 | 42.451 | 0.401 | 1.00 | 42.38 |
| ATOM | 21393 | OH2 | WAT | 4463 | −7.021 | −50.881 | 26.507 | 1.00 | 32.87 |
| ATOM | 21394 | OH2 | WAT | 4464 | −19.249 | 19.648 | 45.938 | 1.00 | 29.54 |
| ATOM | 21395 | OH2 | WAT | 4465 | 48.159 | 7.324 | 9.609 | 1.00 | 39.08 |
| ATOM | 21396 | OH2 | WAT | 4466 | 0.381 | −40.577 | 8.688 | 1.00 | 37.54 |
| ATOM | 21397 | OH2 | WAT | 4467 | −25.910 | −21.171 | 45.136 | 1.00 | 40.07 |
| ATOM | 21398 | OH2 | WAT | 4468 | −22.226 | −2.802 | 33.260 | 1.00 | 35.07 |
| ATOM | 21399 | OH2 | WAT | 4469 | −4.952 | 49.029 | 23.144 | 1.00 | 45.50 |
| ATOM | 21400 | OH2 | WAT | 4470 | 17.417 | 13.858 | −4.051 | 1.00 | 38.03 |
| ATOM | 21401 | OH2 | WAT | 4471 | 4.986 | 16.348 | −15.614 | 1.00 | 30.12 |
| ATOM | 21402 | OH2 | WAT | 4472 | 15.429 | −22.524 | 1.908 | 1.00 | 36.21 |
| ATOM | 21403 | OH2 | WAT | 4473 | 30.126 | −47.999 | 29.175 | 1.00 | 23.74 |
| ATOM | 21404 | OH2 | WAT | 4474 | −19.245 | 3.242 | 52.458 | 1.00 | 33.15 |
| ATOM | 21405 | OH2 | WAT | 4475 | 41.831 | 21.851 | 70.956 | 1.00 | 36.15 |
| ATOM | 21406 | OH2 | WAT | 4476 | 25.141 | −23.572 | −20.451 | 1.00 | 35.05 |
| ATOM | 21407 | OH2 | WAT | 4477 | −23.878 | 4.412 | −11.442 | 1.00 | 28.26 |
| ATOM | 21408 | OH2 | WAT | 4478 | 42.991 | −24.831 | 47.594 | 1.00 | 35.61 |
| ATOM | 21409 | OH2 | WAT | 4479 | −7.137 | 30.657 | 69.022 | 1.00 | 47.25 |
| ATOM | 21410 | OH2 | WAT | 4480 | 17.754 | 1.462 | 57.307 | 1.00 | 35.59 |
| ATOM | 21411 | OH2 | WAT | 4481 | −7.414 | 21.345 | −1.587 | 1.00 | 30.73 |
| ATOM | 21412 | OH2 | WAT | 4482 | 49.815 | 21.351 | 58.077 | 1.00 | 37.51 |
| ATOM | 21413 | OH2 | WAT | 4483 | −15.491 | 44.459 | 7.923 | 1.00 | 50.41 |
| ATOM | 21414 | OH2 | WAT | 4484 | 4.048 | −22.551 | 5.047 | 1.00 | 36.58 |
| ATOM | 21415 | OH2 | WAT | 4485 | 50.672 | −12.564 | −0.579 | 1.00 | 37.00 |
| ATOM | 21416 | OH2 | WAT | 4486 | 2.078 | −13.615 | 27.066 | 1.00 | 19.93 |
| ATOM | 21417 | OH2 | WAT | 4487 | −8.044 | 32.117 | 70.958 | 1.00 | 27.03 |
| ATOM | 21418 | OH2 | WAT | 4488 | 0.643 | −25.729 | 20.083 | 1.00 | 22.99 |
| ATOM | 21419 | OH2 | WAT | 4489 | 17.072 | 17.442 | 39.782 | 1.00 | 27.80 |
| ATOM | 21420 | OH2 | WAT | 4490 | 39.386 | −43.716 | 22.448 | 1.00 | 25.25 |
| ATOM | 21421 | OH2 | WAT | 4491 | 50.725 | −6.820 | 55.355 | 1.00 | 43.84 |
| ATOM | 21422 | OH2 | WAT | 4492 | −19.456 | 26.078 | 48.234 | 1.00 | 34.33 |
| ATOM | 21423 | OH2 | WAT | 4493 | 29.077 | −49.519 | 9.320 | 1.00 | 36.75 |
| ATOM | 21424 | OH2 | WAT | 4494 | 9.459 | −18.005 | 2.269 | 1.00 | 34.78 |
| ATOM | 21425 | OH2 | WAT | 4495 | −19.163 | 36.702 | 49.212 | 1.00 | 32.59 |
| ATOM | 21426 | OH2 | WAT | 4496 | 16.195 | 7.713 | −20.690 | 1.00 | 46.10 |
| ATOM | 21427 | OH2 | WAT | 4497 | 26.413 | −5.902 | 59.650 | 1.00 | 37.00 |
| ATOM | 21428 | OH2 | WAT | 4498 | −22.773 | 19.390 | 2.650 | 1.00 | 33.05 |
| ATOM | 21429 | OH2 | WAT | 4499 | 1.717 | −30.055 | 70.167 | 1.00 | 44.59 |
| ATOM | 21430 | OH2 | WAT | 4500 | −16.732 | 24.403 | −6.733 | 1.00 | 39.96 |
| ATOM | 21431 | OH2 | WAT | 4501 | 46.171 | −20.860 | 3.372 | 1.00 | 42.41 |
| ATOM | 21432 | OH2 | WAT | 4502 | 3.145 | 16.448 | 33.272 | 1.00 | 30.34 |
| ATOM | 21433 | OH2 | WAT | 4503 | 3.333 | −30.786 | 61.572 | 1.00 | 33.43 |
| ATOM | 21434 | OH2 | WAT | 4504 | 52.805 | 18.720 | 57.784 | 1.00 | 39.33 |
| ATOM | 21435 | OH2 | WAT | 4505 | 31.760 | −29.715 | 16.803 | 1.00 | 42.71 |
| ATOM | 21436 | OH2 | WAT | 4506 | 27.158 | −0.019 | −2.171 | 1.00 | 47.55 |
| ATOM | 21437 | OH2 | WAT | 4507 | 16.391 | −6.880 | 13.945 | 1.00 | 27.93 |
| ATOM | 21438 | OH2 | WAT | 4508 | −11.329 | 8.325 | 33.144 | 1.00 | 35.74 |
| ATOM | 21439 | OH2 | WAT | 4509 | −34.565 | −18.654 | 31.647 | 1.00 | 41.23 |
| ATOM | 21440 | OH2 | WAT | 4510 | 8.119 | 2.472 | 45.194 | 1.00 | 35.82 |
| ATOM | 21441 | OH2 | WAT | 4511 | 26.135 | −26.276 | 1.441 | 1.00 | 43.41 |
| ATOM | 21442 | OH2 | WAT | 4512 | 6.185 | 13.143 | 27.534 | 1.00 | 38.63 |
| ATOM | 21443 | OH2 | WAT | 4513 | 15.834 | −35.849 | 46.381 | 1.00 | 39.03 |
| ATOM | 21444 | OH2 | WAT | 4514 | 52.437 | 25.745 | 6.497 | 1.00 | 41.04 |
| ATOM | 21445 | OH2 | WAT | 4515 | 5.343 | 4.118 | 14.468 | 1.00 | 33.27 |
| ATOM | 21446 | OH2 | WAT | 4516 | 53.362 | 6.451 | 39.554 | 1.00 | 38.27 |
| ATOM | 21447 | OH2 | WAT | 4517 | 1.590 | −4.443 | −22.357 | 1.00 | 33.22 |
| ATOM | 21448 | OH2 | WAT | 4518 | 49.556 | −1.588 | 49.431 | 1.00 | 34.00 |
| ATOM | 21449 | OH2 | WAT | 4519 | −28.945 | 3.556 | 61.373 | 1.00 | 43.53 |
| ATOM | 21450 | OH2 | WAT | 4520 | −12.490 | 17.160 | 20.670 | 1.00 | 39.70 |
| ATOM | 21451 | OH2 | WAT | 4521 | 40.918 | −3.831 | 38.596 | 1.00 | 26.45 |
| ATOM | 21452 | OH2 | WAT | 4522 | 10.399 | −3.808 | 63.636 | 1.00 | 27.56 |
| ATOM | 21453 | OH2 | WAT | 4523 | −5.893 | −42.115 | 6.281 | 1.00 | 43.08 |
| ATOM | 21454 | OH2 | WAT | 4524 | −30.106 | −0.391 | 45.625 | 1.00 | 36.74 |
| ATOM | 21455 | OH2 | WAT | 4525 | 41.777 | 0.678 | −15.445 | 1.00 | 37.86 |
| ATOM | 21456 | OH2 | WAT | 4526 | 14.086 | 15.973 | 37.228 | 1.00 | 29.86 |
| ATOM | 21457 | OH2 | WAT | 4527 | −22.519 | 27.041 | 47.602 | 1.00 | 45.34 |
| ATOM | 21458 | OH2 | WAT | 4528 | −11.576 | 3.521 | −21.126 | 1.00 | 26.78 |
| ATOM | 21459 | OH2 | WAT | 4529 | −2.842 | 24.071 | 13.955 | 1.00 | 37.20 |
| ATOM | 21460 | OH2 | WAT | 4530 | 36.314 | −37.379 | 7.290 | 1.00 | 36.68 |
| ATOM | 21461 | OH2 | WAT | 4531 | 8.937 | 41.604 | 63.559 | 1.00 | 44.67 |
| ATOM | 21462 | OH2 | WAT | 4532 | 6.131 | −15.555 | 37.510 | 1.00 | 35.99 |
| ATOM | 21463 | OH2 | WAT | 4533 | 2.582 | −30.744 | 64.478 | 1.00 | 33.23 |
| ATOM | 21464 | OH2 | WAT | 4534 | 14.122 | 25.572 | 3.018 | 1.00 | 45.34 |
| ATOM | 21465 | OH2 | WAT | 4535 | 29.990 | 5.778 | −14.327 | 1.00 | 46.38 |
| ATOM | 21466 | OH2 | WAT | 4536 | 17.429 | −9.357 | 65.683 | 1.00 | 44.69 |
| ATOM | 21467 | OH2 | WAT | 4537 | 6.231 | −17.310 | 74.869 | 1.00 | 34.09 |
| ATOM | 21468 | OH2 | WAT | 4538 | 11.057 | 11.153 | 65.196 | 1.00 | 43.68 |
| ATOM | 21469 | OH2 | WAT | 4539 | −13.688 | 21.726 | 22.028 | 1.00 | 48.02 |
| ATOM | 21470 | OH2 | WAT | 4540 | −31.249 | −5.613 | 11.556 | 1.00 | 34.90 |
| ATOM | 21471 | OH2 | WAT | 4541 | −7.066 | 28.532 | 37.518 | 1.00 | 37.48 |
| ATOM | 21472 | OH2 | WAT | 4542 | 23.003 | 24.425 | 17.044 | 1.00 | 35.33 |
| ATOM | 21473 | OH2 | WAT | 4543 | −3.469 | 27.584 | 73.860 | 1.00 | 39.05 |
| ATOM | 21474 | OH2 | WAT | 4544 | 35.891 | 29.059 | 44.547 | 1.00 | 41.31 |
| ATOM | 21475 | OH2 | WAT | 4545 | 18.800 | −1.059 | 10.975 | 1.00 | 15.84 |
| ATOM | 21476 | OH2 | WAT | 4546 | −19.212 | −0.670 | 24.770 | 1.00 | 29.47 |
| ATOM | 21477 | OH2 | WAT | 4547 | −16.028 | −8.734 | −10.667 | 1.00 | 40.15 |
| ATOM | 21478 | OH2 | WAT | 4548 | 5.835 | −15.346 | 20.455 | 1.00 | 23.89 |
| ATOM | 21479 | OH2 | WAT | 4549 | 42.287 | −4.979 | 41.504 | 1.00 | 27.98 |
| ATOM | 21480 | OH2 | WAT | 4550 | 38.305 | 9.987 | 71.347 | 1.00 | 48.01 |
| ATOM | 21481 | OH2 | WAT | 4551 | −11.381 | 38.933 | 24.989 | 1.00 | 49.32 |
| ATOM | 21482 | OH2 | WAT | 4552 | −22.300 | 2.992 | 33.226 | 1.00 | 45.30 |
| ATOM | 21483 | OH2 | WAT | 4553 | 6.931 | −6.229 | 41.554 | 1.00 | 41.06 |
| ATOM | 21484 | OH2 | WAT | 4554 | 19.682 | −8.377 | 66.797 | 1.00 | 45.95 |
| ATOM | 21485 | OH2 | WAT | 4555 | 36.306 | −36.168 | 62.243 | 1.00 | 35.31 |
| ATOM | 21486 | OH2 | WAT | 4556 | 54.304 | 7.728 | 12.240 | 1.00 | 34.26 |
| ATOM | 21487 | OH2 | WAT | 4557 | −28.537 | 7.332 | 49.621 | 1.00 | 40.13 |
| ATOM | 21488 | OH2 | WAT | 4558 | 17.625 | 9.030 | 18.391 | 1.00 | 42.71 |
| ATOM | 21489 | OH2 | WAT | 4559 | −12.739 | 20.162 | 7.607 | 1.00 | 32.46 |
| ATOM | 21490 | OH2 | WAT | 4560 | −12.182 | 40.553 | 51.371 | 1.00 | 40.44 |
| ATOM | 21491 | OH2 | WAT | 4561 | 17.431 | −17.321 | 20.994 | 1.00 | 19.57 |
| ATOM | 21492 | OH2 | WAT | 4562 | 9.509 | 14.689 | 21.947 | 1.00 | 18.00 |
| ATOM | 21493 | OH2 | WAT | 4563 | 19.197 | 17.919 | 37.745 | 1.00 | 39.62 |
| ATOM | 21494 | OH2 | WAT | 4564 | 8.296 | 41.620 | 10.968 | 1.00 | 40.32 |
| ATOM | 21495 | OH2 | WAT | 4565 | 6.824 | 16.093 | −17.793 | 1.00 | 45.81 |
| ATOM | 21496 | OH2 | WAT | 4566 | −31.651 | −18.816 | 6.729 | 1.00 | 45.64 |
| ATOM | 21497 | OH2 | WAT | 4567 | −12.020 | 40.602 | 54.082 | 1.00 | 42.65 |
| ATOM | 21498 | OH2 | WAT | 4568 | −15.024 | 5.447 | −20.431 | 1.00 | 37.03 |
| ATOM | 21499 | OH2 | WAT | 4569 | 38.171 | −25.627 | −5.931 | 1.00 | 36.41 |
| ATOM | 21500 | OH2 | WAT | 4570 | 11.961 | 16.248 | 26.219 | 1.00 | 31.42 |
| ATOM | 21501 | OH2 | WAT | 4571 | 19.997 | 11.507 | 0.789 | 1.00 | 46.97 |
| ATOM | 21502 | OH2 | WAT | 4572 | −19.414 | −31.785 | 29.916 | 1.00 | 33.12 |
| ATOM | 21503 | OH2 | WAT | 4573 | 30.687 | 11.177 | 59.321 | 1.00 | 35.02 |
| ATOM | 21504 | OH2 | WAT | 4574 | −28.171 | 8.202 | −0.223 | 1.00 | 45.38 |
| ATOM | 21505 | OH2 | WAT | 4575 | −23.907 | −0.946 | 0.147 | 1.00 | 47.86 |
| ATOM | 21506 | OH2 | WAT | 4576 | 36.420 | −9.570 | −20.098 | 1.00 | 46.95 |
| ATOM | 21507 | OH2 | WAT | 4577 | −9.302 | 25.934 | −13.864 | 1.00 | 17.87 |
| ATOM | 21508 | OH2 | WAT | 4578 | 27.518 | −31.859 | 17.185 | 1.00 | 41.07 |
| ATOM | 21509 | OH2 | WAT | 4579 | 9.163 | 15.018 | 25.314 | 1.00 | 36.50 |
| ATOM | 21510 | OH2 | WAT | 4580 | 20.941 | 19.630 | 26.829 | 1.00 | 35.95 |
| ATOM | 21511 | OH2 | WAT | 4581 | 29.489 | −1.396 | −4.410 | 1.00 | 31.96 |
| ATOM | 21512 | OH2 | WAT | 4582 | 33.238 | 34.672 | 55.740 | 1.00 | 46.22 |
| ATOM | 21513 | OH2 | WAT | 4583 | −30.816 | −5.894 | 17.338 | 1.00 | 49.72 |
| ATOM | 21514 | OH2 | WAT | 4584 | 19.340 | −28.094 | 76.769 | 1.00 | 34.85 |
| ATOM | 21515 | OH2 | WAT | 4585 | −2.179 | 15.453 | 30.418 | 1.00 | 40.59 |
| ATOM | 21516 | OH2 | WAT | 4586 | 4.279 | −17.186 | 76.947 | 1.00 | 48.31 |
| ATOM | 21517 | OH2 | WAT | 4587 | −0.036 | −14.038 | 25.792 | 1.00 | 21.21 |
| ATOM | 21518 | OH2 | WAT | 4588 | 29.067 | −1.678 | 2.086 | 1.00 | 24.42 |
| ATOM | 21519 | OH2 | WAT | 4589 | −5.344 | 11.926 | 31.843 | 1.00 | 29.45 |
| ATOM | 21520 | OH2 | WAT | 4590 | −3.000 | −4.011 | −20.507 | 1.00 | 33.38 |
| ATOM | 21521 | OH2 | WAT | 4591 | 16.416 | −34.157 | 13.726 | 1.00 | 32.70 |
| ATOM | 21522 | OH2 | WAT | 4592 | 42.496 | 30.507 | 62.292 | 1.00 | 31.93 |
| ATOM | 21523 | OH2 | WAT | 4593 | 45.544 | −3.876 | 0.068 | 1.00 | 37.84 |
| ATOM | 21524 | OH2 | WAT | 4594 | 4.284 | 25.647 | 44.152 | 1.00 | 46.08 |
| ATOM | 21525 | OH2 | WAT | 4595 | 28.936 | 0.551 | −17.645 | 1.00 | 49.26 |
| ATOM | 21526 | OH2 | WAT | 4596 | 28.309 | −1.764 | −7.219 | 1.00 | 34.20 |
| ATOM | 21527 | OH2 | WAT | 4597 | −0.139 | −4.683 | −4.704 | 1.00 | 40.66 |
| ATOM | 21528 | OH2 | WAT | 4598 | 38.002 | −39.295 | 28.778 | 1.00 | 37.91 |
| ATOM | 21529 | OH2 | WAT | 4599 | 1.896 | −10.498 | 26.814 | 1.00 | 40.28 |
| ATOM | 21530 | OH2 | WAT | 4600 | 10.875 | −31.201 | 46.215 | 1.00 | 46.59 |
| ATOM | 21531 | OH2 | WAT | 4601 | −17.789 | 11.593 | 42.545 | 1.00 | 40.33 |
| ATOM | 21532 | OH2 | WAT | 4602 | 13.281 | −34.722 | 45.502 | 1.00 | 43.03 |
| ATOM | 21533 | OH2 | WAT | 4603 | 15.546 | 2.812 | 48.750 | 1.00 | 34.98 |
| ATOM | 21534 | OH2 | WAT | 4604 | −24.408 | −0.962 | 68.815 | 1.00 | 34.14 |
| ATOM | 21535 | OH2 | WAT | 4605 | 27.530 | 8.533 | −3.110 | 1.00 | 46.99 |
| ATOM | 21536 | OH2 | WAT | 4606 | −5.207 | −2.672 | −4.358 | 1.00 | 37.50 |
| ATOM | 21537 | OH2 | WAT | 4607 | −6.846 | −4.949 | 54.526 | 1.00 | 33.19 |
| ATOM | 21538 | OH2 | WAT | 4608 | −8.773 | −28.684 | 66.448 | 1.00 | 41.34 |
| ATOM | 21539 | OH2 | WAT | 4609 | 15.694 | −33.293 | 11.109 | 1.00 | 42.89 |
| ATOM | 21540 | OH2 | WAT | 4610 | 25.189 | 7.699 | −4.734 | 1.00 | 38.08 |
| ATOM | 21541 | OH2 | WAT | 4611 | −25.724 | −20.312 | 42.518 | 1.00 | 44.59 |
| ATOM | 21542 | OH2 | WAT | 4612 | 30.872 | −53.539 | 20.326 | 1.00 | 48.78 |
| ATOM | 21543 | OH2 | WAT | 4613 | 7.005 | −17.629 | 0.602 | 1.00 | 47.16 |
| ATOM | 21544 | OH2 | WAT | 4614 | −20.076 | 21.041 | 2.976 | 1.00 | 50.56 |
| ATOM | 21545 | OH2 | WAT | 4615 | 49.790 | 17.372 | −5.207 | 1.00 | 53.06 |
| ATOM | 21546 | OH2 | WAT | 4616 | 45.033 | 0.193 | −5.524 | 1.00 | 36.67 |
| ATOM | 21547 | OH2 | WAT | 4617 | 3.384 | 12.364 | 26.877 | 1.00 | 47.24 |
| ATOM | 21548 | OH2 | WAT | 4618 | −23.460 | −11.122 | 39.780 | 1.00 | 36.40 |
| ATOM | 21549 | OH2 | WAT | 4619 | 36.510 | −18.235 | −14.040 | 1.00 | 44.41 |
| ATOM | 21550 | OH2 | WAT | 4620 | 27.674 | 11.270 | 60.667 | 1.00 | 36.19 |
| ATOM | 21551 | OH2 | WAT | 4621 | −25.671 | −31.027 | 5.734 | 1.00 | 35.54 |
| ATOM | 21552 | OH2 | WAT | 4622 | −33.918 | −10.844 | 11.974 | 1.00 | 43.33 |
| END | | | | | | | | | |

TABLE 2
Crystallographic data quality, phasing, refinement and model quality

| | | | | P2₁ a = 86.1 |
|---|---|---|---|---|
| Space group & | | | | b = 157.2 |
| Cell parameters | P2₁ a = 87.8 b = 155.4 | | | c = 100.2 |
| (Å) | c = 209.9 β = 99.3° | | | β = 97.4° |
| Data quality | | | | |
| Data set | Edge | Peak | Remote | Native |
| Wavelength (Å) | 0.97939 | 0.97927 | 0.9393 | 0.979 |
| Limiting | 3.1 | 2.8 | 2.8 | 1.8 |
| resolution (Å) | | | | |
| Rₘₑₐₛ^{a} | 0.161 | 0.120 | 0.131 | |
| <I/σI> (high | 12.8 | 25.6 | 13.3 | 15.9 |
| resolution) | (2.6) | (6.0) | (3.3) | (2.1) |
| Completeness | 0.994 | 0.999 | 1.0 | 0.94 |
| No. unique | 100.734 | 136.609 | 136.664 | 229.086 |
| reflexions | (3.5) | (10.6) | (3.3) | (4.5) |
| (multiplicity) | | | | |
| Experimental | −9.9/2.9 | −8.6/5.4 | −1.3/3.2 | — |
| ƒ′/ƒ″ (electrons)^{b} | | | | |
| Refinement | | | | |
| (40-1.7 Å) | | | | |
| R_{cryst}^{c} | 0.229 | | | |
| | (highest | | | |
| | resolution: | | | |
| | 0.286) | | | |
| R_{free}^{d} | 0.263 | | | |
| | (0.318) | | | |
| No. reflexions: | 206.168/ | | | |
| working/test^{d} | 22.908 | | | |
| No. atoms | 19.820 | | | |
| (residues) | (2.640) | | | |
| No. waters | 1.610 | | | |
| Model quality | | | | |
| Ramachandran | | | | |
| plot: | | | | |
| % residues | 90.4 | | | |
| favourable | | | | |
| % unfavourable | None | | | |
| R.m.s. deviations: | | | | |
| Bond lengths | 0.006 | | | |
| Bond angles | 1.2 | | | |
| Dihedral angles | 22.1 | | | |

^{a}Rₘₑₐₛ = [ΣₕwΣᵢ|<Iₕ>−I_{h,t}|]/ΣₕΣᵢI_{h,t} where w = √[nₕ/(nₕ−1)] and <Iₕ> = [ΣₜⁿI_{h,t}]/nₕ. This is the multiplicity-weighted R_{symm} [Diederichs, 197 #155]
^{b}Estimates from CHOOCH (Evans, 1999)
^{c}R_{cryst} = Σ∥Fₒ|−|F_{c}∥/Σ|Fₒ|; Fₒ and F_{c} are observed and calculated structure factor amplitudes.
R_{free}: cross-validation R_{cryst}, i e calculated using randomly selected test data not used in refinement.

We claim:

1. A crystal of ketopantoate hydroxymethyltransferase (KPHMT) having a monoclinic space group P2₁, and unit cell dimensions of a=86.1±0.2 Å, b=157.2±0.2 Å, c=100.2±0.2 Å and β=97.4±0.2°.

2. A crystal of ketopantoate hydroxymethyltransferase having the three dimensional atomic coordinates of Table 1.

3. A method for crystallizing a selenomethionine KPHMT protein which comprises producing KPHMT by recombinant production in the presence of selenomethionine, recovering a selenomethionine KPHMT protein from the host and growing crystals therefrom.

4. A method of analysing a ketopantoate hydroxymethyl-transferase (KPHMT)-ligand complex comprising the step of employing (i) X-ray crystallographic diffraction data from the KPHMT-ligand complex and (ii) a three-dimensional structure of KPHMT to generate a difference Fourier electron density map of the complex, the three-dimensional structure being defined by atomic coordinate data according to Table 1.

5. A method for identifying an agent compound which modulates ketopantoate hydroxymethyltransferase (KPHMT) activity, comprising the steps of:
(a) employing three-dimensional atomic coordinate data according to Table 1 to characterise at least one KPHMT binding sites;
(b) providing the structure of a candidate agent compound;
(c) fitting the candidate agent compound to the binding sites; and
(d) selecting the candidate agent compound.

6. The method of claim 5 wherein:
a plurality of binding sites are characterised and a plurality of agent compounds are fitted to said sites; and
said agent compounds are linked to form a potential modulator compound.

7. The method of claim 5 wherein step (b) comprises selecting said candidate agent compound by computationally screening a database of compounds for interaction with said binding site.

8. The method of claim 5 which comprises the further steps of:
(e) obtaining or synthesising the candidate agent compound; and
(f) contacting the candidate agent compound with KPHMT to determine the ability of the candidate agent compound to interact with KPHMT.

9. The method of claim 5 which comprises the further steps of:
(e) obtaining or synthesising the candidate agent compound;
(f) forming a complex of KPHMT and the candidate agent compound; and
(g) analysing said complex by X-ray crystallography or NMR spectroscopy to determine the ability of the candidate agent compound to interact with KPHMT.

10. A compound which is identified as a modulator of KPHMT activity by the method of claim 5.

11. A method for determining the structure of a KPHMT homologue of the KPHMT defined by Table 1, wherein said method comprises:
(a) aligning a representation of an amino acid sequence of a KPHMT homologue of unknown structure with the amino acid sequence of KPHMT to match homologous regions of the amino acid sequences;
(b) modelling the structure of the matched homologous regions of the KPHMT of unknown structure on the structure as defined by Table 1 of the corresponding regions of the KPHMT of Table 1; and
(c) determining a conformation for the KPHMT of unknown structure which substantially preserves the structure of said matched homologous regions.

12. Computer readable media having atomic coordinate data according to Table 1 recorded thereon.

13. Computer readable media having structure factor data for KPHMT recorded thereon, the structure factor data being derivable from the atomic coordinate data of Table 1.
